(19) United States
(12) Patent Application Publication
(10) Pub. No.: US 2014/0256904 A1
(43) Pub. Date: Sep. 11, 2014

(54) BIOTECHNOLOGICAL SYNTHESIS PROCESS OF OMEGA-FUNCTIONALIZED CARBON ACIDS AND CARBON ACID ESTERS FROM SIMPLE CARBON SOURCES

(71) Applicants: Steffen Schaffer, Herten (DE); Nicole Decker, Recklinghausen (DE); Jasmin Gielen, Bochum (DE); Harald Haeger, Luedinghausen (DE); Thomas Haas, Muenster (DE); Markus Poetter, Shanghai (CN); Hans-Georg Hennemann, Marl (DE); Mirja Wessel, Bochum (DE); Michael Volland, Duelmen (DE)

(72) Inventors: Steffen Schaffer, Herten (DE); Nicole Decker, Recklinghausen (DE); Jasmin Gielen, Bochum (DE); Harald Haeger, Luedinghausen (DE); Thomas Haas, Muenster (DE); Markus Poetter, Shanghai (CN); Hans-Georg Hennemann, Marl (DE); Mirja Wessel, Bochum (DE); Michael Volland, Duelmen (DE)

(73) Assignee: Evonik Degussa GmbH, Essen (DE)

(21) Appl. No.: 14/238,591

(22) PCT Filed: Aug. 15, 2012
(86) PCT No.: PCT/EP12/65941

§ 371 Date: Feb. 12, 2014

(30) Foreign Application Priority Data
Aug. 15, 2011 (DE) 10 2011 110 946.7

(51) Int. Cl. C12P 13/00; C12P 13/02; C08G 69/08; C12P 7/64

(52) U.S. Cl. CPC C12P 13/005; C12P 7/6409; C12P 7/6436; C12P 13/02; C08G 69/08
USPC 528/310; 435/252.33; 435/252.34; 435/252.3; 435/252.32; 435/134; 435/129; 435/128

(57) ABSTRACT

The subject of the invention is a biotechnological process for the production of ω-functionalized carboxylic acids and of ω-functionalized carboxylate esters from simple carbon sources.

### FIELD OF INVENTION

The subject of the invention is a biotechnological process for the production of ω-functionalized carboxylic acids and ω-functionalized carboxylate esters from simple carbon sources.

### PRIOR ART

The biotechnological production of ω-functionalized carboxylic acids and the corresponding esters has previously been described exclusively in the form of biotransformations, wherein the corresponding carboxylic acid as substrate is contacted with an appropriate biological cell, which catalyses the necessary reactions enzymatically.

Such a process and cells which are suitable for the production of the ω-aminocarboxylic acids and esters thereof are for example described in WO2009077461.

EP2322598 also describes the production of ω-hydroxycarboxylic acids and esters thereof in specially equipped Candida tropicalis cells from fatty acids as the substrate used. A very similar procedure is described in WO2011008232, wherein Candida cells, wherein the β-oxidation is blocked, form corresponding ω-functionalized carboxylic acids and diacids starting from fatty acids by enzymatic oxidation.

The fatty acids and derivatives thereof required as substrates are mainly obtained nowadays exclusively from plant and animal oils or fats. This has a large number of disadvantages:

In particular, as a consequence of the BSE crisis, animal fats as raw materials still meet little client acceptance. Plant oils which contain short- and middle-length fatty acids are either difficult to obtain or are produced in tropical regions. Here the sustainability of production is in many cases called into question, since in some cases rainforest is cleared in order to provide the cultivation areas.

In addition, particular plant and animal oil or fat raw materials have specific, but defined fatty acid profiles. Hence coupled production takes place here, which can be price-determining for a certain fatty acid species. Last but not least, many of the plant oils are simultaneously also foodstuffs, so that under certain conditions a competition can arise between use as a substance and use as a foodstuff.

The purpose of the invention was to provide a biotechnological process for the production of ω-functionalized carboxylic acids and ω-functionalized carboxylate esters which is not reliant on fatty acids as educts.

### DESCRIPTION OF THE INVENTION

Surprisingly, it has been found that the cells described below containing genetic modifications are capable of solving the problem posed for the invention.

Hence a subject of the present invention are microorganisms which synthesize increased amounts of carboxylic acids or carboxylate esters and on the basis of further genetic features provide these with an ω-functionality.

A further subject of the invention is the use of the aforesaid microorganisms for the production of ω-functionalized carboxylic acids and ω-functionalized carboxylate esters and a process for the production of ω-functionalized carboxylic acids and ω-functionalized carboxylate esters with use of the microorganisms.

An advantage of the present invention is that the product inhibition in the production process can be markedly reduced.

A further advantage of the present invention is that the process enables the production of ω-functionalized carboxylic acids and ω-functionalized carboxylate esters from unrelated carbon sources with high space-time yield, high carbon yield and high concentration in the culture supernatant. As a result of the latter in particular, an efficient workup is facilitated.

The invention comprises methods for the generation of recombinant microbial cells which are capable of producing ω-functionalized carboxylic acids and ω-functionalized carboxylate esters from unrelated carbon sources.

The present invention thus comprises a microorganism, which has a first genetic modification, so that compared to its wild type it is capable of forming more carboxylic acid or carboxylate ester from at least one simple carbon source, characterized in that the microorganism has a second genetic modification, which comprises that the microorganism has, in comparison to its wild type, increased activity

of at least one enzyme E₁ which catalyses the conversion of carboxylic acids or carboxylate esters to the corresponding ω-hydroxycarboxylic acids or ω-hydroxycarboxylate esters.

In connection with the present invention, the term “a first genetic modification” is understood to mean at least one genetic engineering alteration of the microorganism, whereby the expression of one or more genes has been modified, i.e. increased or reduced, compared to the wild type strain.

In connection with the present invention, the term “simple carbon source” is understood to mean carbon sources wherein in the carbon skeleton at least one C—C bond has been broken and/or at least one carbon atom of the simple carbon source must form at least one new bond with at least one carbon atom of another molecule, in order to arrive at the carbon skeleton of the “more carboxylic acid or carboxylate ester”.

All stated percentages (%) are, unless otherwise stated, mass percent.

As the carbon source, carbohydrates such as for example glucose, saccharose, arabinose, xylose, lactose, fructose, maltose, molasses, starch, cellulose and hemicellulose, but also glycerin or very simple organic molecules such as CO₂, CO or synthesis gas, can be used.

Preferred carboxylic acids or carboxylate esters of the present invention are those which have more than one, in particular 3 to 36, preferably 6 to 24, in particular 10 to 14 carbon atoms in the carboxylic acid chain. This can be linear, branched, saturated or unsaturated and optionally substituted with other groups.

The carboxylate esters are preferably those wherein the alcohol component is derived from methanol, ethanol or other primary alcohols with 3-18 carbon atoms, in particular methanol and ethanol.

Particularly preferably, the carboxylic acids are fatty acids selected from the group formic acid, acetic acid, propionic acid, butyric acid, valeric acid, caproic acid, oenanthic acid, caprylic acid, pelargonic acid, capric acid, lauric acid, myristic acid, pentadecanoic acid, palmitic acid, margaric acid, stearic acid, nonadecanoic acid, arachic acid, behenic acid, lignoceric acid, cerotic acid, Montan acid, melissic acid, undecylenic acid, myristoleic acid, petroselic acid, oleic acid, elaidic acid, vaccenic acid, gadoleic acid, icosenic acid, cetoleic acid, erucic acid, nervoic acid, linolic acid, α-linolenic acid, γ-linolenic acid, calendula acid, punicic acid, α-elaeostearic acid, β-elaeostearic acid, arachidonic acid, timnodonic acid, clupanodonic acid, cervonic acid, vernolic acid, ricinoleic acid

and esters thereof, wherein the fatty acid esters are preferably those wherein the alcohol component is derived from methanol, ethanol or other primary alcohols with 3-18 carbon atoms, in particular methanol and ethanol.

It is preferable according to the invention that microorganisms are used on the basis of good genetic accessibility; selected from the group of the bacteria, particularly from the group containing, preferably consisting of, Abiotrophia, Acalyochloris, Accumulibacter, Acetivibrio, Acetobacter, Acetohalobium, Acetonema, Achromobacter, Acidaminococcus, Acidimicrobium, Acidiphilium, Acidithiobacillus, Acidobacterium, Acidothermus, Acidovorax, Acinetobacter, Actinobacillus, Actinomyces, Actinosynnema, Aerococcus, Aeromicrobium, Aeromonas, Afipia, Aggregatibacter, Agrobacterium, Ahrensia, Akkermansia, Alcanivorax, Alicycliphilus, Alicyclobacillus, Aliivibrio, Alkalilimnicola, Alkaliphilus, Allochromatium, Alteromonadales, Alteromonas, Aminobacterium, Aminomonas, Ammonifex, Amycolatopsis, Amycolicicoccus, Anabaena, Anaerobaculum, Anaerococcus, Anaerofustis, Anaerolinea, Anaeromyxobacter, Anaerostipes, Anaerotruncus, Anaplasma, Anoxybacillus, Aquifex, Arcanobacterium, Arcobacter, Aromatoleum, Arthrobacter, Arthrospira, Asticcacaulis, Atopobium, Aurantimonas, Azoarcus, Azorhizobium, Azospirillum, Azotobacter, Bacillus, Bartonella, Basfia, Baumannia, Bdellovibrio, Beggiatoa, Beijerinckia, Bermanella, Beutenbergia, Bifidobacterium, Bilophila, Blastopirellula, Blautia, Blochmannia, Bordetella, Borrelia, Brachybacterium, Brachyspira, Bradyrhizobium, Brevibacillus, Brevibacterium, Brevundimonas, Brucella, Buchnera, Bulleidia, Burkholderia, Butyrivibrio, Caldalkalibacillus, Caldanaerobacter, Caldicellulosiruptor, Calditerrivibrio, Caminibacter, Campylobacter, Carboxydibrachium, Carboxydothermus, Cardiobacterium, Carnobacterium, Carsonella, Catenibacterium, Catenulispora, Catonella, Caulobacter, Cellulomonas, Cellvibrio, Centipeda, Chelativorans, Chloroflexus, Chromobacterium, Chromohalobacter, Chthoniobacter, Citreicella, Citrobacter, Citromicrobium, Clavibacter, Cloacamonas, Clostridium, Collinsella, Colwellia, Comamonas, Conexibacter, Congregibacter, Coprobacillus, Coprococcus, Coprothermobacter, Coraliomargarita, Coriobacterium, corrodens, Corynebacterium, Coxiella, Crocosphaera, Cronobacter, Ctyptobacterium, Cupriavidus, Cyanobium, Cyanothece, Cylindrospermopsis, Dechloromonas, Deferribacter, Dehalococcoides, Dehalogenimonas, Deinococcus, Delftia, Denitrovibrio, Dermacoccus, Desmospora, Desulfarculus, Desulphateibacillum, Desulfitobacterium, Desulfobacca, Desulfobacterium, Desulfobulbus, Desulfococcus, Desulfohalobium, Desulfomicrobium, Desulfonatronospira, Desulforudis, Desulfotalea, Desulfotomaculum, Desulfovibrio, Desulfurispirillum, Desulfurobacterium, Desulfuromonas, Dethiobacter, Dethiosulfovibrio, Dialister, Dichelobacter, Dickeya, Dictyoglomus, Dietzia, Dinoroseobacter, Dorea, Edwardsiella, Ehrlichia, Eikenella, Elusimicrobium, Endoriftia, Enhydrobacter, Enterobacter, Enterococcus, Epulopiscium, Erwinia, Erysipelothrix, Etythrobacter, Escherichia, Ethanoligenens, Eubacterium, Eubacterium, Exiguobacterium, Faecalibacterium, Ferrimonas, Fervidobacterium, Fibrobacter, Finegoldia, Flexistipes, Francisella, Frankia, Fructobacillus, Fulvimarina, Fusobacterium, Gallibacterium, Gallionella, Gardnerella, Gemella, Gemmata, Gemmatimonas, Geobacillus, Geobacter, Geodermatophilus, Glaciecola, Gloeobacter, Glossina, Gluconacetobacter, Gordonia, Granulibacter, Granulicatella, Grimontia, Haemophilus, Hahella, Halanaerobiumns, Haliangium, Halomonas, Halorhodospira, Halothermothrix, Halothiobacillus, Hamiltonella, Helicobacter, Heliobacterium, Herbaspirillum, Herminiimonas, Herpetosiphon, Hippea, Hirschia, Histophilus, Hodgkinia, Hoeflea, Holdemania, Hydrogenivirga, Hydrogenobaculum, Hylemonella, Hyphomicrobium, Hyphomonas, Idiomarina, Ilyobacter, Intrasporangium, Isoptericola, Isosphaera, Janibacter, Janthinobacterium, Jonesia, Jonquetella, Kangiella, Ketogulonicigenium, Kineococcus, Kingella, Klebsiella, Kocuria, Koribacter, Kosmotoga, Kribbella, Ktedonobacter, Kytococcus, Labrenzia, Lactobacillus, Lactococcus, Laribacter, Lautropia, Lawsonia, Legionella, Leifsonia, Lentisphaera, Leptolyngbya, Leptospira, Leptothrix, Leptotrichia, Leuconostoc, Liberibacter, Limnobacter, Listeria, Loktanella, Lutiella, Lyngbya, Lysinibacillus, Macrococcus, Magnetococcus, Magnetospirillum, Mahella, Mannheimia, Maricaulis, Marinithermus, Marinobacter, Marinomonas, Mariprofundus, Maritimibacter, Marvinbryantia, Megasphaera, Meiothermus, Melissococcus, Mesorhizobium, Methylacidiphilum, Methylibium, Methylobacillus, Methylobacter, Methylobacterium, Methylococcus, Methylocystis, Methylomicrobium, Methylophaga, Methylophilales, Methylosinus, Methyloversatilis, Methylovorus, Microbacterium, Micrococcus, Microcoleus, Microcystis, Microlunatus, Micromonospora, Mitsuokella, Mobiluncus, MooreIla, Moraxella, Moritella, Mycobacterium, Myxococcus, Nakamurella, Natranaerobius, Neisseria, Neorickettsia, Neptuniibacter, Nitratifractor, Nitratiruptor, Nitrobacter, Nitrococcus, Nitrosomonas, Nitrosospira, Nitrospira, Nocardia, Nocardioides, Nocardiopsis, Nodularia, Nostoc, Novosphingobium, Oceanibulbus, Oceanicaulis, Oceanicola, Oceanithermus, Oceanobacillus, Ochrobactrum, Octadecabacter, Odyssella, Oligotropha, Olsenella, Opitutus, Oribacterium, Orientia, Ornithinibacillus, Oscillatoria, Oscillochloris, Oxalobacter, Paenibacillus, Pantoea, Paracoccus, Parascardovia, Parasutterella, Parvibaculum, Parvimonas, Parvularcula, Pasteurella, Pasteuria, Pectobacterium, Pediococcus, Pedosphaera, Pelagibaca, Pelagibacter, Pelobacter, Pelotomaculum, Peptoniphilus, Peptostreptococcus, Persephonella, Petrotoga, Phaeobacter, Phascolarctobacterium, Phenylobacterium, Photobacterium, Pirellula, Planctomyces, Planococcus, Plesiocystis, Polaromonas, Polaromonas, Polymorphum, Polynucleobacter, Poribacteria, Prochlorococcus, Propionibacterium, Proteus, Providencia, Pseudoalteromonas, Pseudo flavonifractor, Pseudomonas, Pseudonocardia, Pseudoramibacter, Pseudovibrio, Pseudoxanthomonas, Psychrobacter, Psychromonas, Puniceispirillum, Pusiffimonas, Pyramidobacter, Rahnella, Ralstonia, Raphidiopsis, Regiella, Reinekea, Renibacterium, Rhizobium, Rhodobacter, Rhodococcus, Rhodoferax, Rhodomicrobium, Rhodopirellula, Rhodopseudomonas, Rhodospirillum, Rickettsia, Rickettsiella, Riesia, Roseburia, Roseibium, Roseiflexus, Roseobacter, Roseomonas, Roseovarius, Rothia, Rubrivivax, Rubrobacter, Ruegeria, Ruminococcus, Ruthia, Saccharomonospora, Saccharophagus, Saccharopolyspora, Sagittula, Salinispora, Salmonella, Sanguibacte, Scardovia, Sebaldella, Segniliparus, Selenomonas, Serratia, Shewanella, Shigella, Shuttleworthia, Sideroxydans, Silicibacter, Simonsiella, Sinorhizobium, Slackia, Sodalis, Solibacter, Solobacterium, Sorangium, Sphaerobacter, Sphingobium, Sphingomonas, Sphingopyxis, Spirochaeta, Sporosarcina, Stackebrandtia, Staphylococcus, Starkeya, Stenotrophomonas, Stigmatella, Streptobacillus, Streptococcus, Streptomyces, Streptosporangium, Subdoligranulum, subvibrioides, Succinatimonas, Sulfitobacter, Sulfobacillus, Sulfuricurvum, Sulfurihydrogenibium, Sulfurimonas, Sulfurospirillum, Sulfurovum, Sutterella, Symbiobacterium, Synechocystis, Syntrophobacter, Syntrophobotulus, Syntrophomonas, Syntrophothermus, Syntrophus, taiwanensis, Taylorella, Teredinibacter, Terriglobus, Thalassiobium, Thauera, Thermaerobacter, Thermanaerovibrio, Thermincola, Thermoanaerobacter, Thermoanaerobacterium, Thermobaculum, Thermobifida, Thermobispora, Thermocrinis, Thermodesulphateator, Thermodesulfobacterium, Thermodesulfobium, Thermodesulfovibrio, Thermomicrobium, Thermomonospora, Thermosediminibacter, Thermosinus, Thermosipho, Thermosynechococcus, Thermotoga, Thermovibrio, Thermus, Thioalkalimicrobium, Thioalkalivibrio, Thiobacillus, Thiomicrospira, Thiomonas, Tolumonas, Treponema, tribocorum, Trichodesmium, Tropheryma, Truepera, Tsukamurella, Turicibacter, Variovorax, Veillonella, Verminephrobacter, Verrucomicrobium, Verrucosispora, Vesicomyosocius, Vibrio, Vibrionales, Victivallis, Weissella, Wigglesworthia, Wolbachia, Wolinella, Xanthobacter, Xanthomonas, Xenorhabdus, Xylanimonas, Xylella, Yersinia, Zinderia and Zymomonas, in particular E. coli, Pseudomonas sp., Pseudomonas fluorescens, Pseudomonas putida, Pseudomonas stutzeri, Acinetobacter sp., Burkholderia sp., Burkholderia thailandensis, Cyanobakterien, Klebsiella sp., Klebsiella oxytoca, Salmonella sp., Rhizobium sp. and Rhizobium meliloti, Bacillus sp., Bacillus subtilis, Clostridium sp., Corynebacterium sp., Corynebacterium glutamicum, Brevibacterium sp., Chlorella sp. and Nostoc sp., with E. coli being particularly preferable.

In comparison to its wild type, a microorganism according to the invention displays increased activity of at least one enzyme E₁.

The term “increased activity of an enzyme”, as it is used above and in the following explanations in connection with the present invention is preferably to be understood as increased intracellular activity.

The explanations now following concerning the increasing of the enzyme activity in cells apply both for the increasing of the activity of the enzyme E₁ and also for all enzymes mentioned below whose activity can optionally be increased, and also for increased formation of alkL gene product.

Essentially, an increase in the enzymatic activity can be achieved by increasing the copy number of the gene sequence or of the gene sequences which code for the enzyme, using a strong promoter, altering the codon utilization of the gene, increasing the half-life of the mRNA or of the enzyme in various ways, modifying the regulation of the expression of the gene or utilizing a gene or allele which codes for a corresponding enzyme with increased activity and optionally combining these measures. Microorganisms genetically modified according to the invention are for example created by transformation, transduction, conjugation or a combination of these methods with a vector which contains the desired gene, an allele of this gene or parts thereof and contains a promoter enabling the expression of the gene. The heterologous expression is in particular achieved by integration of the gene or the allele into the chromosome of the cell or an extrachromosomally replicating vector.

An overview of the possibilities for increasing the enzyme activity in cells, with pyruvate carboxylase as the example, is given DE-A-100 31 999, which is herewith incorporated as a reference and the disclosure content whereof forms one part of the disclosure of the present invention regarding the possibilities for increasing enzyme activity in cells.

The expression of the enzymes and genes mentioned above and all mentioned below is determinable by means of 1- and 2-dimensional protein gel separation followed by optical identification of the protein concentration in the gel with appropriate evaluation software.

If the increasing of an enzyme activity is based exclusively on increasing the expression of the corresponding gene, then the quantification of the increasing of the enzyme activity can be simply determined by a comparison of the 1- or 2-dimensional protein separations between wild type and genetically modified cell. A common method for the preparation of the protein gels with bacteria and for identification of the proteins is the procedure described by Hermann et al. (Electrophoresis, 22: 1712-23 (2001). The protein concentration can also be analysed by Western blot hybridization with an antibody specific for the protein to be determined (Sambrook et al., Molecular Cloning: a laboratory manual, 2nd Ed. Cold Spring Harbor Laboratory Press, Cold Spring Harbor, N.Y. USA, 1989) followed by optical evaluation with appropriate software for concentration determination (Lohaus and Meyer (1989) Biospektrum, 5: 32-39; Lottspeich (1999), Angewandte Chemie 111: 2630-2647).

This method is also always an option when possible products of the reaction to be catalysed by the enzyme activity to be determined may be rapidly metabolized in the microorganism or else the activity in the wild type is itself too low for it to be possible adequately to determine the enzyme activity to be determined on the basis of the production formation.

With regard to the enzyme E₁, the conversion of lauric acid and/or methyl laurate to ω-hydroxylauric acid and/or methyl ω-hydroxylaurate is understood in particular as a measure of the enzyme activity.

### Specified Enzymes E₁

It is preferable according to the invention if in the microorganism the enzyme E₁ is selected from the group:

E₁ₐ P450 alkane hydroxylases, which preferentially catalyse the following reactions: reduced haem+alkanoic acid (ester)=oxidized haem+ω-hydroxyalkanoic acid (ester)+H₂O,
2 reduced haem+alkanoic acid (ester)=2 oxidized haem+ω-oxoalkanoic acid (ester)+2H₂O or
3 reduced haem+alkanoic acid (ester)=alkane monooxygenase+3 oxidized haem+ω-carboxyalkanoic acid (ester)+3H₂O and preferably are
a component of a reaction system consisting of the two enzyme components “cytochrome P450 alkane hydroxylase and NADPH cytochrome P450 oxidoreductase of EC 1.6.2.4” or
a component of a reaction system consisting of the three enzyme components “cytochrome P450 alkane hydroxylase of the CYP_{—}153 type, ferredoxin NAD(P)⁺ reductases of EC 1.18.1.2 or EC 1.18.1.3 and ferredoxin”, and
E_{1b} AlkB alkane hydroxylases of EC 1.14.15.3, which preferentially catalyse the following reactions: reduced rubredoxin+alkanoic acid (ester)=oxidized rubredoxin+ω-hydroxyalkanoic acid (ester)+H₂O,
2 reduced rubredoxins+alkanoic acid (ester)=2 oxidized rubredoxins+ω-oxoalkanoic acid (ester)+2H₂O or
3 reduced rubredoxins+alkanoic acid (ester)=alkane monooxygenase+3 oxidized rubredoxins+ω-carboxyalkanoic acid (ester)+3H₂O and preferably are
a component of a reaction system consisting of the three enzyme components “AlkB alkane hydroxylase of EC 1.14.15.3, AlkT rubredoxin NAD(P)⁺ reductase of EC 1.18.1.1 or of EC 1.18.1.4 and rubredoxin AlkG”.

The access numbers stated in connection with the present invention correspond to the NCBI ProteinBank database entries with the date 26.07.2011; as a rule, the version number of the entry is identified here by “numerals” such as for example “0.1”.

P450 alkane hydroxylases preferred in this connection are selected from the list

AAO73954.1, AAO73953.1, XP_{—}002546279.1, AAA34353.2, P30607.1, XP_{—}002421627.1, XP_{—}718670.1, CAA39366.1, XP_{—}001527524.1, AAO73955.1, AAO73956.1, XP_{—}002546278.1, EEQ43157.1, XP_{—}718669.1, AAA34354.1, P10615.3, XP_{—}002421628.1, 226487, P16141.3, CAA39367.1, Q9Y757.2, XP_{—}001485567.1, AAO73958.1, XP_{—}001383506.2, XP_{—}460111.2, AAO73959.1, Q12586.1, XP_{—}460112.2, AAO73960.1, Q12589.1, AAO73961.1, XP_{—}460110.2, EEQ43763.1, XP_{—}710174.1, EDK41572.2, XP_{—}001482650.1, CAA75058.1, XP_{—}002548818.1, Q12588.1, XP_{—}002422222.1, XP_{—}001383636.2, XP_{—}001525381.1, XP_{—}002548823.1, P30610.1, AAO73952.1, XP_{—}002548428.1, CAA36197.1, XP_{—}002421126.1, AAA34320.1, P16496.3, P30608.1, P24458.1, XP_{—}717999.1, XP_{—}001383817.1, Q9Y758.1, XP_{—}001482092.1, XP_{—}001383710.2, P30609.1, AAB24479.1, XP_{—}457792.1, XP_{—}001524144.1, XP_{—}457727.2, XP_{—}001525578.1, XP_{—}002616743.1, XP_{—}002614836.1, XP_{—}001525577.1, AAO73957.1, Q12585.1, XP_{—}001386440.2, XP_{—}002616857.1, XP_{—}001483276.1, XP_{—}500402.1, EDK39907.2, XP_{—}500560.1, XP_{—}001211376.1, XP_{—}002560027.1, XP_{—}504857.1, XP_{—}500855.1, XP_{—}504406.1, BAA31433.1, XP_{—}500856.1, XP_{—}501148.1, XP_{—}746567.1, XP_{—}001262425.1, XP_{—}001274843.1, XP_{—}002840588.1, XP_{—}002377641.1, XP_{—}001825995.1, XP_{—}001400739.1, XP_{—}718066.1, CAA35593.1, XP_{—}664735.1, XP_{—}002150795.1, XP_{—}500097.1, XP_{—}002483325.1, XP_{—}504311.1, XP_{—}500273.1, XP_{—}002548817.1, EDP54484.1, XP_{—}755288.1, XP_{—}001260447.1, EFY97851.1, ACD75398.1, ADK36660.1, XP_{—}001213081.1, XP_{—}002377989.1, XP_{—}001826299.1, XP_{—}001554811.1, XP_{—}501667.1, XP_{—}002148942.1, ADK36662.1, XP_{—}002565827.1, P30611.1, XP_{—}001267871.1, XP_{—}002372373.1, EFY84686.1, P43083.1, XP_{—}001263094.1, XP_{—}002148355.1, XP_{—}002568429.1, XP_{—}001817314.1, Q12587.1, XP_{—}001396435.1, XP_{—}001938589.1, XP_{—}001388497.2, XP_{—}663661.1, XP_{—}003295335.1, XP_{—}002152088.1, XP_{—}001212071.1, Q12573.1, XP_{—}002379858.1, XP_{—}001821592.1, XP_{—}002844341.1, XP_{—}001394678.1, ACD75400.1, BAK03594.1, XP_{—}003170343.1, XP_{—}001265480.1, XP_{—}002550661.1, EDP55514.1, XP_{—}001528842.1, XP_{—}749919.1, XP_{—}001593058.1, P30612.1, EGC48494.1, EEH04429.1, XP_{—}001585586.1, XP_{—}003236182.1, XP_{—}001400199.1, EEQ46951.1, XP_{—}721410.1, EGP87864.1, XP_{—}002380808.1, XP_{—}001792771.1, XP_{—}001208515.1, XP_{—}001216161.1, XP_{—}003071804.1, EFW16963.1, XP_{—}002542118.1, XP_{—}001936677.1, EGD95268.1, XP_{—}003015678.1, XP_{—}501748.1, XP_{—}003169562.1, EFY96492.1, XP_{—}682653.1, XP_{—}002421356.1, CAK43439.1, EFY93677.1, XP_{—}747767.1, XP_{—}001244958.1, XP_{—}003019635.1, XP_{—}002847463.1, EGP83273.1, EGR52487.1, XP_{—}002622526.1, XP_{—}002563618.1, CBX99718.1, XP_{—}001552081.1, XP_{—}003066638.1, XP_{—}003176049.1, ACD75402.1, BAA05145.1, XP_{—}002482834.1, XP_{—}001257501.1, XP_{—}001934574.1, XP_{—}001269972.1, XP_{—}001587438.1, XP_{—}001215856.1, XP_{—}002149824.1, XP_{—}001550556.1, XP_{—}003011982.1, XP_{—}001827121.1, XP_{—}003233566.1, XP_{—}003022481.1, EGR47044.1, EFQ34695.1, XP_{—}003170005.1, BAG09241.1, XP_{—}002796370.1, XP_{—}003019300.1, XP_{—}002563873.1, CAK40654.1, EEH19741.1, XP_{—}003012518.1, EGD95716.1, XP_{—}003239409.1, BAJ04363.1, XP_{—}001537012.1, BAE66393.1, EGP85214.1, XP_{—}002487227.1, AAV66104.1, EGE07669.1, XP_{—}362943.2, XP_{—}003016806.1, EFQ27388.1, XP_{—}002384360.1, XP_{—}002836323.1, XP_{—}001274959.1, EFZ03093.1, XP_{—}661521.1, XP_{—}002849803.1, XP_{—}001589398.1, AAR99474.1, XP_{—}003189427.1, XP_{—}001823699.1, XP_{—}364111.1, XP_{—}001262753.1, EFY86805.1, XP_{—}001390153.2, XP_{—}002384738.1, XP_{—}001941811.1, XP_{—}001220831.1, XP_{—}003296981.1, XP_{—}002480829.1, BAD83681.1, XP_{—}001827526.2, XP_{—}369556.1, CAK38224.1, EFQ26532.1, XP_{—}002562328.1, XP_{—}001904540.1, EGO52476.1, XP_{—}002382002.1, XP_{—}001225874.1, XP_{—}958030.2, XP_{—}002540883.1, XP_{—}001908957.1, XP_{—}001559255.1, XP_{—}364102.1, EDP48064.1, XP_{—}365075.1, XP_{—}381460.1, CBX95930.1, XP_{—}003054099.1, XP_{—}361347.2, XP_{—}002846867.1, XP_{—}001214985.1, EFQ35175.1, XP_{—}002479062.1, XP_{—}001908613.1, XP_{—}003345380.1, EGR50567.1, XP_{—}002479350.1, XP_{—}001394417.2, XP_{—}001394159.2, XP_{—}002146776.1, EGP86783.1, EFX02953.1, CAK45889.1, XP_{—}003006887.1, XP_{—}002541427.1, XP_{—}750735.1, XP_{—}001257962.1, EGO51720.1, XP_{—}003005336.1, EGP83197.1, XP_{—}002149832.1, XP_{—}003052680.1, XP_{—}365851.1, XP_{—}001799910.1, XP_{—}003347175.1, XP_{—}002565258.1, EGR48918.1, EGR52524.1, XP_{—}964653.2, XP_{—}002147083.1, XP_{—}002843935.1, EEH19393.1, CAC10088.1, EEH47609.1, EEQ92528.1, XP_{—}001246560.1, XP_{—}002626168.1, XP_{—}003024880.1, XP_{—}003169255.1, XP_{—}003013780.1, XP_{—}003235691.1, XP_{—}746816.1, EGD98483.1, XP_{—}001389925.2, XP_{—}002842817.1, XP_{—}002797278.1, ADK36666.1, XP_{—}003305469.1, XP_{—}001548471.1, XP_{—}001806478.1, EFQ34989.1, XP_{—}001552987.1, CAC24473.1, XP_{—}002541530.1, EEQ89262.1, XP_{—}001247332.1, XP_{—}003066043.1, EDP47672.1, XP_{—}002628451.1, XP_{—}001910644.1, EGR44510.1, EFQ36733.1, XP_{—}003052472.1, XP_{—}001393445.2, XP_{—}001522438.1, EGO04179.1, XP_{—}001397944.2, CAK49049.1, EFQ30109.1, XP_{—}001585052.1, EGO30123.1, XP_{—}388496.1, XP_{—}003173913.1, CBF76609.1, XP_{—}003028593.1, EGO04180.1, CAK46976.1, XP_{—}370476.1, XP_{—}002145942.1, XP_{—}003004457.1, ADK36663.1, XP_{—}003040708.1, XP_{—}003351473.1, EFY84692.1, XP_{—}748328.2, XP_{—}003190325.1, XP_{—}002378813.1, EGR46513.1, XP_{—}003033448.1, XP_{—}002145326.1, XP_{—}662462.1, XP_{—}747469.1, XP_{—}001935085.1, EGR45892.1, EGO01601.1, EGP89995.1, XP_{—}001222615.1, XP_{—}001224356.1, EGN93507.1, XP_{—}001934479.1, BAK09464.1, EGO30124.1, XP_{—}001267956.1, ADK36661.1, EFY97845.1, XP_{—}001834501.1, EGO03790.1, XP_{—}001884320.1, XP_{—}003028899.1, AAP79879.1, EFY84206.1, BAK09467.1, XP_{—}003030469.1, XP_{—}001412594.1, XP_{—}001834508.1, XP_{—}001839436.2, XP_{—}002583529.1, XP_{—}001886288.1, XP_{—}002843371.1, XP_{—}001587730.1, BAK09418.1, BAK09442.1, EGO28830.1, EGE03365.1, EFZ01428.1, EGO03065.1, XP_{—}001558890.1, XP_{—}002487181.1, EGO29652.1, AAX49400.1, EFY92529.1, XP_{—}002380252.1, XP_{—}001884460.1, BAK09387.1, XP_{—}001839366.2, XP_{—}003031835.1, EFY99978.1, AAL67906.1, BAG09240.1, XP_{—}002381768.1, XP_{—}001800031.1, XP_{—}001825073.2, BAE63940.1, XP_{—}003028894.1, AAL67905.1, XP_{—}002910303.1, EGO22856.1, XP_{—}003028896.1, XP_{—}681680.1, XP_{—}002486603.1, XP_{—}001838945.2, EGR50064.1, XP_{—}001884349.1, XP_{—}001883816.1, CAK37996.1, CAO91865.1, XP_{—}003031227.1, XP_{—}001258702.1, XP_{—}001586739.1, XP_{—}001560806.1, CBF69707.1, ADN43682.1, XP_{—}001593179.1, XP_{—}001886909.1, XP_{—}001934479.1, XP_{—}001587730.1, XP_{—}001886909.1, XP_{—}001831709.2, XP_{—}001392650.1, XP_{—}366716.2, CAL69594.1, XP_{—}001269140.1, XP_{—}002566307.1, XP_{—}001555473.1, XP_{—}663925.1, XP_{—}001598033.1, XP_{—}001835239.2, EGN97256.1, XP_{—}001554305.1, NP_{—}182075.1, XP_{—}001560475.1, EFQ32286.1, XP_{—}001216788.1, XP_{—}002483975.1, AAC31835.1, NP_{—}850427.1, XP_{—}002143660.1, XP_{—}003327130.1, BAJ78287.1, XP_{—}002880182.1, ACB59278.1, EFQ36688.1, BAJ78285.1, BAJ78286.1, XP_{—}001798699.1, EEH44101.1, BAJ78288.1, BAJ78284.1, EGG02425.1, EGG03011.1, AAA34334.1, NP_{—}001189747.1, EGG02601.1, XP_{—}002978645.1, EGG11203.1, XP_{—}762610.1, XP_{—}762620.1, XP_{—}001545581.1, CAB44684.1, CAN80536.1, AAN05337.1, NP_{—}001049423.1, XP_{—}001791898.1, NP_{—}001031814.1, XP_{—}002279531.1, ABK94777.1, AAZ39646.1, XP_{—}002880183.1, ABC68403.1, XP_{—}002839066.1, EGG03014.1, XP_{—}002320074.1, NP_{—}001182854.1, CBI38795.3, XP_{—}002310605.1, NP_{—}196442.2, XP_{—}002270594.1, ABZ80830.1, XP_{—}002275905.1, CBI38796.3, XP_{—}002476978.1, CAB93726.1, EGG03624.1, EGG06527.1, NP_{—}197710.1, XP_{—}001768338.1, XP_{—}002270673.1, BAJ86572.1, XP_{—}002275806.1, CBI38797.3, XP_{—}002320072.1, CAN60189.1, XP_{—}002986290.1, XP_{—}002465888.1, CAN80040.1, XP_{—}002336104.1, XP_{—}002988354.1, XP_{—}002264277.1, EGD72898.1, XP_{—}002866853.1, EAY95236.1, XP_{—}002979701.1, XP_{—}002988762.1, XP_{—}002304502.1, XP_{—}002873349.1, XP_{—}003192947.1, CAN63571.1, NP_{—}001053615.1, NP_{—}176558.1, EGC49561.1, EGG09027.1, XP_{—}002314581.1, XP_{—}002446966.1, XP_{—}002320802.1, ABC59095.1, XP_{—}003323121.1, XP_{—}002974639.1, XP_{—}002395587.1, XP_{—}002866852.1, XP_{—}002319770.1, NP_{—}001146262.1, NP_{—}001169224.1, AAM65207.1, XP_{—}002529058.1, XP_{—}002886391.1, XP_{—}002320071.1, XP_{—}002446967.1, XP_{—}757870.1, EAY95147.1, XP_{—}002899664.1, EEH05830.1, XP_{—}002874114.1, ADO24345.1, BAJ88802.1, BAA05146.1, XP_{—}002963351.1, EAY88475.1, NP_{—}195658.3, XP_{—}002976944.1, ABC59093.1, XP_{—}002275114.1, XP_{—}003328407.1, CAN75428.1, BAJ86471.1, XP_{—}002981144.1, XP_{—}002277006.1, EAZ26110.1, ACN41008.1, XP_{—}002899542.1, XP_{—}001781614.1, EAY76187.1, BAK06758.1, XP_{—}002511745.1, XP_{—}002982626.1, XP_{—}002963763.1, NP_{—}001065111.1, ABF93892.1, XP_{—}002314117.1, BAK06287.1, XP_{—}001745327.1, NP_{—}001047674.1, XP_{—}002878665.1, XP_{—}002974847.1, NP_{—}179899.1, CAN80156.1, NP_{—}001053543.1, ABC59094.1, XP_{—}002328165.1, XP_{—}002270628.1, XP_{—}002275115.1, XP_{—}002980688.1, XP_{—}002465039.1, AAL91155.1, NP_{—}195910.1, XP_{—}002509820.1, NP_{—}200694.1, CAA62082.1, AAL75903.1, XP_{—}002468241.1, XP_{—}002883546.1, XP_{—}002862636.1, XP_{—}002312905.1, EAY79269.1, AAM12494.1, XP_{—}002875027.1, XP_{—}758010.1, XP_{—}002509524.1, AAP54707.2, XP_{—}002869292.1, NP_{—}001143079.1, ACF82946.1, XP_{—}002270497.1, XP_{—}002979685.1, XP_{—}002465041.1, XP_{—}002533544.1, AAG17470.1, XP_{—}002985393.1, NP_{—}191946.1, XP_{—}002525608.1, AAZ39642.1, XP_{—}002270428.1, XP_{—}002529227.1, CBI24485.3, XP_{—}001763206.1, EGG02922.1, XP_{—}002974848.1, NP_{—}001141467.1, CBI27149.3, NP_{—}001130907.1, XP_{—}002982474.1, NP_{—}001048917.1, XP_{—}002465889.1, ABZ80831.1, XP_{—}002464461.1, EAY88476.1, BAJ90714.1, XP_{—}002893825.1, ACN28568.1, XP_{—}002452782.1, XP_{—}002280004.1, XP_{—}001764611.1, NP_{—}001183394.1, BAJ89570.1, CBI24484.3, BAJ88840.1, ACG38359.1, CAN77648.1, BAJ91452.1, NP_{—}001141345.1, XP_{—}002282185.1, XP_{—}002980994.1, XP_{—}002299820.1, BAJ87982.1, BAJ91842.1, XP_{—}003325270.1, XP_{—}001760399.1, CBI34058.3, ADG34845.1, XP_{—}002523775.1, EEH21852.1, Q50EK3.1, BAK06748.1, XP_{—}002963764.1, ACN34158.1, XP_{—}001764503.1, XP_{—}002311750.1, XP_{—}001782495.1, XP_{—}002988642.1, XP_{—}002465625.1, XP_{—}002892051.1, XP_{—}002279649.1, NP_{—}171666.1, ABK28430.1, BAC42067.1, AED99869.1, NP_{—}174713.1, XP_{—}001781706.1, ABG66204.1, XP_{—}002964775.1, NP_{—}001064901.2, XP_{—}002961706.1, XP_{—}002519477.1, XP_{—}001559854.1, CBH32594.1, BAB92258.1, XP_{—}002264897.1, AAL59025.1, XP_{—}002862576.1, ACL53124.1, XP_{—}002521476.1, NP_{—}200045.1, BAJ89814.1, CBI38794.3, XP_{—}776769.1, NP_{—}001141372.1, EEC74485.1, EAY76557.1, XP_{—}002318861.1, NP_{—}001172660.1, XP_{—}002880978.1, AAO00706.1, BAK07606.1, XP_{—}002979336.1, BAC42841.1, BAF46296.1, XP_{—}002306380.1, XP_{—}002865907.1, ACG34921.1, XP_{—}002876375.1, NP_{—}001056685.1, XP_{—}002264292.1, XP_{—}002893443.1, NP_{—}001066096.1, EEE53477.1, CBH32607.1, EAY94753.1, NP_{—}001130939.1, NP_{—}182121.1, XP_{—}002437749.1, NP_{—}191222.1, XP_{—}002865881.1, XP_{—}569708.1, XP_{—}002279670.1, BAJ94774.1, ABF93894.1, BAD94304.1, ACG33785.1, NP_{—}194944.1, NP_{—}180337.1, AAB63277.1, BAJ85246.1, XP_{—}002456654.1, ACN27732.1, XP_{—}002445325.1, EER40289.1, XP_{—}001838184.2, BAJ85532.1, XP_{—}002866555.1, EAY88477.1, ACG47870.1, XP_{—}002310074.1, XP_{—}002457224.1, EAZ25521.1, BAJ87689.1, NP_{—}001044838.1, XP_{—}002521004.1, XP_{—}002882043.1, XP_{—}002527038.1, XP_{—}002318721.1, XP_{—}002979339.1, NP_{—}176086.1, XP_{—}001560028.1, ABC59092.1, ABF93891.1, ACR38435.1, EAY78983.1, NP_{—}179782.1, CCA21696.1, XP_{—}002334340.1, EFX88387.1, NP_{—}001044554.1, XP_{—}002321857.1, NP_{—}173862.1, NP_{—}195660.1, XP_{—}001554079.1, EAZ13864.1, EEC67630.1, EAY76183.1, AAP54710.2, NP_{—}001065112.2, ACD10924.1, XP_{—}001559275.1, EEC67338.1, XP_{—}002273811.1, ADJ68242.1, NP_{—}001065698.1, CAN66874.1, CAB41474.1, XP_{—}002868908.1, XP_{—}002904660.1, CAR47816.1, NP_{—}189243.1, EAY98229.1, XP_{—}002448320.1, O81117.2, XP_{—}002458797.1, XP_{—}002277129.1, BAJ88829.1, CAN67559.1, BAK08034.1, XP_{—}002894062.1, XP_{—}002894891.1, XP_{—}002279981.1, ABR16451.1, NP_{—}201150.1, AAM60854.1, XP_{—}002521002.1, XP_{—}002521474.1, XP_{—}002875311.1, NP_{—}195661.1, AAP79889.1, NP_{—}175193.1, P98188.1, BAK08270.1, CBI21357.3, XP_{—}002870817.1, XP_{—}002904451.1, ABA95812.1, XP_{—}002998647.1, NP_{—}001066166.2, XP_{—}002894690.1, EFY92064.1, XP_{—}002278009.1, XP_{—}002336002.1, CCA16508.1, XP_{—}002868909.1, EAZ31703.1, C96517, EAY86526.1, XP_{—}002307954.1, XP_{—}002904638.1, XP_{—}002266883.1, XP_{—}002439880.1, XP_{—}002892730.1, ADI52567.1, EGI61791.1, XP_{—}002511196.1, EGG04372.1, XP_{—}002511875.1, ACE75189.1, NP_{—}001055681.1, XP_{—}001589816.1, NP_{—}001170655.1, XP_{—}002300789.1, XP_{—}001934479.1, XP_{—}001587730.1, XP_{—}001554079.1, XP_{—}001559275.1, XP_{—}002868908.1, XP_{—}002998647.1, EFY92064.1, XP_{—}002605799.1, BAC43393.1, ABK28457.1, AAL54887.1, BAC43161.1, XP_{—}002333384.1, ZP_{—}03631129.1, AAL84318.1, BAJ99856.1, XP_{—}002593704.1, YP_{—}001965159.1, XP_{—}002454121.1, EFX88390.1, ABR16969.1, NP_{—}177109.3, XP_{—}002441724.1, NP_{—}001166017.1, BAB92256.1, ACE75340.1, AAZ39645.1, XP_{—}002312417.1, XP_{—}002887239.1, NP_{—}001172609.1, NP_{—}001065766.1, XP_{—}002515053.1, AAL54885.1, ABR16897.1, XP_{—}002878579.1, NP_{—}001140775.1, XP_{—}003275955.1, ZP_{—}08045694.1, BAJ94069.1, XP_{—}001654558.1, XP_{—}002436562.1, EAY88702.1, BAK03685.1, XP_{—}003327629.1, XP_{—}002322606.1, EEH42702.1, XP_{—}002037976.1, NP_{—}172774.1, XP_{—}002282477.1, EFX88388.1, XP_{—}002522465.1, EFZ21470.1, AAO41955.1, AAL54886.1, XP_{—}002450277.1, XP_{—}002862559.1, XP_{—}002335046.1, XP_{—}003328408.1, ACE75187.1, XP_{—}001849294.1, XP_{—}002444132.1, XP_{—}002894061.1, EFN77015.1, EGI69992.1, CBI17962.3, AAL54884.1, XP_{—}002998650.1, XP_{—}002105150.1, XP_{—}002877615.1, EFZ22412.1, XP_{—}002439815.1, XP_{—}002300790.1, CBI40391.3, AEI59774.1, XP_{—}002801151.1, XP_{—}003325267.1, XP_{—}001554577.1, EAY79865.1, XP_{—}002465796.1, XP_{—}002931035.1, ABA91371.1, ACE75338.1, XP_{—}001592850.1, XP_{—}001362981.1, XP_{—}002271246.1, EGB11905.1, NP_{—}176713.1, CBJ27248.1, NP_{—}566155.1, EFX87732.1, EEC71661.1, ACG29046.1, NP_{—}001130576.1, XP_{—}001843663.1, ABK25134.1, EGI65081.1, XP_{—}002722841.1, AAL67908.2, AAO15579.1, YP_{—}122047.1, EFA04617.1, YP_{—}001522424.1, ACB87383.1, NP_{—}001027517.1, EEE52725.1, XP_{—}002078257.1, XP_{—}002722842.1, ZP_{—}05128707.1, XP_{—}003208874.1, AAK31592.1, ABA95747.2, NP_{—}001181472.1, NP_{—}001075572.1, XP_{—}001108915.1, XP_{—}001520882.1, XP_{—}002063219.1, EFZ22408.1, AAL57721.1, EFW47740.1, AAQ20834.1, CAN74644.1, XP_{—}002722849.1, BAC30028.1, CAN75729.1, XP_{—}002115603.1, AAN72309.1, EEC68823.1, CAM18519.1, EAZ13863.1, XP_{—}002906159.1, NP_{—}001003947.1, ZP_{—}01858832.1, XP_{—}002882162.1, XP_{—}002089195.1, XP_{—}002892729.1, CAN68037.1, NP_{—}001130648.1, NP_{—}001166016.1, NP_{—}172773.4, ADJ68241.1, EGI62551.1, EFN63658.1, XP_{—}002300103.1, XP_{—}001658673.1, XP_{—}001367719.1, NP_{—}775146.1, XP_{—}001375048.1, AAH21377.1, NP_{—}727589.1, XP_{—}002271847.1, XP_{—}001809620.1, XP_{—}002897528.1, NP_{—}190421.1, XP_{—}002282468.1, XP_{—}536868.2, EEE58297.1, XP_{—}001992105.1, EAY82190.1, ADD20161.1, XP_{—}001363065.1, EAU77129.3, EAY72807.1, EGG03077.1, NP_{—}001181489.1, NP_{—}001177869.1, XP_{—}001966135.1, BAA99522.1, BAK07250.1, XP_{—}002133118.1, NP_{—}001042228.1, AAL57720.1, XP_{—}002897529.1, AAA35712.1, YP_{—}002275016.1, NP_{—}000770.2, XP_{—}002721578.1, XP_{—}321208.4, AAM09532.1, EFN61085.1, BAK06179.1, EFX88389.1, YP_{—}001602608.1, XP_{—}513140.3, NP_{—}001182438.1, AAD31068.1, NP_{—}001093242.1, XP_{—}001367758.2, EFZ18984.1, YP_{—}691921.1, CAH59968.1, AAS80270.1, CAH59967.1, ACQ99381.2, YP_{—}003810988.1, YP_{—}957888.1, CBW44755.1, ZP_{—}05042596.1, ZP_{—}01913735.1, ZP_{—}05043097.1, ADQ00145.1, YP_{—}004494060.1, ZP_{—}08206912.1, BAE78452.1, NP_{—}114222.1, ACZ56357.1, YP_{—}640381.1, ZP_{—}04384919.1, ZP_{—}08025219.1, ZP_{—}07715822.1, ZP_{—}06847816.1, YP_{—}001702784.1, AEK27137.1, ZP_{—}07716433.1, ZP_{—}08199554.1, YP_{—}004495520.1, YP_{—}345718.1, ZP_{—}08022914.1, YP_{—}001851443.1, BAG50428.1, YP_{—}001135848.1, BAF95905.1, YP_{—}345695.1, ACP39691.1, ACP39664.1, ACP39635.1, ACP39633.1, ACP39710.1, ACP39698.1, ACP39711.1, BAE47475.1, BAE47474.1, ABW76858.1, ACO50699.1, ACP39643.1, ACP39639.1, ACP39708.1, ACM68663.1, ACP39642.1, ACP39684.1, ACP39636.1, ZP_{—}05095005.1, ACP39652.1, BAE47473.1, ACM68664.1, ACP39646.1, ACP39680.1, ACP39692.1, ACP39675.1, ACP39632.1, ZP_{—}05129284.1, ACP39706.1, ACP39695.1, ACM68665.1, ACP39654.1, ACP39665.1, ACP39649.1, BAE47472.1, ACM68668.1, ACP39676.1, ACP39648.1, ACP39647.1, ZP_{—}01102434.1, ACM68666.1, ACP39641.1, ACM68669.1, ZP_{—}01625037.1, ACP39690.1, ACP39696.1, ACP39697.1, ACP39707.1, ACP39682.1, ACP39650.1, ACP39638.1, ZP_{—}05126641.1, CAH04396.1, ACP39658.1, ZP_{—}01102687.1, ACJ06772.1, YP_{—}001413041.1, YP_{—}552058.1, ADE05601.1, ADI19685.1, BAE47479.1, ZP_{—}01626700.1, ZP_{—}01618279.1, CAH61448.1, YP_{—}001411305.1, YP_{—}003591161.1, ZP_{—}01615522.1, ACM68667.1, ACP39651.1, ZP_{—}05095535.1, ZP_{—}01618489.1, NP_{—}418882.1, ADI19983.1, ACP39677.1, BAE47476.1, ACP39655.1, ACP39656.1, ADI19696.1, BAE47477.1, YP_{—}001413399.1, YP_{—}459878.1, BAE47480.1, BAE47481.1, ACP39653.1, BAE47478.1, YP_{—}001681656.1, ZP_{—}01618281.1, ZP_{—}01627262.1, YP_{—}001413057.1, YP_{—}760740.1, YP_{—}001242466.1, YP_{—}001203574.1, CAH61454.1, YP_{—}002129656.1, YP_{—}001672075.1, ACP39709.1, YP_{—}001990805.1, NP_{—}946959.1, YP_{—}001203575.1, YP_{—}783213.1, YP_{—}003059227.1, YP_{—}004110202.1, ACP39645.1, YP_{—}487538.1, CAH61451.1, YP_{—}570816.1, YP_{—}534107.1, YP_{—}001413223.1, YP_{—}001242465.1, YP_{—}557448.1, ZP_{—}08631162.1, NP_{—}773883.1, ZP_{—}00997728.1, ACP39683.1, NP_{—}768493.1, NP_{—}773882.1, ZP_{—}08271781.1, CAH61449.1, YP_{—}003883668.1, YP_{—}003332953.1, YP_{—}004535688.1, YP_{—}495502.1, YP_{—}459378.1, ZP_{—}08700267.1, ZP_{—}01863452.1, ZP_{—}06860085.1, BAE47487.1, YP_{—}617903.1, ZP_{—}08207422.1, BAE47486.1, ZP_{—}01041003.1, BAE47484.1, ACR78197.1, CAH61456.1, ZP_{—}01858113.1, ACP39681.1, BAE47485.1, ACP39673.1, BAE47483.1, ACP39669.1, BAE47482.1, ACP39674.1, ACP39704.1, ACP39703.1, YP_{—}497095.1, ACP39672.1, ACP39702.1, ACP39670.1, ACP39666.1, YP_{—}458852.1, ACP39687.1, ACP39688.1, ACP39634.1, ACP39686.1, ACP39660.1, ACP39700.1, YP_{—}001411309.1, ZP_{—}01465241.1, ACP39701.1, ACP39679.1, ACP39657.1, ACP39694.1, ACP39659.1, ACP39671.1, ACP39693.1 and YP_{—}003342921.1,
in particular
AAO73954.1, AAO73953.1, XP_{—}002546279.1, AAA34353.2, P30607.1, XP_{—}002421627.1, XP_{—}718670.1, CAA39366.1, XP_{—}001527524.1, AAO73955.1, AAO73956.1, XP_{—}002546278.1, EEQ43157.1, XP_{—}718669.1, AAA34354.1, P10615.3, XP_{—}002421628.1, 226487, P16141.3, CAA39367.1, Q9Y757.2, XP_{—}001485567.1, AAO73958.1, XP_{—}001383506.2, XP_{—}460111.2, AAO73959.1, Q12586.1, XP_{—}460112.2, AAO73960.1, Q12589.1, AAO73961.1, XP_{—}460110.2, EEQ43763.1, XP_{—}710174.1, EDK41572.2, XP_{—}001482650.1, CAA75058.1, XP_{—}002548818.1, Q12588.1, XP_{—}002422222.1, XP_{—}001383636.2, XP_{—}001525381.1, XP_{—}002548823.1, P30610.1, AAO73952.1, XP_{—}002548428.1, CAA36197.1, XP_{—}002421126.1, AAA34320.1, P16496.3, P30608.1, P24458.1, XP_{—}717999.1, XP_{—}001383817.1, Q9Y758.1, XP_{—}001482092.1, XP_{—}001383710.2, P30609.1, AAB24479.1, XP_{—}457792.1, XP_{—}001524144.1, XP_{—}457727.2, XP_{—}001525578.1, XP_{—}002616743.1, XP_{—}002614836.1, XP_{—}001525577.1, AAO73957.1, Q12585.1, XP_{—}001386440.2, XP_{—}002616857.1, XP_{—}001483276.1, XP_{—}500402.1, EDK39907.2, XP_{—}500560.1, XP_{—}001211376.1, XP_{—}002560027.1, XP_{—}504857.1, XP_{—}500855.1, XP_{—}504406.1, BAA31433.1, XP_{—}500856.1, XP_{—}501148.1, XP_{—}746567.1, XP_{—}001262425.1, XP_{—}001274843.1, XP_{—}002840588.1, XP_{—}002377641.1, XP_{—}001825995.1, XP_{—}001400739.1, XP_{—}718066.1, CAA35593.1, XP_{—}664735.1, XP_{—}002150795.1, XP_{—}500097.1, XP_{—}002483325.1, XP_{—}504311.1, XP_{—}500273.1, XP_{—}002548817.1, EDP54484.1, XP_{—}755288.1, XP_{—}001260447.1, EFY97851.1, ACD75398.1, ADK36660.1, XP_{—}001213081.1, XP_{—}002377989.1, XP_{—}001826299.1, XP_{—}001554811.1, XP_{—}501667.1, XP_{—}002148942.1, ADK36662.1, XP_{—}002565827.1, P30611.1, XP_{—}001267871.1, XP_{—}002372373.1, EFY84686.1, P43083.1, XP_{—}001263094.1, XP_{—}002148355.1, XP_{—}002568429.1, XP_{—}001817314.1, Q12587.1, XP_{—}001396435.1, XP_{—}001938589.1, XP_{—}001388497.2, XP_{—}663661.1, XP_{—}003295335.1, XP_{—}002152088.1, XP_{—}001212071.1, Q12573.1, XP_{—}002379858.1, XP_{—}001821592.1, XP_{—}002844341.1, XP_{—}001394678.1, ACD75400.1, XP_{—}003170343.1, XP_{—}001265480.1, XP_{—}002550661.1, EDP55514.1, XP_{—}001528842.1, XP_{—}749919.1, XP_{—}001593058.1, P30612.1, EGC48494.1, EEH04429.1, XP_{—}001585586.1, XP_{—}003236182.1, XP_{—}001400199.1, EEQ46951.1, XP_{—}721410.1, EGP87864.1, XP_{—}002380808.1, XP_{—}001792771.1, XP_{—}001208515.1, XP_{—}001216161.1, XP_{—}003071804.1, EFW16963.1, XP_{—}002542118.1, XP_{—}001936677.1, EGD95268.1, XP_{—}003015678.1, XP_{—}501748.1, XP_{—}003169562.1, EFY96492.1, XP_{—}682653.1, XP_{—}002421356.1, CAK43439.1, EFY93677.1, XP_{—}747767.1, XP_{—}001244958.1, XP_{—}003019635.1, XP_{—}002847463.1, EGP83273.1, EGR52487.1, XP_{—}002622526.1, XP_{—}002563618.1, CBX99718.1, XP_{—}001552081.1, XP_{—}003066638.1, XP_{—}003176049.1, ACD75402.1, BAA05145.1, XP_{—}002482834.1, XP_{—}001257501.1, XP_{—}001934574.1, XP_{—}001269972.1, XP_{—}001587438.1, XP_{—}001215856.1, XP_{—}002149824.1, XP_{—}001550556.1, XP_{—}003011982.1, XP_{—}001827121.1, XP_{—}003233566.1, XP_{—}003022481.1, EGR47044.1, EFQ34695.1, XP_{—}003170005.1, BAG09241.1, XP_{—}002796370.1, XP_{—}003019300.1, XP_{—}002563873.1, CAK40654.1, EEH19741.1, XP_{—}003012518.1, EGD95716.1, XP_{—}003239409.1, BAJ04363.1, XP_{—}001537012.1, BAE66393.1, EGP85214.1, XP_{—}002487227.1, AAV66104.1, EGE07669.1, XP_{—}362943.2, XP_{—}003016806.1, EFQ27388.1, XP_{—}002384360.1, XP_{—}002836323.1, XP_{—}001274959.1, EFZ03093.1, XP_{—}661521.1, XP_{—}002849803.1, XP_{—}001589398.1, AAR99474.1, XP_{—}003189427.1, XP_{—}001823699.1, XP_{—}364111.1, XP_{—}001262753.1, EFY86805.1, XP_{—}001390153.2, XP_{—}002384738.1, XP_{—}001941811.1, XP_{—}001220831.1, XP_{—}003296981.1, XP_{—}002480829.1, BAD83681.1, XP_{—}001827526.2, XP_{—}369556.1, CAK38224.1, EFQ26532.1, XP_{—}002562328.1, XP_{—}001904540.1, EGO52476.1, XP_{—}002382002.1, XP_{—}001225874.1, XP_{—}958030.2, XP_{—}002540883.1, XP_{—}001908957.1, XP_{—}001559255.1, XP_{—}364102.1, EDP48064.1, XP_{—}365075.1, XP_{—}381460.1, CBX95930.1, XP_{—}003054099.1, XP_{—}361347.2, XP_{—}002846867.1, XP_{—}001214985.1, EFQ35175.1, XP_{—}002479062.1, XP_{—}001908613.1, XP_{—}003345380.1, EGR50567.1, XP_{—}002479350.1, XP_{—}001394417.2, XP_{—}001394159.2, XP_{—}002146776.1, EGP86783.1, EFX02953.1, CAK45889.1, XP_{—}003006887.1, XP_{—}002541427.1, XP_{—}750735.1, XP_{—}001257962.1, EGO51720.1, XP_{—}003005336.1, EGP83197.1, XP_{—}002149832.1, XP_{—}003052680.1, XP_{—}365851.1, XP_{—}001799910.1, XP_{—}003347175.1, XP_{—}002565258.1, EGR48918.1, EGR52524.1, XP_{—}964653.2, XP_{—}002147083.1, XP_{—}002843935.1, EEH19393.1, CAC10088.1, EEH47609.1, EEQ92528.1, XP_{—}001246560.1, XP_{—}002626168.1, XP_{—}003024880.1, XP_{—}003169255.1, XP_{—}003013780.1, XP_{—}003235691.1, XP_{—}746816.1, EGD98483.1, XP_{—}001389925.2, XP_{—}002842817.1, XP_{—}002797278.1, ADK36666.1, XP_{—}003305469.1, XP_{—}001548471.1, XP_{—}001806478.1, EFQ34989.1, XP_{—}001552987.1, CAC24473.1, XP_{—}002541530.1, EEQ89262.1, XP_{—}001247332.1, XP_{—}003066043.1, EDP47672.1, XP_{—}002628451.1, XP_{—}001910644.1, EGR44510.1, EFQ36733.1, XP_{—}003052472.1, XP_{—}001393445.2, XP_{—}001522438.1, XP_{—}001397944.2, CAK49049.1, EFQ30109.1, XP_{—}001585052.1, XP_{—}388496.1, XP_{—}003173913.1, CBF76609.1, CAK46976.1, XP_{—}370476.1, XP_{—}002145942.1, XP_{—}003004457.1, ADK36663.1, XP_{—}003040708.1, XP_{—}003351473.1, EFY84692.1, XP_{—}748328.2, XP_{—}003190325.1, XP_{—}002378813.1, EGR46513.1, XP_{—}002145326.1, XP_{—}662462.1, XP_{—}747469.1, XP_{—}001935085.1, EGR45892.1, EGP89995.1, XP_{—}001222615.1, XP_{—}001224356.1, XP_{—}001934479.1, XP_{—}001267956.1, ADK36661.1, EFY97845.1, EFY84206.1, XP_{—}001412594.1, XP_{—}002583529.1, XP_{—}002843371.1, XP_{—}001587730.1, EGE03365.1, EFZ01428.1, XP_{—}001558890.1, XP_{—}002487181.1, EFY92529.1, XP_{—}002380252.1, EFY99978.1, BAG09240.1, XP_{—}002381768.1, XP_{—}001800031.1, XP_{—}001825073.2, BAE63940.1, XP_{—}681680.1, XP_{—}002486603.1, EGR50064.1, CAK37996.1, CAO91865.1, XP_{—}001258702.1, XP_{—}001586739.1, XP_{—}001560806.1, CBF69707.1, ADN43682.1, XP_{—}001593179.1, XP_{—}001392650.1, XP_{—}366716.2, CAL69594.1, XP_{—}001269140.1, XP_{—}002566307.1, XP_{—}001555473.1, XP_{—}663925.1, XP_{—}001598033.1, XP_{—}001554305.1, XP_{—}001560475.1, EFQ32286.1, XP_{—}001216788.1, XP_{—}002483975.1, XP_{—}002143660.1, EFQ36688.1, XP_{—}001798699.1, EEH44101.1, AAA34334.1, XP_{—}001545581.1, XP_{—}001791898.1, XP_{—}002839066.1, EGC49561.1, EEH05830.1, BAA05146.1, EEH21852.1, XP_{—}001559854.1, EER40289.1, XP_{—}001560028.1, XP_{—}001554079.1, XP_{—}001559275.1, EFY92064.1, XP_{—}001589816.1, EEH42702.1, XP_{—}001554577.1, XP_{—}001592850.1, YP_{—}691921.1, CAH59968.1, AAS80270.1, CAH59967.1, ACQ99381.2, YP_{—}003810988.1, YP_{—}957888.1, CBW44755.1, ZP_{—}05042596.1, ZP_{—}01913735.1, ZP_{—}05043097.1, ADQ00145.1, YP_{—}004494060.1, ZP_{—}08206912.1, BAE78452.1, NP_{—}114222.1, ACZ56357.1, YP_{—}640381.1, ZP_{—}04384919.1, ZP_{—}08025219.1, ZP_{—}07715822.1, ZP_{—}06847816.1, YP_{—}001702784.1, AEK27137.1, ZP_{—}07716433.1, ZP_{—}08199554.1, YP_{—}004495520.1, YP_{—}345718.1, ZP_{—}08022914.1, YP_{—}001851443.1, BAG50428.1, YP_{—}001135848.1, BAF95905.1, YP_{—}345695.1, ACP39691.1, ACP39664.1, ACP39635.1, ACP39633.1, ACP39710.1, ACP39698.1, ACP39711.1, BAE47475.1, BAE47474.1, ABW76858.1, ACO50699.1, ACP39643.1, ACP39639.1, ACP39708.1, ACM68663.1, ACP39642.1, ACP39684.1, ACP39636.1, ZP_{—}05095005.1, ACP39652.1, BAE47473.1, ACM68664.1, ACP39646.1, ACP39680.1, ACP39692.1, ACP39675.1, ACP39632.1, ZP_{—}05129284.1, ACP39706.1, ACP39695.1, ACM68665.1, ACP39654.1, ACP39665.1, ACP39649.1, BAE47472.1, ACM68668.1, ACP39676.1, ACP39648.1, ACP39647.1, ZP_{—}01102434.1, ACM68666.1, ACP39641.1, ACM68669.1, ZP_{—}01625037.1, ACP39690.1, ACP39696.1, ACP39697.1, ACP39707.1, ACP39682.1, ACP39650.1, ACP39638.1, ZP_{—}05126641.1, CAH04396.1, ACP39658.1, ZP_{—}01102687.1, ACJ06772.1, YP_{—}001413041.1, YP_{—}552058.1, ADE05601.1, ADI19685.1, BAE47479.1, ZP_{—}01626700.1, ZP_{—}01618279.1, CAH61448.1, YP_{—}001411305.1, YP_{—}003591161.1, ZP_{—}01615522.1, ACM68667.1, ACP39651.1, ZP_{—}05095535.1, ZP_{—}01618489.1, NP_{—}418882.1, ADI19983.1, ACP39677.1, BAE47476.1, ACP39655.1, ACP39656.1, ADI19696.1, BAE47477.1, YP_{—}001413399.1, YP_{—}459878.1, BAE47480.1, BAE47481.1, ACP39653.1, BAE47478.1, YP_{—}001681656.1, ZP_{—}01618281.1, ZP_{—}01627262.1, YP_{—}001413057.1, YP_{—}760740.1, YP_{—}001242466.1, YP_{—}001203574.1, CAH61454.1, YP_{—}002129656.1, YP_{—}001672075.1, ACP39709.1, YP_{—}001990805.1, NP_{—}946959.1, YP_{—}001203575.1, YP_{—}783213.1, YP_{—}003059227.1, YP_{—}004110202.1, ACP39645.1, YP_{—}487538.1, CAH61451.1, YP_{—}570816.1, YP_{—}534107.1, YP_{—}001413223.1, YP_{—}001242465.1, YP_{—}557448.1, ZP_{—}08631162.1, NP_{—}773883.1, ZP_{—}00997728.1
and particularly preferably
AAO73954.1, AAO73953.1, XP_{—}002546279.1, AAA34353.2, P30607.1, XP_{—}002421627.1, XP_{—}718670.1, CAA39366.1, AAO73955.1, AAO73956.1, XP_{—}002546278.1, EEQ43157.1, XP_{—}718669.1, AAA34354.1, P10615.3, XP_{—}002421628.1, 226487, P16141.3, CAA39367.1, AAO73958.1, AAO73959.1, Q12586.1, AAO73960.1, Q12589.1, AAO73961.1, EEQ43763.1, XP_{—}710174.1, CAA75058.1, XP_{—}002548818.1, Q12588.1, XP_{—}002422222.1, XP_{—}002548823.1, P30610.1, AAO73952.1, XP_{—}002548428.1, CAA36197.1, XP_{—}002421126.1, AAA34320.1, P16496.3, P30608.1, P24458.1, XP_{—}717999.1, P30609.1, AAB24479.1, AAO73957.1, Q12585.1, XP_{—}718066.1, CAA35593.1, XP_{—}002548817.1, P30611.1, P43083.1, Q12587.1, Q12573.1, XP_{—}002550661.1, P30612.1, EEQ46951.1, XP_{—}721410.1, XP_{—}002421356.1, BAA05145.1, BAG09241.1, CAC24473.1, BAG09240.1, AAA34334.1, BAA05146.1, XP_{—}500402.1, XP_{—}500560.1, XP_{—}504857.1, XP_{—}500855.1, XP_{—}504406.1, BAA31433.1, XP_{—}500856.1, XP_{—}501148.1, XP_{—}500097.1, XP_{—}504311.1, XP_{—}500273.1, XP_{—}501667.1, XP_{—}501748.1, YP_{—}691921.1, CAH59968.1, AAS80270.1, CAH59967.1, ACQ99381.2, YP_{—}003810988.1, YP_{—}957888.1, CBW44755.1, ZP_{—}05042596.1, ZP_{—}01913735.1, ZP_{—}05043097.1, ADQ00145.1, YP_{—}004494060.1, ZP_{—}08206912.1, BAE78452.1, NP_{—}114222.1, ACZ56357.1, YP_{—}640381.1, ZP_{—}04384919.1, ZP_{—}08025219.1, ZP_{—}07715822.1, ZP_{—}06847816.1, YP_{—}001702784.1, AEK27137.1, ZP_{—}07716433.1, ZP_{—}08199554.1, YP_{—}004495520.1, YP_{—}345718.1, ZP_{—}08022914.1, YP_{—}001851443.1, BAG50428.1, YP_{—}001135848.1, BAF95905.1, YP_{—}345695.1, ACP39691.1, ACP39664.1, ACP39635.1, ACP39633.1, ACP39710.1, ACP39698.1, ACP39711.1, BAE47475.1, BAE47474.1, ABW76858.1, ACO50699.1, ACP39643.1, ACP39639.1, ACP39708.1, ACM68663.1, ACP39642.1, ACP39684.1, ACP39636.1, ZP_{—}05095005.1, ACP39652.1, BAE47473.1, ACM68664.1, ACP39646.1, ACP39680.1, ACP39692.1, ACP39675.1, ACP39632.1, ZP_{—}05129284.1, ACP39706.1, ACP39695.1, ACM68665.1, ACP39654.1, ACP39665.1, ACP39649.1, BAE47472.1, ACM68668.1, ACP39676.1, ACP39648.1, ACP39647.1, ZP_{—}01102434.1, ACM68666.1, ACP39641.1, ACM68669.1, ZP_{—}01625037.1, ACP39690.1, ACP39696.1, ACP39697.1, ACP39707.1, ACP39682.1, ACP39650.1 and ACP39638.1 and
proteins with a polypeptide sequence wherein up to 60%, preferably up to 25%, particularly preferably up to 15%, in particular up to 10, 9, 8, 7, 6, 5, 4, 3, 2 or 1% of the amino acid residues are modified compared to the aforementioned reference sequences by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E₁ₐ in general is understood to mean in particular the conversion of lauric acid and/or methyl laurate to ω-hydroxylauric acid and/or methyl ω-hydroxylaurate.

Modifications of amino acid residues of a given polypeptide sequence which lead to no significant modifications of the properties and function of the given polypeptide are known to those skilled in the art. Thus for example many amino acids can often be exchanged for one another without problems; examples of such suitable amino acid substitutions are: Ala by Ser; Arg by Lys; Asn by Gln or His; Asp by Glu; Cys by Ser; Gln by Asn; Glu by Asp; Gly by Pro; His by Asn or Gln; Ile by Leu or Val; Leu by Met or Val; Lys by Arg or Gln or Glu; Met by Leu or Ile; Phe by Met or Leu or Tyr; Ser by Thr; Thr by Ser; Trp by Tyr; Tyr by Trp or Phe; Val by Ile or Leu. It is also known that modifications, particularly at the N- or C-terminus of a polypeptide in the form of for example amino acid insertions or deletions, often exert no significant influence on the function of the polypeptide.

AlkB alkane hydroxylases preferred according to the invention are selected from the list

YP_{—}001185946.1, Q9WWW6.1, YP_{—}957898.1, YP_{—}957728.1, YP_{—}694427.1, BAC98365.1, ZP_{—}00957064.1, CAC86944.1, YP_{—}001672212.1, CAB59525.1, ACH99213.1, ACH99215.1, ACH99216.1, AAK56792.1, ACH99229.1, ACS91348.1, AAP41820.1, ZP_{—}05128075.1, CAM58121.1, CAM58085.1, ACQ44675.1, ACZ62808.1, ZP_{—}01738706.1, ZP_{—}01916228.1, ZP_{—}01225325.1, YP_{—}001023605.1, ACJ22747.1, ACT91140.1, AAT91722.2, CBA27418.1, YP_{—}001889129.1, EGC97932.1, ACT91201.1, ZP_{—}05083049.1, YP_{—}554098.1, ZP_{—}01900149.1, ADG26619.1, ADG26657.1, ADG26640.1, ZP_{—}06838771.1, ADG26649.1, ADG26651.1, ZP_{—}02374120.1, YP_{—}368326.1, ZP_{—}02380481.1, ADG26643.1, ADG26628.1, YP_{—}442346.1, ADG26620.1, ADG26647.1, ZP_{—}07673680.1, ADG26638.1, YP_{—}002232139.1, YP_{—}001118743.1, ZP_{—}01764629.1, YP_{—}108945.1, YP_{—}334185.1, ZP_{—}04897834.1, ZP_{—}02889567.1, YP_{—}620386.1, YP_{—}002897546.1, ZP_{—}02166109.1, ZP_{—}02904755.1, ADG26639.1, YP_{—}001892637.1, ADG26642.1, ZP_{—}04939380.1, ZP_{—}02464124.1, YP_{—}102417.1, CAC36356.1, ACJ22727.1, YP_{—}001764240.1, YP_{—}002765609.1, YP_{—}001945311.1, ZP_{—}03586616.1, ACJ22665.1, ZP_{—}03574223.1, CAC37038.1, ZP_{—}02456517.1, YP_{—}001807560.1, YP_{—}002779449.1, AAK97454.1, YP_{—}002912304.1, ACR55689.1, YP_{—}003397515.1, YP_{—}004361423.1, YP_{—}772734.1, ACJ65014.1, ACT31523.1, ACJ22750.1, ZP_{—}07375042.1, YP_{—}002776786.1, ACB11552.1, ZP_{—}02363472.1, ADG26653.1, ZP_{—}04383196.1, ZP_{—}02356342.1, ACJ22751.1, YP_{—}952571.1, ACU43494.1, YP_{—}001135977.1, YP_{—}002764193.1, YP_{—}003855036.1, YP_{—}004078475.1, AAK97448.1, ZP_{—}04388098.1, ACX30747.1, ADG26632.1, ACJ22719.1, ADO21492.1, ZP_{—}05061580.1, ADR72654.1, ACZ65961.1, ACX30755.1, YP_{—}001849604.1, AAV64895.1, YP_{—}004495037.1, YP_{—}702497.1, YP_{—}001069662.1, ZP_{—}06850622.1, BAF34299.1, CAB51024.2, YP_{—}004008018.1, YP_{—}003768535.1, ACJ65013.1, ZP_{—}07282765.1, YP_{—}886209.1, ACJ22725.1, ZP_{—}08155372.1, YP_{—}004493362.1, ZP_{—}05228000.1, ZP_{—}07717360.1, BAD67020.1, YP_{—}004524245.1, ZP_{—}07715778.1, NP_{—}217769.1, ACS91349.1, YP_{—}960105.1, ZP_{—}07014137.1, YP_{—}004746682.1, ZP_{—}08022271.1, ACN62569.1, ADQ37951.1, YP_{—}003647687.1, YP_{—}003837040.1, ADG26600.1, YP_{—}002768905.1, ZP_{—}08553310.1, ADG26597.1, ACJ22749.1, ADG26598.1, YP_{—}001704327.1, ZP_{—}04385381.1, ZP_{—}04751264.1, ADG26609.1, ADG26610.1, ZP_{—}06417258.1, ADG26607.1, ADP98338.1, YP_{—}003275257.1, YP_{—}004084103.1, ADG26630.1, ADG26625.1, ADG26605.1, ADG26599.1, ZP_{—}05218167.1, ADQ37950.1, YP_{—}921354.1, ADG26645.1, ADG26612.1, YP_{—}004493370.1, YP_{—}638501.1, YP_{—}003809668.1, NP_{—}962298.1, ZP_{—}04750514.1, ADG26608.1, ADT82701.1, ACJ06773.1, YP_{—}120833.1, ADG26618.1, ADG26602.1, ADG26623.1, ZP_{—}04383566.1, ZP_{—}08122407.1, YP_{—}004077166.1, ZP_{—}05041651.1, ZP_{—}04608296.1, ABU93351.2, YP_{—}003658078.1, ADQ37949.1, ADG26652.1, YP_{—}002765850.1, AAK97447.1, CAD24434.1, CAC40954.1, ACT91203.1, YP_{—}120829.1, ZP_{—}07282558.1, YP_{—}003298195.1, YP_{—}001851790.1, ZP_{—}05827357.1, ADG26633.1, CAB51020.1, YP_{—}953908.1, ZP_{—}07990416.1, YP_{—}119532.1, ZP_{—}08442348.1, ZP_{—}08276444.1, ZP_{—}04661203.1, ABO12068.2, YP_{—}001846325.1, ADQ37952.1, ZP_{—}08198697.1, ZP_{—}00996652.1, YP_{—}001707231.1, ZP_{—}08433663.1, ZP_{—}08205256.1, YP_{—}003732372.1, YP_{—}906529.1, ACT91204.1, YP_{—}001506534.1, YP_{—}001713880.1, YP_{—}883357.1, YP_{—}004525252.1, ADG26604.1, YP_{—}001134633.1, ZP_{—}08195602.1, ZP_{—}06690500.1, ZP_{—}05826167.1, ADY81595.1, ZP_{—}06056754.1, AAK31348.1, YP_{—}251715.1, ZP_{—}08461977.1, ZP_{—}05847237.1, YP_{—}712218.1, YP_{—}001084670.1, ZP_{—}04387164.1, YP_{—}260041.1, YP_{—}002873097.1, ADG26614.1, AAK97446.1, YP_{—}001280943.1, ZP_{—}04386125.1, AAC36353.2, CCA29159.1, CAD10804.1, CCA29151.1, CAC40953.1, CCA29161.1, ABA55770.1, AAS93604.4, CCA29173.1, CCA29155.1, CCA29156.1, ABA55772.1, CCA29154.1, ABA55793.1, CCA29162.1, CCA29170.1, ZP_{—}03824539.1, CCA29166.1, CCA29136.1, ZP_{—}06065934.1, ABB54493.1, CCA29169.1, YP_{—}003112137.1, CCA29127.1, CCA29148.1, CCA29160.1, ZP_{—}06057458.1, ABA55773.1, YP_{—}004016090.1, CCA29139.1, YP_{—}480358.1, ABA55787.1, CCA29150.1, CCA29130.1, ZP_{—}07775830.1, ABA55779.1, CCA29132.1, YP_{—}003732938.1, BAB33284.1, CCA29149.1, CCA29145.1, ABA55783.1, CCA29137.1, CCA29129.1, CCA29158.1, CCA29176.1, CCA29142.1, CCA29144.1, BAB33287.1, CCA29133.1, CCA29140.1, CCA29135.1, ZP_{—}06066074.1, ZP_{—}03823182.1, CCA29171.1, CCA29152.1, CCA29131.1, ABA55780.1, CCA29163.1, CCA29143.1, CCA29153.1, YP_{—}001580600.1, CCA29134.1, CCA29138.1, YP_{—}046098.1, ZP_{—}06072466.1, ZP_{—}05361594.1, ACU43504.1, CCA29147.1, CCA29146.1, ZP_{—}06061712.1, ACT91185.1, ACT91147.1, ACT91178.1, ACT91167.1, ACT91181.1, ACT91188.1, ZP_{—}06069784.1, ACT91205.1, ZP_{—}06725872.1, ACT91171.1, CCA29128.1, ABY56787.1, ADE05602.1, ACU43474.1, ACJ22718.1, ABB90688.1, ACU43519.1, ABB96093.1, ACU43485.1, ACU43493.1, ABW76857.1, ACT91163.1, ACJ22673.1, ZP_{—}06188150.1, ACT91242.1, ACT91225.1, ACT91211.1, ACU43479.1, ACU43491.1, ACU43522.1, ACU43486.1, ACT91221.1, ACJ22662.1, ACU43506.1, ACU43487.1, ACT91259.1, AAA97866.1, ACU43502.1, YP_{—}001252544.1, ABB96084.1, ACU43520.1, ACJ22668.1, ACU43503.1, ACT91230.1, ABA55777.1, ACT91231.1, ZP_{—}01748311.1, ACJ22724.1, ACU43475.1, ACU43511.1, ACU43490.1, ZP_{—}08330953.1, ACU43484.1, CBX01596.1, ACT91168.1, YP_{—}096989.1, ACT91215.1, YP_{—}125370.1, ACT91233.1, ACU43478.1, ADE05603.1, ACJ22715.1, ACU43512.1, ACT91196.1, ACJ22692.1, ACU43510.1, ACU43521.1, ACT91174.1, ACT91213.1, ACT91142.1, ACT91206.1, ACT91216.1, ACT91182.1, ACT91255.1, ACT91246.1, ACT91217.1, ACT91155.1, ACT91240.1, ACT91207.1, ACU43495.1, YP_{—}128249.1, ACT91160.1, YP_{—}004052990.1, ACT91226.1, ACU43507.1, ABO61855.1, ACT91214.1, ACT91220.1, YP_{—}001188237.1, ACJ22689.1, ZP_{—}01689499.1, YP_{—}004379711.1, ACJ22748.1, ABB90683.1, ACT91223.1, ACT91235.1, ABO61786.1, ACU43508.1, ACU43492.1, ACT91219.1, ACT91244.1, ABO61856.1, ACT91239.1, ACU43473.1, ABO61850.1, ACT91262.1, ACT91261.1, ACT91224.1, ACU43499.1, ACU43488.1, ADO21767.1, YP_{—}004654946.1, ADO21777.1, ABB96089.1, ABO61852.1, ABO61847.1, ACT91222.1, ADO21764.1, ACU43477.1, ADO21773.1, ABO61787.1, ABB96080.1, ABO61857.1, ACT91228.1, ABB96070.1, ADO21744.1, ACT91245.1, CAG17608.1, ADO21747.1, YP_{—}001349162.1, ABK63807.1, ZP_{—}06879583.1, NP_{—}250216.1, ACT91234.1, ZP_{—}01364874.1, ABO61789.1, ADO21772.1, ACU43516.1, ACU43505.1, ACU43501.1, ACT91236.1, ZP_{—}07792758.1, ACZ64723.1, ADO21743.1, ADO21759.1, ACZ64752.1, ADO21755.1, ACD75517.1, YP_{—}790621.1, ACB11551.1, ADO21748.1, NP_{—}251264.1, ZP_{—}01365940.1, ADO21762.1, ADO21739.1, ACU43496.1, ABO61854.1, ZP_{—}06878434.1, ACU43489.1, ACU43483.1, ADO21746.1, ACT91237.1, ZP_{—}01895378.1, ACT91164.1, ADO21736.1, ACJ22711.1, ACZ64754.1, ZP_{—}05042146.1, ADO21688.1, ADO21648.1, YP_{—}001348003.1, ADP98656.1, ADO21737.1, ADO21760.1, ADO21754.1, ADO21740.1, ACZ64758.1, ACU43497.1, ZP_{—}01912185.1, ABB96111.1, ACU43482.1, ACB11549.1, ADO21775.1, CCA29157.1, ADO21681.1, ADO21668.1, ADO21656.1, ACU43517.1, ACT91165.1, ACJ22695.1, ACJ22688.1, ABB96071.1, ADO21763.1, ACT91241.1, ADO21735.1, ACB11550.1, ADO21778.1, ACT91172.1, ADO21765.1, ABB96087.1, CBJ30233.1, ACJ22752.1, ABB96105.1, ACB15251.1, ACJ22694.1, ACZ64741.1, ACZ64706.1, ABB96108.1, ACT91191.1, ABB96101.1, ABB90691.1, ACZ64745.1, YP_{—}691842.1, ABB96075.1, ABB90682.1, ABB90690.1, ADO21676.1, ADO21679.1, ABO61768.1, YP_{—}435857.1, ACJ22722.1, ACT91238.1, ACZ64725.1, CAC14062.1, ADO21682.1, ACZ64771.1, ACZ64718.1, ACZ64724.1, ADO21670.1, ADO21667.1, CAC37048.1, ACZ64708.1, ABB96092.1, ACJ22687.1, ACZ64703.1, ADO21690.1, ABB92364.1, ACB11547.1, ACZ64720.1, ADO21655.1, ACZ64717.1, ADO21680.1, ACZ64757.1, ACZ64733.1, ACT91144.1, ACU43481.1, ACT91179.1, ZP_{—}02181409.1, ACZ64704.1, ABB96073.1, ACJ22675.1, ACZ64721.1, ABB96090.1, ACJ22729.1, ACU43515.1, ZP_{—}01307000.1, ABB90685.1, YP_{—}003862088.1, ACZ64715.1, ACZ64710.1, ACJ22735.1, ABB90687.1, ADO21661.1, ADO21674.1, ACT91177.1, ABB54492.1, ABB96076.1, ABB92365.1, ACT91194.1, ADO21689.1, ACJ22691.1, ABB90681.1, ADO21649.1, ADO21671.1, ACZ64728.1, ABB96095.1, CAC40945.1, ADO21652.1, ADO21665.1, ADE08461.1, ADO21678.1, ACZ64705.1, ACJ22690.1, ADO21675.1, ADO21685.1, ABB96072.1, ACJ22736.1, ACB11540.1, ABB96091.1, ACI04540.1, ACT91251.1, ACT91146.1, ACT91166.1, ACT91156.1, ADO21752.1, ADO21673.1, ADO21725.1, ABB96104.1, ABB90694.1, ABB90696.1, ACT91173.1, ADO21647.1, ZP_{—}03700804.1, ACT91232.1, ADO21694.1, CAC40949.1, ABB92361.1, ACT91195.1, ACI04538.1, ADO21691.1, ACJ22685.1, ADO21653.1, ABS12461.1, ACZ64736.1, ACZ64772.1, ABB90680.1, ADO21659.1, ACZ64774.1, ADO21684.1, ADO21729.1, ADO21650.1, ADO21733.1, ACZ64755.1, ACZ64751.1, ABA55775.1, ADO21738.1, CCA29174.1, ADO21669.1, ACZ64744.1, ADO21654.1, ADO21768.1, ABB96106.1, CCA29168.1, ACT91176.1, ACB11555.1, ABB90695.1, ADO21660.1, ACJ22666.1, ACZ64778.1, ADO21766.1, ADO21677.1, ZP_{—}02161687.1, CCA29165.1, ADO21745.1, ACB11548.1, ABB90689.1, ABB96107.1, AAT46052.1, ADO21718.1, ADO21722.1, ABB96088.1, EFW40271.1, ADO21686.1, ABB96103.1, ACU43500.1, ACB11536.1, ABB92360.1, CCA29167.1, ACT91199.1, ACZ64770.1, ACJ22716.1, ABA55786.1, ACZ64737.1, ABB96083.1, ACJ22676.1, ACZ64735.1, ACT91212.1, ACJ22765.1, CAJ01371.1, CAC17734.1, ABD36389.1, ACB11537.1, CAC08515.1, ACZ64714.1, ACU43513.1, ABB96082.1, ADN21387.1, ADO21711.1, ABD36392.1, ABR10770.1, CAC37049.1, ABB96098.1, ABB90692.1, ACB11535.1, ACZ64768.1, ACJ22756.1, ABB96094.1, ABA55791.1, ABB96078.1, ACT91141.1, ACZ64779.1, ACZ64750.1, CAJ01370.1, ACZ64753.1, ACU43480.1, ABA55794.1, ABB96085.1, ABB96110.1, YP_{—}004448035.1, ACZ64709.1, ABB96102.1, ACZ64773.1, CCA29175.1, ACZ64749.1, ACZ64756.1, ACZ64781.1, ABO61777.1, ACZ64759.1, ACZ64764.1, ACZ64740.1, ACT91249.1, ZP_{—}03702922.1, ACB11545.1, ACZ64775.1, ACZ64769.1, ACT91145.1, ACZ64742.1, ACT91254.1, ACZ64762.1, ACZ64716.1, ACZ64777.1, ADM26559.1, ABB96096.1, ACZ64780.1, ZP_{—}01201250.1, CAH55829.1, ZP_{—}01052921.1, ABB96077.1, ADO21658.1, ACT91161.1, ABB90684.1, ACR56750.1, ABB90697.1, ACZ64746.1, ABB92367.1, ACT91139.1, ACZ64763.1, ACT91200.1, ABO61773.1, ABB96081.1, ACZ64748.1, ACZ64782.1, ACU43498.1, ADO21651.1, ABB90679.1, BAG06233.1, ACZ64747.1, ABB96086.1, ACZ64761.1, ABB92370.1, ABO61774.1, ACT91175.1, ABB90686.1, ACB11546.1, ZP_{—}01740604.1, ABO61785.1, YP_{—}001531377.1, XP_{—}001434539.1, ABA55767.1, ABO21865.1, ABF55636.1, ABA55751.1, ABB90698.1, ADD12311.1, ACZ64765.1, ABB92366.1, ABB92368.1, ACI04539.1, XP_{—}001023288.1, ACZ64783.1, ADO21692.1, ZP_{—}01753800.1, ACZ64760.1, ACZ64700.1, ZP_{—}01055480.1, ACZ64767.1, ACZ64701.1, ABA55745.1, ABA55752.1, ACZ64766.1, YP_{—}614640.1, ABA55759.1, ADO21723.1, BAG06232.1, ZP_{—}01002389.1, ABB90693.1, ACT91264.1, ABB92358.1, BAF99026.1, ABR10769.1, ZP_{—}00959618.1, AEA08580.1, ADD22986.1, CAB51023.1, CAC40958.1, ADO21709.1, CAB51025.1, ACI15226.1, ACJ22680.1, ZP_{—}05741459.1, ACT91248.1, ABU48567.1, ABO61792.1, ACJ22754.1, EFN53276.1, AAL87644.1, ACT91209.1, ZP_{—}02147281.1, ACU43518.1, ACZ64776.1, ACB11543.1, ACT91151.1, ACJ22764.1, ACT91159.1, ABA18186.1, AEA08579.1, ADO21770.1, ABF55634.1, CAA27179.1, ABA55741.1, ADO21705.1, ZP_{—}01754375.1, ACB11541.1, ACR56751.1, ACT91250.1, ADO21769.1, ADO21753.1, ABB96097.1, ACT91208.1, ABO21867.1, ADO21757.1, ACB11554.1, ABA55749.1, CAC40951.1, ADO21719.1, ABB96074.1, ZP_{—}00954267.1, ZP_{—}05786269.1, AEH76912.1, ABA55742.1, ABA55748.1, BAG06236.1, ADO21732.1, ABA55750.1, ABA55768.1, ACT31522.1, ZP_{—}05090796.1, ACZ64739.1, YP_{—}915886.1, ADO21731.1, CAC40948.1, XP_{—}001032273.1, AEH76911.1, ABA55743.1, ABO61769.1, ABA55755.1, ZP_{—}05122263.1, ADO21756.1, ABA55744.1, ABA55746.1, ZP_{—}01901011.1, ZP_{—}02150761.1, ADO21742.1, ACR56752.1, ABA55747.1, ABF55637.1, ABA55740.1, ABA55760.1, ZP_{—}00948812.1, ABA55804.1, ADO21771.1, ZP_{—}05342453.1, ABF55638.1, YP_{—}508336.1, ABB92357.1, ZP_{—}01049702.1, ABU48546.1, ABU48555.1, ABA55764.1, ABO21866.1, ZP_{—}05079274.1, ZP_{—}01880441.1, ACZ64738.1, ZP_{—}05842058.1, ACT91218.1, ABA55769.1, ABA55739.1, ABA55803.1, ACT91247.1, ABA55782.1, ACZ17539.1, ABB92359.1, ACH69966.1, ZP_{—}01035050.1, ACZ17537.1, ABA55774.1, ACZ64729.1, ACZ17538.1, ZP_{—}01751972.1, ACZ64731.1, ACZ64702.1, AAR13803.1, AEJ28400.1, ZP_{—}05099213.1, CAB51021.1, ACZ17531.1, AEH76914.1, ZP_{—}05051648.1, ACZ64726.1, ACZ17540.1, ACZ64727.1, ZP_{—}02152773.1, ACT91253.1, ACZ17536.1, XP_{—}001423873.1, ACZ17534.1, YP_{—}168645.1, ACZ17520.1, ABY56786.1, ACB11539.1, ZP_{—}01157350.1, AEH76910.1, ABY56784.1, AAY85982.1, ACT91257.1, ACB11544.1, ACZ17532.1, ZP_{—}01746661.1, ABA55771.1, BAG06235.1, EGR32049.1, YP_{—}001166282.1, ABO61799.1, ABA55757.1, AEH76915.1, ACO59264.1, ABO26125.1, AEA08577.1, ACT91265.1, ABY56785.1, ACZ17528.1, ABO61798.1, ADO21749.1, ACT91263.1, ACT91252.1, ACZ64722.1, ABO61771.1, ACZ17526.1, ABO26123.1, ADO21714.1, ZP_{—}01000906.1, ABO61796.1, ADC29534.1, ACB15250.1, ACD47155.1, ACZ17525.1, ACB11553.1, ABD36391.1, AEH76913.1, ACZ17523.1, ABO61781.1, ACZ17524.1, ZP_{—}01914093.1, ACB11538.1, ZP_{—}01015838.1, ACJ22693.1, ACB15252.1, CAC86945.1, ACO59265.1, ABO61791.1, ACZ17521.1, ABO26124.1, ACZ64732.1, ACU43514.1, ACT91256.1, ACM63043.1, ACS75820.1, ZP_{—}08666479.1, CAH03133.1, BAG06234.1, AEH76916.1, ABO61790.1, ABE72965.1, ACZ64711.1, ACB11542.1, AAY26148.1, ABA55776.1, ACZ17522.1, ACZ64734.1, AEA08578.1, ACZ17530.1, ZP_{—}04062748.1, ACJ22755.1, NP_{—}969039.1, AAY26149.1, ACJ22761.1, ABU48543.1, ZP_{—}08414255.1, AAT91720.1, ZP_{—}01444283.1, ABA55796.1, ABU48542.1, YP_{—}001042010.1, YP_{—}001234392.1, YP_{—}351510.1, ACZ64730.1, ZP_{—}08634611.1, ACZ17529.1, ACJ22667.1, AAT91719.1, YP_{—}004283531.1, ABO61801.1, ACZ17519.1, ABO15266.1, CAB51040.1, ACZ64707.1, ACJ22766.1, ABO26121.1, ZP_{—}01878984.1, CAB51039.1, ABA55795.1, ABO15269.1, ABO15247.1, ACJ22763.1, ABO15251.1, ACZ17527.1, ABO15270.1, ACJ22769.1, ADE06670.1, ZP_{—}05780387.1, ABO61770.1, ACT91258.1, ABO15258.1, ABO15257.1, ABU48545.1, CAC86946.1, ABO15267.1, ZP_{—}01741446.1, ABU48544.1, YP_{—}002296646.1, AEH76917.1, ADC29550.1, YP_{—}002527219.1, ABK88246.1, ADN21388.1, ACT91210.1, ZP_{—}05064795.1, ABJ16487.1, XP_{—}002675644.1, ABJ16489.1, ADA71089.1, ADA71088.1, AAT46053.1, ZP_{—}01744806.1, ZP_{—}01037964.1, ZP_{—}00955262.1, ABJ16493.1, YP_{—}001840157.1, ZP_{—}00964204.1, ABB40596.1, ACB15249.1, ADD82963.1, YP_{—}004499590.1, ZP_{—}01011524.1, ACJ22758.1, ZP_{—}01748906.1, ACV30052.1, ZP_{—}06191942.1, YP_{—}001188029.1, ACD63080.1, YP_{—}166583.1, AAV41375.1, ZP_{—}00998265.1, ACJ22757.1, ABB13506.2, ABI13999.1, ABI14004.1, ABB13509.1, YP_{—}371980.1, ZP_{—}01755711.1, ZP_{—}05065835.1, ZP_{—}00959368.1, XP_{—}001020063.1, ABJ16481.1, ABI14006.1, ZP_{—}05101918.1, ZP_{—}01913733.1, ABI14001.1, ABM92270.1, ABI14003.1, CAH03132.1, YP_{—}973211.1, ABA55797.1, YP_{—}003578527.1, ABJ16483.1, ABJ16482.1, CBY78068.1, ACT91260.1, YP_{—}509155.1, ABB13508.1, ABJ16485.1, ABO61779.1, ABI14005.1, ACM63042.1, ADC29543.1, ZP_{—}02153440.1, YP_{—}709335.1, ABI13998.1, ABI14002.1, AAB70825.1, ACX30751.1, ABI14000.1, YP_{—}003617173.1, ZP_{—}01155421.1, ACX30752.1, NP_{—}542887.1, ADC29546.1, AAC38359.1, ADC29541.1, XP_{—}001020064.1, ZP_{—}01442436.1, ZP_{—}05103090.1, ADC29544.1, ABO61809.1, AAY89939.1, ACH99235.1, CAH55830.1, ABO26095.1, YP_{—}004011670.1, ABO26084.1, ADA71083.1, ABO26087.1, ABO61806.1, ADC29531.1, ABO26109.1, ACJ22753.1, ABO26089.1, ABO26093.1, ABO26092.1, ABO61827.1, ABO26105.1, ABO26112.1, AAT91721.1, ABO26120.1, ABO26090.1, ABO26088.1, ABO61811.1, ABO61783.1, CAH55827.1, ACH99232.1, ABO61828.1, ADC29530.1, ACH99234.1, AAQ88276.1, CAH55823.1, ABO26103.1, ACH99233.1, ABO61836.1, ABO26094.1, ABO61840.1, YP_{—}004534277.1, ZP_{—}05845010.1, ABO61821.1, ACH99231.1, AAV68403.1, ABO61839.1, CAH56098.1, ABO26085.1, ABO61826.1, ABO61822.1, ABO26110.1, ABO61810.1, ABO61844.1, ABO61825.1, ABO26099.1, ACJ22767.1, ABO26102.1, YP_{—}004535707.1, ACJ22762.1, ABO26097.1, BAC65444.1, ABO61829.1, YP_{—}114083.1, CAH55828.1, ABO26106.1, YP_{—}552229.1, NP_{—}049190.1, ABO26116.1, CAH56107.1, CAM32407.1, ABO26101.1, ABO61841.1, ABM79805.1, ZP_{—}05075249.1, AAC27438.2, YP_{—}003754872.1, ADC29532.1, ADA71139.1, ADA71107.1, ADA71095.1, YP_{—}001268217.1, ADA71126.1, ADA71094.1, CAH56108.1, ADC29533.1, ADA71085.1, ZP_{—}05054453.1, ADA71097.1, ADA71086.1, ADA71114.1, ADC29548.1, ADA71101.1, ADC29547.1, ADA71138.1, ADC29542.1, ADA71098.1, ADA71128.1, ADA71105.1, ADA71093.1, ADA71135.1, ADA71100.1, YP_{—}557479.1, ADA71113.1, ADA71091.1, ADC29537.1, ADA71084.1, ADA71090.1, CAH56094.1, XP_{—}002945767.1, ADA71137.1, ADA71103.1, ADA71118.1, ADA71133.1, ADA71102.1, ADC29536.1, CAH56100.1, CAH56101.1, ACI15225.1, ACI15225.1, ABO26091.1, CAH55826.1, CAH55824.1, ZP_{—}08484419.1, ADA71111.1, ACJ22759.1, CAH55825.1, CAH56106.1, CAH56099.1, CAC40957.1, ZP_{—}05075037.1, CAH56102.1, ZP_{—}06846296.1, ABJ16491.1, ZP_{—}05067177.1, XP_{—}001698107.1, BAH10789.1, BAH10791.1, BAH10793.1, BAH10788.1, ABJ16490.1, BAH10800.1, BAH10790.1, BAH10792.1, ZP_{—}05075214.1, BAH10799.1, BAH10795.1, BAH10787.1, BAH10798.1, BAH10794.1, BAH10801.1, BAH10796.1, BAH10797.1, BAH10802.1, CAH56095.1, CAH56096.1, ADC29538.1, ABX76425.1, ZP_{—}06727686.1, ZP_{—}07774883.1, YP_{—}001615042.1, in particular YP_{—}001185946.1, Q9WWW6.1, YP_{—}957898.1, YP_{—}957728.1, YP_{—}694427.1, BAC98365.1, ZP_{—}00957064.1, CAC86944.1, YP_{—}001672212.1, CAB59525.1, ACH99213.1, ACH99215.1, ACH99216.1, AAK56792.1, ACH99229.1, ACS91348.1, AAP41820.1 and particularly preferably YP_{—}001185946.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues are modified compared to the aforementioned reference sequences by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E_{1b} in general is understood to mean in particular the conversion of lauric acid and/or methyl laurate to ω-hydroxylauric acid and/or methyl ω-hydroxylaurate.

### Auxiliary Enzymes to E₁

It is preferable according to the invention that, if E₁ₐ is a eukaryotic P450 alkane hydroxylase, the microorganism according to the invention also has increased activity of a NADPH cytochrome P450 oxidoreductase of EC 1.6.2.4 in comparison to its wild type. This has the technical effect that the activity of the eukaryotic P450 alkane hydroxylases is increased and the product yields increased.

NADPH cytochrome P450 oxidoreductases of EC 1.6.2.4 catalyse the following reaction: oxidized cytochrome P450+NADPH⁺=reduced cytochrome P450+NADP⁺+H⁺

It is preferable according to the invention that, if E₁ₐ is a prokaryotic P450 alkane hydroxylase of the CYP_{—}153 type, the microorganism according to the invention also has increased activity of a ferredoxin NAD(P)⁺ reductase of EC 1.18.1.2 or EC 1.18.1.3 and/or of a ferredoxin, in comparison to its wild type. This has the technical effect that the activity of the prokaryotic P450 alkane hydroxylase of the CYP_{—}153 type is increased and the product yields increased. Ferredoxin NAD(P)⁺ reductases of EC 1.18.1.2 or EC 1.18.1.3 catalyse the following reaction: oxidized ferredoxin+NAD(P)H+ H⁺=reduced ferredoxin+NAD(P)⁺ and are preferably encoded by a gene which is situated in direct proximity to a gene of an aforementioned prokaryotic P450 alkane hydroxylase of the CYP_{—}153 type or of a ferredoxin described in connection with this invention.

The term “in direct proximity” means that a maximum of three other structural genes are located between the genes in question.

Ferredoxins catalyse the following reactions:

alkane hydroxylase+reduced ferredoxin+alkanoic acid (ester)=alkane monooxygenase+oxidized ferredoxin+ω-hydroxyalkanoic acid (ester)+H₂O,
alkane hydroxylase+2 reduced ferredoxins+alkanoic acid (ester)=alkane hydroxylase+2 oxidized ferredoxins+ω-oxoalkanoic acid (ester)+2H₂O or
alkane hydroxylase+3 reduced ferredoxins+alkanoic acid (ester)=alkane hydroxylase+3 oxidized ferredoxins+ω-carboxyalkanoic acid (ester)+3H₂O and
are preferably encoded by a gene which is situated in direct proximity to a gene of an aforementioned prokaryotic P450 alkane hydroxylase of the CYP_{—}153 type or an aforementioned ferredoxin NAD(P)⁺ reductase of EC 1.18.1.2 or EC 1.18.1.3. The term “in direct proximity” means that a maximum of three other structural genes are located between the genes in question.

Preferred microorganisms display increased activity of the ferredoxin NAD(P)⁺ reductase AlkT and of a ferredoxin in comparison to its wild type.

It is preferable according to the invention that, if E₁ is an AlkB alkane hydroxylase of

EC 1.14.15.3, the microorganism according to the invention also displays increased activity of an AlkT rubredoxin NAD(P)⁺ reductase of EC 1.18.1.1 or of EC 1.18.1.4 and/or of a rubredoxin AlkG in comparison to its wild type. This has the technical effect that the activity of the AlkB alkane hydroxylase is raised and the product yields increased.
AlkT rubredoxin NAD(P)⁺ reductases of EC 1.18.1.1 or EC 1.18.1.4 catalyse the following reaction:
oxidized rubredoxin+NAD(P)H+H⁺=reduced rubredoxin+NAD(P)⁺ and are preferably encoded by a gene which is situated in direct proximity to a gene of an aforementioned AlkB alkane hydroxylase of EC 1.14.15.3 or of a rubredoxin AlkG described in connection with this invention.

The term “in direct proximity” means that a maximum of three other structural genes are located between the genes in question.

Rubredoxins AlkG catalyse the following reactions:

alkane monooxygenase+reduced rubredoxin+alkanoic acid (ester)=alkane monooxygenase+oxidized rubredoxin+ω-hydroxyalkanoic acid (ester)+H₂O, alkane monooxygenase+2 reduced rubredoxins+alkanoic acid (ester)=alkane monooxygenase+2 oxidized rubredoxins+ω-oxoalkanoic acid (ester)+2H₂O or
alkane monooxygenase+3 reduced rubredoxins+alkanoic acid (ester)=alkane monooxygenase+3 oxidized rubredoxins+ω-carboxyalkanoic acid (ester)+3H₂O and
are preferably encoded by a gene which is situated in direct proximity to a gene of an aforementioned AlkB alkane hydroxylase of EC 1.14.15.3 or an aforementioned AlkT rubredoxin NAD(P)⁺ reductase of EC 1.18.1.1 or EC 1.18.1.4. The term “in direct proximity” means that a maximum of three other structural genes are located between the genes in question.

Preferred microorganisms display increased activity of the AlkT rubredoxin NAD(P)⁺ reductase and of the rubredoxin AlkG in comparison to its wild type.

### Enzyme E₂ ω-Amination

It is preferable according to the invention to provide a microorganism which in particular is capable of producing ω-functionalized carboxylic acids and ω-functionalized carboxylate esters from at least one simple carbon source, where the ω-functionalization corresponds to an amino group, in particular primary, in the ω position. Thereby, the microorganisms can advantageously be used in processes for the production of ω-aminocarboxylic acids or ω-aminocarboxylate esters.

Hence microorganisms preferred according to the invention are characterized in that the second genetic modification additionally comprises that the microorganism displays increased activity of an enzyme E₂, which catalyses the conversion of ω-oxocarboxylic acids or ω-oxocarboxylate esters to the corresponding ω-aminocarboxylic acids or ω-aminocarboxylate esters, in comparison to its wild type.

According to the invention, the enzyme E₂ is preferably an ω-transaminase of EC 2.6.1.-.

As a measure of the enzyme activity E₂, the conversion of ω-oxolauric acid and/or methyl ω-oxolaurate to ω-aminolauric acid and/or methyl ω-aminolaurate can in particular be utilized.

Preferred enzymes E₂ are selected from the group:

3HMU_A, AAD41041.1, AAK15486.1, ABE03917.1, ADR60699.1, ADR61066.1, ADR62525.1, AEL07495.1, CAZ86955.1, EFW82310.1, EFW87681.1, EGC99983.1, EGD03176.1, EGE58369.1, EGH06681.1, EGH08331.1, EGH24301.1, EGH32343.1, EGH46412.1, EGH55033.1, EGH62152.1, EGH67339.1, EGH70821.1, EGH71404.1, EGH78772.1, EGH85312.1, EGH97105.1, EGP57596.1, NP_{—}102850.1, NP_{—}106560.1, NP_{—}248912.1, NP_{—}248990.1, NP_{—}354026.2, NP_{—}421926.1, NP_{—}637699.1, NP_{—}642792.1, NP_{—}744329.1, NP_{—}744732.1, NP_{—}747283.1, NP_{—}795039.1, NP_{—}901695.1 (encoded by SEQ ID No. 12), XP_{—}002943905.1, YP_{—}001021095.1, YP_{—}001059677.1, YP_{—}001061726.1, YP_{—}001066961.1, YP_{—}001074671.1, YP_{—}001120907.1, YP_{—}001140117.1, YP_{—}001170616.1, YP_{—}001185848.1, YP_{—}001188121.1, YP_{—}001233688.1, YP_{—}001268866.1, YP_{—}001270391.1, YP_{—}001345703.1, YP_{—}001412573.1, YP_{—}001417624.1, YP_{—}001526058.1, YP_{—}001579295.1, YP_{—}001581170.1, YP_{—}001668026.1, YP_{—}001669478.1, YP_{—}001671460.1, YP_{—}001685569.1, YP_{—}001747156.1, YP_{—}001749732.1, YP_{—}001765463.1, YP_{—}001766294.1, YP_{—}001790770.1, YP_{—}001808775.1, YP_{—}001809596.1, YP_{—}001859758.1, YP_{—}001888405.1, YP_{—}001903233.1, YP_{—}001977571.1, YP_{—}002229759.1, YP_{—}002231363.1, YP_{—}002280472.1, YP_{—}002297678.1, YP_{—}002543874.1, YP_{—}002549011.1, YP_{—}002796201.1, YP_{—}002801960.1, YP_{—}002875335.1, YP_{—}002897523.1, YP_{—}002912290.1, YP_{—}002974935.1, YP_{—}003060891.1, YP_{—}003264235.1, YP_{—}003552364.1, YP_{—}003578319.1, YP_{—}003591946.1, YP_{—}003607814.1, YP_{—}003641922.1, YP_{—}003674025.1, YP_{—}003692877.1, YP_{—}003755112.1, YP_{—}003896973.1, YP_{—}003907026.1, YP_{—}003912421.1, YP_{—}004086766.1, YP_{—}004142571.1, YP_{—}004147141.1, YP_{—}004228105.1, YP_{—}004278247.1, YP_{—}004305252.1, YP_{—}004356916.1, YP_{—}004361407.1, YP_{—}004378186.1, YP_{—}004379856.1, YP_{—}004390782.1, YP_{—}004472442.1, YP_{—}004590892.1, YP_{—}004612414.1, YP_{—}004676537.1, YP_{—}004693233.1, YP_{—}004701580.1, YP_{—}004701637.1, YP_{—}004704442.1, YP_{—}108931.1, YP_{—}110490.1, YP_{—}168667.1, YP_{—}237931.1, YP_{—}260624.1, YP_{—}262985.1, YP_{—}271307.1, YP_{—}276987.1, YP_{—}334171.1, YP_{—}337172.1, YP_{—}350660.1, YP_{—}351134.1, YP_{—}364386.1, YP_{—}366340.1, YP_{—}369710.1, YP_{—}370582.1, YP_{—}426342.1, YP_{—}440141.1, YP_{—}442361.1, YP_{—}468848.1, YP_{—}521636.1, YP_{—}554363.1, YP_{—}608454.1, YP_{—}610700.1, YP_{—}614980.1, YP_{—}622254.1, YP_{—}625753.1, YP_{—}680590.1, YP_{—}751687.1, YP_{—}767071.1, YP_{—}774090.1, YP_{—}774932.1, YP_{—}788372.1, YP_{—}858562.1, YP_{—}928515.1, YP_{—}983084.1, YP_{—}995622.1, ZP_{—}00948889.1, ZP_{—}00954344.1, ZP_{—}00959736.1, ZP_{—}00998881.1, ZP_{—}01011725.1, ZP_{—}01037109.1, ZP_{—}01058030.1, ZP_{—}01076707.1, ZP_{—}01103959.1, ZP_{—}01167926.1, ZP_{—}01224713.1, ZP_{—}01442907.1, ZP_{—}01446892.1, ZP_{—}01550953.1, ZP_{—}01625518.1, ZP_{—}01745731.1, ZP_{—}01750280.1, ZP_{—}01754305.1, ZP_{—}01763880.1, ZP_{—}01769626.1, ZP_{—}01865961.1, ZP_{—}01881393.1, ZP_{—}01901558.1, ZP_{—}02145337.1, ZP_{—}02151268.1, ZP_{—}02152332.1, ZP_{—}02167267.1, ZP_{—}02190082.1, ZP_{—}02242934.1, ZP_{—}02360937.1, ZP_{—}02367056.1, ZP_{—}02385477.1, ZP_{—}02456487.1, ZP_{—}02883670.1, ZP_{—}03263915.1, ZP_{—}03263990.1, ZP_{—}03400081.1, ZP_{—}03452573.1, ZP_{—}03456092.1, ZP_{—}03517291.1, ZP_{—}03529055.1, ZP_{—}03571515.1, ZP_{—}03572809.1, ZP_{—}03587785.1, ZP_{—}03588560.1, ZP_{—}03697266.1, ZP_{—}03697962.1, ZP_{—}04521092.1, ZP_{—}04590693.1, ZP_{—}04890914.1, ZP_{—}04891982.1, ZP_{—}04893793.1, ZP_{—}04902131.1, ZP_{—}04905327.1, ZP_{—}04941068.1, ZP_{—}04944536.1, ZP_{—}04945255.1, ZP_{—}04959332.1, ZP_{—}04964181.1, ZP_{—}05053721.1, ZP_{—}05063588.1, ZP_{—}05073059.1, ZP_{—}05077806.1, ZP_{—}05082750.1, ZP_{—}05091128.1, ZP_{—}05095488.1, ZP_{—}05101701.1, ZP_{—}05116783.1, ZP_{—}05121836.1, ZP_{—}05127756.1, ZP_{—}05637806.1, ZP_{—}05742087.1, ZP_{—}05783548.1, ZP_{—}05786246.1, ZP_{—}05843149.1, ZP_{—}05945960.1, ZP_{—}06459045.1, ZP_{—}06487195.1, ZP_{—}06492453.1, ZP_{—}06493162.1, ZP_{—}06703644.1, ZP_{—}06731146.1, ZP_{—}06839371.1, ZP_{—}07007312.1, ZP_{—}07266194.1, ZP_{—}07374050.1, ZP_{—}07662787.1, ZP_{—}07778196.1, ZP_{—}07797983.1, ZP_{—}08099459.1, ZP_{—}08138203.1, ZP_{—}08141719.1, ZP_{—}08142973.1, ZP_{—}08177102.1, ZP_{—}08185821.1, ZP_{—}08186468.1, ZP_{—}08208888.1, ZP_{—}08266590.1, ZP_{—}08402041.1, ZP_{—}08406891.1, ZP_{—}08522175.1, ZP_{—}08527488.1, ZP_{—}08631252.1, ZP_{—}08636687.1, SEQ ID No. 08, SEQ ID No. 09
in particular NP_{—}901695.1 (encoded by SEQ ID No. 12), ZP_{—}03697266.1, AAD41041.1, YP_{—}002796201.1, ZP_{—}03697962.1, YP_{—}001859758.1, YP_{—}002229759.1, YP_{—}001120907.1, YP_{—}110490.1, ZP_{—}04964181.1, YP_{—}774932.1, YP_{—}001766294.1, YP_{—}001581170.1, YP_{—}622254.1, ZP_{—}03588560.1, YP_{—}001809596.1, YP_{—}370582.1, ZP_{—}03572809.1, NP_{—}248990.1, YP_{—}001888405.1, ZP_{—}04905327.1, YP_{—}001061726.1, YP_{—}001668026.1, ZP_{—}01750280.1, ZP_{—}07778196.1, EGH71404.1, NP_{—}744329.1, YP_{—}004147141.1, ADR61066.1, ZP_{—}05783548.1, YP_{—}004701637.1, YP_{—}366340.1, YP_{—}003264235.1, EGD03176.1, YP_{—}001268866.1, ZP_{—}01901558.1, ZP_{—}05121836.1, YP_{—}003692877.1, ZP_{—}03517291.1, YP_{—}002974935.1, YP_{—}001668026.1, ADR61066.1, NP_{—}744329.1, YP_{—}001268866.1, YP_{—}004701637.1, ZP_{—}08142973.1, ADR62525.1, YP_{—}610700.1, NP_{—}747283.1, ADR62525.1, YP_{—}001270391.1, YP_{—}004704442.1, YP_{—}610700.1, YP_{—}001747156.1, ZP_{—}08138203.1, ZP_{—}07266194.1, EGH70821.1, YP_{—}351134.1, EGH32343.1, EGH08331.1, EGH67339.1, YP_{—}001668026.1, YP_{—}004701637.1, YP_{—}237931.1, ZP_{—}03400081.1, ZP_{—}05116783.1, ZP_{—}01550953.1, ZP_{—}07662787.1, YP_{—}928515.1, YP_{—}788372.1, YP_{—}001021095.1, ZP_{—}07797983.1, YP_{—}003578319.1, YP_{—}004305252.1, NP_{—}248912.1, ZP_{—}08636687.1, YP_{—}003912421.1, YP_{—}751687.1, ZP_{—}08142973.1, YP_{—}271307.1, ZP_{—}05082750.1, YP_{—}001417624.1, YP_{—}353455.1, SEQ ID No. 08, SEQ ID No. 09 and particularly preferably NP_{—}901695.1 (encoded by SEQ ID No. 12), YP_{—}353455.1, SEQ ID No. 08 and SEQ ID No. 09
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E₂ in general is understood to mean in particular the conversion of ω-oxolauric acid and/or methyl ω-oxolaurate to ω-aminolauric acid and/or methyl ω-aminolaurate.

A microorganism according to the invention with increased activity of an enzyme E₂ in comparison to its wild type advantageously displays in comparison to its wild type decreased activity of an aldehyde dehydrogenase of EC 1.2.1.3, EC 1.2.1.4 or EC 1.2.1.5, which catalyses the following reaction:

ω-oxoalkanoic acid (ester)+NAD(P)⁺=ω-carboxyalkanoic acid (ester)+NAD(P)H+ H⁺ Such aldehyde dehydrogenases are in particular those which are listed below as specific E₅, and those which are listed below as preferred E₄ fatty alcohol oxidases of EC 1.1.3.20, AlkJ alcohol dehydrogenases of EC 1.1.99.- and alcohol dehydrogenases of EC 1.1.1.1 or EC 1.1.1.2 and catalyse at least the second of the two reactions mentioned there; such enzymes are also described below as enzymes E_{4*}.

This has the technical effect that depletion of the ω-oxocarboxylic acid or the ω-oxocarboxylate ester to be aminated is prevented and hence more substrate is available for the ω-aminocarboxylic acid or ω-aminocarboxylate ester product formation.

The wording “in comparison to its wild type, decreased activity” is preferably understood to mean activity decreased by at least 50%, particularly preferably by at least 90%, more preferably by at least 99.9%, still more preferably by at least 99.99% and most preferably by at least 99.999% based on the wild type activity. The wording “decreased activity” also includes no detectable activity (“activity of nil”). The reduction of the activity of a specific enzyme can for example be effected by targetted mutation or by other measures known to those skilled in the art for reducing the activity of a specific enzyme. Further processes for reducing enzymatic activities in microorganisms are known to those skilled in the art. Molecular biological techniques in particular are suitable here. Those skilled in the art will find instructions for the modification and reduction of protein expression and enzymatic activity reduction associated therewith for Candida, in particular for interruption of specific genes in WO91/006660 and WO03/100013.

Microorganisms preferred according to the invention are characterized in that the reduction of the enzymatic activity is achieved by modification of a gene comprising a nucleic acid sequence coding for the aforementioned enzymes, wherein the modification is selected from the group comprising, preferably consisting of, insertion of foreign DNA into the gene, deletion at least of parts of the gene, point mutations in the gene sequence, RNA interference (siRNA), antisense RNA or modification (insertion, deletion or point mutations) of regulatory sequences which flank the gene. In this connection, foreign DNA should be understood to mean any DNA sequence which is “foreign” to the gene (and not to the organism). In this connection, it is particularly preferable that the gene is interrupted by insertion of a selection marker gene, so that the foreign DNA is a selection marker gene, wherein the insertion was preferably effected by homologous recombination into the gene locus. In this connection, it can be advantageous if the selection marker gene is extended with further functionalities which in turn enable subsequent removal from the gene. This can for example be achieved by recombination systems foreign to the organism, such as for example a Cre/loxP system or FRT (Flippase Recognition Target) system or the recombination system intrinsic to the organism.

The reduction of the activity of the microorganism according to the invention in comparison to its wild type is determined according to methods described above for the determination of the activity with use of as far as possible equal cell counts/concentrations, wherein the cells have been grown under the same conditions such as for example medium, gassing and stirring.

### Enzyme E₃ Cofactor Recycling ω-Amination

For the production of an ω-amino-functionalized carboxylic acid or an ω-amino-functionalized carboxylate ester it can be advantageous if the second genetic modification comprises increased activity of an enzyme E₃ which catalyses the conversion of an α-ketocarboxylic acid to an amino acid. Preferably the enzyme E₃ is an amino acid dehydrogenase, such as for example serine dehydrogenases, aspartate dehydrogenases, phenylalanine dehydrogenases and glutamate dehydrogenases, particularly preferably an alanine dehydrogenase of EC 1.4.1.1.

Such preferable alanine dehydrogenases are selected from

EGR93259.1, YP_{—}004743277.1, YP_{—}004741620.1, YP_{—}004737294.1, YP_{—}002509853.1, YP_{—}002492255.1, YP_{—}002489845.1, YP_{—}002481919.1, YP_{—}001819330.1, YP_{—}004728333.1, ZP_{—}08670930.1, YP_{—}004672392.1, YP_{—}004467026.1, YP_{—}004326214.1, YP_{—}002349951.1, YP_{—}001674437.1, YP_{—}003921585.1, YP_{—}001699731.1, YP_{—}004720756.1, YP_{—}004719515.1, EGQ22316.1, EGQ21760.1, YP_{—}004689232.1, YP_{—}004698526.1, YP_{—}004694875.1, EGP67576.1, YP_{—}001832691.1, YP_{—}001760857.1, AEJ53875.1, AEJ42949.1, YP_{—}004392931.1, YP_{—}004404798.1, YP_{—}004374160.1, YP_{—}004303162.1, YP_{—}004196134.1, YP_{—}004178581.1, YP_{—}004163857.1, YP_{—}004161555.1, YP_{—}004099081.1, YP_{—}004101986.1, YP_{—}004042336.1, YP_{—}003994181.1, YP_{—}003966543.1, YP_{—}003913256.1, YP_{—}003825828.1, YP_{—}003806106.1, YP_{—}003686355.1, YP_{—}003678575.1, YP_{—}003654745.1, YP_{—}003651439.1, YP_{—}003637111.1, YP_{—}003631815.1, YP_{—}003300711.1, YP_{—}002886396.1, ZP_{—}03493991.1, YP_{—}001890813.1, YP_{—}001888849.1, YP_{—}001554753.1, YP_{—}001529018.1, YP_{—}001528954.1, YP_{—}001502090.1, YP_{—}001412833.1, YP_{—}001363812.1, YP_{—}923679.1, NP_{—}440110.1, ZP_{—}08640273.1, ZP_{—}08639751.1, ZP_{—}08637916.1, YP_{—}004171395.1, YP_{—}001366419.1, YP_{—}001327051.1, YP_{—}001262560.1, YP_{—}886996.1, YP_{—}882850.1, YP_{—}704410.1, YP_{—}703508.1, ZP_{—}08624689.1, YP_{—}001230376.1, P17557.1, P17556.1, CCB94892.1, CCB73698.1, YP_{—}001168635.1, YP_{—}004668736.1, YP_{—}911378.1, YP_{—}003686997.1, YP_{—}002263235.1, NP_{—}820115.1, YP_{—}004653761.1, YP_{—}004651159.1, YP_{—}003869397.1, YP_{—}004641708.1, YP_{—}004641134.1, YP_{—}001996597.1, YP_{—}001998297.1, YP_{—}001943676.1, YP_{—}001810799.1, YP_{—}004630087.1, YP_{—}004621893.1, YP_{—}004613083.1, ZP_{—}08621144.1, YP_{—}003954200.1, YP_{—}001372688.1, YP_{—}001233686.1, ZP_{—}08594848.1, ZP_{—}08586665.1, ZP_{—}08578896.1, ZP_{—}08575937.1, YP_{—}004604438.1, YP_{—}004600931.1, ZP_{—}08569139.1, ZP_{—}08566255.1, AEB25326.1, YP_{—}374584.1, YP_{—}004216732.1, ZP_{—}06806151.1, ZP_{—}06440291.1, ZP_{—}06369993.1, ZP_{—}06254238.1, ZP_{—}05844252.1, ZP_{—}05472927.1, ZP_{—}05365401.1, ZP_{—}04747945.1, ZP_{—}04678933.1, ZP_{—}03779761.1, ZP_{—}03728859.1, ZP_{—}03711891.1, ZP_{—}03697269.1, ZP_{—}01628294.1, ZP_{—}01546224.1, ZP_{—}01444021.1, ZP_{—}01308570.1, ZP_{—}01228194.1, ZP_{—}01164841.1, ZP_{—}01114638.1, YP_{—}004566582.1, YP_{—}004572166.1, YP_{—}004571401.1, YP_{—}004569425.1, YP_{—}003513168.1, YP_{—}004561169.1, ZP_{—}08554945.1, YP_{—}400777.1, ZP_{—}08533479.1, ZP_{—}08533412.1, ZP_{—}08525779.1, ZP_{—}08523693.1, YP_{—}004471329.1, YP_{—}004368103.1, YP_{—}001536790.1, YP_{—}001158763.1, YP_{—}662032.1, YP_{—}967824.1, YP_{—}004542206.1, YP_{—}002958019.1, YP_{—}645630.1, ZP_{—}08520595.1, AEG81976.1, YP_{—}002560779.1, YP_{—}496956.1, YP_{—}411850.1, YP_{—}300065.1, NP_{—}840123.1, ZP_{—}08514775.1, YP_{—}002250769.1, YP_{—}002155665.1, YP_{—}002137991.1, YP_{—}001135275.1, YP_{—}001070365.1, YP_{—}639268.1, NP_{—}864377.1, YP_{—}004554709.1, YP_{—}004546384.1, YP_{—}004544159.1, ZP_{—}01448725.1, ZP_{—}01255407.1, EGL88594.1, EGL87587.1, YP_{—}004536059.1, ZP_{—}08512666.1, ZP_{—}08501410.1, ZP_{—}08493566.1, ZP_{—}08486369.1, YP_{—}004497891.1, YP_{—}004494473.1, YP_{—}003945301.1, YP_{—}003835539.1, YP_{—}003634898.1, YP_{—}003503876.1, ZP_{—}06503131.1, YP_{—}003376450.1, YP_{—}003409976.1, YP_{—}003409004.1, YP_{—}003395275.1, YP_{—}003393138.1, YP_{—}003387714.1, YP_{—}003382934.1, ZP_{—}05760008.1, ZP_{—}05300490.1, ZP_{—}04387987.1, ZP_{—}03725713.1, YP_{—}002134125.1, YP_{—}001618802.1, ZP_{—}01899015.1, ZP_{—}01881250.1, ZP_{—}01731833.1, YP_{—}004529602.1, YP_{—}004512974.1, YP_{—}004479110.1, YP_{—}004434722.1, YP_{—}004430602.1, CBX28458.1, ZP_{—}05217624.1, ZP_{—}01074124.1, ZP_{—}01062209.1, ZP_{—}01011939.1, ZP_{—}00956754.1, YP_{—}388045.1, ZP_{—}07910902.1, ZP_{—}07835291.1, ZP_{—}07831081.1, ZP_{—}07704117.1, ZP_{—}07112933.1, ZP_{—}06860168.1, ZP_{—}05915689.1, YP_{—}002352943.1, YP_{—}826544.1, YP_{—}004087624.1, ADP99134.1, YP_{—}003590847.1, YP_{—}003589189.1, YP_{—}001192379.1, ZP_{—}08473868.1, ZP_{—}08469833.1, ZP_{—}08462614.1, ZP_{—}07709417.1, ZP_{—}07672507.1, ZP_{—}07608107.1, ZP_{—}07404685.1, ZP_{—}07334010.1, ZP_{—}07333254.1, ZP_{—}06888732.1, ZP_{—}06837313.1, YP_{—}873046.1, YP_{—}004060177.1, YP_{—}004007860.1, YP_{—}003492711.1, ZP_{—}08456143.1, YP_{—}003675989.1, YP_{—}003159562.1, NP_{—}302068.1, YP_{—}004461013.1, ZP_{—}08426378.1, ZP_{—}08422563.1, YP_{—}004122643.1, YP_{—}004077807.1, YP_{—}004058618.1, YP_{—}004055696.1, YP_{—}003898888.1, YP_{—}003575339.1, ZP_{—}06186049.1, YP_{—}003314861.1, YP_{—}003148148.1, YP_{—}002786543.1, YP_{—}001661762.1, YP_{—}001666058.1, YP_{—}001549204.1, YP_{—}001518627.1, YP_{—}004453289.1, YP_{—}004450492.1, YP_{—}004301609.1, YP_{—}465316.1, ZP_{—}08411512.1, YP_{—}001394062.1, YP_{—}001035553.1, YP_{—}417038.1, YP_{—}301147.1, YP_{—}014199.1, EGJ45059.1, EGJ36821.1, EGJ36552.1, EGJ19019.1, ZP_{—}08388916.1, YP_{—}004427278.1, YP_{—}003909234.1, YP_{—}002536659.1, YP_{—}001940410.1, YP_{—}001329977.1, YP_{—}001323343.1, YP_{—}001114195.1, YP_{—}001096594.1, YP_{—}949547.1, YP_{—}756289.1, YP_{—}722774.1, YP_{—}525283.1, YP_{—}461225.1, YP_{—}320697.1, YP_{—}289022.1, YP_{—}075651.1, NP_{—}988633.1, YP_{—}004399762.1, YP_{—}004335185.1, ADX76365.1, YP_{—}004203407.1, YP_{—}001917832.1, YP_{—}001642542.1, ZP_{—}08332142.1, YP_{—}041174.1, ZP_{—}08328264.1, YP_{—}004225082.1, EGG96712.1, ZP_{—}08311476.1, ZP_{—}08310170.1, ZP_{—}08267322.1, ZP_{—}08263846.1, ZP_{—}07898723.1, YP_{—}003273311.1, ZP_{—}05909597.1, YP_{—}003073095.1, YP_{—}003022905.1, YP_{—}003013384.1, YP_{—}003011072.1, ZP_{—}04777180.1, ZP_{—}04432601.1, YP_{—}001016505.1, YP_{—}953175.1, YP_{—}731492.1, ZP_{—}08302086.1, ZP_{—}08296718.1, ZP_{—}08285373.1, ZP_{—}08280138.1, ZP_{—}08270040.1, ZP_{—}08261780.1, ZP_{—}08258406.1, ZP_{—}08246570.1, YP_{—}003113209.1, YP_{—}002436565.1, ZP_{—}04409790.1, YP_{—}428767.1, EGG40837.1, CCA54694.1, YP_{—}004147180.1, YP_{—}550034.1, YP_{—}173042.1, EGF75662.1, YP_{—}004205024.1, YP_{—}003670363.1, YP_{—}003476027.1, YP_{—}003241464.1, YP_{—}863990.1, YP_{—}004149630.1, YP_{—}003646700.1, EGF24326.1, BAK15593.1, YP_{—}003991014.1, YP_{—}003988127.1, YP_{—}003722297.1, YP_{—}003254539.1, YP_{—}003251916.1, NP_{—}901692.1, EGF16043.1, EGF07290.1, YP_{—}003048854.1, YP_{—}149301.1, YP_{—}148605.1, YP_{—}004340432.1, EFT09946.1, EFS80513.1, EFS51332.1, EFS42459.1, YP_{—}003060895.1, YP_{—}003059033.1, ZP_{—}03305373.1, YP_{—}002379520.1, YP_{—}372555.1, NP_{—}085655.1, YP_{—}004321492.1, ZP_{—}08239446.1, YP_{—}003817108.1, YP_{—}002951286.1, YP_{—}002950656.1, YP_{—}002522266.1, YP_{—}001982538.1, YP_{—}001127463.1, YP_{—}001126767.1, NP_{—}764939.1, NP_{—}761756.1, NP_{—}244046.1, NP_{—}243195.1, YP_{—}003194671.1, YP_{—}003161559.1, YP_{—}002797803.1, YP_{—}002634404.1, YP_{—}439119.1, YP_{—}314402.1, YP_{—}143482.1, NP_{—}295618.1, ZP_{—}08215173.1, YP_{—}004282846.1, YP_{—}004267961.1, YP_{—}001867313.1, YP_{—}001301882.1, YP_{—}847214.1, YP_{—}004095847.1, YP_{—}003338282.1, YP_{—}003337256.1, YP_{—}355846.1, YP_{—}253131.1, ZP_{—}08197563.1, ZP_{—}08196283.1, ADW06447.1, YP_{—}003370508.1, YP_{—}003317645.1, YP_{—}003184411.1, YP_{—}003198349.1, YP_{—}003084639.1, YP_{—}004294565.1, YP_{—}004243057.1, CBZ55377.1, EGC26795.1, EGC25718.1, EGC23378.1, ZP_{—}07887872.1, YP_{—}003269716.1, YP_{—}003203632.1, YP_{—}003199972.1, YP_{—}003153148.1, YP_{—}003146304.1, YP_{—}002893498.1, ZP_{—}03230841.1, ZP_{—}03229411.1, YP_{—}001050520.1, YP_{—}963387.1, YP_{—}927645.1, YP_{—}869684.1, YP_{—}734091.1, NP_{—}372233.1, NP_{—}102173.1, ZP_{—}08170259.1, EGD36706.1, EGD32748.1, ZP_{—}08155540.1, YP_{—}004142849.1, YP_{—}002417649.1, YP_{—}001301040.1, YP_{—}001211208.1, YP_{—}266230.1, ZP_{—}08145165.1, YP_{—}001801454.1, YP_{—}001736003.1, YP_{—}833487.1, YP_{—}831236.1, YP_{—}384064.1, YP_{—}094958.1, YP_{—}009793.1, NP_{—}975075.1, NP_{—}847074.1, EGC82166.1, YP_{—}004261609.1, YP_{—}004255502.1, YP_{—}678603.1, YP_{—}004181700.1, ZP_{—}08122013.1, ADT87541.1, YP_{—}003524764.1, YP_{—}002992990.1, YP_{—}002992892.1, YP_{—}081348.1, YP_{—}080482.1, YP_{—}002476349.1, ZP_{—}08115025.1, ZP_{—}08114403.1, YP_{—}003552869.1, YP_{—}002358112.1, ZP_{—}08111138.1, YP_{—}003770046.1, YP_{—}003103898.1, ZP_{—}08101069.1, ZP_{—}08097706.1, ZP_{—}08094005.1, YP_{—}003167240.1, YP_{—}002371817.1, YP_{—}004231854.1, EGA98455.1, YP_{—}002430239.1, ZP_{—}01049900.1, NP_{—}769819.1, NP_{—}768378.1, YP_{—}001143837.1, YP_{—}001108475.1, YP_{—}906040.1, YP_{—}726477.1, YP_{—}575010.1, YP_{—}477594.1, YP_{—}474564.1, YP_{—}130399.1, YP_{—}129373.1, YP_{—}123314.1, NP_{—}810467.1, NP_{—}646469.1, NP_{—}626044.1, NP_{—}391071.1 (encoded by SEQ ID No. 11), ZP_{—}08086822.1, ZP_{—}08084776.1, ZP_{—}08083119.1, NP_{—}465104.1, NP_{—}374819.1, NP_{—}337355.1, NP_{—}217296.1, ZP_{—}08072064.1, YP_{—}004197762.1, ZP_{—}08065558.1, ZP_{—}08063535.1, ZP_{—}08061612.1, ZP_{—}08059482.1, ZP_{—}08057644.1, ZP_{—}08055701.1, ZP_{—}08049025.1, ZP_{—}08047015.1, ZP_{—}04062925.1, YP_{—}269473.1, ZP_{—}08033402.1, ZP_{—}07829339.1, ZP_{—}06603053.1, ZP_{—}08020768.1, ZP_{—}08013590.1, ZP_{—}08011832.1, YP_{—}003783744.1, YP_{—}002781576.1, YP_{—}002780533.1, ZP_{—}02195873.1, NP_{—}797482.1, ZP_{—}08006697.1, ZP_{—}08006365.1, ZP_{—}08005962.1, ZP_{—}08004522.1, EFV89241.1, ZP_{—}07980135.1, ZP_{—}07974222.1, ZP_{—}07970379.1, ZP_{—}07962751.1, ZP_{—}07953732.1, ZP_{—}07945354.1, ZP_{—}06273519.1, YP_{—}003428808.1, YP_{—}003426902.1, YP_{—}001711555.1, YP_{—}001703831.1, YP_{—}001621081.1, YP_{—}001223643.1, YP_{—}001228127.1, YP_{—}849789.1, YP_{—}759696.1, NP_{—}969291.1, NP_{—}896596.1, NP_{—}470950.1, YP_{—}359521.1, ZP_{—}01946735.1, ZP_{—}03631968.1, ZP_{—}01101833.1, YP_{—}002826017.1, YP_{—}003796926.1, ZP_{—}07873974.1, ZP_{—}07870908.1, ZP_{—}07645051.1, ZP_{—}07643260.1, ZP_{—}06611917.1, AAT40119.1, ZP_{—}07864946.1, YP_{—}004068409.1, YP_{—}002796203.1, YP_{—}002774420.1, YP_{—}003600348.1, YP_{—}003599946.1, YP_{—}003565624.1, YP_{—}003565223.1, YP_{—}335198.1, YP_{—}423850.1, YP_{—}155059.1, ZP_{—}07843538.1, ZP_{—}07841226.1, ZP_{—}03566837.1, EFS39373.1, EFS35044.1, ZP_{—}05287373.1, ZP_{—}05280407.1, ZP_{—}05224249.1, ZP_{—}04701236.1, ZP_{—}04692180.1, ZP_{—}03561728.1, ZP_{—}03227314.1, ZP_{—}02931419.1, ZP_{—}02731551.1, ZP_{—}02465413.1, ZP_{—}02451335.1, ZP_{—}02384332.1, ZP_{—}02381808.1, ZP_{—}02330643.1, YP_{—}004047600.1, EFR99988.1, EFR93766.1, EFR90643.1, EFR84459.1, ZP_{—}04059923.1, ZP_{—}03613601.1, ZP_{—}07743242.1, ZP_{—}07740118.1, ZP_{—}07728760.1, ZP_{—}07728640.1, YP_{—}003557047.1, ZP_{—}07204792.1, ZP_{—}07033145.1, ZP_{—}06949396.1, ZP_{—}06928932.1, ZP_{—}05692073.1, ZP_{—}05687006.1, ZP_{—}04867480.1, YP_{—}775531.1, CBE70214.1, ZP_{—}07721182.1, CBW22027.1, BAJ31519.1, ZP_{—}07694389.1, ZP_{—}07653390.1, ZP_{—}07548028.1, ZP_{—}07547185.1, ZP_{—}07462497.1, ZP_{—}07458778.1, ZP_{—}07399459.1, ZP_{—}07397253.1, ZP_{—}07397250.1, ZP_{—}07390390.1, ZP_{—}07390003.1, ZP_{—}07388675.1, ZP_{—}07367724.1, ZP_{—}07206561.1, ZP_{—}07053170.1, ZP_{—}07048770.1, ZP_{—}06873224.1, ZP_{—}06852862.1, ZP_{—}06427630.1, ZP_{—}06307332.1, ZP_{—}06252577.1, ZP_{—}06175164.1, ZP_{—}06080808.1, ZP_{—}06052314.1, ZP_{—}06033748.1, ZP_{—}05945907.1, ZP_{—}05924840.1, ZP_{—}05885109.1, ZP_{—}05882095.1, ZP_{—}05877865.1, ZP_{—}05855512.1, ZP_{—}05745159.1, ZP_{—}05716384.1, ZPO4866524.1, ZP_{—}04819572.1, ZP_{—}04797418.1, ZPO4319784.1, ZP_{—}04302850.1, ZP_{—}04298961.1, ZP_{—}04287684.1, ZP_{—}04277177.1, ZP_{—}04248389.1, ZP_{—}04235899.1, ZP_{—}04230016.1, ZP_{—}04226233.1, ZP_{—}04219330.1, ZP_{—}04216141.1, ZP_{—}04209092.1, ZP_{—}04188247.1, ZP_{—}04184510.1, ZP_{—}04176651.1, ZP_{—}04172877.1, ZP_{—}04170954.1, ZP_{—}04166021.1, ZP_{—}04160852.1, ZP_{—}04158983.1, ZP_{—}04154769.1, ZP_{—}04153266.1, ZP_{—}04149717.1, ZP_{—}04122524.1, ZP_{—}04110635.1, ZP_{—}04109769.1, ZP_{—}04109049.1, ZPO4108444.1, ZP_{—}04104350.1, ZP_{—}04100460.1, ZP_{—}04075249.1, ZP_{—}04074263.1, ZP_{—}04009917.1, ZP_{—}03916440.1, ZP_{—}03703407.1, ZP_{—}03675960.1, ZP_{—}03588177.1, ZP_{—}03569636.1, ZP_{—}03497916.1, ZP_{—}03459468.1, ZP_{—}03299979.1, ZP_{—}03127493.1, ZP_{—}03054334.1, ZP_{—}03015779.1, ZP_{—}02478038.1, ZP_{—}02434435.1, ZP_{—}01891777.1, ZP_{—}01134782.1, ZP_{—}01084087.1, ZP_{—}00959435.1, ZP_{—}06021901.1, ZP_{—}02908521.1, ZP_{—}02892318.1, ZP_{—}02883918.1, ZP_{—}02433787.1, ZP_{—}02428013.1, ZP_{—}02424229.1, ZP_{—}02420399.1, ZP_{—}02190089.1, ZP_{—}02184200.1, ZP_{—}02166566.1, ZP_{—}02159718.1, ZP_{—}02152178.1, ZP_{—}02147727.1, ZP_{—}02144676.1, ZP_{—}02078507.1, ZP_{—}02072824.1, ZP_{—}02067293.1, ZP_{—}02061844.1, ZP_{—}01996280.1, ZP_{—}01991915.1, ZP_{—}01958087.1, ZP_{—}01908911.1, ZP_{—}01901606.1, ZP_{—}01895406.1, ZP_{—}01872936.1, ZP_{—}01870578.1, ZP_{—}01863314.1, ZP_{—}01859623.1, ZP_{—}01852574.1, ZP_{—}01834861.1, ZP_{—}01816459.1, ZP_{—}01770050.1, ZP_{—}01754550.1, ZP_{—}01750331.1, ZP_{—}01746097.1, ZP_{—}01736276.1, ZP_{—}01723571.1, ZP_{—}01688551.1, ZP_{—}01666824.1, ZP_{—}01627178.1, ZP_{—}01623088.1, ZP_{—}01612926.1, ZP_{—}01470938.1, ZP_{—}01460341.1, ZP_{—}01452344.1, ZP_{—}01439206.1, ZP_{—}01386817.1, ZP_{—}01313561.1, ZP_{—}01304248.1, ZP_{—}01264036.1, ZP_{—}01261877.1, ZP_{—}01235013.1, ZP_{—}01233072.1, ZP_{—}01224625.1, ZP_{—}01223017.1, ZP_{—}01221216.1, ZP_{—}01215557.1, ZP_{—}01202668.1, ZP_{—}01159834.1, ZP_{—}01158968.1, ZP_{—}01157579.1, ZP_{—}01130649.1, ZP_{—}01126987.1, ZP_{—}01122900.1, ZP_{—}01118752.1, ZP_{—}01090470.1, ZP_{—}01067027.1, ZP_{—}01058751.1, ZP_{—}01043459.1, ZP_{—}01041526.1, ZP_{—}01036767.1, ZP_{—}01001935.1, ZP_{—}00995212.1, ZP_{—}00992904.1, ZP_{—}00962062.1, ZP_{—}00952239.1, ZP_{—}00741173.1, ZP_{—}00740055.1, ZP_{—}00738801.1, ZP_{—}00517716.1, ZP_{—}00231205.1, ZP_{—}00208007.1, YP_{—}003974610.1, YP_{—}003546595.1, YP_{—}002317127.1, ZP_{—}07313778.1, ZP_{—}07302778.1, ZP_{—}07298850.1, ZP_{—}07285992.1, ZP_{—}07282306.1, ZP_{—}07279420.1, ZP_{—}07270582.1, ZP_{—}07001670.1, YP_{—}003706150.1, ZP_{—}06916083.1, ZP_{—}06912607.1, ZP_{—}06707160.1, ZP_{—}06324727.1, ZP_{—}06199155.1, ZP_{—}06197322.1, ZP_{—}05788488.1, ZP_{—}05785587.1, ZP_{—}05779471.1, ZP_{—}05739072.1, ZP_{—}05649780.1, ZP_{—}05647025.1, ZP_{—}05546023.1, ZP_{—}05341228.1, ZP_{—}05256588.1, ZP_{—}05127284.1, ZP_{—}05121710.1, ZP_{—}05119732.1, ZP_{—}05105668.1, ZP_{—}05101668.1, ZP_{—}05095370.1, ZP_{—}05090860.1, ZP_{—}05080646.1, ZP_{—}05076859.1, ZP_{—}05069222.1, ZP_{—}05065142.1, ZP_{—}05056378.1, ZP_{—}05052029.1, ZP_{—}05046506.1, ZP_{—}05037402.1, ZP_{—}05033610.1, ZP_{—}05026858.1, ZP_{—}05001187.1, ZP_{—}04959306.1, ZP_{—}04947229.1, ZP_{—}04941878.1, ZP_{—}04896669.1, ZP_{—}04890139.1, ZP_{—}04852481.1, ZP_{—}04849996.1, ZP_{—}04608704.1, ZP_{—}04581931.1, ZP_{—}04555275.1, ZP_{—}04553607.1, ZP_{—}04545440.1, ZP_{—}04538537.1, YP_{—}002311919.1, ZP_{—}01052096.1, YP_{—}432286.1, ZP_{—}07039851.1, ZP_{—}07036831.1, ZP_{—}07035634.1, ZP_{—}06826623.1, ZP_{—}06202690.1, ZP_{—}06091438.1, ZP_{—}06060476.1, YP_{—}002955941.1, YP_{—}002764322.1, YP_{—}002761274.1, YP_{—}002754767.1, YP_{—}002605829.1, YP_{—}002544281.1, YP_{—}002453687.1, YP_{—}002444060.1, YP_{—}002369417.1, YP_{—}002365390.1, YP_{—}002297006.1, YP_{—}002233968.1, YP_{—}001861152.1, YP_{—}001850232.1, YP_{—}001827236.1, YP_{—}001815332.1, YP_{—}001661116.1, YP_{—}001647239.1, YP_{—}001643400.1, YP_{—}001625970.1, YP_{—}001584357.1, YP_{—}001488077.1, YP_{—}001473862.1, YP_{—}001450010.1, YP_{—}001444991.1, YP_{—}001424576.1, YP_{—}001422460.1, YP_{—}001376512.1, YP_{—}001373857.1, YP_{—}001217438.1, YP_{—}001155448.1, YP_{—}001117213.1, YP_{—}001094151.1, YP_{—}950353.1, YP_{—}949946.1, YP_{—}944887.1, YP_{—}854776.1, YP_{—}837848.1, YP_{—}795217.1, YP_{—}750481.1, YP_{—}746463.1, YP_{—}681383.1, YP_{—}673989.1, YP_{—}632321.1, YP_{—}624008.1, YP_{—}615612.1, YP_{—}611857.1, YP_{—}604242.1, YP_{—}562748.1, YP_{—}536656.1, YP_{—}517218.1, YP_{—}459264.1, YP_{—}382475.1, YP_{—}340233.1, YP_{—}295387.1, YP_{—}285355.1, YP_{—}204286.1, YP_{—}174267.1, YP_{—}165491.1, YP_{—}126314.1, YP_{—}111103.1, YP_{—}098760.1, YP_{—}082111.1, YP_{—}064280.1, YP_{—}064276.1, YP_{—}062161.1, YP_{—}056928.1, YP_{—}008485.1, YP_{—}005739.1, NP_{—}961822.1, NP_{—}953341.1, NP_{—}926915.1, NP_{—}875991.1, NP_{—}834329.1, NP_{—}830409.1, NP_{—}827683.1, NP_{—}694147.1, NP_{—}693109.1, NP_{—}682897.1, NP_{—}661601.1, NP_{—}621858.1, NP_{—}486395.1, NP_{—}385730.1, NP_{—}231539.1, ADL65712.1, XP_{—}003087064.1, YP_{—}003886520.1, YP_{—}003699559.1, YP_{—}003516134.1, ADI98200.1, BAI86717.1, YP_{—}003794343.1, YP_{—}003790454.1, ADI11356.1, YP_{—}003845821.1, ADK69870.1, YP_{—}003784546.1, CBW36497.1, CBW26165.1, YP_{—}003709979.1, CAQ50186.1, ZP_{—}06770463.1, CBK69442.1, YP_{—}003413835.1, YP_{—}003595089.1, ZP_{—}06807811.1, YP_{—}003582455.1, YP_{—}003464731.1, YP_{—}003496397.1, YP_{—}003421918.1, CBL07274.1, CBK64956.1, YP_{—}003508515.1, AAL87460.1, AAC23579.1, AAC23578.1, AAC23577.1, ACU78652.1, YP_{—}003471439.1, YP_{—}003452777.1, ZP_{—}06384971.1, ACY25368.1, ABC26869.1, AAP44334.1, EEZ80018.1, ZP_{—}05110458.1, 1PJB_A, ZP_{—}04717201.1, ZP_{—}04689103.1, ZP_{—}04658071.1, XP_{—}002364705.1, ACN89388.1, 2VHW_A, 2VHV_A, XP_{—}001324625.1, ABZ06259.1, ABR57171.1, CAO90307.1, CAM75354.1, CAA44791.1, BAA77513.1, EGR96638.1, EGR94699.1, ZP_{—}08693646.1, YP_{—}004740306.1, YP_{—}004738947.1, AEE73472.1, YP_{—}002478771.1, YP_{—}002018970.1, YP_{—}001953230.1, ZP_{—}08683223.1, YP_{—}004073823.1, EGQ99856.1, ZP_{—}08664912.1, EGQ79321.1, YP_{—}001681700.1, AEJ51356.1, YP_{—}004378292.1, YP_{—}004237802.1, YP_{—}004166920.1, YP_{—}004043011.1, YP_{—}003997728.1, YP_{—}002975437.1, YP_{—}002514072.1, YP_{—}001433829.1, YP_{—}001185975.1, YP_{—}004676549.1, YP_{—}004016358.1, YP_{—}911347.1, YP_{—}004658403.1, YP_{—}002015455.1, YP_{—}001996171.1, YP_{—}001998271.1, YP_{—}001960099.1, YP_{—}001942826.1, YP_{—}001130666.1, YP_{—}004608353.1, YP_{—}508400.1, YP_{—}374553.1, ZP_{—}06298411.1, ZP_{—}06044299.1, ZP_{—}04390473.1, ZP_{—}04055222.1, ZP_{—}03779980.1, ZP_{—}03729400.1, ZP_{—}03390832.1, YP_{—}004580682.1, YP_{—}001988281.1, YP_{—}644219.1, YP_{—}665459.1, NP_{—}895289.1, YP_{—}004275231.1, NP_{—}208189.1, BAJ60529.1, BAJ59008.1, BAJ57509.1, BAJ56032.1, ZP_{—}01254396.1, YP_{—}445036.1, EGL90046.1, YP_{—}004510847.1, ZP_{—}08450330.1, YP_{—}003387804.1, YP_{—}003058152.1, ZP_{—}03438664.1, ZP_{—}01884341.1, AEG33860.1, YP_{—}004429375.1, ZP_{—}08459444.1, ZP_{—}07909193.1, ZP_{—}07908670.1, EFT26139.1, EFT23947.1, EFT12708.1, EFT03750.1, EFS82814.1, EFS74272.1, EFS67128.1, ZP_{—}06844564.1, YP_{—}826658.1, YP_{—}001195249.1, YP_{—}003095978.1, YP_{—}469292.1, YP_{—}004442054.1, YP_{—}004461174.1, YP_{—}004055616.1, YP_{—}003576656.1, YP_{—}003094537.1, YP_{—}001295973.1, AEE71143.1, YP_{—}004447480.1, YP_{—}001978005.1, ZP_{—}08413507.1, ZP_{—}07820264.1, YP_{—}416780.1, EGI86036.1, YP_{—}003109321.1, YP_{—}001275268.1, YP_{—}380171.1, YP_{—}159073.1, YP_{—}004203456.1, YP_{—}003761844.1, YP_{—}040853.1, ZP_{—}08328557.1, CBL87253.1, CBL87167.1, YP_{—}004316768.1, EFS92548.1, YP_{—}001016505.1, EGG67688.1, YP_{—}003528837.1, YP_{—}002434942.1, YP_{—}117835.1, YP_{—}004150583.1, YP_{—}003755105.1, YP_{—}002526442.1, YP_{—}003120958.1, EGE94241.1, YP_{—}004345416.1, EFS79952.1, ZP_{—}06964253.1, EGE60050.1, CBZ52359.1, ADU40304.1, ADQ77229.1, YP_{—}003196038.1, YP_{—}144713.1, YP_{—}001304143.1, YP_{—}113082.1, ADO76516.1, YP_{—}003326349.1, YP_{—}003289755.1, YP_{—}003089327.1, ZP_{—}07911965.1, ZP_{—}05773583.1, ZP_{—}05765271.1, YP_{—}003154888.1, YP_{—}003142045.1, YP_{—}002280953.1, NP_{—}371963.1, NP_{—}422368.1, EGC98966.1, EGC76398.1, YP_{—}004263661.1, YP_{—}004252039.1, YP_{—}679036.1, YP_{—}499973.1, ZP_{—}08090745.1, ZP_{—}08108339.1, YP_{—}001531594.1, ZP_{—}01051588.1, NP_{—}646145.1, NP_{—}224146.1, ZP_{—}08054972.1, ZP_{—}08053009.1, YP_{—}003584878.1, ZP_{—}07939405.1, ZP_{—}03439290.1, ADU82392.1, ADU83943.1, ADU85424.1, ADU80668.1, YP_{—}001225733.1, YP_{—}003863039.1, ZP_{—}01061682.1, YP_{—}767568.1, ZP_{—}07865749.1, ZP_{—}06858058.1, YP_{—}628213.1, EFT81350.1, EFT66610.1, EFT51424.1, ZP_{—}04839161.1, ZP_{—}05633406.1, ZP_{—}05288381.1, AAR37813.1, EFS03282.1, EFS03278.1, YP_{—}004046539.1, ZP_{—}07749550.1, ZP_{—}07729731.1, ADN80650.1, ZP_{—}07088856.1, ZP_{—}07080219.1, ZP_{—}06949721.1, ZP_{—}05685436.1, YP_{—}002550450.1, YP_{—}803715.1, ZP_{—}07720023.1, ZP_{—}07469700.1, ZP_{—}07365619.1, ZP_{—}06924335.1, ZP_{—}06715776.1, ZP_{—}06303722.1, ZP_{—}06303721.1, ZP_{—}06264319.1, ZP_{—}06155528.1, ZP_{—}05745707.1, ZP_{—}04866244.1, ZP_{—}04199629.1, ZP_{—}04195783.1, ZP_{—}04067276.1, ZP_{—}03968868.1, ZP_{—}03963857.1, ZP_{—}03933079.1, ZP_{—}03497046.1, ZP_{—}03475134.1, ZP_{—}01890152.1, ZP_{—}01086712.1, ZP_{—}06021845.1, ZP_{—}02183427.1, ZP_{—}02162695.1, ZP_{—}02032824.1, ZP_{—}01993906.1, ZP_{—}01993127.1, ZP_{—}01983694.1, ZP_{—}01972527.1, ZP_{—}01819838.1, ZP_{—}01817962.1, ZP_{—}01740947.1, ZP_{—}01734991.1, ZP_{—}01694775.1, ZP_{—}01678972.1, ZP_{—}01468566.1, ZP_{—}01408749.1, ZP_{—}01386800.1, ZP_{—}01202184.1, ZP_{—}01174108.1, ZP_{—}01174047.1, ZP_{—}01118729.1, ZP_{—}01081268.1, ZP_{—}00998573.1, ZP_{—}00739793.1, YP_{—}002302140.1, ZP_{—}07358151.1, ZP_{—}06668925.1, ZP_{—}06668924.1, ZP_{—}06667106.1, ZP_{—}06324464.1, ZP_{—}06196777.1, ZP_{—}05114159.1, ZP_{—}05083968.1, ZP_{—}05070370.1, ZP_{—}05030022.1, ZP_{—}04673064.1, ZP_{—}04581752.1, ZP_{—}01052079.1, ZP_{—}07661104.1, ZP_{—}06077819.1, YP_{—}002835579.1, YP_{—}002267069.1, YP_{—}002129114.1, YP_{—}001929236.1, YP_{—}001910999.1, YP_{—}001854051.1, YP_{—}001094152.1, YP_{—}001044252.1, YP_{—}861818.1, YP_{—}915522.1, YP_{—}807371.1, YP_{—}353800.1, YP_{—}342402.1, YP_{—}065168.1, YP_{—}015797.1, YP_{—}005051.1, NP_{—}856449.1, NP_{—}661547.1, NP_{—}358448.1, YP_{—}003929442.1, YP_{—}003927769.1, ADO06185.1, ADO04689.1, ADL23243.1, YP_{—}003789202.1, ADJ79786.1, YP_{—}003516488.1, ADI97953.1, ADI35485.1, YP_{—}003716800.1, ZP_{—}00241359.1, YP_{—}003718040.1, CAQ49862.1, YP_{—}003282331.1, AAP97897.1, ACX99978.1, ACX98578.1, YP_{—}003472544.1, ZP_{—}06382734.1, EEZ79852.1, ZP_{—}05299989.1, ZP_{—}05299895.1, XP_{—}002367632.1, ZP_{—}03529835.1, ZP_{—}03517011.1, ZP_{—}03505783.1, XP_{—}001310698.1, ABK27691.1, CAB59281.2,
in particular NP_{—}391071.1 (encoded by SEQ ID No. 11), BAI86717.1, YP_{—}004205024.1, ZP_{—}06873224.1, YP_{—}003974610.1, YP_{—}001422460.1, AEB25326.1, YP_{—}003921585.1, YP_{—}080482.1, ZP_{—}03054334.1, YP_{—}001488077.1, YP_{—}081348.1, YP_{—}003426902.1, NP_{—}243195.1, ZP_{—}08004522.1, YP_{—}003565624.1, YP_{—}004095847.1, YP_{—}003600348.1, ZP_{—}08006697.1, ZP_{—}04248389.1, YP_{—}174267.1, YP_{—}001376512.1, ZP_{—}04226233.1, ZP_{—}04100460.1, YP_{—}002369417.1, ZP_{—}03229411.1, ZP_{—}04110635.1, ZP_{—}04287684.1, ZP_{—}04172877.1, ZP_{—}04158983.1, ZP_{—}04219330.1, NP_{—}830409.1, YP_{—}003790454.1, ZP_{—}04184510.1, YP_{—}001642542.1, ZP_{—}04074263.1, ZP_{—}04319784.1, NP_{—}847074.1, YP_{—}001373857.1, ZP_{—}04122524.1, ZP_{—}03230841.1, YP_{—}082111.1, NP_{—}834329.1, YP_{—}002444060.1, ZP_{—}04170954.1, YP_{—}002453687.1, ZP_{—}04153266.1, ZP_{—}04302850.1, YP_{—}002365390.1, ZP_{—}04216141.1, ZP_{—}04298961.1, ZP_{—}00740055.1, ZP_{—}04277177.1, ZP_{—}04104350.1, ZP_{—}04176651.1, YP_{—}001647239.1, ZP_{—}04188247.1, ZP_{—}04149717.1, YP_{—}003794343.1, ZP_{—}04230016.1, YP_{—}001643400.1, ZP_{—}04209092.1, ZP_{—}04235899.1, YP_{—}003428808.1, ZP_{—}08005962.1, YP_{—}003599946.1, YP_{—}003565223.1, ZP_{—}01859623.1, YP_{—}004569425.1, ZP_{—}04432601.1, ZP_{—}03227314.1, YP_{—}003699559.1, ZP_{—}07709417.1, ZP_{—}01723571.1, NP_{—}244046.1, ZP_{—}08006365.1, ZP_{—}00738801.1, ZP_{—}04160852.1, ZP_{—}04166021.1, ZP_{—}04154769.1, ZP_{—}04109769.1, ZP_{—}04109049.1, ZP_{—}04108444.1, ZP_{—}04075249.1, ZP_{—}00741173.1, ZP_{—}00739793.1, ZP_{—}01174108.1, ZP_{—}01174047.1, ZP_{—}00241359.1, ZP_{—}04195783.1, ZP_{—}04199629.1, ZP_{—}04067276.1
and particularly preferably NP_{—}391071.1 (encoded by SEQ ID No. 11)
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E₃ in general is understood to mean in particular the conversion of pyruvate to alanine.

### Enzyme E₄ ω-Oxidation

For the production of an ω-amino-functionalized carboxylic acid or an ω-amino-functionalized carboxylate ester, it can be advantageous if the second genetic modification comprises increased activity of an enzyme E₄ which catalyses the conversion of ω-hydroxycarboxylic acids or ω-hydroxycarboxylate esters to the corresponding ω-oxocarboxylic acids or ω-oxocarboxylate esters. This increased activity of the enzyme E₄ can also be advantageous if the preparation of ω-oxocarboxylic acids, ω-oxocarboxylate esters, ω-carboxycarboxylic acid or ω-carboxycarboxylate esters is desired.

Should the microorganisms according to the invention be used in a process for the production of ω-oxocarboxylic acids or ω-oxocarboxylate esters or of ω-functionalized compounds derived from ω-oxocarboxylic acids or ω-oxocarboxylate esters such as for example ω-amino compounds, then it is advantageous if the microorganism, as already described above for E₂, displays decreased activity of an aldehyde dehydrogenase of EC 1.2.1.3, EC 1.2.1.4 or EC 1.2.1.5 in comparison to its wild type. In this connection, preferred enzymes E₄ are those which only catalyse the respective first-mentioned of the two reactions mentioned in the following section.

### Specific Enzymes E₄

Preferably the enzyme E₄ is

a fatty alcohol oxidase of EC 1.1.3.20, which preferentially catalyses at least one of the following reactions, in particular the first-mentioned:
ω-hydroxyalkanoic acid (ester)+O₂=ω-oxoalkanoic acid (ester)+H₂O₂ and ω-oxoalkanoic acid (ester)+O₂=ω-carboxyalkanoic acid (ester)+H₂O₂ or an AlkJ alcohol dehydrogenase of EC 1.1.99.-, which preferentially catalyses at least one of the following reactions, in particular the first-mentioned:
ω-hydroxyalkanoic acid (ester)+oxidized acceptor=ω-oxoalkanoic acid (ester)+reduced acceptor and
ω-oxoalkanoic acid (ester)+oxidized acceptor=ω-carboxyalkanoic acid (ester)+reduced acceptor or
an alcohol dehydrogenase of EC 1.1.1.1 or EC 1.1.1.2, which preferentially catalyses at least one of the following reactions, in particular the first-mentioned:
ω-hydroxyalkanoic acid (ester)+NAD(P)⁺=ω-oxoalkanoic acid (ester)+NAD(P)H+ H⁺ and
ω-oxoalkanoic acid (ester)+NAD(P)⁺=ω-carboxyalkanoic acid (ester)+NAD(P)H+H⁺.

Such preferable fatty alcohol oxidases are selected from

AAS46878.1, ACX81419.1, AAS46879.1, CAB75353.1, AAS46880.1, XP_{—}712350.1, XP_{—}002422236.1, XP_{—}712386.1, EEQ43775.1, XP_{—}001525361.1, XP_{—}001386087.1, XP_{—}459506.2, CAB75351.1, CAB75352.1, XP_{—}001385255.2, EDK39369.2, XP_{—}001484086.1, XP_{—}002618046.1, XP_{—}002548766.1, XP_{—}002548765.1, XP_{—}003041566.1, XP_{—}003328562.1, XP_{—}001214264.1, XP_{—}001904377.1, XP_{—}658227.1, XP_{—}001591990.1, XP_{—}753079.1, XP_{—}002569337.1, XP_{—}001268562.1, XP_{—}003348911.1, EGP90120.1, XP_{—}001389382.1, EER37923.1, XP_{—}001264046.1, EGO58212.1, XP_{—}001554225.1, XP_{—}003298648.1, XP_{—}959005.1, XP_{—}002841296.1, XP_{—}001940486.1, EGR52262.1, EEQ89581.1, EGD99881.1, EFQ33355.1, XP_{—}001821106.1, XP_{—}002622231.1, EGG03784.1, EGC44059.1, XP_{—}003018036.1, XP_{—}003011696.1, EFY90752.1, XP_{—}001227812.1, XP_{—}758170.1, XP_{—}001243546.1, XP_{—}002479333.1, XP_{—}003344707.1, EFW14100.1, XP_{—}003071927.1, XP_{—}003171263.1, XP_{—}003051757.1, XP_{—}002147053.1, EEH19591.1, EEH50473.1, XP_{—}001792978.1, XP_{—}387094.1, EFY98644.1, XP_{—}002788971.1, XP_{—}002842592.1, EFX04185.1, XP_{—}003231449.1, XP_{—}001729067.1, CBX94189.1, XP_{—}001413535.1, ACF22878.1, B5WWZ9.1, XP_{—}002994642.1, XP_{—}002269629.1, XP_{—}002519938.1, XP_{—}002982582.1, NP_{—}001047464.1, EEC73620.1, XP_{—}002981110.1, XP_{—}002960521.1, NP_{—}566729.1, XP_{—}001541970.1, XP_{—}002967201.1, BAK00483.1, XP_{—}002182547.1, BAK02336.1, XP_{—}002454190.1, XP_{—}002328753.1, XP_{—}002867943.1, XP_{—}002285334.1, CAC87643.1, CAN71289.1, XP_{—}002454188.1, AAL31049.1, XP_{—}002464494.1, AAL31021.1, YP_{—}117187.1, XP_{—}002543430.1, CAA18625.1, XP_{—}002883430.1, NP_{—}193673.2, XP_{—}002529832.1, XP_{—}001753124.1, NP_{—}001142399.1, ACN27562.1, XP_{—}002464495.1, ACR36691.1, BAJ86655.1, B5WWZ8.1, NP_{—}001148058.1, ABR17814.1, EAY78905.1, NP_{—}194586.1, AAM63097.1, AAK64154.1, NP_{—}001064839.2, XP_{—}002869492.1, XP_{—}002314488.1, AAL31024.1, ZP_{—}06967355.1, AAP54248.2, XP_{—}002311685.1, ACF87929.1, YP_{—}907078.1, EGE07035.1, YP_{—}001849908.1, XP_{—}002464496.1, EEC67160.1, AAL31027.1, XP_{—}001761391.1, XP_{—}002961172.1, XP_{—}002528823.1, XP_{—}002966834.1, NP_{—}001176205.1, XP_{—}001763007.1, XP_{—}002272123.1, XP_{—}002889487.1, XP_{—}003003157.1, NP_{—}285451.1, EGG23219.1, NP_{—}171895.2, YP_{—}003395677.1, Q9ZWB9.1, ACF88407.1, ZP_{—}06413771.1, EEE51131.1, YP_{—}003835264.1, YP_{—}003397164.1, YP_{—}004081922.1, XP_{—}003294587.1, EEE51130.1, YP_{—}003647529.1, YP_{—}003647985.1, CBI29206.3, XP_{—}629786.1, ZP_{—}07964664.1, EEE57396.1, EEH09589.1, YP_{—}003265796.1, YP_{—}001840752.1, ZP_{—}08620775.1, ACR36076.1, ZP_{—}05043749.1, YP_{—}980677.1, ZP_{—}05043728.1, YP_{—}692894.1, NP_{—}710223.1, EEC67159.1, AAP03110.1, EFA85697.1, YP_{—}691805.1, YP_{—}551012.1, YP_{—}001174466.1, YP_{—}002796294.1, YP_{—}004716331.1, YP_{—}001019547.1, YP_{—}585737.1, AEA86007.1, YP_{—}960830.1, YP_{—}004743970.1, ZP_{—}03431349.1, ZP_{—}06448642.1, ZP_{—}07430351.1, NP_{—}215006.2, ZP_{—}03535393.1, ZP_{—}06801690.1, YP_{—}001849132.1, NP_{—}854165.1, ZP_{—}03427234.1, CBJ27378.1, NP_{—}334920.1, ZP_{—}08571383.1, YP_{—}728161.1, ZP_{—}01896040.1, ZP_{—}03530923.1, YP_{—}551306.1, YP_{—}003167456.1, YP_{—}606070.1, ZP_{—}06850167.1, ADP99095.1, YP_{—}907986.1, ZP_{—}04924166.1, ZP_{—}08139923.1, YP_{—}001270300.1, YP_{—}521830.1, YP_{—}003147410.1, YP_{—}002007173.1, ADR62464.1, YP_{—}004382294.1, NP_{—}747223.1, YP_{—}004687462.1, NP_{—}902159.1, ZP_{—}04936784.1, YP_{—}003914667.1, ZP_{—}01306356.1, ZP_{—}04750553.1, YP_{—}002875279.1, YP_{—}004704374.1, YP_{—}001671392.1, NP_{—}249055.1, ZP_{—}06876360.1, YP_{—}001345853.1, YP_{—}002437969.1, YP_{—}004356853.1, YP_{—}351075.1, CBI23676.3, YP_{—}001189668.1, YP_{—}001528881.1, YP_{—}001613612.1, YP_{—}001747218.1, YP_{—}003393002.1, YP_{—}001365074.1, ZP_{—}07778129.1, ZP_{—}07392715.1, YP_{—}001553329.1, YP_{—}262925.1, YP_{—}751961.1, YP_{—}564183.1, YP_{—}003811876.1, YP_{—}002356821.1, YP_{—}001051828.1, YP_{—}001837525.1, NP_{—}716513.1, ZP_{—}01915079.1, ZP_{—}02156621.1, YP_{—}001184631.1, YP_{—}001475595.1, ZP_{—}05042393.1, YP_{—}962228.1, YP_{—}001612275.1, ADV55625.1, YP_{—}001675797.1, YP_{—}003555260.1, ZP_{—}01075039.1, YP_{—}003812822.1, YP_{—}001503351.1, EFN52938.1, YP_{—}001759063.1, ZP_{—}06503577.1, YP_{—}871025.1, ZP_{—}08564919.1, YP_{—}002310162.1, YP_{—}732875.1, YP_{—}001092722.1, YP_{—}739324.1, XP_{—}002333995.1, NP_{—}085596.1, YP_{—}928870.1, EGD05748.1, NP_{—}443993.1, ZP_{—}08138057.1, ZP_{—}05041587.1, ZP_{—}07011380.1, YP_{—}001612684.1, ZP_{—}07669342.1, ZP_{—}06508361.1, ZP_{—}03423639.1, YP_{—}923293.1, ZP_{—}05061865.1, ZP_{—}08181496.1, YP_{—}559605.1, ZP_{—}06841320.1, ZP_{—}01620712.1, YP_{—}001896340.1, ZP_{—}03276650.1, YP_{—}004303194.1, ZP_{—}08180715.1, ZP_{—}06382740.1, ZP_{—}01034555.1, YP_{—}004604560.1, YP_{—}001020142.1, YP_{—}935375.1, ZP_{—}01546137.1, ZP_{—}07661079.1, YP_{—}001860640.1, ZP_{—}06052841.1, ZP_{—}01881170.1, ZP_{—}05781455.1, YP_{—}932732.1, ZP_{—}08119300.1, YP_{—}004715268.1, ZP_{—}03697402.1, YP_{—}004126957.1, ZP_{—}06703136.1, NP_{—}642445.1, ZP_{—}08273900.1, YP_{—}004524313.1, ZP_{—}01902993.1, YP_{—}001900094.1, AEA84888.1, YP_{—}004690289.1, NP_{—}714358.1, YP_{—}682471.1, YP_{—}003239.1, YP_{—}997465.1, YP_{—}003452130.1, ZP_{—}01739153.1, YP_{—}004219483.1, YP_{—}001761298.1, ZP_{—}01438251.1, CBI37146.3, ZP_{—}04748383.1, YP_{—}004362245.1, ZP_{—}05912795.1, YP_{—}003390234.1, YP_{—}003122799.1, CCB77579.1, EGB06416.1, ZP_{—}08389346.1, YP_{—}191496.1, ZP_{—}05224727.1, ZP_{—}01125614.1, YP_{—}466287.1, YP_{—}001368620.1, YP_{—}001380256.1, YP_{—}002361951.1, YP_{—}002756103.1, YP_{—}001801399.1, ZP_{—}06847140.1, YP_{—}003200069.1, YP_{—}001940247.1, YP_{—}001584322.1, ZP_{—}04679227.1, YP_{—}002493674.1, YP_{—}002135530.1, YP_{—}004290424.1, YP_{—}001772011.1, ZP_{—}08189046.1, ZP_{—}03423640.1, YP_{—}001834251.1, ZP_{—}01041752.1, YP_{—}001533410.1, YP_{—}269751.1, YP_{—}002432994.1, YP_{—}003694653.1, CAD47896.1, NP_{—}769359.1, YP_{—}004239460.1, YP_{—}004605221.1, YP_{—}001961214.1, YP_{—}001837513.1, YP_{—}004335962.1, YP_{—}004358600.1, ZP_{—}05050026.1, YP_{—}003202983.1, BAD03777.1, ZP_{—}02165013.1, NP_{—}774131.1, YP_{—}432169.1, ZP_{—}05000547.1, YP_{—}001261233.1, XP_{—}002593969.1, XP_{—}002603265.1, YP_{—}003342435.1, ZP_{—}01253183.1, EGO36831.1, YP_{—}001866737.1, YP_{—}001523879.1, YP_{—}133594.1, YP_{—}003768990.1, YP_{—}001237820.1, YP_{—}003133224.1, ZP_{—}01896771.1, ZP_{—}01865125.1, NP_{—}960319.1, YP_{—}826958.1, YP_{—}003326608.1, YP_{—}002219515.1, NP_{—}217926.1, ZP_{—}07441899.2, YP_{—}001208178.1, ADM42038.1, YP_{—}002433510.1, ZP_{—}08274313.1, EGO38668.1, ZP_{—}03393221.1, NP_{—}356358.1, ZP_{—}06055780.1, YP_{—}001684562.1, ZP_{—}08528157.1, BAD03162.1, YP_{—}001800712.1, ACL37106.1, YP_{—}883489.1, ZP_{—}01075202.1, NP_{—}969446.1, ZP_{—}01129577.1, YP_{—}001530285.1, ZP_{—}04746501.1, YP_{—}001341980.1, YP_{—}905003.1, ZP_{—}05218299.1, ZP_{—}08665577.1,
preferably
AAS46878.1, ACX81419.1, AAS46879.1, CAB75353.1, AAS46880.1, XP_{—}712350.1, XP_{—}002422236.1, XP_{—}712386.1, EEQ43775.1, XP_{—}001525361.1, XP_{—}001386087.1, XP_{—}459506.2, CAB75351.1, CAB75352.1, XP_{—}001385255.2, EDK39369.2, XP_{—}001484086.1, XP_{—}002618046.1, XP_{—}002548766.1, XP_{—}002548765.1, XP_{—}003041566.1, XP_{—}001214264.1, XP_{—}001904377.1, XP_{—}658227.1, XP_{—}001591990.1, XP_{—}753079.1, XP_{—}002569337.1, XP_{—}001268562.1, XP_{—}003348911.1, EGP90120.1, XP_{—}001389382.1, EER37923.1, XP_{—}001264046.1, EGO58212.1, XP_{—}001554225.1, XP_{—}003298648.1, XP_{—}959005.1, XP_{—}002841296.1, XP_{—}001940486.1, EGR52262.1, EEQ89581.1, EGD99881.1, EFQ33355.1, XP_{—}001821106.1, XP_{—}002622231.1, EGC44059.1, XP_{—}003018036.1, XP_{—}003011696.1, EFY90752.1, XP_{—}001227812.1, XP_{—}001243546.1, XP_{—}002479333.1, XP_{—}003344707.1, EFW14100.1, XP_{—}003071927.1, XP_{—}003171263.1, XP_{—}003051757.1, XP_{—}002147053.1, EEH19591.1, EEH50473.1, XP_{—}001792978.1, XP_{—}387094.1, EFY98644.1, XP_{—}002788971.1, XP_{—}002842592.1, EFX04185.1, XP_{—}003231449.1, CBX94189.1, XP_{—}001413535.1, XP_{—}001541970.1, XP_{—}002543430.1, EGE07035.1, XP_{—}003003157.1
and particularly preferably

### AAS46878.1, ACX81419.1, AAS46879.1, CAB75353.1, AAS46880.1, XP_{—}712350.1, XP_{—}002422236.1, XP_{—}712386.1, EEQ43775.1, CAB75351.1, CAB75352.1, XP_{—}002548766.1, XP_{—}002548765.1,

and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E₄ in general is understood to mean in particular the conversion of ω-oxolauric acid and/or methyl ω-oxolaurate to ω-carboxylauric acid and/or methyl w-carboxylaurate or the conversion of ω-hydroxylauric acid and/or methyl ω-hydroxylaurate to w-oxolauric acid and/or methyl ω-oxolaurate.

Such preferable AlkJ alcohol dehydrogenases are selected from

Q00593.1, Q9WWW2.1, ZP_{—}00957061.1, YP_{—}957894.1, CAC38030.1, YP_{—}694430.1, YP_{—}957725.1, YP_{—}001672216.1, YP_{—}552061.1, YP_{—}130410.1, ZP_{—}06155535.1, ZP_{—}01222730.1, YP_{—}691907.1, YP_{—}002297804.1, YP_{—}004283522.1, YP_{—}001234383.1, YP_{—}004435031.1, ZP_{—}05110316.1, ZP_{—}05042898.1, YP_{—}004466324.1, ZP_{—}08553549.1, YP_{—}004125220.1, ADI22536.1, ADI18461.1, YP_{—}003810975.1, YP_{—}662346.1, YP_{—}004427557.1, YP_{—}692606.1, ZP_{—}05043291.1, YP_{—}440752.1, ZP_{—}02386160.1, ZP_{—}04763547.1, ZP_{—}02361232.1, YP_{—}003376674.1, ZP_{—}02354055.1, ZP_{—}05085930.1, ADQ00130.1, YP_{—}003643016.1, ZP_{—}05040520.1, YP_{—}691922.1, AAX23098.1, BAD07371.1, NP_{—}104379.1, YP_{—}002551960.1, YP_{—}003908558.1, YP_{—}987903.1, ZP_{—}05785860.1, YP_{—}004145612.1, YP_{—}004140926.1, CAZ88300.1, ZP_{—}05041901.1, YP_{—}533645.1, ZP_{—}01754259.1, CBA31223.1, YP_{—}87542.1, YP_{—}106852.1, ZP_{—}08402506.1, ZP_{—}05055020.1, ZP_{—}02400829.1, YP_{—}104747.1, ZP_{—}02409412.1, YP_{—}001057269.1, YP_{—}004229837.1, YP_{—}294429.1, YP_{—}001028112.1, ZP_{—}02479747.1, YP_{—}002874799.1, ZP_{—}03541051.1, YP_{—}003606536.1, ZP_{—}02887167.1, YP_{—}001795572.1, YP_{—}487451.1, ACZ62814.1, YP_{—}560809.1, ZP_{—}02167462.1, YP_{—}004482869.1, YP_{—}001581248.1, ZP_{—}07374066.1, YP_{—}001203981.1, ZP_{—}06840259.1, ZP_{—}01915145.1, NP_{—}774525.1, ZP_{—}03561080.1, YP_{—}001208258.1, YP_{—}001897374.1, YP_{—}001413909.1, YP_{—}366469.1, YP_{—}521854.1, YP_{—}004490642.1, YP_{—}003280349.1, ZP_{—}03588744.1, YP_{—}001562229.1, YP_{—}001120981.1, ZP_{—}03574970.1, YP_{—}004234225.1, ZP_{—}02377531.1, ZP_{—}02149954.1, YP_{—}001237360.1, ZP_{—}03266156.1, YP_{—}782821.1, YP_{—}004754039.1, BAB61732.1, ZP_{—}07046388.1, ZP_{—}02145452.1, BAF45123.1, YP_{—}002129953.1, YP_{—}003812439.1, ZP_{—}01055291.1, BAF45124.1, EGH71399.1, ZP_{—}05060389.1, ZP_{—}05090872.1, BAF45126.1, BAB07804.1, ZP_{—}06053464.1, YP_{—}001238278.1, ZP_{—}04944469.1, YP_{—}001171160.1, YP_{—}002984373.1, YP_{—}002237649.1, ZP_{—}08276443.1, BAF98451.1, ZP_{—}05124197.1, YP_{—}568640.1, ZP_{—}05785341.1, NP_{—}769037.1, YP_{—}370657.1, YP_{—}775005.1, ZP_{—}02911119.1, YP_{—}165460.1, ZP_{—}02891796.1, YP_{—}622328.1, ZP_{—}07675057.1, YP_{—}001901188.1, YP_{—}003592183.1, ZP_{—}02361040.1, NP_{—}518244.1, YP_{—}001809673.1, NP_{—}947032.1, YP_{—}001766369.1, YP_{—}002255997.1, ZP_{—}04940241.1, YP_{—}004012032.1, YP_{—}841049.1, YP_{—}002983249.1, YP_{—}003643276.1, YP_{—}003855487.1, YP_{—}003778137.1, ZP_{—}02361104.1, CBA30511.1, ZP_{—}05781295.1, YP_{—}756865.1, ZP_{—}02461782.1, YP_{—}002007988.1, YP_{—}004110133.1, YP_{—}002229680.1, ZP_{—}02386040.1, YP_{—}004684069.1, YP_{—}373268.1, YP_{—}440614.1, NP_{—}421441.1, YP_{—}264896.1, YP_{—}004362617.1, ZP_{—}06053847.1, YP_{—}366538.1, YP_{—}003812285.1, YP_{—}004154520.1, ZP_{—}01901081.1, ZP_{—}02372179.1, ZP_{—}02453559.1, ADP98564.1, YP_{—}003747084.1, ZP_{—}02487888.1, ZP_{—}01768075.1, ZP_{—}02400664.1, YP_{—}106680.1, YP_{—}724753.1, YP_{—}002907583.1, YP_{—}004482470.1, YP_{—}167582.1, YP_{—}270109.1, YP_{—}004362333.1, ZP_{—}02504034.1, YP_{—}003189363.1, YP_{—}973212.1, ZP_{—}00952746.1, YP_{—}459665.1, YP_{—}777218.1, YP_{—}581107.1, ZP_{—}01878091.1, ZP_{—}01057973.1, YP_{—}002913124.1, ZP_{—}01035570.1, YP_{—}001777560.1, YP_{—}552627.1, ZP_{—}02890876.1, YP_{—}587146.1, YP_{—}004141814.1, YP_{—}001685369.1, ZP_{—}05343380.1, NP_{—}886000.1, ZP_{—}04942359.1, ZP_{—}01913732.1, ZP_{—}08244266.1, YP_{—}002233254.1, ZP_{—}01816670.1, YP_{—}837233.1, ZP_{—}07478008.1, ZP_{—}01985205.1, ZP_{—}07473972.1, ZP_{—}01067090.1, ZP_{—}01867788.1, ZP_{—}01754024.1, EGM19144.1, ZP_{—}07741283.1, ZP_{—}06876839.1, YP_{—}002395287.1, ZP_{—}07795498.1, NP_{—}102692.1, NP_{—}252789.1, YP_{—}004451100.1, ZP_{—}01305514.1, YP_{—}002438481.1, ZP_{—}04930310.1, YP_{—}001810189.1, YP_{—}104187.1, ZP_{—}01367534.1, YP_{—}001346382.1, ZP_{—}01878466.1, YP_{—}789017.1, YP_{—}001115422.1, ZP_{—}05067451.1, ZP_{—}05842072.1, YP_{—}001682976.1, YP_{—}761348.1, YP_{—}004611600.1, YP_{—}004188241.1, NP 419761.1, EFV85163.1, YP_{—}684227.1, ZP_{—}06177455.1, NP_{—}935088.1, YP_{—}004614491.1, ZP_{—}08697916.1, YP_{—}004689366.1, ZP_{—}05052326.1, YP_{—}267420.1, YP_{—}728575.1, YP_{—}001759584.1, YP_{—}557446.1, ZP_{—}06844897.1, ZP_{—}06079799.1, YP_{—}003771143.1, ZP_{—}05094472.1, YP_{—}511622.1, ACF98205.1, YP_{—}582314.1, ZP_{—}07660450.1, YP_{—}004065269.1, YP_{—}003979606.1, YP_{—}002520401.1, YP_{—}003579281.1, ZP_{—}01749397.1, ZP_{—}03265018.1, ZP_{—}07283393.1, YP_{—}001532150.1, YP_{—}298941.1, ZP_{—}06688181.1, ZP_{—}01611660.1, ZP_{—}02367747.1, EGP42870.1, ZP_{—}00993245.1, ABY65992.1, YP_{—}354800.1, ZP_{—}01747277.1, YP_{—}561728.1, ZP_{—}02190947.1, YP_{—}605824.1, YP_{—}001991873.1, ZP_{—}00955792.1, YP_{—}003594401.1, YP_{—}004156101.1, YP_{—}001472858.1, YP_{—}001746950.1, ZP_{—}08410042.1, ZP_{—}01116604.1, ADP99912.1, ZP_{—}01692203.1, YP_{—}001328534.1, YP_{—}999236.1, YP_{—}002278452.1, ZP_{—}01306234.1, YP_{—}002871776.1, ZP_{—}02369920.1, ZP_{—}01896942.1, YP_{—}002289724.1, AEG07584.1, YP_{—}999005.1, YP_{—}003552461.1, YP_{—}270668.1, ZP_{—}06862917.1, YP_{—}001811327.1, YP_{—}001166036.1, ABW06653.1, ZP_{—}01548976.1, ZP_{—}07774606.1, ZP_{—}05888080.1, YP_{—}003301477.1, YP_{—}341748.1, ZP_{—}05100248.1, YP_{—}918038.1, YP_{—}001500869.1, YP_{—}004305296.1, YP_{—}003342584.1, NP_{—}947961.1, ZP_{—}05124765.1, ZP_{—}01904700.1, YP_{—}003696207.1, YP_{—}004156699.1, YP_{—}001241858.1, NP_{—}104253.1, YP_{—}676241.1, ZP_{—}01736903.1, ZP_{—}00960121.1, NP_{—}436019.1, YP_{—}002945716.1, YP_{—}259594.1, EFV86615.1, AAY87334.1, NP_{—}900970.1, AEG07409.1, YP_{—}349087.1, YP_{—}004141055.1, YP_{—}001169476.1, YP_{—}001566960.1, YP_{—}260472.1, ZP_{—}07028078.1, YP_{—}004610468.1, YP_{—}003066461.1, YP_{—}961096.1, ZP_{—}08666573.1, ZP_{—}02187363.1, YP_{—}001631518.1, ZP_{—}08141293.1, YP_{—}001666324.1, NP_{—}387083.1, YP_{—}001526184.1, YP_{—}165213.1, YP_{—}003694923.1, YP_{—}004433897.1, YP_{—}001265431.1, ZP_{—}05068964.1, YP_{—}002313077.1, ZP_{—}02372305.1, YP_{—}004486039.1, YP_{—}341901.1, YP_{—}001862312.1, YP_{—}004681983.1, YP_{—}617373.1, EFV86570.1, YP_{—}001673285.1, BAK39604.1, YP_{—}001669327.1, YP_{—}004353150.1, YP_{—}001888124.1, ZP_{—}08645365.1, YP_{—}003410784.1, YP_{—}841363.1, EGP44033.1, YP_{—}001633470.1, EGP42855.1, ZP_{—}01115125.1, ADR57794.1, YP_{—}784649.1, YP_{—}373898.1, Q47944.1, YP_{—}001117950.1, ZP_{—}02380339.1, ZP_{—}03697092.1, YP_{—}003187112.1, YP_{—}004065439.1, NP_{—}742226.1, YP_{—}002429878.1, YP_{—}003556403.1, AEH81535.1, YP_{—}001887935.1, YP_{—}554605.1, ZP_{—}07333059.1, YP_{—}001991668.1, YP_{—}003694210.1, YP_{—}222680.1, YP_{—}002232672.1, YP_{—}001763402.1, YP_{—}001806802.1, YP_{—}662156.1, ZP_{—}05153429.1, ZP_{—}01893457.1, ZP_{—}04595387.1, ADP99389.1, ZP_{—}02890074.1, YP_{—}001313582.1, NP_{—}387401.1, ZP_{—}01863693.1, YP_{—}750630.1, ZP_{—}04939997.1, YP_{—}268077.1, ZP_{—}05169265.1, NP_{—}888994.1, ZP_{—}08408421.1, YP_{—}001155137.1, NP_{—}699017.1, YP_{—}002008190.1, YP_{—}004493716.1, YP_{—}266277.1, YP_{—}004654190.1, YP_{—}943422.1, ZP_{—}05162503.1, ZP_{—}02905080.1, ZP_{—}02905080.1, ZP_{—}03784461.1, YP_{—}001601784.1, YP_{—}002233786.1, YP_{—}622842.1, YP_{—}002822679.1, ZP_{—}04944312.1, ZP_{—}05179897.1, YP_{—}004483124.1, YP_{—}003390414.1, YP_{—}771968.1, YP_{—}001628465.1, YP_{—}004311599.1, ZP_{—}01037150.1, ZP_{—}01611812.1, ZP_{—}03575238.1, YP_{—}002278603.1, YP_{—}001593845.1, EGD01613.1, YP_{—}297574.1, YP_{—}367509.1, YP_{—}998315.1, ZP_{—}08664883.1, ZP_{—}05114787.1, ZP_{—}05450190.1, YP_{—}298028.1, ZP_{—}01034678.1, YP_{—}002827796.1, YP_{—}372762.1, YP_{—}004466723.1, ZP_{—}01012072.1, YP_{—}320380.1, ZP_{—}01075202.1, YP_{—}001312358.1, YP_{—}681895.1, ZP_{—}07718189.1, EGP55868.1, YP_{—}003750799.1, YP_{—}002984725.1, YP_{—}002543360.1, ZP_{—}01040714.1, ZP_{—}04717111.1, YP_{—}002422932.1, YP_{—}003506115.1, ZP_{—}01444019.1, ZP_{—}03587285.1, YP_{—}771439.1, YP_{—}001947593.1, YP_{—}001049712.1, YP_{—}003979888.1, YP_{—}001553786.1, YP_{—}003980878.1, YP_{—}001578274.1, YP_{—}472442.1, YP_{—}778292.1, EGE56670.1, YP_{—}002779312.1, YP_{—}432169.1, YP_{—}560963.1, YP_{—}001265285.1, YP_{—}002822699.1, YP_{—}002278091.1, ZP_{—}08632361.1, YP_{—}002229178.1, ZP_{—}06840392.1, ZP_{—}05069105.1, ZP_{—}00998644.1, YP_{—}004487901.1, YP_{—}680905.1, YP_{—}728088.1, YP_{—}001985833.1, YP_{—}002007099.1, ZP_{—}05066777.1, ZP_{—}01551182.1, YP_{—}002973332.1, ZP_{—}04681414.1, ZP_{—}07675148.1, AEH83964.1, YP_{—}004692042.1, CBJ36337.1, EGP48473.1, ZP_{—}03585612.1, YP_{—}001369428.1, YP_{—}001897527.1, AEG08472.1, YP_{—}001166065.1, NP_{—}437018.1, NP_{—}294689.1, YP_{—}002541437.1, YP_{—}004692953.1, NP_{—}107484.1, YP_{—}995681.1, YP_{—}765267.1, YP_{—}166223.1, ZP_{—}01740635.1, YP_{—}001234127.1, ZP_{—}02186681.1, YP_{—}004140839.1, YP_{—}001584499.1, ADI17244.1, ZP_{—}08698744.1, YP_{—}001022991.1, EFV84582.1, ZP_{—}01743515.1, YP_{—}001816113.1, YP_{—}004688050.1, YP_{—}001342912.1, ZP_{—}01125614.1, EGD05029.1, ZP_{—}03569823.1, ZP_{—}05089337.1, YP_{—}001901091.1, NP_{—}886663.1, ZP_{—}07718907.1, YP_{—}004687387.1, NP_{—}521464.1, ZP_{—}06688394.1, ZP_{—}08099738.1, ZP_{—}02885452.1, YP_{—}003744085.1, YP_{—}001328823.1, ZP_{—}02488044.1, ZP_{—}01015005.1, YP_{—}002983153.1, ZP_{—}06898725.1, ZP_{—}05886707.1, ZP_{—}08101209.1, ZP_{—}03319462.1, YP_{—}003134969.1, YP_{—}001188857.1, YP_{—}004557767.1, YP_{—}004675666.1, YP_{—}004358728.1, YP_{—}002252541.1, YP_{—}684009.1, ZP_{—}05085667.1, ZP_{—}02144674.1, YP_{—}004127560.1, ZP_{—}01901604.1, YP_{—}004280074.1, AEG67402.1, YP_{—}001416516.1, ZP_{—}01054720.1, ZP_{—}08197897.1, NP_{—}107235.1, YP_{—}002909966.1, ZP_{—}01545876.1, ZP_{—}02147729.1, ZP_{—}00946537.1, ZP_{—}01903844.1, ZP_{—}05085589.1, ACV84069.1, YP_{—}367172.1, ZP_{—}02165272.1, YP_{—}701696.1, ZP_{—}04935724.1, ZP_{—}02191362.1, ZP_{—}01740154.1, ZP_{—}07662819.1, NP_{—}103908.1, YP_{—}003159313.1, YP_{—}003197010.1, ZP_{—}02152342.1, YP_{—}001907189.1, YP_{—}004387414.1, YP_{—}001413869.1, ZP_{—}01916549.1, ZP_{—}03264661.1, AAY82840.1, YP_{—}003277969.1, YP_{—}767433.1, ZP_{—}01226234.1, EGE55950.1, NP_{—}882474.1, ZP_{—}04680938.1, YP_{—}004417965.1, ZP_{—}01367142.1, EGM13684.1, YP_{—}001262083.1, ZP_{—}01881606.1, ZP_{—}01002680.1, YP_{—}003606679.1, YP_{—}001868359.1, ZP_{—}01446736.1, YP_{—}004141411.1, YP_{—}002438878.1, YP_{—}002500414.1, EGP55675.1, ZP_{—}08405873.1, YP_{—}002975318.1, YP_{—}002823637.1, ZP_{—}02188786.1, YP_{—}004617386.1, ABL61001.1, YP_{—}004190679.1, YP_{—}004418710.1, YP_{—}001264994.1, NP_{—}252399.1, ACA21517.1, YP_{—}002541208.1, YP_{—}001369943.1, YP_{—}789454.1, YP_{—}004688060.1, YP_{—}611623.1, ZP_{—}07795086.1, ZP_{—}04929943.1, YP_{—}004444316.1, ZP_{—}01866687.1, ZP_{—}05973466.1, YP_{—}004353327.1, ZP_{—}05780591.1, ZP_{—}05784784.1, NP_{—}936564.1, ZP_{—}05739211.1, ZP_{—}05113045.1, ZP_{—}06689273.1, ZP_{—}06972168.1, ZP_{—}01616404.1, ZP_{—}07659253.1, ZP_{—}05117914.1, YP_{—}585662.1, YP_{—}004230016.1, NP_{—}763554.1, NP_{—}744101.1, ZP_{—}02465308.1, ACN56476.1, YP_{—}004689565.1, YP_{—}001600608.1, ZP_{—}06792595.1, YP_{—}001258553.1, ZP_{—}05165722.1, ZP_{—}03785098.1, YP_{—}002276744.1, YP_{—}002524856.1, ADP98420.1, YP_{—}001669248.1, ZP_{—}04764988.1, ZP_{—}08528163.1, ZP_{—}08529409.1, ZP_{—}05944625.1, YP_{—}676267.1, CBA26630.1, YP_{—}001592413.1, YP_{—}003486465.1, ZP_{—}02187562.1, ZP_{—}03702891.1, YP_{—}760283.1, ZP_{—}05450850.1, YP_{—}004533595.1, ZP_{—}02153313.1, YP_{—}001859265.1, YP_{—}001524099.1, ZP_{—}06126913.1, ZP_{—}07374926.1, ZP_{—}05050787.1, ZP_{—}01035411.1, Q8YFY2.2, YP_{—}002280903.1, EGM21512.1, YP_{—}004603010.1, ZP_{—}05088581.1, YP_{—}004302488.1, YP_{—}004141219.1, NP_{—}697569.1, YP_{—}003908705.1, YP_{—}915505.1, YP_{—}001789228.1, YP_{—}001042739.1, YP_{—}133405.1, ZP_{—}05180516.1, ZP_{—}05174702.1, ZP_{—}01438051.1, ZP_{—}04590345.1, ZP_{—}08411937.1, NP_{—}356519.2, ZP_{—}00964019.1, ZP_{—}00998343.1, ZP_{—}05181994.1, YP_{—}004107969.1, ZP_{—}02168070.1, ZP_{—}01750865.1, YP_{—}574504.1, YP_{—}004579902.1, YP_{—}104440.1, ZP_{—}05452167.1, ZP_{—}05342702.1, YP_{—}001862883.1, YP_{—}004538242.1, ZP_{—}07471513.1, ZP_{—}05169558.1, ZP_{—}00956995.1, ZP_{—}05096699.1, YP_{—}004610916.1, ZP_{—}01218118.1, AAU95210.1, ZP_{—}02405087.1, ZP_{—}04890639.1, YP_{—}352237.1, ZP_{—}02413594.1, ZP_{—}07474023.1, NP_{—}541317.1, YP_{—}001993222.1, ZP_{—}08199001.1, YP_{—}471839.1, ZP_{—}02492080.1, ZP_{—}04901176.1, ZP_{—}06915396.1, ZP_{—}07474845.1, ZP_{—}07477743.1, YP_{—}004152647.1, YP_{—}004755056.1, ZP_{—}05086419.1, YP_{—}004577547.1, ACD99850.1, YP_{—}980426.1, ZP_{—}05457072.1, ZP_{—}05936041.1, NP_{—}700124.1, ADT85599.1, YP_{—}110012.1, ZP_{—}05076113.1, YP_{—}001068288.1, ZP_{—}02457871.1, ZP_{—}01014169.1, EGE60620.1, YP_{—}001346810.1, YP_{—}003408795.1, YP_{—}003769675.1, YP_{—}001257876.1, EGH93583.1, ZP_{—}01442222.1, YP_{—}331617.1, ZP_{—}05636703.1, YP_{—}001594896.1, YP_{—}002822967.1, YP_{—}118823.1, ZP_{—}01878717.1, ZP_{—}07375284.1, YP_{—}001371250.1, ZP_{—}07658682.1, YP_{—}002898825.1, ZP_{—}01547199.1, YP_{—}223070.1, ZP_{—}05161482.1, ZP_{—}04679742.1, YP_{—}002778618.1, ZP_{—}01626756.1, ZP_{—}05101564.1, YP_{—}002947374.1, NP_{—}385053.1, YP_{—}001328117.1, YP_{—}004493948.1, YP_{—}003339515.1, YP_{—}004699488.1, ZP_{—}05101969.1, YP_{—}485352.1, ZP_{—}01746033.1, ZP_{—}06712293.1, ZP_{—}01158125.1, ZP_{—}01058616.1, ZP_{—}05739755.1, NP_{—}949067.1, ZP_{—}02364657.1, YP_{—}570690.1, YP_{—}001208663.1, ZP_{—}02357557.1, ZP_{—}04751682.1, YP_{—}001326253.1, YP_{—}487666.1, ZP_{—}05167919.1, ADI18237.1, YP_{—}002825245.1, ZP_{—}02144858.1, ZP_{—}02188790.1, ZP_{—}06794586.1, YP_{—}001809828.1, YP_{—}997974.1, YP_{—}001476791.1, ZP_{—}08635286.1, YP_{—}676287.1, ZP_{—}07308228.1, ZP_{—}04596242.1, YP_{—}001622726.1, NP_{—}699590.1, ZP_{—}01446884.1, YP_{—}001168504.1, ZP_{—}01616388.1, ZP_{—}05117189.1, ZP_{—}05876432.1, ADT64694.1, ZP_{—}01754911.1, ZP_{—}05880498.1, ZP_{—}02360829.1, ZP_{—}06052433.1, ZP_{—}08663540.1, YP_{—}003768966.1, ZP_{—}02165422.1, ZP_{—}00960985.1, ZP_{—}07026655.1, YP_{—}001753039.1, YP_{—}371288.1, YP_{—}002974725.1, YP_{—}776880.1, ZP_{—}05784963.1, ZP_{—}05124380.1, YP_{—}459030.1, ZP_{—}05090690.1, ZP_{—}05064893.1, ZP_{—}02367982.1, ZP_{—}01890564.1, NP_{—}541848.1, ZP_{—}00960263.1, ZP_{—}02961617.1, YP_{—}001242097.1, ZP_{—}05838258.1,
in particular
Q00593.1, Q9WWW2.1, ZP_{—}00957061.1, YP_{—}957894.1, CAC38030.1, YP_{—}694430.1, YP_{—}957725.1, YP_{—}001672216.1, YP_{—}552061.1, YP_{—}130410.1, ZP_{—}06155535.1, ZP_{—}01222730.1, YP_{—}691907.1, YP_{—}002297804.1, YP_{—}004283522.1, YP_{—}001234383.1, YP_{—}004435031.1, ZP_{—}05110316.1, ZP_{—}05042898.1, YP_{—}004466324.1, ZP_{—}08553549.1, YP_{—}004125220.1, ADI22536.1, ADI18461.1, YP_{—}003810975.1, YP_{—}662346.1, YP_{—}004427557.1, YP_{—}692606.1, ZP_{—}05043291.1, YP_{—}440752.1, ZP_{—}02386160.1, ZP_{—}04763547.1, ZP_{—}02361232.1, YP_{—}003376674.1, ZP_{—}02354055.1, ZP_{—}05085930.1, ADQ00130.1, YP_{—}003643016.1, ZP_{—}05040520.1, YP_{—}691922.1, AAX23098.1, BAD07371.1, NP_{—}104379.1, YP_{—}002551960.1, YP_{—}003908558.1, YP_{—}987903.1, ZP_{—}05785860.1, YP_{—}004145612.1, YP_{—}004140926.1, CAZ88300.1,
and particularly preferably

### Q00593.1, Q9WWW2.1, ZP_{—}00957061.1, YP_{—}957894.1, CAC38030.1, YP_{—}694430.1, YP_{—}957725.1, YP_{—}001672216.1.

and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection is understood to mean in particular the conversion of ω-oxolauric acid and/or methyl ω-oxolaurate to ω-carboxylauric acid and/or methyl ω-carboxylaurate or the conversion of ω-hydroxylauric acid and/or methyl w-hydroxylaurate to ω-oxolauric acid and/or methyl ω-oxolaurate.

Such preferable alcohol dehydrogenases of EC 1.1.1.1 or EC 1.1.1.2 are selected from AdhE, AdhP, YjgB, YqhD, GIdA, EutG, YiaY, AdhE, AdhP, YhhX, YahK, HdhA, HisD, SerA, Tdh, Ugd, Udg, Gmd, YefA, YbiC, YdfG, YeaU, TtuC, YeiQ, YgbJ, YgcU, YgcT, YgcV, YggP, YgjR, YliI, YqiB, YzzH, LdhA, GapA, Epd, Dld, GatD, Gcd, GlpA, GlpB, GlpC, GlpD, GpsA and YphC from bacteria, in particular E. coli

and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection is understood to mean in particular the conversion of ω-oxolauric acid and/or methyl ω-oxolaurate to ω-carboxylauric acid and/or methyl ω-carboxylaurate or the conversion of ω-hydroxylauric acid and/or methyl ω-hydroxylaurate to ω-oxolauric acid and/or methyl ω-oxolaurate.

WO2010062480 A2 describes, particularly in practical examples 3, 4, 6 and 7, microorganisms which compared to their wild type are capable of forming more fatty acids and fatty acid derivatives, in particular fatty alcohols, from at least one simple carbon source. The document also describes enzymes F₄ preferred according to the invention and the sequences thereof in particular in FIG. 10 and practical examples 2 to 7.

### Enzyme E₅ ω-Oxidation 2

For the production of an (β-carboxy-functionalized carboxylic acid or an ω-carboxy-functionalized carboxylate ester it can be advantageous if the second genetic modification comprises increased activity of an enzyme E₅ which catalyses the conversion of ω-oxocarboxylic acids or ω-oxocarboxylate esters to the corresponding ω-carboxycarboxylic acids or ω-carboxycarboxylate esters.

### Specific Enzymes E₅

Preferably the enzyme E₅ is an aldehyde dehydrogenase of EC 1.2.1.3, EC 1.2.1.4 or EC 1.2.1.5, which preferentially catalyses the following reaction: ω-oxoalkanoic acid (ester)+NAD(P)⁺=ω-carboxyalkanoic acid (ester)+NAD(P)H⁺

Such preferable aldehyde dehydrogenases are selected from Prr, Usg, MhpF, AstD, GdhA, FrmA, Feab, Asd, Sad, PuuE, GabT, YgaW, BetB, PutA, PuuC, FeaB, AldA, Prr, EutA, GabD, AldB, TynA and YneI from bacteria, in particular E. coli

and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E₅ in general is understood to mean in particular the conversion of ω-oxolauric acid and/or methyl ω-oxolaurate to ω-carboxylauric acid and/or methyl ω-carboxylaurate.
alkL

In general, it can be advantageous for the microorganism according to the invention if it can secrete the ω-functionalized carboxylic acids and ω-functionalized carboxylate esters formed from the simple carbon source rapidly into the medium.

The organism advantageously achieves this in that the second genetic modification additionally comprises that the microorganism compared to its wild type forms more alkL gene product.

In connection with the present invention, the term “alkL gene product” is understood to mean proteins which fulfil at least one of the following two conditions:

1.) the protein is identified as a member of the superfamily of the OmpW proteins (protein family 3922 in the “Conserved Domain Database” (CDD) of the “National Center for Biotechnology Information” (NCBI)), where this assignment is made by alignment of the amino acid sequence of the protein with the database entries present in the CDD of the NCBI, which were deposited up to the 22.03.2010, with use of the standard search parameters, an e-value smaller than 0.01 and with use of the algorithm “blastp 2.2.23+”,
2.) in a search for conserved protein domains contained in the amino acid sequence concerned in the NCBI CDD (version 2.20) by means of RPS-BLAST, the presence of the conserved domain “OmpW, Outer membrane protein W” (COG3047) is identified with an e-value of less than 1×10⁻⁵ (“domain hit”).

Preferred gene products contained in the microorganism according to the invention alkL are characterized in that the production of the alkL gene product in the native host is induced by dicyclopropyl ketone; in this connection, it is also preferable that the expression of the alkL gene takes place as part of a group of genes, for example in a regulon such as for example an operon.

AlkL gene products contained in the microorganism according to the invention are preferably encoded by alkL genes from organisms selected from the group of the gram-negative bacteria, in particular the group containing, preferably consisting of, Pseudomonas sp., Azotobacter sp., Desulfitobacterium sp., Burkholderia sp., preferably Burkholderia cepacia, Xanthomonas sp., Rhodobacter sp., Ralstonia sp., Delftia sp. and Rickettsia sp., Oceanicaulis sp., Caulobacter sp., Marinobacter sp. and Rhodopseudomonas sp., preferably Pseudomonas putida, Oceanicaulis alexandrii, Marinobacter aquaeolei, in particular Pseudomonas putida GPo1 and P1, Oceanicaulis alexandrii HTCC2633, Caulobacter sp. K₃₁ and Marinobacter aquaeolei VT8. Quite especially preferred in this connection are alkL gene products encoded by the alkL genes from Pseudomonas putida GPo1 and P1, which are represented by Seq ID No. 1 and Seq ID No. 3, and proteins with polypeptide sequence Seq ID No. 2, Seq ID No. 4, Seq ID No. 5, Seq ID No. 6 or Seq ID No. 7 or with a polypeptide sequence wherein up to 60%, preferably up to 25%, particularly preferably up to 15%, in particular up to 10, 9, 8, 7, 6, 5, 4, 3, 2 or 1% of the amino acid residues are modified compared to Seq ID No. 2, Seq ID No. 4, Seq ID No. 5, Seq ID No. 6 or Seq ID No. 7 by deletion, insertion, substitution or a combination thereof and which still possess at least 50%, preferably 65%, particularly preferably 80%, in particular more than 90% of the activity of the protein with the respective reference sequence Seq ID No. 2, Seq ID No. 4, Seq ID No. 5, Seq ID No. 6 or Seq ID No. 7, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, this being in a system as described in the practical examples, wherein glucose is converted to ω-aminolauric acid in an E. coli cell. A method of choice for the determination of the synthesis level can be found in the practical examples.

For the definition of the units, the definition usual in enzyme kinetics applies here: 1 unit of biocatalyst converts 1 μmol of substrate to the product in one minute.

1 U=1 μmol/min.

### First Genetic Modification for Carboxylic Acid- and Carboxylate Ester-Synthesis

According to the invention, the microorganisms have a first genetic modification, so that compared to their wild type they are capable of forming more carboxylic acids and carboxylate ester from at least one simple carbon source.

In this connection, it is preferable according to the invention that the first genetic modification is, compared to the enzymatic activity of the wild type of the microorganism, increased activity of at least one of the enzymes selected from the group

Eᵢ Acyl-ACP (acyl carrier protein) thioesterase, preferably of EC 3.1.2.14 or EC 3.1.2.22, which catalyses the hydrolysis of an acyl-acyl carrier protein thioester,
Eᵢᵢ Acyl-CoA (coenzyme A) thioesterase, preferably of EC 3.1.2.2, EC 3.1.2.18, EC 3.1.2.19, EC 3.1.2.20 or EC 3.1.2.22, which catalyses the hydrolysis of an acyl-coenzyme A thioester,
E_{iib} Acyl-CoA (coenzyme A):ACP (acyl carrier protein) transacylase, which preferentially catalyses a reaction wherein a CoA thioester is converted into an ACP thioester,
Eᵢᵢᵢ Polyketide synthase, which catalyses a reaction which is also involved in the synthesis of carboxylic acids and carboxylate esters, and
Eᵢᵥ Hexanoic acid synthase, a specialized fatty acid synthase of the FAS-I type, which catalyses the synthesis of hexanoic acid from 2 molecules of malonyl-coenzyme A and one molecule of acetyl-coenzyme A.

### Specific Enzymes E₁

The reaction catalysed by Eᵢ differs from that catalysed by Eᵢᵢ only in that instead of an acyl-acyl carrier protein thioester an acyl-coenzyme A thioester is hydrolysed. It is obvious that because of significant side-activity many of the said enzymes Eᵢ can also be used as Eᵢᵢ and vice versa.

In cells preferred according to the invention, the enzyme Eᵢ is one which comprises sequences selected from:

AAC72881.1, ABB71579.1, CAC19934.1, AAC49180.1 (encoded by SEQ ID No. 10), AAC49783.1, AAC49179.1, CAB60830.1, ABB71581.1, AAC49269.1, CAC19933.1, CAA54060.1, AAC72882.1, Q39513.1, AAC49784.1, ABO38558.1, ABO38555.1, ABO38556.1, ABO38554.1, ADB79568.1, ADB79569.1, ACQ57188.1, ACQ57189.1, ABK96561.1, ACQ63293.1, ACQ57190.1, Q9SQI3.1, ABU96744.1, ABC47311.1, XP_{—}002324962.1, AAD01982.1, AAB51525.1, ACV40757.1, XP_{—}002309244.1, CBI28125.3, ABD91726.1, XP_{—}002284850.1, XP_{—}002309243.1, XP_{—}002515564.1, ACR56792.1, ACR56793.1, XP_{—}002892461.1, ABI18986.1, NP_{—}172327.1, CAA85387.1, CAA85388.1, ADA79524.1, ACR56795.1, ACR56794.1, CAN81819.1, ACF17654.1, AAB71729.1, ABH11710.1, ACQ57187.1, AAX51637.1, AAB88824.1, AAQ08202.1, AAB71731.1, AAX51636.1, CAC80370.1, CAC80371.1, AAG43858.1, ABD83939.1, AAD42220.2, AAG43860.1, AAG43861.1, AAG43857.1, AAL15645.1, AAB71730.1, NP_{—}001068400.1, EAY86877.1, NP_{—}001056776.1, XP_{—}002436457.1, NP_{—}001149963.1, ACN27901.1, EAY99617.1, ABL85052.1, XP_{—}002437226.1, NP_{—}001151366.1, ACF88154.1, NP_{—}001147887.1, XP_{—}002453522.1, BAJ99650.1, EAZ37535.1, EAZ01545.1, AAN17328.1, EAY86884.1, EEE57469.1, Q41635.1, AAM09524.1, Q39473.1, NP_{—}001057985.1, AAC49001.1, XP_{—}001752161.1, XP_{—}001770108.1, XP_{—}001784994.1, XP_{—}002318751.1, NP_{—}001047567.1, XP_{—}002322277.1, XP_{—}002299627.1, XP_{—}002511148.1, CBI15695.3, XP_{—}002299629.1, XP_{—}002280321.1, CAN60643.1, XP_{—}002459731.1, XP_{—}002975500.1, XP_{—}002962077.1, XP_{—}001773771.1, NP_{—}001151014.1, XP_{—}002317894.1, XP_{—}002971008.1, XP_{—}001774723.1, XP_{—}002280147.1, XP_{—}002526311.1, XP_{—}002517525.1, XP_{—}001764527.1, ABI20759.1, BAD73184.1, XP_{—}002987091.1, XP_{—}002985480.1, CBI26947.3, ABI20760.1, XP_{—}002303055.1, XP_{—}002885681.1, ADH03021.1, XP_{—}002532744.1, EAY74210.1, EEC84846.1, EEE54649.1, AAG35064.1, AAC49002.1, CAD32683.1, ACF78226.1, BAJ96402.1, XP_{—}002462626.1, NP_{—}001130099.1, XP_{—}002462625.1, ABX82799.3, Q42712.1, NP_{—}193041.1, AAB51524.1, NP_{—}189147.1, ABR18461.1, XP_{—}002863277.1, AAC72883.1, AAA33019.1, CBI40881.3, XP_{—}002262721.1, AAB51523.1, NP_{—}001063601.1, ADB79567.1, AAL77443.1, AAL77445.1, AAQ08223.1, AAL79361.1, CAA52070.1, AAA33020.1, CAA52069.1, XP_{—}001785304.1, CAC39106.1, XP_{—}002992591.1, XP_{—}002968049.1, XP_{—}001770737.1, XP_{—}001752563.1, AAG43859.1, XP_{—}002978911.1, XP_{—}002977790.1, ACB29661.1, XP_{—}002314829.1, XP_{—}002991471.1, EAZ45287.1, XP_{—}002986974.1, EEC73687.1, XP_{—}002312421.1, ACJ84621.1, NP_{—}001150707.1, AAD28187.1, XP_{—}001759159.1, XP_{—}001757193.1, XP_{—}002322077.1, ABE01139.1, XP_{—}002447294.1, AAX54515.1, AAD33870.1,
in particular
AAC72881.1, ABB71579.1, CAC19934.1, AAC49180.1 (encoded by SEQ ID No. 10), AAC49783.1, AAC49179.1, CAB60830.1, ABB71581.1, AAC49269.1, CAC19933.1, CAA54060.1, AAC72882.1, Q39513.1, AAC49784.1, AAC72883.1, Q41635.1, AAC49001.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme Eᵢ in general is understood to mean in particular the hydrolysis of dodecanoyl-ACP thioesters.

Microorganisms preferred according to the invention are those which are obtained when the microorganisms listed below having a first genetic modification in the sense of the invention are used as the starting point, in that they are provided with the second genetic modification and optionally at least one further genetic modification in the sense of the invention.

WO2010063031 A2 describes, particularly in paragraphs [0007] to [0008], [0092] to [0100], [0135] to [0136], [0181] to [0186] and [0204] to [0213] and practical examples 4 to 8, microorganisms preferably used according to the invention which have a first genetic modification, so that compared to their wild type they are capable of forming more microbial oil from at least one simple carbon source. The document also describes enzymes Eᵢ preferred according to the invention and the sequences thereof, particularly in paragraphs [0012] to [0013], [0155], [0160] to [0163], [0185] to [0190] and [0197] to [0199], FIG. 12, practical examples 4 to 8 and table 3.

WO2010063032 A2 describes, particularly in paragraphs [0007] to [0008], [0092] to [0100], to [0136], [0181] to [0186] and [0204] to [0213], and practical examples 4 to 8, microorganisms preferably used according to the invention which have a first genetic modification, so that compared to their wild type they are capable of forming more microbial oil from at least one simple carbon source. The document also describes enzymes Eᵢ preferred according to the invention and the sequences thereof, particularly in paragraphs [0012] to [0013], [0155], [0160] to [0163], [0185] to [0190] and [0197] to [0199], FIG. 12, practical examples 4 to 8 and table 3.

WO2011003034 A2 describes, particularly on page 3, second paragraph to page 7, first paragraph, page 20, second paragraph, to page 22, second paragraph, and on page 156 to page 166, fifth paragraph, and in claims 1 to 100, microorganisms preferably used according to the invention which have a first genetic modification, so that compared to their wild type they are capable of forming more fatty acids and fatty acid derivatives, in particular adipic acid, from at least one simple carbon source. The document also describes enzymes Eᵢ preferred according to the invention and the sequences thereof particularly on page 35, third paragraph, and page 36, first paragraph.

WO2011008565 A1 describes, particularly in paragraphs [0018] to [0024] and [0086] to [0102] and practical examples 2, 4, 7, 9 and 10, microorganisms preferably used according to the invention which have a first genetic modification, so that compared to their wild type they are capable of forming more fatty acids and fatty acid derivatives, in particular fatty acids, fatty aldehydes, fatty alcohols, alkanes and fatty acid ester, from at least one simple carbon source. The document also describes enzymes Eᵢ preferred according to the invention and the sequences thereof, particularly in paragraphs [0009] to [0018] and [0073] to [0082], FIGS. 1 to 3 and 7, table 4, practical examples 1 to 10 and claims 1 to 5 and 11 to 13.

WO2009076559 A1 describes, particularly in paragraphs [0013] to [0051] and [0064] to [00111] and claims 1 to 10, microorganisms preferably used according to the invention which have a first genetic modification, so that compared to their wild type they are capable of forming more fatty acids and fatty acid derivatives, in particular fatty acids, fatty alcohols, alkanes or alkenes, from at least one simple carbon source. The document also describes enzymes Eᵢ preferred according to the invention and the sequences thereof, particularly in table 1, paragraphs [0021], to [0030] and [0064] to [00111] and FIG. 6.

WO2010017245 A1 describes, particularly in paragraphs [0011] to [0015] and [00114] to [00134], practical example 3 and claims 1 to 2 and 9 to 11, microorganisms preferably used according to the invention which have a first genetic modification, so that compared to their wild type they are capable of forming more fatty acids and fatty acid derivatives from at least one simple carbon source. The document also describes enzymes Eᵢ preferred according to the invention and the sequences thereof, particularly in tables 1, 2 and 3, paragraphs [0080] to and claims 3 to 8.

WO2010127318 A2 describes, particularly on pages 1 to 9 and 11 to 16, practical examples 1, 2 and 4, FIGS. 1A to 1E and claims 23 to 43, 62 to 79 and 101 to 120, microorganisms preferably used according to the invention which have a first genetic modification, so that compared to their wild type they are capable of forming more fatty acids and fatty acid derivatives, in particular biodiesel equivalents and other fatty acid derivatives, above all fatty acid ethyl esters, fatty acid esters, wax esters, fatty alcohols and fatty aldehydes, from at least one simple carbon source. The document also describes enzymes Eᵢ preferred according to the invention and the sequences thereof, particularly on pages 17 and 19 to 23.

WO2008100251 A1 describes, particularly on pages 4 to 7 and 45 to 46, FIGS. 1A to 1E and claims 9 to 13, microorganisms preferably used according to the invention which have a first genetic modification, so that compared to their wild type they are capable of forming more fatty acids and fatty acid derivatives, in particular fatty acid esters, wax esters and fatty alcohols, from at least one simple carbon source. The document also describes enzymes Eᵢ preferred according to the invention and the sequences thereof, particularly on pages 4 to 5 and 45 to 46.

WO2007136762 A2 describes, particularly on pages 2 to 4 and 17 to 18, table 7, FIGS. 2 to 4, practical examples 2 to 8 and claims 13 and 35, microorganisms preferably used according to the invention which have a first genetic modification, so that compared to their wild type they are capable of forming more fatty acids and fatty acid derivatives, in particular fatty acid esters, wax esters, hydrocarbons and fatty alcohols, from at least one simple carbon source. The document also describes enzymes Eᵢ preferred according to the invention and the sequences thereof, particularly on pages 17 to 18, in tables 1, 7, 8 and 10 and FIG. 10.

WO2008113041 A2 describes, particularly on pages 35 to 41 and 64 to 67, FIG. 2, practical examples 6 and 10 and claims 7 and 36, microorganisms preferably used according to the invention which have a first genetic modification, so that compared to their wild type they are capable of forming more fatty acids and fatty acid derivatives, in particular fatty acid esters, wax esters, hydrocarbons, aliphatic ketones and fatty alcohols, from at least one simple carbon source. The document also describes enzymes Eᵢ preferred according to the invention and the sequences thereof, particularly in FIG. 7 and practical examples 6 and 10.

WO2010126891 A1 describes, particularly in paragraphs [0034] to [0091], [0195] to [0222] and to [0250], FIGS. 3 to 5 and practical examples 1 to 5, microorganisms preferably used according to the invention which have a first genetic modification, so that compared to their wild type they are capable of forming more fatty acids and fatty acid derivatives, in particular fatty acid esters, wax esters and fatty alcohols, from at least one simple carbon source. The document also describes enzymes Eᵢ preferred according to the invention and the sequences thereof, particularly in paragraphs [0245] to [0250], table 1 and practical examples 1 to 5.

WO2010118410 A1 describes, particularly in paragraphs [0022] to [0043], [0158] to [0197], FIGS. 1 to 4, practical examples 3 and 5 to 8 and claims 1 to 53 and 82 to 100, microorganisms preferably used according to the invention which have a first genetic modification, so that compared to their wild type they are capable of forming more fatty acids and fatty acid derivatives, in particular fatty acid esters and wax esters, from at least one simple carbon source. The document also describes enzymes Eᵢ preferred according to the invention and the sequences thereof, particularly in paragraphs [0158] to [0197], table 1, FIGS. 3 and 4 and practical examples 3 and 5 to 8.

WO2010118409 A1 describes, particularly in paragraphs [0134] to [0154], FIGS. 1 to 3 and 6 and practical example 3, microorganisms preferably used according to the invention which have a first genetic modification, so that compared to their wild type they are capable of forming more fatty acids and fatty acid derivatives, in particular fatty acid esters and wax esters, from at least one simple carbon source. The document also describes enzymes Eᵢ preferred according to the invention and the sequences thereof, particularly in paragraphs [0134] to [0154], FIGS. 3 and 6 and practical example 3.

WO2010075483 A2 describes, particularly in paragraphs [0061] to [0090], and [0287] to [0367], FIGS. 1, 4 and 5, practical examples 1 to 38 and claims 18 to 26, microorganisms preferably used according to the invention which have a first genetic modification, so that compared to their wild type they are capable of forming more fatty acids and fatty acid derivatives, in particular fatty acids, fatty acid methyl esters, fatty acid ethyl esters, fatty alcohols, fatty alkyl acetates, fatty aldehydes, fatty amines, fatty amides, fatty sulphates, fatty ethers, ketones, alkanes, internal and terminal olefins, dicarboxylic acids, α,ω-dicarboxylic acids and α,ω-diols, from at least one simple carbon source. The document also describes enzymes Eᵢ preferred according to the invention and the sequences thereof, particularly in paragraphs [0012] to [0060], tables 7, 17, 26 and 27, FIGS. 1, 44 to 47 and 55 to 59, practical examples 1 to 38 and claims 1 to 17.

WO2010062480 A2 describes, particularly in paragraphs [0022] to [0174] and [0296] to [0330], practical examples 3 and 5 to 8 and claims 17 and 24, microorganisms preferably used according to the invention which have a first genetic modification, so that compared to their wild type they are capable of forming more fatty acids and fatty acid derivatives, in particular fatty alcohols, from at least one simple carbon source. The document also describes enzymes Eᵢ preferred according to the invention and the sequences thereof, particularly in paragraphs [0022] to [0174], table 1, and practical examples 3 and 5 to 8.

WO2010042664 A2 describes, particularly in paragraphs [0022] to [0143] and [0241] to [0275], practical example 2 and claims 3 and 9, microorganisms preferably used according to the invention which have a first genetic modification, so that compared to their wild type they are capable of forming more fatty acids and fatty acid derivatives, in particular fatty aldehydes, from at least one simple carbon source. The document also describes enzymes Eᵢ preferred according to the invention and the sequences, particularly in table 1, FIG. 5 and practical example 2.

WO2011008535 A1 describes, particularly in paragraphs [0024] to [0032], and [0138] to [0158] and FIG. 13, microorganisms preferably used according to the invention which have a first genetic modification, so that compared to their wild type they are capable of forming more fatty acids and fatty acid derivatives, in particular carboxylic acids, hydroxycarboxylic acids and lactones thereof, from at least one simple carbon source.

WO2010022090 A1 describes, particularly in paragraphs [0022] to [0143] and [0238] to [0275], FIGS. 3 to 5, practical example 2 and claims 5, 15, 16 and 36, microorganisms preferably used according to the invention which have a first genetic modification, so that compared to their wild type they are capable of forming more fatty acids and fatty acid derivatives, in particular fatty acid esters and wax esters, from at least one simple carbon source. The document also describes enzymes Eᵢ preferred according to the invention and the sequences thereof, particularly in table 1, FIG. 6 and practical example 2.

WO2009140695 A1 describes, particularly in paragraphs [0214] to [0248] and practical examples 22 to 24, microorganisms preferably used according to the invention which have a first genetic modification, so that compared to their wild type they are capable of forming more fatty acids and fatty acid derivatives, in particular hydrocarbons, from at least one simple carbon source. The document also describes enzymes Eᵢ preferred according to the invention and the sequences thereof in particular in table 1, FIG. 40 and practical examples 22 to 24.

WO2010021711 A1 describes, particularly in paragraphs [0009] to [0020] and [0257] to [0317], FIGS. 3 to 5 and 19, practical examples 2 to 24 and claims 4, 5 and 30, microorganisms preferably used according to the invention which have a first genetic modification, so that compared to their wild type they are capable of forming more fatty acids and fatty acid derivatives, in particular fatty acid esters and wax esters, from at least one simple carbon source. The document also describes enzymes Eᵢ preferred according to the invention and the sequences thereof, in particular in table 3, FIG. 6 and practical examples 2 to 24.

WO2009085278 A1 describes, particularly in paragraphs [0188] to [0192] and FIG. 10, microorganisms preferably used according to the invention which have a first genetic modification, so that compared to their wild type they are capable of forming more fatty acids and fatty acid derivatives, in particular olefins, from at least one simple carbon source. The document also describes enzymes Eᵢ preferred according to the invention and the sequences thereof, particularly in table 1 and FIG. 10.

WO2011019858 A1 describes, particularly in paragraphs [0023], [0064] to [0074] and [0091] to [0099], practical examples 1 to 13, FIG. 1 and claim 8, microorganisms preferably used according to the invention which have a first genetic modification, so that compared to their wild type they are capable of forming more fatty acids and fatty acid derivatives, in particular fatty alcohols, from at least one simple carbon source. The document also describes preferred enzymes Eᵢ according to the invention and the sequences thereof, particularly in paragraphs [0085] to [0090], practical examples 1 to 13 and table 1.

WO2009009391 A2 describes, particularly in paragraphs [0010] to [0019] and [0191] to [0299], FIGS. 3 to 5, practical examples 2, 4 to 6, 9 to 14, 17 and 19 and claims 16, 39, 44 and 55 to 59, microorganisms preferably used according to the invention which have a first genetic modification, so that compared to their wild type they are capable of forming more fatty acids and fatty acid derivatives, in particular fatty acid esters, wax esters and fatty alcohols, from at least one simple carbon source. The document also describes enzymes Eᵢ preferred according to the invention and the sequences thereof, particularly in paragraphs [0010] to [0019] and [0191] to [0299], FIG. 9 and practical examples 2, 4 to 6, 9 to 14, 17 and 19.

WO2008151149 A2 describes, particularly in paragraphs [0009], [0015] to [0033], [0053], [0071], [0174] to [0191], [0274] and [0396], claims 53 to 114, 188 to 206 and 344 to 355 and tables 1 to 3, microorganisms preferably used according to the invention which have a first genetic modification, so that compared to their wild type they are capable of forming more microbial oil from at least one simple carbon source. The document also describes enzymes Eᵢ preferred according to the invention and the sequences thereof, particularly in table 5.

WO2008147781 A2 describes, particularly in paragraphs [0147] to [0156], practical examples 1 to 3, 8, 9 and 14 and claims 65 to 71, microorganisms preferably used according to the invention which have a first genetic modification, so that compared to their wild type they are capable of forming more fatty acids and fatty acid derivatives, in particular hydrocarbons, olefins and aliphatic ketones, from at least one simple carbon source. The document also describes enzymes Eᵢ preferred according to the invention and the sequences thereof in particular in practical examples 1 to 3, 8, 9 and 14.

WO2008119082 A2 describes, particularly on pages 3 to 5, 8 to 10 and 40 to 77, in FIGS. 4 and 5, practical examples 2 to 5 and 8 to 18 and claims 3 to 39 and 152 to 153, microorganisms preferably used according to the invention which have a first genetic modification, so that compared to their wild type they are capable of forming more fatty acids and fatty acid derivatives, in particular fatty acid esters, wax esters, triglycerides, biodiesel, gasoline, aviation fuel and fatty alcohols from at least one simple carbon source. The document also describes enzymes Eᵢ preferred according to the invention and the sequences thereof, particularly in table 1, FIG. 1, practical examples 2 to 5 and 8 to 18 and claims 124 to 134 and 138 to 141.

WO2010135624 A2 describes, particularly in paragraphs [0067] to [0083], and [0095] to [0098], microorganisms preferably used according to the invention which have a first genetic modification, so that compared to their wild type they are capable of forming more fatty acids and fatty acid derivatives, in particular fatty alcohols, from at least one simple carbon source. The document also describes enzymes Eᵢ preferred according to the invention and the sequences thereof, particularly in paragraphs [0067] to [0083] and [0095] to [0098]. Zheng Z, Gong Q, Liu T, Deng Y, Chen J C and Chen G Q. (Thioesterase II of Escherichia coli plays an important role in 3-hydroxydecanoic acid production. Appl Environ Microbiol. 2004. 70(7):3807-13) describe, particularly on pages 3808 to 3810 and 3012 and table 1, 3 and 4, microorganisms preferably used according to the invention which have a first genetic modification, so that compared to their wild type they are capable of forming more fatty acids and fatty acid derivatives, in particular fatty acid esters, wax esters and fatty alcohols, from at least one simple carbon source. The document also describes enzymes Eᵢ preferred according to the invention and the sequences thereof, particularly on pages 3807 and in table 2. Steen E J, Kang Y, Bokinsky G, Hu Z, Schirmer A, McClure A, Del Cardayre S B and Keasling J D (Microbial production of fatty acid-derived fuels and chemicals from plant biomass. Nature. 2010. 463(7280):559-62) describe, particularly on p. 559, third paragraph, to p. 559, first paragraph, microorganisms preferably used according to the invention which have a first genetic modification, so that compared to their wild type they are capable of forming more fatty acids and fatty acid derivatives, in particular fatty acids and fatty acid esters, from at least one simple carbon source. The document also describes enzymes Eᵢ preferred according to the invention and the sequences thereof, in particular in supplementary table 1.

Lennen R M, Braden D J, West R A, Dumesic J A and Pfleger B F (A process for microbial hydrocarbon synthesis: Overproduction of fatty acids in Escherichia coli and catalytic conversion to alkanes. Biotechnol Bioeng. 2010. 106(2):193-202) describe, particularly on p. 193, first paragraph, p. 194, first and second paragraph, p. 195, second paragraph to p. 197, second paragraph, p. 198, second paragraph to p. 199, third paragraph, microorganisms preferably used according to the invention which have a first genetic modification, so that compared to their wild type they are capable of forming more fatty acids and fatty acid derivatives, in particular fatty acids and fatty acid esters, from at least one simple carbon source. The document also describes enzymes Eᵢ preferred according to the invention and the sequences thereof, in particular on p. 193, first paragraph, p. 194, first and second paragraph, p. 196, second paragraph, and in the supplementary material.

Liu T, Vora H and Khosla C. (Quantitative analysis and engineering of fatty acid biosynthesis in E. coli. Metab Eng. 2010 July; 12(4):378-86.) describe, particularly in sections 2.2, and 3.1 and in table 1 and 2, microorganisms preferably used according to the invention which have a first genetic modification, so that compared to their wild type they are capable of forming more fatty acids and fatty acid derivatives, in particular fatty acids and fatty acid esters, from at least one simple carbon source. The document also describes enzymes E₁ preferred according to the invention and the sequences thereof, in particular in table 1.

Yuan L, Voelker T A and Hawkins D J. (Modification of the substrate specificity of an acyl-acyl carrier protein thioesterase by protein engineering. Proc Natl Acad Sci USA. 1995 Nov. 7; 92(23):10639-43) describe, particularly on p. 10641, fourth paragraph, and in FIG. 2 and table 1, microorganisms preferably used according to the invention which have a first genetic modification, so that compared to their wild type they are capable of forming more fatty acids and fatty acid derivatives, in particular fatty acids and fatty acid esters, from at least one simple carbon source. The document also describes enzymes Eᵢ preferred according to the invention and the sequences thereof, in particular on p. 10639 first paragraph, p. 10640, second, third and last paragraph, p. 10641, second and third paragraph, and in FIG. 1 and table 1 and 2.

Liu X, Vora H and Khosla C. (Overproduction of free fatty acids in E. coli: implications for biodiesel production. Metab Eng. 2008. 10(6):333-9.) describe, particularly on p. 334, second paragraph, paragraphs 2.2, 2.3 and 3 (first to fourth paragraph) and in table 1, microorganisms preferably used according to the invention which have a first genetic modification, so that compared to their wild type they are capable of forming more fatty acids and fatty acid derivatives, in particular fatty acids and fatty acid esters, from at least one simple carbon source. The document also describes enzymes Eᵢ preferred according to the invention and the sequences thereof, in particular in paragraph 2.2.

Liu X, Sheng J and Curtiss IIII R. (Fatty acid production in genetically modified cyanobacteria. Proc Natl Acad Sci USA. 2011. 108(17):6899-904) describe, particularly on p. 6899, fourth and last paragraph, p. 6900, first to penultimate paragraph, and in table S1 of the “Supporting Information”, microorganisms preferably used according to the invention which have a first genetic modification, so that compared to their wild type they are capable of forming more fatty acids and fatty acid derivatives, in particular fatty acids and fatty acid esters, from at least one simple carbon source. The document also describes enzymes Eᵢ preferred according to the invention and the sequences thereof, in particular on p. 6899, sixth and last paragraph.

### Specific Enzymes Eᵢᵢ

Microorganisms preferred according to the invention are those which are obtained when the microorganisms listed below having a first genetic modification in the sense of the invention are used as the starting point, in that they are provided with the second genetic modification and optionally at least one further genetic modification in the sense of the invention.

Steen E J, Kang Y, Bokinsky G, Hu Z, Schirmer A, McClure A, Del Cardayre S B and Keasling J D (Microbial production of fatty-acid-derived fuels and chemicals from plant biomass. Nature. 2010. 463(7280):559-62) describe, particularly on p. 559, third paragraph, to p. 559, first paragraph, microorganisms preferably used according to the invention which have a first genetic modification, so that compared to their wild type they are capable of forming more carboxylic acids and carboxylate esters, in particular fatty acids and fatty acid esters, from at least one simple carbon source. The document also describes enzymes Eᵢᵢ preferred according to the invention and the sequences thereof, in particular in supplementary table 1.

Lennen R M, Braden D J, West R A, Dumesic J A and Pfleger B F (A process for microbial hydrocarbon synthesis: Overproduction of fatty acids in Escherichia coli and catalytic conversion to alkanes. Biotechnol Bioeng. 2010. 106(2):193-202) describe, particularly on p. 193, first paragraph, p. 194, first and second paragraph, p. 195, second paragraph to p. 197, second paragraph, p. 198, second paragraph to p. 199, third paragraph, microorganisms preferably used according to the invention which have a first genetic modification, so that compared to their wild type they are capable of forming more carboxylic acids and carboxylate esters, in particular fatty acids and fatty acid esters, from at least one simple carbon source. The document also describes enzymes Eᵢᵢ preferred according to the invention and the sequences thereof, in particular on p. 193, first paragraph, p. 194, first and second paragraph, p. 196, second paragraph, and in the supplementary material.

Liu T, Vora H and Khosla C. (Quantitative analysis and engineering of fatty acid biosynthesis in E. coli. Metab Eng. 2010 July; 12(4):378-86.) describe, particularly in sections 2.2, and 3.1 and in table 1 and 2, microorganisms preferably used according to the invention which have a first genetic modification, so that compared to their wild type they are capable of forming more carboxylic acids and carboxylate esters, in particular fatty acids and fatty acid esters, from at least one simple carbon source. The document also describes enzymes Eᵢᵢ preferred according to the invention and the sequences thereof, in particular in table 1.

Yuan L, Voelker T A and Hawkins D J. (Modification of the substrate specificity of an acyl-acyl carrier protein thioesterase by protein engineering. Proc Natl Acad Sci USA. 1995 Nov. 7; 92(23):10639-43) describe, particularly on p. 10641, fourth paragraph, and in FIG. 2 and table 1 microorganisms preferably used according to the invention which have a first genetic modification, so that compared to their wild type they are capable of forming more carboxylic acids and carboxylate esters, in particular fatty acids and fatty acid esters, from at least one simple carbon source. The document also describes enzymes Eᵢᵢ preferred according to the invention and the sequences thereof, in particular on p. 10639 first paragraph, p. 10640, second, third and last paragraph, p. 10641, second and third paragraph, and in FIG. 1 and table 1 and 2.

Liu X, Vora H and Khosla C. (Overproduction of free fatty acids in E. coli: implications for biodiesel production. Metab Eng. 2008. 10(6):333-9.) describe, particularly on p. 334, second paragraph, paragraphs 2.2, 2.3 and 3 (first to fourth paragraph) and in table 1, microorganisms preferably used according to the invention which have a first genetic modification, so that compared to their wild type they are capable of forming more carboxylic acids and carboxylate esters, in particular fatty acids and fatty acid esters, from at least one simple carbon source. The document also describes enzymes E_{d} preferred according to the invention and the sequences thereof, in particular in paragraph 2.2.

Liu X, Sheng J and Curtiss IIII R. (Fatty acid production in genetically modified cyanobacteria. Proc Natl Acad Sci USA. 2011. 108(17):6899-904) describe, particularly on p. 6899, fourth and last paragraph, p. 6900, first to penultimate paragraph, and in table S1 of the “Supporting Information”, microorganisms preferably used according to the invention which have a first genetic modification, so that compared to their wild type they are capable of forming more carboxylic acids and carboxylate esters, in particular fatty acids and fatty acid esters, from at least one simple carbon source. The document also describes enzymes Eᵢ preferred according to the invention and the sequences thereof, in particular on p. 6899, sixth and last paragraph.

### Specific Enzymes Eᵢᵢᵢ

Microorganisms preferred according to the invention are those which are obtained when the microorganisms listed below having a first genetic modification in the sense of the invention are used as the starting point, in that they are provided with the second genetic modification and optionally at least one further genetic modification in the sense of the invention.

WO2009121066 A1 describes, particularly in claims 8 to 14, microorganisms preferably used according to the invention which have a first genetic modification, so that compared to their wild type they are capable of forming more fatty acids and fatty acid derivatives, in particular dicarboxylic acids, from at least one simple carbon source. The document also describes enzymes Eᵢᵢᵢ preferred according to the invention and the sequences thereof, particularly in paragraphs [00026] to [0054], practical examples 1 to 6, FIGS. 4 to 10 and claims 1 to 7.

WO2009134899 A1 describes, particularly in paragraphs [0079] to [0082], practical example 1 and claim 20, microorganisms preferably used according to the invention which have a first genetic modification, so that compared to their wild type they are capable of forming more fatty acids and fatty acid derivatives, in particular carboxylic acids, hydroxycarboxylic acids and lactones thereof, from at least one simple carbon source. The document also describes enzymes Eᵢᵢᵢ preferred according to the invention and the sequences thereof, particularly in paragraphs [0009] to [0010] and [0044] to [0078], practical example 1, FIGS. 1 and 5 to 8 and claims 15 to 17 and 19.

### Specific Enzymes Eᵢᵥ

In cells preferred according to the invention, the enzyme is one which comprises sequences selected from:

AAS90071.1, XP_{—}002379948.1, AAS90024.1, XP_{—}001821514.2, BAE59512.1, AAL99898.1, AAS90001.1, AAS90049.1, XP_{—}001911518.1, ACH72901.1, XP_{—}681084.1, AAC49198.1, EFW18013.1, XP_{—}003070494.1, XP_{—}001241401.1, XP_{—}002384449.1, XP_{—}001827206.1, XP_{—}002836001.1, XP_{—}001393196.1, XP_{—}660984.1, XP_{—}001395284.1, XP_{—}002148677.1, XP_{—}001827151.2, BAE66018.1, XP_{—}001217254.1, CAK40139.1, XP_{—}001393516.2, XP_{—}002477829.1, XP_{—}002146311.1, XP_{—}002340042.1, XP_{—}002544942.1, CBF87553.1, XP_{—}002149766.1, 2UV8_A, XP_{—}682676.1, CBX98966.1, XP_{—}002560069.1, XP_{—}001273102.1, P15368.1, XP_{—}001273530.1, CBX99714.1, AAB41493.1, XP_{—}001823764.1, XP_{—}001388458.1, XP_{—}748738.1, EDP53207.1, XP_{—}001259179.1, XP_{—}001825741.2, BAE64608.1, XP_{—}001213437.1, XP_{—}002377327.1, XP_{—}002152724.1, EFZ04065.1, XP_{—}001792784.1, EGP89632.1, XP_{—}001407660.1, EFQ31023.1, XP_{—}003040066.1, 2UV9_A, XP_{—}002486436.1, XP_{—}001585982.1, EFY87204.1, XP_{—}002620504.1, XP_{—}003295647.1, EEQ86108.1, XP_{—}001938586.1, XP_{—}001547465.1, XP_{—}001906653.1, XP_{—}001402457.2, CAK40502.1, XP_{—}002568116.1, XP_{—}003230922.1, XP_{—}001647236.1, XP_{—}385497.1, EGD94294.1, EGE05134.1, XP_{—}002849847.1, XP_{—}003015737.1, EFX06093.1, XP_{—}003019052.1, EEH03423.1, XP_{—}001942351.1, EGC45478.1, XP_{—}002556020.1, XP_{—}003011025.1, CAY86729.1, EDN60916.1, EGA84463.1, EGA56454.1, EEU05652.1, NP_{—}015093.1, XP_{—}003231214.1, XP_{—}445956.1, EGA60201.1, XP_{—}003349949.1, XP_{—}003070417.1, XP_{—}001241314.1, EGR48038.1, XP_{—}002615278.1, EFW15042.1, EGO59647.1, XP_{—}452914.1, XP_{—}962466.1, XP_{—}001537327.1, XP_{—}002796517.1, XP_{—}003305240.1, XP_{—}002543037.1, XP_{—}002499262.1, NP_{—}985412.2, XP_{—}003019770.1, EFW96269.1, XP_{—}002843350.1, EEH43965.1, XP_{—}457388.1, XP_{—}001799391.1, EEH21370.1, BAD08376.1, XP_{—}001486434.1, BAF79876.1, EFY90998.1, XP_{—}001939431.1, EER44845.1, EFZ02060.1, XP_{—}001386834.2, XP_{—}501096.1, XP_{—}003299758.1, XP_{—}002419391.1, XP_{—}002490414.1, ACZ66251.1, XP_{—}002548204.1, P43098.1, XP_{—}002176039.1, XP_{—}002479407.1, EEQ44526.1, AAA34601.1, XP_{—}001791764.1, XP_{—}003009337.1, BAA11913.1, NP_{—}593823.1, BAB62031.1, BAB62032.1, BAB62030.1, 2 PFF_A, XP_{—}380212.1, ADN94478.1, EGF83443.1, XP_{—}681149.1, EGG00662.1, ADN94479.1, ABC94883.1, XP_{—}571099.1, EFY94095.1, EFW39589.1, XP_{—}003194430.1, XP_{—}003031600.1, XP_{—}001836417.1, XP_{—}001880844.1, XP_{—}762607.1, EGN98830.1, EGO24420.1, ACD87451.1, XP_{—}003328630.1, XP_{—}002997955.1, CCA25392.1, XP_{—}002901724.1, EFY86381.1, XP_{—}002901728.1, ADN97213.1, XP_{—}759118.1, XP_{—}003325251.1, XP_{—}003169619.1, XP_{—}002555446.1, ABJ98780.1, XP_{—}723161.1, EDZ68993.1, XP_{—}001526334.1, XP_{—}001223165.1, YP_{—}889015.1, AAO43178.1, YP_{—}001702252.1, XP_{—}003026305.1, YP_{—}003659808.1, ZP_{—}08155637.1, ZP_{—}04749666.1, ZP_{—}08022190.1, YP_{—}004007770.1, YP_{—}954908.1, YP_{—}004522637.1, YP_{—}640811.1, ZP_{—}04448562.1, NP_{—}301868.1, ZP_{—}06851996.1, YP_{—}003273140.1, YP_{—}001071929.1, YP_{—}001133797.1, YP_{—}004076455.1, YP_{—}701403.1, ZP_{—}03324816.1, YP_{—}002778327.1, ZP_{—}02028077.1, YP_{—}909119.1, YP_{—}880884.1, YP_{—}002767320.1, NP_{—}961266.1, ZP_{—}07457010.1, ZP_{—}08206945.1, ZP_{—}02917151.1, ZP_{—}04387794.1, YP_{—}003359863.1, EGO39886.1, ABE96385.1, ZP_{—}05228143.1, ZP_{—}06522069.1, EGL13180.1, ZP_{—}06976698.1, YP_{—}001852225.1, ZP_{—}06596502.1, YP_{—}907384.1, ZP_{—}06518033.1, AEF27803.1, YP_{—}003374392.1, ZP_{—}07485570.1, NP_{—}217040.1, ZP_{—}03742148.1, NP_{—}856198.1, YP_{—}004724192.1, NP_{—}337093.1, AEJ51135.1, ZP_{—}05765008.1, YP_{—}004745991.1, AEJ47516.1, ZP_{—}06927266.1, ZP_{—}03646962.1, AEF31807.1, YP_{—}003939358.1, YP_{—}003971698.1, YP_{—}003986333.1, ZP_{—}05750911.1, ADD61451.1, ZP_{—}07942485.1, YP_{—}004209716.1, YP_{—}004221489.1, AEI96705.1, NP_{—}696693.1, AEG82252.1, YP_{—}004001156.1, ZP_{—}03976473.1, ZP_{—}04663991.1, ZP_{—}00121397.1, YP_{—}003662064.1, YP_{—}003646283.1, YP_{—}004630447.1, YP_{—}002323720.1, YP_{—}002835610.1, YP_{—}117466.1, ZP_{—}02963252.1, ADC85342.1, NP_{—}940183.1, NP_{—}739002.1, ZP_{—}06755645.1, ADL21513.1, YP_{—}003784047.1, ADL11108.1, ZP_{—}06608499.1, ZP_{—}07967121.1, ZP_{—}05966223.1, ZP_{—}08682531.1, ZP_{—}03918327.1, ZP_{—}07879655.1, ZP_{—}03972703.1, ZP_{—}06162645.1, ZP_{—}06837277.1, ZP_{—}07990916.1, ZP_{—}03394081.1, CAA46024.1, YP_{—}004760934.1, ZP_{—}06751771.1, ZP_{—}03934033.1, NP_{—}601696.1, BAB99888.1, YP_{—}001139316.1, ZP_{—}03926457.1, NP_{—}737523.1, ZP_{—}02044858.1, ZP_{—}07404023.1, ZP_{—}03709883.1, XP_{—}002388648.1, ZP_{—}07402466.1, ZP_{—}03710807.1, ZP_{—}08294093.1, ZP_{—}08232611.1, XP_{—}682514.1, ZP_{—}06837028.1, YP_{—}001137826.1, CAA61087.1, ZP_{—}06043461.1, YP_{—}002833817.1, YP_{—}225128.1, NP_{—}600065.1, ABU23831.1, ZP_{—}07716892.1, ZP_{—}03935133.1, ZP_{—}02549600.1, ZP_{—}05215994.1, YP_{—}004494858.1, XP_{—}001526333.1, AAS90085.1, XP_{—}002379947.1, AAS90025.1, XP_{—}001821515.1, AAL99899.1, AAS90002.1, AAS90050.1, XP_{—}001911517.1, ACH72900.1, XP_{—}681083.1, AAC49199.1, XP_{—}003070495.1, XP_{—}001241402.1, EFW18012.1, CBX98970.1, EEH03422.1, EEQ86107.1, EGC45479.1, XP_{—}002620503.1, XP_{—}001537328.1, XP_{—}002796516.1, 2UVA_G, EEH43966.1, DAA05950.1, EGR47893.1, XP_{—}003070418.1, XP_{—}001241316.1, XP_{—}001827193.1, XP_{—}002384436.1, XP_{—}682677.1, XP_{—}002486435.1, EGP88608.1, EDP53206.1, XP_{—}001259180.1, EEH21369.1, XP_{—}002543038.1, XP_{—}748739.1, XP_{—}003015735.1, EGE05135.1, XP_{—}002152723.1, XP_{—}002560068.1, XP_{—}001273529.1, XP_{—}003230923.1, EFX05327.1, XP_{—}003019051.1, XP_{—}001585981.1, XP_{—}361644.2, XP_{—}001223166.1, XP_{—}003349948.1, XP_{—}002380737.1, AAB41494.1, XP_{—}001823765.1, XP_{—}962465.1, EGO59648.1, XP_{—}001906652.1, XP_{—}003039864.1, XP_{—}001213436.1, XP_{—}385498.1, XP_{—}003295646.1, EFQ31022.1, XP_{—}002849848.1, XP_{—}002148679.1, CBX99715.1, XP_{—}002149767.1, EFY87205.1, EFZ04064.1, XP_{—}002340041.1, EGD94295.1, XP_{—}001938587.1, CAK45758.1, XP_{—}001792785.1, XP_{—}001393189.2, XP_{—}003169620.1, XP_{—}001547461.1, XP_{—}001217253.1, XP_{—}001939430.1, BAA92930.1, Q92215.1, EDK38075.2, EFW97345.1, XP_{—}002495511.1, XP_{—}451653.1, XP_{—}500912.1, CAA42211.1, XP_{—}001486502.1, XP_{—}002477835.1, XP_{—}445436.1, NP_{—}594370.1, XP_{—}001827152.2, BAE66019.1, BAA36384.1, BAB62141.1, XP_{—}003299759.1, XP_{—}002553365.1, XP_{—}002489642.1, 2UV8_G, XP_{—}457311.1, CAY80909.1, XP_{—}001395285.1, EGA61562.1, EDN60099.1, EDV12927.1, NP_{—}012739.1, XP_{—}002616181.1, XP_{—}002420328.1, XP_{—}001524822.1, XP_{—}002550943.1, XP_{—}001386364.2, NP_{—}984945.2, 227846, AAB59310.1, XP_{—}001646561.1, XP_{—}716877.1, XP_{—}001836417.1, XP_{—}002146312.1, P34731.1, EGO24420.1, XP_{—}002544941.1, EFZ02054.1, XP_{—}002175228.1, XP_{—}001393490.2, XP_{—}003031600.1, XP_{—}002479408.1, XP_{—}002568119.1, XP_{—}001825735.2, XP_{—}002377320.1, EGN98830.1, ACD87451.1, XP_{—}001880844.1, XP_{—}571100.1, ABC94882.1, XP_{—}775164.1, BAE64602.1, EFY90992.1, XP_{—}003194424.1, XP_{—}001273103.1, XP_{—}681142.1, XP_{—}003011020.1, AAA34602.1, XP_{—}003231209.1, XP_{—}003019765.1, ADN94478.1, EEQ46070.1, XP_{—}001799393.1, CAK40504.1, AAM75418.1, ADN94479.1, XP_{—}002843356.1, CAA27616.1, XP_{—}380213.1, ADN97213.1, XP_{—}759118.1, XP_{—}762607.1, CAK49094.1, EER44843.1, XP_{—}003009335.1, XP_{—}002997955.1, XP_{—}002901724.1, CCA25392.1, CAK36856.1, XP_{—}001388457.2, ABO37974.1, ABJ98780.1, XP_{—}660985.1, EDZ71063.1, XP_{—}001402459.2, XP_{—}001791765.1, XP_{—}003324647.1, EGG10429.1, EFW15039.1, XP_{—}002384390.1, XP_{—}003031976.1, EDZ71062.1, EFW39589.1, ACZ80683.1, XP_{—}002901728.1, XP_{—}003328630.1, XP_{—}681125.1, XP_{—}003325251.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme Eᵢᵥ in general is understood to mean in particular the conversion to hexanoic acid from 2 molecules of malonyl-coenzyme A and one molecule of acetyl-coenzyme A.

Microorganisms preferred according to the invention are those which are obtained when the microorganisms listed below having a first genetic modification in the sense of the invention are used as the starting point, in that they are provided with the second genetic modification and optionally at least one further genetic modification in the sense of the invention.

WO2011003034 A2 describes, particularly on p. 2 to 3, p. 5 third paragraph, in practical examples 1 to 4, 7 to 9 and 12 to 14 and claims 1 to 100, microorganisms preferably used according to the invention which have a first genetic modification, so that compared to their wild type they are capable of forming more fatty acids and fatty acid derivatives, in particular hexanoic acid, from at least one simple carbon source. The document also describes enzymes Eᵢᵥ preferred according to the invention and the sequences thereof, particularly on p. 5 and in practical example 3.

Hitchman T S, Schmidt E W, Trail F, Rarick M D, Linz J E and Townsend C A. (Hexanoate synthase, a specialized type I fatty acid synthase in aflatoxin B1 biosynthesis. Bioorg Chem. 2001. 29(5):293-307) describe, particularly on p. 296, penultimate paragraph, to p. 298, second paragraph, microorganisms preferably used according to the invention which have a first genetic modification, so that compared to their wild type they are capable of forming more fatty acids and fatty acid derivatives, in particular hexanoic acid, from at least one simple carbon source. The document also describes enzymes Eᵢᵥ preferred according to the invention and the sequences thereof in in particular p. 299, fourth paragraph, to p. 302, first paragraph.

In connection with the first genetic modification, it can be beneficial instead of the enzyme Eᵢ to use a combination of increasing the activity of an enzyme Eᵢᵢ compared to that of the wild type paired with that of an enzyme E_{iib} which catalyses a reaction wherein a CoA thioester is converted into an ACP thioester.

Suitable enzymes E_{iib} are known as acyl-CoA (coenzyme A):ACP (acyl carrier protein) transacylases. Preferred enzymes E_{iib} are selected from

XP_{—}003402554.1, YP_{—}002908243.1, YP_{—}001778804.1, YP_{—}001670627.1, YP_{—}004703658.1, YP_{—}001747923.1, YP_{—}004348703.1, YP_{—}004352505.1, YP_{—}004379169.1, ADR61731.1, YP_{—}001269622.1, YP_{—}001186851.1, YP_{—}004659609.1, YP_{—}003519049.1, YP_{—}001811696.1, YP_{—}004616040.1, NP_{—}252697.1, NP_{—}252169.1, NP_{—}249421.1, ZP_{—}06456665.1, ZP_{—}01167071.1, ZP_{—}08557569.1, ZP_{—}08554397.1, YP_{—}001157914.1, YP_{—}004475334.1, EGM20156.1, BAK10182.1, YP_{—}347066.1, Q9KJH8.1, YP_{—}002987902.1, ZP_{—}03794633.1, ZP_{—}03627777.1, YP_{—}004434330.1, NP_{—}743567.1, ZP_{—}03456835.1, ZP_{—}07911512.1, ZP_{—}07264431.1, ZP_{—}02265387.2, ZP_{—}03456013.1, ZP_{—}07577798.1, ZP_{—}08429367.1, YP_{—}004055319.1, YP_{—}004053883.1, YP_{—}275219.1, YP_{—}276116.1, YP_{—}003882762.1, EGH97259.1, EGH95622.1, EGH90852.1, EGH85976.1, EGH81248.1, EGH79586.1, EGH79549.1, EGH73565.1, EGH66549.1, EGH64812.1, EGH58099.1, EGH54896.1, EGH50352.1, EGH43364.1, EGH41593.1, EGH29888.1, EGH29417.1, EGH22392.1, EGH22129.1, EGH11618.1, EGH10011.1, ZP_{—}04589662.1, CCA60711.1, YP_{—}003004716.1, BAK16630.1, YP_{—}003264146.1, YP_{—}371314.1, YP_{—}439272.1, NP_{—}762892.1, ADW02533.1, YP_{—}003291774.1, EGC99875.1, ZP_{—}08139631.1, YP_{—}003333890.1, EGC08366.1, YP_{—}080427.1, YP_{—}258557.1, YP_{—}001985016.1, YP_{—}002875182.1, YP_{—}002871082.1, YP_{—}237050.1, YP_{—}236199.1, NP_{—}794008.1, NP_{—}793082.1, YP_{—}609790.1, EFW81598.1, EFW79804.1, ZP_{—}07261632.1, ZP_{—}07229875.1, ZP_{—}06458504.1, ZP_{—}05640568.1, ZP_{—}03399268.1, ZP_{—}03398232.1, ZP_{—}08004496.1, ZP_{—}06876938.1, ZP_{—}03227482.1, ZP_{—}02511781.1, ZP_{—}02503964.1, ZP_{—}02477255.1, ZP_{—}02466678.1, ZP_{—}02465791.1, ZP_{—}02461688.1, ZP_{—}02417235.1, ZP_{—}02414413.1, ZP_{—}02408727.1, ZP_{—}02376540.1, ZP_{—}02358949.1, ZP_{—}07778021.1, ZP_{—}07774051.1, ZP_{—}07795409.1, ZP_{—}07089008.1, YP_{—}776393.1, ZP_{—}07684652.1, ZP_{—}06640022.1, ZP_{—}03054335.1, ZP_{—}02907621.1, ZP_{—}02891475.1, ZP_{—}01862226.1, ZP_{—}01769192.1, ZP_{—}01367441.1, ZP_{—}01366930.1, ZP_{—}01364106.1, ZP_{—}01312991.1, ZP_{—}01173135.1, ZP_{—}07005523.1, ZP_{—}04955702.1, ZP_{—}04943305.1, ZP_{—}04936014.1, ZP_{—}04932415.1, ZP_{—}04930223.1, ZP_{—}04905334.1, ZP_{—}04893870.1, ZP_{—}04893165.1, ZP_{—}04892059.1, ZP_{—}04884056.1, YP_{—}002438575.1, YP_{—}002234939.1, YP_{—}001488024.1, YP_{—}001346487.1, YP_{—}001350135.1, YP_{—}001347031.1, YP_{—}990329.1, YP_{—}860279.1, YP_{—}789111.1, YP_{—}792557.1, YP_{—}623139.1, YP_{—}175644.1, YP_{—}111362.1, YP_{—}110557.1, YP_{—}105231.1, NP_{—}937516.1, AAU44816.1, AAA25978.1, XP_{—}002721010.1, AAK81868.1, AAK71350.1, AAK71349.1, ZP_{—}06499968.1, ZP_{—}06498781.1, YP_{—}003472045.1, ACA03779.1, ABL84756.1, AAQ16175.1, AAT51302.1, AAT51199.1, ZP_{—}05639386.1, ACH70299.1, ACA60824.1, BAB32432.1,
in particular
AAK81868.1, NP_{—}743567.1, AAK71349.1, YP_{—}001269622.1, ADR61731.1, AAU44816.1, AAQ16175.1, YP_{—}001670627.1, ACH70299.1, Q9KJH8.1, YP_{—}004703658.1, ZP_{—}08139631.1, YP_{—}609790.1, YP_{—}001747923.1, YP_{—}258557.1, YP_{—}347066.1, YP_{—}002871082.1, YP_{—}004352505.1, ACA60824.1, ZP_{—}07774051.1, BAB32432.1, ZP_{—}05640568.1, EGH58099.1, EGH64812.1, EGH11618.1, ZP_{—}06456665.1, YP_{—}276116.1, EFW81598.1, EGH95622.1, EGH22129.1, NP_{—}794008.1, ZP_{—}03399268.1, ZP_{—}07264431.1, EGH73565.1, YP_{—}237050.1, ZP_{—}06498781.1, EGH29888.1, EGH79586.1, EGH50352.1, YP_{—}792557.1, YP_{—}001350135.1, ZP_{—}01364106.1, ZP_{—}04932415.1, NP_{—}249421.1, YP_{—}004379169.1, ACAO3779.1, YP_{—}001186851.1, YP_{—}004475334.1, ZP_{—}04589662.1, ZP_{—}03398232.1, EGH10011.1, ZP_{—}07229875.1, ZP_{—}05639386.1, EGH66549.1, YP_{—}275219.1, ZP_{—}07005523.1, EFW79804.1, ZP_{—}06458504.1, EGH85976.1, YP_{—}236199.1, EGH43364.1, ZP_{—}07261632.1, ZP_{—}06499968.1, EGH29417.1, EGH54896.1, EGH22392.1, EGH97259.1, NP_{—}793082.1, EGH90852.1, EGH41593.1, NP_{—}252169.1, ZP_{—}01366930.1, YP_{—}001347031.1, ZP_{—}07778021.1, YP_{—}002875182.1, AAA25978.1, ABL84756.1, EGH81248.1, ZP_{—}07795409.1
and particularly preferably AAU44816.1, NP_{—}743567.1, YP_{—}001269622.1, ADR61731.1, AAK71349.1, YP_{—}001670627.1, AAK81868.1, AAQ16175.1, Q9KJH8.1, ACH70299.1, YP_{—}004703658.1, ZP_{—}08139631.1, YP_{—}609790.1, YP_{—}001747923.1, AAK71350.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E_{iib} in general is understood to mean in particular the conversion of dodecanoyl-CoA thioester to dodecanoyl-ACP thioester.

### Third Genetic Modification for the Production of Carboxylate Esters

In particular for the production of ω-functionalized carboxylate esters, such as for example ω-hydroxy-, ω-carboxy- or ω-aminocarboxylate esters it is advantageous if the microorganism has a third genetic modification, which compared with the enzymatic activity of the wild type of the microorganism comprises increased activity of at least one of the enzymes E_{iib}, Eᵥ, Eᵥᵢ, or Eᵥᵢᵢ which are involved in the conversion of carboxylic acids or ω-functionalized carboxylic acids to carboxylate esters or ω-functionalized carboxylate esters.

In this connection, it is preferable according to the invention that this genetic modification is, compared to the enzymatic activity of the wild type of the microorganism, increased activity of at least one of the enzymes selected from the group

E_{iib} Acyl-CoA (coenzyme A):ACP (acyl carrier protein) transacylase, which converts an ACP thioester into a CoA thioester or a CoA thioester into an ACP thioester,
Eᵥ Wax ester synthase or alcohol O acyltransferase, preferably of EC 2.3.1.75 or EC 2.3.1.84, which catalyses the synthesis of an ester from an acyl-coenzyme A thioester or an ACP thioester and an alcohol,
Eᵥᵢ Acyl-CoA (coenzyme A) synthetase, preferably of EC 6.2.1.3, which catalyses the synthesis of an acyl-coenzyme A thioester, and
Eᵥᵢᵢ Acyl thioesterase, preferably of EC 3.1.2.2, EC 3.1.2.4, EC 3.1.2.18, EC 3.1.2.19, EC 3.1.2.20 or EC 3.1.2.22, which catalyses the conversion of an acyl thioester with an alcohol to a carboxylate ester.

In this connection, it is particularly preferable that the third genetic modification comprises combinations of the increased activities of the enzymes selected from

### Eᵥ, Eᵥᵢᵢ, EᵥEᵥᵢ, EᵥᵢEᵥᵢᵢ and EᵥᵢEᵥᵢᵢE_{iib}.

Preferred enzymes E_{iib} in connection with the third genetic modification correspond to the enzymes E_{iib} listed above as preferable in connection with the first genetic modification.

Specific enzymes Eᵥ

In cells preferred according to the invention, the enzyme Eᵥ is one which comprises sequences selected from:

NP_{—}808414.2, NP_{—}001178653.1, XP_{—}003272721.1, XP_{—}002720111.1, NP_{—}001002254.1, XP_{—}529027.1, XP_{—}002831804.1, BAC28882.1, XP_{—}549056.2, XP_{—}002918053.1, XP_{—}001085075.1, XP_{—}002763005.1, XP_{—}002700092.1, XP_{—}599558.4, EDL95940.1, XP_{—}001496780.1, CAD89267.1, EFB28125.1, YP_{—}004747160.1, YP_{—}004746900.1, YP_{—}004746665.1, YP_{—}004746558.1, YP_{—}004746531.1, YP_{—}004746530.1, YP_{—}004745948.1, YP_{—}004745222.1, YP_{—}004744358.1, YP_{—}004743710.1, YP_{—}002492297.1, AEK40846.1, YP_{—}001847685.1, YP_{—}001712672.1, YP_{—}001706290.1, YP_{—}004724737.1, YP_{—}004723134.1, AEJ51098.1, AEJ48174.1, AEJ47480.1, YP_{—}004392630.1, YP_{—}004099725.1, YP_{—}003912033.1, YP_{—}003652731.1, YP_{—}003301387.1, YP_{—}003298139.1, YP_{—}001509672.1, YP_{—}001505948.1, YP_{—}001432486.1, YP_{—}001432432.1, YP_{—}924893.1, YP_{—}923981.1, YP_{—}922869.1, YP_{—}922597.1, YP_{—}922419.1, ZP_{—}08629145.1, ZP_{—}08628906.1, YP_{—}001380027.1, YP_{—}001280731.1, YP_{—}001280730.1, YP_{—}888966.1, YP_{—}890540.1, YP_{—}888236.1, YP_{—}888223.1, YP_{—}888574.1, YP_{—}884705.1, YP_{—}889488.1, YP_{—}886248.1, YP_{—}882534.1, YP_{—}881069.1, YP_{—}881444.1, YP_{—}883472.1, YP_{—}879642.1, YP_{—}884073.1, YP_{—}880917.1, YP_{—}882201.1, YP_{—}879422.1, YP_{—}707862.1, YP_{—}707847.1, YP_{—}707633.1, YP_{—}707572.1, YP_{—}707571.1, YP_{—}706785.1, YP_{—}706267.1, YP_{—}705586.1, YP_{—}705294.1, YP_{—}702929.1, YP_{—}701572.1, YP_{—}700576.1, YP_{—}700081.1, YP_{—}700033.1, YP_{—}700018.1, YP_{—}700017.1, YP_{—}699999.1, CCB78299.1, CCB78283.1, CCB72233.1, YP_{—}004663601.1, YP_{—}004525283.1, YP_{—}004524901.1, YP_{—}004524237.1, YP_{—}004524223.1, YP_{—}004523752.1, YP_{—}004522677.1, YP_{—}004521797.1, YP_{—}004521441.1, YP_{—}004020500.1, YP_{—}004014348.1, EGO40684.1, EGO38684.1, EGO38655.1, EGO37244.1, EGO36970.1, EGO36701.1, YP_{—}003951335.1, YP_{—}003812176.1, YP_{—}003811992.1, YP_{—}003810691.1, YP_{—}003810418.1, YP_{—}003809501.1, ZP_{—}08574204.1, CCA19760.1, XP_{—}002900672.1, ZP_{—}06414567.1, ZP_{—}06413635.1, ZP_{—}06411773.1, ZP_{—}06411772.1, ZP_{—}06271823.1, ZP_{—}05620754.1, ZP_{—}05360001.1, ZP_{—}04752019.1, ZP_{—}04751943.1, ZP_{—}04750965.1, ZP_{—}04750465.1, ZP_{—}04750453.1, ZP_{—}04750228.1, ZP_{—}04750091.1, ZP_{—}04749363.1, ZP_{—}04749348.1, ZP_{—}04749293.1, ZP_{—}04749287.1, ZP_{—}04749022.1, ZP_{—}04748677.1, ZP_{—}04747379.1, ZP_{—}04747377.1, ZP_{—}04747348.1, ZP_{—}04747282.1, ZP_{—}04747159.1, ZP_{—}04747093.1, ZP_{—}04746958.1, ZP_{—}04717323.1, ZP_{—}04684258.1, ZP_{—}04386203.1, ZP_{—}04385082.1, ZP_{—}04384030.1, ZP_{—}04384029.1, ZP_{—}03534755.1, ZP_{—}01115502.1, ZP_{—}01102322.1, YP_{—}004583872.1, YP_{—}004583323.1, YP_{—}004573656.1, YP_{—}004571392.1, YP_{—}003513699.1, ZP_{—}08553011.1, ZP_{—}08552672.1, YP_{—}003467054.1, YP_{—}003572597.1, YP_{—}579515.1, YP_{—}001136465.1, YP_{—}001136231.1, YP_{—}001135959.1, YP_{—}001135349.1, YP_{—}001133828.1, YP_{—}001133806.1, YP_{—}001133693.1, YP_{—}001133270.1, YP_{—}001132329.1, YP_{—}001131721.1, YP_{—}001131631.1, YP_{—}001073715.1, YP_{—}001073143.1, YP_{—}001072388.1, YP_{—}001072036.1, YP_{—}001071893.1, YP_{—}001071814.1, YP_{—}001071689.1, YP_{—}001070856.1, YP_{—}001069682.1, YP_{—}001069164.1, YP_{—}001068496.1, YP_{—}939377.1, YP_{—}642242.1, YP_{—}641664.1, YP_{—}641419.1, YP_{—}640919.1, YP_{—}640783.1, YP_{—}640704.1, YP_{—}640572.1, YP_{—}640571.1, YP_{—}640494.1, YP_{—}639709.1, YP_{—}639198.1, YP_{—}638523.1, YP_{—}638030.1, YP_{—}637968.1, YP_{—}637380.1, YP_{—}446603.1, NP_{—}001185377.1, NP_{—}200151.2, NP_{—}568547.1, NP_{—}197641.1, NP_{—}200150.1, NP_{—}197139.1, NP_{—}190490.1, NP_{—}190488.1, NP_{—}177356.1, YP_{—}004495408.1, YP_{—}004495023.1, YP_{—}004494197.1, YP_{—}004494168.1, YP_{—}004493973.1, YP_{—}004493936.1, YP_{—}004493628.1, YP_{—}004493589.1, YP_{—}004493509.1, YP_{—}004493477.1, YP_{—}004493462.1, YP_{—}004492352.1, YP_{—}004492155.1, YP_{—}004492039.1, YP_{—}004491716.1, YP_{—}004491715.1, YP_{—}004491501.1, YP_{—}003375642.1, YP_{—}003411203.1, YP_{—}003410436.1, YP_{—}003395271.1, YP_{—}003395089.1, YP_{—}003393635.1, YP_{—}003384208.1, YP_{—}003379551.1, ZP_{—}04388235.1, YP_{—}002134168.1, ZP_{—}01900421.1, ZP_{—}01900085.1, ZP_{—}01899829.1, ZP_{—}01898741.1, BAK05274.1, BAJ93623.1, BAJ97841.1, BAK08349.1, BAJ93204.1, BAJ92722.1, BAK06983.1, BAJ86545.1, BAK02325.1, BAJ85619.1, BAJ84892.1, ZP_{—}05218281.1, ZP_{—}05218149.1, ZP_{—}05217310.1, ZP_{—}05216978.1, ZP_{—}05216447.1, ZP_{—}05216446.1, ZP_{—}05216025.1, ZP_{—}05214687.1, ZP_{—}08476543.1, ZP_{—}04749239.1, YP_{—}823060.1, ADP99639.1, ADP98951.1, ADP98855.1, ADP98710.1, ADP96265.1, ZP_{—}08461736.1, ZP_{—}08461735.1, ZP_{—}07608690.1, YP_{—}045555.1 (encoded by SEQ ID No. 22), YP_{—}872243.1, YP_{—}004009106.1, YP_{—}004008736.1, YP_{—}004008003.1, YP_{—}004007600.1, YP_{—}004006799.1, YP_{—}004006436.1, YP_{—}004006072.1, YP_{—}004005008.1, YP_{—}003486913.1, NP_{—}301898.1, ZP_{—}08434757.1, YP_{—}004079491.1, YP_{—}004078785.1, YP_{—}004077880.1, YP_{—}004076486.1, YP_{—}004076464.1, YP_{—}004076350.1, YP_{—}004075391.1, YP_{—}004074864.1, ZP_{—}01103855.1, YP_{—}465274.1, ZP_{—}08403393.1, ZP_{—}08402717.1, ZP_{—}08402716.1, YP_{—}004427559.1, YP_{—}001277083.1, YP_{—}001276783.1, YP_{—}524767.1, YP_{—}522739.1, YP_{—}521788.1, YP_{—}004335162.1, YP_{—}004333708.1, YP_{—}004332973.1, YP_{—}004332349.1, YP_{—}004157731.1, YP_{—}004224204.1, YP_{—}003275673.1, YP_{—}003275371.1, YP_{—}003274979.1, YP_{—}003274924.1, YP_{—}003274705.1, YP_{—}956544.1, YP_{—}955502.1, YP_{—}955007.1, YP_{—}954887.1, YP_{—}954886.1, YP_{—}954859.1, YP_{—}954399.1, YP_{—}953715.1, YP_{—}953073.1, YP_{—}952592.1, YP_{—}951909.1, YP_{—}951298.1, YP_{—}951083.1, ZP_{—}08287899.1, ZP_{—}08272356.1, ZP_{—}08270967.1, CCA60099.1, CCA56737.1, YP_{—}983728.1, YP_{—}550833.1, YP_{—}549124.1, YP_{—}121795.1, YP_{—}120815.1, YP_{—}118589.1, YP_{—}117783.1, YP_{—}117375.1, YP_{—}003646883.1, YP_{—}003646055.1, YP_{—}003645661.1, EGE49469.1, ZP_{—}08234310.1, CBZ53121.1, YP_{—}004010866.1, EGE24961.1, EGE18726.1, EGE15701.1, EGE12950.1, EGE10026.1, EGB03968.1, ZP_{—}08206563.1, ZP_{—}08205089.1, ZP_{—}08204958.1, ZP_{—}08204416.1, ZP_{—}08203326.1, YP_{—}714381.1, YP_{—}713817.1, YP_{—}694462.1, YP_{—}693524.1, YP_{—}003341775.1, YP_{—}003339587.1, ZP_{—}08197177.1, ADW01905.1, YP_{—}004242683.1, ZP_{—}07484742.2, ZP_{—}07441979.2, ZP_{—}07441978.2, ZP_{—}07437333.2, ZP_{—}06960424.1, ZP_{—}06801236.1, ZP_{—}06799517.1, ZP_{—}05769718.1, ZP_{—}05768326.1, ZP_{—}05767970.1, ZP_{—}05766272.1, ZP_{—}05763839.1, YP_{—}003204265.1, YP_{—}003203570.1, YP_{—}003200768.1, YP_{—}003134884.1, YP_{—}003134608.1, ZP_{—}05140320.1, NP_{—}001140997.1, EEE64643.1, EEE55448.1, EEE32548.1, ZP_{—}03534756.1, ZP_{—}03533653.1, ZP_{—}03531929.1, EEC71274.1, EAY98969.1, EAY75974.1, EAY75973.1, ADZ24988.1, ZP_{—}08157247.1, ZP_{—}08156660.1, ZP_{—}08156249.1, ZP_{—}08153292.1, ZP_{—}08152876.1, ZP_{—}08152662.1, YP_{—}002946672.1, YP_{—}960669.1, YP_{—}960629.1, YP_{—}960328.1, YP_{—}958134.1, YP_{—}957462.1, YP_{—}001022272.1, ZP_{—}08123690.1, ZP_{—}08120547.1, ZP_{—}08119498.1, EGB29195.1, EGB27143.1, YP_{—}003770089.1, YP_{—}003769971.1, YP_{—}003764703.1, YP_{—}003764513.1, YP_{—}003103950.1, YP_{—}003168536.1, YP_{—}003168331.1, YP_{—}003166844.1, CAJ88696.1, NP_{—}769520.1, YP_{—}001141853.1, YP_{—}001108534.1, YP_{—}001106516.1, YP_{—}907824.1, YP_{—}907344.1, YP_{—}906945.1, YP_{—}906856.1, YP_{—}906855.1, YP_{—}906831.1, YP_{—}906494.1, YP_{—}906243.1, YP_{—}905962.1, YP_{—}905765.1, YP_{—}905343.1, YP_{—}905239.1, YP_{—}325796.1, YP_{—}130413.1, NP_{—}625255.1, NP_{—}624462.1, NP_{—}338129.1, NP_{—}338004.1, NP_{—}337859.1, NP_{—}337740.1, NP_{—}337694.1, NP_{—}336266.1, NP_{—}335919.1, NP_{—}335351.1, NP_{—}334638.1, NP_{—}218257.1, NP_{—}218251.1, NP_{—}217997.1, NP_{—}217888.1, NP_{—}217751.1, NP_{—}217750.1, NP_{—}217646.1, NP_{—}217604.1, NP_{—}217603.1, NP_{—}217000.1, NP_{—}216801.1, NP_{—}216276.1, NP_{—}215941.1, NP_{—}215410.1, NP_{—}214735.1, ZP_{—}04661667.1, EFW44815.1, EFW44455.1, ZP_{—}08024634.1, ZP_{—}08024620.1, ZP_{—}08023777.1, ZP_{—}08023597.1, YP_{—}002784032.1, YP_{—}002783585.1, YP_{—}002782904.1, YP_{—}002782647.1, YP_{—}002780099.1, YP_{—}002779887.1, YP_{—}002778497.1, YP_{—}002777657.1, YP_{—}002777402.1, ZP_{—}07966321.1, ZP_{—}07944768.1, CBI21867.3, CBI40547.3, CBI40544.3, CBI40540.3, CBI40536.3, CBI40534.3, CBI40533.3, CBI32385.3, ZP_{—}05765756.1, ZP_{—}05765643.1, ZP_{—}05765597.1, ZP_{—}05765596.1, YP_{—}001705267.1, YP_{—}001704692.1, YP_{—}001704281.1, YP_{—}001702654.1, YP_{—}001701260.1, ZP_{—}05770434.1, ZP_{—}05766274.1, ZP_{—}05762133.1, ZP_{—}05762130.1, ZP_{—}01101223.1, YP_{—}481580.1, YP_{—}979623.1, YP_{—}979196.1, ZP_{—}07414300.2, ZP_{—}03537340.1, ZP_{—}03537339.1, ZP_{—}03536772.1, ZP_{—}03536404.1, ZP_{—}03433478.1, ZP_{—}03430367.1, ZP_{—}03430260.1, ZP_{—}03429345.1, ZP_{—}03428583.1, ZP_{—}03426905.1, ZP_{—}03426458.1, ZP_{—}03426456.1, ZP_{—}03426455.1, ZP_{—}03425014.1, ZP_{—}03424082.1, ZP_{—}03421649.1, ZP_{—}03419291.1, ZP_{—}03418394.1, ZP_{—}03417976.1, ZP_{—}03414875.1, ZP_{—}06952098.1, ZP_{—}05528769.1, ZP_{—}05527907.1, ZP_{—}05227984.1, ZP_{—}05227897.1, ZP_{—}05227653.1, ZP_{—}05227585.1, ZP_{—}05227420.1, ZP_{—}05227202.1, ZP_{—}05226387.1, ZP_{—}05226386.1, ZP_{—}05225355.1, ZP_{—}05225200.1, ZP_{—}05223431.1, ZP_{—}05223402.1, ZP_{—}04697793.1, ZP_{—}02550609.1, ZP_{—}02548969.1, EEE25493.1, ABO13188.2, ZP_{—}07205208.1, YP_{—}589436.1, BAJ33896.1, ZP_{—}07718107.1, ZP_{—}07717513.1, ZP_{—}07717390.1, ZP_{—}07716424.1, ZP_{—}04384387.1, ZP_{—}07376578.1, ZP_{—}06871097.1, ZP_{—}06852444.1, ZP_{—}06852442.1, ZP_{—}06852283.1, ZP_{—}06852150.1, ZP_{—}06852032.1, ZP_{—}06850980.1, ZP_{—}06850766.1, ZP_{—}06850644.1, ZP_{—}06849846.1, ZP_{—}06849446.1, ZP_{—}06849265.1, ZP_{—}06848894.1, ZP_{—}06848550.1, ZP_{—}06847321.1, ZP_{—}06847245.1, ZP_{—}06728640.1, ZP_{—}06155537.1, ZP_{—}03822106.1, ZP_{—}03822105.1, ZP_{—}03264909.1, ZP_{—}01915979.1, ZP_{—}01914209.1, ZP_{—}01909198.1, ZP_{—}01895985.1, ZP_{—}01893763.1, ZP_{—}01893601.1, ZP_{—}01893547.1, ZP_{—}01864269.1, ZP_{—}01736818.1, ZP_{—}01693481.1, ZP_{—}01626518.1, ZP_{—}01616172.1, ZP_{—}01461648.1, ZP_{—}01439861.1, ZP_{—}01311414.1, ZP_{—}01222733.1, ZP_{—}01038993.1, ZP_{—}00997001.1, ZP_{—}06533596.1, ZP_{—}07308012.1, ZP_{—}07282351.1, ZP_{—}07282257.1, ZP_{—}07278697.1, ZP_{—}07277986.1, ZP_{—}07277799.1, ZP_{—}07011797.1, ZP_{—}06913634.1, ZP_{—}06711075.1, ZP_{—}06575037.1, ZP_{—}06523715.1, ZP_{—}06522644.1, ZP_{—}06520408.1, ZP_{—}06518751.1, ZP_{—}06514733.1, ZP_{—}06511304.1, ZP_{—}06510466.1, ZP_{—}06509700.1, ZP_{—}06504004.1, ZP_{—}06452618.1, ZP_{—}06451687.1, ZP_{—}06450049.1, ZP_{—}06444722.1, ZP_{—}06443996.1, ZP_{—}06443677.1, ZP_{—}06438510.1, ZP_{—}06435077.1, ZP_{—}06434554.1, ZP_{—}06432969.1, ZP_{—}06431341.1, ZP_{—}06430915.1, ZP_{—}05129423.1, ZP_{—}05127637.1, ZP_{—}05126217.1, ZP_{—}05096686.1, ZP_{—}05095013.1, ZP_{—}05094400.1, ZP_{—}05093434.1, ZP_{—}05043539.1, ZP_{—}05041631.1, ZP_{—}04959394.1, ZP_{—}04956551.1, ZP_{—}01052702.1, YP_{—}437020.1, YP_{—}436128.1, YP_{—}432512.1, YP_{—}432391.1, ZP_{—}06072118.1, ZP_{—}06069021.1, ZP_{—}06065092.1, ZP_{—}06062254.1, YP_{—}003032200.1, YP_{—}003030813.1, YP_{—}002766854.1, YP_{—}002766842.1, YP_{—}002766292.1, YP_{—}002765623.1, YP_{—}002765076.1, YP_{—}002764977.1, YP_{—}002764976.1, YP_{—}002764693.1, YP_{—}002764633.1, YP_{—}002646305.1, YP_{—}002646304.1, YP_{—}001853537.1, YP_{—}001853530.1, YP_{—}001853214.1, YP_{—}001852100.1, YP_{—}001851711.1, YP_{—}001851686.1, YP_{—}001851684.1, YP_{—}001851611.1, YP_{—}001851610.1, YP_{—}001851579.1, YP_{—}001850950.1, YP_{—}001850935.1, YP_{—}001850900.1, YP_{—}001850899.1, YP_{—}001850378.1, YP_{—}001849911.1, YP_{—}001849825.1, YP_{—}001849624.1, YP_{—}001849470.1, YP_{—}001848848.1, YP_{—}001848784.1, YP_{—}001822237.1, YP_{—}001289190.1, YP_{—}001289078.1, YP_{—}001288434.1, YP_{—}001287727.1, YP_{—}001286168.1, YP_{—}001085790.1, YP_{—}856793.1, YP_{—}629387.1, YP_{—}615587.1, YP_{—}615252.1, YP_{—}457389.1, YP_{—}263530.1, NP_{—}962591.1, NP_{—}962411.1, NP_{—}962281.1, NP_{—}961234.1, NP_{—}960903.1, NP_{—}960387.1, NP_{—}960090.1, NP_{—}959281.1, NP_{—}959065.1, NP_{—}857403.1, NP_{—}857149.1, NP_{—}857148.1, NP_{—}857047.1, NP_{—}856907.1, NP_{—}856759.1, NP_{—}856156.1, NP_{—}855443.1, NP_{—}855112.1, NP_{—}853892.1, NP_{—}828432.1, NP_{—}603766.1, XP_{—}003081224.1, YP_{—}003778608.1, YP_{—}003730939.1, XP_{—}003059244.1, ADI13131.1, XP_{—}002992800.1, XP_{—}002963877.1, XP_{—}001419779.1, XP_{—}002988280.1, XP_{—}002987493.1, CBH32551.1, CBH32550.1, CBH19575.1, CBH19574.1, YP_{—}003627553.1, XP_{—}002879777.1, XP_{—}002877657.1, XP_{—}002877655.1, XP_{—}002873570.1, XP_{—}002871716.1, XP_{—}002870738.1, XP_{—}002868506.1, XP_{—}002865972.1, XP_{—}002864239.1, XP_{—}002862308.1, ZP_{—}05823139.1, NP_{—}001043877.1, ZP_{—}06693274.1, ZP_{—}06058985.1, NP_{—}001044374.1, XP_{—}002835451.1, XP_{—}002787542.1, XP_{—}002785958.1, XP_{—}002785645.1, XP_{—}002783220.1, XP_{—}002774061.1, XP_{—}002767852.1, XP_{—}002766051.1, XP_{—}002765456.1, XP_{—}002765455.1, XP_{—}002677788.1, XP_{—}002671612.1, XP_{—}002736281.1, CBA31373.1, XP_{—}002184474.1, XP_{—}002325936.1, XP_{—}002323705.1, XP_{—}002325937.1, XP_{—}002323911.1, XP_{—}002323706.1, XP_{—}002328965.1, XP_{—}002318416.1, XP_{—}002310400.1, ACY38597.1, ACY38596.1, ACY38595.1, ACY38594.1, ACY38593.1, ACY38592.1, ACY38591.1, ACY38590.1, ACX81315.1, ACX81314.1, XP_{—}001868729.1, XP_{—}001847517.1, XP_{—}001847515.1, XP_{—}002502575.1, ACU20370.1, ACU18073.1, XP_{—}002523348.1, XP_{—}002516707.1, XP_{—}002429016.1, BAH89673.1, XP_{—}002440221.1, XP_{—}002459294.1, XP_{—}002458560.1, XP_{—}320167.4, XP_{—}001780431.1, XP_{—}002364905.1, XP_{—}002263196.1, XP_{—}002263137.1, XP_{—}002263409.1, XP_{—}002263252.1, XP_{—}002268615.1, XP_{—}002278404.1, XP_{—}002274522.1, XP_{—}002282418.1, XP_{—}001633379.1, XP_{—}001632267.1, XP_{—}001632004.1, XP_{—}001622638.1, XP_{—}002155609.1, XP_{—}759225.1, XP_{—}002152406.1, XP_{—}001914129.1, XP_{—}001738032.1, XP_{—}001731626.1, XP_{—}001209859.1, CAN79451.1, CAN78449.1, CAN72806.1, CAN71951.1, CAN71950.1, CAN76656.1, CAN62907.1, AAZ08051.1, ABO21022.1, ABO21021.1, ABO21020.1, ABJ96321.1, BAF01088.1, XP_{—}758106.1, BAC42871.1, BAB09801.1, BAB09102.1, in particular YP_{—}045555.1 (encoded by SEQ ID No. 22) and NP_{—}808414.2
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme Eᵥ in general is understood to mean in particular the conversion of dodecanoyl-CoA thioester and/or dodecanoyl-ACP thioester with methanol to dodecanoyl methyl esters.

If the enzyme Eᵥ is an alcohol O-acyltransferase of EC 2.3.1.84, then it is preferable that this is selected from:

EGA72844.1, NP_{—}015022.1, S69991, AAP72991.1, EDN63695.1, BAA05552.1, AAP72992.1, S69992, AAP72995.1, XP_{—}002552712.1, XP_{—}001646876.1, XP_{—}002551954.1, EGA82692.1, EDN61766.1, EGA86689.1, EGA74966.1, AAU09735.1, NP_{—}011693.1, XP_{—}445666.1, BAA13067.1, AAP72993.1, EGA62172.1, XP_{—}455762.1, EGA58658.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme Eᵥ in general is understood to mean in particular the conversion of dodecanoyl-CoA thioester with methanol to dodecanoyl methyl ester.

Microorganisms preferred according to the invention are those which are obtained when the microorganisms listed below having a third genetic modification in the sense of the invention are used as the starting point, in that they are provided with a first and second genetic modification and optionally at least one further genetic modification in the sense of the invention.

WO2007136762 A2 describes, particularly on pages 2 to 4 and 21 to 24, FIGS. 2 to 4, practical examples 1, 2 and 5 to 7 and claims 1, 2, 5, 6, 9 to 27 and 33, microorganisms preferably used according to the invention which have a third genetic modification, so that compared to their wild type they are capable of forming more fatty acids and fatty acid derivatives, in particular fatty acid esters, wax esters, hydrocarbons and fatty alcohols, from at least one simple carbon source. The document also describes enzymes Eᵢ preferred according to the invention and the sequences thereof, particularly on pages 21 to 24, in table 10 and FIG. 10.

Specific enzymes Eᵥᵢ

In cells preferred according to the invention, the enzyme Eᵥᵢ is one which comprises sequences selected from YP_{—}001724804.1 (encoded by SEQ ID No. 21)

and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequence are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme Eᵥᵢ in general is understood to mean in particular the synthesis of dodecanoyl-CoA thioester.

### Specific Enzymes Eᵥᵢᵢ

Microorganisms preferred according to the invention are those which are obtained when the microorganisms listed below having a third genetic modification in the sense of the invention are used as the starting point, in that they are provided with a first and second genetic modification and optionally at least one further genetic modification in the sense of the invention.

WO2010075483 A2 describes, particularly in paragraphs [0061] to [0090] and [0287] to [0367], FIGS. 1, 4 and 5, practical examples 1 to 38 and claims 18 to 26, microorganisms preferably used according to the invention which have a third genetic modification, so that compared to their wild type they are capable of forming more fatty acids and fatty acid derivatives, in particular fatty acids, fatty acid methyl esters, fatty acid ethyl esters, fatty alcohols, fatty alkyl acetates, fatty aldehydes, fatty amines, fatty amides, fatty sulphates, fatty ethers, ketones, alkanes, internal and terminal olefins, dicarboxylic acids, α,ω-dicarboxylic acids and α,ω-diols, from at least one simple carbon source. The document also describes enzymes Eᵢ preferred according to the invention and the sequences thereof, particularly in paragraphs [0012] to [0060], tables 7, 17, 26 and 27, FIGS. 1, 44 to 47 and 55 to 59, practical examples 1 to 38 and claims 1 to 17.

### Fourth Genetic Modification for the Production of ω-Hydroxy- or ω-Oxo-Functionalized Carboxylic Acids and Esters

For the case where the production of ω-hydroxy- or ω-oxo-functionalized carboxylic acids and esters—also as precursor stages for further ω-functionalizations such as for example ω-amination—is desired, it can be advantageous suitably to reduce the corresponding carboxylic acids or esters which have been oxidized in the ω position up to the carboxy function in the microorganism.

For this, microorganisms preferred according to the invention have a fourth genetic modification which, compared with the enzymatic activity of the wild type of the microorganism, comprises increased activity of at least one of the enzymes selected from the group

E_{iib} Acyl-CoA (coenzyme A):ACP (acyl carrier protein) transacylase, which converts an ACP thioester into a CoA thioester or a CoA thioester into an ACP thioester,
Eᵥᵢ Acyl-CoA (coenzyme A) synthetase, preferably of EC 6.2.1.3, which preferentially catalyses the synthesis of an acyl-coenzyme A thioester,
Eᵥᵢᵢ Acyl-CoA (coenzyme A) reductase, preferably of EC 1.2.1.42 or EC 1.2.1.50, which preferentially catalyses the reduction of an acyl-coenzyme A thioester to the corresponding alkan-1-al or alkan-1-ol
Eᵢₓ Fatty acid reductase (also fatty aldehyde dehydrogenase or arylaldehyde oxidoreductase), preferably of EC 1.2.1.3, EC 1.2.1.20 or EC 1.2.1.48, which preferentially catalyses the reduction of an alkanoic acid to the corresponding alkan-1-al, and
Eₓ Acyl-ACP (acyl carrier protein) reductase, preferably of EC 1.2.1.80, which preferentially catalyses the reduction of an acyl-ACP thioester to the corresponding alkan-1-al or alkan-1-ol.

In this connection, it is particularly preferable that the fourth genetic modification comprises combinations of increased activities of the enzymes selected from Eᵥᵢᵢᵢ, Eᵢₓ, Eₓ, EᵥᵢEᵥᵢᵢᵢ and EᵥᵢEₓE_{iib}.

Preferred enzymes E_{iib} in connection with the fourth genetic modification correspond to the enzymes E_{iib} listed above as preferable in connection with the first and third genetic modification.

Thus configured organisms preferred according to the invention are also outstandingly suitable for the production of compounds which are selected from α,ω-alkanediols, α,ω-alkanedialdehydes, α-oxo-ω-hydroxyalkanes and α,ω-alkanediamines, α,ω-alkenediols, α,ω-alkenedialdehydes, α-oxo-ω-hydroxyalkenes and α,ω-alkenediamines, since compounds of these classes are produced in significant quantities as well as the ω-functionalized carboxylic acids and ω-functionalized carboxylate esters.

In this connection, it may be mentioned that the alkene derivatives in particular arise through the conversion of unsaturated fatty acids formed by the microorganism, such as for example palmitoleic acid, oleic acid, linolic acid, adinolenic acid and γ-linolenic acid.

### Specific Enzymes Eᵥᵢᵢᵢ

Microorganisms preferred according to the invention are those which are obtained when the microorganisms listed below having a fourth genetic modification in the sense of the invention are used as the starting point, in that they are provided with a first and second genetic modification and optionally at least one further genetic modification in the sense of the invention.

WO2011008565 A1 describes, particularly in paragraphs [0021], [0103] to [0106], [0108] and [0129], microorganisms preferably used according to the invention which have a fourth genetic modification, so that compared to their wild type they are capable of forming more fatty acids and fatty acid derivatives, in particular fatty acids, fatty aldehydes, fatty alcohols, alkanes and fatty acid esters from at least one simple carbon source. The document also describes enzymes Eᵥᵢᵢᵢ preferred according to the invention and the sequences thereof, particularly in paragraphs [0104] to [0106] and [0108] and [0129] and practical example 11.

WO2008151149 A2 describes, particularly in paragraphs [0009], [0015] to [0037], [0053], [0071], [0171], [0174] to [0191], [0274] and [0396], claims 53 to 114, 188 to 206 and 344 to 355 and tables 1 to 3, microorganisms preferably used according to the invention which have a fourth genetic modification, so that compared to their wild type they are capable of forming more microbial oil from at least one simple carbon source. The document also describes enzymes Eᵥᵢᵢᵢ preferred according to the invention and the sequences thereof, particularly in paragraphs [0255] to [0261] and [0269] and tables 6 and 7.

WO2007136762 A2 describes, particularly on pages 2 to 4 and 19 to 20, FIGS. 2 to 4, practical examples 2 to 7 and claims 4, 8 to 27 and 33, microorganisms preferably used according to the invention which have a fourth genetic modification, so that compared to their wild type they are capable of forming more fatty acids and fatty acid derivatives, in particular fatty acid esters, wax esters, hydrocarbons and fatty alcohols, from at least one simple carbon source. The document also describes enzymes Eᵥᵢᵢᵢ preferred according to the invention and the sequences thereof, particularly on pages 19 to 20, in table 10 and FIG. 10.

WO2011019858 A1 describes, particularly in paragraphs [0015] to [0020], [0064] to [0074], [0085] to [0086] and [0092] to [0099], practical examples 1 to 13, FIG. 1 and claims 1 to 14, microorganisms preferably used according to the invention which have a fourth genetic modification, so that compared to their wild type they are capable of forming more fatty acids and fatty acid derivatives, in particular fatty alcohols, from at least one simple carbon source. The document also describes enzymes Eᵥᵢᵢᵢ preferred according to the invention and the sequences thereof, particularly in paragraphs [0004] to [0007] and [0075] to [0080] and practical examples 1 to 13.

WO2009140695 A1 describes, particularly in paragraphs [0031] to [0040], [0051] and [0214] to [0233], practical examples 22 to 24, table 1, FIG. 40, practical examples 5 to 24 and 28 to 30 and claims 29 to 30, microorganisms preferably used according to the invention which have a fourth genetic modification, so that compared to their wild type they are capable of forming more fatty acids and fatty acid derivatives, in particular hydrocarbons, from at least one simple carbon source. The document also describes enzymes Eᵥᵢᵢᵢ preferred according to the invention and the sequences thereof, particularly in paragraphs [0023] to [0030], [0056], [0066] to [0069] and [0193] to [0208], table 1, FIG. 39, practical examples 5 to 24 and 28 to 30 and claims 69 to 74.

WO2011008535 A1 describes, particularly in paragraphs [0023] to [0024], and [0133] to [0158], FIG. 13, claims 39 and 45 to 47 and practical examples 1 to 5, microorganisms preferably used according to the invention which have a fourth genetic modification, so that compared to their wild type they are capable of forming more fatty acids and fatty acid derivatives, in particular carboxylic acids, hydroxycarboxylic acids and lactones thereof from at least one simple carbon source. The document also describes enzymes Eᵥᵢᵢᵢ preferred according to the invention and the sequences thereof, particularly in paragraphs [0017] to [0022], [0084] to [0132], FIGS. 2 to 12, claims 31 to 37 and 40 to 44 and practical examples 1 to 5.

WO2010063031 A2 describes, particularly in paragraphs [0007], [0092] to [0100], [0181] to [0183] and [0199] to [0213], microorganisms preferably used according to the invention which have a fourth genetic modification, so that compared to their wild type they are capable of forming more microbial oil from at least one simple carbon source. The document also describes enzymes Eᵥᵢᵢᵢ preferred according to the invention and the sequences thereof, particularly in paragraphs [0191] to [0194] and tables 4 and 5.

WO2010063032 A2 describes, particularly in paragraphs [0007], [0092] to [0100], [0181] to [0183] and [0199] to [0213], microorganisms preferably used according to the invention which have a fourth genetic modification, so that compared to their wild type they are capable of forming more microbial oil from at least one simple carbon source. The document also describes enzymes Eᵥᵢᵢᵢ preferred according to the invention and the sequences thereof, particularly in paragraphs [0191] to [0194] and tables 4 and 5.

### Specific Enzymes Eᵢₓ

Microorganisms preferred according to the invention are those which are obtained when the microorganisms listed below having a fourth genetic modification in the sense of the invention are used as the starting point, in that they are provided with a first and second genetic modification and optionally at least one further genetic modification in the sense of the invention.

WO2011019858 A1 describes, particularly in paragraphs [0004] to [0008], [0064] to [0074], [0085] to [0086], [0095] to [0099], practical examples 1 to 13, FIG. 1 and claim 7, microorganisms preferably used according to the invention which have a fourth genetic modification, so that compared to their wild type they are capable of forming more fatty acids and fatty acid derivatives, in particular fatty alcohols, from at least one simple carbon source. The document also describes enzymes Eᵢₓ preferred according to the invention and the sequences thereof, particularly in paragraphs [0008] to [0009], [0074] and [0081] to [0082] and practical examples 1 to 13.

WO2010135624 A2 describes, particularly in paragraphs [0005], [0067] to [0085] and [0092] to [0102], claims 13 to 17 and practical examples 1 to 4, microorganisms preferably used according to the invention which have a fourth genetic modification, so that compared to their wild type they are capable of forming more fatty acids and fatty acid derivatives, in particular carboxylic acids, hydroxycarboxylic acids and lactones thereof, from at least one simple carbon source. The document also describes enzymes E₁ preferred according to the invention and the sequences thereof, particularly in paragraphs [0005] to [0006] and [0086] to [0090], FIGS. 3 to 7, claim 28 and practical examples 1 to 4.

WO2010062480 A2 describes, particularly in paragraphs [0022] to [0174] and [0292] to [0316], practical examples 1 and 3 to 8, FIG. 9 and claims 17 and 24, microorganisms preferably used according to the invention which have a fourth genetic modification, so that compared to their wild type they are capable of forming more fatty acids and fatty acid derivatives, in particular fatty alcohols, from at least one simple carbon source. The document also describes enzymes Eᵢₓ preferred according to the invention and the sequences thereof, particularly in paragraphs [0019] to [0032] and [0263] to [0286], table 1, FIGS. 6 to 8 and practical examples 1 and 3 to 8.

WO201042664 A2 describes, particularly in paragraphs [0236] to [0261], practical example 2, FIGS. 1 and 5 and claim 25, microorganisms preferably used according to the invention which have a fourth genetic modification, so that compared to their wild type they are capable of forming more fatty acids and fatty acid derivatives, in particular fatty alcohols, from at least one simple carbon source. The document also describes enzymes Eᵢₓ preferred according to the invention and the sequences thereof, particularly in paragraphs [0211] to [0233], FIGS. 2 to 4 and practical examples 1 to 2.

### Specific Enzymes Eₓ

Microorganisms preferred according to the invention are those which are obtained when the microorganisms listed below having a fourth genetic modification in the sense of the invention are used as the starting point, in that they are provided with a first and second genetic modification and optionally at least one further genetic modification in the sense of the invention.

WO2007136762 A2 describes, particularly on pages 2 to 4 and 19 to 20, FIGS. 2 to 4, practical examples 2 to 7 and claims 4, 8 to 27 and 33, microorganisms preferably used according to the invention which have a fourth genetic modification, so that compared to their wild type they are capable of forming more fatty acids and fatty acid derivatives, in particular fatty acid esters, wax esters, hydrocarbons and fatty alcohols, from at least one simple carbon source. The document also describes enzymes Eₓ preferred according to the invention and the sequences thereof, particularly on pages 19 to 20, in table 10 and FIG. 10.

WO2011019858 A1 describes, particularly in paragraphs [0015] to [0020], [0064] to [0074], [0085] to [0086] and [0092] to [0099], practical examples 1 to 13, FIG. 1 and claims 1 to 14, microorganisms preferably used according to the invention which have a fourth genetic modification, so that compared to their wild type they are capable of forming more fatty acids and fatty acid derivatives, in particular fatty alcohols, from at least one simple carbon source. The document also describes enzymes Eₓ preferred according to the invention and the sequences thereof, particularly in paragraphs [0004] to [0007] and [0075] to [0080] and practical examples 1 to 13.

WO2009140695 A1 describes, particularly in paragraphs [0031] to [0040], [0051] and [0214] to [0233], practical examples 22 to 24, table 1, FIG. 40, practical examples 5 to 24 and 28 to 30 and claim 29, microorganisms preferably used according to the invention which have a fourth genetic modification, so that compared to their wild type they are capable of forming more fatty acids and fatty acid derivatives, in particular hydrocarbons, from at least one simple carbon source. The document also describes enzymes Eₓ preferred according to the invention and the sequences thereof, particularly in paragraphs [0023] to [0030], [0056], [0066] to [0069] and [0193] to [0208], table 1, FIG. 39, practical examples 5 to 24 and 28 to 30 and claims 69 to 74.

WO2011008535 A1 describes, particularly in paragraphs [0023] to [0024], and [0133] to [0158], FIG. 13, claims 39 and 45 to 47 and practical examples 1 to 5, microorganisms preferably used according to the invention which have a fourth genetic modification, so that compared to their wild type they are capable of forming more fatty acids and fatty acid derivatives, in particular carboxylic acids, hydroxycarboxylic acids and lactones thereof, from at least one simple carbon source. The document also describes enzymes Eₓ preferred according to the invention and the sequences thereof, particularly in paragraphs [0017] to [0022], [0084] to [0132], FIGS. 2 to 12, claims 31 to 37 and 40 to 44 and practical examples 1 to 5.

### Fifth Genetic Modification for Suppression of the Degradation of Carboxylic Acids- and Carboxylic Acid Derivatives

Also preferable according to the invention are microorganisms which have a fifth genetic modification which comprises, compared with the enzymatic activity of the wild type of the microorganism, decreased activity of at least one of the enzymes selected from the group

Eₐ Acyl-CoA synthetase, preferably of EC 6.2.1.3, which catalyses the synthesis of an acyl-coenzyme A thioester,
E_{b} Acyl-CoA dehydrogenase, preferably of EC 1.3.99.-, EC 1.3.99.3, or EC 1.3.99.13, which catalyses the oxidation of an acyl-coenzyme A thioester to the corresponding enoyl-coenzyme A thioester,
E_{c} Acyl-CoA-oxidase, preferably of EC 1.3.3.6, which catalyses the oxidation of an acyl-coenzyme A thioester to the corresponding enoyl-coenzyme A thioester,
E_{d} Enoyl-CoA hydratase, preferably of EC 4.2.1.17 or EC 4.2.1.74, which catalyses the hydration of an enoyl-coenzyme A thioester to the corresponding 3-hydroxyacyl-coenzyme A thioester,
Eₑ 3-hydroxyacyl-CoA dehydrogenase, preferably of EC 1.1.1.35 or EC 1.1.1.211, which catalyses the oxidation of a 3-hydroxyacyl-coenzyme A thioester to the corresponding 3-oxoacyl-coenzyme A thioester and
E_{f} Acetyl-CoA acyltransferase, preferably of EC 2.3.1.16, which catalyses the transfer of an acyl residue from a 3-oxoacyl-coenzyme A thioester to coenzyme A and thus creates an acyl-coenzyme A thioester shortened by two carbon atoms.

This has the technical effect that depletion of the carboxylic acids and carboxylate esters formed in increased quantity through the first genetic modification, but also of the ω-functionalized carboxylic acids and carboxylate esters formed in increased quantity through the second, third and fourth genetic modification, is prevented.

### Specific Enzymes Eₐ

It is also preferable according to the invention that the enzyme Eₐ in the cells according to the invention is one which comprises the sequence NP 416319.1.

and
proteins with a polypeptide sequence wherein up to 60%, preferably up to 25%, particularly preferably up to 15%, in particular up to 10, 9, 8, 7, 6, 5, 4, 3, 2 or 1% of the amino acid residues are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme Eₐ in general is understood to mean in particular the synthesis of dodecanoyl-CoA thioester.

### Specific Enzymes E_{b}

It is also preferable according to the invention that in the cells according to the invention the enzyme E_{b} is one which comprises sequences selected from:

AP_{—}000876.1, ZP_{—}08341828.1, YP_{—}002291517.1, ZP_{—}08393771.1, EFW53921.1, YP_{—}003227327.1, YP_{—}001461409.1, AEG35025.1, YP_{—}002385739.1, EGJ00024.1, ZP_{—}08352177.1, ZP_{—}03070250.1, ZP_{—}08367389.1, EGM63466.1, CBI99746.1, ZP_{—}06660773.1, ZP_{—}08372569.1, YP_{—}309282.2, YP_{—}001879017.1, YP_{—}003497883.1, ACI71032.1, YP_{—}002406464.1, EGB79412.1, EFZ76765.1, ZP_{—}07145000.1, ZP_{—}07151031.1, AAZ87047.1, EFZ56676.1, ZP_{—}06656148.1, EGB35012.1, EGB71054.1, EFW49392.1, ZP_{—}07183316.1, YP_{—}002396328.1, YP_{—}002327805.1, ZP_{—}03027602.1, AAG54546.1, YP_{—}001742341.1, ZP_{—}04538240.1, EFX12717.1, ACI71029.1, NP_{—}285938.2, ZP_{—}03064986.1, ZP_{—}07120505.1, YP_{—}539295.2, ZP_{—}03049609.1, ZP_{—}06652178.1, AAP15817.1, NP_{—}706224.3, ABI99723.1, EGB60663.1, EFW71911.1, EGH38574.1, YP_{—}668186.1, EGK29152.1, EGC05203.1, ZP_{—}02801146.2, YP_{—}687886.1, ZP_{—}08346491.1, EGJ94671.1, EGC94003.1, ZP_{—}08362542.1, YP_{—}002381459.1, AAN78852.1, NP_{—}752308.2, ZP_{—}07173894.1, ZP_{—}08357265.1, ZP_{—}08382276.1, ZP_{—}02902298.1, ZP_{—}04560694.1, ZP_{—}06354226.1, CBY94356.1, NP_{—}459307.1, EGE28353.1, ZP_{—}04657138.1, YP_{—}002225434.1, YP_{—}002635948.1, YP_{—}151638.1, ZP_{—}02663643.1, NP_{—}454921.1, YP_{—}004729161.1, YP_{—}215297.1, YP_{—}001454506.1, YP_{—}001571699.1, YP_{—}003363866.1, EGK30199.1, EGJ91900.1, EGK28208.1, ZP_{—}08497358.1, CBK85993.1, YP_{—}003611563.1, YP_{—}004592567.1, YP_{—}003441061.1, YP_{—}002240296.1, YP_{—}002917946.1, ZP_{—}06549304.1, ZP_{—}06017126.1, YP_{—}001333918.1, AAM28523.1, ZP_{—}08305363.1, YP_{—}001439185.1, EGL74026.1, YP_{—}001175495.1, ZP_{—}05968792.1, YP_{—}003209204.1, YP_{—}003943022.1, YP_{—}004499335.1, ZP_{—}06191708.1, YP_{—}001477183.1, ZP_{—}07951567.1, YP_{—}003740265.1, NP_{—}668276.1, ZP_{—}04637564.1, ZP_{—}04631714.1, CBY26031.1, YP_{—}004297237.1, YP_{—}001007400.1, ZP_{—}04625511.1, YP_{—}069424.1, ZP_{—}04616432.1, ZP_{—}04639135.1, YP_{—}001871363.1, ZP_{—}04620883.1, ZP_{—}06636999.1, ZP_{—}07377275.1, YP_{—}003929932.1, YP_{—}001722031.1, ZP_{—}04614013.1, ZP_{—}04628476.1, YP_{—}003713213.1, YP_{—}003530236.1, CBX79727.1, YP_{—}004114694.1, YP_{—}001908526.1, ADP11689.1, YP_{—}002649711.1, YP_{—}003469212.1, YP_{—}003519171.1, YP_{—}051564.1, ZP_{—}03833764.1, ZP_{—}03827249.1, NP_{—}928504.1, YP_{—}004211704.1, ZP_{—}07681706.1, YP_{—}003018849.1, YP_{—}003260788.1, YP_{—}003042091.1, ZP_{—}05973896.1, ZP_{—}03317495.1, ZP_{—}02958330.2, EFW60358.1, EGI98786.1, ZP_{—}06127315.2, YP_{—}002150121.1, ZP_{—}03842196.1, YP_{—}003884303.1, YP_{—}003003248.1, YP_{—}003334792.1, ZP_{—}03379559.1, CBA73629.1, YP_{—}002986552.1, ZP_{—}06538530.1, ZP_{—}01258771.1, ZP_{—}04921840.1, ZP_{—}06180371.1, ZP_{—}08308836.1, ZP_{—}06174994.1, YP_{—}001446380.1, ZP_{—}01237449.1, ZP_{—}01161468.1, ZP_{—}01222040.1, ZP_{—}06038476.1, ZP_{—}05925639.1, ZP_{—}06154677.1, ZP_{—}02195704.1, ZP_{—}01989646.1, ZP_{—}01868523.1, YP_{—}131060.1, ZP_{—}05722161.1, ZP_{—}05716057.1, NP_{—}798668.1, EGF45205.1, ZP_{—}05120764.1, EGR07881.1, ZP_{—}08100412.1, ZP_{—}04919383.1, ZP_{—}06054287.1, YP_{—}002156761.1, YP_{—}205315.2, ZP_{—}04961417.1, ZP_{—}06050299.1, ZP_{—}08103013.1, ZP_{—}01949008.1, NP_{—}231862.1, AEA79156.1, ZP_{—}06081122.1, ZP_{—}04418155.1, YP_{—}001217747.1, ZP_{—}04413631.1, NP_{—}935312.1, ZP_{—}01977990.1, NP_{—}760770.1, YP_{—}004188005.1, YP_{—}002810906.1, ZP_{—}05884155.1, ZP_{—}05946273.1, ZP_{—}01065180.1, ZP_{—}01815735.1, YP_{—}002417909.1, YP_{—}002263750.1, YP_{—}856109.1, ZP_{—}07744057.1, ZP_{—}08520214.1, ZP_{—}06034047.1, YP_{—}004565576.1, ZP_{—}05881167.1, ZP_{—}00991316.1, YP_{—}734276.1, ADT86286.1, YP_{—}001142550.1, YP_{—}869958.1, ZP_{—}08566610.1, ZP_{—}05876732.1, YP_{—}001366225.1, YP_{—}001094233.1, ADV54653.1, YP_{—}963612.1, YP_{—}738268.1, YP_{—}001502248.1, YP_{—}004391846.1, YP_{—}002311644.1, YP_{—}002358241.1, YP_{—}001050670.1, ZP_{—}07390237.1, YP_{—}001674114.1, YP_{—}001554497.1, NP_{—}718122.1, YP_{—}001760976.1, YP_{—}927745.1, YP_{—}562771.1, YP_{—}003557130.1, ZP_{—}02159449.1, YP_{—}003913548.1, YP_{—}001473736.1, YP_{—}750554.1, ZP_{—}01897495.1, YP_{—}268985.1, ZP_{—}01042474.1, ZP_{—}08570996.1, YP_{—}004427315.1, ZP_{—}07010199.1, YP_{—}156047.1, ZP_{—}07097521.1, YP_{—}004467113.1, ZP_{—}01614110.1, YP_{—}340459.1, YP_{—}004434754.1, YP_{—}662062.1, YP_{—}004068195.1, ZP_{—}08409704.1, ZP_{—}08622396.1, ZP_{—}01135962.1, ZP_{—}03560927.1, ZP_{—}04716612.1, EGB41427.1, EGP48304.1, EFV84045.1, ZP_{—}08505249.1, ZP_{—}06688896.1, YP_{—}003980530.1, YP_{—}003168652.1, YP_{—}003146346.1, YP_{—}001250478.1, YP_{—}095752.1, YP_{—}124009.1, CBW99992.1, YP_{—}284763.1, YP_{—}127029.1, YP_{—}746940.1, ZP_{—}07663653.1, ZP_{—}03349444.1, YP_{—}002354470.1, YP_{—}004145615.1, YP_{—}003524477.1, ZP_{—}03698069.1, YP_{—}003376672.1, ZP_{—}06188282.1, EFW81359.1, EGH83675.1, EGH67821.1, EFW83732.1, YP_{—}273865.1, NP_{—}902393.1, ZP_{—}06457469.1, EGH99235.1, ZP_{—}03397893.1, ZP_{—}07004262.1, ZP_{—}06732661.1, ZP_{—}07263971.1, EGH75297.1, NP_{—}888341.1, EGH31566.1, EGH45251.1, NP_{—}643363.1, EGH24154.1, EGH92666.1, EGH73945.1, EGH12424.1, NP_{—}793629.1, ZP_{—}06705890.1, YP_{—}234714.1, EGH62932.1, EGH52925.1, ZP_{—}01126966.1, NP_{—}841588.1, ZP_{—}05109483.1, YP_{—}003847638.1, YP_{—}004294524.1, ZP_{—}02244088.1, NP_{—}884586.1, ZP_{—}08176463.1, ZP_{—}04588788.1, YP_{—}450732.1, ZP_{—}08185386.1, YP_{—}001914265.1, YP_{—}003527565.1, YP_{—}004696148.1, NP_{—}638218.1, ZP_{—}05046817.1, YP_{—}343737.1, ZP_{—}07652844.1, YP_{—}004227922.1, YP_{—}364921.1, YP_{—}001632020.1, NP_{—}744048.1, YP_{—}001898007.1, YP_{—}003145987.1, YP_{—}558241.1, YP_{—}410795.1, YP_{—}001895310.1, YP_{—}002980410.1, ZP_{—}06841648.1, YP_{—}258889.1, YP_{—}931967.1, YP_{—}003760619.1, YP_{—}002029446.1, YP_{—}004474743.1, YP_{—}158312.1, YP_{—}004380764.1, YP_{—}001973352.1, CBJ39115.1, YP_{—}349912.1, YP_{—}003753442.1, ZP_{—}05135288.1, YP_{—}004700980.1, YP_{—}927690.1, YP_{—}001269130.1, YP_{—}742956.1, ADR61321.1, YP_{—}001347709.1, YP_{—}004355482.1, YP_{—}003907207.1, NP_{—}251505.1, ZP_{—}04929120.1, NP_{—}518658.1, YP_{—}002871500.1, ZP_{—}01451059.1, EGM21899.1, YP_{—}001187411.1, ZP_{—}08570514.1, ZP_{—}07794119.1, YP_{—}004391835.1, YP_{—}002256385.1, ZP_{—}07774414.1, YP_{—}855885.1, YP_{—}563120.1, YP_{—}001172167.1, YP_{—}004713921.1, ZP_{—}08138366.1, AEA83572.1, YP_{—}003746704.1, ZP_{—}08521441.1, ZP_{—}05061205.1, YP_{—}001667709.1, YP_{—}750573.1, YP_{—}607261.1, ZP_{—}05118288.1, YP_{—}002311716.1, NP_{—}718079.1, YP_{—}003777020.1, ZP_{—}06052248.1, ZP_{—}00943163.1, ZP_{—}08309312.1, AEG70141.1, YP_{—}001748377.1, YP_{—}001857928.1, YP_{—}001094176.1, YP_{—}003604813.1, ZP_{—}01947893.1,
in particular
EFW81359.1, EGH83675.1, EGH67821.1, EFW83732.1, YP_{—}273865.1, ZP_{—}06457469.1, EGH99235.1, ZP_{—}03397893.1, ZP_{—}07004262.1, ZP_{—}07263971.1, EGH75297.1, EGH31566.1, EGH45251.1, EGH24154.1, EGH92666.1, EGH73945.1, EGH12424.1, NP_{—}793629.1, YP_{—}234714.1, EGH62932.1, EGH52925.1, ZP_{—}04588788.1, NP_{—}744048.1, YP_{—}258889.1, YP_{—}004474743.1, YP_{—}004380764.1, YP_{—}349912.1, YP_{—}004700980.1, YP_{—}001269130.1, ADR61321.1, YP_{—}001347709.1, YP_{—}004355482.1, NP_{—}251505.1, ZP_{—}04929120.1, YP_{—}002871500.1, EGM21899.1, YP_{—}001187411.1, ZP_{—}07794119.1, ZP_{—}07774414.1, YP_{—}001172167.1, YP_{—}004713921.1, ZP_{—}08138366.1, AEA83572.1, YP_{—}001667709.1, YP_{—}607261.1, YP_{—}001748377.1, YP_{—}260045.1, YP_{—}002873091.1, ZP_{—}07775826.1, CAC34855.1, EGH11916.1, ZP_{—}05641615.1, ZP_{—}06480669.1, ZP_{—}06480668.1, ZP_{—}05641616.1, ZP_{—}06492823.1, ZP_{—}06492821.1, EGH11920.1, EGH25319.1, ZP_{—}06492824.1, ADX52254.1, AP_{—}000876.1, ZP_{—}08341828.1, YP_{—}002291517.1, YP_{—}003227327.1, YP_{—}001461409.1, AEG35025.1, YP_{—}002385739.1, ZP_{—}08352177.1, ZP_{—}03070250.1, ZP_{—}08367389.1, CBI99746.1, ZP_{—}06660773.1, ZP_{—}08372569.1, YP_{—}003497883.1, ACI71032.1, YP_{—}002406464.1, EGB79412.1, EFZ76765.1, ZP_{—}07145000.1, ZP_{—}07151031.1, EFZ56676.1, ZP_{—}06656148.1, EGB35012.1, EGB71054.1, ZP_{—}07183316.1, YP_{—}002396328.1, YP_{—}002327805.1, ZP_{—}03027602.1, AAG54546.1, YP_{—}001742341.1, ABE05764.1, EFX12717.1, ACI71029.1, NP_{—}285938.2, ZP_{—}07120505.1, YP_{—}539295.2, ZP_{—}03049609.1, ZP_{—}06652178.1, ABI99723.1, EGB60663.1, EFW71911.1, EGH38574.1, YP_{—}668186.1, ZP_{—}02801146.2, ZP_{—}08346491.1, ZP_{—}08362542.1, AAN78852.1, NP_{—}752308.2, ZP_{—}07173894.1, ZP_{—}08357265.1, ZP_{—}08382276.1, AAM28523.1, ZP_{—}07097521.1, EGB41427.1, EGB41426.1, BAA07583.1, ZP_{—}07100038.1, CAX20347.1
and particularly preferably
NP_{—}744048.1, YP_{—}004700980.1, YP_{—}001269130.1, ADR61321.1, YP_{—}001667709.1, YP_{—}001748377.1, YP_{—}258889.1, YP_{—}349912.1, YP_{—}002871500.1, ZP_{—}07774414.1, YP_{—}260045.1, YP_{—}002873091.1, ZP_{—}07775826.1, CAC34855.1, YP_{—}001172167.1, YP_{—}004713921.1, AEA83572.1, AP_{—}000876.1, BAA07583.1, ZP_{—}07594808.1.
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E_{b} in general is understood to mean in particular the oxidation of dodecanoyl-CoA thioesters to 2-dodecenoyl-CoA thioesters.

### Specific Enzymes E_{c}

It is also preferable according to the invention that in the cells according to the invention the enzyme E_{c} is one which comprises sequences selected from:

YP_{—}003571780.1, YP_{—}445820.1, YP_{—}634556.1, YP_{—}004665862.1, ZP_{—}01461690.1, YP_{—}921666.1, YP_{—}002778910.1, ZP_{—}08550394.1, YP_{—}003384289.1, YP_{—}001195727.1, YP_{—}702012.1, ZP_{—}04384437.1, YP_{—}002765110.1, ZP_{—}04996322.1, ZP_{—}08195144.1, ZP_{—}04700546.1, YP_{—}954595.1, YP_{—}004736804.1, ADW07059.1, YP_{—}001827916.1, ZP_{—}04691466.1, YP_{—}001109453.1, ZP_{—}08240125.1, YP_{—}003272226.1, YP_{—}004053469.1, ZP_{—}06272176.1, YP_{—}004491616.1, YP_{—}001133991.1, YP_{—}001071715.1, YP_{—}290295.1, YP_{—}003193744.1, YP_{—}001704317.1, YP_{—}004008413.1, YP_{—}004655806.1, YP_{—}640598.1, ZP_{—}08153802.1, ZP_{—}00995173.1, ZP_{—}05225674.1, YP_{—}888747.1, YP_{—}003114111.1, YP_{—}004522832.1, ZP_{—}06848773.1, ZP_{—}08203814.1, YP_{—}001851901.1, EGO40578.1, YP_{—}003134974.1, ZP_{—}07282448.1, YP_{—}003770185.1, YP_{—}881295.1, YP_{—}004336131.1, NP_{—}961035.1, YP_{—}004164861.1, YP_{—}003681133.1, ZP_{—}04749633.1, ZP_{—}07718288.1, ZP_{—}01201898.1, YP_{—}004223976.1, YP_{—}118690.1, YP_{—}905275.1, BAE47462.1, YP_{—}831622.1, YP_{—}003407476.1, ZP_{—}01129477.1, YP_{—}003645654.1, YP_{—}004454693.1, YP_{—}002487953.1, YP_{—}004084231.1, YP_{—}003836912.1, YP_{—}004241154.1, ZP_{—}07706098.1, YP_{—}001855531.1, ZP_{—}08124588.1, YP_{—}947882.1, BAE47461.1, YP_{—}003327670.1, YP_{—}001363757.1, YP_{—}004601796.1, YP_{—}001625220.1, YP_{—}003638017.1, ZP_{—}06501585.1, YP_{—}004404736.1, YP_{—}062974.1, YP_{—}002957230.1, YP_{—}003316209.1, YP_{—}003149881.1, YP_{—}001221553.1, YP_{—}003162313.1, ZP_{—}03978917.1, YP_{—}001708860.1, ZP_{—}05912043.1, ZP_{—}06806059.1, YP_{—}003155732.1, YP_{—}002835700.1, YP_{—}003916799.1, ZP_{—}03936415.1, ZP_{—}07090640.1, ZP_{—}08516453.1, AAB97825.1, YP_{—}004541029.1, YP_{—}004606508.1, YP_{—}001801238.1, ZP_{—}07989876.1, YP_{—}004761186.1, YP_{—}002883572.1, ZP_{—}08023616.1, ZP_{—}05847263.1, YP_{—}251740.1, ZP_{—}03394212.1, YP_{—}001107648.1, YP_{—}002872770.1, YP_{—}001821654.1, ZP_{—}08233739.1, AAD12170.1, ZP_{—}08215859.1, AAD40800.1, ZP_{—}05005905.1, ADW07311.1, YP_{—}348592.1, NP_{—}824883.1, NP_{—}627459.1, YP_{—}001828149.1, ZP_{—}05525554.1, ZP_{—}08240364.1, ZP_{—}07299658.1, ZP_{—}06582153.1, ZP_{—}06921827.1, ZP_{—}04703961.1, BAJ27090.1, ZP_{—}06592678.1, ZP_{—}04691265.1, YP_{—}001751500.1, BAJ31579.1, preferably YP_{—}003571780.1, YP_{—}445820.1, YP_{—}634556.1, YP_{—}004665862.1, ZP_{—}01461690.1, YP_{—}921666.1, YP_{—}002778910.1, ZP_{—}08550394.1, YP_{—}003384289.1, YP_{—}001195727.1, YP_{—}702012.1, ZP_{—}04384437.1, YP_{—}002765110.1, ZP_{—}04996322.1, ZP_{—}08195144.1, ZP_{—}04700546.1, YP_{—}954595.1, YP_{—}004736804.1, ADW07059.1, YP_{—}001827916.1, ZP_{—}04691466.1, YP_{—}001109453.1, ZP_{—}08240125.1, YP_{—}003272226.1, YP_{—}004053469.1, ZP_{—}06272176.1, YP_{—}004491616.1, YP_{—}001133991.1, YP_{—}001071715.1, YP_{—}290295.1, YP_{—}003193744.1, YP_{—}001704317.1, YP_{—}004008413.1, YP_{—}004655806.1, YP_{—}640598.1, ZP_{—}08153802.1, ZP_{—}00995173.1, ZP_{—}05225674.1, YP_{—}888747.1, YP_{—}003114111.1, YP_{—}004522832.1, ZP_{—}06848773.1, ZP_{—}08203814.1, YP_{—}001851901.1, EGO40578.1, YP_{—}003134974.1, ZP_{—}07282448.1, YP_{—}003770185.1, YP_{—}881295.1, YP_{—}004336131.1, NP_{—}961035.1, YP_{—}004164861.1, YP_{—}003681133.1, ZP_{—}04749633.1, ZP_{—}07718288.1, ZP_{—}01201898.1, YP_{—}004223976.1, YP_{—}118690.1, YP_{—}905275.1, BAE47462.1, YP_{—}831622.1, YP_{—}003407476.1, ZP_{—}01129477.1, YP_{—}003645654.1, YP_{—}004454693.1, YP_{—}002487953.1, YP_{—}004084231.1, YP_{—}003836912.1, YP_{—}004241154.1, ZP_{—}07706098.1, YP_{—}001855531.1, ZP_{—}08124588.1, YP_{—}947882.1, BAE47461.1, YP_{—}003327670.1, YP_{—}001363757.1, YP_{—}004601796.1, YP_{—}001625220.1, YP_{—}003638017.1, ZP_{—}06501585.1, YP_{—}004404736.1, YP_{—}062974.1, YP_{—}002957230.1, YP_{—}003316209.1, YP_{—}003149881.1, YP_{—}001221553.1, YP_{—}003162313.1, ZP_{—}03978917.1, YP_{—}001708860.1, ZP_{—}05912043.1, ZP_{—}06806059.1, YP_{—}003155732.1, YP_{—}002835700.1, YP_{—}003916799.1, ZP_{—}03936415.1, ZP_{—}07090640.1, ZP_{—}08516453.1, AAB97825.1, YP_{—}004541029.1, YP_{—}004606508.1, YP_{—}001801238.1, ZP_{—}07989876.1, YP_{—}004761186.1, YP_{—}002883572.1, ZP_{—}08023616.1, ZP_{—}05847263.1, YP_{—}251740.1,
and particularly preferably YP_{—}002835700.1, ZP_{—}03936415.1, BAE47461.1, YP_{—}001801238.1, ZP_{—}03978917.1, ZP_{—}03394212.1, ZP_{—}05847263.1, ZP_{—}08516453.1, YP_{—}004606508.1, YP_{—}251740.1, ZP_{—}07090640.1, ZP_{—}07989876.1, YP_{—}004761186.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E_{c} in general is understood to mean in particular the oxidation of dodecanoyl-CoA thioesters to 2-dodecenoyl-CoA thioesters.

### Specific Enzymes E_{d} and Eₑ

It is also preferable according to the invention that in the cells according to the invention the enzyme E_{d} or Eₑ is one which comprises sequences selected from:

ZP_{—}07164313.1, NP_{—}418288.1, YP_{—}003231641.1, EGM59778.1, EFZ53307.1, AAA23750.1, ZP_{—}07192215.1, YP_{—}001460638.1, YP_{—}001727088.1, EGK16564.1, ZP_{—}08380619.1, ZP_{—}07136310.1, CAB40809.1, NP_{—}839030.1, ZP_{—}07690617.1, EGC97039.1, ZP_{—}07103516.1, ZP_{—}03027888.1, ZP_{—}07121980.1, YP_{—}002414996.1, EGP22873.1, EGJ82677.1, EGB59499.1, ZP_{—}07118761.1, YP_{—}002409078.1, YP_{—}002295407.1, EGE62412.1, EGB69560.1, ZP_{—}06655948.1, ZP_{—}06664574.1, ZP_{—}03070699.1, ZP_{—}07145404.1, ZP_{—}08376058.1, EGB85466.1, ZP_{—}07189176.1, ZP_{—}02999920.1, ZP_{—}08356523.1, ZP_{—}06659936.1, ZP_{—}07139396.1, YP_{—}001746178.1, YP_{—}002384700.1, ZP_{—}07098889.1, CBG37051.1, ZP_{—}04873109.1, CBJ03626.1, ZP_{—}08366395.1, ZP_{—}03066301.1, BAI57243.1, YP_{—}001465330.1, YP_{—}405325.1, NP_{—}312801.1, EGI89589.1, EGC09628.1, EFW73050.1, ZP_{—}07221474.1, EGB39932.1, EFW72281.1, ZP_{—}07154547.1, YP_{—}002331616.1, EGB76756.1, EFZ75005.1, ZP_{—}07449248.1, NP_{—}756652.2, ZP_{—}04006347.1, NP_{—}290476.1, EGH36687.1, YP_{—}671920.1, ZP_{—}08350773.1, EGC05062.1, ZP_{—}07174622.1, CAP78309.1, ZP_{—}08361145.1, YP_{—}002400350.1, ZP_{—}08386169.1, EFU60028.1, ZP_{—}02904283.1, YP_{—}859447.1, YP_{—}543379.2, NP_{—}462868.1, ZP_{—}02663494.1, ACY91152.1, ZP_{—}03221347.1, YP_{—}001591071.1, YP_{—}002639596.1, EFY10009.1, ZP_{—}04656823.1, ZP_{—}03213459.1, ZP_{—}02701437.1, ZP_{—}02347126.1, YP_{—}002217909.1, ZP_{—}02658976.1, YP_{—}002245833.1, YP_{—}002228260.1, YP_{—}001451793.1, EGE35935.1, YP_{—}002047992.1, ZP_{—}02834645.1, ZP_{—}02669606.1, YP_{—}001572623.1, ZP_{—}03075319.1, YP_{—}002148908.1, YP_{—}004591813.1, ZP_{—}03163685.1, YP_{—}002043211.1, NP_{—}457769.1, YP_{—}003367347.1, YP_{—}004732313.1, ZP_{—}06546517.1, ZP_{—}08495782.1, ZP_{—}04558441.1, YP_{—}002241091.1, ZP_{—}06354348.2, ZP_{—}06552493.1, YP_{—}001337975.1, YP_{—}001178655.1, CBK87780.1, ZP_{—}05970964.1, ZP_{—}06014071.1, YP_{—}002917176.1, YP_{—}152910.1, Q9F0Y7.1, ZP_{—}08302760.1, YP_{—}003615422.1, YP_{—}003943709.1, EGI93642.1, YP_{—}001439747.1, YP_{—}003208640.1, YP_{—}001476499.1, ZP_{—}06638309.1, YP_{—}004498688.1, YP_{—}004296470.1, ZP_{—}06192594.1, YP_{—}001004653.1, ZP_{—}04634366.1, CBY29055.1, ZP_{—}04641538.1, ZP_{—}04628383.1, ZP_{—}04620754.1, ZP_{—}04624649.1, YP_{—}003739635.1, ZP_{—}07953302.1, YP_{—}001399280.1, NP_{—}667802.1, YP_{—}068813.1, ADV97208.1, ZP_{—}04637125.1, YP_{—}003019595.1, YP_{—}048335.1, ZP_{—}04612255.1, ZP_{—}03831342.1, ZP_{—}03827989.1, YP_{—}003261565.1, ZP_{—}04616540.1, EFW54755.1, YP_{—}004214864.1, BAK13441.1, YP_{—}003518496.1, YP_{—}003933023.1, ZP_{—}07380063.1, YP_{—}003042702.1, YP_{—}003713991.1, YP_{—}003466462.1, YP_{—}004114076.1, YP_{—}001906200.1, NP_{—}931575.1, EGK17810.1, CBX79037.1, YP_{—}003529581.1, ZP_{—}06937250.1, YP_{—}002647270.1, ADP11112.1, ZP_{—}05974166.1, ZP_{—}03318464.1, ZP_{—}02958886.1, YP_{—}003331802.1, ZP_{—}06125606.1, YP_{—}003006180.1, YP_{—}003885045.1, YP_{—}128321.1, ZP_{—}01236908.1, ZP_{—}01161145.1, YP_{—}002989323.1, YP_{—}002154796.1, YP_{—}203408.1, ZP_{—}08310903.1, YP_{—}002264299.1, ZP_{—}01221704.1, ZP_{—}06050960.1, ZP_{—}03841335.1, ZP_{—}05883431.1, YP_{—}002153226.1, ADT85583.1, ZP_{—}05879947.1, ZP_{—}04923724.1, ZP_{—}01262258.1, ZP_{—}06179383.1, ZP_{—}05883853.1, EGF42158.1, ZP_{—}01957954.1, ZP_{—}08101926.1, ZP_{—}06177050.1, NP_{—}759944.1, NP_{—}796409.1, ZP_{—}04419618.1, ZP_{—}01987794.1, ZP_{—}05121182.1, YP_{—}001443702.1, ZP_{—}01948571.1, ZP_{—}01682057.1, ZP_{—}04405432.1, NP_{—}232384.1, ZP_{—}04409574.1, ZP_{—}01870127.1, NP_{—}932822.1, ZP_{—}06943917.1, EGR05147.1, ZP_{—}04961951.1, EGR10674.1, ZP_{—}04414292.1, ZP_{—}05718020.1, ZP_{—}08098153.1, ZP_{—}05719938.1, ZP_{—}03356468.1, ZP_{—}07742015.1, YP_{—}004564872.1, ZP_{—}01979859.1, ZP_{—}00992843.1, ZP_{—}05927571.1, ZP_{—}01065523.1, YP_{—}002415749.1, ZP_{—}01815881.1, ZP_{—}02196043.1, YP_{—}001143922.1, ZP_{—}08518445.1, ZP_{—}06156529.1, YP_{—}004394586.1, ZP_{—}01900693.1, YP_{—}854676.1, ZP_{—}05943242.1, CBA71812.1, ZP_{—}01991723.1, YP_{—}001092151.1, YP_{—}001672251.1, YP_{—}961420.1, YP_{—}003554801.1, YP_{—}003911300.1, ADV52504.1, YP_{—}001364252.1, YP_{—}001552462.1, ZP_{—}07394327.1, YP_{—}001048426.1, YP_{—}002355987.1, ZP_{—}02158912.1, ABE53312.1, YP_{—}561035.2, YP_{—}748714.1, ZP_{—}01135242.1, NP_{—}715663.1, YP_{—}732157.1, YP_{—}867675.1, YP_{—}736079.1, YP_{—}001758417.1, YP_{—}001499882.1, YP_{—}004436299.1, YP_{—}659787.1, ZP_{—}08620874.1, YP_{—}002309470.1, CBW44433.1, ZP_{—}08568624.1, YP_{—}958423.1, YP_{—}925914.1, YP_{—}001471764.1, ZP_{—}01165107.1, ZP_{—}04717156.1, ZP_{—}01042072.1, ZP_{—}08568929.1, YP_{—}004468425.1, ZP_{—}01614054.1, EGH60623.1, NP_{—}744285.1, ZP_{—}04587907.1, EGH84450.1, YP_{—}609235.1, Q93Q12.1, ZP_{—}07263341.1, YP_{—}004425808.1, EGH10831.1, ZP_{—}08142928.1, YP_{—}435877.1, YP_{—}004701152.1, ADR61111.1, EGH72107.1, ZP_{—}07255969.1, EGH76237.1, YP_{—}154404.1, EGH66371.1, ZP_{—}07005687.1, YP_{—}001268914.1, ZP_{—}03397164.1, YP_{—}267151.1, EGH45982.1, NP_{—}793297.1, YP_{—}236360.1, YP_{—}001667915.1, EGH29726.1, ZP_{—}03561781.1, YP_{—}275370.1, ABP88736.1, ZP_{—}06458302.1, YP_{—}001748526.1, YP_{—}002871195.1, ZP_{—}06478839.1, EGH95845.1, YP_{—}004067126.1, EGH21541.1, ZP_{—}05638744.1, Q9AHY3.2, YP_{—}338568.1, ZP_{—}06078672.1, YP_{—}004352961.1, ZP_{—}01892768.1, ZP_{—}06040413.1, YP_{—}349607.1, YP_{—}259059.1, ZP_{—}08409548.1, ADP97276.1, YP_{—}004713990.1, YP_{—}003626258.1, P28793.1, YP_{—}001172246.1, YP_{—}003810247.1, YP_{—}004313957.1, EGE21928.1, EGE19309.1, EGE13641.1, ZP_{—}08462037.1, EGE13529.1, ZP_{—}06034789.1, EGE12165.1, AEA83639.1, YP_{—}002798635.1, ZP_{—}01306165.1, YP_{—}004474976.1, ZP_{—}01739261.1, NP_{—}251704.1, ACP17923.1, YP_{—}004379416.1, YP_{—}001280990.1, YP_{—}003145204.1, YP_{—}001347517.1, ZP_{—}06877966.1, YP_{—}001187076.1, ZP_{—}08638729.1, YP_{—}001340441.1, ZP_{—}05128804.1, YP_{—}003896827.1, YP_{—}003073151.1, ZP_{—}05096745.1, ZP_{—}01103278.1, YP_{—}693372.1, ZP_{—}01366482.1, ZP_{—}05619303.1, ZP_{—}08328596.1, ZP_{—}05042935.1, YP_{—}574439.1, ZP_{—}01074264.1, YP_{—}004482149.1, YP_{—}045111.1, YP_{—}265216.1, ZP_{—}05362445.1, YP_{—}001715228.1, YP_{—}001844981.1, YP_{—}001708314.1, YP_{—}581488.1, ADY83798.1, ZP_{—}06692406.1, YP_{—}003733838.1, ZP_{—}05824704.1, ZP_{—}06058514.1, ZP_{—}08554004.1, ZP_{—}06068411.1, ZP_{—}06067277.1, ZP_{—}06726497.1, ADX01983.1, ZP_{—}03822268.1, ZP_{—}03347927.1, ZP_{—}01116792.1, YP_{—}527079.1, ZP_{—}06063435.1, ZP_{—}06534677.1, ZP_{—}01219812.1, ZP_{—}03347768.1, YP_{—}002798829.1, ZP_{—}07774142.1, YP_{—}003557881.1, ZP_{—}06157092.1, ZP_{—}01223872.1, ZP_{—}05946076.1, ZP_{—}06499586.1, YP_{—}003451185.1, YP_{—}002361722.1, YP_{—}003266103.1, YP_{—}285556.2, AAZ47086.1, NP_{—}968701.1, ZP_{—}06936670.1, ZP_{—}03805048.1, YP_{—}943922.1, ZP_{—}01217009.1, ADT87675.1, ZP_{—}05877956.1, ZP_{—}03355309.1, ZP_{—}05885304.1, EGK17811.1, ZP_{—}05944972.1,
ZP_{—}05119053.1, ZP_{—}06039619.1, ZP_{—}05716842.1, ZP_{—}05721090.1, ZP_{—}06079171.1, ZP_{—}06033023.1, ZP_{—}08098475.1, ZP_{—}08104504.1, ZP_{—}06048048.1, ZP_{—}01677170.1, ZP_{—}01681193.1, NP_{—}230692.2, ZP_{—}05926205.1, ZP_{—}05881372.1, ZP_{—}01975051.1, ZP_{—}04412573.1, ZP_{—}01977591.1, ZP_{—}04415061.1, ZP_{—}06048243.1, YP_{—}742943.1, ZP_{—}04962518.1, ZP_{—}01955504.1, ZP_{—}07741831.1, EGK33112.1, ZP_{—}01980800.1, CBW26643.1, EGQ99075.1, ZP_{—}03561616.1, ZP_{—}06155835.1, ZP_{—}01613403.1, YP_{—}003147156.1, ZP_{—}01866421.1, ZP_{—}08569601.1, YP_{—}004068133.1, ZP_{—}01992793.1, YP_{—}003760621.1, NP_{—}760849.1, NP_{—}935233.1, YP_{—}661240.1, CBA76402.1, YP_{—}003527567.1, ZP_{—}05071916.1, YP_{—}155382.1, ZP_{—}08567109.1, ZP_{—}08410490.1, YP_{—}002357526.1, YP_{—}001473368.1, ZP_{—}05061211.1, ZP_{—}08309062.1, ZP_{—}00990722.1, ZP_{—}01813160.1, YP_{—}343735.1, YP_{—}001366977.1, ZP_{—}07393465.1, YP_{—}002312436.1, ZP_{—}03805047.1, ZP_{—}04716066.1, ZP_{—}01043968.1, YP_{—}562538.1, ZP_{—}01064421.1, YP_{—}928042.1, YP_{—}002416486.1, YP_{—}962941.1, YP_{—}001051116.1, YP_{—}004467793.1, YP_{—}004434876.1, YP_{—}001183979.1, ZP_{—}01125518.1, YP_{—}001555281.1, ZP_{—}01900341.1, YP_{—}001459147.1, ADV54930.1, ZP_{—}06054161.1, YP_{—}001674882.1, YP_{—}001381324.1, ZP_{—}02158374.1, NP_{—}718651.1, YP_{—}737529.1, YP_{—}869101.1, ZP_{—}01258852.1, ZP_{—}05978956.1, ZP_{—}06179776.1, YP_{—}733543.1, ZP_{—}01989664.1, NP_{—}798587.1, EGF45285.1, ZP_{—}05908370.1, YP_{—}001502453.1, ZP_{—}06639387.1, YP_{—}003557654.1, ZP_{—}04921889.1, YP_{—}001436988.1, YP_{—}003468880.1, YP_{—}001761392.1, YP_{—}003267851.1, YP_{—}004730996.1, EGL72460.1, YP_{—}003742516.1, YP_{—}003258850.1, ZP_{—}01132697.1, ZP_{—}01987078.1, YP_{—}004392689.1, ZP_{—}06191156.1, YP_{—}002381996.1, ZP_{—}06176023.1, EGC06853.1, ZP_{—}07196084.1, NP_{—}754768.1, ZP_{—}02901855.1, ZP_{—}08620438.1, EGE30558.1, YP_{—}003211325.1, ZP_{—}03220131.1, YP_{—}217377.1, YP_{—}003940937.1, YP_{—}004669896.1, YP_{—}633521.1, YP_{—}002041652.1, NP_{—}456929.1, YP_{—}001446296.1, ZP_{—}02699767.1, YP_{—}001586838.1, YP_{—}751355.1, ZP_{—}08384609.1, YP_{—}002216460.1, A8GH86.2, ZP_{—}02667448.1, YP_{—}004595105.1, YP_{—}002408448.1, YP_{—}001479604.1, YP_{—}149790.1, NP_{—}461330.1, YP_{—}002227302.1, ZP_{—}07187886.1, ZP_{—}08374604.1, ZP_{—}02343362.1, ZP_{—}02683558.1, YP_{—}001141958.1, ZP_{—}02662473.1, ZP_{—}07151809.1, YP_{—}004211957.1, YP_{—}003366276.1, YP_{—}003713364.1, ZP_{—}03035287.1, ZP_{—}08364768.1, YP_{—}002413389.1, ZP_{—}07448710.1, ZP_{—}04656170.1, ZP_{—}02654823.1, ZP_{—}01222785.1, EGB63194.1, ZP_{—}08359459.1, YP_{—}002636921.1, YP_{—}002329984.1, YP_{—}001744544.1, CAP76837.1, EFZ73229.1, EFU57443.1, YP_{—}002398712.1, YP_{—}003018387.1, ZP_{—}08520753.1, YP_{—}541623.1, ZP_{—}02574174.1, ZP_{—}07144040.1, ZP_{—}08349090.1, CBG35413.1, ZP_{—}04562847.1, ZP_{—}02195785.1, ZP_{—}02773221.1, EGB40918.1, ZP_{—}03050715.1, ZP_{—}07787570.1, ZP_{—}03831301.1, YP_{—}003003682.1, ZP_{—}08354786.1, YP_{—}051168.1, YP_{—}002403607.1, AEE57458.1, YP_{—}856678.1, YP_{—}001177597.1, ZP_{—}06658276.1, NP_{—}288914.1, YP_{—}002392166.1, ZP_{—}06654274.1, ZP_{—}07102361.1, EGB72544.1, YP_{—}004501987.1, ZP_{—}03027319.1, YP_{—}670274.1, YP_{—}003913906.1, ZP_{—}07097669.1, YP_{—}001463687.1, BAI55757.1, ZP_{—}08553509.1, YP_{—}003500399.1, ZP_{—}07121648.1, ZP_{—}01235780.1, CBK87125.1, YP_{—}002293925.1, ZP_{—}05431367.1, YP_{—}129175.1, ZP_{—}03003629.1, YP_{—}002387809.1, ZP_{—}03043524.1, YP_{—}001569579.1, ZP_{—}05435840.1, ZP_{—}01464666.1, YP_{—}001724305.1, ZP_{—}03068335.1, CBJ01980.1, AEJ57562.1, NP_{—}416843.1, YP_{—}002920590.1, ZP_{—}03828462.1, EGM60943.1, ZP_{—}06351976.1, ZP_{—}05968584.1, EGK21055.1, YP_{—}003040254.1, NP_{—}708223.1, YP_{—}689824.1, ZP_{—}04625886.1, AEJ99232.1, ZP_{—}07135079.1, YP_{—}339488.1, ZP_{—}07247352.1, ZP_{—}07590743.1, ZP_{—}08303100.1, EFU96242.1, EFZ69715.1, YP_{—}001336370.1, YP_{—}001094550.1, ZP_{—}07679578.1, ZP_{—}06547779.1, EGI93593.1, YP_{—}003438264.1, YP_{—}003614165.1, YP_{—}408769.1, YP_{—}001881164.1, YP_{—}003655512.1, YP_{—}002237269.1, YP_{—}004116642.1, ZP_{—}03065203.1, ZP_{—}07951118.1, CAQ79951.1, AAZ26206.1, BAK12062.1, YP_{—}269853.2, NP_{—}930429.2, YP_{—}404102.1, ZP_{—}04620204.1, ZP_{—}08498986.1, YP_{—}001452041.1, ZP_{—}01159981.1, CAE15574.1, A1JK30.2, ZP_{—}04635573.1, ZP_{—}02904987.1, ZP_{—}02961182.1, YP_{—}001005598.1, ZP_{—}01301762.1, ZP_{—}06016509.1, CBY28037.1, ZP_{—}05060968.1, ZP_{—}04632512.1, YP_{—}002156637.1, YP_{—}002132807.1, Q5E3U1.2, YP_{—}205193.1, ZP_{—}04613435.1, ZP_{—}07380136.1, YP_{—}004299028.1, YP_{—}003334344.1, YP_{—}001610684.1, YP_{—}001720255.1, YP_{—}001400379.1, YP_{—}652007.1, NP_{—}668898.1, ZP_{—}04640314.1, ADV98116.1, ZP_{—}03840558.1, ZP_{—}07047543.1, ZP_{—}03320348.1, YP_{—}001681761.1, ZP_{—}04615169.1, ZP_{—}08182604.1, YP_{—}003520988.1, YP_{—}002151536.1, NP_{—}641653.1, ZP_{—}08188276.1, Q668V1.2, YP_{—}463621.1, ZP_{—}05032523.1, YP_{—}363100.1, YP_{—}002490860.1, YP_{—}071146.1, YP_{—}003527951.1, YP_{—}004615064.1, ZP_{—}06702935.1, YP_{—}003277339.1, ZP_{—}06729873.1, YP_{—}004552309.1, ZP_{—}08178119.1, YP_{—}558747.1, YP_{—}003059322.1, ZP_{—}04628689.1, ZP_{—}05043496.1, YP_{—}755774.1, NP_{—}106254.1, NP_{—}774461.1, YP_{—}004145058.1, NP_{—}636640.1, YP_{—}001411745.1 YP_{—}244043.1, YP_{—}003906899.1, ZP_{—}02151779.1, EFW54754.1, YP_{—}004147062.1, YP_{—}434583.1, ZP_{—}06862658.1, YP_{—}003559491.1, ZP_{—}07474361.1, ZP_{—}07478578.1, ZP_{—}03787298.1, ZP_{—}06840682.1, ZP_{—}05161835.1, ZP_{—}06794105.1, ZP_{—}05181908.1, ZP_{—}05174379.1, YP_{—}003883888.1, NP_{—}541475.1, NP_{—}949054.1, YP_{—}003931777.1, YP_{—}001993209.1, ZP_{—}06124668.1, YP_{—}001594738.1, ZP_{—}06070710.1, ZP_{—}06484372.1, YP_{—}002515449.1, YP_{—}001895558.1, YP_{—}002029364.1, ZP_{—}02891585.1, ZP_{—}04682672.1, YP_{—}003761433.1, YP_{—}004107983.1, YP_{—}223224.1, YP_{—}003812264.1, YP_{—}001622574.1, ZP_{—}05452320.1, YP_{—}002734532.1, YP_{—}001257739.1, YP_{—}001372564.1, ZP_{—}05137372.1, YP_{—}001973266.1, YP_{—}342869.1, NP_{—}699967.1, ZP_{—}05086267.1, ZP_{—}01736760.1, YP_{—}001914218.1, ZP_{—}05157647.1, YP_{—}485365.1, YP_{—}001926123.1, ZP_{—}05116437.1, ZP_{—}03544469.1, ZP_{—}08330383.1, ZP_{—}06491403.1, ZP_{—}01896167.1, ADP99705.1, ZP_{—}02883593.1, YP_{—}004228182.1, YP_{—}570677.1, ZP_{—}01225298.1, YP_{—}200487.1, YP_{—}002988196.1, ZP_{—}08269313.1, NP_{—}767800.1, YP_{—}001094989.1, ZP_{—}06065014.1, YP_{—}002981447.1, YP_{—}001260831.1, YP_{—}003817548.1, YP_{—}532099.1, ZP_{—}07676723.1, YP_{—}001242863.1, ZP_{—}02244047.1, YP_{—}982073.1, YP_{—}001899020.1, NP_{—}519880.1, ZP_{—}02379339.1, NP_{—}946171.1, ZP_{—}01615132.1, YP_{—}456953.1, ZP_{—}02168372.1, ZP_{—}08552434.1, CBJ37969.1, YP_{—}004418392.1, ZP_{—}02362492.1, YP_{—}004107339.1, YP_{—}001203133.1, ZP_{—}01546752.1, YP_{—}002974094.1, ZP_{—}02186892.1, YP_{—}001989920.1, YP_{—}002964466.1, ZP_{—}03265887.1, YP_{—}555553.1, CBA26305.1, ZP_{—}06728723.1, ZP_{—}07656835.1, ZP_{—}05620865.1, YP_{—}575713.1, YP_{—}001907090.1, YP_{—}002911224.1, YP_{—}047520.1, YP_{—}004688052.1,
in particular
EGH60623.1, NP_{—}744285.1, ZP_{—}04587907.1, EGH84450.1, YP_{—}609235.1, Q93Q12.1, ZP_{—}07263341.1, EGH10831.1, ZP_{—}08142928.1, YP_{—}004701152.1, ADR61111.1, EGH72107.1, ZP_{—}07255969.1, EGH76237.1, EGH66371.1, ZP_{—}07005687.1, YP_{—}001268914.1, ZP_{—}03397164.1, EGH45982.1, NP_{—}793297.1, YP_{—}236360.1, YP_{—}001667915.1, EGH29726.1, YP_{—}275370.1, ABP88736.1, ZP_{—}06458302.1, YP_{—}001748526.1, YP_{—}002871195.1, ZP_{—}06478839.1, EGH95845.1, EGH21541.1, ZP_{—}05638744.1, Q9AHY3.2, YP_{—}004352961.1, YP_{—}349607.1, YP_{—}259059.1, YP_{—}004713990.1, P28793.1, YP_{—}001172246.1, AEA83639.1, YP_{—}004474976.1, NP_{—}251704.1, ACP17923.1, YP_{—}004379416.1, YP_{—}001347517.1, ZP_{—}06877966.1, YP_{—}001187076.1, ZP_{—}01366482.1, ZP_{—}07774142.1, ZP_{—}06499586.1, YP_{—}791508.1, ZP_{—}07796310.1, NP_{—}250428.1, YP_{—}002441177.1, YP_{—}001348922.1, ZP_{—}06879352.1, AEA82038.1, YP_{—}001170648.1, YP_{—}004473370.1, YP_{—}004712521.1, YP_{—}004353314.1, ZP_{—}07164313.1, NP_{—}418288.1, YP_{—}003231641.1, AAA23750.1, ZP_{—}07192215.1, YP_{—}001460638.1, YP_{—}001727088.1, ZP_{—}08380619.1, ZP_{—}07136310.1, CAB40809.1, ZP_{—}07690617.1, ZP_{—}07103516.1, ZP_{—}03027888.1, ZP_{—}07121980.1, YP_{—}002414996.1, EGP22873.1, EGB59499.1, ZP_{—}07118761.1, YP_{—}002409078.1, YP_{—}002295407.1, EGE62412.1, EGB69560.1, ZP_{—}06655948.1, ZP_{—}06664574.1, ZP_{—}03070699.1, ZP_{—}07145404.1, ZP_{—}08376058.1, EGB85466.1, ZP_{—}07189176.1, ZP_{—}02999920.1, ZP_{—}08356523.1, ZP_{—}06659936.1, ZP_{—}07139396.1, YP_{—}001746178.1, ZP_{—}07098889.1, CBG37051.1, CBJ03626.1, ZP_{—}08366395.1, BAI57243.1, YP_{—}001465330.1, NP_{—}312801.1, EGC09628.1, EFW73050.1, ZP_{—}07221474.1, EGB39932.1, EFW72281.1, ZP_{—}07154547.1, YP_{—}002331616.1, EGB76756.1, EFZ75005.1, ZP_{—}07449248.1, NP_{—}756652.2, ZP_{—}04006347.1, NP_{—}290476.1, EGH36687.1, YP_{—}671920.1, ZP_{—}08350773.1, ZP_{—}07174622.1, CAP78309.1, ZP_{—}08361145.1, YP_{—}002400350.1, ZP_{—}08386169.1, EFU60028.1, YP_{—}859447.1, YP_{—}543379.2, ZP_{—}06937250.1, ZP_{—}06936670.1, YP_{—}001459147.1, ZP_{—}07196084.1, NP_{—}754768.1, ZP_{—}08384609.1, YP_{—}002408448.1, ZP_{—}07187886.1, ZP_{—}08374604.1, ZP_{—}07151809.1, ZP_{—}03035287.1, ZP_{—}08364768.1, YP_{—}002413389.1, ZP_{—}07448710.1, EGB63194.1, ZP_{—}08359459.1, YP_{—}002329984.1, YP_{—}001744544.1, CAP76837.1, EFZ73229.1, EFU57443.1, YP_{—}002398712.1, YP_{—}541623.1, ZP_{—}07144040.1, ZP_{—}08349090.1, CBG35413.1, ZP_{—}02773221.1, EGB40918.1, ZP_{—}03050715.1, ZP_{—}07787570.1, ZP_{—}08354786.1, YP_{—}002403607.1, AEE57458.1, ZP_{—}06658276.1, NP_{—}288914.1, YP_{—}002392166.1, ZP_{—}06654274.1, ZP_{—}07102361.1, EGB72544.1, ZP_{—}03027319.1, YP_{—}670274.1, ZP_{—}07097669.1, YP_{—}001463687.1, BAI55757.1, YP_{—}003500399.1, ZP_{—}07121648.1, YP_{—}002293925.1, ZP_{—}03003629.1, YP_{—}002387809.1, ZP_{—}03043524.1, YP_{—}001724305.1, ZP_{—}03068335.1, CBJ01980.1, AEJ57562.1, NP_{—}416843.1, ZP_{—}07135079.1, ZP_{—}07247352.1, ZP_{—}07590743.1, EFU96242.1, EFZ69715.1,
and particularly preferably
NP_{—}744285.1, YP_{—}004701152.1, ADR61111.1, YP_{—}001268914.1, YP_{—}001667915.1, ABP88736.1, YP_{—}001748526.1, Q9AHY3.2, YP_{—}004713990.1, YP_{—}001172246.1, AEA83639.1, AEA82038.1, YP_{—}001170648.1, YP_{—}004712521.1, YP_{—}002871195.1, YP_{—}349607.1, YP_{—}259059.1, ZP_{—}07774142.1, NP_{—}418288.1, NP_{—}416843.1, ZP_{—}07593201.1, ZP_{—}07590743.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E_{d} and Eₑ in general is understood to mean in particular the conversion of 2-dodecenoyl-CoA thioesters to 3-oxo-dodecanoyl-CoA thioesters.

### Specific Enzymes E_{f}

It is also preferable according to the invention that in the cells according to the invention the enzyme E_{f} is one which comprises sequences selected from:

YP_{—}026272.1, YP_{—}002389323.1, EGB30581.1, YP_{—}001460637.1, YP_{—}001727089.1, CAB40810.1, EGK16565.1, NP_{—}709649.1, YP_{—}001882545.1, ZP_{—}08356522.1, ZP_{—}06664573.1, AAA67642.1, ADA76222.1, EGK17812.1, YP_{—}405326.1, YP_{—}003236969.1, ZP_{—}06659935.1, YP_{—}410143.1, NP_{—}290475.1, ZP_{—}03027945.1, EFZ59092.1, YP_{—}002295406.1, CBG37050.1, EGP22872.1, EGE62411.1, EGC97040.1, ZP_{—}05435276.1, YP_{—}002400349.1, EGB59498.1, EFW54756.1, ZP_{—}08361144.1, YP_{—}001465329.1, YP_{—}002384701.1, YP_{—}002409079.1, ZP_{—}06655947.1, YP_{—}002414995.1, EGB69559.1, YP_{—}859446.1, EGC05061.1, ZP_{—}02904263.1, ZP_{—}08386168.1, YP_{—}543378.1, ZP_{—}08366394.1, ZP_{—}03066325.1, YP_{—}001746177.1, ZP_{—}07154548.1, ZP_{—}03070708.1, NP_{—}756651.1, YP_{—}312775.1, YP_{—}671919.1, YP_{—}002331615.1, YP_{—}003367348.1, ZP_{—}07449249.1, ZP_{—}04558440.1, ZP_{—}06354347.1, YP_{—}001451792.1, YP_{—}004732312.1, ZP_{—}06938722.1, NP_{—}457770.1, ZP_{—}02834646.1, ZP_{—}02658975.1, ZP_{—}03221210.1, EFY10008.1, YP_{—}001591070.1, YP_{—}218866.1, YP_{—}003943710.1, ZP_{—}03086141.1, ZP_{—}03163187.1, ZP_{—}08495783.1, EGE35936.1, YP_{—}003615423.1, YP_{—}002228261.1, YP_{—}002148907.1, ZP_{—}05970963.1, YP_{—}002241092.1, YP_{—}004591812.1, YP_{—}001178656.1, ZP_{—}08302761.1, YP_{—}002917175.1, YP_{—}001337974.1, Q9F0Y6.1, ZP_{—}06014072.1, YP_{—}001439748.1, YP_{—}003208639.1, 3GOA_A, YP_{—}003739634.1, ZP_{—}06192593.1, YP_{—}001906199.1, YP_{—}001476498.1, YP_{—}003019596.1, YP_{—}003261566.1, ZP_{—}03831341.1, ZP_{—}07380062.1, YP_{—}048334.1, YP_{—}003933022.1, ZP_{—}07953301.1, YP_{—}004114075.1, YP_{—}002647269.1, ADP11111.1, ZP_{—}03827988.1, ZP_{—}06638308.1, YP_{—}004214865.1, YP_{—}003518495.1, ZP_{—}04616541.1, BAK13440.1, CBX79036.1, YP_{—}003529580.1, ZP_{—}04628384.1, ZP_{—}04634365.1, ZP_{—}04641537.1, ZP_{—}04612254.1, ZP_{—}04637126.1, YP_{—}003331801.1, YP_{—}001004652.1, YP_{—}004296469.1, YP_{—}003006181.1, ZP_{—}04620755.1, YP_{—}003885046.1, ZP_{—}04624648.1, NP_{—}667801.1, EGI89588.1, YP_{—}128320.1, ZP_{—}01221705.1, YP_{—}002989324.1, ZP_{—}01236909.1, ZP_{—}01161146.1, ZP_{—}08310904.1, YP_{—}003713992.1, YP_{—}003466461.1, NP_{—}931576.1, YP_{—}003042703.1, ZP_{—}06050959.1, EGF42157.1, ZP_{—}00992844.1, YP_{—}002415748.1, ZP_{—}01065522.1, ZP_{—}05883432.1, ZP_{—}05879948.1, ZP_{—}06156528.1, ZP_{—}05927570.1, ZP_{—}07189177.1, YP_{—}004564871.1, ZP_{—}01870126.1, ZP_{—}02196042.1, YP_{—}003911299.1, ZP_{—}01815882.1, NP_{—}796408.1, YP_{—}004394587.1, ZP_{—}08518444.1, YP_{—}854675.1, ZP_{—}07742014.1, ZP_{—}01135243.1, YP_{—}002154795.1, NP_{—}759945.1, YP_{—}001143923.1, NP_{—}932821.1, Q5E8X7.2, ZP_{—}08568928.1, ZP_{—}06078671.1, ZP_{—}05718021.1, ZP_{—}01948567.1, YP_{—}203407.3, ZP_{—}04923725.1, ZP_{—}05719937.1, YP_{—}002309469.1, ZP_{—}06179384.1, ZP_{—}06048881.1, ZP_{—}01979851.1, ZP_{—}01262259.1, ZP_{—}01957951.1, ZP_{—}04405431.1, ZP_{—}08098152.1, ZP_{—}06034494.1, YP_{—}001758416.1, ZP_{—}04414293.1, ZP_{—}06040414.1, ZP_{—}01682043.1, NP_{—}232385.1, ZP_{—}05883854.1, YP_{—}002264298.1, ZP_{—}01987792.1, YP_{—}338567.1, ZP_{—}01900694.1, YP_{—}001672250.1, YP_{—}925913.1, YP_{—}001499881.1, ZP_{—}02158913.1, YP_{—}001471763.1, NP_{—}715662.1, YP_{—}748713.1, YP_{—}736078.1, ZP_{—}08568623.1, ZP_{—}02958885.2, YP_{—}004067125.1, ZP_{—}08409549.1, YP_{—}001181547.1, ADV52503.1, YP_{—}732156.1, YP_{—}001092150.1, YP_{—}003554800.1, YP_{—}001048425.1, YP_{—}961419.1, YP_{—}561034.1, ZP_{—}06125607.2, YP_{—}867674.1, YP_{—}001443701.1, ZP_{—}05943241.1, ZP_{—}05121169.1, ZP_{—}05974167.1, ZP_{—}03318463.1, ZP_{—}08620875.1, ZP_{—}01042073.1, YP_{—}154403.1, ZP_{—}04717155.1, ZP_{—}03805050.1, YP_{—}004468426.1, ZP_{—}03841336.1, YP_{—}002153225.1, YP_{—}004425807.1, ZP_{—}03351120.1, YP_{—}659788.1, YP_{—}004436298.1, YP_{—}267150.1, ZP_{—}06034790.1, YP_{—}003145205.1, ZP_{—}03561780.1, YP_{—}003810248.1, ZP_{—}05043383.1, YP_{—}693373.1, ZP_{—}01306166.1, YP_{—}004313956.1, YP_{—}790159.1, ZP_{—}06877967.1, NP_{—}251703.1, YP_{—}004482148.1, EGH66370.1, EGH10832.1, YP_{—}349606.1, YP_{—}236359.1, ZP_{—}07263340.1, EGH95844.1, ZP_{—}03368595.1, NP_{—}793296.1, ZP_{—}01165108.1, 1WDK_C, P28790.2, YP_{—}259060.1, ZP_{—}01074263.1, ZP_{—}04587908.1, EGH45981.1, ZP_{—}07774144.1, EGH84449.1, YP_{—}958424.1, YP_{—}275369.1, ZP_{—}07005686.1, YP_{—}001172247.1, YP_{—}004352962.1, ACP17922.1, YP_{—}002871196.1, YP_{—}435876.1, ZP_{—}01739262.1, YP_{—}003557880.1, ZP_{—}01892767.1, ZP_{—}08142929.1, ZP_{—}08462036.1, YP_{—}004701153.1, YP_{—}001667916.1, YP_{—}001280989.1, YP_{—}001268913.1, Q93Q11.1, ZP_{—}05619304.1, AEA79634.1, Q9R9W0.1, NP_{—}744286.1, YP_{—}001187077.1, YP_{—}609234.1, ADP97277.1, YP_{—}045110.1, YP_{—}004379417.1, YP_{—}003626259.1, ZP_{—}06692405.1, ZP_{—}06063436.1, YP_{—}003733839.1, EGE12166.1, ZP_{—}05824703.1, EGE26385.1, EGE13530.1, YP_{—}004474975.1, YP_{—}001708315.1, EGE16076.1, ZP_{—}06726496.1, ZP_{—}06067276.1, ZP_{—}06058513.1, ZP_{—}06068412.1, ZP_{—}06157093.1, ZP_{—}03822267.1, A3M1H9.2, YP_{—}001340442.1, ZP_{—}05362446.1, ABP88737.1, ZP_{—}01219813.1, ZP_{—}08638730.1, YP_{—}265215.1, YP_{—}581487.1, YP_{—}003896828.1, YP_{—}002798636.1, ZP_{—}01678475.1, ZP_{—}05946075.1, YP_{—}527080.1, ZP_{—}08554005.1, ZP_{—}03360083.1, YP_{—}574438.1, YP_{—}003073152.1, YP_{—}001083375.1, ZP_{—}08648989.1, YP_{—}001982171.1, ZP_{—}05096741.1, ZP_{—}03336985.1, ZP_{—}01103277.1, ZP_{—}07136312.1, ZP_{—}08328590.1, ZP_{—}05128805.1, EGH76239.1, ZP_{—}03377529.1, CBA71811.1, EFZ47010.1, ZP_{—}03377530.1, ZP_{—}07136311.1, EFZ47009.1, EGH29725.1, YP_{—}003022611.1, YP_{—}002138248.1, ZP_{—}01462439.1, ZP_{—}06499584.1, YP_{—}004669687.1, YP_{—}633289.1, ZP_{—}01907074.1, YP_{—}001611010.1, ADI22030.1, ZP_{—}03026937.1, YP_{—}580525.1, YP_{—}003265025.1, YP_{—}001525888.1, YP_{—}002298157.1, YP_{—}002945338.1, YP_{—}003271056.1, YP_{—}004198848.1, ZP_{—}01895445.1, ZP_{—}08636846.1, ADP95813.1, ZP_{—}03357270.1, YP_{—}002535575.1, YP_{—}160280.1, YP_{—}385012.1, YP_{—}004154467.1, YP_{—}742957.1, YP_{—}984918.1, ZP_{—}07949467.1, YP_{—}002552054.1, YP_{—}003439807.1, YP_{—}002919224.1, YP_{—}001475439.1, ZP_{—}07652842.1, YP_{—}001335140.1, YP_{—}972400.1, ZP_{—}08308052.1, YP_{—}001749490.1, YP_{—}004594280.1, ZP_{—}05360584.1, YP_{—}002490812.1, ZP_{—}03336986.1, YP_{—}004236457.1, ZP_{—}08387650.1, YP_{—}046370.1, ZP_{—}03823670.1, AEJ97944.1, ZP_{—}06188204.1, ABF82237.1, ZP_{—}06016043.1, YP_{—}046135.1, YP_{—}942111.1, ZP_{—}01614052.1, YP_{—}001341942.1, YP_{—}004713534.1, ZP_{—}01460231.1, YP_{—}001630800.1, YP_{—}001264278.1, CAD76924.1, ZP_{—}07200324.1, YP_{—}550745.1, YP_{—}001413963.1, YP_{—}002132758.1, ZP_{—}05972210.1, ZP_{—}06065848.1, ZP_{—}08209169.1, ZP_{—}06061642.1, ZP_{—}05109438.1, YP_{—}001419321.1, YP_{—}463572.1, YP_{—}608369.1, YP_{—}001683323.1, AEA83130.1, YP_{—}001230361.1, YP_{—}001832875.1, YP_{—}002237684.1, AAN39378.1, YP_{—}001019613.1, YP_{—}426398.1, ZP_{—}03543802.1, YP_{—}001171793.1, YP_{—}002138936.1, YP_{—}001562369.1, ZP_{—}01786296.1, YP_{—}001528043.1, NP_{—}881363.1, ZP_{—}04625099.1, ZP_{—}02187462.1, YP_{—}002355162.1, YP_{—}248479.1, ZP_{—}07043392.1, YP_{—}002028041.1, YP_{—}001900023.1, ZP_{—}01792340.1, ZP_{—}03541158.1, NP_{—}438930.1, ZP_{—}04464778.1, YP_{—}003278968.1, YP_{—}004490499.1, ZP_{—}07662038.1, AAM48101.1, YP_{—}422117.1, YP_{—}524752.1, YP_{—}918568.1, YP_{—}001264814.1, YP_{—}003807823.1, YP_{—}001260276.1, ZP_{—}07046088.1, ZP_{—}01784141.1, ZP_{—}05135853.1, YP_{—}002982015.1, EEZ80724.1, YP_{—}001292714.1, YP_{—}001971860.1, YP_{—}788379.1, ZP_{—}05783989.1, YP_{—}004415862.1, CAE45106.1, YP_{—}004618019.1, ZP_{—}01126529.1, ZP_{—}06062289.1, YP_{—}004538662.1, NP_{—}248919.1, YP_{—}001098905.1, YP_{—}003847633.1, YP_{—}002432816.1, YP_{—}003280245.1, A64092, ZP_{—}08404839.1, YP_{—}003466069.1, YP_{—}001348923.1, YP_{—}158582.1, YP_{—}004229600.1, ZP_{—}07797976.1, YP_{—}001416028.1, YP_{—}001747677.1, YP_{—}002362051.1, YP_{—}931973.1, ZP_{—}08505255.1, EGP53986.1, NP_{—}250427.1, YP_{—}366806.1, ZP_{—}04638299.1, ZP_{—}08485306.1, YP_{—}001675166.1, AAA23322.1, NP_{—}927515.1, AAR83740.1, YP_{—}433439.1, YP_{—}001668851.1, YP_{—}001713606.1, YP_{—}002354475.1, ZP_{—}06548530.1, ZP_{—}04764695.1, ZP_{—}01910282.1, YP_{—}004146469.1, YP_{—}095382.1, ZP_{—}06495825.1, YP_{—}003777379.1, ZP_{—}01914912.1, ZP_{—}06895226.1, YP_{—}004379898.1, YP_{—}003365234.1, YP_{—}001784146.1, YP_{—}003021900.1, YP_{—}004555586.1, YP_{—}001101071.1, CBW99592.1, YP_{—}003254723.1, AAG30258.1, YP_{—}004536011.1, NP_{—}884797.1, NP_{—}635761.1, YP_{—}002429235.1, YP_{—}001901798.1, ZP_{—}06485970.1, YP_{—}123631.1, YP_{—}001352245.1, ZP_{—}03697428.1, ZP_{—}05824476.1, ZP_{—}01014491.1, EGH60624.1, YP_{—}004028852.1, ZP_{—}04633718.1, YP_{—}001846659.1, ZP_{—}04933402.1, YP_{—}003731942.1, YP_{—}001345710.1, YP_{—}003979747.1, ZP_{—}00053266.1, YP_{—}126656.1, YP_{—}003442067.1, YP_{—}585810.1, ZP_{—}01614053.1, ZP_{—}06690229.1, YP_{—}001858908.1, ZP_{—}01128624.1, NP_{—}888558.1, ZP_{—}05827098.1, Q8VPF1.1, YP_{—}004473788.1, EGH77345.1, P45363.1, EGH44350.1, YP_{—}001676522.1, ZP_{—}05824514.1, ZP_{—}06487592.1, ZP_{—}02887415.1, ZP_{—}04761513.1, YP_{—}003377502.1, YP_{—}001188713.1, ZP_{—}01167911.1, ZP_{—}06690267.1, YP_{—}004680403.1, YP_{—}003731982.1, YP_{—}002800937.1, YP_{—}001758618.1, YP_{—}004380648.1, YP_{—}001188079.1, YP_{—}001707349.1, YP_{—}004687867.1, CAZ89607.1, ZP_{—}05827058.1, ZP_{—}08142248.1, YP_{—}195739.1, YP_{—}004703691.1, YP_{—}001354779.1, ZP_{—}08627639.1, ZP_{—}04936650.1, NP_{—}642338.1, ZP_{—}03451105.1, YP_{—}001713567.1, EFV87627.1, YP_{—}728366.1, YP_{—}002912837.1, YP_{—}001707333.1, YP_{—}363794.1, YP_{—}003524466.1, YP_{—}959751.1, YP_{—}606872.1, YP_{—}102034.1, YP_{—}002942733.1, YP_{—}002238110.1, ZP_{—}05032457.1, YP_{—}001846620.1, YP_{—}004153168.1, ZP_{—}02462362.1, YP_{—}003777513.1, YP_{—}199120.1, ZP_{—}08179077.1, ZP_{—}08188845.1, YP_{—}107279.1, ADP98459.1, YP_{—}004157409.1, YP_{—}610092.1, EGP55478.1, CBJ37328.1, ZP_{—}08181461.1, ZP_{—}06842278.1, ZP_{—}06703672.1, ADR61907.1, ZP_{—}06688595.1, ZP_{—}04934614.1, ZP_{—}07262554.1, YP_{—}786611.1, YP_{—}003439146.1, YP_{—}003592852.1, YP_{—}001747891.1, YP_{—}004386570.1, Q51956.1, YP_{—}004593695.1, YP_{—}560516.1, ZP_{—}06731844.1, YP_{—}001897101.1, ZP_{—}08388430.1, YP_{—}001166210.1, YP_{—}557015.1, ZP_{—}06843809.1, EGP42659.1, YP_{—}002005592.1, YP_{—}002871766.1, YP_{—}555845.1, ZP_{—}05921114.1, NP_{—}746745.1, NP_{—}637343.1, ZP_{—}02243308.1, YP_{—}001267798.1, ZP_{—}02354510.1, NP_{—}841567.1, ZP_{—}08177693.1, YP_{—}004703877.1, YP_{—}001166143.1, EGH73771.1, ZP_{—}06489206.1, ZP_{—}01892079.1, YP_{—}934562.1, ADY81955.1, EGB73439.1, NP_{—}520373.1, YP_{—}003905682.1, EGH61062.1, YP_{—}001894311.1, ZP_{—}02245330.1, YP_{—}918778.1, YP_{—}001120651.1, YP_{—}003612896.1, YP_{—}004125334.1, ZP_{—}07952596.1, YP_{—}001479268.1, ZP_{—}07043083.1, YP_{—}003644271.1, NP_{—}421210.1, ZP_{—}02882590.1, EGP25245.1, YP_{—}233920.1, EGD00226.1, YP_{—}004418315.1, ADY81914.1, ZP_{—}04944762.1, YP_{—}003603999.1, YP_{—}001060552.1, ZP_{—}03026966.1, YP_{—}441123.1, YP_{—}201177.1, ZP_{—}05117283.1, YP_{—}004232360.1, YP_{—}002802211.1, YP_{—}106085.1, YP_{—}258448.1, YP_{—}001989549.1, NP_{—}945866.1, ZP_{—}03573123.1, YP_{—}283604.1, YP_{—}004702122.1, ZP_{—}03398400.1, YP_{—}105310.1, YP_{—}001479310.1, CAC41637.1, ZP_{—}02372747.1, YP_{—}001578772.1, ZP_{—}08181762.1, ZP_{—}00439074.2, ADX92638.1, ZP_{—}03790444.1, YP_{—}110295.1, YP_{—}002439726.1, YP_{—}004361986.1, ZP_{—}04946665.1, YP_{—}003751825.1, YP_{—}001061488.1, ZP_{—}03545148.1, ZP_{—}01767462.1, ZP_{—}01769818.1, YP_{—}990241.1, YP_{—}002382777.1, YP_{—}002898389.1, YP_{—}003452421.1, EGH66881.1, CBW26817.1, YP_{—}004352451.1, EGC07165.1, YP_{—}003982691.1, ZP_{—}02906520.1, YP_{—}410799.1, YP_{—}001189077.1, YP_{—}004226900.1, ADP96997.1, YP_{—}237079.1, YP_{—}002946310.1, YP_{—}004029037.1, NP_{—}745423.1, ZP_{—}08139209.1, YP_{—}004294989.1, NP_{—}251630.1, EGH73592.1, ZP_{—}04934925.1, ZP_{—}03583227.1, ZP_{—}03584241.1, YP_{—}004684330.1, YP_{—}004501533.1, YP_{—}001186637.1, YP_{—}003980170.1, AEJ98540.1, YP_{—}004688333.1, YP_{—}003276725.1, EGH11975.1, YP_{—}276147.1, YP_{—}790233.1, ZP_{—}01736635.1, YP_{—}002440908.1, YP_{—}002230040.1, YP_{—}724980.1, YP_{—}004231717.1, ZP_{—}01226775.1, ZP_{—}03454556.1, ZP_{—}05586076.1, ZP_{—}02890239.1, YP_{—}001172996.1, ZP_{—}02377875.1, ZP_{—}07202399.1, YP_{—}774661.1, YP_{—}440314.2, YP_{—}443408.1, ZP_{—}06878044.1, ZP_{—}08274339.1, YP_{—}001618203.1, ZP_{—}08631485.1, ZP_{—}01545529.1, ZP_{—}03267843.1, ZP_{—}07797009.1, YP_{—}003376084.1, EGH21143.1, YP_{—}003753513.1, YP_{—}004282234.1, YP_{—}726356.1, ZP_{—}06014951.1, YP_{—}109637.1, ZP_{—}06461447.1, YP_{—}001795795.1, YP_{—}621981.1, ZP_{—}07794257.1, ZP_{—}05060451.1, YP_{—}002919830.1, YP_{—}001796645.1, NP_{—}794063.1, ZP_{—}01365347.1, YP_{—}003610065.1, YP_{—}001462757.1, YP_{—}001807185.1, ZP_{—}04928407.1, YP_{—}002229986.1, ZP_{—}02883901.1, YP_{—}370284.1, ZP_{—}05053491.1, AAC24332.1, ZP_{—}04929241.1, ZP_{—}00943679.1, YP_{—}001766064.1, YP_{—}001670661.1, YP_{—}003296167.1, YP_{—}003773673.1, NP_{—}250691.1, ZP_{—}05823066.1, YP_{—}004381309.1, YP_{—}004714773.1, YP_{—}746962.1, YP_{—}002513585.1, YP_{—}294674.1, YP_{—}004593822.1, YP_{—}622032.1, YP_{—}001897940.1, YP_{—}001335713.1, YP_{—}001856626.1, YP_{—}791238.1, YP_{—}004140309.1, YP_{—}001269802.1, ZP_{—}06879064.1, ZP_{—}01736318.1, ZP_{—}02886139.1, ZP_{—}04941413.1, YP_{—}001670851.1, YP_{—}371023.1, YP_{—}002980343.1, YP_{—}002795605.1, ZP_{—}06069679.1, ZP_{—}02463309.1, ZP_{—}05785212.1, YP_{—}001793049.1, YP_{—}003965283.1, YP_{—}001233153.1, YP_{—}299776.1, ZP_{—}06498740.1, AEJ99148.1, YP_{—}004685690.1, YP_{—}003746771.1, YP_{—}004381943.1, YP_{—}004378973.1, YP_{—}004314684.1, EGH79619.1, ZP_{—}04882546.1, YP_{—}347001.1, YP_{—}347471.1, YP_{—}001757758.1, YP_{—}002911324.1, NP_{—}518596.1, ZP_{—}00948908.1, YP_{—}442777.1, YP_{—}002874183.1, YP_{—}002230989.1, YP_{—}004360850.1, ABC36127.1, YP_{—}004696127.1, YP_{—}002799527.1, YP_{—}001631275.1, YP_{—}626125.1, ZP_{—}05090649.1, ZP_{—}07774002.1, ZP_{—}04940525.1, AEK60371.1, ADR60119.1, YP_{—}102981.1, YP_{—}003451423.1, NP_{—}743536.1, CAA45255.1,
in particular
YP_{—}790159.1, ZP_{—}06877967.1, NP_{—}251703.1, EGH66370.1, EGH10832.1, YP_{—}349606.1, YP_{—}236359.1, ZP_{—}07263340.1, EGH95844.1, NP_{—}793296.1, 1WDK_C, P28790.2, YP_{—}259060.1, ZP_{—}04587908.1, EGH45981.1, ZP_{—}07774144.1, EGH84449.1, YP_{—}275369.1, ZP_{—}07005686.1, YP_{—}001172247.1, YP_{—}004352962.1, ACP17922.1, YP_{—}002871196.1, ZP_{—}08142929.1, YP_{—}004701153.1, YP_{—}001667916.1, YP_{—}001268913.1, Q93Q11.1, Q9R9W0.1, NP_{—}744286.1, YP_{—}001187077.1, YP_{—}609234.1, YP_{—}004379417.1, YP_{—}004474975.1, ABP88737.1, EGH76239.1, EGH29725.1, ZP_{—}06499584.1, YP_{—}001749490.1, ABF82237.1, YP_{—}004713534.1, CAD76924.1, YP_{—}608369.1, AEA83130.1, YP_{—}001171793.1, YP_{—}788379.1, CAE45106.1, NP_{—}248919.1, YP_{—}001348923.1, ZP_{—}07797976.1, YP_{—}001747677.1, NP_{—}250427.1, AAR83740.1, YP_{—}001668851.1, ZP_{—}06495825.1, YP_{—}004379898.1, EGH60624.1, ZP_{—}04933402.1, YP_{—}001345710.1, Q8VPF1.1, YP_{—}004473788.1, EGH77345.1, EGH44350.1, YP_{—}001188713.1, YP_{—}004380648.1, YP_{—}001188079.1, ZP_{—}08142248.1, YP_{—}004703691.1, ZP_{—}04936650.1, YP_{—}606872.1, YP_{—}610092.1, ADR61907.1, ZP_{—}04934614.1, ZP_{—}07262554.1, YP_{—}001747891.1, Q51956.1, YP_{—}002871766.1, NP_{—}746745.1, YP_{—}001267798.1, YP_{—}004703877.1, EGH73771.1, EGH61062.1, YP_{—}233920.1, YP_{—}258448.1, YP_{—}004702122.1, ZP_{—}03398400.1, YP_{—}002439726.1, EGH66881.1, YP_{—}004352451.1, YP_{—}001189077.1, YP_{—}237079.1, NP_{—}745423.1, ZP_{—}08139209.1, NP_{—}251630.1, EGH73592.1, ZP_{—}04934925.1, YP_{—}001186637.1, EGH11975.1, YP_{—}276147.1, YP_{—}790233.1, YP_{—}002440908.1, YP_{—}001172996.1, ZP_{—}06878044.1, ZP_{—}07797009.1, EGH21143.1, ZP_{—}06461447.1, ZP_{—}07794257.1, NP_{—}794063.1, ZP_{—}01365347.1, ZP_{—}04928407.1, AAC24332.1, ZP_{—}04929241.1, YP_{—}001670661.1, NP_{—}250691.1, YP_{—}004381309.1, YP_{—}004714773.1, YP_{—}791238.1, YP_{—}001269802.1, ZP_{—}06879064.1, YP_{—}001670851.1, ZP_{—}06498740.1, YP_{—}004381943.1, YP_{—}004378973.1, EGH79619.1, YP_{—}347001.1, YP_{—}347471.1, YP_{—}002874183.1, ZP_{—}07774002.1, ADR60119.1, NP_{—}743536.1, YP_{—}001269653.1, ZP_{—}06482365.1, ADI95330.1, ZP_{—}07003619.1, BAB96553.1, ZP_{—}07777009.1, ABA10831.1, YP_{—}273131.1, YP_{—}259428.1, EFW86233.1, EGH85840.1, ZP_{—}07774597.1, EGH54613.1, YP_{—}004353129.1, YP_{—}002871014.1, YP_{—}001171232.1, EGH67454.1, EFW82139.1, ZP_{—}04590526.1, EGH58132.1, EGH06629.1, EGH99157.1, ZP_{—}05638078.1, NP_{—}790796.1, AEE59172.1, YP_{—}026272.1, YP_{—}002389323.1, EGB30581.1, YP_{—}001460637.1, YP_{—}001727089.1, CAB40810.1, ZP_{—}03049054.1, ZP_{—}08356522.1, ZP_{—}06664573.1, AAA67642.1, YP_{—}003236969.1, ZP_{—}06659935.1, NP_{—}290475.1, ZP_{—}03027945.1, EFZ59092.1, YP_{—}002295406.1, CBG37050.1, EGP22872.1, EGE62411.1, YP_{—}002400349.1, EGB59498.1, ZP_{—}08361144.1, YP_{—}001465329.1, YP_{—}002409079.1, ZP_{—}06655947.1, YP_{—}002414995.1, EGB69559.1, YP_{—}859446.1, ZP_{—}08386168.1, YP_{—}543378.1, ZP_{—}08366394.1, YP_{—}001746177.1, ZP_{—}07154548.1, ZP_{—}03070708.1, NP_{—}756651.1, YP_{—}671919.1, YP_{—}002331615.1, ZP_{—}07449249.1, ZP_{—}06938722.1, ZP_{—}03086141.1, ZP_{—}07189177.1, ZP_{—}07136312.1, EFZ47010.1, ZP_{—}07136311.1, EFZ47009.1, ZP_{—}03026937.1, EGB73439.1, EGP25245.1, ZP_{—}03026966.1, YP_{—}001462757.1, CAP76727.1, YP_{—}670163.1,
and particularly preferably
YP_{—}026272.1, AAA67642.1, ZP_{—}07593202.1, YP_{—}004701153.1, YP_{—}001667916.1, YP_{—}001268913.1, Q9R9W0.1, NP_{—}744286.1, ABP88737.1, YP_{—}001749490.1, YP_{—}001747677.1, YP_{—}001668851.1, YP_{—}004703691.1, ADR61907.1, YP_{—}001747891.1, Q51956.1, NP_{—}746745.1, YP_{—}001267798.1, YP_{—}004703877.1, YP_{—}004702122.1, NP_{—}745423.1, AAC24332.1, YP_{—}001670661.1, YP_{—}001269802.1, YP_{—}001670851.1, ADR60119.1, NP_{—}743536.1, YP_{—}001269653.1, ADI95330.1, BAB96553.1, YP_{—}001172247.1, YP_{—}004713534.1, AEA83130.1, YP_{—}001171793.1, YP_{—}001172996.1, YP_{—}004714773.1, YP_{—}001171232.1, YP_{—}349606.1, YP_{—}259060.1, ZP_{—}07774144.1, YP_{—}002871196.1, ABF82237.1, YP_{—}002871766.1, YP_{—}258448.1, YP_{—}347001.1, YP_{—}347471.1, YP_{—}002874183.1, ZP_{—}07774002.1, ZP_{—}07777009.1, YP_{—}259428.1, ZP_{—}07774597.1, YP_{—}002871014.1,
and
proteins with a polypeptide sequence wherein up to 60%, preferably up to 25%, particularly preferably up to 15%, in particular up to 10, 9, 8, 7, 6, 5, 4, 3, 2 or 1% of the amino acid residues compared to the aforementioned reference sequences are modified by deletion, insertion, substitution or a combination thereof and which still possess at least 50%, preferably 65%, particularly preferably 80%, in particular more than 90% of the activity of the protein with the corresponding, aforementioned reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection and in connection with the determination of the activity of the enzyme E_{f} in general is understood to mean in particular the conversion of 3-oxo-dodecanoyl-CoA thioesters and CoA to decanoyl-CoA thioesters and acetyl-CoA.

### Sixth Genetic Modification for Intensification of the Acyl-ACP Thioester Synthesis

According to the invention, the microorganisms have a sixth genetic modification, so that compared to their wild type they are capable of forming more acyl-ACP thioester from at least one simple carbon source. An overview of correspondingly desirable genetic modifications is to be found in FIG. 1 of WO2008119082, paragraph 1 (Fatty Acid Production Increase/Product Production Increase).

This has the technical effect that formation of carboxylic acids and carboxylate ester intensified by the first genetic modification, but also of ω-functionalized carboxylic acids and carboxylate esters formed in greater quantity through the second, third, fourth, fifth or seventh genetic modification, is still further intensified.

Thus configured organisms preferred according to the invention are also outstandingly suitable for the production of compounds which are selected from

α,ω-alkanediols, α,ω-alkanedialdehydes, α-oxo-ω-hydroxyalkanes and α,ω-alkanediamines, since compounds of these classes are produced in significant quantities together with ω-functionalized alkanoic acids and ω-functionalized alkanoate esters
Seventh Genetic Modification for the Production of ω-Functionalized Carboxylic Acids and Co-Functionalized Carboxylate Esters with a Terminal Double Bond

The microorganisms according to the invention can additionally be configured such that they are advantageously suitable for the production of ω-functionalized carboxylic acids and ω-functionalized carboxylate esters with a terminal double bond. For this, preferred microorganisms contain a seventh genetic modification which comprises, compared to the enzymatic activity of the wild type of the microorganism, increased activity of an enzyme Eₓᵢ which catalyses the conversion of ω-carboxycarboxylic acids or ω-carboxycarboxylate esters to carboxylic acids or carboxylate esters with a terminal double bond, selected from the group Eₓᵢ) Cytochrome P450 fatty acid decarboxylase, which catalyses the conversion of an alkanoic acid with n carbon atoms to a corresponding terminal olefin with n−1 carbon atoms, in particular of dodecanoic acid to undec-10-enoic acid.

Microorganisms preferred according to the invention are those which are obtained when the microorganisms listed below having a seventh genetic modification in the sense of the invention are used as the starting point, in that they are provided with a first and second genetic modification and optionally at least one further genetic modification in the sense of the invention.

WO2009085278 A1 describes, particularly in paragraphs [0033] to [0048], [0056] to [0063] and [0188] to [0202], FIG. 10, table 8, practical examples 5 to 18 and claims 28 to 51 and 188 to 195, microorganisms preferred according to the invention which have a seventh genetic modification, so that compared to their wild type they are capable of forming more fatty acids and fatty acid derivatives, in particular olefins, from at least one simple carbon source. The document also describes Eₓᵢ preferred according to the invention enzymes and the sequences thereof, particularly in paragraphs [0021] to [0032], [0051] to [0055], [0081] to [0084] and [0160] to [0183], table 8, practical examples 5 to 18, claims 1 to 25 and FIGS. 3, 7 and 9.

### Specific Embodiments of Preferable Microorganisms and Enzymes

According to the invention, microorganisms are particularly preferably selected from those which have

a first and a second genetic modification in the sense of the invention,
a first, a second and a fifth genetic modification in the sense of the invention,
a first, a second and a third genetic modification in the sense of the invention,
a first, a second, a third and a fifth genetic modification in the sense of the invention,
a first, a second and a fourth genetic modification in the sense of the invention,
a first, a second, a fourth and a fifth genetic modification in the sense of the invention,
a first, a second, a third and a fourth genetic modification in the sense of the invention,
a first, a second, a third, a fourth and a fifth genetic modification in the sense of the invention,
a first, a second and a seventh genetic modification in the sense of the invention,
a first, a second, a fifth and a seventh genetic modification in the sense of the invention,
a first, a second, a third and a seventh genetic modification in the sense of the invention or
a first, a second, a third, a fifth and a seventh genetic modification in the sense of the invention
a first, a second and a sixth genetic modification in the sense of the invention,
a first, a second, a fifth and a sixth genetic modification in the sense of the invention,
a first, a second, a third and a sixth genetic modification in the sense of the invention,
a first, a second, a third, a fifth and a sixth genetic modification in the sense of the invention,
a first, a second, a fourth and a sixth genetic modification in the sense of the invention,
a first, a second, a fourth, a fifth and a sixth genetic modification in the sense of the invention,
a first, a second, a third, a fourth and a sixth genetic modification in the sense of the invention,
a first, a second, a third, a fourth, a fifth and a sixth genetic modification in the sense of the invention,
a first, a second, a sixth and a seventh genetic modification in the sense of the invention,
a first, a second, a fifth, a sixth and a seventh genetic modification in the sense of the invention,
a first, a second, a third, a sixth and a seventh genetic modification in the sense of the invention or
a first, a second, a third, a fifth, sixth and a seventh genetic modification in the sense of the invention.

According to the invention, microorganisms are particularly preferable which have a first genetic modification, so that compared to their wild type they are capable of forming more carboxylic acids and carboxylate ester from at least one simple carbon source, wherein the first genetic modification displays, compared to the enzymatic activity of the wild type of the microorganism, increased activity of at least one of the enzymes Eᵢ or of one of the enzymes with a polypeptide sequence wherein up to 60%, preferably up to 25%, particularly preferably up to 15%, in particular up to 10, 9, 8, 7, 6, 5, 4, 3, 2 or 1% amino acid residues compared to the sequences stated in the following table by reference are modified by deletion, insertion, substitution or a combination thereof and which still possess at least 50%, preferably 65%, particularly preferably 80%, in particular more than 90% of the activity of the protein with the respective reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein, wherein the activity in this connection in general is understood to mean in particular the hydrolysis of an ACP thioester with the carbon chain length assigned in the following table to the individual enzymes Eᵢ, and the carboxylic acid and carboxylate ester have a carbon chain length of the carboxylic acid part, as shown in the following table:

| Enzyme Eᵢ selected from | Carbon chain length |
|---|---|
| AAC49269.1, CAB60830.1, AAC49179.1, | C8 |
| AAC49784.1, ABB71579.1, CAC19934.1 | |
| and SEQ ID Nos. 26, 29, 33, 38, 40, 97 and | |
| 99 of WO2011008565 | |
| AAC49269.1, CAB60830.1, AAC49179.1, | C10 |
| AAC49784.1, ABB71579.1, CAC19934.1 | |
| and SEQ ID Nos. 73, 75, 87 and 89 of | |
| WO2011008565. | |
| Q41635.1, Q39473.1, AAC49180.1, | C12 |
| CAC19934.1, AAC72881.1, AAC49783.1, | |
| AAC49784.1 and SEQ ID Nos. 49 and 51 of | |
| WO2011008565. | |
| Q41635.1, Q39473.1, AAC49180.1, | C14 |
| CAC19934.1, AAC72881.1 AAC49783.1, | |
| AAC49784.1 and SEQ ID Nos. 49, 51, 53, | |
| 55, 61, 63, 67, 69, 77, 79, 83 and 85 of | |
| WO2011008565. | |

The aforementioned deletions of amino acid residues compared to the sequences stated in the above table by reference relate in particular to deletions at the N- and/or C-terminus, in particular at the N-Terminus. Particularly preferably, the aforementioned N-terminus is that of a plant plastid targeting sequence. Such plant plastid targeting sequences can for example be predicted by means of the algorithms utilized by the predictive tool TargetP 1.1 (www.cbs.dtu.dk/servicesiTargetP/) and described in the following publications, preferably without use of cutoffs:

Predicting Subcellular Localization of Proteins Based on their N-Terminal Amino Acid Sequence.

Olof Emanuelsson, Henrik Nielsen, Soren Brunak and Gunnar von Heijne.

J. Mol. Biol., 300: 1005-1016, 2000 and Identification of prokaryotic and eukaryotic signal peptides and prediction of their cleavage sites. Henrik Nielsen, Jacob Engelbrecht, Soren Brunak and Gunnar von Heijne. Protein Engineering, 10:1-6, 1997.

Microorganisms quite especially preferred according to the invention (abbreviated as MO) are outstandingly suitable for the production of ω-aminocarboxylic acids and have increased or decreased enzyme activities (abbreviated as E) described in the following table, where these can in addition advantageously be combined with an increased enzyme activity compared to the wild type of the microorganism, which is described for the 3-ketoacyl-ACP (Acyl Carrrier Protein) synthase III (EC 2.3.1.41), in particular that from plants, preferably that from plants the seeds whereof contain fatty acids with alkyl residues shorter than 14 C atoms, and particularly preferably that from plants of the genera Cuphea, Elaeis, Cocos, Umbellularia and Cinnamomum and gene products selected from AccA, AccB, AccC, AccD, AceE, AceF, Lpd, AcpP, FabA, FabB, FabD, FabF, FabG, FabH, Fabl, FabZ, PanD, PanK, UdhA, PntA or PntB. Any combinations of at least two of these enzyme activities can advantageously be increased.

In addition, moreover, it can be advantageous if the microorganism is provided with a lower enzyme activity compared to the wild type of the microorganism, which is described for the gene products selected from TdcE, PflA, PflB, PflC, PflD, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, singly or in any combination.

| | | |
|---|---|---|
| MO | E increased | E decreased |

| | | | | |
|---|---|---|---|---|
| 1 | E₁ₐE₂ | | | |
| 2 | E_{1b}E₂ | | | |
| 3 | E₁ₐE₂E₃ | | | |
| 4 | E_{1b}E₂E₃ | | | |
| 5 | E₁ₐE₂Eᵢ | | | |
| 6 | E_{1b}E₂Eᵢ | | | |
| 7 | E₁ₐE₂Eᵢᵢ | | | |
| 8 | E_{1b}E₂Eᵢᵢ | | | |
| 9 | E₁ₐE₂EᵢᵢE_{iib} | | | |
| 10 | E_{1b}E₂EᵢᵢE_{iib} | | | |
| 11 | E₁ₐE₂E₃Eᵢ | | | |
| 12 | E_{1b}E₂E₃Eᵢ | | | |
| 13 | E₁ₐE₂E₃Eᵢᵢ | | | |
| 14 | E_{1b}E₂E₃Eᵢᵢ | | | |
| 15 | E₁ₐE₂E₃EᵢᵢE_{iib} | | | |
| 16 | E_{1b}E₂E₃EᵢᵢE_{iib} | | | |
| 17 | E₁ₐE₂E₄ | | | |
| 18 | E_{1b}E₂E₄ | | | |
| 19 | E₁ₐE₂E₃E₄ | | | |
| 20 | E_{1b}E₂E₃E₄ | | | |
| 21 | E₁ₐE₂EᵢE₄ | | | |
| 22 | E_{1b}E₂EᵢE₄ | | | |
| 23 | E₁ₐE₂EᵢᵢE₄ | | | |
| 24 | E_{1b}E₂EᵢᵢE₄ | | | |
| 25 | E₁ₐE₂EᵢᵢE_{iib}E₄ | | | |
| 26 | E_{1b}E₂EᵢᵢE_{iib}E₄ | | | |
| 27 | E₁ₐE₂E₃EᵢE₄ | | | |
| 28 | E_{1b}E₂E₃EᵢE₄ | | | |
| 29 | E₁ₐE₂E₃EᵢᵢE₄ | | | |
| 30 | E_{1b}E₂E₃EᵢᵢE₄ | | | |
| 31 | E₁ₐE₂E₃EᵢᵢE_{iib}E₄ | | | |
| 32 | E_{1b}E₂E₃EᵢᵢE_{iib}E₄ | | | |
| 33 | E₁ₐE₂Eᵢᵢᵢ | | | |
| 34 | E_{1b}E₂Eᵢᵢᵢ | | | |
| 35 | E₁ₐE₂E₃Eᵢᵢᵢ | | | |
| 36 | E_{1b}E₂E₃Eᵢᵢᵢ | | | |
| 37 | E₁ₐE₂EᵢᵢᵢE₄ | | | |
| 38 | E_{1b}E₂EᵢᵢᵢE₄ | | | |
| 39 | E₁ₐE₂E₃EᵢᵢᵢE₄ | | | |
| 40 | E_{1b}E₂E₃EᵢᵢᵢE₄ | | | |
| 41 | E₁ₐE₂Eᵢᵥ | | | |
| 42 | E_{1b}E₂Eᵢᵥ | | | |
| 43 | E₁ₐE₂E₃Eᵢᵥ | | | |
| 44 | E_{1b}E₂E₃Eᵢᵥ | | | |
| 45 | E₁ₐE₂EᵢᵥE₄ | | | |
| 46 | E_{1b}E₂EᵢᵥE₄ | | | |
| 47 | E₁ₐE₂E₃EᵢᵥE₄ | | | |
| 48 | E_{1b}E₂E₃EᵢᵥE₄ | | | |
| 49 | E₁ₐE₂ | | E₄* | |
| 50 | E_{1b}E₂ | | E₄* | |
| 51 | E₁ₐE₂E₃ | | E₄* | |
| 52 | E_{1b}E₂E₃ | | E₄* | |
| 53 | E₁ₐE₂Eᵢ | | E₄* | |
| 54 | E_{1b}E₂Eᵢ | | E₄* | |
| 55 | E₁ₐE₂Eᵢᵢ | | E₄* | |
| 56 | E_{1b}E₂Eᵢᵢ | | E₄* | |
| 57 | E₁ₐE₂EᵢᵢE_{iib} | | E₄* | |
| 58 | E_{1b}E₂EᵢᵢE_{iib} | | E₄* | |
| 59 | E₁ₐE₂E₃Eᵢ | | E₄* | |
| 60 | E_{1b}E₂E₃Eᵢ | | E₄* | |
| 61 | E₁ₐE₂E₃Eᵢᵢ | | E₄* | |
| 62 | E_{1b}E₂E₃Eᵢᵢ | | E₄* | |
| 63 | E₁ₐE₂E₃EᵢᵢE_{iib} | | E₄* | |
| 64 | E_{1b}E₂E₃EᵢᵢE_{iib} | | E₄* | |
| 65 | E₁ₐE₂E₄ | | E₄* | |
| 66 | E_{1b}E₂E₄ | | E₄* | |
| 67 | E₁ₐE₂E₃E₄ | | E₄* | |
| 68 | E_{1b}E₂E₃E₄ | | E₄* | |
| 69 | E₁ₐE₂EᵢE₄ | | E₄* | |
| 70 | E_{1b}E₂EᵢE₄ | | E₄* | |
| 71 | E₁ₐE₂EᵢᵢE₄ | | E₄* | |
| 72 | E_{1b}E₂EᵢᵢE₄ | | E₄* | |
| 73 | E₁ₐE₂EᵢᵢE_{iib}E₄ | | E₄* | |
| 74 | E_{1b}E₂EᵢᵢE_{iib}E₄ | | E₄* | |
| 75 | E₁ₐE₂E₃EᵢE₄ | | E₄* | |
| 76 | E_{1b}E₂E₃EᵢE₄ | | E₄* | |
| 77 | E₁ₐE₂E₃EᵢᵢE₄ | | E₄* | |
| 78 | E_{1b}E₂E₃EᵢᵢE₄ | | E₄* | |
| 79 | E₁ₐE₂E₃EᵢᵢE_{iib}E₄ | | E₄* | |
| 80 | E_{1b}E₂E₃EᵢᵢE_{iib}E₄ | | E₄* | |
| 81 | E₁ₐE₂Eᵢᵢᵢ | | E₄* | |
| 82 | E_{1b}E₂Eᵢᵢᵢ | | E₄* | |
| 83 | E₁ₐE₂E₃Eᵢᵢᵢ | | E₄* | |
| 84 | E_{1b}E₂E₃Eᵢᵢᵢ | | E₄* | |
| 85 | E₁ₐE₂EᵢᵢᵢE₄ | | E₄* | |
| 86 | E_{1b}E₂EᵢᵢᵢE₄ | | E₄* | |
| 87 | E₁ₐE₂E₃EᵢᵢᵢE₄ | | E₄* | |
| 88 | E_{1b}E₂E₃EᵢᵢᵢE₄ | | E₄* | |
| 89 | E₁ₐE₂Eᵢᵥ | | E₄* | |
| 90 | E_{1b}E₂Eᵢᵥ | | E₄* | |
| 91 | E₁ₐE₂E₃Eᵢᵥ | | E₄* | |
| 92 | E_{1b}E₂E₃Eᵢᵥ | | E₄* | |
| 93 | E₁ₐE₂EᵢᵥE₄ | | E₄* | |
| 94 | E_{1b}E₂EᵢᵥE₄ | | E₄* | |
| 95 | E₁ₐE₂E₃EᵢᵥE₄ | | E₄* | |
| 96 | E_{1b}E₂E₃EᵢᵥE₄ | | E₄* | |
| 97 | E₁ₐE₂ | | E₅ | |
| 98 | E_{1b}E₂ | | E₅ | |
| 99 | E₁ₐE₂E₃ | | E₅ | |
| 100 | E_{1b}E₂E₃ | | E₅ | |
| 101 | E₁ₐE₂Eᵢ | | E₅ | |
| 102 | E_{1b}E₂Eᵢ | | E₅ | |
| 103 | E₁ₐE₂Eᵢᵢ | | E₅ | |
| 104 | E_{1b}E₂Eᵢᵢ | | E₅ | |
| 105 | E₁ₐE₂EᵢᵢE_{iib} | | E₅ | |
| 106 | E_{1b}E₂EᵢᵢE_{iib} | | E₅ | |
| 107 | E₁ₐE₂E₃Eᵢ | | E₅ | |
| 108 | E_{1b}E₂E₃Eᵢ | | E₅ | |
| 109 | E₁ₐE₂E₃Eᵢᵢ | | E₅ | |
| 110 | E_{1b}E₂E₃Eᵢᵢ | | E₅ | |
| 111 | E₁ₐE₂E₃EᵢᵢE_{iib} | | E₅ | |
| 112 | E_{1b}E₂E₃EᵢᵢE_{iib} | | E₅ | |
| 113 | E₁ₐE₂E₄ | | E₅ | |
| 114 | E_{1b}E₂E₄ | | E₅ | |
| 115 | E₁ₐE₂E₃E₄ | | E₅ | |
| 116 | E_{1b}E₂E₃E₄ | | E₅ | |
| 117 | E₁ₐE₂EᵢE₄ | | E₅ | |
| 118 | E_{1b}E₂EᵢE₄ | | E₅ | |
| 119 | E₁ₐE₂EᵢᵢE₄ | | E₅ | |
| 120 | E_{1b}E₂EᵢᵢE₄ | | E₅ | |
| 121 | E₁ₐE₂EᵢᵢE_{iib}E₄ | | E₅ | |
| 122 | E_{1b}E₂EᵢᵢE_{iib}E₄ | | E₅ | |
| 123 | E₁ₐE₂E₃EᵢE₄ | | E₅ | |
| 124 | E_{1b}E₂E₃EᵢE₄ | | E₅ | |
| 125 | E₁ₐE₂E₃EᵢᵢE₄ | | E₅ | |
| 126 | E_{1b}E₂E₃EᵢᵢE₄ | | E₅ | |
| 127 | E₁ₐE₂E₃EᵢᵢE_{iib}E₄ | | E₅ | |
| 128 | E_{1b}E₂E₃EᵢᵢE_{iib}E₄ | | E₅ | |
| 129 | E₁ₐE₂Eᵢᵢᵢ | | E₅ | |
| 130 | E_{1b}E₂Eᵢᵢᵢ | | E₅ | |
| 131 | E₁ₐE₂E₃Eᵢᵢᵢ | | E₅ | |
| 132 | E_{1b}E₂E₃Eᵢᵢᵢ | | E₅ | |
| 133 | E₁ₐE₂EᵢᵢᵢE₄ | | E₅ | |
| 134 | E_{1b}E₂EᵢᵢᵢE₄ | | E₅ | |
| 135 | E₁ₐE₂E₃EᵢᵢᵢE₄ | | E₅ | |
| 136 | E_{1b}E₂E₃EᵢᵢᵢE₄ | | E₅ | |
| 137 | E₁ₐE₂Eᵢᵥ | | E₅ | |
| 138 | E_{1b}E₂Eᵢᵥ | | E₅ | |
| 139 | E₁ₐE₂E₃Eᵢᵥ | | E₅ | |
| 140 | E_{1b}E₂E₃Eᵢᵥ | | E₅ | |
| 141 | E₁ₐE₂EᵢᵥE₄ | | E₅ | |
| 142 | E_{1b}E₂EᵢᵥE₄ | | E₅ | |
| 143 | E₁ₐE₂E₃EᵢᵥE₄ | | E₅ | |
| 144 | E_{1b}E₂E₃EᵢᵥE₄ | | E₅ | |
| 145 | E₁ₐE₂ | | E₄*E₅ | |
| 146 | E_{1b}E₂ | | E₄*E₅ | |
| 147 | E₁ₐE₂E₃ | | E₄*E₅ | |
| 148 | E_{1b}E₂E₃ | | E₄*E₅ | |
| 149 | E₁ₐE₂Eᵢ | | E₄*E₅ | |
| 150 | E_{1b}E₂Eᵢ | | E₄*E₅ | |
| 151 | E₁ₐE₂Eᵢᵢ | | E₄*E₅ | |
| 152 | E_{1b}E₂Eᵢᵢ | | E₄*E₅ | |
| 153 | E₁ₐE₂EᵢᵢE_{iib} | | E₄*E₅ | |
| 154 | E_{1b}E₂EᵢᵢE_{iib} | | E₄*E₅ | |
| 155 | E₁ₐE₂E₃Eᵢ | | E₄*E₅ | |
| 156 | E_{1b}E₂E₃Eᵢ | | E₄*E₅ | |
| 157 | E₁ₐE₂E₃Eᵢᵢ | | E₄*E₅ | |
| 158 | E_{1b}E₂E₃Eᵢᵢ | | E₄*E₅ | |
| 159 | E₁ₐE₂E₃EᵢᵢE_{iib} | | E₄*E₅ | |
| 160 | E_{1b}E₂E₃EᵢᵢE_{iib} | | E₄*E₅ | |
| 161 | E₁ₐE₂E₄ | | E₄*E₅ | |
| 162 | E_{1b}E₂E₄ | | E₄*E₅ | |
| 163 | E₁ₐE₂E₃E₄ | | E₄*E₅ | |
| 164 | E_{1b}E₂E₃E₄ | | E₄*E₅ | |
| 165 | E₁ₐE₂EᵢE₄ | | E₄*E₅ | |
| 166 | E_{1b}E₂EᵢE₄ | | E₄*E₅ | |
| 167 | E₁ₐE₂EᵢᵢE₄ | | E₄*E₅ | |
| 168 | E_{1b}E₂EᵢᵢE₄ | | E₄*E₅ | |
| 169 | E₁ₐE₂EᵢᵢE_{iib}E₄ | | E₄*E₅ | |
| 170 | E_{1b}E₂EᵢᵢE_{iib}E₄ | | E₄*E₅ | |
| 171 | E₁ₐE₂E₃EᵢE₄ | | E₄*E₅ | |
| 172 | E_{1b}E₂E₃EᵢE₄ | | E₄*E₅ | |
| 173 | E₁ₐE₂E₃EᵢᵢE₄ | | E₄*E₅ | |
| 174 | E_{1b}E₂E₃EᵢᵢE₄ | | E₄*E₅ | |
| 175 | E₁ₐE₂E₃EᵢᵢE_{iib}E₄ | | E₄*E₅ | |
| 176 | E_{1b}E₂E₃EᵢᵢE_{iib}E₄ | | E₄*E₅ | |
| 177 | E₁ₐE₂Eᵢᵢᵢ | | E₄*E₅ | |
| 178 | E_{1b}E₂Eᵢᵢᵢ | | E₄*E₅ | |
| 179 | E₁ₐE₂E₃Eᵢᵢᵢ | | E₄*E₅ | |
| 180 | E_{1b}E₂E₃Eᵢᵢᵢ | | E₄*E₅ | |
| 181 | E₁ₐE₂EᵢᵢᵢE₄ | | E₄*E₅ | |
| 182 | E_{1b}E₂EᵢᵢᵢE₄ | | E₄*E₅ | |
| 183 | E₁ₐE₂E₃EᵢᵢᵢE₄ | | E₄*E₅ | |
| 184 | E_{1b}E₂E₃EᵢᵢᵢE₄ | | E₄*E₅ | |
| 185 | E₁ₐE₂Eᵢᵥ | | E₄*E₅ | |
| 186 | E_{1b}E₂Eᵢᵥ | | E₄*E₅ | |
| 187 | E₁ₐE₂E₃Eᵢᵥ | | E₄*E₅ | |
| 188 | E_{1b}E₂E₃Eᵢᵥ | | E₄*E₅ | |
| 189 | E₁ₐE₂EᵢᵥE₄ | | E₄*E₅ | |
| 190 | E_{1b}E₂EᵢᵥE₄ | | E₄*E₅ | |
| 191 | E₁ₐE₂E₃EᵢᵥE₄ | | E₄*E₅ | |
| 192 | E_{1b}E₂E₃EᵢᵥE₄ | | E₄*E₅ | |
| 193 | E₁ₐE₂ | AlkL | | |
| 194 | E_{1b}E₂ | AlkL | | |
| 195 | E₁ₐE₂E₃ | AlkL | | |
| 196 | E_{1b}E₂E₃ | AlkL | | |
| 197 | E₁ₐE₂Eᵢ | AlkL | | |
| 198 | E_{1b}E₂Eᵢ | AlkL | | |
| 199 | E₁ₐE₂Eᵢᵢ | AlkL | | |
| 200 | E_{1b}E₂Eᵢᵢ | AlkL | | |
| 201 | E₁ₐE₂EᵢᵢE_{iib} | AlkL | | |
| 202 | E_{1b}E₂EᵢᵢE_{iib} | AlkL | | |
| 203 | E₁ₐE₂E₃Eᵢ | AlkL | | |
| 204 | E_{1b}E₂E₃Eᵢ | AlkL | | |
| 205 | E₁ₐE₂E₃Eᵢᵢ | AlkL | | |
| 206 | E_{1b}E₂E₃Eᵢᵢ | AlkL | | |
| 207 | E₁ₐE₂E₃EᵢᵢE_{iib} | AlkL | | |
| 208 | E_{1b}E₂E₃EᵢᵢE_{iib} | AlkL | | |
| 209 | E₁ₐE₂E₄ | AlkL | | |
| 210 | E_{1b}E₂E₄ | AlkL | | |
| 211 | E₁ₐE₂E₃E₄ | AlkL | | |
| 212 | E_{1b}E₂E₃E₄ | AlkL | | |
| 213 | E₁ₐE₂EᵢE₄ | AlkL | | |
| 214 | E_{1b}E₂EᵢE₄ | AlkL | | |
| 215 | E₁ₐE₂EᵢᵢE₄ | AlkL | | |
| 216 | E_{1b}E₂EᵢᵢE₄ | AlkL | | |
| 217 | E₁ₐE₂EᵢᵢE_{iib}E₄ | AlkL | | |
| 218 | E_{1b}E₂EᵢᵢE_{iib}E₄ | AlkL | | |
| 219 | E₁ₐE₂E₃EᵢE₄ | AlkL | | |
| 220 | E_{1b}E₂E₃EᵢE₄ | AlkL | | |
| 221 | E₁ₐE₂E₃EᵢᵢE₄ | AlkL | | |
| 222 | E_{1b}E₂E₃EᵢᵢE₄ | AlkL | | |
| 223 | E₁ₐE₂E₃EᵢᵢE_{iib}E₄ | AlkL | | |
| 224 | E_{1b}E₂E₃EᵢᵢE_{iib}E₄ | AlkL | | |
| 225 | E₁ₐE₂Eᵢᵢᵢ | AlkL | | |
| 226 | E_{1b}E₂Eᵢᵢᵢ | AlkL | | |
| 227 | E₁ₐE₂E₃Eᵢᵢᵢ | AlkL | | |
| 228 | E_{1b}E₂E₃Eᵢᵢᵢ | AlkL | | |
| 229 | E₁ₐE₂EᵢᵢᵢE₄ | AlkL | | |
| 230 | E_{1b}E₂EᵢᵢᵢE₄ | AlkL | | |
| 231 | E₁ₐE₂E₃EᵢᵢᵢE₄ | AlkL | | |
| 232 | E_{1b}E₂E₃EᵢᵢᵢE₄ | AlkL | | |
| 233 | E₁ₐE₂Eᵢᵥ | AlkL | | |
| 234 | E_{1b}E₂Eᵢᵥ | AlkL | | |
| 235 | E₁ₐE₂E₃Eᵢᵥ | AlkL | | |
| 236 | E_{1b}E₂E₃Eᵢᵥ | AlkL | | |
| 237 | E₁ₐE₂EᵢᵥE₄ | AlkL | | |
| 238 | E_{1b}E₂EᵢᵥE₄ | AlkL | | |
| 239 | E₁ₐE₂E₃EᵢᵥE₄ | AlkL | | |
| 240 | E_{1b}E₂E₃EᵢᵥE₄ | AlkL | | |
| 241 | E₁ₐE₂ | AlkL | E₄* | |
| 242 | E_{1b}E₂ | AlkL | E₄* | |
| 243 | E₁ₐE₂E₃ | AlkL | E₄* | |
| 244 | E_{1b}E₂E₃ | AlkL | E₄* | |
| 245 | E₁ₐE₂Eᵢ | AlkL | E₄* | |
| 246 | E_{1b}E₂Eᵢ | AlkL | E₄* | |
| 247 | E₁ₐE₂Eᵢᵢ | AlkL | E₄* | |
| 248 | E_{1b}E₂Eᵢᵢ | AlkL | E₄* | |
| 249 | E₁ₐE₂EᵢᵢE_{iib} | AlkL | E₄* | |
| 250 | E_{1b}E₂EᵢᵢE_{iib} | AlkL | E₄* | |
| 251 | E₁ₐE₂E₃Eᵢ | AlkL | E₄* | |
| 252 | E_{1b}E₂E₃Eᵢ | AlkL | E₄* | |
| 253 | E₁ₐE₂E₃Eᵢᵢ | AlkL | E₄* | |
| 254 | E_{1b}E₂E₃Eᵢᵢ | AlkL | E₄* | |
| 255 | E₁ₐE₂E₃EᵢᵢE_{iib} | AlkL | E₄* | |
| 256 | E_{1b}E₂E₃EᵢᵢE_{iib} | AlkL | E₄* | |
| 257 | E₁ₐE₂E₄ | AlkL | E₄* | |
| 258 | E_{1b}E₂E₄ | AlkL | E₄* | |
| 259 | E₁ₐE₂E₃E₄ | AlkL | E₄* | |
| 260 | E_{1b}E₂E₃E₄ | AlkL | E₄* | |
| 261 | E₁ₐE₂EᵢE₄ | AlkL | E₄* | |
| 262 | E_{1b}E₂EᵢE₄ | AlkL | E₄* | |
| 263 | E₁ₐE₂EᵢᵢE₄ | AlkL | E₄* | |
| 264 | E_{1b}E₂EᵢᵢE₄ | AlkL | E₄* | |
| 265 | E₁ₐE₂EᵢᵢE_{iib}E₄ | AlkL | E₄* | |
| 266 | E_{1b}E₂EᵢᵢE_{iib}E₄ | AlkL | E₄* | |
| 267 | E₁ₐE₂E₃EᵢE₄ | AlkL | E₄* | |
| 268 | E_{1b}E₂E₃EᵢE₄ | AlkL | E₄* | |
| 269 | E₁ₐE₂E₃EᵢᵢE₄ | AlkL | E₄* | |
| 270 | E_{1b}E₂E₃EᵢᵢE₄ | AlkL | E₄* | |
| 271 | E₁ₐE₂E₃EᵢᵢE_{iib}E₄ | AlkL | E₄* | |
| 272 | E_{1b}E₂E₃EᵢᵢE_{iib}E₄ | AlkL | E₄* | |
| 273 | E₁ₐE₂Eᵢᵢᵢ | AlkL | E₄* | |
| 274 | E_{1b}E₂Eᵢᵢᵢ | AlkL | E₄* | |
| 275 | E₁ₐE₂E₃Eᵢᵢᵢ | AlkL | E₄* | |
| 276 | E_{1b}E₂E₃Eᵢᵢᵢ | AlkL | E₄* | |
| 277 | E₁ₐE₂EᵢᵢᵢE₄ | AlkL | E₄* | |
| 278 | E_{1b}E₂EᵢᵢᵢE₄ | AlkL | E₄* | |
| 279 | E₁ₐE₂E₃EᵢᵢᵢE₄ | AlkL | E₄* | |
| 280 | E_{1b}E₂E₃EᵢᵢᵢE₄ | AlkL | E₄* | |
| 281 | E₁ₐE₂Eᵢᵥ | AlkL | E₄* | |
| 282 | E_{1b}E₂Eᵢᵥ | AlkL | E₄* | |
| 283 | E₁ₐE₂E₃Eᵢᵥ | AlkL | E₄* | |
| 284 | E_{1b}E₂E₃Eᵢᵥ | AlkL | E₄* | |
| 285 | E₁ₐE₂EᵢᵥE₄ | AlkL | E₄* | |
| 286 | E_{1b}E₂EᵢᵥE₄ | AlkL | E₄* | |
| 287 | E₁ₐE₂E₃EᵢᵥE₄ | AlkL | E₄* | |
| 288 | E_{1b}E₂E₃EᵢᵥE₄ | AlkL | E₄* | |
| 289 | E₁ₐE₂ | AlkL | E₅ | |
| 290 | E_{1b}E₂ | AlkL | E₅ | |
| 291 | E₁ₐE₂E₃ | AlkL | E₅ | |
| 292 | E_{1b}E₂E₃ | AlkL | E₅ | |
| 293 | E₁ₐE₂Eᵢ | AlkL | E₅ | |
| 294 | E_{1b}E₂Eᵢ | AlkL | E₅ | |
| 295 | E₁ₐE₂Eᵢᵢ | AlkL | E₅ | |
| 296 | E_{1b}E₂Eᵢᵢ | AlkL | E₅ | |
| 297 | E₁ₐE₂EᵢᵢE_{iib} | AlkL | E₅ | |
| 298 | E_{1b}E₂EᵢᵢE_{iib} | AlkL | E₅ | |
| 299 | E₁ₐE₂E₃Eᵢ | AlkL | E₅ | |
| 300 | E_{1b}E₂E₃Eᵢ | AlkL | E₅ | |
| 301 | E₁ₐE₂E₃Eᵢᵢ | AlkL | E₅ | |
| 302 | E_{1b}E₂E₃Eᵢᵢ | AlkL | E₅ | |
| 303 | E₁ₐE₂E₃EᵢᵢE_{iib} | AlkL | E₅ | |
| 304 | E_{1b}E₂E₃EᵢᵢE_{iib} | AlkL | E₅ | |
| 305 | E₁ₐE₂E₄ | AlkL | E₅ | |
| 306 | E_{1b}E₂E₄ | AlkL | E₅ | |
| 307 | E₁ₐE₂E₃E₄ | AlkL | E₅ | |
| 308 | E_{1b}E₂E₃E₄ | AlkL | E₅ | |
| 309 | E₁ₐE₂EᵢE₄ | AlkL | E₅ | |
| 310 | E_{1b}E₂EᵢE₄ | AlkL | E₅ | |
| 311 | E₁ₐE₂EᵢᵢE₄ | AlkL | E₅ | |
| 312 | E_{1b}E₂EᵢᵢE₄ | AlkL | E₅ | |
| 313 | E₁ₐE₂EᵢᵢE_{iib}E₄ | AlkL | E₅ | |
| 314 | E_{1b}E₂EᵢᵢE_{iib}E₄ | AlkL | E₅ | |
| 315 | E₁ₐE₂E₃EᵢE₄ | AlkL | E₅ | |
| 316 | E_{1b}E₂E₃EᵢE₄ | AlkL | E₅ | |
| 317 | E₁ₐE₂E₃EᵢᵢE₄ | AlkL | E₅ | |
| 318 | E_{1b}E₂E₃EᵢᵢE₄ | AlkL | E₅ | |
| 319 | E₁ₐE₂E₃EᵢᵢE_{iib}E₄ | AlkL | E₅ | |
| 320 | E_{1b}E₂E₃EᵢᵢE_{iib}E₄ | AlkL | E₅ | |
| 321 | E₁ₐE₂Eᵢᵢᵢ | AlkL | E₅ | |
| 322 | E_{1b}E₂Eᵢᵢᵢ | AlkL | E₅ | |
| 323 | E₁ₐE₂E₃Eᵢᵢᵢ | AlkL | E₅ | |
| 324 | E_{1b}E₂E₃Eᵢᵢᵢ | AlkL | E₅ | |
| 325 | E₁ₐE₂EᵢᵢᵢE₄ | AlkL | E₅ | |
| 326 | E_{1b}E₂EᵢᵢᵢE₄ | AlkL | E₅ | |
| 327 | E₁ₐE₂E₃EᵢᵢᵢE₄ | AlkL | E₅ | |
| 328 | E_{1b}E₂E₃EᵢᵢᵢE₄ | AlkL | E₅ | |
| 329 | E₁ₐE₂Eᵢᵥ | AlkL | E₅ | |
| 330 | E_{1b}E₂Eᵢᵥ | AlkL | E₅ | |
| 331 | E₁ₐE₂E₃Eᵢᵥ | AlkL | E₅ | |
| 332 | E_{1b}E₂E₃Eᵢᵥ | AlkL | E₅ | |
| 333 | E₁ₐE₂EᵢᵥE₄ | AlkL | E₅ | |
| 334 | E_{1b}E₂EᵢᵥE₄ | AlkL | E₅ | |
| 335 | E₁ₐE₂E₃EᵢᵥE₄ | AlkL | E₅ | |
| 336 | E_{1b}E₂E₃EᵢᵥE₄ | AlkL | E₅ | |
| 337 | E₁ₐE₂ | AlkL | E₄*E₅ | |
| 338 | E_{1b}E₂ | AlkL | E₄*E₅ | |
| 339 | E₁ₐE₂E₃ | AlkL | E₄*E₅ | |
| 340 | E_{1b}E₂E₃ | AlkL | E₄*E₅ | |
| 341 | E₁ₐE₂Eᵢ | AlkL | E₄*E₅ | |
| 342 | E_{1b}E₂Eᵢ | AlkL | E₄*E₅ | |
| 343 | E₁ₐE₂Eᵢᵢ | AlkL | E₄*E₅ | |
| 344 | E_{1b}E₂Eᵢᵢ | AlkL | E₄*E₅ | |
| 345 | E₁ₐE₂EᵢᵢE_{iib} | AlkL | E₄*E₅ | |
| 346 | E_{1b}E₂EᵢᵢE_{iib} | AlkL | E₄*E₅ | |
| 347 | E₁ₐE₂E₃Eᵢ | AlkL | E₄*E₅ | |
| 348 | E_{1b}E₂E₃Eᵢ | AlkL | E₄*E₅ | |
| 349 | E₁ₐE₂E₃Eᵢᵢ | AlkL | E₄*E₅ | |
| 350 | E_{1b}E₂E₃Eᵢᵢ | AlkL | E₄*E₅ | |
| 351 | E₁ₐE₂E₃EᵢᵢE_{iib} | AlkL | E₄*E₅ | |
| 352 | E_{1b}E₂E₃EᵢᵢE_{iib} | AlkL | E₄*E₅ | |
| 353 | E₁ₐE₂E₄ | AlkL | E₄*E₅ | |
| 354 | E_{1b}E₂E₄ | AlkL | E₄*E₅ | |
| 355 | E₁ₐE₂E₃E₄ | AlkL | E₄*E₅ | |
| 356 | E_{1b}E₂E₃E₄ | AlkL | E₄*E₅ | |
| 357 | E₁ₐE₂EᵢE₄ | AlkL | E₄*E₅ | |
| 358 | E_{1b}E₂EᵢE₄ | AlkL | E₄*E₅ | |
| 359 | E₁ₐE₂EᵢᵢE₄ | AlkL | E₄*E₅ | |
| 360 | E_{1b}E₂EᵢᵢE₄ | AlkL | E₄*E₅ | |
| 361 | E₁ₐE₂EᵢᵢE_{iib}E₄ | AlkL | E₄*E₅ | |
| 362 | E_{1b}E₂EᵢᵢE_{iib}E₄ | AlkL | E₄*E₅ | |
| 363 | E₁ₐE₂E₃EᵢE₄ | AlkL | E₄*E₅ | |
| 364 | E_{1b}E₂E₃EᵢE₄ | AlkL | E₄*E₅ | |
| 365 | E₁ₐE₂E₃EᵢᵢE₄ | AlkL | E₄*E₅ | |
| 366 | E_{1b}E₂E₃EᵢᵢE₄ | AlkL | E₄*E₅ | |
| 367 | E₁ₐE₂E₃EᵢᵢE_{iib}E₄ | AlkL | E₄*E₅ | |
| 368 | E_{1b}E₂E₃EᵢᵢE_{iib}E₄ | AlkL | E₄*E₅ | |
| 369 | E₁ₐE₂Eᵢᵢᵢ | AlkL | E₄*E₅ | |
| 370 | E_{1b}E₂Eᵢᵢᵢ | AlkL | E₄*E₅ | |
| 371 | E₁ₐE₂E₃Eᵢᵢᵢ | AlkL | E₄*E₅ | |
| 372 | E_{1b}E₂E₃Eᵢᵢᵢ | AlkL | E₄*E₅ | |
| 373 | E₁ₐE₂EᵢᵢᵢE₄ | AlkL | E₄*E₅ | |
| 374 | E_{1b}E₂EᵢᵢᵢE₄ | AlkL | E₄*E₅ | |
| 375 | E₁ₐE₂E₃EᵢᵢᵢE₄ | AlkL | E₄*E₅ | |
| 376 | E_{1b}E₂E₃EᵢᵢᵢE₄ | AlkL | E₄*E₅ | |
| 377 | E₁ₐE₂Eᵢᵥ | AlkL | E₄*E₅ | |
| 378 | E_{1b}E₂Eᵢᵥ | AlkL | E₄*E₅ | |
| 379 | E₁ₐE₂E₃Eᵢᵥ | AlkL | E₄*E₅ | |
| 380 | E_{1b}E₂E₃Eᵢᵥ | AlkL | E₄*E₅ | |
| 381 | E₁ₐE₂EᵢᵥE₄ | AlkL | E₄*E₅ | |
| 382 | E_{1b}E₂EᵢᵥE₄ | AlkL | E₄*E₅ | |
| 383 | E₁ₐE₂E₃EᵢᵥE₄ | AlkL | E₄*E₅ | |
| 384 | E_{1b}E₂E₃EᵢᵥE₄ | AlkL | E₄*E₅ | |
| 385 | E₁ₐE₂ | | | Eₐ |
| 386 | E_{1b}E₂ | | | Eₐ |
| 387 | E₁ₐE₂E₃ | | | Eₐ |
| 388 | E_{1b}E₂E₃ | | | Eₐ |
| 389 | E₁ₐE₂Eᵢ | | | Eₐ |
| 390 | E_{1b}E₂Eᵢ | | | Eₐ |
| 391 | E₁ₐE₂Eᵢᵢ | | | Eₐ |
| 392 | E_{1b}E₂Eᵢᵢ | | | Eₐ |
| 393 | E₁ₐE₂EᵢᵢE_{iib} | | | Eₐ |
| 394 | E_{1b}E₂EᵢᵢE_{iib} | | | Eₐ |
| 395 | E₁ₐE₂E₃Eᵢ | | | Eₐ |
| 396 | E_{1b}E₂E₃Eᵢ | | | Eₐ |
| 397 | E₁ₐE₂E₃Eᵢᵢ | | | Eₐ |
| 398 | E_{1b}E₂E₃Eᵢᵢ | | | Eₐ |
| 399 | E₁ₐE₂E₃EᵢᵢE_{iib} | | | Eₐ |
| 400 | E_{1b}E₂E₃EᵢᵢE_{iib} | | | Eₐ |
| 401 | E₁ₐE₂E₄ | | | Eₐ |
| 402 | E_{1b}E₂E₄ | | | Eₐ |
| 403 | E₁ₐE₂E₃E₄ | | | Eₐ |
| 404 | E_{1b}E₂E₃E₄ | | | Eₐ |
| 405 | E₁ₐE₂EᵢE₄ | | | Eₐ |
| 406 | E_{1b}E₂EᵢE₄ | | | Eₐ |
| 407 | E₁ₐE₂EᵢᵢE₄ | | | Eₐ |
| 408 | E_{1b}E₂EᵢᵢE₄ | | | Eₐ |
| 409 | E₁ₐE₂EᵢᵢE_{iib}E₄ | | | Eₐ |
| 410 | E_{1b}E₂EᵢᵢE_{iib}E₄ | | | Eₐ |
| 411 | E₁ₐE₂E₃EᵢE₄ | | | Eₐ |
| 412 | E_{1b}E₂E₃EᵢE₄ | | | Eₐ |
| 413 | E₁ₐE₂E₃EᵢᵢE₄ | | | Eₐ |
| 414 | E_{1b}E₂E₃EᵢᵢE₄ | | | Eₐ |
| 415 | E₁ₐE₂E₃EᵢᵢE_{iib}E₄ | | | Eₐ |
| 416 | E_{1b}E₂E₃EᵢᵢE_{iib}E₄ | | | Eₐ |
| 417 | E₁ₐE₂Eᵢᵢᵢ | | | Eₐ |
| 418 | E_{1b}E₂Eᵢᵢᵢ | | | Eₐ |
| 419 | E₁ₐE₂E₃Eᵢᵢᵢ | | | Eₐ |
| 420 | E_{1b}E₂E₃Eᵢᵢᵢ | | | Eₐ |
| 421 | E₁ₐE₂EᵢᵢᵢE₄ | | | Eₐ |
| 422 | E_{1b}E₂EᵢᵢᵢE₄ | | | Eₐ |
| 423 | E₁ₐE₂E₃EᵢᵢᵢE₄ | | | Eₐ |
| 424 | E_{1b}E₂E₃EᵢᵢᵢE₄ | | | Eₐ |
| 425 | E₁ₐE₂Eᵢᵥ | | | Eₐ |
| 426 | E_{1b}E₂Eᵢᵥ | | | Eₐ |
| 427 | E₁ₐE₂E₃Eᵢᵥ | | | Eₐ |
| 428 | E_{1b}E₂E₃Eᵢᵥ | | | Eₐ |
| 429 | E₁ₐE₂EᵢᵥE₄ | | | Eₐ |
| 430 | E_{1b}E₂EᵢᵥE₄ | | | Eₐ |
| 431 | E₁ₐE₂E₃EᵢᵥE₄ | | | Eₐ |
| 432 | E_{1b}E₂E₃EᵢᵥE₄ | | | Eₐ |
| 433 | E₁ₐE₂ | | E₄* | Eₐ |
| 434 | E_{1b}E₂ | | E₄* | Eₐ |
| 435 | E₁ₐE₂E₃ | | E₄* | Eₐ |
| 436 | E_{1b}E₂E₃ | | E₄* | Eₐ |
| 437 | E₁ₐE₂Eᵢ | | E₄* | Eₐ |
| 438 | E_{1b}E₂Eᵢ | | E₄* | Eₐ |
| 439 | E₁ₐE₂Eᵢᵢ | | E₄* | Eₐ |
| 440 | E_{1b}E₂Eᵢᵢ | | E₄* | Eₐ |
| 441 | E₁ₐE₂EᵢᵢE_{iib} | | E₄* | Eₐ |
| 442 | E_{1b}E₂EᵢᵢE_{iib} | | E₄* | Eₐ |
| 443 | E₁ₐE₂E₃Eᵢ | | E₄* | Eₐ |
| 444 | E_{1b}E₂E₃Eᵢ | | E₄* | Eₐ |
| 445 | E₁ₐE₂E₃Eᵢᵢ | | E₄* | Eₐ |
| 446 | E_{1b}E₂E₃Eᵢᵢ | | E₄* | Eₐ |
| 447 | E₁ₐE₂E₃EᵢᵢE_{iib} | | E₄* | Eₐ |
| 448 | E_{1b}E₂E₃EᵢᵢE_{iib} | | E₄* | Eₐ |
| 449 | E₁ₐE₂E₄ | | E₄* | Eₐ |
| 450 | E_{1b}E₂E₄ | | E₄* | Eₐ |
| 451 | E₁ₐE₂E₃E₄ | | E₄* | Eₐ |
| 452 | E_{1b}E₂E₃E₄ | | E₄* | Eₐ |
| 453 | E₁ₐE₂EᵢE₄ | | E₄* | Eₐ |
| 454 | E_{1b}E₂EᵢE₄ | | E₄* | Eₐ |
| 455 | E₁ₐE₂EᵢᵢE₄ | | E₄* | Eₐ |
| 456 | E_{1b}E₂EᵢᵢE₄ | | E₄* | Eₐ |
| 457 | E₁ₐE₂EᵢᵢE_{iib}E₄ | | E₄* | Eₐ |
| 458 | E_{1b}E₂EᵢᵢE_{iib}E₄ | | E₄* | Eₐ |
| 459 | E₁ₐE₂E₃EᵢE₄ | | E₄* | Eₐ |
| 460 | E_{1b}E₂E₃EᵢE₄ | | E₄* | Eₐ |
| 461 | E₁ₐE₂E₃EᵢᵢE₄ | | E₄* | Eₐ |
| 462 | E_{1b}E₂E₃EᵢᵢE₄ | | E₄* | Eₐ |
| 463 | E₁ₐE₂E₃EᵢᵢE_{iib}E₄ | | E₄* | Eₐ |
| 464 | E_{1b}E₂E₃EᵢᵢE_{iib}E₄ | | E₄* | Eₐ |
| 465 | E₁ₐE₂Eᵢᵢᵢ | | E₄* | Eₐ |
| 466 | E_{1b}E₂Eᵢᵢᵢ | | E₄* | Eₐ |
| 467 | E₁ₐE₂E₃Eᵢᵢᵢ | | E₄* | Eₐ |
| 468 | E_{1b}E₂E₃Eᵢᵢᵢ | | E₄* | Eₐ |
| 469 | E₁ₐE₂EᵢᵢᵢE₄ | | E₄* | Eₐ |
| 470 | E_{1b}E₂EᵢᵢᵢE₄ | | E₄* | Eₐ |
| 471 | E₁ₐE₂E₃EᵢᵢᵢE₄ | | E₄* | Eₐ |
| 472 | E_{1b}E₂E₃EᵢᵢᵢE₄ | | E₄* | Eₐ |
| 473 | E₁ₐE₂Eᵢᵥ | | E₄* | Eₐ |
| 474 | E_{1b}E₂Eᵢᵥ | | E₄* | Eₐ |
| 475 | E₁ₐE₂E₃Eᵢᵥ | | E₄* | Eₐ |
| 476 | E_{1b}E₂E₃Eᵢᵥ | | E₄* | Eₐ |
| 477 | E₁ₐE₂EᵢᵥE₄ | | E₄* | Eₐ |
| 478 | E_{1b}E₂EᵢᵥE₄ | | E₄* | Eₐ |
| 479 | E₁ₐE₂E₃EᵢᵥE₄ | | E₄* | Eₐ |
| 480 | E_{1b}E₂E₃EᵢᵥE₄ | | E₄* | Eₐ |
| 481 | E₁ₐE₂ | | E₅ | Eₐ |
| 482 | E_{1b}E₂ | | E₅ | Eₐ |
| 483 | E₁ₐE₂E₃ | | E₅ | Eₐ |
| 484 | E_{1b}E₂E₃ | | E₅ | Eₐ |
| 485 | E₁ₐE₂Eᵢ | | E₅ | Eₐ |
| 486 | E_{1b}E₂Eᵢ | | E₅ | Eₐ |
| 487 | E₁ₐE₂Eᵢᵢ | | E₅ | Eₐ |
| 488 | E_{1b}E₂Eᵢᵢ | | E₅ | Eₐ |
| 489 | E₁ₐE₂EᵢᵢE_{iib} | | E₅ | Eₐ |
| 490 | E_{1b}E₂EᵢᵢE_{iib} | | E₅ | Eₐ |
| 491 | E₁ₐE₂E₃Eᵢ | | E₅ | Eₐ |
| 492 | E_{1b}E₂E₃Eᵢ | | E₅ | Eₐ |
| 493 | E₁ₐE₂E₃Eᵢᵢ | | E₅ | Eₐ |
| 494 | E_{1b}E₂E₃Eᵢᵢ | | E₅ | Eₐ |
| 495 | E₁ₐE₂E₃EᵢᵢE_{iib} | | E₅ | Eₐ |
| 496 | E_{1b}E₂E₃EᵢᵢE_{iib} | | E₅ | Eₐ |
| 497 | E₁ₐE₂E₄ | | E₅ | Eₐ |
| 498 | E_{1b}E₂E₄ | | E₅ | Eₐ |
| 499 | E₁ₐE₂E₃E₄ | | E₅ | Eₐ |
| 500 | E_{1b}E₂E₃E₄ | | E₅ | Eₐ |
| 501 | E₁ₐE₂EᵢE₄ | | E₅ | Eₐ |
| 502 | E_{1b}E₂EᵢE₄ | | E₅ | Eₐ |
| 503 | E₁ₐE₂EᵢᵢE₄ | | E₅ | Eₐ |
| 504 | E_{1b}E₂EᵢᵢE₄ | | E₅ | Eₐ |
| 505 | E₁ₐE₂EᵢᵢE_{iib}E₄ | | E₅ | Eₐ |
| 506 | E_{1b}E₂EᵢᵢE_{iib}E₄ | | E₅ | Eₐ |
| 507 | E₁ₐE₂E₃EᵢE₄ | | E₅ | Eₐ |
| 508 | E_{1b}E₂E₃EᵢE₄ | | E₅ | Eₐ |
| 509 | E₁ₐE₂E₃EᵢᵢE₄ | | E₅ | Eₐ |
| 510 | E_{1b}E₂E₃EᵢᵢE₄ | | E₅ | Eₐ |
| 511 | E₁ₐE₂E₃EᵢᵢE_{iib}E₄ | | E₅ | Eₐ |
| 512 | E_{1b}E₂E₃EᵢᵢE_{iib}E₄ | | E₅ | Eₐ |
| 513 | E₁ₐE₂Eᵢᵢᵢ | | E₅ | Eₐ |
| 514 | E_{1b}E₂Eᵢᵢᵢ | | E₅ | Eₐ |
| 515 | E₁ₐE₂E₃Eᵢᵢᵢ | | E₅ | Eₐ |
| 516 | E_{1b}E₂E₃Eᵢᵢᵢ | | E₅ | Eₐ |
| 517 | E₁ₐE₂EᵢᵢᵢE₄ | | E₅ | Eₐ |
| 518 | E_{1b}E₂EᵢᵢᵢE₄ | | E₅ | Eₐ |
| 519 | E₁ₐE₂E₃EᵢᵢᵢE₄ | | E₅ | Eₐ |
| 520 | E_{1b}E₂E₃EᵢᵢᵢE₄ | | E₅ | Eₐ |
| 521 | E₁ₐE₂Eᵢᵥ | | E₅ | Eₐ |
| 522 | E_{1b}E₂Eᵢᵥ | | E₅ | Eₐ |
| 523 | E₁ₐE₂E₃Eᵢᵥ | | E₅ | Eₐ |
| 524 | E_{1b}E₂E₃Eᵢᵥ | | E₅ | Eₐ |
| 525 | E₁ₐE₂EᵢᵥE₄ | | E₅ | Eₐ |
| 526 | E_{1b}E₂EᵢᵥE₄ | | E₅ | Eₐ |
| 527 | E₁ₐE₂E₃EᵢᵥE₄ | | E₅ | Eₐ |
| 528 | E_{1b}E₂E₃EᵢᵥE₄ | | E₅ | Eₐ |
| 529 | E₁ₐE₂ | | E₄*E₅ | Eₐ |
| 530 | E_{1b}E₂ | | E₄*E₅ | Eₐ |
| 531 | E₁ₐE₂E₃ | | E₄*E₅ | Eₐ |
| 532 | E_{1b}E₂E₃ | | E₄*E₅ | Eₐ |
| 533 | E₁ₐE₂Eᵢ | | E₄*E₅ | Eₐ |
| 534 | E_{1b}E₂Eᵢ | | E₄*E₅ | Eₐ |
| 535 | E₁ₐE₂Eᵢᵢ | | E₄*E₅ | Eₐ |
| 536 | E_{1b}E₂Eᵢᵢ | | E₄*E₅ | Eₐ |
| 537 | E₁ₐE₂EᵢᵢE_{iib} | | E₄*E₅ | Eₐ |
| 538 | E_{1b}E₂EᵢᵢE_{iib} | | E₄*E₅ | Eₐ |
| 539 | E₁ₐE₂E₃Eᵢ | | E₄*E₅ | Eₐ |
| 540 | E_{1b}E₂E₃Eᵢ | | E₄*E₅ | Eₐ |
| 541 | E₁ₐE₂E₃Eᵢᵢ | | E₄*E₅ | Eₐ |
| 542 | E_{1b}E₂E₃Eᵢᵢ | | E₄*E₅ | Eₐ |
| 543 | E₁ₐE₂E₃EᵢᵢE_{iib} | | E₄*E₅ | Eₐ |
| 544 | E_{1b}E₂E₃EᵢᵢE_{iib} | | E₄*E₅ | Eₐ |
| 545 | E₁ₐE₂E₄ | | E₄*E₅ | Eₐ |
| 546 | E_{1b}E₂E₄ | | E₄*E₅ | Eₐ |
| 547 | E₁ₐE₂E₃E₄ | | E₄*E₅ | Eₐ |
| 548 | E_{1b}E₂E₃E₄ | | E₄*E₅ | Eₐ |
| 549 | E₁ₐE₂EᵢE₄ | | E₄*E₅ | Eₐ |
| 550 | E_{1b}E₂EᵢE₄ | | E₄*E₅ | Eₐ |
| 551 | E₁ₐE₂EᵢᵢE₄ | | E₄*E₅ | Eₐ |
| 552 | E_{1b}E₂EᵢᵢE₄ | | E₄*E₅ | Eₐ |
| 553 | E₁ₐE₂EᵢᵢE_{iib}E₄ | | E₄*E₅ | Eₐ |
| 554 | E_{1b}E₂EᵢᵢE_{iib}E₄ | | E₄*E₅ | Eₐ |
| 555 | E₁ₐE₂E₃EᵢE₄ | | E₄*E₅ | Eₐ |
| 556 | E_{1b}E₂E₃EᵢE₄ | | E₄*E₅ | Eₐ |
| 557 | E₁ₐE₂E₃EᵢᵢE₄ | | E₄*E₅ | Eₐ |
| 558 | E_{1b}E₂E₃EᵢᵢE₄ | | E₄*E₅ | Eₐ |
| 559 | E₁ₐE₂E₃EᵢᵢE_{iib}E₄ | | E₄*E₅ | Eₐ |
| 560 | E_{1b}E₂E₃EᵢᵢE_{iib}E₄ | | E₄*E₅ | Eₐ |
| 561 | E₁ₐE₂Eᵢᵢᵢ | | E₄*E₅ | Eₐ |
| 562 | E_{1b}E₂Eᵢᵢᵢ | | E₄*E₅ | Eₐ |
| 563 | E₁ₐE₂E₃Eᵢᵢᵢ | | E₄*E₅ | Eₐ |
| 564 | E_{1b}E₂E₃Eᵢᵢᵢ | | E₄*E₅ | Eₐ |
| 565 | E₁ₐE₂EᵢᵢᵢE₄ | | E₄*E₅ | Eₐ |
| 566 | E_{1b}E₂EᵢᵢᵢE₄ | | E₄*E₅ | Eₐ |
| 567 | E₁ₐE₂E₃EᵢᵢᵢE₄ | | E₄*E₅ | Eₐ |
| 568 | E_{1b}E₂E₃EᵢᵢᵢE₄ | | E₄*E₅ | Eₐ |
| 569 | E₁ₐE₂Eᵢᵥ | | E₄*E₅ | Eₐ |
| 570 | E_{1b}E₂Eᵢᵥ | | E₄*E₅ | Eₐ |
| 571 | E₁ₐE₂E₃Eᵢᵥ | | E₄*E₅ | Eₐ |
| 572 | E_{1b}E₂E₃Eᵢᵥ | | E₄*E₅ | Eₐ |
| 573 | E₁ₐE₂EᵢᵥE₄ | | E₄*E₅ | Eₐ |
| 574 | E_{1b}E₂EᵢᵥE₄ | | E₄*E₅ | Eₐ |
| 575 | E₁ₐE₂E₃EᵢᵥE₄ | | E₄*E₅ | Eₐ |
| 576 | E_{1b}E₂E₃EᵢᵥE₄ | | E₄*E₅ | Eₐ |
| 577 | E₁ₐE₂ | AlkL | | Eₐ |
| 578 | E_{1b}E₂ | AlkL | | Eₐ |
| 579 | E₁ₐE₂E₃ | AlkL | | Eₐ |
| 580 | E_{1b}E₂E₃ | AlkL | | Eₐ |
| 581 | E₁ₐE₂Eᵢ | AlkL | | Eₐ |
| 582 | E_{1b}E₂Eᵢ | AlkL | | Eₐ |
| 583 | E₁ₐE₂Eᵢᵢ | AlkL | | Eₐ |
| 584 | E_{1b}E₂Eᵢᵢ | AlkL | | Eₐ |
| 585 | E₁ₐE₂EᵢᵢE_{iib} | AlkL | | Eₐ |
| 586 | E_{1b}E₂EᵢᵢE_{iib} | AlkL | | Eₐ |
| 587 | E₁ₐE₂E₃Eᵢ | AlkL | | Eₐ |
| 588 | E_{1b}E₂E₃Eᵢ | AlkL | | Eₐ |
| 589 | E₁ₐE₂E₃Eᵢᵢ | AlkL | | Eₐ |
| 590 | E_{1b}E₂E₃Eᵢᵢ | AlkL | | Eₐ |
| 591 | E₁ₐE₂E₃EᵢᵢE_{iib} | AlkL | | Eₐ |
| 592 | E_{1b}E₂E₃EᵢᵢE_{iib} | AlkL | | Eₐ |
| 593 | E₁ₐE₂E₄ | AlkL | | Eₐ |
| 594 | E_{1b}E₂E₄ | AlkL | | Eₐ |
| 595 | E₁ₐE₂E₃E₄ | AlkL | | Eₐ |
| 596 | E_{1b}E₂E₃E₄ | AlkL | | Eₐ |
| 597 | E₁ₐE₂EᵢE₄ | AlkL | | Eₐ |
| 598 | E_{1b}E₂EᵢE₄ | AlkL | | Eₐ |
| 599 | E₁ₐE₂EᵢᵢE₄ | AlkL | | Eₐ |
| 600 | E_{1b}E₂EᵢᵢE₄ | AlkL | | Eₐ |
| 601 | E₁ₐE₂EᵢᵢE_{iib}E₄ | AlkL | | Eₐ |
| 602 | E_{1b}E₂EᵢᵢE_{iib}E₄ | AlkL | | Eₐ |
| 603 | E₁ₐE₂E₃EᵢE₄ | AlkL | | Eₐ |
| 604 | E_{1b}E₂E₃EᵢE₄ | AlkL | | Eₐ |
| 605 | E₁ₐE₂E₃EᵢᵢE₄ | AlkL | | Eₐ |
| 606 | E_{1b}E₂E₃EᵢᵢE₄ | AlkL | | Eₐ |
| 607 | E₁ₐE₂E₃EᵢᵢE_{iib}E₄ | AlkL | | Eₐ |
| 608 | E_{1b}E₂E₃EᵢᵢE_{iib}E₄ | AlkL | | Eₐ |
| 609 | E₁ₐE₂Eᵢᵢᵢ | AlkL | | Eₐ |
| 610 | E_{1b}E₂Eᵢᵢᵢ | AlkL | | Eₐ |
| 611 | E₁ₐE₂E₃Eᵢᵢᵢ | AlkL | | Eₐ |
| 612 | E_{1b}E₂E₃Eᵢᵢᵢ | AlkL | | Eₐ |
| 613 | E₁ₐE₂EᵢᵢᵢE₄ | AlkL | | Eₐ |
| 614 | E_{1b}E₂EᵢᵢᵢE₄ | AlkL | | Eₐ |
| 615 | E₁ₐE₂E₃EᵢᵢᵢE₄ | AlkL | | Eₐ |
| 616 | E_{1b}E₂E₃EᵢᵢᵢE₄ | AlkL | | Eₐ |
| 617 | E₁ₐE₂Eᵢᵥ | AlkL | | Eₐ |
| 618 | E_{1b}E₂Eᵢᵥ | AlkL | | Eₐ |
| 619 | E₁ₐE₂E₃Eᵢᵥ | AlkL | | Eₐ |
| 620 | E_{1b}E₂E₃Eᵢᵥ | AlkL | | Eₐ |
| 621 | E₁ₐE₂EᵢᵥE₄ | AlkL | | Eₐ |
| 622 | E_{1b}E₂EᵢᵥE₄ | AlkL | | Eₐ |
| 623 | E₁ₐE₂E₃EᵢᵥE₄ | AlkL | | Eₐ |
| 624 | E_{1b}E₂E₃EᵢᵥE₄ | AlkL | | Eₐ |
| 625 | E₁ₐE₂ | AlkL | E₄* | Eₐ |
| 626 | E_{1b}E₂ | AlkL | E₄* | Eₐ |
| 627 | E₁ₐE₂E₃ | AlkL | E₄* | Eₐ |
| 628 | E_{1b}E₂E₃ | AlkL | E₄* | Eₐ |
| 629 | E₁ₐE₂Eᵢ | AlkL | E₄* | Eₐ |
| 630 | E_{1b}E₂Eᵢ | AlkL | E₄* | Eₐ |
| 631 | E₁ₐE₂Eᵢᵢ | AlkL | E₄* | Eₐ |
| 632 | E_{1b}E₂Eᵢᵢ | AlkL | E₄* | Eₐ |
| 633 | E₁ₐE₂EᵢᵢE_{iib} | AlkL | E₄* | Eₐ |
| 634 | E_{1b}E₂EᵢᵢE_{iib} | AlkL | E₄* | Eₐ |
| 635 | E₁ₐE₂E₃Eᵢ | AlkL | E₄* | Eₐ |
| 636 | E_{1b}E₂E₃Eᵢ | AlkL | E₄* | Eₐ |
| 637 | E₁ₐE₂E₃Eᵢᵢ | AlkL | E₄* | Eₐ |
| 638 | E_{1b}E₂E₃Eᵢᵢ | AlkL | E₄* | Eₐ |
| 639 | E₁ₐE₂E₃EᵢᵢE_{iib} | AlkL | E₄* | Eₐ |
| 640 | E_{1b}E₂E₃EᵢᵢE_{iib} | AlkL | E₄* | Eₐ |
| 641 | E₁ₐE₂E₄ | AlkL | E₄* | Eₐ |
| 642 | E_{1b}E₂E₄ | AlkL | E₄* | Eₐ |
| 643 | E₁ₐE₂E₃E₄ | AlkL | E₄* | Eₐ |
| 644 | E_{1b}E₂E₃E₄ | AlkL | E₄* | Eₐ |
| 645 | E₁ₐE₂EᵢE₄ | AlkL | E₄* | Eₐ |
| 646 | E_{1b}E₂EᵢE₄ | AlkL | E₄* | Eₐ |
| 647 | E₁ₐE₂EᵢᵢE₄ | AlkL | E₄* | Eₐ |
| 648 | E_{1b}E₂EᵢᵢE₄ | AlkL | E₄* | Eₐ |
| 649 | E₁ₐE₂EᵢᵢE_{iib}E₄ | AlkL | E₄* | Eₐ |
| 650 | E_{1b}E₂EᵢᵢE_{iib}E₄ | AlkL | E₄* | Eₐ |
| 651 | E₁ₐE₂E₃EᵢE₄ | AlkL | E₄* | Eₐ |
| 652 | E_{1b}E₂E₃EᵢE₄ | AlkL | E₄* | Eₐ |
| 653 | E₁ₐE₂E₃EᵢᵢE₄ | AlkL | E₄* | Eₐ |
| 654 | E_{1b}E₂E₃EᵢᵢE₄ | AlkL | E₄* | Eₐ |
| 655 | E₁ₐE₂E₃EᵢᵢE_{iib}E₄ | AlkL | E₄* | Eₐ |
| 656 | E_{1b}E₂E₃EᵢᵢE_{iib}E₄ | AlkL | E₄* | Eₐ |
| 657 | E₁ₐE₂Eᵢᵢᵢ | AlkL | E₄* | Eₐ |
| 658 | E_{1b}E₂Eᵢᵢᵢ | AlkL | E₄* | Eₐ |
| 659 | E₁ₐE₂E₃Eᵢᵢᵢ | AlkL | E₄* | Eₐ |
| 660 | E_{1b}E₂E₃Eᵢᵢᵢ | AlkL | E₄* | Eₐ |
| 661 | E₁ₐE₂EᵢᵢᵢE₄ | AlkL | E₄* | Eₐ |
| 662 | E_{1b}E₂EᵢᵢᵢE₄ | AlkL | E₄* | Eₐ |
| 663 | E₁ₐE₂E₃EᵢᵢᵢE₄ | AlkL | E₄* | Eₐ |
| 664 | E_{1b}E₂E₃EᵢᵢᵢE₄ | AlkL | E₄* | Eₐ |
| 665 | E₁ₐE₂Eᵢᵥ | AlkL | E₄* | Eₐ |
| 666 | E_{1b}E₂Eᵢᵥ | AlkL | E₄* | Eₐ |
| 667 | E₁ₐE₂E₃Eᵢᵥ | AlkL | E₄* | Eₐ |
| 668 | E_{1b}E₂E₃Eᵢᵥ | AlkL | E₄* | Eₐ |
| 669 | E₁ₐE₂EᵢᵥE₄ | AlkL | E₄* | Eₐ |
| 670 | E_{1b}E₂EᵢᵥE₄ | AlkL | E₄* | Eₐ |
| 671 | E₁ₐE₂E₃EᵢᵥE₄ | AlkL | E₄* | Eₐ |
| 672 | E_{1b}E₂E₃EᵢᵥE₄ | AlkL | E₄* | Eₐ |
| 673 | E₁ₐE₂ | AlkL | E₅ | Eₐ |
| 674 | E_{1b}E₂ | AlkL | E₅ | Eₐ |
| 675 | E₁ₐE₂E₃ | AlkL | E₅ | Eₐ |
| 676 | E_{1b}E₂E₃ | AlkL | E₅ | Eₐ |
| 677 | E₁ₐE₂Eᵢ | AlkL | E₅ | Eₐ |
| 678 | E_{1b}E₂Eᵢ | AlkL | E₅ | Eₐ |
| 679 | E₁ₐE₂Eᵢᵢ | AlkL | E₅ | Eₐ |
| 680 | E_{1b}E₂Eᵢᵢ | AlkL | E₅ | Eₐ |
| 681 | E₁ₐE₂EᵢᵢE_{iib} | AlkL | E₅ | Eₐ |
| 682 | E_{1b}E₂EᵢᵢE_{iib} | AlkL | E₅ | Eₐ |
| 683 | E₁ₐE₂E₃Eᵢ | AlkL | E₅ | Eₐ |
| 684 | E_{1b}E₂E₃Eᵢ | AlkL | E₅ | Eₐ |
| 685 | E₁ₐE₂E₃Eᵢᵢ | AlkL | E₅ | Eₐ |
| 686 | E_{1b}E₂E₃Eᵢᵢ | AlkL | E₅ | Eₐ |
| 687 | E₁ₐE₂E₃EᵢᵢE_{iib} | AlkL | E₅ | Eₐ |
| 688 | E_{1b}E₂E₃EᵢᵢE_{iib} | AlkL | E₅ | Eₐ |
| 689 | E₁ₐE₂E₄ | AlkL | E₅ | Eₐ |
| 690 | E_{1b}E₂E₄ | AlkL | E₅ | Eₐ |
| 691 | E₁ₐE₂E₃E₄ | AlkL | E₅ | Eₐ |
| 692 | E_{1b}E₂E₃E₄ | AlkL | E₅ | Eₐ |
| 693 | E₁ₐE₂EᵢE₄ | AlkL | E₅ | Eₐ |
| 694 | E_{1b}E₂EᵢE₄ | AlkL | E₅ | Eₐ |
| 695 | E₁ₐE₂EᵢᵢE₄ | AlkL | E₅ | Eₐ |
| 696 | E_{1b}E₂EᵢᵢE₄ | AlkL | E₅ | Eₐ |
| 697 | E₁ₐE₂EᵢᵢE_{iib}E₄ | AlkL | E₅ | Eₐ |
| 698 | E_{1b}E₂EᵢᵢE_{iib}E₄ | AlkL | E₅ | Eₐ |
| 699 | E₁ₐE₂E₃EᵢE₄ | AlkL | E₅ | Eₐ |
| 700 | E_{1b}E₂E₃EᵢE₄ | AlkL | E₅ | Eₐ |
| 701 | E₁ₐE₂E₃EᵢᵢE₄ | AlkL | E₅ | Eₐ |
| 702 | E_{1b}E₂E₃EᵢᵢE₄ | AlkL | E₅ | Eₐ |
| 703 | E₁ₐE₂E₃EᵢᵢE_{iib}E₄ | AlkL | E₅ | Eₐ |
| 704 | E_{1b}E₂E₃EᵢᵢE_{iib}E₄ | AlkL | E₅ | Eₐ |
| 705 | E₁ₐE₂Eᵢᵢᵢ | AlkL | E₅ | Eₐ |
| 706 | E_{1b}E₂Eᵢᵢᵢ | AlkL | E₅ | Eₐ |
| 707 | E₁ₐE₂E₃Eᵢᵢᵢ | AlkL | E₅ | Eₐ |
| 708 | E_{1b}E₂E₃Eᵢᵢᵢ | AlkL | E₅ | Eₐ |
| 709 | E₁ₐE₂EᵢᵢᵢE₄ | AlkL | E₅ | Eₐ |
| 710 | E_{1b}E₂EᵢᵢᵢE₄ | AlkL | E₅ | Eₐ |
| 711 | E₁ₐE₂E₃EᵢᵢᵢE₄ | AlkL | E₅ | Eₐ |
| 712 | E_{1b}E₂E₃EᵢᵢᵢE₄ | AlkL | E₅ | Eₐ |
| 713 | E₁ₐE₂Eᵢᵥ | AlkL | E₅ | Eₐ |
| 714 | E_{1b}E₂Eᵢᵥ | AlkL | E₅ | Eₐ |
| 715 | E₁ₐE₂E₃Eᵢᵥ | AlkL | E₅ | Eₐ |
| 716 | E_{1b}E₂E₃Eᵢᵥ | AlkL | E₅ | Eₐ |
| 717 | E₁ₐE₂EᵢᵥE₄ | AlkL | E₅ | Eₐ |
| 718 | E_{1b}E₂EᵢᵥE₄ | AlkL | E₅ | Eₐ |
| 719 | E₁ₐE₂E₃EᵢᵥE₄ | AlkL | E₅ | Eₐ |
| 720 | E_{1b}E₂E₃EᵢᵥE₄ | AlkL | E₅ | Eₐ |
| 721 | E₁ₐE₂ | AlkL | E₄*E₅ | Eₐ |
| 722 | E_{1b}E₂ | AlkL | E₄*E₅ | Eₐ |
| 723 | E₁ₐE₂E₃ | AlkL | E₄*E₅ | Eₐ |
| 724 | E_{1b}E₂E₃ | AlkL | E₄*E₅ | Eₐ |
| 725 | E₁ₐE₂Eᵢ | AlkL | E₄*E₅ | Eₐ |
| 726 | E_{1b}E₂Eᵢ | AlkL | E₄*E₅ | Eₐ |
| 727 | E₁ₐE₂Eᵢᵢ | AlkL | E₄*E₅ | Eₐ |
| 728 | E_{1b}E₂Eᵢᵢ | AlkL | E₄*E₅ | Eₐ |
| 729 | E₁ₐE₂EᵢᵢE_{iib} | AlkL | E₄*E₅ | Eₐ |
| 730 | E_{1b}E₂EᵢᵢE_{iib} | AlkL | E₄*E₅ | Eₐ |
| 731 | E₁ₐE₂E₃Eᵢ | AlkL | E₄*E₅ | Eₐ |
| 732 | E_{1b}E₂E₃Eᵢ | AlkL | E₄*E₅ | Eₐ |
| 733 | E₁ₐE₂E₃Eᵢᵢ | AlkL | E₄*E₅ | Eₐ |
| 734 | E_{1b}E₂E₃Eᵢᵢ | AlkL | E₄*E₅ | Eₐ |
| 735 | E₁ₐE₂E₃EᵢᵢE_{iib} | AlkL | E₄*E₅ | Eₐ |
| 736 | E_{1b}E₂E₃EᵢᵢE_{iib} | AlkL | E₄*E₅ | Eₐ |
| 737 | E₁ₐE₂E₄ | AlkL | E₄*E₅ | Eₐ |
| 738 | E_{1b}E₂E₄ | AlkL | E₄*E₅ | Eₐ |
| 739 | E₁ₐE₂E₃E₄ | AlkL | E₄*E₅ | Eₐ |
| 740 | E_{1b}E₂E₃E₄ | AlkL | E₄*E₅ | Eₐ |
| 741 | E₁ₐE₂EᵢE₄ | AlkL | E₄*E₅ | Eₐ |
| 742 | E_{1b}E₂EᵢE₄ | AlkL | E₄*E₅ | Eₐ |
| 743 | E₁ₐE₂EᵢᵢE₄ | AlkL | E₄*E₅ | Eₐ |
| 744 | E_{1b}E₂EᵢᵢE₄ | AlkL | E₄*E₅ | Eₐ |
| 745 | E₁ₐE₂EᵢᵢE_{iib}E₄ | AlkL | E₄*E₅ | Eₐ |
| 746 | E_{1b}E₂EᵢᵢE_{iib}E₄ | AlkL | E₄*E₅ | Eₐ |
| 747 | E₁ₐE₂E₃EᵢE₄ | AlkL | E₄*E₅ | Eₐ |
| 748 | E_{1b}E₂E₃EᵢE₄ | AlkL | E₄*E₅ | Eₐ |
| 749 | E₁ₐE₂E₃EᵢᵢE₄ | AlkL | E₄*E₅ | Eₐ |
| 750 | E_{1b}E₂E₃EᵢᵢE₄ | AlkL | E₄*E₅ | Eₐ |
| 751 | E₁ₐE₂E₃EᵢᵢE_{iib}E₄ | AlkL | E₄*E₅ | Eₐ |
| 752 | E_{1b}E₂E₃EᵢᵢE_{iib}E₄ | AlkL | E₄*E₅ | Eₐ |
| 753 | E₁ₐE₂Eᵢᵢᵢ | AlkL | E₄*E₅ | Eₐ |
| 754 | E_{1b}E₂Eᵢᵢᵢ | AlkL | E₄*E₅ | Eₐ |
| 755 | E₁ₐE₂E₃Eᵢᵢᵢ | AlkL | E₄*E₅ | Eₐ |
| 756 | E_{1b}E₂E₃Eᵢᵢᵢ | AlkL | E₄*E₅ | Eₐ |
| 757 | E₁ₐE₂EᵢᵢᵢE₄ | AlkL | E₄*E₅ | Eₐ |
| 758 | E_{1b}E₂EᵢᵢᵢE₄ | AlkL | E₄*E₅ | Eₐ |
| 759 | E₁ₐE₂E₃EᵢᵢᵢE₄ | AlkL | E₄*E₅ | Eₐ |
| 760 | E_{1b}E₂E₃EᵢᵢᵢE₄ | AlkL | E₄*E₅ | Eₐ |
| 761 | E₁ₐE₂Eᵢᵥ | AlkL | E₄*E₅ | Eₐ |
| 762 | E_{1b}E₂Eᵢᵥ | AlkL | E₄*E₅ | Eₐ |
| 763 | E₁ₐE₂E₃Eᵢᵥ | AlkL | E₄*E₅ | Eₐ |
| 764 | E_{1b}E₂E₃Eᵢᵥ | AlkL | E₄*E₅ | Eₐ |
| 765 | E₁ₐE₂EᵢᵥE₄ | AlkL | E₄*E₅ | Eₐ |
| 766 | E_{1b}E₂EᵢᵥE₄ | AlkL | E₄*E₅ | Eₐ |
| 767 | E₁ₐE₂E₃EᵢᵥE₄ | AlkL | E₄*E₅ | Eₐ |
| 768 | E_{1b}E₂E₃EᵢᵥE₄ | AlkL | E₄*E₅ | Eₐ |
| 769 | E₁ₐE₂ | | | E_{b} |
| 770 | E_{1b}E₂ | | | E_{b} |
| 771 | E₁ₐE₂E₃ | | | E_{b} |
| 772 | E_{1b}E₂E₃ | | | E_{b} |
| 773 | E₁ₐE₂Eᵢ | | | E_{b} |
| 774 | E_{1b}E₂Eᵢ | | | E_{b} |
| 775 | E₁ₐE₂Eᵢᵢ | | | E_{b} |
| 776 | E_{1b}E₂Eᵢᵢ | | | E_{b} |
| 777 | E₁ₐE₂EᵢᵢE_{iib} | | | E_{b} |
| 778 | E_{1b}E₂EᵢᵢE_{iib} | | | E_{b} |
| 779 | E₁ₐE₂E₃Eᵢ | | | E_{b} |
| 780 | E_{1b}E₂E₃Eᵢ | | | E_{b} |
| 781 | E₁ₐE₂E₃Eᵢᵢ | | | E_{b} |
| 782 | E_{1b}E₂E₃Eᵢᵢ | | | E_{b} |
| 783 | E₁ₐE₂E₃EᵢᵢE_{iib} | | | E_{b} |
| 784 | E_{1b}E₂E₃EᵢᵢE_{iib} | | | E_{b} |
| 785 | E₁ₐE₂E₄ | | | E_{b} |
| 786 | E_{1b}E₂E₄ | | | E_{b} |
| 787 | E₁ₐE₂E₃E₄ | | | E_{b} |
| 788 | E_{1b}E₂E₃E₄ | | | E_{b} |
| 789 | E₁ₐE₂EᵢE₄ | | | E_{b} |
| 790 | E_{1b}E₂EᵢE₄ | | | E_{b} |
| 791 | E₁ₐE₂EᵢᵢE₄ | | | E_{b} |
| 792 | E_{1b}E₂EᵢᵢE₄ | | | E_{b} |
| 793 | E₁ₐE₂EᵢᵢE_{iib}E₄ | | | E_{b} |
| 794 | E_{1b}E₂EᵢᵢE_{iib}E₄ | | | E_{b} |
| 795 | E₁ₐE₂E₃EᵢE₄ | | | E_{b} |
| 796 | E_{1b}E₂E₃EᵢE₄ | | | E_{b} |
| 797 | E₁ₐE₂E₃EᵢᵢE₄ | | | E_{b} |
| 798 | E_{1b}E₂E₃EᵢᵢE₄ | | | E_{b} |
| 799 | E₁ₐE₂E₃EᵢᵢE_{iib}E₄ | | | E_{b} |
| 800 | E_{1b}E₂E₃EᵢᵢE_{iib}E₄ | | | E_{b} |
| 801 | E₁ₐE₂Eᵢᵢᵢ | | | E_{b} |
| 802 | E_{1b}E₂Eᵢᵢᵢ | | | E_{b} |
| 803 | E₁ₐE₂E₃Eᵢᵢᵢ | | | E_{b} |
| 804 | E_{1b}E₂E₃Eᵢᵢᵢ | | | E_{b} |
| 805 | E₁ₐE₂EᵢᵢᵢE₄ | | | E_{b} |
| 806 | E_{1b}E₂EᵢᵢᵢE₄ | | | E_{b} |
| 807 | E₁ₐE₂E₃EᵢᵢᵢE₄ | | | E_{b} |
| 808 | E_{1b}E₂E₃EᵢᵢᵢE₄ | | | E_{b} |
| 809 | E₁ₐE₂Eᵢᵥ | | | E_{b} |
| 810 | E_{1b}E₂Eᵢᵥ | | | E_{b} |
| 811 | E₁ₐE₂E₃Eᵢᵥ | | | E_{b} |
| 812 | E_{1b}E₂E₃Eᵢᵥ | | | E_{b} |
| 813 | E₁ₐE₂EᵢᵥE₄ | | | E_{b} |
| 814 | E_{1b}E₂EᵢᵥE₄ | | | E_{b} |
| 815 | E₁ₐE₂E₃EᵢᵥE₄ | | | E_{b} |
| 816 | E_{1b}E₂E₃EᵢᵥE₄ | | | E_{b} |
| 817 | E₁ₐE₂ | | E₄* | E_{b} |
| 818 | E_{1b}E₂ | | E₄* | E_{b} |
| 819 | E₁ₐE₂E₃ | | E₄* | E_{b} |
| 820 | E_{1b}E₂E₃ | | E₄* | E_{b} |
| 821 | E₁ₐE₂Eᵢ | | E₄* | E_{b} |
| 822 | E_{1b}E₂Eᵢ | | E₄* | E_{b} |
| 823 | E₁ₐE₂Eᵢᵢ | | E₄* | E_{b} |
| 824 | E_{1b}E₂Eᵢᵢ | | E₄* | E_{b} |
| 825 | E₁ₐE₂EᵢᵢE_{iib} | | E₄* | E_{b} |
| 826 | E_{1b}E₂EᵢᵢE_{iib} | | E₄* | E_{b} |
| 827 | E₁ₐE₂E₃Eᵢ | | E₄* | E_{b} |
| 828 | E_{1b}E₂E₃Eᵢ | | E₄* | E_{b} |
| 829 | E₁ₐE₂E₃Eᵢᵢ | | E₄* | E_{b} |
| 830 | E_{1b}E₂E₃Eᵢᵢ | | E₄* | E_{b} |
| 831 | E₁ₐE₂E₃EᵢᵢE_{iib} | | E₄* | E_{b} |
| 832 | E_{1b}E₂E₃EᵢᵢE_{iib} | | E₄* | E_{b} |
| 833 | E₁ₐE₂E₄ | | E₄* | E_{b} |
| 834 | E_{1b}E₂E₄ | | E₄* | E_{b} |
| 835 | E₁ₐE₂E₃E₄ | | E₄* | E_{b} |
| 836 | E_{1b}E₂E₃E₄ | | E₄* | E_{b} |
| 837 | E₁ₐE₂EᵢE₄ | | E₄* | E_{b} |
| 838 | E_{1b}E₂EᵢE₄ | | E₄* | E_{b} |
| 839 | E₁ₐE₂EᵢᵢE₄ | | E₄* | E_{b} |
| 840 | E_{1b}E₂EᵢᵢE₄ | | E₄* | E_{b} |
| 841 | E₁ₐE₂EᵢᵢE_{iib}E₄ | | E₄* | E_{b} |
| 842 | E_{1b}E₂EᵢᵢE_{iib}E₄ | | E₄* | E_{b} |
| 843 | E₁ₐE₂E₃EᵢE₄ | | E₄* | E_{b} |
| 844 | E_{1b}E₂E₃EᵢE₄ | | E₄* | E_{b} |
| 845 | E₁ₐE₂E₃EᵢᵢE₄ | | E₄* | E_{b} |
| 846 | E_{1b}E₂E₃EᵢᵢE₄ | | E₄* | E_{b} |
| 847 | E₁ₐE₂E₃EᵢᵢE_{iib}E₄ | | E₄* | E_{b} |
| 848 | E_{1b}E₂E₃EᵢᵢE_{iib}E₄ | | E₄* | E_{b} |
| 849 | E₁ₐE₂Eᵢᵢᵢ | | E₄* | E_{b} |
| 850 | E_{1b}E₂Eᵢᵢᵢ | | E₄* | E_{b} |
| 851 | E₁ₐE₂E₃Eᵢᵢᵢ | | E₄* | E_{b} |
| 852 | E_{1b}E₂E₃Eᵢᵢᵢ | | E₄* | E_{b} |
| 853 | E₁ₐE₂EᵢᵢᵢE₄ | | E₄* | E_{b} |
| 854 | E_{1b}E₂EᵢᵢᵢE₄ | | E₄* | E_{b} |
| 855 | E₁ₐE₂E₃EᵢᵢᵢE₄ | | E₄* | E_{b} |
| 856 | E_{1b}E₂E₃EᵢᵢᵢE₄ | | E₄* | E_{b} |
| 857 | E₁ₐE₂Eᵢᵥ | | E₄* | E_{b} |
| 858 | E_{1b}E₂Eᵢᵥ | | E₄* | E_{b} |
| 859 | E₁ₐE₂E₃Eᵢᵥ | | E₄* | E_{b} |
| 860 | E_{1b}E₂E₃Eᵢᵥ | | E₄* | E_{b} |
| 861 | E₁ₐE₂EᵢᵥE₄ | | E₄* | E_{b} |
| 862 | E_{1b}E₂EᵢᵥE₄ | | E₄* | E_{b} |
| 863 | E₁ₐE₂E₃EᵢᵥE₄ | | E₄* | E_{b} |
| 864 | E_{1b}E₂E₃EᵢᵥE₄ | | E₄* | E_{b} |
| 865 | E₁ₐE₂ | | E₅ | E_{b} |
| 866 | E_{1b}E₂ | | E₅ | E_{b} |
| 867 | E₁ₐE₂E₃ | | E₅ | E_{b} |
| 868 | E_{1b}E₂E₃ | | E₅ | E_{b} |
| 869 | E₁ₐE₂Eᵢ | | E₅ | E_{b} |
| 870 | E_{1b}E₂Eᵢ | | E₅ | E_{b} |
| 871 | E₁ₐE₂Eᵢᵢ | | E₅ | E_{b} |
| 872 | E_{1b}E₂Eᵢᵢ | | E₅ | E_{b} |
| 873 | E₁ₐE₂EᵢᵢE_{iib} | | E₅ | E_{b} |
| 874 | E_{1b}E₂EᵢᵢE_{iib} | | E₅ | E_{b} |
| 875 | E₁ₐE₂E₃Eᵢ | | E₅ | E_{b} |
| 876 | E_{1b}E₂E₃Eᵢ | | E₅ | E_{b} |
| 877 | E₁ₐE₂E₃Eᵢᵢ | | E₅ | E_{b} |
| 878 | E_{1b}E₂E₃Eᵢᵢ | | E₅ | E_{b} |
| 879 | E₁ₐE₂E₃EᵢᵢE_{iib} | | E₅ | E_{b} |
| 880 | E_{1b}E₂E₃EᵢᵢE_{iib} | | E₅ | E_{b} |
| 881 | E₁ₐE₂E₄ | | E₅ | E_{b} |
| 882 | E_{1b}E₂E₄ | | E₅ | E_{b} |
| 883 | E₁ₐE₂E₃E₄ | | E₅ | E_{b} |
| 884 | E_{1b}E₂E₃E₄ | | E₅ | E_{b} |
| 885 | E₁ₐE₂EᵢE₄ | | E₅ | E_{b} |
| 886 | E_{1b}E₂EᵢE₄ | | E₅ | E_{b} |
| 887 | E₁ₐE₂EᵢᵢE₄ | | E₅ | E_{b} |
| 888 | E_{1b}E₂EᵢᵢE₄ | | E₅ | E_{b} |
| 889 | E₁ₐE₂EᵢᵢE_{iib}E₄ | | E₅ | E_{b} |
| 890 | E_{1b}E₂EᵢᵢE_{iib}E₄ | | E₅ | E_{b} |
| 891 | E₁ₐE₂E₃EᵢE₄ | | E₅ | E_{b} |
| 892 | E_{1b}E₂E₃EᵢE₄ | | E₅ | E_{b} |
| 893 | E₁ₐE₂E₃EᵢᵢE₄ | | E₅ | E_{b} |
| 894 | E_{1b}E₂E₃EᵢᵢE₄ | | E₅ | E_{b} |
| 895 | E₁ₐE₂E₃EᵢᵢE_{iib}E₄ | | E₅ | E_{b} |
| 896 | E_{1b}E₂E₃EᵢᵢE_{iib}E₄ | | E₅ | E_{b} |
| 897 | E₁ₐE₂Eᵢᵢᵢ | | E₅ | E_{b} |
| 898 | E_{1b}E₂Eᵢᵢᵢ | | E₅ | E_{b} |
| 899 | E₁ₐE₂E₃Eᵢᵢᵢ | | E₅ | E_{b} |
| 900 | E_{1b}E₂E₃Eᵢᵢᵢ | | E₅ | E_{b} |
| 901 | E₁ₐE₂EᵢᵢᵢE₄ | | E₅ | E_{b} |
| 902 | E_{1b}E₂EᵢᵢᵢE₄ | | E₅ | E_{b} |
| 903 | E₁ₐE₂E₃EᵢᵢᵢE₄ | | E₅ | E_{b} |
| 904 | E_{1b}E₂E₃EᵢᵢᵢE₄ | | E₅ | E_{b} |
| 905 | E₁ₐE₂Eᵢᵥ | | E₅ | E_{b} |
| 906 | E_{1b}E₂Eᵢᵥ | | E₅ | E_{b} |
| 907 | E₁ₐE₂E₃Eᵢᵥ | | E₅ | E_{b} |
| 908 | E_{1b}E₂E₃Eᵢᵥ | | E₅ | E_{b} |
| 909 | E₁ₐE₂EᵢᵥE₄ | | E₅ | E_{b} |
| 910 | E_{1b}E₂EᵢᵥE₄ | | E₅ | E_{b} |
| 911 | E₁ₐE₂E₃EᵢᵥE₄ | | E₅ | E_{b} |
| 912 | E_{1b}E₂E₃EᵢᵥE₄ | | E₅ | E_{b} |
| 913 | E₁ₐE₂ | | E₄*E₅ | E_{b} |
| 914 | E_{1b}E₂ | | E₄*E₅ | E_{b} |
| 915 | E₁ₐE₂E₃ | | E₄*E₅ | E_{b} |
| 916 | E_{1b}E₂E₃ | | E₄*E₅ | E_{b} |
| 917 | E₁ₐE₂Eᵢ | | E₄*E₅ | E_{b} |
| 918 | E_{1b}E₂Eᵢ | | E₄*E₅ | E_{b} |
| 919 | E₁ₐE₂Eᵢᵢ | | E₄*E₅ | E_{b} |
| 920 | E_{1b}E₂Eᵢᵢ | | E₄*E₅ | E_{b} |
| 921 | E₁ₐE₂EᵢᵢE_{iib} | | E₄*E₅ | E_{b} |
| 922 | E_{1b}E₂EᵢᵢE_{iib} | | E₄*E₅ | E_{b} |
| 923 | E₁ₐE₂E₃Eᵢ | | E₄*E₅ | E_{b} |
| 924 | E_{1b}E₂E₃Eᵢ | | E₄*E₅ | E_{b} |
| 925 | E₁ₐE₂E₃Eᵢᵢ | | E₄*E₅ | E_{b} |
| 926 | E_{1b}E₂E₃Eᵢᵢ | | E₄*E₅ | E_{b} |
| 927 | E₁ₐE₂E₃EᵢᵢE_{iib} | | E₄*E₅ | E_{b} |
| 928 | E_{1b}E₂E₃EᵢᵢE_{iib} | | E₄*E₅ | E_{b} |
| 929 | E₁ₐE₂E₄ | | E₄*E₅ | E_{b} |
| 930 | E_{1b}E₂E₄ | | E₄*E₅ | E_{b} |
| 931 | E₁ₐE₂E₃E₄ | | E₄*E₅ | E_{b} |
| 932 | E_{1b}E₂E₃E₄ | | E₄*E₅ | E_{b} |
| 933 | E₁ₐE₂EᵢE₄ | | E₄*E₅ | E_{b} |
| 934 | E_{1b}E₂EᵢE₄ | | E₄*E₅ | E_{b} |
| 935 | E₁ₐE₂EᵢᵢE₄ | | E₄*E₅ | E_{b} |
| 936 | E_{1b}E₂EᵢᵢE₄ | | E₄*E₅ | E_{b} |
| 937 | E₁ₐE₂EᵢᵢE_{iib}E₄ | | E₄*E₅ | E_{b} |
| 938 | E_{1b}E₂EᵢᵢE_{iib}E₄ | | E₄*E₅ | E_{b} |
| 939 | E₁ₐE₂E₃EᵢE₄ | | E₄*E₅ | E_{b} |
| 940 | E_{1b}E₂E₃EᵢE₄ | | E₄*E₅ | E_{b} |
| 941 | E₁ₐE₂E₃EᵢᵢE₄ | | E₄*E₅ | E_{b} |
| 942 | E_{1b}E₂E₃EᵢᵢE₄ | | E₄*E₅ | E_{b} |
| 943 | E₁ₐE₂E₃EᵢᵢE_{iib}E₄ | | E₄*E₅ | E_{b} |
| 944 | E_{1b}E₂E₃EᵢᵢE_{iib}E₄ | | E₄*E₅ | E_{b} |
| 945 | E₁ₐE₂Eᵢᵢᵢ | | E₄*E₅ | E_{b} |
| 946 | E_{1b}E₂Eᵢᵢᵢ | | E₄*E₅ | E_{b} |
| 947 | E₁ₐE₂E₃Eᵢᵢᵢ | | E₄*E₅ | E_{b} |
| 948 | E_{1b}E₂E₃Eᵢᵢᵢ | | E₄*E₅ | E_{b} |
| 949 | E₁ₐE₂EᵢᵢᵢE₄ | | E₄*E₅ | E_{b} |
| 950 | E_{1b}E₂EᵢᵢᵢE₄ | | E₄*E₅ | E_{b} |
| 951 | E₁ₐE₂E₃EᵢᵢᵢE₄ | | E₄*E₅ | E_{b} |
| 952 | E_{1b}E₂E₃EᵢᵢᵢE₄ | | E₄*E₅ | E_{b} |
| 953 | E₁ₐE₂Eᵢᵥ | | E₄*E₅ | E_{b} |
| 954 | E_{1b}E₂Eᵢᵥ | | E₄*E₅ | E_{b} |
| 955 | E₁ₐE₂E₃Eᵢᵥ | | E₄*E₅ | E_{b} |
| 956 | E_{1b}E₂E₃Eᵢᵥ | | E₄*E₅ | E_{b} |
| 957 | E₁ₐE₂EᵢᵥE₄ | | E₄*E₅ | E_{b} |
| 958 | E_{1b}E₂EᵢᵥE₄ | | E₄*E₅ | E_{b} |
| 959 | E₁ₐE₂E₃EᵢᵥE₄ | | E₄*E₅ | E_{b} |
| 960 | E_{1b}E₂E₃EᵢᵥE₄ | | E₄*E₅ | E_{b} |
| 961 | E₁ₐE₂ | AlkL | | E_{b} |
| 962 | E_{1b}E₂ | AlkL | | E_{b} |
| 963 | E₁ₐE₂E₃ | AlkL | | E_{b} |
| 964 | E_{1b}E₂E₃ | AlkL | | E_{b} |
| 965 | E₁ₐE₂Eᵢ | AlkL | | E_{b} |
| 966 | E_{1b}E₂Eᵢ | AlkL | | E_{b} |
| 967 | E₁ₐE₂Eᵢᵢ | AlkL | | E_{b} |
| 968 | E_{1b}E₂Eᵢᵢ | AlkL | | E_{b} |
| 969 | E₁ₐE₂EᵢᵢE_{iib} | AlkL | | E_{b} |
| 970 | E_{1b}E₂EᵢᵢE_{iib} | AlkL | | E_{b} |
| 971 | E₁ₐE₂E₃Eᵢ | AlkL | | E_{b} |
| 972 | E_{1b}E₂E₃Eᵢ | AlkL | | E_{b} |
| 973 | E₁ₐE₂E₃Eᵢᵢ | AlkL | | E_{b} |
| 974 | E_{1b}E₂E₃Eᵢᵢ | AlkL | | E_{b} |
| 975 | E₁ₐE₂E₃EᵢᵢE_{iib} | AlkL | | E_{b} |
| 976 | E_{1b}E₂E₃EᵢᵢE_{iib} | AlkL | | E_{b} |
| 977 | E₁ₐE₂E₄ | AlkL | | E_{b} |
| 978 | E_{1b}E₂E₄ | AlkL | | E_{b} |
| 979 | E₁ₐE₂E₃E₄ | AlkL | | E_{b} |
| 980 | E_{1b}E₂E₃E₄ | AlkL | | E_{b} |
| 981 | E₁ₐE₂EᵢE₄ | AlkL | | E_{b} |
| 982 | E_{1b}E₂EᵢE₄ | AlkL | | E_{b} |
| 983 | E₁ₐE₂EᵢᵢE₄ | AlkL | | E_{b} |
| 984 | E_{1b}E₂EᵢᵢE₄ | AlkL | | E_{b} |
| 985 | E₁ₐE₂EᵢᵢE_{iib}E₄ | AlkL | | E_{b} |
| 986 | E_{1b}E₂EᵢᵢE_{iib}E₄ | AlkL | | E_{b} |
| 987 | E₁ₐE₂E₃EᵢE₄ | AlkL | | E_{b} |
| 988 | E_{1b}E₂E₃EᵢE₄ | AlkL | | E_{b} |
| 989 | E₁ₐE₂E₃EᵢᵢE₄ | AlkL | | E_{b} |
| 990 | E_{1b}E₂E₃EᵢᵢE₄ | AlkL | | E_{b} |
| 991 | E₁ₐE₂E₃EᵢᵢE_{iib}E₄ | AlkL | | E_{b} |
| 992 | E_{1b}E₂E₃EᵢᵢE_{iib}E₄ | AlkL | | E_{b} |
| 993 | E₁ₐE₂Eᵢᵢᵢ | AlkL | | E_{b} |
| 994 | E_{1b}E₂Eᵢᵢᵢ | AlkL | | E_{b} |
| 995 | E₁ₐE₂E₃Eᵢᵢᵢ | AlkL | | E_{b} |
| 996 | E_{1b}E₂E₃Eᵢᵢᵢ | AlkL | | E_{b} |
| 997 | E₁ₐE₂EᵢᵢᵢE₄ | AlkL | | E_{b} |
| 998 | E_{1b}E₂EᵢᵢᵢE₄ | AlkL | | E_{b} |
| 999 | E₁ₐE₂E₃EᵢᵢᵢE₄ | AlkL | | E_{b} |
| 1000 | E_{1b}E₂E₃EᵢᵢᵢE₄ | AlkL | | E_{b} |
| 1001 | E₁ₐE₂Eᵢᵥ | AlkL | | E_{b} |
| 1002 | E_{1b}E₂Eᵢᵥ | AlkL | | E_{b} |
| 1003 | E₁ₐE₂E₃Eᵢᵥ | AlkL | | E_{b} |
| 1004 | E_{1b}E₂E₃Eᵢᵥ | AlkL | | E_{b} |
| 1005 | E₁ₐE₂EᵢᵥE₄ | AlkL | | E_{b} |
| 1006 | E_{1b}E₂EᵢᵥE₄ | AlkL | | E_{b} |
| 1007 | E₁ₐE₂E₃EᵢᵥE₄ | AlkL | | E_{b} |
| 1008 | E_{1b}E₂E₃EᵢᵥE₄ | AlkL | | E_{b} |
| 1009 | E₁ₐE₂ | AlkL | E₄* | E_{b} |
| 1010 | E_{1b}E₂ | AlkL | E₄* | E_{b} |
| 1011 | E₁ₐE₂E₃ | AlkL | E₄* | E_{b} |
| 1012 | E_{1b}E₂E₃ | AlkL | E₄* | E_{b} |
| 1013 | E₁ₐE₂Eᵢ | AlkL | E₄* | E_{b} |
| 1014 | E_{1b}E₂Eᵢ | AlkL | E₄* | E_{b} |
| 1015 | E₁ₐE₂Eᵢᵢ | AlkL | E₄* | E_{b} |
| 1016 | E_{1b}E₂Eᵢᵢ | AlkL | E₄* | E_{b} |
| 1017 | E₁ₐE₂EᵢᵢE_{iib} | AlkL | E₄* | E_{b} |
| 1018 | E_{1b}E₂EᵢᵢE_{iib} | AlkL | E₄* | E_{b} |
| 1019 | E₁ₐE₂E₃Eᵢ | AlkL | E₄* | E_{b} |
| 1020 | E_{1b}E₂E₃Eᵢ | AlkL | E₄* | E_{b} |
| 1021 | E₁ₐE₂E₃Eᵢᵢ | AlkL | E₄* | E_{b} |
| 1022 | E_{1b}E₂E₃Eᵢᵢ | AlkL | E₄* | E_{b} |
| 1023 | E₁ₐE₂E₃EᵢᵢE_{iib} | AlkL | E₄* | E_{b} |
| 1024 | E_{1b}E₂E₃EᵢᵢE_{iib} | AlkL | E₄* | E_{b} |
| 1025 | E₁ₐE₂E₄ | AlkL | E₄* | E_{b} |
| 1026 | E_{1b}E₂E₄ | AlkL | E₄* | E_{b} |
| 1027 | E₁ₐE₂E₃E₄ | AlkL | E₄* | E_{b} |
| 1028 | E_{1b}E₂E₃E₄ | AlkL | E₄* | E_{b} |
| 1029 | E₁ₐE₂EᵢE₄ | AlkL | E₄* | E_{b} |
| 1030 | E_{1b}E₂EᵢE₄ | AlkL | E₄* | E_{b} |
| 1031 | E₁ₐE₂EᵢᵢE₄ | AlkL | E₄* | E_{b} |
| 1032 | E_{1b}E₂EᵢᵢE₄ | AlkL | E₄* | E_{b} |
| 1033 | E₁ₐE₂EᵢᵢE_{iib}E₄ | AlkL | E₄* | E_{b} |
| 1034 | E_{1b}E₂EᵢᵢE_{iib}E₄ | AlkL | E₄* | E_{b} |
| 1035 | E₁ₐE₂E₃EᵢE₄ | AlkL | E₄* | E_{b} |
| 1036 | E_{1b}E₂E₃EᵢE₄ | AlkL | E₄* | E_{b} |
| 1037 | E₁ₐE₂E₃EᵢᵢE₄ | AlkL | E₄* | E_{b} |
| 1038 | E_{1b}E₂E₃EᵢᵢE₄ | AlkL | E₄* | E_{b} |
| 1039 | E₁ₐE₂E₃EᵢᵢE_{iib}E₄ | AlkL | E₄* | E_{b} |
| 1040 | E_{1b}E₂E₃EᵢᵢE_{iib}E₄ | AlkL | E₄* | E_{b} |
| 1041 | E₁ₐE₂Eᵢᵢᵢ | AlkL | E₄* | E_{b} |
| 1042 | E_{1b}E₂Eᵢᵢᵢ | AlkL | E₄* | E_{b} |
| 1043 | E₁ₐE₂E₃Eᵢᵢᵢ | AlkL | E₄* | E_{b} |
| 1044 | E_{1b}E₂E₃Eᵢᵢᵢ | AlkL | E₄* | E_{b} |
| 1045 | E₁ₐE₂EᵢᵢᵢE₄ | AlkL | E₄* | E_{b} |
| 1046 | E_{1b}E₂EᵢᵢᵢE₄ | AlkL | E₄* | E_{b} |
| 1047 | E₁ₐE₂E₃EᵢᵢᵢE₄ | AlkL | E₄* | E_{b} |
| 1048 | E_{1b}E₂E₃EᵢᵢᵢE₄ | AlkL | E₄* | E_{b} |
| 1049 | E₁ₐE₂Eᵢᵥ | AlkL | E₄* | E_{b} |
| 1050 | E_{1b}E₂Eᵢᵥ | AlkL | E₄* | E_{b} |
| 1051 | E₁ₐE₂E₃Eᵢᵥ | AlkL | E₄* | E_{b} |
| 1052 | E_{1b}E₂E₃Eᵢᵥ | AlkL | E₄* | E_{b} |
| 1053 | E₁ₐE₂EᵢᵥE₄ | AlkL | E₄* | E_{b} |
| 1054 | E_{1b}E₂EᵢᵥE₄ | AlkL | E₄* | E_{b} |
| 1055 | E₁ₐE₂E₃EᵢᵥE₄ | AlkL | E₄* | E_{b} |
| 1056 | E_{1b}E₂E₃EᵢᵥE₄ | AlkL | E₄* | E_{b} |
| 1057 | E₁ₐE₂ | AlkL | E₅ | E_{b} |
| 1058 | E_{1b}E₂ | AlkL | E₅ | E_{b} |
| 1059 | E₁ₐE₂E₃ | AlkL | E₅ | E_{b} |
| 1060 | E_{1b}E₂E₃ | AlkL | E₅ | E_{b} |
| 1061 | E₁ₐE₂Eᵢ | AlkL | E₅ | E_{b} |
| 1062 | E_{1b}E₂Eᵢ | AlkL | E₅ | E_{b} |
| 1063 | E₁ₐE₂Eᵢᵢ | AlkL | E₅ | E_{b} |
| 1064 | E_{1b}E₂Eᵢᵢ | AlkL | E₅ | E_{b} |
| 1065 | E₁ₐE₂EᵢᵢE_{iib} | AlkL | E₅ | E_{b} |
| 1066 | E_{1b}E₂EᵢᵢE_{iib} | AlkL | E₅ | E_{b} |
| 1067 | E₁ₐE₂E₃Eᵢ | AlkL | E₅ | E_{b} |
| 1068 | E_{1b}E₂E₃Eᵢ | AlkL | E₅ | E_{b} |
| 1069 | E₁ₐE₂E₃Eᵢᵢ | AlkL | E₅ | E_{b} |
| 1070 | E_{1b}E₂E₃Eᵢᵢ | AlkL | E₅ | E_{b} |
| 1071 | E₁ₐE₂E₃EᵢᵢE_{iib} | AlkL | E₅ | E_{b} |
| 1072 | E_{1b}E₂E₃EᵢᵢE_{iib} | AlkL | E₅ | E_{b} |
| 1073 | E₁ₐE₂E₄ | AlkL | E₅ | E_{b} |
| 1074 | E_{1b}E₂E₄ | AlkL | E₅ | E_{b} |
| 1075 | E₁ₐE₂E₃E₄ | AlkL | E₅ | E_{b} |
| 1076 | E_{1b}E₂E₃E₄ | AlkL | E₅ | E_{b} |
| 1077 | E₁ₐE₂EᵢE₄ | AlkL | E₅ | E_{b} |
| 1078 | E_{1b}E₂EᵢE₄ | AlkL | E₅ | E_{b} |
| 1079 | E₁ₐE₂EᵢᵢE₄ | AlkL | E₅ | E_{b} |
| 1080 | E_{1b}E₂EᵢᵢE₄ | AlkL | E₅ | E_{b} |
| 1081 | E₁ₐE₂EᵢᵢE_{iib}E₄ | AlkL | E₅ | E_{b} |
| 1082 | E_{1b}E₂EᵢᵢE_{iib}E₄ | AlkL | E₅ | E_{b} |
| 1083 | E₁ₐE₂E₃EᵢE₄ | AlkL | E₅ | E_{b} |
| 1084 | E_{1b}E₂E₃EᵢE₄ | AlkL | E₅ | E_{b} |
| 1085 | E₁ₐE₂E₃EᵢᵢE₄ | AlkL | E₅ | E_{b} |
| 1086 | E_{1b}E₂E₃EᵢᵢE₄ | AlkL | E₅ | E_{b} |
| 1087 | E₁ₐE₂E₃EᵢᵢE_{iib}E₄ | AlkL | E₅ | E_{b} |
| 1088 | E_{1b}E₂E₃EᵢᵢE_{iib}E₄ | AlkL | E₅ | E_{b} |
| 1089 | E₁ₐE₂Eᵢᵢᵢ | AlkL | E₅ | E_{b} |
| 1090 | E_{1b}E₂Eᵢᵢᵢ | AlkL | E₅ | E_{b} |
| 1091 | E₁ₐE₂E₃Eᵢᵢᵢ | AlkL | E₅ | E_{b} |
| 1092 | E_{1b}E₂E₃Eᵢᵢᵢ | AlkL | E₅ | E_{b} |
| 1093 | E₁ₐE₂EᵢᵢᵢE₄ | AlkL | E₅ | E_{b} |
| 1094 | E_{1b}E₂EᵢᵢᵢE₄ | AlkL | E₅ | E_{b} |
| 1095 | E₁ₐE₂E₃EᵢᵢᵢE₄ | AlkL | E₅ | E_{b} |
| 1096 | E_{1b}E₂E₃EᵢᵢᵢE₄ | AlkL | E₅ | E_{b} |
| 1097 | E₁ₐE₂Eᵢᵥ | AlkL | E₅ | E_{b} |
| 1098 | E_{1b}E₂Eᵢᵥ | AlkL | E₅ | E_{b} |
| 1099 | E₁ₐE₂E₃Eᵢᵥ | AlkL | E₅ | E_{b} |
| 1100 | E_{1b}E₂E₃Eᵢᵥ | AlkL | E₅ | E_{b} |
| 1101 | E₁ₐE₂EᵢᵥE₄ | AlkL | E₅ | E_{b} |
| 1102 | E_{1b}E₂EᵢᵥE₄ | AlkL | E₅ | E_{b} |
| 1103 | E₁ₐE₂E₃EᵢᵥE₄ | AlkL | E₅ | E_{b} |
| 1104 | E_{1b}E₂E₃EᵢᵥE₄ | AlkL | E₅ | E_{b} |
| 1105 | E₁ₐE₂ | AlkL | E₄*E₅ | E_{b} |
| 1106 | E_{1b}E₂ | AlkL | E₄*E₅ | E_{b} |
| 1107 | E₁ₐE₂E₃ | AlkL | E₄*E₅ | E_{b} |
| 1108 | E_{1b}E₂E₃ | AlkL | E₄*E₅ | E_{b} |
| 1109 | E₁ₐE₂Eᵢ | AlkL | E₄*E₅ | E_{b} |
| 1110 | E_{1b}E₂Eᵢ | AlkL | E₄*E₅ | E_{b} |
| 1111 | E₁ₐE₂Eᵢᵢ | AlkL | E₄*E₅ | E_{b} |
| 1112 | E_{1b}E₂Eᵢᵢ | AlkL | E₄*E₅ | E_{b} |
| 1113 | E₁ₐE₂EᵢᵢE_{iib} | AlkL | E₄*E₅ | E_{b} |
| 1114 | E_{1b}E₂EᵢᵢE_{iib} | AlkL | E₄*E₅ | E_{b} |
| 1115 | E₁ₐE₂E₃Eᵢ | AlkL | E₄*E₅ | E_{b} |
| 1116 | E_{1b}E₂E₃Eᵢ | AlkL | E₄*E₅ | E_{b} |
| 1117 | E₁ₐE₂E₃Eᵢᵢ | AlkL | E₄*E₅ | E_{b} |
| 1118 | E_{1b}E₂E₃Eᵢᵢ | AlkL | E₄*E₅ | E_{b} |
| 1119 | E₁ₐE₂E₃EᵢᵢE_{iib} | AlkL | E₄*E₅ | E_{b} |
| 1120 | E_{1b}E₂E₃EᵢᵢE_{iib} | AlkL | E₄*E₅ | E_{b} |
| 1121 | E₁ₐE₂E₄ | AlkL | E₄*E₅ | E_{b} |
| 1122 | E_{1b}E₂E₄ | AlkL | E₄*E₅ | E_{b} |
| 1123 | E₁ₐE₂E₃E₄ | AlkL | E₄*E₅ | E_{b} |
| 1124 | E_{1b}E₂E₃E₄ | AlkL | E₄*E₅ | E_{b} |
| 1125 | E₁ₐE₂EᵢE₄ | AlkL | E₄*E₅ | E_{b} |
| 1126 | E_{1b}E₂EᵢE₄ | AlkL | E₄*E₅ | E_{b} |
| 1127 | E₁ₐE₂EᵢᵢE₄ | AlkL | E₄*E₅ | E_{b} |
| 1128 | E_{1b}E₂EᵢᵢE₄ | AlkL | E₄*E₅ | E_{b} |
| 1129 | E₁ₐE₂EᵢᵢE_{iib}E₄ | AlkL | E₄*E₅ | E_{b} |
| 1130 | E_{1b}E₂EᵢᵢE_{iib}E₄ | AlkL | E₄*E₅ | E_{b} |
| 1131 | E₁ₐE₂E₃EᵢE₄ | AlkL | E₄*E₅ | E_{b} |
| 1132 | E_{1b}E₂E₃EᵢE₄ | AlkL | E₄*E₅ | E_{b} |
| 1133 | E₁ₐE₂E₃EᵢᵢE₄ | AlkL | E₄*E₅ | E_{b} |
| 1134 | E_{1b}E₂E₃EᵢᵢE₄ | AlkL | E₄*E₅ | E_{b} |
| 1135 | E₁ₐE₂E₃EᵢᵢE_{iib}E₄ | AlkL | E₄*E₅ | E_{b} |
| 1136 | E_{1b}E₂E₃EᵢᵢE_{iib}E₄ | AlkL | E₄*E₅ | E_{b} |
| 1137 | E₁ₐE₂Eᵢᵢᵢ | AlkL | E₄*E₅ | E_{b} |
| 1138 | E_{1b}E₂Eᵢᵢᵢ | AlkL | E₄*E₅ | E_{b} |
| 1139 | E₁ₐE₂E₃Eᵢᵢᵢ | AlkL | E₄*E₅ | E_{b} |
| 1140 | E_{1b}E₂E₃Eᵢᵢᵢ | AlkL | E₄*E₅ | E_{b} |
| 1141 | E₁ₐE₂EᵢᵢᵢE₄ | AlkL | E₄*E₅ | E_{b} |
| 1142 | E_{1b}E₂EᵢᵢᵢE₄ | AlkL | E₄*E₅ | E_{b} |
| 1143 | E₁ₐE₂E₃EᵢᵢᵢE₄ | AlkL | E₄*E₅ | E_{b} |
| 1144 | E_{1b}E₂E₃EᵢᵢᵢE₄ | AlkL | E₄*E₅ | E_{b} |
| 1145 | E₁ₐE₂Eᵢᵥ | AlkL | E₄*E₅ | E_{b} |
| 1146 | E_{1b}E₂Eᵢᵥ | AlkL | E₄*E₅ | E_{b} |
| 1147 | E₁ₐE₂E₃Eᵢᵥ | AlkL | E₄*E₅ | E_{b} |
| 1148 | E_{1b}E₂E₃Eᵢᵥ | AlkL | E₄*E₅ | E_{b} |
| 1149 | E₁ₐE₂EᵢᵥE₄ | AlkL | E₄*E₅ | E_{b} |
| 1150 | E_{1b}E₂EᵢᵥE₄ | AlkL | E₄*E₅ | E_{b} |
| 1151 | E₁ₐE₂E₃EᵢᵥE₄ | AlkL | E₄*E₅ | E_{b} |
| 1152 | E_{1b}E₂E₃EᵢᵥE₄ | AlkL | E₄*E₅ | E_{b} |
| 1153 | E₁ₐE₂ | | | E_{d} |
| 1154 | E_{1b}E₂ | | | E_{d} |
| 1155 | E₁ₐE₂E₃ | | | E_{d} |
| 1156 | E_{1b}E₂E₃ | | | E_{d} |
| 1157 | E₁ₐE₂Eᵢ | | | E_{d} |
| 1158 | E_{1b}E₂Eᵢ | | | E_{d} |
| 1159 | E₁ₐE₂Eᵢᵢ | | | E_{d} |
| 1160 | E_{1b}E₂Eᵢᵢ | | | E_{d} |
| 1161 | E₁ₐE₂EᵢᵢE_{iib} | | | E_{d} |
| 1162 | E_{1b}E₂EᵢᵢE_{iib} | | | E_{d} |
| 1163 | E₁ₐE₂E₃Eᵢ | | | E_{d} |
| 1164 | E_{1b}E₂E₃Eᵢ | | | E_{d} |
| 1165 | E₁ₐE₂E₃Eᵢᵢ | | | E_{d} |
| 1166 | E_{1b}E₂E₃Eᵢᵢ | | | E_{d} |
| 1167 | E₁ₐE₂E₃EᵢᵢE_{iib} | | | E_{d} |
| 1168 | E_{1b}E₂E₃EᵢᵢE_{iib} | | | E_{d} |
| 1169 | E₁ₐE₂E₄ | | | E_{d} |
| 1170 | E_{1b}E₂E₄ | | | E_{d} |
| 1171 | E₁ₐE₂E₃E₄ | | | E_{d} |
| 1172 | E_{1b}E₂E₃E₄ | | | E_{d} |
| 1173 | E₁ₐE₂EᵢE₄ | | | E_{d} |
| 1174 | E_{1b}E₂EᵢE₄ | | | E_{d} |
| 1175 | E₁ₐE₂EᵢᵢE₄ | | | E_{d} |
| 1176 | E_{1b}E₂EᵢᵢE₄ | | | E_{d} |
| 1177 | E₁ₐE₂EᵢᵢE_{iib}E₄ | | | E_{d} |
| 1178 | E_{1b}E₂EᵢᵢE_{iib}E₄ | | | E_{d} |
| 1179 | E₁ₐE₂E₃EᵢE₄ | | | E_{d} |
| 1180 | E_{1b}E₂E₃EᵢE₄ | | | E_{d} |
| 1181 | E₁ₐE₂E₃EᵢᵢE₄ | | | E_{d} |
| 1182 | E_{1b}E₂E₃EᵢᵢE₄ | | | E_{d} |
| 1183 | E₁ₐE₂E₃EᵢᵢE_{iib}E₄ | | | E_{d} |
| 1184 | E_{1b}E₂E₃EᵢᵢE_{iib}E₄ | | | E_{d} |
| 1185 | E₁ₐE₂Eᵢᵢᵢ | | | E_{d} |
| 1186 | E_{1b}E₂Eᵢᵢᵢ | | | E_{d} |
| 1187 | E₁ₐE₂E₃Eᵢᵢᵢ | | | E_{d} |
| 1188 | E_{1b}E₂E₃Eᵢᵢᵢ | | | E_{d} |
| 1189 | E₁ₐE₂EᵢᵢᵢE₄ | | | E_{d} |
| 1190 | E_{1b}E₂EᵢᵢᵢE₄ | | | E_{d} |
| 1191 | E₁ₐE₂E₃EᵢᵢᵢE₄ | | | E_{d} |
| 1192 | E_{1b}E₂E₃EᵢᵢᵢE₄ | | | E_{d} |
| 1193 | E₁ₐE₂Eᵢᵥ | | | E_{d} |
| 1194 | E_{1b}E₂Eᵢᵥ | | | E_{d} |
| 1195 | E₁ₐE₂E₃Eᵢᵥ | | | E_{d} |
| 1196 | E_{1b}E₂E₃Eᵢᵥ | | | E_{d} |
| 1197 | E₁ₐE₂EᵢᵥE₄ | | | E_{d} |
| 1198 | E_{1b}E₂EᵢᵥE₄ | | | E_{d} |
| 1199 | E₁ₐE₂E₃EᵢᵥE₄ | | | E_{d} |
| 1200 | E_{1b}E₂E₃EᵢᵥE₄ | | | E_{d} |
| 1201 | E₁ₐE₂ | | E₄* | E_{d} |
| 1202 | E_{1b}E₂ | | E₄* | E_{d} |
| 1203 | E₁ₐE₂E₃ | | E₄* | E_{d} |
| 1204 | E_{1b}E₂E₃ | | E₄* | E_{d} |
| 1205 | E₁ₐE₂Eᵢ | | E₄* | E_{d} |
| 1206 | E_{1b}E₂Eᵢ | | E₄* | E_{d} |
| 1207 | E₁ₐE₂Eᵢᵢ | | E₄* | E_{d} |
| 1208 | E_{1b}E₂Eᵢᵢ | | E₄* | E_{d} |
| 1209 | E₁ₐE₂EᵢᵢE_{iib} | | E₄* | E_{d} |
| 1210 | E_{1b}E₂EᵢᵢE_{iib} | | E₄* | E_{d} |
| 1211 | E₁ₐE₂E₃Eᵢ | | E₄* | E_{d} |
| 1212 | E_{1b}E₂E₃Eᵢ | | E₄* | E_{d} |
| 1213 | E₁ₐE₂E₃Eᵢᵢ | | E₄* | E_{d} |
| 1214 | E_{1b}E₂E₃Eᵢᵢ | | E₄* | E_{d} |
| 1215 | E₁ₐE₂E₃EᵢᵢE_{iib} | | E₄* | E_{d} |
| 1216 | E_{1b}E₂E₃EᵢᵢE_{iib} | | E₄* | E_{d} |
| 1217 | E₁ₐE₂E₄ | | E₄* | E_{d} |
| 1218 | E_{1b}E₂E₄ | | E₄* | E_{d} |
| 1219 | E₁ₐE₂E₃E₄ | | E₄* | E_{d} |
| 1220 | E_{1b}E₂E₃E₄ | | E₄* | E_{d} |
| 1221 | E₁ₐE₂EᵢE₄ | | E₄* | E_{d} |
| 1222 | E_{1b}E₂EᵢE₄ | | E₄* | E_{d} |
| 1223 | E₁ₐE₂EᵢᵢE₄ | | E₄* | E_{d} |
| 1224 | E_{1b}E₂EᵢᵢE₄ | | E₄* | E_{d} |
| 1225 | E₁ₐE₂EᵢᵢE_{iib}E₄ | | E₄* | E_{d} |
| 1226 | E_{1b}E₂EᵢᵢE_{iib}E₄ | | E₄* | E_{d} |
| 1227 | E₁ₐE₂E₃EᵢE₄ | | E₄* | E_{d} |
| 1228 | E_{1b}E₂E₃EᵢE₄ | | E₄* | E_{d} |
| 1229 | E₁ₐE₂E₃EᵢᵢE₄ | | E₄* | E_{d} |
| 1230 | E_{1b}E₂E₃EᵢᵢE₄ | | E₄* | E_{d} |
| 1231 | E₁ₐE₂E₃EᵢᵢE_{iib}E₄ | | E₄* | E_{d} |
| 1232 | E_{1b}E₂E₃EᵢᵢE_{iib}E₄ | | E₄* | E_{d} |
| 1233 | E₁ₐE₂Eᵢᵢᵢ | | E₄* | E_{d} |
| 1234 | E_{1b}E₂Eᵢᵢᵢ | | E₄* | E_{d} |
| 1235 | E₁ₐE₂E₃Eᵢᵢᵢ | | E₄* | E_{d} |
| 1236 | E_{1b}E₂E₃Eᵢᵢᵢ | | E₄* | E_{d} |
| 1237 | E₁ₐE₂EᵢᵢᵢE₄ | | E₄* | E_{d} |
| 1238 | E_{1b}E₂EᵢᵢᵢE₄ | | E₄* | E_{d} |
| 1239 | E₁ₐE₂E₃EᵢᵢᵢE₄ | | E₄* | E_{d} |
| 1240 | E_{1b}E₂E₃EᵢᵢᵢE₄ | | E₄* | E_{d} |
| 1241 | E₁ₐE₂Eᵢᵥ | | E₄* | E_{d} |
| 1242 | E_{1b}E₂Eᵢᵥ | | E₄* | E_{d} |
| 1243 | E₁ₐE₂E₃Eᵢᵥ | | E₄* | E_{d} |
| 1244 | E_{1b}E₂E₃Eᵢᵥ | | E₄* | E_{d} |
| 1245 | E₁ₐE₂EᵢᵥE₄ | | E₄* | E_{d} |
| 1246 | E_{1b}E₂EᵢᵥE₄ | | E₄* | E_{d} |
| 1247 | E₁ₐE₂E₃EᵢᵥE₄ | | E₄* | E_{d} |
| 1248 | E_{1b}E₂E₃EᵢᵥE₄ | | E₄* | E_{d} |
| 1249 | E₁ₐE₂ | | E₅ | E_{d} |
| 1250 | E_{1b}E₂ | | E₅ | E_{d} |
| 1251 | E₁ₐE₂E₃ | | E₅ | E_{d} |
| 1252 | E_{1b}E₂E₃ | | E₅ | E_{d} |
| 1253 | E₁ₐE₂Eᵢ | | E₅ | E_{d} |
| 1254 | E_{1b}E₂Eᵢ | | E₅ | E_{d} |
| 1255 | E₁ₐE₂Eᵢᵢ | | E₅ | E_{d} |
| 1256 | E_{1b}E₂Eᵢᵢ | | E₅ | E_{d} |
| 1257 | E₁ₐE₂EᵢᵢE_{iib} | | E₅ | E_{d} |
| 1258 | E_{1b}E₂EᵢᵢE_{iib} | | E₅ | E_{d} |
| 1259 | E₁ₐE₂E₃Eᵢ | | E₅ | E_{d} |
| 1260 | E_{1b}E₂E₃Eᵢ | | E₅ | E_{d} |
| 1261 | E₁ₐE₂E₃Eᵢᵢ | | E₅ | E_{d} |
| 1262 | E_{1b}E₂E₃Eᵢᵢ | | E₅ | E_{d} |
| 1263 | E₁ₐE₂E₃EᵢᵢE_{iib} | | E₅ | E_{d} |
| 1264 | E_{1b}E₂E₃EᵢᵢE_{iib} | | E₅ | E_{d} |
| 1265 | E₁ₐE₂E₄ | | E₅ | E_{d} |
| 1266 | E_{1b}E₂E₄ | | E₅ | E_{d} |
| 1267 | E₁ₐE₂E₃E₄ | | E₅ | E_{d} |
| 1268 | E_{1b}E₂E₃E₄ | | E₅ | E_{d} |
| 1269 | E₁ₐE₂EᵢE₄ | | E₅ | E_{d} |
| 1270 | E_{1b}E₂EᵢE₄ | | E₅ | E_{d} |
| 1271 | E₁ₐE₂EᵢᵢE₄ | | E₅ | E_{d} |
| 1272 | E_{1b}E₂EᵢᵢE₄ | | E₅ | E_{d} |
| 1273 | E₁ₐE₂EᵢᵢE_{iib}E₄ | | E₅ | E_{d} |
| 1274 | E_{1b}E₂EᵢᵢE_{iib}E₄ | | E₅ | E_{d} |
| 1275 | E₁ₐE₂E₃EᵢE₄ | | E₅ | E_{d} |
| 1276 | E_{1b}E₂E₃EᵢE₄ | | E₅ | E_{d} |
| 1277 | E₁ₐE₂E₃EᵢᵢE₄ | | E₅ | E_{d} |
| 1278 | E_{1b}E₂E₃EᵢᵢE₄ | | E₅ | E_{d} |
| 1279 | E₁ₐE₂E₃EᵢᵢE_{iib}E₄ | | E₅ | E_{d} |
| 1280 | E_{1b}E₂E₃EᵢᵢE_{iib}E₄ | | E₅ | E_{d} |
| 1281 | E₁ₐE₂Eᵢᵢᵢ | | E₅ | E_{d} |
| 1282 | E_{1b}E₂Eᵢᵢᵢ | | E₅ | E_{d} |
| 1283 | E₁ₐE₂E₃Eᵢᵢᵢ | | E₅ | E_{d} |
| 1284 | E_{1b}E₂E₃Eᵢᵢᵢ | | E₅ | E_{d} |
| 1285 | E₁ₐE₂EᵢᵢᵢE₄ | | E₅ | E_{d} |
| 1286 | E_{1b}E₂EᵢᵢᵢE₄ | | E₅ | E_{d} |
| 1287 | E₁ₐE₂E₃EᵢᵢᵢE₄ | | E₅ | E_{d} |
| 1288 | E_{1b}E₂E₃EᵢᵢᵢE₄ | | E₅ | E_{d} |
| 1289 | E₁ₐE₂Eᵢᵥ | | E₅ | E_{d} |
| 1290 | E_{1b}E₂Eᵢᵥ | | E₅ | E_{d} |
| 1291 | E₁ₐE₂E₃Eᵢᵥ | | E₅ | E_{d} |
| 1292 | E_{1b}E₂E₃Eᵢᵥ | | E₅ | E_{d} |
| 1293 | E₁ₐE₂EᵢᵥE₄ | | E₅ | E_{d} |
| 1294 | E_{1b}E₂EᵢᵥE₄ | | E₅ | E_{d} |
| 1295 | E₁ₐE₂E₃EᵢᵥE₄ | | E₅ | E_{d} |
| 1296 | E_{1b}E₂E₃EᵢᵥE₄ | | E₅ | E_{d} |
| 1297 | E₁ₐE₂ | | E₄*E₅ | E_{d} |
| 1298 | E_{1b}E₂ | | E₄*E₅ | E_{d} |
| 1299 | E₁ₐE₂E₃ | | E₄*E₅ | E_{d} |
| 1300 | E_{1b}E₂E₃ | | E₄*E₅ | E_{d} |
| 1301 | E₁ₐE₂Eᵢ | | E₄*E₅ | E_{d} |
| 1302 | E_{1b}E₂Eᵢ | | E₄*E₅ | E_{d} |
| 1303 | E₁ₐE₂Eᵢᵢ | | E₄*E₅ | E_{d} |
| 1304 | E_{1b}E₂Eᵢᵢ | | E₄*E₅ | E_{d} |
| 1305 | E₁ₐE₂EᵢᵢE_{iib} | | E₄*E₅ | E_{d} |
| 1306 | E_{1b}E₂EᵢᵢE_{iib} | | E₄*E₅ | E_{d} |
| 1307 | E₁ₐE₂E₃Eᵢ | | E₄*E₅ | E_{d} |
| 1308 | E_{1b}E₂E₃Eᵢ | | E₄*E₅ | E_{d} |
| 1309 | E₁ₐE₂E₃Eᵢᵢ | | E₄*E₅ | E_{d} |
| 1310 | E_{1b}E₂E₃Eᵢᵢ | | E₄*E₅ | E_{d} |
| 1311 | E₁ₐE₂E₃EᵢᵢE_{iib} | | E₄*E₅ | E_{d} |
| 1312 | E_{1b}E₂E₃EᵢᵢE_{iib} | | E₄*E₅ | E_{d} |
| 1313 | E₁ₐE₂E₄ | | E₄*E₅ | E_{d} |
| 1314 | E_{1b}E₂E₄ | | E₄*E₅ | E_{d} |
| 1315 | E₁ₐE₂E₃E₄ | | E₄*E₅ | E_{d} |
| 1316 | E_{1b}E₂E₃E₄ | | E₄*E₅ | E_{d} |
| 1317 | E₁ₐE₂EᵢE₄ | | E₄*E₅ | E_{d} |
| 1318 | E_{1b}E₂EᵢE₄ | | E₄*E₅ | E_{d} |
| 1319 | E₁ₐE₂EᵢᵢE₄ | | E₄*E₅ | E_{d} |
| 1320 | E_{1b}E₂EᵢᵢE₄ | | E₄*E₅ | E_{d} |
| 1321 | E₁ₐE₂EᵢᵢE_{iib}E₄ | | E₄*E₅ | E_{d} |
| 1322 | E_{1b}E₂EᵢᵢE_{iib}E₄ | | E₄*E₅ | E_{d} |
| 1323 | E₁ₐE₂E₃EᵢE₄ | | E₄*E₅ | E_{d} |
| 1324 | E_{1b}E₂E₃EᵢE₄ | | E₄*E₅ | E_{d} |
| 1325 | E₁ₐE₂E₃EᵢᵢE₄ | | E₄*E₅ | E_{d} |
| 1326 | E_{1b}E₂E₃EᵢᵢE₄ | | E₄*E₅ | E_{d} |
| 1327 | E₁ₐE₂E₃EᵢᵢE_{iib}E₄ | | E₄*E₅ | E_{d} |
| 1328 | E_{1b}E₂E₃EᵢᵢE_{iib}E₄ | | E₄*E₅ | E_{d} |
| 1329 | E₁ₐE₂Eᵢᵢᵢ | | E₄*E₅ | E_{d} |
| 1330 | E_{1b}E₂Eᵢᵢᵢ | | E₄*E₅ | E_{d} |
| 1331 | E₁ₐE₂E₃Eᵢᵢᵢ | | E₄*E₅ | E_{d} |
| 1332 | E_{1b}E₂E₃Eᵢᵢᵢ | | E₄*E₅ | E_{d} |
| 1333 | E₁ₐE₂EᵢᵢᵢE₄ | | E₄*E₅ | E_{d} |
| 1334 | E_{1b}E₂EᵢᵢᵢE₄ | | E₄*E₅ | E_{d} |
| 1335 | E₁ₐE₂E₃EᵢᵢᵢE₄ | | E₄*E₅ | E_{d} |
| 1336 | E_{1b}E₂E₃EᵢᵢᵢE₄ | | E₄*E₅ | E_{d} |
| 1337 | E₁ₐE₂Eᵢᵥ | | E₄*E₅ | E_{d} |
| 1338 | E_{1b}E₂Eᵢᵥ | | E₄*E₅ | E_{d} |
| 1339 | E₁ₐE₂E₃Eᵢᵥ | | E₄*E₅ | E_{d} |
| 1340 | E_{1b}E₂E₃Eᵢᵥ | | E₄*E₅ | E_{d} |
| 1341 | E₁ₐE₂EᵢᵥE₄ | | E₄*E₅ | E_{d} |
| 1342 | E_{1b}E₂EᵢᵥE₄ | | E₄*E₅ | E_{d} |
| 1343 | E₁ₐE₂E₃EᵢᵥE₄ | | E₄*E₅ | E_{d} |
| 1344 | E_{1b}E₂E₃EᵢᵥE₄ | | E₄*E₅ | E_{d} |
| 1345 | E₁ₐE₂ | AlkL | | E_{d} |
| 1346 | E_{1b}E₂ | AlkL | | E_{d} |
| 1347 | E₁ₐE₂E₃ | AlkL | | E_{d} |
| 1348 | E_{1b}E₂E₃ | AlkL | | E_{d} |
| 1349 | E₁ₐE₂Eᵢ | AlkL | | E_{d} |
| 1350 | E_{1b}E₂Eᵢ | AlkL | | E_{d} |
| 1351 | E₁ₐE₂Eᵢᵢ | AlkL | | E_{d} |
| 1352 | E_{1b}E₂Eᵢᵢ | AlkL | | E_{d} |
| 1353 | E₁ₐE₂EᵢᵢE_{iib} | AlkL | | E_{d} |
| 1354 | E_{1b}E₂EᵢᵢE_{iib} | AlkL | | E_{d} |
| 1355 | E₁ₐE₂E₃Eᵢ | AlkL | | E_{d} |
| 1356 | E_{1b}E₂E₃Eᵢ | AlkL | | E_{d} |
| 1357 | E₁ₐE₂E₃Eᵢᵢ | AlkL | | E_{d} |
| 1358 | E_{1b}E₂E₃Eᵢᵢ | AlkL | | E_{d} |
| 1359 | E₁ₐE₂E₃EᵢᵢE_{iib} | AlkL | | E_{d} |
| 1360 | E_{1b}E₂E₃EᵢᵢE_{iib} | AlkL | | E_{d} |
| 1361 | E₁ₐE₂E₄ | AlkL | | E_{d} |
| 1362 | E_{1b}E₂E₄ | AlkL | | E_{d} |
| 1363 | E₁ₐE₂E₃E₄ | AlkL | | E_{d} |
| 1364 | E_{1b}E₂E₃E₄ | AlkL | | E_{d} |
| 1365 | E₁ₐE₂EᵢE₄ | AlkL | | E_{d} |
| 1366 | E_{1b}E₂EᵢE₄ | AlkL | | E_{d} |
| 1367 | E₁ₐE₂EᵢᵢE₄ | AlkL | | E_{d} |
| 1368 | E_{1b}E₂EᵢᵢE₄ | AlkL | | E_{d} |
| 1369 | E₁ₐE₂EᵢᵢE_{iib}E₄ | AlkL | | E_{d} |
| 1370 | E_{1b}E₂EᵢᵢE_{iib}E₄ | AlkL | | E_{d} |
| 1371 | E₁ₐE₂E₃EᵢE₄ | AlkL | | E_{d} |
| 1372 | E_{1b}E₂E₃EᵢE₄ | AlkL | | E_{d} |
| 1373 | E₁ₐE₂E₃EᵢᵢE₄ | AlkL | | E_{d} |
| 1374 | E_{1b}E₂E₃EᵢᵢE₄ | AlkL | | E_{d} |
| 1375 | E₁ₐE₂E₃EᵢᵢE_{iib}E₄ | AlkL | | E_{d} |
| 1376 | E_{1b}E₂E₃EᵢᵢE_{iib}E₄ | AlkL | | E_{d} |
| 1377 | E₁ₐE₂Eᵢᵢᵢ | AlkL | | E_{d} |
| 1378 | E_{1b}E₂Eᵢᵢᵢ | AlkL | | E_{d} |
| 1379 | E₁ₐE₂E₃Eᵢᵢᵢ | AlkL | | E_{d} |
| 1380 | E_{1b}E₂E₃Eᵢᵢᵢ | AlkL | | E_{d} |
| 1381 | E₁ₐE₂EᵢᵢᵢE₄ | AlkL | | E_{d} |
| 1382 | E_{1b}E₂EᵢᵢᵢE₄ | AlkL | | E_{d} |
| 1383 | E₁ₐE₂E₃EᵢᵢᵢE₄ | AlkL | | E_{d} |
| 1384 | E_{1b}E₂E₃EᵢᵢᵢE₄ | AlkL | | E_{d} |
| 1385 | E₁ₐE₂Eᵢᵥ | AlkL | | E_{d} |
| 1386 | E_{1b}E₂Eᵢᵥ | AlkL | | E_{d} |
| 1387 | E₁ₐE₂E₃Eᵢᵥ | AlkL | | E_{d} |
| 1388 | E_{1b}E₂E₃Eᵢᵥ | AlkL | | E_{d} |
| 1389 | E₁ₐE₂EᵢᵥE₄ | AlkL | | E_{d} |
| 1390 | E_{1b}E₂EᵢᵥE₄ | AlkL | | E_{d} |
| 1391 | E₁ₐE₂E₃EᵢᵥE₄ | AlkL | | E_{d} |
| 1392 | E_{1b}E₂E₃EᵢᵥE₄ | AlkL | | E_{d} |
| 1393 | E₁ₐE₂ | AlkL | E₄* | E_{d} |
| 1394 | E_{1b}E₂ | AlkL | E₄* | E_{d} |
| 1395 | E₁ₐE₂E₃ | AlkL | E₄* | E_{d} |
| 1396 | E_{1b}E₂E₃ | AlkL | E₄* | E_{d} |
| 1397 | E₁ₐE₂Eᵢ | AlkL | E₄* | E_{d} |
| 1398 | E_{1b}E₂Eᵢ | AlkL | E₄* | E_{d} |
| 1399 | E₁ₐE₂Eᵢᵢ | AlkL | E₄* | E_{d} |
| 1400 | E_{1b}E₂Eᵢᵢ | AlkL | E₄* | E_{d} |
| 1401 | E₁ₐE₂EᵢᵢE_{iib} | AlkL | E₄* | E_{d} |
| 1402 | E_{1b}E₂EᵢᵢE_{iib} | AlkL | E₄* | E_{d} |
| 1403 | E₁ₐE₂E₃Eᵢ | AlkL | E₄* | E_{d} |
| 1404 | E_{1b}E₂E₃Eᵢ | AlkL | E₄* | E_{d} |
| 1405 | E₁ₐE₂E₃Eᵢᵢ | AlkL | E₄* | E_{d} |
| 1406 | E_{1b}E₂E₃Eᵢᵢ | AlkL | E₄* | E_{d} |
| 1407 | E₁ₐE₂E₃EᵢᵢE_{iib} | AlkL | E₄* | E_{d} |
| 1408 | E_{1b}E₂E₃EᵢᵢE_{iib} | AlkL | E₄* | E_{d} |
| 1409 | E₁ₐE₂E₄ | AlkL | E₄* | E_{d} |
| 1410 | E_{1b}E₂E₄ | AlkL | E₄* | E_{d} |
| 1411 | E₁ₐE₂E₃E₄ | AlkL | E₄* | E_{d} |
| 1412 | E_{1b}E₂E₃E₄ | AlkL | E₄* | E_{d} |
| 1413 | E₁ₐE₂EᵢE₄ | AlkL | E₄* | E_{d} |
| 1414 | E_{1b}E₂EᵢE₄ | AlkL | E₄* | E_{d} |
| 1415 | E₁ₐE₂EᵢᵢE₄ | AlkL | E₄* | E_{d} |
| 1416 | E_{1b}E₂EᵢᵢE₄ | AlkL | E₄* | E_{d} |
| 1417 | E₁ₐE₂EᵢᵢE_{iib}E₄ | AlkL | E₄* | E_{d} |
| 1418 | E_{1b}E₂EᵢᵢE_{iib}E₄ | AlkL | E₄* | E_{d} |
| 1419 | E₁ₐE₂E₃EᵢE₄ | AlkL | E₄* | E_{d} |
| 1420 | E_{1b}E₂E₃EᵢE₄ | AlkL | E₄* | E_{d} |
| 1421 | E₁ₐE₂E₃EᵢᵢE₄ | AlkL | E₄* | E_{d} |
| 1422 | E_{1b}E₂E₃EᵢᵢE₄ | AlkL | E₄* | E_{d} |
| 1423 | E₁ₐE₂E₃EᵢᵢE_{iib}E₄ | AlkL | E₄* | E_{d} |
| 1424 | E_{1b}E₂E₃EᵢᵢE_{iib}E₄ | AlkL | E₄* | E_{d} |
| 1425 | E₁ₐE₂Eᵢᵢᵢ | AlkL | E₄* | E_{d} |
| 1426 | E_{1b}E₂Eᵢᵢᵢ | AlkL | E₄* | E_{d} |
| 1427 | E₁ₐE₂E₃Eᵢᵢᵢ | AlkL | E₄* | E_{d} |
| 1428 | E_{1b}E₂E₃Eᵢᵢᵢ | AlkL | E₄* | E_{d} |
| 1429 | E₁ₐE₂EᵢᵢᵢE₄ | AlkL | E₄* | E_{d} |
| 1430 | E_{1b}E₂EᵢᵢᵢE₄ | AlkL | E₄* | E_{d} |
| 1431 | E₁ₐE₂E₃EᵢᵢᵢE₄ | AlkL | E₄* | E_{d} |
| 1432 | E_{1b}E₂E₃EᵢᵢᵢE₄ | AlkL | E₄* | E_{d} |
| 1433 | E₁ₐE₂Eᵢᵥ | AlkL | E₄* | E_{d} |
| 1434 | E_{1b}E₂Eᵢᵥ | AlkL | E₄* | E_{d} |
| 1435 | E₁ₐE₂E₃Eᵢᵥ | AlkL | E₄* | E_{d} |
| 1436 | E_{1b}E₂E₃Eᵢᵥ | AlkL | E₄* | E_{d} |
| 1437 | E₁ₐE₂EᵢᵥE₄ | AlkL | E₄* | E_{d} |
| 1438 | E_{1b}E₂EᵢᵥE₄ | AlkL | E₄* | E_{d} |
| 1439 | E₁ₐE₂E₃EᵢᵥE₄ | AlkL | E₄* | E_{d} |
| 1440 | E_{1b}E₂E₃EᵢᵥE₄ | AlkL | E₄* | E_{d} |
| 1441 | E₁ₐE₂ | AlkL | E₅ | E_{d} |
| 1442 | E_{1b}E₂ | AlkL | E₅ | E_{d} |
| 1443 | E₁ₐE₂E₃ | AlkL | E₅ | E_{d} |
| 1444 | E_{1b}E₂E₃ | AlkL | E₅ | E_{d} |
| 1445 | E₁ₐE₂Eᵢ | AlkL | E₅ | E_{d} |
| 1446 | E_{1b}E₂Eᵢ | AlkL | E₅ | E_{d} |
| 1447 | E₁ₐE₂Eᵢᵢ | AlkL | E₅ | E_{d} |
| 1448 | E_{1b}E₂Eᵢᵢ | AlkL | E₅ | E_{d} |
| 1449 | E₁ₐE₂EᵢᵢE_{iib} | AlkL | E₅ | E_{d} |
| 1450 | E_{1b}E₂EᵢᵢE_{iib} | AlkL | E₅ | E_{d} |
| 1451 | E₁ₐE₂E₃Eᵢ | AlkL | E₅ | E_{d} |
| 1452 | E_{1b}E₂E₃Eᵢ | AlkL | E₅ | E_{d} |
| 1453 | E₁ₐE₂E₃Eᵢᵢ | AlkL | E₅ | E_{d} |
| 1454 | E_{1b}E₂E₃Eᵢᵢ | AlkL | E₅ | E_{d} |
| 1455 | E₁ₐE₂E₃EᵢᵢE_{iib} | AlkL | E₅ | E_{d} |
| 1456 | E_{1b}E₂E₃EᵢᵢE_{iib} | AlkL | E₅ | E_{d} |
| 1457 | E₁ₐE₂E₄ | AlkL | E₅ | E_{d} |
| 1458 | E_{1b}E₂E₄ | AlkL | E₅ | E_{d} |
| 1459 | E₁ₐE₂E₃E₄ | AlkL | E₅ | E_{d} |
| 1460 | E_{1b}E₂E₃E₄ | AlkL | E₅ | E_{d} |
| 1461 | E₁ₐE₂EᵢE₄ | AlkL | E₅ | E_{d} |
| 1462 | E_{1b}E₂EᵢE₄ | AlkL | E₅ | E_{d} |
| 1463 | E₁ₐE₂EᵢᵢE₄ | AlkL | E₅ | E_{d} |
| 1464 | E_{1b}E₂EᵢᵢE₄ | AlkL | E₅ | E_{d} |
| 1465 | E₁ₐE₂EᵢᵢE_{iib}E₄ | AlkL | E₅ | E_{d} |
| 1466 | E_{1b}E₂EᵢᵢE_{iib}E₄ | AlkL | E₅ | E_{d} |
| 1467 | E₁ₐE₂E₃EᵢE₄ | AlkL | E₅ | E_{d} |
| 1468 | E_{1b}E₂E₃EᵢE₄ | AlkL | E₅ | E_{d} |
| 1469 | E₁ₐE₂E₃EᵢᵢE₄ | AlkL | E₅ | E_{d} |
| 1470 | E_{1b}E₂E₃EᵢᵢE₄ | AlkL | E₅ | E_{d} |
| 1471 | E₁ₐE₂E₃EᵢᵢE_{iib}E₄ | AlkL | E₅ | E_{d} |
| 1472 | E_{1b}E₂E₃EᵢᵢE_{iib}E₄ | AlkL | E₅ | E_{d} |
| 1473 | E₁ₐE₂Eᵢᵢᵢ | AlkL | E₅ | E_{d} |
| 1474 | E_{1b}E₂Eᵢᵢᵢ | AlkL | E₅ | E_{d} |
| 1475 | E₁ₐE₂E₃Eᵢᵢᵢ | AlkL | E₅ | E_{d} |
| 1476 | E_{1b}E₂E₃Eᵢᵢᵢ | AlkL | E₅ | E_{d} |
| 1477 | E₁ₐE₂EᵢᵢᵢE₄ | AlkL | E₅ | E_{d} |
| 1478 | E_{1b}E₂EᵢᵢᵢE₄ | AlkL | E₅ | E_{d} |
| 1479 | E₁ₐE₂E₃EᵢᵢᵢE₄ | AlkL | E₅ | E_{d} |
| 1480 | E_{1b}E₂E₃EᵢᵢᵢE₄ | AlkL | E₅ | E_{d} |
| 1481 | E₁ₐE₂Eᵢᵥ | AlkL | E₅ | E_{d} |
| 1482 | E_{1b}E₂Eᵢᵥ | AlkL | E₅ | E_{d} |
| 1483 | E₁ₐE₂E₃Eᵢᵥ | AlkL | E₅ | E_{d} |
| 1484 | E_{1b}E₂E₃Eᵢᵥ | AlkL | E₅ | E_{d} |
| 1485 | E₁ₐE₂EᵢᵥE₄ | AlkL | E₅ | E_{d} |
| 1486 | E_{1b}E₂EᵢᵥE₄ | AlkL | E₅ | E_{d} |
| 1487 | E₁ₐE₂E₃EᵢᵥE₄ | AlkL | E₅ | E_{d} |
| 1488 | E_{1b}E₂E₃EᵢᵥE₄ | AlkL | E₅ | E_{d} |
| 1489 | E₁ₐE₂ | AlkL | E₄*E₅ | E_{d} |
| 1490 | E_{1b}E₂ | AlkL | E₄*E₅ | E_{d} |
| 1491 | E₁ₐE₂E₃ | AlkL | E₄*E₅ | E_{d} |
| 1492 | E_{1b}E₂E₃ | AlkL | E₄*E₅ | E_{d} |
| 1493 | E₁ₐE₂Eᵢ | AlkL | E₄*E₅ | E_{d} |
| 1494 | E_{1b}E₂Eᵢ | AlkL | E₄*E₅ | E_{d} |
| 1495 | E₁ₐE₂Eᵢᵢ | AlkL | E₄*E₅ | E_{d} |
| 1496 | E_{1b}E₂Eᵢᵢ | AlkL | E₄*E₅ | E_{d} |
| 1497 | E₁ₐE₂EᵢᵢE_{iib} | AlkL | E₄*E₅ | E_{d} |
| 1498 | E_{1b}E₂EᵢᵢE_{iib} | AlkL | E₄*E₅ | E_{d} |
| 1499 | E₁ₐE₂E₃Eᵢ | AlkL | E₄*E₅ | E_{d} |
| 1500 | E_{1b}E₂E₃Eᵢ | AlkL | E₄*E₅ | E_{d} |
| 1501 | E₁ₐE₂E₃Eᵢᵢ | AlkL | E₄*E₅ | E_{d} |
| 1502 | E_{1b}E₂E₃Eᵢᵢ | AlkL | E₄*E₅ | E_{d} |
| 1503 | E₁ₐE₂E₃EᵢᵢE_{iib} | AlkL | E₄*E₅ | E_{d} |
| 1504 | E_{1b}E₂E₃EᵢᵢE_{iib} | AlkL | E₄*E₅ | E_{d} |
| 1505 | E₁ₐE₂E₄ | AlkL | E₄*E₅ | E_{d} |
| 1506 | E_{1b}E₂E₄ | AlkL | E₄*E₅ | E_{d} |
| 1507 | E₁ₐE₂E₃E₄ | AlkL | E₄*E₅ | E_{d} |
| 1508 | E_{1b}E₂E₃E₄ | AlkL | E₄*E₅ | E_{d} |
| 1509 | E₁ₐE₂EᵢE₄ | AlkL | E₄*E₅ | E_{d} |
| 1510 | E_{1b}E₂EᵢE₄ | AlkL | E₄*E₅ | E_{d} |
| 1511 | E₁ₐE₂EᵢᵢE₄ | AlkL | E₄*E₅ | E_{d} |
| 1512 | E_{1b}E₂EᵢᵢE₄ | AlkL | E₄*E₅ | E_{d} |
| 1513 | E₁ₐE₂EᵢᵢE_{iib}E₄ | AlkL | E₄*E₅ | E_{d} |
| 1514 | E_{1b}E₂EᵢᵢE_{iib}E₄ | AlkL | E₄*E₅ | E_{d} |
| 1515 | E₁ₐE₂E₃EᵢE₄ | AlkL | E₄*E₅ | E_{d} |
| 1516 | E_{1b}E₂E₃EᵢE₄ | AlkL | E₄*E₅ | E_{d} |
| 1517 | E₁ₐE₂E₃EᵢᵢE₄ | AlkL | E₄*E₅ | E_{d} |
| 1518 | E_{1b}E₂E₃EᵢᵢE₄ | AlkL | E₄*E₅ | E_{d} |
| 1519 | E₁ₐE₂E₃EᵢᵢE_{iib}E₄ | AlkL | E₄*E₅ | E_{d} |
| 1520 | E_{1b}E₂E₃EᵢᵢE_{iib}E₄ | AlkL | E₄*E₅ | E_{d} |
| 1521 | E₁ₐE₂Eᵢᵢᵢ | AlkL | E₄*E₅ | E_{d} |
| 1522 | E_{1b}E₂Eᵢᵢᵢ | AlkL | E₄*E₅ | E_{d} |
| 1523 | E₁ₐE₂E₃Eᵢᵢᵢ | AlkL | E₄*E₅ | E_{d} |
| 1524 | E_{1b}E₂E₃Eᵢᵢᵢ | AlkL | E₄*E₅ | E_{d} |
| 1525 | E₁ₐE₂EᵢᵢᵢE₄ | AlkL | E₄*E₅ | E_{d} |
| 1526 | E_{1b}E₂EᵢᵢᵢE₄ | AlkL | E₄*E₅ | E_{d} |
| 1527 | E₁ₐE₂E₃EᵢᵢᵢE₄ | AlkL | E₄*E₅ | E_{d} |
| 1528 | E_{1b}E₂E₃EᵢᵢᵢE₄ | AlkL | E₄*E₅ | E_{d} |
| 1529 | E₁ₐE₂Eᵢᵥ | AlkL | E₄*E₅ | E_{d} |
| 1530 | E_{1b}E₂Eᵢᵥ | AlkL | E₄*E₅ | E_{d} |
| 1531 | E₁ₐE₂E₃Eᵢᵥ | AlkL | E₄*E₅ | E_{d} |
| 1532 | E_{1b}E₂E₃Eᵢᵥ | AlkL | E₄*E₅ | E_{d} |
| 1533 | E₁ₐE₂EᵢᵥE₄ | AlkL | E₄*E₅ | E_{d} |
| 1534 | E_{1b}E₂EᵢᵥE₄ | AlkL | E₄*E₅ | E_{d} |
| 1535 | E₁ₐE₂E₃EᵢᵥE₄ | AlkL | E₄*E₅ | E_{d} |
| 1536 | E_{1b}E₂E₃EᵢᵥE₄ | AlkL | E₄*E₅ | E_{d} |
| 1537 | E₁ₐE₂ | | | Eₑ |
| 1538 | E_{1b}E₂ | | | Eₑ |
| 1539 | E₁ₐE₂E₃ | | | Eₑ |
| 1540 | E_{1b}E₂E₃ | | | Eₑ |
| 1541 | E₁ₐE₂Eᵢ | | | Eₑ |
| 1542 | E_{1b}E₂Eᵢ | | | Eₑ |
| 1543 | E₁ₐE₂Eᵢᵢ | | | Eₑ |
| 1544 | E_{1b}E₂Eᵢᵢ | | | Eₑ |
| 1545 | E₁ₐE₂EᵢᵢE_{iib} | | | Eₑ |
| 1546 | E_{1b}E₂EᵢᵢE_{iib} | | | Eₑ |
| 1547 | E₁ₐE₂E₃Eᵢ | | | Eₑ |
| 1548 | E_{1b}E₂E₃Eᵢ | | | Eₑ |
| 1549 | E₁ₐE₂E₃Eᵢᵢ | | | Eₑ |
| 1550 | E_{1b}E₂E₃Eᵢᵢ | | | Eₑ |
| 1551 | E₁ₐE₂E₃EᵢᵢE_{iib} | | | Eₑ |
| 1552 | E_{1b}E₂E₃EᵢᵢE_{iib} | | | Eₑ |
| 1553 | E₁ₐE₂E₄ | | | Eₑ |
| 1554 | E_{1b}E₂E₄ | | | Eₑ |
| 1555 | E₁ₐE₂E₃E₄ | | | Eₑ |
| 1556 | E_{1b}E₂E₃E₄ | | | Eₑ |
| 1557 | E₁ₐE₂EᵢE₄ | | | Eₑ |
| 1558 | E_{1b}E₂EᵢE₄ | | | Eₑ |
| 1559 | E₁ₐE₂EᵢᵢE₄ | | | Eₑ |
| 1560 | E_{1b}E₂EᵢᵢE₄ | | | Eₑ |
| 1561 | E₁ₐE₂EᵢᵢE_{iib}E₄ | | | Eₑ |
| 1562 | E_{1b}E₂EᵢᵢE_{iib}E₄ | | | Eₑ |
| 1563 | E₁ₐE₂E₃EᵢE₄ | | | Eₑ |
| 1564 | E_{1b}E₂E₃EᵢE₄ | | | Eₑ |
| 1565 | E₁ₐE₂E₃EᵢᵢE₄ | | | Eₑ |
| 1566 | E_{1b}E₂E₃EᵢᵢE₄ | | | Eₑ |
| 1567 | E₁ₐE₂E₃EᵢᵢE_{iib}E₄ | | | Eₑ |
| 1568 | E_{1b}E₂E₃EᵢᵢE_{iib}E₄ | | | Eₑ |
| 1569 | E₁ₐE₂Eᵢᵢᵢ | | | Eₑ |
| 1570 | E_{1b}E₂Eᵢᵢᵢ | | | Eₑ |
| 1571 | E₁ₐE₂E₃Eᵢᵢᵢ | | | Eₑ |
| 1572 | E_{1b}E₂E₃Eᵢᵢᵢ | | | Eₑ |
| 1573 | E₁ₐE₂EᵢᵢᵢE₄ | | | Eₑ |
| 1574 | E_{1b}E₂EᵢᵢᵢE₄ | | | Eₑ |
| 1575 | E₁ₐE₂E₃EᵢᵢᵢE₄ | | | Eₑ |
| 1576 | E_{1b}E₂E₃EᵢᵢᵢE₄ | | | Eₑ |
| 1577 | E₁ₐE₂Eᵢᵥ | | | Eₑ |
| 1578 | E_{1b}E₂Eᵢᵥ | | | Eₑ |
| 1579 | E₁ₐE₂E₃Eᵢᵥ | | | Eₑ |
| 1580 | E_{1b}E₂E₃Eᵢᵥ | | | Eₑ |
| 1581 | E₁ₐE₂EᵢᵥE₄ | | | Eₑ |
| 1582 | E_{1b}E₂EᵢᵥE₄ | | | Eₑ |
| 1583 | E₁ₐE₂E₃EᵢᵥE₄ | | | Eₑ |
| 1584 | E_{1b}E₂E₃EᵢᵥE₄ | | | Eₑ |
| 1585 | E₁ₐE₂ | | E₄* | Eₑ |
| 1586 | E_{1b}E₂ | | E₄* | Eₑ |
| 1587 | E₁ₐE₂E₃ | | E₄* | Eₑ |
| 1588 | E_{1b}E₂E₃ | | E₄* | Eₑ |
| 1589 | E₁ₐE₂Eᵢ | | E₄* | Eₑ |
| 1590 | E_{1b}E₂Eᵢ | | E₄* | Eₑ |
| 1591 | E₁ₐE₂Eᵢᵢ | | E₄* | Eₑ |
| 1592 | E_{1b}E₂Eᵢᵢ | | E₄* | Eₑ |
| 1593 | E₁ₐE₂EᵢᵢE_{iib} | | E₄* | Eₑ |
| 1594 | E_{1b}E₂EᵢᵢE_{iib} | | E₄* | Eₑ |
| 1595 | E₁ₐE₂E₃Eᵢ | | E₄* | Eₑ |
| 1596 | E_{1b}E₂E₃Eᵢ | | E₄* | Eₑ |
| 1597 | E₁ₐE₂E₃Eᵢᵢ | | E₄* | Eₑ |
| 1598 | E_{1b}E₂E₃Eᵢᵢ | | E₄* | Eₑ |
| 1599 | E₁ₐE₂E₃EᵢᵢE_{iib} | | E₄* | Eₑ |
| 1600 | E_{1b}E₂E₃EᵢᵢE_{iib} | | E₄* | Eₑ |
| 1601 | E₁ₐE₂E₄ | | E₄* | Eₑ |
| 1602 | E_{1b}E₂E₄ | | E₄* | Eₑ |
| 1603 | E₁ₐE₂E₃E₄ | | E₄* | Eₑ |
| 1604 | E_{1b}E₂E₃E₄ | | E₄* | Eₑ |
| 1605 | E₁ₐE₂EᵢE₄ | | E₄* | Eₑ |
| 1606 | E_{1b}E₂EᵢE₄ | | E₄* | Eₑ |
| 1607 | E₁ₐE₂EᵢᵢE₄ | | E₄* | Eₑ |
| 1608 | E_{1b}E₂EᵢᵢE₄ | | E₄* | Eₑ |
| 1609 | E₁ₐE₂EᵢᵢE_{iib}E₄ | | E₄* | Eₑ |
| 1610 | E_{1b}E₂EᵢᵢE_{iib}E₄ | | E₄* | Eₑ |
| 1611 | E₁ₐE₂E₃EᵢE₄ | | E₄* | Eₑ |
| 1612 | E_{1b}E₂E₃EᵢE₄ | | E₄* | Eₑ |
| 1613 | E₁ₐE₂E₃EᵢᵢE₄ | | E₄* | Eₑ |
| 1614 | E_{1b}E₂E₃EᵢᵢE₄ | | E₄* | Eₑ |
| 1615 | E₁ₐE₂E₃EᵢᵢE_{iib}E₄ | | E₄* | Eₑ |
| 1616 | E_{1b}E₂E₃EᵢᵢE_{iib}E₄ | | E₄* | Eₑ |
| 1617 | E₁ₐE₂Eᵢᵢᵢ | | E₄* | Eₑ |
| 1618 | E_{1b}E₂Eᵢᵢᵢ | | E₄* | Eₑ |
| 1619 | E₁ₐE₂E₃Eᵢᵢᵢ | | E₄* | Eₑ |
| 1620 | E_{1b}E₂E₃Eᵢᵢᵢ | | E₄* | Eₑ |
| 1621 | E₁ₐE₂EᵢᵢᵢE₄ | | E₄* | Eₑ |
| 1622 | E_{1b}E₂EᵢᵢᵢE₄ | | E₄* | Eₑ |
| 1623 | E₁ₐE₂E₃EᵢᵢᵢE₄ | | E₄* | Eₑ |
| 1624 | E_{1b}E₂E₃EᵢᵢᵢE₄ | | E₄* | Eₑ |
| 1625 | E₁ₐE₂Eᵢᵥ | | E₄* | Eₑ |
| 1626 | E_{1b}E₂Eᵢᵥ | | E₄* | Eₑ |
| 1627 | E₁ₐE₂E₃Eᵢᵥ | | E₄* | Eₑ |
| 1628 | E_{1b}E₂E₃Eᵢᵥ | | E₄* | Eₑ |
| 1629 | E₁ₐE₂EᵢᵥE₄ | | E₄* | Eₑ |
| 1630 | E_{1b}E₂EᵢᵥE₄ | | E₄* | Eₑ |
| 1631 | E₁ₐE₂E₃EᵢᵥE₄ | | E₄* | Eₑ |
| 1632 | E_{1b}E₂E₃EᵢᵥE₄ | | E₄* | Eₑ |
| 1633 | E₁ₐE₂ | | E₅ | Eₑ |
| 1634 | E_{1b}E₂ | | E₅ | Eₑ |
| 1635 | E₁ₐE₂E₃ | | E₅ | Eₑ |
| 1636 | E_{1b}E₂E₃ | | E₅ | Eₑ |
| 1637 | E₁ₐE₂Eᵢ | | E₅ | Eₑ |
| 1638 | E_{1b}E₂Eᵢ | | E₅ | Eₑ |
| 1639 | E₁ₐE₂Eᵢᵢ | | E₅ | Eₑ |
| 1640 | E_{1b}E₂Eᵢᵢ | | E₅ | Eₑ |
| 1641 | E₁ₐE₂EᵢᵢE_{iib} | | E₅ | Eₑ |
| 1642 | E_{1b}E₂EᵢᵢE_{iib} | | E₅ | Eₑ |
| 1643 | E₁ₐE₂E₃Eᵢ | | E₅ | Eₑ |
| 1644 | E_{1b}E₂E₃Eᵢ | | E₅ | Eₑ |
| 1645 | E₁ₐE₂E₃Eᵢᵢ | | E₅ | Eₑ |
| 1646 | E_{1b}E₂E₃Eᵢᵢ | | E₅ | Eₑ |
| 1647 | E₁ₐE₂E₃EᵢᵢE_{iib} | | E₅ | Eₑ |
| 1648 | E_{1b}E₂E₃EᵢᵢE_{iib} | | E₅ | Eₑ |
| 1649 | E₁ₐE₂E₄ | | E₅ | Eₑ |
| 1650 | E_{1b}E₂E₄ | | E₅ | Eₑ |
| 1651 | E₁ₐE₂E₃E₄ | | E₅ | Eₑ |
| 1652 | E_{1b}E₂E₃E₄ | | E₅ | Eₑ |
| 1653 | E₁ₐE₂EᵢE₄ | | E₅ | Eₑ |
| 1654 | E_{1b}E₂EᵢE₄ | | E₅ | Eₑ |
| 1655 | E₁ₐE₂EᵢᵢE₄ | | E₅ | Eₑ |
| 1656 | E_{1b}E₂EᵢᵢE₄ | | E₅ | Eₑ |
| 1657 | E₁ₐE₂EᵢᵢE_{iib}E₄ | | E₅ | Eₑ |
| 1658 | E_{1b}E₂EᵢᵢE_{iib}E₄ | | E₅ | Eₑ |
| 1659 | E₁ₐE₂E₃EᵢE₄ | | E₅ | Eₑ |
| 1660 | E_{1b}E₂E₃EᵢE₄ | | E₅ | Eₑ |
| 1661 | E₁ₐE₂E₃EᵢᵢE₄ | | E₅ | Eₑ |
| 1662 | E_{1b}E₂E₃EᵢᵢE₄ | | E₅ | Eₑ |
| 1663 | E₁ₐE₂E₃EᵢᵢE_{iib}E₄ | | E₅ | Eₑ |
| 1664 | E_{1b}E₂E₃EᵢᵢE_{iib}E₄ | | E₅ | Eₑ |
| 1665 | E₁ₐE₂Eᵢᵢᵢ | | E₅ | Eₑ |
| 1666 | E_{1b}E₂Eᵢᵢᵢ | | E₅ | Eₑ |
| 1667 | E₁ₐE₂E₃Eᵢᵢᵢ | | E₅ | Eₑ |
| 1668 | E_{1b}E₂E₃Eᵢᵢᵢ | | E₅ | Eₑ |
| 1669 | E₁ₐE₂EᵢᵢᵢE₄ | | E₅ | Eₑ |
| 1670 | E_{1b}E₂EᵢᵢᵢE₄ | | E₅ | Eₑ |
| 1671 | E₁ₐE₂E₃EᵢᵢᵢE₄ | | E₅ | Eₑ |
| 1672 | E_{1b}E₂E₃EᵢᵢᵢE₄ | | E₅ | Eₑ |
| 1673 | E₁ₐE₂Eᵢᵥ | | E₅ | Eₑ |
| 1674 | E_{1b}E₂Eᵢᵥ | | E₅ | Eₑ |
| 1675 | E₁ₐE₂E₃Eᵢᵥ | | E₅ | Eₑ |
| 1676 | E_{1b}E₂E₃Eᵢᵥ | | E₅ | Eₑ |
| 1677 | E₁ₐE₂EᵢᵥE₄ | | E₅ | Eₑ |
| 1678 | E_{1b}E₂EᵢᵥE₄ | | E₅ | Eₑ |
| 1679 | E₁ₐE₂E₃EᵢᵥE₄ | | E₅ | Eₑ |
| 1680 | E_{1b}E₂E₃EᵢᵥE₄ | | E₅ | Eₑ |
| 1681 | E₁ₐE₂ | | E₄*E₅ | Eₑ |
| 1682 | E_{1b}E₂ | | E₄*E₅ | Eₑ |
| 1683 | E₁ₐE₂E₃ | | E₄*E₅ | Eₑ |
| 1684 | E_{1b}E₂E₃ | | E₄*E₅ | Eₑ |
| 1685 | E₁ₐE₂Eᵢ | | E₄*E₅ | Eₑ |
| 1686 | E_{1b}E₂Eᵢ | | E₄*E₅ | Eₑ |
| 1687 | E₁ₐE₂Eᵢᵢ | | E₄*E₅ | Eₑ |
| 1688 | E_{1b}E₂Eᵢᵢ | | E₄*E₅ | Eₑ |
| 1689 | E₁ₐE₂EᵢᵢE_{iib} | | E₄*E₅ | Eₑ |
| 1690 | E_{1b}E₂EᵢᵢE_{iib} | | E₄*E₅ | Eₑ |
| 1691 | E₁ₐE₂E₃Eᵢ | | E₄*E₅ | Eₑ |
| 1692 | E_{1b}E₂E₃Eᵢ | | E₄*E₅ | Eₑ |
| 1693 | E₁ₐE₂E₃Eᵢᵢ | | E₄*E₅ | Eₑ |
| 1694 | E_{1b}E₂E₃Eᵢᵢ | | E₄*E₅ | Eₑ |
| 1695 | E₁ₐE₂E₃EᵢᵢE_{iib} | | E₄*E₅ | Eₑ |
| 1696 | E_{1b}E₂E₃EᵢᵢE_{iib} | | E₄*E₅ | Eₑ |
| 1697 | E₁ₐE₂E₄ | | E₄*E₅ | Eₑ |
| 1698 | E_{1b}E₂E₄ | | E₄*E₅ | Eₑ |
| 1699 | E₁ₐE₂E₃E₄ | | E₄*E₅ | Eₑ |
| 1700 | E_{1b}E₂E₃E₄ | | E₄*E₅ | Eₑ |
| 1701 | E₁ₐE₂EᵢE₄ | | E₄*E₅ | Eₑ |
| 1702 | E_{1b}E₂EᵢE₄ | | E₄*E₅ | Eₑ |
| 1703 | E₁ₐE₂EᵢᵢE₄ | | E₄*E₅ | Eₑ |
| 1704 | E_{1b}E₂EᵢᵢE₄ | | E₄*E₅ | Eₑ |
| 1705 | E₁ₐE₂EᵢᵢE_{iib}E₄ | | E₄*E₅ | Eₑ |
| 1706 | E_{1b}E₂EᵢᵢE_{iib}E₄ | | E₄*E₅ | Eₑ |
| 1707 | E₁ₐE₂E₃EᵢE₄ | | E₄*E₅ | Eₑ |
| 1708 | E_{1b}E₂E₃EᵢE₄ | | E₄*E₅ | Eₑ |
| 1709 | E₁ₐE₂E₃EᵢᵢE₄ | | E₄*E₅ | Eₑ |
| 1710 | E_{1b}E₂E₃EᵢᵢE₄ | | E₄*E₅ | Eₑ |
| 1711 | E₁ₐE₂E₃EᵢᵢE_{iib}E₄ | | E₄*E₅ | Eₑ |
| 1712 | E_{1b}E₂E₃EᵢᵢE_{iib}E₄ | | E₄*E₅ | Eₑ |
| 1713 | E₁ₐE₂Eᵢᵢᵢ | | E₄*E₅ | Eₑ |
| 1714 | E_{1b}E₂Eᵢᵢᵢ | | E₄*E₅ | Eₑ |
| 1715 | E₁ₐE₂E₃Eᵢᵢᵢ | | E₄*E₅ | Eₑ |
| 1716 | E_{1b}E₂E₃Eᵢᵢᵢ | | E₄*E₅ | Eₑ |
| 1717 | E₁ₐE₂EᵢᵢᵢE₄ | | E₄*E₅ | Eₑ |
| 1718 | E_{1b}E₂EᵢᵢᵢE₄ | | E₄*E₅ | Eₑ |
| 1719 | E₁ₐE₂E₃EᵢᵢᵢE₄ | | E₄*E₅ | Eₑ |
| 1720 | E_{1b}E₂E₃EᵢᵢᵢE₄ | | E₄*E₅ | Eₑ |
| 1721 | E₁ₐE₂Eᵢᵥ | | E₄*E₅ | Eₑ |
| 1722 | E_{1b}E₂Eᵢᵥ | | E₄*E₅ | Eₑ |
| 1723 | E₁ₐE₂E₃Eᵢᵥ | | E₄*E₅ | Eₑ |
| 1724 | E_{1b}E₂E₃Eᵢᵥ | | E₄*E₅ | Eₑ |
| 1725 | E₁ₐE₂EᵢᵥE₄ | | E₄*E₅ | Eₑ |
| 1726 | E_{1b}E₂EᵢᵥE₄ | | E₄*E₅ | Eₑ |
| 1727 | E₁ₐE₂E₃EᵢᵥE₄ | | E₄*E₅ | Eₑ |
| 1728 | E_{1b}E₂E₃EᵢᵥE₄ | | E₄*E₅ | Eₑ |
| 1729 | E₁ₐE₂ | AlkL | | Eₑ |
| 1730 | E_{1b}E₂ | AlkL | | Eₑ |
| 1731 | E₁ₐE₂E₃ | AlkL | | Eₑ |
| 1732 | E_{1b}E₂E₃ | AlkL | | Eₑ |
| 1733 | E₁ₐE₂Eᵢ | AlkL | | Eₑ |
| 1734 | E_{1b}E₂Eᵢ | AlkL | | Eₑ |
| 1735 | E₁ₐE₂Eᵢᵢ | AlkL | | Eₑ |
| 1736 | E_{1b}E₂Eᵢᵢ | AlkL | | Eₑ |
| 1737 | E₁ₐE₂EᵢᵢE_{iib} | AlkL | | Eₑ |
| 1738 | E_{1b}E₂EᵢᵢE_{iib} | AlkL | | Eₑ |
| 1739 | E₁ₐE₂E₃Eᵢ | AlkL | | Eₑ |
| 1740 | E_{1b}E₂E₃Eᵢ | AlkL | | Eₑ |
| 1741 | E₁ₐE₂E₃Eᵢᵢ | AlkL | | Eₑ |
| 1742 | E_{1b}E₂E₃Eᵢᵢ | AlkL | | Eₑ |
| 1743 | E₁ₐE₂E₃EᵢᵢE_{iib} | AlkL | | Eₑ |
| 1744 | E_{1b}E₂E₃EᵢᵢE_{iib} | AlkL | | Eₑ |
| 1745 | E₁ₐE₂E₄ | AlkL | | Eₑ |
| 1746 | E_{1b}E₂E₄ | AlkL | | Eₑ |
| 1747 | E₁ₐE₂E₃E₄ | AlkL | | Eₑ |
| 1748 | E_{1b}E₂E₃E₄ | AlkL | | Eₑ |
| 1749 | E₁ₐE₂EᵢE₄ | AlkL | | Eₑ |
| 1750 | E_{1b}E₂EᵢE₄ | AlkL | | Eₑ |
| 1751 | E₁ₐE₂EᵢᵢE₄ | AlkL | | Eₑ |
| 1752 | E_{1b}E₂EᵢᵢE₄ | AlkL | | Eₑ |
| 1753 | E₁ₐE₂EᵢᵢE_{iib}E₄ | AlkL | | Eₑ |
| 1754 | E_{1b}E₂EᵢᵢE_{iib}E₄ | AlkL | | Eₑ |
| 1755 | E₁ₐE₂E₃EᵢE₄ | AlkL | | Eₑ |
| 1756 | E_{1b}E₂E₃EᵢE₄ | AlkL | | Eₑ |
| 1757 | E₁ₐE₂E₃EᵢᵢE₄ | AlkL | | Eₑ |
| 1758 | E_{1b}E₂E₃EᵢᵢE₄ | AlkL | | Eₑ |
| 1759 | E₁ₐE₂E₃EᵢᵢE_{iib}E₄ | AlkL | | Eₑ |
| 1760 | E_{1b}E₂E₃EᵢᵢE_{iib}E₄ | AlkL | | Eₑ |
| 1761 | E₁ₐE₂Eᵢᵢᵢ | AlkL | | Eₑ |
| 1762 | E_{1b}E₂Eᵢᵢᵢ | AlkL | | Eₑ |
| 1763 | E₁ₐE₂E₃Eᵢᵢᵢ | AlkL | | Eₑ |
| 1764 | E_{1b}E₂E₃Eᵢᵢᵢ | AlkL | | Eₑ |
| 1765 | E₁ₐE₂EᵢᵢᵢE₄ | AlkL | | Eₑ |
| 1766 | E_{1b}E₂EᵢᵢᵢE₄ | AlkL | | Eₑ |
| 1767 | E₁ₐE₂E₃EᵢᵢᵢE₄ | AlkL | | Eₑ |
| 1768 | E_{1b}E₂E₃EᵢᵢᵢE₄ | AlkL | | Eₑ |
| 1769 | E₁ₐE₂Eᵢᵥ | AlkL | | Eₑ |
| 1770 | E_{1b}E₂Eᵢᵥ | AlkL | | Eₑ |
| 1771 | E₁ₐE₂E₃Eᵢᵥ | AlkL | | Eₑ |
| 1772 | E_{1b}E₂E₃Eᵢᵥ | AlkL | | Eₑ |
| 1773 | E₁ₐE₂EᵢᵥE₄ | AlkL | | Eₑ |
| 1774 | E_{1b}E₂EᵢᵥE₄ | AlkL | | Eₑ |
| 1775 | E₁ₐE₂E₃EᵢᵥE₄ | AlkL | | Eₑ |
| 1776 | E_{1b}E₂E₃EᵢᵥE₄ | AlkL | | Eₑ |
| 1777 | E₁ₐE₂ | AlkL | E₄* | Eₑ |
| 1778 | E_{1b}E₂ | AlkL | E₄* | Eₑ |
| 1779 | E₁ₐE₂E₃ | AlkL | E₄* | Eₑ |
| 1780 | E_{1b}E₂E₃ | AlkL | E₄* | Eₑ |
| 1781 | E₁ₐE₂Eᵢ | AlkL | E₄* | Eₑ |
| 1782 | E_{1b}E₂Eᵢ | AlkL | E₄* | Eₑ |
| 1783 | E₁ₐE₂Eᵢᵢ | AlkL | E₄* | Eₑ |
| 1784 | E_{1b}E₂Eᵢᵢ | AlkL | E₄* | Eₑ |
| 1785 | E₁ₐE₂EᵢᵢE_{iib} | AlkL | E₄* | Eₑ |
| 1786 | E_{1b}E₂EᵢᵢE_{iib} | AlkL | E₄* | Eₑ |
| 1787 | E₁ₐE₂E₃Eᵢ | AlkL | E₄* | Eₑ |
| 1788 | E_{1b}E₂E₃Eᵢ | AlkL | E₄* | Eₑ |
| 1789 | E₁ₐE₂E₃Eᵢᵢ | AlkL | E₄* | Eₑ |
| 1790 | E_{1b}E₂E₃Eᵢᵢ | AlkL | E₄* | Eₑ |
| 1791 | E₁ₐE₂E₃EᵢᵢE_{iib} | AlkL | E₄* | Eₑ |
| 1792 | E_{1b}E₂E₃EᵢᵢE_{iib} | AlkL | E₄* | Eₑ |
| 1793 | E₁ₐE₂E₄ | AlkL | E₄* | Eₑ |
| 1794 | E_{1b}E₂E₄ | AlkL | E₄* | Eₑ |
| 1795 | E₁ₐE₂E₃E₄ | AlkL | E₄* | Eₑ |
| 1796 | E_{1b}E₂E₃E₄ | AlkL | E₄* | Eₑ |
| 1797 | E₁ₐE₂EᵢE₄ | AlkL | E₄* | Eₑ |
| 1798 | E_{1b}E₂EᵢE₄ | AlkL | E₄* | Eₑ |
| 1799 | E₁ₐE₂EᵢᵢE₄ | AlkL | E₄* | Eₑ |
| 1800 | E_{1b}E₂EᵢᵢE₄ | AlkL | E₄* | Eₑ |
| 1801 | E₁ₐE₂EᵢᵢE_{iib}E₄ | AlkL | E₄* | Eₑ |
| 1802 | E_{1b}E₂EᵢᵢE_{iib}E₄ | AlkL | E₄* | Eₑ |
| 1803 | E₁ₐE₂E₃EᵢE₄ | AlkL | E₄* | Eₑ |
| 1804 | E_{1b}E₂E₃EᵢE₄ | AlkL | E₄* | Eₑ |
| 1805 | E₁ₐE₂E₃EᵢᵢE₄ | AlkL | E₄* | Eₑ |
| 1806 | E_{1b}E₂E₃EᵢᵢE₄ | AlkL | E₄* | Eₑ |
| 1807 | E₁ₐE₂E₃EᵢᵢE_{iib}E₄ | AlkL | E₄* | Eₑ |
| 1808 | E_{1b}E₂E₃EᵢᵢE_{iib}E₄ | AlkL | E₄* | Eₑ |
| 1809 | E₁ₐE₂Eᵢᵢᵢ | AlkL | E₄* | Eₑ |
| 1810 | E_{1b}E₂Eᵢᵢᵢ | AlkL | E₄* | Eₑ |
| 1811 | E₁ₐE₂E₃Eᵢᵢᵢ | AlkL | E₄* | Eₑ |
| 1812 | E_{1b}E₂E₃Eᵢᵢᵢ | AlkL | E₄* | Eₑ |
| 1813 | E₁ₐE₂EᵢᵢᵢE₄ | AlkL | E₄* | Eₑ |
| 1814 | E_{1b}E₂EᵢᵢᵢE₄ | AlkL | E₄* | Eₑ |
| 1815 | E₁ₐE₂E₃EᵢᵢᵢE₄ | AlkL | E₄* | Eₑ |
| 1816 | E_{1b}E₂E₃EᵢᵢᵢE₄ | AlkL | E₄* | Eₑ |
| 1817 | E₁ₐE₂Eᵢᵥ | AlkL | E₄* | Eₑ |
| 1818 | E_{1b}E₂Eᵢᵥ | AlkL | E₄* | Eₑ |
| 1819 | E₁ₐE₂E₃Eᵢᵥ | AlkL | E₄* | Eₑ |
| 1820 | E_{1b}E₂E₃Eᵢᵥ | AlkL | E₄* | Eₑ |
| 1821 | E₁ₐE₂EᵢᵥE₄ | AlkL | E₄* | Eₑ |
| 1822 | E_{1b}E₂EᵢᵥE₄ | AlkL | E₄* | Eₑ |
| 1823 | E₁ₐE₂E₃EᵢᵥE₄ | AlkL | E₄* | Eₑ |
| 1824 | E_{1b}E₂E₃EᵢᵥE₄ | AlkL | E₄* | Eₑ |
| 1825 | E₁ₐE₂ | AlkL | E₅ | Eₑ |
| 1826 | E_{1b}E₂ | AlkL | E₅ | Eₑ |
| 1827 | E₁ₐE₂E₃ | AlkL | E₅ | Eₑ |
| 1828 | E_{1b}E₂E₃ | AlkL | E₅ | Eₑ |
| 1829 | E₁ₐE₂Eᵢ | AlkL | E₅ | Eₑ |
| 1830 | E_{1b}E₂Eᵢ | AlkL | E₅ | Eₑ |
| 1831 | E₁ₐE₂Eᵢᵢ | AlkL | E₅ | Eₑ |
| 1832 | E_{1b}E₂Eᵢᵢ | AlkL | E₅ | Eₑ |
| 1833 | E₁ₐE₂EᵢᵢE_{iib} | AlkL | E₅ | Eₑ |
| 1834 | E_{1b}E₂EᵢᵢE_{iib} | AlkL | E₅ | Eₑ |
| 1835 | E₁ₐE₂E₃Eᵢ | AlkL | E₅ | Eₑ |
| 1836 | E_{1b}E₂E₃Eᵢ | AlkL | E₅ | Eₑ |
| 1837 | E₁ₐE₂E₃Eᵢᵢ | AlkL | E₅ | Eₑ |
| 1838 | E_{1b}E₂E₃Eᵢᵢ | AlkL | E₅ | Eₑ |
| 1839 | E₁ₐE₂E₃EᵢᵢE_{iib} | AlkL | E₅ | Eₑ |
| 1840 | E_{1b}E₂E₃EᵢᵢE_{iib} | AlkL | E₅ | Eₑ |
| 1841 | E₁ₐE₂E₄ | AlkL | E₅ | Eₑ |
| 1842 | E_{1b}E₂E₄ | AlkL | E₅ | Eₑ |
| 1843 | E₁ₐE₂E₃E₄ | AlkL | E₅ | Eₑ |
| 1844 | E_{1b}E₂E₃E₄ | AlkL | E₅ | Eₑ |
| 1845 | E₁ₐE₂EᵢE₄ | AlkL | E₅ | Eₑ |
| 1846 | E_{1b}E₂EᵢE₄ | AlkL | E₅ | Eₑ |
| 1847 | E₁ₐE₂EᵢᵢE₄ | AlkL | E₅ | Eₑ |
| 1848 | E_{1b}E₂EᵢᵢE₄ | AlkL | E₅ | Eₑ |
| 1849 | E₁ₐE₂EᵢᵢE_{iib}E₄ | AlkL | E₅ | Eₑ |
| 1850 | E_{1b}E₂EᵢᵢE_{iib}E₄ | AlkL | E₅ | Eₑ |
| 1851 | E₁ₐE₂E₃EᵢE₄ | AlkL | E₅ | Eₑ |
| 1852 | E_{1b}E₂E₃EᵢE₄ | AlkL | E₅ | Eₑ |
| 1853 | E₁ₐE₂E₃EᵢᵢE₄ | AlkL | E₅ | Eₑ |
| 1854 | E_{1b}E₂E₃EᵢᵢE₄ | AlkL | E₅ | Eₑ |
| 1855 | E₁ₐE₂E₃EᵢᵢE_{iib}E₄ | AlkL | E₅ | Eₑ |
| 1856 | E_{1b}E₂E₃EᵢᵢE_{iib}E₄ | AlkL | E₅ | Eₑ |
| 1857 | E₁ₐE₂Eᵢᵢᵢ | AlkL | E₅ | Eₑ |
| 1858 | E_{1b}E₂Eᵢᵢᵢ | AlkL | E₅ | Eₑ |
| 1859 | E₁ₐE₂E₃Eᵢᵢᵢ | AlkL | E₅ | Eₑ |
| 1860 | E_{1b}E₂E₃Eᵢᵢᵢ | AlkL | E₅ | Eₑ |
| 1861 | E₁ₐE₂EᵢᵢᵢE₄ | AlkL | E₅ | Eₑ |
| 1862 | E_{1b}E₂EᵢᵢᵢE₄ | AlkL | E₅ | Eₑ |
| 1863 | E₁ₐE₂E₃EᵢᵢᵢE₄ | AlkL | E₅ | Eₑ |
| 1864 | E_{1b}E₂E₃EᵢᵢᵢE₄ | AlkL | E₅ | Eₑ |
| 1865 | E₁ₐE₂Eᵢᵥ | AlkL | E₅ | Eₑ |
| 1866 | E_{1b}E₂Eᵢᵥ | AlkL | E₅ | Eₑ |
| 1867 | E₁ₐE₂E₃Eᵢᵥ | AlkL | E₅ | Eₑ |
| 1868 | E_{1b}E₂E₃Eᵢᵥ | AlkL | E₅ | Eₑ |
| 1869 | E₁ₐE₂EᵢᵥE₄ | AlkL | E₅ | Eₑ |
| 1870 | E_{1b}E₂EᵢᵥE₄ | AlkL | E₅ | Eₑ |
| 1871 | E₁ₐE₂E₃EᵢᵥE₄ | AlkL | E₅ | Eₑ |
| 1872 | E_{1b}E₂E₃EᵢᵥE₄ | AlkL | E₅ | Eₑ |
| 1873 | E₁ₐE₂ | AlkL | E₄*E₅ | Eₑ |
| 1874 | E_{1b}E₂ | AlkL | E₄*E₅ | Eₑ |
| 1875 | E₁ₐE₂E₃ | AlkL | E₄*E₅ | Eₑ |
| 1876 | E_{1b}E₂E₃ | AlkL | E₄*E₅ | Eₑ |
| 1877 | E₁ₐE₂Eᵢ | AlkL | E₄*E₅ | Eₑ |
| 1878 | E_{1b}E₂Eᵢ | AlkL | E₄*E₅ | Eₑ |
| 1879 | E₁ₐE₂Eᵢᵢ | AlkL | E₄*E₅ | Eₑ |
| 1880 | E_{1b}E₂Eᵢᵢ | AlkL | E₄*E₅ | Eₑ |
| 1881 | E₁ₐE₂EᵢᵢE_{iib} | AlkL | E₄*E₅ | Eₑ |
| 1882 | E_{1b}E₂EᵢᵢE_{iib} | AlkL | E₄*E₅ | Eₑ |
| 1883 | E₁ₐE₂E₃Eᵢ | AlkL | E₄*E₅ | Eₑ |
| 1884 | E_{1b}E₂E₃Eᵢ | AlkL | E₄*E₅ | Eₑ |
| 1885 | E₁ₐE₂E₃Eᵢᵢ | AlkL | E₄*E₅ | Eₑ |
| 1886 | E_{1b}E₂E₃Eᵢᵢ | AlkL | E₄*E₅ | Eₑ |
| 1887 | E₁ₐE₂E₃EᵢᵢE_{iib} | AlkL | E₄*E₅ | Eₑ |
| 1888 | E_{1b}E₂E₃EᵢᵢE_{iib} | AlkL | E₄*E₅ | Eₑ |
| 1889 | E₁ₐE₂E₄ | AlkL | E₄*E₅ | Eₑ |
| 1890 | E_{1b}E₂E₄ | AlkL | E₄*E₅ | Eₑ |
| 1891 | E₁ₐE₂E₃E₄ | AlkL | E₄*E₅ | Eₑ |
| 1892 | E_{1b}E₂E₃E₄ | AlkL | E₄*E₅ | Eₑ |
| 1893 | E₁ₐE₂EᵢE₄ | AlkL | E₄*E₅ | Eₑ |
| 1894 | E_{1b}E₂EᵢE₄ | AlkL | E₄*E₅ | Eₑ |
| 1895 | E₁ₐE₂EᵢᵢE₄ | AlkL | E₄*E₅ | Eₑ |
| 1896 | E_{1b}E₂EᵢᵢE₄ | AlkL | E₄*E₅ | Eₑ |
| 1897 | E₁ₐE₂EᵢᵢE_{iib}E₄ | AlkL | E₄*E₅ | Eₑ |
| 1898 | E_{1b}E₂EᵢᵢE_{iib}E₄ | AlkL | E₄*E₅ | Eₑ |
| 1899 | E₁ₐE₂E₃EᵢE₄ | AlkL | E₄*E₅ | Eₑ |
| 1900 | E_{1b}E₂E₃EᵢE₄ | AlkL | E₄*E₅ | Eₑ |
| 1901 | E₁ₐE₂E₃EᵢᵢE₄ | AlkL | E₄*E₅ | Eₑ |
| 1902 | E_{1b}E₂E₃EᵢᵢE₄ | AlkL | E₄*E₅ | Eₑ |
| 1903 | E₁ₐE₂E₃EᵢᵢE_{iib}E₄ | AlkL | E₄*E₅ | Eₑ |
| 1904 | E_{1b}E₂E₃EᵢᵢE_{iib}E₄ | AlkL | E₄*E₅ | Eₑ |
| 1905 | E₁ₐE₂Eᵢᵢᵢ | AlkL | E₄*E₅ | Eₑ |
| 1906 | E_{1b}E₂Eᵢᵢᵢ | AlkL | E₄*E₅ | Eₑ |
| 1907 | E₁ₐE₂E₃Eᵢᵢᵢ | AlkL | E₄*E₅ | Eₑ |
| 1908 | E_{1b}E₂E₃Eᵢᵢᵢ | AlkL | E₄*E₅ | Eₑ |
| 1909 | E₁ₐE₂EᵢᵢᵢE₄ | AlkL | E₄*E₅ | Eₑ |
| 1910 | E_{1b}E₂EᵢᵢᵢE₄ | AlkL | E₄*E₅ | Eₑ |
| 1911 | E₁ₐE₂E₃EᵢᵢᵢE₄ | AlkL | E₄*E₅ | Eₑ |
| 1912 | E_{1b}E₂E₃EᵢᵢᵢE₄ | AlkL | E₄*E₅ | Eₑ |
| 1913 | E₁ₐE₂Eᵢᵥ | AlkL | E₄*E₅ | Eₑ |
| 1914 | E_{1b}E₂Eᵢᵥ | AlkL | E₄*E₅ | Eₑ |
| 1915 | E₁ₐE₂E₃Eᵢᵥ | AlkL | E₄*E₅ | Eₑ |
| 1916 | E_{1b}E₂E₃Eᵢᵥ | AlkL | E₄*E₅ | Eₑ |
| 1917 | E₁ₐE₂EᵢᵥE₄ | AlkL | E₄*E₅ | Eₑ |
| 1918 | E_{1b}E₂EᵢᵥE₄ | AlkL | E₄*E₅ | Eₑ |
| 1919 | E₁ₐE₂E₃EᵢᵥE₄ | AlkL | E₄*E₅ | Eₑ |
| 1920 | E_{1b}E₂E₃EᵢᵥE₄ | AlkL | E₄*E₅ | Eₑ |
| 1921 | E₁ₐE₂ | | | E_{f} |
| 1922 | E_{1b}E₂ | | | E_{f} |
| 1923 | E₁ₐE₂E₃ | | | E_{f} |
| 1924 | E_{1b}E₂E₃ | | | E_{f} |
| 1925 | E₁ₐE₂Eᵢ | | | E_{f} |
| 1926 | E_{1b}E₂Eᵢ | | | E_{f} |
| 1927 | E₁ₐE₂Eᵢᵢ | | | E_{f} |
| 1928 | E_{1b}E₂Eᵢᵢ | | | E_{f} |
| 1929 | E₁ₐE₂EᵢᵢE_{iib} | | | E_{f} |
| 1930 | E_{1b}E₂EᵢᵢE_{iib} | | | E_{f} |
| 1931 | E₁ₐE₂E₃Eᵢ | | | E_{f} |
| 1932 | E_{1b}E₂E₃Eᵢ | | | E_{f} |
| 1933 | E₁ₐE₂E₃Eᵢᵢ | | | E_{f} |
| 1934 | E_{1b}E₂E₃Eᵢᵢ | | | E_{f} |
| 1935 | E₁ₐE₂E₃EᵢᵢE_{iib} | | | E_{f} |
| 1936 | E_{1b}E₂E₃EᵢᵢE_{iib} | | | E_{f} |
| 1937 | E₁ₐE₂E₄ | | | E_{f} |
| 1938 | E_{1b}E₂E₄ | | | E_{f} |
| 1939 | E₁ₐE₂E₃E₄ | | | E_{f} |
| 1940 | E_{1b}E₂E₃E₄ | | | E_{f} |
| 1941 | E₁ₐE₂EᵢE₄ | | | E_{f} |
| 1942 | E_{1b}E₂EᵢE₄ | | | E_{f} |
| 1943 | E₁ₐE₂EᵢᵢE₄ | | | E_{f} |
| 1944 | E_{1b}E₂EᵢᵢE₄ | | | E_{f} |
| 1945 | E₁ₐE₂EᵢᵢE_{iib}E₄ | | | E_{f} |
| 1946 | E_{1b}E₂EᵢᵢE_{iib}E₄ | | | E_{f} |
| 1947 | E₁ₐE₂E₃EᵢE₄ | | | E_{f} |
| 1948 | E_{1b}E₂E₃EᵢE₄ | | | E_{f} |
| 1949 | E₁ₐE₂E₃EᵢᵢE₄ | | | E_{f} |
| 1950 | E_{1b}E₂E₃EᵢᵢE₄ | | | E_{f} |
| 1951 | E₁ₐE₂E₃EᵢᵢE_{iib}E₄ | | | E_{f} |
| 1952 | E_{1b}E₂E₃EᵢᵢE_{iib}E₄ | | | E_{f} |
| 1953 | E₁ₐE₂Eᵢᵢᵢ | | | E_{f} |
| 1954 | E_{1b}E₂Eᵢᵢᵢ | | | E_{f} |
| 1955 | E₁ₐE₂E₃Eᵢᵢᵢ | | | E_{f} |
| 1956 | E_{1b}E₂E₃Eᵢᵢᵢ | | | E_{f} |
| 1957 | E₁ₐE₂EᵢᵢᵢE₄ | | | E_{f} |
| 1958 | E_{1b}E₂EᵢᵢᵢE₄ | | | E_{f} |
| 1959 | E₁ₐE₂E₃EᵢᵢᵢE₄ | | | E_{f} |
| 1960 | E_{1b}E₂E₃EᵢᵢᵢE₄ | | | E_{f} |
| 1961 | E₁ₐE₂Eᵢᵥ | | | E_{f} |
| 1962 | E_{1b}E₂Eᵢᵥ | | | E_{f} |
| 1963 | E₁ₐE₂E₃Eᵢᵥ | | | E_{f} |
| 1964 | E_{1b}E₂E₃Eᵢᵥ | | | E_{f} |
| 1965 | E₁ₐE₂EᵢᵥE₄ | | | E_{f} |
| 1966 | E_{1b}E₂EᵢᵥE₄ | | | E_{f} |
| 1967 | E₁ₐE₂E₃EᵢᵥE₄ | | | E_{f} |
| 1968 | E_{1b}E₂E₃EᵢᵥE₄ | | | E_{f} |
| 1969 | E₁ₐE₂ | | E₄* | E_{f} |
| 1970 | E_{1b}E₂ | | E₄* | E_{f} |
| 1971 | E₁ₐE₂E₃ | | E₄* | E_{f} |
| 1972 | E_{1b}E₂E₃ | | E₄* | E_{f} |
| 1973 | E₁ₐE₂Eᵢ | | E₄* | E_{f} |
| 1974 | E_{1b}E₂Eᵢ | | E₄* | E_{f} |
| 1975 | E₁ₐE₂Eᵢᵢ | | E₄* | E_{f} |
| 1976 | E_{1b}E₂Eᵢᵢ | | E₄* | E_{f} |
| 1977 | E₁ₐE₂EᵢᵢE_{iib} | | E₄* | E_{f} |
| 1978 | E_{1b}E₂EᵢᵢE_{iib} | | E₄* | E_{f} |
| 1979 | E₁ₐE₂E₃Eᵢ | | E₄* | E_{f} |
| 1980 | E_{1b}E₂E₃Eᵢ | | E₄* | E_{f} |
| 1981 | E₁ₐE₂E₃Eᵢᵢ | | E₄* | E_{f} |
| 1982 | E_{1b}E₂E₃Eᵢᵢ | | E₄* | E_{f} |
| 1983 | E₁ₐE₂E₃EᵢᵢE_{iib} | | E₄* | E_{f} |
| 1984 | E_{1b}E₂E₃EᵢᵢE_{iib} | | E₄* | E_{f} |
| 1985 | E₁ₐE₂E₄ | | E₄* | E_{f} |
| 1986 | E_{1b}E₂E₄ | | E₄* | E_{f} |
| 1987 | E₁ₐE₂E₃E₄ | | E₄* | E_{f} |
| 1988 | E_{1b}E₂E₃E₄ | | E₄* | E_{f} |
| 1989 | E₁ₐE₂EᵢE₄ | | E₄* | E_{f} |
| 1990 | E_{1b}E₂EᵢE₄ | | E₄* | E_{f} |
| 1991 | E₁ₐE₂EᵢᵢE₄ | | E₄* | E_{f} |
| 1992 | E_{1b}E₂EᵢᵢE₄ | | E₄* | E_{f} |
| 1993 | E₁ₐE₂EᵢᵢE_{iib}E₄ | | E₄* | E_{f} |
| 1994 | E_{1b}E₂EᵢᵢE_{iib}E₄ | | E₄* | E_{f} |
| 1995 | E₁ₐE₂E₃EᵢE₄ | | E₄* | E_{f} |
| 1996 | E_{1b}E₂E₃EᵢE₄ | | E₄* | E_{f} |
| 1997 | E₁ₐE₂E₃EᵢᵢE₄ | | E₄* | E_{f} |
| 1998 | E_{1b}E₂E₃EᵢᵢE₄ | | E₄* | E_{f} |
| 1999 | E₁ₐE₂E₃EᵢᵢE_{iib}E₄ | | E₄* | E_{f} |
| 2000 | E_{1b}E₂E₃EᵢᵢE_{iib}E₄ | | E₄* | E_{f} |
| 2001 | E₁ₐE₂Eᵢᵢᵢ | | E₄* | E_{f} |
| 2002 | E_{1b}E₂Eᵢᵢᵢ | | E₄* | E_{f} |
| 2003 | E₁ₐE₂E₃Eᵢᵢᵢ | | E₄* | E_{f} |
| 2004 | E_{1b}E₂E₃Eᵢᵢᵢ | | E₄* | E_{f} |
| 2005 | E₁ₐE₂EᵢᵢᵢE₄ | | E₄* | E_{f} |
| 2006 | E_{1b}E₂EᵢᵢᵢE₄ | | E₄* | E_{f} |
| 2007 | E₁ₐE₂E₃EᵢᵢᵢE₄ | | E₄* | E_{f} |
| 2008 | E_{1b}E₂E₃EᵢᵢᵢE₄ | | E₄* | E_{f} |
| 2009 | E₁ₐE₂Eᵢᵥ | | E₄* | E_{f} |
| 2010 | E_{1b}E₂Eᵢᵥ | | E₄* | E_{f} |
| 2011 | E₁ₐE₂E₃Eᵢᵥ | | E₄* | E_{f} |
| 2012 | E_{1b}E₂E₃Eᵢᵥ | | E₄* | E_{f} |
| 2013 | E₁ₐE₂EᵢᵥE₄ | | E₄* | E_{f} |
| 2014 | E_{1b}E₂EᵢᵥE₄ | | E₄* | E_{f} |
| 2015 | E₁ₐE₂E₃EᵢᵥE₄ | | E₄* | E_{f} |
| 2016 | E_{1b}E₂E₃EᵢᵥE₄ | | E₄* | E_{f} |
| 2017 | E₁ₐE₂ | | E₅ | E_{f} |
| 2018 | E_{1b}E₂ | | E₅ | E_{f} |
| 2019 | E₁ₐE₂E₃ | | E₅ | E_{f} |
| 2020 | E_{1b}E₂E₃ | | E₅ | E_{f} |
| 2021 | E₁ₐE₂Eᵢ | | E₅ | E_{f} |
| 2022 | E_{1b}E₂Eᵢ | | E₅ | E_{f} |
| 2023 | E₁ₐE₂Eᵢᵢ | | E₅ | E_{f} |
| 2024 | E_{1b}E₂Eᵢᵢ | | E₅ | E_{f} |
| 2025 | E₁ₐE₂EᵢᵢE_{iib} | | E₅ | E_{f} |
| 2026 | E_{1b}E₂EᵢᵢE_{iib} | | E₅ | E_{f} |
| 2027 | E₁ₐE₂E₃Eᵢ | | E₅ | E_{f} |
| 2028 | E_{1b}E₂E₃Eᵢ | | E₅ | E_{f} |
| 2029 | E₁ₐE₂E₃Eᵢᵢ | | E₅ | E_{f} |
| 2030 | E_{1b}E₂E₃Eᵢᵢ | | E₅ | E_{f} |
| 2031 | E₁ₐE₂E₃EᵢᵢE_{iib} | | E₅ | E_{f} |
| 2032 | E_{1b}E₂E₃EᵢᵢE_{iib} | | E₅ | E_{f} |
| 2033 | E₁ₐE₂E₄ | | E₅ | E_{f} |
| 2034 | E_{1b}E₂E₄ | | E₅ | E_{f} |
| 2035 | E₁ₐE₂E₃E₄ | | E₅ | E_{f} |
| 2036 | E_{1b}E₂E₃E₄ | | E₅ | E_{f} |
| 2037 | E₁ₐE₂EᵢE₄ | | E₅ | E_{f} |
| 2038 | E_{1b}E₂EᵢE₄ | | E₅ | E_{f} |
| 2039 | E₁ₐE₂EᵢᵢE₄ | | E₅ | E_{f} |
| 2040 | E_{1b}E₂EᵢᵢE₄ | | E₅ | E_{f} |
| 2041 | E₁ₐE₂EᵢᵢE_{iib}E₄ | | E₅ | E_{f} |
| 2042 | E_{1b}E₂EᵢᵢE_{iib}E₄ | | E₅ | E_{f} |
| 2043 | E₁ₐE₂E₃EᵢE₄ | | E₅ | E_{f} |
| 2044 | E_{1b}E₂E₃EᵢE₄ | | E₅ | E_{f} |
| 2045 | E₁ₐE₂E₃EᵢᵢE₄ | | E₅ | E_{f} |
| 2046 | E_{1b}E₂E₃EᵢᵢE₄ | | E₅ | E_{f} |
| 2047 | E₁ₐE₂E₃EᵢᵢE_{iib}E₄ | | E₅ | E_{f} |
| 2048 | E_{1b}E₂E₃EᵢᵢE_{iib}E₄ | | E₅ | E_{f} |
| 2049 | E₁ₐE₂Eᵢᵢᵢ | | E₅ | E_{f} |
| 2050 | E_{1b}E₂Eᵢᵢᵢ | | E₅ | E_{f} |
| 2051 | E₁ₐE₂E₃Eᵢᵢᵢ | | E₅ | E_{f} |
| 2052 | E_{1b}E₂E₃Eᵢᵢᵢ | | E₅ | E_{f} |
| 2053 | E₁ₐE₂EᵢᵢᵢE₄ | | E₅ | E_{f} |
| 2054 | E_{1b}E₂EᵢᵢᵢE₄ | | E₅ | E_{f} |
| 2055 | E₁ₐE₂E₃EᵢᵢᵢE₄ | | E₅ | E_{f} |
| 2056 | E_{1b}E₂E₃EᵢᵢᵢE₄ | | E₅ | E_{f} |
| 2057 | E₁ₐE₂Eᵢᵥ | | E₅ | E_{f} |
| 2058 | E_{1b}E₂Eᵢᵥ | | E₅ | E_{f} |
| 2059 | E₁ₐE₂E₃Eᵢᵥ | | E₅ | E_{f} |
| 2060 | E_{1b}E₂E₃Eᵢᵥ | | E₅ | E_{f} |
| 2061 | E₁ₐE₂EᵢᵥE₄ | | E₅ | E_{f} |
| 2062 | E_{1b}E₂EᵢᵥE₄ | | E₅ | E_{f} |
| 2063 | E₁ₐE₂E₃EᵢᵥE₄ | | E₅ | E_{f} |
| 2064 | E_{1b}E₂E₃EᵢᵥE₄ | | E₅ | E_{f} |
| 2065 | E₁ₐE₂ | | E₄*E₅ | E_{f} |
| 2066 | E_{1b}E₂ | | E₄*E₅ | E_{f} |
| 2067 | E₁ₐE₂E₃ | | E₄*E₅ | E_{f} |
| 2068 | E_{1b}E₂E₃ | | E₄*E₅ | E_{f} |
| 2069 | E₁ₐE₂Eᵢ | | E₄*E₅ | E_{f} |
| 2070 | E_{1b}E₂Eᵢ | | E₄*E₅ | E_{f} |
| 2071 | E₁ₐE₂Eᵢᵢ | | E₄*E₅ | E_{f} |
| 2072 | E_{1b}E₂Eᵢᵢ | | E₄*E₅ | E_{f} |
| 2073 | E₁ₐE₂EᵢᵢE_{iib} | | E₄*E₅ | E_{f} |
| 2074 | E_{1b}E₂EᵢᵢE_{iib} | | E₄*E₅ | E_{f} |
| 2075 | E₁ₐE₂E₃Eᵢ | | E₄*E₅ | E_{f} |
| 2076 | E_{1b}E₂E₃Eᵢ | | E₄*E₅ | E_{f} |
| 2077 | E₁ₐE₂E₃Eᵢᵢ | | E₄*E₅ | E_{f} |
| 2078 | E_{1b}E₂E₃Eᵢᵢ | | E₄*E₅ | E_{f} |
| 2079 | E₁ₐE₂E₃EᵢᵢE_{iib} | | E₄*E₅ | E_{f} |
| 2080 | E_{1b}E₂E₃EᵢᵢE_{iib} | | E₄*E₅ | E_{f} |
| 2081 | E₁ₐE₂E₄ | | E₄*E₅ | E_{f} |
| 2082 | E_{1b}E₂E₄ | | E₄*E₅ | E_{f} |
| 2083 | E₁ₐE₂E₃E₄ | | E₄*E₅ | E_{f} |
| 2084 | E_{1b}E₂E₃E₄ | | E₄*E₅ | E_{f} |
| 2085 | E₁ₐE₂EᵢE₄ | | E₄*E₅ | E_{f} |
| 2086 | E_{1b}E₂EᵢE₄ | | E₄*E₅ | E_{f} |
| 2087 | E₁ₐE₂EᵢᵢE₄ | | E₄*E₅ | E_{f} |
| 2088 | E_{1b}E₂EᵢᵢE₄ | | E₄*E₅ | E_{f} |
| 2089 | E₁ₐE₂EᵢᵢE_{iib}E₄ | | E₄*E₅ | E_{f} |
| 2090 | E_{1b}E₂EᵢᵢE_{iib}E₄ | | E₄*E₅ | E_{f} |
| 2091 | E₁ₐE₂E₃EᵢE₄ | | E₄*E₅ | E_{f} |
| 2092 | E_{1b}E₂E₃EᵢE₄ | | E₄*E₅ | E_{f} |
| 2093 | E₁ₐE₂E₃EᵢᵢE₄ | | E₄*E₅ | E_{f} |
| 2094 | E_{1b}E₂E₃EᵢᵢE₄ | | E₄*E₅ | E_{f} |
| 2095 | E₁ₐE₂E₃EᵢᵢE_{iib}E₄ | | E₄*E₅ | E_{f} |
| 2096 | E_{1b}E₂E₃EᵢᵢE_{iib}E₄ | | E₄*E₅ | E_{f} |
| 2097 | E₁ₐE₂Eᵢᵢᵢ | | E₄*E₅ | E_{f} |
| 2098 | E_{1b}E₂Eᵢᵢᵢ | | E₄*E₅ | E_{f} |
| 2099 | E₁ₐE₂E₃Eᵢᵢᵢ | | E₄*E₅ | E_{f} |
| 2100 | E_{1b}E₂E₃Eᵢᵢᵢ | | E₄*E₅ | E_{f} |
| 2101 | E₁ₐE₂EᵢᵢᵢE₄ | | E₄*E₅ | E_{f} |
| 2102 | E_{1b}E₂EᵢᵢᵢE₄ | | E₄*E₅ | E_{f} |
| 2103 | E₁ₐE₂E₃EᵢᵢᵢE₄ | | E₄*E₅ | E_{f} |
| 2104 | E_{1b}E₂E₃EᵢᵢᵢE₄ | | E₄*E₅ | E_{f} |
| 2105 | E₁ₐE₂Eᵢᵥ | | E₄*E₅ | E_{f} |
| 2106 | E_{1b}E₂Eᵢᵥ | | E₄*E₅ | E_{f} |
| 2107 | E₁ₐE₂E₃Eᵢᵥ | | E₄*E₅ | E_{f} |
| 2108 | E_{1b}E₂E₃Eᵢᵥ | | E₄*E₅ | E_{f} |
| 2109 | E₁ₐE₂EᵢᵥE₄ | | E₄*E₅ | E_{f} |
| 2110 | E_{1b}E₂EᵢᵥE₄ | | E₄*E₅ | E_{f} |
| 2111 | E₁ₐE₂E₃EᵢᵥE₄ | | E₄*E₅ | E_{f} |
| 2112 | E_{1b}E₂E₃EᵢᵥE₄ | | E₄*E₅ | E_{f} |
| 2113 | E₁ₐE₂ | AlkL | | E_{f} |
| 2114 | E_{1b}E₂ | AlkL | | E_{f} |
| 2115 | E₁ₐE₂E₃ | AlkL | | E_{f} |
| 2116 | E_{1b}E₂E₃ | AlkL | | E_{f} |
| 2117 | E₁ₐE₂Eᵢ | AlkL | | E_{f} |
| 2118 | E_{1b}E₂Eᵢ | AlkL | | E_{f} |
| 2119 | E₁ₐE₂Eᵢᵢ | AlkL | | E_{f} |
| 2120 | E_{1b}E₂Eᵢᵢ | AlkL | | E_{f} |
| 2121 | E₁ₐE₂EᵢᵢE_{iib} | AlkL | | E_{f} |
| 2122 | E_{1b}E₂EᵢᵢE_{iib} | AlkL | | E_{f} |
| 2123 | E₁ₐE₂E₃Eᵢ | AlkL | | E_{f} |
| 2124 | E_{1b}E₂E₃Eᵢ | AlkL | | E_{f} |
| 2125 | E₁ₐE₂E₃Eᵢᵢ | AlkL | | E_{f} |
| 2126 | E_{1b}E₂E₃Eᵢᵢ | AlkL | | E_{f} |
| 2127 | E₁ₐE₂E₃EᵢᵢE_{iib} | AlkL | | E_{f} |
| 2128 | E_{1b}E₂E₃EᵢᵢE_{iib} | AlkL | | E_{f} |
| 2129 | E₁ₐE₂E₄ | AlkL | | E_{f} |
| 2130 | E_{1b}E₂E₄ | AlkL | | E_{f} |
| 2131 | E₁ₐE₂E₃E₄ | AlkL | | E_{f} |
| 2132 | E_{1b}E₂E₃E₄ | AlkL | | E_{f} |
| 2133 | E₁ₐE₂EᵢE₄ | AlkL | | E_{f} |
| 2134 | E_{1b}E₂EᵢE₄ | AlkL | | E_{f} |
| 2135 | E₁ₐE₂EᵢᵢE₄ | AlkL | | E_{f} |
| 2136 | E_{1b}E₂EᵢᵢE₄ | AlkL | | E_{f} |
| 2137 | E₁ₐE₂EᵢᵢE_{iib}E₄ | AlkL | | E_{f} |
| 2138 | E_{1b}E₂EᵢᵢE_{iib}E₄ | AlkL | | E_{f} |
| 2139 | E₁ₐE₂E₃EᵢE₄ | AlkL | | E_{f} |
| 2140 | E_{1b}E₂E₃EᵢE₄ | AlkL | | E_{f} |
| 2141 | E₁ₐE₂E₃EᵢᵢE₄ | AlkL | | E_{f} |
| 2142 | E_{1b}E₂E₃EᵢᵢE₄ | AlkL | | E_{f} |
| 2143 | E₁ₐE₂E₃EᵢᵢE_{iib}E₄ | AlkL | | E_{f} |
| 2144 | E_{1b}E₂E₃EᵢᵢE_{iib}E₄ | AlkL | | E_{f} |
| 2145 | E₁ₐE₂Eᵢᵢᵢ | AlkL | | E_{f} |
| 2146 | E_{1b}E₂Eᵢᵢᵢ | AlkL | | E_{f} |
| 2147 | E₁ₐE₂E₃Eᵢᵢᵢ | AlkL | | E_{f} |
| 2148 | E_{1b}E₂E₃Eᵢᵢᵢ | AlkL | | E_{f} |
| 2149 | E₁ₐE₂EᵢᵢᵢE₄ | AlkL | | E_{f} |
| 2150 | E_{1b}E₂EᵢᵢᵢE₄ | AlkL | | E_{f} |
| 2151 | E₁ₐE₂E₃EᵢᵢᵢE₄ | AlkL | | E_{f} |
| 2152 | E_{1b}E₂E₃EᵢᵢᵢE₄ | AlkL | | E_{f} |
| 2153 | E₁ₐE₂Eᵢᵥ | AlkL | | E_{f} |
| 2154 | E_{1b}E₂Eᵢᵥ | AlkL | | E_{f} |
| 2155 | E₁ₐE₂E₃Eᵢᵥ | AlkL | | E_{f} |
| 2156 | E_{1b}E₂E₃Eᵢᵥ | AlkL | | E_{f} |
| 2157 | E₁ₐE₂EᵢᵥE₄ | AlkL | | E_{f} |
| 2158 | E_{1b}E₂EᵢᵥE₄ | AlkL | | E_{f} |
| 2159 | E₁ₐE₂E₃EᵢᵥE₄ | AlkL | | E_{f} |
| 2160 | E_{1b}E₂E₃EᵢᵥE₄ | AlkL | | E_{f} |
| 2161 | E₁ₐE₂ | AlkL | E₄* | E_{f} |
| 2162 | E_{1b}E₂ | AlkL | E₄* | E_{f} |
| 2163 | E₁ₐE₂E₃ | AlkL | E₄* | E_{f} |
| 2164 | E_{1b}E₂E₃ | AlkL | E₄* | E_{f} |
| 2165 | E₁ₐE₂Eᵢ | AlkL | E₄* | E_{f} |
| 2166 | E_{1b}E₂Eᵢ | AlkL | E₄* | E_{f} |
| 2167 | E₁ₐE₂Eᵢᵢ | AlkL | E₄* | E_{f} |
| 2168 | E_{1b}E₂Eᵢᵢ | AlkL | E₄* | E_{f} |
| 2169 | E₁ₐE₂EᵢᵢE_{iib} | AlkL | E₄* | E_{f} |
| 2170 | E_{1b}E₂EᵢᵢE_{iib} | AlkL | E₄* | E_{f} |
| 2171 | E₁ₐE₂E₃Eᵢ | AlkL | E₄* | E_{f} |
| 2172 | E_{1b}E₂E₃Eᵢ | AlkL | E₄* | E_{f} |
| 2173 | E₁ₐE₂E₃Eᵢᵢ | AlkL | E₄* | E_{f} |
| 2174 | E_{1b}E₂E₃Eᵢᵢ | AlkL | E₄* | E_{f} |
| 2175 | E₁ₐE₂E₃EᵢᵢE_{iib} | AlkL | E₄* | E_{f} |
| 2176 | E_{1b}E₂E₃EᵢᵢE_{iib} | AlkL | E₄* | E_{f} |
| 2177 | E₁ₐE₂E₄ | AlkL | E₄* | E_{f} |
| 2178 | E_{1b}E₂E₄ | AlkL | E₄* | E_{f} |
| 2179 | E₁ₐE₂E₃E₄ | AlkL | E₄* | E_{f} |
| 2180 | E_{1b}E₂E₃E₄ | AlkL | E₄* | E_{f} |
| 2181 | E₁ₐE₂EᵢE₄ | AlkL | E₄* | E_{f} |
| 2182 | E_{1b}E₂EᵢE₄ | AlkL | E₄* | E_{f} |
| 2183 | E₁ₐE₂EᵢᵢE₄ | AlkL | E₄* | E_{f} |
| 2184 | E_{1b}E₂EᵢᵢE₄ | AlkL | E₄* | E_{f} |
| 2185 | E₁ₐE₂EᵢᵢE_{iib}E₄ | AlkL | E₄* | E_{f} |
| 2186 | E_{1b}E₂EᵢᵢE_{iib}E₄ | AlkL | E₄* | E_{f} |
| 2187 | E₁ₐE₂E₃EᵢE₄ | AlkL | E₄* | E_{f} |
| 2188 | E_{1b}E₂E₃EᵢE₄ | AlkL | E₄* | E_{f} |
| 2189 | E₁ₐE₂E₃EᵢᵢE₄ | AlkL | E₄* | E_{f} |
| 2190 | E_{1b}E₂E₃EᵢᵢE₄ | AlkL | E₄* | E_{f} |
| 2191 | E₁ₐE₂E₃EᵢᵢE_{iib}E₄ | AlkL | E₄* | E_{f} |
| 2192 | E_{1b}E₂E₃EᵢᵢE_{iib}E₄ | AlkL | E₄* | E_{f} |
| 2193 | E₁ₐE₂Eᵢᵢᵢ | AlkL | E₄* | E_{f} |
| 2194 | E_{1b}E₂Eᵢᵢᵢ | AlkL | E₄* | E_{f} |
| 2195 | E₁ₐE₂E₃Eᵢᵢᵢ | AlkL | E₄* | E_{f} |
| 2196 | E_{1b}E₂E₃Eᵢᵢᵢ | AlkL | E₄* | E_{f} |
| 2197 | E₁ₐE₂EᵢᵢᵢE₄ | AlkL | E₄* | E_{f} |
| 2198 | E_{1b}E₂EᵢᵢᵢE₄ | AlkL | E₄* | E_{f} |
| 2199 | E₁ₐE₂E₃EᵢᵢᵢE₄ | AlkL | E₄* | E_{f} |
| 2200 | E_{1b}E₂E₃EᵢᵢᵢE₄ | AlkL | E₄* | E_{f} |
| 2201 | E₁ₐE₂Eᵢᵥ | AlkL | E₄* | E_{f} |
| 2202 | E_{1b}E₂Eᵢᵥ | AlkL | E₄* | E_{f} |
| 2203 | E₁ₐE₂E₃Eᵢᵥ | AlkL | E₄* | E_{f} |
| 2204 | E_{1b}E₂E₃Eᵢᵥ | AlkL | E₄* | E_{f} |
| 2205 | E₁ₐE₂EᵢᵥE₄ | AlkL | E₄* | E_{f} |
| 2206 | E_{1b}E₂EᵢᵥE₄ | AlkL | E₄* | E_{f} |
| 2207 | E₁ₐE₂E₃EᵢᵥE₄ | AlkL | E₄* | E_{f} |
| 2208 | E_{1b}E₂E₃EᵢᵥE₄ | AlkL | E₄* | E_{f} |
| 2209 | E₁ₐE₂ | AlkL | E₅ | E_{f} |
| 2210 | E_{1b}E₂ | AlkL | E₅ | E_{f} |
| 2211 | E₁ₐE₂E₃ | AlkL | E₅ | E_{f} |
| 2212 | E_{1b}E₂E₃ | AlkL | E₅ | E_{f} |
| 2213 | E₁ₐE₂Eᵢ | AlkL | E₅ | E_{f} |
| 2214 | E_{1b}E₂Eᵢ | AlkL | E₅ | E_{f} |
| 2215 | E₁ₐE₂Eᵢᵢ | AlkL | E₅ | E_{f} |
| 2216 | E_{1b}E₂Eᵢᵢ | AlkL | E₅ | E_{f} |
| 2217 | E₁ₐE₂EᵢᵢE_{iib} | AlkL | E₅ | E_{f} |
| 2218 | E_{1b}E₂EᵢᵢE_{iib} | AlkL | E₅ | E_{f} |
| 2219 | E₁ₐE₂E₃Eᵢ | AlkL | E₅ | E_{f} |
| 2220 | E_{1b}E₂E₃Eᵢ | AlkL | E₅ | E_{f} |
| 2221 | E₁ₐE₂E₃Eᵢᵢ | AlkL | E₅ | E_{f} |
| 2222 | E_{1b}E₂E₃Eᵢᵢ | AlkL | E₅ | E_{f} |
| 2223 | E₁ₐE₂E₃EᵢᵢE_{iib} | AlkL | E₅ | E_{f} |
| 2224 | E_{1b}E₂E₃EᵢᵢE_{iib} | AlkL | E₅ | E_{f} |
| 2225 | E₁ₐE₂E₄ | AlkL | E₅ | E_{f} |
| 2226 | E_{1b}E₂E₄ | AlkL | E₅ | E_{f} |
| 2227 | E₁ₐE₂E₃E₄ | AlkL | E₅ | E_{f} |
| 2228 | E_{1b}E₂E₃E₄ | AlkL | E₅ | E_{f} |
| 2229 | E₁ₐE₂EᵢE₄ | AlkL | E₅ | E_{f} |
| 2230 | E_{1b}E₂EᵢE₄ | AlkL | E₅ | E_{f} |
| 2231 | E₁ₐE₂EᵢᵢE₄ | AlkL | E₅ | E_{f} |
| 2232 | E_{1b}E₂EᵢᵢE₄ | AlkL | E₅ | E_{f} |
| 2233 | E₁ₐE₂EᵢᵢE_{iib}E₄ | AlkL | E₅ | E_{f} |
| 2234 | E_{1b}E₂EᵢᵢE_{iib}E₄ | AlkL | E₅ | E_{f} |
| 2235 | E₁ₐE₂E₃EᵢE₄ | AlkL | E₅ | E_{f} |
| 2236 | E_{1b}E₂E₃EᵢE₄ | AlkL | E₅ | E_{f} |
| 2237 | E₁ₐE₂E₃EᵢᵢE₄ | AlkL | E₅ | E_{f} |
| 2238 | E_{1b}E₂E₃EᵢᵢE₄ | AlkL | E₅ | E_{f} |
| 2239 | E₁ₐE₂E₃EᵢᵢE_{iib}E₄ | AlkL | E₅ | E_{f} |
| 2240 | E_{1b}E₂E₃EᵢᵢE_{iib}E₄ | AlkL | E₅ | E_{f} |
| 2241 | E₁ₐE₂Eᵢᵢᵢ | AlkL | E₅ | E_{f} |
| 2242 | E_{1b}E₂Eᵢᵢᵢ | AlkL | E₅ | E_{f} |
| 2243 | E₁ₐE₂E₃Eᵢᵢᵢ | AlkL | E₅ | E_{f} |
| 2244 | E_{1b}E₂E₃Eᵢᵢᵢ | AlkL | E₅ | E_{f} |
| 2245 | E₁ₐE₂EᵢᵢᵢE₄ | AlkL | E₅ | E_{f} |
| 2246 | E_{1b}E₂EᵢᵢᵢE₄ | AlkL | E₅ | E_{f} |
| 2247 | E₁ₐE₂E₃EᵢᵢᵢE₄ | AlkL | E₅ | E_{f} |
| 2248 | E_{1b}E₂E₃EᵢᵢᵢE₄ | AlkL | E₅ | E_{f} |
| 2249 | E₁ₐE₂Eᵢᵥ | AlkL | E₅ | E_{f} |
| 2250 | E_{1b}E₂Eᵢᵥ | AlkL | E₅ | E_{f} |
| 2251 | E₁ₐE₂E₃Eᵢᵥ | AlkL | E₅ | E_{f} |
| 2252 | E_{1b}E₂E₃Eᵢᵥ | AlkL | E₅ | E_{f} |
| 2253 | E₁ₐE₂EᵢᵥE₄ | AlkL | E₅ | E_{f} |
| 2254 | E_{1b}E₂EᵢᵥE₄ | AlkL | E₅ | E_{f} |
| 2255 | E₁ₐE₂E₃EᵢᵥE₄ | AlkL | E₅ | E_{f} |
| 2256 | E_{1b}E₂E₃EᵢᵥE₄ | AlkL | E₅ | E_{f} |
| 2257 | E₁ₐE₂ | AlkL | E₄*E₅ | E_{f} |
| 2258 | E_{1b}E₂ | AlkL | E₄*E₅ | E_{f} |
| 2259 | E₁ₐE₂E₃ | AlkL | E₄*E₅ | E_{f} |
| 2260 | E_{1b}E₂E₃ | AlkL | E₄*E₅ | E_{f} |
| 2261 | E₁ₐE₂Eᵢ | AlkL | E₄*E₅ | E_{f} |
| 2262 | E_{1b}E₂Eᵢ | AlkL | E₄*E₅ | E_{f} |
| 2263 | E₁ₐE₂Eᵢᵢ | AlkL | E₄*E₅ | E_{f} |
| 2264 | E_{1b}E₂Eᵢᵢ | AlkL | E₄*E₅ | E_{f} |
| 2265 | E₁ₐE₂EᵢᵢE_{iib} | AlkL | E₄*E₅ | E_{f} |
| 2266 | E_{1b}E₂EᵢᵢE_{iib} | AlkL | E₄*E₅ | E_{f} |
| 2267 | E₁ₐE₂E₃Eᵢ | AlkL | E₄*E₅ | E_{f} |
| 2268 | E_{1b}E₂E₃Eᵢ | AlkL | E₄*E₅ | E_{f} |
| 2269 | E₁ₐE₂E₃Eᵢᵢ | AlkL | E₄*E₅ | E_{f} |
| 2270 | E_{1b}E₂E₃Eᵢᵢ | AlkL | E₄*E₅ | E_{f} |
| 2271 | E₁ₐE₂E₃EᵢᵢE_{iib} | AlkL | E₄*E₅ | E_{f} |
| 2272 | E_{1b}E₂E₃EᵢᵢE_{iib} | AlkL | E₄*E₅ | E_{f} |
| 2273 | E₁ₐE₂E₄ | AlkL | E₄*E₅ | E_{f} |
| 2274 | E_{1b}E₂E₄ | AlkL | E₄*E₅ | E_{f} |
| 2275 | E₁ₐE₂E₃E₄ | AlkL | E₄*E₅ | E_{f} |
| 2276 | E_{1b}E₂E₃E₄ | AlkL | E₄*E₅ | E_{f} |
| 2277 | E₁ₐE₂EᵢE₄ | AlkL | E₄*E₅ | E_{f} |
| 2278 | E_{1b}E₂EᵢE₄ | AlkL | E₄*E₅ | E_{f} |
| 2279 | E₁ₐE₂EᵢᵢE₄ | AlkL | E₄*E₅ | E_{f} |
| 2280 | E_{1b}E₂EᵢᵢE₄ | AlkL | E₄*E₅ | E_{f} |
| 2281 | E₁ₐE₂EᵢᵢE_{iib}E₄ | AlkL | E₄*E₅ | E_{f} |
| 2282 | E_{1b}E₂EᵢᵢE_{iib}E₄ | AlkL | E₄*E₅ | E_{f} |
| 2283 | E₁ₐE₂E₃EᵢE₄ | AlkL | E₄*E₅ | E_{f} |
| 2284 | E_{1b}E₂E₃EᵢE₄ | AlkL | E₄*E₅ | E_{f} |
| 2285 | E₁ₐE₂E₃EᵢᵢE₄ | AlkL | E₄*E₅ | E_{f} |
| 2286 | E_{1b}E₂E₃EᵢᵢE₄ | AlkL | E₄*E₅ | E_{f} |
| 2287 | E₁ₐE₂E₃EᵢᵢE_{iib}E₄ | AlkL | E₄*E₅ | E_{f} |
| 2288 | E_{1b}E₂E₃EᵢᵢE_{iib}E₄ | AlkL | E₄*E₅ | E_{f} |
| 2289 | E₁ₐE₂Eᵢᵢᵢ | AlkL | E₄*E₅ | E_{f} |
| 2290 | E_{1b}E₂Eᵢᵢᵢ | AlkL | E₄*E₅ | E_{f} |
| 2291 | E₁ₐE₂E₃Eᵢᵢᵢ | AlkL | E₄*E₅ | E_{f} |
| 2292 | E_{1b}E₂E₃Eᵢᵢᵢ | AlkL | E₄*E₅ | E_{f} |
| 2293 | E₁ₐE₂EᵢᵢᵢE₄ | AlkL | E₄*E₅ | E_{f} |
| 2294 | E_{1b}E₂EᵢᵢᵢE₄ | AlkL | E₄*E₅ | E_{f} |
| 2295 | E₁ₐE₂E₃EᵢᵢᵢE₄ | AlkL | E₄*E₅ | E_{f} |
| 2296 | E_{1b}E₂E₃EᵢᵢᵢE₄ | AlkL | E₄*E₅ | E_{f} |
| 2297 | E₁ₐE₂Eᵢᵥ | AlkL | E₄*E₅ | E_{f} |
| 2298 | E_{1b}E₂Eᵢᵥ | AlkL | E₄*E₅ | E_{f} |
| 2299 | E₁ₐE₂E₃Eᵢᵥ | AlkL | E₄*E₅ | E_{f} |
| 2300 | E_{1b}E₂E₃Eᵢᵥ | AlkL | E₄*E₅ | E_{f} |
| 2301 | E₁ₐE₂EᵢᵥE₄ | AlkL | E₄*E₅ | E_{f} |
| 2302 | E_{1b}E₂EᵢᵥE₄ | AlkL | E₄*E₅ | E_{f} |
| 2303 | E₁ₐE₂E₃EᵢᵥE₄ | AlkL | E₄*E₅ | E_{f} |
| 2304 | E_{1b}E₂E₃EᵢᵥE₄ | AlkL | E₄*E₅ | E_{f} |

Microorganisms quite especially preferred according to the invention (abbreviated as MO) are outstandingly suitable for the production of n-aminocarboxylate esters and have increased or decreased enzyme activities (abbreviated as E) described in the following table, where these can in addition advantageously be combined with an increased enzyme activity compared to the wild type of the microorganism, which is described for the 3-ketoacyl-ACP (Acyl Carrrier Protein) synthase III (EC 2.3.1.41), in particular that from plants, preferably that from plants the seeds whereof contain fatty acids with alkyl residues shorter than 14 C atoms, and particularly preferably that from plants of the genera Cuphea, Elaeis, Cocos, Umbellularia and Cinnamomum and gene products selected from AccA, AccB, AccC, AccD, AceE, AceF, Lpd, AcpP, FabA, FabB, FabD, FabF, FabG, FabH, Fabl, FabZ, PanD, PanK, UdhA, PntA or PntB. Any combinations of at least two of these enzyme activities can advantageously be increased.

In addition, moreover, it can be advantageous if the microorganism is provided with a lower enzyme activity compared to the wild type of the microorganism, which is described for the gene products selected from TdcE, PflA, PflB, PflC, PflD, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, singly or in any combination.

| | | |
|---|---|---|
| MO | E increased | E decreased |

| | | | | |
|---|---|---|---|---|
| 1 | E₁ₐE₂ | EᵥEᵥᵢ | | |
| 2 | E_{1b}E₂ | EᵥEᵥᵢ | | |
| 3 | E₁ₐE₂E₃ | EᵥEᵥᵢ | | |
| 4 | E_{1b}E₂E₃ | EᵥEᵥᵢ | | |
| 5 | E₁ₐE₂Eᵢ | EᵥEᵥᵢ | | |
| 6 | E_{1b}E₂Eᵢ | EᵥEᵥᵢ | | |
| 7 | E₁ₐE₂Eᵢᵢ | EᵥEᵥᵢ | | |
| 8 | E_{1b}E₂Eᵢᵢ | EᵥEᵥᵢ | | |
| 9 | E₁ₐE₂EᵢᵢE_{iib} | EᵥEᵥᵢ | | |
| 10 | E_{1b}E₂EᵢᵢE_{iib} | EᵥEᵥᵢ | | |
| 11 | E₁ₐE₂E₃Eᵢ | EᵥEᵥᵢ | | |
| 12 | E_{1b}E₂E₃Eᵢ | EᵥEᵥᵢ | | |
| 13 | E₁ₐE₂E₃Eᵢᵢ | EᵥEᵥᵢ | | |
| 14 | E_{1b}E₂E₃Eᵢᵢ | EᵥEᵥᵢ | | |
| 15 | E₁ₐE₂E₃EᵢᵢE_{iib} | EᵥEᵥᵢ | | |
| 16 | E_{1b}E₂E₃EᵢᵢE_{iib} | EᵥEᵥᵢ | | |
| 17 | E₁ₐE₂E₄ | EᵥEᵥᵢ | | |
| 18 | E_{1b}E₂E₄ | EᵥEᵥᵢ | | |
| 19 | E₁ₐE₂E₃E₄ | EᵥEᵥᵢ | | |
| 20 | E_{1b}E₂E₃E₄ | EᵥEᵥᵢ | | |
| 21 | E₁ₐE₂EᵢE₄ | EᵥEᵥᵢ | | |
| 22 | E_{1b}E₂EᵢE₄ | EᵥEᵥᵢ | | |
| 23 | E₁ₐE₂EᵢᵢE₄ | EᵥEᵥᵢ | | |
| 24 | E_{1b}E₂EᵢᵢE₄ | EᵥEᵥᵢ | | |
| 25 | E₁ₐE₂EᵢᵢE_{iib}E₄ | EᵥEᵥᵢ | | |
| 26 | E_{1b}E₂EᵢᵢE_{iib}E₄ | EᵥEᵥᵢ | | |
| 27 | E₁ₐE₂E₃EᵢE₄ | EᵥEᵥᵢ | | |
| 28 | E_{1b}E₂E₃EᵢE₄ | EᵥEᵥᵢ | | |
| 29 | E₁ₐE₂E₃EᵢᵢE₄ | EᵥEᵥᵢ | | |
| 30 | E_{1b}E₂E₃EᵢᵢE₄ | EᵥEᵥᵢ | | |
| 31 | E₁ₐE₂E₃EᵢᵢE_{iib}E₄ | EᵥEᵥᵢ | | |
| 32 | E_{1b}E₂E₃EᵢᵢE_{iib}E₄ | EᵥEᵥᵢ | | |
| 33 | E₁ₐE₂Eᵢᵢᵢ | EᵥEᵥᵢ | | |
| 34 | E_{1b}E₂Eᵢᵢᵢ | EᵥEᵥᵢ | | |
| 35 | E₁ₐE₂E₃Eᵢᵢᵢ | EᵥEᵥᵢ | | |
| 36 | E_{1b}E₂E₃Eᵢᵢᵢ | EᵥEᵥᵢ | | |
| 37 | E₁ₐE₂EᵢᵢᵢE₄ | EᵥEᵥᵢ | | |
| 38 | E_{1b}E₂EᵢᵢᵢE₄ | EᵥEᵥᵢ | | |
| 39 | E₁ₐE₂E₃EᵢᵢᵢE₄ | EᵥEᵥᵢ | | |
| 40 | E_{1b}E₂E₃EᵢᵢᵢE₄ | EᵥEᵥᵢ | | |
| 41 | E₁ₐE₂Eᵢᵥ | EᵥEᵥᵢ | | |
| 42 | E_{1b}E₂Eᵢᵥ | EᵥEᵥᵢ | | |
| 43 | E₁ₐE₂E₃Eᵢᵥ | EᵥEᵥᵢ | | |
| 44 | E_{1b}E₂E₃Eᵢᵥ | EᵥEᵥᵢ | | |
| 45 | E₁ₐE₂EᵢᵥE₄ | EᵥEᵥᵢ | | |
| 46 | E_{1b}E₂EᵢᵥE₄ | EᵥEᵥᵢ | | |
| 47 | E₁ₐE₂E₃EᵢᵥE₄ | EᵥEᵥᵢ | | |
| 48 | E_{1b}E₂E₃EᵢᵥE₄ | EᵥEᵥᵢ | | |
| 49 | E₁ₐE₂ | EᵥEᵥᵢ | E₄* | |
| 50 | E_{1b}E₂ | EᵥEᵥᵢ | E₄* | |
| 51 | E₁ₐE₂E₃ | EᵥEᵥᵢ | E₄* | |
| 52 | E_{1b}E₂E₃ | EᵥEᵥᵢ | E₄* | |
| 53 | E₁ₐE₂Eᵢ | EᵥEᵥᵢ | E₄* | |
| 54 | E_{1b}E₂Eᵢ | EᵥEᵥᵢ | E₄* | |
| 55 | E₁ₐE₂Eᵢᵢ | EᵥEᵥᵢ | E₄* | |
| 56 | E_{1b}E₂Eᵢᵢ | EᵥEᵥᵢ | E₄* | |
| 57 | E₁ₐE₂EᵢᵢE_{iib} | EᵥEᵥᵢ | E₄* | |
| 58 | E_{1b}E₂EᵢᵢE_{iib} | EᵥEᵥᵢ | E₄* | |
| 59 | E₁ₐE₂E₃Eᵢ | EᵥEᵥᵢ | E₄* | |
| 60 | E_{1b}E₂E₃Eᵢ | EᵥEᵥᵢ | E₄* | |
| 61 | E₁ₐE₂E₃Eᵢᵢ | EᵥEᵥᵢ | E₄* | |
| 62 | E_{1b}E₂E₃Eᵢᵢ | EᵥEᵥᵢ | E₄* | |
| 63 | E₁ₐE₂E₃EᵢᵢE_{iib} | EᵥEᵥᵢ | E₄* | |
| 64 | E_{1b}E₂E₃EᵢᵢE_{iib} | EᵥEᵥᵢ | E₄* | |
| 65 | E₁ₐE₂E₄ | EᵥEᵥᵢ | E₄* | |
| 66 | E_{1b}E₂E₄ | EᵥEᵥᵢ | E₄* | |
| 67 | E₁ₐE₂E₃E₄ | EᵥEᵥᵢ | E₄* | |
| 68 | E_{1b}E₂E₃E₄ | EᵥEᵥᵢ | E₄* | |
| 69 | E₁ₐE₂EᵢE₄ | EᵥEᵥᵢ | E₄* | |
| 70 | E_{1b}E₂EᵢE₄ | EᵥEᵥᵢ | E₄* | |
| 71 | E₁ₐE₂EᵢᵢE₄ | EᵥEᵥᵢ | E₄* | |
| 72 | E_{1b}E₂EᵢᵢE₄ | EᵥEᵥᵢ | E₄* | |
| 73 | E₁ₐE₂EᵢᵢE_{iib}E₄ | EᵥEᵥᵢ | E₄* | |
| 74 | E_{1b}E₂EᵢᵢE_{iib}E₄ | EᵥEᵥᵢ | E₄* | |
| 75 | E₁ₐE₂E₃EᵢE₄ | EᵥEᵥᵢ | E₄* | |
| 76 | E_{1b}E₂E₃EᵢE₄ | EᵥEᵥᵢ | E₄* | |
| 77 | E₁ₐE₂E₃EᵢᵢE₄ | EᵥEᵥᵢ | E₄* | |
| 78 | E_{1b}E₂E₃EᵢᵢE₄ | EᵥEᵥᵢ | E₄* | |
| 79 | E₁ₐE₂E₃EᵢᵢE_{iib}E₄ | EᵥEᵥᵢ | E₄* | |
| 80 | E_{1b}E₂E₃EᵢᵢE_{iib}E₄ | EᵥEᵥᵢ | E₄* | |
| 81 | E₁ₐE₂Eᵢᵢᵢ | EᵥEᵥᵢ | E₄* | |
| 82 | E_{1b}E₂Eᵢᵢᵢ | EᵥEᵥᵢ | E₄* | |
| 83 | E₁ₐE₂E₃Eᵢᵢᵢ | EᵥEᵥᵢ | E₄* | |
| 84 | E_{1b}E₂E₃Eᵢᵢᵢ | EᵥEᵥᵢ | E₄* | |
| 85 | E₁ₐE₂EᵢᵢᵢE₄ | EᵥEᵥᵢ | E₄* | |
| 86 | E_{1b}E₂EᵢᵢᵢE₄ | EᵥEᵥᵢ | E₄* | |
| 87 | E₁ₐE₂E₃EᵢᵢᵢE₄ | EᵥEᵥᵢ | E₄* | |
| 88 | E_{1b}E₂E₃EᵢᵢᵢE₄ | EᵥEᵥᵢ | E₄* | |
| 89 | E₁ₐE₂Eᵢᵥ | EᵥEᵥᵢ | E₄* | |
| 90 | E_{1b}E₂Eᵢᵥ | EᵥEᵥᵢ | E₄* | |
| 91 | E₁ₐE₂E₃Eᵢᵥ | EᵥEᵥᵢ | E₄* | |
| 92 | E_{1b}E₂E₃Eᵢᵥ | EᵥEᵥᵢ | E₄* | |
| 93 | E₁ₐE₂EᵢᵥE₄ | EᵥEᵥᵢ | E₄* | |
| 94 | E_{1b}E₂EᵢᵥE₄ | EᵥEᵥᵢ | E₄* | |
| 95 | E₁ₐE₂E₃EᵢᵥE₄ | EᵥEᵥᵢ | E₄* | |
| 96 | E_{1b}E₂E₃EᵢᵥE₄ | EᵥEᵥᵢ | E₄* | |
| 97 | E₁ₐE₂ | EᵥEᵥᵢ | E₅ | |
| 98 | E_{1b}E₂ | EᵥEᵥᵢ | E₅ | |
| 99 | E₁ₐE₂E₃ | EᵥEᵥᵢ | E₅ | |
| 100 | E_{1b}E₂E₃ | EᵥEᵥᵢ | E₅ | |
| 101 | E₁ₐE₂Eᵢ | EᵥEᵥᵢ | E₅ | |
| 102 | E_{1b}E₂Eᵢ | EᵥEᵥᵢ | E₅ | |
| 103 | E₁ₐE₂Eᵢᵢ | EᵥEᵥᵢ | E₅ | |
| 104 | E_{1b}E₂Eᵢᵢ | EᵥEᵥᵢ | E₅ | |
| 105 | E₁ₐE₂EᵢᵢE_{iib} | EᵥEᵥᵢ | E₅ | |
| 106 | E_{1b}E₂EᵢᵢE_{iib} | EᵥEᵥᵢ | E₅ | |
| 107 | E₁ₐE₂E₃Eᵢ | EᵥEᵥᵢ | E₅ | |
| 108 | E_{1b}E₂E₃Eᵢ | EᵥEᵥᵢ | E₅ | |
| 109 | E₁ₐE₂E₃Eᵢᵢ | EᵥEᵥᵢ | E₅ | |
| 110 | E_{1b}E₂E₃Eᵢᵢ | EᵥEᵥᵢ | E₅ | |
| 111 | E₁ₐE₂E₃EᵢᵢE_{iib} | EᵥEᵥᵢ | E₅ | |
| 112 | E_{1b}E₂E₃EᵢᵢE_{iib} | EᵥEᵥᵢ | E₅ | |
| 113 | E₁ₐE₂E₄ | EᵥEᵥᵢ | E₅ | |
| 114 | E_{1b}E₂E₄ | EᵥEᵥᵢ | E₅ | |
| 115 | E₁ₐE₂E₃E₄ | EᵥEᵥᵢ | E₅ | |
| 116 | E_{1b}E₂E₃E₄ | EᵥEᵥᵢ | E₅ | |
| 117 | E₁ₐE₂EᵢE₄ | EᵥEᵥᵢ | E₅ | |
| 118 | E_{1b}E₂EᵢE₄ | EᵥEᵥᵢ | E₅ | |
| 119 | E₁ₐE₂EᵢᵢE₄ | EᵥEᵥᵢ | E₅ | |
| 120 | E_{1b}E₂EᵢᵢE₄ | EᵥEᵥᵢ | E₅ | |
| 121 | E₁ₐE₂EᵢᵢE_{iib}E₄ | EᵥEᵥᵢ | E₅ | |
| 122 | E_{1b}E₂EᵢᵢE_{iib}E₄ | EᵥEᵥᵢ | E₅ | |
| 123 | E₁ₐE₂E₃EᵢE₄ | EᵥEᵥᵢ | E₅ | |
| 124 | E_{1b}E₂E₃EᵢE₄ | EᵥEᵥᵢ | E₅ | |
| 125 | E₁ₐE₂E₃EᵢᵢE₄ | EᵥEᵥᵢ | E₅ | |
| 126 | E_{1b}E₂E₃EᵢᵢE₄ | EᵥEᵥᵢ | E₅ | |
| 127 | E₁ₐE₂E₃EᵢᵢE_{iib}E₄ | EᵥEᵥᵢ | E₅ | |
| 128 | E_{1b}E₂E₃EᵢᵢE_{iib}E₄ | EᵥEᵥᵢ | E₅ | |
| 129 | E₁ₐE₂Eᵢᵢᵢ | EᵥEᵥᵢ | E₅ | |
| 130 | E_{1b}E₂Eᵢᵢᵢ | EᵥEᵥᵢ | E₅ | |
| 131 | E₁ₐE₂E₃Eᵢᵢᵢ | EᵥEᵥᵢ | E₅ | |
| 132 | E_{1b}E₂E₃Eᵢᵢᵢ | EᵥEᵥᵢ | E₅ | |
| 133 | E₁ₐE₂EᵢᵢᵢE₄ | EᵥEᵥᵢ | E₅ | |
| 134 | E_{1b}E₂EᵢᵢᵢE₄ | EᵥEᵥᵢ | E₅ | |
| 135 | E₁ₐE₂E₃EᵢᵢᵢE₄ | EᵥEᵥᵢ | E₅ | |
| 136 | E_{1b}E₂E₃EᵢᵢᵢE₄ | EᵥEᵥᵢ | E₅ | |
| 137 | E₁ₐE₂Eᵢᵥ | EᵥEᵥᵢ | E₅ | |
| 138 | E_{1b}E₂Eᵢᵥ | EᵥEᵥᵢ | E₅ | |
| 139 | E₁ₐE₂E₃Eᵢᵥ | EᵥEᵥᵢ | E₅ | |
| 140 | E_{1b}E₂E₃Eᵢᵥ | EᵥEᵥᵢ | E₅ | |
| 141 | E₁ₐE₂EᵢᵥE₄ | EᵥEᵥᵢ | E₅ | |
| 142 | E_{1b}E₂EᵢᵥE₄ | EᵥEᵥᵢ | E₅ | |
| 143 | E₁ₐE₂E₃EᵢᵥE₄ | EᵥEᵥᵢ | E₅ | |
| 144 | E_{1b}E₂E₃EᵢᵥE₄ | EᵥEᵥᵢ | E₅ | |
| 145 | E₁ₐE₂ | EᵥEᵥᵢ | E₄*E₅ | |
| 146 | E_{1b}E₂ | EᵥEᵥᵢ | E₄*E₅ | |
| 147 | E₁ₐE₂E₃ | EᵥEᵥᵢ | E₄*E₅ | |
| 148 | E_{1b}E₂E₃ | EᵥEᵥᵢ | E₄*E₅ | |
| 149 | E₁ₐE₂Eᵢ | EᵥEᵥᵢ | E₄*E₅ | |
| 150 | E_{1b}E₂Eᵢ | EᵥEᵥᵢ | E₄*E₅ | |
| 151 | E₁ₐE₂Eᵢᵢ | EᵥEᵥᵢ | E₄*E₅ | |
| 152 | E_{1b}E₂Eᵢᵢ | EᵥEᵥᵢ | E₄*E₅ | |
| 153 | E₁ₐE₂EᵢᵢE_{iib} | EᵥEᵥᵢ | E₄*E₅ | |
| 154 | E_{1b}E₂EᵢᵢE_{iib} | EᵥEᵥᵢ | E₄*E₅ | |
| 155 | E₁ₐE₂E₃Eᵢ | EᵥEᵥᵢ | E₄*E₅ | |
| 156 | E_{1b}E₂E₃Eᵢ | EᵥEᵥᵢ | E₄*E₅ | |
| 157 | E₁ₐE₂E₃Eᵢᵢ | EᵥEᵥᵢ | E₄*E₅ | |
| 158 | E_{1b}E₂E₃Eᵢᵢ | EᵥEᵥᵢ | E₄*E₅ | |
| 159 | E₁ₐE₂E₃EᵢᵢE_{iib} | EᵥEᵥᵢ | E₄*E₅ | |
| 160 | E_{1b}E₂E₃EᵢᵢE_{iib} | EᵥEᵥᵢ | E₄*E₅ | |
| 161 | E₁ₐE₂E₄ | EᵥEᵥᵢ | E₄*E₅ | |
| 162 | E_{1b}E₂E₄ | EᵥEᵥᵢ | E₄*E₅ | |
| 163 | E₁ₐE₂E₃E₄ | EᵥEᵥᵢ | E₄*E₅ | |
| 164 | E_{1b}E₂E₃E₄ | EᵥEᵥᵢ | E₄*E₅ | |
| 165 | E₁ₐE₂EᵢE₄ | EᵥEᵥᵢ | E₄*E₅ | |
| 166 | E_{1b}E₂EᵢE₄ | EᵥEᵥᵢ | E₄*E₅ | |
| 167 | E₁ₐE₂EᵢᵢE₄ | EᵥEᵥᵢ | E₄*E₅ | |
| 168 | E_{1b}E₂EᵢᵢE₄ | EᵥEᵥᵢ | E₄*E₅ | |
| 169 | E₁ₐE₂EᵢᵢE_{iib}E₄ | EᵥEᵥᵢ | E₄*E₅ | |
| 170 | E_{1b}E₂EᵢᵢE_{iib}E₄ | EᵥEᵥᵢ | E₄*E₅ | |
| 171 | E₁ₐE₂E₃EᵢE₄ | EᵥEᵥᵢ | E₄*E₅ | |
| 172 | E_{1b}E₂E₃EᵢE₄ | EᵥEᵥᵢ | E₄*E₅ | |
| 173 | E₁ₐE₂E₃EᵢᵢE₄ | EᵥEᵥᵢ | E₄*E₅ | |
| 174 | E_{1b}E₂E₃EᵢᵢE₄ | EᵥEᵥᵢ | E₄*E₅ | |
| 175 | E₁ₐE₂E₃EᵢᵢE_{iib}E₄ | EᵥEᵥᵢ | E₄*E₅ | |
| 176 | E_{1b}E₂E₃EᵢᵢE_{iib}E₄ | EᵥEᵥᵢ | E₄*E₅ | |
| 177 | E₁ₐE₂Eᵢᵢᵢ | EᵥEᵥᵢ | E₄*E₅ | |
| 178 | E_{1b}E₂Eᵢᵢᵢ | EᵥEᵥᵢ | E₄*E₅ | |
| 179 | E₁ₐE₂E₃Eᵢᵢᵢ | EᵥEᵥᵢ | E₄*E₅ | |
| 180 | E_{1b}E₂E₃Eᵢᵢᵢ | EᵥEᵥᵢ | E₄*E₅ | |
| 181 | E₁ₐE₂EᵢᵢᵢE₄ | EᵥEᵥᵢ | E₄*E₅ | |
| 182 | E_{1b}E₂EᵢᵢᵢE₄ | EᵥEᵥᵢ | E₄*E₅ | |
| 183 | E₁ₐE₂E₃EᵢᵢᵢE₄ | EᵥEᵥᵢ | E₄*E₅ | |
| 184 | E_{1b}E₂E₃EᵢᵢᵢE₄ | EᵥEᵥᵢ | E₄*E₅ | |
| 185 | E₁ₐE₂Eᵢᵥ | EᵥEᵥᵢ | E₄*E₅ | |
| 186 | E_{1b}E₂Eᵢᵥ | EᵥEᵥᵢ | E₄*E₅ | |
| 187 | E₁ₐE₂E₃Eᵢᵥ | EᵥEᵥᵢ | E₄*E₅ | |
| 188 | E_{1b}E₂E₃Eᵢᵥ | EᵥEᵥᵢ | E₄*E₅ | |
| 189 | E₁ₐE₂EᵢᵥE₄ | EᵥEᵥᵢ | E₄*E₅ | |
| 190 | E_{1b}E₂EᵢᵥE₄ | EᵥEᵥᵢ | E₄*E₅ | |
| 191 | E₁ₐE₂E₃EᵢᵥE₄ | EᵥEᵥᵢ | E₄*E₅ | |
| 192 | E_{1b}E₂E₃EᵢᵥE₄ | EᵥEᵥᵢ | E₄*E₅ | |
| 193 | E₁ₐE₂ | AlkLEᵥEᵥᵢ | | |
| 194 | E_{1b}E₂ | AlkLEᵥEᵥᵢ | | |
| 195 | E₁ₐE₂E₃ | AlkLEᵥEᵥᵢ | | |
| 196 | E_{1b}E₂E₃ | AlkLEᵥEᵥᵢ | | |
| 197 | E₁ₐE₂Eᵢ | AlkLEᵥEᵥᵢ | | |
| 198 | E_{1b}E₂Eᵢ | AlkLEᵥEᵥᵢ | | |
| 199 | E₁ₐE₂Eᵢᵢ | AlkLEᵥEᵥᵢ | | |
| 200 | E_{1b}E₂Eᵢᵢ | AlkLEᵥEᵥᵢ | | |
| 201 | E₁ₐE₂EᵢᵢE_{iib} | AlkLEᵥEᵥᵢ | | |
| 202 | E_{1b}E₂EᵢᵢE_{iib} | AlkLEᵥEᵥᵢ | | |
| 203 | E₁ₐE₂E₃Eᵢ | AlkLEᵥEᵥᵢ | | |
| 204 | E_{1b}E₂E₃Eᵢ | AlkLEᵥEᵥᵢ | | |
| 205 | E₁ₐE₂E₃Eᵢᵢ | AlkLEᵥEᵥᵢ | | |
| 206 | E_{1b}E₂E₃Eᵢᵢ | AlkLEᵥEᵥᵢ | | |
| 207 | E₁ₐE₂E₃EᵢᵢE_{iib} | AlkLEᵥEᵥᵢ | | |
| 208 | E_{1b}E₂E₃EᵢᵢE_{iib} | AlkLEᵥEᵥᵢ | | |
| 209 | E₁ₐE₂E₄ | AlkLEᵥEᵥᵢ | | |
| 210 | E_{1b}E₂E₄ | AlkLEᵥEᵥᵢ | | |
| 211 | E₁ₐE₂E₃E₄ | AlkLEᵥEᵥᵢ | | |
| 212 | E_{1b}E₂E₃E₄ | AlkLEᵥEᵥᵢ | | |
| 213 | E₁ₐE₂EᵢE₄ | AlkLEᵥEᵥᵢ | | |
| 214 | E_{1b}E₂EᵢE₄ | AlkLEᵥEᵥᵢ | | |
| 215 | E₁ₐE₂EᵢᵢE₄ | AlkLEᵥEᵥᵢ | | |
| 216 | E_{1b}E₂EᵢᵢE₄ | AlkLEᵥEᵥᵢ | | |
| 217 | E₁ₐE₂EᵢᵢE_{iib}E₄ | AlkLEᵥEᵥᵢ | | |
| 218 | E_{1b}E₂EᵢᵢE_{iib}E₄ | AlkLEᵥEᵥᵢ | | |
| 219 | E₁ₐE₂E₃EᵢE₄ | AlkLEᵥEᵥᵢ | | |
| 220 | E_{1b}E₂E₃EᵢE₄ | AlkLEᵥEᵥᵢ | | |
| 221 | E₁ₐE₂E₃EᵢᵢE₄ | AlkLEᵥEᵥᵢ | | |
| 222 | E_{1b}E₂E₃EᵢᵢE₄ | AlkLEᵥEᵥᵢ | | |
| 223 | E₁ₐE₂E₃EᵢᵢE_{iib}E₄ | AlkLEᵥEᵥᵢ | | |
| 224 | E_{1b}E₂E₃EᵢᵢE_{iib}E₄ | AlkLEᵥEᵥᵢ | | |
| 225 | E₁ₐE₂Eᵢᵢᵢ | AlkLEᵥEᵥᵢ | | |
| 226 | E_{1b}E₂Eᵢᵢᵢ | AlkLEᵥEᵥᵢ | | |
| 227 | E₁ₐE₂E₃Eᵢᵢᵢ | AlkLEᵥEᵥᵢ | | |
| 228 | E_{1b}E₂E₃Eᵢᵢᵢ | AlkLEᵥEᵥᵢ | | |
| 229 | E₁ₐE₂EᵢᵢᵢE₄ | AlkLEᵥEᵥᵢ | | |
| 230 | E_{1b}E₂EᵢᵢᵢE₄ | AlkLEᵥEᵥᵢ | | |
| 231 | E₁ₐE₂E₃EᵢᵢᵢE₄ | AlkLEᵥEᵥᵢ | | |
| 232 | E_{1b}E₂E₃EᵢᵢᵢE₄ | AlkLEᵥEᵥᵢ | | |
| 233 | E₁ₐE₂Eᵢᵥ | AlkLEᵥEᵥᵢ | | |
| 234 | E_{1b}E₂Eᵢᵥ | AlkLEᵥEᵥᵢ | | |
| 235 | E₁ₐE₂E₃Eᵢᵥ | AlkLEᵥEᵥᵢ | | |
| 236 | E_{1b}E₂E₃Eᵢᵥ | AlkLEᵥEᵥᵢ | | |
| 237 | E₁ₐE₂EᵢᵥE₄ | AlkLEᵥEᵥᵢ | | |
| 238 | E_{1b}E₂EᵢᵥE₄ | AlkLEᵥEᵥᵢ | | |
| 239 | E₁ₐE₂E₃EᵢᵥE₄ | AlkLEᵥEᵥᵢ | | |
| 240 | E_{1b}E₂E₃EᵢᵥE₄ | AlkLEᵥEᵥᵢ | | |
| 241 | E₁ₐE₂ | AlkLEᵥEᵥᵢ | E₄* | |
| 242 | E_{1b}E₂ | AlkLEᵥEᵥᵢ | E₄* | |
| 243 | E₁ₐE₂E₃ | AlkLEᵥEᵥᵢ | E₄* | |
| 244 | E_{1b}E₂E₃ | AlkLEᵥEᵥᵢ | E₄* | |
| 245 | E₁ₐE₂Eᵢ | AlkLEᵥEᵥᵢ | E₄* | |
| 246 | E_{1b}E₂Eᵢ | AlkLEᵥEᵥᵢ | E₄* | |
| 247 | E₁ₐE₂Eᵢᵢ | AlkLEᵥEᵥᵢ | E₄* | |
| 248 | E_{1b}E₂Eᵢᵢ | AlkLEᵥEᵥᵢ | E₄* | |
| 249 | E₁ₐE₂EᵢᵢE_{iib} | AlkLEᵥEᵥᵢ | E₄* | |
| 250 | E_{1b}E₂EᵢᵢE_{iib} | AlkLEᵥEᵥᵢ | E₄* | |
| 251 | E₁ₐE₂E₃Eᵢ | AlkLEᵥEᵥᵢ | E₄* | |
| 252 | E_{1b}E₂E₃Eᵢ | AlkLEᵥEᵥᵢ | E₄* | |
| 253 | E₁ₐE₂E₃Eᵢᵢ | AlkLEᵥEᵥᵢ | E₄* | |
| 254 | E_{1b}E₂E₃Eᵢᵢ | AlkLEᵥEᵥᵢ | E₄* | |
| 255 | E₁ₐE₂E₃EᵢᵢE_{iib} | AlkLEᵥEᵥᵢ | E₄* | |
| 256 | E_{1b}E₂E₃EᵢᵢE_{iib} | AlkLEᵥEᵥᵢ | E₄* | |
| 257 | E₁ₐE₂E₄ | AlkLEᵥEᵥᵢ | E₄* | |
| 258 | E_{1b}E₂E₄ | AlkLEᵥEᵥᵢ | E₄* | |
| 259 | E₁ₐE₂E₃E₄ | AlkLEᵥEᵥᵢ | E₄* | |
| 260 | E_{1b}E₂E₃E₄ | AlkLEᵥEᵥᵢ | E₄* | |
| 261 | E₁ₐE₂EᵢE₄ | AlkLEᵥEᵥᵢ | E₄* | |
| 262 | E_{1b}E₂EᵢE₄ | AlkLEᵥEᵥᵢ | E₄* | |
| 263 | E₁ₐE₂EᵢᵢE₄ | AlkLEᵥEᵥᵢ | E₄* | |
| 264 | E_{1b}E₂EᵢᵢE₄ | AlkLEᵥEᵥᵢ | E₄* | |
| 265 | E₁ₐE₂EᵢᵢE_{iib}E₄ | AlkLEᵥEᵥᵢ | E₄* | |
| 266 | E_{1b}E₂EᵢᵢE_{iib}E₄ | AlkLEᵥEᵥᵢ | E₄* | |
| 267 | E₁ₐE₂E₃EᵢE₄ | AlkLEᵥEᵥᵢ | E₄* | |
| 268 | E_{1b}E₂E₃EᵢE₄ | AlkLEᵥEᵥᵢ | E₄* | |
| 269 | E₁ₐE₂E₃EᵢᵢE₄ | AlkLEᵥEᵥᵢ | E₄* | |
| 270 | E_{1b}E₂E₃EᵢᵢE₄ | AlkLEᵥEᵥᵢ | E₄* | |
| 271 | E₁ₐE₂E₃EᵢᵢE_{iib}E₄ | AlkLEᵥEᵥᵢ | E₄* | |
| 272 | E_{1b}E₂E₃EᵢᵢE_{iib}E₄ | AlkLEᵥEᵥᵢ | E₄* | |
| 273 | E₁ₐE₂Eᵢᵢᵢ | AlkLEᵥEᵥᵢ | E₄* | |
| 274 | E_{1b}E₂Eᵢᵢᵢ | AlkLEᵥEᵥᵢ | E₄* | |
| 275 | E₁ₐE₂E₃Eᵢᵢᵢ | AlkLEᵥEᵥᵢ | E₄* | |
| 276 | E_{1b}E₂E₃Eᵢᵢᵢ | AlkLEᵥEᵥᵢ | E₄* | |
| 277 | E₁ₐE₂EᵢᵢᵢE₄ | AlkLEᵥEᵥᵢ | E₄* | |
| 278 | E_{1b}E₂EᵢᵢᵢE₄ | AlkLEᵥEᵥᵢ | E₄* | |
| 279 | E₁ₐE₂E₃EᵢᵢᵢE₄ | AlkLEᵥEᵥᵢ | E₄* | |
| 280 | E_{1b}E₂E₃EᵢᵢᵢE₄ | AlkLEᵥEᵥᵢ | E₄* | |
| 281 | E₁ₐE₂Eᵢᵥ | AlkLEᵥEᵥᵢ | E₄* | |
| 282 | E_{1b}E₂Eᵢᵥ | AlkLEᵥEᵥᵢ | E₄* | |
| 283 | E₁ₐE₂E₃Eᵢᵥ | AlkLEᵥEᵥᵢ | E₄* | |
| 284 | E_{1b}E₂E₃Eᵢᵥ | AlkLEᵥEᵥᵢ | E₄* | |
| 285 | E₁ₐE₂EᵢᵥE₄ | AlkLEᵥEᵥᵢ | E₄* | |
| 286 | E_{1b}E₂EᵢᵥE₄ | AlkLEᵥEᵥᵢ | E₄* | |
| 287 | E₁ₐE₂E₃EᵢᵥE₄ | AlkLEᵥEᵥᵢ | E₄* | |
| 288 | E_{1b}E₂E₃EᵢᵥE₄ | AlkLEᵥEᵥᵢ | E₄* | |
| 289 | E₁ₐE₂ | AlkLEᵥEᵥᵢ | E₅ | |
| 290 | E_{1b}E₂ | AlkLEᵥEᵥᵢ | E₅ | |
| 291 | E₁ₐE₂E₃ | AlkLEᵥEᵥᵢ | E₅ | |
| 292 | E_{1b}E₂E₃ | AlkLEᵥEᵥᵢ | E₅ | |
| 293 | E₁ₐE₂Eᵢ | AlkLEᵥEᵥᵢ | E₅ | |
| 294 | E_{1b}E₂Eᵢ | AlkLEᵥEᵥᵢ | E₅ | |
| 295 | E₁ₐE₂Eᵢᵢ | AlkLEᵥEᵥᵢ | E₅ | |
| 296 | E_{1b}E₂Eᵢᵢ | AlkLEᵥEᵥᵢ | E₅ | |
| 297 | E₁ₐE₂EᵢᵢE_{iib} | AlkLEᵥEᵥᵢ | E₅ | |
| 298 | E_{1b}E₂EᵢᵢE_{iib} | AlkLEᵥEᵥᵢ | E₅ | |
| 299 | E₁ₐE₂E₃Eᵢ | AlkLEᵥEᵥᵢ | E₅ | |
| 300 | E_{1b}E₂E₃Eᵢ | AlkLEᵥEᵥᵢ | E₅ | |
| 301 | E₁ₐE₂E₃Eᵢᵢ | AlkLEᵥEᵥᵢ | E₅ | |
| 302 | E_{1b}E₂E₃Eᵢᵢ | AlkLEᵥEᵥᵢ | E₅ | |
| 303 | E₁ₐE₂E₃EᵢᵢE_{iib} | AlkLEᵥEᵥᵢ | E₅ | |
| 304 | E_{1b}E₂E₃EᵢᵢE_{iib} | AlkLEᵥEᵥᵢ | E₅ | |
| 305 | E₁ₐE₂E₄ | AlkLEᵥEᵥᵢ | E₅ | |
| 306 | E_{1b}E₂E₄ | AlkLEᵥEᵥᵢ | E₅ | |
| 307 | E₁ₐE₂E₃E₄ | AlkLEᵥEᵥᵢ | E₅ | |
| 308 | E_{1b}E₂E₃E₄ | AlkLEᵥEᵥᵢ | E₅ | |
| 309 | E₁ₐE₂EᵢE₄ | AlkLEᵥEᵥᵢ | E₅ | |
| 310 | E_{1b}E₂EᵢE₄ | AlkLEᵥEᵥᵢ | E₅ | |
| 311 | E₁ₐE₂EᵢᵢE₄ | AlkLEᵥEᵥᵢ | E₅ | |
| 312 | E_{1b}E₂EᵢᵢE₄ | AlkLEᵥEᵥᵢ | E₅ | |
| 313 | E₁ₐE₂EᵢᵢE_{iib}E₄ | AlkLEᵥEᵥᵢ | E₅ | |
| 314 | E_{1b}E₂EᵢᵢE_{iib}E₄ | AlkLEᵥEᵥᵢ | E₅ | |
| 315 | E₁ₐE₂E₃EᵢE₄ | AlkLEᵥEᵥᵢ | E₅ | |
| 316 | E_{1b}E₂E₃EᵢE₄ | AlkLEᵥEᵥᵢ | E₅ | |
| 317 | E₁ₐE₂E₃EᵢᵢE₄ | AlkLEᵥEᵥᵢ | E₅ | |
| 318 | E_{1b}E₂E₃EᵢᵢE₄ | AlkLEᵥEᵥᵢ | E₅ | |
| 319 | E₁ₐE₂E₃EᵢᵢE_{iib}E₄ | AlkLEᵥEᵥᵢ | E₅ | |
| 320 | E_{1b}E₂E₃EᵢᵢE_{iib}E₄ | AlkLEᵥEᵥᵢ | E₅ | |
| 321 | E₁ₐE₂Eᵢᵢᵢ | AlkLEᵥEᵥᵢ | E₅ | |
| 322 | E_{1b}E₂Eᵢᵢᵢ | AlkLEᵥEᵥᵢ | E₅ | |
| 323 | E₁ₐE₂E₃Eᵢᵢᵢ | AlkLEᵥEᵥᵢ | E₅ | |
| 324 | E_{1b}E₂E₃Eᵢᵢᵢ | AlkLEᵥEᵥᵢ | E₅ | |
| 325 | E₁ₐE₂EᵢᵢᵢE₄ | AlkLEᵥEᵥᵢ | E₅ | |
| 326 | E_{1b}E₂EᵢᵢᵢE₄ | AlkLEᵥEᵥᵢ | E₅ | |
| 327 | E₁ₐE₂E₃EᵢᵢᵢE₄ | AlkLEᵥEᵥᵢ | E₅ | |
| 328 | E_{1b}E₂E₃EᵢᵢᵢE₄ | AlkLEᵥEᵥᵢ | E₅ | |
| 329 | E₁ₐE₂Eᵢᵥ | AlkLEᵥEᵥᵢ | E₅ | |
| 330 | E_{1b}E₂Eᵢᵥ | AlkLEᵥEᵥᵢ | E₅ | |
| 331 | E₁ₐE₂E₃Eᵢᵥ | AlkLEᵥEᵥᵢ | E₅ | |
| 332 | E_{1b}E₂E₃Eᵢᵥ | AlkLEᵥEᵥᵢ | E₅ | |
| 333 | E₁ₐE₂EᵢᵥE₄ | AlkLEᵥEᵥᵢ | E₅ | |
| 334 | E_{1b}E₂EᵢᵥE₄ | AlkLEᵥEᵥᵢ | E₅ | |
| 335 | E₁ₐE₂E₃EᵢᵥE₄ | AlkLEᵥEᵥᵢ | E₅ | |
| 336 | E_{1b}E₂E₃EᵢᵥE₄ | AlkLEᵥEᵥᵢ | E₅ | |
| 337 | E₁ₐE₂ | AlkLEᵥEᵥᵢ | E₄*E₅ | |
| 338 | E_{1b}E₂ | AlkLEᵥEᵥᵢ | E₄*E₅ | |
| 339 | E₁ₐE₂E₃ | AlkLEᵥEᵥᵢ | E₄*E₅ | |
| 340 | E_{1b}E₂E₃ | AlkLEᵥEᵥᵢ | E₄*E₅ | |
| 341 | E₁ₐE₂Eᵢ | AlkLEᵥEᵥᵢ | E₄*E₅ | |
| 342 | E_{1b}E₂Eᵢ | AlkLEᵥEᵥᵢ | E₄*E₅ | |
| 343 | E₁ₐE₂Eᵢᵢ | AlkLEᵥEᵥᵢ | E₄*E₅ | |
| 344 | E_{1b}E₂Eᵢᵢ | AlkLEᵥEᵥᵢ | E₄*E₅ | |
| 345 | E₁ₐE₂EᵢᵢE_{iib} | AlkLEᵥEᵥᵢ | E₄*E₅ | |
| 346 | E_{1b}E₂EᵢᵢE_{iib} | AlkLEᵥEᵥᵢ | E₄*E₅ | |
| 347 | E₁ₐE₂E₃Eᵢ | AlkLEᵥEᵥᵢ | E₄*E₅ | |
| 348 | E_{1b}E₂E₃Eᵢ | AlkLEᵥEᵥᵢ | E₄*E₅ | |
| 349 | E₁ₐE₂E₃Eᵢᵢ | AlkLEᵥEᵥᵢ | E₄*E₅ | |
| 350 | E_{1b}E₂E₃Eᵢᵢ | AlkLEᵥEᵥᵢ | E₄*E₅ | |
| 351 | E₁ₐE₂E₃EᵢᵢE_{iib} | AlkLEᵥEᵥᵢ | E₄*E₅ | |
| 352 | E_{1b}E₂E₃EᵢᵢE_{iib} | AlkLEᵥEᵥᵢ | E₄*E₅ | |
| 353 | E₁ₐE₂E₄ | AlkLEᵥEᵥᵢ | E₄*E₅ | |
| 354 | E_{1b}E₂E₄ | AlkLEᵥEᵥᵢ | E₄*E₅ | |
| 355 | E₁ₐE₂E₃E₄ | AlkLEᵥEᵥᵢ | E₄*E₅ | |
| 356 | E_{1b}E₂E₃E₄ | AlkLEᵥEᵥᵢ | E₄*E₅ | |
| 357 | E₁ₐE₂EᵢE₄ | AlkLEᵥEᵥᵢ | E₄*E₅ | |
| 358 | E_{1b}E₂EᵢE₄ | AlkLEᵥEᵥᵢ | E₄*E₅ | |
| 359 | E₁ₐE₂EᵢᵢE₄ | AlkLEᵥEᵥᵢ | E₄*E₅ | |
| 360 | E_{1b}E₂EᵢᵢE₄ | AlkLEᵥEᵥᵢ | E₄*E₅ | |
| 361 | E₁ₐE₂EᵢᵢE_{iib}E₄ | AlkLEᵥEᵥᵢ | E₄*E₅ | |
| 362 | E_{1b}E₂EᵢᵢE_{iib}E₄ | AlkLEᵥEᵥᵢ | E₄*E₅ | |
| 363 | E₁ₐE₂E₃EᵢE₄ | AlkLEᵥEᵥᵢ | E₄*E₅ | |
| 364 | E_{1b}E₂E₃EᵢE₄ | AlkLEᵥEᵥᵢ | E₄*E₅ | |
| 365 | E₁ₐE₂E₃EᵢᵢE₄ | AlkLEᵥEᵥᵢ | E₄*E₅ | |
| 366 | E_{1b}E₂E₃EᵢᵢE₄ | AlkLEᵥEᵥᵢ | E₄*E₅ | |
| 367 | E₁ₐE₂E₃EᵢᵢE_{iib}E₄ | AlkLEᵥEᵥᵢ | E₄*E₅ | |
| 368 | E_{1b}E₂E₃EᵢᵢE_{iib}E₄ | AlkLEᵥEᵥᵢ | E₄*E₅ | |
| 369 | E₁ₐE₂Eᵢᵢᵢ | AlkLEᵥEᵥᵢ | E₄*E₅ | |
| 370 | E_{1b}E₂Eᵢᵢᵢ | AlkLEᵥEᵥᵢ | E₄*E₅ | |
| 371 | E₁ₐE₂E₃Eᵢᵢᵢ | AlkLEᵥEᵥᵢ | E₄*E₅ | |
| 372 | E_{1b}E₂E₃Eᵢᵢᵢ | AlkLEᵥEᵥᵢ | E₄*E₅ | |
| 373 | E₁ₐE₂EᵢᵢᵢE₄ | AlkLEᵥEᵥᵢ | E₄*E₅ | |
| 374 | E_{1b}E₂EᵢᵢᵢE₄ | AlkLEᵥEᵥᵢ | E₄*E₅ | |
| 375 | E₁ₐE₂E₃EᵢᵢᵢE₄ | AlkLEᵥEᵥᵢ | E₄*E₅ | |
| 376 | E_{1b}E₂E₃EᵢᵢᵢE₄ | AlkLEᵥEᵥᵢ | E₄*E₅ | |
| 377 | E₁ₐE₂Eᵢᵥ | AlkLEᵥEᵥᵢ | E₄*E₅ | |
| 378 | E_{1b}E₂Eᵢᵥ | AlkLEᵥEᵥᵢ | E₄*E₅ | |
| 379 | E₁ₐE₂E₃Eᵢᵥ | AlkLEᵥEᵥᵢ | E₄*E₅ | |
| 380 | E_{1b}E₂E₃Eᵢᵥ | AlkLEᵥEᵥᵢ | E₄*E₅ | |
| 381 | E₁ₐE₂EᵢᵥE₄ | AlkLEᵥEᵥᵢ | E₄*E₅ | |
| 382 | E_{1b}E₂EᵢᵥE₄ | AlkLEᵥEᵥᵢ | E₄*E₅ | |
| 383 | E₁ₐE₂E₃EᵢᵥE₄ | AlkLEᵥEᵥᵢ | E₄*E₅ | |
| 384 | E_{1b}E₂E₃EᵢᵥE₄ | AlkLEᵥEᵥᵢ | E₄*E₅ | |
| 385 | E₁ₐE₂ | EᵥEᵥᵢ | | Eₐ |
| 386 | E_{1b}E₂ | EᵥEᵥᵢ | | Eₐ |
| 387 | E₁ₐE₂E₃ | EᵥEᵥᵢ | | Eₐ |
| 388 | E_{1b}E₂E₃ | EᵥEᵥᵢ | | Eₐ |
| 389 | E₁ₐE₂Eᵢ | EᵥEᵥᵢ | | Eₐ |
| 390 | E_{1b}E₂Eᵢ | EᵥEᵥᵢ | | Eₐ |
| 391 | E₁ₐE₂Eᵢᵢ | EᵥEᵥᵢ | | Eₐ |
| 392 | E_{1b}E₂Eᵢᵢ | EᵥEᵥᵢ | | Eₐ |
| 393 | E₁ₐE₂EᵢᵢE_{iib} | EᵥEᵥᵢ | | Eₐ |
| 394 | E_{1b}E₂EᵢᵢE_{iib} | EᵥEᵥᵢ | | Eₐ |
| 395 | E₁ₐE₂E₃Eᵢ | EᵥEᵥᵢ | | Eₐ |
| 396 | E_{1b}E₂E₃Eᵢ | EᵥEᵥᵢ | | Eₐ |
| 397 | E₁ₐE₂E₃Eᵢᵢ | EᵥEᵥᵢ | | Eₐ |
| 398 | E_{1b}E₂E₃Eᵢᵢ | EᵥEᵥᵢ | | Eₐ |
| 399 | E₁ₐE₂E₃EᵢᵢE_{iib} | EᵥEᵥᵢ | | Eₐ |
| 400 | E_{1b}E₂E₃EᵢᵢE_{iib} | EᵥEᵥᵢ | | Eₐ |
| 401 | E₁ₐE₂E₄ | EᵥEᵥᵢ | | Eₐ |
| 402 | E_{1b}E₂E₄ | EᵥEᵥᵢ | | Eₐ |
| 403 | E₁ₐE₂E₃E₄ | EᵥEᵥᵢ | | Eₐ |
| 404 | E_{1b}E₂E₃E₄ | EᵥEᵥᵢ | | Eₐ |
| 405 | E₁ₐE₂EᵢE₄ | EᵥEᵥᵢ | | Eₐ |
| 406 | E_{1b}E₂EᵢE₄ | EᵥEᵥᵢ | | Eₐ |
| 407 | E₁ₐE₂EᵢᵢE₄ | EᵥEᵥᵢ | | Eₐ |
| 408 | E_{1b}E₂EᵢᵢE₄ | EᵥEᵥᵢ | | Eₐ |
| 409 | E₁ₐE₂EᵢᵢE_{iib}E₄ | EᵥEᵥᵢ | | Eₐ |
| 410 | E_{1b}E₂EᵢᵢE_{iib}E₄ | EᵥEᵥᵢ | | Eₐ |
| 411 | E₁ₐE₂E₃EᵢE₄ | EᵥEᵥᵢ | | Eₐ |
| 412 | E_{1b}E₂E₃EᵢE₄ | EᵥEᵥᵢ | | Eₐ |
| 413 | E₁ₐE₂E₃EᵢᵢE₄ | EᵥEᵥᵢ | | Eₐ |
| 414 | E_{1b}E₂E₃EᵢᵢE₄ | EᵥEᵥᵢ | | Eₐ |
| 415 | E₁ₐE₂E₃EᵢᵢE_{iib}E₄ | EᵥEᵥᵢ | | Eₐ |
| 416 | E_{1b}E₂E₃EᵢᵢE_{iib}E₄ | EᵥEᵥᵢ | | Eₐ |
| 417 | E₁ₐE₂Eᵢᵢᵢ | EᵥEᵥᵢ | | Eₐ |
| 418 | E_{1b}E₂Eᵢᵢᵢ | EᵥEᵥᵢ | | Eₐ |
| 419 | E₁ₐE₂E₃Eᵢᵢᵢ | EᵥEᵥᵢ | | Eₐ |
| 420 | E_{1b}E₂E₃Eᵢᵢᵢ | EᵥEᵥᵢ | | Eₐ |
| 421 | E₁ₐE₂EᵢᵢᵢE₄ | EᵥEᵥᵢ | | Eₐ |
| 422 | E_{1b}E₂EᵢᵢᵢE₄ | EᵥEᵥᵢ | | Eₐ |
| 423 | E₁ₐE₂E₃EᵢᵢᵢE₄ | EᵥEᵥᵢ | | Eₐ |
| 424 | E_{1b}E₂E₃EᵢᵢᵢE₄ | EᵥEᵥᵢ | | Eₐ |
| 425 | E₁ₐE₂Eᵢᵥ | EᵥEᵥᵢ | | Eₐ |
| 426 | E_{1b}E₂Eᵢᵥ | EᵥEᵥᵢ | | Eₐ |
| 427 | E₁ₐE₂E₃Eᵢᵥ | EᵥEᵥᵢ | | Eₐ |
| 428 | E_{1b}E₂E₃Eᵢᵥ | EᵥEᵥᵢ | | Eₐ |
| 429 | E₁ₐE₂EᵢᵥE₄ | EᵥEᵥᵢ | | Eₐ |
| 430 | E_{1b}E₂EᵢᵥE₄ | EᵥEᵥᵢ | | Eₐ |
| 431 | E₁ₐE₂E₃EᵢᵥE₄ | EᵥEᵥᵢ | | Eₐ |
| 432 | E_{1b}E₂E₃EᵢᵥE₄ | EᵥEᵥᵢ | | Eₐ |
| 433 | E₁ₐE₂ | EᵥEᵥᵢ | E₄* | Eₐ |
| 434 | E_{1b}E₂ | EᵥEᵥᵢ | E₄* | Eₐ |
| 435 | E₁ₐE₂E₃ | EᵥEᵥᵢ | E₄* | Eₐ |
| 436 | E_{1b}E₂E₃ | EᵥEᵥᵢ | E₄* | Eₐ |
| 437 | E₁ₐE₂Eᵢ | EᵥEᵥᵢ | E₄* | Eₐ |
| 438 | E_{1b}E₂Eᵢ | EᵥEᵥᵢ | E₄* | Eₐ |
| 439 | E₁ₐE₂Eᵢᵢ | EᵥEᵥᵢ | E₄* | Eₐ |
| 440 | E_{1b}E₂Eᵢᵢ | EᵥEᵥᵢ | E₄* | Eₐ |
| 441 | E₁ₐE₂EᵢᵢE_{iib} | EᵥEᵥᵢ | E₄* | Eₐ |
| 442 | E_{1b}E₂EᵢᵢE_{iib} | EᵥEᵥᵢ | E₄* | Eₐ |
| 443 | E₁ₐE₂E₃Eᵢ | EᵥEᵥᵢ | E₄* | Eₐ |
| 444 | E_{1b}E₂E₃Eᵢ | EᵥEᵥᵢ | E₄* | Eₐ |
| 445 | E₁ₐE₂E₃Eᵢᵢ | EᵥEᵥᵢ | E₄* | Eₐ |
| 446 | E_{1b}E₂E₃Eᵢᵢ | EᵥEᵥᵢ | E₄* | Eₐ |
| 447 | E₁ₐE₂E₃EᵢᵢE_{iib} | EᵥEᵥᵢ | E₄* | Eₐ |
| 448 | E_{1b}E₂E₃EᵢᵢE_{iib} | EᵥEᵥᵢ | E₄* | Eₐ |
| 449 | E₁ₐE₂E₄ | EᵥEᵥᵢ | E₄* | Eₐ |
| 450 | E_{1b}E₂E₄ | EᵥEᵥᵢ | E₄* | Eₐ |
| 451 | E₁ₐE₂E₃E₄ | EᵥEᵥᵢ | E₄* | Eₐ |
| 452 | E_{1b}E₂E₃E₄ | EᵥEᵥᵢ | E₄* | Eₐ |
| 453 | E₁ₐE₂EᵢE₄ | EᵥEᵥᵢ | E₄* | Eₐ |
| 454 | E_{1b}E₂EᵢE₄ | EᵥEᵥᵢ | E₄* | Eₐ |
| 455 | E₁ₐE₂EᵢᵢE₄ | EᵥEᵥᵢ | E₄* | Eₐ |
| 456 | E_{1b}E₂EᵢᵢE₄ | EᵥEᵥᵢ | E₄* | Eₐ |
| 457 | E₁ₐE₂EᵢᵢE_{iib}E₄ | EᵥEᵥᵢ | E₄* | Eₐ |
| 458 | E_{1b}E₂EᵢᵢE_{iib}E₄ | EᵥEᵥᵢ | E₄* | Eₐ |
| 459 | E₁ₐE₂E₃EᵢE₄ | EᵥEᵥᵢ | E₄* | Eₐ |
| 460 | E_{1b}E₂E₃EᵢE₄ | EᵥEᵥᵢ | E₄* | Eₐ |
| 461 | E₁ₐE₂E₃EᵢᵢE₄ | EᵥEᵥᵢ | E₄* | Eₐ |
| 462 | E_{1b}E₂E₃EᵢᵢE₄ | EᵥEᵥᵢ | E₄* | Eₐ |
| 463 | E₁ₐE₂E₃EᵢᵢE_{iib}E₄ | EᵥEᵥᵢ | E₄* | Eₐ |
| 464 | E_{1b}E₂E₃EᵢᵢE_{iib}E₄ | EᵥEᵥᵢ | E₄* | Eₐ |
| 465 | E₁ₐE₂Eᵢᵢᵢ | EᵥEᵥᵢ | E₄* | Eₐ |
| 466 | E_{1b}E₂Eᵢᵢᵢ | EᵥEᵥᵢ | E₄* | Eₐ |
| 467 | E₁ₐE₂E₃Eᵢᵢᵢ | EᵥEᵥᵢ | E₄* | Eₐ |
| 468 | E_{1b}E₂E₃Eᵢᵢᵢ | EᵥEᵥᵢ | E₄* | Eₐ |
| 469 | E₁ₐE₂EᵢᵢᵢE₄ | EᵥEᵥᵢ | E₄* | Eₐ |
| 470 | E_{1b}E₂EᵢᵢᵢE₄ | EᵥEᵥᵢ | E₄* | Eₐ |
| 471 | E₁ₐE₂E₃EᵢᵢᵢE₄ | EᵥEᵥᵢ | E₄* | Eₐ |
| 472 | E_{1b}E₂E₃EᵢᵢᵢE₄ | EᵥEᵥᵢ | E₄* | Eₐ |
| 473 | E₁ₐE₂Eᵢᵥ | EᵥEᵥᵢ | E₄* | Eₐ |
| 474 | E_{1b}E₂Eᵢᵥ | EᵥEᵥᵢ | E₄* | Eₐ |
| 475 | E₁ₐE₂E₃Eᵢᵥ | EᵥEᵥᵢ | E₄* | Eₐ |
| 476 | E_{1b}E₂E₃Eᵢᵥ | EᵥEᵥᵢ | E₄* | Eₐ |
| 477 | E₁ₐE₂EᵢᵥE₄ | EᵥEᵥᵢ | E₄* | Eₐ |
| 478 | E_{1b}E₂EᵢᵥE₄ | EᵥEᵥᵢ | E₄* | Eₐ |
| 479 | E₁ₐE₂E₃EᵢᵥE₄ | EᵥEᵥᵢ | E₄* | Eₐ |
| 480 | E_{1b}E₂E₃EᵢᵥE₄ | EᵥEᵥᵢ | E₄* | Eₐ |
| 481 | E₁ₐE₂ | EᵥEᵥᵢ | E₅ | Eₐ |
| 482 | E_{1b}E₂ | EᵥEᵥᵢ | E₅ | Eₐ |
| 483 | E₁ₐE₂E₃ | EᵥEᵥᵢ | E₅ | Eₐ |
| 484 | E_{1b}E₂E₃ | EᵥEᵥᵢ | E₅ | Eₐ |
| 485 | E₁ₐE₂Eᵢ | EᵥEᵥᵢ | E₅ | Eₐ |
| 486 | E_{1b}E₂Eᵢ | EᵥEᵥᵢ | E₅ | Eₐ |
| 487 | E₁ₐE₂Eᵢᵢ | EᵥEᵥᵢ | E₅ | Eₐ |
| 488 | E_{1b}E₂Eᵢᵢ | EᵥEᵥᵢ | E₅ | Eₐ |
| 489 | E₁ₐE₂EᵢᵢE_{iib} | EᵥEᵥᵢ | E₅ | Eₐ |
| 490 | E_{1b}E₂EᵢᵢE_{iib} | EᵥEᵥᵢ | E₅ | Eₐ |
| 491 | E₁ₐE₂E₃Eᵢ | EᵥEᵥᵢ | E₅ | Eₐ |
| 492 | E_{1b}E₂E₃Eᵢ | EᵥEᵥᵢ | E₅ | Eₐ |
| 493 | E₁ₐE₂E₃Eᵢᵢ | EᵥEᵥᵢ | E₅ | Eₐ |
| 494 | E_{1b}E₂E₃Eᵢᵢ | EᵥEᵥᵢ | E₅ | Eₐ |
| 495 | E₁ₐE₂E₃EᵢᵢE_{iib} | EᵥEᵥᵢ | E₅ | Eₐ |
| 496 | E_{1b}E₂E₃EᵢᵢE_{iib} | EᵥEᵥᵢ | E₅ | Eₐ |
| 497 | E₁ₐE₂E₄ | EᵥEᵥᵢ | E₅ | Eₐ |
| 498 | E_{1b}E₂E₄ | EᵥEᵥᵢ | E₅ | Eₐ |
| 499 | E₁ₐE₂E₃E₄ | EᵥEᵥᵢ | E₅ | Eₐ |
| 500 | E_{1b}E₂E₃E₄ | EᵥEᵥᵢ | E₅ | Eₐ |
| 501 | E₁ₐE₂EᵢE₄ | EᵥEᵥᵢ | E₅ | Eₐ |
| 502 | E_{1b}E₂EᵢE₄ | EᵥEᵥᵢ | E₅ | Eₐ |
| 503 | E₁ₐE₂EᵢᵢE₄ | EᵥEᵥᵢ | E₅ | Eₐ |
| 504 | E_{1b}E₂EᵢᵢE₄ | EᵥEᵥᵢ | E₅ | Eₐ |
| 505 | E₁ₐE₂EᵢᵢE_{iib}E₄ | EᵥEᵥᵢ | E₅ | Eₐ |
| 506 | E_{1b}E₂EᵢᵢE_{iib}E₄ | EᵥEᵥᵢ | E₅ | Eₐ |
| 507 | E₁ₐE₂E₃EᵢE₄ | EᵥEᵥᵢ | E₅ | Eₐ |
| 508 | E_{1b}E₂E₃EᵢE₄ | EᵥEᵥᵢ | E₅ | Eₐ |
| 509 | E₁ₐE₂E₃EᵢᵢE₄ | EᵥEᵥᵢ | E₅ | Eₐ |
| 510 | E_{1b}E₂E₃EᵢᵢE₄ | EᵥEᵥᵢ | E₅ | Eₐ |
| 511 | E₁ₐE₂E₃EᵢᵢE_{iib}E₄ | EᵥEᵥᵢ | E₅ | Eₐ |
| 512 | E_{1b}E₂E₃EᵢᵢE_{iib}E₄ | EᵥEᵥᵢ | E₅ | Eₐ |
| 513 | E₁ₐE₂Eᵢᵢᵢ | EᵥEᵥᵢ | E₅ | Eₐ |
| 514 | E_{1b}E₂Eᵢᵢᵢ | EᵥEᵥᵢ | E₅ | Eₐ |
| 515 | E₁ₐE₂E₃Eᵢᵢᵢ | EᵥEᵥᵢ | E₅ | Eₐ |
| 516 | E_{1b}E₂E₃Eᵢᵢᵢ | EᵥEᵥᵢ | E₅ | Eₐ |
| 517 | E₁ₐE₂EᵢᵢᵢE₄ | EᵥEᵥᵢ | E₅ | Eₐ |
| 518 | E_{1b}E₂EᵢᵢᵢE₄ | EᵥEᵥᵢ | E₅ | Eₐ |
| 519 | E₁ₐE₂E₃EᵢᵢᵢE₄ | EᵥEᵥᵢ | E₅ | Eₐ |
| 520 | E_{1b}E₂E₃EᵢᵢᵢE₄ | EᵥEᵥᵢ | E₅ | Eₐ |
| 521 | E₁ₐE₂Eᵢᵥ | EᵥEᵥᵢ | E₅ | Eₐ |
| 522 | E_{1b}E₂Eᵢᵥ | EᵥEᵥᵢ | E₅ | Eₐ |
| 523 | E₁ₐE₂E₃Eᵢᵥ | EᵥEᵥᵢ | E₅ | Eₐ |
| 524 | E_{1b}E₂E₃Eᵢᵥ | EᵥEᵥᵢ | E₅ | Eₐ |
| 525 | E₁ₐE₂EᵢᵥE₄ | EᵥEᵥᵢ | E₅ | Eₐ |
| 526 | E_{1b}E₂EᵢᵥE₄ | EᵥEᵥᵢ | E₅ | Eₐ |
| 527 | E₁ₐE₂E₃EᵢᵥE₄ | EᵥEᵥᵢ | E₅ | Eₐ |
| 528 | E_{1b}E₂E₃EᵢᵥE₄ | EᵥEᵥᵢ | E₅ | Eₐ |
| 529 | E₁ₐE₂ | EᵥEᵥᵢ | E₄*E₅ | Eₐ |
| 530 | E_{1b}E₂ | EᵥEᵥᵢ | E₄*E₅ | Eₐ |
| 531 | E₁ₐE₂E₃ | EᵥEᵥᵢ | E₄*E₅ | Eₐ |
| 532 | E_{1b}E₂E₃ | EᵥEᵥᵢ | E₄*E₅ | Eₐ |
| 533 | E₁ₐE₂Eᵢ | EᵥEᵥᵢ | E₄*E₅ | Eₐ |
| 534 | E_{1b}E₂Eᵢ | EᵥEᵥᵢ | E₄*E₅ | Eₐ |
| 535 | E₁ₐE₂Eᵢᵢ | EᵥEᵥᵢ | E₄*E₅ | Eₐ |
| 536 | E_{1b}E₂Eᵢᵢ | EᵥEᵥᵢ | E₄*E₅ | Eₐ |
| 537 | E₁ₐE₂EᵢᵢE_{iib} | EᵥEᵥᵢ | E₄*E₅ | Eₐ |
| 538 | E_{1b}E₂EᵢᵢE_{iib} | EᵥEᵥᵢ | E₄*E₅ | Eₐ |
| 539 | E₁ₐE₂E₃Eᵢ | EᵥEᵥᵢ | E₄*E₅ | Eₐ |
| 540 | E_{1b}E₂E₃Eᵢ | EᵥEᵥᵢ | E₄*E₅ | Eₐ |
| 541 | E₁ₐE₂E₃Eᵢᵢ | EᵥEᵥᵢ | E₄*E₅ | Eₐ |
| 542 | E_{1b}E₂E₃Eᵢᵢ | EᵥEᵥᵢ | E₄*E₅ | Eₐ |
| 543 | E₁ₐE₂E₃EᵢᵢE_{iib} | EᵥEᵥᵢ | E₄*E₅ | Eₐ |
| 544 | E_{1b}E₂E₃EᵢᵢE_{iib} | EᵥEᵥᵢ | E₄*E₅ | Eₐ |
| 545 | E₁ₐE₂E₄ | EᵥEᵥᵢ | E₄*E₅ | Eₐ |
| 546 | E_{1b}E₂E₄ | EᵥEᵥᵢ | E₄*E₅ | Eₐ |
| 547 | E₁ₐE₂E₃E₄ | EᵥEᵥᵢ | E₄*E₅ | Eₐ |
| 548 | E_{1b}E₂E₃E₄ | EᵥEᵥᵢ | E₄*E₅ | Eₐ |
| 549 | E₁ₐE₂EᵢE₄ | EᵥEᵥᵢ | E₄*E₅ | Eₐ |
| 550 | E_{1b}E₂EᵢE₄ | EᵥEᵥᵢ | E₄*E₅ | Eₐ |
| 551 | E₁ₐE₂EᵢᵢE₄ | EᵥEᵥᵢ | E₄*E₅ | Eₐ |
| 552 | E_{1b}E₂EᵢᵢE₄ | EᵥEᵥᵢ | E₄*E₅ | Eₐ |
| 553 | E₁ₐE₂EᵢᵢE_{iib}E₄ | EᵥEᵥᵢ | E₄*E₅ | Eₐ |
| 554 | E_{1b}E₂EᵢᵢE_{iib}E₄ | EᵥEᵥᵢ | E₄*E₅ | Eₐ |
| 555 | E₁ₐE₂E₃EᵢE₄ | EᵥEᵥᵢ | E₄*E₅ | Eₐ |
| 556 | E_{1b}E₂E₃EᵢE₄ | EᵥEᵥᵢ | E₄*E₅ | Eₐ |
| 557 | E₁ₐE₂E₃EᵢᵢE₄ | EᵥEᵥᵢ | E₄*E₅ | Eₐ |
| 558 | E_{1b}E₂E₃EᵢᵢE₄ | EᵥEᵥᵢ | E₄*E₅ | Eₐ |
| 559 | E₁ₐE₂E₃EᵢᵢE_{iib}E₄ | EᵥEᵥᵢ | E₄*E₅ | Eₐ |
| 560 | E_{1b}E₂E₃EᵢᵢE_{iib}E₄ | EᵥEᵥᵢ | E₄*E₅ | Eₐ |
| 561 | E₁ₐE₂Eᵢᵢᵢ | EᵥEᵥᵢ | E₄*E₅ | Eₐ |
| 562 | E_{1b}E₂Eᵢᵢᵢ | EᵥEᵥᵢ | E₄*E₅ | Eₐ |
| 563 | E₁ₐE₂E₃Eᵢᵢᵢ | EᵥEᵥᵢ | E₄*E₅ | Eₐ |
| 564 | E_{1b}E₂E₃Eᵢᵢᵢ | EᵥEᵥᵢ | E₄*E₅ | Eₐ |
| 565 | E₁ₐE₂EᵢᵢᵢE₄ | EᵥEᵥᵢ | E₄*E₅ | Eₐ |
| 566 | E_{1b}E₂EᵢᵢᵢE₄ | EᵥEᵥᵢ | E₄*E₅ | Eₐ |
| 567 | E₁ₐE₂E₃EᵢᵢᵢE₄ | EᵥEᵥᵢ | E₄*E₅ | Eₐ |
| 568 | E_{1b}E₂E₃EᵢᵢᵢE₄ | EᵥEᵥᵢ | E₄*E₅ | Eₐ |
| 569 | E₁ₐE₂Eᵢᵥ | EᵥEᵥᵢ | E₄*E₅ | Eₐ |
| 570 | E_{1b}E₂Eᵢᵥ | EᵥEᵥᵢ | E₄*E₅ | Eₐ |
| 571 | E₁ₐE₂E₃Eᵢᵥ | EᵥEᵥᵢ | E₄*E₅ | Eₐ |
| 572 | E_{1b}E₂E₃Eᵢᵥ | EᵥEᵥᵢ | E₄*E₅ | Eₐ |
| 573 | E₁ₐE₂EᵢᵥE₄ | EᵥEᵥᵢ | E₄*E₅ | Eₐ |
| 574 | E_{1b}E₂EᵢᵥE₄ | EᵥEᵥᵢ | E₄*E₅ | Eₐ |
| 575 | E₁ₐE₂E₃EᵢᵥE₄ | EᵥEᵥᵢ | E₄*E₅ | Eₐ |
| 576 | E_{1b}E₂E₃EᵢᵥE₄ | EᵥEᵥᵢ | E₄*E₅ | Eₐ |
| 577 | E₁ₐE₂ | AlkLEᵥEᵥᵢ | | Eₐ |
| 578 | E_{1b}E₂ | AlkLEᵥEᵥᵢ | | Eₐ |
| 579 | E₁ₐE₂E₃ | AlkLEᵥEᵥᵢ | | Eₐ |
| 580 | E_{1b}E₂E₃ | AlkLEᵥEᵥᵢ | | Eₐ |
| 581 | E₁ₐE₂Eᵢ | AlkLEᵥEᵥᵢ | | Eₐ |
| 582 | E_{1b}E₂Eᵢ | AlkLEᵥEᵥᵢ | | Eₐ |
| 583 | E₁ₐE₂Eᵢᵢ | AlkLEᵥEᵥᵢ | | Eₐ |
| 584 | E_{1b}E₂Eᵢᵢ | AlkLEᵥEᵥᵢ | | Eₐ |
| 585 | E₁ₐE₂EᵢᵢE_{iib} | AlkLEᵥEᵥᵢ | | Eₐ |
| 586 | E_{1b}E₂EᵢᵢE_{iib} | AlkLEᵥEᵥᵢ | | Eₐ |
| 587 | E₁ₐE₂E₃Eᵢ | AlkLEᵥEᵥᵢ | | Eₐ |
| 588 | E_{1b}E₂E₃Eᵢ | AlkLEᵥEᵥᵢ | | Eₐ |
| 589 | E₁ₐE₂E₃Eᵢᵢ | AlkLEᵥEᵥᵢ | | Eₐ |
| 590 | E_{1b}E₂E₃Eᵢᵢ | AlkLEᵥEᵥᵢ | | Eₐ |
| 591 | E₁ₐE₂E₃EᵢᵢE_{iib} | AlkLEᵥEᵥᵢ | | Eₐ |
| 592 | E_{1b}E₂E₃EᵢᵢE_{iib} | AlkLEᵥEᵥᵢ | | Eₐ |
| 593 | E₁ₐE₂E₄ | AlkLEᵥEᵥᵢ | | Eₐ |
| 594 | E_{1b}E₂E₄ | AlkLEᵥEᵥᵢ | | Eₐ |
| 595 | E₁ₐE₂E₃E₄ | AlkLEᵥEᵥᵢ | | Eₐ |
| 596 | E_{1b}E₂E₃E₄ | AlkLEᵥEᵥᵢ | | Eₐ |
| 597 | E₁ₐE₂EᵢE₄ | AlkLEᵥEᵥᵢ | | Eₐ |
| 598 | E_{1b}E₂EᵢE₄ | AlkLEᵥEᵥᵢ | | Eₐ |
| 599 | E₁ₐE₂EᵢᵢE₄ | AlkLEᵥEᵥᵢ | | Eₐ |
| 600 | E_{1b}E₂EᵢᵢE₄ | AlkLEᵥEᵥᵢ | | Eₐ |
| 601 | E₁ₐE₂EᵢᵢE_{iib}E₄ | AlkLEᵥEᵥᵢ | | Eₐ |
| 602 | E_{1b}E₂EᵢᵢE_{iib}E₄ | AlkLEᵥEᵥᵢ | | Eₐ |
| 603 | E₁ₐE₂E₃EᵢE₄ | AlkLEᵥEᵥᵢ | | Eₐ |
| 604 | E_{1b}E₂E₃EᵢE₄ | AlkLEᵥEᵥᵢ | | Eₐ |
| 605 | E₁ₐE₂E₃EᵢᵢE₄ | AlkLEᵥEᵥᵢ | | Eₐ |
| 606 | E_{1b}E₂E₃EᵢᵢE₄ | AlkLEᵥEᵥᵢ | | Eₐ |
| 607 | E₁ₐE₂E₃EᵢᵢE_{iib}E₄ | AlkLEᵥEᵥᵢ | | Eₐ |
| 608 | E_{1b}E₂E₃EᵢᵢE_{iib}E₄ | AlkLEᵥEᵥᵢ | | Eₐ |
| 609 | E₁ₐE₂Eᵢᵢᵢ | AlkLEᵥEᵥᵢ | | Eₐ |
| 610 | E_{1b}E₂Eᵢᵢᵢ | AlkLEᵥEᵥᵢ | | Eₐ |
| 611 | E₁ₐE₂E₃Eᵢᵢᵢ | AlkLEᵥEᵥᵢ | | Eₐ |
| 612 | E_{1b}E₂E₃Eᵢᵢᵢ | AlkLEᵥEᵥᵢ | | Eₐ |
| 613 | E₁ₐE₂EᵢᵢᵢE₄ | AlkLEᵥEᵥᵢ | | Eₐ |
| 614 | E_{1b}E₂EᵢᵢᵢE₄ | AlkLEᵥEᵥᵢ | | Eₐ |
| 615 | E₁ₐE₂E₃EᵢᵢᵢE₄ | AlkLEᵥEᵥᵢ | | Eₐ |
| 616 | E_{1b}E₂E₃EᵢᵢᵢE₄ | AlkLEᵥEᵥᵢ | | Eₐ |
| 617 | E₁ₐE₂Eᵢᵥ | AlkLEᵥEᵥᵢ | | Eₐ |
| 618 | E_{1b}E₂Eᵢᵥ | AlkLEᵥEᵥᵢ | | Eₐ |
| 619 | E₁ₐE₂E₃Eᵢᵥ | AlkLEᵥEᵥᵢ | | Eₐ |
| 620 | E_{1b}E₂E₃Eᵢᵥ | AlkLEᵥEᵥᵢ | | Eₐ |
| 621 | E₁ₐE₂EᵢᵥE₄ | AlkLEᵥEᵥᵢ | | Eₐ |
| 622 | E_{1b}E₂EᵢᵥE₄ | AlkLEᵥEᵥᵢ | | Eₐ |
| 623 | E₁ₐE₂E₃EᵢᵥE₄ | AlkLEᵥEᵥᵢ | | Eₐ |
| 624 | E_{1b}E₂E₃EᵢᵥE₄ | AlkLEᵥEᵥᵢ | | Eₐ |
| 625 | E₁ₐE₂ | AlkLEᵥEᵥᵢ | E₄* | Eₐ |
| 626 | E_{1b}E₂ | AlkLEᵥEᵥᵢ | E₄* | Eₐ |
| 627 | E₁ₐE₂E₃ | AlkLEᵥEᵥᵢ | E₄* | Eₐ |
| 628 | E_{1b}E₂E₃ | AlkLEᵥEᵥᵢ | E₄* | Eₐ |
| 629 | E₁ₐE₂Eᵢ | AlkLEᵥEᵥᵢ | E₄* | Eₐ |
| 630 | E_{1b}E₂Eᵢ | AlkLEᵥEᵥᵢ | E₄* | Eₐ |
| 631 | E₁ₐE₂Eᵢᵢ | AlkLEᵥEᵥᵢ | E₄* | Eₐ |
| 632 | E_{1b}E₂Eᵢᵢ | AlkLEᵥEᵥᵢ | E₄* | Eₐ |
| 633 | E₁ₐE₂EᵢᵢE_{iib} | AlkLEᵥEᵥᵢ | E₄* | Eₐ |
| 634 | E_{1b}E₂EᵢᵢE_{iib} | AlkLEᵥEᵥᵢ | E₄* | Eₐ |
| 635 | E₁ₐE₂E₃Eᵢ | AlkLEᵥEᵥᵢ | E₄* | Eₐ |
| 636 | E_{1b}E₂E₃Eᵢ | AlkLEᵥEᵥᵢ | E₄* | Eₐ |
| 637 | E₁ₐE₂E₃Eᵢᵢ | AlkLEᵥEᵥᵢ | E₄* | Eₐ |
| 638 | E_{1b}E₂E₃Eᵢᵢ | AlkLEᵥEᵥᵢ | E₄* | Eₐ |
| 639 | E₁ₐE₂E₃EᵢᵢE_{iib} | AlkLEᵥEᵥᵢ | E₄* | Eₐ |
| 640 | E_{1b}E₂E₃EᵢᵢE_{iib} | AlkLEᵥEᵥᵢ | E₄* | Eₐ |
| 641 | E₁ₐE₂E₄ | AlkLEᵥEᵥᵢ | E₄* | Eₐ |
| 642 | E_{1b}E₂E₄ | AlkLEᵥEᵥᵢ | E₄* | Eₐ |
| 643 | E₁ₐE₂E₃E₄ | AlkLEᵥEᵥᵢ | E₄* | Eₐ |
| 644 | E_{1b}E₂E₃E₄ | AlkLEᵥEᵥᵢ | E₄* | Eₐ |
| 645 | E₁ₐE₂EᵢE₄ | AlkLEᵥEᵥᵢ | E₄* | Eₐ |
| 646 | E_{1b}E₂EᵢE₄ | AlkLEᵥEᵥᵢ | E₄* | Eₐ |
| 647 | E₁ₐE₂EᵢᵢE₄ | AlkLEᵥEᵥᵢ | E₄* | Eₐ |
| 648 | E_{1b}E₂EᵢᵢE₄ | AlkLEᵥEᵥᵢ | E₄* | Eₐ |
| 649 | E₁ₐE₂EᵢᵢE_{iib}E₄ | AlkLEᵥEᵥᵢ | E₄* | Eₐ |
| 650 | E_{1b}E₂EᵢᵢE_{iib}E₄ | AlkLEᵥEᵥᵢ | E₄* | Eₐ |
| 651 | E₁ₐE₂E₃EᵢE₄ | AlkLEᵥEᵥᵢ | E₄* | Eₐ |
| 652 | E_{1b}E₂E₃EᵢE₄ | AlkLEᵥEᵥᵢ | E₄* | Eₐ |
| 653 | E₁ₐE₂E₃EᵢᵢE₄ | AlkLEᵥEᵥᵢ | E₄* | Eₐ |
| 654 | E_{1b}E₂E₃EᵢᵢE₄ | AlkLEᵥEᵥᵢ | E₄* | Eₐ |
| 655 | E₁ₐE₂E₃EᵢᵢE_{iib}E₄ | AlkLEᵥEᵥᵢ | E₄* | Eₐ |
| 656 | E_{1b}E₂E₃EᵢᵢE_{iib}E₄ | AlkLEᵥEᵥᵢ | E₄* | Eₐ |
| 657 | E₁ₐE₂Eᵢᵢᵢ | AlkLEᵥEᵥᵢ | E₄* | Eₐ |
| 658 | E_{1b}E₂Eᵢᵢᵢ | AlkLEᵥEᵥᵢ | E₄* | Eₐ |
| 659 | E₁ₐE₂E₃Eᵢᵢᵢ | AlkLEᵥEᵥᵢ | E₄* | Eₐ |
| 660 | E_{1b}E₂E₃Eᵢᵢᵢ | AlkLEᵥEᵥᵢ | E₄* | Eₐ |
| 661 | E₁ₐE₂EᵢᵢᵢE₄ | AlkLEᵥEᵥᵢ | E₄* | Eₐ |
| 662 | E_{1b}E₂EᵢᵢᵢE₄ | AlkLEᵥEᵥᵢ | E₄* | Eₐ |
| 663 | E₁ₐE₂E₃EᵢᵢᵢE₄ | AlkLEᵥEᵥᵢ | E₄* | Eₐ |
| 664 | E_{1b}E₂E₃EᵢᵢᵢE₄ | AlkLEᵥEᵥᵢ | E₄* | Eₐ |
| 665 | E₁ₐE₂Eᵢᵥ | AlkLEᵥEᵥᵢ | E₄* | Eₐ |
| 666 | E_{1b}E₂Eᵢᵥ | AlkLEᵥEᵥᵢ | E₄* | Eₐ |
| 667 | E₁ₐE₂E₃Eᵢᵥ | AlkLEᵥEᵥᵢ | E₄* | Eₐ |
| 668 | E_{1b}E₂E₃Eᵢᵥ | AlkLEᵥEᵥᵢ | E₄* | Eₐ |
| 669 | E₁ₐE₂EᵢᵥE₄ | AlkLEᵥEᵥᵢ | E₄* | Eₐ |
| 670 | E_{1b}E₂EᵢᵥE₄ | AlkLEᵥEᵥᵢ | E₄* | Eₐ |
| 671 | E₁ₐE₂E₃EᵢᵥE₄ | AlkLEᵥEᵥᵢ | E₄* | Eₐ |
| 672 | E_{1b}E₂E₃EᵢᵥE₄ | AlkLEᵥEᵥᵢ | E₄* | Eₐ |
| 673 | E₁ₐE₂ | AlkLEᵥEᵥᵢ | E₅ | Eₐ |
| 674 | E_{1b}E₂ | AlkLEᵥEᵥᵢ | E₅ | Eₐ |
| 675 | E₁ₐE₂E₃ | AlkLEᵥEᵥᵢ | E₅ | Eₐ |
| 676 | E_{1b}E₂E₃ | AlkLEᵥEᵥᵢ | E₅ | Eₐ |
| 677 | E₁ₐE₂Eᵢ | AlkLEᵥEᵥᵢ | E₅ | Eₐ |
| 678 | E_{1b}E₂Eᵢ | AlkLEᵥEᵥᵢ | E₅ | Eₐ |
| 679 | E₁ₐE₂Eᵢᵢ | AlkLEᵥEᵥᵢ | E₅ | Eₐ |
| 680 | E_{1b}E₂Eᵢᵢ | AlkLEᵥEᵥᵢ | E₅ | Eₐ |
| 681 | E₁ₐE₂EᵢᵢE_{iib} | AlkLEᵥEᵥᵢ | E₅ | Eₐ |
| 682 | E_{1b}E₂EᵢᵢE_{iib} | AlkLEᵥEᵥᵢ | E₅ | Eₐ |
| 683 | E₁ₐE₂E₃Eᵢ | AlkLEᵥEᵥᵢ | E₅ | Eₐ |
| 684 | E_{1b}E₂E₃Eᵢ | AlkLEᵥEᵥᵢ | E₅ | Eₐ |
| 685 | E₁ₐE₂E₃Eᵢᵢ | AlkLEᵥEᵥᵢ | E₅ | Eₐ |
| 686 | E_{1b}E₂E₃Eᵢᵢ | AlkLEᵥEᵥᵢ | E₅ | Eₐ |
| 687 | E₁ₐE₂E₃EᵢᵢE_{iib} | AlkLEᵥEᵥᵢ | E₅ | Eₐ |
| 688 | E_{1b}E₂E₃EᵢᵢE_{iib} | AlkLEᵥEᵥᵢ | E₅ | Eₐ |
| 689 | E₁ₐE₂E₄ | AlkLEᵥEᵥᵢ | E₅ | Eₐ |
| 690 | E_{1b}E₂E₄ | AlkLEᵥEᵥᵢ | E₅ | Eₐ |
| 691 | E₁ₐE₂E₃E₄ | AlkLEᵥEᵥᵢ | E₅ | Eₐ |
| 692 | E_{1b}E₂E₃E₄ | AlkLEᵥEᵥᵢ | E₅ | Eₐ |
| 693 | E₁ₐE₂EᵢE₄ | AlkLEᵥEᵥᵢ | E₅ | Eₐ |
| 694 | E_{1b}E₂EᵢE₄ | AlkLEᵥEᵥᵢ | E₅ | Eₐ |
| 695 | E₁ₐE₂EᵢᵢE₄ | AlkLEᵥEᵥᵢ | E₅ | Eₐ |
| 696 | E_{1b}E₂EᵢᵢE₄ | AlkLEᵥEᵥᵢ | E₅ | Eₐ |
| 697 | E₁ₐE₂EᵢᵢE_{iib}E₄ | AlkLEᵥEᵥᵢ | E₅ | Eₐ |
| 698 | E_{1b}E₂EᵢᵢE_{iib}E₄ | AlkLEᵥEᵥᵢ | E₅ | Eₐ |
| 699 | E₁ₐE₂E₃EᵢE₄ | AlkLEᵥEᵥᵢ | E₅ | Eₐ |
| 700 | E_{1b}E₂E₃EᵢE₄ | AlkLEᵥEᵥᵢ | E₅ | Eₐ |
| 701 | E₁ₐE₂E₃EᵢᵢE₄ | AlkLEᵥEᵥᵢ | E₅ | Eₐ |
| 702 | E_{1b}E₂E₃EᵢᵢE₄ | AlkLEᵥEᵥᵢ | E₅ | Eₐ |
| 703 | E₁ₐE₂E₃EᵢᵢE_{iib}E₄ | AlkLEᵥEᵥᵢ | E₅ | Eₐ |
| 704 | E_{1b}E₂E₃EᵢᵢE_{iib}E₄ | AlkLEᵥEᵥᵢ | E₅ | Eₐ |
| 705 | E₁ₐE₂Eᵢᵢᵢ | AlkLEᵥEᵥᵢ | E₅ | Eₐ |
| 706 | E_{1b}E₂Eᵢᵢᵢ | AlkLEᵥEᵥᵢ | E₅ | Eₐ |
| 707 | E₁ₐE₂E₃Eᵢᵢᵢ | AlkLEᵥEᵥᵢ | E₅ | Eₐ |
| 708 | E_{1b}E₂E₃Eᵢᵢᵢ | AlkLEᵥEᵥᵢ | E₅ | Eₐ |
| 709 | E₁ₐE₂EᵢᵢᵢE₄ | AlkLEᵥEᵥᵢ | E₅ | Eₐ |
| 710 | E_{1b}E₂EᵢᵢᵢE₄ | AlkLEᵥEᵥᵢ | E₅ | Eₐ |
| 711 | E₁ₐE₂E₃EᵢᵢᵢE₄ | AlkLEᵥEᵥᵢ | E₅ | Eₐ |
| 712 | E_{1b}E₂E₃EᵢᵢᵢE₄ | AlkLEᵥEᵥᵢ | E₅ | Eₐ |
| 713 | E₁ₐE₂Eᵢᵥ | AlkLEᵥEᵥᵢ | E₅ | Eₐ |
| 714 | E_{1b}E₂Eᵢᵥ | AlkLEᵥEᵥᵢ | E₅ | Eₐ |
| 715 | E₁ₐE₂E₃Eᵢᵥ | AlkLEᵥEᵥᵢ | E₅ | Eₐ |
| 716 | E_{1b}E₂E₃Eᵢᵥ | AlkLEᵥEᵥᵢ | E₅ | Eₐ |
| 717 | E₁ₐE₂EᵢᵥE₄ | AlkLEᵥEᵥᵢ | E₅ | Eₐ |
| 718 | E_{1b}E₂EᵢᵥE₄ | AlkLEᵥEᵥᵢ | E₅ | Eₐ |
| 719 | E₁ₐE₂E₃EᵢᵥE₄ | AlkLEᵥEᵥᵢ | E₅ | Eₐ |
| 720 | E_{1b}E₂E₃EᵢᵥE₄ | AlkLEᵥEᵥᵢ | E₅ | Eₐ |
| 721 | E₁ₐE₂ | AlkLEᵥEᵥᵢ | E₄*E₅ | Eₐ |
| 722 | E_{1b}E₂ | AlkLEᵥEᵥᵢ | E₄*E₅ | Eₐ |
| 723 | E₁ₐE₂E₃ | AlkLEᵥEᵥᵢ | E₄*E₅ | Eₐ |
| 724 | E_{1b}E₂E₃ | AlkLEᵥEᵥᵢ | E₄*E₅ | Eₐ |
| 725 | E₁ₐE₂Eᵢ | AlkLEᵥEᵥᵢ | E₄*E₅ | Eₐ |
| 726 | E_{1b}E₂Eᵢ | AlkLEᵥEᵥᵢ | E₄*E₅ | Eₐ |
| 727 | E₁ₐE₂Eᵢᵢ | AlkLEᵥEᵥᵢ | E₄*E₅ | Eₐ |
| 728 | E_{1b}E₂Eᵢᵢ | AlkLEᵥEᵥᵢ | E₄*E₅ | Eₐ |
| 729 | E₁ₐE₂EᵢᵢE_{iib} | AlkLEᵥEᵥᵢ | E₄*E₅ | Eₐ |
| 730 | E_{1b}E₂EᵢᵢE_{iib} | AlkLEᵥEᵥᵢ | E₄*E₅ | Eₐ |
| 731 | E₁ₐE₂E₃Eᵢ | AlkLEᵥEᵥᵢ | E₄*E₅ | Eₐ |
| 732 | E_{1b}E₂E₃Eᵢ | AlkLEᵥEᵥᵢ | E₄*E₅ | Eₐ |
| 733 | E₁ₐE₂E₃Eᵢᵢ | AlkLEᵥEᵥᵢ | E₄*E₅ | Eₐ |
| 734 | E_{1b}E₂E₃Eᵢᵢ | AlkLEᵥEᵥᵢ | E₄*E₅ | Eₐ |
| 735 | E₁ₐE₂E₃EᵢᵢE_{iib} | AlkLEᵥEᵥᵢ | E₄*E₅ | Eₐ |
| 736 | E_{1b}E₂E₃EᵢᵢE_{iib} | AlkLEᵥEᵥᵢ | E₄*E₅ | Eₐ |
| 737 | E₁ₐE₂E₄ | AlkLEᵥEᵥᵢ | E₄*E₅ | Eₐ |
| 738 | E_{1b}E₂E₄ | AlkLEᵥEᵥᵢ | E₄*E₅ | Eₐ |
| 739 | E₁ₐE₂E₃E₄ | AlkLEᵥEᵥᵢ | E₄*E₅ | Eₐ |
| 740 | E_{1b}E₂E₃E₄ | AlkLEᵥEᵥᵢ | E₄*E₅ | Eₐ |
| 741 | E₁ₐE₂EᵢE₄ | AlkLEᵥEᵥᵢ | E₄*E₅ | Eₐ |
| 742 | E_{1b}E₂EᵢE₄ | AlkLEᵥEᵥᵢ | E₄*E₅ | Eₐ |
| 743 | E₁ₐE₂EᵢᵢE₄ | AlkLEᵥEᵥᵢ | E₄*E₅ | Eₐ |
| 744 | E_{1b}E₂EᵢᵢE₄ | AlkLEᵥEᵥᵢ | E₄*E₅ | Eₐ |
| 745 | E₁ₐE₂EᵢᵢE_{iib}E₄ | AlkLEᵥEᵥᵢ | E₄*E₅ | Eₐ |
| 746 | E_{1b}E₂EᵢᵢE_{iib}E₄ | AlkLEᵥEᵥᵢ | E₄*E₅ | Eₐ |
| 747 | E₁ₐE₂E₃EᵢE₄ | AlkLEᵥEᵥᵢ | E₄*E₅ | Eₐ |
| 748 | E_{1b}E₂E₃EᵢE₄ | AlkLEᵥEᵥᵢ | E₄*E₅ | Eₐ |
| 749 | E₁ₐE₂E₃EᵢᵢE₄ | AlkLEᵥEᵥᵢ | E₄*E₅ | Eₐ |
| 750 | E_{1b}E₂E₃EᵢᵢE₄ | AlkLEᵥEᵥᵢ | E₄*E₅ | Eₐ |
| 751 | E₁ₐE₂E₃EᵢᵢE_{iib}E₄ | AlkLEᵥEᵥᵢ | E₄*E₅ | Eₐ |
| 752 | E_{1b}E₂E₃EᵢᵢE_{iib}E₄ | AlkLEᵥEᵥᵢ | E₄*E₅ | Eₐ |
| 753 | E₁ₐE₂Eᵢᵢᵢ | AlkLEᵥEᵥᵢ | E₄*E₅ | Eₐ |
| 754 | E_{1b}E₂Eᵢᵢᵢ | AlkLEᵥEᵥᵢ | E₄*E₅ | Eₐ |
| 755 | E₁ₐE₂E₃Eᵢᵢᵢ | AlkLEᵥEᵥᵢ | E₄*E₅ | Eₐ |
| 756 | E_{1b}E₂E₃Eᵢᵢᵢ | AlkLEᵥEᵥᵢ | E₄*E₅ | Eₐ |
| 757 | E₁ₐE₂EᵢᵢᵢE₄ | AlkLEᵥEᵥᵢ | E₄*E₅ | Eₐ |
| 758 | E_{1b}E₂EᵢᵢᵢE₄ | AlkLEᵥEᵥᵢ | E₄*E₅ | Eₐ |
| 759 | E₁ₐE₂E₃EᵢᵢᵢE₄ | AlkLEᵥEᵥᵢ | E₄*E₅ | Eₐ |
| 760 | E_{1b}E₂E₃EᵢᵢᵢE₄ | AlkLEᵥEᵥᵢ | E₄*E₅ | Eₐ |
| 761 | E₁ₐE₂Eᵢᵥ | AlkLEᵥEᵥᵢ | E₄*E₅ | Eₐ |
| 762 | E_{1b}E₂Eᵢᵥ | AlkLEᵥEᵥᵢ | E₄*E₅ | Eₐ |
| 763 | E₁ₐE₂E₃Eᵢᵥ | AlkLEᵥEᵥᵢ | E₄*E₅ | Eₐ |
| 764 | E_{1b}E₂E₃Eᵢᵥ | AlkLEᵥEᵥᵢ | E₄*E₅ | Eₐ |
| 765 | E₁ₐE₂EᵢᵥE₄ | AlkLEᵥEᵥᵢ | E₄*E₅ | Eₐ |
| 766 | E_{1b}E₂EᵢᵥE₄ | AlkLEᵥEᵥᵢ | E₄*E₅ | Eₐ |
| 767 | E₁ₐE₂E₃EᵢᵥE₄ | AlkLEᵥEᵥᵢ | E₄*E₅ | Eₐ |
| 768 | E_{1b}E₂E₃EᵢᵥE₄ | AlkLEᵥEᵥᵢ | E₄*E₅ | Eₐ |
| 769 | E₁ₐE₂ | EᵥEᵥᵢ | | E_{b} |
| 770 | E_{1b}E₂ | EᵥEᵥᵢ | | E_{b} |
| 771 | E₁ₐE₂E₃ | EᵥEᵥᵢ | | E_{b} |
| 772 | E_{1b}E₂E₃ | EᵥEᵥᵢ | | E_{b} |
| 773 | E₁ₐE₂Eᵢ | EᵥEᵥᵢ | | E_{b} |
| 774 | E_{1b}E₂Eᵢ | EᵥEᵥᵢ | | E_{b} |
| 775 | E₁ₐE₂Eᵢᵢ | EᵥEᵥᵢ | | E_{b} |
| 776 | E_{1b}E₂Eᵢᵢ | EᵥEᵥᵢ | | E_{b} |
| 777 | E₁ₐE₂EᵢᵢE_{iib} | EᵥEᵥᵢ | | E_{b} |
| 778 | E_{1b}E₂EᵢᵢE_{iib} | EᵥEᵥᵢ | | E_{b} |
| 779 | E₁ₐE₂E₃Eᵢ | EᵥEᵥᵢ | | E_{b} |
| 780 | E_{1b}E₂E₃Eᵢ | EᵥEᵥᵢ | | E_{b} |
| 781 | E₁ₐE₂E₃Eᵢᵢ | EᵥEᵥᵢ | | E_{b} |
| 782 | E_{1b}E₂E₃Eᵢᵢ | EᵥEᵥᵢ | | E_{b} |
| 783 | E₁ₐE₂E₃EᵢᵢE_{iib} | EᵥEᵥᵢ | | E_{b} |
| 784 | E_{1b}E₂E₃EᵢᵢE_{iib} | EᵥEᵥᵢ | | E_{b} |
| 785 | E₁ₐE₂E₄ | EᵥEᵥᵢ | | E_{b} |
| 786 | E_{1b}E₂E₄ | EᵥEᵥᵢ | | E_{b} |
| 787 | E₁ₐE₂E₃E₄ | EᵥEᵥᵢ | | E_{b} |
| 788 | E_{1b}E₂E₃E₄ | EᵥEᵥᵢ | | E_{b} |
| 789 | E₁ₐE₂EᵢE₄ | EᵥEᵥᵢ | | E_{b} |
| 790 | E_{1b}E₂EᵢE₄ | EᵥEᵥᵢ | | E_{b} |
| 791 | E₁ₐE₂EᵢᵢE₄ | EᵥEᵥᵢ | | E_{b} |
| 792 | E_{1b}E₂EᵢᵢE₄ | EᵥEᵥᵢ | | E_{b} |
| 793 | E₁ₐE₂EᵢᵢE_{iib}E₄ | EᵥEᵥᵢ | | E_{b} |
| 794 | E_{1b}E₂EᵢᵢE_{iib}E₄ | EᵥEᵥᵢ | | E_{b} |
| 795 | E₁ₐE₂E₃EᵢE₄ | EᵥEᵥᵢ | | E_{b} |
| 796 | E_{1b}E₂E₃EᵢE₄ | EᵥEᵥᵢ | | E_{b} |
| 797 | E₁ₐE₂E₃EᵢᵢE₄ | EᵥEᵥᵢ | | E_{b} |
| 798 | E_{1b}E₂E₃EᵢᵢE₄ | EᵥEᵥᵢ | | E_{b} |
| 799 | E₁ₐE₂E₃EᵢᵢE_{iib}E₄ | EᵥEᵥᵢ | | E_{b} |
| 800 | E_{1b}E₂E₃EᵢᵢE_{iib}E₄ | EᵥEᵥᵢ | | E_{b} |
| 801 | E₁ₐE₂Eᵢᵢᵢ | EᵥEᵥᵢ | | E_{b} |
| 802 | E_{1b}E₂Eᵢᵢᵢ | EᵥEᵥᵢ | | E_{b} |
| 803 | E₁ₐE₂E₃Eᵢᵢᵢ | EᵥEᵥᵢ | | E_{b} |
| 804 | E_{1b}E₂E₃Eᵢᵢᵢ | EᵥEᵥᵢ | | E_{b} |
| 805 | E₁ₐE₂EᵢᵢᵢE₄ | EᵥEᵥᵢ | | E_{b} |
| 806 | E_{1b}E₂EᵢᵢᵢE₄ | EᵥEᵥᵢ | | E_{b} |
| 807 | E₁ₐE₂E₃EᵢᵢᵢE₄ | EᵥEᵥᵢ | | E_{b} |
| 808 | E_{1b}E₂E₃EᵢᵢᵢE₄ | EᵥEᵥᵢ | | E_{b} |
| 809 | E₁ₐE₂Eᵢᵥ | EᵥEᵥᵢ | | E_{b} |
| 810 | E_{1b}E₂Eᵢᵥ | EᵥEᵥᵢ | | E_{b} |
| 811 | E₁ₐE₂E₃Eᵢᵥ | EᵥEᵥᵢ | | E_{b} |
| 812 | E_{1b}E₂E₃Eᵢᵥ | EᵥEᵥᵢ | | E_{b} |
| 813 | E₁ₐE₂EᵢᵥE₄ | EᵥEᵥᵢ | | E_{b} |
| 814 | E_{1b}E₂EᵢᵥE₄ | EᵥEᵥᵢ | | E_{b} |
| 815 | E₁ₐE₂E₃EᵢᵥE₄ | EᵥEᵥᵢ | | E_{b} |
| 816 | E_{1b}E₂E₃EᵢᵥE₄ | EᵥEᵥᵢ | | E_{b} |
| 817 | E₁ₐE₂ | EᵥEᵥᵢ | E₄* | E_{b} |
| 818 | E_{1b}E₂ | EᵥEᵥᵢ | E₄* | E_{b} |
| 819 | E₁ₐE₂E₃ | EᵥEᵥᵢ | E₄* | E_{b} |
| 820 | E_{1b}E₂E₃ | EᵥEᵥᵢ | E₄* | E_{b} |
| 821 | E₁ₐE₂Eᵢ | EᵥEᵥᵢ | E₄* | E_{b} |
| 822 | E_{1b}E₂Eᵢ | EᵥEᵥᵢ | E₄* | E_{b} |
| 823 | E₁ₐE₂Eᵢᵢ | EᵥEᵥᵢ | E₄* | E_{b} |
| 824 | E_{1b}E₂Eᵢᵢ | EᵥEᵥᵢ | E₄* | E_{b} |
| 825 | E₁ₐE₂EᵢᵢE_{iib} | EᵥEᵥᵢ | E₄* | E_{b} |
| 826 | E_{1b}E₂EᵢᵢE_{iib} | EᵥEᵥᵢ | E₄* | E_{b} |
| 827 | E₁ₐE₂E₃Eᵢ | EᵥEᵥᵢ | E₄* | E_{b} |
| 828 | E_{1b}E₂E₃Eᵢ | EᵥEᵥᵢ | E₄* | E_{b} |
| 829 | E₁ₐE₂E₃Eᵢᵢ | EᵥEᵥᵢ | E₄* | E_{b} |
| 830 | E_{1b}E₂E₃Eᵢᵢ | EᵥEᵥᵢ | E₄* | E_{b} |
| 831 | E₁ₐE₂E₃EᵢᵢE_{iib} | EᵥEᵥᵢ | E₄* | E_{b} |
| 832 | E_{1b}E₂E₃EᵢᵢE_{iib} | EᵥEᵥᵢ | E₄* | E_{b} |
| 833 | E₁ₐE₂E₄ | EᵥEᵥᵢ | E₄* | E_{b} |
| 834 | E_{1b}E₂E₄ | EᵥEᵥᵢ | E₄* | E_{b} |
| 835 | E₁ₐE₂E₃E₄ | EᵥEᵥᵢ | E₄* | E_{b} |
| 836 | E_{1b}E₂E₃E₄ | EᵥEᵥᵢ | E₄* | E_{b} |
| 837 | E₁ₐE₂EᵢE₄ | EᵥEᵥᵢ | E₄* | E_{b} |
| 838 | E_{1b}E₂EᵢE₄ | EᵥEᵥᵢ | E₄* | E_{b} |
| 839 | E₁ₐE₂EᵢᵢE₄ | EᵥEᵥᵢ | E₄* | E_{b} |
| 840 | E_{1b}E₂EᵢᵢE₄ | EᵥEᵥᵢ | E₄* | E_{b} |
| 841 | E₁ₐE₂EᵢᵢE_{iib}E₄ | EᵥEᵥᵢ | E₄* | E_{b} |
| 842 | E_{1b}E₂EᵢᵢE_{iib}E₄ | EᵥEᵥᵢ | E₄* | E_{b} |
| 843 | E₁ₐE₂E₃EᵢE₄ | EᵥEᵥᵢ | E₄* | E_{b} |
| 844 | E_{1b}E₂E₃EᵢE₄ | EᵥEᵥᵢ | E₄* | E_{b} |
| 845 | E₁ₐE₂E₃EᵢᵢE₄ | EᵥEᵥᵢ | E₄* | E_{b} |
| 846 | E_{1b}E₂E₃EᵢᵢE₄ | EᵥEᵥᵢ | E₄* | E_{b} |
| 847 | E₁ₐE₂E₃EᵢᵢE_{iib}E₄ | EᵥEᵥᵢ | E₄* | E_{b} |
| 848 | E_{1b}E₂E₃EᵢᵢE_{iib}E₄ | EᵥEᵥᵢ | E₄* | E_{b} |
| 849 | E₁ₐE₂Eᵢᵢᵢ | EᵥEᵥᵢ | E₄* | E_{b} |
| 850 | E_{1b}E₂Eᵢᵢᵢ | EᵥEᵥᵢ | E₄* | E_{b} |
| 851 | E₁ₐE₂E₃Eᵢᵢᵢ | EᵥEᵥᵢ | E₄* | E_{b} |
| 852 | E_{1b}E₂E₃Eᵢᵢᵢ | EᵥEᵥᵢ | E₄* | E_{b} |
| 853 | E₁ₐE₂EᵢᵢᵢE₄ | EᵥEᵥᵢ | E₄* | E_{b} |
| 854 | E_{1b}E₂EᵢᵢᵢE₄ | EᵥEᵥᵢ | E₄* | E_{b} |
| 855 | E₁ₐE₂E₃EᵢᵢᵢE₄ | EᵥEᵥᵢ | E₄* | E_{b} |
| 856 | E_{1b}E₂E₃EᵢᵢᵢE₄ | EᵥEᵥᵢ | E₄* | E_{b} |
| 857 | E₁ₐE₂Eᵢᵥ | EᵥEᵥᵢ | E₄* | E_{b} |
| 858 | E_{1b}E₂Eᵢᵥ | EᵥEᵥᵢ | E₄* | E_{b} |
| 859 | E₁ₐE₂E₃Eᵢᵥ | EᵥEᵥᵢ | E₄* | E_{b} |
| 860 | E_{1b}E₂E₃Eᵢᵥ | EᵥEᵥᵢ | E₄* | E_{b} |
| 861 | E₁ₐE₂EᵢᵥE₄ | EᵥEᵥᵢ | E₄* | E_{b} |
| 862 | E_{1b}E₂EᵢᵥE₄ | EᵥEᵥᵢ | E₄* | E_{b} |
| 863 | E₁ₐE₂E₃EᵢᵥE₄ | EᵥEᵥᵢ | E₄* | E_{b} |
| 864 | E_{1b}E₂E₃EᵢᵥE₄ | EᵥEᵥᵢ | E₄* | E_{b} |
| 865 | E₁ₐE₂ | EᵥEᵥᵢ | E₅ | E_{b} |
| 866 | E_{1b}E₂ | EᵥEᵥᵢ | E₅ | E_{b} |
| 867 | E₁ₐE₂E₃ | EᵥEᵥᵢ | E₅ | E_{b} |
| 868 | E_{1b}E₂E₃ | EᵥEᵥᵢ | E₅ | E_{b} |
| 869 | E₁ₐE₂Eᵢ | EᵥEᵥᵢ | E₅ | E_{b} |
| 870 | E_{1b}E₂Eᵢ | EᵥEᵥᵢ | E₅ | E_{b} |
| 871 | E₁ₐE₂Eᵢᵢ | EᵥEᵥᵢ | E₅ | E_{b} |
| 872 | E_{1b}E₂Eᵢᵢ | EᵥEᵥᵢ | E₅ | E_{b} |
| 873 | E₁ₐE₂EᵢᵢE_{iib} | EᵥEᵥᵢ | E₅ | E_{b} |
| 874 | E_{1b}E₂EᵢᵢE_{iib} | EᵥEᵥᵢ | E₅ | E_{b} |
| 875 | E₁ₐE₂E₃Eᵢ | EᵥEᵥᵢ | E₅ | E_{b} |
| 876 | E_{1b}E₂E₃Eᵢ | EᵥEᵥᵢ | E₅ | E_{b} |
| 877 | E₁ₐE₂E₃Eᵢᵢ | EᵥEᵥᵢ | E₅ | E_{b} |
| 878 | E_{1b}E₂E₃Eᵢᵢ | EᵥEᵥᵢ | E₅ | E_{b} |
| 879 | E₁ₐE₂E₃EᵢᵢE_{iib} | EᵥEᵥᵢ | E₅ | E_{b} |
| 880 | E_{1b}E₂E₃EᵢᵢE_{iib} | EᵥEᵥᵢ | E₅ | E_{b} |
| 881 | E₁ₐE₂E₄ | EᵥEᵥᵢ | E₅ | E_{b} |
| 882 | E_{1b}E₂E₄ | EᵥEᵥᵢ | E₅ | E_{b} |
| 883 | E₁ₐE₂E₃E₄ | EᵥEᵥᵢ | E₅ | E_{b} |
| 884 | E_{1b}E₂E₃E₄ | EᵥEᵥᵢ | E₅ | E_{b} |
| 885 | E₁ₐE₂EᵢE₄ | EᵥEᵥᵢ | E₅ | E_{b} |
| 886 | E_{1b}E₂EᵢE₄ | EᵥEᵥᵢ | E₅ | E_{b} |
| 887 | E₁ₐE₂EᵢᵢE₄ | EᵥEᵥᵢ | E₅ | E_{b} |
| 888 | E_{1b}E₂EᵢᵢE₄ | EᵥEᵥᵢ | E₅ | E_{b} |
| 889 | E₁ₐE₂EᵢᵢE_{iib}E₄ | EᵥEᵥᵢ | E₅ | E_{b} |
| 890 | E_{1b}E₂EᵢᵢE_{iib}E₄ | EᵥEᵥᵢ | E₅ | E_{b} |
| 891 | E₁ₐE₂E₃EᵢE₄ | EᵥEᵥᵢ | E₅ | E_{b} |
| 892 | E_{1b}E₂E₃EᵢE₄ | EᵥEᵥᵢ | E₅ | E_{b} |
| 893 | E₁ₐE₂E₃EᵢᵢE₄ | EᵥEᵥᵢ | E₅ | E_{b} |
| 894 | E_{1b}E₂E₃EᵢᵢE₄ | EᵥEᵥᵢ | E₅ | E_{b} |
| 895 | E₁ₐE₂E₃EᵢᵢE_{iib}E₄ | EᵥEᵥᵢ | E₅ | E_{b} |
| 896 | E_{1b}E₂E₃EᵢᵢE_{iib}E₄ | EᵥEᵥᵢ | E₅ | E_{b} |
| 897 | E₁ₐE₂Eᵢᵢᵢ | EᵥEᵥᵢ | E₅ | E_{b} |
| 898 | E_{1b}E₂Eᵢᵢᵢ | EᵥEᵥᵢ | E₅ | E_{b} |
| 899 | E₁ₐE₂E₃Eᵢᵢᵢ | EᵥEᵥᵢ | E₅ | E_{b} |
| 900 | E_{1b}E₂E₃Eᵢᵢᵢ | EᵥEᵥᵢ | E₅ | E_{b} |
| 901 | E₁ₐE₂EᵢᵢᵢE₄ | EᵥEᵥᵢ | E₅ | E_{b} |
| 902 | E_{1b}E₂EᵢᵢᵢE₄ | EᵥEᵥᵢ | E₅ | E_{b} |
| 903 | E₁ₐE₂E₃EᵢᵢᵢE₄ | EᵥEᵥᵢ | E₅ | E_{b} |
| 904 | E_{1b}E₂E₃EᵢᵢᵢE₄ | EᵥEᵥᵢ | E₅ | E_{b} |
| 905 | E₁ₐE₂Eᵢᵥ | EᵥEᵥᵢ | E₅ | E_{b} |
| 906 | E_{1b}E₂Eᵢᵥ | EᵥEᵥᵢ | E₅ | E_{b} |
| 907 | E₁ₐE₂E₃Eᵢᵥ | EᵥEᵥᵢ | E₅ | E_{b} |
| 908 | E_{1b}E₂E₃Eᵢᵥ | EᵥEᵥᵢ | E₅ | E_{b} |
| 909 | E₁ₐE₂EᵢᵥE₄ | EᵥEᵥᵢ | E₅ | E_{b} |
| 910 | E_{1b}E₂EᵢᵥE₄ | EᵥEᵥᵢ | E₅ | E_{b} |
| 911 | E₁ₐE₂E₃EᵢᵥE₄ | EᵥEᵥᵢ | E₅ | E_{b} |
| 912 | E_{1b}E₂E₃EᵢᵥE₄ | EᵥEᵥᵢ | E₅ | E_{b} |
| 913 | E₁ₐE₂ | EᵥEᵥᵢ | E₄*E₅ | E_{b} |
| 914 | E_{1b}E₂ | EᵥEᵥᵢ | E₄*E₅ | E_{b} |
| 915 | E₁ₐE₂E₃ | EᵥEᵥᵢ | E₄*E₅ | E_{b} |
| 916 | E_{1b}E₂E₃ | EᵥEᵥᵢ | E₄*E₅ | E_{b} |
| 917 | E₁ₐE₂Eᵢ | EᵥEᵥᵢ | E₄*E₅ | E_{b} |
| 918 | E_{1b}E₂Eᵢ | EᵥEᵥᵢ | E₄*E₅ | E_{b} |
| 919 | E₁ₐE₂Eᵢᵢ | EᵥEᵥᵢ | E₄*E₅ | E_{b} |
| 920 | E_{1b}E₂Eᵢᵢ | EᵥEᵥᵢ | E₄*E₅ | E_{b} |
| 921 | E₁ₐE₂EᵢᵢE_{iib} | EᵥEᵥᵢ | E₄*E₅ | E_{b} |
| 922 | E_{1b}E₂EᵢᵢE_{iib} | EᵥEᵥᵢ | E₄*E₅ | E_{b} |
| 923 | E₁ₐE₂E₃Eᵢ | EᵥEᵥᵢ | E₄*E₅ | E_{b} |
| 924 | E_{1b}E₂E₃Eᵢ | EᵥEᵥᵢ | E₄*E₅ | E_{b} |
| 925 | E₁ₐE₂E₃Eᵢᵢ | EᵥEᵥᵢ | E₄*E₅ | E_{b} |
| 926 | E_{1b}E₂E₃Eᵢᵢ | EᵥEᵥᵢ | E₄*E₅ | E_{b} |
| 927 | E₁ₐE₂E₃EᵢᵢE_{iib} | EᵥEᵥᵢ | E₄*E₅ | E_{b} |
| 928 | E_{1b}E₂E₃EᵢᵢE_{iib} | EᵥEᵥᵢ | E₄*E₅ | E_{b} |
| 929 | E₁ₐE₂E₄ | EᵥEᵥᵢ | E₄*E₅ | E_{b} |
| 930 | E_{1b}E₂E₄ | EᵥEᵥᵢ | E₄*E₅ | E_{b} |
| 931 | E₁ₐE₂E₃E₄ | EᵥEᵥᵢ | E₄*E₅ | E_{b} |
| 932 | E_{1b}E₂E₃E₄ | EᵥEᵥᵢ | E₄*E₅ | E_{b} |
| 933 | E₁ₐE₂EᵢE₄ | EᵥEᵥᵢ | E₄*E₅ | E_{b} |
| 934 | E_{1b}E₂EᵢE₄ | EᵥEᵥᵢ | E₄*E₅ | E_{b} |
| 935 | E₁ₐE₂EᵢᵢE₄ | EᵥEᵥᵢ | E₄*E₅ | E_{b} |
| 936 | E_{1b}E₂EᵢᵢE₄ | EᵥEᵥᵢ | E₄*E₅ | E_{b} |
| 937 | E₁ₐE₂EᵢᵢE_{iib}E₄ | EᵥEᵥᵢ | E₄*E₅ | E_{b} |
| 938 | E_{1b}E₂EᵢᵢE_{iib}E₄ | EᵥEᵥᵢ | E₄*E₅ | E_{b} |
| 939 | E₁ₐE₂E₃EᵢE₄ | EᵥEᵥᵢ | E₄*E₅ | E_{b} |
| 940 | E_{1b}E₂E₃EᵢE₄ | EᵥEᵥᵢ | E₄*E₅ | E_{b} |
| 941 | E₁ₐE₂E₃EᵢᵢE₄ | EᵥEᵥᵢ | E₄*E₅ | E_{b} |
| 942 | E_{1b}E₂E₃EᵢᵢE₄ | EᵥEᵥᵢ | E₄*E₅ | E_{b} |
| 943 | E₁ₐE₂E₃EᵢᵢE_{iib}E₄ | EᵥEᵥᵢ | E₄*E₅ | E_{b} |
| 944 | E_{1b}E₂E₃EᵢᵢE_{iib}E₄ | EᵥEᵥᵢ | E₄*E₅ | E_{b} |
| 945 | E₁ₐE₂Eᵢᵢᵢ | EᵥEᵥᵢ | E₄*E₅ | E_{b} |
| 946 | E_{1b}E₂Eᵢᵢᵢ | EᵥEᵥᵢ | E₄*E₅ | E_{b} |
| 947 | E₁ₐE₂E₃Eᵢᵢᵢ | EᵥEᵥᵢ | E₄*E₅ | E_{b} |
| 948 | E_{1b}E₂E₃Eᵢᵢᵢ | EᵥEᵥᵢ | E₄*E₅ | E_{b} |
| 949 | E₁ₐE₂EᵢᵢᵢE₄ | EᵥEᵥᵢ | E₄*E₅ | E_{b} |
| 950 | E_{1b}E₂EᵢᵢᵢE₄ | EᵥEᵥᵢ | E₄*E₅ | E_{b} |
| 951 | E₁ₐE₂E₃EᵢᵢᵢE₄ | EᵥEᵥᵢ | E₄*E₅ | E_{b} |
| 952 | E_{1b}E₂E₃EᵢᵢᵢE₄ | EᵥEᵥᵢ | E₄*E₅ | E_{b} |
| 953 | E₁ₐE₂Eᵢᵥ | EᵥEᵥᵢ | E₄*E₅ | E_{b} |
| 954 | E_{1b}E₂Eᵢᵥ | EᵥEᵥᵢ | E₄*E₅ | E_{b} |
| 955 | E₁ₐE₂E₃Eᵢᵥ | EᵥEᵥᵢ | E₄*E₅ | E_{b} |
| 956 | E_{1b}E₂E₃Eᵢᵥ | EᵥEᵥᵢ | E₄*E₅ | E_{b} |
| 957 | E₁ₐE₂EᵢᵥE₄ | EᵥEᵥᵢ | E₄*E₅ | E_{b} |
| 958 | E_{1b}E₂EᵢᵥE₄ | EᵥEᵥᵢ | E₄*E₅ | E_{b} |
| 959 | E₁ₐE₂E₃EᵢᵥE₄ | EᵥEᵥᵢ | E₄*E₅ | E_{b} |
| 960 | E_{1b}E₂E₃EᵢᵥE₄ | EᵥEᵥᵢ | E₄*E₅ | E_{b} |
| 961 | E₁ₐE₂ | AlkLEᵥEᵥᵢ | | E_{b} |
| 962 | E_{1b}E₂ | AlkLEᵥEᵥᵢ | | E_{b} |
| 963 | E₁ₐE₂E₃ | AlkLEᵥEᵥᵢ | | E_{b} |
| 964 | E_{1b}E₂E₃ | AlkLEᵥEᵥᵢ | | E_{b} |
| 965 | E₁ₐE₂Eᵢ | AlkLEᵥEᵥᵢ | | E_{b} |
| 966 | E_{1b}E₂Eᵢ | AlkLEᵥEᵥᵢ | | E_{b} |
| 967 | E₁ₐE₂Eᵢᵢ | AlkLEᵥEᵥᵢ | | E_{b} |
| 968 | E_{1b}E₂Eᵢᵢ | AlkLEᵥEᵥᵢ | | E_{b} |
| 969 | E₁ₐE₂EᵢᵢE_{iib} | AlkLEᵥEᵥᵢ | | E_{b} |
| 970 | E_{1b}E₂EᵢᵢE_{iib} | AlkLEᵥEᵥᵢ | | E_{b} |
| 971 | E₁ₐE₂E₃Eᵢ | AlkLEᵥEᵥᵢ | | E_{b} |
| 972 | E_{1b}E₂E₃Eᵢ | AlkLEᵥEᵥᵢ | | E_{b} |
| 973 | E₁ₐE₂E₃Eᵢᵢ | AlkLEᵥEᵥᵢ | | E_{b} |
| 974 | E_{1b}E₂E₃Eᵢᵢ | AlkLEᵥEᵥᵢ | | E_{b} |
| 975 | E₁ₐE₂E₃EᵢᵢE_{iib} | AlkLEᵥEᵥᵢ | | E_{b} |
| 976 | E_{1b}E₂E₃EᵢᵢE_{iib} | AlkLEᵥEᵥᵢ | | E_{b} |
| 977 | E₁ₐE₂E₄ | AlkLEᵥEᵥᵢ | | E_{b} |
| 978 | E_{1b}E₂E₄ | AlkLEᵥEᵥᵢ | | E_{b} |
| 979 | E₁ₐE₂E₃E₄ | AlkLEᵥEᵥᵢ | | E_{b} |
| 980 | E_{1b}E₂E₃E₄ | AlkLEᵥEᵥᵢ | | E_{b} |
| 981 | E₁ₐE₂EᵢE₄ | AlkLEᵥEᵥᵢ | | E_{b} |
| 982 | E_{1b}E₂EᵢE₄ | AlkLEᵥEᵥᵢ | | E_{b} |
| 983 | E₁ₐE₂EᵢᵢE₄ | AlkLEᵥEᵥᵢ | | E_{b} |
| 984 | E_{1b}E₂EᵢᵢE₄ | AlkLEᵥEᵥᵢ | | E_{b} |
| 985 | E₁ₐE₂EᵢᵢE_{iib}E₄ | AlkLEᵥEᵥᵢ | | E_{b} |
| 986 | E_{1b}E₂EᵢᵢE_{iib}E₄ | AlkLEᵥEᵥᵢ | | E_{b} |
| 987 | E₁ₐE₂E₃EᵢE₄ | AlkLEᵥEᵥᵢ | | E_{b} |
| 988 | E_{1b}E₂E₃EᵢE₄ | AlkLEᵥEᵥᵢ | | E_{b} |
| 989 | E₁ₐE₂E₃EᵢᵢE₄ | AlkLEᵥEᵥᵢ | | E_{b} |
| 990 | E_{1b}E₂E₃EᵢᵢE₄ | AlkLEᵥEᵥᵢ | | E_{b} |
| 991 | E₁ₐE₂E₃EᵢᵢE_{iib}E₄ | AlkLEᵥEᵥᵢ | | E_{b} |
| 992 | E_{1b}E₂E₃EᵢᵢE_{iib}E₄ | AlkLEᵥEᵥᵢ | | E_{b} |
| 993 | E₁ₐE₂Eᵢᵢᵢ | AlkLEᵥEᵥᵢ | | E_{b} |
| 994 | E_{1b}E₂Eᵢᵢᵢ | AlkLEᵥEᵥᵢ | | E_{b} |
| 995 | E₁ₐE₂E₃Eᵢᵢᵢ | AlkLEᵥEᵥᵢ | | E_{b} |
| 996 | E_{1b}E₂E₃Eᵢᵢᵢ | AlkLEᵥEᵥᵢ | | E_{b} |
| 997 | E₁ₐE₂EᵢᵢᵢE₄ | AlkLEᵥEᵥᵢ | | E_{b} |
| 998 | E_{1b}E₂EᵢᵢᵢE₄ | AlkLEᵥEᵥᵢ | | E_{b} |
| 999 | E₁ₐE₂E₃EᵢᵢᵢE₄ | AlkLEᵥEᵥᵢ | | E_{b} |
| 1000 | E_{1b}E₂E₃EᵢᵢᵢE₄ | AlkLEᵥEᵥᵢ | | E_{b} |
| 1001 | E₁ₐE₂Eᵢᵥ | AlkLEᵥEᵥᵢ | | E_{b} |
| 1002 | E_{1b}E₂Eᵢᵥ | AlkLEᵥEᵥᵢ | | E_{b} |
| 1003 | E₁ₐE₂E₃Eᵢᵥ | AlkLEᵥEᵥᵢ | | E_{b} |
| 1004 | E_{1b}E₂E₃Eᵢᵥ | AlkLEᵥEᵥᵢ | | E_{b} |
| 1005 | E₁ₐE₂EᵢᵥE₄ | AlkLEᵥEᵥᵢ | | E_{b} |
| 1006 | E_{1b}E₂EᵢᵥE₄ | AlkLEᵥEᵥᵢ | | E_{b} |
| 1007 | E₁ₐE₂E₃EᵢᵥE₄ | AlkLEᵥEᵥᵢ | | E_{b} |
| 1008 | E_{1b}E₂E₃EᵢᵥE₄ | AlkLEᵥEᵥᵢ | | E_{b} |
| 1009 | E₁ₐE₂ | AlkLEᵥEᵥᵢ | E₄* | E_{b} |
| 1010 | E_{1b}E₂ | AlkLEᵥEᵥᵢ | E₄* | E_{b} |
| 1011 | E₁ₐE₂E₃ | AlkLEᵥEᵥᵢ | E₄* | E_{b} |
| 1012 | E_{1b}E₂E₃ | AlkLEᵥEᵥᵢ | E₄* | E_{b} |
| 1013 | E₁ₐE₂Eᵢ | AlkLEᵥEᵥᵢ | E₄* | E_{b} |
| 1014 | E_{1b}E₂Eᵢ | AlkLEᵥEᵥᵢ | E₄* | E_{b} |
| 1015 | E₁ₐE₂Eᵢᵢ | AlkLEᵥEᵥᵢ | E₄* | E_{b} |
| 1016 | E_{1b}E₂Eᵢᵢ | AlkLEᵥEᵥᵢ | E₄* | E_{b} |
| 1017 | E₁ₐE₂EᵢᵢE_{iib} | AlkLEᵥEᵥᵢ | E₄* | E_{b} |
| 1018 | E_{1b}E₂EᵢᵢE_{iib} | AlkLEᵥEᵥᵢ | E₄* | E_{b} |
| 1019 | E₁ₐE₂E₃Eᵢ | AlkLEᵥEᵥᵢ | E₄* | E_{b} |
| 1020 | E_{1b}E₂E₃Eᵢ | AlkLEᵥEᵥᵢ | E₄* | E_{b} |
| 1021 | E₁ₐE₂E₃Eᵢᵢ | AlkLEᵥEᵥᵢ | E₄* | E_{b} |
| 1022 | E_{1b}E₂E₃Eᵢᵢ | AlkLEᵥEᵥᵢ | E₄* | E_{b} |
| 1023 | E₁ₐE₂E₃EᵢᵢE_{iib} | AlkLEᵥEᵥᵢ | E₄* | E_{b} |
| 1024 | E_{1b}E₂E₃EᵢᵢE_{iib} | AlkLEᵥEᵥᵢ | E₄* | E_{b} |
| 1025 | E₁ₐE₂E₄ | AlkLEᵥEᵥᵢ | E₄* | E_{b} |
| 1026 | E_{1b}E₂E₄ | AlkLEᵥEᵥᵢ | E₄* | E_{b} |
| 1027 | E₁ₐE₂E₃E₄ | AlkLEᵥEᵥᵢ | E₄* | E_{b} |
| 1028 | E_{1b}E₂E₃E₄ | AlkLEᵥEᵥᵢ | E₄* | E_{b} |
| 1029 | E₁ₐE₂EᵢE₄ | AlkLEᵥEᵥᵢ | E₄* | E_{b} |
| 1030 | E_{1b}E₂EᵢE₄ | AlkLEᵥEᵥᵢ | E₄* | E_{b} |
| 1031 | E₁ₐE₂EᵢᵢE₄ | AlkLEᵥEᵥᵢ | E₄* | E_{b} |
| 1032 | E_{1b}E₂EᵢᵢE₄ | AlkLEᵥEᵥᵢ | E₄* | E_{b} |
| 1033 | E₁ₐE₂EᵢᵢE_{iib}E₄ | AlkLEᵥEᵥᵢ | E₄* | E_{b} |
| 1034 | E_{1b}E₂EᵢᵢE_{iib}E₄ | AlkLEᵥEᵥᵢ | E₄* | E_{b} |
| 1035 | E₁ₐE₂E₃EᵢE₄ | AlkLEᵥEᵥᵢ | E₄* | E_{b} |
| 1036 | E_{1b}E₂E₃EᵢE₄ | AlkLEᵥEᵥᵢ | E₄* | E_{b} |
| 1037 | E₁ₐE₂E₃EᵢᵢE₄ | AlkLEᵥEᵥᵢ | E₄* | E_{b} |
| 1038 | E_{1b}E₂E₃EᵢᵢE₄ | AlkLEᵥEᵥᵢ | E₄* | E_{b} |
| 1039 | E₁ₐE₂E₃EᵢᵢE_{iib}E₄ | AlkLEᵥEᵥᵢ | E₄* | E_{b} |
| 1040 | E_{1b}E₂E₃EᵢᵢE_{iib}E₄ | AlkLEᵥEᵥᵢ | E₄* | E_{b} |
| 1041 | E₁ₐE₂Eᵢᵢᵢ | AlkLEᵥEᵥᵢ | E₄* | E_{b} |
| 1042 | E_{1b}E₂Eᵢᵢᵢ | AlkLEᵥEᵥᵢ | E₄* | E_{b} |
| 1043 | E₁ₐE₂E₃Eᵢᵢᵢ | AlkLEᵥEᵥᵢ | E₄* | E_{b} |
| 1044 | E_{1b}E₂E₃Eᵢᵢᵢ | AlkLEᵥEᵥᵢ | E₄* | E_{b} |
| 1045 | E₁ₐE₂EᵢᵢᵢE₄ | AlkLEᵥEᵥᵢ | E₄* | E_{b} |
| 1046 | E_{1b}E₂EᵢᵢᵢE₄ | AlkLEᵥEᵥᵢ | E₄* | E_{b} |
| 1047 | E₁ₐE₂E₃EᵢᵢᵢE₄ | AlkLEᵥEᵥᵢ | E₄* | E_{b} |
| 1048 | E_{1b}E₂E₃EᵢᵢᵢE₄ | AlkLEᵥEᵥᵢ | E₄* | E_{b} |
| 1049 | E₁ₐE₂Eᵢᵥ | AlkLEᵥEᵥᵢ | E₄* | E_{b} |
| 1050 | E_{1b}E₂Eᵢᵥ | AlkLEᵥEᵥᵢ | E₄* | E_{b} |
| 1051 | E₁ₐE₂E₃Eᵢᵥ | AlkLEᵥEᵥᵢ | E₄* | E_{b} |
| 1052 | E_{1b}E₂E₃Eᵢᵥ | AlkLEᵥEᵥᵢ | E₄* | E_{b} |
| 1053 | E₁ₐE₂EᵢᵥE₄ | AlkLEᵥEᵥᵢ | E₄* | E_{b} |
| 1054 | E_{1b}E₂EᵢᵥE₄ | AlkLEᵥEᵥᵢ | E₄* | E_{b} |
| 1055 | E₁ₐE₂E₃EᵢᵥE₄ | AlkLEᵥEᵥᵢ | E₄* | E_{b} |
| 1056 | E_{1b}E₂E₃EᵢᵥE₄ | AlkLEᵥEᵥᵢ | E₄* | E_{b} |
| 1057 | E₁ₐE₂ | AlkLEᵥEᵥᵢ | E₅ | E_{b} |
| 1058 | E_{1b}E₂ | AlkLEᵥEᵥᵢ | E₅ | E_{b} |
| 1059 | E₁ₐE₂E₃ | AlkLEᵥEᵥᵢ | E₅ | E_{b} |
| 1060 | E_{1b}E₂E₃ | AlkLEᵥEᵥᵢ | E₅ | E_{b} |
| 1061 | E₁ₐE₂Eᵢ | AlkLEᵥEᵥᵢ | E₅ | E_{b} |
| 1062 | E_{1b}E₂Eᵢ | AlkLEᵥEᵥᵢ | E₅ | E_{b} |
| 1063 | E₁ₐE₂Eᵢᵢ | AlkLEᵥEᵥᵢ | E₅ | E_{b} |
| 1064 | E_{1b}E₂Eᵢᵢ | AlkLEᵥEᵥᵢ | E₅ | E_{b} |
| 1065 | E₁ₐE₂EᵢᵢE_{iib} | AlkLEᵥEᵥᵢ | E₅ | E_{b} |
| 1066 | E_{1b}E₂EᵢᵢE_{iib} | AlkLEᵥEᵥᵢ | E₅ | E_{b} |
| 1067 | E₁ₐE₂E₃Eᵢ | AlkLEᵥEᵥᵢ | E₅ | E_{b} |
| 1068 | E_{1b}E₂E₃Eᵢ | AlkLEᵥEᵥᵢ | E₅ | E_{b} |
| 1069 | E₁ₐE₂E₃Eᵢᵢ | AlkLEᵥEᵥᵢ | E₅ | E_{b} |
| 1070 | E_{1b}E₂E₃Eᵢᵢ | AlkLEᵥEᵥᵢ | E₅ | E_{b} |
| 1071 | E₁ₐE₂E₃EᵢᵢE_{iib} | AlkLEᵥEᵥᵢ | E₅ | E_{b} |
| 1072 | E_{1b}E₂E₃EᵢᵢE_{iib} | AlkLEᵥEᵥᵢ | E₅ | E_{b} |
| 1073 | E₁ₐE₂E₄ | AlkLEᵥEᵥᵢ | E₅ | E_{b} |
| 1074 | E_{1b}E₂E₄ | AlkLEᵥEᵥᵢ | E₅ | E_{b} |
| 1075 | E₁ₐE₂E₃E₄ | AlkLEᵥEᵥᵢ | E₅ | E_{b} |
| 1076 | E_{1b}E₂E₃E₄ | AlkLEᵥEᵥᵢ | E₅ | E_{b} |
| 1077 | E₁ₐE₂EᵢE₄ | AlkLEᵥEᵥᵢ | E₅ | E_{b} |
| 1078 | E_{1b}E₂EᵢE₄ | AlkLEᵥEᵥᵢ | E₅ | E_{b} |
| 1079 | E₁ₐE₂EᵢᵢE₄ | AlkLEᵥEᵥᵢ | E₅ | E_{b} |
| 1080 | E_{1b}E₂EᵢᵢE₄ | AlkLEᵥEᵥᵢ | E₅ | E_{b} |
| 1081 | E₁ₐE₂EᵢᵢE_{iib}E₄ | AlkLEᵥEᵥᵢ | E₅ | E_{b} |
| 1082 | E_{1b}E₂EᵢᵢE_{iib}E₄ | AlkLEᵥEᵥᵢ | E₅ | E_{b} |
| 1083 | E₁ₐE₂E₃EᵢE₄ | AlkLEᵥEᵥᵢ | E₅ | E_{b} |
| 1084 | E_{1b}E₂E₃EᵢE₄ | AlkLEᵥEᵥᵢ | E₅ | E_{b} |
| 1085 | E₁ₐE₂E₃EᵢᵢE₄ | AlkLEᵥEᵥᵢ | E₅ | E_{b} |
| 1086 | E_{1b}E₂E₃EᵢᵢE₄ | AlkLEᵥEᵥᵢ | E₅ | E_{b} |
| 1087 | E₁ₐE₂E₃EᵢᵢE_{iib}E₄ | AlkLEᵥEᵥᵢ | E₅ | E_{b} |
| 1088 | E_{1b}E₂E₃EᵢᵢE_{iib}E₄ | AlkLEᵥEᵥᵢ | E₅ | E_{b} |
| 1089 | E₁ₐE₂Eᵢᵢᵢ | AlkLEᵥEᵥᵢ | E₅ | E_{b} |
| 1090 | E_{1b}E₂Eᵢᵢᵢ | AlkLEᵥEᵥᵢ | E₅ | E_{b} |
| 1091 | E₁ₐE₂E₃Eᵢᵢᵢ | AlkLEᵥEᵥᵢ | E₅ | E_{b} |
| 1092 | E_{1b}E₂E₃Eᵢᵢᵢ | AlkLEᵥEᵥᵢ | E₅ | E_{b} |
| 1093 | E₁ₐE₂EᵢᵢᵢE₄ | AlkLEᵥEᵥᵢ | E₅ | E_{b} |
| 1094 | E_{1b}E₂EᵢᵢᵢE₄ | AlkLEᵥEᵥᵢ | E₅ | E_{b} |
| 1095 | E₁ₐE₂E₃EᵢᵢᵢE₄ | AlkLEᵥEᵥᵢ | E₅ | E_{b} |
| 1096 | E_{1b}E₂E₃EᵢᵢᵢE₄ | AlkLEᵥEᵥᵢ | E₅ | E_{b} |
| 1097 | E₁ₐE₂Eᵢᵥ | AlkLEᵥEᵥᵢ | E₅ | E_{b} |
| 1098 | E_{1b}E₂Eᵢᵥ | AlkLEᵥEᵥᵢ | E₅ | E_{b} |
| 1099 | E₁ₐE₂E₃Eᵢᵥ | AlkLEᵥEᵥᵢ | E₅ | E_{b} |
| 1100 | E_{1b}E₂E₃Eᵢᵥ | AlkLEᵥEᵥᵢ | E₅ | E_{b} |
| 1101 | E₁ₐE₂EᵢᵥE₄ | AlkLEᵥEᵥᵢ | E₅ | E_{b} |
| 1102 | E_{1b}E₂EᵢᵥE₄ | AlkLEᵥEᵥᵢ | E₅ | E_{b} |
| 1103 | E₁ₐE₂E₃EᵢᵥE₄ | AlkLEᵥEᵥᵢ | E₅ | E_{b} |
| 1104 | E_{1b}E₂E₃EᵢᵥE₄ | AlkLEᵥEᵥᵢ | E₅ | E_{b} |
| 1105 | E₁ₐE₂ | AlkLEᵥEᵥᵢ | E₄*E₅ | E_{b} |
| 1106 | E_{1b}E₂ | AlkLEᵥEᵥᵢ | E₄*E₅ | E_{b} |
| 1107 | E₁ₐE₂E₃ | AlkLEᵥEᵥᵢ | E₄*E₅ | E_{b} |
| 1108 | E_{1b}E₂E₃ | AlkLEᵥEᵥᵢ | E₄*E₅ | E_{b} |
| 1109 | E₁ₐE₂Eᵢ | AlkLEᵥEᵥᵢ | E₄*E₅ | E_{b} |
| 1110 | E_{1b}E₂Eᵢ | AlkLEᵥEᵥᵢ | E₄*E₅ | E_{b} |
| 1111 | E₁ₐE₂Eᵢᵢ | AlkLEᵥEᵥᵢ | E₄*E₅ | E_{b} |
| 1112 | E_{1b}E₂Eᵢᵢ | AlkLEᵥEᵥᵢ | E₄*E₅ | E_{b} |
| 1113 | E₁ₐE₂EᵢᵢE_{iib} | AlkLEᵥEᵥᵢ | E₄*E₅ | E_{b} |
| 1114 | E_{1b}E₂EᵢᵢE_{iib} | AlkLEᵥEᵥᵢ | E₄*E₅ | E_{b} |
| 1115 | E₁ₐE₂E₃Eᵢ | AlkLEᵥEᵥᵢ | E₄*E₅ | E_{b} |
| 1116 | E_{1b}E₂E₃Eᵢ | AlkLEᵥEᵥᵢ | E₄*E₅ | E_{b} |
| 1117 | E₁ₐE₂E₃Eᵢᵢ | AlkLEᵥEᵥᵢ | E₄*E₅ | E_{b} |
| 1118 | E_{1b}E₂E₃Eᵢᵢ | AlkLEᵥEᵥᵢ | E₄*E₅ | E_{b} |
| 1119 | E₁ₐE₂E₃EᵢᵢE_{iib} | AlkLEᵥEᵥᵢ | E₄*E₅ | E_{b} |
| 1120 | E_{1b}E₂E₃EᵢᵢE_{iib} | AlkLEᵥEᵥᵢ | E₄*E₅ | E_{b} |
| 1121 | E₁ₐE₂E₄ | AlkLEᵥEᵥᵢ | E₄*E₅ | E_{b} |
| 1122 | E_{1b}E₂E₄ | AlkLEᵥEᵥᵢ | E₄*E₅ | E_{b} |
| 1123 | E₁ₐE₂E₃E₄ | AlkLEᵥEᵥᵢ | E₄*E₅ | E_{b} |
| 1124 | E_{1b}E₂E₃E₄ | AlkLEᵥEᵥᵢ | E₄*E₅ | E_{b} |
| 1125 | E₁ₐE₂EᵢE₄ | AlkLEᵥEᵥᵢ | E₄*E₅ | E_{b} |
| 1126 | E_{1b}E₂EᵢE₄ | AlkLEᵥEᵥᵢ | E₄*E₅ | E_{b} |
| 1127 | E₁ₐE₂EᵢᵢE₄ | AlkLEᵥEᵥᵢ | E₄*E₅ | E_{b} |
| 1128 | E_{1b}E₂EᵢᵢE₄ | AlkLEᵥEᵥᵢ | E₄*E₅ | E_{b} |
| 1129 | E₁ₐE₂EᵢᵢE_{iib}E₄ | AlkLEᵥEᵥᵢ | E₄*E₅ | E_{b} |
| 1130 | E_{1b}E₂EᵢᵢE_{iib}E₄ | AlkLEᵥEᵥᵢ | E₄*E₅ | E_{b} |
| 1131 | E₁ₐE₂E₃EᵢE₄ | AlkLEᵥEᵥᵢ | E₄*E₅ | E_{b} |
| 1132 | E_{1b}E₂E₃EᵢE₄ | AlkLEᵥEᵥᵢ | E₄*E₅ | E_{b} |
| 1133 | E₁ₐE₂E₃EᵢᵢE₄ | AlkLEᵥEᵥᵢ | E₄*E₅ | E_{b} |
| 1134 | E_{1b}E₂E₃EᵢᵢE₄ | AlkLEᵥEᵥᵢ | E₄*E₅ | E_{b} |
| 1135 | E₁ₐE₂E₃EᵢᵢE_{iib}E₄ | AlkLEᵥEᵥᵢ | E₄*E₅ | E_{b} |
| 1136 | E_{1b}E₂E₃EᵢᵢE_{iib}E₄ | AlkLEᵥEᵥᵢ | E₄*E₅ | E_{b} |
| 1137 | E₁ₐE₂Eᵢᵢᵢ | AlkLEᵥEᵥᵢ | E₄*E₅ | E_{b} |
| 1138 | E_{1b}E₂Eᵢᵢᵢ | AlkLEᵥEᵥᵢ | E₄*E₅ | E_{b} |
| 1139 | E₁ₐE₂E₃Eᵢᵢᵢ | AlkLEᵥEᵥᵢ | E₄*E₅ | E_{b} |
| 1140 | E_{1b}E₂E₃Eᵢᵢᵢ | AlkLEᵥEᵥᵢ | E₄*E₅ | E_{b} |
| 1141 | E₁ₐE₂EᵢᵢᵢE₄ | AlkLEᵥEᵥᵢ | E₄*E₅ | E_{b} |
| 1142 | E_{1b}E₂EᵢᵢᵢE₄ | AlkLEᵥEᵥᵢ | E₄*E₅ | E_{b} |
| 1143 | E₁ₐE₂E₃EᵢᵢᵢE₄ | AlkLEᵥEᵥᵢ | E₄*E₅ | E_{b} |
| 1144 | E_{1b}E₂E₃EᵢᵢᵢE₄ | AlkLEᵥEᵥᵢ | E₄*E₅ | E_{b} |
| 1145 | E₁ₐE₂Eᵢᵥ | AlkLEᵥEᵥᵢ | E₄*E₅ | E_{b} |
| 1146 | E_{1b}E₂Eᵢᵥ | AlkLEᵥEᵥᵢ | E₄*E₅ | E_{b} |
| 1147 | E₁ₐE₂E₃Eᵢᵥ | AlkLEᵥEᵥᵢ | E₄*E₅ | E_{b} |
| 1148 | E_{1b}E₂E₃Eᵢᵥ | AlkLEᵥEᵥᵢ | E₄*E₅ | E_{b} |
| 1149 | E₁ₐE₂EᵢᵥE₄ | AlkLEᵥEᵥᵢ | E₄*E₅ | E_{b} |
| 1150 | E_{1b}E₂EᵢᵥE₄ | AlkLEᵥEᵥᵢ | E₄*E₅ | E_{b} |
| 1151 | E₁ₐE₂E₃EᵢᵥE₄ | AlkLEᵥEᵥᵢ | E₄*E₅ | E_{b} |
| 1152 | E_{1b}E₂E₃EᵢᵥE₄ | AlkLEᵥEᵥᵢ | E₄*E₅ | E_{b} |
| 1153 | E₁ₐE₂ | EᵥEᵥᵢ | | E_{d} |
| 1154 | E_{1b}E₂ | EᵥEᵥᵢ | | E_{d} |
| 1155 | E₁ₐE₂E₃ | EᵥEᵥᵢ | | E_{d} |
| 1156 | E_{1b}E₂E₃ | EᵥEᵥᵢ | | E_{d} |
| 1157 | E₁ₐE₂Eᵢ | EᵥEᵥᵢ | | E_{d} |
| 1158 | E_{1b}E₂Eᵢ | EᵥEᵥᵢ | | E_{d} |
| 1159 | E₁ₐE₂Eᵢᵢ | EᵥEᵥᵢ | | E_{d} |
| 1160 | E_{1b}E₂Eᵢᵢ | EᵥEᵥᵢ | | E_{d} |
| 1161 | E₁ₐE₂EᵢᵢE_{iib} | EᵥEᵥᵢ | | E_{d} |
| 1162 | E_{1b}E₂EᵢᵢE_{iib} | EᵥEᵥᵢ | | E_{d} |
| 1163 | E₁ₐE₂E₃Eᵢ | EᵥEᵥᵢ | | E_{d} |
| 1164 | E_{1b}E₂E₃Eᵢ | EᵥEᵥᵢ | | E_{d} |
| 1165 | E₁ₐE₂E₃Eᵢᵢ | EᵥEᵥᵢ | | E_{d} |
| 1166 | E_{1b}E₂E₃Eᵢᵢ | EᵥEᵥᵢ | | E_{d} |
| 1167 | E₁ₐE₂E₃EᵢᵢE_{iib} | EᵥEᵥᵢ | | E_{d} |
| 1168 | E_{1b}E₂E₃EᵢᵢE_{iib} | EᵥEᵥᵢ | | E_{d} |
| 1169 | E₁ₐE₂E₄ | EᵥEᵥᵢ | | E_{d} |
| 1170 | E_{1b}E₂E₄ | EᵥEᵥᵢ | | E_{d} |
| 1171 | E₁ₐE₂E₃E₄ | EᵥEᵥᵢ | | E_{d} |
| 1172 | E_{1b}E₂E₃E₄ | EᵥEᵥᵢ | | E_{d} |
| 1173 | E₁ₐE₂EᵢE₄ | EᵥEᵥᵢ | | E_{d} |
| 1174 | E_{1b}E₂EᵢE₄ | EᵥEᵥᵢ | | E_{d} |
| 1175 | E₁ₐE₂EᵢᵢE₄ | EᵥEᵥᵢ | | E_{d} |
| 1176 | E_{1b}E₂EᵢᵢE₄ | EᵥEᵥᵢ | | E_{d} |
| 1177 | E₁ₐE₂EᵢᵢE_{iib}E₄ | EᵥEᵥᵢ | | E_{d} |
| 1178 | E_{1b}E₂EᵢᵢE_{iib}E₄ | EᵥEᵥᵢ | | E_{d} |
| 1179 | E₁ₐE₂E₃EᵢE₄ | EᵥEᵥᵢ | | E_{d} |
| 1180 | E_{1b}E₂E₃EᵢE₄ | EᵥEᵥᵢ | | E_{d} |
| 1181 | E₁ₐE₂E₃EᵢᵢE₄ | EᵥEᵥᵢ | | E_{d} |
| 1182 | E_{1b}E₂E₃EᵢᵢE₄ | EᵥEᵥᵢ | | E_{d} |
| 1183 | E₁ₐE₂E₃EᵢᵢE_{iib}E₄ | EᵥEᵥᵢ | | E_{d} |
| 1184 | E_{1b}E₂E₃EᵢᵢE_{iib}E₄ | EᵥEᵥᵢ | | E_{d} |
| 1185 | E₁ₐE₂Eᵢᵢᵢ | EᵥEᵥᵢ | | E_{d} |
| 1186 | E_{1b}E₂Eᵢᵢᵢ | EᵥEᵥᵢ | | E_{d} |
| 1187 | E₁ₐE₂E₃Eᵢᵢᵢ | EᵥEᵥᵢ | | E_{d} |
| 1188 | E_{1b}E₂E₃Eᵢᵢᵢ | EᵥEᵥᵢ | | E_{d} |
| 1189 | E₁ₐE₂EᵢᵢᵢE₄ | EᵥEᵥᵢ | | E_{d} |
| 1190 | E_{1b}E₂EᵢᵢᵢE₄ | EᵥEᵥᵢ | | E_{d} |
| 1191 | E₁ₐE₂E₃EᵢᵢᵢE₄ | EᵥEᵥᵢ | | E_{d} |
| 1192 | E_{1b}E₂E₃EᵢᵢᵢE₄ | EᵥEᵥᵢ | | E_{d} |
| 1193 | E₁ₐE₂Eᵢᵥ | EᵥEᵥᵢ | | E_{d} |
| 1194 | E_{1b}E₂Eᵢᵥ | EᵥEᵥᵢ | | E_{d} |
| 1195 | E₁ₐE₂E₃Eᵢᵥ | EᵥEᵥᵢ | | E_{d} |
| 1196 | E_{1b}E₂E₃Eᵢᵥ | EᵥEᵥᵢ | | E_{d} |
| 1197 | E₁ₐE₂EᵢᵥE₄ | EᵥEᵥᵢ | | E_{d} |
| 1198 | E_{1b}E₂EᵢᵥE₄ | EᵥEᵥᵢ | | E_{d} |
| 1199 | E₁ₐE₂E₃EᵢᵥE₄ | EᵥEᵥᵢ | | E_{d} |
| 1200 | E_{1b}E₂E₃EᵢᵥE₄ | EᵥEᵥᵢ | | E_{d} |
| 1201 | E₁ₐE₂ | EᵥEᵥᵢ | E₄* | E_{d} |
| 1202 | E_{1b}E₂ | EᵥEᵥᵢ | E₄* | E_{d} |
| 1203 | E₁ₐE₂E₃ | EᵥEᵥᵢ | E₄* | E_{d} |
| 1204 | E_{1b}E₂E₃ | EᵥEᵥᵢ | E₄* | E_{d} |
| 1205 | E₁ₐE₂Eᵢ | EᵥEᵥᵢ | E₄* | E_{d} |
| 1206 | E_{1b}E₂Eᵢ | EᵥEᵥᵢ | E₄* | E_{d} |
| 1207 | E₁ₐE₂Eᵢᵢ | EᵥEᵥᵢ | E₄* | E_{d} |
| 1208 | E_{1b}E₂Eᵢᵢ | EᵥEᵥᵢ | E₄* | E_{d} |
| 1209 | E₁ₐE₂EᵢᵢE_{iib} | EᵥEᵥᵢ | E₄* | E_{d} |
| 1210 | E_{1b}E₂EᵢᵢE_{iib} | EᵥEᵥᵢ | E₄* | E_{d} |
| 1211 | E₁ₐE₂E₃Eᵢ | EᵥEᵥᵢ | E₄* | E_{d} |
| 1212 | E_{1b}E₂E₃Eᵢ | EᵥEᵥᵢ | E₄* | E_{d} |
| 1213 | E₁ₐE₂E₃Eᵢᵢ | EᵥEᵥᵢ | E₄* | E_{d} |
| 1214 | E_{1b}E₂E₃Eᵢᵢ | EᵥEᵥᵢ | E₄* | E_{d} |
| 1215 | E₁ₐE₂E₃EᵢᵢE_{iib} | EᵥEᵥᵢ | E₄* | E_{d} |
| 1216 | E_{1b}E₂E₃EᵢᵢE_{iib} | EᵥEᵥᵢ | E₄* | E_{d} |
| 1217 | E₁ₐE₂E₄ | EᵥEᵥᵢ | E₄* | E_{d} |
| 1218 | E_{1b}E₂E₄ | EᵥEᵥᵢ | E₄* | E_{d} |
| 1219 | E₁ₐE₂E₃E₄ | EᵥEᵥᵢ | E₄* | E_{d} |
| 1220 | E_{1b}E₂E₃E₄ | EᵥEᵥᵢ | E₄* | E_{d} |
| 1221 | E₁ₐE₂EᵢE₄ | EᵥEᵥᵢ | E₄* | E_{d} |
| 1222 | E_{1b}E₂EᵢE₄ | EᵥEᵥᵢ | E₄* | E_{d} |
| 1223 | E₁ₐE₂EᵢᵢE₄ | EᵥEᵥᵢ | E₄* | E_{d} |
| 1224 | E_{1b}E₂EᵢᵢE₄ | EᵥEᵥᵢ | E₄* | E_{d} |
| 1225 | E₁ₐE₂EᵢᵢE_{iib}E₄ | EᵥEᵥᵢ | E₄* | E_{d} |
| 1226 | E_{1b}E₂EᵢᵢE_{iib}E₄ | EᵥEᵥᵢ | E₄* | E_{d} |
| 1227 | E₁ₐE₂E₃EᵢE₄ | EᵥEᵥᵢ | E₄* | E_{d} |
| 1228 | E_{1b}E₂E₃EᵢE₄ | EᵥEᵥᵢ | E₄* | E_{d} |
| 1229 | E₁ₐE₂E₃EᵢᵢE₄ | EᵥEᵥᵢ | E₄* | E_{d} |
| 1230 | E_{1b}E₂E₃EᵢᵢE₄ | EᵥEᵥᵢ | E₄* | E_{d} |
| 1231 | E₁ₐE₂E₃EᵢᵢE_{iib}E₄ | EᵥEᵥᵢ | E₄* | E_{d} |
| 1232 | E_{1b}E₂E₃EᵢᵢE_{iib}E₄ | EᵥEᵥᵢ | E₄* | E_{d} |
| 1233 | E₁ₐE₂Eᵢᵢᵢ | EᵥEᵥᵢ | E₄* | E_{d} |
| 1234 | E_{1b}E₂Eᵢᵢᵢ | EᵥEᵥᵢ | E₄* | E_{d} |
| 1235 | E₁ₐE₂E₃Eᵢᵢᵢ | EᵥEᵥᵢ | E₄* | E_{d} |
| 1236 | E_{1b}E₂E₃Eᵢᵢᵢ | EᵥEᵥᵢ | E₄* | E_{d} |
| 1237 | E₁ₐE₂EᵢᵢᵢE₄ | EᵥEᵥᵢ | E₄* | E_{d} |
| 1238 | E_{1b}E₂EᵢᵢᵢE₄ | EᵥEᵥᵢ | E₄* | E_{d} |
| 1239 | E₁ₐE₂E₃EᵢᵢᵢE₄ | EᵥEᵥᵢ | E₄* | E_{d} |
| 1240 | E_{1b}E₂E₃EᵢᵢᵢE₄ | EᵥEᵥᵢ | E₄* | E_{d} |
| 1241 | E₁ₐE₂Eᵢᵥ | EᵥEᵥᵢ | E₄* | E_{d} |
| 1242 | E_{1b}E₂Eᵢᵥ | EᵥEᵥᵢ | E₄* | E_{d} |
| 1243 | E₁ₐE₂E₃Eᵢᵥ | EᵥEᵥᵢ | E₄* | E_{d} |
| 1244 | E_{1b}E₂E₃Eᵢᵥ | EᵥEᵥᵢ | E₄* | E_{d} |
| 1245 | E₁ₐE₂EᵢᵥE₄ | EᵥEᵥᵢ | E₄* | E_{d} |
| 1246 | E_{1b}E₂EᵢᵥE₄ | EᵥEᵥᵢ | E₄* | E_{d} |
| 1247 | E₁ₐE₂E₃EᵢᵥE₄ | EᵥEᵥᵢ | E₄* | E_{d} |
| 1248 | E_{1b}E₂E₃EᵢᵥE₄ | EᵥEᵥᵢ | E₄* | E_{d} |
| 1249 | E₁ₐE₂ | EᵥEᵥᵢ | E₅ | E_{d} |
| 1250 | E_{1b}E₂ | EᵥEᵥᵢ | E₅ | E_{d} |
| 1251 | E₁ₐE₂E₃ | EᵥEᵥᵢ | E₅ | E_{d} |
| 1252 | E_{1b}E₂E₃ | EᵥEᵥᵢ | E₅ | E_{d} |
| 1253 | E₁ₐE₂Eᵢ | EᵥEᵥᵢ | E₅ | E_{d} |
| 1254 | E_{1b}E₂Eᵢ | EᵥEᵥᵢ | E₅ | E_{d} |
| 1255 | E₁ₐE₂Eᵢᵢ | EᵥEᵥᵢ | E₅ | E_{d} |
| 1256 | E_{1b}E₂Eᵢᵢ | EᵥEᵥᵢ | E₅ | E_{d} |
| 1257 | E₁ₐE₂EᵢᵢE_{iib} | EᵥEᵥᵢ | E₅ | E_{d} |
| 1258 | E_{1b}E₂EᵢᵢE_{iib} | EᵥEᵥᵢ | E₅ | E_{d} |
| 1259 | E₁ₐE₂E₃Eᵢ | EᵥEᵥᵢ | E₅ | E_{d} |
| 1260 | E_{1b}E₂E₃Eᵢ | EᵥEᵥᵢ | E₅ | E_{d} |
| 1261 | E₁ₐE₂E₃Eᵢᵢ | EᵥEᵥᵢ | E₅ | E_{d} |
| 1262 | E_{1b}E₂E₃Eᵢᵢ | EᵥEᵥᵢ | E₅ | E_{d} |
| 1263 | E₁ₐE₂E₃EᵢᵢE_{iib} | EᵥEᵥᵢ | E₅ | E_{d} |
| 1264 | E_{1b}E₂E₃EᵢᵢE_{iib} | EᵥEᵥᵢ | E₅ | E_{d} |
| 1265 | E₁ₐE₂E₄ | EᵥEᵥᵢ | E₅ | E_{d} |
| 1266 | E_{1b}E₂E₄ | EᵥEᵥᵢ | E₅ | E_{d} |
| 1267 | E₁ₐE₂E₃E₄ | EᵥEᵥᵢ | E₅ | E_{d} |
| 1268 | E_{1b}E₂E₃E₄ | EᵥEᵥᵢ | E₅ | E_{d} |
| 1269 | E₁ₐE₂EᵢE₄ | EᵥEᵥᵢ | E₅ | E_{d} |
| 1270 | E_{1b}E₂EᵢE₄ | EᵥEᵥᵢ | E₅ | E_{d} |
| 1271 | E₁ₐE₂EᵢᵢE₄ | EᵥEᵥᵢ | E₅ | E_{d} |
| 1272 | E_{1b}E₂EᵢᵢE₄ | EᵥEᵥᵢ | E₅ | E_{d} |
| 1273 | E₁ₐE₂EᵢᵢE_{iib}E₄ | EᵥEᵥᵢ | E₅ | E_{d} |
| 1274 | E_{1b}E₂EᵢᵢE_{iib}E₄ | EᵥEᵥᵢ | E₅ | E_{d} |
| 1275 | E₁ₐE₂E₃EᵢE₄ | EᵥEᵥᵢ | E₅ | E_{d} |
| 1276 | E_{1b}E₂E₃EᵢE₄ | EᵥEᵥᵢ | E₅ | E_{d} |
| 1277 | E₁ₐE₂E₃EᵢᵢE₄ | EᵥEᵥᵢ | E₅ | E_{d} |
| 1278 | E_{1b}E₂E₃EᵢᵢE₄ | EᵥEᵥᵢ | E₅ | E_{d} |
| 1279 | E₁ₐE₂E₃EᵢᵢE_{iib}E₄ | EᵥEᵥᵢ | E₅ | E_{d} |
| 1280 | E_{1b}E₂E₃EᵢᵢE_{iib}E₄ | EᵥEᵥᵢ | E₅ | E_{d} |
| 1281 | E₁ₐE₂Eᵢᵢᵢ | EᵥEᵥᵢ | E₅ | E_{d} |
| 1282 | E_{1b}E₂Eᵢᵢᵢ | EᵥEᵥᵢ | E₅ | E_{d} |
| 1283 | E₁ₐE₂E₃Eᵢᵢᵢ | EᵥEᵥᵢ | E₅ | E_{d} |
| 1284 | E_{1b}E₂E₃Eᵢᵢᵢ | EᵥEᵥᵢ | E₅ | E_{d} |
| 1285 | E₁ₐE₂EᵢᵢᵢE₄ | EᵥEᵥᵢ | E₅ | E_{d} |
| 1286 | E_{1b}E₂EᵢᵢᵢE₄ | EᵥEᵥᵢ | E₅ | E_{d} |
| 1287 | E₁ₐE₂E₃EᵢᵢᵢE₄ | EᵥEᵥᵢ | E₅ | E_{d} |
| 1288 | E_{1b}E₂E₃EᵢᵢᵢE₄ | EᵥEᵥᵢ | E₅ | E_{d} |
| 1289 | E₁ₐE₂Eᵢᵥ | EᵥEᵥᵢ | E₅ | E_{d} |
| 1290 | E_{1b}E₂Eᵢᵥ | EᵥEᵥᵢ | E₅ | E_{d} |
| 1291 | E₁ₐE₂E₃Eᵢᵥ | EᵥEᵥᵢ | E₅ | E_{d} |
| 1292 | E_{1b}E₂E₃Eᵢᵥ | EᵥEᵥᵢ | E₅ | E_{d} |
| 1293 | E₁ₐE₂EᵢᵥE₄ | EᵥEᵥᵢ | E₅ | E_{d} |
| 1294 | E_{1b}E₂EᵢᵥE₄ | EᵥEᵥᵢ | E₅ | E_{d} |
| 1295 | E₁ₐE₂E₃EᵢᵥE₄ | EᵥEᵥᵢ | E₅ | E_{d} |
| 1296 | E_{1b}E₂E₃EᵢᵥE₄ | EᵥEᵥᵢ | E₅ | E_{d} |
| 1297 | E₁ₐE₂ | EᵥEᵥᵢ | E₄*E₅ | E_{d} |
| 1298 | E_{1b}E₂ | EᵥEᵥᵢ | E₄*E₅ | E_{d} |
| 1299 | E₁ₐE₂E₃ | EᵥEᵥᵢ | E₄*E₅ | E_{d} |
| 1300 | E_{1b}E₂E₃ | EᵥEᵥᵢ | E₄*E₅ | E_{d} |
| 1301 | E₁ₐE₂Eᵢ | EᵥEᵥᵢ | E₄*E₅ | E_{d} |
| 1302 | E_{1b}E₂Eᵢ | EᵥEᵥᵢ | E₄*E₅ | E_{d} |
| 1303 | E₁ₐE₂Eᵢᵢ | EᵥEᵥᵢ | E₄*E₅ | E_{d} |
| 1304 | E_{1b}E₂Eᵢᵢ | EᵥEᵥᵢ | E₄*E₅ | E_{d} |
| 1305 | E₁ₐE₂EᵢᵢE_{iib} | EᵥEᵥᵢ | E₄*E₅ | E_{d} |
| 1306 | E_{1b}E₂EᵢᵢE_{iib} | EᵥEᵥᵢ | E₄*E₅ | E_{d} |
| 1307 | E₁ₐE₂E₃Eᵢ | EᵥEᵥᵢ | E₄*E₅ | E_{d} |
| 1308 | E_{1b}E₂E₃Eᵢ | EᵥEᵥᵢ | E₄*E₅ | E_{d} |
| 1309 | E₁ₐE₂E₃Eᵢᵢ | EᵥEᵥᵢ | E₄*E₅ | E_{d} |
| 1310 | E_{1b}E₂E₃Eᵢᵢ | EᵥEᵥᵢ | E₄*E₅ | E_{d} |
| 1311 | E₁ₐE₂E₃EᵢᵢE_{iib} | EᵥEᵥᵢ | E₄*E₅ | E_{d} |
| 1312 | E_{1b}E₂E₃EᵢᵢE_{iib} | EᵥEᵥᵢ | E₄*E₅ | E_{d} |
| 1313 | E₁ₐE₂E₄ | EᵥEᵥᵢ | E₄*E₅ | E_{d} |
| 1314 | E_{1b}E₂E₄ | EᵥEᵥᵢ | E₄*E₅ | E_{d} |
| 1315 | E₁ₐE₂E₃E₄ | EᵥEᵥᵢ | E₄*E₅ | E_{d} |
| 1316 | E_{1b}E₂E₃E₄ | EᵥEᵥᵢ | E₄*E₅ | E_{d} |
| 1317 | E₁ₐE₂EᵢE₄ | EᵥEᵥᵢ | E₄*E₅ | E_{d} |
| 1318 | E_{1b}E₂EᵢE₄ | EᵥEᵥᵢ | E₄*E₅ | E_{d} |
| 1319 | E₁ₐE₂EᵢᵢE₄ | EᵥEᵥᵢ | E₄*E₅ | E_{d} |
| 1320 | E_{1b}E₂EᵢᵢE₄ | EᵥEᵥᵢ | E₄*E₅ | E_{d} |
| 1321 | E₁ₐE₂EᵢᵢE_{iib}E₄ | EᵥEᵥᵢ | E₄*E₅ | E_{d} |
| 1322 | E_{1b}E₂EᵢᵢE_{iib}E₄ | EᵥEᵥᵢ | E₄*E₅ | E_{d} |
| 1323 | E₁ₐE₂E₃EᵢE₄ | EᵥEᵥᵢ | E₄*E₅ | E_{d} |
| 1324 | E_{1b}E₂E₃EᵢE₄ | EᵥEᵥᵢ | E₄*E₅ | E_{d} |
| 1325 | E₁ₐE₂E₃EᵢᵢE₄ | EᵥEᵥᵢ | E₄*E₅ | E_{d} |
| 1326 | E_{1b}E₂E₃EᵢᵢE₄ | EᵥEᵥᵢ | E₄*E₅ | E_{d} |
| 1327 | E₁ₐE₂E₃EᵢᵢE_{iib}E₄ | EᵥEᵥᵢ | E₄*E₅ | E_{d} |
| 1328 | E_{1b}E₂E₃EᵢᵢE_{iib}E₄ | EᵥEᵥᵢ | E₄*E₅ | E_{d} |
| 1329 | E₁ₐE₂Eᵢᵢᵢ | EᵥEᵥᵢ | E₄*E₅ | E_{d} |
| 1330 | E_{1b}E₂Eᵢᵢᵢ | EᵥEᵥᵢ | E₄*E₅ | E_{d} |
| 1331 | E₁ₐE₂E₃Eᵢᵢᵢ | EᵥEᵥᵢ | E₄*E₅ | E_{d} |
| 1332 | E_{1b}E₂E₃Eᵢᵢᵢ | EᵥEᵥᵢ | E₄*E₅ | E_{d} |
| 1333 | E₁ₐE₂EᵢᵢᵢE₄ | EᵥEᵥᵢ | E₄*E₅ | E_{d} |
| 1334 | E_{1b}E₂EᵢᵢᵢE₄ | EᵥEᵥᵢ | E₄*E₅ | E_{d} |
| 1335 | E₁ₐE₂E₃EᵢᵢᵢE₄ | EᵥEᵥᵢ | E₄*E₅ | E_{d} |
| 1336 | E_{1b}E₂E₃EᵢᵢᵢE₄ | EᵥEᵥᵢ | E₄*E₅ | E_{d} |
| 1337 | E₁ₐE₂Eᵢᵥ | EᵥEᵥᵢ | E₄*E₅ | E_{d} |
| 1338 | E_{1b}E₂Eᵢᵥ | EᵥEᵥᵢ | E₄*E₅ | E_{d} |
| 1339 | E₁ₐE₂E₃Eᵢᵥ | EᵥEᵥᵢ | E₄*E₅ | E_{d} |
| 1340 | E_{1b}E₂E₃Eᵢᵥ | EᵥEᵥᵢ | E₄*E₅ | E_{d} |
| 1341 | E₁ₐE₂EᵢᵥE₄ | EᵥEᵥᵢ | E₄*E₅ | E_{d} |
| 1342 | E_{1b}E₂EᵢᵥE₄ | EᵥEᵥᵢ | E₄*E₅ | E_{d} |
| 1343 | E₁ₐE₂E₃EᵢᵥE₄ | EᵥEᵥᵢ | E₄*E₅ | E_{d} |
| 1344 | E_{1b}E₂E₃EᵢᵥE₄ | EᵥEᵥᵢ | E₄*E₅ | E_{d} |
| 1345 | E₁ₐE₂ | AlkLEᵥEᵥᵢ | | E_{d} |
| 1346 | E_{1b}E₂ | AlkLEᵥEᵥᵢ | | E_{d} |
| 1347 | E₁ₐE₂E₃ | AlkLEᵥEᵥᵢ | | E_{d} |
| 1348 | E_{1b}E₂E₃ | AlkLEᵥEᵥᵢ | | E_{d} |
| 1349 | E₁ₐE₂Eᵢ | AlkLEᵥEᵥᵢ | | E_{d} |
| 1350 | E_{1b}E₂Eᵢ | AlkLEᵥEᵥᵢ | | E_{d} |
| 1351 | E₁ₐE₂Eᵢᵢ | AlkLEᵥEᵥᵢ | | E_{d} |
| 1352 | E_{1b}E₂Eᵢᵢ | AlkLEᵥEᵥᵢ | | E_{d} |
| 1353 | E₁ₐE₂EᵢᵢE_{iib} | AlkLEᵥEᵥᵢ | | E_{d} |
| 1354 | E_{1b}E₂EᵢᵢE_{iib} | AlkLEᵥEᵥᵢ | | E_{d} |
| 1355 | E₁ₐE₂E₃Eᵢ | AlkLEᵥEᵥᵢ | | E_{d} |
| 1356 | E_{1b}E₂E₃Eᵢ | AlkLEᵥEᵥᵢ | | E_{d} |
| 1357 | E₁ₐE₂E₃Eᵢᵢ | AlkLEᵥEᵥᵢ | | E_{d} |
| 1358 | E_{1b}E₂E₃Eᵢᵢ | AlkLEᵥEᵥᵢ | | E_{d} |
| 1359 | E₁ₐE₂E₃EᵢᵢE_{iib} | AlkLEᵥEᵥᵢ | | E_{d} |
| 1360 | E_{1b}E₂E₃EᵢᵢE_{iib} | AlkLEᵥEᵥᵢ | | E_{d} |
| 1361 | E₁ₐE₂E₄ | AlkLEᵥEᵥᵢ | | E_{d} |
| 1362 | E_{1b}E₂E₄ | AlkLEᵥEᵥᵢ | | E_{d} |
| 1363 | E₁ₐE₂E₃E₄ | AlkLEᵥEᵥᵢ | | E_{d} |
| 1364 | E_{1b}E₂E₃E₄ | AlkLEᵥEᵥᵢ | | E_{d} |
| 1365 | E₁ₐE₂EᵢE₄ | AlkLEᵥEᵥᵢ | | E_{d} |
| 1366 | E_{1b}E₂EᵢE₄ | AlkLEᵥEᵥᵢ | | E_{d} |
| 1367 | E₁ₐE₂EᵢᵢE₄ | AlkLEᵥEᵥᵢ | | E_{d} |
| 1368 | E_{1b}E₂EᵢᵢE₄ | AlkLEᵥEᵥᵢ | | E_{d} |
| 1369 | E₁ₐE₂EᵢᵢE_{iib}E₄ | AlkLEᵥEᵥᵢ | | E_{d} |
| 1370 | E_{1b}E₂EᵢᵢE_{iib}E₄ | AlkLEᵥEᵥᵢ | | E_{d} |
| 1371 | E₁ₐE₂E₃EᵢE₄ | AlkLEᵥEᵥᵢ | | E_{d} |
| 1372 | E_{1b}E₂E₃EᵢE₄ | AlkLEᵥEᵥᵢ | | E_{d} |
| 1373 | E₁ₐE₂E₃EᵢᵢE₄ | AlkLEᵥEᵥᵢ | | E_{d} |
| 1374 | E_{1b}E₂E₃EᵢᵢE₄ | AlkLEᵥEᵥᵢ | | E_{d} |
| 1375 | E₁ₐE₂E₃EᵢᵢE_{iib}E₄ | AlkLEᵥEᵥᵢ | | E_{d} |
| 1376 | E_{1b}E₂E₃EᵢᵢE_{iib}E₄ | AlkLEᵥEᵥᵢ | | E_{d} |
| 1377 | E₁ₐE₂Eᵢᵢᵢ | AlkLEᵥEᵥᵢ | | E_{d} |
| 1378 | E_{1b}E₂Eᵢᵢᵢ | AlkLEᵥEᵥᵢ | | E_{d} |
| 1379 | E₁ₐE₂E₃Eᵢᵢᵢ | AlkLEᵥEᵥᵢ | | E_{d} |
| 1380 | E_{1b}E₂E₃Eᵢᵢᵢ | AlkLEᵥEᵥᵢ | | E_{d} |
| 1381 | E₁ₐE₂EᵢᵢᵢE₄ | AlkLEᵥEᵥᵢ | | E_{d} |
| 1382 | E_{1b}E₂EᵢᵢᵢE₄ | AlkLEᵥEᵥᵢ | | E_{d} |
| 1383 | E₁ₐE₂E₃EᵢᵢᵢE₄ | AlkLEᵥEᵥᵢ | | E_{d} |
| 1384 | E_{1b}E₂E₃EᵢᵢᵢE₄ | AlkLEᵥEᵥᵢ | | E_{d} |
| 1385 | E₁ₐE₂Eᵢᵥ | AlkLEᵥEᵥᵢ | | E_{d} |
| 1386 | E_{1b}E₂Eᵢᵥ | AlkLEᵥEᵥᵢ | | E_{d} |
| 1387 | E₁ₐE₂E₃Eᵢᵥ | AlkLEᵥEᵥᵢ | | E_{d} |
| 1388 | E_{1b}E₂E₃Eᵢᵥ | AlkLEᵥEᵥᵢ | | E_{d} |
| 1389 | E₁ₐE₂EᵢᵥE₄ | AlkLEᵥEᵥᵢ | | E_{d} |
| 1390 | E_{1b}E₂EᵢᵥE₄ | AlkLEᵥEᵥᵢ | | E_{d} |
| 1391 | E₁ₐE₂E₃EᵢᵥE₄ | AlkLEᵥEᵥᵢ | | E_{d} |
| 1392 | E_{1b}E₂E₃EᵢᵥE₄ | AlkLEᵥEᵥᵢ | | E_{d} |
| 1393 | E₁ₐE₂ | AlkLEᵥEᵥᵢ | E₄* | E_{d} |
| 1394 | E_{1b}E₂ | AlkLEᵥEᵥᵢ | E₄* | E_{d} |
| 1395 | E₁ₐE₂E₃ | AlkLEᵥEᵥᵢ | E₄* | E_{d} |
| 1396 | E_{1b}E₂E₃ | AlkLEᵥEᵥᵢ | E₄* | E_{d} |
| 1397 | E₁ₐE₂Eᵢ | AlkLEᵥEᵥᵢ | E₄* | E_{d} |
| 1398 | E_{1b}E₂Eᵢ | AlkLEᵥEᵥᵢ | E₄* | E_{d} |
| 1399 | E₁ₐE₂Eᵢᵢ | AlkLEᵥEᵥᵢ | E₄* | E_{d} |
| 1400 | E_{1b}E₂Eᵢᵢ | AlkLEᵥEᵥᵢ | E₄* | E_{d} |
| 1401 | E₁ₐE₂EᵢᵢE_{iib} | AlkLEᵥEᵥᵢ | E₄* | E_{d} |
| 1402 | E_{1b}E₂EᵢᵢE_{iib} | AlkLEᵥEᵥᵢ | E₄* | E_{d} |
| 1403 | E₁ₐE₂E₃Eᵢ | AlkLEᵥEᵥᵢ | E₄* | E_{d} |
| 1404 | E_{1b}E₂E₃Eᵢ | AlkLEᵥEᵥᵢ | E₄* | E_{d} |
| 1405 | E₁ₐE₂E₃Eᵢᵢ | AlkLEᵥEᵥᵢ | E₄* | E_{d} |
| 1406 | E_{1b}E₂E₃Eᵢᵢ | AlkLEᵥEᵥᵢ | E₄* | E_{d} |
| 1407 | E₁ₐE₂E₃EᵢᵢE_{iib} | AlkLEᵥEᵥᵢ | E₄* | E_{d} |
| 1408 | E_{1b}E₂E₃EᵢᵢE_{iib} | AlkLEᵥEᵥᵢ | E₄* | E_{d} |
| 1409 | E₁ₐE₂E₄ | AlkLEᵥEᵥᵢ | E₄* | E_{d} |
| 1410 | E_{1b}E₂E₄ | AlkLEᵥEᵥᵢ | E₄* | E_{d} |
| 1411 | E₁ₐE₂E₃E₄ | AlkLEᵥEᵥᵢ | E₄* | E_{d} |
| 1412 | E_{1b}E₂E₃E₄ | AlkLEᵥEᵥᵢ | E₄* | E_{d} |
| 1413 | E₁ₐE₂EᵢE₄ | AlkLEᵥEᵥᵢ | E₄* | E_{d} |
| 1414 | E_{1b}E₂EᵢE₄ | AlkLEᵥEᵥᵢ | E₄* | E_{d} |
| 1415 | E₁ₐE₂EᵢᵢE₄ | AlkLEᵥEᵥᵢ | E₄* | E_{d} |
| 1416 | E_{1b}E₂EᵢᵢE₄ | AlkLEᵥEᵥᵢ | E₄* | E_{d} |
| 1417 | E₁ₐE₂EᵢᵢE_{iib}E₄ | AlkLEᵥEᵥᵢ | E₄* | E_{d} |
| 1418 | E_{1b}E₂EᵢᵢE_{iib}E₄ | AlkLEᵥEᵥᵢ | E₄* | E_{d} |
| 1419 | E₁ₐE₂E₃EᵢE₄ | AlkLEᵥEᵥᵢ | E₄* | E_{d} |
| 1420 | E_{1b}E₂E₃EᵢE₄ | AlkLEᵥEᵥᵢ | E₄* | E_{d} |
| 1421 | E₁ₐE₂E₃EᵢᵢE₄ | AlkLEᵥEᵥᵢ | E₄* | E_{d} |
| 1422 | E_{1b}E₂E₃EᵢᵢE₄ | AlkLEᵥEᵥᵢ | E₄* | E_{d} |
| 1423 | E₁ₐE₂E₃EᵢᵢE_{iib}E₄ | AlkLEᵥEᵥᵢ | E₄* | E_{d} |
| 1424 | E_{1b}E₂E₃EᵢᵢE_{iib}E₄ | AlkLEᵥEᵥᵢ | E₄* | E_{d} |
| 1425 | E₁ₐE₂Eᵢᵢᵢ | AlkLEᵥEᵥᵢ | E₄* | E_{d} |
| 1426 | E_{1b}E₂Eᵢᵢᵢ | AlkLEᵥEᵥᵢ | E₄* | E_{d} |
| 1427 | E₁ₐE₂E₃Eᵢᵢᵢ | AlkLEᵥEᵥᵢ | E₄* | E_{d} |
| 1428 | E_{1b}E₂E₃Eᵢᵢᵢ | AlkLEᵥEᵥᵢ | E₄* | E_{d} |
| 1429 | E₁ₐE₂EᵢᵢᵢE₄ | AlkLEᵥEᵥᵢ | E₄* | E_{d} |
| 1430 | E_{1b}E₂EᵢᵢᵢE₄ | AlkLEᵥEᵥᵢ | E₄* | E_{d} |
| 1431 | E₁ₐE₂E₃EᵢᵢᵢE₄ | AlkLEᵥEᵥᵢ | E₄* | E_{d} |
| 1432 | E_{1b}E₂E₃EᵢᵢᵢE₄ | AlkLEᵥEᵥᵢ | E₄* | E_{d} |
| 1433 | E₁ₐE₂Eᵢᵥ | AlkLEᵥEᵥᵢ | E₄* | E_{d} |
| 1434 | E_{1b}E₂Eᵢᵥ | AlkLEᵥEᵥᵢ | E₄* | E_{d} |
| 1435 | E₁ₐE₂E₃Eᵢᵥ | AlkLEᵥEᵥᵢ | E₄* | E_{d} |
| 1436 | E_{1b}E₂E₃Eᵢᵥ | AlkLEᵥEᵥᵢ | E₄* | E_{d} |
| 1437 | E₁ₐE₂EᵢᵥE₄ | AlkLEᵥEᵥᵢ | E₄* | E_{d} |
| 1438 | E_{1b}E₂EᵢᵥE₄ | AlkLEᵥEᵥᵢ | E₄* | E_{d} |
| 1439 | E₁ₐE₂E₃EᵢᵥE₄ | AlkLEᵥEᵥᵢ | E₄* | E_{d} |
| 1440 | E_{1b}E₂E₃EᵢᵥE₄ | AlkLEᵥEᵥᵢ | E₄* | E_{d} |
| 1441 | E₁ₐE₂ | AlkLEᵥEᵥᵢ | E₅ | E_{d} |
| 1442 | E_{1b}E₂ | AlkLEᵥEᵥᵢ | E₅ | E_{d} |
| 1443 | E₁ₐE₂E₃ | AlkLEᵥEᵥᵢ | E₅ | E_{d} |
| 1444 | E_{1b}E₂E₃ | AlkLEᵥEᵥᵢ | E₅ | E_{d} |
| 1445 | E₁ₐE₂Eᵢ | AlkLEᵥEᵥᵢ | E₅ | E_{d} |
| 1446 | E_{1b}E₂Eᵢ | AlkLEᵥEᵥᵢ | E₅ | E_{d} |
| 1447 | E₁ₐE₂Eᵢᵢ | AlkLEᵥEᵥᵢ | E₅ | E_{d} |
| 1448 | E_{1b}E₂Eᵢᵢ | AlkLEᵥEᵥᵢ | E₅ | E_{d} |
| 1449 | E₁ₐE₂EᵢᵢE_{iib} | AlkLEᵥEᵥᵢ | E₅ | E_{d} |
| 1450 | E_{1b}E₂EᵢᵢE_{iib} | AlkLEᵥEᵥᵢ | E₅ | E_{d} |
| 1451 | E₁ₐE₂E₃Eᵢ | AlkLEᵥEᵥᵢ | E₅ | E_{d} |
| 1452 | E_{1b}E₂E₃Eᵢ | AlkLEᵥEᵥᵢ | E₅ | E_{d} |
| 1453 | E₁ₐE₂E₃Eᵢᵢ | AlkLEᵥEᵥᵢ | E₅ | E_{d} |
| 1454 | E_{1b}E₂E₃Eᵢᵢ | AlkLEᵥEᵥᵢ | E₅ | E_{d} |
| 1455 | E₁ₐE₂E₃EᵢᵢE_{iib} | AlkLEᵥEᵥᵢ | E₅ | E_{d} |
| 1456 | E_{1b}E₂E₃EᵢᵢE_{iib} | AlkLEᵥEᵥᵢ | E₅ | E_{d} |
| 1457 | E₁ₐE₂E₄ | AlkLEᵥEᵥᵢ | E₅ | E_{d} |
| 1458 | E_{1b}E₂E₄ | AlkLEᵥEᵥᵢ | E₅ | E_{d} |
| 1459 | E₁ₐE₂E₃E₄ | AlkLEᵥEᵥᵢ | E₅ | E_{d} |
| 1460 | E_{1b}E₂E₃E₄ | AlkLEᵥEᵥᵢ | E₅ | E_{d} |
| 1461 | E₁ₐE₂EᵢE₄ | AlkLEᵥEᵥᵢ | E₅ | E_{d} |
| 1462 | E_{1b}E₂EᵢE₄ | AlkLEᵥEᵥᵢ | E₅ | E_{d} |
| 1463 | E₁ₐE₂EᵢᵢE₄ | AlkLEᵥEᵥᵢ | E₅ | E_{d} |
| 1464 | E_{1b}E₂EᵢᵢE₄ | AlkLEᵥEᵥᵢ | E₅ | E_{d} |
| 1465 | E₁ₐE₂EᵢᵢE_{iib}E₄ | AlkLEᵥEᵥᵢ | E₅ | E_{d} |
| 1466 | E_{1b}E₂EᵢᵢE_{iib}E₄ | AlkLEᵥEᵥᵢ | E₅ | E_{d} |
| 1467 | E₁ₐE₂E₃EᵢE₄ | AlkLEᵥEᵥᵢ | E₅ | E_{d} |
| 1468 | E_{1b}E₂E₃EᵢE₄ | AlkLEᵥEᵥᵢ | E₅ | E_{d} |
| 1469 | E₁ₐE₂E₃EᵢᵢE₄ | AlkLEᵥEᵥᵢ | E₅ | E_{d} |
| 1470 | E_{1b}E₂E₃EᵢᵢE₄ | AlkLEᵥEᵥᵢ | E₅ | E_{d} |
| 1471 | E₁ₐE₂E₃EᵢᵢE_{iib}E₄ | AlkLEᵥEᵥᵢ | E₅ | E_{d} |
| 1472 | E_{1b}E₂E₃EᵢᵢE_{iib}E₄ | AlkLEᵥEᵥᵢ | E₅ | E_{d} |
| 1473 | E₁ₐE₂Eᵢᵢᵢ | AlkLEᵥEᵥᵢ | E₅ | E_{d} |
| 1474 | E_{1b}E₂Eᵢᵢᵢ | AlkLEᵥEᵥᵢ | E₅ | E_{d} |
| 1475 | E₁ₐE₂E₃Eᵢᵢᵢ | AlkLEᵥEᵥᵢ | E₅ | E_{d} |
| 1476 | E_{1b}E₂E₃Eᵢᵢᵢ | AlkLEᵥEᵥᵢ | E₅ | E_{d} |
| 1477 | E₁ₐE₂EᵢᵢᵢE₄ | AlkLEᵥEᵥᵢ | E₅ | E_{d} |
| 1478 | E_{1b}E₂EᵢᵢᵢE₄ | AlkLEᵥEᵥᵢ | E₅ | E_{d} |
| 1479 | E₁ₐE₂E₃EᵢᵢᵢE₄ | AlkLEᵥEᵥᵢ | E₅ | E_{d} |
| 1480 | E_{1b}E₂E₃EᵢᵢᵢE₄ | AlkLEᵥEᵥᵢ | E₅ | E_{d} |
| 1481 | E₁ₐE₂Eᵢᵥ | AlkLEᵥEᵥᵢ | E₅ | E_{d} |
| 1482 | E_{1b}E₂Eᵢᵥ | AlkLEᵥEᵥᵢ | E₅ | E_{d} |
| 1483 | E₁ₐE₂E₃Eᵢᵥ | AlkLEᵥEᵥᵢ | E₅ | E_{d} |
| 1484 | E_{1b}E₂E₃Eᵢᵥ | AlkLEᵥEᵥᵢ | E₅ | E_{d} |
| 1485 | E₁ₐE₂EᵢᵥE₄ | AlkLEᵥEᵥᵢ | E₅ | E_{d} |
| 1486 | E_{1b}E₂EᵢᵥE₄ | AlkLEᵥEᵥᵢ | E₅ | E_{d} |
| 1487 | E₁ₐE₂E₃EᵢᵥE₄ | AlkLEᵥEᵥᵢ | E₅ | E_{d} |
| 1488 | E_{1b}E₂E₃EᵢᵥE₄ | AlkLEᵥEᵥᵢ | E₅ | E_{d} |
| 1489 | E₁ₐE₂ | AlkLEᵥEᵥᵢ | E₄*E₅ | E_{d} |
| 1490 | E_{1b}E₂ | AlkLEᵥEᵥᵢ | E₄*E₅ | E_{d} |
| 1491 | E₁ₐE₂E₃ | AlkLEᵥEᵥᵢ | E₄*E₅ | E_{d} |
| 1492 | E_{1b}E₂E₃ | AlkLEᵥEᵥᵢ | E₄*E₅ | E_{d} |
| 1493 | E₁ₐE₂Eᵢ | AlkLEᵥEᵥᵢ | E₄*E₅ | E_{d} |
| 1494 | E_{1b}E₂Eᵢ | AlkLEᵥEᵥᵢ | E₄*E₅ | E_{d} |
| 1495 | E₁ₐE₂Eᵢᵢ | AlkLEᵥEᵥᵢ | E₄*E₅ | E_{d} |
| 1496 | E_{1b}E₂Eᵢᵢ | AlkLEᵥEᵥᵢ | E₄*E₅ | E_{d} |
| 1497 | E₁ₐE₂EᵢᵢE_{iib} | AlkLEᵥEᵥᵢ | E₄*E₅ | E_{d} |
| 1498 | E_{1b}E₂EᵢᵢE_{iib} | AlkLEᵥEᵥᵢ | E₄*E₅ | E_{d} |
| 1499 | E₁ₐE₂E₃Eᵢ | AlkLEᵥEᵥᵢ | E₄*E₅ | E_{d} |
| 1500 | E_{1b}E₂E₃Eᵢ | AlkLEᵥEᵥᵢ | E₄*E₅ | E_{d} |
| 1501 | E₁ₐE₂E₃Eᵢᵢ | AlkLEᵥEᵥᵢ | E₄*E₅ | E_{d} |
| 1502 | E_{1b}E₂E₃Eᵢᵢ | AlkLEᵥEᵥᵢ | E₄*E₅ | E_{d} |
| 1503 | E₁ₐE₂E₃EᵢᵢE_{iib} | AlkLEᵥEᵥᵢ | E₄*E₅ | E_{d} |
| 1504 | E_{1b}E₂E₃EᵢᵢE_{iib} | AlkLEᵥEᵥᵢ | E₄*E₅ | E_{d} |
| 1505 | E₁ₐE₂E₄ | AlkLEᵥEᵥᵢ | E₄*E₅ | E_{d} |
| 1506 | E_{1b}E₂E₄ | AlkLEᵥEᵥᵢ | E₄*E₅ | E_{d} |
| 1507 | E₁ₐE₂E₃E₄ | AlkLEᵥEᵥᵢ | E₄*E₅ | E_{d} |
| 1508 | E_{1b}E₂E₃E₄ | AlkLEᵥEᵥᵢ | E₄*E₅ | E_{d} |
| 1509 | E₁ₐE₂EᵢE₄ | AlkLEᵥEᵥᵢ | E₄*E₅ | E_{d} |
| 1510 | E_{1b}E₂EᵢE₄ | AlkLEᵥEᵥᵢ | E₄*E₅ | E_{d} |
| 1511 | E₁ₐE₂EᵢᵢE₄ | AlkLEᵥEᵥᵢ | E₄*E₅ | E_{d} |
| 1512 | E_{1b}E₂EᵢᵢE₄ | AlkLEᵥEᵥᵢ | E₄*E₅ | E_{d} |
| 1513 | E₁ₐE₂EᵢᵢE_{iib}E₄ | AlkLEᵥEᵥᵢ | E₄*E₅ | E_{d} |
| 1514 | E_{1b}E₂EᵢᵢE_{iib}E₄ | AlkLEᵥEᵥᵢ | E₄*E₅ | E_{d} |
| 1515 | E₁ₐE₂E₃EᵢE₄ | AlkLEᵥEᵥᵢ | E₄*E₅ | E_{d} |
| 1516 | E_{1b}E₂E₃EᵢE₄ | AlkLEᵥEᵥᵢ | E₄*E₅ | E_{d} |
| 1517 | E₁ₐE₂E₃EᵢᵢE₄ | AlkLEᵥEᵥᵢ | E₄*E₅ | E_{d} |
| 1518 | E_{1b}E₂E₃EᵢᵢE₄ | AlkLEᵥEᵥᵢ | E₄*E₅ | E_{d} |
| 1519 | E₁ₐE₂E₃EᵢᵢE_{iib}E₄ | AlkLEᵥEᵥᵢ | E₄*E₅ | E_{d} |
| 1520 | E_{1b}E₂E₃EᵢᵢE_{iib}E₄ | AlkLEᵥEᵥᵢ | E₄*E₅ | E_{d} |
| 1521 | E₁ₐE₂Eᵢᵢᵢ | AlkLEᵥEᵥᵢ | E₄*E₅ | E_{d} |
| 1522 | E_{1b}E₂Eᵢᵢᵢ | AlkLEᵥEᵥᵢ | E₄*E₅ | E_{d} |
| 1523 | E₁ₐE₂E₃Eᵢᵢᵢ | AlkLEᵥEᵥᵢ | E₄*E₅ | E_{d} |
| 1524 | E_{1b}E₂E₃Eᵢᵢᵢ | AlkLEᵥEᵥᵢ | E₄*E₅ | E_{d} |
| 1525 | E₁ₐE₂EᵢᵢᵢE₄ | AlkLEᵥEᵥᵢ | E₄*E₅ | E_{d} |
| 1526 | E_{1b}E₂EᵢᵢᵢE₄ | AlkLEᵥEᵥᵢ | E₄*E₅ | E_{d} |
| 1527 | E₁ₐE₂E₃EᵢᵢᵢE₄ | AlkLEᵥEᵥᵢ | E₄*E₅ | E_{d} |
| 1528 | E_{1b}E₂E₃EᵢᵢᵢE₄ | AlkLEᵥEᵥᵢ | E₄*E₅ | E_{d} |
| 1529 | E₁ₐE₂Eᵢᵥ | AlkLEᵥEᵥᵢ | E₄*E₅ | E_{d} |
| 1530 | E_{1b}E₂Eᵢᵥ | AlkLEᵥEᵥᵢ | E₄*E₅ | E_{d} |
| 1531 | E₁ₐE₂E₃Eᵢᵥ | AlkLEᵥEᵥᵢ | E₄*E₅ | E_{d} |
| 1532 | E_{1b}E₂E₃Eᵢᵥ | AlkLEᵥEᵥᵢ | E₄*E₅ | E_{d} |
| 1533 | E₁ₐE₂EᵢᵥE₄ | AlkLEᵥEᵥᵢ | E₄*E₅ | E_{d} |
| 1534 | E_{1b}E₂EᵢᵥE₄ | AlkLEᵥEᵥᵢ | E₄*E₅ | E_{d} |
| 1535 | E₁ₐE₂E₃EᵢᵥE₄ | AlkLEᵥEᵥᵢ | E₄*E₅ | E_{d} |
| 1536 | E_{1b}E₂E₃EᵢᵥE₄ | AlkLEᵥEᵥᵢ | E₄*E₅ | E_{d} |
| 1537 | E₁ₐE₂ | EᵥEᵥᵢ | | Eₑ |
| 1538 | E_{1b}E₂ | EᵥEᵥᵢ | | Eₑ |
| 1539 | E₁ₐE₂E₃ | EᵥEᵥᵢ | | Eₑ |
| 1540 | E_{1b}E₂E₃ | EᵥEᵥᵢ | | Eₑ |
| 1541 | E₁ₐE₂Eᵢ | EᵥEᵥᵢ | | Eₑ |
| 1542 | E_{1b}E₂Eᵢ | EᵥEᵥᵢ | | Eₑ |
| 1543 | E₁ₐE₂Eᵢᵢ | EᵥEᵥᵢ | | Eₑ |
| 1544 | E_{1b}E₂Eᵢᵢ | EᵥEᵥᵢ | | Eₑ |
| 1545 | E₁ₐE₂EᵢᵢE_{iib} | EᵥEᵥᵢ | | Eₑ |
| 1546 | E_{1b}E₂EᵢᵢE_{iib} | EᵥEᵥᵢ | | Eₑ |
| 1547 | E₁ₐE₂E₃Eᵢ | EᵥEᵥᵢ | | Eₑ |
| 1548 | E_{1b}E₂E₃Eᵢ | EᵥEᵥᵢ | | Eₑ |
| 1549 | E₁ₐE₂E₃Eᵢᵢ | EᵥEᵥᵢ | | Eₑ |
| 1550 | E_{1b}E₂E₃Eᵢᵢ | EᵥEᵥᵢ | | Eₑ |
| 1551 | E₁ₐE₂E₃EᵢᵢE_{iib} | EᵥEᵥᵢ | | Eₑ |
| 1552 | E_{1b}E₂E₃EᵢᵢE_{iib} | EᵥEᵥᵢ | | Eₑ |
| 1553 | E₁ₐE₂E₄ | EᵥEᵥᵢ | | Eₑ |
| 1554 | E_{1b}E₂E₄ | EᵥEᵥᵢ | | Eₑ |
| 1555 | E₁ₐE₂E₃E₄ | EᵥEᵥᵢ | | Eₑ |
| 1556 | E_{1b}E₂E₃E₄ | EᵥEᵥᵢ | | Eₑ |
| 1557 | E₁ₐE₂EᵢE₄ | EᵥEᵥᵢ | | Eₑ |
| 1558 | E_{1b}E₂EᵢE₄ | EᵥEᵥᵢ | | Eₑ |
| 1559 | E₁ₐE₂EᵢᵢE₄ | EᵥEᵥᵢ | | Eₑ |
| 1560 | E_{1b}E₂EᵢᵢE₄ | EᵥEᵥᵢ | | Eₑ |
| 1561 | E₁ₐE₂EᵢᵢE_{iib}E₄ | EᵥEᵥᵢ | | Eₑ |
| 1562 | E_{1b}E₂EᵢᵢE_{iib}E₄ | EᵥEᵥᵢ | | Eₑ |
| 1563 | E₁ₐE₂E₃EᵢE₄ | EᵥEᵥᵢ | | Eₑ |
| 1564 | E_{1b}E₂E₃EᵢE₄ | EᵥEᵥᵢ | | Eₑ |
| 1565 | E₁ₐE₂E₃EᵢᵢE₄ | EᵥEᵥᵢ | | Eₑ |
| 1566 | E_{1b}E₂E₃EᵢᵢE₄ | EᵥEᵥᵢ | | Eₑ |
| 1567 | E₁ₐE₂E₃EᵢᵢE_{iib}E₄ | EᵥEᵥᵢ | | Eₑ |
| 1568 | E_{1b}E₂E₃EᵢᵢE_{iib}E₄ | EᵥEᵥᵢ | | Eₑ |
| 1569 | E₁ₐE₂Eᵢᵢᵢ | EᵥEᵥᵢ | | Eₑ |
| 1570 | E_{1b}E₂Eᵢᵢᵢ | EᵥEᵥᵢ | | Eₑ |
| 1571 | E₁ₐE₂E₃Eᵢᵢᵢ | EᵥEᵥᵢ | | Eₑ |
| 1572 | E_{1b}E₂E₃Eᵢᵢᵢ | EᵥEᵥᵢ | | Eₑ |
| 1573 | E₁ₐE₂EᵢᵢᵢE₄ | EᵥEᵥᵢ | | Eₑ |
| 1574 | E_{1b}E₂EᵢᵢᵢE₄ | EᵥEᵥᵢ | | Eₑ |
| 1575 | E₁ₐE₂E₃EᵢᵢᵢE₄ | EᵥEᵥᵢ | | Eₑ |
| 1576 | E_{1b}E₂E₃EᵢᵢᵢE₄ | EᵥEᵥᵢ | | Eₑ |
| 1577 | E₁ₐE₂Eᵢᵥ | EᵥEᵥᵢ | | Eₑ |
| 1578 | E_{1b}E₂Eᵢᵥ | EᵥEᵥᵢ | | Eₑ |
| 1579 | E₁ₐE₂E₃Eᵢᵥ | EᵥEᵥᵢ | | Eₑ |
| 1580 | E_{1b}E₂E₃Eᵢᵥ | EᵥEᵥᵢ | | Eₑ |
| 1581 | E₁ₐE₂EᵢᵥE₄ | EᵥEᵥᵢ | | Eₑ |
| 1582 | E_{1b}E₂EᵢᵥE₄ | EᵥEᵥᵢ | | Eₑ |
| 1583 | E₁ₐE₂E₃EᵢᵥE₄ | EᵥEᵥᵢ | | Eₑ |
| 1584 | E_{1b}E₂E₃EᵢᵥE₄ | EᵥEᵥᵢ | | Eₑ |
| 1585 | E₁ₐE₂ | EᵥEᵥᵢ | E₄* | Eₑ |
| 1586 | E_{1b}E₂ | EᵥEᵥᵢ | E₄* | Eₑ |
| 1587 | E₁ₐE₂E₃ | EᵥEᵥᵢ | E₄* | Eₑ |
| 1588 | E_{1b}E₂E₃ | EᵥEᵥᵢ | E₄* | Eₑ |
| 1589 | E₁ₐE₂Eᵢ | EᵥEᵥᵢ | E₄* | Eₑ |
| 1590 | E_{1b}E₂Eᵢ | EᵥEᵥᵢ | E₄* | Eₑ |
| 1591 | E₁ₐE₂Eᵢᵢ | EᵥEᵥᵢ | E₄* | Eₑ |
| 1592 | E_{1b}E₂Eᵢᵢ | EᵥEᵥᵢ | E₄* | Eₑ |
| 1593 | E₁ₐE₂EᵢᵢE_{iib} | EᵥEᵥᵢ | E₄* | Eₑ |
| 1594 | E_{1b}E₂EᵢᵢE_{iib} | EᵥEᵥᵢ | E₄* | Eₑ |
| 1595 | E₁ₐE₂E₃Eᵢ | EᵥEᵥᵢ | E₄* | Eₑ |
| 1596 | E_{1b}E₂E₃Eᵢ | EᵥEᵥᵢ | E₄* | Eₑ |
| 1597 | E₁ₐE₂E₃Eᵢᵢ | EᵥEᵥᵢ | E₄* | Eₑ |
| 1598 | E_{1b}E₂E₃Eᵢᵢ | EᵥEᵥᵢ | E₄* | Eₑ |
| 1599 | E₁ₐE₂E₃EᵢᵢE_{iib} | EᵥEᵥᵢ | E₄* | Eₑ |
| 1600 | E_{1b}E₂E₃EᵢᵢE_{iib} | EᵥEᵥᵢ | E₄* | Eₑ |
| 1601 | E₁ₐE₂E₄ | EᵥEᵥᵢ | E₄* | Eₑ |
| 1602 | E_{1b}E₂E₄ | EᵥEᵥᵢ | E₄* | Eₑ |
| 1603 | E₁ₐE₂E₃E₄ | EᵥEᵥᵢ | E₄* | Eₑ |
| 1604 | E_{1b}E₂E₃E₄ | EᵥEᵥᵢ | E₄* | Eₑ |
| 1605 | E₁ₐE₂EᵢE₄ | EᵥEᵥᵢ | E₄* | Eₑ |
| 1606 | E_{1b}E₂EᵢE₄ | EᵥEᵥᵢ | E₄* | Eₑ |
| 1607 | E₁ₐE₂EᵢᵢE₄ | EᵥEᵥᵢ | E₄* | Eₑ |
| 1608 | E_{1b}E₂EᵢᵢE₄ | EᵥEᵥᵢ | E₄* | Eₑ |
| 1609 | E₁ₐE₂EᵢᵢE_{iib}E₄ | EᵥEᵥᵢ | E₄* | Eₑ |
| 1610 | E_{1b}E₂EᵢᵢE_{iib}E₄ | EᵥEᵥᵢ | E₄* | Eₑ |
| 1611 | E₁ₐE₂E₃EᵢE₄ | EᵥEᵥᵢ | E₄* | Eₑ |
| 1612 | E_{1b}E₂E₃EᵢE₄ | EᵥEᵥᵢ | E₄* | Eₑ |
| 1613 | E₁ₐE₂E₃EᵢᵢE₄ | EᵥEᵥᵢ | E₄* | Eₑ |
| 1614 | E_{1b}E₂E₃EᵢᵢE₄ | EᵥEᵥᵢ | E₄* | Eₑ |
| 1615 | E₁ₐE₂E₃EᵢᵢE_{iib}E₄ | EᵥEᵥᵢ | E₄* | Eₑ |
| 1616 | E_{1b}E₂E₃EᵢᵢE_{iib}E₄ | EᵥEᵥᵢ | E₄* | Eₑ |
| 1617 | E₁ₐE₂Eᵢᵢᵢ | EᵥEᵥᵢ | E₄* | Eₑ |
| 1618 | E_{1b}E₂Eᵢᵢᵢ | EᵥEᵥᵢ | E₄* | Eₑ |
| 1619 | E₁ₐE₂E₃Eᵢᵢᵢ | EᵥEᵥᵢ | E₄* | Eₑ |
| 1620 | E_{1b}E₂E₃Eᵢᵢᵢ | EᵥEᵥᵢ | E₄* | Eₑ |
| 1621 | E₁ₐE₂EᵢᵢᵢE₄ | EᵥEᵥᵢ | E₄* | Eₑ |
| 1622 | E_{1b}E₂EᵢᵢᵢE₄ | EᵥEᵥᵢ | E₄* | Eₑ |
| 1623 | E₁ₐE₂E₃EᵢᵢᵢE₄ | EᵥEᵥᵢ | E₄* | Eₑ |
| 1624 | E_{1b}E₂E₃EᵢᵢᵢE₄ | EᵥEᵥᵢ | E₄* | Eₑ |
| 1625 | E₁ₐE₂Eᵢᵥ | EᵥEᵥᵢ | E₄* | Eₑ |
| 1626 | E_{1b}E₂Eᵢᵥ | EᵥEᵥᵢ | E₄* | Eₑ |
| 1627 | E₁ₐE₂E₃Eᵢᵥ | EᵥEᵥᵢ | E₄* | Eₑ |
| 1628 | E_{1b}E₂E₃Eᵢᵥ | EᵥEᵥᵢ | E₄* | Eₑ |
| 1629 | E₁ₐE₂EᵢᵥE₄ | EᵥEᵥᵢ | E₄* | Eₑ |
| 1630 | E_{1b}E₂EᵢᵥE₄ | EᵥEᵥᵢ | E₄* | Eₑ |
| 1631 | E₁ₐE₂E₃EᵢᵥE₄ | EᵥEᵥᵢ | E₄* | Eₑ |
| 1632 | E_{1b}E₂E₃EᵢᵥE₄ | EᵥEᵥᵢ | E₄* | Eₑ |
| 1633 | E₁ₐE₂ | EᵥEᵥᵢ | E₅ | Eₑ |
| 1634 | E_{1b}E₂ | EᵥEᵥᵢ | E₅ | Eₑ |
| 1635 | E₁ₐE₂E₃ | EᵥEᵥᵢ | E₅ | Eₑ |
| 1636 | E_{1b}E₂E₃ | EᵥEᵥᵢ | E₅ | Eₑ |
| 1637 | E₁ₐE₂Eᵢ | EᵥEᵥᵢ | E₅ | Eₑ |
| 1638 | E_{1b}E₂Eᵢ | EᵥEᵥᵢ | E₅ | Eₑ |
| 1639 | E₁ₐE₂Eᵢᵢ | EᵥEᵥᵢ | E₅ | Eₑ |
| 1640 | E_{1b}E₂Eᵢᵢ | EᵥEᵥᵢ | E₅ | Eₑ |
| 1641 | E₁ₐE₂EᵢᵢE_{iib} | EᵥEᵥᵢ | E₅ | Eₑ |
| 1642 | E_{1b}E₂EᵢᵢE_{iib} | EᵥEᵥᵢ | E₅ | Eₑ |
| 1643 | E₁ₐE₂E₃Eᵢ | EᵥEᵥᵢ | E₅ | Eₑ |
| 1644 | E_{1b}E₂E₃Eᵢ | EᵥEᵥᵢ | E₅ | Eₑ |
| 1645 | E₁ₐE₂E₃Eᵢᵢ | EᵥEᵥᵢ | E₅ | Eₑ |
| 1646 | E_{1b}E₂E₃Eᵢᵢ | EᵥEᵥᵢ | E₅ | Eₑ |
| 1647 | E₁ₐE₂E₃EᵢᵢE_{iib} | EᵥEᵥᵢ | E₅ | Eₑ |
| 1648 | E_{1b}E₂E₃EᵢᵢE_{iib} | EᵥEᵥᵢ | E₅ | Eₑ |
| 1649 | E₁ₐE₂E₄ | EᵥEᵥᵢ | E₅ | Eₑ |
| 1650 | E_{1b}E₂E₄ | EᵥEᵥᵢ | E₅ | Eₑ |
| 1651 | E₁ₐE₂E₃E₄ | EᵥEᵥᵢ | E₅ | Eₑ |
| 1652 | E_{1b}E₂E₃E₄ | EᵥEᵥᵢ | E₅ | Eₑ |
| 1653 | E₁ₐE₂EᵢE₄ | EᵥEᵥᵢ | E₅ | Eₑ |
| 1654 | E_{1b}E₂EᵢE₄ | EᵥEᵥᵢ | E₅ | Eₑ |
| 1655 | E₁ₐE₂EᵢᵢE₄ | EᵥEᵥᵢ | E₅ | Eₑ |
| 1656 | E_{1b}E₂EᵢᵢE₄ | EᵥEᵥᵢ | E₅ | Eₑ |
| 1657 | E₁ₐE₂EᵢᵢE_{iib}E₄ | EᵥEᵥᵢ | E₅ | Eₑ |
| 1658 | E_{1b}E₂EᵢᵢE_{iib}E₄ | EᵥEᵥᵢ | E₅ | Eₑ |
| 1659 | E₁ₐE₂E₃EᵢE₄ | EᵥEᵥᵢ | E₅ | Eₑ |
| 1660 | E_{1b}E₂E₃EᵢE₄ | EᵥEᵥᵢ | E₅ | Eₑ |
| 1661 | E₁ₐE₂E₃EᵢᵢE₄ | EᵥEᵥᵢ | E₅ | Eₑ |
| 1662 | E_{1b}E₂E₃EᵢᵢE₄ | EᵥEᵥᵢ | E₅ | Eₑ |
| 1663 | E₁ₐE₂E₃EᵢᵢE_{iib}E₄ | EᵥEᵥᵢ | E₅ | Eₑ |
| 1664 | E_{1b}E₂E₃EᵢᵢE_{iib}E₄ | EᵥEᵥᵢ | E₅ | Eₑ |
| 1665 | E₁ₐE₂Eᵢᵢᵢ | EᵥEᵥᵢ | E₅ | Eₑ |
| 1666 | E_{1b}E₂Eᵢᵢᵢ | EᵥEᵥᵢ | E₅ | Eₑ |
| 1667 | E₁ₐE₂E₃Eᵢᵢᵢ | EᵥEᵥᵢ | E₅ | Eₑ |
| 1668 | E_{1b}E₂E₃Eᵢᵢᵢ | EᵥEᵥᵢ | E₅ | Eₑ |
| 1669 | E₁ₐE₂EᵢᵢᵢE₄ | EᵥEᵥᵢ | E₅ | Eₑ |
| 1670 | E_{1b}E₂EᵢᵢᵢE₄ | EᵥEᵥᵢ | E₅ | Eₑ |
| 1671 | E₁ₐE₂E₃EᵢᵢᵢE₄ | EᵥEᵥᵢ | E₅ | Eₑ |
| 1672 | E_{1b}E₂E₃EᵢᵢᵢE₄ | EᵥEᵥᵢ | E₅ | Eₑ |
| 1673 | E₁ₐE₂Eᵢᵥ | EᵥEᵥᵢ | E₅ | Eₑ |
| 1674 | E_{1b}E₂Eᵢᵥ | EᵥEᵥᵢ | E₅ | Eₑ |
| 1675 | E₁ₐE₂E₃Eᵢᵥ | EᵥEᵥᵢ | E₅ | Eₑ |
| 1676 | E_{1b}E₂E₃Eᵢᵥ | EᵥEᵥᵢ | E₅ | Eₑ |
| 1677 | E₁ₐE₂EᵢᵥE₄ | EᵥEᵥᵢ | E₅ | Eₑ |
| 1678 | E_{1b}E₂EᵢᵥE₄ | EᵥEᵥᵢ | E₅ | Eₑ |
| 1679 | E₁ₐE₂E₃EᵢᵥE₄ | EᵥEᵥᵢ | E₅ | Eₑ |
| 1680 | E_{1b}E₂E₃EᵢᵥE₄ | EᵥEᵥᵢ | E₅ | Eₑ |
| 1681 | E₁ₐE₂ | EᵥEᵥᵢ | E₄*E₅ | Eₑ |
| 1682 | E_{1b}E₂ | EᵥEᵥᵢ | E₄*E₅ | Eₑ |
| 1683 | E₁ₐE₂E₃ | EᵥEᵥᵢ | E₄*E₅ | Eₑ |
| 1684 | E_{1b}E₂E₃ | EᵥEᵥᵢ | E₄*E₅ | Eₑ |
| 1685 | E₁ₐE₂Eᵢ | EᵥEᵥᵢ | E₄*E₅ | Eₑ |
| 1686 | E_{1b}E₂Eᵢ | EᵥEᵥᵢ | E₄*E₅ | Eₑ |
| 1687 | E₁ₐE₂Eᵢᵢ | EᵥEᵥᵢ | E₄*E₅ | Eₑ |
| 1688 | E_{1b}E₂Eᵢᵢ | EᵥEᵥᵢ | E₄*E₅ | Eₑ |
| 1689 | E₁ₐE₂EᵢᵢE_{iib} | EᵥEᵥᵢ | E₄*E₅ | Eₑ |
| 1690 | E_{1b}E₂EᵢᵢE_{iib} | EᵥEᵥᵢ | E₄*E₅ | Eₑ |
| 1691 | E₁ₐE₂E₃Eᵢ | EᵥEᵥᵢ | E₄*E₅ | Eₑ |
| 1692 | E_{1b}E₂E₃Eᵢ | EᵥEᵥᵢ | E₄*E₅ | Eₑ |
| 1693 | E₁ₐE₂E₃Eᵢᵢ | EᵥEᵥᵢ | E₄*E₅ | Eₑ |
| 1694 | E_{1b}E₂E₃Eᵢᵢ | EᵥEᵥᵢ | E₄*E₅ | Eₑ |
| 1695 | E₁ₐE₂E₃EᵢᵢE_{iib} | EᵥEᵥᵢ | E₄*E₅ | Eₑ |
| 1696 | E_{1b}E₂E₃EᵢᵢE_{iib} | EᵥEᵥᵢ | E₄*E₅ | Eₑ |
| 1697 | E₁ₐE₂E₄ | EᵥEᵥᵢ | E₄*E₅ | Eₑ |
| 1698 | E_{1b}E₂E₄ | EᵥEᵥᵢ | E₄*E₅ | Eₑ |
| 1699 | E₁ₐE₂E₃E₄ | EᵥEᵥᵢ | E₄*E₅ | Eₑ |
| 1700 | E_{1b}E₂E₃E₄ | EᵥEᵥᵢ | E₄*E₅ | Eₑ |
| 1701 | E₁ₐE₂EᵢE₄ | EᵥEᵥᵢ | E₄*E₅ | Eₑ |
| 1702 | E_{1b}E₂EᵢE₄ | EᵥEᵥᵢ | E₄*E₅ | Eₑ |
| 1703 | E₁ₐE₂EᵢᵢE₄ | EᵥEᵥᵢ | E₄*E₅ | Eₑ |
| 1704 | E_{1b}E₂EᵢᵢE₄ | EᵥEᵥᵢ | E₄*E₅ | Eₑ |
| 1705 | E₁ₐE₂EᵢᵢE_{iib}E₄ | EᵥEᵥᵢ | E₄*E₅ | Eₑ |
| 1706 | E_{1b}E₂EᵢᵢE_{iib}E₄ | EᵥEᵥᵢ | E₄*E₅ | Eₑ |
| 1707 | E₁ₐE₂E₃EᵢE₄ | EᵥEᵥᵢ | E₄*E₅ | Eₑ |
| 1708 | E_{1b}E₂E₃EᵢE₄ | EᵥEᵥᵢ | E₄*E₅ | Eₑ |
| 1709 | E₁ₐE₂E₃EᵢᵢE₄ | EᵥEᵥᵢ | E₄*E₅ | Eₑ |
| 1710 | E_{1b}E₂E₃EᵢᵢE₄ | EᵥEᵥᵢ | E₄*E₅ | Eₑ |
| 1711 | E₁ₐE₂E₃EᵢᵢE_{iib}E₄ | EᵥEᵥᵢ | E₄*E₅ | Eₑ |
| 1712 | E_{1b}E₂E₃EᵢᵢE_{iib}E₄ | EᵥEᵥᵢ | E₄*E₅ | Eₑ |
| 1713 | E₁ₐE₂Eᵢᵢᵢ | EᵥEᵥᵢ | E₄*E₅ | Eₑ |
| 1714 | E_{1b}E₂Eᵢᵢᵢ | EᵥEᵥᵢ | E₄*E₅ | Eₑ |
| 1715 | E₁ₐE₂E₃Eᵢᵢᵢ | EᵥEᵥᵢ | E₄*E₅ | Eₑ |
| 1716 | E_{1b}E₂E₃Eᵢᵢᵢ | EᵥEᵥᵢ | E₄*E₅ | Eₑ |
| 1717 | E₁ₐE₂EᵢᵢᵢE₄ | EᵥEᵥᵢ | E₄*E₅ | Eₑ |
| 1718 | E_{1b}E₂EᵢᵢᵢE₄ | EᵥEᵥᵢ | E₄*E₅ | Eₑ |
| 1719 | E₁ₐE₂E₃EᵢᵢᵢE₄ | EᵥEᵥᵢ | E₄*E₅ | Eₑ |
| 1720 | E_{1b}E₂E₃EᵢᵢᵢE₄ | EᵥEᵥᵢ | E₄*E₅ | Eₑ |
| 1721 | E₁ₐE₂Eᵢᵥ | EᵥEᵥᵢ | E₄*E₅ | Eₑ |
| 1722 | E_{1b}E₂Eᵢᵥ | EᵥEᵥᵢ | E₄*E₅ | Eₑ |
| 1723 | E₁ₐE₂E₃Eᵢᵥ | EᵥEᵥᵢ | E₄*E₅ | Eₑ |
| 1724 | E_{1b}E₂E₃Eᵢᵥ | EᵥEᵥᵢ | E₄*E₅ | Eₑ |
| 1725 | E₁ₐE₂EᵢᵥE₄ | EᵥEᵥᵢ | E₄*E₅ | Eₑ |
| 1726 | E_{1b}E₂EᵢᵥE₄ | EᵥEᵥᵢ | E₄*E₅ | Eₑ |
| 1727 | E₁ₐE₂E₃EᵢᵥE₄ | EᵥEᵥᵢ | E₄*E₅ | Eₑ |
| 1728 | E_{1b}E₂E₃EᵢᵥE₄ | EᵥEᵥᵢ | E₄*E₅ | Eₑ |
| 1729 | E₁ₐE₂ | AlkLEᵥEᵥᵢ | | Eₑ |
| 1730 | E_{1b}E₂ | AlkLEᵥEᵥᵢ | | Eₑ |
| 1731 | E₁ₐE₂E₃ | AlkLEᵥEᵥᵢ | | Eₑ |
| 1732 | E_{1b}E₂E₃ | AlkLEᵥEᵥᵢ | | Eₑ |
| 1733 | E₁ₐE₂Eᵢ | AlkLEᵥEᵥᵢ | | Eₑ |
| 1734 | E_{1b}E₂Eᵢ | AlkLEᵥEᵥᵢ | | Eₑ |
| 1735 | E₁ₐE₂Eᵢᵢ | AlkLEᵥEᵥᵢ | | Eₑ |
| 1736 | E_{1b}E₂Eᵢᵢ | AlkLEᵥEᵥᵢ | | Eₑ |
| 1737 | E₁ₐE₂EᵢᵢE_{iib} | AlkLEᵥEᵥᵢ | | Eₑ |
| 1738 | E_{1b}E₂EᵢᵢE_{iib} | AlkLEᵥEᵥᵢ | | Eₑ |
| 1739 | E₁ₐE₂E₃Eᵢ | AlkLEᵥEᵥᵢ | | Eₑ |
| 1740 | E_{1b}E₂E₃Eᵢ | AlkLEᵥEᵥᵢ | | Eₑ |
| 1741 | E₁ₐE₂E₃Eᵢᵢ | AlkLEᵥEᵥᵢ | | Eₑ |
| 1742 | E_{1b}E₂E₃Eᵢᵢ | AlkLEᵥEᵥᵢ | | Eₑ |
| 1743 | E₁ₐE₂E₃EᵢᵢE_{iib} | AlkLEᵥEᵥᵢ | | Eₑ |
| 1744 | E_{1b}E₂E₃EᵢᵢE_{iib} | AlkLEᵥEᵥᵢ | | Eₑ |
| 1745 | E₁ₐE₂E₄ | AlkLEᵥEᵥᵢ | | Eₑ |
| 1746 | E_{1b}E₂E₄ | AlkLEᵥEᵥᵢ | | Eₑ |
| 1747 | E₁ₐE₂E₃E₄ | AlkLEᵥEᵥᵢ | | Eₑ |
| 1748 | E_{1b}E₂E₃E₄ | AlkLEᵥEᵥᵢ | | Eₑ |
| 1749 | E₁ₐE₂EᵢE₄ | AlkLEᵥEᵥᵢ | | Eₑ |
| 1750 | E_{1b}E₂EᵢE₄ | AlkLEᵥEᵥᵢ | | Eₑ |
| 1751 | E₁ₐE₂EᵢᵢE₄ | AlkLEᵥEᵥᵢ | | Eₑ |
| 1752 | E_{1b}E₂EᵢᵢE₄ | AlkLEᵥEᵥᵢ | | Eₑ |
| 1753 | E₁ₐE₂EᵢᵢE_{iib}E₄ | AlkLEᵥEᵥᵢ | | Eₑ |
| 1754 | E_{1b}E₂EᵢᵢE_{iib}E₄ | AlkLEᵥEᵥᵢ | | Eₑ |
| 1755 | E₁ₐE₂E₃EᵢE₄ | AlkLEᵥEᵥᵢ | | Eₑ |
| 1756 | E_{1b}E₂E₃EᵢE₄ | AlkLEᵥEᵥᵢ | | Eₑ |
| 1757 | E₁ₐE₂E₃EᵢᵢE₄ | AlkLEᵥEᵥᵢ | | Eₑ |
| 1758 | E_{1b}E₂E₃EᵢᵢE₄ | AlkLEᵥEᵥᵢ | | Eₑ |
| 1759 | E₁ₐE₂E₃EᵢᵢE_{iib}E₄ | AlkLEᵥEᵥᵢ | | Eₑ |
| 1760 | E_{1b}E₂E₃EᵢᵢE_{iib}E₄ | AlkLEᵥEᵥᵢ | | Eₑ |
| 1761 | E₁ₐE₂Eᵢᵢᵢ | AlkLEᵥEᵥᵢ | | Eₑ |
| 1762 | E_{1b}E₂Eᵢᵢᵢ | AlkLEᵥEᵥᵢ | | Eₑ |
| 1763 | E₁ₐE₂E₃Eᵢᵢᵢ | AlkLEᵥEᵥᵢ | | Eₑ |
| 1764 | E_{1b}E₂E₃Eᵢᵢᵢ | AlkLEᵥEᵥᵢ | | Eₑ |
| 1765 | E₁ₐE₂EᵢᵢᵢE₄ | AlkLEᵥEᵥᵢ | | Eₑ |
| 1766 | E_{1b}E₂EᵢᵢᵢE₄ | AlkLEᵥEᵥᵢ | | Eₑ |
| 1767 | E₁ₐE₂E₃EᵢᵢᵢE₄ | AlkLEᵥEᵥᵢ | | Eₑ |
| 1768 | E_{1b}E₂E₃EᵢᵢᵢE₄ | AlkLEᵥEᵥᵢ | | Eₑ |
| 1769 | E₁ₐE₂Eᵢᵥ | AlkLEᵥEᵥᵢ | | Eₑ |
| 1770 | E_{1b}E₂Eᵢᵥ | AlkLEᵥEᵥᵢ | | Eₑ |
| 1771 | E₁ₐE₂E₃Eᵢᵥ | AlkLEᵥEᵥᵢ | | Eₑ |
| 1772 | E_{1b}E₂E₃Eᵢᵥ | AlkLEᵥEᵥᵢ | | Eₑ |
| 1773 | E₁ₐE₂EᵢᵥE₄ | AlkLEᵥEᵥᵢ | | Eₑ |
| 1774 | E_{1b}E₂EᵢᵥE₄ | AlkLEᵥEᵥᵢ | | Eₑ |
| 1775 | E₁ₐE₂E₃EᵢᵥE₄ | AlkLEᵥEᵥᵢ | | Eₑ |
| 1776 | E_{1b}E₂E₃EᵢᵥE₄ | AlkLEᵥEᵥᵢ | | Eₑ |
| 1777 | E₁ₐE₂ | AlkLEᵥEᵥᵢ | E₄* | Eₑ |
| 1778 | E_{1b}E₂ | AlkLEᵥEᵥᵢ | E₄* | Eₑ |
| 1779 | E₁ₐE₂E₃ | AlkLEᵥEᵥᵢ | E₄* | Eₑ |
| 1780 | E_{1b}E₂E₃ | AlkLEᵥEᵥᵢ | E₄* | Eₑ |
| 1781 | E₁ₐE₂Eᵢ | AlkLEᵥEᵥᵢ | E₄* | Eₑ |
| 1782 | E_{1b}E₂Eᵢ | AlkLEᵥEᵥᵢ | E₄* | Eₑ |
| 1783 | E₁ₐE₂Eᵢᵢ | AlkLEᵥEᵥᵢ | E₄* | Eₑ |
| 1784 | E_{1b}E₂Eᵢᵢ | AlkLEᵥEᵥᵢ | E₄* | Eₑ |
| 1785 | E₁ₐE₂EᵢᵢE_{iib} | AlkLEᵥEᵥᵢ | E₄* | Eₑ |
| 1786 | E_{1b}E₂EᵢᵢE_{iib} | AlkLEᵥEᵥᵢ | E₄* | Eₑ |
| 1787 | E₁ₐE₂E₃Eᵢ | AlkLEᵥEᵥᵢ | E₄* | Eₑ |
| 1788 | E_{1b}E₂E₃Eᵢ | AlkLEᵥEᵥᵢ | E₄* | Eₑ |
| 1789 | E₁ₐE₂E₃Eᵢᵢ | AlkLEᵥEᵥᵢ | E₄* | Eₑ |
| 1790 | E_{1b}E₂E₃Eᵢᵢ | AlkLEᵥEᵥᵢ | E₄* | Eₑ |
| 1791 | E₁ₐE₂E₃EᵢᵢE_{iib} | AlkLEᵥEᵥᵢ | E₄* | Eₑ |
| 1792 | E_{1b}E₂E₃EᵢᵢE_{iib} | AlkLEᵥEᵥᵢ | E₄* | Eₑ |
| 1793 | E₁ₐE₂E₄ | AlkLEᵥEᵥᵢ | E₄* | Eₑ |
| 1794 | E_{1b}E₂E₄ | AlkLEᵥEᵥᵢ | E₄* | Eₑ |
| 1795 | E₁ₐE₂E₃E₄ | AlkLEᵥEᵥᵢ | E₄* | Eₑ |
| 1796 | E_{1b}E₂E₃E₄ | AlkLEᵥEᵥᵢ | E₄* | Eₑ |
| 1797 | E₁ₐE₂EᵢE₄ | AlkLEᵥEᵥᵢ | E₄* | Eₑ |
| 1798 | E_{1b}E₂EᵢE₄ | AlkLEᵥEᵥᵢ | E₄* | Eₑ |
| 1799 | E₁ₐE₂EᵢᵢE₄ | AlkLEᵥEᵥᵢ | E₄* | Eₑ |
| 1800 | E_{1b}E₂EᵢᵢE₄ | AlkLEᵥEᵥᵢ | E₄* | Eₑ |
| 1801 | E₁ₐE₂EᵢᵢE_{iib}E₄ | AlkLEᵥEᵥᵢ | E₄* | Eₑ |
| 1802 | E_{1b}E₂EᵢᵢE_{iib}E₄ | AlkLEᵥEᵥᵢ | E₄* | Eₑ |
| 1803 | E₁ₐE₂E₃EᵢE₄ | AlkLEᵥEᵥᵢ | E₄* | Eₑ |
| 1804 | E_{1b}E₂E₃EᵢE₄ | AlkLEᵥEᵥᵢ | E₄* | Eₑ |
| 1805 | E₁ₐE₂E₃EᵢᵢE₄ | AlkLEᵥEᵥᵢ | E₄* | Eₑ |
| 1806 | E_{1b}E₂E₃EᵢᵢE₄ | AlkLEᵥEᵥᵢ | E₄* | Eₑ |
| 1807 | E₁ₐE₂E₃EᵢᵢE_{iib}E₄ | AlkLEᵥEᵥᵢ | E₄* | Eₑ |
| 1808 | E_{1b}E₂E₃EᵢᵢE_{iib}E₄ | AlkLEᵥEᵥᵢ | E₄* | Eₑ |
| 1809 | E₁ₐE₂Eᵢᵢᵢ | AlkLEᵥEᵥᵢ | E₄* | Eₑ |
| 1810 | E_{1b}E₂Eᵢᵢᵢ | AlkLEᵥEᵥᵢ | E₄* | Eₑ |
| 1811 | E₁ₐE₂E₃Eᵢᵢᵢ | AlkLEᵥEᵥᵢ | E₄* | Eₑ |
| 1812 | E_{1b}E₂E₃Eᵢᵢᵢ | AlkLEᵥEᵥᵢ | E₄* | Eₑ |
| 1813 | E₁ₐE₂EᵢᵢᵢE₄ | AlkLEᵥEᵥᵢ | E₄* | Eₑ |
| 1814 | E_{1b}E₂EᵢᵢᵢE₄ | AlkLEᵥEᵥᵢ | E₄* | Eₑ |
| 1815 | E₁ₐE₂E₃EᵢᵢᵢE₄ | AlkLEᵥEᵥᵢ | E₄* | Eₑ |
| 1816 | E_{1b}E₂E₃EᵢᵢᵢE₄ | AlkLEᵥEᵥᵢ | E₄* | Eₑ |
| 1817 | E₁ₐE₂Eᵢᵥ | AlkLEᵥEᵥᵢ | E₄* | Eₑ |
| 1818 | E_{1b}E₂Eᵢᵥ | AlkLEᵥEᵥᵢ | E₄* | Eₑ |
| 1819 | E₁ₐE₂E₃Eᵢᵥ | AlkLEᵥEᵥᵢ | E₄* | Eₑ |
| 1820 | E_{1b}E₂E₃Eᵢᵥ | AlkLEᵥEᵥᵢ | E₄* | Eₑ |
| 1821 | E₁ₐE₂EᵢᵥE₄ | AlkLEᵥEᵥᵢ | E₄* | Eₑ |
| 1822 | E_{1b}E₂EᵢᵥE₄ | AlkLEᵥEᵥᵢ | E₄* | Eₑ |
| 1823 | E₁ₐE₂E₃EᵢᵥE₄ | AlkLEᵥEᵥᵢ | E₄* | Eₑ |
| 1824 | E_{1b}E₂E₃EᵢᵥE₄ | AlkLEᵥEᵥᵢ | E₄* | Eₑ |
| 1825 | E₁ₐE₂ | AlkLEᵥEᵥᵢ | E₅ | Eₑ |
| 1826 | E_{1b}E₂ | AlkLEᵥEᵥᵢ | E₅ | Eₑ |
| 1827 | E₁ₐE₂E₃ | AlkLEᵥEᵥᵢ | E₅ | Eₑ |
| 1828 | E_{1b}E₂E₃ | AlkLEᵥEᵥᵢ | E₅ | Eₑ |
| 1829 | E₁ₐE₂Eᵢ | AlkLEᵥEᵥᵢ | E₅ | Eₑ |
| 1830 | E_{1b}E₂Eᵢ | AlkLEᵥEᵥᵢ | E₅ | Eₑ |
| 1831 | E₁ₐE₂Eᵢᵢ | AlkLEᵥEᵥᵢ | E₅ | Eₑ |
| 1832 | E_{1b}E₂Eᵢᵢ | AlkLEᵥEᵥᵢ | E₅ | Eₑ |
| 1833 | E₁ₐE₂EᵢᵢE_{iib} | AlkLEᵥEᵥᵢ | E₅ | Eₑ |
| 1834 | E_{1b}E₂EᵢᵢE_{iib} | AlkLEᵥEᵥᵢ | E₅ | Eₑ |
| 1835 | E₁ₐE₂E₃Eᵢ | AlkLEᵥEᵥᵢ | E₅ | Eₑ |
| 1836 | E_{1b}E₂E₃Eᵢ | AlkLEᵥEᵥᵢ | E₅ | Eₑ |
| 1837 | E₁ₐE₂E₃Eᵢᵢ | AlkLEᵥEᵥᵢ | E₅ | Eₑ |
| 1838 | E_{1b}E₂E₃Eᵢᵢ | AlkLEᵥEᵥᵢ | E₅ | Eₑ |
| 1839 | E₁ₐE₂E₃EᵢᵢE_{iib} | AlkLEᵥEᵥᵢ | E₅ | Eₑ |
| 1840 | E_{1b}E₂E₃EᵢᵢE_{iib} | AlkLEᵥEᵥᵢ | E₅ | Eₑ |
| 1841 | E₁ₐE₂E₄ | AlkLEᵥEᵥᵢ | E₅ | Eₑ |
| 1842 | E_{1b}E₂E₄ | AlkLEᵥEᵥᵢ | E₅ | Eₑ |
| 1843 | E₁ₐE₂E₃E₄ | AlkLEᵥEᵥᵢ | E₅ | Eₑ |
| 1844 | E_{1b}E₂E₃E₄ | AlkLEᵥEᵥᵢ | E₅ | Eₑ |
| 1845 | E₁ₐE₂EᵢE₄ | AlkLEᵥEᵥᵢ | E₅ | Eₑ |
| 1846 | E_{1b}E₂EᵢE₄ | AlkLEᵥEᵥᵢ | E₅ | Eₑ |
| 1847 | E₁ₐE₂EᵢᵢE₄ | AlkLEᵥEᵥᵢ | E₅ | Eₑ |
| 1848 | E_{1b}E₂EᵢᵢE₄ | AlkLEᵥEᵥᵢ | E₅ | Eₑ |
| 1849 | E₁ₐE₂EᵢᵢE_{iib}E₄ | AlkLEᵥEᵥᵢ | E₅ | Eₑ |
| 1850 | E_{1b}E₂EᵢᵢE_{iib}E₄ | AlkLEᵥEᵥᵢ | E₅ | Eₑ |
| 1851 | E₁ₐE₂E₃EᵢE₄ | AlkLEᵥEᵥᵢ | E₅ | Eₑ |
| 1852 | E_{1b}E₂E₃EᵢE₄ | AlkLEᵥEᵥᵢ | E₅ | Eₑ |
| 1853 | E₁ₐE₂E₃EᵢᵢE₄ | AlkLEᵥEᵥᵢ | E₅ | Eₑ |
| 1854 | E_{1b}E₂E₃EᵢᵢE₄ | AlkLEᵥEᵥᵢ | E₅ | Eₑ |
| 1855 | E₁ₐE₂E₃EᵢᵢE_{iib}E₄ | AlkLEᵥEᵥᵢ | E₅ | Eₑ |
| 1856 | E_{1b}E₂E₃EᵢᵢE_{iib}E₄ | AlkLEᵥEᵥᵢ | E₅ | Eₑ |
| 1857 | E₁ₐE₂Eᵢᵢᵢ | AlkLEᵥEᵥᵢ | E₅ | Eₑ |
| 1858 | E_{1b}E₂Eᵢᵢᵢ | AlkLEᵥEᵥᵢ | E₅ | Eₑ |
| 1859 | E₁ₐE₂E₃Eᵢᵢᵢ | AlkLEᵥEᵥᵢ | E₅ | Eₑ |
| 1860 | E_{1b}E₂E₃Eᵢᵢᵢ | AlkLEᵥEᵥᵢ | E₅ | Eₑ |
| 1861 | E₁ₐE₂EᵢᵢᵢE₄ | AlkLEᵥEᵥᵢ | E₅ | Eₑ |
| 1862 | E_{1b}E₂EᵢᵢᵢE₄ | AlkLEᵥEᵥᵢ | E₅ | Eₑ |
| 1863 | E₁ₐE₂E₃EᵢᵢᵢE₄ | AlkLEᵥEᵥᵢ | E₅ | Eₑ |
| 1864 | E_{1b}E₂E₃EᵢᵢᵢE₄ | AlkLEᵥEᵥᵢ | E₅ | Eₑ |
| 1865 | E₁ₐE₂Eᵢᵥ | AlkLEᵥEᵥᵢ | E₅ | Eₑ |
| 1866 | E_{1b}E₂Eᵢᵥ | AlkLEᵥEᵥᵢ | E₅ | Eₑ |
| 1867 | E₁ₐE₂E₃Eᵢᵥ | AlkLEᵥEᵥᵢ | E₅ | Eₑ |
| 1868 | E_{1b}E₂E₃Eᵢᵥ | AlkLEᵥEᵥᵢ | E₅ | Eₑ |
| 1869 | E₁ₐE₂EᵢᵥE₄ | AlkLEᵥEᵥᵢ | E₅ | Eₑ |
| 1870 | E_{1b}E₂EᵢᵥE₄ | AlkLEᵥEᵥᵢ | E₅ | Eₑ |
| 1871 | E₁ₐE₂E₃EᵢᵥE₄ | AlkLEᵥEᵥᵢ | E₅ | Eₑ |
| 1872 | E_{1b}E₂E₃EᵢᵥE₄ | AlkLEᵥEᵥᵢ | E₅ | Eₑ |
| 1873 | E₁ₐE₂ | AlkLEᵥEᵥᵢ | E₄*E₅ | Eₑ |
| 1874 | E_{1b}E₂ | AlkLEᵥEᵥᵢ | E₄*E₅ | Eₑ |
| 1875 | E₁ₐE₂E₃ | AlkLEᵥEᵥᵢ | E₄*E₅ | Eₑ |
| 1876 | E_{1b}E₂E₃ | AlkLEᵥEᵥᵢ | E₄*E₅ | Eₑ |
| 1877 | E₁ₐE₂Eᵢ | AlkLEᵥEᵥᵢ | E₄*E₅ | Eₑ |
| 1878 | E_{1b}E₂Eᵢ | AlkLEᵥEᵥᵢ | E₄*E₅ | Eₑ |
| 1879 | E₁ₐE₂Eᵢᵢ | AlkLEᵥEᵥᵢ | E₄*E₅ | Eₑ |
| 1880 | E_{1b}E₂Eᵢᵢ | AlkLEᵥEᵥᵢ | E₄*E₅ | Eₑ |
| 1881 | E₁ₐE₂EᵢᵢE_{iib} | AlkLEᵥEᵥᵢ | E₄*E₅ | Eₑ |
| 1882 | E_{1b}E₂EᵢᵢE_{iib} | AlkLEᵥEᵥᵢ | E₄*E₅ | Eₑ |
| 1883 | E₁ₐE₂E₃Eᵢ | AlkLEᵥEᵥᵢ | E₄*E₅ | Eₑ |
| 1884 | E_{1b}E₂E₃Eᵢ | AlkLEᵥEᵥᵢ | E₄*E₅ | Eₑ |
| 1885 | E₁ₐE₂E₃Eᵢᵢ | AlkLEᵥEᵥᵢ | E₄*E₅ | Eₑ |
| 1886 | E_{1b}E₂E₃Eᵢᵢ | AlkLEᵥEᵥᵢ | E₄*E₅ | Eₑ |
| 1887 | E₁ₐE₂E₃EᵢᵢE_{iib} | AlkLEᵥEᵥᵢ | E₄*E₅ | Eₑ |
| 1888 | E_{1b}E₂E₃EᵢᵢE_{iib} | AlkLEᵥEᵥᵢ | E₄*E₅ | Eₑ |
| 1889 | E₁ₐE₂E₄ | AlkLEᵥEᵥᵢ | E₄*E₅ | Eₑ |
| 1890 | E_{1b}E₂E₄ | AlkLEᵥEᵥᵢ | E₄*E₅ | Eₑ |
| 1891 | E₁ₐE₂E₃E₄ | AlkLEᵥEᵥᵢ | E₄*E₅ | Eₑ |
| 1892 | E_{1b}E₂E₃E₄ | AlkLEᵥEᵥᵢ | E₄*E₅ | Eₑ |
| 1893 | E₁ₐE₂EᵢE₄ | AlkLEᵥEᵥᵢ | E₄*E₅ | Eₑ |
| 1894 | E_{1b}E₂EᵢE₄ | AlkLEᵥEᵥᵢ | E₄*E₅ | Eₑ |
| 1895 | E₁ₐE₂EᵢᵢE₄ | AlkLEᵥEᵥᵢ | E₄*E₅ | Eₑ |
| 1896 | E_{1b}E₂EᵢᵢE₄ | AlkLEᵥEᵥᵢ | E₄*E₅ | Eₑ |
| 1897 | E₁ₐE₂EᵢᵢE_{iib}E₄ | AlkLEᵥEᵥᵢ | E₄*E₅ | Eₑ |
| 1898 | E_{1b}E₂EᵢᵢE_{iib}E₄ | AlkLEᵥEᵥᵢ | E₄*E₅ | Eₑ |
| 1899 | E₁ₐE₂E₃EᵢE₄ | AlkLEᵥEᵥᵢ | E₄*E₅ | Eₑ |
| 1900 | E_{1b}E₂E₃EᵢE₄ | AlkLEᵥEᵥᵢ | E₄*E₅ | Eₑ |
| 1901 | E₁ₐE₂E₃EᵢᵢE₄ | AlkLEᵥEᵥᵢ | E₄*E₅ | Eₑ |
| 1902 | E_{1b}E₂E₃EᵢᵢE₄ | AlkLEᵥEᵥᵢ | E₄*E₅ | Eₑ |
| 1903 | E₁ₐE₂E₃EᵢᵢE_{iib}E₄ | AlkLEᵥEᵥᵢ | E₄*E₅ | Eₑ |
| 1904 | E_{1b}E₂E₃EᵢᵢE_{iib}E₄ | AlkLEᵥEᵥᵢ | E₄*E₅ | Eₑ |
| 1905 | E₁ₐE₂Eᵢᵢᵢ | AlkLEᵥEᵥᵢ | E₄*E₅ | Eₑ |
| 1906 | E_{1b}E₂Eᵢᵢᵢ | AlkLEᵥEᵥᵢ | E₄*E₅ | Eₑ |
| 1907 | E₁ₐE₂E₃Eᵢᵢᵢ | AlkLEᵥEᵥᵢ | E₄*E₅ | Eₑ |
| 1908 | E_{1b}E₂E₃Eᵢᵢᵢ | AlkLEᵥEᵥᵢ | E₄*E₅ | Eₑ |
| 1909 | E₁ₐE₂EᵢᵢᵢE₄ | AlkLEᵥEᵥᵢ | E₄*E₅ | Eₑ |
| 1910 | E_{1b}E₂EᵢᵢᵢE₄ | AlkLEᵥEᵥᵢ | E₄*E₅ | Eₑ |
| 1911 | E₁ₐE₂E₃EᵢᵢᵢE₄ | AlkLEᵥEᵥᵢ | E₄*E₅ | Eₑ |
| 1912 | E_{1b}E₂E₃EᵢᵢᵢE₄ | AlkLEᵥEᵥᵢ | E₄*E₅ | Eₑ |
| 1913 | E₁ₐE₂Eᵢᵥ | AlkLEᵥEᵥᵢ | E₄*E₅ | Eₑ |
| 1914 | E_{1b}E₂Eᵢᵥ | AlkLEᵥEᵥᵢ | E₄*E₅ | Eₑ |
| 1915 | E₁ₐE₂E₃Eᵢᵥ | AlkLEᵥEᵥᵢ | E₄*E₅ | Eₑ |
| 1916 | E_{1b}E₂E₃Eᵢᵥ | AlkLEᵥEᵥᵢ | E₄*E₅ | Eₑ |
| 1917 | E₁ₐE₂EᵢᵥE₄ | AlkLEᵥEᵥᵢ | E₄*E₅ | Eₑ |
| 1918 | E_{1b}E₂EᵢᵥE₄ | AlkLEᵥEᵥᵢ | E₄*E₅ | Eₑ |
| 1919 | E₁ₐE₂E₃EᵢᵥE₄ | AlkLEᵥEᵥᵢ | E₄*E₅ | Eₑ |
| 1920 | E_{1b}E₂E₃EᵢᵥE₄ | AlkLEᵥEᵥᵢ | E₄*E₅ | Eₑ |
| 1921 | E₁ₐE₂ | EᵥEᵥᵢ | | E_{f} |
| 1922 | E_{1b}E₂ | EᵥEᵥᵢ | | E_{f} |
| 1923 | E₁ₐE₂E₃ | EᵥEᵥᵢ | | E_{f} |
| 1924 | E_{1b}E₂E₃ | EᵥEᵥᵢ | | E_{f} |
| 1925 | E₁ₐE₂Eᵢ | EᵥEᵥᵢ | | E_{f} |
| 1926 | E_{1b}E₂Eᵢ | EᵥEᵥᵢ | | E_{f} |
| 1927 | E₁ₐE₂Eᵢᵢ | EᵥEᵥᵢ | | E_{f} |
| 1928 | E_{1b}E₂Eᵢᵢ | EᵥEᵥᵢ | | E_{f} |
| 1929 | E₁ₐE₂EᵢᵢE_{iib} | EᵥEᵥᵢ | | E_{f} |
| 1930 | E_{1b}E₂EᵢᵢE_{iib} | EᵥEᵥᵢ | | E_{f} |
| 1931 | E₁ₐE₂E₃Eᵢ | EᵥEᵥᵢ | | E_{f} |
| 1932 | E_{1b}E₂E₃Eᵢ | EᵥEᵥᵢ | | E_{f} |
| 1933 | E₁ₐE₂E₃Eᵢᵢ | EᵥEᵥᵢ | | E_{f} |
| 1934 | E_{1b}E₂E₃Eᵢᵢ | EᵥEᵥᵢ | | E_{f} |
| 1935 | E₁ₐE₂E₃EᵢᵢE_{iib} | EᵥEᵥᵢ | | E_{f} |
| 1936 | E_{1b}E₂E₃EᵢᵢE_{iib} | EᵥEᵥᵢ | | E_{f} |
| 1937 | E₁ₐE₂E₄ | EᵥEᵥᵢ | | E_{f} |
| 1938 | E_{1b}E₂E₄ | EᵥEᵥᵢ | | E_{f} |
| 1939 | E₁ₐE₂E₃E₄ | EᵥEᵥᵢ | | E_{f} |
| 1940 | E_{1b}E₂E₃E₄ | EᵥEᵥᵢ | | E_{f} |
| 1941 | E₁ₐE₂EᵢE₄ | EᵥEᵥᵢ | | E_{f} |
| 1942 | E_{1b}E₂EᵢE₄ | EᵥEᵥᵢ | | E_{f} |
| 1943 | E₁ₐE₂EᵢᵢE₄ | EᵥEᵥᵢ | | E_{f} |
| 1944 | E_{1b}E₂EᵢᵢE₄ | EᵥEᵥᵢ | | E_{f} |
| 1945 | E₁ₐE₂EᵢᵢE_{iib}E₄ | EᵥEᵥᵢ | | E_{f} |
| 1946 | E_{1b}E₂EᵢᵢE_{iib}E₄ | EᵥEᵥᵢ | | E_{f} |
| 1947 | E₁ₐE₂E₃EᵢE₄ | EᵥEᵥᵢ | | E_{f} |
| 1948 | E_{1b}E₂E₃EᵢE₄ | EᵥEᵥᵢ | | E_{f} |
| 1949 | E₁ₐE₂E₃EᵢᵢE₄ | EᵥEᵥᵢ | | E_{f} |
| 1950 | E_{1b}E₂E₃EᵢᵢE₄ | EᵥEᵥᵢ | | E_{f} |
| 1951 | E₁ₐE₂E₃EᵢᵢE_{iib}E₄ | EᵥEᵥᵢ | | E_{f} |
| 1952 | E_{1b}E₂E₃EᵢᵢE_{iib}E₄ | EᵥEᵥᵢ | | E_{f} |
| 1953 | E₁ₐE₂Eᵢᵢᵢ | EᵥEᵥᵢ | | E_{f} |
| 1954 | E_{1b}E₂Eᵢᵢᵢ | EᵥEᵥᵢ | | E_{f} |
| 1955 | E₁ₐE₂E₃Eᵢᵢᵢ | EᵥEᵥᵢ | | E_{f} |
| 1956 | E_{1b}E₂E₃Eᵢᵢᵢ | EᵥEᵥᵢ | | E_{f} |
| 1957 | E₁ₐE₂EᵢᵢᵢE₄ | EᵥEᵥᵢ | | E_{f} |
| 1958 | E_{1b}E₂EᵢᵢᵢE₄ | EᵥEᵥᵢ | | E_{f} |
| 1959 | E₁ₐE₂E₃EᵢᵢᵢE₄ | EᵥEᵥᵢ | | E_{f} |
| 1960 | E_{1b}E₂E₃EᵢᵢᵢE₄ | EᵥEᵥᵢ | | E_{f} |
| 1961 | E₁ₐE₂Eᵢᵥ | EᵥEᵥᵢ | | E_{f} |
| 1962 | E_{1b}E₂Eᵢᵥ | EᵥEᵥᵢ | | E_{f} |
| 1963 | E₁ₐE₂E₃Eᵢᵥ | EᵥEᵥᵢ | | E_{f} |
| 1964 | E_{1b}E₂E₃Eᵢᵥ | EᵥEᵥᵢ | | E_{f} |
| 1965 | E₁ₐE₂EᵢᵥE₄ | EᵥEᵥᵢ | | E_{f} |
| 1966 | E_{1b}E₂EᵢᵥE₄ | EᵥEᵥᵢ | | E_{f} |
| 1967 | E₁ₐE₂E₃EᵢᵥE₄ | EᵥEᵥᵢ | | E_{f} |
| 1968 | E_{1b}E₂E₃EᵢᵥE₄ | EᵥEᵥᵢ | | E_{f} |
| 1969 | E₁ₐE₂ | EᵥEᵥᵢ | E₄* | E_{f} |
| 1970 | E_{1b}E₂ | EᵥEᵥᵢ | E₄* | E_{f} |
| 1971 | E₁ₐE₂E₃ | EᵥEᵥᵢ | E₄* | E_{f} |
| 1972 | E_{1b}E₂E₃ | EᵥEᵥᵢ | E₄* | E_{f} |
| 1973 | E₁ₐE₂Eᵢ | EᵥEᵥᵢ | E₄* | E_{f} |
| 1974 | E_{1b}E₂Eᵢ | EᵥEᵥᵢ | E₄* | E_{f} |
| 1975 | E₁ₐE₂Eᵢᵢ | EᵥEᵥᵢ | E₄* | E_{f} |
| 1976 | E_{1b}E₂Eᵢᵢ | EᵥEᵥᵢ | E₄* | E_{f} |
| 1977 | E₁ₐE₂EᵢᵢE_{iib} | EᵥEᵥᵢ | E₄* | E_{f} |
| 1978 | E_{1b}E₂EᵢᵢE_{iib} | EᵥEᵥᵢ | E₄* | E_{f} |
| 1979 | E₁ₐE₂E₃Eᵢ | EᵥEᵥᵢ | E₄* | E_{f} |
| 1980 | E_{1b}E₂E₃Eᵢ | EᵥEᵥᵢ | E₄* | E_{f} |
| 1981 | E₁ₐE₂E₃Eᵢᵢ | EᵥEᵥᵢ | E₄* | E_{f} |
| 1982 | E_{1b}E₂E₃Eᵢᵢ | EᵥEᵥᵢ | E₄* | E_{f} |
| 1983 | E₁ₐE₂E₃EᵢᵢE_{iib} | EᵥEᵥᵢ | E₄* | E_{f} |
| 1984 | E_{1b}E₂E₃EᵢᵢE_{iib} | EᵥEᵥᵢ | E₄* | E_{f} |
| 1985 | E₁ₐE₂E₄ | EᵥEᵥᵢ | E₄* | E_{f} |
| 1986 | E_{1b}E₂E₄ | EᵥEᵥᵢ | E₄* | E_{f} |
| 1987 | E₁ₐE₂E₃E₄ | EᵥEᵥᵢ | E₄* | E_{f} |
| 1988 | E_{1b}E₂E₃E₄ | EᵥEᵥᵢ | E₄* | E_{f} |
| 1989 | E₁ₐE₂EᵢE₄ | EᵥEᵥᵢ | E₄* | E_{f} |
| 1990 | E_{1b}E₂EᵢE₄ | EᵥEᵥᵢ | E₄* | E_{f} |
| 1991 | E₁ₐE₂EᵢᵢE₄ | EᵥEᵥᵢ | E₄* | E_{f} |
| 1992 | E_{1b}E₂EᵢᵢE₄ | EᵥEᵥᵢ | E₄* | E_{f} |
| 1993 | E₁ₐE₂EᵢᵢE_{iib}E₄ | EᵥEᵥᵢ | E₄* | E_{f} |
| 1994 | E_{1b}E₂EᵢᵢE_{iib}E₄ | EᵥEᵥᵢ | E₄* | E_{f} |
| 1995 | E₁ₐE₂E₃EᵢE₄ | EᵥEᵥᵢ | E₄* | E_{f} |
| 1996 | E_{1b}E₂E₃EᵢE₄ | EᵥEᵥᵢ | E₄* | E_{f} |
| 1997 | E₁ₐE₂E₃EᵢᵢE₄ | EᵥEᵥᵢ | E₄* | E_{f} |
| 1998 | E_{1b}E₂E₃EᵢᵢE₄ | EᵥEᵥᵢ | E₄* | E_{f} |
| 1999 | E₁ₐE₂E₃EᵢᵢE_{iib}E₄ | EᵥEᵥᵢ | E₄* | E_{f} |
| 2000 | E_{1b}E₂E₃EᵢᵢE_{iib}E₄ | EᵥEᵥᵢ | E₄* | E_{f} |
| 2001 | E₁ₐE₂Eᵢᵢᵢ | EᵥEᵥᵢ | E₄* | E_{f} |
| 2002 | E_{1b}E₂Eᵢᵢᵢ | EᵥEᵥᵢ | E₄* | E_{f} |
| 2003 | E₁ₐE₂E₃Eᵢᵢᵢ | EᵥEᵥᵢ | E₄* | E_{f} |
| 2004 | E_{1b}E₂E₃Eᵢᵢᵢ | EᵥEᵥᵢ | E₄* | E_{f} |
| 2005 | E₁ₐE₂EᵢᵢᵢE₄ | EᵥEᵥᵢ | E₄* | E_{f} |
| 2006 | E_{1b}E₂EᵢᵢᵢE₄ | EᵥEᵥᵢ | E₄* | E_{f} |
| 2007 | E₁ₐE₂E₃EᵢᵢᵢE₄ | EᵥEᵥᵢ | E₄* | E_{f} |
| 2008 | E_{1b}E₂E₃EᵢᵢᵢE₄ | EᵥEᵥᵢ | E₄* | E_{f} |
| 2009 | E₁ₐE₂Eᵢᵥ | EᵥEᵥᵢ | E₄* | E_{f} |
| 2010 | E_{1b}E₂Eᵢᵥ | EᵥEᵥᵢ | E₄* | E_{f} |
| 2011 | E₁ₐE₂E₃Eᵢᵥ | EᵥEᵥᵢ | E₄* | E_{f} |
| 2012 | E_{1b}E₂E₃Eᵢᵥ | EᵥEᵥᵢ | E₄* | E_{f} |
| 2013 | E₁ₐE₂EᵢᵥE₄ | EᵥEᵥᵢ | E₄* | E_{f} |
| 2014 | E_{1b}E₂EᵢᵥE₄ | EᵥEᵥᵢ | E₄* | E_{f} |
| 2015 | E₁ₐE₂E₃EᵢᵥE₄ | EᵥEᵥᵢ | E₄* | E_{f} |
| 2016 | E_{1b}E₂E₃EᵢᵥE₄ | EᵥEᵥᵢ | E₄* | E_{f} |
| 2017 | E₁ₐE₂ | EᵥEᵥᵢ | E₅ | E_{f} |
| 2018 | E_{1b}E₂ | EᵥEᵥᵢ | E₅ | E_{f} |
| 2019 | E₁ₐE₂E₃ | EᵥEᵥᵢ | E₅ | E_{f} |
| 2020 | E_{1b}E₂E₃ | EᵥEᵥᵢ | E₅ | E_{f} |
| 2021 | E₁ₐE₂Eᵢ | EᵥEᵥᵢ | E₅ | E_{f} |
| 2022 | E_{1b}E₂Eᵢ | EᵥEᵥᵢ | E₅ | E_{f} |
| 2023 | E₁ₐE₂Eᵢᵢ | EᵥEᵥᵢ | E₅ | E_{f} |
| 2024 | E_{1b}E₂Eᵢᵢ | EᵥEᵥᵢ | E₅ | E_{f} |
| 2025 | E₁ₐE₂EᵢᵢE_{iib} | EᵥEᵥᵢ | E₅ | E_{f} |
| 2026 | E_{1b}E₂EᵢᵢE_{iib} | EᵥEᵥᵢ | E₅ | E_{f} |
| 2027 | E₁ₐE₂E₃Eᵢ | EᵥEᵥᵢ | E₅ | E_{f} |
| 2028 | E_{1b}E₂E₃Eᵢ | EᵥEᵥᵢ | E₅ | E_{f} |
| 2029 | E₁ₐE₂E₃Eᵢᵢ | EᵥEᵥᵢ | E₅ | E_{f} |
| 2030 | E_{1b}E₂E₃Eᵢᵢ | EᵥEᵥᵢ | E₅ | E_{f} |
| 2031 | E₁ₐE₂E₃EᵢᵢE_{iib} | EᵥEᵥᵢ | E₅ | E_{f} |
| 2032 | E_{1b}E₂E₃EᵢᵢE_{iib} | EᵥEᵥᵢ | E₅ | E_{f} |
| 2033 | E₁ₐE₂E₄ | EᵥEᵥᵢ | E₅ | E_{f} |
| 2034 | E_{1b}E₂E₄ | EᵥEᵥᵢ | E₅ | E_{f} |
| 2035 | E₁ₐE₂E₃E₄ | EᵥEᵥᵢ | E₅ | E_{f} |
| 2036 | E_{1b}E₂E₃E₄ | EᵥEᵥᵢ | E₅ | E_{f} |
| 2037 | E₁ₐE₂EᵢE₄ | EᵥEᵥᵢ | E₅ | E_{f} |
| 2038 | E_{1b}E₂EᵢE₄ | EᵥEᵥᵢ | E₅ | E_{f} |
| 2039 | E₁ₐE₂EᵢᵢE₄ | EᵥEᵥᵢ | E₅ | E_{f} |
| 2040 | E_{1b}E₂EᵢᵢE₄ | EᵥEᵥᵢ | E₅ | E_{f} |
| 2041 | E₁ₐE₂EᵢᵢE_{iib}E₄ | EᵥEᵥᵢ | E₅ | E_{f} |
| 2042 | E_{1b}E₂EᵢᵢE_{iib}E₄ | EᵥEᵥᵢ | E₅ | E_{f} |
| 2043 | E₁ₐE₂E₃EᵢE₄ | EᵥEᵥᵢ | E₅ | E_{f} |
| 2044 | E_{1b}E₂E₃EᵢE₄ | EᵥEᵥᵢ | E₅ | E_{f} |
| 2045 | E₁ₐE₂E₃EᵢᵢE₄ | EᵥEᵥᵢ | E₅ | E_{f} |
| 2046 | E_{1b}E₂E₃EᵢᵢE₄ | EᵥEᵥᵢ | E₅ | E_{f} |
| 2047 | E₁ₐE₂E₃EᵢᵢE_{iib}E₄ | EᵥEᵥᵢ | E₅ | E_{f} |
| 2048 | E_{1b}E₂E₃EᵢᵢE_{iib}E₄ | EᵥEᵥᵢ | E₅ | E_{f} |
| 2049 | E₁ₐE₂Eᵢᵢᵢ | EᵥEᵥᵢ | E₅ | E_{f} |
| 2050 | E_{1b}E₂Eᵢᵢᵢ | EᵥEᵥᵢ | E₅ | E_{f} |
| 2051 | E₁ₐE₂E₃Eᵢᵢᵢ | EᵥEᵥᵢ | E₅ | E_{f} |
| 2052 | E_{1b}E₂E₃Eᵢᵢᵢ | EᵥEᵥᵢ | E₅ | E_{f} |
| 2053 | E₁ₐE₂EᵢᵢᵢE₄ | EᵥEᵥᵢ | E₅ | E_{f} |
| 2054 | E_{1b}E₂EᵢᵢᵢE₄ | EᵥEᵥᵢ | E₅ | E_{f} |
| 2055 | E₁ₐE₂E₃EᵢᵢᵢE₄ | EᵥEᵥᵢ | E₅ | E_{f} |
| 2056 | E_{1b}E₂E₃EᵢᵢᵢE₄ | EᵥEᵥᵢ | E₅ | E_{f} |
| 2057 | E₁ₐE₂Eᵢᵥ | EᵥEᵥᵢ | E₅ | E_{f} |
| 2058 | E_{1b}E₂Eᵢᵥ | EᵥEᵥᵢ | E₅ | E_{f} |
| 2059 | E₁ₐE₂E₃Eᵢᵥ | EᵥEᵥᵢ | E₅ | E_{f} |
| 2060 | E_{1b}E₂E₃Eᵢᵥ | EᵥEᵥᵢ | E₅ | E_{f} |
| 2061 | E₁ₐE₂EᵢᵥE₄ | EᵥEᵥᵢ | E₅ | E_{f} |
| 2062 | E_{1b}E₂EᵢᵥE₄ | EᵥEᵥᵢ | E₅ | E_{f} |
| 2063 | E₁ₐE₂E₃EᵢᵥE₄ | EᵥEᵥᵢ | E₅ | E_{f} |
| 2064 | E_{1b}E₂E₃EᵢᵥE₄ | EᵥEᵥᵢ | E₅ | E_{f} |
| 2065 | E₁ₐE₂ | EᵥEᵥᵢ | E₄*E₅ | E_{f} |
| 2066 | E_{1b}E₂ | EᵥEᵥᵢ | E₄*E₅ | E_{f} |
| 2067 | E₁ₐE₂E₃ | EᵥEᵥᵢ | E₄*E₅ | E_{f} |
| 2068 | E_{1b}E₂E₃ | EᵥEᵥᵢ | E₄*E₅ | E_{f} |
| 2069 | E₁ₐE₂Eᵢ | EᵥEᵥᵢ | E₄*E₅ | E_{f} |
| 2070 | E_{1b}E₂Eᵢ | EᵥEᵥᵢ | E₄*E₅ | E_{f} |
| 2071 | E₁ₐE₂Eᵢᵢ | EᵥEᵥᵢ | E₄*E₅ | E_{f} |
| 2072 | E_{1b}E₂Eᵢᵢ | EᵥEᵥᵢ | E₄*E₅ | E_{f} |
| 2073 | E₁ₐE₂EᵢᵢE_{iib} | EᵥEᵥᵢ | E₄*E₅ | E_{f} |
| 2074 | E_{1b}E₂EᵢᵢE_{iib} | EᵥEᵥᵢ | E₄*E₅ | E_{f} |
| 2075 | E₁ₐE₂E₃Eᵢ | EᵥEᵥᵢ | E₄*E₅ | E_{f} |
| 2076 | E_{1b}E₂E₃Eᵢ | EᵥEᵥᵢ | E₄*E₅ | E_{f} |
| 2077 | E₁ₐE₂E₃Eᵢᵢ | EᵥEᵥᵢ | E₄*E₅ | E_{f} |
| 2078 | E_{1b}E₂E₃Eᵢᵢ | EᵥEᵥᵢ | E₄*E₅ | E_{f} |
| 2079 | E₁ₐE₂E₃EᵢᵢE_{iib} | EᵥEᵥᵢ | E₄*E₅ | E_{f} |
| 2080 | E_{1b}E₂E₃EᵢᵢE_{iib} | EᵥEᵥᵢ | E₄*E₅ | E_{f} |
| 2081 | E₁ₐE₂E₄ | EᵥEᵥᵢ | E₄*E₅ | E_{f} |
| 2082 | E_{1b}E₂E₄ | EᵥEᵥᵢ | E₄*E₅ | E_{f} |
| 2083 | E₁ₐE₂E₃E₄ | EᵥEᵥᵢ | E₄*E₅ | E_{f} |
| 2084 | E_{1b}E₂E₃E₄ | EᵥEᵥᵢ | E₄*E₅ | E_{f} |
| 2085 | E₁ₐE₂EᵢE₄ | EᵥEᵥᵢ | E₄*E₅ | E_{f} |
| 2086 | E_{1b}E₂EᵢE₄ | EᵥEᵥᵢ | E₄*E₅ | E_{f} |
| 2087 | E₁ₐE₂EᵢᵢE₄ | EᵥEᵥᵢ | E₄*E₅ | E_{f} |
| 2088 | E_{1b}E₂EᵢᵢE₄ | EᵥEᵥᵢ | E₄*E₅ | E_{f} |
| 2089 | E₁ₐE₂EᵢᵢE_{iib}E₄ | EᵥEᵥᵢ | E₄*E₅ | E_{f} |
| 2090 | E_{1b}E₂EᵢᵢE_{iib}E₄ | EᵥEᵥᵢ | E₄*E₅ | E_{f} |
| 2091 | E₁ₐE₂E₃EᵢE₄ | EᵥEᵥᵢ | E₄*E₅ | E_{f} |
| 2092 | E_{1b}E₂E₃EᵢE₄ | EᵥEᵥᵢ | E₄*E₅ | E_{f} |
| 2093 | E₁ₐE₂E₃EᵢᵢE₄ | EᵥEᵥᵢ | E₄*E₅ | E_{f} |
| 2094 | E_{1b}E₂E₃EᵢᵢE₄ | EᵥEᵥᵢ | E₄*E₅ | E_{f} |
| 2095 | E₁ₐE₂E₃EᵢᵢE_{iib}E₄ | EᵥEᵥᵢ | E₄*E₅ | E_{f} |
| 2096 | E_{1b}E₂E₃EᵢᵢE_{iib}E₄ | EᵥEᵥᵢ | E₄*E₅ | E_{f} |
| 2097 | E₁ₐE₂Eᵢᵢᵢ | EᵥEᵥᵢ | E₄*E₅ | E_{f} |
| 2098 | E_{1b}E₂Eᵢᵢᵢ | EᵥEᵥᵢ | E₄*E₅ | E_{f} |
| 2099 | E₁ₐE₂E₃Eᵢᵢᵢ | EᵥEᵥᵢ | E₄*E₅ | E_{f} |
| 2100 | E_{1b}E₂E₃Eᵢᵢᵢ | EᵥEᵥᵢ | E₄*E₅ | E_{f} |
| 2101 | E₁ₐE₂EᵢᵢᵢE₄ | EᵥEᵥᵢ | E₄*E₅ | E_{f} |
| 2102 | E_{1b}E₂EᵢᵢᵢE₄ | EᵥEᵥᵢ | E₄*E₅ | E_{f} |
| 2103 | E₁ₐE₂E₃EᵢᵢᵢE₄ | EᵥEᵥᵢ | E₄*E₅ | E_{f} |
| 2104 | E_{1b}E₂E₃EᵢᵢᵢE₄ | EᵥEᵥᵢ | E₄*E₅ | E_{f} |
| 2105 | E₁ₐE₂Eᵢᵥ | EᵥEᵥᵢ | E₄*E₅ | E_{f} |
| 2106 | E_{1b}E₂Eᵢᵥ | EᵥEᵥᵢ | E₄*E₅ | E_{f} |
| 2107 | E₁ₐE₂E₃Eᵢᵥ | EᵥEᵥᵢ | E₄*E₅ | E_{f} |
| 2108 | E_{1b}E₂E₃Eᵢᵥ | EᵥEᵥᵢ | E₄*E₅ | E_{f} |
| 2109 | E₁ₐE₂EᵢᵥE₄ | EᵥEᵥᵢ | E₄*E₅ | E_{f} |
| 2110 | E_{1b}E₂EᵢᵥE₄ | EᵥEᵥᵢ | E₄*E₅ | E_{f} |
| 2111 | E₁ₐE₂E₃EᵢᵥE₄ | EᵥEᵥᵢ | E₄*E₅ | E_{f} |
| 2112 | E_{1b}E₂E₃EᵢᵥE₄ | EᵥEᵥᵢ | E₄*E₅ | E_{f} |
| 2113 | E₁ₐE₂ | AlkLEᵥEᵥᵢ | | E_{f} |
| 2114 | E_{1b}E₂ | AlkLEᵥEᵥᵢ | | E_{f} |
| 2115 | E₁ₐE₂E₃ | AlkLEᵥEᵥᵢ | | E_{f} |
| 2116 | E_{1b}E₂E₃ | AlkLEᵥEᵥᵢ | | E_{f} |
| 2117 | E₁ₐE₂Eᵢ | AlkLEᵥEᵥᵢ | | E_{f} |
| 2118 | E_{1b}E₂Eᵢ | AlkLEᵥEᵥᵢ | | E_{f} |
| 2119 | E₁ₐE₂Eᵢᵢ | AlkLEᵥEᵥᵢ | | E_{f} |
| 2120 | E_{1b}E₂Eᵢᵢ | AlkLEᵥEᵥᵢ | | E_{f} |
| 2121 | E₁ₐE₂EᵢᵢE_{iib} | AlkLEᵥEᵥᵢ | | E_{f} |
| 2122 | E_{1b}E₂EᵢᵢE_{iib} | AlkLEᵥEᵥᵢ | | E_{f} |
| 2123 | E₁ₐE₂E₃Eᵢ | AlkLEᵥEᵥᵢ | | E_{f} |
| 2124 | E_{1b}E₂E₃Eᵢ | AlkLEᵥEᵥᵢ | | E_{f} |
| 2125 | E₁ₐE₂E₃Eᵢᵢ | AlkLEᵥEᵥᵢ | | E_{f} |
| 2126 | E_{1b}E₂E₃Eᵢᵢ | AlkLEᵥEᵥᵢ | | E_{f} |
| 2127 | E₁ₐE₂E₃EᵢᵢE_{iib} | AlkLEᵥEᵥᵢ | | E_{f} |
| 2128 | E_{1b}E₂E₃EᵢᵢE_{iib} | AlkLEᵥEᵥᵢ | | E_{f} |
| 2129 | E₁ₐE₂E₄ | AlkLEᵥEᵥᵢ | | E_{f} |
| 2130 | E_{1b}E₂E₄ | AlkLEᵥEᵥᵢ | | E_{f} |
| 2131 | E₁ₐE₂E₃E₄ | AlkLEᵥEᵥᵢ | | E_{f} |
| 2132 | E_{1b}E₂E₃E₄ | AlkLEᵥEᵥᵢ | | E_{f} |
| 2133 | E₁ₐE₂EᵢE₄ | AlkLEᵥEᵥᵢ | | E_{f} |
| 2134 | E_{1b}E₂EᵢE₄ | AlkLEᵥEᵥᵢ | | E_{f} |
| 2135 | E₁ₐE₂EᵢᵢE₄ | AlkLEᵥEᵥᵢ | | E_{f} |
| 2136 | E_{1b}E₂EᵢᵢE₄ | AlkLEᵥEᵥᵢ | | E_{f} |
| 2137 | E₁ₐE₂EᵢᵢE_{iib}E₄ | AlkLEᵥEᵥᵢ | | E_{f} |
| 2138 | E_{1b}E₂EᵢᵢE_{iib}E₄ | AlkLEᵥEᵥᵢ | | E_{f} |
| 2139 | E₁ₐE₂E₃EᵢE₄ | AlkLEᵥEᵥᵢ | | E_{f} |
| 2140 | E_{1b}E₂E₃EᵢE₄ | AlkLEᵥEᵥᵢ | | E_{f} |
| 2141 | E₁ₐE₂E₃EᵢᵢE₄ | AlkLEᵥEᵥᵢ | | E_{f} |
| 2142 | E_{1b}E₂E₃EᵢᵢE₄ | AlkLEᵥEᵥᵢ | | E_{f} |
| 2143 | E₁ₐE₂E₃EᵢᵢE_{iib}E₄ | AlkLEᵥEᵥᵢ | | E_{f} |
| 2144 | E_{1b}E₂E₃EᵢᵢE_{iib}E₄ | AlkLEᵥEᵥᵢ | | E_{f} |
| 2145 | E₁ₐE₂Eᵢᵢᵢ | AlkLEᵥEᵥᵢ | | E_{f} |
| 2146 | E_{1b}E₂Eᵢᵢᵢ | AlkLEᵥEᵥᵢ | | E_{f} |
| 2147 | E₁ₐE₂E₃Eᵢᵢᵢ | AlkLEᵥEᵥᵢ | | E_{f} |
| 2148 | E_{1b}E₂E₃Eᵢᵢᵢ | AlkLEᵥEᵥᵢ | | E_{f} |
| 2149 | E₁ₐE₂EᵢᵢᵢE₄ | AlkLEᵥEᵥᵢ | | E_{f} |
| 2150 | E_{1b}E₂EᵢᵢᵢE₄ | AlkLEᵥEᵥᵢ | | E_{f} |
| 2151 | E₁ₐE₂E₃EᵢᵢᵢE₄ | AlkLEᵥEᵥᵢ | | E_{f} |
| 2152 | E_{1b}E₂E₃EᵢᵢᵢE₄ | AlkLEᵥEᵥᵢ | | E_{f} |
| 2153 | E₁ₐE₂Eᵢᵥ | AlkLEᵥEᵥᵢ | | E_{f} |
| 2154 | E_{1b}E₂Eᵢᵥ | AlkLEᵥEᵥᵢ | | E_{f} |
| 2155 | E₁ₐE₂E₃Eᵢᵥ | AlkLEᵥEᵥᵢ | | E_{f} |
| 2156 | E_{1b}E₂E₃Eᵢᵥ | AlkLEᵥEᵥᵢ | | E_{f} |
| 2157 | E₁ₐE₂EᵢᵥE₄ | AlkLEᵥEᵥᵢ | | E_{f} |
| 2158 | E_{1b}E₂EᵢᵥE₄ | AlkLEᵥEᵥᵢ | | E_{f} |
| 2159 | E₁ₐE₂E₃EᵢᵥE₄ | AlkLEᵥEᵥᵢ | | E_{f} |
| 2160 | E_{1b}E₂E₃EᵢᵥE₄ | AlkLEᵥEᵥᵢ | | E_{f} |
| 2161 | E₁ₐE₂ | AlkLEᵥEᵥᵢ | E₄* | E_{f} |
| 2162 | E_{1b}E₂ | AlkLEᵥEᵥᵢ | E₄* | E_{f} |
| 2163 | E₁ₐE₂E₃ | AlkLEᵥEᵥᵢ | E₄* | E_{f} |
| 2164 | E_{1b}E₂E₃ | AlkLEᵥEᵥᵢ | E₄* | E_{f} |
| 2165 | E₁ₐE₂Eᵢ | AlkLEᵥEᵥᵢ | E₄* | E_{f} |
| 2166 | E_{1b}E₂Eᵢ | AlkLEᵥEᵥᵢ | E₄* | E_{f} |
| 2167 | E₁ₐE₂Eᵢᵢ | AlkLEᵥEᵥᵢ | E₄* | E_{f} |
| 2168 | E_{1b}E₂Eᵢᵢ | AlkLEᵥEᵥᵢ | E₄* | E_{f} |
| 2169 | E₁ₐE₂EᵢᵢE_{iib} | AlkLEᵥEᵥᵢ | E₄* | E_{f} |
| 2170 | E_{1b}E₂EᵢᵢE_{iib} | AlkLEᵥEᵥᵢ | E₄* | E_{f} |
| 2171 | E₁ₐE₂E₃Eᵢ | AlkLEᵥEᵥᵢ | E₄* | E_{f} |
| 2172 | E_{1b}E₂E₃Eᵢ | AlkLEᵥEᵥᵢ | E₄* | E_{f} |
| 2173 | E₁ₐE₂E₃Eᵢᵢ | AlkLEᵥEᵥᵢ | E₄* | E_{f} |
| 2174 | E_{1b}E₂E₃Eᵢᵢ | AlkLEᵥEᵥᵢ | E₄* | E_{f} |
| 2175 | E₁ₐE₂E₃EᵢᵢE_{iib} | AlkLEᵥEᵥᵢ | E₄* | E_{f} |
| 2176 | E_{1b}E₂E₃EᵢᵢE_{iib} | AlkLEᵥEᵥᵢ | E₄* | E_{f} |
| 2177 | E₁ₐE₂E₄ | AlkLEᵥEᵥᵢ | E₄* | E_{f} |
| 2178 | E_{1b}E₂E₄ | AlkLEᵥEᵥᵢ | E₄* | E_{f} |
| 2179 | E₁ₐE₂E₃E₄ | AlkLEᵥEᵥᵢ | E₄* | E_{f} |
| 2180 | E_{1b}E₂E₃E₄ | AlkLEᵥEᵥᵢ | E₄* | E_{f} |
| 2181 | E₁ₐE₂EᵢE₄ | AlkLEᵥEᵥᵢ | E₄* | E_{f} |
| 2182 | E_{1b}E₂EᵢE₄ | AlkLEᵥEᵥᵢ | E₄* | E_{f} |
| 2183 | E₁ₐE₂EᵢᵢE₄ | AlkLEᵥEᵥᵢ | E₄* | E_{f} |
| 2184 | E_{1b}E₂EᵢᵢE₄ | AlkLEᵥEᵥᵢ | E₄* | E_{f} |
| 2185 | E₁ₐE₂EᵢᵢE_{iib}E₄ | AlkLEᵥEᵥᵢ | E₄* | E_{f} |
| 2186 | E_{1b}E₂EᵢᵢE_{iib}E₄ | AlkLEᵥEᵥᵢ | E₄* | E_{f} |
| 2187 | E₁ₐE₂E₃EᵢE₄ | AlkLEᵥEᵥᵢ | E₄* | E_{f} |
| 2188 | E_{1b}E₂E₃EᵢE₄ | AlkLEᵥEᵥᵢ | E₄* | E_{f} |
| 2189 | E₁ₐE₂E₃EᵢᵢE₄ | AlkLEᵥEᵥᵢ | E₄* | E_{f} |
| 2190 | E_{1b}E₂E₃EᵢᵢE₄ | AlkLEᵥEᵥᵢ | E₄* | E_{f} |
| 2191 | E₁ₐE₂E₃EᵢᵢE_{iib}E₄ | AlkLEᵥEᵥᵢ | E₄* | E_{f} |
| 2192 | E_{1b}E₂E₃EᵢᵢE_{iib}E₄ | AlkLEᵥEᵥᵢ | E₄* | E_{f} |
| 2193 | E₁ₐE₂Eᵢᵢᵢ | AlkLEᵥEᵥᵢ | E₄* | E_{f} |
| 2194 | E_{1b}E₂Eᵢᵢᵢ | AlkLEᵥEᵥᵢ | E₄* | E_{f} |
| 2195 | E₁ₐE₂E₃Eᵢᵢᵢ | AlkLEᵥEᵥᵢ | E₄* | E_{f} |
| 2196 | E_{1b}E₂E₃Eᵢᵢᵢ | AlkLEᵥEᵥᵢ | E₄* | E_{f} |
| 2197 | E₁ₐE₂EᵢᵢᵢE₄ | AlkLEᵥEᵥᵢ | E₄* | E_{f} |
| 2198 | E_{1b}E₂EᵢᵢᵢE₄ | AlkLEᵥEᵥᵢ | E₄* | E_{f} |
| 2199 | E₁ₐE₂E₃EᵢᵢᵢE₄ | AlkLEᵥEᵥᵢ | E₄* | E_{f} |
| 2200 | E_{1b}E₂E₃EᵢᵢᵢE₄ | AlkLEᵥEᵥᵢ | E₄* | E_{f} |
| 2201 | E₁ₐE₂Eᵢᵥ | AlkLEᵥEᵥᵢ | E₄* | E_{f} |
| 2202 | E_{1b}E₂Eᵢᵥ | AlkLEᵥEᵥᵢ | E₄* | E_{f} |
| 2203 | E₁ₐE₂E₃Eᵢᵥ | AlkLEᵥEᵥᵢ | E₄* | E_{f} |
| 2204 | E_{1b}E₂E₃Eᵢᵥ | AlkLEᵥEᵥᵢ | E₄* | E_{f} |
| 2205 | E₁ₐE₂EᵢᵥE₄ | AlkLEᵥEᵥᵢ | E₄* | E_{f} |
| 2206 | E_{1b}E₂EᵢᵥE₄ | AlkLEᵥEᵥᵢ | E₄* | E_{f} |
| 2207 | E₁ₐE₂E₃EᵢᵥE₄ | AlkLEᵥEᵥᵢ | E₄* | E_{f} |
| 2208 | E_{1b}E₂E₃EᵢᵥE₄ | AlkLEᵥEᵥᵢ | E₄* | E_{f} |
| 2209 | E₁ₐE₂ | AlkLEᵥEᵥᵢ | E₅ | E_{f} |
| 2210 | E_{1b}E₂ | AlkLEᵥEᵥᵢ | E₅ | E_{f} |
| 2211 | E₁ₐE₂E₃ | AlkLEᵥEᵥᵢ | E₅ | E_{f} |
| 2212 | E_{1b}E₂E₃ | AlkLEᵥEᵥᵢ | E₅ | E_{f} |
| 2213 | E₁ₐE₂Eᵢ | AlkLEᵥEᵥᵢ | E₅ | E_{f} |
| 2214 | E_{1b}E₂Eᵢ | AlkLEᵥEᵥᵢ | E₅ | E_{f} |
| 2215 | E₁ₐE₂Eᵢᵢ | AlkLEᵥEᵥᵢ | E₅ | E_{f} |
| 2216 | E_{1b}E₂Eᵢᵢ | AlkLEᵥEᵥᵢ | E₅ | E_{f} |
| 2217 | E₁ₐE₂EᵢᵢE_{iib} | AlkLEᵥEᵥᵢ | E₅ | E_{f} |
| 2218 | E_{1b}E₂EᵢᵢE_{iib} | AlkLEᵥEᵥᵢ | E₅ | E_{f} |
| 2219 | E₁ₐE₂E₃Eᵢ | AlkLEᵥEᵥᵢ | E₅ | E_{f} |
| 2220 | E_{1b}E₂E₃Eᵢ | AlkLEᵥEᵥᵢ | E₅ | E_{f} |
| 2221 | E₁ₐE₂E₃Eᵢᵢ | AlkLEᵥEᵥᵢ | E₅ | E_{f} |
| 2222 | E_{1b}E₂E₃Eᵢᵢ | AlkLEᵥEᵥᵢ | E₅ | E_{f} |
| 2223 | E₁ₐE₂E₃EᵢᵢE_{iib} | AlkLEᵥEᵥᵢ | E₅ | E_{f} |
| 2224 | E_{1b}E₂E₃EᵢᵢE_{iib} | AlkLEᵥEᵥᵢ | E₅ | E_{f} |
| 2225 | E₁ₐE₂E₄ | AlkLEᵥEᵥᵢ | E₅ | E_{f} |
| 2226 | E_{1b}E₂E₄ | AlkLEᵥEᵥᵢ | E₅ | E_{f} |
| 2227 | E₁ₐE₂E₃E₄ | AlkLEᵥEᵥᵢ | E₅ | E_{f} |
| 2228 | E_{1b}E₂E₃E₄ | AlkLEᵥEᵥᵢ | E₅ | E_{f} |
| 2229 | E₁ₐE₂EᵢE₄ | AlkLEᵥEᵥᵢ | E₅ | E_{f} |
| 2230 | E_{1b}E₂EᵢE₄ | AlkLEᵥEᵥᵢ | E₅ | E_{f} |
| 2231 | E₁ₐE₂EᵢᵢE₄ | AlkLEᵥEᵥᵢ | E₅ | E_{f} |
| 2232 | E_{1b}E₂EᵢᵢE₄ | AlkLEᵥEᵥᵢ | E₅ | E_{f} |
| 2233 | E₁ₐE₂EᵢᵢE_{iib}E₄ | AlkLEᵥEᵥᵢ | E₅ | E_{f} |
| 2234 | E_{1b}E₂EᵢᵢE_{iib}E₄ | AlkLEᵥEᵥᵢ | E₅ | E_{f} |
| 2235 | E₁ₐE₂E₃EᵢE₄ | AlkLEᵥEᵥᵢ | E₅ | E_{f} |
| 2236 | E_{1b}E₂E₃EᵢE₄ | AlkLEᵥEᵥᵢ | E₅ | E_{f} |
| 2237 | E₁ₐE₂E₃EᵢᵢE₄ | AlkLEᵥEᵥᵢ | E₅ | E_{f} |
| 2238 | E_{1b}E₂E₃EᵢᵢE₄ | AlkLEᵥEᵥᵢ | E₅ | E_{f} |
| 2239 | E₁ₐE₂E₃EᵢᵢE_{iib}E₄ | AlkLEᵥEᵥᵢ | E₅ | E_{f} |
| 2240 | E_{1b}E₂E₃EᵢᵢE_{iib}E₄ | AlkLEᵥEᵥᵢ | E₅ | E_{f} |
| 2241 | E₁ₐE₂Eᵢᵢᵢ | AlkLEᵥEᵥᵢ | E₅ | E_{f} |
| 2242 | E_{1b}E₂Eᵢᵢᵢ | AlkLEᵥEᵥᵢ | E₅ | E_{f} |
| 2243 | E₁ₐE₂E₃Eᵢᵢᵢ | AlkLEᵥEᵥᵢ | E₅ | E_{f} |
| 2244 | E_{1b}E₂E₃Eᵢᵢᵢ | AlkLEᵥEᵥᵢ | E₅ | E_{f} |
| 2245 | E₁ₐE₂EᵢᵢᵢE₄ | AlkLEᵥEᵥᵢ | E₅ | E_{f} |
| 2246 | E_{1b}E₂EᵢᵢᵢE₄ | AlkLEᵥEᵥᵢ | E₅ | E_{f} |
| 2247 | E₁ₐE₂E₃EᵢᵢᵢE₄ | AlkLEᵥEᵥᵢ | E₅ | E_{f} |
| 2248 | E_{1b}E₂E₃EᵢᵢᵢE₄ | AlkLEᵥEᵥᵢ | E₅ | E_{f} |
| 2249 | E₁ₐE₂Eᵢᵥ | AlkLEᵥEᵥᵢ | E₅ | E_{f} |
| 2250 | E_{1b}E₂Eᵢᵥ | AlkLEᵥEᵥᵢ | E₅ | E_{f} |
| 2251 | E₁ₐE₂E₃Eᵢᵥ | AlkLEᵥEᵥᵢ | E₅ | E_{f} |
| 2252 | E_{1b}E₂E₃Eᵢᵥ | AlkLEᵥEᵥᵢ | E₅ | E_{f} |
| 2253 | E₁ₐE₂EᵢᵥE₄ | AlkLEᵥEᵥᵢ | E₅ | E_{f} |
| 2254 | E_{1b}E₂EᵢᵥE₄ | AlkLEᵥEᵥᵢ | E₅ | E_{f} |
| 2255 | E₁ₐE₂E₃EᵢᵥE₄ | AlkLEᵥEᵥᵢ | E₅ | E_{f} |
| 2256 | E_{1b}E₂E₃EᵢᵥE₄ | AlkLEᵥEᵥᵢ | E₅ | E_{f} |
| 2257 | E₁ₐE₂ | AlkLEᵥEᵥᵢ | E₄*E₅ | E_{f} |
| 2258 | E_{1b}E₂ | AlkLEᵥEᵥᵢ | E₄*E₅ | E_{f} |
| 2259 | E₁ₐE₂E₃ | AlkLEᵥEᵥᵢ | E₄*E₅ | E_{f} |
| 2260 | E_{1b}E₂E₃ | AlkLEᵥEᵥᵢ | E₄*E₅ | E_{f} |
| 2261 | E₁ₐE₂Eᵢ | AlkLEᵥEᵥᵢ | E₄*E₅ | E_{f} |
| 2262 | E_{1b}E₂Eᵢ | AlkLEᵥEᵥᵢ | E₄*E₅ | E_{f} |
| 2263 | E₁ₐE₂Eᵢᵢ | AlkLEᵥEᵥᵢ | E₄*E₅ | E_{f} |
| 2264 | E_{1b}E₂Eᵢᵢ | AlkLEᵥEᵥᵢ | E₄*E₅ | E_{f} |
| 2265 | E₁ₐE₂EᵢᵢE_{iib} | AlkLEᵥEᵥᵢ | E₄*E₅ | E_{f} |
| 2266 | E_{1b}E₂EᵢᵢE_{iib} | AlkLEᵥEᵥᵢ | E₄*E₅ | E_{f} |
| 2267 | E₁ₐE₂E₃Eᵢ | AlkLEᵥEᵥᵢ | E₄*E₅ | E_{f} |
| 2268 | E_{1b}E₂E₃Eᵢ | AlkLEᵥEᵥᵢ | E₄*E₅ | E_{f} |
| 2269 | E₁ₐE₂E₃Eᵢᵢ | AlkLEᵥEᵥᵢ | E₄*E₅ | E_{f} |
| 2270 | E_{1b}E₂E₃Eᵢᵢ | AlkLEᵥEᵥᵢ | E₄*E₅ | E_{f} |
| 2271 | E₁ₐE₂E₃EᵢᵢE_{iib} | AlkLEᵥEᵥᵢ | E₄*E₅ | E_{f} |
| 2272 | E_{1b}E₂E₃EᵢᵢE_{iib} | AlkLEᵥEᵥᵢ | E₄*E₅ | E_{f} |
| 2273 | E₁ₐE₂E₄ | AlkLEᵥEᵥᵢ | E₄*E₅ | E_{f} |
| 2274 | E_{1b}E₂E₄ | AlkLEᵥEᵥᵢ | E₄*E₅ | E_{f} |
| 2275 | E₁ₐE₂E₃E₄ | AlkLEᵥEᵥᵢ | E₄*E₅ | E_{f} |
| 2276 | E_{1b}E₂E₃E₄ | AlkLEᵥEᵥᵢ | E₄*E₅ | E_{f} |
| 2277 | E₁ₐE₂EᵢE₄ | AlkLEᵥEᵥᵢ | E₄*E₅ | E_{f} |
| 2278 | E_{1b}E₂EᵢE₄ | AlkLEᵥEᵥᵢ | E₄*E₅ | E_{f} |
| 2279 | E₁ₐE₂EᵢᵢE₄ | AlkLEᵥEᵥᵢ | E₄*E₅ | E_{f} |
| 2280 | E_{1b}E₂EᵢᵢE₄ | AlkLEᵥEᵥᵢ | E₄*E₅ | E_{f} |
| 2281 | E₁ₐE₂EᵢᵢE_{iib}E₄ | AlkLEᵥEᵥᵢ | E₄*E₅ | E_{f} |
| 2282 | E_{1b}E₂EᵢᵢE_{iib}E₄ | AlkLEᵥEᵥᵢ | E₄*E₅ | E_{f} |
| 2283 | E₁ₐE₂E₃EᵢE₄ | AlkLEᵥEᵥᵢ | E₄*E₅ | E_{f} |
| 2284 | E_{1b}E₂E₃EᵢE₄ | AlkLEᵥEᵥᵢ | E₄*E₅ | E_{f} |
| 2285 | E₁ₐE₂E₃EᵢᵢE₄ | AlkLEᵥEᵥᵢ | E₄*E₅ | E_{f} |
| 2286 | E_{1b}E₂E₃EᵢᵢE₄ | AlkLEᵥEᵥᵢ | E₄*E₅ | E_{f} |
| 2287 | E₁ₐE₂E₃EᵢᵢE_{iib}E₄ | AlkLEᵥEᵥᵢ | E₄*E₅ | E_{f} |
| 2288 | E_{1b}E₂E₃EᵢᵢE_{iib}E₄ | AlkLEᵥEᵥᵢ | E₄*E₅ | E_{f} |
| 2289 | E₁ₐE₂Eᵢᵢᵢ | AlkLEᵥEᵥᵢ | E₄*E₅ | E_{f} |
| 2290 | E_{1b}E₂Eᵢᵢᵢ | AlkLEᵥEᵥᵢ | E₄*E₅ | E_{f} |
| 2291 | E₁ₐE₂E₃Eᵢᵢᵢ | AlkLEᵥEᵥᵢ | E₄*E₅ | E_{f} |
| 2292 | E_{1b}E₂E₃Eᵢᵢᵢ | AlkLEᵥEᵥᵢ | E₄*E₅ | E_{f} |
| 2293 | E₁ₐE₂EᵢᵢᵢE₄ | AlkLEᵥEᵥᵢ | E₄*E₅ | E_{f} |
| 2294 | E_{1b}E₂EᵢᵢᵢE₄ | AlkLEᵥEᵥᵢ | E₄*E₅ | E_{f} |
| 2295 | E₁ₐE₂E₃EᵢᵢᵢE₄ | AlkLEᵥEᵥᵢ | E₄*E₅ | E_{f} |
| 2296 | E_{1b}E₂E₃EᵢᵢᵢE₄ | AlkLEᵥEᵥᵢ | E₄*E₅ | E_{f} |
| 2297 | E₁ₐE₂Eᵢᵥ | AlkLEᵥEᵥᵢ | E₄*E₅ | E_{f} |
| 2298 | E_{1b}E₂Eᵢᵥ | AlkLEᵥEᵥᵢ | E₄*E₅ | E_{f} |
| 2299 | E₁ₐE₂E₃Eᵢᵥ | AlkLEᵥEᵥᵢ | E₄*E₅ | E_{f} |
| 2300 | E_{1b}E₂E₃Eᵢᵥ | AlkLEᵥEᵥᵢ | E₄*E₅ | E_{f} |
| 2301 | E₁ₐE₂EᵢᵥE₄ | AlkLEᵥEᵥᵢ | E₄*E₅ | E_{f} |
| 2302 | E_{1b}E₂EᵢᵥE₄ | AlkLEᵥEᵥᵢ | E₄*E₅ | E_{f} |
| 2303 | E₁ₐE₂E₃EᵢᵥE₄ | AlkLEᵥEᵥᵢ | E₄*E₅ | E_{f} |
| 2304 | E_{1b}E₂E₃EᵢᵥE₄ | AlkLEᵥEᵥᵢ | E₄*E₅ | E_{f} |
| 2305 | E₁ₐE₂ | Eᵥᵢᵢ | | |
| 2306 | E_{1b}E₂ | Eᵥᵢᵢ | | |
| 2307 | E₁ₐE₂E₃ | Eᵥᵢᵢ | | |
| 2308 | E_{1b}E₂E₃ | Eᵥᵢᵢ | | |
| 2309 | E₁ₐE₂Eᵢ | Eᵥᵢᵢ | | |
| 2310 | E_{1b}E₂Eᵢ | Eᵥᵢᵢ | | |
| 2311 | E₁ₐE₂Eᵢᵢ | Eᵥᵢᵢ | | |
| 2312 | E_{1b}E₂Eᵢᵢ | Eᵥᵢᵢ | | |
| 2313 | E₁ₐE₂EᵢᵢE_{iib} | Eᵥᵢᵢ | | |
| 2314 | E_{1b}E₂EᵢᵢE_{iib} | Eᵥᵢᵢ | | |
| 2315 | E₁ₐE₂E₃Eᵢ | Eᵥᵢᵢ | | |
| 2316 | E_{1b}E₂E₃Eᵢ | Eᵥᵢᵢ | | |
| 2317 | E₁ₐE₂E₃Eᵢᵢ | Eᵥᵢᵢ | | |
| 2318 | E_{1b}E₂E₃Eᵢᵢ | Eᵥᵢᵢ | | |
| 2319 | E₁ₐE₂E₃EᵢᵢE_{iib} | Eᵥᵢᵢ | | |
| 2320 | E_{1b}E₂E₃EᵢᵢE_{iib} | Eᵥᵢᵢ | | |
| 2321 | E₁ₐE₂E₄ | Eᵥᵢᵢ | | |
| 2322 | E_{1b}E₂E₄ | Eᵥᵢᵢ | | |
| 2323 | E₁ₐE₂E₃E₄ | Eᵥᵢᵢ | | |
| 2324 | E_{1b}E₂E₃E₄ | Eᵥᵢᵢ | | |
| 2325 | E₁ₐE₂EᵢE₄ | Eᵥᵢᵢ | | |
| 2326 | E_{1b}E₂EᵢE₄ | Eᵥᵢᵢ | | |
| 2327 | E₁ₐE₂EᵢᵢE₄ | Eᵥᵢᵢ | | |
| 2328 | E_{1b}E₂EᵢᵢE₄ | Eᵥᵢᵢ | | |
| 2329 | E₁ₐE₂EᵢᵢE_{iib}E₄ | Eᵥᵢᵢ | | |
| 2330 | E_{1b}E₂EᵢᵢE_{iib}E₄ | Eᵥᵢᵢ | | |
| 2331 | E₁ₐE₂E₃EᵢE₄ | Eᵥᵢᵢ | | |
| 2332 | E_{1b}E₂E₃EᵢE₄ | Eᵥᵢᵢ | | |
| 2333 | E₁ₐE₂E₃EᵢᵢE₄ | Eᵥᵢᵢ | | |
| 2334 | E_{1b}E₂E₃EᵢᵢE₄ | Eᵥᵢᵢ | | |
| 2335 | E₁ₐE₂E₃EᵢᵢE_{iib}E₄ | Eᵥᵢᵢ | | |
| 2336 | E_{1b}E₂E₃EᵢᵢE_{iib}E₄ | Eᵥᵢᵢ | | |
| 2337 | E₁ₐE₂Eᵢᵢᵢ | Eᵥᵢᵢ | | |
| 2338 | E_{1b}E₂Eᵢᵢᵢ | Eᵥᵢᵢ | | |
| 2339 | E₁ₐE₂E₃Eᵢᵢᵢ | Eᵥᵢᵢ | | |
| 2340 | E_{1b}E₂E₃Eᵢᵢᵢ | Eᵥᵢᵢ | | |
| 2341 | E₁ₐE₂EᵢᵢᵢE₄ | Eᵥᵢᵢ | | |
| 2342 | E_{1b}E₂EᵢᵢᵢE₄ | Eᵥᵢᵢ | | |
| 2343 | E₁ₐE₂E₃EᵢᵢᵢE₄ | Eᵥᵢᵢ | | |
| 2344 | E_{1b}E₂E₃EᵢᵢᵢE₄ | Eᵥᵢᵢ | | |
| 2345 | E₁ₐE₂Eᵢᵥ | Eᵥᵢᵢ | | |
| 2346 | E_{1b}E₂Eᵢᵥ | Eᵥᵢᵢ | | |
| 2347 | E₁ₐE₂E₃Eᵢᵥ | Eᵥᵢᵢ | | |
| 2348 | E_{1b}E₂E₃Eᵢᵥ | Eᵥᵢᵢ | | |
| 2349 | E₁ₐE₂EᵢᵥE₄ | Eᵥᵢᵢ | | |
| 2350 | E_{1b}E₂EᵢᵥE₄ | Eᵥᵢᵢ | | |
| 2351 | E₁ₐE₂E₃EᵢᵥE₄ | Eᵥᵢᵢ | | |
| 2352 | E_{1b}E₂E₃EᵢᵥE₄ | Eᵥᵢᵢ | | |
| 2353 | E₁ₐE₂ | Eᵥᵢᵢ | E₄* | |
| 2354 | E_{1b}E₂ | Eᵥᵢᵢ | E₄* | |
| 2355 | E₁ₐE₂E₃ | Eᵥᵢᵢ | E₄* | |
| 2356 | E_{1b}E₂E₃ | Eᵥᵢᵢ | E₄* | |
| 2357 | E₁ₐE₂Eᵢ | Eᵥᵢᵢ | E₄* | |
| 2358 | E_{1b}E₂Eᵢ | Eᵥᵢᵢ | E₄* | |
| 2359 | E₁ₐE₂Eᵢᵢ | Eᵥᵢᵢ | E₄* | |
| 2360 | E_{1b}E₂Eᵢᵢ | Eᵥᵢᵢ | E₄* | |
| 2361 | E₁ₐE₂EᵢᵢE_{iib} | Eᵥᵢᵢ | E₄* | |
| 2362 | E_{1b}E₂EᵢᵢE_{iib} | Eᵥᵢᵢ | E₄* | |
| 2363 | E₁ₐE₂E₃Eᵢ | Eᵥᵢᵢ | E₄* | |
| 2364 | E_{1b}E₂E₃Eᵢ | Eᵥᵢᵢ | E₄* | |
| 2365 | E₁ₐE₂E₃Eᵢᵢ | Eᵥᵢᵢ | E₄* | |
| 2366 | E_{1b}E₂E₃Eᵢᵢ | Eᵥᵢᵢ | E₄* | |
| 2367 | E₁ₐE₂E₃EᵢᵢE_{iib} | Eᵥᵢᵢ | E₄* | |
| 2368 | E_{1b}E₂E₃EᵢᵢE_{iib} | Eᵥᵢᵢ | E₄* | |
| 2369 | E₁ₐE₂E₄ | Eᵥᵢᵢ | E₄* | |
| 2370 | E_{1b}E₂E₄ | Eᵥᵢᵢ | E₄* | |
| 2371 | E₁ₐE₂E₃E₄ | Eᵥᵢᵢ | E₄* | |
| 2372 | E_{1b}E₂E₃E₄ | Eᵥᵢᵢ | E₄* | |
| 2373 | E₁ₐE₂EᵢE₄ | Eᵥᵢᵢ | E₄* | |
| 2374 | E_{1b}E₂EᵢE₄ | Eᵥᵢᵢ | E₄* | |
| 2375 | E₁ₐE₂EᵢᵢE₄ | Eᵥᵢᵢ | E₄* | |
| 2376 | E_{1b}E₂EᵢᵢE₄ | Eᵥᵢᵢ | E₄* | |
| 2377 | E₁ₐE₂EᵢᵢE_{iib}E₄ | Eᵥᵢᵢ | E₄* | |
| 2378 | E_{1b}E₂EᵢᵢE_{iib}E₄ | Eᵥᵢᵢ | E₄* | |
| 2379 | E₁ₐE₂E₃EᵢE₄ | Eᵥᵢᵢ | E₄* | |
| 2380 | E_{1b}E₂E₃EᵢE₄ | Eᵥᵢᵢ | E₄* | |
| 2381 | E₁ₐE₂E₃EᵢᵢE₄ | Eᵥᵢᵢ | E₄* | |
| 2382 | E_{1b}E₂E₃EᵢᵢE₄ | Eᵥᵢᵢ | E₄* | |
| 2383 | E₁ₐE₂E₃EᵢᵢE_{iib}E₄ | Eᵥᵢᵢ | E₄* | |
| 2384 | E_{1b}E₂E₃EᵢᵢE_{iib}E₄ | Eᵥᵢᵢ | E₄* | |
| 2385 | E₁ₐE₂Eᵢᵢᵢ | Eᵥᵢᵢ | E₄* | |
| 2386 | E_{1b}E₂Eᵢᵢᵢ | Eᵥᵢᵢ | E₄* | |
| 2387 | E₁ₐE₂E₃Eᵢᵢᵢ | Eᵥᵢᵢ | E₄* | |
| 2388 | E_{1b}E₂E₃Eᵢᵢᵢ | Eᵥᵢᵢ | E₄* | |
| 2389 | E₁ₐE₂EᵢᵢᵢE₄ | Eᵥᵢᵢ | E₄* | |
| 2390 | E_{1b}E₂EᵢᵢᵢE₄ | Eᵥᵢᵢ | E₄* | |
| 2391 | E₁ₐE₂E₃EᵢᵢᵢE₄ | Eᵥᵢᵢ | E₄* | |
| 2392 | E_{1b}E₂E₃EᵢᵢᵢE₄ | Eᵥᵢᵢ | E₄* | |
| 2393 | E₁ₐE₂Eᵢᵥ | Eᵥᵢᵢ | E₄* | |
| 2394 | E_{1b}E₂Eᵢᵥ | Eᵥᵢᵢ | E₄* | |
| 2395 | E₁ₐE₂E₃Eᵢᵥ | Eᵥᵢᵢ | E₄* | |
| 2396 | E_{1b}E₂E₃Eᵢᵥ | Eᵥᵢᵢ | E₄* | |
| 2397 | E₁ₐE₂EᵢᵥE₄ | Eᵥᵢᵢ | E₄* | |
| 2398 | E_{1b}E₂EᵢᵥE₄ | Eᵥᵢᵢ | E₄* | |
| 2399 | E₁ₐE₂E₃EᵢᵥE₄ | Eᵥᵢᵢ | E₄* | |
| 2400 | E_{1b}E₂E₃EᵢᵥE₄ | Eᵥᵢᵢ | E₄* | |
| 2401 | E₁ₐE₂ | Eᵥᵢᵢ | E₅ | |
| 2402 | E_{1b}E₂ | Eᵥᵢᵢ | E₅ | |
| 2403 | E₁ₐE₂E₃ | Eᵥᵢᵢ | E₅ | |
| 2404 | E_{1b}E₂E₃ | Eᵥᵢᵢ | E₅ | |
| 2405 | E₁ₐE₂Eᵢ | Eᵥᵢᵢ | E₅ | |
| 2406 | E_{1b}E₂Eᵢ | Eᵥᵢᵢ | E₅ | |
| 2407 | E₁ₐE₂Eᵢᵢ | Eᵥᵢᵢ | E₅ | |
| 2408 | E_{1b}E₂Eᵢᵢ | Eᵥᵢᵢ | E₅ | |
| 2409 | E₁ₐE₂EᵢᵢE_{iib} | Eᵥᵢᵢ | E₅ | |
| 2410 | E_{1b}E₂EᵢᵢE_{iib} | Eᵥᵢᵢ | E₅ | |
| 2411 | E₁ₐE₂E₃Eᵢ | Eᵥᵢᵢ | E₅ | |
| 2412 | E_{1b}E₂E₃Eᵢ | Eᵥᵢᵢ | E₅ | |
| 2413 | E₁ₐE₂E₃Eᵢᵢ | Eᵥᵢᵢ | E₅ | |
| 2414 | E_{1b}E₂E₃Eᵢᵢ | Eᵥᵢᵢ | E₅ | |
| 2415 | E₁ₐE₂E₃EᵢᵢE_{iib} | Eᵥᵢᵢ | E₅ | |
| 2416 | E_{1b}E₂E₃EᵢᵢE_{iib} | Eᵥᵢᵢ | E₅ | |
| 2417 | E₁ₐE₂E₄ | Eᵥᵢᵢ | E₅ | |
| 2418 | E_{1b}E₂E₄ | Eᵥᵢᵢ | E₅ | |
| 2419 | E₁ₐE₂E₃E₄ | Eᵥᵢᵢ | E₅ | |
| 2420 | E_{1b}E₂E₃E₄ | Eᵥᵢᵢ | E₅ | |
| 2421 | E₁ₐE₂EᵢE₄ | Eᵥᵢᵢ | E₅ | |
| 2422 | E_{1b}E₂EᵢE₄ | Eᵥᵢᵢ | E₅ | |
| 2423 | E₁ₐE₂EᵢᵢE₄ | Eᵥᵢᵢ | E₅ | |
| 2424 | E_{1b}E₂EᵢᵢE₄ | Eᵥᵢᵢ | E₅ | |
| 2425 | E₁ₐE₂EᵢᵢE_{iib}E₄ | Eᵥᵢᵢ | E₅ | |
| 2426 | E_{1b}E₂EᵢᵢE_{iib}E₄ | Eᵥᵢᵢ | E₅ | |
| 2427 | E₁ₐE₂E₃EᵢE₄ | Eᵥᵢᵢ | E₅ | |
| 2428 | E_{1b}E₂E₃EᵢE₄ | Eᵥᵢᵢ | E₅ | |
| 2429 | E₁ₐE₂E₃EᵢᵢE₄ | Eᵥᵢᵢ | E₅ | |
| 2430 | E_{1b}E₂E₃EᵢᵢE₄ | Eᵥᵢᵢ | E₅ | |
| 2431 | E₁ₐE₂E₃EᵢᵢE_{iib}E₄ | Eᵥᵢᵢ | E₅ | |
| 2432 | E_{1b}E₂E₃EᵢᵢE_{iib}E₄ | Eᵥᵢᵢ | E₅ | |
| 2433 | E₁ₐE₂Eᵢᵢᵢ | Eᵥᵢᵢ | E₅ | |
| 2434 | E_{1b}E₂Eᵢᵢᵢ | Eᵥᵢᵢ | E₅ | |
| 2435 | E₁ₐE₂E₃Eᵢᵢᵢ | Eᵥᵢᵢ | E₅ | |
| 2436 | E_{1b}E₂E₃Eᵢᵢᵢ | Eᵥᵢᵢ | E₅ | |
| 2437 | E₁ₐE₂EᵢᵢᵢE₄ | Eᵥᵢᵢ | E₅ | |
| 2438 | E_{1b}E₂EᵢᵢᵢE₄ | Eᵥᵢᵢ | E₅ | |
| 2439 | E₁ₐE₂E₃EᵢᵢᵢE₄ | Eᵥᵢᵢ | E₅ | |
| 2440 | E_{1b}E₂E₃EᵢᵢᵢE₄ | Eᵥᵢᵢ | E₅ | |
| 2441 | E₁ₐE₂Eᵢᵥ | Eᵥᵢᵢ | E₅ | |
| 2442 | E_{1b}E₂Eᵢᵥ | Eᵥᵢᵢ | E₅ | |
| 2443 | E₁ₐE₂E₃Eᵢᵥ | Eᵥᵢᵢ | E₅ | |
| 2444 | E_{1b}E₂E₃Eᵢᵥ | Eᵥᵢᵢ | E₅ | |
| 2445 | E₁ₐE₂EᵢᵥE₄ | Eᵥᵢᵢ | E₅ | |
| 2446 | E_{1b}E₂EᵢᵥE₄ | Eᵥᵢᵢ | E₅ | |
| 2447 | E₁ₐE₂E₃EᵢᵥE₄ | Eᵥᵢᵢ | E₅ | |
| 2448 | E_{1b}E₂E₃EᵢᵥE₄ | Eᵥᵢᵢ | E₅ | |
| 2449 | E₁ₐE₂ | Eᵥᵢᵢ | E₄*E₅ | |
| 2450 | E_{1b}E₂ | Eᵥᵢᵢ | E₄*E₅ | |
| 2451 | E₁ₐE₂E₃ | Eᵥᵢᵢ | E₄*E₅ | |
| 2452 | E_{1b}E₂E₃ | Eᵥᵢᵢ | E₄*E₅ | |
| 2453 | E₁ₐE₂Eᵢ | Eᵥᵢᵢ | E₄*E₅ | |
| 2454 | E_{1b}E₂Eᵢ | Eᵥᵢᵢ | E₄*E₅ | |
| 2455 | E₁ₐE₂Eᵢᵢ | Eᵥᵢᵢ | E₄*E₅ | |
| 2456 | E_{1b}E₂Eᵢᵢ | Eᵥᵢᵢ | E₄*E₅ | |
| 2457 | E₁ₐE₂EᵢᵢE_{iib} | Eᵥᵢᵢ | E₄*E₅ | |
| 2458 | E_{1b}E₂EᵢᵢE_{iib} | Eᵥᵢᵢ | E₄*E₅ | |
| 2459 | E₁ₐE₂E₃Eᵢ | Eᵥᵢᵢ | E₄*E₅ | |
| 2460 | E_{1b}E₂E₃Eᵢ | Eᵥᵢᵢ | E₄*E₅ | |
| 2461 | E₁ₐE₂E₃Eᵢᵢ | Eᵥᵢᵢ | E₄*E₅ | |
| 2462 | E_{1b}E₂E₃Eᵢᵢ | Eᵥᵢᵢ | E₄*E₅ | |
| 2463 | E₁ₐE₂E₃EᵢᵢE_{iib} | Eᵥᵢᵢ | E₄*E₅ | |
| 2464 | E_{1b}E₂E₃EᵢᵢE_{iib} | Eᵥᵢᵢ | E₄*E₅ | |
| 2465 | E₁ₐE₂E₄ | Eᵥᵢᵢ | E₄*E₅ | |
| 2466 | E_{1b}E₂E₄ | Eᵥᵢᵢ | E₄*E₅ | |
| 2467 | E₁ₐE₂E₃E₄ | Eᵥᵢᵢ | E₄*E₅ | |
| 2468 | E_{1b}E₂E₃E₄ | Eᵥᵢᵢ | E₄*E₅ | |
| 2469 | E₁ₐE₂EᵢE₄ | Eᵥᵢᵢ | E₄*E₅ | |
| 2470 | E_{1b}E₂EᵢE₄ | Eᵥᵢᵢ | E₄*E₅ | |
| 2471 | E₁ₐE₂EᵢᵢE₄ | Eᵥᵢᵢ | E₄*E₅ | |
| 2472 | E_{1b}E₂EᵢᵢE₄ | Eᵥᵢᵢ | E₄*E₅ | |
| 2473 | E₁ₐE₂EᵢᵢE_{iib}E₄ | Eᵥᵢᵢ | E₄*E₅ | |
| 2474 | E_{1b}E₂EᵢᵢE_{iib}E₄ | Eᵥᵢᵢ | E₄*E₅ | |
| 2475 | E₁ₐE₂E₃EᵢE₄ | Eᵥᵢᵢ | E₄*E₅ | |
| 2476 | E_{1b}E₂E₃EᵢE₄ | Eᵥᵢᵢ | E₄*E₅ | |
| 2477 | E₁ₐE₂E₃EᵢᵢE₄ | Eᵥᵢᵢ | E₄*E₅ | |
| 2478 | E_{1b}E₂E₃EᵢᵢE₄ | Eᵥᵢᵢ | E₄*E₅ | |
| 2479 | E₁ₐE₂E₃EᵢᵢE_{iib}E₄ | Eᵥᵢᵢ | E₄*E₅ | |
| 2480 | E_{1b}E₂E₃EᵢᵢE_{iib}E₄ | Eᵥᵢᵢ | E₄*E₅ | |
| 2481 | E₁ₐE₂Eᵢᵢᵢ | Eᵥᵢᵢ | E₄*E₅ | |
| 2482 | E_{1b}E₂Eᵢᵢᵢ | Eᵥᵢᵢ | E₄*E₅ | |
| 2483 | E₁ₐE₂E₃Eᵢᵢᵢ | Eᵥᵢᵢ | E₄*E₅ | |
| 2484 | E_{1b}E₂E₃Eᵢᵢᵢ | Eᵥᵢᵢ | E₄*E₅ | |
| 2485 | E₁ₐE₂EᵢᵢᵢE₄ | Eᵥᵢᵢ | E₄*E₅ | |
| 2486 | E_{1b}E₂EᵢᵢᵢE₄ | Eᵥᵢᵢ | E₄*E₅ | |
| 2487 | E₁ₐE₂E₃EᵢᵢᵢE₄ | Eᵥᵢᵢ | E₄*E₅ | |
| 2488 | E_{1b}E₂E₃EᵢᵢᵢE₄ | Eᵥᵢᵢ | E₄*E₅ | |
| 2489 | E₁ₐE₂Eᵢᵥ | Eᵥᵢᵢ | E₄*E₅ | |
| 2490 | E_{1b}E₂Eᵢᵥ | Eᵥᵢᵢ | E₄*E₅ | |
| 2491 | E₁ₐE₂E₃Eᵢᵥ | Eᵥᵢᵢ | E₄*E₅ | |
| 2492 | E_{1b}E₂E₃Eᵢᵥ | Eᵥᵢᵢ | E₄*E₅ | |
| 2493 | E₁ₐE₂EᵢᵥE₄ | Eᵥᵢᵢ | E₄*E₅ | |
| 2494 | E_{1b}E₂EᵢᵥE₄ | Eᵥᵢᵢ | E₄*E₅ | |
| 2495 | E₁ₐE₂E₃EᵢᵥE₄ | Eᵥᵢᵢ | E₄*E₅ | |
| 2496 | E_{1b}E₂E₃EᵢᵥE₄ | Eᵥᵢᵢ | E₄*E₅ | |
| 2497 | E₁ₐE₂ | AlkLEᵥᵢᵢ | | |
| 2498 | E_{1b}E₂ | AlkLEᵥᵢᵢ | | |
| 2499 | E₁ₐE₂E₃ | AlkLEᵥᵢᵢ | | |
| 2500 | E_{1b}E₂E₃ | AlkLEᵥᵢᵢ | | |
| 2501 | E₁ₐE₂Eᵢ | AlkLEᵥᵢᵢ | | |
| 2502 | E_{1b}E₂Eᵢ | AlkLEᵥᵢᵢ | | |
| 2503 | E₁ₐE₂Eᵢᵢ | AlkLEᵥᵢᵢ | | |
| 2504 | E_{1b}E₂Eᵢᵢ | AlkLEᵥᵢᵢ | | |
| 2505 | E₁ₐE₂EᵢᵢE_{iib} | AlkLEᵥᵢᵢ | | |
| 2506 | E_{1b}E₂EᵢᵢE_{iib} | AlkLEᵥᵢᵢ | | |
| 2507 | E₁ₐE₂E₃Eᵢ | AlkLEᵥᵢᵢ | | |
| 2508 | E_{1b}E₂E₃Eᵢ | AlkLEᵥᵢᵢ | | |
| 2509 | E₁ₐE₂E₃Eᵢᵢ | AlkLEᵥᵢᵢ | | |
| 2510 | E_{1b}E₂E₃Eᵢᵢ | AlkLEᵥᵢᵢ | | |
| 2511 | E₁ₐE₂E₃EᵢᵢE_{iib} | AlkLEᵥᵢᵢ | | |
| 2512 | E_{1b}E₂E₃EᵢᵢE_{iib} | AlkLEᵥᵢᵢ | | |
| 2513 | E₁ₐE₂E₄ | AlkLEᵥᵢᵢ | | |
| 2514 | E_{1b}E₂E₄ | AlkLEᵥᵢᵢ | | |
| 2515 | E₁ₐE₂E₃E₄ | AlkLEᵥᵢᵢ | | |
| 2516 | E_{1b}E₂E₃E₄ | AlkLEᵥᵢᵢ | | |
| 2517 | E₁ₐE₂EᵢE₄ | AlkLEᵥᵢᵢ | | |
| 2518 | E_{1b}E₂EᵢE₄ | AlkLEᵥᵢᵢ | | |
| 2519 | E₁ₐE₂EᵢᵢE₄ | AlkLEᵥᵢᵢ | | |
| 2520 | E_{1b}E₂EᵢᵢE₄ | AlkLEᵥᵢᵢ | | |
| 2521 | E₁ₐE₂EᵢᵢE_{iib}E₄ | AlkLEᵥᵢᵢ | | |
| 2522 | E_{1b}E₂EᵢᵢE_{iib}E₄ | AlkLEᵥᵢᵢ | | |
| 2523 | E₁ₐE₂E₃EᵢE₄ | AlkLEᵥᵢᵢ | | |
| 2524 | E_{1b}E₂E₃EᵢE₄ | AlkLEᵥᵢᵢ | | |
| 2525 | E₁ₐE₂E₃EᵢᵢE₄ | AlkLEᵥᵢᵢ | | |
| 2526 | E_{1b}E₂E₃EᵢᵢE₄ | AlkLEᵥᵢᵢ | | |
| 2527 | E₁ₐE₂E₃EᵢᵢE_{iib}E₄ | AlkLEᵥᵢᵢ | | |
| 2528 | E_{1b}E₂E₃EᵢᵢE_{iib}E₄ | AlkLEᵥᵢᵢ | | |
| 2529 | E₁ₐE₂Eᵢᵢᵢ | AlkLEᵥᵢᵢ | | |
| 2530 | E_{1b}E₂Eᵢᵢᵢ | AlkLEᵥᵢᵢ | | |
| 2531 | E₁ₐE₂E₃Eᵢᵢᵢ | AlkLEᵥᵢᵢ | | |
| 2532 | E_{1b}E₂E₃Eᵢᵢᵢ | AlkLEᵥᵢᵢ | | |
| 2533 | E₁ₐE₂EᵢᵢᵢE₄ | AlkLEᵥᵢᵢ | | |
| 2534 | E_{1b}E₂EᵢᵢᵢE₄ | AlkLEᵥᵢᵢ | | |
| 2535 | E₁ₐE₂E₃EᵢᵢᵢE₄ | AlkLEᵥᵢᵢ | | |
| 2536 | E_{1b}E₂E₃EᵢᵢᵢE₄ | AlkLEᵥᵢᵢ | | |
| 2537 | E₁ₐE₂Eᵢᵥ | AlkLEᵥᵢᵢ | | |
| 2538 | E_{1b}E₂Eᵢᵥ | AlkLEᵥᵢᵢ | | |
| 2539 | E₁ₐE₂E₃Eᵢᵥ | AlkLEᵥᵢᵢ | | |
| 2540 | E_{1b}E₂E₃Eᵢᵥ | AlkLEᵥᵢᵢ | | |
| 2541 | E₁ₐE₂EᵢᵥE₄ | AlkLEᵥᵢᵢ | | |
| 2542 | E_{1b}E₂EᵢᵥE₄ | AlkLEᵥᵢᵢ | | |
| 2543 | E₁ₐE₂E₃EᵢᵥE₄ | AlkLEᵥᵢᵢ | | |
| 2544 | E_{1b}E₂E₃EᵢᵥE₄ | AlkLEᵥᵢᵢ | | |
| 2545 | E₁ₐE₂ | AlkLEᵥᵢᵢ | E₄* | |
| 2546 | E_{1b}E₂ | AlkLEᵥᵢᵢ | E₄* | |
| 2547 | E₁ₐE₂E₃ | AlkLEᵥᵢᵢ | E₄* | |
| 2548 | E_{1b}E₂E₃ | AlkLEᵥᵢᵢ | E₄* | |
| 2549 | E₁ₐE₂Eᵢ | AlkLEᵥᵢᵢ | E₄* | |
| 2550 | E_{1b}E₂Eᵢ | AlkLEᵥᵢᵢ | E₄* | |
| 2551 | E₁ₐE₂Eᵢᵢ | AlkLEᵥᵢᵢ | E₄* | |
| 2552 | E_{1b}E₂Eᵢᵢ | AlkLEᵥᵢᵢ | E₄* | |
| 2553 | E₁ₐE₂EᵢᵢE_{iib} | AlkLEᵥᵢᵢ | E₄* | |
| 2554 | E_{1b}E₂EᵢᵢE_{iib} | AlkLEᵥᵢᵢ | E₄* | |
| 2555 | E₁ₐE₂E₃Eᵢ | AlkLEᵥᵢᵢ | E₄* | |
| 2556 | E_{1b}E₂E₃Eᵢ | AlkLEᵥᵢᵢ | E₄* | |
| 2557 | E₁ₐE₂E₃Eᵢᵢ | AlkLEᵥᵢᵢ | E₄* | |
| 2558 | E_{1b}E₂E₃Eᵢᵢ | AlkLEᵥᵢᵢ | E₄* | |
| 2559 | E₁ₐE₂E₃EᵢᵢE_{iib} | AlkLEᵥᵢᵢ | E₄* | |
| 2560 | E_{1b}E₂E₃EᵢᵢE_{iib} | AlkLEᵥᵢᵢ | E₄* | |
| 2561 | E₁ₐE₂E₄ | AlkLEᵥᵢᵢ | E₄* | |
| 2562 | E_{1b}E₂E₄ | AlkLEᵥᵢᵢ | E₄* | |
| 2563 | E₁ₐE₂E₃E₄ | AlkLEᵥᵢᵢ | E₄* | |
| 2564 | E_{1b}E₂E₃E₄ | AlkLEᵥᵢᵢ | E₄* | |
| 2565 | E₁ₐE₂EᵢE₄ | AlkLEᵥᵢᵢ | E₄* | |
| 2566 | E_{1b}E₂EᵢE₄ | AlkLEᵥᵢᵢ | E₄* | |
| 2567 | E₁ₐE₂EᵢᵢE₄ | AlkLEᵥᵢᵢ | E₄* | |
| 2568 | E_{1b}E₂EᵢᵢE₄ | AlkLEᵥᵢᵢ | E₄* | |
| 2569 | E₁ₐE₂EᵢᵢE_{iib}E₄ | AlkLEᵥᵢᵢ | E₄* | |
| 2570 | E_{1b}E₂EᵢᵢE_{iib}E₄ | AlkLEᵥᵢᵢ | E₄* | |
| 2571 | E₁ₐE₂E₃EᵢE₄ | AlkLEᵥᵢᵢ | E₄* | |
| 2572 | E_{1b}E₂E₃EᵢE₄ | AlkLEᵥᵢᵢ | E₄* | |
| 2573 | E₁ₐE₂E₃EᵢᵢE₄ | AlkLEᵥᵢᵢ | E₄* | |
| 2574 | E_{1b}E₂E₃EᵢᵢE₄ | AlkLEᵥᵢᵢ | E₄* | |
| 2575 | E₁ₐE₂E₃EᵢᵢE_{iib}E₄ | AlkLEᵥᵢᵢ | E₄* | |
| 2576 | E_{1b}E₂E₃EᵢᵢE_{iib}E₄ | AlkLEᵥᵢᵢ | E₄* | |
| 2577 | E₁ₐE₂Eᵢᵢᵢ | AlkLEᵥᵢᵢ | E₄* | |
| 2578 | E_{1b}E₂Eᵢᵢᵢ | AlkLEᵥᵢᵢ | E₄* | |
| 2579 | E₁ₐE₂E₃Eᵢᵢᵢ | AlkLEᵥᵢᵢ | E₄* | |
| 2580 | E_{1b}E₂E₃Eᵢᵢᵢ | AlkLEᵥᵢᵢ | E₄* | |
| 2581 | E₁ₐE₂EᵢᵢᵢE₄ | AlkLEᵥᵢᵢ | E₄* | |
| 2582 | E_{1b}E₂EᵢᵢᵢE₄ | AlkLEᵥᵢᵢ | E₄* | |
| 2583 | E₁ₐE₂E₃EᵢᵢᵢE₄ | AlkLEᵥᵢᵢ | E₄* | |
| 2584 | E_{1b}E₂E₃EᵢᵢᵢE₄ | AlkLEᵥᵢᵢ | E₄* | |
| 2585 | E₁ₐE₂Eᵢᵥ | AlkLEᵥᵢᵢ | E₄* | |
| 2586 | E_{1b}E₂Eᵢᵥ | AlkLEᵥᵢᵢ | E₄* | |
| 2587 | E₁ₐE₂E₃Eᵢᵥ | AlkLEᵥᵢᵢ | E₄* | |
| 2588 | E_{1b}E₂E₃Eᵢᵥ | AlkLEᵥᵢᵢ | E₄* | |
| 2589 | E₁ₐE₂EᵢᵥE₄ | AlkLEᵥᵢᵢ | E₄* | |
| 2590 | E_{1b}E₂EᵢᵥE₄ | AlkLEᵥᵢᵢ | E₄* | |
| 2591 | E₁ₐE₂E₃EᵢᵥE₄ | AlkLEᵥᵢᵢ | E₄* | |
| 2592 | E_{1b}E₂E₃EᵢᵥE₄ | AlkLEᵥᵢᵢ | E₄* | |
| 2593 | E₁ₐE₂ | AlkLEᵥᵢᵢ | E₅ | |
| 2594 | E_{1b}E₂ | AlkLEᵥᵢᵢ | E₅ | |
| 2595 | E₁ₐE₂E₃ | AlkLEᵥᵢᵢ | E₅ | |
| 2596 | E_{1b}E₂E₃ | AlkLEᵥᵢᵢ | E₅ | |
| 2597 | E₁ₐE₂Eᵢ | AlkLEᵥᵢᵢ | E₅ | |
| 2598 | E_{1b}E₂Eᵢ | AlkLEᵥᵢᵢ | E₅ | |
| 2599 | E₁ₐE₂Eᵢᵢ | AlkLEᵥᵢᵢ | E₅ | |
| 2600 | E_{1b}E₂Eᵢᵢ | AlkLEᵥᵢᵢ | E₅ | |
| 2601 | E₁ₐE₂EᵢᵢE_{iib} | AlkLEᵥᵢᵢ | E₅ | |
| 2602 | E_{1b}E₂EᵢᵢE_{iib} | AlkLEᵥᵢᵢ | E₅ | |
| 2603 | E₁ₐE₂E₃Eᵢ | AlkLEᵥᵢᵢ | E₅ | |
| 2604 | E_{1b}E₂E₃Eᵢ | AlkLEᵥᵢᵢ | E₅ | |
| 2605 | E₁ₐE₂E₃Eᵢᵢ | AlkLEᵥᵢᵢ | E₅ | |
| 2606 | E_{1b}E₂E₃Eᵢᵢ | AlkLEᵥᵢᵢ | E₅ | |
| 2607 | E₁ₐE₂E₃EᵢᵢE_{iib} | AlkLEᵥᵢᵢ | E₅ | |
| 2608 | E_{1b}E₂E₃EᵢᵢE_{iib} | AlkLEᵥᵢᵢ | E₅ | |
| 2609 | E₁ₐE₂E₄ | AlkLEᵥᵢᵢ | E₅ | |
| 2610 | E_{1b}E₂E₄ | AlkLEᵥᵢᵢ | E₅ | |
| 2611 | E₁ₐE₂E₃E₄ | AlkLEᵥᵢᵢ | E₅ | |
| 2612 | E_{1b}E₂E₃E₄ | AlkLEᵥᵢᵢ | E₅ | |
| 2613 | E₁ₐE₂EᵢE₄ | AlkLEᵥᵢᵢ | E₅ | |
| 2614 | E_{1b}E₂EᵢE₄ | AlkLEᵥᵢᵢ | E₅ | |
| 2615 | E₁ₐE₂EᵢᵢE₄ | AlkLEᵥᵢᵢ | E₅ | |
| 2616 | E_{1b}E₂EᵢᵢE₄ | AlkLEᵥᵢᵢ | E₅ | |
| 2617 | E₁ₐE₂EᵢᵢE_{iib}E₄ | AlkLEᵥᵢᵢ | E₅ | |
| 2618 | E_{1b}E₂EᵢᵢE_{iib}E₄ | AlkLEᵥᵢᵢ | E₅ | |
| 2619 | E₁ₐE₂E₃EᵢE₄ | AlkLEᵥᵢᵢ | E₅ | |
| 2620 | E_{1b}E₂E₃EᵢE₄ | AlkLEᵥᵢᵢ | E₅ | |
| 2621 | E₁ₐE₂E₃EᵢᵢE₄ | AlkLEᵥᵢᵢ | E₅ | |
| 2622 | E_{1b}E₂E₃EᵢᵢE₄ | AlkLEᵥᵢᵢ | E₅ | |
| 2623 | E₁ₐE₂E₃EᵢᵢE_{iib}E₄ | AlkLEᵥᵢᵢ | E₅ | |
| 2624 | E_{1b}E₂E₃EᵢᵢE_{iib}E₄ | AlkLEᵥᵢᵢ | E₅ | |
| 2625 | E₁ₐE₂Eᵢᵢᵢ | AlkLEᵥᵢᵢ | E₅ | |
| 2626 | E_{1b}E₂Eᵢᵢᵢ | AlkLEᵥᵢᵢ | E₅ | |
| 2627 | E₁ₐE₂E₃Eᵢᵢᵢ | AlkLEᵥᵢᵢ | E₅ | |
| 2628 | E_{1b}E₂E₃Eᵢᵢᵢ | AlkLEᵥᵢᵢ | E₅ | |
| 2629 | E₁ₐE₂EᵢᵢᵢE₄ | AlkLEᵥᵢᵢ | E₅ | |
| 2630 | E_{1b}E₂EᵢᵢᵢE₄ | AlkLEᵥᵢᵢ | E₅ | |
| 2631 | E₁ₐE₂E₃EᵢᵢᵢE₄ | AlkLEᵥᵢᵢ | E₅ | |
| 2632 | E_{1b}E₂E₃EᵢᵢᵢE₄ | AlkLEᵥᵢᵢ | E₅ | |
| 2633 | E₁ₐE₂Eᵢᵥ | AlkLEᵥᵢᵢ | E₅ | |
| 2634 | E_{1b}E₂Eᵢᵥ | AlkLEᵥᵢᵢ | E₅ | |
| 2635 | E₁ₐE₂E₃Eᵢᵥ | AlkLEᵥᵢᵢ | E₅ | |
| 2636 | E_{1b}E₂E₃Eᵢᵥ | AlkLEᵥᵢᵢ | E₅ | |
| 2637 | E₁ₐE₂EᵢᵥE₄ | AlkLEᵥᵢᵢ | E₅ | |
| 2638 | E_{1b}E₂EᵢᵥE₄ | AlkLEᵥᵢᵢ | E₅ | |
| 2639 | E₁ₐE₂E₃EᵢᵥE₄ | AlkLEᵥᵢᵢ | E₅ | |
| 2640 | E_{1b}E₂E₃EᵢᵥE₄ | AlkLEᵥᵢᵢ | E₅ | |
| 2641 | E₁ₐE₂ | AlkLEᵥᵢᵢ | E₄*E₅ | |
| 2642 | E_{1b}E₂ | AlkLEᵥᵢᵢ | E₄*E₅ | |
| 2643 | E₁ₐE₂E₃ | AlkLEᵥᵢᵢ | E₄*E₅ | |
| 2644 | E_{1b}E₂E₃ | AlkLEᵥᵢᵢ | E₄*E₅ | |
| 2645 | E₁ₐE₂Eᵢ | AlkLEᵥᵢᵢ | E₄*E₅ | |
| 2646 | E_{1b}E₂Eᵢ | AlkLEᵥᵢᵢ | E₄*E₅ | |
| 2647 | E₁ₐE₂Eᵢᵢ | AlkLEᵥᵢᵢ | E₄*E₅ | |
| 2648 | E_{1b}E₂Eᵢᵢ | AlkLEᵥᵢᵢ | E₄*E₅ | |
| 2649 | E₁ₐE₂EᵢᵢE_{iib} | AlkLEᵥᵢᵢ | E₄*E₅ | |
| 2650 | E_{1b}E₂EᵢᵢE_{iib} | AlkLEᵥᵢᵢ | E₄*E₅ | |
| 2651 | E₁ₐE₂E₃Eᵢ | AlkLEᵥᵢᵢ | E₄*E₅ | |
| 2652 | E_{1b}E₂E₃Eᵢ | AlkLEᵥᵢᵢ | E₄*E₅ | |
| 2653 | E₁ₐE₂E₃Eᵢᵢ | AlkLEᵥᵢᵢ | E₄*E₅ | |
| 2654 | E_{1b}E₂E₃Eᵢᵢ | AlkLEᵥᵢᵢ | E₄*E₅ | |
| 2655 | E₁ₐE₂E₃EᵢᵢE_{iib} | AlkLEᵥᵢᵢ | E₄*E₅ | |
| 2656 | E_{1b}E₂E₃EᵢᵢE_{iib} | AlkLEᵥᵢᵢ | E₄*E₅ | |
| 2657 | E₁ₐE₂E₄ | AlkLEᵥᵢᵢ | E₄*E₅ | |
| 2658 | E_{1b}E₂E₄ | AlkLEᵥᵢᵢ | E₄*E₅ | |
| 2659 | E₁ₐE₂E₃E₄ | AlkLEᵥᵢᵢ | E₄*E₅ | |
| 2660 | E_{1b}E₂E₃E₄ | AlkLEᵥᵢᵢ | E₄*E₅ | |
| 2661 | E₁ₐE₂EᵢE₄ | AlkLEᵥᵢᵢ | E₄*E₅ | |
| 2662 | E_{1b}E₂EᵢE₄ | AlkLEᵥᵢᵢ | E₄*E₅ | |
| 2663 | E₁ₐE₂EᵢᵢE₄ | AlkLEᵥᵢᵢ | E₄*E₅ | |
| 2664 | E_{1b}E₂EᵢᵢE₄ | AlkLEᵥᵢᵢ | E₄*E₅ | |
| 2665 | E₁ₐE₂EᵢᵢE_{iib}E₄ | AlkLEᵥᵢᵢ | E₄*E₅ | |
| 2666 | E_{1b}E₂EᵢᵢE_{iib}E₄ | AlkLEᵥᵢᵢ | E₄*E₅ | |
| 2667 | E₁ₐE₂E₃EᵢE₄ | AlkLEᵥᵢᵢ | E₄*E₅ | |
| 2668 | E_{1b}E₂E₃EᵢE₄ | AlkLEᵥᵢᵢ | E₄*E₅ | |
| 2669 | E₁ₐE₂E₃EᵢᵢE₄ | AlkLEᵥᵢᵢ | E₄*E₅ | |
| 2670 | E_{1b}E₂E₃EᵢᵢE₄ | AlkLEᵥᵢᵢ | E₄*E₅ | |
| 2671 | E₁ₐE₂E₃EᵢᵢE_{iib}E₄ | AlkLEᵥᵢᵢ | E₄*E₅ | |
| 2672 | E_{1b}E₂E₃EᵢᵢE_{iib}E₄ | AlkLEᵥᵢᵢ | E₄*E₅ | |
| 2673 | E₁ₐE₂Eᵢᵢᵢ | AlkLEᵥᵢᵢ | E₄*E₅ | |
| 2674 | E_{1b}E₂Eᵢᵢᵢ | AlkLEᵥᵢᵢ | E₄*E₅ | |
| 2675 | E₁ₐE₂E₃Eᵢᵢᵢ | AlkLEᵥᵢᵢ | E₄*E₅ | |
| 2676 | E_{1b}E₂E₃Eᵢᵢᵢ | AlkLEᵥᵢᵢ | E₄*E₅ | |
| 2677 | E₁ₐE₂EᵢᵢᵢE₄ | AlkLEᵥᵢᵢ | E₄*E₅ | |
| 2678 | E_{1b}E₂EᵢᵢᵢE₄ | AlkLEᵥᵢᵢ | E₄*E₅ | |
| 2679 | E₁ₐE₂E₃EᵢᵢᵢE₄ | AlkLEᵥᵢᵢ | E₄*E₅ | |
| 2680 | E_{1b}E₂E₃EᵢᵢᵢE₄ | AlkLEᵥᵢᵢ | E₄*E₅ | |
| 2681 | E₁ₐE₂Eᵢᵥ | AlkLEᵥᵢᵢ | E₄*E₅ | |
| 2682 | E_{1b}E₂Eᵢᵥ | AlkLEᵥᵢᵢ | E₄*E₅ | |
| 2683 | E₁ₐE₂E₃Eᵢᵥ | AlkLEᵥᵢᵢ | E₄*E₅ | |
| 2684 | E_{1b}E₂E₃Eᵢᵥ | AlkLEᵥᵢᵢ | E₄*E₅ | |
| 2685 | E₁ₐE₂EᵢᵥE₄ | AlkLEᵥᵢᵢ | E₄*E₅ | |
| 2686 | E_{1b}E₂EᵢᵥE₄ | AlkLEᵥᵢᵢ | E₄*E₅ | |
| 2687 | E₁ₐE₂E₃EᵢᵥE₄ | AlkLEᵥᵢᵢ | E₄*E₅ | |
| 2688 | E_{1b}E₂E₃EᵢᵥE₄ | AlkLEᵥᵢᵢ | E₄*E₅ | |
| 2689 | E₁ₐE₂ | Eᵥᵢᵢ | | Eₐ |
| 2690 | E_{1b}E₂ | Eᵥᵢᵢ | | Eₐ |
| 2691 | E₁ₐE₂E₃ | Eᵥᵢᵢ | | Eₐ |
| 2692 | E_{1b}E₂E₃ | Eᵥᵢᵢ | | Eₐ |
| 2693 | E₁ₐE₂Eᵢ | Eᵥᵢᵢ | | Eₐ |
| 2694 | E_{1b}E₂Eᵢ | Eᵥᵢᵢ | | Eₐ |
| 2695 | E₁ₐE₂Eᵢᵢ | Eᵥᵢᵢ | | Eₐ |
| 2696 | E_{1b}E₂Eᵢᵢ | Eᵥᵢᵢ | | Eₐ |
| 2697 | E₁ₐE₂EᵢᵢE_{iib} | Eᵥᵢᵢ | | Eₐ |
| 2698 | E_{1b}E₂EᵢᵢE_{iib} | Eᵥᵢᵢ | | Eₐ |
| 2699 | E₁ₐE₂E₃Eᵢ | Eᵥᵢᵢ | | Eₐ |
| 2700 | E_{1b}E₂E₃Eᵢ | Eᵥᵢᵢ | | Eₐ |
| 2701 | E₁ₐE₂E₃Eᵢᵢ | Eᵥᵢᵢ | | Eₐ |
| 2702 | E_{1b}E₂E₃Eᵢᵢ | Eᵥᵢᵢ | | Eₐ |
| 2703 | E₁ₐE₂E₃EᵢᵢE_{iib} | Eᵥᵢᵢ | | Eₐ |
| 2704 | E_{1b}E₂E₃EᵢᵢE_{iib} | Eᵥᵢᵢ | | Eₐ |
| 2705 | E₁ₐE₂E₄ | Eᵥᵢᵢ | | Eₐ |
| 2706 | E_{1b}E₂E₄ | Eᵥᵢᵢ | | Eₐ |
| 2707 | E₁ₐE₂E₃E₄ | Eᵥᵢᵢ | | Eₐ |
| 2708 | E_{1b}E₂E₃E₄ | Eᵥᵢᵢ | | Eₐ |
| 2709 | E₁ₐE₂EᵢE₄ | Eᵥᵢᵢ | | Eₐ |
| 2710 | E_{1b}E₂EᵢE₄ | Eᵥᵢᵢ | | Eₐ |
| 2711 | E₁ₐE₂EᵢᵢE₄ | Eᵥᵢᵢ | | Eₐ |
| 2712 | E_{1b}E₂EᵢᵢE₄ | Eᵥᵢᵢ | | Eₐ |
| 2713 | E₁ₐE₂EᵢᵢE_{iib}E₄ | Eᵥᵢᵢ | | Eₐ |
| 2714 | E_{1b}E₂EᵢᵢE_{iib}E₄ | Eᵥᵢᵢ | | Eₐ |
| 2715 | E₁ₐE₂E₃EᵢE₄ | Eᵥᵢᵢ | | Eₐ |
| 2716 | E_{1b}E₂E₃EᵢE₄ | Eᵥᵢᵢ | | Eₐ |
| 2717 | E₁ₐE₂E₃EᵢᵢE₄ | Eᵥᵢᵢ | | Eₐ |
| 2718 | E_{1b}E₂E₃EᵢᵢE₄ | Eᵥᵢᵢ | | Eₐ |
| 2719 | E₁ₐE₂E₃EᵢᵢE_{iib}E₄ | Eᵥᵢᵢ | | Eₐ |
| 2720 | E_{1b}E₂E₃EᵢᵢE_{iib}E₄ | Eᵥᵢᵢ | | Eₐ |
| 2721 | E₁ₐE₂Eᵢᵢᵢ | Eᵥᵢᵢ | | Eₐ |
| 2722 | E_{1b}E₂Eᵢᵢᵢ | Eᵥᵢᵢ | | Eₐ |
| 2723 | E₁ₐE₂E₃Eᵢᵢᵢ | Eᵥᵢᵢ | | Eₐ |
| 2724 | E_{1b}E₂E₃Eᵢᵢᵢ | Eᵥᵢᵢ | | Eₐ |
| 2725 | E₁ₐE₂EᵢᵢᵢE₄ | Eᵥᵢᵢ | | Eₐ |
| 2726 | E_{1b}E₂EᵢᵢᵢE₄ | Eᵥᵢᵢ | | Eₐ |
| 2727 | E₁ₐE₂E₃EᵢᵢᵢE₄ | Eᵥᵢᵢ | | Eₐ |
| 2728 | E_{1b}E₂E₃EᵢᵢᵢE₄ | Eᵥᵢᵢ | | Eₐ |
| 2729 | E₁ₐE₂Eᵢᵥ | Eᵥᵢᵢ | | Eₐ |
| 2730 | E_{1b}E₂Eᵢᵥ | Eᵥᵢᵢ | | Eₐ |
| 2731 | E₁ₐE₂E₃Eᵢᵥ | Eᵥᵢᵢ | | Eₐ |
| 2732 | E_{1b}E₂E₃Eᵢᵥ | Eᵥᵢᵢ | | Eₐ |
| 2733 | E₁ₐE₂EᵢᵥE₄ | Eᵥᵢᵢ | | Eₐ |
| 2734 | E_{1b}E₂EᵢᵥE₄ | Eᵥᵢᵢ | | Eₐ |
| 2735 | E₁ₐE₂E₃EᵢᵥE₄ | Eᵥᵢᵢ | | Eₐ |
| 2736 | E_{1b}E₂E₃EᵢᵥE₄ | Eᵥᵢᵢ | | Eₐ |
| 2737 | E₁ₐE₂ | Eᵥᵢᵢ | E₄* | Eₐ |
| 2738 | E_{1b}E₂ | Eᵥᵢᵢ | E₄* | Eₐ |
| 2739 | E₁ₐE₂E₃ | Eᵥᵢᵢ | E₄* | Eₐ |
| 2740 | E_{1b}E₂E₃ | Eᵥᵢᵢ | E₄* | Eₐ |
| 2741 | E₁ₐE₂Eᵢ | Eᵥᵢᵢ | E₄* | Eₐ |
| 2742 | E_{1b}E₂Eᵢ | Eᵥᵢᵢ | E₄* | Eₐ |
| 2743 | E₁ₐE₂Eᵢᵢ | Eᵥᵢᵢ | E₄* | Eₐ |
| 2744 | E_{1b}E₂Eᵢᵢ | Eᵥᵢᵢ | E₄* | Eₐ |
| 2745 | E₁ₐE₂EᵢᵢE_{iib} | Eᵥᵢᵢ | E₄* | Eₐ |
| 2746 | E_{1b}E₂EᵢᵢE_{iib} | Eᵥᵢᵢ | E₄* | Eₐ |
| 2747 | E₁ₐE₂E₃Eᵢ | Eᵥᵢᵢ | E₄* | Eₐ |
| 2748 | E_{1b}E₂E₃Eᵢ | Eᵥᵢᵢ | E₄* | Eₐ |
| 2749 | E₁ₐE₂E₃Eᵢᵢ | Eᵥᵢᵢ | E₄* | Eₐ |
| 2750 | E_{1b}E₂E₃Eᵢᵢ | Eᵥᵢᵢ | E₄* | Eₐ |
| 2751 | E₁ₐE₂E₃EᵢᵢE_{iib} | Eᵥᵢᵢ | E₄* | Eₐ |
| 2752 | E_{1b}E₂E₃EᵢᵢE_{iib} | Eᵥᵢᵢ | E₄* | Eₐ |
| 2753 | E₁ₐE₂E₄ | Eᵥᵢᵢ | E₄* | Eₐ |
| 2754 | E_{1b}E₂E₄ | Eᵥᵢᵢ | E₄* | Eₐ |
| 2755 | E₁ₐE₂E₃E₄ | Eᵥᵢᵢ | E₄* | Eₐ |
| 2756 | E_{1b}E₂E₃E₄ | Eᵥᵢᵢ | E₄* | Eₐ |
| 2757 | E₁ₐE₂EᵢE₄ | Eᵥᵢᵢ | E₄* | Eₐ |
| 2758 | E_{1b}E₂EᵢE₄ | Eᵥᵢᵢ | E₄* | Eₐ |
| 2759 | E₁ₐE₂EᵢᵢE₄ | Eᵥᵢᵢ | E₄* | Eₐ |
| 2760 | E_{1b}E₂EᵢᵢE₄ | Eᵥᵢᵢ | E₄* | Eₐ |
| 2761 | E₁ₐE₂EᵢᵢE_{iib}E₄ | Eᵥᵢᵢ | E₄* | Eₐ |
| 2762 | E_{1b}E₂EᵢᵢE_{iib}E₄ | Eᵥᵢᵢ | E₄* | Eₐ |
| 2763 | E₁ₐE₂E₃EᵢE₄ | Eᵥᵢᵢ | E₄* | Eₐ |
| 2764 | E_{1b}E₂E₃EᵢE₄ | Eᵥᵢᵢ | E₄* | Eₐ |
| 2765 | E₁ₐE₂E₃EᵢᵢE₄ | Eᵥᵢᵢ | E₄* | Eₐ |
| 2766 | E_{1b}E₂E₃EᵢᵢE₄ | Eᵥᵢᵢ | E₄* | Eₐ |
| 2767 | E₁ₐE₂E₃EᵢᵢE_{iib}E₄ | Eᵥᵢᵢ | E₄* | Eₐ |
| 2768 | E_{1b}E₂E₃EᵢᵢE_{iib}E₄ | Eᵥᵢᵢ | E₄* | Eₐ |
| 2769 | E₁ₐE₂Eᵢᵢᵢ | Eᵥᵢᵢ | E₄* | Eₐ |
| 2770 | E_{1b}E₂Eᵢᵢᵢ | Eᵥᵢᵢ | E₄* | Eₐ |
| 2771 | E₁ₐE₂E₃Eᵢᵢᵢ | Eᵥᵢᵢ | E₄* | Eₐ |
| 2772 | E_{1b}E₂E₃Eᵢᵢᵢ | Eᵥᵢᵢ | E₄* | Eₐ |
| 2773 | E₁ₐE₂EᵢᵢᵢE₄ | Eᵥᵢᵢ | E₄* | Eₐ |
| 2774 | E_{1b}E₂EᵢᵢᵢE₄ | Eᵥᵢᵢ | E₄* | Eₐ |
| 2775 | E₁ₐE₂E₃EᵢᵢᵢE₄ | Eᵥᵢᵢ | E₄* | Eₐ |
| 2776 | E_{1b}E₂E₃EᵢᵢᵢE₄ | Eᵥᵢᵢ | E₄* | Eₐ |
| 2777 | E₁ₐE₂Eᵢᵥ | Eᵥᵢᵢ | E₄* | Eₐ |
| 2778 | E_{1b}E₂Eᵢᵥ | Eᵥᵢᵢ | E₄* | Eₐ |
| 2779 | E₁ₐE₂E₃Eᵢᵥ | Eᵥᵢᵢ | E₄* | Eₐ |
| 2780 | E_{1b}E₂E₃Eᵢᵥ | Eᵥᵢᵢ | E₄* | Eₐ |
| 2781 | E₁ₐE₂EᵢᵥE₄ | Eᵥᵢᵢ | E₄* | Eₐ |
| 2782 | E_{1b}E₂EᵢᵥE₄ | Eᵥᵢᵢ | E₄* | Eₐ |
| 2783 | E₁ₐE₂E₃EᵢᵥE₄ | Eᵥᵢᵢ | E₄* | Eₐ |
| 2784 | E_{1b}E₂E₃EᵢᵥE₄ | Eᵥᵢᵢ | E₄* | Eₐ |
| 2785 | E₁ₐE₂ | Eᵥᵢᵢ | E₅ | Eₐ |
| 2786 | E_{1b}E₂ | Eᵥᵢᵢ | E₅ | Eₐ |
| 2787 | E₁ₐE₂E₃ | Eᵥᵢᵢ | E₅ | Eₐ |
| 2788 | E_{1b}E₂E₃ | Eᵥᵢᵢ | E₅ | Eₐ |
| 2789 | E₁ₐE₂Eᵢ | Eᵥᵢᵢ | E₅ | Eₐ |
| 2790 | E_{1b}E₂Eᵢ | Eᵥᵢᵢ | E₅ | Eₐ |
| 2791 | E₁ₐE₂Eᵢᵢ | Eᵥᵢᵢ | E₅ | Eₐ |
| 2792 | E_{1b}E₂Eᵢᵢ | Eᵥᵢᵢ | E₅ | Eₐ |
| 2793 | E₁ₐE₂EᵢᵢE_{iib} | Eᵥᵢᵢ | E₅ | Eₐ |
| 2794 | E_{1b}E₂EᵢᵢE_{iib} | Eᵥᵢᵢ | E₅ | Eₐ |
| 2795 | E₁ₐE₂E₃Eᵢ | Eᵥᵢᵢ | E₅ | Eₐ |
| 2796 | E_{1b}E₂E₃Eᵢ | Eᵥᵢᵢ | E₅ | Eₐ |
| 2797 | E₁ₐE₂E₃Eᵢᵢ | Eᵥᵢᵢ | E₅ | Eₐ |
| 2798 | E_{1b}E₂E₃Eᵢᵢ | Eᵥᵢᵢ | E₅ | Eₐ |
| 2799 | E₁ₐE₂E₃EᵢᵢE_{iib} | Eᵥᵢᵢ | E₅ | Eₐ |
| 2800 | E_{1b}E₂E₃EᵢᵢE_{iib} | Eᵥᵢᵢ | E₅ | Eₐ |
| 2801 | E₁ₐE₂E₄ | Eᵥᵢᵢ | E₅ | Eₐ |
| 2802 | E_{1b}E₂E₄ | Eᵥᵢᵢ | E₅ | Eₐ |
| 2803 | E₁ₐE₂E₃E₄ | Eᵥᵢᵢ | E₅ | Eₐ |
| 2804 | E_{1b}E₂E₃E₄ | Eᵥᵢᵢ | E₅ | Eₐ |
| 2805 | E₁ₐE₂EᵢE₄ | Eᵥᵢᵢ | E₅ | Eₐ |
| 2806 | E_{1b}E₂EᵢE₄ | Eᵥᵢᵢ | E₅ | Eₐ |
| 2807 | E₁ₐE₂EᵢᵢE₄ | Eᵥᵢᵢ | E₅ | Eₐ |
| 2808 | E_{1b}E₂EᵢᵢE₄ | Eᵥᵢᵢ | E₅ | Eₐ |
| 2809 | E₁ₐE₂EᵢᵢE_{iib}E₄ | Eᵥᵢᵢ | E₅ | Eₐ |
| 2810 | E_{1b}E₂EᵢᵢE_{iib}E₄ | Eᵥᵢᵢ | E₅ | Eₐ |
| 2811 | E₁ₐE₂E₃EᵢE₄ | Eᵥᵢᵢ | E₅ | Eₐ |
| 2812 | E_{1b}E₂E₃EᵢE₄ | Eᵥᵢᵢ | E₅ | Eₐ |
| 2813 | E₁ₐE₂E₃EᵢᵢE₄ | Eᵥᵢᵢ | E₅ | Eₐ |
| 2814 | E_{1b}E₂E₃EᵢᵢE₄ | Eᵥᵢᵢ | E₅ | Eₐ |
| 2815 | E₁ₐE₂E₃EᵢᵢE_{iib}E₄ | Eᵥᵢᵢ | E₅ | Eₐ |
| 2816 | E_{1b}E₂E₃EᵢᵢE_{iib}E₄ | Eᵥᵢᵢ | E₅ | Eₐ |
| 2817 | E₁ₐE₂Eᵢᵢᵢ | Eᵥᵢᵢ | E₅ | Eₐ |
| 2818 | E_{1b}E₂Eᵢᵢᵢ | Eᵥᵢᵢ | E₅ | Eₐ |
| 2819 | E₁ₐE₂E₃Eᵢᵢᵢ | Eᵥᵢᵢ | E₅ | Eₐ |
| 2820 | E_{1b}E₂E₃Eᵢᵢᵢ | Eᵥᵢᵢ | E₅ | Eₐ |
| 2821 | E₁ₐE₂EᵢᵢᵢE₄ | Eᵥᵢᵢ | E₅ | Eₐ |
| 2822 | E_{1b}E₂EᵢᵢᵢE₄ | Eᵥᵢᵢ | E₅ | Eₐ |
| 2823 | E₁ₐE₂E₃EᵢᵢᵢE₄ | Eᵥᵢᵢ | E₅ | Eₐ |
| 2824 | E_{1b}E₂E₃EᵢᵢᵢE₄ | Eᵥᵢᵢ | E₅ | Eₐ |
| 2825 | E₁ₐE₂Eᵢᵥ | Eᵥᵢᵢ | E₅ | Eₐ |
| 2826 | E_{1b}E₂Eᵢᵥ | Eᵥᵢᵢ | E₅ | Eₐ |
| 2827 | E₁ₐE₂E₃Eᵢᵥ | Eᵥᵢᵢ | E₅ | Eₐ |
| 2828 | E_{1b}E₂E₃Eᵢᵥ | Eᵥᵢᵢ | E₅ | Eₐ |
| 2829 | E₁ₐE₂EᵢᵥE₄ | Eᵥᵢᵢ | E₅ | Eₐ |
| 2830 | E_{1b}E₂EᵢᵥE₄ | Eᵥᵢᵢ | E₅ | Eₐ |
| 2831 | E₁ₐE₂E₃EᵢᵥE₄ | Eᵥᵢᵢ | E₅ | Eₐ |
| 2832 | E_{1b}E₂E₃EᵢᵥE₄ | Eᵥᵢᵢ | E₅ | Eₐ |
| 2833 | E₁ₐE₂ | Eᵥᵢᵢ | E₄*E₅ | Eₐ |
| 2834 | E_{1b}E₂ | Eᵥᵢᵢ | E₄*E₅ | Eₐ |
| 2835 | E₁ₐE₂E₃ | Eᵥᵢᵢ | E₄*E₅ | Eₐ |
| 2836 | E_{1b}E₂E₃ | Eᵥᵢᵢ | E₄*E₅ | Eₐ |
| 2837 | E₁ₐE₂Eᵢ | Eᵥᵢᵢ | E₄*E₅ | Eₐ |
| 2838 | E_{1b}E₂Eᵢ | Eᵥᵢᵢ | E₄*E₅ | Eₐ |
| 2839 | E₁ₐE₂Eᵢᵢ | Eᵥᵢᵢ | E₄*E₅ | Eₐ |
| 2840 | E_{1b}E₂Eᵢᵢ | Eᵥᵢᵢ | E₄*E₅ | Eₐ |
| 2841 | E₁ₐE₂EᵢᵢE_{iib} | Eᵥᵢᵢ | E₄*E₅ | Eₐ |
| 2842 | E_{1b}E₂EᵢᵢE_{iib} | Eᵥᵢᵢ | E₄*E₅ | Eₐ |
| 2843 | E₁ₐE₂E₃Eᵢ | Eᵥᵢᵢ | E₄*E₅ | Eₐ |
| 2844 | E_{1b}E₂E₃Eᵢ | Eᵥᵢᵢ | E₄*E₅ | Eₐ |
| 2845 | E₁ₐE₂E₃Eᵢᵢ | Eᵥᵢᵢ | E₄*E₅ | Eₐ |
| 2846 | E_{1b}E₂E₃Eᵢᵢ | Eᵥᵢᵢ | E₄*E₅ | Eₐ |
| 2847 | E₁ₐE₂E₃EᵢᵢE_{iib} | Eᵥᵢᵢ | E₄*E₅ | Eₐ |
| 2848 | E_{1b}E₂E₃EᵢᵢE_{iib} | Eᵥᵢᵢ | E₄*E₅ | Eₐ |
| 2849 | E₁ₐE₂E₄ | Eᵥᵢᵢ | E₄*E₅ | Eₐ |
| 2850 | E_{1b}E₂E₄ | Eᵥᵢᵢ | E₄*E₅ | Eₐ |
| 2851 | E₁ₐE₂E₃E₄ | Eᵥᵢᵢ | E₄*E₅ | Eₐ |
| 2852 | E_{1b}E₂E₃E₄ | Eᵥᵢᵢ | E₄*E₅ | Eₐ |
| 2853 | E₁ₐE₂EᵢE₄ | Eᵥᵢᵢ | E₄*E₅ | Eₐ |
| 2854 | E_{1b}E₂EᵢE₄ | Eᵥᵢᵢ | E₄*E₅ | Eₐ |
| 2855 | E₁ₐE₂EᵢᵢE₄ | Eᵥᵢᵢ | E₄*E₅ | Eₐ |
| 2856 | E_{1b}E₂EᵢᵢE₄ | Eᵥᵢᵢ | E₄*E₅ | Eₐ |
| 2857 | E₁ₐE₂EᵢᵢE_{iib}E₄ | Eᵥᵢᵢ | E₄*E₅ | Eₐ |
| 2858 | E_{1b}E₂EᵢᵢE_{iib}E₄ | Eᵥᵢᵢ | E₄*E₅ | Eₐ |
| 2859 | E₁ₐE₂E₃EᵢE₄ | Eᵥᵢᵢ | E₄*E₅ | Eₐ |
| 2860 | E_{1b}E₂E₃EᵢE₄ | Eᵥᵢᵢ | E₄*E₅ | Eₐ |
| 2861 | E₁ₐE₂E₃EᵢᵢE₄ | Eᵥᵢᵢ | E₄*E₅ | Eₐ |
| 2862 | E_{1b}E₂E₃EᵢᵢE₄ | Eᵥᵢᵢ | E₄*E₅ | Eₐ |
| 2863 | E₁ₐE₂E₃EᵢᵢE_{iib}E₄ | Eᵥᵢᵢ | E₄*E₅ | Eₐ |
| 2864 | E_{1b}E₂E₃EᵢᵢE_{iib}E₄ | Eᵥᵢᵢ | E₄*E₅ | Eₐ |
| 2865 | E₁ₐE₂Eᵢᵢᵢ | Eᵥᵢᵢ | E₄*E₅ | Eₐ |
| 2866 | E_{1b}E₂Eᵢᵢᵢ | Eᵥᵢᵢ | E₄*E₅ | Eₐ |
| 2867 | E₁ₐE₂E₃Eᵢᵢᵢ | Eᵥᵢᵢ | E₄*E₅ | Eₐ |
| 2868 | E_{1b}E₂E₃Eᵢᵢᵢ | Eᵥᵢᵢ | E₄*E₅ | Eₐ |
| 2869 | E₁ₐE₂EᵢᵢᵢE₄ | Eᵥᵢᵢ | E₄*E₅ | Eₐ |
| 2870 | E_{1b}E₂EᵢᵢᵢE₄ | Eᵥᵢᵢ | E₄*E₅ | Eₐ |
| 2871 | E₁ₐE₂E₃EᵢᵢᵢE₄ | Eᵥᵢᵢ | E₄*E₅ | Eₐ |
| 2872 | E_{1b}E₂E₃EᵢᵢᵢE₄ | Eᵥᵢᵢ | E₄*E₅ | Eₐ |
| 2873 | E₁ₐE₂Eᵢᵥ | Eᵥᵢᵢ | E₄*E₅ | Eₐ |
| 2874 | E_{1b}E₂Eᵢᵥ | Eᵥᵢᵢ | E₄*E₅ | Eₐ |
| 2875 | E₁ₐE₂E₃Eᵢᵥ | Eᵥᵢᵢ | E₄*E₅ | Eₐ |
| 2876 | E_{1b}E₂E₃Eᵢᵥ | Eᵥᵢᵢ | E₄*E₅ | Eₐ |
| 2877 | E₁ₐE₂EᵢᵥE₄ | Eᵥᵢᵢ | E₄*E₅ | Eₐ |
| 2878 | E_{1b}E₂EᵢᵥE₄ | Eᵥᵢᵢ | E₄*E₅ | Eₐ |
| 2879 | E₁ₐE₂E₃EᵢᵥE₄ | Eᵥᵢᵢ | E₄*E₅ | Eₐ |
| 2880 | E_{1b}E₂E₃EᵢᵥE₄ | Eᵥᵢᵢ | E₄*E₅ | Eₐ |
| 2881 | E₁ₐE₂ | AlkLEᵥᵢᵢ | | Eₐ |
| 2882 | E_{1b}E₂ | AlkLEᵥᵢᵢ | | Eₐ |
| 2883 | E₁ₐE₂E₃ | AlkLEᵥᵢᵢ | | Eₐ |
| 2884 | E_{1b}E₂E₃ | AlkLEᵥᵢᵢ | | Eₐ |
| 2885 | E₁ₐE₂Eᵢ | AlkLEᵥᵢᵢ | | Eₐ |
| 2886 | E_{1b}E₂Eᵢ | AlkLEᵥᵢᵢ | | Eₐ |
| 2887 | E₁ₐE₂Eᵢᵢ | AlkLEᵥᵢᵢ | | Eₐ |
| 2888 | E_{1b}E₂Eᵢᵢ | AlkLEᵥᵢᵢ | | Eₐ |
| 2889 | E₁ₐE₂EᵢᵢE_{iib} | AlkLEᵥᵢᵢ | | Eₐ |
| 2890 | E_{1b}E₂EᵢᵢE_{iib} | AlkLEᵥᵢᵢ | | Eₐ |
| 2891 | E₁ₐE₂E₃Eᵢ | AlkLEᵥᵢᵢ | | Eₐ |
| 2892 | E_{1b}E₂E₃Eᵢ | AlkLEᵥᵢᵢ | | Eₐ |
| 2893 | E₁ₐE₂E₃Eᵢᵢ | AlkLEᵥᵢᵢ | | Eₐ |
| 2894 | E_{1b}E₂E₃Eᵢᵢ | AlkLEᵥᵢᵢ | | Eₐ |
| 2895 | E₁ₐE₂E₃EᵢᵢE_{iib} | AlkLEᵥᵢᵢ | | Eₐ |
| 2896 | E_{1b}E₂E₃EᵢᵢE_{iib} | AlkLEᵥᵢᵢ | | Eₐ |
| 2897 | E₁ₐE₂E₄ | AlkLEᵥᵢᵢ | | Eₐ |
| 2898 | E_{1b}E₂E₄ | AlkLEᵥᵢᵢ | | Eₐ |
| 2899 | E₁ₐE₂E₃E₄ | AlkLEᵥᵢᵢ | | Eₐ |
| 2900 | E_{1b}E₂E₃E₄ | AlkLEᵥᵢᵢ | | Eₐ |
| 2901 | E₁ₐE₂EᵢE₄ | AlkLEᵥᵢᵢ | | Eₐ |
| 2902 | E_{1b}E₂EᵢE₄ | AlkLEᵥᵢᵢ | | Eₐ |
| 2903 | E₁ₐE₂EᵢᵢE₄ | AlkLEᵥᵢᵢ | | Eₐ |
| 2904 | E_{1b}E₂EᵢᵢE₄ | AlkLEᵥᵢᵢ | | Eₐ |
| 2905 | E₁ₐE₂EᵢᵢE_{iib}E₄ | AlkLEᵥᵢᵢ | | Eₐ |
| 2906 | E_{1b}E₂EᵢᵢE_{iib}E₄ | AlkLEᵥᵢᵢ | | Eₐ |
| 2907 | E₁ₐE₂E₃EᵢE₄ | AlkLEᵥᵢᵢ | | Eₐ |
| 2908 | E_{1b}E₂E₃EᵢE₄ | AlkLEᵥᵢᵢ | | Eₐ |
| 2909 | E₁ₐE₂E₃EᵢᵢE₄ | AlkLEᵥᵢᵢ | | Eₐ |
| 2910 | E_{1b}E₂E₃EᵢᵢE₄ | AlkLEᵥᵢᵢ | | Eₐ |
| 2911 | E₁ₐE₂E₃EᵢᵢE_{iib}E₄ | AlkLEᵥᵢᵢ | | Eₐ |
| 2912 | E_{1b}E₂E₃EᵢᵢE_{iib}E₄ | AlkLEᵥᵢᵢ | | Eₐ |
| 2913 | E₁ₐE₂Eᵢᵢᵢ | AlkLEᵥᵢᵢ | | Eₐ |
| 2914 | E_{1b}E₂Eᵢᵢᵢ | AlkLEᵥᵢᵢ | | Eₐ |
| 2915 | E₁ₐE₂E₃Eᵢᵢᵢ | AlkLEᵥᵢᵢ | | Eₐ |
| 2916 | E_{1b}E₂E₃Eᵢᵢᵢ | AlkLEᵥᵢᵢ | | Eₐ |
| 2917 | E₁ₐE₂EᵢᵢᵢE₄ | AlkLEᵥᵢᵢ | | Eₐ |
| 2918 | E_{1b}E₂EᵢᵢᵢE₄ | AlkLEᵥᵢᵢ | | Eₐ |
| 2919 | E₁ₐE₂E₃EᵢᵢᵢE₄ | AlkLEᵥᵢᵢ | | Eₐ |
| 2920 | E_{1b}E₂E₃EᵢᵢᵢE₄ | AlkLEᵥᵢᵢ | | Eₐ |
| 2921 | E₁ₐE₂Eᵢᵥ | AlkLEᵥᵢᵢ | | Eₐ |
| 2922 | E_{1b}E₂Eᵢᵥ | AlkLEᵥᵢᵢ | | Eₐ |
| 2923 | E₁ₐE₂E₃Eᵢᵥ | AlkLEᵥᵢᵢ | | Eₐ |
| 2924 | E_{1b}E₂E₃Eᵢᵥ | AlkLEᵥᵢᵢ | | Eₐ |
| 2925 | E₁ₐE₂EᵢᵥE₄ | AlkLEᵥᵢᵢ | | Eₐ |
| 2926 | E_{1b}E₂EᵢᵥE₄ | AlkLEᵥᵢᵢ | | Eₐ |
| 2927 | E₁ₐE₂E₃EᵢᵥE₄ | AlkLEᵥᵢᵢ | | Eₐ |
| 2928 | E_{1b}E₂E₃EᵢᵥE₄ | AlkLEᵥᵢᵢ | | Eₐ |
| 2929 | E₁ₐE₂ | AlkLEᵥᵢᵢ | E₄* | Eₐ |
| 2930 | E_{1b}E₂ | AlkLEᵥᵢᵢ | E₄* | Eₐ |
| 2931 | E₁ₐE₂E₃ | AlkLEᵥᵢᵢ | E₄* | Eₐ |
| 2932 | E_{1b}E₂E₃ | AlkLEᵥᵢᵢ | E₄* | Eₐ |
| 2933 | E₁ₐE₂Eᵢ | AlkLEᵥᵢᵢ | E₄* | Eₐ |
| 2934 | E_{1b}E₂Eᵢ | AlkLEᵥᵢᵢ | E₄* | Eₐ |
| 2935 | E₁ₐE₂Eᵢᵢ | AlkLEᵥᵢᵢ | E₄* | Eₐ |
| 2936 | E_{1b}E₂Eᵢᵢ | AlkLEᵥᵢᵢ | E₄* | Eₐ |
| 2937 | E₁ₐE₂EᵢᵢE_{iib} | AlkLEᵥᵢᵢ | E₄* | Eₐ |
| 2938 | E_{1b}E₂EᵢᵢE_{iib} | AlkLEᵥᵢᵢ | E₄* | Eₐ |
| 2939 | E₁ₐE₂E₃Eᵢ | AlkLEᵥᵢᵢ | E₄* | Eₐ |
| 2940 | E_{1b}E₂E₃Eᵢ | AlkLEᵥᵢᵢ | E₄* | Eₐ |
| 2941 | E₁ₐE₂E₃Eᵢᵢ | AlkLEᵥᵢᵢ | E₄* | Eₐ |
| 2942 | E_{1b}E₂E₃Eᵢᵢ | AlkLEᵥᵢᵢ | E₄* | Eₐ |
| 2943 | E₁ₐE₂E₃EᵢᵢE_{iib} | AlkLEᵥᵢᵢ | E₄* | Eₐ |
| 2944 | E_{1b}E₂E₃EᵢᵢE_{iib} | AlkLEᵥᵢᵢ | E₄* | Eₐ |
| 2945 | E₁ₐE₂E₄ | AlkLEᵥᵢᵢ | E₄* | Eₐ |
| 2946 | E_{1b}E₂E₄ | AlkLEᵥᵢᵢ | E₄* | Eₐ |
| 2947 | E₁ₐE₂E₃E₄ | AlkLEᵥᵢᵢ | E₄* | Eₐ |
| 2948 | E_{1b}E₂E₃E₄ | AlkLEᵥᵢᵢ | E₄* | Eₐ |
| 2949 | E₁ₐE₂EᵢE₄ | AlkLEᵥᵢᵢ | E₄* | Eₐ |
| 2950 | E_{1b}E₂EᵢE₄ | AlkLEᵥᵢᵢ | E₄* | Eₐ |
| 2951 | E₁ₐE₂EᵢᵢE₄ | AlkLEᵥᵢᵢ | E₄* | Eₐ |
| 2952 | E_{1b}E₂EᵢᵢE₄ | AlkLEᵥᵢᵢ | E₄* | Eₐ |
| 2953 | E₁ₐE₂EᵢᵢE_{iib}E₄ | AlkLEᵥᵢᵢ | E₄* | Eₐ |
| 2954 | E_{1b}E₂EᵢᵢE_{iib}E₄ | AlkLEᵥᵢᵢ | E₄* | Eₐ |
| 2955 | E₁ₐE₂E₃EᵢE₄ | AlkLEᵥᵢᵢ | E₄* | Eₐ |
| 2956 | E_{1b}E₂E₃EᵢE₄ | AlkLEᵥᵢᵢ | E₄* | Eₐ |
| 2957 | E₁ₐE₂E₃EᵢᵢE₄ | AlkLEᵥᵢᵢ | E₄* | Eₐ |
| 2958 | E_{1b}E₂E₃EᵢᵢE₄ | AlkLEᵥᵢᵢ | E₄* | Eₐ |
| 2959 | E₁ₐE₂E₃EᵢᵢE_{iib}E₄ | AlkLEᵥᵢᵢ | E₄* | Eₐ |
| 2960 | E_{1b}E₂E₃EᵢᵢE_{iib}E₄ | AlkLEᵥᵢᵢ | E₄* | Eₐ |
| 2961 | E₁ₐE₂Eᵢᵢᵢ | AlkLEᵥᵢᵢ | E₄* | Eₐ |
| 2962 | E_{1b}E₂Eᵢᵢᵢ | AlkLEᵥᵢᵢ | E₄* | Eₐ |
| 2963 | E₁ₐE₂E₃Eᵢᵢᵢ | AlkLEᵥᵢᵢ | E₄* | Eₐ |
| 2964 | E_{1b}E₂E₃Eᵢᵢᵢ | AlkLEᵥᵢᵢ | E₄* | Eₐ |
| 2965 | E₁ₐE₂EᵢᵢᵢE₄ | AlkLEᵥᵢᵢ | E₄* | Eₐ |
| 2966 | E_{1b}E₂EᵢᵢᵢE₄ | AlkLEᵥᵢᵢ | E₄* | Eₐ |
| 2967 | E₁ₐE₂E₃EᵢᵢᵢE₄ | AlkLEᵥᵢᵢ | E₄* | Eₐ |
| 2968 | E_{1b}E₂E₃EᵢᵢᵢE₄ | AlkLEᵥᵢᵢ | E₄* | Eₐ |
| 2969 | E₁ₐE₂Eᵢᵥ | AlkLEᵥᵢᵢ | E₄* | Eₐ |
| 2970 | E_{1b}E₂Eᵢᵥ | AlkLEᵥᵢᵢ | E₄* | Eₐ |
| 2971 | E₁ₐE₂E₃Eᵢᵥ | AlkLEᵥᵢᵢ | E₄* | Eₐ |
| 2972 | E_{1b}E₂E₃Eᵢᵥ | AlkLEᵥᵢᵢ | E₄* | Eₐ |
| 2973 | E₁ₐE₂EᵢᵥE₄ | AlkLEᵥᵢᵢ | E₄* | Eₐ |
| 2974 | E_{1b}E₂EᵢᵥE₄ | AlkLEᵥᵢᵢ | E₄* | Eₐ |
| 2975 | E₁ₐE₂E₃EᵢᵥE₄ | AlkLEᵥᵢᵢ | E₄* | Eₐ |
| 2976 | E_{1b}E₂E₃EᵢᵥE₄ | AlkLEᵥᵢᵢ | E₄* | Eₐ |
| 2977 | E₁ₐE₂ | AlkLEᵥᵢᵢ | E₅ | Eₐ |
| 2978 | E_{1b}E₂ | AlkLEᵥᵢᵢ | E₅ | Eₐ |
| 2979 | E₁ₐE₂E₃ | AlkLEᵥᵢᵢ | E₅ | Eₐ |
| 2980 | E_{1b}E₂E₃ | AlkLEᵥᵢᵢ | E₅ | Eₐ |
| 2981 | E₁ₐE₂Eᵢ | AlkLEᵥᵢᵢ | E₅ | Eₐ |
| 2982 | E_{1b}E₂Eᵢ | AlkLEᵥᵢᵢ | E₅ | Eₐ |
| 2983 | E₁ₐE₂Eᵢᵢ | AlkLEᵥᵢᵢ | E₅ | Eₐ |
| 2984 | E_{1b}E₂Eᵢᵢ | AlkLEᵥᵢᵢ | E₅ | Eₐ |
| 2985 | E₁ₐE₂EᵢᵢE_{iib} | AlkLEᵥᵢᵢ | E₅ | Eₐ |
| 2986 | E_{1b}E₂EᵢᵢE_{iib} | AlkLEᵥᵢᵢ | E₅ | Eₐ |
| 2987 | E₁ₐE₂E₃Eᵢ | AlkLEᵥᵢᵢ | E₅ | Eₐ |
| 2988 | E_{1b}E₂E₃Eᵢ | AlkLEᵥᵢᵢ | E₅ | Eₐ |
| 2989 | E₁ₐE₂E₃Eᵢᵢ | AlkLEᵥᵢᵢ | E₅ | Eₐ |
| 2990 | E_{1b}E₂E₃Eᵢᵢ | AlkLEᵥᵢᵢ | E₅ | Eₐ |
| 2991 | E₁ₐE₂E₃EᵢᵢE_{iib} | AlkLEᵥᵢᵢ | E₅ | Eₐ |
| 2992 | E_{1b}E₂E₃EᵢᵢE_{iib} | AlkLEᵥᵢᵢ | E₅ | Eₐ |
| 2993 | E₁ₐE₂E₄ | AlkLEᵥᵢᵢ | E₅ | Eₐ |
| 2994 | E_{1b}E₂E₄ | AlkLEᵥᵢᵢ | E₅ | Eₐ |
| 2995 | E₁ₐE₂E₃E₄ | AlkLEᵥᵢᵢ | E₅ | Eₐ |
| 2996 | E_{1b}E₂E₃E₄ | AlkLEᵥᵢᵢ | E₅ | Eₐ |
| 2997 | E₁ₐE₂EᵢE₄ | AlkLEᵥᵢᵢ | E₅ | Eₐ |
| 2998 | E_{1b}E₂EᵢE₄ | AlkLEᵥᵢᵢ | E₅ | Eₐ |
| 2999 | E₁ₐE₂EᵢᵢE₄ | AlkLEᵥᵢᵢ | E₅ | Eₐ |
| 3000 | E_{1b}E₂EᵢᵢE₄ | AlkLEᵥᵢᵢ | E₅ | Eₐ |
| 3001 | E₁ₐE₂EᵢᵢE_{iib}E₄ | AlkLEᵥᵢᵢ | E₅ | Eₐ |
| 3002 | E_{1b}E₂EᵢᵢE_{iib}E₄ | AlkLEᵥᵢᵢ | E₅ | Eₐ |
| 3003 | E₁ₐE₂E₃EᵢE₄ | AlkLEᵥᵢᵢ | E₅ | Eₐ |
| 3004 | E_{1b}E₂E₃EᵢE₄ | AlkLEᵥᵢᵢ | E₅ | Eₐ |
| 3005 | E₁ₐE₂E₃EᵢᵢE₄ | AlkLEᵥᵢᵢ | E₅ | Eₐ |
| 3006 | E_{1b}E₂E₃EᵢᵢE₄ | AlkLEᵥᵢᵢ | E₅ | Eₐ |
| 3007 | E₁ₐE₂E₃EᵢᵢE_{iib}E₄ | AlkLEᵥᵢᵢ | E₅ | Eₐ |
| 3008 | E_{1b}E₂E₃EᵢᵢE_{iib}E₄ | AlkLEᵥᵢᵢ | E₅ | Eₐ |
| 3009 | E₁ₐE₂Eᵢᵢᵢ | AlkLEᵥᵢᵢ | E₅ | Eₐ |
| 3010 | E_{1b}E₂Eᵢᵢᵢ | AlkLEᵥᵢᵢ | E₅ | Eₐ |
| 3011 | E₁ₐE₂E₃Eᵢᵢᵢ | AlkLEᵥᵢᵢ | E₅ | Eₐ |
| 3012 | E_{1b}E₂E₃Eᵢᵢᵢ | AlkLEᵥᵢᵢ | E₅ | Eₐ |
| 3013 | E₁ₐE₂EᵢᵢᵢE₄ | AlkLEᵥᵢᵢ | E₅ | Eₐ |
| 3014 | E_{1b}E₂EᵢᵢᵢE₄ | AlkLEᵥᵢᵢ | E₅ | Eₐ |
| 3015 | E₁ₐE₂E₃EᵢᵢᵢE₄ | AlkLEᵥᵢᵢ | E₅ | Eₐ |
| 3016 | E_{1b}E₂E₃EᵢᵢᵢE₄ | AlkLEᵥᵢᵢ | E₅ | Eₐ |
| 3017 | E₁ₐE₂Eᵢᵥ | AlkLEᵥᵢᵢ | E₅ | Eₐ |
| 3018 | E_{1b}E₂Eᵢᵥ | AlkLEᵥᵢᵢ | E₅ | Eₐ |
| 3019 | E₁ₐE₂E₃Eᵢᵥ | AlkLEᵥᵢᵢ | E₅ | Eₐ |
| 3020 | E_{1b}E₂E₃Eᵢᵥ | AlkLEᵥᵢᵢ | E₅ | Eₐ |
| 3021 | E₁ₐE₂EᵢᵥE₄ | AlkLEᵥᵢᵢ | E₅ | Eₐ |
| 3022 | E_{1b}E₂EᵢᵥE₄ | AlkLEᵥᵢᵢ | E₅ | Eₐ |
| 3023 | E₁ₐE₂E₃EᵢᵥE₄ | AlkLEᵥᵢᵢ | E₅ | Eₐ |
| 3024 | E_{1b}E₂E₃EᵢᵥE₄ | AlkLEᵥᵢᵢ | E₅ | Eₐ |
| 3025 | E₁ₐE₂ | AlkLEᵥᵢᵢ | E₄*E₅ | Eₐ |
| 3026 | E_{1b}E₂ | AlkLEᵥᵢᵢ | E₄*E₅ | Eₐ |
| 3027 | E₁ₐE₂E₃ | AlkLEᵥᵢᵢ | E₄*E₅ | Eₐ |
| 3028 | E_{1b}E₂E₃ | AlkLEᵥᵢᵢ | E₄*E₅ | Eₐ |
| 3029 | E₁ₐE₂Eᵢ | AlkLEᵥᵢᵢ | E₄*E₅ | Eₐ |
| 3030 | E_{1b}E₂Eᵢ | AlkLEᵥᵢᵢ | E₄*E₅ | Eₐ |
| 3031 | E₁ₐE₂Eᵢᵢ | AlkLEᵥᵢᵢ | E₄*E₅ | Eₐ |
| 3032 | E_{1b}E₂Eᵢᵢ | AlkLEᵥᵢᵢ | E₄*E₅ | Eₐ |
| 3033 | E₁ₐE₂EᵢᵢE_{iib} | AlkLEᵥᵢᵢ | E₄*E₅ | Eₐ |
| 3034 | E_{1b}E₂EᵢᵢE_{iib} | AlkLEᵥᵢᵢ | E₄*E₅ | Eₐ |
| 3035 | E₁ₐE₂E₃Eᵢ | AlkLEᵥᵢᵢ | E₄*E₅ | Eₐ |
| 3036 | E_{1b}E₂E₃Eᵢ | AlkLEᵥᵢᵢ | E₄*E₅ | Eₐ |
| 3037 | E₁ₐE₂E₃Eᵢᵢ | AlkLEᵥᵢᵢ | E₄*E₅ | Eₐ |
| 3038 | E_{1b}E₂E₃Eᵢᵢ | AlkLEᵥᵢᵢ | E₄*E₅ | Eₐ |
| 3039 | E₁ₐE₂E₃EᵢᵢE_{iib} | AlkLEᵥᵢᵢ | E₄*E₅ | Eₐ |
| 3040 | E_{1b}E₂E₃EᵢᵢE_{iib} | AlkLEᵥᵢᵢ | E₄*E₅ | Eₐ |
| 3041 | E₁ₐE₂E₄ | AlkLEᵥᵢᵢ | E₄*E₅ | Eₐ |
| 3042 | E_{1b}E₂E₄ | AlkLEᵥᵢᵢ | E₄*E₅ | Eₐ |
| 3043 | E₁ₐE₂E₃E₄ | AlkLEᵥᵢᵢ | E₄*E₅ | Eₐ |
| 3044 | E_{1b}E₂E₃E₄ | AlkLEᵥᵢᵢ | E₄*E₅ | Eₐ |
| 3045 | E₁ₐE₂EᵢE₄ | AlkLEᵥᵢᵢ | E₄*E₅ | Eₐ |
| 3046 | E_{1b}E₂EᵢE₄ | AlkLEᵥᵢᵢ | E₄*E₅ | Eₐ |
| 3047 | E₁ₐE₂EᵢᵢE₄ | AlkLEᵥᵢᵢ | E₄*E₅ | Eₐ |
| 3048 | E_{1b}E₂EᵢᵢE₄ | AlkLEᵥᵢᵢ | E₄*E₅ | Eₐ |
| 3049 | E₁ₐE₂EᵢᵢE_{iib}E₄ | AlkLEᵥᵢᵢ | E₄*E₅ | Eₐ |
| 3050 | E_{1b}E₂EᵢᵢE_{iib}E₄ | AlkLEᵥᵢᵢ | E₄*E₅ | Eₐ |
| 3051 | E₁ₐE₂E₃EᵢE₄ | AlkLEᵥᵢᵢ | E₄*E₅ | Eₐ |
| 3052 | E_{1b}E₂E₃EᵢE₄ | AlkLEᵥᵢᵢ | E₄*E₅ | Eₐ |
| 3053 | E₁ₐE₂E₃EᵢᵢE₄ | AlkLEᵥᵢᵢ | E₄*E₅ | Eₐ |
| 3054 | E_{1b}E₂E₃EᵢᵢE₄ | AlkLEᵥᵢᵢ | E₄*E₅ | Eₐ |
| 3055 | E₁ₐE₂E₃EᵢᵢE_{iib}E₄ | AlkLEᵥᵢᵢ | E₄*E₅ | Eₐ |
| 3056 | E_{1b}E₂E₃EᵢᵢE_{iib}E₄ | AlkLEᵥᵢᵢ | E₄*E₅ | Eₐ |
| 3057 | E₁ₐE₂Eᵢᵢᵢ | AlkLEᵥᵢᵢ | E₄*E₅ | Eₐ |
| 3058 | E_{1b}E₂Eᵢᵢᵢ | AlkLEᵥᵢᵢ | E₄*E₅ | Eₐ |
| 3059 | E₁ₐE₂E₃Eᵢᵢᵢ | AlkLEᵥᵢᵢ | E₄*E₅ | Eₐ |
| 3060 | E_{1b}E₂E₃Eᵢᵢᵢ | AlkLEᵥᵢᵢ | E₄*E₅ | Eₐ |
| 3061 | E₁ₐE₂EᵢᵢᵢE₄ | AlkLEᵥᵢᵢ | E₄*E₅ | Eₐ |
| 3062 | E_{1b}E₂EᵢᵢᵢE₄ | AlkLEᵥᵢᵢ | E₄*E₅ | Eₐ |
| 3063 | E₁ₐE₂E₃EᵢᵢᵢE₄ | AlkLEᵥᵢᵢ | E₄*E₅ | Eₐ |
| 3064 | E_{1b}E₂E₃EᵢᵢᵢE₄ | AlkLEᵥᵢᵢ | E₄*E₅ | Eₐ |
| 3065 | E₁ₐE₂Eᵢᵥ | AlkLEᵥᵢᵢ | E₄*E₅ | Eₐ |
| 3066 | E_{1b}E₂Eᵢᵥ | AlkLEᵥᵢᵢ | E₄*E₅ | Eₐ |
| 3067 | E₁ₐE₂E₃Eᵢᵥ | AlkLEᵥᵢᵢ | E₄*E₅ | Eₐ |
| 3068 | E_{1b}E₂E₃Eᵢᵥ | AlkLEᵥᵢᵢ | E₄*E₅ | Eₐ |
| 3069 | E₁ₐE₂EᵢᵥE₄ | AlkLEᵥᵢᵢ | E₄*E₅ | Eₐ |
| 3070 | E_{1b}E₂EᵢᵥE₄ | AlkLEᵥᵢᵢ | E₄*E₅ | Eₐ |
| 3071 | E₁ₐE₂E₃EᵢᵥE₄ | AlkLEᵥᵢᵢ | E₄*E₅ | Eₐ |
| 3072 | E_{1b}E₂E₃EᵢᵥE₄ | AlkLEᵥᵢᵢ | E₄*E₅ | Eₐ |
| 3073 | E₁ₐE₂ | Eᵥᵢᵢ | | E_{b} |
| 3074 | E_{1b}E₂ | Eᵥᵢᵢ | | E_{b} |
| 3075 | E₁ₐE₂E₃ | Eᵥᵢᵢ | | E_{b} |
| 3076 | E_{1b}E₂E₃ | Eᵥᵢᵢ | | E_{b} |
| 3077 | E₁ₐE₂Eᵢ | Eᵥᵢᵢ | | E_{b} |
| 3078 | E_{1b}E₂Eᵢ | Eᵥᵢᵢ | | E_{b} |
| 3079 | E₁ₐE₂Eᵢᵢ | Eᵥᵢᵢ | | E_{b} |
| 3080 | E_{1b}E₂Eᵢᵢ | Eᵥᵢᵢ | | E_{b} |
| 3081 | E₁ₐE₂EᵢᵢE_{iib} | Eᵥᵢᵢ | | E_{b} |
| 3082 | E_{1b}E₂EᵢᵢE_{iib} | Eᵥᵢᵢ | | E_{b} |
| 3083 | E₁ₐE₂E₃Eᵢ | Eᵥᵢᵢ | | E_{b} |
| 3084 | E_{1b}E₂E₃Eᵢ | Eᵥᵢᵢ | | E_{b} |
| 3085 | E₁ₐE₂E₃Eᵢᵢ | Eᵥᵢᵢ | | E_{b} |
| 3086 | E_{1b}E₂E₃Eᵢᵢ | Eᵥᵢᵢ | | E_{b} |
| 3087 | E₁ₐE₂E₃EᵢᵢE_{iib} | Eᵥᵢᵢ | | E_{b} |
| 3088 | E_{1b}E₂E₃EᵢᵢE_{iib} | Eᵥᵢᵢ | | E_{b} |
| 3089 | E₁ₐE₂E₄ | Eᵥᵢᵢ | | E_{b} |
| 3090 | E_{1b}E₂E₄ | Eᵥᵢᵢ | | E_{b} |
| 3091 | E₁ₐE₂E₃E₄ | Eᵥᵢᵢ | | E_{b} |
| 3092 | E_{1b}E₂E₃E₄ | Eᵥᵢᵢ | | E_{b} |
| 3093 | E₁ₐE₂EᵢE₄ | Eᵥᵢᵢ | | E_{b} |
| 3094 | E_{1b}E₂EᵢE₄ | Eᵥᵢᵢ | | E_{b} |
| 3095 | E₁ₐE₂EᵢᵢE₄ | Eᵥᵢᵢ | | E_{b} |
| 3096 | E_{1b}E₂EᵢᵢE₄ | Eᵥᵢᵢ | | E_{b} |
| 3097 | E₁ₐE₂EᵢᵢE_{iib}E₄ | Eᵥᵢᵢ | | E_{b} |
| 3098 | E_{1b}E₂EᵢᵢE_{iib}E₄ | Eᵥᵢᵢ | | E_{b} |
| 3099 | E₁ₐE₂E₃EᵢE₄ | Eᵥᵢᵢ | | E_{b} |
| 3100 | E_{1b}E₂E₃EᵢE₄ | Eᵥᵢᵢ | | E_{b} |
| 3101 | E₁ₐE₂E₃EᵢᵢE₄ | Eᵥᵢᵢ | | E_{b} |
| 3102 | E_{1b}E₂E₃EᵢᵢE₄ | Eᵥᵢᵢ | | E_{b} |
| 3103 | E₁ₐE₂E₃EᵢᵢE_{iib}E₄ | Eᵥᵢᵢ | | E_{b} |
| 3104 | E_{1b}E₂E₃EᵢᵢE_{iib}E₄ | Eᵥᵢᵢ | | E_{b} |
| 3105 | E₁ₐE₂Eᵢᵢᵢ | Eᵥᵢᵢ | | E_{b} |
| 3106 | E_{1b}E₂Eᵢᵢᵢ | Eᵥᵢᵢ | | E_{b} |
| 3107 | E₁ₐE₂E₃Eᵢᵢᵢ | Eᵥᵢᵢ | | E_{b} |
| 3108 | E_{1b}E₂E₃Eᵢᵢᵢ | Eᵥᵢᵢ | | E_{b} |
| 3109 | E₁ₐE₂EᵢᵢᵢE₄ | Eᵥᵢᵢ | | E_{b} |
| 3110 | E_{1b}E₂EᵢᵢᵢE₄ | Eᵥᵢᵢ | | E_{b} |
| 3111 | E₁ₐE₂E₃EᵢᵢᵢE₄ | Eᵥᵢᵢ | | E_{b} |
| 3112 | E_{1b}E₂E₃EᵢᵢᵢE₄ | Eᵥᵢᵢ | | E_{b} |
| 3113 | E₁ₐE₂Eᵢᵥ | Eᵥᵢᵢ | | E_{b} |
| 3114 | E_{1b}E₂Eᵢᵥ | Eᵥᵢᵢ | | E_{b} |
| 3115 | E₁ₐE₂E₃Eᵢᵥ | Eᵥᵢᵢ | | E_{b} |
| 3116 | E_{1b}E₂E₃Eᵢᵥ | Eᵥᵢᵢ | | E_{b} |
| 3117 | E₁ₐE₂EᵢᵥE₄ | Eᵥᵢᵢ | | E_{b} |
| 3118 | E_{1b}E₂EᵢᵥE₄ | Eᵥᵢᵢ | | E_{b} |
| 3119 | E₁ₐE₂E₃EᵢᵥE₄ | Eᵥᵢᵢ | | E_{b} |
| 3120 | E_{1b}E₂E₃EᵢᵥE₄ | Eᵥᵢᵢ | | E_{b} |
| 3121 | E₁ₐE₂ | Eᵥᵢᵢ | E₄* | E_{b} |
| 3122 | E_{1b}E₂ | Eᵥᵢᵢ | E₄* | E_{b} |
| 3123 | E₁ₐE₂E₃ | Eᵥᵢᵢ | E₄* | E_{b} |
| 3124 | E_{1b}E₂E₃ | Eᵥᵢᵢ | E₄* | E_{b} |
| 3125 | E₁ₐE₂Eᵢ | Eᵥᵢᵢ | E₄* | E_{b} |
| 3126 | E_{1b}E₂Eᵢ | Eᵥᵢᵢ | E₄* | E_{b} |
| 3127 | E₁ₐE₂Eᵢᵢ | Eᵥᵢᵢ | E₄* | E_{b} |
| 3128 | E_{1b}E₂Eᵢᵢ | Eᵥᵢᵢ | E₄* | E_{b} |
| 3129 | E₁ₐE₂EᵢᵢE_{iib} | Eᵥᵢᵢ | E₄* | E_{b} |
| 3130 | E_{1b}E₂EᵢᵢE_{iib} | Eᵥᵢᵢ | E₄* | E_{b} |
| 3131 | E₁ₐE₂E₃Eᵢ | Eᵥᵢᵢ | E₄* | E_{b} |
| 3132 | E_{1b}E₂E₃Eᵢ | Eᵥᵢᵢ | E₄* | E_{b} |
| 3133 | E₁ₐE₂E₃Eᵢᵢ | Eᵥᵢᵢ | E₄* | E_{b} |
| 3134 | E_{1b}E₂E₃Eᵢᵢ | Eᵥᵢᵢ | E₄* | E_{b} |
| 3135 | E₁ₐE₂E₃EᵢᵢE_{iib} | Eᵥᵢᵢ | E₄* | E_{b} |
| 3136 | E_{1b}E₂E₃EᵢᵢE_{iib} | Eᵥᵢᵢ | E₄* | E_{b} |
| 3137 | E₁ₐE₂E₄ | Eᵥᵢᵢ | E₄* | E_{b} |
| 3138 | E_{1b}E₂E₄ | Eᵥᵢᵢ | E₄* | E_{b} |
| 3139 | E₁ₐE₂E₃E₄ | Eᵥᵢᵢ | E₄* | E_{b} |
| 3140 | E_{1b}E₂E₃E₄ | Eᵥᵢᵢ | E₄* | E_{b} |
| 3141 | E₁ₐE₂EᵢE₄ | Eᵥᵢᵢ | E₄* | E_{b} |
| 3142 | E_{1b}E₂EᵢE₄ | Eᵥᵢᵢ | E₄* | E_{b} |
| 3143 | E₁ₐE₂EᵢᵢE₄ | Eᵥᵢᵢ | E₄* | E_{b} |
| 3144 | E_{1b}E₂EᵢᵢE₄ | Eᵥᵢᵢ | E₄* | E_{b} |
| 3145 | E₁ₐE₂EᵢᵢE_{iib}E₄ | Eᵥᵢᵢ | E₄* | E_{b} |
| 3146 | E_{1b}E₂EᵢᵢE_{iib}E₄ | Eᵥᵢᵢ | E₄* | E_{b} |
| 3147 | E₁ₐE₂E₃EᵢE₄ | Eᵥᵢᵢ | E₄* | E_{b} |
| 3148 | E_{1b}E₂E₃EᵢE₄ | Eᵥᵢᵢ | E₄* | E_{b} |
| 3149 | E₁ₐE₂E₃EᵢᵢE₄ | Eᵥᵢᵢ | E₄* | E_{b} |
| 3150 | E_{1b}E₂E₃EᵢᵢE₄ | Eᵥᵢᵢ | E₄* | E_{b} |
| 3151 | E₁ₐE₂E₃EᵢᵢE_{iib}E₄ | Eᵥᵢᵢ | E₄* | E_{b} |
| 3152 | E_{1b}E₂E₃EᵢᵢE_{iib}E₄ | Eᵥᵢᵢ | E₄* | E_{b} |
| 3153 | E₁ₐE₂Eᵢᵢᵢ | Eᵥᵢᵢ | E₄* | E_{b} |
| 3154 | E_{1b}E₂Eᵢᵢᵢ | Eᵥᵢᵢ | E₄* | E_{b} |
| 3155 | E₁ₐE₂E₃Eᵢᵢᵢ | Eᵥᵢᵢ | E₄* | E_{b} |
| 3156 | E_{1b}E₂E₃Eᵢᵢᵢ | Eᵥᵢᵢ | E₄* | E_{b} |
| 3157 | E₁ₐE₂EᵢᵢᵢE₄ | Eᵥᵢᵢ | E₄* | E_{b} |
| 3158 | E_{1b}E₂EᵢᵢᵢE₄ | Eᵥᵢᵢ | E₄* | E_{b} |
| 3159 | E₁ₐE₂E₃EᵢᵢᵢE₄ | Eᵥᵢᵢ | E₄* | E_{b} |
| 3160 | E_{1b}E₂E₃EᵢᵢᵢE₄ | Eᵥᵢᵢ | E₄* | E_{b} |
| 3161 | E₁ₐE₂Eᵢᵥ | Eᵥᵢᵢ | E₄* | E_{b} |
| 3162 | E_{1b}E₂Eᵢᵥ | Eᵥᵢᵢ | E₄* | E_{b} |
| 3163 | E₁ₐE₂E₃Eᵢᵥ | Eᵥᵢᵢ | E₄* | E_{b} |
| 3164 | E_{1b}E₂E₃Eᵢᵥ | Eᵥᵢᵢ | E₄* | E_{b} |
| 3165 | E₁ₐE₂EᵢᵥE₄ | Eᵥᵢᵢ | E₄* | E_{b} |
| 3166 | E_{1b}E₂EᵢᵥE₄ | Eᵥᵢᵢ | E₄* | E_{b} |
| 3167 | E₁ₐE₂E₃EᵢᵥE₄ | Eᵥᵢᵢ | E₄* | E_{b} |
| 3168 | E_{1b}E₂E₃EᵢᵥE₄ | Eᵥᵢᵢ | E₄* | E_{b} |
| 3169 | E₁ₐE₂ | Eᵥᵢᵢ | E₅ | E_{b} |
| 3170 | E_{1b}E₂ | Eᵥᵢᵢ | E₅ | E_{b} |
| 3171 | E₁ₐE₂E₃ | Eᵥᵢᵢ | E₅ | E_{b} |
| 3172 | E_{1b}E₂E₃ | Eᵥᵢᵢ | E₅ | E_{b} |
| 3173 | E₁ₐE₂Eᵢ | Eᵥᵢᵢ | E₅ | E_{b} |
| 3174 | E_{1b}E₂Eᵢ | Eᵥᵢᵢ | E₅ | E_{b} |
| 3175 | E₁ₐE₂Eᵢᵢ | Eᵥᵢᵢ | E₅ | E_{b} |
| 3176 | E_{1b}E₂Eᵢᵢ | Eᵥᵢᵢ | E₅ | E_{b} |
| 3177 | E₁ₐE₂EᵢᵢE_{iib} | Eᵥᵢᵢ | E₅ | E_{b} |
| 3178 | E_{1b}E₂EᵢᵢE_{iib} | Eᵥᵢᵢ | E₅ | E_{b} |
| 3179 | E₁ₐE₂E₃Eᵢ | Eᵥᵢᵢ | E₅ | E_{b} |
| 3180 | E_{1b}E₂E₃Eᵢ | Eᵥᵢᵢ | E₅ | E_{b} |
| 3181 | E₁ₐE₂E₃Eᵢᵢ | Eᵥᵢᵢ | E₅ | E_{b} |
| 3182 | E_{1b}E₂E₃Eᵢᵢ | Eᵥᵢᵢ | E₅ | E_{b} |
| 3183 | E₁ₐE₂E₃EᵢᵢE_{iib} | Eᵥᵢᵢ | E₅ | E_{b} |
| 3184 | E_{1b}E₂E₃EᵢᵢE_{iib} | Eᵥᵢᵢ | E₅ | E_{b} |
| 3185 | E₁ₐE₂E₄ | Eᵥᵢᵢ | E₅ | E_{b} |
| 3186 | E_{1b}E₂E₄ | Eᵥᵢᵢ | E₅ | E_{b} |
| 3187 | E₁ₐE₂E₃E₄ | Eᵥᵢᵢ | E₅ | E_{b} |
| 3188 | E_{1b}E₂E₃E₄ | Eᵥᵢᵢ | E₅ | E_{b} |
| 3189 | E₁ₐE₂EᵢE₄ | Eᵥᵢᵢ | E₅ | E_{b} |
| 3190 | E_{1b}E₂EᵢE₄ | Eᵥᵢᵢ | E₅ | E_{b} |
| 3191 | E₁ₐE₂EᵢᵢE₄ | Eᵥᵢᵢ | E₅ | E_{b} |
| 3192 | E_{1b}E₂EᵢᵢE₄ | Eᵥᵢᵢ | E₅ | E_{b} |
| 3193 | E₁ₐE₂EᵢᵢE_{iib}E₄ | Eᵥᵢᵢ | E₅ | E_{b} |
| 3194 | E_{1b}E₂EᵢᵢE_{iib}E₄ | Eᵥᵢᵢ | E₅ | E_{b} |
| 3195 | E₁ₐE₂E₃EᵢE₄ | Eᵥᵢᵢ | E₅ | E_{b} |
| 3196 | E_{1b}E₂E₃EᵢE₄ | Eᵥᵢᵢ | E₅ | E_{b} |
| 3197 | E₁ₐE₂E₃EᵢᵢE₄ | Eᵥᵢᵢ | E₅ | E_{b} |
| 3198 | E_{1b}E₂E₃EᵢᵢE₄ | Eᵥᵢᵢ | E₅ | E_{b} |
| 3199 | E₁ₐE₂E₃EᵢᵢE_{iib}E₄ | Eᵥᵢᵢ | E₅ | E_{b} |
| 3200 | E_{1b}E₂E₃EᵢᵢE_{iib}E₄ | Eᵥᵢᵢ | E₅ | E_{b} |
| 3201 | E₁ₐE₂Eᵢᵢᵢ | Eᵥᵢᵢ | E₅ | E_{b} |
| 3202 | E_{1b}E₂Eᵢᵢᵢ | Eᵥᵢᵢ | E₅ | E_{b} |
| 3203 | E₁ₐE₂E₃Eᵢᵢᵢ | Eᵥᵢᵢ | E₅ | E_{b} |
| 3204 | E_{1b}E₂E₃Eᵢᵢᵢ | Eᵥᵢᵢ | E₅ | E_{b} |
| 3205 | E₁ₐE₂EᵢᵢᵢE₄ | Eᵥᵢᵢ | E₅ | E_{b} |
| 3206 | E_{1b}E₂EᵢᵢᵢE₄ | Eᵥᵢᵢ | E₅ | E_{b} |
| 3207 | E₁ₐE₂E₃EᵢᵢᵢE₄ | Eᵥᵢᵢ | E₅ | E_{b} |
| 3208 | E_{1b}E₂E₃EᵢᵢᵢE₄ | Eᵥᵢᵢ | E₅ | E_{b} |
| 3209 | E₁ₐE₂Eᵢᵥ | Eᵥᵢᵢ | E₅ | E_{b} |
| 3210 | E_{1b}E₂Eᵢᵥ | Eᵥᵢᵢ | E₅ | E_{b} |
| 3211 | E₁ₐE₂E₃Eᵢᵥ | Eᵥᵢᵢ | E₅ | E_{b} |
| 3212 | E_{1b}E₂E₃Eᵢᵥ | Eᵥᵢᵢ | E₅ | E_{b} |
| 3213 | E₁ₐE₂EᵢᵥE₄ | Eᵥᵢᵢ | E₅ | E_{b} |
| 3214 | E_{1b}E₂EᵢᵥE₄ | Eᵥᵢᵢ | E₅ | E_{b} |
| 3215 | E₁ₐE₂E₃EᵢᵥE₄ | Eᵥᵢᵢ | E₅ | E_{b} |
| 3216 | E_{1b}E₂E₃EᵢᵥE₄ | Eᵥᵢᵢ | E₅ | E_{b} |
| 3217 | E₁ₐE₂ | Eᵥᵢᵢ | E₄*E₅ | E_{b} |
| 3218 | E_{1b}E₂ | Eᵥᵢᵢ | E₄*E₅ | E_{b} |
| 3219 | E₁ₐE₂E₃ | Eᵥᵢᵢ | E₄*E₅ | E_{b} |
| 3220 | E_{1b}E₂E₃ | Eᵥᵢᵢ | E₄*E₅ | E_{b} |
| 3221 | E₁ₐE₂Eᵢ | Eᵥᵢᵢ | E₄*E₅ | E_{b} |
| 3222 | E_{1b}E₂Eᵢ | Eᵥᵢᵢ | E₄*E₅ | E_{b} |
| 3223 | E₁ₐE₂Eᵢᵢ | Eᵥᵢᵢ | E₄*E₅ | E_{b} |
| 3224 | E_{1b}E₂Eᵢᵢ | Eᵥᵢᵢ | E₄*E₅ | E_{b} |
| 3225 | E₁ₐE₂EᵢᵢE_{iib} | Eᵥᵢᵢ | E₄*E₅ | E_{b} |
| 3226 | E_{1b}E₂EᵢᵢE_{iib} | Eᵥᵢᵢ | E₄*E₅ | E_{b} |
| 3227 | E₁ₐE₂E₃Eᵢ | Eᵥᵢᵢ | E₄*E₅ | E_{b} |
| 3228 | E_{1b}E₂E₃Eᵢ | Eᵥᵢᵢ | E₄*E₅ | E_{b} |
| 3229 | E₁ₐE₂E₃Eᵢᵢ | Eᵥᵢᵢ | E₄*E₅ | E_{b} |
| 3230 | E_{1b}E₂E₃Eᵢᵢ | Eᵥᵢᵢ | E₄*E₅ | E_{b} |
| 3231 | E₁ₐE₂E₃EᵢᵢE_{iib} | Eᵥᵢᵢ | E₄*E₅ | E_{b} |
| 3232 | E_{1b}E₂E₃EᵢᵢE_{iib} | Eᵥᵢᵢ | E₄*E₅ | E_{b} |
| 3233 | E₁ₐE₂E₄ | Eᵥᵢᵢ | E₄*E₅ | E_{b} |
| 3234 | E_{1b}E₂E₄ | Eᵥᵢᵢ | E₄*E₅ | E_{b} |
| 3235 | E₁ₐE₂E₃E₄ | Eᵥᵢᵢ | E₄*E₅ | E_{b} |
| 3236 | E_{1b}E₂E₃E₄ | Eᵥᵢᵢ | E₄*E₅ | E_{b} |
| 3237 | E₁ₐE₂EᵢE₄ | Eᵥᵢᵢ | E₄*E₅ | E_{b} |
| 3238 | E_{1b}E₂EᵢE₄ | Eᵥᵢᵢ | E₄*E₅ | E_{b} |
| 3239 | E₁ₐE₂EᵢᵢE₄ | Eᵥᵢᵢ | E₄*E₅ | E_{b} |
| 3240 | E_{1b}E₂EᵢᵢE₄ | Eᵥᵢᵢ | E₄*E₅ | E_{b} |
| 3241 | E₁ₐE₂EᵢᵢE_{iib}E₄ | Eᵥᵢᵢ | E₄*E₅ | E_{b} |
| 3242 | E_{1b}E₂EᵢᵢE_{iib}E₄ | Eᵥᵢᵢ | E₄*E₅ | E_{b} |
| 3243 | E₁ₐE₂E₃EᵢE₄ | Eᵥᵢᵢ | E₄*E₅ | E_{b} |
| 3244 | E_{1b}E₂E₃EᵢE₄ | Eᵥᵢᵢ | E₄*E₅ | E_{b} |
| 3245 | E₁ₐE₂E₃EᵢᵢE₄ | Eᵥᵢᵢ | E₄*E₅ | E_{b} |
| 3246 | E_{1b}E₂E₃EᵢᵢE₄ | Eᵥᵢᵢ | E₄*E₅ | E_{b} |
| 3247 | E₁ₐE₂E₃EᵢᵢE_{iib}E₄ | Eᵥᵢᵢ | E₄*E₅ | E_{b} |
| 3248 | E_{1b}E₂E₃EᵢᵢE_{iib}E₄ | Eᵥᵢᵢ | E₄*E₅ | E_{b} |
| 3249 | E₁ₐE₂Eᵢᵢᵢ | Eᵥᵢᵢ | E₄*E₅ | E_{b} |
| 3250 | E_{1b}E₂Eᵢᵢᵢ | Eᵥᵢᵢ | E₄*E₅ | E_{b} |
| 3251 | E₁ₐE₂E₃Eᵢᵢᵢ | Eᵥᵢᵢ | E₄*E₅ | E_{b} |
| 3252 | E_{1b}E₂E₃Eᵢᵢᵢ | Eᵥᵢᵢ | E₄*E₅ | E_{b} |
| 3253 | E₁ₐE₂EᵢᵢᵢE₄ | Eᵥᵢᵢ | E₄*E₅ | E_{b} |
| 3254 | E_{1b}E₂EᵢᵢᵢE₄ | Eᵥᵢᵢ | E₄*E₅ | E_{b} |
| 3255 | E₁ₐE₂E₃EᵢᵢᵢE₄ | Eᵥᵢᵢ | E₄*E₅ | E_{b} |
| 3256 | E_{1b}E₂E₃EᵢᵢᵢE₄ | Eᵥᵢᵢ | E₄*E₅ | E_{b} |
| 3257 | E₁ₐE₂Eᵢᵥ | Eᵥᵢᵢ | E₄*E₅ | E_{b} |
| 3258 | E_{1b}E₂Eᵢᵥ | Eᵥᵢᵢ | E₄*E₅ | E_{b} |
| 3259 | E₁ₐE₂E₃Eᵢᵥ | Eᵥᵢᵢ | E₄*E₅ | E_{b} |
| 3260 | E_{1b}E₂E₃Eᵢᵥ | Eᵥᵢᵢ | E₄*E₅ | E_{b} |
| 3261 | E₁ₐE₂EᵢᵥE₄ | Eᵥᵢᵢ | E₄*E₅ | E_{b} |
| 3262 | E_{1b}E₂EᵢᵥE₄ | Eᵥᵢᵢ | E₄*E₅ | E_{b} |
| 3263 | E₁ₐE₂E₃EᵢᵥE₄ | Eᵥᵢᵢ | E₄*E₅ | E_{b} |
| 3264 | E_{1b}E₂E₃EᵢᵥE₄ | Eᵥᵢᵢ | E₄*E₅ | E_{b} |
| 3265 | E₁ₐE₂ | AlkLEᵥᵢᵢ | | E_{b} |
| 3266 | E_{1b}E₂ | AlkLEᵥᵢᵢ | | E_{b} |
| 3267 | E₁ₐE₂E₃ | AlkLEᵥᵢᵢ | | E_{b} |
| 3268 | E_{1b}E₂E₃ | AlkLEᵥᵢᵢ | | E_{b} |
| 3269 | E₁ₐE₂Eᵢ | AlkLEᵥᵢᵢ | | E_{b} |
| 3270 | E_{1b}E₂Eᵢ | AlkLEᵥᵢᵢ | | E_{b} |
| 3271 | E₁ₐE₂Eᵢᵢ | AlkLEᵥᵢᵢ | | E_{b} |
| 3272 | E_{1b}E₂Eᵢᵢ | AlkLEᵥᵢᵢ | | E_{b} |
| 3273 | E₁ₐE₂EᵢᵢE_{iib} | AlkLEᵥᵢᵢ | | E_{b} |
| 3274 | E_{1b}E₂EᵢᵢE_{iib} | AlkLEᵥᵢᵢ | | E_{b} |
| 3275 | E₁ₐE₂E₃Eᵢ | AlkLEᵥᵢᵢ | | E_{b} |
| 3276 | E_{1b}E₂E₃Eᵢ | AlkLEᵥᵢᵢ | | E_{b} |
| 3277 | E₁ₐE₂E₃Eᵢᵢ | AlkLEᵥᵢᵢ | | E_{b} |
| 3278 | E_{1b}E₂E₃Eᵢᵢ | AlkLEᵥᵢᵢ | | E_{b} |
| 3279 | E₁ₐE₂E₃EᵢᵢE_{iib} | AlkLEᵥᵢᵢ | | E_{b} |
| 3280 | E_{1b}E₂E₃EᵢᵢE_{iib} | AlkLEᵥᵢᵢ | | E_{b} |
| 3281 | E₁ₐE₂E₄ | AlkLEᵥᵢᵢ | | E_{b} |
| 3282 | E_{1b}E₂E₄ | AlkLEᵥᵢᵢ | | E_{b} |
| 3283 | E₁ₐE₂E₃E₄ | AlkLEᵥᵢᵢ | | E_{b} |
| 3284 | E_{1b}E₂E₃E₄ | AlkLEᵥᵢᵢ | | E_{b} |
| 3285 | E₁ₐE₂EᵢE₄ | AlkLEᵥᵢᵢ | | E_{b} |
| 3286 | E_{1b}E₂EᵢE₄ | AlkLEᵥᵢᵢ | | E_{b} |
| 3287 | E₁ₐE₂EᵢᵢE₄ | AlkLEᵥᵢᵢ | | E_{b} |
| 3288 | E_{1b}E₂EᵢᵢE₄ | AlkLEᵥᵢᵢ | | E_{b} |
| 3289 | E₁ₐE₂EᵢᵢE_{iib}E₄ | AlkLEᵥᵢᵢ | | E_{b} |
| 3290 | E_{1b}E₂EᵢᵢE_{iib}E₄ | AlkLEᵥᵢᵢ | | E_{b} |
| 3291 | E₁ₐE₂E₃EᵢE₄ | AlkLEᵥᵢᵢ | | E_{b} |
| 3292 | E_{1b}E₂E₃EᵢE₄ | AlkLEᵥᵢᵢ | | E_{b} |
| 3293 | E₁ₐE₂E₃EᵢᵢE₄ | AlkLEᵥᵢᵢ | | E_{b} |
| 3294 | E_{1b}E₂E₃EᵢᵢE₄ | AlkLEᵥᵢᵢ | | E_{b} |
| 3295 | E₁ₐE₂E₃EᵢᵢE_{iib}E₄ | AlkLEᵥᵢᵢ | | E_{b} |
| 3296 | E_{1b}E₂E₃EᵢᵢE_{iib}E₄ | AlkLEᵥᵢᵢ | | E_{b} |
| 3297 | E₁ₐE₂Eᵢᵢᵢ | AlkLEᵥᵢᵢ | | E_{b} |
| 3298 | E_{1b}E₂Eᵢᵢᵢ | AlkLEᵥᵢᵢ | | E_{b} |
| 3299 | E₁ₐE₂E₃Eᵢᵢᵢ | AlkLEᵥᵢᵢ | | E_{b} |
| 3300 | E_{1b}E₂E₃Eᵢᵢᵢ | AlkLEᵥᵢᵢ | | E_{b} |
| 3301 | E₁ₐE₂EᵢᵢᵢE₄ | AlkLEᵥᵢᵢ | | E_{b} |
| 3302 | E_{1b}E₂EᵢᵢᵢE₄ | AlkLEᵥᵢᵢ | | E_{b} |
| 3303 | E₁ₐE₂E₃EᵢᵢᵢE₄ | AlkLEᵥᵢᵢ | | E_{b} |
| 3304 | E_{1b}E₂E₃EᵢᵢᵢE₄ | AlkLEᵥᵢᵢ | | E_{b} |
| 3305 | E₁ₐE₂Eᵢᵥ | AlkLEᵥᵢᵢ | | E_{b} |
| 3306 | E_{1b}E₂Eᵢᵥ | AlkLEᵥᵢᵢ | | E_{b} |
| 3307 | E₁ₐE₂E₃Eᵢᵥ | AlkLEᵥᵢᵢ | | E_{b} |
| 3308 | E_{1b}E₂E₃Eᵢᵥ | AlkLEᵥᵢᵢ | | E_{b} |
| 3309 | E₁ₐE₂EᵢᵥE₄ | AlkLEᵥᵢᵢ | | E_{b} |
| 3310 | E_{1b}E₂EᵢᵥE₄ | AlkLEᵥᵢᵢ | | E_{b} |
| 3311 | E₁ₐE₂E₃EᵢᵥE₄ | AlkLEᵥᵢᵢ | | E_{b} |
| 3312 | E_{1b}E₂E₃EᵢᵥE₄ | AlkLEᵥᵢᵢ | | E_{b} |
| 3313 | E₁ₐE₂ | AlkLEᵥᵢᵢ | E₄* | E_{b} |
| 3314 | E_{1b}E₂ | AlkLEᵥᵢᵢ | E₄* | E_{b} |
| 3315 | E₁ₐE₂E₃ | AlkLEᵥᵢᵢ | E₄* | E_{b} |
| 3316 | E_{1b}E₂E₃ | AlkLEᵥᵢᵢ | E₄* | E_{b} |
| 3317 | E₁ₐE₂Eᵢ | AlkLEᵥᵢᵢ | E₄* | E_{b} |
| 3318 | E_{1b}E₂Eᵢ | AlkLEᵥᵢᵢ | E₄* | E_{b} |
| 3319 | E₁ₐE₂Eᵢᵢ | AlkLEᵥᵢᵢ | E₄* | E_{b} |
| 3320 | E_{1b}E₂Eᵢᵢ | AlkLEᵥᵢᵢ | E₄* | E_{b} |
| 3321 | E₁ₐE₂EᵢᵢE_{iib} | AlkLEᵥᵢᵢ | E₄* | E_{b} |
| 3322 | E_{1b}E₂EᵢᵢE_{iib} | AlkLEᵥᵢᵢ | E₄* | E_{b} |
| 3323 | E₁ₐE₂E₃Eᵢ | AlkLEᵥᵢᵢ | E₄* | E_{b} |
| 3324 | E_{1b}E₂E₃Eᵢ | AlkLEᵥᵢᵢ | E₄* | E_{b} |
| 3325 | E₁ₐE₂E₃Eᵢᵢ | AlkLEᵥᵢᵢ | E₄* | E_{b} |
| 3326 | E_{1b}E₂E₃Eᵢᵢ | AlkLEᵥᵢᵢ | E₄* | E_{b} |
| 3327 | E₁ₐE₂E₃EᵢᵢE_{iib} | AlkLEᵥᵢᵢ | E₄* | E_{b} |
| 3328 | E_{1b}E₂E₃EᵢᵢE_{iib} | AlkLEᵥᵢᵢ | E₄* | E_{b} |
| 3329 | E₁ₐE₂E₄ | AlkLEᵥᵢᵢ | E₄* | E_{b} |
| 3330 | E_{1b}E₂E₄ | AlkLEᵥᵢᵢ | E₄* | E_{b} |
| 3331 | E₁ₐE₂E₃E₄ | AlkLEᵥᵢᵢ | E₄* | E_{b} |
| 3332 | E_{1b}E₂E₃E₄ | AlkLEᵥᵢᵢ | E₄* | E_{b} |
| 3333 | E₁ₐE₂EᵢE₄ | AlkLEᵥᵢᵢ | E₄* | E_{b} |
| 3334 | E_{1b}E₂EᵢE₄ | AlkLEᵥᵢᵢ | E₄* | E_{b} |
| 3335 | E₁ₐE₂EᵢᵢE₄ | AlkLEᵥᵢᵢ | E₄* | E_{b} |
| 3336 | E_{1b}E₂EᵢᵢE₄ | AlkLEᵥᵢᵢ | E₄* | E_{b} |
| 3337 | E₁ₐE₂EᵢᵢE_{iib}E₄ | AlkLEᵥᵢᵢ | E₄* | E_{b} |
| 3338 | E_{1b}E₂EᵢᵢE_{iib}E₄ | AlkLEᵥᵢᵢ | E₄* | E_{b} |
| 3339 | E₁ₐE₂E₃EᵢE₄ | AlkLEᵥᵢᵢ | E₄* | E_{b} |
| 3340 | E_{1b}E₂E₃EᵢE₄ | AlkLEᵥᵢᵢ | E₄* | E_{b} |
| 3341 | E₁ₐE₂E₃EᵢᵢE₄ | AlkLEᵥᵢᵢ | E₄* | E_{b} |
| 3342 | E_{1b}E₂E₃EᵢᵢE₄ | AlkLEᵥᵢᵢ | E₄* | E_{b} |
| 3343 | E₁ₐE₂E₃EᵢᵢE_{iib}E₄ | AlkLEᵥᵢᵢ | E₄* | E_{b} |
| 3344 | E_{1b}E₂E₃EᵢᵢE_{iib}E₄ | AlkLEᵥᵢᵢ | E₄* | E_{b} |
| 3345 | E₁ₐE₂Eᵢᵢᵢ | AlkLEᵥᵢᵢ | E₄* | E_{b} |
| 3346 | E_{1b}E₂Eᵢᵢᵢ | AlkLEᵥᵢᵢ | E₄* | E_{b} |
| 3347 | E₁ₐE₂E₃Eᵢᵢᵢ | AlkLEᵥᵢᵢ | E₄* | E_{b} |
| 3348 | E_{1b}E₂E₃Eᵢᵢᵢ | AlkLEᵥᵢᵢ | E₄* | E_{b} |
| 3349 | E₁ₐE₂EᵢᵢᵢE₄ | AlkLEᵥᵢᵢ | E₄* | E_{b} |
| 3350 | E_{1b}E₂EᵢᵢᵢE₄ | AlkLEᵥᵢᵢ | E₄* | E_{b} |
| 3351 | E₁ₐE₂E₃EᵢᵢᵢE₄ | AlkLEᵥᵢᵢ | E₄* | E_{b} |
| 3352 | E_{1b}E₂E₃EᵢᵢᵢE₄ | AlkLEᵥᵢᵢ | E₄* | E_{b} |
| 3353 | E₁ₐE₂Eᵢᵥ | AlkLEᵥᵢᵢ | E₄* | E_{b} |
| 3354 | E_{1b}E₂Eᵢᵥ | AlkLEᵥᵢᵢ | E₄* | E_{b} |
| 3355 | E₁ₐE₂E₃Eᵢᵥ | AlkLEᵥᵢᵢ | E₄* | E_{b} |
| 3356 | E_{1b}E₂E₃Eᵢᵥ | AlkLEᵥᵢᵢ | E₄* | E_{b} |
| 3357 | E₁ₐE₂EᵢᵥE₄ | AlkLEᵥᵢᵢ | E₄* | E_{b} |
| 3358 | E_{1b}E₂EᵢᵥE₄ | AlkLEᵥᵢᵢ | E₄* | E_{b} |
| 3359 | E₁ₐE₂E₃EᵢᵥE₄ | AlkLEᵥᵢᵢ | E₄* | E_{b} |
| 3360 | E_{1b}E₂E₃EᵢᵥE₄ | AlkLEᵥᵢᵢ | E₄* | E_{b} |
| 3361 | E₁ₐE₂ | AlkLEᵥᵢᵢ | E₅ | E_{b} |
| 3362 | E_{1b}E₂ | AlkLEᵥᵢᵢ | E₅ | E_{b} |
| 3363 | E₁ₐE₂E₃ | AlkLEᵥᵢᵢ | E₅ | E_{b} |
| 3364 | E_{1b}E₂E₃ | AlkLEᵥᵢᵢ | E₅ | E_{b} |
| 3365 | E₁ₐE₂Eᵢ | AlkLEᵥᵢᵢ | E₅ | E_{b} |
| 3366 | E_{1b}E₂Eᵢ | AlkLEᵥᵢᵢ | E₅ | E_{b} |
| 3367 | E₁ₐE₂Eᵢᵢ | AlkLEᵥᵢᵢ | E₅ | E_{b} |
| 3368 | E_{1b}E₂Eᵢᵢ | AlkLEᵥᵢᵢ | E₅ | E_{b} |
| 3369 | E₁ₐE₂EᵢᵢE_{iib} | AlkLEᵥᵢᵢ | E₅ | E_{b} |
| 3370 | E_{1b}E₂EᵢᵢE_{iib} | AlkLEᵥᵢᵢ | E₅ | E_{b} |
| 3371 | E₁ₐE₂E₃Eᵢ | AlkLEᵥᵢᵢ | E₅ | E_{b} |
| 3372 | E_{1b}E₂E₃Eᵢ | AlkLEᵥᵢᵢ | E₅ | E_{b} |
| 3373 | E₁ₐE₂E₃Eᵢᵢ | AlkLEᵥᵢᵢ | E₅ | E_{b} |
| 3374 | E_{1b}E₂E₃Eᵢᵢ | AlkLEᵥᵢᵢ | E₅ | E_{b} |
| 3375 | E₁ₐE₂E₃EᵢᵢE_{iib} | AlkLEᵥᵢᵢ | E₅ | E_{b} |
| 3376 | E_{1b}E₂E₃EᵢᵢE_{iib} | AlkLEᵥᵢᵢ | E₅ | E_{b} |
| 3377 | E₁ₐE₂E₄ | AlkLEᵥᵢᵢ | E₅ | E_{b} |
| 3378 | E_{1b}E₂E₄ | AlkLEᵥᵢᵢ | E₅ | E_{b} |
| 3379 | E₁ₐE₂E₃E₄ | AlkLEᵥᵢᵢ | E₅ | E_{b} |
| 3380 | E_{1b}E₂E₃E₄ | AlkLEᵥᵢᵢ | E₅ | E_{b} |
| 3381 | E₁ₐE₂EᵢE₄ | AlkLEᵥᵢᵢ | E₅ | E_{b} |
| 3382 | E_{1b}E₂EᵢE₄ | AlkLEᵥᵢᵢ | E₅ | E_{b} |
| 3383 | E₁ₐE₂EᵢᵢE₄ | AlkLEᵥᵢᵢ | E₅ | E_{b} |
| 3384 | E_{1b}E₂EᵢᵢE₄ | AlkLEᵥᵢᵢ | E₅ | E_{b} |
| 3385 | E₁ₐE₂EᵢᵢE_{iib}E₄ | AlkLEᵥᵢᵢ | E₅ | E_{b} |
| 3386 | E_{1b}E₂EᵢᵢE_{iib}E₄ | AlkLEᵥᵢᵢ | E₅ | E_{b} |
| 3387 | E₁ₐE₂E₃EᵢE₄ | AlkLEᵥᵢᵢ | E₅ | E_{b} |
| 3388 | E_{1b}E₂E₃EᵢE₄ | AlkLEᵥᵢᵢ | E₅ | E_{b} |
| 3389 | E₁ₐE₂E₃EᵢᵢE₄ | AlkLEᵥᵢᵢ | E₅ | E_{b} |
| 3390 | E_{1b}E₂E₃EᵢᵢE₄ | AlkLEᵥᵢᵢ | E₅ | E_{b} |
| 3391 | E₁ₐE₂E₃EᵢᵢE_{iib}E₄ | AlkLEᵥᵢᵢ | E₅ | E_{b} |
| 3392 | E_{1b}E₂E₃EᵢᵢE_{iib}E₄ | AlkLEᵥᵢᵢ | E₅ | E_{b} |
| 3393 | E₁ₐE₂Eᵢᵢᵢ | AlkLEᵥᵢᵢ | E₅ | E_{b} |
| 3394 | E_{1b}E₂Eᵢᵢᵢ | AlkLEᵥᵢᵢ | E₅ | E_{b} |
| 3395 | E₁ₐE₂E₃Eᵢᵢᵢ | AlkLEᵥᵢᵢ | E₅ | E_{b} |
| 3396 | E_{1b}E₂E₃Eᵢᵢᵢ | AlkLEᵥᵢᵢ | E₅ | E_{b} |
| 3397 | E₁ₐE₂EᵢᵢᵢE₄ | AlkLEᵥᵢᵢ | E₅ | E_{b} |
| 3398 | E_{1b}E₂EᵢᵢᵢE₄ | AlkLEᵥᵢᵢ | E₅ | E_{b} |
| 3399 | E₁ₐE₂E₃EᵢᵢᵢE₄ | AlkLEᵥᵢᵢ | E₅ | E_{b} |
| 3400 | E_{1b}E₂E₃EᵢᵢᵢE₄ | AlkLEᵥᵢᵢ | E₅ | E_{b} |
| 3401 | E₁ₐE₂Eᵢᵥ | AlkLEᵥᵢᵢ | E₅ | E_{b} |
| 3402 | E_{1b}E₂Eᵢᵥ | AlkLEᵥᵢᵢ | E₅ | E_{b} |
| 3403 | E₁ₐE₂E₃Eᵢᵥ | AlkLEᵥᵢᵢ | E₅ | E_{b} |
| 3404 | E_{1b}E₂E₃Eᵢᵥ | AlkLEᵥᵢᵢ | E₅ | E_{b} |
| 3405 | E₁ₐE₂EᵢᵥE₄ | AlkLEᵥᵢᵢ | E₅ | E_{b} |
| 3406 | E_{1b}E₂EᵢᵥE₄ | AlkLEᵥᵢᵢ | E₅ | E_{b} |
| 3407 | E₁ₐE₂E₃EᵢᵥE₄ | AlkLEᵥᵢᵢ | E₅ | E_{b} |
| 3408 | E_{1b}E₂E₃EᵢᵥE₄ | AlkLEᵥᵢᵢ | E₅ | E_{b} |
| 3409 | E₁ₐE₂ | AlkLEᵥᵢᵢ | E₄*E₅ | E_{b} |
| 3410 | E_{1b}E₂ | AlkLEᵥᵢᵢ | E₄*E₅ | E_{b} |
| 3411 | E₁ₐE₂E₃ | AlkLEᵥᵢᵢ | E₄*E₅ | E_{b} |
| 3412 | E_{1b}E₂E₃ | AlkLEᵥᵢᵢ | E₄*E₅ | E_{b} |
| 3413 | E₁ₐE₂Eᵢ | AlkLEᵥᵢᵢ | E₄*E₅ | E_{b} |
| 3414 | E_{1b}E₂Eᵢ | AlkLEᵥᵢᵢ | E₄*E₅ | E_{b} |
| 3415 | E₁ₐE₂Eᵢᵢ | AlkLEᵥᵢᵢ | E₄*E₅ | E_{b} |
| 3416 | E_{1b}E₂Eᵢᵢ | AlkLEᵥᵢᵢ | E₄*E₅ | E_{b} |
| 3417 | E₁ₐE₂EᵢᵢE_{iib} | AlkLEᵥᵢᵢ | E₄*E₅ | E_{b} |
| 3418 | E_{1b}E₂EᵢᵢE_{iib} | AlkLEᵥᵢᵢ | E₄*E₅ | E_{b} |
| 3419 | E₁ₐE₂E₃Eᵢ | AlkLEᵥᵢᵢ | E₄*E₅ | E_{b} |
| 3420 | E_{1b}E₂E₃Eᵢ | AlkLEᵥᵢᵢ | E₄*E₅ | E_{b} |
| 3421 | E₁ₐE₂E₃Eᵢᵢ | AlkLEᵥᵢᵢ | E₄*E₅ | E_{b} |
| 3422 | E_{1b}E₂E₃Eᵢᵢ | AlkLEᵥᵢᵢ | E₄*E₅ | E_{b} |
| 3423 | E₁ₐE₂E₃EᵢᵢE_{iib} | AlkLEᵥᵢᵢ | E₄*E₅ | E_{b} |
| 3424 | E_{1b}E₂E₃EᵢᵢE_{iib} | AlkLEᵥᵢᵢ | E₄*E₅ | E_{b} |
| 3425 | E₁ₐE₂E₄ | AlkLEᵥᵢᵢ | E₄*E₅ | E_{b} |
| 3426 | E_{1b}E₂E₄ | AlkLEᵥᵢᵢ | E₄*E₅ | E_{b} |
| 3427 | E₁ₐE₂E₃E₄ | AlkLEᵥᵢᵢ | E₄*E₅ | E_{b} |
| 3428 | E_{1b}E₂E₃E₄ | AlkLEᵥᵢᵢ | E₄*E₅ | E_{b} |
| 3429 | E₁ₐE₂EᵢE₄ | AlkLEᵥᵢᵢ | E₄*E₅ | E_{b} |
| 3430 | E_{1b}E₂EᵢE₄ | AlkLEᵥᵢᵢ | E₄*E₅ | E_{b} |
| 3431 | E₁ₐE₂EᵢᵢE₄ | AlkLEᵥᵢᵢ | E₄*E₅ | E_{b} |
| 3432 | E_{1b}E₂EᵢᵢE₄ | AlkLEᵥᵢᵢ | E₄*E₅ | E_{b} |
| 3433 | E₁ₐE₂EᵢᵢE_{iib}E₄ | AlkLEᵥᵢᵢ | E₄*E₅ | E_{b} |
| 3434 | E_{1b}E₂EᵢᵢE_{iib}E₄ | AlkLEᵥᵢᵢ | E₄*E₅ | E_{b} |
| 3435 | E₁ₐE₂E₃EᵢE₄ | AlkLEᵥᵢᵢ | E₄*E₅ | E_{b} |
| 3436 | E_{1b}E₂E₃EᵢE₄ | AlkLEᵥᵢᵢ | E₄*E₅ | E_{b} |
| 3437 | E₁ₐE₂E₃EᵢᵢE₄ | AlkLEᵥᵢᵢ | E₄*E₅ | E_{b} |
| 3438 | E_{1b}E₂E₃EᵢᵢE₄ | AlkLEᵥᵢᵢ | E₄*E₅ | E_{b} |
| 3439 | E₁ₐE₂E₃EᵢᵢE_{iib}E₄ | AlkLEᵥᵢᵢ | E₄*E₅ | E_{b} |
| 3440 | E_{1b}E₂E₃EᵢᵢE_{iib}E₄ | AlkLEᵥᵢᵢ | E₄*E₅ | E_{b} |
| 3441 | E₁ₐE₂Eᵢᵢᵢ | AlkLEᵥᵢᵢ | E₄*E₅ | E_{b} |
| 3442 | E_{1b}E₂Eᵢᵢᵢ | AlkLEᵥᵢᵢ | E₄*E₅ | E_{b} |
| 3443 | E₁ₐE₂E₃Eᵢᵢᵢ | AlkLEᵥᵢᵢ | E₄*E₅ | E_{b} |
| 3444 | E_{1b}E₂E₃Eᵢᵢᵢ | AlkLEᵥᵢᵢ | E₄*E₅ | E_{b} |
| 3445 | E₁ₐE₂EᵢᵢᵢE₄ | AlkLEᵥᵢᵢ | E₄*E₅ | E_{b} |
| 3446 | E_{1b}E₂EᵢᵢᵢE₄ | AlkLEᵥᵢᵢ | E₄*E₅ | E_{b} |
| 3447 | E₁ₐE₂E₃EᵢᵢᵢE₄ | AlkLEᵥᵢᵢ | E₄*E₅ | E_{b} |
| 3448 | E_{1b}E₂E₃EᵢᵢᵢE₄ | AlkLEᵥᵢᵢ | E₄*E₅ | E_{b} |
| 3449 | E₁ₐE₂Eᵢᵥ | AlkLEᵥᵢᵢ | E₄*E₅ | E_{b} |
| 3450 | E_{1b}E₂Eᵢᵥ | AlkLEᵥᵢᵢ | E₄*E₅ | E_{b} |
| 3451 | E₁ₐE₂E₃Eᵢᵥ | AlkLEᵥᵢᵢ | E₄*E₅ | E_{b} |
| 3452 | E_{1b}E₂E₃Eᵢᵥ | AlkLEᵥᵢᵢ | E₄*E₅ | E_{b} |
| 3453 | E₁ₐE₂EᵢᵥE₄ | AlkLEᵥᵢᵢ | E₄*E₅ | E_{b} |
| 3454 | E_{1b}E₂EᵢᵥE₄ | AlkLEᵥᵢᵢ | E₄*E₅ | E_{b} |
| 3455 | E₁ₐE₂E₃EᵢᵥE₄ | AlkLEᵥᵢᵢ | E₄*E₅ | E_{b} |
| 3456 | E_{1b}E₂E₃EᵢᵥE₄ | AlkLEᵥᵢᵢ | E₄*E₅ | E_{b} |
| 3457 | E₁ₐE₂ | Eᵥᵢᵢ | | E_{d} |
| 3458 | E_{1b}E₂ | Eᵥᵢᵢ | | E_{d} |
| 3459 | E₁ₐE₂E₃ | Eᵥᵢᵢ | | E_{d} |
| 3460 | E_{1b}E₂E₃ | Eᵥᵢᵢ | | E_{d} |
| 3461 | E₁ₐE₂Eᵢ | Eᵥᵢᵢ | | E_{d} |
| 3462 | E_{1b}E₂Eᵢ | Eᵥᵢᵢ | | E_{d} |
| 3463 | E₁ₐE₂Eᵢᵢ | Eᵥᵢᵢ | | E_{d} |
| 3464 | E_{1b}E₂Eᵢᵢ | Eᵥᵢᵢ | | E_{d} |
| 3465 | E₁ₐE₂EᵢᵢE_{iib} | Eᵥᵢᵢ | | E_{d} |
| 3466 | E_{1b}E₂EᵢᵢE_{iib} | Eᵥᵢᵢ | | E_{d} |
| 3467 | E₁ₐE₂E₃Eᵢ | Eᵥᵢᵢ | | E_{d} |
| 3468 | E_{1b}E₂E₃Eᵢ | Eᵥᵢᵢ | | E_{d} |
| 3469 | E₁ₐE₂E₃Eᵢᵢ | Eᵥᵢᵢ | | E_{d} |
| 3470 | E_{1b}E₂E₃Eᵢᵢ | Eᵥᵢᵢ | | E_{d} |
| 3471 | E₁ₐE₂E₃EᵢᵢE_{iib} | Eᵥᵢᵢ | | E_{d} |
| 3472 | E_{1b}E₂E₃EᵢᵢE_{iib} | Eᵥᵢᵢ | | E_{d} |
| 3473 | E₁ₐE₂E₄ | Eᵥᵢᵢ | | E_{d} |
| 3474 | E_{1b}E₂E₄ | Eᵥᵢᵢ | | E_{d} |
| 3475 | E₁ₐE₂E₃E₄ | Eᵥᵢᵢ | | E_{d} |
| 3476 | E_{1b}E₂E₃E₄ | Eᵥᵢᵢ | | E_{d} |
| 3477 | E₁ₐE₂EᵢE₄ | Eᵥᵢᵢ | | E_{d} |
| 3478 | E_{1b}E₂EᵢE₄ | Eᵥᵢᵢ | | E_{d} |
| 3479 | E₁ₐE₂EᵢᵢE₄ | Eᵥᵢᵢ | | E_{d} |
| 3480 | E_{1b}E₂EᵢᵢE₄ | Eᵥᵢᵢ | | E_{d} |
| 3481 | E₁ₐE₂EᵢᵢE_{iib}E₄ | Eᵥᵢᵢ | | E_{d} |
| 3482 | E_{1b}E₂EᵢᵢE_{iib}E₄ | Eᵥᵢᵢ | | E_{d} |
| 3483 | E₁ₐE₂E₃EᵢE₄ | Eᵥᵢᵢ | | E_{d} |
| 3484 | E_{1b}E₂E₃EᵢE₄ | Eᵥᵢᵢ | | E_{d} |
| 3485 | E₁ₐE₂E₃EᵢᵢE₄ | Eᵥᵢᵢ | | E_{d} |
| 3486 | E_{1b}E₂E₃EᵢᵢE₄ | Eᵥᵢᵢ | | E_{d} |
| 3487 | E₁ₐE₂E₃EᵢᵢE_{iib}E₄ | Eᵥᵢᵢ | | E_{d} |
| 3488 | E_{1b}E₂E₃EᵢᵢE_{iib}E₄ | Eᵥᵢᵢ | | E_{d} |
| 3489 | E₁ₐE₂Eᵢᵢᵢ | Eᵥᵢᵢ | | E_{d} |
| 3490 | E_{1b}E₂Eᵢᵢᵢ | Eᵥᵢᵢ | | E_{d} |
| 3491 | E₁ₐE₂E₃Eᵢᵢᵢ | Eᵥᵢᵢ | | E_{d} |
| 3492 | E_{1b}E₂E₃Eᵢᵢᵢ | Eᵥᵢᵢ | | E_{d} |
| 3493 | E₁ₐE₂EᵢᵢᵢE₄ | Eᵥᵢᵢ | | E_{d} |
| 3494 | E_{1b}E₂EᵢᵢᵢE₄ | Eᵥᵢᵢ | | E_{d} |
| 3495 | E₁ₐE₂E₃EᵢᵢᵢE₄ | Eᵥᵢᵢ | | E_{d} |
| 3496 | E_{1b}E₂E₃EᵢᵢᵢE₄ | Eᵥᵢᵢ | | E_{d} |
| 3497 | E₁ₐE₂Eᵢᵥ | Eᵥᵢᵢ | | E_{d} |
| 3498 | E_{1b}E₂Eᵢᵥ | Eᵥᵢᵢ | | E_{d} |
| 3499 | E₁ₐE₂E₃Eᵢᵥ | Eᵥᵢᵢ | | E_{d} |
| 3500 | E_{1b}E₂E₃Eᵢᵥ | Eᵥᵢᵢ | | E_{d} |
| 3501 | E₁ₐE₂EᵢᵥE₄ | Eᵥᵢᵢ | | E_{d} |
| 3502 | E_{1b}E₂EᵢᵥE₄ | Eᵥᵢᵢ | | E_{d} |
| 3503 | E₁ₐE₂E₃EᵢᵥE₄ | Eᵥᵢᵢ | | E_{d} |
| 3504 | E_{1b}E₂E₃EᵢᵥE₄ | Eᵥᵢᵢ | | E_{d} |
| 3505 | E₁ₐE₂ | Eᵥᵢᵢ | E₄* | E_{d} |
| 3506 | E_{1b}E₂ | Eᵥᵢᵢ | E₄* | E_{d} |
| 3507 | E₁ₐE₂E₃ | Eᵥᵢᵢ | E₄* | E_{d} |
| 3508 | E_{1b}E₂E₃ | Eᵥᵢᵢ | E₄* | E_{d} |
| 3509 | E₁ₐE₂Eᵢ | Eᵥᵢᵢ | E₄* | E_{d} |
| 3510 | E_{1b}E₂Eᵢ | Eᵥᵢᵢ | E₄* | E_{d} |
| 3511 | E₁ₐE₂Eᵢᵢ | Eᵥᵢᵢ | E₄* | E_{d} |
| 3512 | E_{1b}E₂Eᵢᵢ | Eᵥᵢᵢ | E₄* | E_{d} |
| 3513 | E₁ₐE₂EᵢᵢE_{iib} | Eᵥᵢᵢ | E₄* | E_{d} |
| 3514 | E_{1b}E₂EᵢᵢE_{iib} | Eᵥᵢᵢ | E₄* | E_{d} |
| 3515 | E₁ₐE₂E₃Eᵢ | Eᵥᵢᵢ | E₄* | E_{d} |
| 3516 | E_{1b}E₂E₃Eᵢ | Eᵥᵢᵢ | E₄* | E_{d} |
| 3517 | E₁ₐE₂E₃Eᵢᵢ | Eᵥᵢᵢ | E₄* | E_{d} |
| 3518 | E_{1b}E₂E₃Eᵢᵢ | Eᵥᵢᵢ | E₄* | E_{d} |
| 3519 | E₁ₐE₂E₃EᵢᵢE_{iib} | Eᵥᵢᵢ | E₄* | E_{d} |
| 3520 | E_{1b}E₂E₃EᵢᵢE_{iib} | Eᵥᵢᵢ | E₄* | E_{d} |
| 3521 | E₁ₐE₂E₄ | Eᵥᵢᵢ | E₄* | E_{d} |
| 3522 | E_{1b}E₂E₄ | Eᵥᵢᵢ | E₄* | E_{d} |
| 3523 | E₁ₐE₂E₃E₄ | Eᵥᵢᵢ | E₄* | E_{d} |
| 3524 | E_{1b}E₂E₃E₄ | Eᵥᵢᵢ | E₄* | E_{d} |
| 3525 | E₁ₐE₂EᵢE₄ | Eᵥᵢᵢ | E₄* | E_{d} |
| 3526 | E_{1b}E₂EᵢE₄ | Eᵥᵢᵢ | E₄* | E_{d} |
| 3527 | E₁ₐE₂EᵢᵢE₄ | Eᵥᵢᵢ | E₄* | E_{d} |
| 3528 | E_{1b}E₂EᵢᵢE₄ | Eᵥᵢᵢ | E₄* | E_{d} |
| 3529 | E₁ₐE₂EᵢᵢE_{iib}E₄ | Eᵥᵢᵢ | E₄* | E_{d} |
| 3530 | E_{1b}E₂EᵢᵢE_{iib}E₄ | Eᵥᵢᵢ | E₄* | E_{d} |
| 3531 | E₁ₐE₂E₃EᵢE₄ | Eᵥᵢᵢ | E₄* | E_{d} |
| 3532 | E_{1b}E₂E₃EᵢE₄ | Eᵥᵢᵢ | E₄* | E_{d} |
| 3533 | E₁ₐE₂E₃EᵢᵢE₄ | Eᵥᵢᵢ | E₄* | E_{d} |
| 3534 | E_{1b}E₂E₃EᵢᵢE₄ | Eᵥᵢᵢ | E₄* | E_{d} |
| 3535 | E₁ₐE₂E₃EᵢᵢE_{iib}E₄ | Eᵥᵢᵢ | E₄* | E_{d} |
| 3536 | E_{1b}E₂E₃EᵢᵢE_{iib}E₄ | Eᵥᵢᵢ | E₄* | E_{d} |
| 3537 | E₁ₐE₂Eᵢᵢᵢ | Eᵥᵢᵢ | E₄* | E_{d} |
| 3538 | E_{1b}E₂Eᵢᵢᵢ | Eᵥᵢᵢ | E₄* | E_{d} |
| 3539 | E₁ₐE₂E₃Eᵢᵢᵢ | Eᵥᵢᵢ | E₄* | E_{d} |
| 3540 | E_{1b}E₂E₃Eᵢᵢᵢ | Eᵥᵢᵢ | E₄* | E_{d} |
| 3541 | E₁ₐE₂EᵢᵢᵢE₄ | Eᵥᵢᵢ | E₄* | E_{d} |
| 3542 | E_{1b}E₂EᵢᵢᵢE₄ | Eᵥᵢᵢ | E₄* | E_{d} |
| 3543 | E₁ₐE₂E₃EᵢᵢᵢE₄ | Eᵥᵢᵢ | E₄* | E_{d} |
| 3544 | E_{1b}E₂E₃EᵢᵢᵢE₄ | Eᵥᵢᵢ | E₄* | E_{d} |
| 3545 | E₁ₐE₂Eᵢᵥ | Eᵥᵢᵢ | E₄* | E_{d} |
| 3546 | E_{1b}E₂Eᵢᵥ | Eᵥᵢᵢ | E₄* | E_{d} |
| 3547 | E₁ₐE₂E₃Eᵢᵥ | Eᵥᵢᵢ | E₄* | E_{d} |
| 3548 | E_{1b}E₂E₃Eᵢᵥ | Eᵥᵢᵢ | E₄* | E_{d} |
| 3549 | E₁ₐE₂EᵢᵥE₄ | Eᵥᵢᵢ | E₄* | E_{d} |
| 3550 | E_{1b}E₂EᵢᵥE₄ | Eᵥᵢᵢ | E₄* | E_{d} |
| 3551 | E₁ₐE₂E₃EᵢᵥE₄ | Eᵥᵢᵢ | E₄* | E_{d} |
| 3552 | E_{1b}E₂E₃EᵢᵥE₄ | Eᵥᵢᵢ | E₄* | E_{d} |
| 3553 | E₁ₐE₂ | Eᵥᵢᵢ | E₅ | E_{d} |
| 3554 | E_{1b}E₂ | Eᵥᵢᵢ | E₅ | E_{d} |
| 3555 | E₁ₐE₂E₃ | Eᵥᵢᵢ | E₅ | E_{d} |
| 3556 | E_{1b}E₂E₃ | Eᵥᵢᵢ | E₅ | E_{d} |
| 3557 | E₁ₐE₂Eᵢ | Eᵥᵢᵢ | E₅ | E_{d} |
| 3558 | E_{1b}E₂Eᵢ | Eᵥᵢᵢ | E₅ | E_{d} |
| 3559 | E₁ₐE₂Eᵢᵢ | Eᵥᵢᵢ | E₅ | E_{d} |
| 3560 | E_{1b}E₂Eᵢᵢ | Eᵥᵢᵢ | E₅ | E_{d} |
| 3561 | E₁ₐE₂EᵢᵢE_{iib} | Eᵥᵢᵢ | E₅ | E_{d} |
| 3562 | E_{1b}E₂EᵢᵢE_{iib} | Eᵥᵢᵢ | E₅ | E_{d} |
| 3563 | E₁ₐE₂E₃Eᵢ | Eᵥᵢᵢ | E₅ | E_{d} |
| 3564 | E_{1b}E₂E₃Eᵢ | Eᵥᵢᵢ | E₅ | E_{d} |
| 3565 | E₁ₐE₂E₃Eᵢᵢ | Eᵥᵢᵢ | E₅ | E_{d} |
| 3566 | E_{1b}E₂E₃Eᵢᵢ | Eᵥᵢᵢ | E₅ | E_{d} |
| 3567 | E₁ₐE₂E₃EᵢᵢE_{iib} | Eᵥᵢᵢ | E₅ | E_{d} |
| 3568 | E_{1b}E₂E₃EᵢᵢE_{iib} | Eᵥᵢᵢ | E₅ | E_{d} |
| 3569 | E₁ₐE₂E₄ | Eᵥᵢᵢ | E₅ | E_{d} |
| 3570 | E_{1b}E₂E₄ | Eᵥᵢᵢ | E₅ | E_{d} |
| 3571 | E₁ₐE₂E₃E₄ | Eᵥᵢᵢ | E₅ | E_{d} |
| 3572 | E_{1b}E₂E₃E₄ | Eᵥᵢᵢ | E₅ | E_{d} |
| 3573 | E₁ₐE₂EᵢE₄ | Eᵥᵢᵢ | E₅ | E_{d} |
| 3574 | E_{1b}E₂EᵢE₄ | Eᵥᵢᵢ | E₅ | E_{d} |
| 3575 | E₁ₐE₂EᵢᵢE₄ | Eᵥᵢᵢ | E₅ | E_{d} |
| 3576 | E_{1b}E₂EᵢᵢE₄ | Eᵥᵢᵢ | E₅ | E_{d} |
| 3577 | E₁ₐE₂EᵢᵢE_{iib}E₄ | Eᵥᵢᵢ | E₅ | E_{d} |
| 3578 | E_{1b}E₂EᵢᵢE_{iib}E₄ | Eᵥᵢᵢ | E₅ | E_{d} |
| 3579 | E₁ₐE₂E₃EᵢE₄ | Eᵥᵢᵢ | E₅ | E_{d} |
| 3580 | E_{1b}E₂E₃EᵢE₄ | Eᵥᵢᵢ | E₅ | E_{d} |
| 3581 | E₁ₐE₂E₃EᵢᵢE₄ | Eᵥᵢᵢ | E₅ | E_{d} |
| 3582 | E_{1b}E₂E₃EᵢᵢE₄ | Eᵥᵢᵢ | E₅ | E_{d} |
| 3583 | E₁ₐE₂E₃EᵢᵢE_{iib}E₄ | Eᵥᵢᵢ | E₅ | E_{d} |
| 3584 | E_{1b}E₂E₃EᵢᵢE_{iib}E₄ | Eᵥᵢᵢ | E₅ | E_{d} |
| 3585 | E₁ₐE₂Eᵢᵢᵢ | Eᵥᵢᵢ | E₅ | E_{d} |
| 3586 | E_{1b}E₂Eᵢᵢᵢ | Eᵥᵢᵢ | E₅ | E_{d} |
| 3587 | E₁ₐE₂E₃Eᵢᵢᵢ | Eᵥᵢᵢ | E₅ | E_{d} |
| 3588 | E_{1b}E₂E₃Eᵢᵢᵢ | Eᵥᵢᵢ | E₅ | E_{d} |
| 3589 | E₁ₐE₂EᵢᵢᵢE₄ | Eᵥᵢᵢ | E₅ | E_{d} |
| 3590 | E_{1b}E₂EᵢᵢᵢE₄ | Eᵥᵢᵢ | E₅ | E_{d} |
| 3591 | E₁ₐE₂E₃EᵢᵢᵢE₄ | Eᵥᵢᵢ | E₅ | E_{d} |
| 3592 | E_{1b}E₂E₃EᵢᵢᵢE₄ | Eᵥᵢᵢ | E₅ | E_{d} |
| 3593 | E₁ₐE₂Eᵢᵥ | Eᵥᵢᵢ | E₅ | E_{d} |
| 3594 | E_{1b}E₂Eᵢᵥ | Eᵥᵢᵢ | E₅ | E_{d} |
| 3595 | E₁ₐE₂E₃Eᵢᵥ | Eᵥᵢᵢ | E₅ | E_{d} |
| 3596 | E_{1b}E₂E₃Eᵢᵥ | Eᵥᵢᵢ | E₅ | E_{d} |
| 3597 | E₁ₐE₂EᵢᵥE₄ | Eᵥᵢᵢ | E₅ | E_{d} |
| 3598 | E_{1b}E₂EᵢᵥE₄ | Eᵥᵢᵢ | E₅ | E_{d} |
| 3599 | E₁ₐE₂E₃EᵢᵥE₄ | Eᵥᵢᵢ | E₅ | E_{d} |
| 3600 | E_{1b}E₂E₃EᵢᵥE₄ | Eᵥᵢᵢ | E₅ | E_{d} |
| 3601 | E₁ₐE₂ | Eᵥᵢᵢ | E₄*E₅ | E_{d} |
| 3602 | E_{1b}E₂ | Eᵥᵢᵢ | E₄*E₅ | E_{d} |
| 3603 | E₁ₐE₂E₃ | Eᵥᵢᵢ | E₄*E₅ | E_{d} |
| 3604 | E_{1b}E₂E₃ | Eᵥᵢᵢ | E₄*E₅ | E_{d} |
| 3605 | E₁ₐE₂Eᵢ | Eᵥᵢᵢ | E₄*E₅ | E_{d} |
| 3606 | E_{1b}E₂Eᵢ | Eᵥᵢᵢ | E₄*E₅ | E_{d} |
| 3607 | E₁ₐE₂Eᵢᵢ | Eᵥᵢᵢ | E₄*E₅ | E_{d} |
| 3608 | E_{1b}E₂Eᵢᵢ | Eᵥᵢᵢ | E₄*E₅ | E_{d} |
| 3609 | E₁ₐE₂EᵢᵢE_{iib} | Eᵥᵢᵢ | E₄*E₅ | E_{d} |
| 3610 | E_{1b}E₂EᵢᵢE_{iib} | Eᵥᵢᵢ | E₄*E₅ | E_{d} |
| 3611 | E₁ₐE₂E₃Eᵢ | Eᵥᵢᵢ | E₄*E₅ | E_{d} |
| 3612 | E_{1b}E₂E₃Eᵢ | Eᵥᵢᵢ | E₄*E₅ | E_{d} |
| 3613 | E₁ₐE₂E₃Eᵢᵢ | Eᵥᵢᵢ | E₄*E₅ | E_{d} |
| 3614 | E_{1b}E₂E₃Eᵢᵢ | Eᵥᵢᵢ | E₄*E₅ | E_{d} |
| 3615 | E₁ₐE₂E₃EᵢᵢE_{iib} | Eᵥᵢᵢ | E₄*E₅ | E_{d} |
| 3616 | E_{1b}E₂E₃EᵢᵢE_{iib} | Eᵥᵢᵢ | E₄*E₅ | E_{d} |
| 3617 | E₁ₐE₂E₄ | Eᵥᵢᵢ | E₄*E₅ | E_{d} |
| 3618 | E_{1b}E₂E₄ | Eᵥᵢᵢ | E₄*E₅ | E_{d} |
| 3619 | E₁ₐE₂E₃E₄ | Eᵥᵢᵢ | E₄*E₅ | E_{d} |
| 3620 | E_{1b}E₂E₃E₄ | Eᵥᵢᵢ | E₄*E₅ | E_{d} |
| 3621 | E₁ₐE₂EᵢE₄ | Eᵥᵢᵢ | E₄*E₅ | E_{d} |
| 3622 | E_{1b}E₂EᵢE₄ | Eᵥᵢᵢ | E₄*E₅ | E_{d} |
| 3623 | E₁ₐE₂EᵢᵢE₄ | Eᵥᵢᵢ | E₄*E₅ | E_{d} |
| 3624 | E_{1b}E₂EᵢᵢE₄ | Eᵥᵢᵢ | E₄*E₅ | E_{d} |
| 3625 | E₁ₐE₂EᵢᵢE_{iib}E₄ | Eᵥᵢᵢ | E₄*E₅ | E_{d} |
| 3626 | E_{1b}E₂EᵢᵢE_{iib}E₄ | Eᵥᵢᵢ | E₄*E₅ | E_{d} |
| 3627 | E₁ₐE₂E₃EᵢE₄ | Eᵥᵢᵢ | E₄*E₅ | E_{d} |
| 3628 | E_{1b}E₂E₃EᵢE₄ | Eᵥᵢᵢ | E₄*E₅ | E_{d} |
| 3629 | E₁ₐE₂E₃EᵢᵢE₄ | Eᵥᵢᵢ | E₄*E₅ | E_{d} |
| 3630 | E_{1b}E₂E₃EᵢᵢE₄ | Eᵥᵢᵢ | E₄*E₅ | E_{d} |
| 3631 | E₁ₐE₂E₃EᵢᵢE_{iib}E₄ | Eᵥᵢᵢ | E₄*E₅ | E_{d} |
| 3632 | E_{1b}E₂E₃EᵢᵢE_{iib}E₄ | Eᵥᵢᵢ | E₄*E₅ | E_{d} |
| 3633 | E₁ₐE₂Eᵢᵢᵢ | Eᵥᵢᵢ | E₄*E₅ | E_{d} |
| 3634 | E_{1b}E₂Eᵢᵢᵢ | Eᵥᵢᵢ | E₄*E₅ | E_{d} |
| 3635 | E₁ₐE₂E₃Eᵢᵢᵢ | Eᵥᵢᵢ | E₄*E₅ | E_{d} |
| 3636 | E_{1b}E₂E₃Eᵢᵢᵢ | Eᵥᵢᵢ | E₄*E₅ | E_{d} |
| 3637 | E₁ₐE₂EᵢᵢᵢE₄ | Eᵥᵢᵢ | E₄*E₅ | E_{d} |
| 3638 | E_{1b}E₂EᵢᵢᵢE₄ | Eᵥᵢᵢ | E₄*E₅ | E_{d} |
| 3639 | E₁ₐE₂E₃EᵢᵢᵢE₄ | Eᵥᵢᵢ | E₄*E₅ | E_{d} |
| 3640 | E_{1b}E₂E₃EᵢᵢᵢE₄ | Eᵥᵢᵢ | E₄*E₅ | E_{d} |
| 3641 | E₁ₐE₂Eᵢᵥ | Eᵥᵢᵢ | E₄*E₅ | E_{d} |
| 3642 | E_{1b}E₂Eᵢᵥ | Eᵥᵢᵢ | E₄*E₅ | E_{d} |
| 3643 | E₁ₐE₂E₃Eᵢᵥ | Eᵥᵢᵢ | E₄*E₅ | E_{d} |
| 3644 | E_{1b}E₂E₃Eᵢᵥ | Eᵥᵢᵢ | E₄*E₅ | E_{d} |
| 3645 | E₁ₐE₂EᵢᵥE₄ | Eᵥᵢᵢ | E₄*E₅ | E_{d} |
| 3646 | E_{1b}E₂EᵢᵥE₄ | Eᵥᵢᵢ | E₄*E₅ | E_{d} |
| 3647 | E₁ₐE₂E₃EᵢᵥE₄ | Eᵥᵢᵢ | E₄*E₅ | E_{d} |
| 3648 | E_{1b}E₂E₃EᵢᵥE₄ | Eᵥᵢᵢ | E₄*E₅ | E_{d} |
| 3649 | E₁ₐE₂ | AlkLEᵥᵢᵢ | | E_{d} |
| 3650 | E_{1b}E₂ | AlkLEᵥᵢᵢ | | E_{d} |
| 3651 | E₁ₐE₂E₃ | AlkLEᵥᵢᵢ | | E_{d} |
| 3652 | E_{1b}E₂E₃ | AlkLEᵥᵢᵢ | | E_{d} |
| 3653 | E₁ₐE₂Eᵢ | AlkLEᵥᵢᵢ | | E_{d} |
| 3654 | E_{1b}E₂Eᵢ | AlkLEᵥᵢᵢ | | E_{d} |
| 3655 | E₁ₐE₂Eᵢᵢ | AlkLEᵥᵢᵢ | | E_{d} |
| 3656 | E_{1b}E₂Eᵢᵢ | AlkLEᵥᵢᵢ | | E_{d} |
| 3657 | E₁ₐE₂EᵢᵢE_{iib} | AlkLEᵥᵢᵢ | | E_{d} |
| 3658 | E_{1b}E₂EᵢᵢE_{iib} | AlkLEᵥᵢᵢ | | E_{d} |
| 3659 | E₁ₐE₂E₃Eᵢ | AlkLEᵥᵢᵢ | | E_{d} |
| 3660 | E_{1b}E₂E₃Eᵢ | AlkLEᵥᵢᵢ | | E_{d} |
| 3661 | E₁ₐE₂E₃Eᵢᵢ | AlkLEᵥᵢᵢ | | E_{d} |
| 3662 | E_{1b}E₂E₃Eᵢᵢ | AlkLEᵥᵢᵢ | | E_{d} |
| 3663 | E₁ₐE₂E₃EᵢᵢE_{iib} | AlkLEᵥᵢᵢ | | E_{d} |
| 3664 | E_{1b}E₂E₃EᵢᵢE_{iib} | AlkLEᵥᵢᵢ | | E_{d} |
| 3665 | E₁ₐE₂E₄ | AlkLEᵥᵢᵢ | | E_{d} |
| 3666 | E_{1b}E₂E₄ | AlkLEᵥᵢᵢ | | E_{d} |
| 3667 | E₁ₐE₂E₃E₄ | AlkLEᵥᵢᵢ | | E_{d} |
| 3668 | E_{1b}E₂E₃E₄ | AlkLEᵥᵢᵢ | | E_{d} |
| 3669 | E₁ₐE₂EᵢE₄ | AlkLEᵥᵢᵢ | | E_{d} |
| 3670 | E_{1b}E₂EᵢE₄ | AlkLEᵥᵢᵢ | | E_{d} |
| 3671 | E₁ₐE₂EᵢᵢE₄ | AlkLEᵥᵢᵢ | | E_{d} |
| 3672 | E_{1b}E₂EᵢᵢE₄ | AlkLEᵥᵢᵢ | | E_{d} |
| 3673 | E₁ₐE₂EᵢᵢE_{iib}E₄ | AlkLEᵥᵢᵢ | | E_{d} |
| 3674 | E_{1b}E₂EᵢᵢE_{iib}E₄ | AlkLEᵥᵢᵢ | | E_{d} |
| 3675 | E₁ₐE₂E₃EᵢE₄ | AlkLEᵥᵢᵢ | | E_{d} |
| 3676 | E_{1b}E₂E₃EᵢE₄ | AlkLEᵥᵢᵢ | | E_{d} |
| 3677 | E₁ₐE₂E₃EᵢᵢE₄ | AlkLEᵥᵢᵢ | | E_{d} |
| 3678 | E_{1b}E₂E₃EᵢᵢE₄ | AlkLEᵥᵢᵢ | | E_{d} |
| 3679 | E₁ₐE₂E₃EᵢᵢE_{iib}E₄ | AlkLEᵥᵢᵢ | | E_{d} |
| 3680 | E_{1b}E₂E₃EᵢᵢE_{iib}E₄ | AlkLEᵥᵢᵢ | | E_{d} |
| 3681 | E₁ₐE₂Eᵢᵢᵢ | AlkLEᵥᵢᵢ | | E_{d} |
| 3682 | E_{1b}E₂Eᵢᵢᵢ | AlkLEᵥᵢᵢ | | E_{d} |
| 3683 | E₁ₐE₂E₃Eᵢᵢᵢ | AlkLEᵥᵢᵢ | | E_{d} |
| 3684 | E_{1b}E₂E₃Eᵢᵢᵢ | AlkLEᵥᵢᵢ | | E_{d} |
| 3685 | E₁ₐE₂EᵢᵢᵢE₄ | AlkLEᵥᵢᵢ | | E_{d} |
| 3686 | E_{1b}E₂EᵢᵢᵢE₄ | AlkLEᵥᵢᵢ | | E_{d} |
| 3687 | E₁ₐE₂E₃EᵢᵢᵢE₄ | AlkLEᵥᵢᵢ | | E_{d} |
| 3688 | E_{1b}E₂E₃EᵢᵢᵢE₄ | AlkLEᵥᵢᵢ | | E_{d} |
| 3689 | E₁ₐE₂Eᵢᵥ | AlkLEᵥᵢᵢ | | E_{d} |
| 3690 | E_{1b}E₂Eᵢᵥ | AlkLEᵥᵢᵢ | | E_{d} |
| 3691 | E₁ₐE₂E₃Eᵢᵥ | AlkLEᵥᵢᵢ | | E_{d} |
| 3692 | E_{1b}E₂E₃Eᵢᵥ | AlkLEᵥᵢᵢ | | E_{d} |
| 3693 | E₁ₐE₂EᵢᵥE₄ | AlkLEᵥᵢᵢ | | E_{d} |
| 3694 | E_{1b}E₂EᵢᵥE₄ | AlkLEᵥᵢᵢ | | E_{d} |
| 3695 | E₁ₐE₂E₃EᵢᵥE₄ | AlkLEᵥᵢᵢ | | E_{d} |
| 3696 | E_{1b}E₂E₃EᵢᵥE₄ | AlkLEᵥᵢᵢ | | E_{d} |
| 3697 | E₁ₐE₂ | AlkLEᵥᵢᵢ | E₄* | E_{d} |
| 3698 | E_{1b}E₂ | AlkLEᵥᵢᵢ | E₄* | E_{d} |
| 3699 | E₁ₐE₂E₃ | AlkLEᵥᵢᵢ | E₄* | E_{d} |
| 3700 | E_{1b}E₂E₃ | AlkLEᵥᵢᵢ | E₄* | E_{d} |
| 3701 | E₁ₐE₂Eᵢ | AlkLEᵥᵢᵢ | E₄* | E_{d} |
| 3702 | E_{1b}E₂Eᵢ | AlkLEᵥᵢᵢ | E₄* | E_{d} |
| 3703 | E₁ₐE₂Eᵢᵢ | AlkLEᵥᵢᵢ | E₄* | E_{d} |
| 3704 | E_{1b}E₂Eᵢᵢ | AlkLEᵥᵢᵢ | E₄* | E_{d} |
| 3705 | E₁ₐE₂EᵢᵢE_{iib} | AlkLEᵥᵢᵢ | E₄* | E_{d} |
| 3706 | E_{1b}E₂EᵢᵢE_{iib} | AlkLEᵥᵢᵢ | E₄* | E_{d} |
| 3707 | E₁ₐE₂E₃Eᵢ | AlkLEᵥᵢᵢ | E₄* | E_{d} |
| 3708 | E_{1b}E₂E₃Eᵢ | AlkLEᵥᵢᵢ | E₄* | E_{d} |
| 3709 | E₁ₐE₂E₃Eᵢᵢ | AlkLEᵥᵢᵢ | E₄* | E_{d} |
| 3710 | E_{1b}E₂E₃Eᵢᵢ | AlkLEᵥᵢᵢ | E₄* | E_{d} |
| 3711 | E₁ₐE₂E₃EᵢᵢE_{iib} | AlkLEᵥᵢᵢ | E₄* | E_{d} |
| 3712 | E_{1b}E₂E₃EᵢᵢE_{iib} | AlkLEᵥᵢᵢ | E₄* | E_{d} |
| 3713 | E₁ₐE₂E₄ | AlkLEᵥᵢᵢ | E₄* | E_{d} |
| 3714 | E_{1b}E₂E₄ | AlkLEᵥᵢᵢ | E₄* | E_{d} |
| 3715 | E₁ₐE₂E₃E₄ | AlkLEᵥᵢᵢ | E₄* | E_{d} |
| 3716 | E_{1b}E₂E₃E₄ | AlkLEᵥᵢᵢ | E₄* | E_{d} |
| 3717 | E₁ₐE₂EᵢE₄ | AlkLEᵥᵢᵢ | E₄* | E_{d} |
| 3718 | E_{1b}E₂EᵢE₄ | AlkLEᵥᵢᵢ | E₄* | E_{d} |
| 3719 | E₁ₐE₂EᵢᵢE₄ | AlkLEᵥᵢᵢ | E₄* | E_{d} |
| 3720 | E_{1b}E₂EᵢᵢE₄ | AlkLEᵥᵢᵢ | E₄* | E_{d} |
| 3721 | E₁ₐE₂EᵢᵢE_{iib}E₄ | AlkLEᵥᵢᵢ | E₄* | E_{d} |
| 3722 | E_{1b}E₂EᵢᵢE_{iib}E₄ | AlkLEᵥᵢᵢ | E₄* | E_{d} |
| 3723 | E₁ₐE₂E₃EᵢE₄ | AlkLEᵥᵢᵢ | E₄* | E_{d} |
| 3724 | E_{1b}E₂E₃EᵢE₄ | AlkLEᵥᵢᵢ | E₄* | E_{d} |
| 3725 | E₁ₐE₂E₃EᵢᵢE₄ | AlkLEᵥᵢᵢ | E₄* | E_{d} |
| 3726 | E_{1b}E₂E₃EᵢᵢE₄ | AlkLEᵥᵢᵢ | E₄* | E_{d} |
| 3727 | E₁ₐE₂E₃EᵢᵢE_{iib}E₄ | AlkLEᵥᵢᵢ | E₄* | E_{d} |
| 3728 | E_{1b}E₂E₃EᵢᵢE_{iib}E₄ | AlkLEᵥᵢᵢ | E₄* | E_{d} |
| 3729 | E₁ₐE₂Eᵢᵢᵢ | AlkLEᵥᵢᵢ | E₄* | E_{d} |
| 3730 | E_{1b}E₂Eᵢᵢᵢ | AlkLEᵥᵢᵢ | E₄* | E_{d} |
| 3731 | E₁ₐE₂E₃Eᵢᵢᵢ | AlkLEᵥᵢᵢ | E₄* | E_{d} |
| 3732 | E_{1b}E₂E₃Eᵢᵢᵢ | AlkLEᵥᵢᵢ | E₄* | E_{d} |
| 3733 | E₁ₐE₂EᵢᵢᵢE₄ | AlkLEᵥᵢᵢ | E₄* | E_{d} |
| 3734 | E_{1b}E₂EᵢᵢᵢE₄ | AlkLEᵥᵢᵢ | E₄* | E_{d} |
| 3735 | E₁ₐE₂E₃EᵢᵢᵢE₄ | AlkLEᵥᵢᵢ | E₄* | E_{d} |
| 3736 | E_{1b}E₂E₃EᵢᵢᵢE₄ | AlkLEᵥᵢᵢ | E₄* | E_{d} |
| 3737 | E₁ₐE₂Eᵢᵥ | AlkLEᵥᵢᵢ | E₄* | E_{d} |
| 3738 | E_{1b}E₂Eᵢᵥ | AlkLEᵥᵢᵢ | E₄* | E_{d} |
| 3739 | E₁ₐE₂E₃Eᵢᵥ | AlkLEᵥᵢᵢ | E₄* | E_{d} |
| 3740 | E_{1b}E₂E₃Eᵢᵥ | AlkLEᵥᵢᵢ | E₄* | E_{d} |
| 3741 | E₁ₐE₂EᵢᵥE₄ | AlkLEᵥᵢᵢ | E₄* | E_{d} |
| 3742 | E_{1b}E₂EᵢᵥE₄ | AlkLEᵥᵢᵢ | E₄* | E_{d} |
| 3743 | E₁ₐE₂E₃EᵢᵥE₄ | AlkLEᵥᵢᵢ | E₄* | E_{d} |
| 3744 | E_{1b}E₂E₃EᵢᵥE₄ | AlkLEᵥᵢᵢ | E₄* | E_{d} |
| 3745 | E₁ₐE₂ | AlkLEᵥᵢᵢ | E₅ | E_{d} |
| 3746 | E_{1b}E₂ | AlkLEᵥᵢᵢ | E₅ | E_{d} |
| 3747 | E₁ₐE₂E₃ | AlkLEᵥᵢᵢ | E₅ | E_{d} |
| 3748 | E_{1b}E₂E₃ | AlkLEᵥᵢᵢ | E₅ | E_{d} |
| 3749 | E₁ₐE₂Eᵢ | AlkLEᵥᵢᵢ | E₅ | E_{d} |
| 3750 | E_{1b}E₂Eᵢ | AlkLEᵥᵢᵢ | E₅ | E_{d} |
| 3751 | E₁ₐE₂Eᵢᵢ | AlkLEᵥᵢᵢ | E₅ | E_{d} |
| 3752 | E_{1b}E₂Eᵢᵢ | AlkLEᵥᵢᵢ | E₅ | E_{d} |
| 3753 | E₁ₐE₂EᵢᵢE_{iib} | AlkLEᵥᵢᵢ | E₅ | E_{d} |
| 3754 | E_{1b}E₂EᵢᵢE_{iib} | AlkLEᵥᵢᵢ | E₅ | E_{d} |
| 3755 | E₁ₐE₂E₃Eᵢ | AlkLEᵥᵢᵢ | E₅ | E_{d} |
| 3756 | E_{1b}E₂E₃Eᵢ | AlkLEᵥᵢᵢ | E₅ | E_{d} |
| 3757 | E₁ₐE₂E₃Eᵢᵢ | AlkLEᵥᵢᵢ | E₅ | E_{d} |
| 3758 | E_{1b}E₂E₃Eᵢᵢ | AlkLEᵥᵢᵢ | E₅ | E_{d} |
| 3759 | E₁ₐE₂E₃EᵢᵢE_{iib} | AlkLEᵥᵢᵢ | E₅ | E_{d} |
| 3760 | E_{1b}E₂E₃EᵢᵢE_{iib} | AlkLEᵥᵢᵢ | E₅ | E_{d} |
| 3761 | E₁ₐE₂E₄ | AlkLEᵥᵢᵢ | E₅ | E_{d} |
| 3762 | E_{1b}E₂E₄ | AlkLEᵥᵢᵢ | E₅ | E_{d} |
| 3763 | E₁ₐE₂E₃E₄ | AlkLEᵥᵢᵢ | E₅ | E_{d} |
| 3764 | E_{1b}E₂E₃E₄ | AlkLEᵥᵢᵢ | E₅ | E_{d} |
| 3765 | E₁ₐE₂EᵢE₄ | AlkLEᵥᵢᵢ | E₅ | E_{d} |
| 3766 | E_{1b}E₂EᵢE₄ | AlkLEᵥᵢᵢ | E₅ | E_{d} |
| 3767 | E₁ₐE₂EᵢᵢE₄ | AlkLEᵥᵢᵢ | E₅ | E_{d} |
| 3768 | E_{1b}E₂EᵢᵢE₄ | AlkLEᵥᵢᵢ | E₅ | E_{d} |
| 3769 | E₁ₐE₂EᵢᵢE_{iib}E₄ | AlkLEᵥᵢᵢ | E₅ | E_{d} |
| 3770 | E_{1b}E₂EᵢᵢE_{iib}E₄ | AlkLEᵥᵢᵢ | E₅ | E_{d} |
| 3771 | E₁ₐE₂E₃EᵢE₄ | AlkLEᵥᵢᵢ | E₅ | E_{d} |
| 3772 | E_{1b}E₂E₃EᵢE₄ | AlkLEᵥᵢᵢ | E₅ | E_{d} |
| 3773 | E₁ₐE₂E₃EᵢᵢE₄ | AlkLEᵥᵢᵢ | E₅ | E_{d} |
| 3774 | E_{1b}E₂E₃EᵢᵢE₄ | AlkLEᵥᵢᵢ | E₅ | E_{d} |
| 3775 | E₁ₐE₂E₃EᵢᵢE_{iib}E₄ | AlkLEᵥᵢᵢ | E₅ | E_{d} |
| 3776 | E_{1b}E₂E₃EᵢᵢE_{iib}E₄ | AlkLEᵥᵢᵢ | E₅ | E_{d} |
| 3777 | E₁ₐE₂Eᵢᵢᵢ | AlkLEᵥᵢᵢ | E₅ | E_{d} |
| 3778 | E_{1b}E₂Eᵢᵢᵢ | AlkLEᵥᵢᵢ | E₅ | E_{d} |
| 3779 | E₁ₐE₂E₃Eᵢᵢᵢ | AlkLEᵥᵢᵢ | E₅ | E_{d} |
| 3780 | E_{1b}E₂E₃Eᵢᵢᵢ | AlkLEᵥᵢᵢ | E₅ | E_{d} |
| 3781 | E₁ₐE₂EᵢᵢᵢE₄ | AlkLEᵥᵢᵢ | E₅ | E_{d} |
| 3782 | E_{1b}E₂EᵢᵢᵢE₄ | AlkLEᵥᵢᵢ | E₅ | E_{d} |
| 3783 | E₁ₐE₂E₃EᵢᵢᵢE₄ | AlkLEᵥᵢᵢ | E₅ | E_{d} |
| 3784 | E_{1b}E₂E₃EᵢᵢᵢE₄ | AlkLEᵥᵢᵢ | E₅ | E_{d} |
| 3785 | E₁ₐE₂Eᵢᵥ | AlkLEᵥᵢᵢ | E₅ | E_{d} |
| 3786 | E_{1b}E₂Eᵢᵥ | AlkLEᵥᵢᵢ | E₅ | E_{d} |
| 3787 | E₁ₐE₂E₃Eᵢᵥ | AlkLEᵥᵢᵢ | E₅ | E_{d} |
| 3788 | E_{1b}E₂E₃Eᵢᵥ | AlkLEᵥᵢᵢ | E₅ | E_{d} |
| 3789 | E₁ₐE₂EᵢᵥE₄ | AlkLEᵥᵢᵢ | E₅ | E_{d} |
| 3790 | E_{1b}E₂EᵢᵥE₄ | AlkLEᵥᵢᵢ | E₅ | E_{d} |
| 3791 | E₁ₐE₂E₃EᵢᵥE₄ | AlkLEᵥᵢᵢ | E₅ | E_{d} |
| 3792 | E_{1b}E₂E₃EᵢᵥE₄ | AlkLEᵥᵢᵢ | E₅ | E_{d} |
| 3793 | E₁ₐE₂ | AlkLEᵥᵢᵢ | E₄*E₅ | E_{d} |
| 3794 | E_{1b}E₂ | AlkLEᵥᵢᵢ | E₄*E₅ | E_{d} |
| 3795 | E₁ₐE₂E₃ | AlkLEᵥᵢᵢ | E₄*E₅ | E_{d} |
| 3796 | E_{1b}E₂E₃ | AlkLEᵥᵢᵢ | E₄*E₅ | E_{d} |
| 3797 | E₁ₐE₂Eᵢ | AlkLEᵥᵢᵢ | E₄*E₅ | E_{d} |
| 3798 | E_{1b}E₂Eᵢ | AlkLEᵥᵢᵢ | E₄*E₅ | E_{d} |
| 3799 | E₁ₐE₂Eᵢᵢ | AlkLEᵥᵢᵢ | E₄*E₅ | E_{d} |
| 3800 | E_{1b}E₂Eᵢᵢ | AlkLEᵥᵢᵢ | E₄*E₅ | E_{d} |
| 3801 | E₁ₐE₂EᵢᵢE_{iib} | AlkLEᵥᵢᵢ | E₄*E₅ | E_{d} |
| 3802 | E_{1b}E₂EᵢᵢE_{iib} | AlkLEᵥᵢᵢ | E₄*E₅ | E_{d} |
| 3803 | E₁ₐE₂E₃Eᵢ | AlkLEᵥᵢᵢ | E₄*E₅ | E_{d} |
| 3804 | E_{1b}E₂E₃Eᵢ | AlkLEᵥᵢᵢ | E₄*E₅ | E_{d} |
| 3805 | E₁ₐE₂E₃Eᵢᵢ | AlkLEᵥᵢᵢ | E₄*E₅ | E_{d} |
| 3806 | E_{1b}E₂E₃Eᵢᵢ | AlkLEᵥᵢᵢ | E₄*E₅ | E_{d} |
| 3807 | E₁ₐE₂E₃EᵢᵢE_{iib} | AlkLEᵥᵢᵢ | E₄*E₅ | E_{d} |
| 3808 | E_{1b}E₂E₃EᵢᵢE_{iib} | AlkLEᵥᵢᵢ | E₄*E₅ | E_{d} |
| 3809 | E₁ₐE₂E₄ | AlkLEᵥᵢᵢ | E₄*E₅ | E_{d} |
| 3810 | E_{1b}E₂E₄ | AlkLEᵥᵢᵢ | E₄*E₅ | E_{d} |
| 3811 | E₁ₐE₂E₃E₄ | AlkLEᵥᵢᵢ | E₄*E₅ | E_{d} |
| 3812 | E_{1b}E₂E₃E₄ | AlkLEᵥᵢᵢ | E₄*E₅ | E_{d} |
| 3813 | E₁ₐE₂EᵢE₄ | AlkLEᵥᵢᵢ | E₄*E₅ | E_{d} |
| 3814 | E_{1b}E₂EᵢE₄ | AlkLEᵥᵢᵢ | E₄*E₅ | E_{d} |
| 3815 | E₁ₐE₂EᵢᵢE₄ | AlkLEᵥᵢᵢ | E₄*E₅ | E_{d} |
| 3816 | E_{1b}E₂EᵢᵢE₄ | AlkLEᵥᵢᵢ | E₄*E₅ | E_{d} |
| 3817 | E₁ₐE₂EᵢᵢE_{iib}E₄ | AlkLEᵥᵢᵢ | E₄*E₅ | E_{d} |
| 3818 | E_{1b}E₂EᵢᵢE_{iib}E₄ | AlkLEᵥᵢᵢ | E₄*E₅ | E_{d} |
| 3819 | E₁ₐE₂E₃EᵢE₄ | AlkLEᵥᵢᵢ | E₄*E₅ | E_{d} |
| 3820 | E_{1b}E₂E₃EᵢE₄ | AlkLEᵥᵢᵢ | E₄*E₅ | E_{d} |
| 3821 | E₁ₐE₂E₃EᵢᵢE₄ | AlkLEᵥᵢᵢ | E₄*E₅ | E_{d} |
| 3822 | E_{1b}E₂E₃EᵢᵢE₄ | AlkLEᵥᵢᵢ | E₄*E₅ | E_{d} |
| 3823 | E₁ₐE₂E₃EᵢᵢE_{iib}E₄ | AlkLEᵥᵢᵢ | E₄*E₅ | E_{d} |
| 3824 | E_{1b}E₂E₃EᵢᵢE_{iib}E₄ | AlkLEᵥᵢᵢ | E₄*E₅ | E_{d} |
| 3825 | E₁ₐE₂Eᵢᵢᵢ | AlkLEᵥᵢᵢ | E₄*E₅ | E_{d} |
| 3826 | E_{1b}E₂Eᵢᵢᵢ | AlkLEᵥᵢᵢ | E₄*E₅ | E_{d} |
| 3827 | E₁ₐE₂E₃Eᵢᵢᵢ | AlkLEᵥᵢᵢ | E₄*E₅ | E_{d} |
| 3828 | E_{1b}E₂E₃Eᵢᵢᵢ | AlkLEᵥᵢᵢ | E₄*E₅ | E_{d} |
| 3829 | E₁ₐE₂EᵢᵢᵢE₄ | AlkLEᵥᵢᵢ | E₄*E₅ | E_{d} |
| 3830 | E_{1b}E₂EᵢᵢᵢE₄ | AlkLEᵥᵢᵢ | E₄*E₅ | E_{d} |
| 3831 | E₁ₐE₂E₃EᵢᵢᵢE₄ | AlkLEᵥᵢᵢ | E₄*E₅ | E_{d} |
| 3832 | E_{1b}E₂E₃EᵢᵢᵢE₄ | AlkLEᵥᵢᵢ | E₄*E₅ | E_{d} |
| 3833 | E₁ₐE₂Eᵢᵥ | AlkLEᵥᵢᵢ | E₄*E₅ | E_{d} |
| 3834 | E_{1b}E₂Eᵢᵥ | AlkLEᵥᵢᵢ | E₄*E₅ | E_{d} |
| 3835 | E₁ₐE₂E₃Eᵢᵥ | AlkLEᵥᵢᵢ | E₄*E₅ | E_{d} |
| 3836 | E_{1b}E₂E₃Eᵢᵥ | AlkLEᵥᵢᵢ | E₄*E₅ | E_{d} |
| 3837 | E₁ₐE₂EᵢᵥE₄ | AlkLEᵥᵢᵢ | E₄*E₅ | E_{d} |
| 3838 | E_{1b}E₂EᵢᵥE₄ | AlkLEᵥᵢᵢ | E₄*E₅ | E_{d} |
| 3839 | E₁ₐE₂E₃EᵢᵥE₄ | AlkLEᵥᵢᵢ | E₄*E₅ | E_{d} |
| 3840 | E_{1b}E₂E₃EᵢᵥE₄ | AlkLEᵥᵢᵢ | E₄*E₅ | E_{d} |
| 3841 | E₁ₐE₂ | Eᵥᵢᵢ | | Eₑ |
| 3842 | E_{1b}E₂ | Eᵥᵢᵢ | | Eₑ |
| 3843 | E₁ₐE₂E₃ | Eᵥᵢᵢ | | Eₑ |
| 3844 | E_{1b}E₂E₃ | Eᵥᵢᵢ | | Eₑ |
| 3845 | E₁ₐE₂Eᵢ | Eᵥᵢᵢ | | Eₑ |
| 3846 | E_{1b}E₂Eᵢ | Eᵥᵢᵢ | | Eₑ |
| 3847 | E₁ₐE₂Eᵢᵢ | Eᵥᵢᵢ | | Eₑ |
| 3848 | E_{1b}E₂Eᵢᵢ | Eᵥᵢᵢ | | Eₑ |
| 3849 | E₁ₐE₂EᵢᵢE_{iib} | Eᵥᵢᵢ | | Eₑ |
| 3850 | E_{1b}E₂EᵢᵢE_{iib} | Eᵥᵢᵢ | | Eₑ |
| 3851 | E₁ₐE₂E₃Eᵢ | Eᵥᵢᵢ | | Eₑ |
| 3852 | E_{1b}E₂E₃Eᵢ | Eᵥᵢᵢ | | Eₑ |
| 3853 | E₁ₐE₂E₃Eᵢᵢ | Eᵥᵢᵢ | | Eₑ |
| 3854 | E_{1b}E₂E₃Eᵢᵢ | Eᵥᵢᵢ | | Eₑ |
| 3855 | E₁ₐE₂E₃EᵢᵢE_{iib} | Eᵥᵢᵢ | | Eₑ |
| 3856 | E_{1b}E₂E₃EᵢᵢE_{iib} | Eᵥᵢᵢ | | Eₑ |
| 3857 | E₁ₐE₂E₄ | Eᵥᵢᵢ | | Eₑ |
| 3858 | E_{1b}E₂E₄ | Eᵥᵢᵢ | | Eₑ |
| 3859 | E₁ₐE₂E₃E₄ | Eᵥᵢᵢ | | Eₑ |
| 3860 | E_{1b}E₂E₃E₄ | Eᵥᵢᵢ | | Eₑ |
| 3861 | E₁ₐE₂EᵢE₄ | Eᵥᵢᵢ | | Eₑ |
| 3862 | E_{1b}E₂EᵢE₄ | Eᵥᵢᵢ | | Eₑ |
| 3863 | E₁ₐE₂EᵢᵢE₄ | Eᵥᵢᵢ | | Eₑ |
| 3864 | E_{1b}E₂EᵢᵢE₄ | Eᵥᵢᵢ | | Eₑ |
| 3865 | E₁ₐE₂EᵢᵢE_{iib}E₄ | Eᵥᵢᵢ | | Eₑ |
| 3866 | E_{1b}E₂EᵢᵢE_{iib}E₄ | Eᵥᵢᵢ | | Eₑ |
| 3867 | E₁ₐE₂E₃EᵢE₄ | Eᵥᵢᵢ | | Eₑ |
| 3868 | E_{1b}E₂E₃EᵢE₄ | Eᵥᵢᵢ | | Eₑ |
| 3869 | E₁ₐE₂E₃EᵢᵢE₄ | Eᵥᵢᵢ | | Eₑ |
| 3870 | E_{1b}E₂E₃EᵢᵢE₄ | Eᵥᵢᵢ | | Eₑ |
| 3871 | E₁ₐE₂E₃EᵢᵢE_{iib}E₄ | Eᵥᵢᵢ | | Eₑ |
| 3872 | E_{1b}E₂E₃EᵢᵢE_{iib}E₄ | Eᵥᵢᵢ | | Eₑ |
| 3873 | E₁ₐE₂Eᵢᵢᵢ | Eᵥᵢᵢ | | Eₑ |
| 3874 | E_{1b}E₂Eᵢᵢᵢ | Eᵥᵢᵢ | | Eₑ |
| 3875 | E₁ₐE₂E₃Eᵢᵢᵢ | Eᵥᵢᵢ | | Eₑ |
| 3876 | E_{1b}E₂E₃Eᵢᵢᵢ | Eᵥᵢᵢ | | Eₑ |
| 3877 | E₁ₐE₂EᵢᵢᵢE₄ | Eᵥᵢᵢ | | Eₑ |
| 3878 | E_{1b}E₂EᵢᵢᵢE₄ | Eᵥᵢᵢ | | Eₑ |
| 3879 | E₁ₐE₂E₃EᵢᵢᵢE₄ | Eᵥᵢᵢ | | Eₑ |
| 3880 | E_{1b}E₂E₃EᵢᵢᵢE₄ | Eᵥᵢᵢ | | Eₑ |
| 3881 | E₁ₐE₂Eᵢᵥ | Eᵥᵢᵢ | | Eₑ |
| 3882 | E_{1b}E₂Eᵢᵥ | Eᵥᵢᵢ | | Eₑ |
| 3883 | E₁ₐE₂E₃Eᵢᵥ | Eᵥᵢᵢ | | Eₑ |
| 3884 | E_{1b}E₂E₃Eᵢᵥ | Eᵥᵢᵢ | | Eₑ |
| 3885 | E₁ₐE₂EᵢᵥE₄ | Eᵥᵢᵢ | | Eₑ |
| 3886 | E_{1b}E₂EᵢᵥE₄ | Eᵥᵢᵢ | | Eₑ |
| 3887 | E₁ₐE₂E₃EᵢᵥE₄ | Eᵥᵢᵢ | | Eₑ |
| 3888 | E_{1b}E₂E₃EᵢᵥE₄ | Eᵥᵢᵢ | | Eₑ |
| 3889 | E₁ₐE₂ | Eᵥᵢᵢ | E₄* | Eₑ |
| 3890 | E_{1b}E₂ | Eᵥᵢᵢ | E₄* | Eₑ |
| 3891 | E₁ₐE₂E₃ | Eᵥᵢᵢ | E₄* | Eₑ |
| 3892 | E_{1b}E₂E₃ | Eᵥᵢᵢ | E₄* | Eₑ |
| 3893 | E₁ₐE₂Eᵢ | Eᵥᵢᵢ | E₄* | Eₑ |
| 3894 | E_{1b}E₂Eᵢ | Eᵥᵢᵢ | E₄* | Eₑ |
| 3895 | E₁ₐE₂Eᵢᵢ | Eᵥᵢᵢ | E₄* | Eₑ |
| 3896 | E_{1b}E₂Eᵢᵢ | Eᵥᵢᵢ | E₄* | Eₑ |
| 3897 | E₁ₐE₂EᵢᵢE_{iib} | Eᵥᵢᵢ | E₄* | Eₑ |
| 3898 | E_{1b}E₂EᵢᵢE_{iib} | Eᵥᵢᵢ | E₄* | Eₑ |
| 3899 | E₁ₐE₂E₃Eᵢ | Eᵥᵢᵢ | E₄* | Eₑ |
| 3900 | E_{1b}E₂E₃Eᵢ | Eᵥᵢᵢ | E₄* | Eₑ |
| 3901 | E₁ₐE₂E₃Eᵢᵢ | Eᵥᵢᵢ | E₄* | Eₑ |
| 3902 | E_{1b}E₂E₃Eᵢᵢ | Eᵥᵢᵢ | E₄* | Eₑ |
| 3903 | E₁ₐE₂E₃EᵢᵢE_{iib} | Eᵥᵢᵢ | E₄* | Eₑ |
| 3904 | E_{1b}E₂E₃EᵢᵢE_{iib} | Eᵥᵢᵢ | E₄* | Eₑ |
| 3905 | E₁ₐE₂E₄ | Eᵥᵢᵢ | E₄* | Eₑ |
| 3906 | E_{1b}E₂E₄ | Eᵥᵢᵢ | E₄* | Eₑ |
| 3907 | E₁ₐE₂E₃E₄ | Eᵥᵢᵢ | E₄* | Eₑ |
| 3908 | E_{1b}E₂E₃E₄ | Eᵥᵢᵢ | E₄* | Eₑ |
| 3909 | E₁ₐE₂EᵢE₄ | Eᵥᵢᵢ | E₄* | Eₑ |
| 3910 | E_{1b}E₂EᵢE₄ | Eᵥᵢᵢ | E₄* | Eₑ |
| 3911 | E₁ₐE₂EᵢᵢE₄ | Eᵥᵢᵢ | E₄* | Eₑ |
| 3912 | E_{1b}E₂EᵢᵢE₄ | Eᵥᵢᵢ | E₄* | Eₑ |
| 3913 | E₁ₐE₂EᵢᵢE_{iib}E₄ | Eᵥᵢᵢ | E₄* | Eₑ |
| 3914 | E_{1b}E₂EᵢᵢE_{iib}E₄ | Eᵥᵢᵢ | E₄* | Eₑ |
| 3915 | E₁ₐE₂E₃EᵢE₄ | Eᵥᵢᵢ | E₄* | Eₑ |
| 3916 | E_{1b}E₂E₃EᵢE₄ | Eᵥᵢᵢ | E₄* | Eₑ |
| 3917 | E₁ₐE₂E₃EᵢᵢE₄ | Eᵥᵢᵢ | E₄* | Eₑ |
| 3918 | E_{1b}E₂E₃EᵢᵢE₄ | Eᵥᵢᵢ | E₄* | Eₑ |
| 3919 | E₁ₐE₂E₃EᵢᵢE_{iib}E₄ | Eᵥᵢᵢ | E₄* | Eₑ |
| 3920 | E_{1b}E₂E₃EᵢᵢE_{iib}E₄ | Eᵥᵢᵢ | E₄* | Eₑ |
| 3921 | E₁ₐE₂Eᵢᵢᵢ | Eᵥᵢᵢ | E₄* | Eₑ |
| 3922 | E_{1b}E₂Eᵢᵢᵢ | Eᵥᵢᵢ | E₄* | Eₑ |
| 3923 | E₁ₐE₂E₃Eᵢᵢᵢ | Eᵥᵢᵢ | E₄* | Eₑ |
| 3924 | E_{1b}E₂E₃Eᵢᵢᵢ | Eᵥᵢᵢ | E₄* | Eₑ |
| 3925 | E₁ₐE₂EᵢᵢᵢE₄ | Eᵥᵢᵢ | E₄* | Eₑ |
| 3926 | E_{1b}E₂EᵢᵢᵢE₄ | Eᵥᵢᵢ | E₄* | Eₑ |
| 3927 | E₁ₐE₂E₃EᵢᵢᵢE₄ | Eᵥᵢᵢ | E₄* | Eₑ |
| 3928 | E_{1b}E₂E₃EᵢᵢᵢE₄ | Eᵥᵢᵢ | E₄* | Eₑ |
| 3929 | E₁ₐE₂Eᵢᵥ | Eᵥᵢᵢ | E₄* | Eₑ |
| 3930 | E_{1b}E₂Eᵢᵥ | Eᵥᵢᵢ | E₄* | Eₑ |
| 3931 | E₁ₐE₂E₃Eᵢᵥ | Eᵥᵢᵢ | E₄* | Eₑ |
| 3932 | E_{1b}E₂E₃Eᵢᵥ | Eᵥᵢᵢ | E₄* | Eₑ |
| 3933 | E₁ₐE₂EᵢᵥE₄ | Eᵥᵢᵢ | E₄* | Eₑ |
| 3934 | E_{1b}E₂EᵢᵥE₄ | Eᵥᵢᵢ | E₄* | Eₑ |
| 3935 | E₁ₐE₂E₃EᵢᵥE₄ | Eᵥᵢᵢ | E₄* | Eₑ |
| 3936 | E_{1b}E₂E₃EᵢᵥE₄ | Eᵥᵢᵢ | E₄* | Eₑ |
| 3937 | E₁ₐE₂ | Eᵥᵢᵢ | E₅ | Eₑ |
| 3938 | E_{1b}E₂ | Eᵥᵢᵢ | E₅ | Eₑ |
| 3939 | E₁ₐE₂E₃ | Eᵥᵢᵢ | E₅ | Eₑ |
| 3940 | E_{1b}E₂E₃ | Eᵥᵢᵢ | E₅ | Eₑ |
| 3941 | E₁ₐE₂Eᵢ | Eᵥᵢᵢ | E₅ | Eₑ |
| 3942 | E_{1b}E₂Eᵢ | Eᵥᵢᵢ | E₅ | Eₑ |
| 3943 | E₁ₐE₂Eᵢᵢ | Eᵥᵢᵢ | E₅ | Eₑ |
| 3944 | E_{1b}E₂Eᵢᵢ | Eᵥᵢᵢ | E₅ | Eₑ |
| 3945 | E₁ₐE₂EᵢᵢE_{iib} | Eᵥᵢᵢ | E₅ | Eₑ |
| 3946 | E_{1b}E₂EᵢᵢE_{iib} | Eᵥᵢᵢ | E₅ | Eₑ |
| 3947 | E₁ₐE₂E₃Eᵢ | Eᵥᵢᵢ | E₅ | Eₑ |
| 3948 | E_{1b}E₂E₃Eᵢ | Eᵥᵢᵢ | E₅ | Eₑ |
| 3949 | E₁ₐE₂E₃Eᵢᵢ | Eᵥᵢᵢ | E₅ | Eₑ |
| 3950 | E_{1b}E₂E₃Eᵢᵢ | Eᵥᵢᵢ | E₅ | Eₑ |
| 3951 | E₁ₐE₂E₃EᵢᵢE_{iib} | Eᵥᵢᵢ | E₅ | Eₑ |
| 3952 | E_{1b}E₂E₃EᵢᵢE_{iib} | Eᵥᵢᵢ | E₅ | Eₑ |
| 3953 | E₁ₐE₂E₄ | Eᵥᵢᵢ | E₅ | Eₑ |
| 3954 | E_{1b}E₂E₄ | Eᵥᵢᵢ | E₅ | Eₑ |
| 3955 | E₁ₐE₂E₃E₄ | Eᵥᵢᵢ | E₅ | Eₑ |
| 3956 | E_{1b}E₂E₃E₄ | Eᵥᵢᵢ | E₅ | Eₑ |
| 3957 | E₁ₐE₂EᵢE₄ | Eᵥᵢᵢ | E₅ | Eₑ |
| 3958 | E_{1b}E₂EᵢE₄ | Eᵥᵢᵢ | E₅ | Eₑ |
| 3959 | E₁ₐE₂EᵢᵢE₄ | Eᵥᵢᵢ | E₅ | Eₑ |
| 3960 | E_{1b}E₂EᵢᵢE₄ | Eᵥᵢᵢ | E₅ | Eₑ |
| 3961 | E₁ₐE₂EᵢᵢE_{iib}E₄ | Eᵥᵢᵢ | E₅ | Eₑ |
| 3962 | E_{1b}E₂EᵢᵢE_{iib}E₄ | Eᵥᵢᵢ | E₅ | Eₑ |
| 3963 | E₁ₐE₂E₃EᵢE₄ | Eᵥᵢᵢ | E₅ | Eₑ |
| 3964 | E_{1b}E₂E₃EᵢE₄ | Eᵥᵢᵢ | E₅ | Eₑ |
| 3965 | E₁ₐE₂E₃EᵢᵢE₄ | Eᵥᵢᵢ | E₅ | Eₑ |
| 3966 | E_{1b}E₂E₃EᵢᵢE₄ | Eᵥᵢᵢ | E₅ | Eₑ |
| 3967 | E₁ₐE₂E₃EᵢᵢE_{iib}E₄ | Eᵥᵢᵢ | E₅ | Eₑ |
| 3968 | E_{1b}E₂E₃EᵢᵢE_{iib}E₄ | Eᵥᵢᵢ | E₅ | Eₑ |
| 3969 | E₁ₐE₂Eᵢᵢᵢ | Eᵥᵢᵢ | E₅ | Eₑ |
| 3970 | E_{1b}E₂Eᵢᵢᵢ | Eᵥᵢᵢ | E₅ | Eₑ |
| 3971 | E₁ₐE₂E₃Eᵢᵢᵢ | Eᵥᵢᵢ | E₅ | Eₑ |
| 3972 | E_{1b}E₂E₃Eᵢᵢᵢ | Eᵥᵢᵢ | E₅ | Eₑ |
| 3973 | E₁ₐE₂EᵢᵢᵢE₄ | Eᵥᵢᵢ | E₅ | Eₑ |
| 3974 | E_{1b}E₂EᵢᵢᵢE₄ | Eᵥᵢᵢ | E₅ | Eₑ |
| 3975 | E₁ₐE₂E₃EᵢᵢᵢE₄ | Eᵥᵢᵢ | E₅ | Eₑ |
| 3976 | E_{1b}E₂E₃EᵢᵢᵢE₄ | Eᵥᵢᵢ | E₅ | Eₑ |
| 3977 | E₁ₐE₂Eᵢᵥ | Eᵥᵢᵢ | E₅ | Eₑ |
| 3978 | E_{1b}E₂Eᵢᵥ | Eᵥᵢᵢ | E₅ | Eₑ |
| 3979 | E₁ₐE₂E₃Eᵢᵥ | Eᵥᵢᵢ | E₅ | Eₑ |
| 3980 | E_{1b}E₂E₃Eᵢᵥ | Eᵥᵢᵢ | E₅ | Eₑ |
| 3981 | E₁ₐE₂EᵢᵥE₄ | Eᵥᵢᵢ | E₅ | Eₑ |
| 3982 | E_{1b}E₂EᵢᵥE₄ | Eᵥᵢᵢ | E₅ | Eₑ |
| 3983 | E₁ₐE₂E₃EᵢᵥE₄ | Eᵥᵢᵢ | E₅ | Eₑ |
| 3984 | E_{1b}E₂E₃EᵢᵥE₄ | Eᵥᵢᵢ | E₅ | Eₑ |
| 3985 | E₁ₐE₂ | Eᵥᵢᵢ | E₄*E₅ | Eₑ |
| 3986 | E_{1b}E₂ | Eᵥᵢᵢ | E₄*E₅ | Eₑ |
| 3987 | E₁ₐE₂E₃ | Eᵥᵢᵢ | E₄*E₅ | Eₑ |
| 3988 | E_{1b}E₂E₃ | Eᵥᵢᵢ | E₄*E₅ | Eₑ |
| 3989 | E₁ₐE₂Eᵢ | Eᵥᵢᵢ | E₄*E₅ | Eₑ |
| 3990 | E_{1b}E₂Eᵢ | Eᵥᵢᵢ | E₄*E₅ | Eₑ |
| 3991 | E₁ₐE₂Eᵢᵢ | Eᵥᵢᵢ | E₄*E₅ | Eₑ |
| 3992 | E_{1b}E₂Eᵢᵢ | Eᵥᵢᵢ | E₄*E₅ | Eₑ |
| 3993 | E₁ₐE₂EᵢᵢE_{iib} | Eᵥᵢᵢ | E₄*E₅ | Eₑ |
| 3994 | E_{1b}E₂EᵢᵢE_{iib} | Eᵥᵢᵢ | E₄*E₅ | Eₑ |
| 3995 | E₁ₐE₂E₃Eᵢ | Eᵥᵢᵢ | E₄*E₅ | Eₑ |
| 3996 | E_{1b}E₂E₃Eᵢ | Eᵥᵢᵢ | E₄*E₅ | Eₑ |
| 3997 | E₁ₐE₂E₃Eᵢᵢ | Eᵥᵢᵢ | E₄*E₅ | Eₑ |
| 3998 | E_{1b}E₂E₃Eᵢᵢ | Eᵥᵢᵢ | E₄*E₅ | Eₑ |
| 3999 | E₁ₐE₂E₃EᵢᵢE_{iib} | Eᵥᵢᵢ | E₄*E₅ | Eₑ |
| 4000 | E_{1b}E₂E₃EᵢᵢE_{iib} | Eᵥᵢᵢ | E₄*E₅ | Eₑ |
| 4001 | E₁ₐE₂E₄ | Eᵥᵢᵢ | E₄*E₅ | Eₑ |
| 4002 | E_{1b}E₂E₄ | Eᵥᵢᵢ | E₄*E₅ | Eₑ |
| 4003 | E₁ₐE₂E₃E₄ | Eᵥᵢᵢ | E₄*E₅ | Eₑ |
| 4004 | E_{1b}E₂E₃E₄ | Eᵥᵢᵢ | E₄*E₅ | Eₑ |
| 4005 | E₁ₐE₂EᵢE₄ | Eᵥᵢᵢ | E₄*E₅ | Eₑ |
| 4006 | E_{1b}E₂EᵢE₄ | Eᵥᵢᵢ | E₄*E₅ | Eₑ |
| 4007 | E₁ₐE₂EᵢᵢE₄ | Eᵥᵢᵢ | E₄*E₅ | Eₑ |
| 4008 | E_{1b}E₂EᵢᵢE₄ | Eᵥᵢᵢ | E₄*E₅ | Eₑ |
| 4009 | E₁ₐE₂EᵢᵢE_{iib}E₄ | Eᵥᵢᵢ | E₄*E₅ | Eₑ |
| 4010 | E_{1b}E₂EᵢᵢE_{iib}E₄ | Eᵥᵢᵢ | E₄*E₅ | Eₑ |
| 4011 | E₁ₐE₂E₃EᵢE₄ | Eᵥᵢᵢ | E₄*E₅ | Eₑ |
| 4012 | E_{1b}E₂E₃EᵢE₄ | Eᵥᵢᵢ | E₄*E₅ | Eₑ |
| 4013 | E₁ₐE₂E₃EᵢᵢE₄ | Eᵥᵢᵢ | E₄*E₅ | Eₑ |
| 4014 | E_{1b}E₂E₃EᵢᵢE₄ | Eᵥᵢᵢ | E₄*E₅ | Eₑ |
| 4015 | E₁ₐE₂E₃EᵢᵢE_{iib}E₄ | Eᵥᵢᵢ | E₄*E₅ | Eₑ |
| 4016 | E_{1b}E₂E₃EᵢᵢE_{iib}E₄ | Eᵥᵢᵢ | E₄*E₅ | Eₑ |
| 4017 | E₁ₐE₂Eᵢᵢᵢ | Eᵥᵢᵢ | E₄*E₅ | Eₑ |
| 4018 | E_{1b}E₂Eᵢᵢᵢ | Eᵥᵢᵢ | E₄*E₅ | Eₑ |
| 4019 | E₁ₐE₂E₃Eᵢᵢᵢ | Eᵥᵢᵢ | E₄*E₅ | Eₑ |
| 4020 | E_{1b}E₂E₃Eᵢᵢᵢ | Eᵥᵢᵢ | E₄*E₅ | Eₑ |
| 4021 | E₁ₐE₂EᵢᵢᵢE₄ | Eᵥᵢᵢ | E₄*E₅ | Eₑ |
| 4022 | E_{1b}E₂EᵢᵢᵢE₄ | Eᵥᵢᵢ | E₄*E₅ | Eₑ |
| 4023 | E₁ₐE₂E₃EᵢᵢᵢE₄ | Eᵥᵢᵢ | E₄*E₅ | Eₑ |
| 4024 | E_{1b}E₂E₃EᵢᵢᵢE₄ | Eᵥᵢᵢ | E₄*E₅ | Eₑ |
| 4025 | E₁ₐE₂Eᵢᵥ | Eᵥᵢᵢ | E₄*E₅ | Eₑ |
| 4026 | E_{1b}E₂Eᵢᵥ | Eᵥᵢᵢ | E₄*E₅ | Eₑ |
| 4027 | E₁ₐE₂E₃Eᵢᵥ | Eᵥᵢᵢ | E₄*E₅ | Eₑ |
| 4028 | E_{1b}E₂E₃Eᵢᵥ | Eᵥᵢᵢ | E₄*E₅ | Eₑ |
| 4029 | E₁ₐE₂EᵢᵥE₄ | Eᵥᵢᵢ | E₄*E₅ | Eₑ |
| 4030 | E_{1b}E₂EᵢᵥE₄ | Eᵥᵢᵢ | E₄*E₅ | Eₑ |
| 4031 | E₁ₐE₂E₃EᵢᵥE₄ | Eᵥᵢᵢ | E₄*E₅ | Eₑ |
| 4032 | E_{1b}E₂E₃EᵢᵥE₄ | Eᵥᵢᵢ | E₄*E₅ | Eₑ |
| 4033 | E₁ₐE₂ | AlkLEᵥᵢᵢ | | Eₑ |
| 4034 | E_{1b}E₂ | AlkLEᵥᵢᵢ | | Eₑ |
| 4035 | E₁ₐE₂E₃ | AlkLEᵥᵢᵢ | | Eₑ |
| 4036 | E_{1b}E₂E₃ | AlkLEᵥᵢᵢ | | Eₑ |
| 4037 | E₁ₐE₂Eᵢ | AlkLEᵥᵢᵢ | | Eₑ |
| 4038 | E_{1b}E₂Eᵢ | AlkLEᵥᵢᵢ | | Eₑ |
| 4039 | E₁ₐE₂Eᵢᵢ | AlkLEᵥᵢᵢ | | Eₑ |
| 4040 | E_{1b}E₂Eᵢᵢ | AlkLEᵥᵢᵢ | | Eₑ |
| 4041 | E₁ₐE₂EᵢᵢE_{iib} | AlkLEᵥᵢᵢ | | Eₑ |
| 4042 | E_{1b}E₂EᵢᵢE_{iib} | AlkLEᵥᵢᵢ | | Eₑ |
| 4043 | E₁ₐE₂E₃Eᵢ | AlkLEᵥᵢᵢ | | Eₑ |
| 4044 | E_{1b}E₂E₃Eᵢ | AlkLEᵥᵢᵢ | | Eₑ |
| 4045 | E₁ₐE₂E₃Eᵢᵢ | AlkLEᵥᵢᵢ | | Eₑ |
| 4046 | E_{1b}E₂E₃Eᵢᵢ | AlkLEᵥᵢᵢ | | Eₑ |
| 4047 | E₁ₐE₂E₃EᵢᵢE_{iib} | AlkLEᵥᵢᵢ | | Eₑ |
| 4048 | E_{1b}E₂E₃EᵢᵢE_{iib} | AlkLEᵥᵢᵢ | | Eₑ |
| 4049 | E₁ₐE₂E₄ | AlkLEᵥᵢᵢ | | Eₑ |
| 4050 | E_{1b}E₂E₄ | AlkLEᵥᵢᵢ | | Eₑ |
| 4051 | E₁ₐE₂E₃E₄ | AlkLEᵥᵢᵢ | | Eₑ |
| 4052 | E_{1b}E₂E₃E₄ | AlkLEᵥᵢᵢ | | Eₑ |
| 4053 | E₁ₐE₂EᵢE₄ | AlkLEᵥᵢᵢ | | Eₑ |
| 4054 | E_{1b}E₂EᵢE₄ | AlkLEᵥᵢᵢ | | Eₑ |
| 4055 | E₁ₐE₂EᵢᵢE₄ | AlkLEᵥᵢᵢ | | Eₑ |
| 4056 | E_{1b}E₂EᵢᵢE₄ | AlkLEᵥᵢᵢ | | Eₑ |
| 4057 | E₁ₐE₂EᵢᵢE_{iib}E₄ | AlkLEᵥᵢᵢ | | Eₑ |
| 4058 | E_{1b}E₂EᵢᵢE_{iib}E₄ | AlkLEᵥᵢᵢ | | Eₑ |
| 4059 | E₁ₐE₂E₃EᵢE₄ | AlkLEᵥᵢᵢ | | Eₑ |
| 4060 | E_{1b}E₂E₃EᵢE₄ | AlkLEᵥᵢᵢ | | Eₑ |
| 4061 | E₁ₐE₂E₃EᵢᵢE₄ | AlkLEᵥᵢᵢ | | Eₑ |
| 4062 | E_{1b}E₂E₃EᵢᵢE₄ | AlkLEᵥᵢᵢ | | Eₑ |
| 4063 | E₁ₐE₂E₃EᵢᵢE_{iib}E₄ | AlkLEᵥᵢᵢ | | Eₑ |
| 4064 | E_{1b}E₂E₃EᵢᵢE_{iib}E₄ | AlkLEᵥᵢᵢ | | Eₑ |
| 4065 | E₁ₐE₂Eᵢᵢᵢ | AlkLEᵥᵢᵢ | | Eₑ |
| 4066 | E_{1b}E₂Eᵢᵢᵢ | AlkLEᵥᵢᵢ | | Eₑ |
| 4067 | E₁ₐE₂E₃Eᵢᵢᵢ | AlkLEᵥᵢᵢ | | Eₑ |
| 4068 | E_{1b}E₂E₃Eᵢᵢᵢ | AlkLEᵥᵢᵢ | | Eₑ |
| 4069 | E₁ₐE₂EᵢᵢᵢE₄ | AlkLEᵥᵢᵢ | | Eₑ |
| 4070 | E_{1b}E₂EᵢᵢᵢE₄ | AlkLEᵥᵢᵢ | | Eₑ |
| 4071 | E₁ₐE₂E₃EᵢᵢᵢE₄ | AlkLEᵥᵢᵢ | | Eₑ |
| 4072 | E_{1b}E₂E₃EᵢᵢᵢE₄ | AlkLEᵥᵢᵢ | | Eₑ |
| 4073 | E₁ₐE₂Eᵢᵥ | AlkLEᵥᵢᵢ | | Eₑ |
| 4074 | E_{1b}E₂Eᵢᵥ | AlkLEᵥᵢᵢ | | Eₑ |
| 4075 | E₁ₐE₂E₃Eᵢᵥ | AlkLEᵥᵢᵢ | | Eₑ |
| 4076 | E_{1b}E₂E₃Eᵢᵥ | AlkLEᵥᵢᵢ | | Eₑ |
| 4077 | E₁ₐE₂EᵢᵥE₄ | AlkLEᵥᵢᵢ | | Eₑ |
| 4078 | E_{1b}E₂EᵢᵥE₄ | AlkLEᵥᵢᵢ | | Eₑ |
| 4079 | E₁ₐE₂E₃EᵢᵥE₄ | AlkLEᵥᵢᵢ | | Eₑ |
| 4080 | E_{1b}E₂E₃EᵢᵥE₄ | AlkLEᵥᵢᵢ | | Eₑ |
| 4081 | E₁ₐE₂ | AlkLEᵥᵢᵢ | E₄* | Eₑ |
| 4082 | E_{1b}E₂ | AlkLEᵥᵢᵢ | E₄* | Eₑ |
| 4083 | E₁ₐE₂E₃ | AlkLEᵥᵢᵢ | E₄* | Eₑ |
| 4084 | E_{1b}E₂E₃ | AlkLEᵥᵢᵢ | E₄* | Eₑ |
| 4085 | E₁ₐE₂Eᵢ | AlkLEᵥᵢᵢ | E₄* | Eₑ |
| 4086 | E_{1b}E₂Eᵢ | AlkLEᵥᵢᵢ | E₄* | Eₑ |
| 4087 | E₁ₐE₂Eᵢᵢ | AlkLEᵥᵢᵢ | E₄* | Eₑ |
| 4088 | E_{1b}E₂Eᵢᵢ | AlkLEᵥᵢᵢ | E₄* | Eₑ |
| 4089 | E₁ₐE₂EᵢᵢE_{iib} | AlkLEᵥᵢᵢ | E₄* | Eₑ |
| 4090 | E_{1b}E₂EᵢᵢE_{iib} | AlkLEᵥᵢᵢ | E₄* | Eₑ |
| 4091 | E₁ₐE₂E₃Eᵢ | AlkLEᵥᵢᵢ | E₄* | Eₑ |
| 4092 | E_{1b}E₂E₃Eᵢ | AlkLEᵥᵢᵢ | E₄* | Eₑ |
| 4093 | E₁ₐE₂E₃Eᵢᵢ | AlkLEᵥᵢᵢ | E₄* | Eₑ |
| 4094 | E_{1b}E₂E₃Eᵢᵢ | AlkLEᵥᵢᵢ | E₄* | Eₑ |
| 4095 | E₁ₐE₂E₃EᵢᵢE_{iib} | AlkLEᵥᵢᵢ | E₄* | Eₑ |
| 4096 | E_{1b}E₂E₃EᵢᵢE_{iib} | AlkLEᵥᵢᵢ | E₄* | Eₑ |
| 4097 | E₁ₐE₂E₄ | AlkLEᵥᵢᵢ | E₄* | Eₑ |
| 4098 | E_{1b}E₂E₄ | AlkLEᵥᵢᵢ | E₄* | Eₑ |
| 4099 | E₁ₐE₂E₃E₄ | AlkLEᵥᵢᵢ | E₄* | Eₑ |
| 4100 | E_{1b}E₂E₃E₄ | AlkLEᵥᵢᵢ | E₄* | Eₑ |
| 4101 | E₁ₐE₂EᵢE₄ | AlkLEᵥᵢᵢ | E₄* | Eₑ |
| 4102 | E_{1b}E₂EᵢE₄ | AlkLEᵥᵢᵢ | E₄* | Eₑ |
| 4103 | E₁ₐE₂EᵢᵢE₄ | AlkLEᵥᵢᵢ | E₄* | Eₑ |
| 4104 | E_{1b}E₂EᵢᵢE₄ | AlkLEᵥᵢᵢ | E₄* | Eₑ |
| 4105 | E₁ₐE₂EᵢᵢE_{iib}E₄ | AlkLEᵥᵢᵢ | E₄* | Eₑ |
| 4106 | E_{1b}E₂EᵢᵢE_{iib}E₄ | AlkLEᵥᵢᵢ | E₄* | Eₑ |
| 4107 | E₁ₐE₂E₃EᵢE₄ | AlkLEᵥᵢᵢ | E₄* | Eₑ |
| 4108 | E_{1b}E₂E₃EᵢE₄ | AlkLEᵥᵢᵢ | E₄* | Eₑ |
| 4109 | E₁ₐE₂E₃EᵢᵢE₄ | AlkLEᵥᵢᵢ | E₄* | Eₑ |
| 4110 | E_{1b}E₂E₃EᵢᵢE₄ | AlkLEᵥᵢᵢ | E₄* | Eₑ |
| 4111 | E₁ₐE₂E₃EᵢᵢE_{iib}E₄ | AlkLEᵥᵢᵢ | E₄* | Eₑ |
| 4112 | E_{1b}E₂E₃EᵢᵢE_{iib}E₄ | AlkLEᵥᵢᵢ | E₄* | Eₑ |
| 4113 | E₁ₐE₂Eᵢᵢᵢ | AlkLEᵥᵢᵢ | E₄* | Eₑ |
| 4114 | E_{1b}E₂Eᵢᵢᵢ | AlkLEᵥᵢᵢ | E₄* | Eₑ |
| 4115 | E₁ₐE₂E₃Eᵢᵢᵢ | AlkLEᵥᵢᵢ | E₄* | Eₑ |
| 4116 | E_{1b}E₂E₃Eᵢᵢᵢ | AlkLEᵥᵢᵢ | E₄* | Eₑ |
| 4117 | E₁ₐE₂EᵢᵢᵢE₄ | AlkLEᵥᵢᵢ | E₄* | Eₑ |
| 4118 | E_{1b}E₂EᵢᵢᵢE₄ | AlkLEᵥᵢᵢ | E₄* | Eₑ |
| 4119 | E₁ₐE₂E₃EᵢᵢᵢE₄ | AlkLEᵥᵢᵢ | E₄* | Eₑ |
| 4120 | E_{1b}E₂E₃EᵢᵢᵢE₄ | AlkLEᵥᵢᵢ | E₄* | Eₑ |
| 4121 | E₁ₐE₂Eᵢᵥ | AlkLEᵥᵢᵢ | E₄* | Eₑ |
| 4122 | E_{1b}E₂Eᵢᵥ | AlkLEᵥᵢᵢ | E₄* | Eₑ |
| 4123 | E₁ₐE₂E₃Eᵢᵥ | AlkLEᵥᵢᵢ | E₄* | Eₑ |
| 4124 | E_{1b}E₂E₃Eᵢᵥ | AlkLEᵥᵢᵢ | E₄* | Eₑ |
| 4125 | E₁ₐE₂EᵢᵥE₄ | AlkLEᵥᵢᵢ | E₄* | Eₑ |
| 4126 | E_{1b}E₂EᵢᵥE₄ | AlkLEᵥᵢᵢ | E₄* | Eₑ |
| 4127 | E₁ₐE₂E₃EᵢᵥE₄ | AlkLEᵥᵢᵢ | E₄* | Eₑ |
| 4128 | E_{1b}E₂E₃EᵢᵥE₄ | AlkLEᵥᵢᵢ | E₄* | Eₑ |
| 4129 | E₁ₐE₂ | AlkLEᵥᵢᵢ | E₅ | Eₑ |
| 4130 | E_{1b}E₂ | AlkLEᵥᵢᵢ | E₅ | Eₑ |
| 4131 | E₁ₐE₂E₃ | AlkLEᵥᵢᵢ | E₅ | Eₑ |
| 4132 | E_{1b}E₂E₃ | AlkLEᵥᵢᵢ | E₅ | Eₑ |
| 4133 | E₁ₐE₂Eᵢ | AlkLEᵥᵢᵢ | E₅ | Eₑ |
| 4134 | E_{1b}E₂Eᵢ | AlkLEᵥᵢᵢ | E₅ | Eₑ |
| 4135 | E₁ₐE₂Eᵢᵢ | AlkLEᵥᵢᵢ | E₅ | Eₑ |
| 4136 | E_{1b}E₂Eᵢᵢ | AlkLEᵥᵢᵢ | E₅ | Eₑ |
| 4137 | E₁ₐE₂EᵢᵢE_{iib} | AlkLEᵥᵢᵢ | E₅ | Eₑ |
| 4138 | E_{1b}E₂EᵢᵢE_{iib} | AlkLEᵥᵢᵢ | E₅ | Eₑ |
| 4139 | E₁ₐE₂E₃Eᵢ | AlkLEᵥᵢᵢ | E₅ | Eₑ |
| 4140 | E_{1b}E₂E₃Eᵢ | AlkLEᵥᵢᵢ | E₅ | Eₑ |
| 4141 | E₁ₐE₂E₃Eᵢᵢ | AlkLEᵥᵢᵢ | E₅ | Eₑ |
| 4142 | E_{1b}E₂E₃Eᵢᵢ | AlkLEᵥᵢᵢ | E₅ | Eₑ |
| 4143 | E₁ₐE₂E₃EᵢᵢE_{iib} | AlkLEᵥᵢᵢ | E₅ | Eₑ |
| 4144 | E_{1b}E₂E₃EᵢᵢE_{iib} | AlkLEᵥᵢᵢ | E₅ | Eₑ |
| 4145 | E₁ₐE₂E₄ | AlkLEᵥᵢᵢ | E₅ | Eₑ |
| 4146 | E_{1b}E₂E₄ | AlkLEᵥᵢᵢ | E₅ | Eₑ |
| 4147 | E₁ₐE₂E₃E₄ | AlkLEᵥᵢᵢ | E₅ | Eₑ |
| 4148 | E_{1b}E₂E₃E₄ | AlkLEᵥᵢᵢ | E₅ | Eₑ |
| 4149 | E₁ₐE₂EᵢE₄ | AlkLEᵥᵢᵢ | E₅ | Eₑ |
| 4150 | E_{1b}E₂EᵢE₄ | AlkLEᵥᵢᵢ | E₅ | Eₑ |
| 4151 | E₁ₐE₂EᵢᵢE₄ | AlkLEᵥᵢᵢ | E₅ | Eₑ |
| 4152 | E_{1b}E₂EᵢᵢE₄ | AlkLEᵥᵢᵢ | E₅ | Eₑ |
| 4153 | E₁ₐE₂EᵢᵢE_{iib}E₄ | AlkLEᵥᵢᵢ | E₅ | Eₑ |
| 4154 | E_{1b}E₂EᵢᵢE_{iib}E₄ | AlkLEᵥᵢᵢ | E₅ | Eₑ |
| 4155 | E₁ₐE₂E₃EᵢE₄ | AlkLEᵥᵢᵢ | E₅ | Eₑ |
| 4156 | E_{1b}E₂E₃EᵢE₄ | AlkLEᵥᵢᵢ | E₅ | Eₑ |
| 4157 | E₁ₐE₂E₃EᵢᵢE₄ | AlkLEᵥᵢᵢ | E₅ | Eₑ |
| 4158 | E_{1b}E₂E₃EᵢᵢE₄ | AlkLEᵥᵢᵢ | E₅ | Eₑ |
| 4159 | E₁ₐE₂E₃EᵢᵢE_{iib}E₄ | AlkLEᵥᵢᵢ | E₅ | Eₑ |
| 4160 | E_{1b}E₂E₃EᵢᵢE_{iib}E₄ | AlkLEᵥᵢᵢ | E₅ | Eₑ |
| 4161 | E₁ₐE₂Eᵢᵢᵢ | AlkLEᵥᵢᵢ | E₅ | Eₑ |
| 4162 | E_{1b}E₂Eᵢᵢᵢ | AlkLEᵥᵢᵢ | E₅ | Eₑ |
| 4163 | E₁ₐE₂E₃Eᵢᵢᵢ | AlkLEᵥᵢᵢ | E₅ | Eₑ |
| 4164 | E_{1b}E₂E₃Eᵢᵢᵢ | AlkLEᵥᵢᵢ | E₅ | Eₑ |
| 4165 | E₁ₐE₂EᵢᵢᵢE₄ | AlkLEᵥᵢᵢ | E₅ | Eₑ |
| 4166 | E_{1b}E₂EᵢᵢᵢE₄ | AlkLEᵥᵢᵢ | E₅ | Eₑ |
| 4167 | E₁ₐE₂E₃EᵢᵢᵢE₄ | AlkLEᵥᵢᵢ | E₅ | Eₑ |
| 4168 | E_{1b}E₂E₃EᵢᵢᵢE₄ | AlkLEᵥᵢᵢ | E₅ | Eₑ |
| 4169 | E₁ₐE₂Eᵢᵥ | AlkLEᵥᵢᵢ | E₅ | Eₑ |
| 4170 | E_{1b}E₂Eᵢᵥ | AlkLEᵥᵢᵢ | E₅ | Eₑ |
| 4171 | E₁ₐE₂E₃Eᵢᵥ | AlkLEᵥᵢᵢ | E₅ | Eₑ |
| 4172 | E_{1b}E₂E₃Eᵢᵥ | AlkLEᵥᵢᵢ | E₅ | Eₑ |
| 4173 | E₁ₐE₂EᵢᵥE₄ | AlkLEᵥᵢᵢ | E₅ | Eₑ |
| 4174 | E_{1b}E₂EᵢᵥE₄ | AlkLEᵥᵢᵢ | E₅ | Eₑ |
| 4175 | E₁ₐE₂E₃EᵢᵥE₄ | AlkLEᵥᵢᵢ | E₅ | Eₑ |
| 4176 | E_{1b}E₂E₃EᵢᵥE₄ | AlkLEᵥᵢᵢ | E₅ | Eₑ |
| 4177 | E₁ₐE₂ | AlkLEᵥᵢᵢ | E₄*E₅ | Eₑ |
| 4178 | E_{1b}E₂ | AlkLEᵥᵢᵢ | E₄*E₅ | Eₑ |
| 4179 | E₁ₐE₂E₃ | AlkLEᵥᵢᵢ | E₄*E₅ | Eₑ |
| 4180 | E_{1b}E₂E₃ | AlkLEᵥᵢᵢ | E₄*E₅ | Eₑ |
| 4181 | E₁ₐE₂Eᵢ | AlkLEᵥᵢᵢ | E₄*E₅ | Eₑ |
| 4182 | E_{1b}E₂Eᵢ | AlkLEᵥᵢᵢ | E₄*E₅ | Eₑ |
| 4183 | E₁ₐE₂Eᵢᵢ | AlkLEᵥᵢᵢ | E₄*E₅ | Eₑ |
| 4184 | E_{1b}E₂Eᵢᵢ | AlkLEᵥᵢᵢ | E₄*E₅ | Eₑ |
| 4185 | E₁ₐE₂EᵢᵢE_{iib} | AlkLEᵥᵢᵢ | E₄*E₅ | Eₑ |
| 4186 | E_{1b}E₂EᵢᵢE_{iib} | AlkLEᵥᵢᵢ | E₄*E₅ | Eₑ |
| 4187 | E₁ₐE₂E₃Eᵢ | AlkLEᵥᵢᵢ | E₄*E₅ | Eₑ |
| 4188 | E_{1b}E₂E₃Eᵢ | AlkLEᵥᵢᵢ | E₄*E₅ | Eₑ |
| 4189 | E₁ₐE₂E₃Eᵢᵢ | AlkLEᵥᵢᵢ | E₄*E₅ | Eₑ |
| 4190 | E_{1b}E₂E₃Eᵢᵢ | AlkLEᵥᵢᵢ | E₄*E₅ | Eₑ |
| 4191 | E₁ₐE₂E₃EᵢᵢE_{iib} | AlkLEᵥᵢᵢ | E₄*E₅ | Eₑ |
| 4192 | E_{1b}E₂E₃EᵢᵢE_{iib} | AlkLEᵥᵢᵢ | E₄*E₅ | Eₑ |
| 4193 | E₁ₐE₂E₄ | AlkLEᵥᵢᵢ | E₄*E₅ | Eₑ |
| 4194 | E_{1b}E₂E₄ | AlkLEᵥᵢᵢ | E₄*E₅ | Eₑ |
| 4195 | E₁ₐE₂E₃E₄ | AlkLEᵥᵢᵢ | E₄*E₅ | Eₑ |
| 4196 | E_{1b}E₂E₃E₄ | AlkLEᵥᵢᵢ | E₄*E₅ | Eₑ |
| 4197 | E₁ₐE₂EᵢE₄ | AlkLEᵥᵢᵢ | E₄*E₅ | Eₑ |
| 4198 | E_{1b}E₂EᵢE₄ | AlkLEᵥᵢᵢ | E₄*E₅ | Eₑ |
| 4199 | E₁ₐE₂EᵢᵢE₄ | AlkLEᵥᵢᵢ | E₄*E₅ | Eₑ |
| 4200 | E_{1b}E₂EᵢᵢE₄ | AlkLEᵥᵢᵢ | E₄*E₅ | Eₑ |
| 4201 | E₁ₐE₂EᵢᵢE_{iib}E₄ | AlkLEᵥᵢᵢ | E₄*E₅ | Eₑ |
| 4202 | E_{1b}E₂EᵢᵢE_{iib}E₄ | AlkLEᵥᵢᵢ | E₄*E₅ | Eₑ |
| 4203 | E₁ₐE₂E₃EᵢE₄ | AlkLEᵥᵢᵢ | E₄*E₅ | Eₑ |
| 4204 | E_{1b}E₂E₃EᵢE₄ | AlkLEᵥᵢᵢ | E₄*E₅ | Eₑ |
| 4205 | E₁ₐE₂E₃EᵢᵢE₄ | AlkLEᵥᵢᵢ | E₄*E₅ | Eₑ |
| 4206 | E_{1b}E₂E₃EᵢᵢE₄ | AlkLEᵥᵢᵢ | E₄*E₅ | Eₑ |
| 4207 | E₁ₐE₂E₃EᵢᵢE_{iib}E₄ | AlkLEᵥᵢᵢ | E₄*E₅ | Eₑ |
| 4208 | E_{1b}E₂E₃EᵢᵢE_{iib}E₄ | AlkLEᵥᵢᵢ | E₄*E₅ | Eₑ |
| 4209 | E₁ₐE₂Eᵢᵢᵢ | AlkLEᵥᵢᵢ | E₄*E₅ | Eₑ |
| 4210 | E_{1b}E₂Eᵢᵢᵢ | AlkLEᵥᵢᵢ | E₄*E₅ | Eₑ |
| 4211 | E₁ₐE₂E₃Eᵢᵢᵢ | AlkLEᵥᵢᵢ | E₄*E₅ | Eₑ |
| 4212 | E_{1b}E₂E₃Eᵢᵢᵢ | AlkLEᵥᵢᵢ | E₄*E₅ | Eₑ |
| 4213 | E₁ₐE₂EᵢᵢᵢE₄ | AlkLEᵥᵢᵢ | E₄*E₅ | Eₑ |
| 4214 | E_{1b}E₂EᵢᵢᵢE₄ | AlkLEᵥᵢᵢ | E₄*E₅ | Eₑ |
| 4215 | E₁ₐE₂E₃EᵢᵢᵢE₄ | AlkLEᵥᵢᵢ | E₄*E₅ | Eₑ |
| 4216 | E_{1b}E₂E₃EᵢᵢᵢE₄ | AlkLEᵥᵢᵢ | E₄*E₅ | Eₑ |
| 4217 | E₁ₐE₂Eᵢᵥ | AlkLEᵥᵢᵢ | E₄*E₅ | Eₑ |
| 4218 | E_{1b}E₂Eᵢᵥ | AlkLEᵥᵢᵢ | E₄*E₅ | Eₑ |
| 4219 | E₁ₐE₂E₃Eᵢᵥ | AlkLEᵥᵢᵢ | E₄*E₅ | Eₑ |
| 4220 | E_{1b}E₂E₃Eᵢᵥ | AlkLEᵥᵢᵢ | E₄*E₅ | Eₑ |
| 4221 | E₁ₐE₂EᵢᵥE₄ | AlkLEᵥᵢᵢ | E₄*E₅ | Eₑ |
| 4222 | E_{1b}E₂EᵢᵥE₄ | AlkLEᵥᵢᵢ | E₄*E₅ | Eₑ |
| 4223 | E₁ₐE₂E₃EᵢᵥE₄ | AlkLEᵥᵢᵢ | E₄*E₅ | Eₑ |
| 4224 | E_{1b}E₂E₃EᵢᵥE₄ | AlkLEᵥᵢᵢ | E₄*E₅ | Eₑ |
| 4225 | E₁ₐE₂ | Eᵥᵢᵢ | | E_{f} |
| 4226 | E_{1b}E₂ | Eᵥᵢᵢ | | E_{f} |
| 4227 | E₁ₐE₂E₃ | Eᵥᵢᵢ | | E_{f} |
| 4228 | E_{1b}E₂E₃ | Eᵥᵢᵢ | | E_{f} |
| 4229 | E₁ₐE₂Eᵢ | Eᵥᵢᵢ | | E_{f} |
| 4230 | E_{1b}E₂Eᵢ | Eᵥᵢᵢ | | E_{f} |
| 4231 | E₁ₐE₂Eᵢᵢ | Eᵥᵢᵢ | | E_{f} |
| 4232 | E_{1b}E₂Eᵢᵢ | Eᵥᵢᵢ | | E_{f} |
| 4233 | E₁ₐE₂EᵢᵢE_{iib} | Eᵥᵢᵢ | | E_{f} |
| 4234 | E_{1b}E₂EᵢᵢE_{iib} | Eᵥᵢᵢ | | E_{f} |
| 4235 | E₁ₐE₂E₃Eᵢ | Eᵥᵢᵢ | | E_{f} |
| 4236 | E_{1b}E₂E₃Eᵢ | Eᵥᵢᵢ | | E_{f} |
| 4237 | E₁ₐE₂E₃Eᵢᵢ | Eᵥᵢᵢ | | E_{f} |
| 4238 | E_{1b}E₂E₃Eᵢᵢ | Eᵥᵢᵢ | | E_{f} |
| 4239 | E₁ₐE₂E₃EᵢᵢE_{iib} | Eᵥᵢᵢ | | E_{f} |
| 4240 | E_{1b}E₂E₃EᵢᵢE_{iib} | Eᵥᵢᵢ | | E_{f} |
| 4241 | E₁ₐE₂E₄ | Eᵥᵢᵢ | | E_{f} |
| 4242 | E_{1b}E₂E₄ | Eᵥᵢᵢ | | E_{f} |
| 4243 | E₁ₐE₂E₃E₄ | Eᵥᵢᵢ | | E_{f} |
| 4244 | E_{1b}E₂E₃E₄ | Eᵥᵢᵢ | | E_{f} |
| 4245 | E₁ₐE₂EᵢE₄ | Eᵥᵢᵢ | | E_{f} |
| 4246 | E_{1b}E₂EᵢE₄ | Eᵥᵢᵢ | | E_{f} |
| 4247 | E₁ₐE₂EᵢᵢE₄ | Eᵥᵢᵢ | | E_{f} |
| 4248 | E_{1b}E₂EᵢᵢE₄ | Eᵥᵢᵢ | | E_{f} |
| 4249 | E₁ₐE₂EᵢᵢE_{iib}E₄ | Eᵥᵢᵢ | | E_{f} |
| 4250 | E_{1b}E₂EᵢᵢE_{iib}E₄ | Eᵥᵢᵢ | | E_{f} |
| 4251 | E₁ₐE₂E₃EᵢE₄ | Eᵥᵢᵢ | | E_{f} |
| 4252 | E_{1b}E₂E₃EᵢE₄ | Eᵥᵢᵢ | | E_{f} |
| 4253 | E₁ₐE₂E₃EᵢᵢE₄ | Eᵥᵢᵢ | | E_{f} |
| 4254 | E_{1b}E₂E₃EᵢᵢE₄ | Eᵥᵢᵢ | | E_{f} |
| 4255 | E₁ₐE₂E₃EᵢᵢE_{iib}E₄ | Eᵥᵢᵢ | | E_{f} |
| 4256 | E_{1b}E₂E₃EᵢᵢE_{iib}E₄ | Eᵥᵢᵢ | | E_{f} |
| 4257 | E₁ₐE₂Eᵢᵢᵢ | Eᵥᵢᵢ | | E_{f} |
| 4258 | E_{1b}E₂Eᵢᵢᵢ | Eᵥᵢᵢ | | E_{f} |
| 4259 | E₁ₐE₂E₃Eᵢᵢᵢ | Eᵥᵢᵢ | | E_{f} |
| 4260 | E_{1b}E₂E₃Eᵢᵢᵢ | Eᵥᵢᵢ | | E_{f} |
| 4261 | E₁ₐE₂EᵢᵢᵢE₄ | Eᵥᵢᵢ | | E_{f} |
| 4262 | E_{1b}E₂EᵢᵢᵢE₄ | Eᵥᵢᵢ | | E_{f} |
| 4263 | E₁ₐE₂E₃EᵢᵢᵢE₄ | Eᵥᵢᵢ | | E_{f} |
| 4264 | E_{1b}E₂E₃EᵢᵢᵢE₄ | Eᵥᵢᵢ | | E_{f} |
| 4265 | E₁ₐE₂Eᵢᵥ | Eᵥᵢᵢ | | E_{f} |
| 4266 | E_{1b}E₂Eᵢᵥ | Eᵥᵢᵢ | | E_{f} |
| 4267 | E₁ₐE₂E₃Eᵢᵥ | Eᵥᵢᵢ | | E_{f} |
| 4268 | E_{1b}E₂E₃Eᵢᵥ | Eᵥᵢᵢ | | E_{f} |
| 4269 | E₁ₐE₂EᵢᵥE₄ | Eᵥᵢᵢ | | E_{f} |
| 4270 | E_{1b}E₂EᵢᵥE₄ | Eᵥᵢᵢ | | E_{f} |
| 4271 | E₁ₐE₂E₃EᵢᵥE₄ | Eᵥᵢᵢ | | E_{f} |
| 4272 | E_{1b}E₂E₃EᵢᵥE₄ | Eᵥᵢᵢ | | E_{f} |
| 4273 | E₁ₐE₂ | Eᵥᵢᵢ | E₄* | E_{f} |
| 4274 | E_{1b}E₂ | Eᵥᵢᵢ | E₄* | E_{f} |
| 4275 | E₁ₐE₂E₃ | Eᵥᵢᵢ | E₄* | E_{f} |
| 4276 | E_{1b}E₂E₃ | Eᵥᵢᵢ | E₄* | E_{f} |
| 4277 | E₁ₐE₂Eᵢ | Eᵥᵢᵢ | E₄* | E_{f} |
| 4278 | E_{1b}E₂Eᵢ | Eᵥᵢᵢ | E₄* | E_{f} |
| 4279 | E₁ₐE₂Eᵢᵢ | Eᵥᵢᵢ | E₄* | E_{f} |
| 4280 | E_{1b}E₂Eᵢᵢ | Eᵥᵢᵢ | E₄* | E_{f} |
| 4281 | E₁ₐE₂EᵢᵢE_{iib} | Eᵥᵢᵢ | E₄* | E_{f} |
| 4282 | E_{1b}E₂EᵢᵢE_{iib} | Eᵥᵢᵢ | E₄* | E_{f} |
| 4283 | E₁ₐE₂E₃Eᵢ | Eᵥᵢᵢ | E₄* | E_{f} |
| 4284 | E_{1b}E₂E₃Eᵢ | Eᵥᵢᵢ | E₄* | E_{f} |
| 4285 | E₁ₐE₂E₃Eᵢᵢ | Eᵥᵢᵢ | E₄* | E_{f} |
| 4286 | E_{1b}E₂E₃Eᵢᵢ | Eᵥᵢᵢ | E₄* | E_{f} |
| 4287 | E₁ₐE₂E₃EᵢᵢE_{iib} | Eᵥᵢᵢ | E₄* | E_{f} |
| 4288 | E_{1b}E₂E₃EᵢᵢE_{iib} | Eᵥᵢᵢ | E₄* | E_{f} |
| 4289 | E₁ₐE₂E₄ | Eᵥᵢᵢ | E₄* | E_{f} |
| 4290 | E_{1b}E₂E₄ | Eᵥᵢᵢ | E₄* | E_{f} |
| 4291 | E₁ₐE₂E₃E₄ | Eᵥᵢᵢ | E₄* | E_{f} |
| 4292 | E_{1b}E₂E₃E₄ | Eᵥᵢᵢ | E₄* | E_{f} |
| 4293 | E₁ₐE₂EᵢE₄ | Eᵥᵢᵢ | E₄* | E_{f} |
| 4294 | E_{1b}E₂EᵢE₄ | Eᵥᵢᵢ | E₄* | E_{f} |
| 4295 | E₁ₐE₂EᵢᵢE₄ | Eᵥᵢᵢ | E₄* | E_{f} |
| 4296 | E_{1b}E₂EᵢᵢE₄ | Eᵥᵢᵢ | E₄* | E_{f} |
| 4297 | E₁ₐE₂EᵢᵢE_{iib}E₄ | Eᵥᵢᵢ | E₄* | E_{f} |
| 4298 | E_{1b}E₂EᵢᵢE_{iib}E₄ | Eᵥᵢᵢ | E₄* | E_{f} |
| 4299 | E₁ₐE₂E₃EᵢE₄ | Eᵥᵢᵢ | E₄* | E_{f} |
| 4300 | E_{1b}E₂E₃EᵢE₄ | Eᵥᵢᵢ | E₄* | E_{f} |
| 4301 | E₁ₐE₂E₃EᵢᵢE₄ | Eᵥᵢᵢ | E₄* | E_{f} |
| 4302 | E_{1b}E₂E₃EᵢᵢE₄ | Eᵥᵢᵢ | E₄* | E_{f} |
| 4303 | E₁ₐE₂E₃EᵢᵢE_{iib}E₄ | Eᵥᵢᵢ | E₄* | E_{f} |
| 4304 | E_{1b}E₂E₃EᵢᵢE_{iib}E₄ | Eᵥᵢᵢ | E₄* | E_{f} |
| 4305 | E₁ₐE₂Eᵢᵢᵢ | Eᵥᵢᵢ | E₄* | E_{f} |
| 4306 | E_{1b}E₂Eᵢᵢᵢ | Eᵥᵢᵢ | E₄* | E_{f} |
| 4307 | E₁ₐE₂E₃Eᵢᵢᵢ | Eᵥᵢᵢ | E₄* | E_{f} |
| 4308 | E_{1b}E₂E₃Eᵢᵢᵢ | Eᵥᵢᵢ | E₄* | E_{f} |
| 4309 | E₁ₐE₂EᵢᵢᵢE₄ | Eᵥᵢᵢ | E₄* | E_{f} |
| 4310 | E_{1b}E₂EᵢᵢᵢE₄ | Eᵥᵢᵢ | E₄* | E_{f} |
| 4311 | E₁ₐE₂E₃EᵢᵢᵢE₄ | Eᵥᵢᵢ | E₄* | E_{f} |
| 4312 | E_{1b}E₂E₃EᵢᵢᵢE₄ | Eᵥᵢᵢ | E₄* | E_{f} |
| 4313 | E₁ₐE₂Eᵢᵥ | Eᵥᵢᵢ | E₄* | E_{f} |
| 4314 | E_{1b}E₂Eᵢᵥ | Eᵥᵢᵢ | E₄* | E_{f} |
| 4315 | E₁ₐE₂E₃Eᵢᵥ | Eᵥᵢᵢ | E₄* | E_{f} |
| 4316 | E_{1b}E₂E₃Eᵢᵥ | Eᵥᵢᵢ | E₄* | E_{f} |
| 4317 | E₁ₐE₂EᵢᵥE₄ | Eᵥᵢᵢ | E₄* | E_{f} |
| 4318 | E_{1b}E₂EᵢᵥE₄ | Eᵥᵢᵢ | E₄* | E_{f} |
| 4319 | E₁ₐE₂E₃EᵢᵥE₄ | Eᵥᵢᵢ | E₄* | E_{f} |
| 4320 | E_{1b}E₂E₃EᵢᵥE₄ | Eᵥᵢᵢ | E₄* | E_{f} |
| 4321 | E₁ₐE₂ | Eᵥᵢᵢ | E₅ | E_{f} |
| 4322 | E_{1b}E₂ | Eᵥᵢᵢ | E₅ | E_{f} |
| 4323 | E₁ₐE₂E₃ | Eᵥᵢᵢ | E₅ | E_{f} |
| 4324 | E_{1b}E₂E₃ | Eᵥᵢᵢ | E₅ | E_{f} |
| 4325 | E₁ₐE₂Eᵢ | Eᵥᵢᵢ | E₅ | E_{f} |
| 4326 | E_{1b}E₂Eᵢ | Eᵥᵢᵢ | E₅ | E_{f} |
| 4327 | E₁ₐE₂Eᵢᵢ | Eᵥᵢᵢ | E₅ | E_{f} |
| 4328 | E_{1b}E₂Eᵢᵢ | Eᵥᵢᵢ | E₅ | E_{f} |
| 4329 | E₁ₐE₂EᵢᵢE_{iib} | Eᵥᵢᵢ | E₅ | E_{f} |
| 4330 | E_{1b}E₂EᵢᵢE_{iib} | Eᵥᵢᵢ | E₅ | E_{f} |
| 4331 | E₁ₐE₂E₃Eᵢ | Eᵥᵢᵢ | E₅ | E_{f} |
| 4332 | E_{1b}E₂E₃Eᵢ | Eᵥᵢᵢ | E₅ | E_{f} |
| 4333 | E₁ₐE₂E₃Eᵢᵢ | Eᵥᵢᵢ | E₅ | E_{f} |
| 4334 | E_{1b}E₂E₃Eᵢᵢ | Eᵥᵢᵢ | E₅ | E_{f} |
| 4335 | E₁ₐE₂E₃EᵢᵢE_{iib} | Eᵥᵢᵢ | E₅ | E_{f} |
| 4336 | E_{1b}E₂E₃EᵢᵢE_{iib} | Eᵥᵢᵢ | E₅ | E_{f} |
| 4337 | E₁ₐE₂E₄ | Eᵥᵢᵢ | E₅ | E_{f} |
| 4338 | E_{1b}E₂E₄ | Eᵥᵢᵢ | E₅ | E_{f} |
| 4339 | E₁ₐE₂E₃E₄ | Eᵥᵢᵢ | E₅ | E_{f} |
| 4340 | E_{1b}E₂E₃E₄ | Eᵥᵢᵢ | E₅ | E_{f} |
| 4341 | E₁ₐE₂EᵢE₄ | Eᵥᵢᵢ | E₅ | E_{f} |
| 4342 | E_{1b}E₂EᵢE₄ | Eᵥᵢᵢ | E₅ | E_{f} |
| 4343 | E₁ₐE₂EᵢᵢE₄ | Eᵥᵢᵢ | E₅ | E_{f} |
| 4344 | E_{1b}E₂EᵢᵢE₄ | Eᵥᵢᵢ | E₅ | E_{f} |
| 4345 | E₁ₐE₂EᵢᵢE_{iib}E₄ | Eᵥᵢᵢ | E₅ | E_{f} |
| 4346 | E_{1b}E₂EᵢᵢE_{iib}E₄ | Eᵥᵢᵢ | E₅ | E_{f} |
| 4347 | E₁ₐE₂E₃EᵢE₄ | Eᵥᵢᵢ | E₅ | E_{f} |
| 4348 | E_{1b}E₂E₃EᵢE₄ | Eᵥᵢᵢ | E₅ | E_{f} |
| 4349 | E₁ₐE₂E₃EᵢᵢE₄ | Eᵥᵢᵢ | E₅ | E_{f} |
| 4350 | E_{1b}E₂E₃EᵢᵢE₄ | Eᵥᵢᵢ | E₅ | E_{f} |
| 4351 | E₁ₐE₂E₃EᵢᵢE_{iib}E₄ | Eᵥᵢᵢ | E₅ | E_{f} |
| 4352 | E_{1b}E₂E₃EᵢᵢE_{iib}E₄ | Eᵥᵢᵢ | E₅ | E_{f} |
| 4353 | E₁ₐE₂Eᵢᵢᵢ | Eᵥᵢᵢ | E₅ | E_{f} |
| 4354 | E_{1b}E₂Eᵢᵢᵢ | Eᵥᵢᵢ | E₅ | E_{f} |
| 4355 | E₁ₐE₂E₃Eᵢᵢᵢ | Eᵥᵢᵢ | E₅ | E_{f} |
| 4356 | E_{1b}E₂E₃Eᵢᵢᵢ | Eᵥᵢᵢ | E₅ | E_{f} |
| 4357 | E₁ₐE₂EᵢᵢᵢE₄ | Eᵥᵢᵢ | E₅ | E_{f} |
| 4358 | E_{1b}E₂EᵢᵢᵢE₄ | Eᵥᵢᵢ | E₅ | E_{f} |
| 4359 | E₁ₐE₂E₃EᵢᵢᵢE₄ | Eᵥᵢᵢ | E₅ | E_{f} |
| 4360 | E_{1b}E₂E₃EᵢᵢᵢE₄ | Eᵥᵢᵢ | E₅ | E_{f} |
| 4361 | E₁ₐE₂Eᵢᵥ | Eᵥᵢᵢ | E₅ | E_{f} |
| 4362 | E_{1b}E₂Eᵢᵥ | Eᵥᵢᵢ | E₅ | E_{f} |
| 4363 | E₁ₐE₂E₃Eᵢᵥ | Eᵥᵢᵢ | E₅ | E_{f} |
| 4364 | E_{1b}E₂E₃Eᵢᵥ | Eᵥᵢᵢ | E₅ | E_{f} |
| 4365 | E₁ₐE₂EᵢᵥE₄ | Eᵥᵢᵢ | E₅ | E_{f} |
| 4366 | E_{1b}E₂EᵢᵥE₄ | Eᵥᵢᵢ | E₅ | E_{f} |
| 4367 | E₁ₐE₂E₃EᵢᵥE₄ | Eᵥᵢᵢ | E₅ | E_{f} |
| 4368 | E_{1b}E₂E₃EᵢᵥE₄ | Eᵥᵢᵢ | E₅ | E_{f} |
| 4369 | E₁ₐE₂ | Eᵥᵢᵢ | E₄*E₅ | E_{f} |
| 4370 | E_{1b}E₂ | Eᵥᵢᵢ | E₄*E₅ | E_{f} |
| 4371 | E₁ₐE₂E₃ | Eᵥᵢᵢ | E₄*E₅ | E_{f} |
| 4372 | E_{1b}E₂E₃ | Eᵥᵢᵢ | E₄*E₅ | E_{f} |
| 4373 | E₁ₐE₂Eᵢ | Eᵥᵢᵢ | E₄*E₅ | E_{f} |
| 4374 | E_{1b}E₂Eᵢ | Eᵥᵢᵢ | E₄*E₅ | E_{f} |
| 4375 | E₁ₐE₂Eᵢᵢ | Eᵥᵢᵢ | E₄*E₅ | E_{f} |
| 4376 | E_{1b}E₂Eᵢᵢ | Eᵥᵢᵢ | E₄*E₅ | E_{f} |
| 4377 | E₁ₐE₂EᵢᵢE_{iib} | Eᵥᵢᵢ | E₄*E₅ | E_{f} |
| 4378 | E_{1b}E₂EᵢᵢE_{iib} | Eᵥᵢᵢ | E₄*E₅ | E_{f} |
| 4379 | E₁ₐE₂E₃Eᵢ | Eᵥᵢᵢ | E₄*E₅ | E_{f} |
| 4380 | E_{1b}E₂E₃Eᵢ | Eᵥᵢᵢ | E₄*E₅ | E_{f} |
| 4381 | E₁ₐE₂E₃Eᵢᵢ | Eᵥᵢᵢ | E₄*E₅ | E_{f} |
| 4382 | E_{1b}E₂E₃Eᵢᵢ | Eᵥᵢᵢ | E₄*E₅ | E_{f} |
| 4383 | E₁ₐE₂E₃EᵢᵢE_{iib} | Eᵥᵢᵢ | E₄*E₅ | E_{f} |
| 4384 | E_{1b}E₂E₃EᵢᵢE_{iib} | Eᵥᵢᵢ | E₄*E₅ | E_{f} |
| 4385 | E₁ₐE₂E₄ | Eᵥᵢᵢ | E₄*E₅ | E_{f} |
| 4386 | E_{1b}E₂E₄ | Eᵥᵢᵢ | E₄*E₅ | E_{f} |
| 4387 | E₁ₐE₂E₃E₄ | Eᵥᵢᵢ | E₄*E₅ | E_{f} |
| 4388 | E_{1b}E₂E₃E₄ | Eᵥᵢᵢ | E₄*E₅ | E_{f} |
| 4389 | E₁ₐE₂EᵢE₄ | Eᵥᵢᵢ | E₄*E₅ | E_{f} |
| 4390 | E_{1b}E₂EᵢE₄ | Eᵥᵢᵢ | E₄*E₅ | E_{f} |
| 4391 | E₁ₐE₂EᵢᵢE₄ | Eᵥᵢᵢ | E₄*E₅ | E_{f} |
| 4392 | E_{1b}E₂EᵢᵢE₄ | Eᵥᵢᵢ | E₄*E₅ | E_{f} |
| 4393 | E₁ₐE₂EᵢᵢE_{iib}E₄ | Eᵥᵢᵢ | E₄*E₅ | E_{f} |
| 4394 | E_{1b}E₂EᵢᵢE_{iib}E₄ | Eᵥᵢᵢ | E₄*E₅ | E_{f} |
| 4395 | E₁ₐE₂E₃EᵢE₄ | Eᵥᵢᵢ | E₄*E₅ | E_{f} |
| 4396 | E_{1b}E₂E₃EᵢE₄ | Eᵥᵢᵢ | E₄*E₅ | E_{f} |
| 4397 | E₁ₐE₂E₃EᵢᵢE₄ | Eᵥᵢᵢ | E₄*E₅ | E_{f} |
| 4398 | E_{1b}E₂E₃EᵢᵢE₄ | Eᵥᵢᵢ | E₄*E₅ | E_{f} |
| 4399 | E₁ₐE₂E₃EᵢᵢE_{iib}E₄ | Eᵥᵢᵢ | E₄*E₅ | E_{f} |
| 4400 | E_{1b}E₂E₃EᵢᵢE_{iib}E₄ | Eᵥᵢᵢ | E₄*E₅ | E_{f} |
| 4401 | E₁ₐE₂Eᵢᵢᵢ | Eᵥᵢᵢ | E₄*E₅ | E_{f} |
| 4402 | E_{1b}E₂Eᵢᵢᵢ | Eᵥᵢᵢ | E₄*E₅ | E_{f} |
| 4403 | E₁ₐE₂E₃Eᵢᵢᵢ | Eᵥᵢᵢ | E₄*E₅ | E_{f} |
| 4404 | E_{1b}E₂E₃Eᵢᵢᵢ | Eᵥᵢᵢ | E₄*E₅ | E_{f} |
| 4405 | E₁ₐE₂EᵢᵢᵢE₄ | Eᵥᵢᵢ | E₄*E₅ | E_{f} |
| 4406 | E_{1b}E₂EᵢᵢᵢE₄ | Eᵥᵢᵢ | E₄*E₅ | E_{f} |
| 4407 | E₁ₐE₂E₃EᵢᵢᵢE₄ | Eᵥᵢᵢ | E₄*E₅ | E_{f} |
| 4408 | E_{1b}E₂E₃EᵢᵢᵢE₄ | Eᵥᵢᵢ | E₄*E₅ | E_{f} |
| 4409 | E₁ₐE₂Eᵢᵥ | Eᵥᵢᵢ | E₄*E₅ | E_{f} |
| 4410 | E_{1b}E₂Eᵢᵥ | Eᵥᵢᵢ | E₄*E₅ | E_{f} |
| 4411 | E₁ₐE₂E₃Eᵢᵥ | Eᵥᵢᵢ | E₄*E₅ | E_{f} |
| 4412 | E_{1b}E₂E₃Eᵢᵥ | Eᵥᵢᵢ | E₄*E₅ | E_{f} |
| 4413 | E₁ₐE₂EᵢᵥE₄ | Eᵥᵢᵢ | E₄*E₅ | E_{f} |
| 4414 | E_{1b}E₂EᵢᵥE₄ | Eᵥᵢᵢ | E₄*E₅ | E_{f} |
| 4415 | E₁ₐE₂E₃EᵢᵥE₄ | Eᵥᵢᵢ | E₄*E₅ | E_{f} |
| 4416 | E_{1b}E₂E₃EᵢᵥE₄ | Eᵥᵢᵢ | E₄*E₅ | E_{f} |
| 4417 | E₁ₐE₂ | AlkLEᵥᵢᵢ | | E_{f} |
| 4418 | E_{1b}E₂ | AlkLEᵥᵢᵢ | | E_{f} |
| 4419 | E₁ₐE₂E₃ | AlkLEᵥᵢᵢ | | E_{f} |
| 4420 | E_{1b}E₂E₃ | AlkLEᵥᵢᵢ | | E_{f} |
| 4421 | E₁ₐE₂Eᵢ | AlkLEᵥᵢᵢ | | E_{f} |
| 4422 | E_{1b}E₂Eᵢ | AlkLEᵥᵢᵢ | | E_{f} |
| 4423 | E₁ₐE₂Eᵢᵢ | AlkLEᵥᵢᵢ | | E_{f} |
| 4424 | E_{1b}E₂Eᵢᵢ | AlkLEᵥᵢᵢ | | E_{f} |
| 4425 | E₁ₐE₂EᵢᵢE_{iib} | AlkLEᵥᵢᵢ | | E_{f} |
| 4426 | E_{1b}E₂EᵢᵢE_{iib} | AlkLEᵥᵢᵢ | | E_{f} |
| 4427 | E₁ₐE₂E₃Eᵢ | AlkLEᵥᵢᵢ | | E_{f} |
| 4428 | E_{1b}E₂E₃Eᵢ | AlkLEᵥᵢᵢ | | E_{f} |
| 4429 | E₁ₐE₂E₃Eᵢᵢ | AlkLEᵥᵢᵢ | | E_{f} |
| 4430 | E_{1b}E₂E₃Eᵢᵢ | AlkLEᵥᵢᵢ | | E_{f} |
| 4431 | E₁ₐE₂E₃EᵢᵢE_{iib} | AlkLEᵥᵢᵢ | | E_{f} |
| 4432 | E_{1b}E₂E₃EᵢᵢE_{iib} | AlkLEᵥᵢᵢ | | E_{f} |
| 4433 | E₁ₐE₂E₄ | AlkLEᵥᵢᵢ | | E_{f} |
| 4434 | E_{1b}E₂E₄ | AlkLEᵥᵢᵢ | | E_{f} |
| 4435 | E₁ₐE₂E₃E₄ | AlkLEᵥᵢᵢ | | E_{f} |
| 4436 | E_{1b}E₂E₃E₄ | AlkLEᵥᵢᵢ | | E_{f} |
| 4437 | E₁ₐE₂EᵢE₄ | AlkLEᵥᵢᵢ | | E_{f} |
| 4438 | E_{1b}E₂EᵢE₄ | AlkLEᵥᵢᵢ | | E_{f} |
| 4439 | E₁ₐE₂EᵢᵢE₄ | AlkLEᵥᵢᵢ | | E_{f} |
| 4440 | E_{1b}E₂EᵢᵢE₄ | AlkLEᵥᵢᵢ | | E_{f} |
| 4441 | E₁ₐE₂EᵢᵢE_{iib}E₄ | AlkLEᵥᵢᵢ | | E_{f} |
| 4442 | E_{1b}E₂EᵢᵢE_{iib}E₄ | AlkLEᵥᵢᵢ | | E_{f} |
| 4443 | E₁ₐE₂E₃EᵢE₄ | AlkLEᵥᵢᵢ | | E_{f} |
| 4444 | E_{1b}E₂E₃EᵢE₄ | AlkLEᵥᵢᵢ | | E_{f} |
| 4445 | E₁ₐE₂E₃EᵢᵢE₄ | AlkLEᵥᵢᵢ | | E_{f} |
| 4446 | E_{1b}E₂E₃EᵢᵢE₄ | AlkLEᵥᵢᵢ | | E_{f} |
| 4447 | E₁ₐE₂E₃EᵢᵢE_{iib}E₄ | AlkLEᵥᵢᵢ | | E_{f} |
| 4448 | E_{1b}E₂E₃EᵢᵢE_{iib}E₄ | AlkLEᵥᵢᵢ | | E_{f} |
| 4449 | E₁ₐE₂Eᵢᵢᵢ | AlkLEᵥᵢᵢ | | E_{f} |
| 4450 | E_{1b}E₂Eᵢᵢᵢ | AlkLEᵥᵢᵢ | | E_{f} |
| 4451 | E₁ₐE₂E₃Eᵢᵢᵢ | AlkLEᵥᵢᵢ | | E_{f} |
| 4452 | E_{1b}E₂E₃Eᵢᵢᵢ | AlkLEᵥᵢᵢ | | E_{f} |
| 4453 | E₁ₐE₂EᵢᵢᵢE₄ | AlkLEᵥᵢᵢ | | E_{f} |
| 4454 | E_{1b}E₂EᵢᵢᵢE₄ | AlkLEᵥᵢᵢ | | E_{f} |
| 4455 | E₁ₐE₂E₃EᵢᵢᵢE₄ | AlkLEᵥᵢᵢ | | E_{f} |
| 4456 | E_{1b}E₂E₃EᵢᵢᵢE₄ | AlkLEᵥᵢᵢ | | E_{f} |
| 4457 | E₁ₐE₂Eᵢᵥ | AlkLEᵥᵢᵢ | | E_{f} |
| 4458 | E_{1b}E₂Eᵢᵥ | AlkLEᵥᵢᵢ | | E_{f} |
| 4459 | E₁ₐE₂E₃Eᵢᵥ | AlkLEᵥᵢᵢ | | E_{f} |
| 4460 | E_{1b}E₂E₃Eᵢᵥ | AlkLEᵥᵢᵢ | | E_{f} |
| 4461 | E₁ₐE₂EᵢᵥE₄ | AlkLEᵥᵢᵢ | | E_{f} |
| 4462 | E_{1b}E₂EᵢᵥE₄ | AlkLEᵥᵢᵢ | | E_{f} |
| 4463 | E₁ₐE₂E₃EᵢᵥE₄ | AlkLEᵥᵢᵢ | | E_{f} |
| 4464 | E_{1b}E₂E₃EᵢᵥE₄ | AlkLEᵥᵢᵢ | | E_{f} |
| 4465 | E₁ₐE₂ | AlkLEᵥᵢᵢ | E₄* | E_{f} |
| 4466 | E_{1b}E₂ | AlkLEᵥᵢᵢ | E₄* | E_{f} |
| 4467 | E₁ₐE₂E₃ | AlkLEᵥᵢᵢ | E₄* | E_{f} |
| 4468 | E_{1b}E₂E₃ | AlkLEᵥᵢᵢ | E₄* | E_{f} |
| 4469 | E₁ₐE₂Eᵢ | AlkLEᵥᵢᵢ | E₄* | E_{f} |
| 4470 | E_{1b}E₂Eᵢ | AlkLEᵥᵢᵢ | E₄* | E_{f} |
| 4471 | E₁ₐE₂Eᵢᵢ | AlkLEᵥᵢᵢ | E₄* | E_{f} |
| 4472 | E_{1b}E₂Eᵢᵢ | AlkLEᵥᵢᵢ | E₄* | E_{f} |
| 4473 | E₁ₐE₂EᵢᵢE_{iib} | AlkLEᵥᵢᵢ | E₄* | E_{f} |
| 4474 | E_{1b}E₂EᵢᵢE_{iib} | AlkLEᵥᵢᵢ | E₄* | E_{f} |
| 4475 | E₁ₐE₂E₃Eᵢ | AlkLEᵥᵢᵢ | E₄* | E_{f} |
| 4476 | E_{1b}E₂E₃Eᵢ | AlkLEᵥᵢᵢ | E₄* | E_{f} |
| 4477 | E₁ₐE₂E₃Eᵢᵢ | AlkLEᵥᵢᵢ | E₄* | E_{f} |
| 4478 | E_{1b}E₂E₃Eᵢᵢ | AlkLEᵥᵢᵢ | E₄* | E_{f} |
| 4479 | E₁ₐE₂E₃EᵢᵢE_{iib} | AlkLEᵥᵢᵢ | E₄* | E_{f} |
| 4480 | E_{1b}E₂E₃EᵢᵢE_{iib} | AlkLEᵥᵢᵢ | E₄* | E_{f} |
| 4481 | E₁ₐE₂E₄ | AlkLEᵥᵢᵢ | E₄* | E_{f} |
| 4482 | E_{1b}E₂E₄ | AlkLEᵥᵢᵢ | E₄* | E_{f} |
| 4483 | E₁ₐE₂E₃E₄ | AlkLEᵥᵢᵢ | E₄* | E_{f} |
| 4484 | E_{1b}E₂E₃E₄ | AlkLEᵥᵢᵢ | E₄* | E_{f} |
| 4485 | E₁ₐE₂EᵢE₄ | AlkLEᵥᵢᵢ | E₄* | E_{f} |
| 4486 | E_{1b}E₂EᵢE₄ | AlkLEᵥᵢᵢ | E₄* | E_{f} |
| 4487 | E₁ₐE₂EᵢᵢE₄ | AlkLEᵥᵢᵢ | E₄* | E_{f} |
| 4488 | E_{1b}E₂EᵢᵢE₄ | AlkLEᵥᵢᵢ | E₄* | E_{f} |
| 4489 | E₁ₐE₂EᵢᵢE_{iib}E₄ | AlkLEᵥᵢᵢ | E₄* | E_{f} |
| 4490 | E_{1b}E₂EᵢᵢE_{iib}E₄ | AlkLEᵥᵢᵢ | E₄* | E_{f} |
| 4491 | E₁ₐE₂E₃EᵢE₄ | AlkLEᵥᵢᵢ | E₄* | E_{f} |
| 4492 | E_{1b}E₂E₃EᵢE₄ | AlkLEᵥᵢᵢ | E₄* | E_{f} |
| 4493 | E₁ₐE₂E₃EᵢᵢE₄ | AlkLEᵥᵢᵢ | E₄* | E_{f} |
| 4494 | E_{1b}E₂E₃EᵢᵢE₄ | AlkLEᵥᵢᵢ | E₄* | E_{f} |
| 4495 | E₁ₐE₂E₃EᵢᵢE_{iib}E₄ | AlkLEᵥᵢᵢ | E₄* | E_{f} |
| 4496 | E_{1b}E₂E₃EᵢᵢE_{iib}E₄ | AlkLEᵥᵢᵢ | E₄* | E_{f} |
| 4497 | E₁ₐE₂Eᵢᵢᵢ | AlkLEᵥᵢᵢ | E₄* | E_{f} |
| 4498 | E_{1b}E₂Eᵢᵢᵢ | AlkLEᵥᵢᵢ | E₄* | E_{f} |
| 4499 | E₁ₐE₂E₃Eᵢᵢᵢ | AlkLEᵥᵢᵢ | E₄* | E_{f} |
| 4500 | E_{1b}E₂E₃Eᵢᵢᵢ | AlkLEᵥᵢᵢ | E₄* | E_{f} |
| 4501 | E₁ₐE₂EᵢᵢᵢE₄ | AlkLEᵥᵢᵢ | E₄* | E_{f} |
| 4502 | E_{1b}E₂EᵢᵢᵢE₄ | AlkLEᵥᵢᵢ | E₄* | E_{f} |
| 4503 | E₁ₐE₂E₃EᵢᵢᵢE₄ | AlkLEᵥᵢᵢ | E₄* | E_{f} |
| 4504 | E_{1b}E₂E₃EᵢᵢᵢE₄ | AlkLEᵥᵢᵢ | E₄* | E_{f} |
| 4505 | E₁ₐE₂Eᵢᵥ | AlkLEᵥᵢᵢ | E₄* | E_{f} |
| 4506 | E_{1b}E₂Eᵢᵥ | AlkLEᵥᵢᵢ | E₄* | E_{f} |
| 4507 | E₁ₐE₂E₃Eᵢᵥ | AlkLEᵥᵢᵢ | E₄* | E_{f} |
| 4508 | E_{1b}E₂E₃Eᵢᵥ | AlkLEᵥᵢᵢ | E₄* | E_{f} |
| 4509 | E₁ₐE₂EᵢᵥE₄ | AlkLEᵥᵢᵢ | E₄* | E_{f} |
| 4510 | E_{1b}E₂EᵢᵥE₄ | AlkLEᵥᵢᵢ | E₄* | E_{f} |
| 4511 | E₁ₐE₂E₃EᵢᵥE₄ | AlkLEᵥᵢᵢ | E₄* | E_{f} |
| 4512 | E_{1b}E₂E₃EᵢᵥE₄ | AlkLEᵥᵢᵢ | E₄* | E_{f} |
| 4513 | E₁ₐE₂ | AlkLEᵥᵢᵢ | E₅ | E_{f} |
| 4514 | E_{1b}E₂ | AlkLEᵥᵢᵢ | E₅ | E_{f} |
| 4515 | E₁ₐE₂E₃ | AlkLEᵥᵢᵢ | E₅ | E_{f} |
| 4516 | E_{1b}E₂E₃ | AlkLEᵥᵢᵢ | E₅ | E_{f} |
| 4517 | E₁ₐE₂Eᵢ | AlkLEᵥᵢᵢ | E₅ | E_{f} |
| 4518 | E_{1b}E₂Eᵢ | AlkLEᵥᵢᵢ | E₅ | E_{f} |
| 4519 | E₁ₐE₂Eᵢᵢ | AlkLEᵥᵢᵢ | E₅ | E_{f} |
| 4520 | E_{1b}E₂Eᵢᵢ | AlkLEᵥᵢᵢ | E₅ | E_{f} |
| 4521 | E₁ₐE₂EᵢᵢE_{iib} | AlkLEᵥᵢᵢ | E₅ | E_{f} |
| 4522 | E_{1b}E₂EᵢᵢE_{iib} | AlkLEᵥᵢᵢ | E₅ | E_{f} |
| 4523 | E₁ₐE₂E₃Eᵢ | AlkLEᵥᵢᵢ | E₅ | E_{f} |
| 4524 | E_{1b}E₂E₃Eᵢ | AlkLEᵥᵢᵢ | E₅ | E_{f} |
| 4525 | E₁ₐE₂E₃Eᵢᵢ | AlkLEᵥᵢᵢ | E₅ | E_{f} |
| 4526 | E_{1b}E₂E₃Eᵢᵢ | AlkLEᵥᵢᵢ | E₅ | E_{f} |
| 4527 | E₁ₐE₂E₃EᵢᵢE_{iib} | AlkLEᵥᵢᵢ | E₅ | E_{f} |
| 4528 | E_{1b}E₂E₃EᵢᵢE_{iib} | AlkLEᵥᵢᵢ | E₅ | E_{f} |
| 4529 | E₁ₐE₂E₄ | AlkLEᵥᵢᵢ | E₅ | E_{f} |
| 4530 | E_{1b}E₂E₄ | AlkLEᵥᵢᵢ | E₅ | E_{f} |
| 4531 | E₁ₐE₂E₃E₄ | AlkLEᵥᵢᵢ | E₅ | E_{f} |
| 4532 | E_{1b}E₂E₃E₄ | AlkLEᵥᵢᵢ | E₅ | E_{f} |
| 4533 | E₁ₐE₂EᵢE₄ | AlkLEᵥᵢᵢ | E₅ | E_{f} |
| 4534 | E_{1b}E₂EᵢE₄ | AlkLEᵥᵢᵢ | E₅ | E_{f} |
| 4535 | E₁ₐE₂EᵢᵢE₄ | AlkLEᵥᵢᵢ | E₅ | E_{f} |
| 4536 | E_{1b}E₂EᵢᵢE₄ | AlkLEᵥᵢᵢ | E₅ | E_{f} |
| 4537 | E₁ₐE₂EᵢᵢE_{iib}E₄ | AlkLEᵥᵢᵢ | E₅ | E_{f} |
| 4538 | E_{1b}E₂EᵢᵢE_{iib}E₄ | AlkLEᵥᵢᵢ | E₅ | E_{f} |
| 4539 | E₁ₐE₂E₃EᵢE₄ | AlkLEᵥᵢᵢ | E₅ | E_{f} |
| 4540 | E_{1b}E₂E₃EᵢE₄ | AlkLEᵥᵢᵢ | E₅ | E_{f} |
| 4541 | E₁ₐE₂E₃EᵢᵢE₄ | AlkLEᵥᵢᵢ | E₅ | E_{f} |
| 4542 | E_{1b}E₂E₃EᵢᵢE₄ | AlkLEᵥᵢᵢ | E₅ | E_{f} |
| 4543 | E₁ₐE₂E₃EᵢᵢE_{iib}E₄ | AlkLEᵥᵢᵢ | E₅ | E_{f} |
| 4544 | E_{1b}E₂E₃EᵢᵢE_{iib}E₄ | AlkLEᵥᵢᵢ | E₅ | E_{f} |
| 4545 | E₁ₐE₂Eᵢᵢᵢ | AlkLEᵥᵢᵢ | E₅ | E_{f} |
| 4546 | E_{1b}E₂Eᵢᵢᵢ | AlkLEᵥᵢᵢ | E₅ | E_{f} |
| 4547 | E₁ₐE₂E₃Eᵢᵢᵢ | AlkLEᵥᵢᵢ | E₅ | E_{f} |
| 4548 | E_{1b}E₂E₃Eᵢᵢᵢ | AlkLEᵥᵢᵢ | E₅ | E_{f} |
| 4549 | E₁ₐE₂EᵢᵢᵢE₄ | AlkLEᵥᵢᵢ | E₅ | E_{f} |
| 4550 | E_{1b}E₂EᵢᵢᵢE₄ | AlkLEᵥᵢᵢ | E₅ | E_{f} |
| 4551 | E₁ₐE₂E₃EᵢᵢᵢE₄ | AlkLEᵥᵢᵢ | E₅ | E_{f} |
| 4552 | E_{1b}E₂E₃EᵢᵢᵢE₄ | AlkLEᵥᵢᵢ | E₅ | E_{f} |
| 4553 | E₁ₐE₂Eᵢᵥ | AlkLEᵥᵢᵢ | E₅ | E_{f} |
| 4554 | E_{1b}E₂Eᵢᵥ | AlkLEᵥᵢᵢ | E₅ | E_{f} |
| 4555 | E₁ₐE₂E₃Eᵢᵥ | AlkLEᵥᵢᵢ | E₅ | E_{f} |
| 4556 | E_{1b}E₂E₃Eᵢᵥ | AlkLEᵥᵢᵢ | E₅ | E_{f} |
| 4557 | E₁ₐE₂EᵢᵥE₄ | AlkLEᵥᵢᵢ | E₅ | E_{f} |
| 4558 | E_{1b}E₂EᵢᵥE₄ | AlkLEᵥᵢᵢ | E₅ | E_{f} |
| 4559 | E₁ₐE₂E₃EᵢᵥE₄ | AlkLEᵥᵢᵢ | E₅ | E_{f} |
| 4560 | E_{1b}E₂E₃EᵢᵥE₄ | AlkLEᵥᵢᵢ | E₅ | E_{f} |
| 4561 | E₁ₐE₂ | AlkLEᵥᵢᵢ | E₄*E₅ | E_{f} |
| 4562 | E_{1b}E₂ | AlkLEᵥᵢᵢ | E₄*E₅ | E_{f} |
| 4563 | E₁ₐE₂E₃ | AlkLEᵥᵢᵢ | E₄*E₅ | E_{f} |
| 4564 | E_{1b}E₂E₃ | AlkLEᵥᵢᵢ | E₄*E₅ | E_{f} |
| 4565 | E₁ₐE₂Eᵢ | AlkLEᵥᵢᵢ | E₄*E₅ | E_{f} |
| 4566 | E_{1b}E₂Eᵢ | AlkLEᵥᵢᵢ | E₄*E₅ | E_{f} |
| 4567 | E₁ₐE₂Eᵢᵢ | AlkLEᵥᵢᵢ | E₄*E₅ | E_{f} |
| 4568 | E_{1b}E₂Eᵢᵢ | AlkLEᵥᵢᵢ | E₄*E₅ | E_{f} |
| 4569 | E₁ₐE₂EᵢᵢE_{iib} | AlkLEᵥᵢᵢ | E₄*E₅ | E_{f} |
| 4570 | E_{1b}E₂EᵢᵢE_{iib} | AlkLEᵥᵢᵢ | E₄*E₅ | E_{f} |
| 4571 | E₁ₐE₂E₃Eᵢ | AlkLEᵥᵢᵢ | E₄*E₅ | E_{f} |
| 4572 | E_{1b}E₂E₃Eᵢ | AlkLEᵥᵢᵢ | E₄*E₅ | E_{f} |
| 4573 | E₁ₐE₂E₃Eᵢᵢ | AlkLEᵥᵢᵢ | E₄*E₅ | E_{f} |
| 4574 | E_{1b}E₂E₃Eᵢᵢ | AlkLEᵥᵢᵢ | E₄*E₅ | E_{f} |
| 4575 | E₁ₐE₂E₃EᵢᵢE_{iib} | AlkLEᵥᵢᵢ | E₄*E₅ | E_{f} |
| 4576 | E_{1b}E₂E₃EᵢᵢE_{iib} | AlkLEᵥᵢᵢ | E₄*E₅ | E_{f} |
| 4577 | E₁ₐE₂E₄ | AlkLEᵥᵢᵢ | E₄*E₅ | E_{f} |
| 4578 | E_{1b}E₂E₄ | AlkLEᵥᵢᵢ | E₄*E₅ | E_{f} |
| 4579 | E₁ₐE₂E₃E₄ | AlkLEᵥᵢᵢ | E₄*E₅ | E_{f} |
| 4580 | E_{1b}E₂E₃E₄ | AlkLEᵥᵢᵢ | E₄*E₅ | E_{f} |
| 4581 | E₁ₐE₂EᵢE₄ | AlkLEᵥᵢᵢ | E₄*E₅ | E_{f} |
| 4582 | E_{1b}E₂EᵢE₄ | AlkLEᵥᵢᵢ | E₄*E₅ | E_{f} |
| 4583 | E₁ₐE₂EᵢᵢE₄ | AlkLEᵥᵢᵢ | E₄*E₅ | E_{f} |
| 4584 | E_{1b}E₂EᵢᵢE₄ | AlkLEᵥᵢᵢ | E₄*E₅ | E_{f} |
| 4585 | E₁ₐE₂EᵢᵢE_{iib}E₄ | AlkLEᵥᵢᵢ | E₄*E₅ | E_{f} |
| 4586 | E_{1b}E₂EᵢᵢE_{iib}E₄ | AlkLEᵥᵢᵢ | E₄*E₅ | E_{f} |
| 4587 | E₁ₐE₂E₃EᵢE₄ | AlkLEᵥᵢᵢ | E₄*E₅ | E_{f} |
| 4588 | E_{1b}E₂E₃EᵢE₄ | AlkLEᵥᵢᵢ | E₄*E₅ | E_{f} |
| 4589 | E₁ₐE₂E₃EᵢᵢE₄ | AlkLEᵥᵢᵢ | E₄*E₅ | E_{f} |
| 4590 | E_{1b}E₂E₃EᵢᵢE₄ | AlkLEᵥᵢᵢ | E₄*E₅ | E_{f} |
| 4591 | E₁ₐE₂E₃EᵢᵢE_{iib}E₄ | AlkLEᵥᵢᵢ | E₄*E₅ | E_{f} |
| 4592 | E_{1b}E₂E₃EᵢᵢE_{iib}E₄ | AlkLEᵥᵢᵢ | E₄*E₅ | E_{f} |
| 4593 | E₁ₐE₂Eᵢᵢᵢ | AlkLEᵥᵢᵢ | E₄*E₅ | E_{f} |
| 4594 | E_{1b}E₂Eᵢᵢᵢ | AlkLEᵥᵢᵢ | E₄*E₅ | E_{f} |
| 4595 | E₁ₐE₂E₃Eᵢᵢᵢ | AlkLEᵥᵢᵢ | E₄*E₅ | E_{f} |
| 4596 | E_{1b}E₂E₃Eᵢᵢᵢ | AlkLEᵥᵢᵢ | E₄*E₅ | E_{f} |
| 4597 | E₁ₐE₂EᵢᵢᵢE₄ | AlkLEᵥᵢᵢ | E₄*E₅ | E_{f} |
| 4598 | E_{1b}E₂EᵢᵢᵢE₄ | AlkLEᵥᵢᵢ | E₄*E₅ | E_{f} |
| 4599 | E₁ₐE₂E₃EᵢᵢᵢE₄ | AlkLEᵥᵢᵢ | E₄*E₅ | E_{f} |
| 4600 | E_{1b}E₂E₃EᵢᵢᵢE₄ | AlkLEᵥᵢᵢ | E₄*E₅ | E_{f} |
| 4601 | E₁ₐE₂Eᵢᵥ | AlkLEᵥᵢᵢ | E₄*E₅ | E_{f} |
| 4602 | E_{1b}E₂Eᵢᵥ | AlkLEᵥᵢᵢ | E₄*E₅ | E_{f} |
| 4603 | E₁ₐE₂E₃Eᵢᵥ | AlkLEᵥᵢᵢ | E₄*E₅ | E_{f} |
| 4604 | E_{1b}E₂E₃Eᵢᵥ | AlkLEᵥᵢᵢ | E₄*E₅ | E_{f} |
| 4605 | E₁ₐE₂EᵢᵥE₄ | AlkLEᵥᵢᵢ | E₄*E₅ | E_{f} |
| 4606 | E_{1b}E₂EᵢᵥE₄ | AlkLEᵥᵢᵢ | E₄*E₅ | E_{f} |
| 4607 | E₁ₐE₂E₃EᵢᵥE₄ | AlkLEᵥᵢᵢ | E₄*E₅ | E_{f} |
| 4608 | E_{1b}E₂E₃EᵢᵥE₄ | AlkLEᵥᵢᵢ | E₄*E₅ | E_{f} |

Microorganisms quite especially preferred according to the invention (abbreviated as MO) are outstandingly suitable for the production of ω-hydroxycarboxylic acids or ω-oxocarboxylic acids and have increased or decreased enzyme activities (abbreviated as E) described in the following table, where these can in addition advantageously be combined with an increased enzyme activity compared to the wild type of the microorganism, which is described for the 3-ketoacyl-ACP (Acyl Carrrier Protein) synthase III (EC 2.3.1.41), in particular that from plants, preferably that from plants the seeds whereof contain fatty acids with alkyl residues shorter than 14 C atoms, and particularly preferably that from plants of the genera Cuphea, Elaeis, Cocos, Umbellularia and Cinnamomum and gene products selected from AccA, AccB, AccC, AccD, AceE, AceF, Lpd, AcpP, FabA, FabB, FabD, FabF, FabG, FabH, Fabl, FabZ, PanD, PanK, UdhA, PntA or PntB.

Any combinations of at least two of these enzyme activities can advantageously be increased.

In addition, moreover, it can be advantageous if the microorganism is provided with a lower enzyme activity compared to the wild type of the microorganism, which is described for the gene products selected from TdcE, PflA, PflB, PflC, PflD, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlvI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, singly or in any combination.

| | | |
|---|---|---|
| MO | E increased | E decreased |

| | | | | |
|---|---|---|---|---|
| 1 | E₁ₐ | | | |
| 2 | E_{1b} | | | |
| 3 | E₁ₐEᵢ | | | |
| 4 | E_{1b}Eᵢ | | | |
| 5 | E₁ₐEᵢᵢ | | | |
| 6 | E_{1b}Eᵢᵢ | | | |
| 7 | E₁ₐEᵢᵢE_{iib} | | | |
| 8 | E_{1b}EᵢᵢE_{iib} | | | |
| 9 | E₁ₐE₄ | | | |
| 10 | E_{1b}E₄ | | | |
| 11 | E₁ₐEᵢE₄ | | | |
| 12 | E_{1b}EᵢE₄ | | | |
| 13 | E₁ₐEᵢᵢE₄ | | | |
| 14 | E_{1b}EᵢᵢE₄ | | | |
| 15 | E₁ₐEᵢᵢE_{iib}E₄ | | | |
| 16 | E_{1b}EᵢᵢE_{iib}E₄ | | | |
| 17 | E₁ₐEᵢᵢᵢ | | | |
| 18 | E_{1b}Eᵢᵢᵢ | | | |
| 19 | E₁ₐEᵢᵢᵢE₄ | | | |
| 20 | E_{1b}EᵢᵢᵢE₄ | | | |
| 21 | E₁ₐEᵢᵥ | | | |
| 22 | E_{1b}Eᵢᵥ | | | |
| 23 | E₁ₐEᵢᵥE₄ | | | |
| 24 | E_{1b}EᵢᵥE₄ | | | |
| 25 | E₁ₐ | | E₄* | |
| 26 | E_{1b} | | E₄* | |
| 27 | E₁ₐEᵢ | | E₄* | |
| 28 | E_{1b}Eᵢ | | E₄* | |
| 29 | E₁ₐEᵢᵢ | | E₄* | |
| 30 | E_{1b}Eᵢᵢ | | E₄* | |
| 31 | E₁ₐEᵢᵢE_{iib} | | E₄* | |
| 32 | E_{1b}EᵢᵢE_{iib} | | E₄* | |
| 33 | E₁ₐE₄ | | E₄* | |
| 34 | E_{1b}E₄ | | E₄* | |
| 35 | E₁ₐEᵢE₄ | | E₄* | |
| 36 | E_{1b}EᵢE₄ | | E₄* | |
| 37 | E₁ₐEᵢᵢE₄ | | E₄* | |
| 38 | E_{1b}EᵢᵢE₄ | | E₄* | |
| 39 | E₁ₐEᵢᵢE_{iib}E₄ | | E₄* | |
| 40 | E_{1b}EᵢᵢE_{iib}E₄ | | E₄* | |
| 41 | E₁ₐEᵢᵢᵢ | | E₄* | |
| 42 | E_{1b}Eᵢᵢᵢ | | E₄* | |
| 43 | E₁ₐEᵢᵢᵢE₄ | | E₄* | |
| 44 | E_{1b}EᵢᵢᵢE₄ | | E₄* | |
| 45 | E₁ₐEᵢᵥ | | E₄* | |
| 46 | E_{1b}Eᵢᵥ | | E₄* | |
| 47 | E₁ₐEᵢᵥE₄ | | E₄* | |
| 48 | E_{1b}EᵢᵥE₄ | | E₄* | |
| 49 | E₁ₐ | | E₅ | |
| 50 | E_{1b} | | E₅ | |
| 51 | E₁ₐEᵢ | | E₅ | |
| 52 | E_{1b}Eᵢ | | E₅ | |
| 53 | E₁ₐEᵢᵢ | | E₅ | |
| 54 | E_{1b}Eᵢᵢ | | E₅ | |
| 55 | E₁ₐEᵢᵢE_{iib} | | E₅ | |
| 56 | E_{1b}EᵢᵢE_{iib} | | E₅ | |
| 57 | E₁ₐE₄ | | E₅ | |
| 58 | E_{1b}E₄ | | E₅ | |
| 59 | E₁ₐEᵢE₄ | | E₅ | |
| 60 | E_{1b}EᵢE₄ | | E₅ | |
| 61 | E₁ₐEᵢᵢE₄ | | E₅ | |
| 62 | E_{1b}EᵢᵢE₄ | | E₅ | |
| 63 | E₁ₐEᵢᵢE_{iib}E₄ | | E₅ | |
| 64 | E_{1b}EᵢᵢE_{iib}E₄ | | E₅ | |
| 65 | E₁ₐEᵢᵢᵢ | | E₅ | |
| 66 | E_{1b}Eᵢᵢᵢ | | E₅ | |
| 67 | E₁ₐEᵢᵢᵢE₄ | | E₅ | |
| 68 | E_{1b}EᵢᵢᵢE₄ | | E₅ | |
| 69 | E₁ₐEᵢᵥ | | E₅ | |
| 70 | E_{1b}Eᵢᵥ | | E₅ | |
| 71 | E₁ₐEᵢᵥE₄ | | E₅ | |
| 72 | E_{1b}EᵢᵥE₄ | | E₅ | |
| 73 | E₁ₐ | | E₄*E₅ | |
| 74 | E_{1b} | | E₄*E₅ | |
| 75 | E₁ₐEᵢ | | E₄*E₅ | |
| 76 | E_{1b}Eᵢ | | E₄*E₅ | |
| 77 | E₁ₐEᵢᵢ | | E₄*E₅ | |
| 78 | E_{1b}Eᵢᵢ | | E₄*E₅ | |
| 79 | E₁ₐEᵢᵢE_{iib} | | E₄*E₅ | |
| 80 | E_{1b}EᵢᵢE_{iib} | | E₄*E₅ | |
| 81 | E₁ₐE₄ | | E₄*E₅ | |
| 82 | E_{1b}E₄ | | E₄*E₅ | |
| 83 | E₁ₐEᵢE₄ | | E₄*E₅ | |
| 84 | E_{1b}EᵢE₄ | | E₄*E₅ | |
| 85 | E₁ₐEᵢᵢE₄ | | E₄*E₅ | |
| 86 | E_{1b}EᵢᵢE₄ | | E₄*E₅ | |
| 87 | E₁ₐEᵢᵢE_{iib}E₄ | | E₄*E₅ | |
| 88 | E_{1b}EᵢᵢE_{iib}E₄ | | E₄*E₅ | |
| 89 | E₁ₐEᵢᵢᵢ | | E₄*E₅ | |
| 90 | E_{1b}Eᵢᵢᵢ | | E₄*E₅ | |
| 91 | E₁ₐEᵢᵢᵢE₄ | | E₄*E₅ | |
| 92 | E_{1b}EᵢᵢᵢE₄ | | E₄*E₅ | |
| 93 | E₁ₐEᵢᵥ | | E₄*E₅ | |
| 94 | E_{1b}Eᵢᵥ | | E₄*E₅ | |
| 95 | E₁ₐEᵢᵥE₄ | | E₄*E₅ | |
| 96 | E_{1b}EᵢᵥE₄ | | E₄*E₅ | |
| 97 | E₁ₐ | AlkL | | |
| 98 | E_{1b} | AlkL | | |
| 99 | E₁ₐEᵢ | AlkL | | |
| 100 | E_{1b}Eᵢ | AlkL | | |
| 101 | E₁ₐEᵢᵢ | AlkL | | |
| 102 | E_{1b}Eᵢᵢ | AlkL | | |
| 103 | E₁ₐEᵢᵢE_{iib} | AlkL | | |
| 104 | E_{1b}EᵢᵢE_{iib} | AlkL | | |
| 105 | E₁ₐE₄ | AlkL | | |
| 106 | E_{1b}E₄ | AlkL | | |
| 107 | E₁ₐEᵢE₄ | AlkL | | |
| 108 | E_{1b}EᵢE₄ | AlkL | | |
| 109 | E₁ₐEᵢᵢE₄ | AlkL | | |
| 110 | E_{1b}EᵢᵢE₄ | AlkL | | |
| 111 | E₁ₐEᵢᵢE_{iib}E₄ | AlkL | | |
| 112 | E_{1b}EᵢᵢE_{iib}E₄ | AlkL | | |
| 113 | E₁ₐEᵢᵢᵢ | AlkL | | |
| 114 | E_{1b}Eᵢᵢᵢ | AlkL | | |
| 115 | E₁ₐEᵢᵢᵢE₄ | AlkL | | |
| 116 | E_{1b}EᵢᵢᵢE₄ | AlkL | | |
| 117 | E₁ₐEᵢᵥ | AlkL | | |
| 118 | E_{1b}Eᵢᵥ | AlkL | | |
| 119 | E₁ₐEᵢᵥE₄ | AlkL | | |
| 120 | E_{1b}EᵢᵥE₄ | AlkL | | |
| 121 | E₁ₐ | AlkL | E₄* | |
| 122 | E_{1b} | AlkL | E₄* | |
| 123 | E₁ₐEᵢ | AlkL | E₄* | |
| 124 | E_{1b}Eᵢ | AlkL | E₄* | |
| 125 | E₁ₐEᵢᵢ | AlkL | E₄* | |
| 126 | E_{1b}Eᵢᵢ | AlkL | E₄* | |
| 127 | E₁ₐEᵢᵢE_{iib} | AlkL | E₄* | |
| 128 | E_{1b}EᵢᵢE_{iib} | AlkL | E₄* | |
| 129 | E₁ₐE₄ | AlkL | E₄* | |
| 130 | E_{1b}E₄ | AlkL | E₄* | |
| 131 | E₁ₐEᵢE₄ | AlkL | E₄* | |
| 132 | E_{1b}EᵢE₄ | AlkL | E₄* | |
| 133 | E₁ₐEᵢᵢE₄ | AlkL | E₄* | |
| 134 | E_{1b}EᵢᵢE₄ | AlkL | E₄* | |
| 135 | E₁ₐEᵢᵢE_{iib}E₄ | AlkL | E₄* | |
| 136 | E_{1b}EᵢᵢE_{iib}E₄ | AlkL | E₄* | |
| 137 | E₁ₐEᵢᵢᵢ | AlkL | E₄* | |
| 138 | E_{1b}Eᵢᵢᵢ | AlkL | E₄* | |
| 139 | E₁ₐEᵢᵢᵢE₄ | AlkL | E₄* | |
| 140 | E_{1b}EᵢᵢᵢE₄ | AlkL | E₄* | |
| 141 | E₁ₐEᵢᵥ | AlkL | E₄* | |
| 142 | E_{1b}Eᵢᵥ | AlkL | E₄* | |
| 143 | E₁ₐEᵢᵥE₄ | AlkL | E₄* | |
| 144 | E_{1b}EᵢᵥE₄ | AlkL | E₄* | |
| 145 | E₁ₐ | AlkL | E₅ | |
| 146 | E_{1b} | AlkL | E₅ | |
| 147 | E₁ₐEᵢ | AlkL | E₅ | |
| 148 | E_{1b}Eᵢ | AlkL | E₅ | |
| 149 | E₁ₐEᵢᵢ | AlkL | E₅ | |
| 150 | E_{1b}Eᵢᵢ | AlkL | E₅ | |
| 151 | E₁ₐEᵢᵢE_{iib} | AlkL | E₅ | |
| 152 | E_{1b}EᵢᵢE_{iib} | AlkL | E₅ | |
| 153 | E₁ₐE₄ | AlkL | E₅ | |
| 154 | E_{1b}E₄ | AlkL | E₅ | |
| 155 | E₁ₐEᵢE₄ | AlkL | E₅ | |
| 156 | E_{1b}EᵢE₄ | AlkL | E₅ | |
| 157 | E₁ₐEᵢᵢE₄ | AlkL | E₅ | |
| 158 | E_{1b}EᵢᵢE₄ | AlkL | E₅ | |
| 159 | E₁ₐEᵢᵢE_{iib}E₄ | AlkL | E₅ | |
| 160 | E_{1b}EᵢᵢE_{iib}E₄ | AlkL | E₅ | |
| 161 | E₁ₐEᵢᵢᵢ | AlkL | E₅ | |
| 162 | E_{1b}Eᵢᵢᵢ | AlkL | E₅ | |
| 163 | E₁ₐEᵢᵢᵢE₄ | AlkL | E₅ | |
| 164 | E_{1b}EᵢᵢᵢE₄ | AlkL | E₅ | |
| 165 | E₁ₐEᵢᵥ | AlkL | E₅ | |
| 166 | E_{1b}Eᵢᵥ | AlkL | E₅ | |
| 167 | E₁ₐEᵢᵥE₄ | AlkL | E₅ | |
| 168 | E_{1b}EᵢᵥE₄ | AlkL | E₅ | |
| 169 | E₁ₐ | AlkL | E₄*E₅ | |
| 170 | E_{1b} | AlkL | E₄*E₅ | |
| 171 | E₁ₐEᵢ | AlkL | E₄*E₅ | |
| 172 | E_{1b}Eᵢ | AlkL | E₄*E₅ | |
| 173 | E₁ₐEᵢᵢ | AlkL | E₄*E₅ | |
| 174 | E_{1b}Eᵢᵢ | AlkL | E₄*E₅ | |
| 175 | E₁ₐEᵢᵢE_{iib} | AlkL | E₄*E₅ | |
| 176 | E_{1b}EᵢᵢE_{iib} | AlkL | E₄*E₅ | |
| 177 | E₁ₐE₄ | AlkL | E₄*E₅ | |
| 178 | E_{1b}E₄ | AlkL | E₄*E₅ | |
| 179 | E₁ₐEᵢE₄ | AlkL | E₄*E₅ | |
| 180 | E_{1b}EᵢE₄ | AlkL | E₄*E₅ | |
| 181 | E₁ₐEᵢᵢE₄ | AlkL | E₄*E₅ | |
| 182 | E_{1b}EᵢᵢE₄ | AlkL | E₄*E₅ | |
| 183 | E₁ₐEᵢᵢE_{iib}E₄ | AlkL | E₄*E₅ | |
| 184 | E_{1b}EᵢᵢE_{iib}E₄ | AlkL | E₄*E₅ | |
| 185 | E₁ₐEᵢᵢᵢ | AlkL | E₄*E₅ | |
| 186 | E_{1b}Eᵢᵢᵢ | AlkL | E₄*E₅ | |
| 187 | E₁ₐEᵢᵢᵢE₄ | AlkL | E₄*E₅ | |
| 188 | E_{1b}EᵢᵢᵢE₄ | AlkL | E₄*E₅ | |
| 189 | E₁ₐEᵢᵥ | AlkL | E₄*E₅ | |
| 190 | E_{1b}Eᵢᵥ | AlkL | E₄*E₅ | |
| 191 | E₁ₐEᵢᵥE₄ | AlkL | E₄*E₅ | |
| 192 | E_{1b}EᵢᵥE₄ | AlkL | E₄*E₅ | |
| 193 | E₁ₐ | | | Eₐ |
| 194 | E_{1b} | | | Eₐ |
| 195 | E₁ₐEᵢ | | | Eₐ |
| 196 | E_{1b}Eᵢ | | | Eₐ |
| 197 | E₁ₐEᵢᵢ | | | Eₐ |
| 198 | E_{1b}Eᵢᵢ | | | Eₐ |
| 199 | E₁ₐEᵢᵢE_{iib} | | | Eₐ |
| 200 | E_{1b}EᵢᵢE_{iib} | | | Eₐ |
| 201 | E₁ₐE₄ | | | Eₐ |
| 202 | E_{1b}E₄ | | | Eₐ |
| 203 | E₁ₐEᵢE₄ | | | Eₐ |
| 204 | E_{1b}EᵢE₄ | | | Eₐ |
| 205 | E₁ₐEᵢᵢE₄ | | | Eₐ |
| 206 | E_{1b}EᵢᵢE₄ | | | Eₐ |
| 207 | E₁ₐEᵢᵢE_{iib}E₄ | | | Eₐ |
| 208 | E_{1b}EᵢᵢE_{iib}E₄ | | | Eₐ |
| 209 | E₁ₐEᵢᵢᵢ | | | Eₐ |
| 210 | E_{1b}Eᵢᵢᵢ | | | Eₐ |
| 211 | E₁ₐEᵢᵢᵢE₄ | | | Eₐ |
| 212 | E_{1b}EᵢᵢᵢE₄ | | | Eₐ |
| 213 | E₁ₐEᵢᵥ | | | Eₐ |
| 214 | E_{1b}Eᵢᵥ | | | Eₐ |
| 215 | E₁ₐEᵢᵥE₄ | | | Eₐ |
| 216 | E_{1b}EᵢᵥE₄ | | | Eₐ |
| 217 | E₁ₐ | | E₄* | Eₐ |
| 218 | E_{1b} | | E₄* | Eₐ |
| 219 | E₁ₐEᵢ | | E₄* | Eₐ |
| 220 | E_{1b}Eᵢ | | E₄* | Eₐ |
| 221 | E₁ₐEᵢᵢ | | E₄* | Eₐ |
| 222 | E_{1b}Eᵢᵢ | | E₄* | Eₐ |
| 223 | E₁ₐEᵢᵢE_{iib} | | E₄* | Eₐ |
| 224 | E_{1b}EᵢᵢE_{iib} | | E₄* | Eₐ |
| 225 | E₁ₐE₄ | | E₄* | Eₐ |
| 226 | E_{1b}E₄ | | E₄* | Eₐ |
| 227 | E₁ₐEᵢE₄ | | E₄* | Eₐ |
| 228 | E_{1b}EᵢE₄ | | E₄* | Eₐ |
| 229 | E₁ₐEᵢᵢE₄ | | E₄* | Eₐ |
| 230 | E_{1b}EᵢᵢE₄ | | E₄* | Eₐ |
| 231 | E₁ₐEᵢᵢE_{iib}E₄ | | E₄* | Eₐ |
| 232 | E_{1b}EᵢᵢE_{iib}E₄ | | E₄* | Eₐ |
| 233 | E₁ₐEᵢᵢᵢ | | E₄* | Eₐ |
| 234 | E_{1b}Eᵢᵢᵢ | | E₄* | Eₐ |
| 235 | E₁ₐEᵢᵢᵢE₄ | | E₄* | Eₐ |
| 236 | E_{1b}EᵢᵢᵢE₄ | | E₄* | Eₐ |
| 237 | E₁ₐEᵢᵥ | | E₄* | Eₐ |
| 238 | E_{1b}Eᵢᵥ | | E₄* | Eₐ |
| 239 | E₁ₐEᵢᵥE₄ | | E₄* | Eₐ |
| 240 | E_{1b}EᵢᵥE₄ | | E₄* | Eₐ |
| 241 | E₁ₐ | | E₅ | Eₐ |
| 242 | E_{1b} | | E₅ | Eₐ |
| 243 | E₁ₐEᵢ | | E₅ | Eₐ |
| 244 | E_{1b}Eᵢ | | E₅ | Eₐ |
| 245 | E₁ₐEᵢᵢ | | E₅ | Eₐ |
| 246 | E_{1b}Eᵢᵢ | | E₅ | Eₐ |
| 247 | E₁ₐEᵢᵢE_{iib} | | E₅ | Eₐ |
| 248 | E_{1b}EᵢᵢE_{iib} | | E₅ | Eₐ |
| 249 | E₁ₐE₄ | | E₅ | Eₐ |
| 250 | E_{1b}E₄ | | E₅ | Eₐ |
| 251 | E₁ₐEᵢE₄ | | E₅ | Eₐ |
| 252 | E_{1b}EᵢE₄ | | E₅ | Eₐ |
| 253 | E₁ₐEᵢᵢE₄ | | E₅ | Eₐ |
| 254 | E_{1b}EᵢᵢE₄ | | E₅ | Eₐ |
| 255 | E₁ₐEᵢᵢE_{iib}E₄ | | E₅ | Eₐ |
| 256 | E_{1b}EᵢᵢE_{iib}E₄ | | E₅ | Eₐ |
| 257 | E₁ₐEᵢᵢᵢ | | E₅ | Eₐ |
| 258 | E_{1b}Eᵢᵢᵢ | | E₅ | Eₐ |
| 259 | E₁ₐEᵢᵢᵢE₄ | | E₅ | Eₐ |
| 260 | E_{1b}EᵢᵢᵢE₄ | | E₅ | Eₐ |
| 261 | E₁ₐEᵢᵥ | | E₅ | Eₐ |
| 262 | E_{1b}Eᵢᵥ | | E₅ | Eₐ |
| 263 | E₁ₐEᵢᵥE₄ | | E₅ | Eₐ |
| 264 | E_{1b}EᵢᵥE₄ | | E₅ | Eₐ |
| 265 | E₁ₐ | | E₄*E₅ | Eₐ |
| 266 | E_{1b} | | E₄*E₅ | Eₐ |
| 267 | E₁ₐEᵢ | | E₄*E₅ | Eₐ |
| 268 | E_{1b}Eᵢ | | E₄*E₅ | Eₐ |
| 269 | E₁ₐEᵢᵢ | | E₄*E₅ | Eₐ |
| 270 | E_{1b}Eᵢᵢ | | E₄*E₅ | Eₐ |
| 271 | E₁ₐEᵢᵢE_{iib} | | E₄*E₅ | Eₐ |
| 272 | E_{1b}EᵢᵢE_{iib} | | E₄*E₅ | Eₐ |
| 273 | E₁ₐE₄ | | E₄*E₅ | Eₐ |
| 274 | E_{1b}E₄ | | E₄*E₅ | Eₐ |
| 275 | E₁ₐEᵢE₄ | | E₄*E₅ | Eₐ |
| 276 | E_{1b}EᵢE₄ | | E₄*E₅ | Eₐ |
| 277 | E₁ₐEᵢᵢE₄ | | E₄*E₅ | Eₐ |
| 278 | E_{1b}EᵢᵢE₄ | | E₄*E₅ | Eₐ |
| 279 | E₁ₐEᵢᵢE_{iib}E₄ | | E₄*E₅ | Eₐ |
| 280 | E_{1b}EᵢᵢE_{iib}E₄ | | E₄*E₅ | Eₐ |
| 281 | E₁ₐEᵢᵢᵢ | | E₄*E₅ | Eₐ |
| 282 | E_{1b}Eᵢᵢᵢ | | E₄*E₅ | Eₐ |
| 283 | E₁ₐEᵢᵢᵢE₄ | | E₄*E₅ | Eₐ |
| 284 | E_{1b}EᵢᵢᵢE₄ | | E₄*E₅ | Eₐ |
| 285 | E₁ₐEᵢᵥ | | E₄*E₅ | Eₐ |
| 286 | E_{1b}Eᵢᵥ | | E₄*E₅ | Eₐ |
| 287 | E₁ₐEᵢᵥE₄ | | E₄*E₅ | Eₐ |
| 288 | E_{1b}EᵢᵥE₄ | | E₄*E₅ | Eₐ |
| 289 | E₁ₐ | AlkL | | Eₐ |
| 290 | E_{1b} | AlkL | | Eₐ |
| 291 | E₁ₐEᵢ | AlkL | | Eₐ |
| 292 | E_{1b}Eᵢ | AlkL | | Eₐ |
| 293 | E₁ₐEᵢᵢ | AlkL | | Eₐ |
| 294 | E_{1b}Eᵢᵢ | AlkL | | Eₐ |
| 295 | E₁ₐEᵢᵢE_{iib} | AlkL | | Eₐ |
| 296 | E_{1b}EᵢᵢE_{iib} | AlkL | | Eₐ |
| 297 | E₁ₐE₄ | AlkL | | Eₐ |
| 298 | E_{1b}E₄ | AlkL | | Eₐ |
| 299 | E₁ₐEᵢE₄ | AlkL | | Eₐ |
| 300 | E_{1b}EᵢE₄ | AlkL | | Eₐ |
| 301 | E₁ₐEᵢᵢE₄ | AlkL | | Eₐ |
| 302 | E_{1b}EᵢᵢE₄ | AlkL | | Eₐ |
| 303 | E₁ₐEᵢᵢE_{iib}E₄ | AlkL | | Eₐ |
| 304 | E_{1b}EᵢᵢE_{iib}E₄ | AlkL | | Eₐ |
| 305 | E₁ₐEᵢᵢᵢ | AlkL | | Eₐ |
| 306 | E_{1b}Eᵢᵢᵢ | AlkL | | Eₐ |
| 307 | E₁ₐEᵢᵢᵢE₄ | AlkL | | Eₐ |
| 308 | E_{1b}EᵢᵢᵢE₄ | AlkL | | Eₐ |
| 309 | E₁ₐEᵢᵥ | AlkL | | Eₐ |
| 310 | E_{1b}Eᵢᵥ | AlkL | | Eₐ |
| 311 | E₁ₐEᵢᵥE₄ | AlkL | | Eₐ |
| 312 | E_{1b}EᵢᵥE₄ | AlkL | | Eₐ |
| 313 | E₁ₐ | AlkL | E₄* | Eₐ |
| 314 | E_{1b} | AlkL | E₄* | Eₐ |
| 315 | E₁ₐEᵢ | AlkL | E₄* | Eₐ |
| 316 | E_{1b}Eᵢ | AlkL | E₄* | Eₐ |
| 317 | E₁ₐEᵢᵢ | AlkL | E₄* | Eₐ |
| 318 | E_{1b}Eᵢᵢ | AlkL | E₄* | Eₐ |
| 319 | E₁ₐEᵢᵢE_{iib} | AlkL | E₄* | Eₐ |
| 320 | E_{1b}EᵢᵢE_{iib} | AlkL | E₄* | Eₐ |
| 321 | E₁ₐE₄ | AlkL | E₄* | Eₐ |
| 322 | E_{1b}E₄ | AlkL | E₄* | Eₐ |
| 323 | E₁ₐEᵢE₄ | AlkL | E₄* | Eₐ |
| 324 | E_{1b}EᵢE₄ | AlkL | E₄* | Eₐ |
| 325 | E₁ₐEᵢᵢE₄ | AlkL | E₄* | Eₐ |
| 326 | E_{1b}EᵢᵢE₄ | AlkL | E₄* | Eₐ |
| 327 | E₁ₐEᵢᵢE_{iib}E₄ | AlkL | E₄* | Eₐ |
| 328 | E_{1b}EᵢᵢE_{iib}E₄ | AlkL | E₄* | Eₐ |
| 329 | E₁ₐEᵢᵢᵢ | AlkL | E₄* | Eₐ |
| 330 | E_{1b}Eᵢᵢᵢ | AlkL | E₄* | Eₐ |
| 331 | E₁ₐEᵢᵢᵢE₄ | AlkL | E₄* | Eₐ |
| 332 | E_{1b}EᵢᵢᵢE₄ | AlkL | E₄* | Eₐ |
| 333 | E₁ₐEᵢᵥ | AlkL | E₄* | Eₐ |
| 334 | E_{1b}Eᵢᵥ | AlkL | E₄* | Eₐ |
| 335 | E₁ₐEᵢᵥE₄ | AlkL | E₄* | Eₐ |
| 336 | E_{1b}EᵢᵥE₄ | AlkL | E₄* | Eₐ |
| 337 | E₁ₐ | AlkL | E₅ | Eₐ |
| 338 | E_{1b} | AlkL | E₅ | Eₐ |
| 339 | E₁ₐEᵢ | AlkL | E₅ | Eₐ |
| 340 | E_{1b}Eᵢ | AlkL | E₅ | Eₐ |
| 341 | E₁ₐEᵢᵢ | AlkL | E₅ | Eₐ |
| 342 | E_{1b}Eᵢᵢ | AlkL | E₅ | Eₐ |
| 343 | E₁ₐEᵢᵢE_{iib} | AlkL | E₅ | Eₐ |
| 344 | E_{1b}EᵢᵢE_{iib} | AlkL | E₅ | Eₐ |
| 345 | E₁ₐE₄ | AlkL | E₅ | Eₐ |
| 346 | E_{1b}E₄ | AlkL | E₅ | Eₐ |
| 347 | E₁ₐEᵢE₄ | AlkL | E₅ | Eₐ |
| 348 | E_{1b}EᵢE₄ | AlkL | E₅ | Eₐ |
| 349 | E₁ₐEᵢᵢE₄ | AlkL | E₅ | Eₐ |
| 350 | E_{1b}EᵢᵢE₄ | AlkL | E₅ | Eₐ |
| 351 | E₁ₐEᵢᵢE_{iib}E₄ | AlkL | E₅ | Eₐ |
| 352 | E_{1b}EᵢᵢE_{iib}E₄ | AlkL | E₅ | Eₐ |
| 353 | E₁ₐEᵢᵢᵢ | AlkL | E₅ | Eₐ |
| 354 | E_{1b}Eᵢᵢᵢ | AlkL | E₅ | Eₐ |
| 355 | E₁ₐEᵢᵢᵢE₄ | AlkL | E₅ | Eₐ |
| 356 | E_{1b}EᵢᵢᵢE₄ | AlkL | E₅ | Eₐ |
| 357 | E₁ₐEᵢᵥ | AlkL | E₅ | Eₐ |
| 358 | E_{1b}Eᵢᵥ | AlkL | E₅ | Eₐ |
| 359 | E₁ₐEᵢᵥE₄ | AlkL | E₅ | Eₐ |
| 360 | E_{1b}EᵢᵥE₄ | AlkL | E₅ | Eₐ |
| 361 | E₁ₐ | AlkL | E₄*E₅ | Eₐ |
| 362 | E_{1b} | AlkL | E₄*E₅ | Eₐ |
| 363 | E₁ₐEᵢ | AlkL | E₄*E₅ | Eₐ |
| 364 | E_{1b}Eᵢ | AlkL | E₄*E₅ | Eₐ |
| 365 | E₁ₐEᵢᵢ | AlkL | E₄*E₅ | Eₐ |
| 366 | E_{1b}Eᵢᵢ | AlkL | E₄*E₅ | Eₐ |
| 367 | E₁ₐEᵢᵢE_{iib} | AlkL | E₄*E₅ | Eₐ |
| 368 | E_{1b}EᵢᵢE_{iib} | AlkL | E₄*E₅ | Eₐ |
| 369 | E₁ₐE₄ | AlkL | E₄*E₅ | Eₐ |
| 370 | E_{1b}E₄ | AlkL | E₄*E₅ | Eₐ |
| 371 | E₁ₐEᵢE₄ | AlkL | E₄*E₅ | Eₐ |
| 372 | E_{1b}EᵢE₄ | AlkL | E₄*E₅ | Eₐ |
| 373 | E₁ₐEᵢᵢE₄ | AlkL | E₄*E₅ | Eₐ |
| 374 | E_{1b}EᵢᵢE₄ | AlkL | E₄*E₅ | Eₐ |
| 375 | E₁ₐEᵢᵢE_{iib}E₄ | AlkL | E₄*E₅ | Eₐ |
| 376 | E_{1b}EᵢᵢE_{iib}E₄ | AlkL | E₄*E₅ | Eₐ |
| 377 | E₁ₐEᵢᵢᵢ | AlkL | E₄*E₅ | Eₐ |
| 378 | E_{1b}Eᵢᵢᵢ | AlkL | E₄*E₅ | Eₐ |
| 379 | E₁ₐEᵢᵢᵢE₄ | AlkL | E₄*E₅ | Eₐ |
| 380 | E_{1b}EᵢᵢᵢE₄ | AlkL | E₄*E₅ | Eₐ |
| 381 | E₁ₐEᵢᵥ | AlkL | E₄*E₅ | Eₐ |
| 382 | E_{1b}Eᵢᵥ | AlkL | E₄*E₅ | Eₐ |
| 383 | E₁ₐEᵢᵥE₄ | AlkL | E₄*E₅ | Eₐ |
| 384 | E_{1b}EᵢᵥE₄ | AlkL | E₄*E₅ | Eₐ |
| 385 | E₁ₐ | | | E_{b} |
| 386 | E_{1b} | | | E_{b} |
| 387 | E₁ₐEᵢ | | | E_{b} |
| 388 | E_{1b}Eᵢ | | | E_{b} |
| 389 | E₁ₐEᵢᵢ | | | E_{b} |
| 390 | E_{1b}Eᵢᵢ | | | E_{b} |
| 391 | E₁ₐEᵢᵢE_{iib} | | | E_{b} |
| 392 | E_{1b}EᵢᵢE_{iib} | | | E_{b} |
| 393 | E₁ₐE₄ | | | E_{b} |
| 394 | E_{1b}E₄ | | | E_{b} |
| 395 | E₁ₐEᵢE₄ | | | E_{b} |
| 396 | E_{1b}EᵢE₄ | | | E_{b} |
| 397 | E₁ₐEᵢᵢE₄ | | | E_{b} |
| 398 | E_{1b}EᵢᵢE₄ | | | E_{b} |
| 399 | E₁ₐEᵢᵢE_{iib}E₄ | | | E_{b} |
| 400 | E_{1b}EᵢᵢE_{iib}E₄ | | | E_{b} |
| 401 | E₁ₐEᵢᵢᵢ | | | E_{b} |
| 402 | E_{1b}Eᵢᵢᵢ | | | E_{b} |
| 403 | E₁ₐEᵢᵢᵢE₄ | | | E_{b} |
| 404 | E_{1b}EᵢᵢᵢE₄ | | | E_{b} |
| 405 | E₁ₐEᵢᵥ | | | E_{b} |
| 406 | E_{1b}Eᵢᵥ | | | E_{b} |
| 407 | E₁ₐEᵢᵥE₄ | | | E_{b} |
| 408 | E_{1b}EᵢᵥE₄ | | | E_{b} |
| 409 | E₁ₐ | | E₄* | E_{b} |
| 410 | E_{1b} | | E₄* | E_{b} |
| 411 | E₁ₐEᵢ | | E₄* | E_{b} |
| 412 | E_{1b}Eᵢ | | E₄* | E_{b} |
| 413 | E₁ₐEᵢᵢ | | E₄* | E_{b} |
| 414 | E_{1b}Eᵢᵢ | | E₄* | E_{b} |
| 415 | E₁ₐEᵢᵢE_{iib} | | E₄* | E_{b} |
| 416 | E_{1b}EᵢᵢE_{iib} | | E₄* | E_{b} |
| 417 | E₁ₐE₄ | | E₄* | E_{b} |
| 418 | E_{1b}E₄ | | E₄* | E_{b} |
| 419 | E₁ₐEᵢE₄ | | E₄* | E_{b} |
| 420 | E_{1b}EᵢE₄ | | E₄* | E_{b} |
| 421 | E₁ₐEᵢᵢE₄ | | E₄* | E_{b} |
| 422 | E_{1b}EᵢᵢE₄ | | E₄* | E_{b} |
| 423 | E₁ₐEᵢᵢE_{iib}E₄ | | E₄* | E_{b} |
| 424 | E_{1b}EᵢᵢE_{iib}E₄ | | E₄* | E_{b} |
| 425 | E₁ₐEᵢᵢᵢ | | E₄* | E_{b} |
| 426 | E_{1b}Eᵢᵢᵢ | | E₄* | E_{b} |
| 427 | E₁ₐEᵢᵢᵢE₄ | | E₄* | E_{b} |
| 428 | E_{1b}EᵢᵢᵢE₄ | | E₄* | E_{b} |
| 429 | E₁ₐEᵢᵥ | | E₄* | E_{b} |
| 430 | E_{1b}Eᵢᵥ | | E₄* | E_{b} |
| 431 | E₁ₐEᵢᵥE₄ | | E₄* | E_{b} |
| 432 | E_{1b}EᵢᵥE₄ | | E₄* | E_{b} |
| 433 | E₁ₐ | | E₅ | E_{b} |
| 434 | E_{1b} | | E₅ | E_{b} |
| 435 | E₁ₐEᵢ | | E₅ | E_{b} |
| 436 | E_{1b}Eᵢ | | E₅ | E_{b} |
| 437 | E₁ₐEᵢᵢ | | E₅ | E_{b} |
| 438 | E_{1b}Eᵢᵢ | | E₅ | E_{b} |
| 439 | E₁ₐEᵢᵢE_{iib} | | E₅ | E_{b} |
| 440 | E_{1b}EᵢᵢE_{iib} | | E₅ | E_{b} |
| 441 | E₁ₐE₄ | | E₅ | E_{b} |
| 442 | E_{1b}E₄ | | E₅ | E_{b} |
| 443 | E₁ₐEᵢE₄ | | E₅ | E_{b} |
| 444 | E_{1b}EᵢE₄ | | E₅ | E_{b} |
| 445 | E₁ₐEᵢᵢE₄ | | E₅ | E_{b} |
| 446 | E_{1b}EᵢᵢE₄ | | E₅ | E_{b} |
| 447 | E₁ₐEᵢᵢE_{iib}E₄ | | E₅ | E_{b} |
| 448 | E_{1b}EᵢᵢE_{iib}E₄ | | E₅ | E_{b} |
| 449 | E₁ₐEᵢᵢᵢ | | E₅ | E_{b} |
| 450 | E_{1b}Eᵢᵢᵢ | | E₅ | E_{b} |
| 451 | E₁ₐEᵢᵢᵢE₄ | | E₅ | E_{b} |
| 452 | E_{1b}EᵢᵢᵢE₄ | | E₅ | E_{b} |
| 453 | E₁ₐEᵢᵥ | | E₅ | E_{b} |
| 454 | E_{1b}Eᵢᵥ | | E₅ | E_{b} |
| 455 | E₁ₐEᵢᵥE₄ | | E₅ | E_{b} |
| 456 | E_{1b}EᵢᵥE₄ | | E₅ | E_{b} |
| 457 | E₁ₐ | | E₄*E₅ | E_{b} |
| 458 | E_{1b} | | E₄*E₅ | E_{b} |
| 459 | E₁ₐEᵢ | | E₄*E₅ | E_{b} |
| 460 | E_{1b}Eᵢ | | E₄*E₅ | E_{b} |
| 461 | E₁ₐEᵢᵢ | | E₄*E₅ | E_{b} |
| 462 | E_{1b}Eᵢᵢ | | E₄*E₅ | E_{b} |
| 463 | E₁ₐEᵢᵢE_{iib} | | E₄*E₅ | E_{b} |
| 464 | E_{1b}EᵢᵢE_{iib} | | E₄*E₅ | E_{b} |
| 465 | E₁ₐE₄ | | E₄*E₅ | E_{b} |
| 466 | E_{1b}E₄ | | E₄*E₅ | E_{b} |
| 467 | E₁ₐEᵢE₄ | | E₄*E₅ | E_{b} |
| 468 | E_{1b}EᵢE₄ | | E₄*E₅ | E_{b} |
| 469 | E₁ₐEᵢᵢE₄ | | E₄*E₅ | E_{b} |
| 470 | E_{1b}EᵢᵢE₄ | | E₄*E₅ | E_{b} |
| 471 | E₁ₐEᵢᵢE_{iib}E₄ | | E₄*E₅ | E_{b} |
| 472 | E_{1b}EᵢᵢE_{iib}E₄ | | E₄*E₅ | E_{b} |
| 473 | E₁ₐEᵢᵢᵢ | | E₄*E₅ | E_{b} |
| 474 | E_{1b}Eᵢᵢᵢ | | E₄*E₅ | E_{b} |
| 475 | E₁ₐEᵢᵢᵢE₄ | | E₄*E₅ | E_{b} |
| 476 | E_{1b}EᵢᵢᵢE₄ | | E₄*E₅ | E_{b} |
| 477 | E₁ₐEᵢᵥ | | E₄*E₅ | E_{b} |
| 478 | E_{1b}Eᵢᵥ | | E₄*E₅ | E_{b} |
| 479 | E₁ₐEᵢᵥE₄ | | E₄*E₅ | E_{b} |
| 480 | E_{1b}EᵢᵥE₄ | | E₄*E₅ | E_{b} |
| 481 | E₁ₐ | AlkL | | E_{b} |
| 482 | E_{1b} | AlkL | | E_{b} |
| 483 | E₁ₐEᵢ | AlkL | | E_{b} |
| 484 | E_{1b}Eᵢ | AlkL | | E_{b} |
| 485 | E₁ₐEᵢᵢ | AlkL | | E_{b} |
| 486 | E_{1b}Eᵢᵢ | AlkL | | E_{b} |
| 487 | E₁ₐEᵢᵢE_{iib} | AlkL | | E_{b} |
| 488 | E_{1b}EᵢᵢE_{iib} | AlkL | | E_{b} |
| 489 | E₁ₐE₄ | AlkL | | E_{b} |
| 490 | E_{1b}E₄ | AlkL | | E_{b} |
| 491 | E₁ₐEᵢE₄ | AlkL | | E_{b} |
| 492 | E_{1b}EᵢE₄ | AlkL | | E_{b} |
| 493 | E₁ₐEᵢᵢE₄ | AlkL | | E_{b} |
| 494 | E_{1b}EᵢᵢE₄ | AlkL | | E_{b} |
| 495 | E₁ₐEᵢᵢE_{iib}E₄ | AlkL | | E_{b} |
| 496 | E_{1b}EᵢᵢE_{iib}E₄ | AlkL | | E_{b} |
| 497 | E₁ₐEᵢᵢᵢ | AlkL | | E_{b} |
| 498 | E_{1b}Eᵢᵢᵢ | AlkL | | E_{b} |
| 499 | E₁ₐEᵢᵢᵢE₄ | AlkL | | E_{b} |
| 500 | E_{1b}EᵢᵢᵢE₄ | AlkL | | E_{b} |
| 501 | E₁ₐEᵢᵥ | AlkL | | E_{b} |
| 502 | E_{1b}Eᵢᵥ | AlkL | | E_{b} |
| 503 | E₁ₐEᵢᵥE₄ | AlkL | | E_{b} |
| 504 | E_{1b}EᵢᵥE₄ | AlkL | | E_{b} |
| 505 | E₁ₐ | AlkL | E₄* | E_{b} |
| 506 | E_{1b} | AlkL | E₄* | E_{b} |
| 507 | E₁ₐEᵢ | AlkL | E₄* | E_{b} |
| 508 | E_{1b}Eᵢ | AlkL | E₄* | E_{b} |
| 509 | E₁ₐEᵢᵢ | AlkL | E₄* | E_{b} |
| 510 | E_{1b}Eᵢᵢ | AlkL | E₄* | E_{b} |
| 511 | E₁ₐEᵢᵢE_{iib} | AlkL | E₄* | E_{b} |
| 512 | E_{1b}EᵢᵢE_{iib} | AlkL | E₄* | E_{b} |
| 513 | E₁ₐE₄ | AlkL | E₄* | E_{b} |
| 514 | E_{1b}E₄ | AlkL | E₄* | E_{b} |
| 515 | E₁ₐEᵢE₄ | AlkL | E₄* | E_{b} |
| 516 | E_{1b}EᵢE₄ | AlkL | E₄* | E_{b} |
| 517 | E₁ₐEᵢᵢE₄ | AlkL | E₄* | E_{b} |
| 518 | E_{1b}EᵢᵢE₄ | AlkL | E₄* | E_{b} |
| 519 | E₁ₐEᵢᵢE_{iib}E₄ | AlkL | E₄* | E_{b} |
| 520 | E_{1b}EᵢᵢE_{iib}E₄ | AlkL | E₄* | E_{b} |
| 521 | E₁ₐEᵢᵢᵢ | AlkL | E₄* | E_{b} |
| 522 | E_{1b}Eᵢᵢᵢ | AlkL | E₄* | E_{b} |
| 523 | E₁ₐEᵢᵢᵢE₄ | AlkL | E₄* | E_{b} |
| 524 | E_{1b}EᵢᵢᵢE₄ | AlkL | E₄* | E_{b} |
| 525 | E₁ₐEᵢᵥ | AlkL | E₄* | E_{b} |
| 526 | E_{1b}Eᵢᵥ | AlkL | E₄* | E_{b} |
| 527 | E₁ₐEᵢᵥE₄ | AlkL | E₄* | E_{b} |
| 528 | E_{1b}EᵢᵥE₄ | AlkL | E₄* | E_{b} |
| 529 | E₁ₐ | AlkL | E₅ | E_{b} |
| 530 | E_{1b} | AlkL | E₅ | E_{b} |
| 531 | E₁ₐEᵢ | AlkL | E₅ | E_{b} |
| 532 | E_{1b}Eᵢ | AlkL | E₅ | E_{b} |
| 533 | E₁ₐEᵢᵢ | AlkL | E₅ | E_{b} |
| 534 | E_{1b}Eᵢᵢ | AlkL | E₅ | E_{b} |
| 535 | E₁ₐEᵢᵢE_{iib} | AlkL | E₅ | E_{b} |
| 536 | E_{1b}EᵢᵢE_{iib} | AlkL | E₅ | E_{b} |
| 537 | E₁ₐE₄ | AlkL | E₅ | E_{b} |
| 538 | E_{1b}E₄ | AlkL | E₅ | E_{b} |
| 539 | E₁ₐEᵢE₄ | AlkL | E₅ | E_{b} |
| 540 | E_{1b}EᵢE₄ | AlkL | E₅ | E_{b} |
| 541 | E₁ₐEᵢᵢE₄ | AlkL | E₅ | E_{b} |
| 542 | E_{1b}EᵢᵢE₄ | AlkL | E₅ | E_{b} |
| 543 | E₁ₐEᵢᵢE_{iib}E₄ | AlkL | E₅ | E_{b} |
| 544 | E_{1b}EᵢᵢE_{iib}E₄ | AlkL | E₅ | E_{b} |
| 545 | E₁ₐEᵢᵢᵢ | AlkL | E₅ | E_{b} |
| 546 | E_{1b}Eᵢᵢᵢ | AlkL | E₅ | E_{b} |
| 547 | E₁ₐEᵢᵢᵢE₄ | AlkL | E₅ | E_{b} |
| 548 | E_{1b}EᵢᵢᵢE₄ | AlkL | E₅ | E_{b} |
| 549 | E₁ₐEᵢᵥ | AlkL | E₅ | E_{b} |
| 550 | E_{1b}Eᵢᵥ | AlkL | E₅ | E_{b} |
| 551 | E₁ₐEᵢᵥE₄ | AlkL | E₅ | E_{b} |
| 552 | E_{1b}EᵢᵥE₄ | AlkL | E₅ | E_{b} |
| 553 | E₁ₐ | AlkL | E₄*E₅ | E_{b} |
| 554 | E_{1b} | AlkL | E₄*E₅ | E_{b} |
| 555 | E₁ₐEᵢ | AlkL | E₄*E₅ | E_{b} |
| 556 | E_{1b}Eᵢ | AlkL | E₄*E₅ | E_{b} |
| 557 | E₁ₐEᵢᵢ | AlkL | E₄*E₅ | E_{b} |
| 558 | E_{1b}Eᵢᵢ | AlkL | E₄*E₅ | E_{b} |
| 559 | E₁ₐEᵢᵢE_{iib} | AlkL | E₄*E₅ | E_{b} |
| 560 | E_{1b}EᵢᵢE_{iib} | AlkL | E₄*E₅ | E_{b} |
| 561 | E₁ₐE₄ | AlkL | E₄*E₅ | E_{b} |
| 562 | E_{1b}E₄ | AlkL | E₄*E₅ | E_{b} |
| 563 | E₁ₐEᵢE₄ | AlkL | E₄*E₅ | E_{b} |
| 564 | E_{1b}EᵢE₄ | AlkL | E₄*E₅ | E_{b} |
| 565 | E₁ₐEᵢᵢE₄ | AlkL | E₄*E₅ | E_{b} |
| 566 | E_{1b}EᵢᵢE₄ | AlkL | E₄*E₅ | E_{b} |
| 567 | E₁ₐEᵢᵢE_{iib}E₄ | AlkL | E₄*E₅ | E_{b} |
| 568 | E_{1b}EᵢᵢE_{iib}E₄ | AlkL | E₄*E₅ | E_{b} |
| 569 | E₁ₐEᵢᵢᵢ | AlkL | E₄*E₅ | E_{b} |
| 570 | E_{1b}Eᵢᵢᵢ | AlkL | E₄*E₅ | E_{b} |
| 571 | E₁ₐEᵢᵢᵢE₄ | AlkL | E₄*E₅ | E_{b} |
| 572 | E_{1b}EᵢᵢᵢE₄ | AlkL | E₄*E₅ | E_{b} |
| 573 | E₁ₐEᵢᵥ | AlkL | E₄*E₅ | E_{b} |
| 574 | E_{1b}Eᵢᵥ | AlkL | E₄*E₅ | E_{b} |
| 575 | E₁ₐEᵢᵥE₄ | AlkL | E₄*E₅ | E_{b} |
| 576 | E_{1b}EᵢᵥE₄ | AlkL | E₄*E₅ | E_{b} |
| 577 | E₁ₐ | | | E_{d} |
| 578 | E_{1b} | | | E_{d} |
| 579 | E₁ₐEᵢ | | | E_{d} |
| 580 | E_{1b}Eᵢ | | | E_{d} |
| 581 | E₁ₐEᵢᵢ | | | E_{d} |
| 582 | E_{1b}Eᵢᵢ | | | E_{d} |
| 583 | E₁ₐEᵢᵢE_{iib} | | | E_{d} |
| 584 | E_{1b}EᵢᵢE_{iib} | | | E_{d} |
| 585 | E₁ₐE₄ | | | E_{d} |
| 586 | E_{1b}E₄ | | | E_{d} |
| 587 | E₁ₐEᵢE₄ | | | E_{d} |
| 588 | E_{1b}EᵢE₄ | | | E_{d} |
| 589 | E₁ₐEᵢᵢE₄ | | | E_{d} |
| 590 | E_{1b}EᵢᵢE₄ | | | E_{d} |
| 591 | E₁ₐEᵢᵢE_{iib}E₄ | | | E_{d} |
| 592 | E_{1b}EᵢᵢE_{iib}E₄ | | | E_{d} |
| 593 | E₁ₐEᵢᵢᵢ | | | E_{d} |
| 594 | E_{1b}Eᵢᵢᵢ | | | E_{d} |
| 595 | E₁ₐEᵢᵢᵢE₄ | | | E_{d} |
| 596 | E_{1b}EᵢᵢᵢE₄ | | | E_{d} |
| 597 | E₁ₐEᵢᵥ | | | E_{d} |
| 598 | E_{1b}Eᵢᵥ | | | E_{d} |
| 599 | E₁ₐEᵢᵥE₄ | | | E_{d} |
| 600 | E_{1b}EᵢᵥE₄ | | | E_{d} |
| 601 | E₁ₐ | | E₄* | E_{d} |
| 602 | E_{1b} | | E₄* | E_{d} |
| 603 | E₁ₐEᵢ | | E₄* | E_{d} |
| 604 | E_{1b}Eᵢ | | E₄* | E_{d} |
| 605 | E₁ₐEᵢᵢ | | E₄* | E_{d} |
| 606 | E_{1b}Eᵢᵢ | | E₄* | E_{d} |
| 607 | E₁ₐEᵢᵢE_{iib} | | E₄* | E_{d} |
| 608 | E_{1b}EᵢᵢE_{iib} | | E₄* | E_{d} |
| 609 | E₁ₐE₄ | | E₄* | E_{d} |
| 610 | E_{1b}E₄ | | E₄* | E_{d} |
| 611 | E₁ₐEᵢE₄ | | E₄* | E_{d} |
| 612 | E_{1b}EᵢE₄ | | E₄* | E_{d} |
| 613 | E₁ₐEᵢᵢE₄ | | E₄* | E_{d} |
| 614 | E_{1b}EᵢᵢE₄ | | E₄* | E_{d} |
| 615 | E₁ₐEᵢᵢE_{iib}E₄ | | E₄* | E_{d} |
| 616 | E_{1b}EᵢᵢE_{iib}E₄ | | E₄* | E_{d} |
| 617 | E₁ₐEᵢᵢᵢ | | E₄* | E_{d} |
| 618 | E_{1b}Eᵢᵢᵢ | | E₄* | E_{d} |
| 619 | E₁ₐEᵢᵢᵢE₄ | | E₄* | E_{d} |
| 620 | E_{1b}EᵢᵢᵢE₄ | | E₄* | E_{d} |
| 621 | E₁ₐEᵢᵥ | | E₄* | E_{d} |
| 622 | E_{1b}Eᵢᵥ | | E₄* | E_{d} |
| 623 | E₁ₐEᵢᵥE₄ | | E₄* | E_{d} |
| 624 | E_{1b}EᵢᵥE₄ | | E₄* | E_{d} |
| 625 | E₁ₐ | | E₅ | E_{d} |
| 626 | E_{1b} | | E₅ | E_{d} |
| 627 | E₁ₐEᵢ | | E₅ | E_{d} |
| 628 | E_{1b}Eᵢ | | E₅ | E_{d} |
| 629 | E₁ₐEᵢᵢ | | E₅ | E_{d} |
| 630 | E_{1b}Eᵢᵢ | | E₅ | E_{d} |
| 631 | E₁ₐEᵢᵢE_{iib} | | E₅ | E_{d} |
| 632 | E_{1b}EᵢᵢE_{iib} | | E₅ | E_{d} |
| 633 | E₁ₐE₄ | | E₅ | E_{d} |
| 634 | E_{1b}E₄ | | E₅ | E_{d} |
| 635 | E₁ₐEᵢE₄ | | E₅ | E_{d} |
| 636 | E_{1b}EᵢE₄ | | E₅ | E_{d} |
| 637 | E₁ₐEᵢᵢE₄ | | E₅ | E_{d} |
| 638 | E_{1b}EᵢᵢE₄ | | E₅ | E_{d} |
| 639 | E₁ₐEᵢᵢE_{iib}E₄ | | E₅ | E_{d} |
| 640 | E_{1b}EᵢᵢE_{iib}E₄ | | E₅ | E_{d} |
| 641 | E₁ₐEᵢᵢᵢ | | E₅ | E_{d} |
| 642 | E_{1b}Eᵢᵢᵢ | | E₅ | E_{d} |
| 643 | E₁ₐEᵢᵢᵢE₄ | | E₅ | E_{d} |
| 644 | E_{1b}EᵢᵢᵢE₄ | | E₅ | E_{d} |
| 645 | E₁ₐEᵢᵥ | | E₅ | E_{d} |
| 646 | E_{1b}Eᵢᵥ | | E₅ | E_{d} |
| 647 | E₁ₐEᵢᵥE₄ | | E₅ | E_{d} |
| 648 | E_{1b}EᵢᵥE₄ | | E₅ | E_{d} |
| 649 | E₁ₐ | | E₄*E₅ | E_{d} |
| 650 | E_{1b} | | E₄*E₅ | E_{d} |
| 651 | E₁ₐEᵢ | | E₄*E₅ | E_{d} |
| 652 | E_{1b}Eᵢ | | E₄*E₅ | E_{d} |
| 653 | E₁ₐEᵢᵢ | | E₄*E₅ | E_{d} |
| 654 | E_{1b}Eᵢᵢ | | E₄*E₅ | E_{d} |
| 655 | E₁ₐEᵢᵢE_{iib} | | E₄*E₅ | E_{d} |
| 656 | E_{1b}EᵢᵢE_{iib} | | E₄*E₅ | E_{d} |
| 657 | E₁ₐE₄ | | E₄*E₅ | E_{d} |
| 658 | E_{1b}E₄ | | E₄*E₅ | E_{d} |
| 659 | E₁ₐEᵢE₄ | | E₄*E₅ | E_{d} |
| 660 | E_{1b}EᵢE₄ | | E₄*E₅ | E_{d} |
| 661 | E₁ₐEᵢᵢE₄ | | E₄*E₅ | E_{d} |
| 662 | E_{1b}EᵢᵢE₄ | | E₄*E₅ | E_{d} |
| 663 | E₁ₐEᵢᵢE_{iib}E₄ | | E₄*E₅ | E_{d} |
| 664 | E_{1b}EᵢᵢE_{iib}E₄ | | E₄*E₅ | E_{d} |
| 665 | E₁ₐEᵢᵢᵢ | | E₄*E₅ | E_{d} |
| 666 | E_{1b}Eᵢᵢᵢ | | E₄*E₅ | E_{d} |
| 667 | E₁ₐEᵢᵢᵢE₄ | | E₄*E₅ | E_{d} |
| 668 | E_{1b}EᵢᵢᵢE₄ | | E₄*E₅ | E_{d} |
| 669 | E₁ₐEᵢᵥ | | E₄*E₅ | E_{d} |
| 670 | E_{1b}Eᵢᵥ | | E₄*E₅ | E_{d} |
| 671 | E₁ₐEᵢᵥE₄ | | E₄*E₅ | E_{d} |
| 672 | E_{1b}EᵢᵥE₄ | | E₄*E₅ | E_{d} |
| 673 | E₁ₐ | AlkL | | E_{d} |
| 674 | E_{1b} | AlkL | | E_{d} |
| 675 | E₁ₐEᵢ | AlkL | | E_{d} |
| 676 | E_{1b}Eᵢ | AlkL | | E_{d} |
| 677 | E₁ₐEᵢᵢ | AlkL | | E_{d} |
| 678 | E_{1b}Eᵢᵢ | AlkL | | E_{d} |
| 679 | E₁ₐEᵢᵢE_{iib} | AlkL | | E_{d} |
| 680 | E_{1b}EᵢᵢE_{iib} | AlkL | | E_{d} |
| 681 | E₁ₐE₄ | AlkL | | E_{d} |
| 682 | E_{1b}E₄ | AlkL | | E_{d} |
| 683 | E₁ₐEᵢE₄ | AlkL | | E_{d} |
| 684 | E_{1b}EᵢE₄ | AlkL | | E_{d} |
| 685 | E₁ₐEᵢᵢE₄ | AlkL | | E_{d} |
| 686 | E_{1b}EᵢᵢE₄ | AlkL | | E_{d} |
| 687 | E₁ₐEᵢᵢE_{iib}E₄ | AlkL | | E_{d} |
| 688 | E_{1b}EᵢᵢE_{iib}E₄ | AlkL | | E_{d} |
| 689 | E₁ₐEᵢᵢᵢ | AlkL | | E_{d} |
| 690 | E_{1b}Eᵢᵢᵢ | AlkL | | E_{d} |
| 691 | E₁ₐEᵢᵢᵢE₄ | AlkL | | E_{d} |
| 692 | E_{1b}EᵢᵢᵢE₄ | AlkL | | E_{d} |
| 693 | E₁ₐEᵢᵥ | AlkL | | E_{d} |
| 694 | E_{1b}Eᵢᵥ | AlkL | | E_{d} |
| 695 | E₁ₐEᵢᵥE₄ | AlkL | | E_{d} |
| 696 | E_{1b}EᵢᵥE₄ | AlkL | | E_{d} |
| 697 | E₁ₐ | AlkL | E₄* | E_{d} |
| 698 | E_{1b} | AlkL | E₄* | E_{d} |
| 699 | E₁ₐEᵢ | AlkL | E₄* | E_{d} |
| 700 | E_{1b}Eᵢ | AlkL | E₄* | E_{d} |
| 701 | E₁ₐEᵢᵢ | AlkL | E₄* | E_{d} |
| 702 | E_{1b}Eᵢᵢ | AlkL | E₄* | E_{d} |
| 703 | E₁ₐEᵢᵢE_{iib} | AlkL | E₄* | E_{d} |
| 704 | E_{1b}EᵢᵢE_{iib} | AlkL | E₄* | E_{d} |
| 705 | E₁ₐE₄ | AlkL | E₄* | E_{d} |
| 706 | E_{1b}E₄ | AlkL | E₄* | E_{d} |
| 707 | E₁ₐEᵢE₄ | AlkL | E₄* | E_{d} |
| 708 | E_{1b}EᵢE₄ | AlkL | E₄* | E_{d} |
| 709 | E₁ₐEᵢᵢE₄ | AlkL | E₄* | E_{d} |
| 710 | E_{1b}EᵢᵢE₄ | AlkL | E₄* | E_{d} |
| 711 | E₁ₐEᵢᵢE_{iib}E₄ | AlkL | E₄* | E_{d} |
| 712 | E_{1b}EᵢᵢE_{iib}E₄ | AlkL | E₄* | E_{d} |
| 713 | E₁ₐEᵢᵢᵢ | AlkL | E₄* | E_{d} |
| 714 | E_{1b}Eᵢᵢᵢ | AlkL | E₄* | E_{d} |
| 715 | E₁ₐEᵢᵢᵢE₄ | AlkL | E₄* | E_{d} |
| 716 | E_{1b}EᵢᵢᵢE₄ | AlkL | E₄* | E_{d} |
| 717 | E₁ₐEᵢᵥ | AlkL | E₄* | E_{d} |
| 718 | E_{1b}Eᵢᵥ | AlkL | E₄* | E_{d} |
| 719 | E₁ₐEᵢᵥE₄ | AlkL | E₄* | E_{d} |
| 720 | E_{1b}EᵢᵥE₄ | AlkL | E₄* | E_{d} |
| 721 | E₁ₐ | AlkL | E₅ | E_{d} |
| 722 | E_{1b} | AlkL | E₅ | E_{d} |
| 723 | E₁ₐEᵢ | AlkL | E₅ | E_{d} |
| 724 | E_{1b}Eᵢ | AlkL | E₅ | E_{d} |
| 725 | E₁ₐEᵢᵢ | AlkL | E₅ | E_{d} |
| 726 | E_{1b}Eᵢᵢ | AlkL | E₅ | E_{d} |
| 727 | E₁ₐEᵢᵢE_{iib} | AlkL | E₅ | E_{d} |
| 728 | E_{1b}EᵢᵢE_{iib} | AlkL | E₅ | E_{d} |
| 729 | E₁ₐE₄ | AlkL | E₅ | E_{d} |
| 730 | E_{1b}E₄ | AlkL | E₅ | E_{d} |
| 731 | E₁ₐEᵢE₄ | AlkL | E₅ | E_{d} |
| 732 | E_{1b}EᵢE₄ | AlkL | E₅ | E_{d} |
| 733 | E₁ₐEᵢᵢE₄ | AlkL | E₅ | E_{d} |
| 734 | E_{1b}EᵢᵢE₄ | AlkL | E₅ | E_{d} |
| 735 | E₁ₐEᵢᵢE_{iib}E₄ | AlkL | E₅ | E_{d} |
| 736 | E_{1b}EᵢᵢE_{iib}E₄ | AlkL | E₅ | E_{d} |
| 737 | E₁ₐEᵢᵢᵢ | AlkL | E₅ | E_{d} |
| 738 | E_{1b}Eᵢᵢᵢ | AlkL | E₅ | E_{d} |
| 739 | E₁ₐEᵢᵢᵢE₄ | AlkL | E₅ | E_{d} |
| 740 | E_{1b}EᵢᵢᵢE₄ | AlkL | E₅ | E_{d} |
| 741 | E₁ₐEᵢᵥ | AlkL | E₅ | E_{d} |
| 742 | E_{1b}Eᵢᵥ | AlkL | E₅ | E_{d} |
| 743 | E₁ₐEᵢᵥE₄ | AlkL | E₅ | E_{d} |
| 744 | E_{1b}EᵢᵥE₄ | AlkL | E₅ | E_{d} |
| 745 | E₁ₐ | AlkL | E₄*E₅ | E_{d} |
| 746 | E_{1b} | AlkL | E₄*E₅ | E_{d} |
| 747 | E₁ₐEᵢ | AlkL | E₄*E₅ | E_{d} |
| 748 | E_{1b}Eᵢ | AlkL | E₄*E₅ | E_{d} |
| 749 | E₁ₐEᵢᵢ | AlkL | E₄*E₅ | E_{d} |
| 750 | E_{1b}Eᵢᵢ | AlkL | E₄*E₅ | E_{d} |
| 751 | E₁ₐEᵢᵢE_{iib} | AlkL | E₄*E₅ | E_{d} |
| 752 | E_{1b}EᵢᵢE_{iib} | AlkL | E₄*E₅ | E_{d} |
| 753 | E₁ₐE₄ | AlkL | E₄*E₅ | E_{d} |
| 754 | E_{1b}E₄ | AlkL | E₄*E₅ | E_{d} |
| 755 | E₁ₐEᵢE₄ | AlkL | E₄*E₅ | E_{d} |
| 756 | E_{1b}EᵢE₄ | AlkL | E₄*E₅ | E_{d} |
| 757 | E₁ₐEᵢᵢE₄ | AlkL | E₄*E₅ | E_{d} |
| 758 | E_{1b}EᵢᵢE₄ | AlkL | E₄*E₅ | E_{d} |
| 759 | E₁ₐEᵢᵢE_{iib}E₄ | AlkL | E₄*E₅ | E_{d} |
| 760 | E_{1b}EᵢᵢE_{iib}E₄ | AlkL | E₄*E₅ | E_{d} |
| 761 | E₁ₐEᵢᵢᵢ | AlkL | E₄*E₅ | E_{d} |
| 762 | E_{1b}Eᵢᵢᵢ | AlkL | E₄*E₅ | E_{d} |
| 763 | E₁ₐEᵢᵢᵢE₄ | AlkL | E₄*E₅ | E_{d} |
| 764 | E_{1b}EᵢᵢᵢE₄ | AlkL | E₄*E₅ | E_{d} |
| 765 | E₁ₐEᵢᵥ | AlkL | E₄*E₅ | E_{d} |
| 766 | E_{1b}Eᵢᵥ | AlkL | E₄*E₅ | E_{d} |
| 767 | E₁ₐEᵢᵥE₄ | AlkL | E₄*E₅ | E_{d} |
| 768 | E_{1b}EᵢᵥE₄ | AlkL | E₄*E₅ | E_{d} |
| 769 | E₁ₐ | | | Eₑ |
| 770 | E_{1b} | | | Eₑ |
| 771 | E₁ₐEᵢ | | | Eₑ |
| 772 | E_{1b}Eᵢ | | | Eₑ |
| 773 | E₁ₐEᵢᵢ | | | Eₑ |
| 774 | E_{1b}Eᵢᵢ | | | Eₑ |
| 775 | E₁ₐEᵢᵢE_{iib} | | | Eₑ |
| 776 | E_{1b}EᵢᵢE_{iib} | | | Eₑ |
| 777 | E₁ₐE₄ | | | Eₑ |
| 778 | E_{1b}E₄ | | | Eₑ |
| 779 | E₁ₐEᵢE₄ | | | Eₑ |
| 780 | E_{1b}EᵢE₄ | | | Eₑ |
| 781 | E₁ₐEᵢᵢE₄ | | | Eₑ |
| 782 | E_{1b}EᵢᵢE₄ | | | Eₑ |
| 783 | E₁ₐEᵢᵢE_{iib}E₄ | | | Eₑ |
| 784 | E_{1b}EᵢᵢE_{iib}E₄ | | | Eₑ |
| 785 | E₁ₐEᵢᵢᵢ | | | Eₑ |
| 786 | E_{1b}Eᵢᵢᵢ | | | Eₑ |
| 787 | E₁ₐEᵢᵢᵢE₄ | | | Eₑ |
| 788 | E_{1b}EᵢᵢᵢE₄ | | | Eₑ |
| 789 | E₁ₐEᵢᵥ | | | Eₑ |
| 790 | E_{1b}Eᵢᵥ | | | Eₑ |
| 791 | E₁ₐEᵢᵥE₄ | | | Eₑ |
| 792 | E_{1b}EᵢᵥE₄ | | | Eₑ |
| 793 | E₁ₐ | | E₄* | Eₑ |
| 794 | E_{1b} | | E₄* | Eₑ |
| 795 | E₁ₐEᵢ | | E₄* | Eₑ |
| 796 | E_{1b}Eᵢ | | E₄* | Eₑ |
| 797 | E₁ₐEᵢᵢ | | E₄* | Eₑ |
| 798 | E_{1b}Eᵢᵢ | | E₄* | Eₑ |
| 799 | E₁ₐEᵢᵢE_{iib} | | E₄* | Eₑ |
| 800 | E_{1b}EᵢᵢE_{iib} | | E₄* | Eₑ |
| 801 | E₁ₐE₄ | | E₄* | Eₑ |
| 802 | E_{1b}E₄ | | E₄* | Eₑ |
| 803 | E₁ₐEᵢE₄ | | E₄* | Eₑ |
| 804 | E_{1b}EᵢE₄ | | E₄* | Eₑ |
| 805 | E₁ₐEᵢᵢE₄ | | E₄* | Eₑ |
| 806 | E_{1b}EᵢᵢE₄ | | E₄* | Eₑ |
| 807 | E₁ₐEᵢᵢE_{iib}E₄ | | E₄* | Eₑ |
| 808 | E_{1b}EᵢᵢE_{iib}E₄ | | E₄* | Eₑ |
| 809 | E₁ₐEᵢᵢᵢ | | E₄* | Eₑ |
| 810 | E_{1b}Eᵢᵢᵢ | | E₄* | Eₑ |
| 811 | E₁ₐEᵢᵢᵢE₄ | | E₄* | Eₑ |
| 812 | E_{1b}EᵢᵢᵢE₄ | | E₄* | Eₑ |
| 813 | E₁ₐEᵢᵥ | | E₄* | Eₑ |
| 814 | E_{1b}Eᵢᵥ | | E₄* | Eₑ |
| 815 | E₁ₐEᵢᵥE₄ | | E₄* | Eₑ |
| 816 | E_{1b}EᵢᵥE₄ | | E₄* | Eₑ |
| 817 | E₁ₐ | | E₅ | Eₑ |
| 818 | E_{1b} | | E₅ | Eₑ |
| 819 | E₁ₐEᵢ | | E₅ | Eₑ |
| 820 | E_{1b}Eᵢ | | E₅ | Eₑ |
| 821 | E₁ₐEᵢᵢ | | E₅ | Eₑ |
| 822 | E_{1b}Eᵢᵢ | | E₅ | Eₑ |
| 823 | E₁ₐEᵢᵢE_{iib} | | E₅ | Eₑ |
| 824 | E_{1b}EᵢᵢE_{iib} | | E₅ | Eₑ |
| 825 | E₁ₐE₄ | | E₅ | Eₑ |
| 826 | E_{1b}E₄ | | E₅ | Eₑ |
| 827 | E₁ₐEᵢE₄ | | E₅ | Eₑ |
| 828 | E_{1b}EᵢE₄ | | E₅ | Eₑ |
| 829 | E₁ₐEᵢᵢE₄ | | E₅ | Eₑ |
| 830 | E_{1b}EᵢᵢE₄ | | E₅ | Eₑ |
| 831 | E₁ₐEᵢᵢE_{iib}E₄ | | E₅ | Eₑ |
| 832 | E_{1b}EᵢᵢE_{iib}E₄ | | E₅ | Eₑ |
| 833 | E₁ₐEᵢᵢᵢ | | E₅ | Eₑ |
| 834 | E_{1b}Eᵢᵢᵢ | | E₅ | Eₑ |
| 835 | E₁ₐEᵢᵢᵢE₄ | | E₅ | Eₑ |
| 836 | E_{1b}EᵢᵢᵢE₄ | | E₅ | Eₑ |
| 837 | E₁ₐEᵢᵥ | | E₅ | Eₑ |
| 838 | E_{1b}Eᵢᵥ | | E₅ | Eₑ |
| 839 | E₁ₐEᵢᵥE₄ | | E₅ | Eₑ |
| 840 | E_{1b}EᵢᵥE₄ | | E₅ | Eₑ |
| 841 | E₁ₐ | | E₄*E₅ | Eₑ |
| 842 | E_{1b} | | E₄*E₅ | Eₑ |
| 843 | E₁ₐEᵢ | | E₄*E₅ | Eₑ |
| 844 | E_{1b}Eᵢ | | E₄*E₅ | Eₑ |
| 845 | E₁ₐEᵢᵢ | | E₄*E₅ | Eₑ |
| 846 | E_{1b}Eᵢᵢ | | E₄*E₅ | Eₑ |
| 847 | E₁ₐEᵢᵢE_{iib} | | E₄*E₅ | Eₑ |
| 848 | E_{1b}EᵢᵢE_{iib} | | E₄*E₅ | Eₑ |
| 849 | E₁ₐE₄ | | E₄*E₅ | Eₑ |
| 850 | E_{1b}E₄ | | E₄*E₅ | Eₑ |
| 851 | E₁ₐEᵢE₄ | | E₄*E₅ | Eₑ |
| 852 | E_{1b}EᵢE₄ | | E₄*E₅ | Eₑ |
| 853 | E₁ₐEᵢᵢE₄ | | E₄*E₅ | Eₑ |
| 854 | E_{1b}EᵢᵢE₄ | | E₄*E₅ | Eₑ |
| 855 | E₁ₐEᵢᵢE_{iib}E₄ | | E₄*E₅ | Eₑ |
| 856 | E_{1b}EᵢᵢE_{iib}E₄ | | E₄*E₅ | Eₑ |
| 857 | E₁ₐEᵢᵢᵢ | | E₄*E₅ | Eₑ |
| 858 | E_{1b}Eᵢᵢᵢ | | E₄*E₅ | Eₑ |
| 859 | E₁ₐEᵢᵢᵢE₄ | | E₄*E₅ | Eₑ |
| 860 | E_{1b}EᵢᵢᵢE₄ | | E₄*E₅ | Eₑ |
| 861 | E₁ₐEᵢᵥ | | E₄*E₅ | Eₑ |
| 862 | E_{1b}Eᵢᵥ | | E₄*E₅ | Eₑ |
| 863 | E₁ₐEᵢᵥE₄ | | E₄*E₅ | Eₑ |
| 864 | E_{1b}EᵢᵥE₄ | | E₄*E₅ | Eₑ |
| 865 | E₁ₐ | AlkL | | Eₑ |
| 866 | E_{1b} | AlkL | | Eₑ |
| 867 | E₁ₐEᵢ | AlkL | | Eₑ |
| 868 | E_{1b}Eᵢ | AlkL | | Eₑ |
| 869 | E₁ₐEᵢᵢ | AlkL | | Eₑ |
| 870 | E_{1b}Eᵢᵢ | AlkL | | Eₑ |
| 871 | E₁ₐEᵢᵢE_{iib} | AlkL | | Eₑ |
| 872 | E_{1b}EᵢᵢE_{iib} | AlkL | | Eₑ |
| 873 | E₁ₐE₄ | AlkL | | Eₑ |
| 874 | E_{1b}E₄ | AlkL | | Eₑ |
| 875 | E₁ₐEᵢE₄ | AlkL | | Eₑ |
| 876 | E_{1b}EᵢE₄ | AlkL | | Eₑ |
| 877 | E₁ₐEᵢᵢE₄ | AlkL | | Eₑ |
| 878 | E_{1b}EᵢᵢE₄ | AlkL | | Eₑ |
| 879 | E₁ₐEᵢᵢE_{iib}E₄ | AlkL | | Eₑ |
| 880 | E_{1b}EᵢᵢE_{iib}E₄ | AlkL | | Eₑ |
| 881 | E₁ₐEᵢᵢᵢ | AlkL | | Eₑ |
| 882 | E_{1b}Eᵢᵢᵢ | AlkL | | Eₑ |
| 883 | E₁ₐEᵢᵢᵢE₄ | AlkL | | Eₑ |
| 884 | E_{1b}EᵢᵢᵢE₄ | AlkL | | Eₑ |
| 885 | E₁ₐEᵢᵥ | AlkL | | Eₑ |
| 886 | E_{1b}Eᵢᵥ | AlkL | | Eₑ |
| 887 | E₁ₐEᵢᵥE₄ | AlkL | | Eₑ |
| 888 | E_{1b}EᵢᵥE₄ | AlkL | | Eₑ |
| 889 | E₁ₐ | AlkL | E₄* | Eₑ |
| 890 | E_{1b} | AlkL | E₄* | Eₑ |
| 891 | E₁ₐEᵢ | AlkL | E₄* | Eₑ |
| 892 | E_{1b}Eᵢ | AlkL | E₄* | Eₑ |
| 893 | E₁ₐEᵢᵢ | AlkL | E₄* | Eₑ |
| 894 | E_{1b}Eᵢᵢ | AlkL | E₄* | Eₑ |
| 895 | E₁ₐEᵢᵢE_{iib} | AlkL | E₄* | Eₑ |
| 896 | E_{1b}EᵢᵢE_{iib} | AlkL | E₄* | Eₑ |
| 897 | E₁ₐE₄ | AlkL | E₄* | Eₑ |
| 898 | E_{1b}E₄ | AlkL | E₄* | Eₑ |
| 899 | E₁ₐEᵢE₄ | AlkL | E₄* | Eₑ |
| 900 | E_{1b}EᵢE₄ | AlkL | E₄* | Eₑ |
| 901 | E₁ₐEᵢᵢE₄ | AlkL | E₄* | Eₑ |
| 902 | E_{1b}EᵢᵢE₄ | AlkL | E₄* | Eₑ |
| 903 | E₁ₐEᵢᵢE_{iib}E₄ | AlkL | E₄* | Eₑ |
| 904 | E_{1b}EᵢᵢE_{iib}E₄ | AlkL | E₄* | Eₑ |
| 905 | E₁ₐEᵢᵢᵢ | AlkL | E₄* | Eₑ |
| 906 | E_{1b}Eᵢᵢᵢ | AlkL | E₄* | Eₑ |
| 907 | E₁ₐEᵢᵢᵢE₄ | AlkL | E₄* | Eₑ |
| 908 | E_{1b}EᵢᵢᵢE₄ | AlkL | E₄* | Eₑ |
| 909 | E₁ₐEᵢᵥ | AlkL | E₄* | Eₑ |
| 910 | E_{1b}Eᵢᵥ | AlkL | E₄* | Eₑ |
| 911 | E₁ₐEᵢᵥE₄ | AlkL | E₄* | Eₑ |
| 912 | E_{1b}EᵢᵥE₄ | AlkL | E₄* | Eₑ |
| 913 | E₁ₐ | AlkL | E₅ | Eₑ |
| 914 | E_{1b} | AlkL | E₅ | Eₑ |
| 915 | E₁ₐEᵢ | AlkL | E₅ | Eₑ |
| 916 | E_{1b}Eᵢ | AlkL | E₅ | Eₑ |
| 917 | E₁ₐEᵢᵢ | AlkL | E₅ | Eₑ |
| 918 | E_{1b}Eᵢᵢ | AlkL | E₅ | Eₑ |
| 919 | E₁ₐEᵢᵢE_{iib} | AlkL | E₅ | Eₑ |
| 920 | E_{1b}EᵢᵢE_{iib} | AlkL | E₅ | Eₑ |
| 921 | E₁ₐE₄ | AlkL | E₅ | Eₑ |
| 922 | E_{1b}E₄ | AlkL | E₅ | Eₑ |
| 923 | E₁ₐEᵢE₄ | AlkL | E₅ | Eₑ |
| 924 | E_{1b}EᵢE₄ | AlkL | E₅ | Eₑ |
| 925 | E₁ₐEᵢᵢE₄ | AlkL | E₅ | Eₑ |
| 926 | E_{1b}EᵢᵢE₄ | AlkL | E₅ | Eₑ |
| 927 | E₁ₐEᵢᵢE_{iib}E₄ | AlkL | E₅ | Eₑ |
| 928 | E_{1b}EᵢᵢE_{iib}E₄ | AlkL | E₅ | Eₑ |
| 929 | E₁ₐEᵢᵢᵢ | AlkL | E₅ | Eₑ |
| 930 | E_{1b}Eᵢᵢᵢ | AlkL | E₅ | Eₑ |
| 931 | E₁ₐEᵢᵢᵢE₄ | AlkL | E₅ | Eₑ |
| 932 | E_{1b}EᵢᵢᵢE₄ | AlkL | E₅ | Eₑ |
| 933 | E₁ₐEᵢᵥ | AlkL | E₅ | Eₑ |
| 934 | E_{1b}Eᵢᵥ | AlkL | E₅ | Eₑ |
| 935 | E₁ₐEᵢᵥE₄ | AlkL | E₅ | Eₑ |
| 936 | E_{1b}EᵢᵥE₄ | AlkL | E₅ | Eₑ |
| 937 | E₁ₐ | AlkL | E₄*E₅ | Eₑ |
| 938 | E_{1b} | AlkL | E₄*E₅ | Eₑ |
| 939 | E₁ₐEᵢ | AlkL | E₄*E₅ | Eₑ |
| 940 | E_{1b}Eᵢ | AlkL | E₄*E₅ | Eₑ |
| 941 | E₁ₐEᵢᵢ | AlkL | E₄*E₅ | Eₑ |
| 942 | E_{1b}Eᵢᵢ | AlkL | E₄*E₅ | Eₑ |
| 943 | E₁ₐEᵢᵢE_{iib} | AlkL | E₄*E₅ | Eₑ |
| 944 | E_{1b}EᵢᵢE_{iib} | AlkL | E₄*E₅ | Eₑ |
| 945 | E₁ₐE₄ | AlkL | E₄*E₅ | Eₑ |
| 946 | E_{1b}E₄ | AlkL | E₄*E₅ | Eₑ |
| 947 | E₁ₐEᵢE₄ | AlkL | E₄*E₅ | Eₑ |
| 948 | E_{1b}EᵢE₄ | AlkL | E₄*E₅ | Eₑ |
| 949 | E₁ₐEᵢᵢE₄ | AlkL | E₄*E₅ | Eₑ |
| 950 | E_{1b}EᵢᵢE₄ | AlkL | E₄*E₅ | Eₑ |
| 951 | E₁ₐEᵢᵢE_{iib}E₄ | AlkL | E₄*E₅ | Eₑ |
| 952 | E_{1b}EᵢᵢE_{iib}E₄ | AlkL | E₄*E₅ | Eₑ |
| 953 | E₁ₐEᵢᵢᵢ | AlkL | E₄*E₅ | Eₑ |
| 954 | E_{1b}Eᵢᵢᵢ | AlkL | E₄*E₅ | Eₑ |
| 955 | E₁ₐEᵢᵢᵢE₄ | AlkL | E₄*E₅ | Eₑ |
| 956 | E_{1b}EᵢᵢᵢE₄ | AlkL | E₄*E₅ | Eₑ |
| 957 | E₁ₐEᵢᵥ | AlkL | E₄*E₅ | Eₑ |
| 958 | E_{1b}Eᵢᵥ | AlkL | E₄*E₅ | Eₑ |
| 959 | E₁ₐEᵢᵥE₄ | AlkL | E₄*E₅ | Eₑ |
| 960 | E_{1b}EᵢᵥE₄ | AlkL | E₄*E₅ | Eₑ |
| 961 | E₁ₐ | | | E_{f} |
| 962 | E_{1b} | | | E_{f} |
| 963 | E₁ₐEᵢ | | | E_{f} |
| 964 | E_{1b}Eᵢ | | | E_{f} |
| 965 | E₁ₐEᵢᵢ | | | E_{f} |
| 966 | E_{1b}Eᵢᵢ | | | E_{f} |
| 967 | E₁ₐEᵢᵢE_{iib} | | | E_{f} |
| 968 | E_{1b}EᵢᵢE_{iib} | | | E_{f} |
| 969 | E₁ₐE₄ | | | E_{f} |
| 970 | E_{1b}E₄ | | | E_{f} |
| 971 | E₁ₐEᵢE₄ | | | E_{f} |
| 972 | E_{1b}EᵢE₄ | | | E_{f} |
| 973 | E₁ₐEᵢᵢE₄ | | | E_{f} |
| 974 | E_{1b}EᵢᵢE₄ | | | E_{f} |
| 975 | E₁ₐEᵢᵢE_{iib}E₄ | | | E_{f} |
| 976 | E_{1b}EᵢᵢE_{iib}E₄ | | | E_{f} |
| 977 | E₁ₐEᵢᵢᵢ | | | E_{f} |
| 978 | E_{1b}Eᵢᵢᵢ | | | E_{f} |
| 979 | E₁ₐEᵢᵢᵢE₄ | | | E_{f} |
| 980 | E_{1b}EᵢᵢᵢE₄ | | | E_{f} |
| 981 | E₁ₐEᵢᵥ | | | E_{f} |
| 982 | E_{1b}Eᵢᵥ | | | E_{f} |
| 983 | E₁ₐEᵢᵥE₄ | | | E_{f} |
| 984 | E_{1b}EᵢᵥE₄ | | | E_{f} |
| 985 | E₁ₐ | | E₄* | E_{f} |
| 986 | E_{1b} | | E₄* | E_{f} |
| 987 | E₁ₐEᵢ | | E₄* | E_{f} |
| 988 | E_{1b}Eᵢ | | E₄* | E_{f} |
| 989 | E₁ₐEᵢᵢ | | E₄* | E_{f} |
| 990 | E_{1b}Eᵢᵢ | | E₄* | E_{f} |
| 991 | E₁ₐEᵢᵢE_{iib} | | E₄* | E_{f} |
| 992 | E_{1b}EᵢᵢE_{iib} | | E₄* | E_{f} |
| 993 | E₁ₐE₄ | | E₄* | E_{f} |
| 994 | E_{1b}E₄ | | E₄* | E_{f} |
| 995 | E₁ₐEᵢE₄ | | E₄* | E_{f} |
| 996 | E_{1b}EᵢE₄ | | E₄* | E_{f} |
| 997 | E₁ₐEᵢᵢE₄ | | E₄* | E_{f} |
| 998 | E_{1b}EᵢᵢE₄ | | E₄* | E_{f} |
| 999 | E₁ₐEᵢᵢE_{iib}E₄ | | E₄* | E_{f} |
| 1000 | E_{1b}EᵢᵢE_{iib}E₄ | | E₄* | E_{f} |
| 1001 | E₁ₐEᵢᵢᵢ | | E₄* | E_{f} |
| 1002 | E_{1b}Eᵢᵢᵢ | | E₄* | E_{f} |
| 1003 | E₁ₐEᵢᵢᵢE₄ | | E₄* | E_{f} |
| 1004 | E_{1b}EᵢᵢᵢE₄ | | E₄* | E_{f} |
| 1005 | E₁ₐEᵢᵥ | | E₄* | E_{f} |
| 1006 | E_{1b}Eᵢᵥ | | E₄* | E_{f} |
| 1007 | E₁ₐEᵢᵥE₄ | | E₄* | E_{f} |
| 1008 | E_{1b}EᵢᵥE₄ | | E₄* | E_{f} |
| 1009 | E₁ₐ | | E₅ | E_{f} |
| 1010 | E_{1b} | | E₅ | E_{f} |
| 1011 | E₁ₐEᵢ | | E₅ | E_{f} |
| 1012 | E_{1b}Eᵢ | | E₅ | E_{f} |
| 1013 | E₁ₐEᵢᵢ | | E₅ | E_{f} |
| 1014 | E_{1b}Eᵢᵢ | | E₅ | E_{f} |
| 1015 | E₁ₐEᵢᵢE_{iib} | | E₅ | E_{f} |
| 1016 | E_{1b}EᵢᵢE_{iib} | | E₅ | E_{f} |
| 1017 | E₁ₐE₄ | | E₅ | E_{f} |
| 1018 | E_{1b}E₄ | | E₅ | E_{f} |
| 1019 | E₁ₐEᵢE₄ | | E₅ | E_{f} |
| 1020 | E_{1b}EᵢE₄ | | E₅ | E_{f} |
| 1021 | E₁ₐEᵢᵢE₄ | | E₅ | E_{f} |
| 1022 | E_{1b}EᵢᵢE₄ | | E₅ | E_{f} |
| 1023 | E₁ₐEᵢᵢE_{iib}E₄ | | E₅ | E_{f} |
| 1024 | E_{1b}EᵢᵢE_{iib}E₄ | | E₅ | E_{f} |
| 1025 | E₁ₐEᵢᵢᵢ | | E₅ | E_{f} |
| 1026 | E_{1b}Eᵢᵢᵢ | | E₅ | E_{f} |
| 1027 | E₁ₐEᵢᵢᵢE₄ | | E₅ | E_{f} |
| 1028 | E_{1b}EᵢᵢᵢE₄ | | E₅ | E_{f} |
| 1029 | E₁ₐEᵢᵥ | | E₅ | E_{f} |
| 1030 | E_{1b}Eᵢᵥ | | E₅ | E_{f} |
| 1031 | E₁ₐEᵢᵥE₄ | | E₅ | E_{f} |
| 1032 | E_{1b}EᵢᵥE₄ | | E₅ | E_{f} |
| 1033 | E₁ₐ | | E₄*E₅ | E_{f} |
| 1034 | E_{1b} | | E₄*E₅ | E_{f} |
| 1035 | E₁ₐEᵢ | | E₄*E₅ | E_{f} |
| 1036 | E_{1b}Eᵢ | | E₄*E₅ | E_{f} |
| 1037 | E₁ₐEᵢᵢ | | E₄*E₅ | E_{f} |
| 1038 | E_{1b}Eᵢᵢ | | E₄*E₅ | E_{f} |
| 1039 | E₁ₐEᵢᵢE_{iib} | | E₄*E₅ | E_{f} |
| 1040 | E_{1b}EᵢᵢE_{iib} | | E₄*E₅ | E_{f} |
| 1041 | E₁ₐE₄ | | E₄*E₅ | E_{f} |
| 1042 | E_{1b}E₄ | | E₄*E₅ | E_{f} |
| 1043 | E₁ₐEᵢE₄ | | E₄*E₅ | E_{f} |
| 1044 | E_{1b}EᵢE₄ | | E₄*E₅ | E_{f} |
| 1045 | E₁ₐEᵢᵢE₄ | | E₄*E₅ | E_{f} |
| 1046 | E_{1b}EᵢᵢE₄ | | E₄*E₅ | E_{f} |
| 1047 | E₁ₐEᵢᵢE_{iib}E₄ | | E₄*E₅ | E_{f} |
| 1048 | E_{1b}EᵢᵢE_{iib}E₄ | | E₄*E₅ | E_{f} |
| 1049 | E₁ₐEᵢᵢᵢ | | E₄*E₅ | E_{f} |
| 1050 | E_{1b}Eᵢᵢᵢ | | E₄*E₅ | E_{f} |
| 1051 | E₁ₐEᵢᵢᵢE₄ | | E₄*E₅ | E_{f} |
| 1052 | E_{1b}EᵢᵢᵢE₄ | | E₄*E₅ | E_{f} |
| 1053 | E₁ₐEᵢᵥ | | E₄*E₅ | E_{f} |
| 1054 | E_{1b}Eᵢᵥ | | E₄*E₅ | E_{f} |
| 1055 | E₁ₐEᵢᵥE₄ | | E₄*E₅ | E_{f} |
| 1056 | E_{1b}EᵢᵥE₄ | | E₄*E₅ | E_{f} |
| 1057 | E₁ₐ | AlkL | | E_{f} |
| 1058 | E_{1b} | AlkL | | E_{f} |
| 1059 | E₁ₐEᵢ | AlkL | | E_{f} |
| 1060 | E_{1b}Eᵢ | AlkL | | E_{f} |
| 1061 | E₁ₐEᵢᵢ | AlkL | | E_{f} |
| 1062 | E_{1b}Eᵢᵢ | AlkL | | E_{f} |
| 1063 | E₁ₐEᵢᵢE_{iib} | AlkL | | E_{f} |
| 1064 | E_{1b}EᵢᵢE_{iib} | AlkL | | E_{f} |
| 1065 | E₁ₐE₄ | AlkL | | E_{f} |
| 1066 | E_{1b}E₄ | AlkL | | E_{f} |
| 1067 | E₁ₐEᵢE₄ | AlkL | | E_{f} |
| 1068 | E_{1b}EᵢE₄ | AlkL | | E_{f} |
| 1069 | E₁ₐEᵢᵢE₄ | AlkL | | E_{f} |
| 1070 | E_{1b}EᵢᵢE₄ | AlkL | | E_{f} |
| 1071 | E₁ₐEᵢᵢE_{iib}E₄ | AlkL | | E_{f} |
| 1072 | E_{1b}EᵢᵢE_{iib}E₄ | AlkL | | E_{f} |
| 1073 | E₁ₐEᵢᵢᵢ | AlkL | | E_{f} |
| 1074 | E_{1b}Eᵢᵢᵢ | AlkL | | E_{f} |
| 1075 | E₁ₐEᵢᵢᵢE₄ | AlkL | | E_{f} |
| 1076 | E_{1b}EᵢᵢᵢE₄ | AlkL | | E_{f} |
| 1077 | E₁ₐEᵢᵥ | AlkL | | E_{f} |
| 1078 | E_{1b}Eᵢᵥ | AlkL | | E_{f} |
| 1079 | E₁ₐEᵢᵥE₄ | AlkL | | E_{f} |
| 1080 | E_{1b}EᵢᵥE₄ | AlkL | | E_{f} |
| 1081 | E₁ₐ | AlkL | E₄* | E_{f} |
| 1082 | E_{1b} | AlkL | E₄* | E_{f} |
| 1083 | E₁ₐEᵢ | AlkL | E₄* | E_{f} |
| 1084 | E_{1b}Eᵢ | AlkL | E₄* | E_{f} |
| 1085 | E₁ₐEᵢᵢ | AlkL | E₄* | E_{f} |
| 1086 | E_{1b}Eᵢᵢ | AlkL | E₄* | E_{f} |
| 1087 | E₁ₐEᵢᵢE_{iib} | AlkL | E₄* | E_{f} |
| 1088 | E_{1b}EᵢᵢE_{iib} | AlkL | E₄* | E_{f} |
| 1089 | E₁ₐE₄ | AlkL | E₄* | E_{f} |
| 1090 | E_{1b}E₄ | AlkL | E₄* | E_{f} |
| 1091 | E₁ₐEᵢE₄ | AlkL | E₄* | E_{f} |
| 1092 | E_{1b}EᵢE₄ | AlkL | E₄* | E_{f} |
| 1093 | E₁ₐEᵢᵢE₄ | AlkL | E₄* | E_{f} |
| 1094 | E_{1b}EᵢᵢE₄ | AlkL | E₄* | E_{f} |
| 1095 | E₁ₐEᵢᵢE_{iib}E₄ | AlkL | E₄* | E_{f} |
| 1096 | E_{1b}EᵢᵢE_{iib}E₄ | AlkL | E₄* | E_{f} |
| 1097 | E₁ₐEᵢᵢᵢ | AlkL | E₄* | E_{f} |
| 1098 | E_{1b}Eᵢᵢᵢ | AlkL | E₄* | E_{f} |
| 1099 | E₁ₐEᵢᵢᵢE₄ | AlkL | E₄* | E_{f} |
| 1100 | E_{1b}EᵢᵢᵢE₄ | AlkL | E₄* | E_{f} |
| 1101 | E₁ₐEᵢᵥ | AlkL | E₄* | E_{f} |
| 1102 | E_{1b}Eᵢᵥ | AlkL | E₄* | E_{f} |
| 1103 | E₁ₐEᵢᵥE₄ | AlkL | E₄* | E_{f} |
| 1104 | E_{1b}EᵢᵥE₄ | AlkL | E₄* | E_{f} |
| 1105 | E₁ₐ | AlkL | E₅ | E_{f} |
| 1106 | E_{1b} | AlkL | E₅ | E_{f} |
| 1107 | E₁ₐEᵢ | AlkL | E₅ | E_{f} |
| 1108 | E_{1b}Eᵢ | AlkL | E₅ | E_{f} |
| 1109 | E₁ₐEᵢᵢ | AlkL | E₅ | E_{f} |
| 1110 | E_{1b}Eᵢᵢ | AlkL | E₅ | E_{f} |
| 1111 | E₁ₐEᵢᵢE_{iib} | AlkL | E₅ | E_{f} |
| 1112 | E_{1b}EᵢᵢE_{iib} | AlkL | E₅ | E_{f} |
| 1113 | E₁ₐE₄ | AlkL | E₅ | E_{f} |
| 1114 | E_{1b}E₄ | AlkL | E₅ | E_{f} |
| 1115 | E₁ₐEᵢE₄ | AlkL | E₅ | E_{f} |
| 1116 | E_{1b}EᵢE₄ | AlkL | E₅ | E_{f} |
| 1117 | E₁ₐEᵢᵢE₄ | AlkL | E₅ | E_{f} |
| 1118 | E_{1b}EᵢᵢE₄ | AlkL | E₅ | E_{f} |
| 1119 | E₁ₐEᵢᵢE_{iib}E₄ | AlkL | E₅ | E_{f} |
| 1120 | E_{1b}EᵢᵢE_{iib}E₄ | AlkL | E₅ | E_{f} |
| 1121 | E₁ₐEᵢᵢᵢ | AlkL | E₅ | E_{f} |
| 1122 | E_{1b}Eᵢᵢᵢ | AlkL | E₅ | E_{f} |
| 1123 | E₁ₐEᵢᵢᵢE₄ | AlkL | E₅ | E_{f} |
| 1124 | E_{1b}EᵢᵢᵢE₄ | AlkL | E₅ | E_{f} |
| 1125 | E₁ₐEᵢᵥ | AlkL | E₅ | E_{f} |
| 1126 | E_{1b}Eᵢᵥ | AlkL | E₅ | E_{f} |
| 1127 | E₁ₐEᵢᵥE₄ | AlkL | E₅ | E_{f} |
| 1128 | E_{1b}EᵢᵥE₄ | AlkL | E₅ | E_{f} |
| 1129 | E₁ₐ | AlkL | E₄*E₅ | E_{f} |
| 1130 | E_{1b} | AlkL | E₄*E₅ | E_{f} |
| 1131 | E₁ₐEᵢ | AlkL | E₄*E₅ | E_{f} |
| 1132 | E_{1b}Eᵢ | AlkL | E₄*E₅ | E_{f} |
| 1133 | E₁ₐEᵢᵢ | AlkL | E₄*E₅ | E_{f} |
| 1134 | E_{1b}Eᵢᵢ | AlkL | E₄*E₅ | E_{f} |
| 1135 | E₁ₐEᵢᵢE_{iib} | AlkL | E₄*E₅ | E_{f} |
| 1136 | E_{1b}EᵢᵢE_{iib} | AlkL | E₄*E₅ | E_{f} |
| 1137 | E₁ₐE₄ | AlkL | E₄*E₅ | E_{f} |
| 1138 | E_{1b}E₄ | AlkL | E₄*E₅ | E_{f} |
| 1139 | E₁ₐEᵢE₄ | AlkL | E₄*E₅ | E_{f} |
| 1140 | E_{1b}EᵢE₄ | AlkL | E₄*E₅ | E_{f} |
| 1141 | E₁ₐEᵢᵢE₄ | AlkL | E₄*E₅ | E_{f} |
| 1142 | E_{1b}EᵢᵢE₄ | AlkL | E₄*E₅ | E_{f} |
| 1143 | E₁ₐEᵢᵢE_{iib}E₄ | AlkL | E₄*E₅ | E_{f} |
| 1144 | E_{1b}EᵢᵢE_{iib}E₄ | AlkL | E₄*E₅ | E_{f} |
| 1145 | E₁ₐEᵢᵢᵢ | AlkL | E₄*E₅ | E_{f} |
| 1146 | E_{1b}Eᵢᵢᵢ | AlkL | E₄*E₅ | E_{f} |
| 1147 | E₁ₐEᵢᵢᵢE₄ | AlkL | E₄*E₅ | E_{f} |
| 1148 | E_{1b}EᵢᵢᵢE₄ | AlkL | E₄*E₅ | E_{f} |
| 1149 | E₁ₐEᵢᵥ | AlkL | E₄*E₅ | E_{f} |
| 1150 | E_{1b}Eᵢᵥ | AlkL | E₄*E₅ | E_{f} |
| 1151 | E₁ₐEᵢᵥE₄ | AlkL | E₄*E₅ | E_{f} |
| 1152 | E_{1b}EᵢᵥE₄ | AlkL | E₄*E₅ | E_{f} |

Microorganisms quite especially preferred according to the invention (abbreviated as MO) are outstandingly suitable for the production of ω-hydroxycarboxylate esters or ω-oxocarboxylate esters and have increased or decreased enzyme activities (abbreviated as E) described in the following table, where these can in addition advantageously be combined with an increased enzyme activity compared to the wild type of the microorganism, which is described for the 3-ketoacyl-ACP (Acyl Carrrier Protein) synthase III (EC 2.3.1.41), in particular that from plants, preferably that from plants the seeds whereof contain fatty acids with alkyl residues shorter than 14 C atoms, and particularly preferably that from plants of the genera Cuphea, Elaeis, Cocos, Umbellularia and Cinnamomum and gene products selected from AccA, AccB, AccC, AccD, AceE, AceF, Lpd, AcpP, FabA, FabB, FabD, FabF, FabG, FabH, FabI, FabZ, PanD, PanK, UdhA, PntA or PntB.

Any combinations of at least two of these enzyme activities can advantageously be increased.

In addition, moreover, it can be advantageous if the microorganism is provided with a lower enzyme activity compared to the wild type of the microorganism, which is described for the gene products selected from TdcE, PflA, PflB, PflC, PflD, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, singly or in any combination.

| | | |
|---|---|---|
| MO | E increased | E decreased |

| | | | | |
|---|---|---|---|---|
| 1 | E₁ₐ | EᵥEᵥᵢ | | |
| 2 | E_{1b} | EᵥEᵥᵢ | | |
| 3 | E₁ₐEᵢ | EᵥEᵥᵢ | | |
| 4 | E_{1b}Eᵢ | EᵥEᵥᵢ | | |
| 5 | E₁ₐEᵢᵢ | EᵥEᵥᵢ | | |
| 6 | E_{1b}Eᵢᵢ | EᵥEᵥᵢ | | |
| 7 | E₁ₐEᵢᵢE_{iib} | EᵥEᵥᵢ | | |
| 8 | E_{1b}EᵢᵢE_{iib} | EᵥEᵥᵢ | | |
| 9 | E₁ₐE₄ | EᵥEᵥᵢ | | |
| 10 | E_{1b}E₄ | EᵥEᵥᵢ | | |
| 11 | E₁ₐEᵢE₄ | EᵥEᵥᵢ | | |
| 12 | E_{1b}EᵢE₄ | EᵥEᵥᵢ | | |
| 13 | E₁ₐEᵢᵢE₄ | EᵥEᵥᵢ | | |
| 14 | E_{1b}EᵢᵢE₄ | EᵥEᵥᵢ | | |
| 15 | E₁ₐEᵢᵢE_{iib}E₄ | EᵥEᵥᵢ | | |
| 16 | E_{1b}EᵢᵢE_{iib}E₄ | EᵥEᵥᵢ | | |
| 17 | E₁ₐEᵢᵢᵢ | EᵥEᵥᵢ | | |
| 18 | E_{1b}Eᵢᵢᵢ | EᵥEᵥᵢ | | |
| 19 | E₁ₐEᵢᵢᵢE₄ | EᵥEᵥᵢ | | |
| 20 | E_{1b}EᵢᵢᵢE₄ | EᵥEᵥᵢ | | |
| 21 | E₁ₐEᵢᵥ | EᵥEᵥᵢ | | |
| 22 | E_{1b}Eᵢᵥ | EᵥEᵥᵢ | | |
| 23 | E₁ₐEᵢᵥE₄ | EᵥEᵥᵢ | | |
| 24 | E_{1b}EᵢᵥE₄ | EᵥEᵥᵢ | | |
| 25 | E₁ₐ | EᵥEᵥᵢ | E₄* | |
| 26 | E_{1b} | EᵥEᵥᵢ | E₄* | |
| 27 | E₁ₐEᵢ | EᵥEᵥᵢ | E₄* | |
| 28 | E_{1b}Eᵢ | EᵥEᵥᵢ | E₄* | |
| 29 | E₁ₐEᵢᵢ | EᵥEᵥᵢ | E₄* | |
| 30 | E_{1b}Eᵢᵢ | EᵥEᵥᵢ | E₄* | |
| 31 | E₁ₐEᵢᵢE_{iib} | EᵥEᵥᵢ | E₄* | |
| 32 | E_{1b}EᵢᵢE_{iib} | EᵥEᵥᵢ | E₄* | |
| 33 | E₁ₐE₄ | EᵥEᵥᵢ | E₄* | |
| 34 | E_{1b}E₄ | EᵥEᵥᵢ | E₄* | |
| 35 | E₁ₐEᵢE₄ | EᵥEᵥᵢ | E₄* | |
| 36 | E_{1b}EᵢE₄ | EᵥEᵥᵢ | E₄* | |
| 37 | E₁ₐEᵢᵢE₄ | EᵥEᵥᵢ | E₄* | |
| 38 | E_{1b}EᵢᵢE₄ | EᵥEᵥᵢ | E₄* | |
| 39 | E₁ₐEᵢᵢE_{iib}E₄ | EᵥEᵥᵢ | E₄* | |
| 40 | E_{1b}EᵢᵢE_{iib}E₄ | EᵥEᵥᵢ | E₄* | |
| 41 | E₁ₐEᵢᵢᵢ | EᵥEᵥᵢ | E₄* | |
| 42 | E_{1b}Eᵢᵢᵢ | EᵥEᵥᵢ | E₄* | |
| 43 | E₁ₐEᵢᵢᵢE₄ | EᵥEᵥᵢ | E₄* | |
| 44 | E_{1b}EᵢᵢᵢE₄ | EᵥEᵥᵢ | E₄* | |
| 45 | E₁ₐEᵢᵥ | EᵥEᵥᵢ | E₄* | |
| 46 | E_{1b}Eᵢᵥ | EᵥEᵥᵢ | E₄* | |
| 47 | E₁ₐEᵢᵥE₄ | EᵥEᵥᵢ | E₄* | |
| 48 | E_{1b}EᵢᵥE₄ | EᵥEᵥᵢ | E₄* | |
| 49 | E₁ₐ | EᵥEᵥᵢ | E₅ | |
| 50 | E_{1b} | EᵥEᵥᵢ | E₅ | |
| 51 | E₁ₐEᵢ | EᵥEᵥᵢ | E₅ | |
| 52 | E_{1b}Eᵢ | EᵥEᵥᵢ | E₅ | |
| 53 | E₁ₐEᵢᵢ | EᵥEᵥᵢ | E₅ | |
| 54 | E_{1b}Eᵢᵢ | EᵥEᵥᵢ | E₅ | |
| 55 | E₁ₐEᵢᵢE_{iib} | EᵥEᵥᵢ | E₅ | |
| 56 | E_{1b}EᵢᵢE_{iib} | EᵥEᵥᵢ | E₅ | |
| 57 | E₁ₐE₄ | EᵥEᵥᵢ | E₅ | |
| 58 | E_{1b}E₄ | EᵥEᵥᵢ | E₅ | |
| 59 | E₁ₐEᵢE₄ | EᵥEᵥᵢ | E₅ | |
| 60 | E_{1b}EᵢE₄ | EᵥEᵥᵢ | E₅ | |
| 61 | E₁ₐEᵢᵢE₄ | EᵥEᵥᵢ | E₅ | |
| 62 | E_{1b}EᵢᵢE₄ | EᵥEᵥᵢ | E₅ | |
| 63 | E₁ₐEᵢᵢE_{iib}E₄ | EᵥEᵥᵢ | E₅ | |
| 64 | E_{1b}EᵢᵢE_{iib}E₄ | EᵥEᵥᵢ | E₅ | |
| 65 | E₁ₐEᵢᵢᵢ | EᵥEᵥᵢ | E₅ | |
| 66 | E_{1b}Eᵢᵢᵢ | EᵥEᵥᵢ | E₅ | |
| 67 | E₁ₐEᵢᵢᵢE₄ | EᵥEᵥᵢ | E₅ | |
| 68 | E_{1b}EᵢᵢᵢE₄ | EᵥEᵥᵢ | E₅ | |
| 69 | E₁ₐEᵢᵥ | EᵥEᵥᵢ | E₅ | |
| 70 | E_{1b}Eᵢᵥ | EᵥEᵥᵢ | E₅ | |
| 71 | E₁ₐEᵢᵥE₄ | EᵥEᵥᵢ | E₅ | |
| 72 | E_{1b}EᵢᵥE₄ | EᵥEᵥᵢ | E₅ | |
| 73 | E₁ₐ | EᵥEᵥᵢ | E₄*E₅ | |
| 74 | E_{1b} | EᵥEᵥᵢ | E₄*E₅ | |
| 75 | E₁ₐEᵢ | EᵥEᵥᵢ | E₄*E₅ | |
| 76 | E_{1b}Eᵢ | EᵥEᵥᵢ | E₄*E₅ | |
| 77 | E₁ₐEᵢᵢ | EᵥEᵥᵢ | E₄*E₅ | |
| 78 | E_{1b}Eᵢᵢ | EᵥEᵥᵢ | E₄*E₅ | |
| 79 | E₁ₐEᵢᵢE_{iib} | EᵥEᵥᵢ | E₄*E₅ | |
| 80 | E_{1b}EᵢᵢE_{iib} | EᵥEᵥᵢ | E₄*E₅ | |
| 81 | E₁ₐE₄ | EᵥEᵥᵢ | E₄*E₅ | |
| 82 | E_{1b}E₄ | EᵥEᵥᵢ | E₄*E₅ | |
| 83 | E₁ₐEᵢE₄ | EᵥEᵥᵢ | E₄*E₅ | |
| 84 | E_{1b}EᵢE₄ | EᵥEᵥᵢ | E₄*E₅ | |
| 85 | E₁ₐEᵢᵢE₄ | EᵥEᵥᵢ | E₄*E₅ | |
| 86 | E_{1b}EᵢᵢE₄ | EᵥEᵥᵢ | E₄*E₅ | |
| 87 | E₁ₐEᵢᵢE_{iib}E₄ | EᵥEᵥᵢ | E₄*E₅ | |
| 88 | E_{1b}EᵢᵢE_{iib}E₄ | EᵥEᵥᵢ | E₄*E₅ | |
| 89 | E₁ₐEᵢᵢᵢ | EᵥEᵥᵢ | E₄*E₅ | |
| 90 | E_{1b}Eᵢᵢᵢ | EᵥEᵥᵢ | E₄*E₅ | |
| 91 | E₁ₐEᵢᵢᵢE₄ | EᵥEᵥᵢ | E₄*E₅ | |
| 92 | E_{1b}EᵢᵢᵢE₄ | EᵥEᵥᵢ | E₄*E₅ | |
| 93 | E₁ₐEᵢᵥ | EᵥEᵥᵢ | E₄*E₅ | |
| 94 | E_{1b}Eᵢᵥ | EᵥEᵥᵢ | E₄*E₅ | |
| 95 | E₁ₐEᵢᵥE₄ | EᵥEᵥᵢ | E₄*E₅ | |
| 96 | E_{1b}EᵢᵥE₄ | EᵥEᵥᵢ | E₄*E₅ | |
| 97 | E₁ₐ | AlkLEᵥEᵥᵢ | | |
| 98 | E_{1b} | AlkLEᵥEᵥᵢ | | |
| 99 | E₁ₐEᵢ | AlkLEᵥEᵥᵢ | | |
| 100 | E_{1b}Eᵢ | AlkLEᵥEᵥᵢ | | |
| 101 | E₁ₐEᵢᵢ | AlkLEᵥEᵥᵢ | | |
| 102 | E_{1b}Eᵢᵢ | AlkLEᵥEᵥᵢ | | |
| 103 | E₁ₐEᵢᵢE_{iib} | AlkLEᵥEᵥᵢ | | |
| 104 | E_{1b}EᵢᵢE_{iib} | AlkLEᵥEᵥᵢ | | |
| 105 | E₁ₐE₄ | AlkLEᵥEᵥᵢ | | |
| 106 | E_{1b}E₄ | AlkLEᵥEᵥᵢ | | |
| 107 | E₁ₐEᵢE₄ | AlkLEᵥEᵥᵢ | | |
| 108 | E_{1b}EᵢE₄ | AlkLEᵥEᵥᵢ | | |
| 109 | E₁ₐEᵢᵢE₄ | AlkLEᵥEᵥᵢ | | |
| 110 | E_{1b}EᵢᵢE₄ | AlkLEᵥEᵥᵢ | | |
| 111 | E₁ₐEᵢᵢE_{iib}E₄ | AlkLEᵥEᵥᵢ | | |
| 112 | E_{1b}EᵢᵢE_{iib}E₄ | AlkLEᵥEᵥᵢ | | |
| 113 | E₁ₐEᵢᵢᵢ | AlkLEᵥEᵥᵢ | | |
| 114 | E_{1b}Eᵢᵢᵢ | AlkLEᵥEᵥᵢ | | |
| 115 | E₁ₐEᵢᵢᵢE₄ | AlkLEᵥEᵥᵢ | | |
| 116 | E_{1b}EᵢᵢᵢE₄ | AlkLEᵥEᵥᵢ | | |
| 117 | E₁ₐEᵢᵥ | AlkLEᵥEᵥᵢ | | |
| 118 | E_{1b}Eᵢᵥ | AlkLEᵥEᵥᵢ | | |
| 119 | E₁ₐEᵢᵥE₄ | AlkLEᵥEᵥᵢ | | |
| 120 | E_{1b}EᵢᵥE₄ | AlkLEᵥEᵥᵢ | | |
| 121 | E₁ₐ | AlkLEᵥEᵥᵢ | E₄* | |
| 122 | E_{1b} | AlkLEᵥEᵥᵢ | E₄* | |
| 123 | E₁ₐEᵢ | AlkLEᵥEᵥᵢ | E₄* | |
| 124 | E_{1b}Eᵢ | AlkLEᵥEᵥᵢ | E₄* | |
| 125 | E₁ₐEᵢᵢ | AlkLEᵥEᵥᵢ | E₄* | |
| 126 | E_{1b}Eᵢᵢ | AlkLEᵥEᵥᵢ | E₄* | |
| 127 | E₁ₐEᵢᵢE_{iib} | AlkLEᵥEᵥᵢ | E₄* | |
| 128 | E_{1b}EᵢᵢE_{iib} | AlkLEᵥEᵥᵢ | E₄* | |
| 129 | E₁ₐE₄ | AlkLEᵥEᵥᵢ | E₄* | |
| 130 | E_{1b}E₄ | AlkLEᵥEᵥᵢ | E₄* | |
| 131 | E₁ₐEᵢE₄ | AlkLEᵥEᵥᵢ | E₄* | |
| 132 | E_{1b}EᵢE₄ | AlkLEᵥEᵥᵢ | E₄* | |
| 133 | E₁ₐEᵢᵢE₄ | AlkLEᵥEᵥᵢ | E₄* | |
| 134 | E_{1b}EᵢᵢE₄ | AlkLEᵥEᵥᵢ | E₄* | |
| 135 | E₁ₐEᵢᵢE_{iib}E₄ | AlkLEᵥEᵥᵢ | E₄* | |
| 136 | E_{1b}EᵢᵢE_{iib}E₄ | AlkLEᵥEᵥᵢ | E₄* | |
| 137 | E₁ₐEᵢᵢᵢ | AlkLEᵥEᵥᵢ | E₄* | |
| 138 | E_{1b}Eᵢᵢᵢ | AlkLEᵥEᵥᵢ | E₄* | |
| 139 | E₁ₐEᵢᵢᵢE₄ | AlkLEᵥEᵥᵢ | E₄* | |
| 140 | E_{1b}EᵢᵢᵢE₄ | AlkLEᵥEᵥᵢ | E₄* | |
| 141 | E₁ₐEᵢᵥ | AlkLEᵥEᵥᵢ | E₄* | |
| 142 | E_{1b}Eᵢᵥ | AlkLEᵥEᵥᵢ | E₄* | |
| 143 | E₁ₐEᵢᵥE₄ | AlkLEᵥEᵥᵢ | E₄* | |
| 144 | E_{1b}EᵢᵥE₄ | AlkLEᵥEᵥᵢ | E₄* | |
| 145 | E₁ₐ | AlkLEᵥEᵥᵢ | E₅ | |
| 146 | E_{1b} | AlkLEᵥEᵥᵢ | E₅ | |
| 147 | E₁ₐEᵢ | AlkLEᵥEᵥᵢ | E₅ | |
| 148 | E_{1b}Eᵢ | AlkLEᵥEᵥᵢ | E₅ | |
| 149 | E₁ₐEᵢᵢ | AlkLEᵥEᵥᵢ | E₅ | |
| 150 | E_{1b}Eᵢᵢ | AlkLEᵥEᵥᵢ | E₅ | |
| 151 | E₁ₐEᵢᵢE_{iib} | AlkLEᵥEᵥᵢ | E₅ | |
| 152 | E_{1b}EᵢᵢE_{iib} | AlkLEᵥEᵥᵢ | E₅ | |
| 153 | E₁ₐE₄ | AlkLEᵥEᵥᵢ | E₅ | |
| 154 | E_{1b}E₄ | AlkLEᵥEᵥᵢ | E₅ | |
| 155 | E₁ₐEᵢE₄ | AlkLEᵥEᵥᵢ | E₅ | |
| 156 | E_{1b}EᵢE₄ | AlkLEᵥEᵥᵢ | E₅ | |
| 157 | E₁ₐEᵢᵢE₄ | AlkLEᵥEᵥᵢ | E₅ | |
| 158 | E_{1b}EᵢᵢE₄ | AlkLEᵥEᵥᵢ | E₅ | |
| 159 | E₁ₐEᵢᵢE_{iib}E₄ | AlkLEᵥEᵥᵢ | E₅ | |
| 160 | E_{1b}EᵢᵢE_{iib}E₄ | AlkLEᵥEᵥᵢ | E₅ | |
| 161 | E₁ₐEᵢᵢᵢ | AlkLEᵥEᵥᵢ | E₅ | |
| 162 | E_{1b}Eᵢᵢᵢ | AlkLEᵥEᵥᵢ | E₅ | |
| 163 | E₁ₐEᵢᵢᵢE₄ | AlkLEᵥEᵥᵢ | E₅ | |
| 164 | E_{1b}EᵢᵢᵢE₄ | AlkLEᵥEᵥᵢ | E₅ | |
| 165 | E₁ₐEᵢᵥ | AlkLEᵥEᵥᵢ | E₅ | |
| 166 | E_{1b}Eᵢᵥ | AlkLEᵥEᵥᵢ | E₅ | |
| 167 | E₁ₐEᵢᵥE₄ | AlkLEᵥEᵥᵢ | E₅ | |
| 168 | E_{1b}EᵢᵥE₄ | AlkLEᵥEᵥᵢ | E₅ | |
| 169 | E₁ₐ | AlkLEᵥEᵥᵢ | E₄*E₅ | |
| 170 | E_{1b} | AlkLEᵥEᵥᵢ | E₄*E₅ | |
| 171 | E₁ₐEᵢ | AlkLEᵥEᵥᵢ | E₄*E₅ | |
| 172 | E_{1b}Eᵢ | AlkLEᵥEᵥᵢ | E₄*E₅ | |
| 173 | E₁ₐEᵢᵢ | AlkLEᵥEᵥᵢ | E₄*E₅ | |
| 174 | E_{1b}Eᵢᵢ | AlkLEᵥEᵥᵢ | E₄*E₅ | |
| 175 | E₁ₐEᵢᵢE_{iib} | AlkLEᵥEᵥᵢ | E₄*E₅ | |
| 176 | E_{1b}EᵢᵢE_{iib} | AlkLEᵥEᵥᵢ | E₄*E₅ | |
| 177 | E₁ₐE₄ | AlkLEᵥEᵥᵢ | E₄*E₅ | |
| 178 | E_{1b}E₄ | AlkLEᵥEᵥᵢ | E₄*E₅ | |
| 179 | E₁ₐEᵢE₄ | AlkLEᵥEᵥᵢ | E₄*E₅ | |
| 180 | E_{1b}EᵢE₄ | AlkLEᵥEᵥᵢ | E₄*E₅ | |
| 181 | E₁ₐEᵢᵢE₄ | AlkLEᵥEᵥᵢ | E₄*E₅ | |
| 182 | E_{1b}EᵢᵢE₄ | AlkLEᵥEᵥᵢ | E₄*E₅ | |
| 183 | E₁ₐEᵢᵢE_{iib}E₄ | AlkLEᵥEᵥᵢ | E₄*E₅ | |
| 184 | E_{1b}EᵢᵢE_{iib}E₄ | AlkLEᵥEᵥᵢ | E₄*E₅ | |
| 185 | E₁ₐEᵢᵢᵢ | AlkLEᵥEᵥᵢ | E₄*E₅ | |
| 186 | E_{1b}Eᵢᵢᵢ | AlkLEᵥEᵥᵢ | E₄*E₅ | |
| 187 | E₁ₐEᵢᵢᵢE₄ | AlkLEᵥEᵥᵢ | E₄*E₅ | |
| 188 | E_{1b}EᵢᵢᵢE₄ | AlkLEᵥEᵥᵢ | E₄*E₅ | |
| 189 | E₁ₐEᵢᵥ | AlkLEᵥEᵥᵢ | E₄*E₅ | |
| 190 | E_{1b}Eᵢᵥ | AlkLEᵥEᵥᵢ | E₄*E₅ | |
| 191 | E₁ₐEᵢᵥE₄ | AlkLEᵥEᵥᵢ | E₄*E₅ | |
| 192 | E_{1b}EᵢᵥE₄ | AlkLEᵥEᵥᵢ | E₄*E₅ | |
| 193 | E₁ₐ | EᵥEᵥᵢ | | Eₐ |
| 194 | E_{1b} | EᵥEᵥᵢ | | Eₐ |
| 195 | E₁ₐEᵢ | EᵥEᵥᵢ | | Eₐ |
| 196 | E_{1b}Eᵢ | EᵥEᵥᵢ | | Eₐ |
| 197 | E₁ₐEᵢᵢ | EᵥEᵥᵢ | | Eₐ |
| 198 | E_{1b}Eᵢᵢ | EᵥEᵥᵢ | | Eₐ |
| 199 | E₁ₐEᵢᵢE_{iib} | EᵥEᵥᵢ | | Eₐ |
| 200 | E_{1b}EᵢᵢE_{iib} | EᵥEᵥᵢ | | Eₐ |
| 201 | E₁ₐE₄ | EᵥEᵥᵢ | | Eₐ |
| 202 | E_{1b}E₄ | EᵥEᵥᵢ | | Eₐ |
| 203 | E₁ₐEᵢE₄ | EᵥEᵥᵢ | | Eₐ |
| 204 | E_{1b}EᵢE₄ | EᵥEᵥᵢ | | Eₐ |
| 205 | E₁ₐEᵢᵢE₄ | EᵥEᵥᵢ | | Eₐ |
| 206 | E_{1b}EᵢᵢE₄ | EᵥEᵥᵢ | | Eₐ |
| 207 | E₁ₐEᵢᵢE_{iib}E₄ | EᵥEᵥᵢ | | Eₐ |
| 208 | E_{1b}EᵢᵢE_{iib}E₄ | EᵥEᵥᵢ | | Eₐ |
| 209 | E₁ₐEᵢᵢᵢ | EᵥEᵥᵢ | | Eₐ |
| 210 | E_{1b}Eᵢᵢᵢ | EᵥEᵥᵢ | | Eₐ |
| 211 | E₁ₐEᵢᵢᵢE₄ | EᵥEᵥᵢ | | Eₐ |
| 212 | E_{1b}EᵢᵢᵢE₄ | EᵥEᵥᵢ | | Eₐ |
| 213 | E₁ₐEᵢᵥ | EᵥEᵥᵢ | | Eₐ |
| 214 | E_{1b}Eᵢᵥ | EᵥEᵥᵢ | | Eₐ |
| 215 | E₁ₐEᵢᵥE₄ | EᵥEᵥᵢ | | Eₐ |
| 216 | E_{1b}EᵢᵥE₄ | EᵥEᵥᵢ | | Eₐ |
| 217 | E₁ₐ | EᵥEᵥᵢ | E₄* | Eₐ |
| 218 | E_{1b} | EᵥEᵥᵢ | E₄* | Eₐ |
| 219 | E₁ₐEᵢ | EᵥEᵥᵢ | E₄* | Eₐ |
| 220 | E_{1b}Eᵢ | EᵥEᵥᵢ | E₄* | Eₐ |
| 221 | E₁ₐEᵢᵢ | EᵥEᵥᵢ | E₄* | Eₐ |
| 222 | E_{1b}Eᵢᵢ | EᵥEᵥᵢ | E₄* | Eₐ |
| 223 | E₁ₐEᵢᵢE_{iib} | EᵥEᵥᵢ | E₄* | Eₐ |
| 224 | E_{1b}EᵢᵢE_{iib} | EᵥEᵥᵢ | E₄* | Eₐ |
| 225 | E₁ₐE₄ | EᵥEᵥᵢ | E₄* | Eₐ |
| 226 | E_{1b}E₄ | EᵥEᵥᵢ | E₄* | Eₐ |
| 227 | E₁ₐEᵢE₄ | EᵥEᵥᵢ | E₄* | Eₐ |
| 228 | E_{1b}EᵢE₄ | EᵥEᵥᵢ | E₄* | Eₐ |
| 229 | E₁ₐEᵢᵢE₄ | EᵥEᵥᵢ | E₄* | Eₐ |
| 230 | E_{1b}EᵢᵢE₄ | EᵥEᵥᵢ | E₄* | Eₐ |
| 231 | E₁ₐEᵢᵢE_{iib}E₄ | EᵥEᵥᵢ | E₄* | Eₐ |
| 232 | E_{1b}EᵢᵢE_{iib}E₄ | EᵥEᵥᵢ | E₄* | Eₐ |
| 233 | E₁ₐEᵢᵢᵢ | EᵥEᵥᵢ | E₄* | Eₐ |
| 234 | E_{1b}Eᵢᵢᵢ | EᵥEᵥᵢ | E₄* | Eₐ |
| 235 | E₁ₐEᵢᵢᵢE₄ | EᵥEᵥᵢ | E₄* | Eₐ |
| 236 | E_{1b}EᵢᵢᵢE₄ | EᵥEᵥᵢ | E₄* | Eₐ |
| 237 | E₁ₐEᵢᵥ | EᵥEᵥᵢ | E₄* | Eₐ |
| 238 | E_{1b}Eᵢᵥ | EᵥEᵥᵢ | E₄* | Eₐ |
| 239 | E₁ₐEᵢᵥE₄ | EᵥEᵥᵢ | E₄* | Eₐ |
| 240 | E_{1b}EᵢᵥE₄ | EᵥEᵥᵢ | E₄* | Eₐ |
| 241 | E₁ₐ | EᵥEᵥᵢ | E₅ | Eₐ |
| 242 | E_{1b} | EᵥEᵥᵢ | E₅ | Eₐ |
| 243 | E₁ₐEᵢ | EᵥEᵥᵢ | E₅ | Eₐ |
| 244 | E_{1b}Eᵢ | EᵥEᵥᵢ | E₅ | Eₐ |
| 245 | E₁ₐEᵢᵢ | EᵥEᵥᵢ | E₅ | Eₐ |
| 246 | E_{1b}Eᵢᵢ | EᵥEᵥᵢ | E₅ | Eₐ |
| 247 | E₁ₐEᵢᵢE_{iib} | EᵥEᵥᵢ | E₅ | Eₐ |
| 248 | E_{1b}EᵢᵢE_{iib} | EᵥEᵥᵢ | E₅ | Eₐ |
| 249 | E₁ₐE₄ | EᵥEᵥᵢ | E₅ | Eₐ |
| 250 | E_{1b}E₄ | EᵥEᵥᵢ | E₅ | Eₐ |
| 251 | E₁ₐEᵢE₄ | EᵥEᵥᵢ | E₅ | Eₐ |
| 252 | E_{1b}EᵢE₄ | EᵥEᵥᵢ | E₅ | Eₐ |
| 253 | E₁ₐEᵢᵢE₄ | EᵥEᵥᵢ | E₅ | Eₐ |
| 254 | E_{1b}EᵢᵢE₄ | EᵥEᵥᵢ | E₅ | Eₐ |
| 255 | E₁ₐEᵢᵢE_{iib}E₄ | EᵥEᵥᵢ | E₅ | Eₐ |
| 256 | E_{1b}EᵢᵢE_{iib}E₄ | EᵥEᵥᵢ | E₅ | Eₐ |
| 257 | E₁ₐEᵢᵢᵢ | EᵥEᵥᵢ | E₅ | Eₐ |
| 258 | E_{1b}Eᵢᵢᵢ | EᵥEᵥᵢ | E₅ | Eₐ |
| 259 | E₁ₐEᵢᵢᵢE₄ | EᵥEᵥᵢ | E₅ | Eₐ |
| 260 | E_{1b}EᵢᵢᵢE₄ | EᵥEᵥᵢ | E₅ | Eₐ |
| 261 | E₁ₐEᵢᵥ | EᵥEᵥᵢ | E₅ | Eₐ |
| 262 | E_{1b}Eᵢᵥ | EᵥEᵥᵢ | E₅ | Eₐ |
| 263 | E₁ₐEᵢᵥE₄ | EᵥEᵥᵢ | E₅ | Eₐ |
| 264 | E_{1b}EᵢᵥE₄ | EᵥEᵥᵢ | E₅ | Eₐ |
| 265 | E₁ₐ | EᵥEᵥᵢ | E₄*E₅ | Eₐ |
| 266 | E_{1b} | EᵥEᵥᵢ | E₄*E₅ | Eₐ |
| 267 | E₁ₐEᵢ | EᵥEᵥᵢ | E₄*E₅ | Eₐ |
| 268 | E_{1b}Eᵢ | EᵥEᵥᵢ | E₄*E₅ | Eₐ |
| 269 | E₁ₐEᵢᵢ | EᵥEᵥᵢ | E₄*E₅ | Eₐ |
| 270 | E_{1b}Eᵢᵢ | EᵥEᵥᵢ | E₄*E₅ | Eₐ |
| 271 | E₁ₐEᵢᵢE_{iib} | EᵥEᵥᵢ | E₄*E₅ | Eₐ |
| 272 | E_{1b}EᵢᵢE_{iib} | EᵥEᵥᵢ | E₄*E₅ | Eₐ |
| 273 | E₁ₐE₄ | EᵥEᵥᵢ | E₄*E₅ | Eₐ |
| 274 | E_{1b}E₄ | EᵥEᵥᵢ | E₄*E₅ | Eₐ |
| 275 | E₁ₐEᵢE₄ | EᵥEᵥᵢ | E₄*E₅ | Eₐ |
| 276 | E_{1b}EᵢE₄ | EᵥEᵥᵢ | E₄*E₅ | Eₐ |
| 277 | E₁ₐEᵢᵢE₄ | EᵥEᵥᵢ | E₄*E₅ | Eₐ |
| 278 | E_{1b}EᵢᵢE₄ | EᵥEᵥᵢ | E₄*E₅ | Eₐ |
| 279 | E₁ₐEᵢᵢE_{iib}E₄ | EᵥEᵥᵢ | E₄*E₅ | Eₐ |
| 280 | E_{1b}EᵢᵢE_{iib}E₄ | EᵥEᵥᵢ | E₄*E₅ | Eₐ |
| 281 | E₁ₐEᵢᵢᵢ | EᵥEᵥᵢ | E₄*E₅ | Eₐ |
| 282 | E_{1b}Eᵢᵢᵢ | EᵥEᵥᵢ | E₄*E₅ | Eₐ |
| 283 | E₁ₐEᵢᵢᵢE₄ | EᵥEᵥᵢ | E₄*E₅ | Eₐ |
| 284 | E_{1b}EᵢᵢᵢE₄ | EᵥEᵥᵢ | E₄*E₅ | Eₐ |
| 285 | E₁ₐEᵢᵥ | EᵥEᵥᵢ | E₄*E₅ | Eₐ |
| 286 | E_{1b}Eᵢᵥ | EᵥEᵥᵢ | E₄*E₅ | Eₐ |
| 287 | E₁ₐEᵢᵥE₄ | EᵥEᵥᵢ | E₄*E₅ | Eₐ |
| 288 | E_{1b}EᵢᵥE₄ | EᵥEᵥᵢ | E₄*E₅ | Eₐ |
| 289 | E₁ₐ | AlkLEᵥEᵥᵢ | | Eₐ |
| 290 | E_{1b} | AlkLEᵥEᵥᵢ | | Eₐ |
| 291 | E₁ₐEᵢ | AlkLEᵥEᵥᵢ | | Eₐ |
| 292 | E_{1b}Eᵢ | AlkLEᵥEᵥᵢ | | Eₐ |
| 293 | E₁ₐEᵢᵢ | AlkLEᵥEᵥᵢ | | Eₐ |
| 294 | E_{1b}Eᵢᵢ | AlkLEᵥEᵥᵢ | | Eₐ |
| 295 | E₁ₐEᵢᵢE_{iib} | AlkLEᵥEᵥᵢ | | Eₐ |
| 296 | E_{1b}EᵢᵢE_{iib} | AlkLEᵥEᵥᵢ | | Eₐ |
| 297 | E₁ₐE₄ | AlkLEᵥEᵥᵢ | | Eₐ |
| 298 | E_{1b}E₄ | AlkLEᵥEᵥᵢ | | Eₐ |
| 299 | E₁ₐEᵢE₄ | AlkLEᵥEᵥᵢ | | Eₐ |
| 300 | E_{1b}EᵢE₄ | AlkLEᵥEᵥᵢ | | Eₐ |
| 301 | E₁ₐEᵢᵢE₄ | AlkLEᵥEᵥᵢ | | Eₐ |
| 302 | E_{1b}EᵢᵢE₄ | AlkLEᵥEᵥᵢ | | Eₐ |
| 303 | E₁ₐEᵢᵢE_{iib}E₄ | AlkLEᵥEᵥᵢ | | Eₐ |
| 304 | E_{1b}EᵢᵢE_{iib}E₄ | AlkLEᵥEᵥᵢ | | Eₐ |
| 305 | E₁ₐEᵢᵢᵢ | AlkLEᵥEᵥᵢ | | Eₐ |
| 306 | E_{1b}Eᵢᵢᵢ | AlkLEᵥEᵥᵢ | | Eₐ |
| 307 | E₁ₐEᵢᵢᵢE₄ | AlkLEᵥEᵥᵢ | | Eₐ |
| 308 | E_{1b}EᵢᵢᵢE₄ | AlkLEᵥEᵥᵢ | | Eₐ |
| 309 | E₁ₐEᵢᵥ | AlkLEᵥEᵥᵢ | | Eₐ |
| 310 | E_{1b}Eᵢᵥ | AlkLEᵥEᵥᵢ | | Eₐ |
| 311 | E₁ₐEᵢᵥE₄ | AlkLEᵥEᵥᵢ | | Eₐ |
| 312 | E_{1b}EᵢᵥE₄ | AlkLEᵥEᵥᵢ | | Eₐ |
| 313 | E₁ₐ | AlkLEᵥEᵥᵢ | E₄* | Eₐ |
| 314 | E_{1b} | AlkLEᵥEᵥᵢ | E₄* | Eₐ |
| 315 | E₁ₐEᵢ | AlkLEᵥEᵥᵢ | E₄* | Eₐ |
| 316 | E_{1b}Eᵢ | AlkLEᵥEᵥᵢ | E₄* | Eₐ |
| 317 | E₁ₐEᵢᵢ | AlkLEᵥEᵥᵢ | E₄* | Eₐ |
| 318 | E_{1b}Eᵢᵢ | AlkLEᵥEᵥᵢ | E₄* | Eₐ |
| 319 | E₁ₐEᵢᵢE_{iib} | AlkLEᵥEᵥᵢ | E₄* | Eₐ |
| 320 | E_{1b}EᵢᵢE_{iib} | AlkLEᵥEᵥᵢ | E₄* | Eₐ |
| 321 | E₁ₐE₄ | AlkLEᵥEᵥᵢ | E₄* | Eₐ |
| 322 | E_{1b}E₄ | AlkLEᵥEᵥᵢ | E₄* | Eₐ |
| 323 | E₁ₐEᵢE₄ | AlkLEᵥEᵥᵢ | E₄* | Eₐ |
| 324 | E_{1b}EᵢE₄ | AlkLEᵥEᵥᵢ | E₄* | Eₐ |
| 325 | E₁ₐEᵢᵢE₄ | AlkLEᵥEᵥᵢ | E₄* | Eₐ |
| 326 | E_{1b}EᵢᵢE₄ | AlkLEᵥEᵥᵢ | E₄* | Eₐ |
| 327 | E₁ₐEᵢᵢE_{iib}E₄ | AlkLEᵥEᵥᵢ | E₄* | Eₐ |
| 328 | E_{1b}EᵢᵢE_{iib}E₄ | AlkLEᵥEᵥᵢ | E₄* | Eₐ |
| 329 | E₁ₐEᵢᵢᵢ | AlkLEᵥEᵥᵢ | E₄* | Eₐ |
| 330 | E_{1b}Eᵢᵢᵢ | AlkLEᵥEᵥᵢ | E₄* | Eₐ |
| 331 | E₁ₐEᵢᵢᵢE₄ | AlkLEᵥEᵥᵢ | E₄* | Eₐ |
| 332 | E_{1b}EᵢᵢᵢE₄ | AlkLEᵥEᵥᵢ | E₄* | Eₐ |
| 333 | E₁ₐEᵢᵥ | AlkLEᵥEᵥᵢ | E₄* | Eₐ |
| 334 | E_{1b}Eᵢᵥ | AlkLEᵥEᵥᵢ | E₄* | Eₐ |
| 335 | E₁ₐEᵢᵥE₄ | AlkLEᵥEᵥᵢ | E₄* | Eₐ |
| 336 | E_{1b}EᵢᵥE₄ | AlkLEᵥEᵥᵢ | E₄* | Eₐ |
| 337 | E₁ₐ | AlkLEᵥEᵥᵢ | E₅ | Eₐ |
| 338 | E_{1b} | AlkLEᵥEᵥᵢ | E₅ | Eₐ |
| 339 | E₁ₐEᵢ | AlkLEᵥEᵥᵢ | E₅ | Eₐ |
| 340 | E_{1b}Eᵢ | AlkLEᵥEᵥᵢ | E₅ | Eₐ |
| 341 | E₁ₐEᵢᵢ | AlkLEᵥEᵥᵢ | E₅ | Eₐ |
| 342 | E_{1b}Eᵢᵢ | AlkLEᵥEᵥᵢ | E₅ | Eₐ |
| 343 | E₁ₐEᵢᵢE_{iib} | AlkLEᵥEᵥᵢ | E₅ | Eₐ |
| 344 | E_{1b}EᵢᵢE_{iib} | AlkLEᵥEᵥᵢ | E₅ | Eₐ |
| 345 | E₁ₐE₄ | AlkLEᵥEᵥᵢ | E₅ | Eₐ |
| 346 | E_{1b}E₄ | AlkLEᵥEᵥᵢ | E₅ | Eₐ |
| 347 | E₁ₐEᵢE₄ | AlkLEᵥEᵥᵢ | E₅ | Eₐ |
| 348 | E_{1b}EᵢE₄ | AlkLEᵥEᵥᵢ | E₅ | Eₐ |
| 349 | E₁ₐEᵢᵢE₄ | AlkLEᵥEᵥᵢ | E₅ | Eₐ |
| 350 | E_{1b}EᵢᵢE₄ | AlkLEᵥEᵥᵢ | E₅ | Eₐ |
| 351 | E₁ₐEᵢᵢE_{iib}E₄ | AlkLEᵥEᵥᵢ | E₅ | Eₐ |
| 352 | E_{1b}EᵢᵢE_{iib}E₄ | AlkLEᵥEᵥᵢ | E₅ | Eₐ |
| 353 | E₁ₐEᵢᵢᵢ | AlkLEᵥEᵥᵢ | E₅ | Eₐ |
| 354 | E_{1b}Eᵢᵢᵢ | AlkLEᵥEᵥᵢ | E₅ | Eₐ |
| 355 | E₁ₐEᵢᵢᵢE₄ | AlkLEᵥEᵥᵢ | E₅ | Eₐ |
| 356 | E_{1b}EᵢᵢᵢE₄ | AlkLEᵥEᵥᵢ | E₅ | Eₐ |
| 357 | E₁ₐEᵢᵥ | AlkLEᵥEᵥᵢ | E₅ | Eₐ |
| 358 | E_{1b}Eᵢᵥ | AlkLEᵥEᵥᵢ | E₅ | Eₐ |
| 359 | E₁ₐEᵢᵥE₄ | AlkLEᵥEᵥᵢ | E₅ | Eₐ |
| 360 | E_{1b}EᵢᵥE₄ | AlkLEᵥEᵥᵢ | E₅ | Eₐ |
| 361 | E₁ₐ | AlkLEᵥEᵥᵢ | E₄*E₅ | Eₐ |
| 362 | E_{1b} | AlkLEᵥEᵥᵢ | E₄*E₅ | Eₐ |
| 363 | E₁ₐEᵢ | AlkLEᵥEᵥᵢ | E₄*E₅ | Eₐ |
| 364 | E_{1b}Eᵢ | AlkLEᵥEᵥᵢ | E₄*E₅ | Eₐ |
| 365 | E₁ₐEᵢᵢ | AlkLEᵥEᵥᵢ | E₄*E₅ | Eₐ |
| 366 | E_{1b}Eᵢᵢ | AlkLEᵥEᵥᵢ | E₄*E₅ | Eₐ |
| 367 | E₁ₐEᵢᵢE_{iib} | AlkLEᵥEᵥᵢ | E₄*E₅ | Eₐ |
| 368 | E_{1b}EᵢᵢE_{iib} | AlkLEᵥEᵥᵢ | E₄*E₅ | Eₐ |
| 369 | E₁ₐE₄ | AlkLEᵥEᵥᵢ | E₄*E₅ | Eₐ |
| 370 | E_{1b}E₄ | AlkLEᵥEᵥᵢ | E₄*E₅ | Eₐ |
| 371 | E₁ₐEᵢE₄ | AlkLEᵥEᵥᵢ | E₄*E₅ | Eₐ |
| 372 | E_{1b}EᵢE₄ | AlkLEᵥEᵥᵢ | E₄*E₅ | Eₐ |
| 373 | E₁ₐEᵢᵢE₄ | AlkLEᵥEᵥᵢ | E₄*E₅ | Eₐ |
| 374 | E_{1b}EᵢᵢE₄ | AlkLEᵥEᵥᵢ | E₄*E₅ | Eₐ |
| 375 | E₁ₐEᵢᵢE_{iib}E₄ | AlkLEᵥEᵥᵢ | E₄*E₅ | Eₐ |
| 376 | E_{1b}EᵢᵢE_{iib}E₄ | AlkLEᵥEᵥᵢ | E₄*E₅ | Eₐ |
| 377 | E₁ₐEᵢᵢᵢ | AlkLEᵥEᵥᵢ | E₄*E₅ | Eₐ |
| 378 | E_{1b}Eᵢᵢᵢ | AlkLEᵥEᵥᵢ | E₄*E₅ | Eₐ |
| 379 | E₁ₐEᵢᵢᵢE₄ | AlkLEᵥEᵥᵢ | E₄*E₅ | Eₐ |
| 380 | E_{1b}EᵢᵢᵢE₄ | AlkLEᵥEᵥᵢ | E₄*E₅ | Eₐ |
| 381 | E₁ₐEᵢᵥ | AlkLEᵥEᵥᵢ | E₄*E₅ | Eₐ |
| 382 | E_{1b}Eᵢᵥ | AlkLEᵥEᵥᵢ | E₄*E₅ | Eₐ |
| 383 | E₁ₐEᵢᵥE₄ | AlkLEᵥEᵥᵢ | E₄*E₅ | Eₐ |
| 384 | E_{1b}EᵢᵥE₄ | AlkLEᵥEᵥᵢ | E₄*E₅ | Eₐ |
| 385 | E₁ₐ | EᵥEᵥᵢ | | E_{b} |
| 386 | E_{1b} | EᵥEᵥᵢ | | E_{b} |
| 387 | E₁ₐEᵢ | EᵥEᵥᵢ | | E_{b} |
| 388 | E_{1b}Eᵢ | EᵥEᵥᵢ | | E_{b} |
| 389 | E₁ₐEᵢᵢ | EᵥEᵥᵢ | | E_{b} |
| 390 | E_{1b}Eᵢᵢ | EᵥEᵥᵢ | | E_{b} |
| 391 | E₁ₐEᵢᵢE_{iib} | EᵥEᵥᵢ | | E_{b} |
| 392 | E_{1b}EᵢᵢE_{iib} | EᵥEᵥᵢ | | E_{b} |
| 393 | E₁ₐE₄ | EᵥEᵥᵢ | | E_{b} |
| 394 | E_{1b}E₄ | EᵥEᵥᵢ | | E_{b} |
| 395 | E₁ₐEᵢE₄ | EᵥEᵥᵢ | | E_{b} |
| 396 | E_{1b}EᵢE₄ | EᵥEᵥᵢ | | E_{b} |
| 397 | E₁ₐEᵢᵢE₄ | EᵥEᵥᵢ | | E_{b} |
| 398 | E_{1b}EᵢᵢE₄ | EᵥEᵥᵢ | | E_{b} |
| 399 | E₁ₐEᵢᵢE_{iib}E₄ | EᵥEᵥᵢ | | E_{b} |
| 400 | E_{1b}EᵢᵢE_{iib}E₄ | EᵥEᵥᵢ | | E_{b} |
| 401 | E₁ₐEᵢᵢᵢ | EᵥEᵥᵢ | | E_{b} |
| 402 | E_{1b}Eᵢᵢᵢ | EᵥEᵥᵢ | | E_{b} |
| 403 | E₁ₐEᵢᵢᵢE₄ | EᵥEᵥᵢ | | E_{b} |
| 404 | E_{1b}EᵢᵢᵢE₄ | EᵥEᵥᵢ | | E_{b} |
| 405 | E₁ₐEᵢᵥ | EᵥEᵥᵢ | | E_{b} |
| 406 | E_{1b}Eᵢᵥ | EᵥEᵥᵢ | | E_{b} |
| 407 | E₁ₐEᵢᵥE₄ | EᵥEᵥᵢ | | E_{b} |
| 408 | E_{1b}EᵢᵥE₄ | EᵥEᵥᵢ | | E_{b} |
| 409 | E₁ₐ | EᵥEᵥᵢ | E₄* | E_{b} |
| 410 | E_{1b} | EᵥEᵥᵢ | E₄* | E_{b} |
| 411 | E₁ₐEᵢ | EᵥEᵥᵢ | E₄* | E_{b} |
| 412 | E_{1b}Eᵢ | EᵥEᵥᵢ | E₄* | E_{b} |
| 413 | E₁ₐEᵢᵢ | EᵥEᵥᵢ | E₄* | E_{b} |
| 414 | E_{1b}Eᵢᵢ | EᵥEᵥᵢ | E₄* | E_{b} |
| 415 | E₁ₐEᵢᵢE_{iib} | EᵥEᵥᵢ | E₄* | E_{b} |
| 416 | E_{1b}EᵢᵢE_{iib} | EᵥEᵥᵢ | E₄* | E_{b} |
| 417 | E₁ₐE₄ | EᵥEᵥᵢ | E₄* | E_{b} |
| 418 | E_{1b}E₄ | EᵥEᵥᵢ | E₄* | E_{b} |
| 419 | E₁ₐEᵢE₄ | EᵥEᵥᵢ | E₄* | E_{b} |
| 420 | E_{1b}EᵢE₄ | EᵥEᵥᵢ | E₄* | E_{b} |
| 421 | E₁ₐEᵢᵢE₄ | EᵥEᵥᵢ | E₄* | E_{b} |
| 422 | E_{1b}EᵢᵢE₄ | EᵥEᵥᵢ | E₄* | E_{b} |
| 423 | E₁ₐEᵢᵢE_{iib}E₄ | EᵥEᵥᵢ | E₄* | E_{b} |
| 424 | E_{1b}EᵢᵢE_{iib}E₄ | EᵥEᵥᵢ | E₄* | E_{b} |
| 425 | E₁ₐEᵢᵢᵢ | EᵥEᵥᵢ | E₄* | E_{b} |
| 426 | E_{1b}Eᵢᵢᵢ | EᵥEᵥᵢ | E₄* | E_{b} |
| 427 | E₁ₐEᵢᵢᵢE₄ | EᵥEᵥᵢ | E₄* | E_{b} |
| 428 | E_{1b}EᵢᵢᵢE₄ | EᵥEᵥᵢ | E₄* | E_{b} |
| 429 | E₁ₐEᵢᵥ | EᵥEᵥᵢ | E₄* | E_{b} |
| 430 | E_{1b}Eᵢᵥ | EᵥEᵥᵢ | E₄* | E_{b} |
| 431 | E₁ₐEᵢᵥE₄ | EᵥEᵥᵢ | E₄* | E_{b} |
| 432 | E_{1b}EᵢᵥE₄ | EᵥEᵥᵢ | E₄* | E_{b} |
| 433 | E₁ₐ | EᵥEᵥᵢ | E₅ | E_{b} |
| 434 | E_{1b} | EᵥEᵥᵢ | E₅ | E_{b} |
| 435 | E₁ₐEᵢ | EᵥEᵥᵢ | E₅ | E_{b} |
| 436 | E_{1b}Eᵢ | EᵥEᵥᵢ | E₅ | E_{b} |
| 437 | E₁ₐEᵢᵢ | EᵥEᵥᵢ | E₅ | E_{b} |
| 438 | E_{1b}Eᵢᵢ | EᵥEᵥᵢ | E₅ | E_{b} |
| 439 | E₁ₐEᵢᵢE_{iib} | EᵥEᵥᵢ | E₅ | E_{b} |
| 440 | E_{1b}EᵢᵢE_{iib} | EᵥEᵥᵢ | E₅ | E_{b} |
| 441 | E₁ₐE₄ | EᵥEᵥᵢ | E₅ | E_{b} |
| 442 | E_{1b}E₄ | EᵥEᵥᵢ | E₅ | E_{b} |
| 443 | E₁ₐEᵢE₄ | EᵥEᵥᵢ | E₅ | E_{b} |
| 444 | E_{1b}EᵢE₄ | EᵥEᵥᵢ | E₅ | E_{b} |
| 445 | E₁ₐEᵢᵢE₄ | EᵥEᵥᵢ | E₅ | E_{b} |
| 446 | E_{1b}EᵢᵢE₄ | EᵥEᵥᵢ | E₅ | E_{b} |
| 447 | E₁ₐEᵢᵢE_{iib}E₄ | EᵥEᵥᵢ | E₅ | E_{b} |
| 448 | E_{1b}EᵢᵢE_{iib}E₄ | EᵥEᵥᵢ | E₅ | E_{b} |
| 449 | E₁ₐEᵢᵢᵢ | EᵥEᵥᵢ | E₅ | E_{b} |
| 450 | E_{1b}Eᵢᵢᵢ | EᵥEᵥᵢ | E₅ | E_{b} |
| 451 | E₁ₐEᵢᵢᵢE₄ | EᵥEᵥᵢ | E₅ | E_{b} |
| 452 | E_{1b}EᵢᵢᵢE₄ | EᵥEᵥᵢ | E₅ | E_{b} |
| 453 | E₁ₐEᵢᵥ | EᵥEᵥᵢ | E₅ | E_{b} |
| 454 | E_{1b}Eᵢᵥ | EᵥEᵥᵢ | E₅ | E_{b} |
| 455 | E₁ₐEᵢᵥE₄ | EᵥEᵥᵢ | E₅ | E_{b} |
| 456 | E_{1b}EᵢᵥE₄ | EᵥEᵥᵢ | E₅ | E_{b} |
| 457 | E₁ₐ | EᵥEᵥᵢ | E₄*E₅ | E_{b} |
| 458 | E_{1b} | EᵥEᵥᵢ | E₄*E₅ | E_{b} |
| 459 | E₁ₐEᵢ | EᵥEᵥᵢ | E₄*E₅ | E_{b} |
| 460 | E_{1b}Eᵢ | EᵥEᵥᵢ | E₄*E₅ | E_{b} |
| 461 | E₁ₐEᵢᵢ | EᵥEᵥᵢ | E₄*E₅ | E_{b} |
| 462 | E_{1b}Eᵢᵢ | EᵥEᵥᵢ | E₄*E₅ | E_{b} |
| 463 | E₁ₐEᵢᵢE_{iib} | EᵥEᵥᵢ | E₄*E₅ | E_{b} |
| 464 | E_{1b}EᵢᵢE_{iib} | EᵥEᵥᵢ | E₄*E₅ | E_{b} |
| 465 | E₁ₐE₄ | EᵥEᵥᵢ | E₄*E₅ | E_{b} |
| 466 | E_{1b}E₄ | EᵥEᵥᵢ | E₄*E₅ | E_{b} |
| 467 | E₁ₐEᵢE₄ | EᵥEᵥᵢ | E₄*E₅ | E_{b} |
| 468 | E_{1b}EᵢE₄ | EᵥEᵥᵢ | E₄*E₅ | E_{b} |
| 469 | E₁ₐEᵢᵢE₄ | EᵥEᵥᵢ | E₄*E₅ | E_{b} |
| 470 | E_{1b}EᵢᵢE₄ | EᵥEᵥᵢ | E₄*E₅ | E_{b} |
| 471 | E₁ₐEᵢᵢE_{iib}E₄ | EᵥEᵥᵢ | E₄*E₅ | E_{b} |
| 472 | E_{1b}EᵢᵢE_{iib}E₄ | EᵥEᵥᵢ | E₄*E₅ | E_{b} |
| 473 | E₁ₐEᵢᵢᵢ | EᵥEᵥᵢ | E₄*E₅ | E_{b} |
| 474 | E_{1b}Eᵢᵢᵢ | EᵥEᵥᵢ | E₄*E₅ | E_{b} |
| 475 | E₁ₐEᵢᵢᵢE₄ | EᵥEᵥᵢ | E₄*E₅ | E_{b} |
| 476 | E_{1b}EᵢᵢᵢE₄ | EᵥEᵥᵢ | E₄*E₅ | E_{b} |
| 477 | E₁ₐEᵢᵥ | EᵥEᵥᵢ | E₄*E₅ | E_{b} |
| 478 | E_{1b}Eᵢᵥ | EᵥEᵥᵢ | E₄*E₅ | E_{b} |
| 479 | E₁ₐEᵢᵥE₄ | EᵥEᵥᵢ | E₄*E₅ | E_{b} |
| 480 | E_{1b}EᵢᵥE₄ | EᵥEᵥᵢ | E₄*E₅ | E_{b} |
| 481 | E₁ₐ | AlkLEᵥEᵥᵢ | | E_{b} |
| 482 | E_{1b} | AlkLEᵥEᵥᵢ | | E_{b} |
| 483 | E₁ₐEᵢ | AlkLEᵥEᵥᵢ | | E_{b} |
| 484 | E_{1b}Eᵢ | AlkLEᵥEᵥᵢ | | E_{b} |
| 485 | E₁ₐEᵢᵢ | AlkLEᵥEᵥᵢ | | E_{b} |
| 486 | E_{1b}Eᵢᵢ | AlkLEᵥEᵥᵢ | | E_{b} |
| 487 | E₁ₐEᵢᵢE_{iib} | AlkLEᵥEᵥᵢ | | E_{b} |
| 488 | E_{1b}EᵢᵢE_{iib} | AlkLEᵥEᵥᵢ | | E_{b} |
| 489 | E₁ₐE₄ | AlkLEᵥEᵥᵢ | | E_{b} |
| 490 | E_{1b}E₄ | AlkLEᵥEᵥᵢ | | E_{b} |
| 491 | E₁ₐEᵢE₄ | AlkLEᵥEᵥᵢ | | E_{b} |
| 492 | E_{1b}EᵢE₄ | AlkLEᵥEᵥᵢ | | E_{b} |
| 493 | E₁ₐEᵢᵢE₄ | AlkLEᵥEᵥᵢ | | E_{b} |
| 494 | E_{1b}EᵢᵢE₄ | AlkLEᵥEᵥᵢ | | E_{b} |
| 495 | E₁ₐEᵢᵢE_{iib}E₄ | AlkLEᵥEᵥᵢ | | E_{b} |
| 496 | E_{1b}EᵢᵢE_{iib}E₄ | AlkLEᵥEᵥᵢ | | E_{b} |
| 497 | E₁ₐEᵢᵢᵢ | AlkLEᵥEᵥᵢ | | E_{b} |
| 498 | E_{1b}Eᵢᵢᵢ | AlkLEᵥEᵥᵢ | | E_{b} |
| 499 | E₁ₐEᵢᵢᵢE₄ | AlkLEᵥEᵥᵢ | | E_{b} |
| 500 | E_{1b}EᵢᵢᵢE₄ | AlkLEᵥEᵥᵢ | | E_{b} |
| 501 | E₁ₐEᵢᵥ | AlkLEᵥEᵥᵢ | | E_{b} |
| 502 | E_{1b}Eᵢᵥ | AlkLEᵥEᵥᵢ | | E_{b} |
| 503 | E₁ₐEᵢᵥE₄ | AlkLEᵥEᵥᵢ | | E_{b} |
| 504 | E_{1b}EᵢᵥE₄ | AlkLEᵥEᵥᵢ | | E_{b} |
| 505 | E₁ₐ | AlkLEᵥEᵥᵢ | E₄* | E_{b} |
| 506 | E_{1b} | AlkLEᵥEᵥᵢ | E₄* | E_{b} |
| 507 | E₁ₐEᵢ | AlkLEᵥEᵥᵢ | E₄* | E_{b} |
| 508 | E_{1b}Eᵢ | AlkLEᵥEᵥᵢ | E₄* | E_{b} |
| 509 | E₁ₐEᵢᵢ | AlkLEᵥEᵥᵢ | E₄* | E_{b} |
| 510 | E_{1b}Eᵢᵢ | AlkLEᵥEᵥᵢ | E₄* | E_{b} |
| 511 | E₁ₐEᵢᵢE_{iib} | AlkLEᵥEᵥᵢ | E₄* | E_{b} |
| 512 | E_{1b}EᵢᵢE_{iib} | AlkLEᵥEᵥᵢ | E₄* | E_{b} |
| 513 | E₁ₐE₄ | AlkLEᵥEᵥᵢ | E₄* | E_{b} |
| 514 | E_{1b}E₄ | AlkLEᵥEᵥᵢ | E₄* | E_{b} |
| 515 | E₁ₐEᵢE₄ | AlkLEᵥEᵥᵢ | E₄* | E_{b} |
| 516 | E_{1b}EᵢE₄ | AlkLEᵥEᵥᵢ | E₄* | E_{b} |
| 517 | E₁ₐEᵢᵢE₄ | AlkLEᵥEᵥᵢ | E₄* | E_{b} |
| 518 | E_{1b}EᵢᵢE₄ | AlkLEᵥEᵥᵢ | E₄* | E_{b} |
| 519 | E₁ₐEᵢᵢE_{iib}E₄ | AlkLEᵥEᵥᵢ | E₄* | E_{b} |
| 520 | E_{1b}EᵢᵢE_{iib}E₄ | AlkLEᵥEᵥᵢ | E₄* | E_{b} |
| 521 | E₁ₐEᵢᵢᵢ | AlkLEᵥEᵥᵢ | E₄* | E_{b} |
| 522 | E_{1b}Eᵢᵢᵢ | AlkLEᵥEᵥᵢ | E₄* | E_{b} |
| 523 | E₁ₐEᵢᵢᵢE₄ | AlkLEᵥEᵥᵢ | E₄* | E_{b} |
| 524 | E_{1b}EᵢᵢᵢE₄ | AlkLEᵥEᵥᵢ | E₄* | E_{b} |
| 525 | E₁ₐEᵢᵥ | AlkLEᵥEᵥᵢ | E₄* | E_{b} |
| 526 | E_{1b}Eᵢᵥ | AlkLEᵥEᵥᵢ | E₄* | E_{b} |
| 527 | E₁ₐEᵢᵥE₄ | AlkLEᵥEᵥᵢ | E₄* | E_{b} |
| 528 | E_{1b}EᵢᵥE₄ | AlkLEᵥEᵥᵢ | E₄* | E_{b} |
| 529 | E₁ₐ | AlkLEᵥEᵥᵢ | E₅ | E_{b} |
| 530 | E_{1b} | AlkLEᵥEᵥᵢ | E₅ | E_{b} |
| 531 | E₁ₐEᵢ | AlkLEᵥEᵥᵢ | E₅ | E_{b} |
| 532 | E_{1b}Eᵢ | AlkLEᵥEᵥᵢ | E₅ | E_{b} |
| 533 | E₁ₐEᵢᵢ | AlkLEᵥEᵥᵢ | E₅ | E_{b} |
| 534 | E_{1b}Eᵢᵢ | AlkLEᵥEᵥᵢ | E₅ | E_{b} |
| 535 | E₁ₐEᵢᵢE_{iib} | AlkLEᵥEᵥᵢ | E₅ | E_{b} |
| 536 | E_{1b}EᵢᵢE_{iib} | AlkLEᵥEᵥᵢ | E₅ | E_{b} |
| 537 | E₁ₐE₄ | AlkLEᵥEᵥᵢ | E₅ | E_{b} |
| 538 | E_{1b}E₄ | AlkLEᵥEᵥᵢ | E₅ | E_{b} |
| 539 | E₁ₐEᵢE₄ | AlkLEᵥEᵥᵢ | E₅ | E_{b} |
| 540 | E_{1b}EᵢE₄ | AlkLEᵥEᵥᵢ | E₅ | E_{b} |
| 541 | E₁ₐEᵢᵢE₄ | AlkLEᵥEᵥᵢ | E₅ | E_{b} |
| 542 | E_{1b}EᵢᵢE₄ | AlkLEᵥEᵥᵢ | E₅ | E_{b} |
| 543 | E₁ₐEᵢᵢE_{iib}E₄ | AlkLEᵥEᵥᵢ | E₅ | E_{b} |
| 544 | E_{1b}EᵢᵢE_{iib}E₄ | AlkLEᵥEᵥᵢ | E₅ | E_{b} |
| 545 | E₁ₐEᵢᵢᵢ | AlkLEᵥEᵥᵢ | E₅ | E_{b} |
| 546 | E_{1b}Eᵢᵢᵢ | AlkLEᵥEᵥᵢ | E₅ | E_{b} |
| 547 | E₁ₐEᵢᵢᵢE₄ | AlkLEᵥEᵥᵢ | E₅ | E_{b} |
| 548 | E_{1b}EᵢᵢᵢE₄ | AlkLEᵥEᵥᵢ | E₅ | E_{b} |
| 549 | E₁ₐEᵢᵥ | AlkLEᵥEᵥᵢ | E₅ | E_{b} |
| 550 | E_{1b}Eᵢᵥ | AlkLEᵥEᵥᵢ | E₅ | E_{b} |
| 551 | E₁ₐEᵢᵥE₄ | AlkLEᵥEᵥᵢ | E₅ | E_{b} |
| 552 | E_{1b}EᵢᵥE₄ | AlkLEᵥEᵥᵢ | E₅ | E_{b} |
| 553 | E₁ₐ | AlkLEᵥEᵥᵢ | E₄*E₅ | E_{b} |
| 554 | E_{1b} | AlkLEᵥEᵥᵢ | E₄*E₅ | E_{b} |
| 555 | E₁ₐEᵢ | AlkLEᵥEᵥᵢ | E₄*E₅ | E_{b} |
| 556 | E_{1b}Eᵢ | AlkLEᵥEᵥᵢ | E₄*E₅ | E_{b} |
| 557 | E₁ₐEᵢᵢ | AlkLEᵥEᵥᵢ | E₄*E₅ | E_{b} |
| 558 | E_{1b}Eᵢᵢ | AlkLEᵥEᵥᵢ | E₄*E₅ | E_{b} |
| 559 | E₁ₐEᵢᵢE_{iib} | AlkLEᵥEᵥᵢ | E₄*E₅ | E_{b} |
| 560 | E_{1b}EᵢᵢE_{iib} | AlkLEᵥEᵥᵢ | E₄*E₅ | E_{b} |
| 561 | E₁ₐE₄ | AlkLEᵥEᵥᵢ | E₄*E₅ | E_{b} |
| 562 | E_{1b}E₄ | AlkLEᵥEᵥᵢ | E₄*E₅ | E_{b} |
| 563 | E₁ₐEᵢE₄ | AlkLEᵥEᵥᵢ | E₄*E₅ | E_{b} |
| 564 | E_{1b}EᵢE₄ | AlkLEᵥEᵥᵢ | E₄*E₅ | E_{b} |
| 565 | E₁ₐEᵢᵢE₄ | AlkLEᵥEᵥᵢ | E₄*E₅ | E_{b} |
| 566 | E_{1b}EᵢᵢE₄ | AlkLEᵥEᵥᵢ | E₄*E₅ | E_{b} |
| 567 | E₁ₐEᵢᵢE_{iib}E₄ | AlkLEᵥEᵥᵢ | E₄*E₅ | E_{b} |
| 568 | E_{1b}EᵢᵢE_{iib}E₄ | AlkLEᵥEᵥᵢ | E₄*E₅ | E_{b} |
| 569 | E₁ₐEᵢᵢᵢ | AlkLEᵥEᵥᵢ | E₄*E₅ | E_{b} |
| 570 | E_{1b}Eᵢᵢᵢ | AlkLEᵥEᵥᵢ | E₄*E₅ | E_{b} |
| 571 | E₁ₐEᵢᵢᵢE₄ | AlkLEᵥEᵥᵢ | E₄*E₅ | E_{b} |
| 572 | E_{1b}EᵢᵢᵢE₄ | AlkLEᵥEᵥᵢ | E₄*E₅ | E_{b} |
| 573 | E₁ₐEᵢᵥ | AlkLEᵥEᵥᵢ | E₄*E₅ | E_{b} |
| 574 | E_{1b}Eᵢᵥ | AlkLEᵥEᵥᵢ | E₄*E₅ | E_{b} |
| 575 | E₁ₐEᵢᵥE₄ | AlkLEᵥEᵥᵢ | E₄*E₅ | E_{b} |
| 576 | E_{1b}EᵢᵥE₄ | AlkLEᵥEᵥᵢ | E₄*E₅ | E_{b} |
| 577 | E₁ₐ | EᵥEᵥᵢ | | E_{d} |
| 578 | E_{1b} | EᵥEᵥᵢ | | E_{d} |
| 579 | E₁ₐEᵢ | EᵥEᵥᵢ | | E_{d} |
| 580 | E_{1b}Eᵢ | EᵥEᵥᵢ | | E_{d} |
| 581 | E₁ₐEᵢᵢ | EᵥEᵥᵢ | | E_{d} |
| 582 | E_{1b}Eᵢᵢ | EᵥEᵥᵢ | | E_{d} |
| 583 | E₁ₐEᵢᵢE_{iib} | EᵥEᵥᵢ | | E_{d} |
| 584 | E_{1b}EᵢᵢE_{iib} | EᵥEᵥᵢ | | E_{d} |
| 585 | E₁ₐE₄ | EᵥEᵥᵢ | | E_{d} |
| 586 | E_{1b}E₄ | EᵥEᵥᵢ | | E_{d} |
| 587 | E₁ₐEᵢE₄ | EᵥEᵥᵢ | | E_{d} |
| 588 | E_{1b}EᵢE₄ | EᵥEᵥᵢ | | E_{d} |
| 589 | E₁ₐEᵢᵢE₄ | EᵥEᵥᵢ | | E_{d} |
| 590 | E_{1b}EᵢᵢE₄ | EᵥEᵥᵢ | | E_{d} |
| 591 | E₁ₐEᵢᵢE_{iib}E₄ | EᵥEᵥᵢ | | E_{d} |
| 592 | E_{1b}EᵢᵢE_{iib}E₄ | EᵥEᵥᵢ | | E_{d} |
| 593 | E₁ₐEᵢᵢᵢ | EᵥEᵥᵢ | | E_{d} |
| 594 | E_{1b}Eᵢᵢᵢ | EᵥEᵥᵢ | | E_{d} |
| 595 | E₁ₐEᵢᵢᵢE₄ | EᵥEᵥᵢ | | E_{d} |
| 596 | E_{1b}EᵢᵢᵢE₄ | EᵥEᵥᵢ | | E_{d} |
| 597 | E₁ₐEᵢᵥ | EᵥEᵥᵢ | | E_{d} |
| 598 | E_{1b}Eᵢᵥ | EᵥEᵥᵢ | | E_{d} |
| 599 | E₁ₐEᵢᵥE₄ | EᵥEᵥᵢ | | E_{d} |
| 600 | E_{1b}EᵢᵥE₄ | EᵥEᵥᵢ | | E_{d} |
| 601 | E₁ₐ | EᵥEᵥᵢ | E₄* | E_{d} |
| 602 | E_{1b} | EᵥEᵥᵢ | E₄* | E_{d} |
| 603 | E₁ₐEᵢ | EᵥEᵥᵢ | E₄* | E_{d} |
| 604 | E_{1b}Eᵢ | EᵥEᵥᵢ | E₄* | E_{d} |
| 605 | E₁ₐEᵢᵢ | EᵥEᵥᵢ | E₄* | E_{d} |
| 606 | E_{1b}Eᵢᵢ | EᵥEᵥᵢ | E₄* | E_{d} |
| 607 | E₁ₐEᵢᵢE_{iib} | EᵥEᵥᵢ | E₄* | E_{d} |
| 608 | E_{1b}EᵢᵢE_{iib} | EᵥEᵥᵢ | E₄* | E_{d} |
| 609 | E₁ₐE₄ | EᵥEᵥᵢ | E₄* | E_{d} |
| 610 | E_{1b}E₄ | EᵥEᵥᵢ | E₄* | E_{d} |
| 611 | E₁ₐEᵢE₄ | EᵥEᵥᵢ | E₄* | E_{d} |
| 612 | E_{1b}EᵢE₄ | EᵥEᵥᵢ | E₄* | E_{d} |
| 613 | E₁ₐEᵢᵢE₄ | EᵥEᵥᵢ | E₄* | E_{d} |
| 614 | E_{1b}EᵢᵢE₄ | EᵥEᵥᵢ | E₄* | E_{d} |
| 615 | E₁ₐEᵢᵢE_{iib}E₄ | EᵥEᵥᵢ | E₄* | E_{d} |
| 616 | E_{1b}EᵢᵢE_{iib}E₄ | EᵥEᵥᵢ | E₄* | E_{d} |
| 617 | E₁ₐEᵢᵢᵢ | EᵥEᵥᵢ | E₄* | E_{d} |
| 618 | E_{1b}Eᵢᵢᵢ | EᵥEᵥᵢ | E₄* | E_{d} |
| 619 | E₁ₐEᵢᵢᵢE₄ | EᵥEᵥᵢ | E₄* | E_{d} |
| 620 | E_{1b}EᵢᵢᵢE₄ | EᵥEᵥᵢ | E₄* | E_{d} |
| 621 | E₁ₐEᵢᵥ | EᵥEᵥᵢ | E₄* | E_{d} |
| 622 | E_{1b}Eᵢᵥ | EᵥEᵥᵢ | E₄* | E_{d} |
| 623 | E₁ₐEᵢᵥE₄ | EᵥEᵥᵢ | E₄* | E_{d} |
| 624 | E_{1b}EᵢᵥE₄ | EᵥEᵥᵢ | E₄* | E_{d} |
| 625 | E₁ₐ | EᵥEᵥᵢ | E₅ | E_{d} |
| 626 | E_{1b} | EᵥEᵥᵢ | E₅ | E_{d} |
| 627 | E₁ₐEᵢ | EᵥEᵥᵢ | E₅ | E_{d} |
| 628 | E_{1b}Eᵢ | EᵥEᵥᵢ | E₅ | E_{d} |
| 629 | E₁ₐEᵢᵢ | EᵥEᵥᵢ | E₅ | E_{d} |
| 630 | E_{1b}Eᵢᵢ | EᵥEᵥᵢ | E₅ | E_{d} |
| 631 | E₁ₐEᵢᵢE_{iib} | EᵥEᵥᵢ | E₅ | E_{d} |
| 632 | E_{1b}EᵢᵢE_{iib} | EᵥEᵥᵢ | E₅ | E_{d} |
| 633 | E₁ₐE₄ | EᵥEᵥᵢ | E₅ | E_{d} |
| 634 | E_{1b}E₄ | EᵥEᵥᵢ | E₅ | E_{d} |
| 635 | E₁ₐEᵢE₄ | EᵥEᵥᵢ | E₅ | E_{d} |
| 636 | E_{1b}EᵢE₄ | EᵥEᵥᵢ | E₅ | E_{d} |
| 637 | E₁ₐEᵢᵢE₄ | EᵥEᵥᵢ | E₅ | E_{d} |
| 638 | E_{1b}EᵢᵢE₄ | EᵥEᵥᵢ | E₅ | E_{d} |
| 639 | E₁ₐEᵢᵢE_{iib}E₄ | EᵥEᵥᵢ | E₅ | E_{d} |
| 640 | E_{1b}EᵢᵢE_{iib}E₄ | EᵥEᵥᵢ | E₅ | E_{d} |
| 641 | E₁ₐEᵢᵢᵢ | EᵥEᵥᵢ | E₅ | E_{d} |
| 642 | E_{1b}Eᵢᵢᵢ | EᵥEᵥᵢ | E₅ | E_{d} |
| 643 | E₁ₐEᵢᵢᵢE₄ | EᵥEᵥᵢ | E₅ | E_{d} |
| 644 | E_{1b}EᵢᵢᵢE₄ | EᵥEᵥᵢ | E₅ | E_{d} |
| 645 | E₁ₐEᵢᵥ | EᵥEᵥᵢ | E₅ | E_{d} |
| 646 | E_{1b}Eᵢᵥ | EᵥEᵥᵢ | E₅ | E_{d} |
| 647 | E₁ₐEᵢᵥE₄ | EᵥEᵥᵢ | E₅ | E_{d} |
| 648 | E_{1b}EᵢᵥE₄ | EᵥEᵥᵢ | E₅ | E_{d} |
| 649 | E₁ₐ | EᵥEᵥᵢ | E₄*E₅ | E_{d} |
| 650 | E_{1b} | EᵥEᵥᵢ | E₄*E₅ | E_{d} |
| 651 | E₁ₐEᵢ | EᵥEᵥᵢ | E₄*E₅ | E_{d} |
| 652 | E_{1b}Eᵢ | EᵥEᵥᵢ | E₄*E₅ | E_{d} |
| 653 | E₁ₐEᵢᵢ | EᵥEᵥᵢ | E₄*E₅ | E_{d} |
| 654 | E_{1b}Eᵢᵢ | EᵥEᵥᵢ | E₄*E₅ | E_{d} |
| 655 | E₁ₐEᵢᵢE_{iib} | EᵥEᵥᵢ | E₄*E₅ | E_{d} |
| 656 | E_{1b}EᵢᵢE_{iib} | EᵥEᵥᵢ | E₄*E₅ | E_{d} |
| 657 | E₁ₐE₄ | EᵥEᵥᵢ | E₄*E₅ | E_{d} |
| 658 | E_{1b}E₄ | EᵥEᵥᵢ | E₄*E₅ | E_{d} |
| 659 | E₁ₐEᵢE₄ | EᵥEᵥᵢ | E₄*E₅ | E_{d} |
| 660 | E_{1b}EᵢE₄ | EᵥEᵥᵢ | E₄*E₅ | E_{d} |
| 661 | E₁ₐEᵢᵢE₄ | EᵥEᵥᵢ | E₄*E₅ | E_{d} |
| 662 | E_{1b}EᵢᵢE₄ | EᵥEᵥᵢ | E₄*E₅ | E_{d} |
| 663 | E₁ₐEᵢᵢE_{iib}E₄ | EᵥEᵥᵢ | E₄*E₅ | E_{d} |
| 664 | E_{1b}EᵢᵢE_{iib}E₄ | EᵥEᵥᵢ | E₄*E₅ | E_{d} |
| 665 | E₁ₐEᵢᵢᵢ | EᵥEᵥᵢ | E₄*E₅ | E_{d} |
| 666 | E_{1b}Eᵢᵢᵢ | EᵥEᵥᵢ | E₄*E₅ | E_{d} |
| 667 | E₁ₐEᵢᵢᵢE₄ | EᵥEᵥᵢ | E₄*E₅ | E_{d} |
| 668 | E_{1b}EᵢᵢᵢE₄ | EᵥEᵥᵢ | E₄*E₅ | E_{d} |
| 669 | E₁ₐEᵢᵥ | EᵥEᵥᵢ | E₄*E₅ | E_{d} |
| 670 | E_{1b}Eᵢᵥ | EᵥEᵥᵢ | E₄*E₅ | E_{d} |
| 671 | E₁ₐEᵢᵥE₄ | EᵥEᵥᵢ | E₄*E₅ | E_{d} |
| 672 | E_{1b}EᵢᵥE₄ | EᵥEᵥᵢ | E₄*E₅ | E_{d} |
| 673 | E₁ₐ | AlkLEᵥEᵥᵢ | | E_{d} |
| 674 | E_{1b} | AlkLEᵥEᵥᵢ | | E_{d} |
| 675 | E₁ₐEᵢ | AlkLEᵥEᵥᵢ | | E_{d} |
| 676 | E_{1b}Eᵢ | AlkLEᵥEᵥᵢ | | E_{d} |
| 677 | E₁ₐEᵢᵢ | AlkLEᵥEᵥᵢ | | E_{d} |
| 678 | E_{1b}Eᵢᵢ | AlkLEᵥEᵥᵢ | | E_{d} |
| 679 | E₁ₐEᵢᵢE_{iib} | AlkLEᵥEᵥᵢ | | E_{d} |
| 680 | E_{1b}EᵢᵢE_{iib} | AlkLEᵥEᵥᵢ | | E_{d} |
| 681 | E₁ₐE₄ | AlkLEᵥEᵥᵢ | | E_{d} |
| 682 | E_{1b}E₄ | AlkLEᵥEᵥᵢ | | E_{d} |
| 683 | E₁ₐEᵢE₄ | AlkLEᵥEᵥᵢ | | E_{d} |
| 684 | E_{1b}EᵢE₄ | AlkLEᵥEᵥᵢ | | E_{d} |
| 685 | E₁ₐEᵢᵢE₄ | AlkLEᵥEᵥᵢ | | E_{d} |
| 686 | E_{1b}EᵢᵢE₄ | AlkLEᵥEᵥᵢ | | E_{d} |
| 687 | E₁ₐEᵢᵢE_{iib}E₄ | AlkLEᵥEᵥᵢ | | E_{d} |
| 688 | E_{1b}EᵢᵢE_{iib}E₄ | AlkLEᵥEᵥᵢ | | E_{d} |
| 689 | E₁ₐEᵢᵢᵢ | AlkLEᵥEᵥᵢ | | E_{d} |
| 690 | E_{1b}Eᵢᵢᵢ | AlkLEᵥEᵥᵢ | | E_{d} |
| 691 | E₁ₐEᵢᵢᵢE₄ | AlkLEᵥEᵥᵢ | | E_{d} |
| 692 | E_{1b}EᵢᵢᵢE₄ | AlkLEᵥEᵥᵢ | | E_{d} |
| 693 | E₁ₐEᵢᵥ | AlkLEᵥEᵥᵢ | | E_{d} |
| 694 | E_{1b}Eᵢᵥ | AlkLEᵥEᵥᵢ | | E_{d} |
| 695 | E₁ₐEᵢᵥE₄ | AlkLEᵥEᵥᵢ | | E_{d} |
| 696 | E_{1b}EᵢᵥE₄ | AlkLEᵥEᵥᵢ | | E_{d} |
| 697 | E₁ₐ | AlkLEᵥEᵥᵢ | E₄* | E_{d} |
| 698 | E_{1b} | AlkLEᵥEᵥᵢ | E₄* | E_{d} |
| 699 | E₁ₐEᵢ | AlkLEᵥEᵥᵢ | E₄* | E_{d} |
| 700 | E_{1b}Eᵢ | AlkLEᵥEᵥᵢ | E₄* | E_{d} |
| 701 | E₁ₐEᵢᵢ | AlkLEᵥEᵥᵢ | E₄* | E_{d} |
| 702 | E_{1b}Eᵢᵢ | AlkLEᵥEᵥᵢ | E₄* | E_{d} |
| 703 | E₁ₐEᵢᵢE_{iib} | AlkLEᵥEᵥᵢ | E₄* | E_{d} |
| 704 | E_{1b}EᵢᵢE_{iib} | AlkLEᵥEᵥᵢ | E₄* | E_{d} |
| 705 | E₁ₐE₄ | AlkLEᵥEᵥᵢ | E₄* | E_{d} |
| 706 | E_{1b}E₄ | AlkLEᵥEᵥᵢ | E₄* | E_{d} |
| 707 | E₁ₐEᵢE₄ | AlkLEᵥEᵥᵢ | E₄* | E_{d} |
| 708 | E_{1b}EᵢE₄ | AlkLEᵥEᵥᵢ | E₄* | E_{d} |
| 709 | E₁ₐEᵢᵢE₄ | AlkLEᵥEᵥᵢ | E₄* | E_{d} |
| 710 | E_{1b}EᵢᵢE₄ | AlkLEᵥEᵥᵢ | E₄* | E_{d} |
| 711 | E₁ₐEᵢᵢE_{iib}E₄ | AlkLEᵥEᵥᵢ | E₄* | E_{d} |
| 712 | E_{1b}EᵢᵢE_{iib}E₄ | AlkLEᵥEᵥᵢ | E₄* | E_{d} |
| 713 | E₁ₐEᵢᵢᵢ | AlkLEᵥEᵥᵢ | E₄* | E_{d} |
| 714 | E_{1b}Eᵢᵢᵢ | AlkLEᵥEᵥᵢ | E₄* | E_{d} |
| 715 | E₁ₐEᵢᵢᵢE₄ | AlkLEᵥEᵥᵢ | E₄* | E_{d} |
| 716 | E_{1b}EᵢᵢᵢE₄ | AlkLEᵥEᵥᵢ | E₄* | E_{d} |
| 717 | E₁ₐEᵢᵥ | AlkLEᵥEᵥᵢ | E₄* | E_{d} |
| 718 | E_{1b}Eᵢᵥ | AlkLEᵥEᵥᵢ | E₄* | E_{d} |
| 719 | E₁ₐEᵢᵥE₄ | AlkLEᵥEᵥᵢ | E₄* | E_{d} |
| 720 | E_{1b}EᵢᵥE₄ | AlkLEᵥEᵥᵢ | E₄* | E_{d} |
| 721 | E₁ₐ | AlkLEᵥEᵥᵢ | E₅ | E_{d} |
| 722 | E_{1b} | AlkLEᵥEᵥᵢ | E₅ | E_{d} |
| 723 | E₁ₐEᵢ | AlkLEᵥEᵥᵢ | E₅ | E_{d} |
| 724 | E_{1b}Eᵢ | AlkLEᵥEᵥᵢ | E₅ | E_{d} |
| 725 | E₁ₐEᵢᵢ | AlkLEᵥEᵥᵢ | E₅ | E_{d} |
| 726 | E_{1b}Eᵢᵢ | AlkLEᵥEᵥᵢ | E₅ | E_{d} |
| 727 | E₁ₐEᵢᵢE_{iib} | AlkLEᵥEᵥᵢ | E₅ | E_{d} |
| 728 | E_{1b}EᵢᵢE_{iib} | AlkLEᵥEᵥᵢ | E₅ | E_{d} |
| 729 | E₁ₐE₄ | AlkLEᵥEᵥᵢ | E₅ | E_{d} |
| 730 | E_{1b}E₄ | AlkLEᵥEᵥᵢ | E₅ | E_{d} |
| 731 | E₁ₐEᵢE₄ | AlkLEᵥEᵥᵢ | E₅ | E_{d} |
| 732 | E_{1b}EᵢE₄ | AlkLEᵥEᵥᵢ | E₅ | E_{d} |
| 733 | E₁ₐEᵢᵢE₄ | AlkLEᵥEᵥᵢ | E₅ | E_{d} |
| 734 | E_{1b}EᵢᵢE₄ | AlkLEᵥEᵥᵢ | E₅ | E_{d} |
| 735 | E₁ₐEᵢᵢE_{iib}E₄ | AlkLEᵥEᵥᵢ | E₅ | E_{d} |
| 736 | E_{1b}EᵢᵢE_{iib}E₄ | AlkLEᵥEᵥᵢ | E₅ | E_{d} |
| 737 | E₁ₐEᵢᵢᵢ | AlkLEᵥEᵥᵢ | E₅ | E_{d} |
| 738 | E_{1b}Eᵢᵢᵢ | AlkLEᵥEᵥᵢ | E₅ | E_{d} |
| 739 | E₁ₐEᵢᵢᵢE₄ | AlkLEᵥEᵥᵢ | E₅ | E_{d} |
| 740 | E_{1b}EᵢᵢᵢE₄ | AlkLEᵥEᵥᵢ | E₅ | E_{d} |
| 741 | E₁ₐEᵢᵥ | AlkLEᵥEᵥᵢ | E₅ | E_{d} |
| 742 | E_{1b}Eᵢᵥ | AlkLEᵥEᵥᵢ | E₅ | E_{d} |
| 743 | E₁ₐEᵢᵥE₄ | AlkLEᵥEᵥᵢ | E₅ | E_{d} |
| 744 | E_{1b}EᵢᵥE₄ | AlkLEᵥEᵥᵢ | E₅ | E_{d} |
| 745 | E₁ₐ | AlkLEᵥEᵥᵢ | E₄*E₅ | E_{d} |
| 746 | E_{1b} | AlkLEᵥEᵥᵢ | E₄*E₅ | E_{d} |
| 747 | E₁ₐEᵢ | AlkLEᵥEᵥᵢ | E₄*E₅ | E_{d} |
| 748 | E_{1b}Eᵢ | AlkLEᵥEᵥᵢ | E₄*E₅ | E_{d} |
| 749 | E₁ₐEᵢᵢ | AlkLEᵥEᵥᵢ | E₄*E₅ | E_{d} |
| 750 | E_{1b}Eᵢᵢ | AlkLEᵥEᵥᵢ | E₄*E₅ | E_{d} |
| 751 | E₁ₐEᵢᵢE_{iib} | AlkLEᵥEᵥᵢ | E₄*E₅ | E_{d} |
| 752 | E_{1b}EᵢᵢE_{iib} | AlkLEᵥEᵥᵢ | E₄*E₅ | E_{d} |
| 753 | E₁ₐE₄ | AlkLEᵥEᵥᵢ | E₄*E₅ | E_{d} |
| 754 | E_{1b}E₄ | AlkLEᵥEᵥᵢ | E₄*E₅ | E_{d} |
| 755 | E₁ₐEᵢE₄ | AlkLEᵥEᵥᵢ | E₄*E₅ | E_{d} |
| 756 | E_{1b}EᵢE₄ | AlkLEᵥEᵥᵢ | E₄*E₅ | E_{d} |
| 757 | E₁ₐEᵢᵢE₄ | AlkLEᵥEᵥᵢ | E₄*E₅ | E_{d} |
| 758 | E_{1b}EᵢᵢE₄ | AlkLEᵥEᵥᵢ | E₄*E₅ | E_{d} |
| 759 | E₁ₐEᵢᵢE_{iib}E₄ | AlkLEᵥEᵥᵢ | E₄*E₅ | E_{d} |
| 760 | E_{1b}EᵢᵢE_{iib}E₄ | AlkLEᵥEᵥᵢ | E₄*E₅ | E_{d} |
| 761 | E₁ₐEᵢᵢᵢ | AlkLEᵥEᵥᵢ | E₄*E₅ | E_{d} |
| 762 | E_{1b}Eᵢᵢᵢ | AlkLEᵥEᵥᵢ | E₄*E₅ | E_{d} |
| 763 | E₁ₐEᵢᵢᵢE₄ | AlkLEᵥEᵥᵢ | E₄*E₅ | E_{d} |
| 764 | E_{1b}EᵢᵢᵢE₄ | AlkLEᵥEᵥᵢ | E₄*E₅ | E_{d} |
| 765 | E₁ₐEᵢᵥ | AlkLEᵥEᵥᵢ | E₄*E₅ | E_{d} |
| 766 | E_{1b}Eᵢᵥ | AlkLEᵥEᵥᵢ | E₄*E₅ | E_{d} |
| 767 | E₁ₐEᵢᵥE₄ | AlkLEᵥEᵥᵢ | E₄*E₅ | E_{d} |
| 768 | E_{1b}EᵢᵥE₄ | AlkLEᵥEᵥᵢ | E₄*E₅ | E_{d} |
| 769 | E₁ₐ | EᵥEᵥᵢ | | Eₑ |
| 770 | E_{1b} | EᵥEᵥᵢ | | Eₑ |
| 771 | E₁ₐEᵢ | EᵥEᵥᵢ | | Eₑ |
| 772 | E_{1b}Eᵢ | EᵥEᵥᵢ | | Eₑ |
| 773 | E₁ₐEᵢᵢ | EᵥEᵥᵢ | | Eₑ |
| 774 | E_{1b}Eᵢᵢ | EᵥEᵥᵢ | | Eₑ |
| 775 | E₁ₐEᵢᵢE_{iib} | EᵥEᵥᵢ | | Eₑ |
| 776 | E_{1b}EᵢᵢE_{iib} | EᵥEᵥᵢ | | Eₑ |
| 777 | E₁ₐE₄ | EᵥEᵥᵢ | | Eₑ |
| 778 | E_{1b}E₄ | EᵥEᵥᵢ | | Eₑ |
| 779 | E₁ₐEᵢE₄ | EᵥEᵥᵢ | | Eₑ |
| 780 | E_{1b}EᵢE₄ | EᵥEᵥᵢ | | Eₑ |
| 781 | E₁ₐEᵢᵢE₄ | EᵥEᵥᵢ | | Eₑ |
| 782 | E_{1b}EᵢᵢE₄ | EᵥEᵥᵢ | | Eₑ |
| 783 | E₁ₐEᵢᵢE_{iib}E₄ | EᵥEᵥᵢ | | Eₑ |
| 784 | E_{1b}EᵢᵢE_{iib}E₄ | EᵥEᵥᵢ | | Eₑ |
| 785 | E₁ₐEᵢᵢᵢ | EᵥEᵥᵢ | | Eₑ |
| 786 | E_{1b}Eᵢᵢᵢ | EᵥEᵥᵢ | | Eₑ |
| 787 | E₁ₐEᵢᵢᵢE₄ | EᵥEᵥᵢ | | Eₑ |
| 788 | E_{1b}EᵢᵢᵢE₄ | EᵥEᵥᵢ | | Eₑ |
| 789 | E₁ₐEᵢᵥ | EᵥEᵥᵢ | | Eₑ |
| 790 | E_{1b}Eᵢᵥ | EᵥEᵥᵢ | | Eₑ |
| 791 | E₁ₐEᵢᵥE₄ | EᵥEᵥᵢ | | Eₑ |
| 792 | E_{1b}EᵢᵥE₄ | EᵥEᵥᵢ | | Eₑ |
| 793 | E₁ₐ | EᵥEᵥᵢ | E₄* | Eₑ |
| 794 | E_{1b} | EᵥEᵥᵢ | E₄* | Eₑ |
| 795 | E₁ₐEᵢ | EᵥEᵥᵢ | E₄* | Eₑ |
| 796 | E_{1b}Eᵢ | EᵥEᵥᵢ | E₄* | Eₑ |
| 797 | E₁ₐEᵢᵢ | EᵥEᵥᵢ | E₄* | Eₑ |
| 798 | E_{1b}Eᵢᵢ | EᵥEᵥᵢ | E₄* | Eₑ |
| 799 | E₁ₐEᵢᵢE_{iib} | EᵥEᵥᵢ | E₄* | Eₑ |
| 800 | E_{1b}EᵢᵢE_{iib} | EᵥEᵥᵢ | E₄* | Eₑ |
| 801 | E₁ₐE₄ | EᵥEᵥᵢ | E₄* | Eₑ |
| 802 | E_{1b}E₄ | EᵥEᵥᵢ | E₄* | Eₑ |
| 803 | E₁ₐEᵢE₄ | EᵥEᵥᵢ | E₄* | Eₑ |
| 804 | E_{1b}EᵢE₄ | EᵥEᵥᵢ | E₄* | Eₑ |
| 805 | E₁ₐEᵢᵢE₄ | EᵥEᵥᵢ | E₄* | Eₑ |
| 806 | E_{1b}EᵢᵢE₄ | EᵥEᵥᵢ | E₄* | Eₑ |
| 807 | E₁ₐEᵢᵢE_{iib}E₄ | EᵥEᵥᵢ | E₄* | Eₑ |
| 808 | E_{1b}EᵢᵢE_{iib}E₄ | EᵥEᵥᵢ | E₄* | Eₑ |
| 809 | E₁ₐEᵢᵢᵢ | EᵥEᵥᵢ | E₄* | Eₑ |
| 810 | E_{1b}Eᵢᵢᵢ | EᵥEᵥᵢ | E₄* | Eₑ |
| 811 | E₁ₐEᵢᵢᵢE₄ | EᵥEᵥᵢ | E₄* | Eₑ |
| 812 | E_{1b}EᵢᵢᵢE₄ | EᵥEᵥᵢ | E₄* | Eₑ |
| 813 | E₁ₐEᵢᵥ | EᵥEᵥᵢ | E₄* | Eₑ |
| 814 | E_{1b}Eᵢᵥ | EᵥEᵥᵢ | E₄* | Eₑ |
| 815 | E₁ₐEᵢᵥE₄ | EᵥEᵥᵢ | E₄* | Eₑ |
| 816 | E_{1b}EᵢᵥE₄ | EᵥEᵥᵢ | E₄* | Eₑ |
| 817 | E₁ₐ | EᵥEᵥᵢ | E₅ | Eₑ |
| 818 | E_{1b} | EᵥEᵥᵢ | E₅ | Eₑ |
| 819 | E₁ₐEᵢ | EᵥEᵥᵢ | E₅ | Eₑ |
| 820 | E_{1b}Eᵢ | EᵥEᵥᵢ | E₅ | Eₑ |
| 821 | E₁ₐEᵢᵢ | EᵥEᵥᵢ | E₅ | Eₑ |
| 822 | E_{1b}Eᵢᵢ | EᵥEᵥᵢ | E₅ | Eₑ |
| 823 | E₁ₐEᵢᵢE_{iib} | EᵥEᵥᵢ | E₅ | Eₑ |
| 824 | E_{1b}EᵢᵢE_{iib} | EᵥEᵥᵢ | E₅ | Eₑ |
| 825 | E₁ₐE₄ | EᵥEᵥᵢ | E₅ | Eₑ |
| 826 | E_{1b}E₄ | EᵥEᵥᵢ | E₅ | Eₑ |
| 827 | E₁ₐEᵢE₄ | EᵥEᵥᵢ | E₅ | Eₑ |
| 828 | E_{1b}EᵢE₄ | EᵥEᵥᵢ | E₅ | Eₑ |
| 829 | E₁ₐEᵢᵢE₄ | EᵥEᵥᵢ | E₅ | Eₑ |
| 830 | E_{1b}EᵢᵢE₄ | EᵥEᵥᵢ | E₅ | Eₑ |
| 831 | E₁ₐEᵢᵢE_{iib}E₄ | EᵥEᵥᵢ | E₅ | Eₑ |
| 832 | E_{1b}EᵢᵢE_{iib}E₄ | EᵥEᵥᵢ | E₅ | Eₑ |
| 833 | E₁ₐEᵢᵢᵢ | EᵥEᵥᵢ | E₅ | Eₑ |
| 834 | E_{1b}Eᵢᵢᵢ | EᵥEᵥᵢ | E₅ | Eₑ |
| 835 | E₁ₐEᵢᵢᵢE₄ | EᵥEᵥᵢ | E₅ | Eₑ |
| 836 | E_{1b}EᵢᵢᵢE₄ | EᵥEᵥᵢ | E₅ | Eₑ |
| 837 | E₁ₐEᵢᵥ | EᵥEᵥᵢ | E₅ | Eₑ |
| 838 | E_{1b}Eᵢᵥ | EᵥEᵥᵢ | E₅ | Eₑ |
| 839 | E₁ₐEᵢᵥE₄ | EᵥEᵥᵢ | E₅ | Eₑ |
| 840 | E_{1b}EᵢᵥE₄ | EᵥEᵥᵢ | E₅ | Eₑ |
| 841 | E₁ₐ | EᵥEᵥᵢ | E₄*E₅ | Eₑ |
| 842 | E_{1b} | EᵥEᵥᵢ | E₄*E₅ | Eₑ |
| 843 | E₁ₐEᵢ | EᵥEᵥᵢ | E₄*E₅ | Eₑ |
| 844 | E_{1b}Eᵢ | EᵥEᵥᵢ | E₄*E₅ | Eₑ |
| 845 | E₁ₐEᵢᵢ | EᵥEᵥᵢ | E₄*E₅ | Eₑ |
| 846 | E_{1b}Eᵢᵢ | EᵥEᵥᵢ | E₄*E₅ | Eₑ |
| 847 | E₁ₐEᵢᵢE_{iib} | EᵥEᵥᵢ | E₄*E₅ | Eₑ |
| 848 | E_{1b}EᵢᵢE_{iib} | EᵥEᵥᵢ | E₄*E₅ | Eₑ |
| 849 | E₁ₐE₄ | EᵥEᵥᵢ | E₄*E₅ | Eₑ |
| 850 | E_{1b}E₄ | EᵥEᵥᵢ | E₄*E₅ | Eₑ |
| 851 | E₁ₐEᵢE₄ | EᵥEᵥᵢ | E₄*E₅ | Eₑ |
| 852 | E_{1b}EᵢE₄ | EᵥEᵥᵢ | E₄*E₅ | Eₑ |
| 853 | E₁ₐEᵢᵢE₄ | EᵥEᵥᵢ | E₄*E₅ | Eₑ |
| 854 | E_{1b}EᵢᵢE₄ | EᵥEᵥᵢ | E₄*E₅ | Eₑ |
| 855 | E₁ₐEᵢᵢE_{iib}E₄ | EᵥEᵥᵢ | E₄*E₅ | Eₑ |
| 856 | E_{1b}EᵢᵢE_{iib}E₄ | EᵥEᵥᵢ | E₄*E₅ | Eₑ |
| 857 | E₁ₐEᵢᵢᵢ | EᵥEᵥᵢ | E₄*E₅ | Eₑ |
| 858 | E_{1b}Eᵢᵢᵢ | EᵥEᵥᵢ | E₄*E₅ | Eₑ |
| 859 | E₁ₐEᵢᵢᵢE₄ | EᵥEᵥᵢ | E₄*E₅ | Eₑ |
| 860 | E_{1b}EᵢᵢᵢE₄ | EᵥEᵥᵢ | E₄*E₅ | Eₑ |
| 861 | E₁ₐEᵢᵥ | EᵥEᵥᵢ | E₄*E₅ | Eₑ |
| 862 | E_{1b}Eᵢᵥ | EᵥEᵥᵢ | E₄*E₅ | Eₑ |
| 863 | E₁ₐEᵢᵥE₄ | EᵥEᵥᵢ | E₄*E₅ | Eₑ |
| 864 | E_{1b}EᵢᵥE₄ | EᵥEᵥᵢ | E₄*E₅ | Eₑ |
| 865 | E₁ₐ | AlkLEᵥEᵥᵢ | | Eₑ |
| 866 | E_{1b} | AlkLEᵥEᵥᵢ | | Eₑ |
| 867 | E₁ₐEᵢ | AlkLEᵥEᵥᵢ | | Eₑ |
| 868 | E_{1b}Eᵢ | AlkLEᵥEᵥᵢ | | Eₑ |
| 869 | E₁ₐEᵢᵢ | AlkLEᵥEᵥᵢ | | Eₑ |
| 870 | E_{1b}Eᵢᵢ | AlkLEᵥEᵥᵢ | | Eₑ |
| 871 | E₁ₐEᵢᵢE_{iib} | AlkLEᵥEᵥᵢ | | Eₑ |
| 872 | E_{1b}EᵢᵢE_{iib} | AlkLEᵥEᵥᵢ | | Eₑ |
| 873 | E₁ₐE₄ | AlkLEᵥEᵥᵢ | | Eₑ |
| 874 | E_{1b}E₄ | AlkLEᵥEᵥᵢ | | Eₑ |
| 875 | E₁ₐEᵢE₄ | AlkLEᵥEᵥᵢ | | Eₑ |
| 876 | E_{1b}EᵢE₄ | AlkLEᵥEᵥᵢ | | Eₑ |
| 877 | E₁ₐEᵢᵢE₄ | AlkLEᵥEᵥᵢ | | Eₑ |
| 878 | E_{1b}EᵢᵢE₄ | AlkLEᵥEᵥᵢ | | Eₑ |
| 879 | E₁ₐEᵢᵢE_{iib}E₄ | AlkLEᵥEᵥᵢ | | Eₑ |
| 880 | E_{1b}EᵢᵢE_{iib}E₄ | AlkLEᵥEᵥᵢ | | Eₑ |
| 881 | E₁ₐEᵢᵢᵢ | AlkLEᵥEᵥᵢ | | Eₑ |
| 882 | E_{1b}Eᵢᵢᵢ | AlkLEᵥEᵥᵢ | | Eₑ |
| 883 | E₁ₐEᵢᵢᵢE₄ | AlkLEᵥEᵥᵢ | | Eₑ |
| 884 | E_{1b}EᵢᵢᵢE₄ | AlkLEᵥEᵥᵢ | | Eₑ |
| 885 | E₁ₐEᵢᵥ | AlkLEᵥEᵥᵢ | | Eₑ |
| 886 | E_{1b}Eᵢᵥ | AlkLEᵥEᵥᵢ | | Eₑ |
| 887 | E₁ₐEᵢᵥE₄ | AlkLEᵥEᵥᵢ | | Eₑ |
| 888 | E_{1b}EᵢᵥE₄ | AlkLEᵥEᵥᵢ | | Eₑ |
| 889 | E₁ₐ | AlkLEᵥEᵥᵢ | E₄* | Eₑ |
| 890 | E_{1b} | AlkLEᵥEᵥᵢ | E₄* | Eₑ |
| 891 | E₁ₐEᵢ | AlkLEᵥEᵥᵢ | E₄* | Eₑ |
| 892 | E_{1b}Eᵢ | AlkLEᵥEᵥᵢ | E₄* | Eₑ |
| 893 | E₁ₐEᵢᵢ | AlkLEᵥEᵥᵢ | E₄* | Eₑ |
| 894 | E_{1b}Eᵢᵢ | AlkLEᵥEᵥᵢ | E₄* | Eₑ |
| 895 | E₁ₐEᵢᵢE_{iib} | AlkLEᵥEᵥᵢ | E₄* | Eₑ |
| 896 | E_{1b}EᵢᵢE_{iib} | AlkLEᵥEᵥᵢ | E₄* | Eₑ |
| 897 | E₁ₐE₄ | AlkLEᵥEᵥᵢ | E₄* | Eₑ |
| 898 | E_{1b}E₄ | AlkLEᵥEᵥᵢ | E₄* | Eₑ |
| 899 | E₁ₐEᵢE₄ | AlkLEᵥEᵥᵢ | E₄* | Eₑ |
| 900 | E_{1b}EᵢE₄ | AlkLEᵥEᵥᵢ | E₄* | Eₑ |
| 901 | E₁ₐEᵢᵢE₄ | AlkLEᵥEᵥᵢ | E₄* | Eₑ |
| 902 | E_{1b}EᵢᵢE₄ | AlkLEᵥEᵥᵢ | E₄* | Eₑ |
| 903 | E₁ₐEᵢᵢE_{iib}E₄ | AlkLEᵥEᵥᵢ | E₄* | Eₑ |
| 904 | E_{1b}EᵢᵢE_{iib}E₄ | AlkLEᵥEᵥᵢ | E₄* | Eₑ |
| 905 | E₁ₐEᵢᵢᵢ | AlkLEᵥEᵥᵢ | E₄* | Eₑ |
| 906 | E_{1b}Eᵢᵢᵢ | AlkLEᵥEᵥᵢ | E₄* | Eₑ |
| 907 | E₁ₐEᵢᵢᵢE₄ | AlkLEᵥEᵥᵢ | E₄* | Eₑ |
| 908 | E_{1b}EᵢᵢᵢE₄ | AlkLEᵥEᵥᵢ | E₄* | Eₑ |
| 909 | E₁ₐEᵢᵥ | AlkLEᵥEᵥᵢ | E₄* | Eₑ |
| 910 | E_{1b}Eᵢᵥ | AlkLEᵥEᵥᵢ | E₄* | Eₑ |
| 911 | E₁ₐEᵢᵥE₄ | AlkLEᵥEᵥᵢ | E₄* | Eₑ |
| 912 | E_{1b}EᵢᵥE₄ | AlkLEᵥEᵥᵢ | E₄* | Eₑ |
| 913 | E₁ₐ | AlkLEᵥEᵥᵢ | E₅ | Eₑ |
| 914 | E_{1b} | AlkLEᵥEᵥᵢ | E₅ | Eₑ |
| 915 | E₁ₐEᵢ | AlkLEᵥEᵥᵢ | E₅ | Eₑ |
| 916 | E_{1b}Eᵢ | AlkLEᵥEᵥᵢ | E₅ | Eₑ |
| 917 | E₁ₐEᵢᵢ | AlkLEᵥEᵥᵢ | E₅ | Eₑ |
| 918 | E_{1b}Eᵢᵢ | AlkLEᵥEᵥᵢ | E₅ | Eₑ |
| 919 | E₁ₐEᵢᵢE_{iib} | AlkLEᵥEᵥᵢ | E₅ | Eₑ |
| 920 | E_{1b}EᵢᵢE_{iib} | AlkLEᵥEᵥᵢ | E₅ | Eₑ |
| 921 | E₁ₐE₄ | AlkLEᵥEᵥᵢ | E₅ | Eₑ |
| 922 | E_{1b}E₄ | AlkLEᵥEᵥᵢ | E₅ | Eₑ |
| 923 | E₁ₐEᵢE₄ | AlkLEᵥEᵥᵢ | E₅ | Eₑ |
| 924 | E_{1b}EᵢE₄ | AlkLEᵥEᵥᵢ | E₅ | Eₑ |
| 925 | E₁ₐEᵢᵢE₄ | AlkLEᵥEᵥᵢ | E₅ | Eₑ |
| 926 | E_{1b}EᵢᵢE₄ | AlkLEᵥEᵥᵢ | E₅ | Eₑ |
| 927 | E₁ₐEᵢᵢE_{iib}E₄ | AlkLEᵥEᵥᵢ | E₅ | Eₑ |
| 928 | E_{1b}EᵢᵢE_{iib}E₄ | AlkLEᵥEᵥᵢ | E₅ | Eₑ |
| 929 | E₁ₐEᵢᵢᵢ | AlkLEᵥEᵥᵢ | E₅ | Eₑ |
| 930 | E_{1b}Eᵢᵢᵢ | AlkLEᵥEᵥᵢ | E₅ | Eₑ |
| 931 | E₁ₐEᵢᵢᵢE₄ | AlkLEᵥEᵥᵢ | E₅ | Eₑ |
| 932 | E_{1b}EᵢᵢᵢE₄ | AlkLEᵥEᵥᵢ | E₅ | Eₑ |
| 933 | E₁ₐEᵢᵥ | AlkLEᵥEᵥᵢ | E₅ | Eₑ |
| 934 | E_{1b}Eᵢᵥ | AlkLEᵥEᵥᵢ | E₅ | Eₑ |
| 935 | E₁ₐEᵢᵥE₄ | AlkLEᵥEᵥᵢ | E₅ | Eₑ |
| 936 | E_{1b}EᵢᵥE₄ | AlkLEᵥEᵥᵢ | E₅ | Eₑ |
| 937 | E₁ₐ | AlkLEᵥEᵥᵢ | E₄*E₅ | Eₑ |
| 938 | E_{1b} | AlkLEᵥEᵥᵢ | E₄*E₅ | Eₑ |
| 939 | E₁ₐEᵢ | AlkLEᵥEᵥᵢ | E₄*E₅ | Eₑ |
| 940 | E_{1b}Eᵢ | AlkLEᵥEᵥᵢ | E₄*E₅ | Eₑ |
| 941 | E₁ₐEᵢᵢ | AlkLEᵥEᵥᵢ | E₄*E₅ | Eₑ |
| 942 | E_{1b}Eᵢᵢ | AlkLEᵥEᵥᵢ | E₄*E₅ | Eₑ |
| 943 | E₁ₐEᵢᵢE_{iib} | AlkLEᵥEᵥᵢ | E₄*E₅ | Eₑ |
| 944 | E_{1b}EᵢᵢE_{iib} | AlkLEᵥEᵥᵢ | E₄*E₅ | Eₑ |
| 945 | E₁ₐE₄ | AlkLEᵥEᵥᵢ | E₄*E₅ | Eₑ |
| 946 | E_{1b}E₄ | AlkLEᵥEᵥᵢ | E₄*E₅ | Eₑ |
| 947 | E₁ₐEᵢE₄ | AlkLEᵥEᵥᵢ | E₄*E₅ | Eₑ |
| 948 | E_{1b}EᵢE₄ | AlkLEᵥEᵥᵢ | E₄*E₅ | Eₑ |
| 949 | E₁ₐEᵢᵢE₄ | AlkLEᵥEᵥᵢ | E₄*E₅ | Eₑ |
| 950 | E_{1b}EᵢᵢE₄ | AlkLEᵥEᵥᵢ | E₄*E₅ | Eₑ |
| 951 | E₁ₐEᵢᵢE_{iib}E₄ | AlkLEᵥEᵥᵢ | E₄*E₅ | Eₑ |
| 952 | E_{1b}EᵢᵢE_{iib}E₄ | AlkLEᵥEᵥᵢ | E₄*E₅ | Eₑ |
| 953 | E₁ₐEᵢᵢᵢ | AlkLEᵥEᵥᵢ | E₄*E₅ | Eₑ |
| 954 | E_{1b}Eᵢᵢᵢ | AlkLEᵥEᵥᵢ | E₄*E₅ | Eₑ |
| 955 | E₁ₐEᵢᵢᵢE₄ | AlkLEᵥEᵥᵢ | E₄*E₅ | Eₑ |
| 956 | E_{1b}EᵢᵢᵢE₄ | AlkLEᵥEᵥᵢ | E₄*E₅ | Eₑ |
| 957 | E₁ₐEᵢᵥ | AlkLEᵥEᵥᵢ | E₄*E₅ | Eₑ |
| 958 | E_{1b}Eᵢᵥ | AlkLEᵥEᵥᵢ | E₄*E₅ | Eₑ |
| 959 | E₁ₐEᵢᵥE₄ | AlkLEᵥEᵥᵢ | E₄*E₅ | Eₑ |
| 960 | E_{1b}EᵢᵥE₄ | AlkLEᵥEᵥᵢ | E₄*E₅ | Eₑ |
| 961 | E₁ₐ | EᵥEᵥᵢ | | E_{f} |
| 962 | E_{1b} | EᵥEᵥᵢ | | E_{f} |
| 963 | E₁ₐEᵢ | EᵥEᵥᵢ | | E_{f} |
| 964 | E_{1b}Eᵢ | EᵥEᵥᵢ | | E_{f} |
| 965 | E₁ₐEᵢᵢ | EᵥEᵥᵢ | | E_{f} |
| 966 | E_{1b}Eᵢᵢ | EᵥEᵥᵢ | | E_{f} |
| 967 | E₁ₐEᵢᵢE_{iib} | EᵥEᵥᵢ | | E_{f} |
| 968 | E_{1b}EᵢᵢE_{iib} | EᵥEᵥᵢ | | E_{f} |
| 969 | E₁ₐE₄ | EᵥEᵥᵢ | | E_{f} |
| 970 | E_{1b}E₄ | EᵥEᵥᵢ | | E_{f} |
| 971 | E₁ₐEᵢE₄ | EᵥEᵥᵢ | | E_{f} |
| 972 | E_{1b}EᵢE₄ | EᵥEᵥᵢ | | E_{f} |
| 973 | E₁ₐEᵢᵢE₄ | EᵥEᵥᵢ | | E_{f} |
| 974 | E_{1b}EᵢᵢE₄ | EᵥEᵥᵢ | | E_{f} |
| 975 | E₁ₐEᵢᵢE_{iib}E₄ | EᵥEᵥᵢ | | E_{f} |
| 976 | E_{1b}EᵢᵢE_{iib}E₄ | EᵥEᵥᵢ | | E_{f} |
| 977 | E₁ₐEᵢᵢᵢ | EᵥEᵥᵢ | | E_{f} |
| 978 | E_{1b}Eᵢᵢᵢ | EᵥEᵥᵢ | | E_{f} |
| 979 | E₁ₐEᵢᵢᵢE₄ | EᵥEᵥᵢ | | E_{f} |
| 980 | E_{1b}EᵢᵢᵢE₄ | EᵥEᵥᵢ | | E_{f} |
| 981 | E₁ₐEᵢᵥ | EᵥEᵥᵢ | | E_{f} |
| 982 | E_{1b}Eᵢᵥ | EᵥEᵥᵢ | | E_{f} |
| 983 | E₁ₐEᵢᵥE₄ | EᵥEᵥᵢ | | E_{f} |
| 984 | E_{1b}EᵢᵥE₄ | EᵥEᵥᵢ | | E_{f} |
| 985 | E₁ₐ | EᵥEᵥᵢ | E₄* | E_{f} |
| 986 | E_{1b} | EᵥEᵥᵢ | E₄* | E_{f} |
| 987 | E₁ₐEᵢ | EᵥEᵥᵢ | E₄* | E_{f} |
| 988 | E_{1b}Eᵢ | EᵥEᵥᵢ | E₄* | E_{f} |
| 989 | E₁ₐEᵢᵢ | EᵥEᵥᵢ | E₄* | E_{f} |
| 990 | E_{1b}Eᵢᵢ | EᵥEᵥᵢ | E₄* | E_{f} |
| 991 | E₁ₐEᵢᵢE_{iib} | EᵥEᵥᵢ | E₄* | E_{f} |
| 992 | E_{1b}EᵢᵢE_{iib} | EᵥEᵥᵢ | E₄* | E_{f} |
| 993 | E₁ₐE₄ | EᵥEᵥᵢ | E₄* | E_{f} |
| 994 | E_{1b}E₄ | EᵥEᵥᵢ | E₄* | E_{f} |
| 995 | E₁ₐEᵢE₄ | EᵥEᵥᵢ | E₄* | E_{f} |
| 996 | E_{1b}EᵢE₄ | EᵥEᵥᵢ | E₄* | E_{f} |
| 997 | E₁ₐEᵢᵢE₄ | EᵥEᵥᵢ | E₄* | E_{f} |
| 998 | E_{1b}EᵢᵢE₄ | EᵥEᵥᵢ | E₄* | E_{f} |
| 999 | E₁ₐEᵢᵢE_{iib}E₄ | EᵥEᵥᵢ | E₄* | E_{f} |
| 1000 | E_{1b}EᵢᵢE_{iib}E₄ | EᵥEᵥᵢ | E₄* | E_{f} |
| 1001 | E₁ₐEᵢᵢᵢ | EᵥEᵥᵢ | E₄* | E_{f} |
| 1002 | E_{1b}Eᵢᵢᵢ | EᵥEᵥᵢ | E₄* | E_{f} |
| 1003 | E₁ₐEᵢᵢᵢE₄ | EᵥEᵥᵢ | E₄* | E_{f} |
| 1004 | E_{1b}EᵢᵢᵢE₄ | EᵥEᵥᵢ | E₄* | E_{f} |
| 1005 | E₁ₐEᵢᵥ | EᵥEᵥᵢ | E₄* | E_{f} |
| 1006 | E_{1b}Eᵢᵥ | EᵥEᵥᵢ | E₄* | E_{f} |
| 1007 | E₁ₐEᵢᵥE₄ | EᵥEᵥᵢ | E₄* | E_{f} |
| 1008 | E_{1b}EᵢᵥE₄ | EᵥEᵥᵢ | E₄* | E_{f} |
| 1009 | E₁ₐ | EᵥEᵥᵢ | E₅ | E_{f} |
| 1010 | E_{1b} | EᵥEᵥᵢ | E₅ | E_{f} |
| 1011 | E₁ₐEᵢ | EᵥEᵥᵢ | E₅ | E_{f} |
| 1012 | E_{1b}Eᵢ | EᵥEᵥᵢ | E₅ | E_{f} |
| 1013 | E₁ₐEᵢᵢ | EᵥEᵥᵢ | E₅ | E_{f} |
| 1014 | E_{1b}Eᵢᵢ | EᵥEᵥᵢ | E₅ | E_{f} |
| 1015 | E₁ₐEᵢᵢE_{iib} | EᵥEᵥᵢ | E₅ | E_{f} |
| 1016 | E_{1b}EᵢᵢE_{iib} | EᵥEᵥᵢ | E₅ | E_{f} |
| 1017 | E₁ₐE₄ | EᵥEᵥᵢ | E₅ | E_{f} |
| 1018 | E_{1b}E₄ | EᵥEᵥᵢ | E₅ | E_{f} |
| 1019 | E₁ₐEᵢE₄ | EᵥEᵥᵢ | E₅ | E_{f} |
| 1020 | E_{1b}EᵢE₄ | EᵥEᵥᵢ | E₅ | E_{f} |
| 1021 | E₁ₐEᵢᵢE₄ | EᵥEᵥᵢ | E₅ | E_{f} |
| 1022 | E_{1b}EᵢᵢE₄ | EᵥEᵥᵢ | E₅ | E_{f} |
| 1023 | E₁ₐEᵢᵢE_{iib}E₄ | EᵥEᵥᵢ | E₅ | E_{f} |
| 1024 | E_{1b}EᵢᵢE_{iib}E₄ | EᵥEᵥᵢ | E₅ | E_{f} |
| 1025 | E₁ₐEᵢᵢᵢ | EᵥEᵥᵢ | E₅ | E_{f} |
| 1026 | E_{1b}Eᵢᵢᵢ | EᵥEᵥᵢ | E₅ | E_{f} |
| 1027 | E₁ₐEᵢᵢᵢE₄ | EᵥEᵥᵢ | E₅ | E_{f} |
| 1028 | E_{1b}EᵢᵢᵢE₄ | EᵥEᵥᵢ | E₅ | E_{f} |
| 1029 | E₁ₐEᵢᵥ | EᵥEᵥᵢ | E₅ | E_{f} |
| 1030 | E_{1b}Eᵢᵥ | EᵥEᵥᵢ | E₅ | E_{f} |
| 1031 | E₁ₐEᵢᵥE₄ | EᵥEᵥᵢ | E₅ | E_{f} |
| 1032 | E_{1b}EᵢᵥE₄ | EᵥEᵥᵢ | E₅ | E_{f} |
| 1033 | E₁ₐ | EᵥEᵥᵢ | E₄*E₅ | E_{f} |
| 1034 | E_{1b} | EᵥEᵥᵢ | E₄*E₅ | E_{f} |
| 1035 | E₁ₐEᵢ | EᵥEᵥᵢ | E₄*E₅ | E_{f} |
| 1036 | E_{1b}Eᵢ | EᵥEᵥᵢ | E₄*E₅ | E_{f} |
| 1037 | E₁ₐEᵢᵢ | EᵥEᵥᵢ | E₄*E₅ | E_{f} |
| 1038 | E_{1b}Eᵢᵢ | EᵥEᵥᵢ | E₄*E₅ | E_{f} |
| 1039 | E₁ₐEᵢᵢE_{iib} | EᵥEᵥᵢ | E₄*E₅ | E_{f} |
| 1040 | E_{1b}EᵢᵢE_{iib} | EᵥEᵥᵢ | E₄*E₅ | E_{f} |
| 1041 | E₁ₐE₄ | EᵥEᵥᵢ | E₄*E₅ | E_{f} |
| 1042 | E_{1b}E₄ | EᵥEᵥᵢ | E₄*E₅ | E_{f} |
| 1043 | E₁ₐEᵢE₄ | EᵥEᵥᵢ | E₄*E₅ | E_{f} |
| 1044 | E_{1b}EᵢE₄ | EᵥEᵥᵢ | E₄*E₅ | E_{f} |
| 1045 | E₁ₐEᵢᵢE₄ | EᵥEᵥᵢ | E₄*E₅ | E_{f} |
| 1046 | E_{1b}EᵢᵢE₄ | EᵥEᵥᵢ | E₄*E₅ | E_{f} |
| 1047 | E₁ₐEᵢᵢE_{iib}E₄ | EᵥEᵥᵢ | E₄*E₅ | E_{f} |
| 1048 | E_{1b}EᵢᵢE_{iib}E₄ | EᵥEᵥᵢ | E₄*E₅ | E_{f} |
| 1049 | E₁ₐEᵢᵢᵢ | EᵥEᵥᵢ | E₄*E₅ | E_{f} |
| 1050 | E_{1b}Eᵢᵢᵢ | EᵥEᵥᵢ | E₄*E₅ | E_{f} |
| 1051 | E₁ₐEᵢᵢᵢE₄ | EᵥEᵥᵢ | E₄*E₅ | E_{f} |
| 1052 | E_{1b}EᵢᵢᵢE₄ | EᵥEᵥᵢ | E₄*E₅ | E_{f} |
| 1053 | E₁ₐEᵢᵥ | EᵥEᵥᵢ | E₄*E₅ | E_{f} |
| 1054 | E_{1b}Eᵢᵥ | EᵥEᵥᵢ | E₄*E₅ | E_{f} |
| 1055 | E₁ₐEᵢᵥE₄ | EᵥEᵥᵢ | E₄*E₅ | E_{f} |
| 1056 | E_{1b}EᵢᵥE₄ | EᵥEᵥᵢ | E₄*E₅ | E_{f} |
| 1057 | E₁ₐ | AlkLEᵥEᵥᵢ | | E_{f} |
| 1058 | E_{1b} | AlkLEᵥEᵥᵢ | | E_{f} |
| 1059 | E₁ₐEᵢ | AlkLEᵥEᵥᵢ | | E_{f} |
| 1060 | E_{1b}Eᵢ | AlkLEᵥEᵥᵢ | | E_{f} |
| 1061 | E₁ₐEᵢᵢ | AlkLEᵥEᵥᵢ | | E_{f} |
| 1062 | E_{1b}Eᵢᵢ | AlkLEᵥEᵥᵢ | | E_{f} |
| 1063 | E₁ₐEᵢᵢE_{iib} | AlkLEᵥEᵥᵢ | | E_{f} |
| 1064 | E_{1b}EᵢᵢE_{iib} | AlkLEᵥEᵥᵢ | | E_{f} |
| 1065 | E₁ₐE₄ | AlkLEᵥEᵥᵢ | | E_{f} |
| 1066 | E_{1b}E₄ | AlkLEᵥEᵥᵢ | | E_{f} |
| 1067 | E₁ₐEᵢE₄ | AlkLEᵥEᵥᵢ | | E_{f} |
| 1068 | E_{1b}EᵢE₄ | AlkLEᵥEᵥᵢ | | E_{f} |
| 1069 | E₁ₐEᵢᵢE₄ | AlkLEᵥEᵥᵢ | | E_{f} |
| 1070 | E_{1b}EᵢᵢE₄ | AlkLEᵥEᵥᵢ | | E_{f} |
| 1071 | E₁ₐEᵢᵢE_{iib}E₄ | AlkLEᵥEᵥᵢ | | E_{f} |
| 1072 | E_{1b}EᵢᵢE_{iib}E₄ | AlkLEᵥEᵥᵢ | | E_{f} |
| 1073 | E₁ₐEᵢᵢᵢ | AlkLEᵥEᵥᵢ | | E_{f} |
| 1074 | E_{1b}Eᵢᵢᵢ | AlkLEᵥEᵥᵢ | | E_{f} |
| 1075 | E₁ₐEᵢᵢᵢE₄ | AlkLEᵥEᵥᵢ | | E_{f} |
| 1076 | E_{1b}EᵢᵢᵢE₄ | AlkLEᵥEᵥᵢ | | E_{f} |
| 1077 | E₁ₐEᵢᵥ | AlkLEᵥEᵥᵢ | | E_{f} |
| 1078 | E_{1b}Eᵢᵥ | AlkLEᵥEᵥᵢ | | E_{f} |
| 1079 | E₁ₐEᵢᵥE₄ | AlkLEᵥEᵥᵢ | | E_{f} |
| 1080 | E_{1b}EᵢᵥE₄ | AlkLEᵥEᵥᵢ | | E_{f} |
| 1081 | E₁ₐ | AlkLEᵥEᵥᵢ | E₄* | E_{f} |
| 1082 | E_{1b} | AlkLEᵥEᵥᵢ | E₄* | E_{f} |
| 1083 | E₁ₐEᵢ | AlkLEᵥEᵥᵢ | E₄* | E_{f} |
| 1084 | E_{1b}Eᵢ | AlkLEᵥEᵥᵢ | E₄* | E_{f} |
| 1085 | E₁ₐEᵢᵢ | AlkLEᵥEᵥᵢ | E₄* | E_{f} |
| 1086 | E_{1b}Eᵢᵢ | AlkLEᵥEᵥᵢ | E₄* | E_{f} |
| 1087 | E₁ₐEᵢᵢE_{iib} | AlkLEᵥEᵥᵢ | E₄* | E_{f} |
| 1088 | E_{1b}EᵢᵢE_{iib} | AlkLEᵥEᵥᵢ | E₄* | E_{f} |
| 1089 | E₁ₐE₄ | AlkLEᵥEᵥᵢ | E₄* | E_{f} |
| 1090 | E_{1b}E₄ | AlkLEᵥEᵥᵢ | E₄* | E_{f} |
| 1091 | E₁ₐEᵢE₄ | AlkLEᵥEᵥᵢ | E₄* | E_{f} |
| 1092 | E_{1b}EᵢE₄ | AlkLEᵥEᵥᵢ | E₄* | E_{f} |
| 1093 | E₁ₐEᵢᵢE₄ | AlkLEᵥEᵥᵢ | E₄* | E_{f} |
| 1094 | E_{1b}EᵢᵢE₄ | AlkLEᵥEᵥᵢ | E₄* | E_{f} |
| 1095 | E₁ₐEᵢᵢE_{iib}E₄ | AlkLEᵥEᵥᵢ | E₄* | E_{f} |
| 1096 | E_{1b}EᵢᵢE_{iib}E₄ | AlkLEᵥEᵥᵢ | E₄* | E_{f} |
| 1097 | E₁ₐEᵢᵢᵢ | AlkLEᵥEᵥᵢ | E₄* | E_{f} |
| 1098 | E_{1b}Eᵢᵢᵢ | AlkLEᵥEᵥᵢ | E₄* | E_{f} |
| 1099 | E₁ₐEᵢᵢᵢE₄ | AlkLEᵥEᵥᵢ | E₄* | E_{f} |
| 1100 | E_{1b}EᵢᵢᵢE₄ | AlkLEᵥEᵥᵢ | E₄* | E_{f} |
| 1101 | E₁ₐEᵢᵥ | AlkLEᵥEᵥᵢ | E₄* | E_{f} |
| 1102 | E_{1b}Eᵢᵥ | AlkLEᵥEᵥᵢ | E₄* | E_{f} |
| 1103 | E₁ₐEᵢᵥE₄ | AlkLEᵥEᵥᵢ | E₄* | E_{f} |
| 1104 | E_{1b}EᵢᵥE₄ | AlkLEᵥEᵥᵢ | E₄* | E_{f} |
| 1105 | E₁ₐ | AlkLEᵥEᵥᵢ | E₅ | E_{f} |
| 1106 | E_{1b} | AlkLEᵥEᵥᵢ | E₅ | E_{f} |
| 1107 | E₁ₐEᵢ | AlkLEᵥEᵥᵢ | E₅ | E_{f} |
| 1108 | E_{1b}Eᵢ | AlkLEᵥEᵥᵢ | E₅ | E_{f} |
| 1109 | E₁ₐEᵢᵢ | AlkLEᵥEᵥᵢ | E₅ | E_{f} |
| 1110 | E_{1b}Eᵢᵢ | AlkLEᵥEᵥᵢ | E₅ | E_{f} |
| 1111 | E₁ₐEᵢᵢE_{iib} | AlkLEᵥEᵥᵢ | E₅ | E_{f} |
| 1112 | E_{1b}EᵢᵢE_{iib} | AlkLEᵥEᵥᵢ | E₅ | E_{f} |
| 1113 | E₁ₐE₄ | AlkLEᵥEᵥᵢ | E₅ | E_{f} |
| 1114 | E_{1b}E₄ | AlkLEᵥEᵥᵢ | E₅ | E_{f} |
| 1115 | E₁ₐEᵢE₄ | AlkLEᵥEᵥᵢ | E₅ | E_{f} |
| 1116 | E_{1b}EᵢE₄ | AlkLEᵥEᵥᵢ | E₅ | E_{f} |
| 1117 | E₁ₐEᵢᵢE₄ | AlkLEᵥEᵥᵢ | E₅ | E_{f} |
| 1118 | E_{1b}EᵢᵢE₄ | AlkLEᵥEᵥᵢ | E₅ | E_{f} |
| 1119 | E₁ₐEᵢᵢE_{iib}E₄ | AlkLEᵥEᵥᵢ | E₅ | E_{f} |
| 1120 | E_{1b}EᵢᵢE_{iib}E₄ | AlkLEᵥEᵥᵢ | E₅ | E_{f} |
| 1121 | E₁ₐEᵢᵢᵢ | AlkLEᵥEᵥᵢ | E₅ | E_{f} |
| 1122 | E_{1b}Eᵢᵢᵢ | AlkLEᵥEᵥᵢ | E₅ | E_{f} |
| 1123 | E₁ₐEᵢᵢᵢE₄ | AlkLEᵥEᵥᵢ | E₅ | E_{f} |
| 1124 | E_{1b}EᵢᵢᵢE₄ | AlkLEᵥEᵥᵢ | E₅ | E_{f} |
| 1125 | E₁ₐEᵢᵥ | AlkLEᵥEᵥᵢ | E₅ | E_{f} |
| 1126 | E_{1b}Eᵢᵥ | AlkLEᵥEᵥᵢ | E₅ | E_{f} |
| 1127 | E₁ₐEᵢᵥE₄ | AlkLEᵥEᵥᵢ | E₅ | E_{f} |
| 1128 | E_{1b}EᵢᵥE₄ | AlkLEᵥEᵥᵢ | E₅ | E_{f} |
| 1129 | E₁ₐ | AlkLEᵥEᵥᵢ | E₄*E₅ | E_{f} |
| 1130 | E_{1b} | AlkLEᵥEᵥᵢ | E₄*E₅ | E_{f} |
| 1131 | E₁ₐEᵢ | AlkLEᵥEᵥᵢ | E₄*E₅ | E_{f} |
| 1132 | E_{1b}Eᵢ | AlkLEᵥEᵥᵢ | E₄*E₅ | E_{f} |
| 1133 | E₁ₐEᵢᵢ | AlkLEᵥEᵥᵢ | E₄*E₅ | E_{f} |
| 1134 | E_{1b}Eᵢᵢ | AlkLEᵥEᵥᵢ | E₄*E₅ | E_{f} |
| 1135 | E₁ₐEᵢᵢE_{iib} | AlkLEᵥEᵥᵢ | E₄*E₅ | E_{f} |
| 1136 | E_{1b}EᵢᵢE_{iib} | AlkLEᵥEᵥᵢ | E₄*E₅ | E_{f} |
| 1137 | E₁ₐE₄ | AlkLEᵥEᵥᵢ | E₄*E₅ | E_{f} |
| 1138 | E_{1b}E₄ | AlkLEᵥEᵥᵢ | E₄*E₅ | E_{f} |
| 1139 | E₁ₐEᵢE₄ | AlkLEᵥEᵥᵢ | E₄*E₅ | E_{f} |
| 1140 | E_{1b}EᵢE₄ | AlkLEᵥEᵥᵢ | E₄*E₅ | E_{f} |
| 1141 | E₁ₐEᵢᵢE₄ | AlkLEᵥEᵥᵢ | E₄*E₅ | E_{f} |
| 1142 | E_{1b}EᵢᵢE₄ | AlkLEᵥEᵥᵢ | E₄*E₅ | E_{f} |
| 1143 | E₁ₐEᵢᵢE_{iib}E₄ | AlkLEᵥEᵥᵢ | E₄*E₅ | E_{f} |
| 1144 | E_{1b}EᵢᵢE_{iib}E₄ | AlkLEᵥEᵥᵢ | E₄*E₅ | E_{f} |
| 1145 | E₁ₐEᵢᵢᵢ | AlkLEᵥEᵥᵢ | E₄*E₅ | E_{f} |
| 1146 | E_{1b}Eᵢᵢᵢ | AlkLEᵥEᵥᵢ | E₄*E₅ | E_{f} |
| 1147 | E₁ₐEᵢᵢᵢE₄ | AlkLEᵥEᵥᵢ | E₄*E₅ | E_{f} |
| 1148 | E_{1b}EᵢᵢᵢE₄ | AlkLEᵥEᵥᵢ | E₄*E₅ | E_{f} |
| 1149 | E₁ₐEᵢᵥ | AlkLEᵥEᵥᵢ | E₄*E₅ | E_{f} |
| 1150 | E_{1b}Eᵢᵥ | AlkLEᵥEᵥᵢ | E₄*E₅ | E_{f} |
| 1151 | E₁ₐEᵢᵥE₄ | AlkLEᵥEᵥᵢ | E₄*E₅ | E_{f} |
| 1152 | E_{1b}EᵢᵥE₄ | AlkLEᵥEᵥᵢ | E₄*E₅ | E_{f} |
| 1153 | E₁ₐ | Eᵥᵢᵢ | | |
| 1154 | E_{1b} | Eᵥᵢᵢ | | |
| 1155 | E₁ₐEᵢ | Eᵥᵢᵢ | | |
| 1156 | E_{1b}Eᵢ | Eᵥᵢᵢ | | |
| 1157 | E₁ₐEᵢᵢ | Eᵥᵢᵢ | | |
| 1158 | E_{1b}Eᵢᵢ | Eᵥᵢᵢ | | |
| 1159 | E₁ₐEᵢᵢE_{iib} | Eᵥᵢᵢ | | |
| 1160 | E_{1b}EᵢᵢE_{iib} | Eᵥᵢᵢ | | |
| 1161 | E₁ₐE₄ | Eᵥᵢᵢ | | |
| 1162 | E_{1b}E₄ | Eᵥᵢᵢ | | |
| 1163 | E₁ₐEᵢE₄ | Eᵥᵢᵢ | | |
| 1164 | E_{1b}EᵢE₄ | Eᵥᵢᵢ | | |
| 1165 | E₁ₐEᵢᵢE₄ | Eᵥᵢᵢ | | |
| 1166 | E_{1b}EᵢᵢE₄ | Eᵥᵢᵢ | | |
| 1167 | E₁ₐEᵢᵢE_{iib}E₄ | Eᵥᵢᵢ | | |
| 1168 | E_{1b}EᵢᵢE_{iib}E₄ | Eᵥᵢᵢ | | |
| 1169 | E₁ₐEᵢᵢᵢ | Eᵥᵢᵢ | | |
| 1170 | E_{1b}Eᵢᵢᵢ | Eᵥᵢᵢ | | |
| 1171 | E₁ₐEᵢᵢᵢE₄ | Eᵥᵢᵢ | | |
| 1172 | E_{1b}EᵢᵢᵢE₄ | Eᵥᵢᵢ | | |
| 1173 | E₁ₐEᵢᵥ | Eᵥᵢᵢ | | |
| 1174 | E_{1b}Eᵢᵥ | Eᵥᵢᵢ | | |
| 1175 | E₁ₐEᵢᵥE₄ | Eᵥᵢᵢ | | |
| 1176 | E_{1b}EᵢᵥE₄ | Eᵥᵢᵢ | | |
| 1177 | E₁ₐ | Eᵥᵢᵢ | E₄* | |
| 1178 | E_{1b} | Eᵥᵢᵢ | E₄* | |
| 1179 | E₁ₐEᵢ | Eᵥᵢᵢ | E₄* | |
| 1180 | E_{1b}Eᵢ | Eᵥᵢᵢ | E₄* | |
| 1181 | E₁ₐEᵢᵢ | Eᵥᵢᵢ | E₄* | |
| 1182 | E_{1b}Eᵢᵢ | Eᵥᵢᵢ | E₄* | |
| 1183 | E₁ₐEᵢᵢE_{iib} | Eᵥᵢᵢ | E₄* | |
| 1184 | E_{1b}EᵢᵢE_{iib} | Eᵥᵢᵢ | E₄* | |
| 1185 | E₁ₐE₄ | Eᵥᵢᵢ | E₄* | |
| 1186 | E_{1b}E₄ | Eᵥᵢᵢ | E₄* | |
| 1187 | E₁ₐEᵢE₄ | Eᵥᵢᵢ | E₄* | |
| 1188 | E_{1b}EᵢE₄ | Eᵥᵢᵢ | E₄* | |
| 1189 | E₁ₐEᵢᵢE₄ | Eᵥᵢᵢ | E₄* | |
| 1190 | E_{1b}EᵢᵢE₄ | Eᵥᵢᵢ | E₄* | |
| 1191 | E₁ₐEᵢᵢE_{iib}E₄ | Eᵥᵢᵢ | E₄* | |
| 1192 | E_{1b}EᵢᵢE_{iib}E₄ | Eᵥᵢᵢ | E₄* | |
| 1193 | E₁ₐEᵢᵢᵢ | Eᵥᵢᵢ | E₄* | |
| 1194 | E_{1b}Eᵢᵢᵢ | Eᵥᵢᵢ | E₄* | |
| 1195 | E₁ₐEᵢᵢᵢE₄ | Eᵥᵢᵢ | E₄* | |
| 1196 | E_{1b}EᵢᵢᵢE₄ | Eᵥᵢᵢ | E₄* | |
| 1197 | E₁ₐEᵢᵥ | Eᵥᵢᵢ | E₄* | |
| 1198 | E_{1b}Eᵢᵥ | Eᵥᵢᵢ | E₄* | |
| 1199 | E₁ₐEᵢᵥE₄ | Eᵥᵢᵢ | E₄* | |
| 1200 | E_{1b}EᵢᵥE₄ | Eᵥᵢᵢ | E₄* | |
| 1201 | E₁ₐ | Eᵥᵢᵢ | E₅ | |
| 1202 | E_{1b} | Eᵥᵢᵢ | E₅ | |
| 1203 | E₁ₐEᵢ | Eᵥᵢᵢ | E₅ | |
| 1204 | E_{1b}Eᵢ | Eᵥᵢᵢ | E₅ | |
| 1205 | E₁ₐEᵢᵢ | Eᵥᵢᵢ | E₅ | |
| 1206 | E_{1b}Eᵢᵢ | Eᵥᵢᵢ | E₅ | |
| 1207 | E₁ₐEᵢᵢE_{iib} | Eᵥᵢᵢ | E₅ | |
| 1208 | E_{1b}EᵢᵢE_{iib} | Eᵥᵢᵢ | E₅ | |
| 1209 | E₁ₐE₄ | Eᵥᵢᵢ | E₅ | |
| 1210 | E_{1b}E₄ | Eᵥᵢᵢ | E₅ | |
| 1211 | E₁ₐEᵢE₄ | Eᵥᵢᵢ | E₅ | |
| 1212 | E_{1b}EᵢE₄ | Eᵥᵢᵢ | E₅ | |
| 1213 | E₁ₐEᵢᵢE₄ | Eᵥᵢᵢ | E₅ | |
| 1214 | E_{1b}EᵢᵢE₄ | Eᵥᵢᵢ | E₅ | |
| 1215 | E₁ₐEᵢᵢE_{iib}E₄ | Eᵥᵢᵢ | E₅ | |
| 1216 | E_{1b}EᵢᵢE_{iib}E₄ | Eᵥᵢᵢ | E₅ | |
| 1217 | E₁ₐEᵢᵢᵢ | Eᵥᵢᵢ | E₅ | |
| 1218 | E_{1b}Eᵢᵢᵢ | Eᵥᵢᵢ | E₅ | |
| 1219 | E₁ₐEᵢᵢᵢE₄ | Eᵥᵢᵢ | E₅ | |
| 1220 | E_{1b}EᵢᵢᵢE₄ | Eᵥᵢᵢ | E₅ | |
| 1221 | E₁ₐEᵢᵥ | Eᵥᵢᵢ | E₅ | |
| 1222 | E_{1b}Eᵢᵥ | Eᵥᵢᵢ | E₅ | |
| 1223 | E₁ₐEᵢᵥE₄ | Eᵥᵢᵢ | E₅ | |
| 1224 | E_{1b}EᵢᵥE₄ | Eᵥᵢᵢ | E₅ | |
| 1225 | E₁ₐ | Eᵥᵢᵢ | E₄*E₅ | |
| 1226 | E_{1b} | Eᵥᵢᵢ | E₄*E₅ | |
| 1227 | E₁ₐEᵢ | Eᵥᵢᵢ | E₄*E₅ | |
| 1228 | E_{1b}Eᵢ | Eᵥᵢᵢ | E₄*E₅ | |
| 1229 | E₁ₐEᵢᵢ | Eᵥᵢᵢ | E₄*E₅ | |
| 1230 | E_{1b}Eᵢᵢ | Eᵥᵢᵢ | E₄*E₅ | |
| 1231 | E₁ₐEᵢᵢE_{iib} | Eᵥᵢᵢ | E₄*E₅ | |
| 1232 | E_{1b}EᵢᵢE_{iib} | Eᵥᵢᵢ | E₄*E₅ | |
| 1233 | E₁ₐE₄ | Eᵥᵢᵢ | E₄*E₅ | |
| 1234 | E_{1b}E₄ | Eᵥᵢᵢ | E₄*E₅ | |
| 1235 | E₁ₐEᵢE₄ | Eᵥᵢᵢ | E₄*E₅ | |
| 1236 | E_{1b}EᵢE₄ | Eᵥᵢᵢ | E₄*E₅ | |
| 1237 | E₁ₐEᵢᵢE₄ | Eᵥᵢᵢ | E₄*E₅ | |
| 1238 | E_{1b}EᵢᵢE₄ | Eᵥᵢᵢ | E₄*E₅ | |
| 1239 | E₁ₐEᵢᵢE_{iib}E₄ | Eᵥᵢᵢ | E₄*E₅ | |
| 1240 | E_{1b}EᵢᵢE_{iib}E₄ | Eᵥᵢᵢ | E₄*E₅ | |
| 1241 | E₁ₐEᵢᵢᵢ | Eᵥᵢᵢ | E₄*E₅ | |
| 1242 | E_{1b}Eᵢᵢᵢ | Eᵥᵢᵢ | E₄*E₅ | |
| 1243 | E₁ₐEᵢᵢᵢE₄ | Eᵥᵢᵢ | E₄*E₅ | |
| 1244 | E_{1b}EᵢᵢᵢE₄ | Eᵥᵢᵢ | E₄*E₅ | |
| 1245 | E₁ₐEᵢᵥ | Eᵥᵢᵢ | E₄*E₅ | |
| 1246 | E_{1b}Eᵢᵥ | Eᵥᵢᵢ | E₄*E₅ | |
| 1247 | E₁ₐEᵢᵥE₄ | Eᵥᵢᵢ | E₄*E₅ | |
| 1248 | E_{1b}EᵢᵥE₄ | Eᵥᵢᵢ | E₄*E₅ | |
| 1249 | E₁ₐ | AlkLEᵥᵢᵢ | | |
| 1250 | E_{1b} | AlkLEᵥᵢᵢ | | |
| 1251 | E₁ₐEᵢ | AlkLEᵥᵢᵢ | | |
| 1252 | E_{1b}Eᵢ | AlkLEᵥᵢᵢ | | |
| 1253 | E₁ₐEᵢᵢ | AlkLEᵥᵢᵢ | | |
| 1254 | E_{1b}Eᵢᵢ | AlkLEᵥᵢᵢ | | |
| 1255 | E₁ₐEᵢᵢE_{iib} | AlkLEᵥᵢᵢ | | |
| 1256 | E_{1b}EᵢᵢE_{iib} | AlkLEᵥᵢᵢ | | |
| 1257 | E₁ₐE₄ | AlkLEᵥᵢᵢ | | |
| 1258 | E_{1b}E₄ | AlkLEᵥᵢᵢ | | |
| 1259 | E₁ₐEᵢE₄ | AlkLEᵥᵢᵢ | | |
| 1260 | E_{1b}EᵢE₄ | AlkLEᵥᵢᵢ | | |
| 1261 | E₁ₐEᵢᵢE₄ | AlkLEᵥᵢᵢ | | |
| 1262 | E_{1b}EᵢᵢE₄ | AlkLEᵥᵢᵢ | | |
| 1263 | E₁ₐEᵢᵢE_{iib}E₄ | AlkLEᵥᵢᵢ | | |
| 1264 | E_{1b}EᵢᵢE_{iib}E₄ | AlkLEᵥᵢᵢ | | |
| 1265 | E₁ₐEᵢᵢᵢ | AlkLEᵥᵢᵢ | | |
| 1266 | E_{1b}Eᵢᵢᵢ | AlkLEᵥᵢᵢ | | |
| 1267 | E₁ₐEᵢᵢᵢE₄ | AlkLEᵥᵢᵢ | | |
| 1268 | E_{1b}EᵢᵢᵢE₄ | AlkLEᵥᵢᵢ | | |
| 1269 | E₁ₐEᵢᵥ | AlkLEᵥᵢᵢ | | |
| 1270 | E_{1b}Eᵢᵥ | AlkLEᵥᵢᵢ | | |
| 1271 | E₁ₐEᵢᵥE₄ | AlkLEᵥᵢᵢ | | |
| 1272 | E_{1b}EᵢᵥE₄ | AlkLEᵥᵢᵢ | | |
| 1273 | E₁ₐ | AlkLEᵥᵢᵢ | E₄* | |
| 1274 | E_{1b} | AlkLEᵥᵢᵢ | E₄* | |
| 1275 | E₁ₐEᵢ | AlkLEᵥᵢᵢ | E₄* | |
| 1276 | E_{1b}Eᵢ | AlkLEᵥᵢᵢ | E₄* | |
| 1277 | E₁ₐEᵢᵢ | AlkLEᵥᵢᵢ | E₄* | |
| 1278 | E_{1b}Eᵢᵢ | AlkLEᵥᵢᵢ | E₄* | |
| 1279 | E₁ₐEᵢᵢE_{iib} | AlkLEᵥᵢᵢ | E₄* | |
| 1280 | E_{1b}EᵢᵢE_{iib} | AlkLEᵥᵢᵢ | E₄* | |
| 1281 | E₁ₐE₄ | AlkLEᵥᵢᵢ | E₄* | |
| 1282 | E_{1b}E₄ | AlkLEᵥᵢᵢ | E₄* | |
| 1283 | E₁ₐEᵢE₄ | AlkLEᵥᵢᵢ | E₄* | |
| 1284 | E_{1b}EᵢE₄ | AlkLEᵥᵢᵢ | E₄* | |
| 1285 | E₁ₐEᵢᵢE₄ | AlkLEᵥᵢᵢ | E₄* | |
| 1286 | E_{1b}EᵢᵢE₄ | AlkLEᵥᵢᵢ | E₄* | |
| 1287 | E₁ₐEᵢᵢE_{iib}E₄ | AlkLEᵥᵢᵢ | E₄* | |
| 1288 | E_{1b}EᵢᵢE_{iib}E₄ | AlkLEᵥᵢᵢ | E₄* | |
| 1289 | E₁ₐEᵢᵢᵢ | AlkLEᵥᵢᵢ | E₄* | |
| 1290 | E_{1b}Eᵢᵢᵢ | AlkLEᵥᵢᵢ | E₄* | |
| 1291 | E₁ₐEᵢᵢᵢE₄ | AlkLEᵥᵢᵢ | E₄* | |
| 1292 | E_{1b}EᵢᵢᵢE₄ | AlkLEᵥᵢᵢ | E₄* | |
| 1293 | E₁ₐEᵢᵥ | AlkLEᵥᵢᵢ | E₄* | |
| 1294 | E_{1b}Eᵢᵥ | AlkLEᵥᵢᵢ | E₄* | |
| 1295 | E₁ₐEᵢᵥE₄ | AlkLEᵥᵢᵢ | E₄* | |
| 1296 | E_{1b}EᵢᵥE₄ | AlkLEᵥᵢᵢ | E₄* | |
| 1297 | E₁ₐ | AlkLEᵥᵢᵢ | E₅ | |
| 1298 | E_{1b} | AlkLEᵥᵢᵢ | E₅ | |
| 1299 | E₁ₐEᵢ | AlkLEᵥᵢᵢ | E₅ | |
| 1300 | E_{1b}Eᵢ | AlkLEᵥᵢᵢ | E₅ | |
| 1301 | E₁ₐEᵢᵢ | AlkLEᵥᵢᵢ | E₅ | |
| 1302 | E_{1b}Eᵢᵢ | AlkLEᵥᵢᵢ | E₅ | |
| 1303 | E₁ₐEᵢᵢE_{iib} | AlkLEᵥᵢᵢ | E₅ | |
| 1304 | E_{1b}EᵢᵢE_{iib} | AlkLEᵥᵢᵢ | E₅ | |
| 1305 | E₁ₐE₄ | AlkLEᵥᵢᵢ | E₅ | |
| 1306 | E_{1b}E₄ | AlkLEᵥᵢᵢ | E₅ | |
| 1307 | E₁ₐEᵢE₄ | AlkLEᵥᵢᵢ | E₅ | |
| 1308 | E_{1b}EᵢE₄ | AlkLEᵥᵢᵢ | E₅ | |
| 1309 | E₁ₐEᵢᵢE₄ | AlkLEᵥᵢᵢ | E₅ | |
| 1310 | E_{1b}EᵢᵢE₄ | AlkLEᵥᵢᵢ | E₅ | |
| 1311 | E₁ₐEᵢᵢE_{iib}E₄ | AlkLEᵥᵢᵢ | E₅ | |
| 1312 | E_{1b}EᵢᵢE_{iib}E₄ | AlkLEᵥᵢᵢ | E₅ | |
| 1313 | E₁ₐEᵢᵢᵢ | AlkLEᵥᵢᵢ | E₅ | |
| 1314 | E_{1b}Eᵢᵢᵢ | AlkLEᵥᵢᵢ | E₅ | |
| 1315 | E₁ₐEᵢᵢᵢE₄ | AlkLEᵥᵢᵢ | E₅ | |
| 1316 | E_{1b}EᵢᵢᵢE₄ | AlkLEᵥᵢᵢ | E₅ | |
| 1317 | E₁ₐEᵢᵥ | AlkLEᵥᵢᵢ | E₅ | |
| 1318 | E_{1b}Eᵢᵥ | AlkLEᵥᵢᵢ | E₅ | |
| 1319 | E₁ₐEᵢᵥE₄ | AlkLEᵥᵢᵢ | E₅ | |
| 1320 | E_{1b}EᵢᵥE₄ | AlkLEᵥᵢᵢ | E₅ | |
| 1321 | E₁ₐ | AlkLEᵥᵢᵢ | E₄*E₅ | |
| 1322 | E_{1b} | AlkLEᵥᵢᵢ | E₄*E₅ | |
| 1323 | E₁ₐEᵢ | AlkLEᵥᵢᵢ | E₄*E₅ | |
| 1324 | E_{1b}Eᵢ | AlkLEᵥᵢᵢ | E₄*E₅ | |
| 1325 | E₁ₐEᵢᵢ | AlkLEᵥᵢᵢ | E₄*E₅ | |
| 1326 | E_{1b}Eᵢᵢ | AlkLEᵥᵢᵢ | E₄*E₅ | |
| 1327 | E₁ₐEᵢᵢE_{iib} | AlkLEᵥᵢᵢ | E₄*E₅ | |
| 1328 | E_{1b}EᵢᵢE_{iib} | AlkLEᵥᵢᵢ | E₄*E₅ | |
| 1329 | E₁ₐE₄ | AlkLEᵥᵢᵢ | E₄*E₅ | |
| 1330 | E_{1b}E₄ | AlkLEᵥᵢᵢ | E₄*E₅ | |
| 1331 | E₁ₐEᵢE₄ | AlkLEᵥᵢᵢ | E₄*E₅ | |
| 1332 | E_{1b}EᵢE₄ | AlkLEᵥᵢᵢ | E₄*E₅ | |
| 1333 | E₁ₐEᵢᵢE₄ | AlkLEᵥᵢᵢ | E₄*E₅ | |
| 1334 | E_{1b}EᵢᵢE₄ | AlkLEᵥᵢᵢ | E₄*E₅ | |
| 1335 | E₁ₐEᵢᵢE_{iib}E₄ | AlkLEᵥᵢᵢ | E₄*E₅ | |
| 1336 | E_{1b}EᵢᵢE_{iib}E₄ | AlkLEᵥᵢᵢ | E₄*E₅ | |
| 1337 | E₁ₐEᵢᵢᵢ | AlkLEᵥᵢᵢ | E₄*E₅ | |
| 1338 | E_{1b}Eᵢᵢᵢ | AlkLEᵥᵢᵢ | E₄*E₅ | |
| 1339 | E₁ₐEᵢᵢᵢE₄ | AlkLEᵥᵢᵢ | E₄*E₅ | |
| 1340 | E_{1b}EᵢᵢᵢE₄ | AlkLEᵥᵢᵢ | E₄*E₅ | |
| 1341 | E₁ₐEᵢᵥ | AlkLEᵥᵢᵢ | E₄*E₅ | |
| 1342 | E_{1b}Eᵢᵥ | AlkLEᵥᵢᵢ | E₄*E₅ | |
| 1343 | E₁ₐEᵢᵥE₄ | AlkLEᵥᵢᵢ | E₄*E₅ | |
| 1344 | E_{1b}EᵢᵥE₄ | AlkLEᵥᵢᵢ | E₄*E₅ | |
| 1345 | E₁ₐ | Eᵥᵢᵢ | | Eₐ |
| 1346 | E_{1b} | Eᵥᵢᵢ | | Eₐ |
| 1347 | E₁ₐEᵢ | Eᵥᵢᵢ | | Eₐ |
| 1348 | E_{1b}Eᵢ | Eᵥᵢᵢ | | Eₐ |
| 1349 | E₁ₐEᵢᵢ | Eᵥᵢᵢ | | Eₐ |
| 1350 | E_{1b}Eᵢᵢ | Eᵥᵢᵢ | | Eₐ |
| 1351 | E₁ₐEᵢᵢE_{iib} | Eᵥᵢᵢ | | Eₐ |
| 1352 | E_{1b}EᵢᵢE_{iib} | Eᵥᵢᵢ | | Eₐ |
| 1353 | E₁ₐE₄ | Eᵥᵢᵢ | | Eₐ |
| 1354 | E_{1b}E₄ | Eᵥᵢᵢ | | Eₐ |
| 1355 | E₁ₐEᵢE₄ | Eᵥᵢᵢ | | Eₐ |
| 1356 | E_{1b}EᵢE₄ | Eᵥᵢᵢ | | Eₐ |
| 1357 | E₁ₐEᵢᵢE₄ | Eᵥᵢᵢ | | Eₐ |
| 1358 | E_{1b}EᵢᵢE₄ | Eᵥᵢᵢ | | Eₐ |
| 1359 | E₁ₐEᵢᵢE_{iib}E₄ | Eᵥᵢᵢ | | Eₐ |
| 1360 | E_{1b}EᵢᵢE_{iib}E₄ | Eᵥᵢᵢ | | Eₐ |
| 1361 | E₁ₐEᵢᵢᵢ | Eᵥᵢᵢ | | Eₐ |
| 1362 | E_{1b}Eᵢᵢᵢ | Eᵥᵢᵢ | | Eₐ |
| 1363 | E₁ₐEᵢᵢᵢE₄ | Eᵥᵢᵢ | | Eₐ |
| 1364 | E_{1b}EᵢᵢᵢE₄ | Eᵥᵢᵢ | | Eₐ |
| 1365 | E₁ₐEᵢᵥ | Eᵥᵢᵢ | | Eₐ |
| 1366 | E_{1b}Eᵢᵥ | Eᵥᵢᵢ | | Eₐ |
| 1367 | E₁ₐEᵢᵥE₄ | Eᵥᵢᵢ | | Eₐ |
| 1368 | E_{1b}EᵢᵥE₄ | Eᵥᵢᵢ | | Eₐ |
| 1369 | E₁ₐ | Eᵥᵢᵢ | E₄* | Eₐ |
| 1370 | E_{1b} | Eᵥᵢᵢ | E₄* | Eₐ |
| 1371 | E₁ₐEᵢ | Eᵥᵢᵢ | E₄* | Eₐ |
| 1372 | E_{1b}Eᵢ | Eᵥᵢᵢ | E₄* | Eₐ |
| 1373 | E₁ₐEᵢᵢ | Eᵥᵢᵢ | E₄* | Eₐ |
| 1374 | E_{1b}Eᵢᵢ | Eᵥᵢᵢ | E₄* | Eₐ |
| 1375 | E₁ₐEᵢᵢE_{iib} | Eᵥᵢᵢ | E₄* | Eₐ |
| 1376 | E_{1b}EᵢᵢE_{iib} | Eᵥᵢᵢ | E₄* | Eₐ |
| 1377 | E₁ₐE₄ | Eᵥᵢᵢ | E₄* | Eₐ |
| 1378 | E_{1b}E₄ | Eᵥᵢᵢ | E₄* | Eₐ |
| 1379 | E₁ₐEᵢE₄ | Eᵥᵢᵢ | E₄* | Eₐ |
| 1380 | E_{1b}EᵢE₄ | Eᵥᵢᵢ | E₄* | Eₐ |
| 1381 | E₁ₐEᵢᵢE₄ | Eᵥᵢᵢ | E₄* | Eₐ |
| 1382 | E_{1b}EᵢᵢE₄ | Eᵥᵢᵢ | E₄* | Eₐ |
| 1383 | E₁ₐEᵢᵢE_{iib}E₄ | Eᵥᵢᵢ | E₄* | Eₐ |
| 1384 | E_{1b}EᵢᵢE_{iib}E₄ | Eᵥᵢᵢ | E₄* | Eₐ |
| 1385 | E₁ₐEᵢᵢᵢ | Eᵥᵢᵢ | E₄* | Eₐ |
| 1386 | E_{1b}Eᵢᵢᵢ | Eᵥᵢᵢ | E₄* | Eₐ |
| 1387 | E₁ₐEᵢᵢᵢE₄ | Eᵥᵢᵢ | E₄* | Eₐ |
| 1388 | E_{1b}EᵢᵢᵢE₄ | Eᵥᵢᵢ | E₄* | Eₐ |
| 1389 | E₁ₐEᵢᵥ | Eᵥᵢᵢ | E₄* | Eₐ |
| 1390 | E_{1b}Eᵢᵥ | Eᵥᵢᵢ | E₄* | Eₐ |
| 1391 | E₁ₐEᵢᵥE₄ | Eᵥᵢᵢ | E₄* | Eₐ |
| 1392 | E_{1b}EᵢᵥE₄ | Eᵥᵢᵢ | E₄* | Eₐ |
| 1393 | E₁ₐ | Eᵥᵢᵢ | E₅ | Eₐ |
| 1394 | E_{1b} | Eᵥᵢᵢ | E₅ | Eₐ |
| 1395 | E₁ₐEᵢ | Eᵥᵢᵢ | E₅ | Eₐ |
| 1396 | E_{1b}Eᵢ | Eᵥᵢᵢ | E₅ | Eₐ |
| 1397 | E₁ₐEᵢᵢ | Eᵥᵢᵢ | E₅ | Eₐ |
| 1398 | E_{1b}Eᵢᵢ | Eᵥᵢᵢ | E₅ | Eₐ |
| 1399 | E₁ₐEᵢᵢE_{iib} | Eᵥᵢᵢ | E₅ | Eₐ |
| 1400 | E_{1b}EᵢᵢE_{iib} | Eᵥᵢᵢ | E₅ | Eₐ |
| 1401 | E₁ₐE₄ | Eᵥᵢᵢ | E₅ | Eₐ |
| 1402 | E_{1b}E₄ | Eᵥᵢᵢ | E₅ | Eₐ |
| 1403 | E₁ₐEᵢE₄ | Eᵥᵢᵢ | E₅ | Eₐ |
| 1404 | E_{1b}EᵢE₄ | Eᵥᵢᵢ | E₅ | Eₐ |
| 1405 | E₁ₐEᵢᵢE₄ | Eᵥᵢᵢ | E₅ | Eₐ |
| 1406 | E_{1b}EᵢᵢE₄ | Eᵥᵢᵢ | E₅ | Eₐ |
| 1407 | E₁ₐEᵢᵢE_{iib}E₄ | Eᵥᵢᵢ | E₅ | Eₐ |
| 1408 | E_{1b}EᵢᵢE_{iib}E₄ | Eᵥᵢᵢ | E₅ | Eₐ |
| 1409 | E₁ₐEᵢᵢᵢ | Eᵥᵢᵢ | E₅ | Eₐ |
| 1410 | E_{1b}Eᵢᵢᵢ | Eᵥᵢᵢ | E₅ | Eₐ |
| 1411 | E₁ₐEᵢᵢᵢE₄ | Eᵥᵢᵢ | E₅ | Eₐ |
| 1412 | E_{1b}EᵢᵢᵢE₄ | Eᵥᵢᵢ | E₅ | Eₐ |
| 1413 | E₁ₐEᵢᵥ | Eᵥᵢᵢ | E₅ | Eₐ |
| 1414 | E_{1b}Eᵢᵥ | Eᵥᵢᵢ | E₅ | Eₐ |
| 1415 | E₁ₐEᵢᵥE₄ | Eᵥᵢᵢ | E₅ | Eₐ |
| 1416 | E_{1b}EᵢᵥE₄ | Eᵥᵢᵢ | E₅ | Eₐ |
| 1417 | E₁ₐ | Eᵥᵢᵢ | E₄*E₅ | Eₐ |
| 1418 | E_{1b} | Eᵥᵢᵢ | E₄*E₅ | Eₐ |
| 1419 | E₁ₐEᵢ | Eᵥᵢᵢ | E₄*E₅ | Eₐ |
| 1420 | E_{1b}Eᵢ | Eᵥᵢᵢ | E₄*E₅ | Eₐ |
| 1421 | E₁ₐEᵢᵢ | Eᵥᵢᵢ | E₄*E₅ | Eₐ |
| 1422 | E_{1b}Eᵢᵢ | Eᵥᵢᵢ | E₄*E₅ | Eₐ |
| 1423 | E₁ₐEᵢᵢE_{iib} | Eᵥᵢᵢ | E₄*E₅ | Eₐ |
| 1424 | E_{1b}EᵢᵢE_{iib} | Eᵥᵢᵢ | E₄*E₅ | Eₐ |
| 1425 | E₁ₐE₄ | Eᵥᵢᵢ | E₄*E₅ | Eₐ |
| 1426 | E_{1b}E₄ | Eᵥᵢᵢ | E₄*E₅ | Eₐ |
| 1427 | E₁ₐEᵢE₄ | Eᵥᵢᵢ | E₄*E₅ | Eₐ |
| 1428 | E_{1b}EᵢE₄ | Eᵥᵢᵢ | E₄*E₅ | Eₐ |
| 1429 | E₁ₐEᵢᵢE₄ | Eᵥᵢᵢ | E₄*E₅ | Eₐ |
| 1430 | E_{1b}EᵢᵢE₄ | Eᵥᵢᵢ | E₄*E₅ | Eₐ |
| 1431 | E₁ₐEᵢᵢE_{iib}E₄ | Eᵥᵢᵢ | E₄*E₅ | Eₐ |
| 1432 | E_{1b}EᵢᵢE_{iib}E₄ | Eᵥᵢᵢ | E₄*E₅ | Eₐ |
| 1433 | E₁ₐEᵢᵢᵢ | Eᵥᵢᵢ | E₄*E₅ | Eₐ |
| 1434 | E_{1b}Eᵢᵢᵢ | Eᵥᵢᵢ | E₄*E₅ | Eₐ |
| 1435 | E₁ₐEᵢᵢᵢE₄ | Eᵥᵢᵢ | E₄*E₅ | Eₐ |
| 1436 | E_{1b}EᵢᵢᵢE₄ | Eᵥᵢᵢ | E₄*E₅ | Eₐ |
| 1437 | E₁ₐEᵢᵥ | Eᵥᵢᵢ | E₄*E₅ | Eₐ |
| 1438 | E_{1b}Eᵢᵥ | Eᵥᵢᵢ | E₄*E₅ | Eₐ |
| 1439 | E₁ₐEᵢᵥE₄ | Eᵥᵢᵢ | E₄*E₅ | Eₐ |
| 1440 | E_{1b}EᵢᵥE₄ | Eᵥᵢᵢ | E₄*E₅ | Eₐ |
| 1441 | E₁ₐ | AlkLEᵥᵢᵢ | | Eₐ |
| 1442 | E_{1b} | AlkLEᵥᵢᵢ | | Eₐ |
| 1443 | E₁ₐEᵢ | AlkLEᵥᵢᵢ | | Eₐ |
| 1444 | E_{1b}Eᵢ | AlkLEᵥᵢᵢ | | Eₐ |
| 1445 | E₁ₐEᵢᵢ | AlkLEᵥᵢᵢ | | Eₐ |
| 1446 | E_{1b}Eᵢᵢ | AlkLEᵥᵢᵢ | | Eₐ |
| 1447 | E₁ₐEᵢᵢE_{iib} | AlkLEᵥᵢᵢ | | Eₐ |
| 1448 | E_{1b}EᵢᵢE_{iib} | AlkLEᵥᵢᵢ | | Eₐ |
| 1449 | E₁ₐE₄ | AlkLEᵥᵢᵢ | | Eₐ |
| 1450 | E_{1b}E₄ | AlkLEᵥᵢᵢ | | Eₐ |
| 1451 | E₁ₐEᵢE₄ | AlkLEᵥᵢᵢ | | Eₐ |
| 1452 | E_{1b}EᵢE₄ | AlkLEᵥᵢᵢ | | Eₐ |
| 1453 | E₁ₐEᵢᵢE₄ | AlkLEᵥᵢᵢ | | Eₐ |
| 1454 | E_{1b}EᵢᵢE₄ | AlkLEᵥᵢᵢ | | Eₐ |
| 1455 | E₁ₐEᵢᵢE_{iib}E₄ | AlkLEᵥᵢᵢ | | Eₐ |
| 1456 | E_{1b}EᵢᵢE_{iib}E₄ | AlkLEᵥᵢᵢ | | Eₐ |
| 1457 | E₁ₐEᵢᵢᵢ | AlkLEᵥᵢᵢ | | Eₐ |
| 1458 | E_{1b}Eᵢᵢᵢ | AlkLEᵥᵢᵢ | | Eₐ |
| 1459 | E₁ₐEᵢᵢᵢE₄ | AlkLEᵥᵢᵢ | | Eₐ |
| 1460 | E_{1b}EᵢᵢᵢE₄ | AlkLEᵥᵢᵢ | | Eₐ |
| 1461 | E₁ₐEᵢᵥ | AlkLEᵥᵢᵢ | | Eₐ |
| 1462 | E_{1b}Eᵢᵥ | AlkLEᵥᵢᵢ | | Eₐ |
| 1463 | E₁ₐEᵢᵥE₄ | AlkLEᵥᵢᵢ | | Eₐ |
| 1464 | E_{1b}EᵢᵥE₄ | AlkLEᵥᵢᵢ | | Eₐ |
| 1465 | E₁ₐ | AlkLEᵥᵢᵢ | E₄* | Eₐ |
| 1466 | E_{1b} | AlkLEᵥᵢᵢ | E₄* | Eₐ |
| 1467 | E₁ₐEᵢ | AlkLEᵥᵢᵢ | E₄* | Eₐ |
| 1468 | E_{1b}Eᵢ | AlkLEᵥᵢᵢ | E₄* | Eₐ |
| 1469 | E₁ₐEᵢᵢ | AlkLEᵥᵢᵢ | E₄* | Eₐ |
| 1470 | E_{1b}Eᵢᵢ | AlkLEᵥᵢᵢ | E₄* | Eₐ |
| 1471 | E₁ₐEᵢᵢE_{iib} | AlkLEᵥᵢᵢ | E₄* | Eₐ |
| 1472 | E_{1b}EᵢᵢE_{iib} | AlkLEᵥᵢᵢ | E₄* | Eₐ |
| 1473 | E₁ₐE₄ | AlkLEᵥᵢᵢ | E₄* | Eₐ |
| 1474 | E_{1b}E₄ | AlkLEᵥᵢᵢ | E₄* | Eₐ |
| 1475 | E₁ₐEᵢE₄ | AlkLEᵥᵢᵢ | E₄* | Eₐ |
| 1476 | E_{1b}EᵢE₄ | AlkLEᵥᵢᵢ | E₄* | Eₐ |
| 1477 | E₁ₐEᵢᵢE₄ | AlkLEᵥᵢᵢ | E₄* | Eₐ |
| 1478 | E_{1b}EᵢᵢE₄ | AlkLEᵥᵢᵢ | E₄* | Eₐ |
| 1479 | E₁ₐEᵢᵢE_{iib}E₄ | AlkLEᵥᵢᵢ | E₄* | Eₐ |
| 1480 | E_{1b}EᵢᵢE_{iib}E₄ | AlkLEᵥᵢᵢ | E₄* | Eₐ |
| 1481 | E₁ₐEᵢᵢᵢ | AlkLEᵥᵢᵢ | E₄* | Eₐ |
| 1482 | E_{1b}Eᵢᵢᵢ | AlkLEᵥᵢᵢ | E₄* | Eₐ |
| 1483 | E₁ₐEᵢᵢᵢE₄ | AlkLEᵥᵢᵢ | E₄* | Eₐ |
| 1484 | E_{1b}EᵢᵢᵢE₄ | AlkLEᵥᵢᵢ | E₄* | Eₐ |
| 1485 | E₁ₐEᵢᵥ | AlkLEᵥᵢᵢ | E₄* | Eₐ |
| 1486 | E_{1b}Eᵢᵥ | AlkLEᵥᵢᵢ | E₄* | Eₐ |
| 1487 | E₁ₐEᵢᵥE₄ | AlkLEᵥᵢᵢ | E₄* | Eₐ |
| 1488 | E_{1b}EᵢᵥE₄ | AlkLEᵥᵢᵢ | E₄* | Eₐ |
| 1489 | E₁ₐ | AlkLEᵥᵢᵢ | E₅ | Eₐ |
| 1490 | E_{1b} | AlkLEᵥᵢᵢ | E₅ | Eₐ |
| 1491 | E₁ₐEᵢ | AlkLEᵥᵢᵢ | E₅ | Eₐ |
| 1492 | E_{1b}Eᵢ | AlkLEᵥᵢᵢ | E₅ | Eₐ |
| 1493 | E₁ₐEᵢᵢ | AlkLEᵥᵢᵢ | E₅ | Eₐ |
| 1494 | E_{1b}Eᵢᵢ | AlkLEᵥᵢᵢ | E₅ | Eₐ |
| 1495 | E₁ₐEᵢᵢE_{iib} | AlkLEᵥᵢᵢ | E₅ | Eₐ |
| 1496 | E_{1b}EᵢᵢE_{iib} | AlkLEᵥᵢᵢ | E₅ | Eₐ |
| 1497 | E₁ₐE₄ | AlkLEᵥᵢᵢ | E₅ | Eₐ |
| 1498 | E_{1b}E₄ | AlkLEᵥᵢᵢ | E₅ | Eₐ |
| 1499 | E₁ₐEᵢE₄ | AlkLEᵥᵢᵢ | E₅ | Eₐ |
| 1500 | E_{1b}EᵢE₄ | AlkLEᵥᵢᵢ | E₅ | Eₐ |
| 1501 | E₁ₐEᵢᵢE₄ | AlkLEᵥᵢᵢ | E₅ | Eₐ |
| 1502 | E_{1b}EᵢᵢE₄ | AlkLEᵥᵢᵢ | E₅ | Eₐ |
| 1503 | E₁ₐEᵢᵢE_{iib}E₄ | AlkLEᵥᵢᵢ | E₅ | Eₐ |
| 1504 | E_{1b}EᵢᵢE_{iib}E₄ | AlkLEᵥᵢᵢ | E₅ | Eₐ |
| 1505 | E₁ₐEᵢᵢᵢ | AlkLEᵥᵢᵢ | E₅ | Eₐ |
| 1506 | E_{1b}Eᵢᵢᵢ | AlkLEᵥᵢᵢ | E₅ | Eₐ |
| 1507 | E₁ₐEᵢᵢᵢE₄ | AlkLEᵥᵢᵢ | E₅ | Eₐ |
| 1508 | E_{1b}EᵢᵢᵢE₄ | AlkLEᵥᵢᵢ | E₅ | Eₐ |
| 1509 | E₁ₐEᵢᵥ | AlkLEᵥᵢᵢ | E₅ | Eₐ |
| 1510 | E_{1b}Eᵢᵥ | AlkLEᵥᵢᵢ | E₅ | Eₐ |
| 1511 | E₁ₐEᵢᵥE₄ | AlkLEᵥᵢᵢ | E₅ | Eₐ |
| 1512 | E_{1b}EᵢᵥE₄ | AlkLEᵥᵢᵢ | E₅ | Eₐ |
| 1513 | E₁ₐ | AlkLEᵥᵢᵢ | E₄*E₅ | Eₐ |
| 1514 | E_{1b} | AlkLEᵥᵢᵢ | E₄*E₅ | Eₐ |
| 1515 | E₁ₐEᵢ | AlkLEᵥᵢᵢ | E₄*E₅ | Eₐ |
| 1516 | E_{1b}Eᵢ | AlkLEᵥᵢᵢ | E₄*E₅ | Eₐ |
| 1517 | E₁ₐEᵢᵢ | AlkLEᵥᵢᵢ | E₄*E₅ | Eₐ |
| 1518 | E_{1b}Eᵢᵢ | AlkLEᵥᵢᵢ | E₄*E₅ | Eₐ |
| 1519 | E₁ₐEᵢᵢE_{iib} | AlkLEᵥᵢᵢ | E₄*E₅ | Eₐ |
| 1520 | E_{1b}EᵢᵢE_{iib} | AlkLEᵥᵢᵢ | E₄*E₅ | Eₐ |
| 1521 | E₁ₐE₄ | AlkLEᵥᵢᵢ | E₄*E₅ | Eₐ |
| 1522 | E_{1b}E₄ | AlkLEᵥᵢᵢ | E₄*E₅ | Eₐ |
| 1523 | E₁ₐEᵢE₄ | AlkLEᵥᵢᵢ | E₄*E₅ | Eₐ |
| 1524 | E_{1b}EᵢE₄ | AlkLEᵥᵢᵢ | E₄*E₅ | Eₐ |
| 1525 | E₁ₐEᵢᵢE₄ | AlkLEᵥᵢᵢ | E₄*E₅ | Eₐ |
| 1526 | E_{1b}EᵢᵢE₄ | AlkLEᵥᵢᵢ | E₄*E₅ | Eₐ |
| 1527 | E₁ₐEᵢᵢE_{iib}E₄ | AlkLEᵥᵢᵢ | E₄*E₅ | Eₐ |
| 1528 | E_{1b}EᵢᵢE_{iib}E₄ | AlkLEᵥᵢᵢ | E₄*E₅ | Eₐ |
| 1529 | E₁ₐEᵢᵢᵢ | AlkLEᵥᵢᵢ | E₄*E₅ | Eₐ |
| 1530 | E_{1b}Eᵢᵢᵢ | AlkLEᵥᵢᵢ | E₄*E₅ | Eₐ |
| 1531 | E₁ₐEᵢᵢᵢE₄ | AlkLEᵥᵢᵢ | E₄*E₅ | Eₐ |
| 1532 | E_{1b}EᵢᵢᵢE₄ | AlkLEᵥᵢᵢ | E₄*E₅ | Eₐ |
| 1533 | E₁ₐEᵢᵥ | AlkLEᵥᵢᵢ | E₄*E₅ | Eₐ |
| 1534 | E_{1b}Eᵢᵥ | AlkLEᵥᵢᵢ | E₄*E₅ | Eₐ |
| 1535 | E₁ₐEᵢᵥE₄ | AlkLEᵥᵢᵢ | E₄*E₅ | Eₐ |
| 1536 | E_{1b}EᵢᵥE₄ | AlkLEᵥᵢᵢ | E₄*E₅ | Eₐ |
| 1537 | E₁ₐ | Eᵥᵢᵢ | | E_{b} |
| 1538 | E_{1b} | Eᵥᵢᵢ | | E_{b} |
| 1539 | E₁ₐEᵢ | Eᵥᵢᵢ | | E_{b} |
| 1540 | E_{1b}Eᵢ | Eᵥᵢᵢ | | E_{b} |
| 1541 | E₁ₐEᵢᵢ | Eᵥᵢᵢ | | E_{b} |
| 1542 | E_{1b}Eᵢᵢ | Eᵥᵢᵢ | | E_{b} |
| 1543 | E₁ₐEᵢᵢE_{iib} | Eᵥᵢᵢ | | E_{b} |
| 1544 | E_{1b}EᵢᵢE_{iib} | Eᵥᵢᵢ | | E_{b} |
| 1545 | E₁ₐE₄ | Eᵥᵢᵢ | | E_{b} |
| 1546 | E_{1b}E₄ | Eᵥᵢᵢ | | E_{b} |
| 1547 | E₁ₐEᵢE₄ | Eᵥᵢᵢ | | E_{b} |
| 1548 | E_{1b}EᵢE₄ | Eᵥᵢᵢ | | E_{b} |
| 1549 | E₁ₐEᵢᵢE₄ | Eᵥᵢᵢ | | E_{b} |
| 1550 | E_{1b}EᵢᵢE₄ | Eᵥᵢᵢ | | E_{b} |
| 1551 | E₁ₐEᵢᵢE_{iib}E₄ | Eᵥᵢᵢ | | E_{b} |
| 1552 | E_{1b}EᵢᵢE_{iib}E₄ | Eᵥᵢᵢ | | E_{b} |
| 1553 | E₁ₐEᵢᵢᵢ | Eᵥᵢᵢ | | E_{b} |
| 1554 | E_{1b}Eᵢᵢᵢ | Eᵥᵢᵢ | | E_{b} |
| 1555 | E₁ₐEᵢᵢᵢE₄ | Eᵥᵢᵢ | | E_{b} |
| 1556 | E_{1b}EᵢᵢᵢE₄ | Eᵥᵢᵢ | | E_{b} |
| 1557 | E₁ₐEᵢᵥ | Eᵥᵢᵢ | | E_{b} |
| 1558 | E_{1b}Eᵢᵥ | Eᵥᵢᵢ | | E_{b} |
| 1559 | E₁ₐEᵢᵥE₄ | Eᵥᵢᵢ | | E_{b} |
| 1560 | E_{1b}EᵢᵥE₄ | Eᵥᵢᵢ | | E_{b} |
| 1561 | E₁ₐ | Eᵥᵢᵢ | E₄* | E_{b} |
| 1562 | E_{1b} | Eᵥᵢᵢ | E₄* | E_{b} |
| 1563 | E₁ₐEᵢ | Eᵥᵢᵢ | E₄* | E_{b} |
| 1564 | E_{1b}Eᵢ | Eᵥᵢᵢ | E₄* | E_{b} |
| 1565 | E₁ₐEᵢᵢ | Eᵥᵢᵢ | E₄* | E_{b} |
| 1566 | E_{1b}Eᵢᵢ | Eᵥᵢᵢ | E₄* | E_{b} |
| 1567 | E₁ₐEᵢᵢE_{iib} | Eᵥᵢᵢ | E₄* | E_{b} |
| 1568 | E_{1b}EᵢᵢE_{iib} | Eᵥᵢᵢ | E₄* | E_{b} |
| 1569 | E₁ₐE₄ | Eᵥᵢᵢ | E₄* | E_{b} |
| 1570 | E_{1b}E₄ | Eᵥᵢᵢ | E₄* | E_{b} |
| 1571 | E₁ₐEᵢE₄ | Eᵥᵢᵢ | E₄* | E_{b} |
| 1572 | E_{1b}EᵢE₄ | Eᵥᵢᵢ | E₄* | E_{b} |
| 1573 | E₁ₐEᵢᵢE₄ | Eᵥᵢᵢ | E₄* | E_{b} |
| 1574 | E_{1b}EᵢᵢE₄ | Eᵥᵢᵢ | E₄* | E_{b} |
| 1575 | E₁ₐEᵢᵢE_{iib}E₄ | Eᵥᵢᵢ | E₄* | E_{b} |
| 1576 | E_{1b}EᵢᵢE_{iib}E₄ | Eᵥᵢᵢ | E₄* | E_{b} |
| 1577 | E₁ₐEᵢᵢᵢ | Eᵥᵢᵢ | E₄* | E_{b} |
| 1578 | E_{1b}Eᵢᵢᵢ | Eᵥᵢᵢ | E₄* | E_{b} |
| 1579 | E₁ₐEᵢᵢᵢE₄ | Eᵥᵢᵢ | E₄* | E_{b} |
| 1580 | E_{1b}EᵢᵢᵢE₄ | Eᵥᵢᵢ | E₄* | E_{b} |
| 1581 | E₁ₐEᵢᵥ | Eᵥᵢᵢ | E₄* | E_{b} |
| 1582 | E_{1b}Eᵢᵥ | Eᵥᵢᵢ | E₄* | E_{b} |
| 1583 | E₁ₐEᵢᵥE₄ | Eᵥᵢᵢ | E₄* | E_{b} |
| 1584 | E_{1b}EᵢᵥE₄ | Eᵥᵢᵢ | E₄* | E_{b} |
| 1585 | E₁ₐ | Eᵥᵢᵢ | E₅ | E_{b} |
| 1586 | E_{1b} | Eᵥᵢᵢ | E₅ | E_{b} |
| 1587 | E₁ₐEᵢ | Eᵥᵢᵢ | E₅ | E_{b} |
| 1588 | E_{1b}Eᵢ | Eᵥᵢᵢ | E₅ | E_{b} |
| 1589 | E₁ₐEᵢᵢ | Eᵥᵢᵢ | E₅ | E_{b} |
| 1590 | E_{1b}Eᵢᵢ | Eᵥᵢᵢ | E₅ | E_{b} |
| 1591 | E₁ₐEᵢᵢE_{iib} | Eᵥᵢᵢ | E₅ | E_{b} |
| 1592 | E_{1b}EᵢᵢE_{iib} | Eᵥᵢᵢ | E₅ | E_{b} |
| 1593 | E₁ₐE₄ | Eᵥᵢᵢ | E₅ | E_{b} |
| 1594 | E_{1b}E₄ | Eᵥᵢᵢ | E₅ | E_{b} |
| 1595 | E₁ₐEᵢE₄ | Eᵥᵢᵢ | E₅ | E_{b} |
| 1596 | E_{1b}EᵢE₄ | Eᵥᵢᵢ | E₅ | E_{b} |
| 1597 | E₁ₐEᵢᵢE₄ | Eᵥᵢᵢ | E₅ | E_{b} |
| 1598 | E_{1b}EᵢᵢE₄ | Eᵥᵢᵢ | E₅ | E_{b} |
| 1599 | E₁ₐEᵢᵢE_{iib}E₄ | Eᵥᵢᵢ | E₅ | E_{b} |
| 1600 | E_{1b}EᵢᵢE_{iib}E₄ | Eᵥᵢᵢ | E₅ | E_{b} |
| 1601 | E₁ₐEᵢᵢᵢ | Eᵥᵢᵢ | E₅ | E_{b} |
| 1602 | E_{1b}Eᵢᵢᵢ | Eᵥᵢᵢ | E₅ | E_{b} |
| 1603 | E₁ₐEᵢᵢᵢE₄ | Eᵥᵢᵢ | E₅ | E_{b} |
| 1604 | E_{1b}EᵢᵢᵢE₄ | Eᵥᵢᵢ | E₅ | E_{b} |
| 1605 | E₁ₐEᵢᵥ | Eᵥᵢᵢ | E₅ | E_{b} |
| 1606 | E_{1b}Eᵢᵥ | Eᵥᵢᵢ | E₅ | E_{b} |
| 1607 | E₁ₐEᵢᵥE₄ | Eᵥᵢᵢ | E₅ | E_{b} |
| 1608 | E_{1b}EᵢᵥE₄ | Eᵥᵢᵢ | E₅ | E_{b} |
| 1609 | E₁ₐ | Eᵥᵢᵢ | E₄*E₅ | E_{b} |
| 1610 | E_{1b} | Eᵥᵢᵢ | E₄*E₅ | E_{b} |
| 1611 | E₁ₐEᵢ | Eᵥᵢᵢ | E₄*E₅ | E_{b} |
| 1612 | E_{1b}Eᵢ | Eᵥᵢᵢ | E₄*E₅ | E_{b} |
| 1613 | E₁ₐEᵢᵢ | Eᵥᵢᵢ | E₄*E₅ | E_{b} |
| 1614 | E_{1b}Eᵢᵢ | Eᵥᵢᵢ | E₄*E₅ | E_{b} |
| 1615 | E₁ₐEᵢᵢE_{iib} | Eᵥᵢᵢ | E₄*E₅ | E_{b} |
| 1616 | E_{1b}EᵢᵢE_{iib} | Eᵥᵢᵢ | E₄*E₅ | E_{b} |
| 1617 | E₁ₐE₄ | Eᵥᵢᵢ | E₄*E₅ | E_{b} |
| 1618 | E_{1b}E₄ | Eᵥᵢᵢ | E₄*E₅ | E_{b} |
| 1619 | E₁ₐEᵢE₄ | Eᵥᵢᵢ | E₄*E₅ | E_{b} |
| 1620 | E_{1b}EᵢE₄ | Eᵥᵢᵢ | E₄*E₅ | E_{b} |
| 1621 | E₁ₐEᵢᵢE₄ | Eᵥᵢᵢ | E₄*E₅ | E_{b} |
| 1622 | E_{1b}EᵢᵢE₄ | Eᵥᵢᵢ | E₄*E₅ | E_{b} |
| 1623 | E₁ₐEᵢᵢE_{iib}E₄ | Eᵥᵢᵢ | E₄*E₅ | E_{b} |
| 1624 | E_{1b}EᵢᵢE_{iib}E₄ | Eᵥᵢᵢ | E₄*E₅ | E_{b} |
| 1625 | E₁ₐEᵢᵢᵢ | Eᵥᵢᵢ | E₄*E₅ | E_{b} |
| 1626 | E_{1b}Eᵢᵢᵢ | Eᵥᵢᵢ | E₄*E₅ | E_{b} |
| 1627 | E₁ₐEᵢᵢᵢE₄ | Eᵥᵢᵢ | E₄*E₅ | E_{b} |
| 1628 | E_{1b}EᵢᵢᵢE₄ | Eᵥᵢᵢ | E₄*E₅ | E_{b} |
| 1629 | E₁ₐEᵢᵥ | Eᵥᵢᵢ | E₄*E₅ | E_{b} |
| 1630 | E_{1b}Eᵢᵥ | Eᵥᵢᵢ | E₄*E₅ | E_{b} |
| 1631 | E₁ₐEᵢᵥE₄ | Eᵥᵢᵢ | E₄*E₅ | E_{b} |
| 1632 | E_{1b}EᵢᵥE₄ | Eᵥᵢᵢ | E₄*E₅ | E_{b} |
| 1633 | E₁ₐ | AlkLEᵥᵢᵢ | | E_{b} |
| 1634 | E_{1b} | AlkLEᵥᵢᵢ | | E_{b} |
| 1635 | E₁ₐEᵢ | AlkLEᵥᵢᵢ | | E_{b} |
| 1636 | E_{1b}Eᵢ | AlkLEᵥᵢᵢ | | E_{b} |
| 1637 | E₁ₐEᵢᵢ | AlkLEᵥᵢᵢ | | E_{b} |
| 1638 | E_{1b}Eᵢᵢ | AlkLEᵥᵢᵢ | | E_{b} |
| 1639 | E₁ₐEᵢᵢE_{iib} | AlkLEᵥᵢᵢ | | E_{b} |
| 1640 | E_{1b}EᵢᵢE_{iib} | AlkLEᵥᵢᵢ | | E_{b} |
| 1641 | E₁ₐE₄ | AlkLEᵥᵢᵢ | | E_{b} |
| 1642 | E_{1b}E₄ | AlkLEᵥᵢᵢ | | E_{b} |
| 1643 | E₁ₐEᵢE₄ | AlkLEᵥᵢᵢ | | E_{b} |
| 1644 | E_{1b}EᵢE₄ | AlkLEᵥᵢᵢ | | E_{b} |
| 1645 | E₁ₐEᵢᵢE₄ | AlkLEᵥᵢᵢ | | E_{b} |
| 1646 | E_{1b}EᵢᵢE₄ | AlkLEᵥᵢᵢ | | E_{b} |
| 1647 | E₁ₐEᵢᵢE_{iib}E₄ | AlkLEᵥᵢᵢ | | E_{b} |
| 1648 | E_{1b}EᵢᵢE_{iib}E₄ | AlkLEᵥᵢᵢ | | E_{b} |
| 1649 | E₁ₐEᵢᵢᵢ | AlkLEᵥᵢᵢ | | E_{b} |
| 1650 | E_{1b}Eᵢᵢᵢ | AlkLEᵥᵢᵢ | | E_{b} |
| 1651 | E₁ₐEᵢᵢᵢE₄ | AlkLEᵥᵢᵢ | | E_{b} |
| 1652 | E_{1b}EᵢᵢᵢE₄ | AlkLEᵥᵢᵢ | | E_{b} |
| 1653 | E₁ₐEᵢᵥ | AlkLEᵥᵢᵢ | | E_{b} |
| 1654 | E_{1b}Eᵢᵥ | AlkLEᵥᵢᵢ | | E_{b} |
| 1655 | E₁ₐEᵢᵥE₄ | AlkLEᵥᵢᵢ | | E_{b} |
| 1656 | E_{1b}EᵢᵥE₄ | AlkLEᵥᵢᵢ | | E_{b} |
| 1657 | E₁ₐ | AlkLEᵥᵢᵢ | E₄* | E_{b} |
| 1658 | E_{1b} | AlkLEᵥᵢᵢ | E₄* | E_{b} |
| 1659 | E₁ₐEᵢ | AlkLEᵥᵢᵢ | E₄* | E_{b} |
| 1660 | E_{1b}Eᵢ | AlkLEᵥᵢᵢ | E₄* | E_{b} |
| 1661 | E₁ₐEᵢᵢ | AlkLEᵥᵢᵢ | E₄* | E_{b} |
| 1662 | E_{1b}Eᵢᵢ | AlkLEᵥᵢᵢ | E₄* | E_{b} |
| 1663 | E₁ₐEᵢᵢE_{iib} | AlkLEᵥᵢᵢ | E₄* | E_{b} |
| 1664 | E_{1b}EᵢᵢE_{iib} | AlkLEᵥᵢᵢ | E₄* | E_{b} |
| 1665 | E₁ₐE₄ | AlkLEᵥᵢᵢ | E₄* | E_{b} |
| 1666 | E_{1b}E₄ | AlkLEᵥᵢᵢ | E₄* | E_{b} |
| 1667 | E₁ₐEᵢE₄ | AlkLEᵥᵢᵢ | E₄* | E_{b} |
| 1668 | E_{1b}EᵢE₄ | AlkLEᵥᵢᵢ | E₄* | E_{b} |
| 1669 | E₁ₐEᵢᵢE₄ | AlkLEᵥᵢᵢ | E₄* | E_{b} |
| 1670 | E_{1b}EᵢᵢE₄ | AlkLEᵥᵢᵢ | E₄* | E_{b} |
| 1671 | E₁ₐEᵢᵢE_{iib}E₄ | AlkLEᵥᵢᵢ | E₄* | E_{b} |
| 1672 | E_{1b}EᵢᵢE_{iib}E₄ | AlkLEᵥᵢᵢ | E₄* | E_{b} |
| 1673 | E₁ₐEᵢᵢᵢ | AlkLEᵥᵢᵢ | E₄* | E_{b} |
| 1674 | E_{1b}Eᵢᵢᵢ | AlkLEᵥᵢᵢ | E₄* | E_{b} |
| 1675 | E₁ₐEᵢᵢᵢE₄ | AlkLEᵥᵢᵢ | E₄* | E_{b} |
| 1676 | E_{1b}EᵢᵢᵢE₄ | AlkLEᵥᵢᵢ | E₄* | E_{b} |
| 1677 | E₁ₐEᵢᵥ | AlkLEᵥᵢᵢ | E₄* | E_{b} |
| 1678 | E_{1b}Eᵢᵥ | AlkLEᵥᵢᵢ | E₄* | E_{b} |
| 1679 | E₁ₐEᵢᵥE₄ | AlkLEᵥᵢᵢ | E₄* | E_{b} |
| 1680 | E_{1b}EᵢᵥE₄ | AlkLEᵥᵢᵢ | E₄* | E_{b} |
| 1681 | E₁ₐ | AlkLEᵥᵢᵢ | E₅ | E_{b} |
| 1682 | E_{1b} | AlkLEᵥᵢᵢ | E₅ | E_{b} |
| 1683 | E₁ₐEᵢ | AlkLEᵥᵢᵢ | E₅ | E_{b} |
| 1684 | E_{1b}Eᵢ | AlkLEᵥᵢᵢ | E₅ | E_{b} |
| 1685 | E₁ₐEᵢᵢ | AlkLEᵥᵢᵢ | E₅ | E_{b} |
| 1686 | E_{1b}Eᵢᵢ | AlkLEᵥᵢᵢ | E₅ | E_{b} |
| 1687 | E₁ₐEᵢᵢE_{iib} | AlkLEᵥᵢᵢ | E₅ | E_{b} |
| 1688 | E_{1b}EᵢᵢE_{iib} | AlkLEᵥᵢᵢ | E₅ | E_{b} |
| 1689 | E₁ₐE₄ | AlkLEᵥᵢᵢ | E₅ | E_{b} |
| 1690 | E_{1b}E₄ | AlkLEᵥᵢᵢ | E₅ | E_{b} |
| 1691 | E₁ₐEᵢE₄ | AlkLEᵥᵢᵢ | E₅ | E_{b} |
| 1692 | E_{1b}EᵢE₄ | AlkLEᵥᵢᵢ | E₅ | E_{b} |
| 1693 | E₁ₐEᵢᵢE₄ | AlkLEᵥᵢᵢ | E₅ | E_{b} |
| 1694 | E_{1b}EᵢᵢE₄ | AlkLEᵥᵢᵢ | E₅ | E_{b} |
| 1695 | E₁ₐEᵢᵢE_{iib}E₄ | AlkLEᵥᵢᵢ | E₅ | E_{b} |
| 1696 | E_{1b}EᵢᵢE_{iib}E₄ | AlkLEᵥᵢᵢ | E₅ | E_{b} |
| 1697 | E₁ₐEᵢᵢᵢ | AlkLEᵥᵢᵢ | E₅ | E_{b} |
| 1698 | E_{1b}Eᵢᵢᵢ | AlkLEᵥᵢᵢ | E₅ | E_{b} |
| 1699 | E₁ₐEᵢᵢᵢE₄ | AlkLEᵥᵢᵢ | E₅ | E_{b} |
| 1700 | E_{1b}EᵢᵢᵢE₄ | AlkLEᵥᵢᵢ | E₅ | E_{b} |
| 1701 | E₁ₐEᵢᵥ | AlkLEᵥᵢᵢ | E₅ | E_{b} |
| 1702 | E_{1b}Eᵢᵥ | AlkLEᵥᵢᵢ | E₅ | E_{b} |
| 1703 | E₁ₐEᵢᵥE₄ | AlkLEᵥᵢᵢ | E₅ | E_{b} |
| 1704 | E_{1b}EᵢᵥE₄ | AlkLEᵥᵢᵢ | E₅ | E_{b} |
| 1705 | E₁ₐ | AlkLEᵥᵢᵢ | E₄*E₅ | E_{b} |
| 1706 | E_{1b} | AlkLEᵥᵢᵢ | E₄*E₅ | E_{b} |
| 1707 | E₁ₐEᵢ | AlkLEᵥᵢᵢ | E₄*E₅ | E_{b} |
| 1708 | E_{1b}Eᵢ | AlkLEᵥᵢᵢ | E₄*E₅ | E_{b} |
| 1709 | E₁ₐEᵢᵢ | AlkLEᵥᵢᵢ | E₄*E₅ | E_{b} |
| 1710 | E_{1b}Eᵢᵢ | AlkLEᵥᵢᵢ | E₄*E₅ | E_{b} |
| 1711 | E₁ₐEᵢᵢE_{iib} | AlkLEᵥᵢᵢ | E₄*E₅ | E_{b} |
| 1712 | E_{1b}EᵢᵢE_{iib} | AlkLEᵥᵢᵢ | E₄*E₅ | E_{b} |
| 1713 | E₁ₐE₄ | AlkLEᵥᵢᵢ | E₄*E₅ | E_{b} |
| 1714 | E_{1b}E₄ | AlkLEᵥᵢᵢ | E₄*E₅ | E_{b} |
| 1715 | E₁ₐEᵢE₄ | AlkLEᵥᵢᵢ | E₄*E₅ | E_{b} |
| 1716 | E_{1b}EᵢE₄ | AlkLEᵥᵢᵢ | E₄*E₅ | E_{b} |
| 1717 | E₁ₐEᵢᵢE₄ | AlkLEᵥᵢᵢ | E₄*E₅ | E_{b} |
| 1718 | E_{1b}EᵢᵢE₄ | AlkLEᵥᵢᵢ | E₄*E₅ | E_{b} |
| 1719 | E₁ₐEᵢᵢE_{iib}E₄ | AlkLEᵥᵢᵢ | E₄*E₅ | E_{b} |
| 1720 | E_{1b}EᵢᵢE_{iib}E₄ | AlkLEᵥᵢᵢ | E₄*E₅ | E_{b} |
| 1721 | E₁ₐEᵢᵢᵢ | AlkLEᵥᵢᵢ | E₄*E₅ | E_{b} |
| 1722 | E_{1b}Eᵢᵢᵢ | AlkLEᵥᵢᵢ | E₄*E₅ | E_{b} |
| 1723 | E₁ₐEᵢᵢᵢE₄ | AlkLEᵥᵢᵢ | E₄*E₅ | E_{b} |
| 1724 | E_{1b}EᵢᵢᵢE₄ | AlkLEᵥᵢᵢ | E₄*E₅ | E_{b} |
| 1725 | E₁ₐEᵢᵥ | AlkLEᵥᵢᵢ | E₄*E₅ | E_{b} |
| 1726 | E_{1b}Eᵢᵥ | AlkLEᵥᵢᵢ | E₄*E₅ | E_{b} |
| 1727 | E₁ₐEᵢᵥE₄ | AlkLEᵥᵢᵢ | E₄*E₅ | E_{b} |
| 1728 | E_{1b}EᵢᵥE₄ | AlkLEᵥᵢᵢ | E₄*E₅ | E_{b} |
| 1729 | E₁ₐ | Eᵥᵢᵢ | | E_{d} |
| 1730 | E_{1b} | Eᵥᵢᵢ | | E_{d} |
| 1731 | E₁ₐEᵢ | Eᵥᵢᵢ | | E_{d} |
| 1732 | E_{1b}Eᵢ | Eᵥᵢᵢ | | E_{d} |
| 1733 | E₁ₐEᵢᵢ | Eᵥᵢᵢ | | E_{d} |
| 1734 | E_{1b}Eᵢᵢ | Eᵥᵢᵢ | | E_{d} |
| 1735 | E₁ₐEᵢᵢE_{iib} | Eᵥᵢᵢ | | E_{d} |
| 1736 | E_{1b}EᵢᵢE_{iib} | Eᵥᵢᵢ | | E_{d} |
| 1737 | E₁ₐE₄ | Eᵥᵢᵢ | | E_{d} |
| 1738 | E_{1b}E₄ | Eᵥᵢᵢ | | E_{d} |
| 1739 | E₁ₐEᵢE₄ | Eᵥᵢᵢ | | E_{d} |
| 1740 | E_{1b}EᵢE₄ | Eᵥᵢᵢ | | E_{d} |
| 1741 | E₁ₐEᵢᵢE₄ | Eᵥᵢᵢ | | E_{d} |
| 1742 | E_{1b}EᵢᵢE₄ | Eᵥᵢᵢ | | E_{d} |
| 1743 | E₁ₐEᵢᵢE_{iib}E₄ | Eᵥᵢᵢ | | E_{d} |
| 1744 | E_{1b}EᵢᵢE_{iib}E₄ | Eᵥᵢᵢ | | E_{d} |
| 1745 | E₁ₐEᵢᵢᵢ | Eᵥᵢᵢ | | E_{d} |
| 1746 | E_{1b}Eᵢᵢᵢ | Eᵥᵢᵢ | | E_{d} |
| 1747 | E₁ₐEᵢᵢᵢE₄ | Eᵥᵢᵢ | | E_{d} |
| 1748 | E_{1b}EᵢᵢᵢE₄ | Eᵥᵢᵢ | | E_{d} |
| 1749 | E₁ₐEᵢᵥ | Eᵥᵢᵢ | | E_{d} |
| 1750 | E_{1b}Eᵢᵥ | Eᵥᵢᵢ | | E_{d} |
| 1751 | E₁ₐEᵢᵥE₄ | Eᵥᵢᵢ | | E_{d} |
| 1752 | E_{1b}EᵢᵥE₄ | Eᵥᵢᵢ | | E_{d} |
| 1753 | E₁ₐ | Eᵥᵢᵢ | E₄* | E_{d} |
| 1754 | E_{1b} | Eᵥᵢᵢ | E₄* | E_{d} |
| 1755 | E₁ₐEᵢ | Eᵥᵢᵢ | E₄* | E_{d} |
| 1756 | E_{1b}Eᵢ | Eᵥᵢᵢ | E₄* | E_{d} |
| 1757 | E₁ₐEᵢᵢ | Eᵥᵢᵢ | E₄* | E_{d} |
| 1758 | E_{1b}Eᵢᵢ | Eᵥᵢᵢ | E₄* | E_{d} |
| 1759 | E₁ₐEᵢᵢE_{iib} | Eᵥᵢᵢ | E₄* | E_{d} |
| 1760 | E_{1b}EᵢᵢE_{iib} | Eᵥᵢᵢ | E₄* | E_{d} |
| 1761 | E₁ₐE₄ | Eᵥᵢᵢ | E₄* | E_{d} |
| 1762 | E_{1b}E₄ | Eᵥᵢᵢ | E₄* | E_{d} |
| 1763 | E₁ₐEᵢE₄ | Eᵥᵢᵢ | E₄* | E_{d} |
| 1764 | E_{1b}EᵢE₄ | Eᵥᵢᵢ | E₄* | E_{d} |
| 1765 | E₁ₐEᵢᵢE₄ | Eᵥᵢᵢ | E₄* | E_{d} |
| 1766 | E_{1b}EᵢᵢE₄ | Eᵥᵢᵢ | E₄* | E_{d} |
| 1767 | E₁ₐEᵢᵢE_{iib}E₄ | Eᵥᵢᵢ | E₄* | E_{d} |
| 1768 | E_{1b}EᵢᵢE_{iib}E₄ | Eᵥᵢᵢ | E₄* | E_{d} |
| 1769 | E₁ₐEᵢᵢᵢ | Eᵥᵢᵢ | E₄* | E_{d} |
| 1770 | E_{1b}Eᵢᵢᵢ | Eᵥᵢᵢ | E₄* | E_{d} |
| 1771 | E₁ₐEᵢᵢᵢE₄ | Eᵥᵢᵢ | E₄* | E_{d} |
| 1772 | E_{1b}EᵢᵢᵢE₄ | Eᵥᵢᵢ | E₄* | E_{d} |
| 1773 | E₁ₐEᵢᵥ | Eᵥᵢᵢ | E₄* | E_{d} |
| 1774 | E_{1b}Eᵢᵥ | Eᵥᵢᵢ | E₄* | E_{d} |
| 1775 | E₁ₐEᵢᵥE₄ | Eᵥᵢᵢ | E₄* | E_{d} |
| 1776 | E_{1b}EᵢᵥE₄ | Eᵥᵢᵢ | E₄* | E_{d} |
| 1777 | E₁ₐ | Eᵥᵢᵢ | E₅ | E_{d} |
| 1778 | E_{1b} | Eᵥᵢᵢ | E₅ | E_{d} |
| 1779 | E₁ₐEᵢ | Eᵥᵢᵢ | E₅ | E_{d} |
| 1780 | E_{1b}Eᵢ | Eᵥᵢᵢ | E₅ | E_{d} |
| 1781 | E₁ₐEᵢᵢ | Eᵥᵢᵢ | E₅ | E_{d} |
| 1782 | E_{1b}Eᵢᵢ | Eᵥᵢᵢ | E₅ | E_{d} |
| 1783 | E₁ₐEᵢᵢE_{iib} | Eᵥᵢᵢ | E₅ | E_{d} |
| 1784 | E_{1b}EᵢᵢE_{iib} | Eᵥᵢᵢ | E₅ | E_{d} |
| 1785 | E₁ₐE₄ | Eᵥᵢᵢ | E₅ | E_{d} |
| 1786 | E_{1b}E₄ | Eᵥᵢᵢ | E₅ | E_{d} |
| 1787 | E₁ₐEᵢE₄ | Eᵥᵢᵢ | E₅ | E_{d} |
| 1788 | E_{1b}EᵢE₄ | Eᵥᵢᵢ | E₅ | E_{d} |
| 1789 | E₁ₐEᵢᵢE₄ | Eᵥᵢᵢ | E₅ | E_{d} |
| 1790 | E_{1b}EᵢᵢE₄ | Eᵥᵢᵢ | E₅ | E_{d} |
| 1791 | E₁ₐEᵢᵢE_{iib}E₄ | Eᵥᵢᵢ | E₅ | E_{d} |
| 1792 | E_{1b}EᵢᵢE_{iib}E₄ | Eᵥᵢᵢ | E₅ | E_{d} |
| 1793 | E₁ₐEᵢᵢᵢ | Eᵥᵢᵢ | E₅ | E_{d} |
| 1794 | E_{1b}Eᵢᵢᵢ | Eᵥᵢᵢ | E₅ | E_{d} |
| 1795 | E₁ₐEᵢᵢᵢE₄ | Eᵥᵢᵢ | E₅ | E_{d} |
| 1796 | E_{1b}EᵢᵢᵢE₄ | Eᵥᵢᵢ | E₅ | E_{d} |
| 1797 | E₁ₐEᵢᵥ | Eᵥᵢᵢ | E₅ | E_{d} |
| 1798 | E_{1b}Eᵢᵥ | Eᵥᵢᵢ | E₅ | E_{d} |
| 1799 | E₁ₐEᵢᵥE₄ | Eᵥᵢᵢ | E₅ | E_{d} |
| 1800 | E_{1b}EᵢᵥE₄ | Eᵥᵢᵢ | E₅ | E_{d} |
| 1801 | E₁ₐ | Eᵥᵢᵢ | E₄*E₅ | E_{d} |
| 1802 | E_{1b} | Eᵥᵢᵢ | E₄*E₅ | E_{d} |
| 1803 | E₁ₐEᵢ | Eᵥᵢᵢ | E₄*E₅ | E_{d} |
| 1804 | E_{1b}Eᵢ | Eᵥᵢᵢ | E₄*E₅ | E_{d} |
| 1805 | E₁ₐEᵢᵢ | Eᵥᵢᵢ | E₄*E₅ | E_{d} |
| 1806 | E_{1b}Eᵢᵢ | Eᵥᵢᵢ | E₄*E₅ | E_{d} |
| 1807 | E₁ₐEᵢᵢE_{iib} | Eᵥᵢᵢ | E₄*E₅ | E_{d} |
| 1808 | E_{1b}EᵢᵢE_{iib} | Eᵥᵢᵢ | E₄*E₅ | E_{d} |
| 1809 | E₁ₐE₄ | Eᵥᵢᵢ | E₄*E₅ | E_{d} |
| 1810 | E_{1b}E₄ | Eᵥᵢᵢ | E₄*E₅ | E_{d} |
| 1811 | E₁ₐEᵢE₄ | Eᵥᵢᵢ | E₄*E₅ | E_{d} |
| 1812 | E_{1b}EᵢE₄ | Eᵥᵢᵢ | E₄*E₅ | E_{d} |
| 1813 | E₁ₐEᵢᵢE₄ | Eᵥᵢᵢ | E₄*E₅ | E_{d} |
| 1814 | E_{1b}EᵢᵢE₄ | Eᵥᵢᵢ | E₄*E₅ | E_{d} |
| 1815 | E₁ₐEᵢᵢE_{iib}E₄ | Eᵥᵢᵢ | E₄*E₅ | E_{d} |
| 1816 | E_{1b}EᵢᵢE_{iib}E₄ | Eᵥᵢᵢ | E₄*E₅ | E_{d} |
| 1817 | E₁ₐEᵢᵢᵢ | Eᵥᵢᵢ | E₄*E₅ | E_{d} |
| 1818 | E_{1b}Eᵢᵢᵢ | Eᵥᵢᵢ | E₄*E₅ | E_{d} |
| 1819 | E₁ₐEᵢᵢᵢE₄ | Eᵥᵢᵢ | E₄*E₅ | E_{d} |
| 1820 | E_{1b}EᵢᵢᵢE₄ | Eᵥᵢᵢ | E₄*E₅ | E_{d} |
| 1821 | E₁ₐEᵢᵥ | Eᵥᵢᵢ | E₄*E₅ | E_{d} |
| 1822 | E_{1b}Eᵢᵥ | Eᵥᵢᵢ | E₄*E₅ | E_{d} |
| 1823 | E₁ₐEᵢᵥE₄ | Eᵥᵢᵢ | E₄*E₅ | E_{d} |
| 1824 | E_{1b}EᵢᵥE₄ | Eᵥᵢᵢ | E₄*E₅ | E_{d} |
| 1825 | E₁ₐ | AlkLEᵥᵢᵢ | | E_{d} |
| 1826 | E_{1b} | AlkLEᵥᵢᵢ | | E_{d} |
| 1827 | E₁ₐEᵢ | AlkLEᵥᵢᵢ | | E_{d} |
| 1828 | E_{1b}Eᵢ | AlkLEᵥᵢᵢ | | E_{d} |
| 1829 | E₁ₐEᵢᵢ | AlkLEᵥᵢᵢ | | E_{d} |
| 1830 | E_{1b}Eᵢᵢ | AlkLEᵥᵢᵢ | | E_{d} |
| 1831 | E₁ₐEᵢᵢE_{iib} | AlkLEᵥᵢᵢ | | E_{d} |
| 1832 | E_{1b}EᵢᵢE_{iib} | AlkLEᵥᵢᵢ | | E_{d} |
| 1833 | E₁ₐE₄ | AlkLEᵥᵢᵢ | | E_{d} |
| 1834 | E_{1b}E₄ | AlkLEᵥᵢᵢ | | E_{d} |
| 1835 | E₁ₐEᵢE₄ | AlkLEᵥᵢᵢ | | E_{d} |
| 1836 | E_{1b}EᵢE₄ | AlkLEᵥᵢᵢ | | E_{d} |
| 1837 | E₁ₐEᵢᵢE₄ | AlkLEᵥᵢᵢ | | E_{d} |
| 1838 | E_{1b}EᵢᵢE₄ | AlkLEᵥᵢᵢ | | E_{d} |
| 1839 | E₁ₐEᵢᵢE_{iib}E₄ | AlkLEᵥᵢᵢ | | E_{d} |
| 1840 | E_{1b}EᵢᵢE_{iib}E₄ | AlkLEᵥᵢᵢ | | E_{d} |
| 1841 | E₁ₐEᵢᵢᵢ | AlkLEᵥᵢᵢ | | E_{d} |
| 1842 | E_{1b}Eᵢᵢᵢ | AlkLEᵥᵢᵢ | | E_{d} |
| 1843 | E₁ₐEᵢᵢᵢE₄ | AlkLEᵥᵢᵢ | | E_{d} |
| 1844 | E_{1b}EᵢᵢᵢE₄ | AlkLEᵥᵢᵢ | | E_{d} |
| 1845 | E₁ₐEᵢᵥ | AlkLEᵥᵢᵢ | | E_{d} |
| 1846 | E_{1b}Eᵢᵥ | AlkLEᵥᵢᵢ | | E_{d} |
| 1847 | E₁ₐEᵢᵥE₄ | AlkLEᵥᵢᵢ | | E_{d} |
| 1848 | E_{1b}EᵢᵥE₄ | AlkLEᵥᵢᵢ | | E_{d} |
| 1849 | E₁ₐ | AlkLEᵥᵢᵢ | E₄* | E_{d} |
| 1850 | E_{1b} | AlkLEᵥᵢᵢ | E₄* | E_{d} |
| 1851 | E₁ₐEᵢ | AlkLEᵥᵢᵢ | E₄* | E_{d} |
| 1852 | E_{1b}Eᵢ | AlkLEᵥᵢᵢ | E₄* | E_{d} |
| 1853 | E₁ₐEᵢᵢ | AlkLEᵥᵢᵢ | E₄* | E_{d} |
| 1854 | E_{1b}Eᵢᵢ | AlkLEᵥᵢᵢ | E₄* | E_{d} |
| 1855 | E₁ₐEᵢᵢE_{iib} | AlkLEᵥᵢᵢ | E₄* | E_{d} |
| 1856 | E_{1b}EᵢᵢE_{iib} | AlkLEᵥᵢᵢ | E₄* | E_{d} |
| 1857 | E₁ₐE₄ | AlkLEᵥᵢᵢ | E₄* | E_{d} |
| 1858 | E_{1b}E₄ | AlkLEᵥᵢᵢ | E₄* | E_{d} |
| 1859 | E₁ₐEᵢE₄ | AlkLEᵥᵢᵢ | E₄* | E_{d} |
| 1860 | E_{1b}EᵢE₄ | AlkLEᵥᵢᵢ | E₄* | E_{d} |
| 1861 | E₁ₐEᵢᵢE₄ | AlkLEᵥᵢᵢ | E₄* | E_{d} |
| 1862 | E_{1b}EᵢᵢE₄ | AlkLEᵥᵢᵢ | E₄* | E_{d} |
| 1863 | E₁ₐEᵢᵢE_{iib}E₄ | AlkLEᵥᵢᵢ | E₄* | E_{d} |
| 1864 | E_{1b}EᵢᵢE_{iib}E₄ | AlkLEᵥᵢᵢ | E₄* | E_{d} |
| 1865 | E₁ₐEᵢᵢᵢ | AlkLEᵥᵢᵢ | E₄* | E_{d} |
| 1866 | E_{1b}Eᵢᵢᵢ | AlkLEᵥᵢᵢ | E₄* | E_{d} |
| 1867 | E₁ₐEᵢᵢᵢE₄ | AlkLEᵥᵢᵢ | E₄* | E_{d} |
| 1868 | E_{1b}EᵢᵢᵢE₄ | AlkLEᵥᵢᵢ | E₄* | E_{d} |
| 1869 | E₁ₐEᵢᵥ | AlkLEᵥᵢᵢ | E₄* | E_{d} |
| 1870 | E_{1b}Eᵢᵥ | AlkLEᵥᵢᵢ | E₄* | E_{d} |
| 1871 | E₁ₐEᵢᵥE₄ | AlkLEᵥᵢᵢ | E₄* | E_{d} |
| 1872 | E_{1b}EᵢᵥE₄ | AlkLEᵥᵢᵢ | E₄* | E_{d} |
| 1873 | E₁ₐ | AlkLEᵥᵢᵢ | E₅ | E_{d} |
| 1874 | E_{1b} | AlkLEᵥᵢᵢ | E₅ | E_{d} |
| 1875 | E₁ₐEᵢ | AlkLEᵥᵢᵢ | E₅ | E_{d} |
| 1876 | E_{1b}Eᵢ | AlkLEᵥᵢᵢ | E₅ | E_{d} |
| 1877 | E₁ₐEᵢᵢ | AlkLEᵥᵢᵢ | E₅ | E_{d} |
| 1878 | E_{1b}Eᵢᵢ | AlkLEᵥᵢᵢ | E₅ | E_{d} |
| 1879 | E₁ₐEᵢᵢE_{iib} | AlkLEᵥᵢᵢ | E₅ | E_{d} |
| 1880 | E_{1b}EᵢᵢE_{iib} | AlkLEᵥᵢᵢ | E₅ | E_{d} |
| 1881 | E₁ₐE₄ | AlkLEᵥᵢᵢ | E₅ | E_{d} |
| 1882 | E_{1b}E₄ | AlkLEᵥᵢᵢ | E₅ | E_{d} |
| 1883 | E₁ₐEᵢE₄ | AlkLEᵥᵢᵢ | E₅ | E_{d} |
| 1884 | E_{1b}EᵢE₄ | AlkLEᵥᵢᵢ | E₅ | E_{d} |
| 1885 | E₁ₐEᵢᵢE₄ | AlkLEᵥᵢᵢ | E₅ | E_{d} |
| 1886 | E_{1b}EᵢᵢE₄ | AlkLEᵥᵢᵢ | E₅ | E_{d} |
| 1887 | E₁ₐEᵢᵢE_{iib}E₄ | AlkLEᵥᵢᵢ | E₅ | E_{d} |
| 1888 | E_{1b}EᵢᵢE_{iib}E₄ | AlkLEᵥᵢᵢ | E₅ | E_{d} |
| 1889 | E₁ₐEᵢᵢᵢ | AlkLEᵥᵢᵢ | E₅ | E_{d} |
| 1890 | E_{1b}Eᵢᵢᵢ | AlkLEᵥᵢᵢ | E₅ | E_{d} |
| 1891 | E₁ₐEᵢᵢᵢE₄ | AlkLEᵥᵢᵢ | E₅ | E_{d} |
| 1892 | E_{1b}EᵢᵢᵢE₄ | AlkLEᵥᵢᵢ | E₅ | E_{d} |
| 1893 | E₁ₐEᵢᵥ | AlkLEᵥᵢᵢ | E₅ | E_{d} |
| 1894 | E_{1b}Eᵢᵥ | AlkLEᵥᵢᵢ | E₅ | E_{d} |
| 1895 | E₁ₐEᵢᵥE₄ | AlkLEᵥᵢᵢ | E₅ | E_{d} |
| 1896 | E_{1b}EᵢᵥE₄ | AlkLEᵥᵢᵢ | E₅ | E_{d} |
| 1897 | E₁ₐ | AlkLEᵥᵢᵢ | E₄*E₅ | E_{d} |
| 1898 | E_{1b} | AlkLEᵥᵢᵢ | E₄*E₅ | E_{d} |
| 1899 | E₁ₐEᵢ | AlkLEᵥᵢᵢ | E₄*E₅ | E_{d} |
| 1900 | E_{1b}Eᵢ | AlkLEᵥᵢᵢ | E₄*E₅ | E_{d} |
| 1901 | E₁ₐEᵢᵢ | AlkLEᵥᵢᵢ | E₄*E₅ | E_{d} |
| 1902 | E_{1b}Eᵢᵢ | AlkLEᵥᵢᵢ | E₄*E₅ | E_{d} |
| 1903 | E₁ₐEᵢᵢE_{iib} | AlkLEᵥᵢᵢ | E₄*E₅ | E_{d} |
| 1904 | E_{1b}EᵢᵢE_{iib} | AlkLEᵥᵢᵢ | E₄*E₅ | E_{d} |
| 1905 | E₁ₐE₄ | AlkLEᵥᵢᵢ | E₄*E₅ | E_{d} |
| 1906 | E_{1b}E₄ | AlkLEᵥᵢᵢ | E₄*E₅ | E_{d} |
| 1907 | E₁ₐEᵢE₄ | AlkLEᵥᵢᵢ | E₄*E₅ | E_{d} |
| 1908 | E_{1b}EᵢE₄ | AlkLEᵥᵢᵢ | E₄*E₅ | E_{d} |
| 1909 | E₁ₐEᵢᵢE₄ | AlkLEᵥᵢᵢ | E₄*E₅ | E_{d} |
| 1910 | E_{1b}EᵢᵢE₄ | AlkLEᵥᵢᵢ | E₄*E₅ | E_{d} |
| 1911 | E₁ₐEᵢᵢE_{iib}E₄ | AlkLEᵥᵢᵢ | E₄*E₅ | E_{d} |
| 1912 | E_{1b}EᵢᵢE_{iib}E₄ | AlkLEᵥᵢᵢ | E₄*E₅ | E_{d} |
| 1913 | E₁ₐEᵢᵢᵢ | AlkLEᵥᵢᵢ | E₄*E₅ | E_{d} |
| 1914 | E_{1b}Eᵢᵢᵢ | AlkLEᵥᵢᵢ | E₄*E₅ | E_{d} |
| 1915 | E₁ₐEᵢᵢᵢE₄ | AlkLEᵥᵢᵢ | E₄*E₅ | E_{d} |
| 1916 | E_{1b}EᵢᵢᵢE₄ | AlkLEᵥᵢᵢ | E₄*E₅ | E_{d} |
| 1917 | E₁ₐEᵢᵥ | AlkLEᵥᵢᵢ | E₄*E₅ | E_{d} |
| 1918 | E_{1b}Eᵢᵥ | AlkLEᵥᵢᵢ | E₄*E₅ | E_{d} |
| 1919 | E₁ₐEᵢᵥE₄ | AlkLEᵥᵢᵢ | E₄*E₅ | E_{d} |
| 1920 | E_{1b}EᵢᵥE₄ | AlkLEᵥᵢᵢ | E₄*E₅ | E_{d} |
| 1921 | E₁ₐ | Eᵥᵢᵢ | | Eₑ |
| 1922 | E_{1b} | Eᵥᵢᵢ | | Eₑ |
| 1923 | E₁ₐEᵢ | Eᵥᵢᵢ | | Eₑ |
| 1924 | E_{1b}Eᵢ | Eᵥᵢᵢ | | Eₑ |
| 1925 | E₁ₐEᵢᵢ | Eᵥᵢᵢ | | Eₑ |
| 1926 | E_{1b}Eᵢᵢ | Eᵥᵢᵢ | | Eₑ |
| 1927 | E₁ₐEᵢᵢE_{iib} | Eᵥᵢᵢ | | Eₑ |
| 1928 | E_{1b}EᵢᵢE_{iib} | Eᵥᵢᵢ | | Eₑ |
| 1929 | E₁ₐE₄ | Eᵥᵢᵢ | | Eₑ |
| 1930 | E_{1b}E₄ | Eᵥᵢᵢ | | Eₑ |
| 1931 | E₁ₐEᵢE₄ | Eᵥᵢᵢ | | Eₑ |
| 1932 | E_{1b}EᵢE₄ | Eᵥᵢᵢ | | Eₑ |
| 1933 | E₁ₐEᵢᵢE₄ | Eᵥᵢᵢ | | Eₑ |
| 1934 | E_{1b}EᵢᵢE₄ | Eᵥᵢᵢ | | Eₑ |
| 1935 | E₁ₐEᵢᵢE_{iib}E₄ | Eᵥᵢᵢ | | Eₑ |
| 1936 | E_{1b}EᵢᵢE_{iib}E₄ | Eᵥᵢᵢ | | Eₑ |
| 1937 | E₁ₐEᵢᵢᵢ | Eᵥᵢᵢ | | Eₑ |
| 1938 | E_{1b}Eᵢᵢᵢ | Eᵥᵢᵢ | | Eₑ |
| 1939 | E₁ₐEᵢᵢᵢE₄ | Eᵥᵢᵢ | | Eₑ |
| 1940 | E_{1b}EᵢᵢᵢE₄ | Eᵥᵢᵢ | | Eₑ |
| 1941 | E₁ₐEᵢᵥ | Eᵥᵢᵢ | | Eₑ |
| 1942 | E_{1b}Eᵢᵥ | Eᵥᵢᵢ | | Eₑ |
| 1943 | E₁ₐEᵢᵥE₄ | Eᵥᵢᵢ | | Eₑ |
| 1944 | E_{1b}EᵢᵥE₄ | Eᵥᵢᵢ | | Eₑ |
| 1945 | E₁ₐ | Eᵥᵢᵢ | E₄* | Eₑ |
| 1946 | E_{1b} | Eᵥᵢᵢ | E₄* | Eₑ |
| 1947 | E₁ₐEᵢ | Eᵥᵢᵢ | E₄* | Eₑ |
| 1948 | E_{1b}Eᵢ | Eᵥᵢᵢ | E₄* | Eₑ |
| 1949 | E₁ₐEᵢᵢ | Eᵥᵢᵢ | E₄* | Eₑ |
| 1950 | E_{1b}Eᵢᵢ | Eᵥᵢᵢ | E₄* | Eₑ |
| 1951 | E₁ₐEᵢᵢE_{iib} | Eᵥᵢᵢ | E₄* | Eₑ |
| 1952 | E_{1b}EᵢᵢE_{iib} | Eᵥᵢᵢ | E₄* | Eₑ |
| 1953 | E₁ₐE₄ | Eᵥᵢᵢ | E₄* | Eₑ |
| 1954 | E_{1b}E₄ | Eᵥᵢᵢ | E₄* | Eₑ |
| 1955 | E₁ₐEᵢE₄ | Eᵥᵢᵢ | E₄* | Eₑ |
| 1956 | E_{1b}EᵢE₄ | Eᵥᵢᵢ | E₄* | Eₑ |
| 1957 | E₁ₐEᵢᵢE₄ | Eᵥᵢᵢ | E₄* | Eₑ |
| 1958 | E_{1b}EᵢᵢE₄ | Eᵥᵢᵢ | E₄* | Eₑ |
| 1959 | E₁ₐEᵢᵢE_{iib}E₄ | Eᵥᵢᵢ | E₄* | Eₑ |
| 1960 | E_{1b}EᵢᵢE_{iib}E₄ | Eᵥᵢᵢ | E₄* | Eₑ |
| 1961 | E₁ₐEᵢᵢᵢ | Eᵥᵢᵢ | E₄* | Eₑ |
| 1962 | E_{1b}Eᵢᵢᵢ | Eᵥᵢᵢ | E₄* | Eₑ |
| 1963 | E₁ₐEᵢᵢᵢE₄ | Eᵥᵢᵢ | E₄* | Eₑ |
| 1964 | E_{1b}EᵢᵢᵢE₄ | Eᵥᵢᵢ | E₄* | Eₑ |
| 1965 | E₁ₐEᵢᵥ | Eᵥᵢᵢ | E₄* | Eₑ |
| 1966 | E_{1b}Eᵢᵥ | Eᵥᵢᵢ | E₄* | Eₑ |
| 1967 | E₁ₐEᵢᵥE₄ | Eᵥᵢᵢ | E₄* | Eₑ |
| 1968 | E_{1b}EᵢᵥE₄ | Eᵥᵢᵢ | E₄* | Eₑ |
| 1969 | E₁ₐ | Eᵥᵢᵢ | E₅ | Eₑ |
| 1970 | E_{1b} | Eᵥᵢᵢ | E₅ | Eₑ |
| 1971 | E₁ₐEᵢ | Eᵥᵢᵢ | E₅ | Eₑ |
| 1972 | E_{1b}Eᵢ | Eᵥᵢᵢ | E₅ | Eₑ |
| 1973 | E₁ₐEᵢᵢ | Eᵥᵢᵢ | E₅ | Eₑ |
| 1974 | E_{1b}Eᵢᵢ | Eᵥᵢᵢ | E₅ | Eₑ |
| 1975 | E₁ₐEᵢᵢE_{iib} | Eᵥᵢᵢ | E₅ | Eₑ |
| 1976 | E_{1b}EᵢᵢE_{iib} | Eᵥᵢᵢ | E₅ | Eₑ |
| 1977 | E₁ₐE₄ | Eᵥᵢᵢ | E₅ | Eₑ |
| 1978 | E_{1b}E₄ | Eᵥᵢᵢ | E₅ | Eₑ |
| 1979 | E₁ₐEᵢE₄ | Eᵥᵢᵢ | E₅ | Eₑ |
| 1980 | E_{1b}EᵢE₄ | Eᵥᵢᵢ | E₅ | Eₑ |
| 1981 | E₁ₐEᵢᵢE₄ | Eᵥᵢᵢ | E₅ | Eₑ |
| 1982 | E_{1b}EᵢᵢE₄ | Eᵥᵢᵢ | E₅ | Eₑ |
| 1983 | E₁ₐEᵢᵢE_{iib}E₄ | Eᵥᵢᵢ | E₅ | Eₑ |
| 1984 | E_{1b}EᵢᵢE_{iib}E₄ | Eᵥᵢᵢ | E₅ | Eₑ |
| 1985 | E₁ₐEᵢᵢᵢ | Eᵥᵢᵢ | E₅ | Eₑ |
| 1986 | E_{1b}Eᵢᵢᵢ | Eᵥᵢᵢ | E₅ | Eₑ |
| 1987 | E₁ₐEᵢᵢᵢE₄ | Eᵥᵢᵢ | E₅ | Eₑ |
| 1988 | E_{1b}EᵢᵢᵢE₄ | Eᵥᵢᵢ | E₅ | Eₑ |
| 1989 | E₁ₐEᵢᵥ | Eᵥᵢᵢ | E₅ | Eₑ |
| 1990 | E_{1b}Eᵢᵥ | Eᵥᵢᵢ | E₅ | Eₑ |
| 1991 | E₁ₐEᵢᵥE₄ | Eᵥᵢᵢ | E₅ | Eₑ |
| 1992 | E_{1b}EᵢᵥE₄ | Eᵥᵢᵢ | E₅ | Eₑ |
| 1993 | E₁ₐ | Eᵥᵢᵢ | E₄*E₅ | Eₑ |
| 1994 | E_{1b} | Eᵥᵢᵢ | E₄*E₅ | Eₑ |
| 1995 | E₁ₐEᵢ | Eᵥᵢᵢ | E₄*E₅ | Eₑ |
| 1996 | E_{1b}Eᵢ | Eᵥᵢᵢ | E₄*E₅ | Eₑ |
| 1997 | E₁ₐEᵢᵢ | Eᵥᵢᵢ | E₄*E₅ | Eₑ |
| 1998 | E_{1b}Eᵢᵢ | Eᵥᵢᵢ | E₄*E₅ | Eₑ |
| 1999 | E₁ₐEᵢᵢE_{iib} | Eᵥᵢᵢ | E₄*E₅ | Eₑ |
| 2000 | E_{1b}EᵢᵢE_{iib} | Eᵥᵢᵢ | E₄*E₅ | Eₑ |
| 2001 | E₁ₐE₄ | Eᵥᵢᵢ | E₄*E₅ | Eₑ |
| 2002 | E_{1b}E₄ | Eᵥᵢᵢ | E₄*E₅ | Eₑ |
| 2003 | E₁ₐEᵢE₄ | Eᵥᵢᵢ | E₄*E₅ | Eₑ |
| 2004 | E_{1b}EᵢE₄ | Eᵥᵢᵢ | E₄*E₅ | Eₑ |
| 2005 | E₁ₐEᵢᵢE₄ | Eᵥᵢᵢ | E₄*E₅ | Eₑ |
| 2006 | E_{1b}EᵢᵢE₄ | Eᵥᵢᵢ | E₄*E₅ | Eₑ |
| 2007 | E₁ₐEᵢᵢE_{iib}E₄ | Eᵥᵢᵢ | E₄*E₅ | Eₑ |
| 2008 | E_{1b}EᵢᵢE_{iib}E₄ | Eᵥᵢᵢ | E₄*E₅ | Eₑ |
| 2009 | E₁ₐEᵢᵢᵢ | Eᵥᵢᵢ | E₄*E₅ | Eₑ |
| 2010 | E_{1b}Eᵢᵢᵢ | Eᵥᵢᵢ | E₄*E₅ | Eₑ |
| 2011 | E₁ₐEᵢᵢᵢE₄ | Eᵥᵢᵢ | E₄*E₅ | Eₑ |
| 2012 | E_{1b}EᵢᵢᵢE₄ | Eᵥᵢᵢ | E₄*E₅ | Eₑ |
| 2013 | E₁ₐEᵢᵥ | Eᵥᵢᵢ | E₄*E₅ | Eₑ |
| 2014 | E_{1b}Eᵢᵥ | Eᵥᵢᵢ | E₄*E₅ | Eₑ |
| 2015 | E₁ₐEᵢᵥE₄ | Eᵥᵢᵢ | E₄*E₅ | Eₑ |
| 2016 | E_{1b}EᵢᵥE₄ | Eᵥᵢᵢ | E₄*E₅ | Eₑ |
| 2017 | E₁ₐ | AlkLEᵥᵢᵢ | | Eₑ |
| 2018 | E_{1b} | AlkLEᵥᵢᵢ | | Eₑ |
| 2019 | E₁ₐEᵢ | AlkLEᵥᵢᵢ | | Eₑ |
| 2020 | E_{1b}Eᵢ | AlkLEᵥᵢᵢ | | Eₑ |
| 2021 | E₁ₐEᵢᵢ | AlkLEᵥᵢᵢ | | Eₑ |
| 2022 | E_{1b}Eᵢᵢ | AlkLEᵥᵢᵢ | | Eₑ |
| 2023 | E₁ₐEᵢᵢE_{iib} | AlkLEᵥᵢᵢ | | Eₑ |
| 2024 | E_{1b}EᵢᵢE_{iib} | AlkLEᵥᵢᵢ | | Eₑ |
| 2025 | E₁ₐE₄ | AlkLEᵥᵢᵢ | | Eₑ |
| 2026 | E_{1b}E₄ | AlkLEᵥᵢᵢ | | Eₑ |
| 2027 | E₁ₐEᵢE₄ | AlkLEᵥᵢᵢ | | Eₑ |
| 2028 | E_{1b}EᵢE₄ | AlkLEᵥᵢᵢ | | Eₑ |
| 2029 | E₁ₐEᵢᵢE₄ | AlkLEᵥᵢᵢ | | Eₑ |
| 2030 | E_{1b}EᵢᵢE₄ | AlkLEᵥᵢᵢ | | Eₑ |
| 2031 | E₁ₐEᵢᵢE_{iib}E₄ | AlkLEᵥᵢᵢ | | Eₑ |
| 2032 | E_{1b}EᵢᵢE_{iib}E₄ | AlkLEᵥᵢᵢ | | Eₑ |
| 2033 | E₁ₐEᵢᵢᵢ | AlkLEᵥᵢᵢ | | Eₑ |
| 2034 | E_{1b}Eᵢᵢᵢ | AlkLEᵥᵢᵢ | | Eₑ |
| 2035 | E₁ₐEᵢᵢᵢE₄ | AlkLEᵥᵢᵢ | | Eₑ |
| 2036 | E_{1b}EᵢᵢᵢE₄ | AlkLEᵥᵢᵢ | | Eₑ |
| 2037 | E₁ₐEᵢᵥ | AlkLEᵥᵢᵢ | | Eₑ |
| 2038 | E_{1b}Eᵢᵥ | AlkLEᵥᵢᵢ | | Eₑ |
| 2039 | E₁ₐEᵢᵥE₄ | AlkLEᵥᵢᵢ | | Eₑ |
| 2040 | E_{1b}EᵢᵥE₄ | AlkLEᵥᵢᵢ | | Eₑ |
| 2041 | E₁ₐ | AlkLEᵥᵢᵢ | E₄* | Eₑ |
| 2042 | E_{1b} | AlkLEᵥᵢᵢ | E₄* | Eₑ |
| 2043 | E₁ₐEᵢ | AlkLEᵥᵢᵢ | E₄* | Eₑ |
| 2044 | E_{1b}Eᵢ | AlkLEᵥᵢᵢ | E₄* | Eₑ |
| 2045 | E₁ₐEᵢᵢ | AlkLEᵥᵢᵢ | E₄* | Eₑ |
| 2046 | E_{1b}Eᵢᵢ | AlkLEᵥᵢᵢ | E₄* | Eₑ |
| 2047 | E₁ₐEᵢᵢE_{iib} | AlkLEᵥᵢᵢ | E₄* | Eₑ |
| 2048 | E_{1b}EᵢᵢE_{iib} | AlkLEᵥᵢᵢ | E₄* | Eₑ |
| 2049 | E₁ₐE₄ | AlkLEᵥᵢᵢ | E₄* | Eₑ |
| 2050 | E_{1b}E₄ | AlkLEᵥᵢᵢ | E₄* | Eₑ |
| 2051 | E₁ₐEᵢE₄ | AlkLEᵥᵢᵢ | E₄* | Eₑ |
| 2052 | E_{1b}EᵢE₄ | AlkLEᵥᵢᵢ | E₄* | Eₑ |
| 2053 | E₁ₐEᵢᵢE₄ | AlkLEᵥᵢᵢ | E₄* | Eₑ |
| 2054 | E_{1b}EᵢᵢE₄ | AlkLEᵥᵢᵢ | E₄* | Eₑ |
| 2055 | E₁ₐEᵢᵢE_{iib}E₄ | AlkLEᵥᵢᵢ | E₄* | Eₑ |
| 2056 | E_{1b}EᵢᵢE_{iib}E₄ | AlkLEᵥᵢᵢ | E₄* | Eₑ |
| 2057 | E₁ₐEᵢᵢᵢ | AlkLEᵥᵢᵢ | E₄* | Eₑ |
| 2058 | E_{1b}Eᵢᵢᵢ | AlkLEᵥᵢᵢ | E₄* | Eₑ |
| 2059 | E₁ₐEᵢᵢᵢE₄ | AlkLEᵥᵢᵢ | E₄* | Eₑ |
| 2060 | E_{1b}EᵢᵢᵢE₄ | AlkLEᵥᵢᵢ | E₄* | Eₑ |
| 2061 | E₁ₐEᵢᵥ | AlkLEᵥᵢᵢ | E₄* | Eₑ |
| 2062 | E_{1b}Eᵢᵥ | AlkLEᵥᵢᵢ | E₄* | Eₑ |
| 2063 | E₁ₐEᵢᵥE₄ | AlkLEᵥᵢᵢ | E₄* | Eₑ |
| 2064 | E_{1b}EᵢᵥE₄ | AlkLEᵥᵢᵢ | E₄* | Eₑ |
| 2065 | E₁ₐ | AlkLEᵥᵢᵢ | E₅ | Eₑ |
| 2066 | E_{1b} | AlkLEᵥᵢᵢ | E₅ | Eₑ |
| 2067 | E₁ₐEᵢ | AlkLEᵥᵢᵢ | E₅ | Eₑ |
| 2068 | E_{1b}Eᵢ | AlkLEᵥᵢᵢ | E₅ | Eₑ |
| 2069 | E₁ₐEᵢᵢ | AlkLEᵥᵢᵢ | E₅ | Eₑ |
| 2070 | E_{1b}Eᵢᵢ | AlkLEᵥᵢᵢ | E₅ | Eₑ |
| 2071 | E₁ₐEᵢᵢE_{iib} | AlkLEᵥᵢᵢ | E₅ | Eₑ |
| 2072 | E_{1b}EᵢᵢE_{iib} | AlkLEᵥᵢᵢ | E₅ | Eₑ |
| 2073 | E₁ₐE₄ | AlkLEᵥᵢᵢ | E₅ | Eₑ |
| 2074 | E_{1b}E₄ | AlkLEᵥᵢᵢ | E₅ | Eₑ |
| 2075 | E₁ₐEᵢE₄ | AlkLEᵥᵢᵢ | E₅ | Eₑ |
| 2076 | E_{1b}EᵢE₄ | AlkLEᵥᵢᵢ | E₅ | Eₑ |
| 2077 | E₁ₐEᵢᵢE₄ | AlkLEᵥᵢᵢ | E₅ | Eₑ |
| 2078 | E_{1b}EᵢᵢE₄ | AlkLEᵥᵢᵢ | E₅ | Eₑ |
| 2079 | E₁ₐEᵢᵢE_{iib}E₄ | AlkLEᵥᵢᵢ | E₅ | Eₑ |
| 2080 | E_{1b}EᵢᵢE_{iib}E₄ | AlkLEᵥᵢᵢ | E₅ | Eₑ |
| 2081 | E₁ₐEᵢᵢᵢ | AlkLEᵥᵢᵢ | E₅ | Eₑ |
| 2082 | E_{1b}Eᵢᵢᵢ | AlkLEᵥᵢᵢ | E₅ | Eₑ |
| 2083 | E₁ₐEᵢᵢᵢE₄ | AlkLEᵥᵢᵢ | E₅ | Eₑ |
| 2084 | E_{1b}EᵢᵢᵢE₄ | AlkLEᵥᵢᵢ | E₅ | Eₑ |
| 2085 | E₁ₐEᵢᵥ | AlkLEᵥᵢᵢ | E₅ | Eₑ |
| 2086 | E_{1b}Eᵢᵥ | AlkLEᵥᵢᵢ | E₅ | Eₑ |
| 2087 | E₁ₐEᵢᵥE₄ | AlkLEᵥᵢᵢ | E₅ | Eₑ |
| 2088 | E_{1b}EᵢᵥE₄ | AlkLEᵥᵢᵢ | E₅ | Eₑ |
| 2089 | E₁ₐ | AlkLEᵥᵢᵢ | E₄*E₅ | Eₑ |
| 2090 | E_{1b} | AlkLEᵥᵢᵢ | E₄*E₅ | Eₑ |
| 2091 | E₁ₐEᵢ | AlkLEᵥᵢᵢ | E₄*E₅ | Eₑ |
| 2092 | E_{1b}Eᵢ | AlkLEᵥᵢᵢ | E₄*E₅ | Eₑ |
| 2093 | E₁ₐEᵢᵢ | AlkLEᵥᵢᵢ | E₄*E₅ | Eₑ |
| 2094 | E_{1b}Eᵢᵢ | AlkLEᵥᵢᵢ | E₄*E₅ | Eₑ |
| 2095 | E₁ₐEᵢᵢE_{iib} | AlkLEᵥᵢᵢ | E₄*E₅ | Eₑ |
| 2096 | E_{1b}EᵢᵢE_{iib} | AlkLEᵥᵢᵢ | E₄*E₅ | Eₑ |
| 2097 | E₁ₐE₄ | AlkLEᵥᵢᵢ | E₄*E₅ | Eₑ |
| 2098 | E_{1b}E₄ | AlkLEᵥᵢᵢ | E₄*E₅ | Eₑ |
| 2099 | E₁ₐEᵢE₄ | AlkLEᵥᵢᵢ | E₄*E₅ | Eₑ |
| 2100 | E_{1b}EᵢE₄ | AlkLEᵥᵢᵢ | E₄*E₅ | Eₑ |
| 2101 | E₁ₐEᵢᵢE₄ | AlkLEᵥᵢᵢ | E₄*E₅ | Eₑ |
| 2102 | E_{1b}EᵢᵢE₄ | AlkLEᵥᵢᵢ | E₄*E₅ | Eₑ |
| 2103 | E₁ₐEᵢᵢE_{iib}E₄ | AlkLEᵥᵢᵢ | E₄*E₅ | Eₑ |
| 2104 | E_{1b}EᵢᵢE_{iib}E₄ | AlkLEᵥᵢᵢ | E₄*E₅ | Eₑ |
| 2105 | E₁ₐEᵢᵢᵢ | AlkLEᵥᵢᵢ | E₄*E₅ | Eₑ |
| 2106 | E_{1b}Eᵢᵢᵢ | AlkLEᵥᵢᵢ | E₄*E₅ | Eₑ |
| 2107 | E₁ₐEᵢᵢᵢE₄ | AlkLEᵥᵢᵢ | E₄*E₅ | Eₑ |
| 2108 | E_{1b}EᵢᵢᵢE₄ | AlkLEᵥᵢᵢ | E₄*E₅ | Eₑ |
| 2109 | E₁ₐEᵢᵥ | AlkLEᵥᵢᵢ | E₄*E₅ | Eₑ |
| 2110 | E_{1b}Eᵢᵥ | AlkLEᵥᵢᵢ | E₄*E₅ | Eₑ |
| 2111 | E₁ₐEᵢᵥE₄ | AlkLEᵥᵢᵢ | E₄*E₅ | Eₑ |
| 2112 | E_{1b}EᵢᵥE₄ | AlkLEᵥᵢᵢ | E₄*E₅ | Eₑ |
| 2113 | E₁ₐ | Eᵥᵢᵢ | | E_{f} |
| 2114 | E_{1b} | Eᵥᵢᵢ | | E_{f} |
| 2115 | E₁ₐEᵢ | Eᵥᵢᵢ | | E_{f} |
| 2116 | E_{1b}Eᵢ | Eᵥᵢᵢ | | E_{f} |
| 2117 | E₁ₐEᵢᵢ | Eᵥᵢᵢ | | E_{f} |
| 2118 | E_{1b}Eᵢᵢ | Eᵥᵢᵢ | | E_{f} |
| 2119 | E₁ₐEᵢᵢE_{iib} | Eᵥᵢᵢ | | E_{f} |
| 2120 | E_{1b}EᵢᵢE_{iib} | Eᵥᵢᵢ | | E_{f} |
| 2121 | E₁ₐE₄ | Eᵥᵢᵢ | | E_{f} |
| 2122 | E_{1b}E₄ | Eᵥᵢᵢ | | E_{f} |
| 2123 | E₁ₐEᵢE₄ | Eᵥᵢᵢ | | E_{f} |
| 2124 | E_{1b}EᵢE₄ | Eᵥᵢᵢ | | E_{f} |
| 2125 | E₁ₐEᵢᵢE₄ | Eᵥᵢᵢ | | E_{f} |
| 2126 | E_{1b}EᵢᵢE₄ | Eᵥᵢᵢ | | E_{f} |
| 2127 | E₁ₐEᵢᵢE_{iib}E₄ | Eᵥᵢᵢ | | E_{f} |
| 2128 | E_{1b}EᵢᵢE_{iib}E₄ | Eᵥᵢᵢ | | E_{f} |
| 2129 | E₁ₐEᵢᵢᵢ | Eᵥᵢᵢ | | E_{f} |
| 2130 | E_{1b}Eᵢᵢᵢ | Eᵥᵢᵢ | | E_{f} |
| 2131 | E₁ₐEᵢᵢᵢE₄ | Eᵥᵢᵢ | | E_{f} |
| 2132 | E_{1b}EᵢᵢᵢE₄ | Eᵥᵢᵢ | | E_{f} |
| 2133 | E₁ₐEᵢᵥ | Eᵥᵢᵢ | | E_{f} |
| 2134 | E_{1b}Eᵢᵥ | Eᵥᵢᵢ | | E_{f} |
| 2135 | E₁ₐEᵢᵥE₄ | Eᵥᵢᵢ | | E_{f} |
| 2136 | E_{1b}EᵢᵥE₄ | Eᵥᵢᵢ | | E_{f} |
| 2137 | E₁ₐ | Eᵥᵢᵢ | E₄* | E_{f} |
| 2138 | E_{1b} | Eᵥᵢᵢ | E₄* | E_{f} |
| 2139 | E₁ₐEᵢ | Eᵥᵢᵢ | E₄* | E_{f} |
| 2140 | E_{1b}Eᵢ | Eᵥᵢᵢ | E₄* | E_{f} |
| 2141 | E₁ₐEᵢᵢ | Eᵥᵢᵢ | E₄* | E_{f} |
| 2142 | E_{1b}Eᵢᵢ | Eᵥᵢᵢ | E₄* | E_{f} |
| 2143 | E₁ₐEᵢᵢE_{iib} | Eᵥᵢᵢ | E₄* | E_{f} |
| 2144 | E_{1b}EᵢᵢE_{iib} | Eᵥᵢᵢ | E₄* | E_{f} |
| 2145 | E₁ₐE₄ | Eᵥᵢᵢ | E₄* | E_{f} |
| 2146 | E_{1b}E₄ | Eᵥᵢᵢ | E₄* | E_{f} |
| 2147 | E₁ₐEᵢE₄ | Eᵥᵢᵢ | E₄* | E_{f} |
| 2148 | E_{1b}EᵢE₄ | Eᵥᵢᵢ | E₄* | E_{f} |
| 2149 | E₁ₐEᵢᵢE₄ | Eᵥᵢᵢ | E₄* | E_{f} |
| 2150 | E_{1b}EᵢᵢE₄ | Eᵥᵢᵢ | E₄* | E_{f} |
| 2151 | E₁ₐEᵢᵢE_{iib}E₄ | Eᵥᵢᵢ | E₄* | E_{f} |
| 2152 | E_{1b}EᵢᵢE_{iib}E₄ | Eᵥᵢᵢ | E₄* | E_{f} |
| 2153 | E₁ₐEᵢᵢᵢ | Eᵥᵢᵢ | E₄* | E_{f} |
| 2154 | E_{1b}Eᵢᵢᵢ | Eᵥᵢᵢ | E₄* | E_{f} |
| 2155 | E₁ₐEᵢᵢᵢE₄ | Eᵥᵢᵢ | E₄* | E_{f} |
| 2156 | E_{1b}EᵢᵢᵢE₄ | Eᵥᵢᵢ | E₄* | E_{f} |
| 2157 | E₁ₐEᵢᵥ | Eᵥᵢᵢ | E₄* | E_{f} |
| 2158 | E_{1b}Eᵢᵥ | Eᵥᵢᵢ | E₄* | E_{f} |
| 2159 | E₁ₐEᵢᵥE₄ | Eᵥᵢᵢ | E₄* | E_{f} |
| 2160 | E_{1b}EᵢᵥE₄ | Eᵥᵢᵢ | E₄* | E_{f} |
| 2161 | E₁ₐ | Eᵥᵢᵢ | E₅ | E_{f} |
| 2162 | E_{1b} | Eᵥᵢᵢ | E₅ | E_{f} |
| 2163 | E₁ₐEᵢ | Eᵥᵢᵢ | E₅ | E_{f} |
| 2164 | E_{1b}Eᵢ | Eᵥᵢᵢ | E₅ | E_{f} |
| 2165 | E₁ₐEᵢᵢ | Eᵥᵢᵢ | E₅ | E_{f} |
| 2166 | E_{1b}Eᵢᵢ | Eᵥᵢᵢ | E₅ | E_{f} |
| 2167 | E₁ₐEᵢᵢE_{iib} | Eᵥᵢᵢ | E₅ | E_{f} |
| 2168 | E_{1b}EᵢᵢE_{iib} | Eᵥᵢᵢ | E₅ | E_{f} |
| 2169 | E₁ₐE₄ | Eᵥᵢᵢ | E₅ | E_{f} |
| 2170 | E_{1b}E₄ | Eᵥᵢᵢ | E₅ | E_{f} |
| 2171 | E₁ₐEᵢE₄ | Eᵥᵢᵢ | E₅ | E_{f} |
| 2172 | E_{1b}EᵢE₄ | Eᵥᵢᵢ | E₅ | E_{f} |
| 2173 | E₁ₐEᵢᵢE₄ | Eᵥᵢᵢ | E₅ | E_{f} |
| 2174 | E_{1b}EᵢᵢE₄ | Eᵥᵢᵢ | E₅ | E_{f} |
| 2175 | E₁ₐEᵢᵢE_{iib}E₄ | Eᵥᵢᵢ | E₅ | E_{f} |
| 2176 | E_{1b}EᵢᵢE_{iib}E₄ | Eᵥᵢᵢ | E₅ | E_{f} |
| 2177 | E₁ₐEᵢᵢᵢ | Eᵥᵢᵢ | E₅ | E_{f} |
| 2178 | E_{1b}Eᵢᵢᵢ | Eᵥᵢᵢ | E₅ | E_{f} |
| 2179 | E₁ₐEᵢᵢᵢE₄ | Eᵥᵢᵢ | E₅ | E_{f} |
| 2180 | E_{1b}EᵢᵢᵢE₄ | Eᵥᵢᵢ | E₅ | E_{f} |
| 2181 | E₁ₐEᵢᵥ | Eᵥᵢᵢ | E₅ | E_{f} |
| 2182 | E_{1b}Eᵢᵥ | Eᵥᵢᵢ | E₅ | E_{f} |
| 2183 | E₁ₐEᵢᵥE₄ | Eᵥᵢᵢ | E₅ | E_{f} |
| 2184 | E_{1b}EᵢᵥE₄ | Eᵥᵢᵢ | E₅ | E_{f} |
| 2185 | E₁ₐ | Eᵥᵢᵢ | E₄*E₅ | E_{f} |
| 2186 | E_{1b} | Eᵥᵢᵢ | E₄*E₅ | E_{f} |
| 2187 | E₁ₐEᵢ | Eᵥᵢᵢ | E₄*E₅ | E_{f} |
| 2188 | E_{1b}Eᵢ | Eᵥᵢᵢ | E₄*E₅ | E_{f} |
| 2189 | E₁ₐEᵢᵢ | Eᵥᵢᵢ | E₄*E₅ | E_{f} |
| 2190 | E_{1b}Eᵢᵢ | Eᵥᵢᵢ | E₄*E₅ | E_{f} |
| 2191 | E₁ₐEᵢᵢE_{iib} | Eᵥᵢᵢ | E₄*E₅ | E_{f} |
| 2192 | E_{1b}EᵢᵢE_{iib} | Eᵥᵢᵢ | E₄*E₅ | E_{f} |
| 2193 | E₁ₐE₄ | Eᵥᵢᵢ | E₄*E₅ | E_{f} |
| 2194 | E_{1b}E₄ | Eᵥᵢᵢ | E₄*E₅ | E_{f} |
| 2195 | E₁ₐEᵢE₄ | Eᵥᵢᵢ | E₄*E₅ | E_{f} |
| 2196 | E_{1b}EᵢE₄ | Eᵥᵢᵢ | E₄*E₅ | E_{f} |
| 2197 | E₁ₐEᵢᵢE₄ | Eᵥᵢᵢ | E₄*E₅ | E_{f} |
| 2198 | E_{1b}EᵢᵢE₄ | Eᵥᵢᵢ | E₄*E₅ | E_{f} |
| 2199 | E₁ₐEᵢᵢE_{iib}E₄ | Eᵥᵢᵢ | E₄*E₅ | E_{f} |
| 2200 | E_{1b}EᵢᵢE_{iib}E₄ | Eᵥᵢᵢ | E₄*E₅ | E_{f} |
| 2201 | E₁ₐEᵢᵢᵢ | Eᵥᵢᵢ | E₄*E₅ | E_{f} |
| 2202 | E_{1b}Eᵢᵢᵢ | Eᵥᵢᵢ | E₄*E₅ | E_{f} |
| 2203 | E₁ₐEᵢᵢᵢE₄ | Eᵥᵢᵢ | E₄*E₅ | E_{f} |
| 2204 | E_{1b}EᵢᵢᵢE₄ | Eᵥᵢᵢ | E₄*E₅ | E_{f} |
| 2205 | E₁ₐEᵢᵥ | Eᵥᵢᵢ | E₄*E₅ | E_{f} |
| 2206 | E_{1b}Eᵢᵥ | Eᵥᵢᵢ | E₄*E₅ | E_{f} |
| 2207 | E₁ₐEᵢᵥE₄ | Eᵥᵢᵢ | E₄*E₅ | E_{f} |
| 2208 | E_{1b}EᵢᵥE₄ | Eᵥᵢᵢ | E₄*E₅ | E_{f} |
| 2209 | E₁ₐ | AlkLEᵥᵢᵢ | | E_{f} |
| 2210 | E_{1b} | AlkLEᵥᵢᵢ | | E_{f} |
| 2211 | E₁ₐEᵢ | AlkLEᵥᵢᵢ | | E_{f} |
| 2212 | E_{1b}Eᵢ | AlkLEᵥᵢᵢ | | E_{f} |
| 2213 | E₁ₐEᵢᵢ | AlkLEᵥᵢᵢ | | E_{f} |
| 2214 | E_{1b}Eᵢᵢ | AlkLEᵥᵢᵢ | | E_{f} |
| 2215 | E₁ₐEᵢᵢE_{iib} | AlkLEᵥᵢᵢ | | E_{f} |
| 2216 | E_{1b}EᵢᵢE_{iib} | AlkLEᵥᵢᵢ | | E_{f} |
| 2217 | E₁ₐE₄ | AlkLEᵥᵢᵢ | | E_{f} |
| 2218 | E_{1b}E₄ | AlkLEᵥᵢᵢ | | E_{f} |
| 2219 | E₁ₐEᵢE₄ | AlkLEᵥᵢᵢ | | E_{f} |
| 2220 | E_{1b}EᵢE₄ | AlkLEᵥᵢᵢ | | E_{f} |
| 2221 | E₁ₐEᵢᵢE₄ | AlkLEᵥᵢᵢ | | E_{f} |
| 2222 | E_{1b}EᵢᵢE₄ | AlkLEᵥᵢᵢ | | E_{f} |
| 2223 | E₁ₐEᵢᵢE_{iib}E₄ | AlkLEᵥᵢᵢ | | E_{f} |
| 2224 | E_{1b}EᵢᵢE_{iib}E₄ | AlkLEᵥᵢᵢ | | E_{f} |
| 2225 | E₁ₐEᵢᵢᵢ | AlkLEᵥᵢᵢ | | E_{f} |
| 2226 | E_{1b}Eᵢᵢᵢ | AlkLEᵥᵢᵢ | | E_{f} |
| 2227 | E₁ₐEᵢᵢᵢE₄ | AlkLEᵥᵢᵢ | | E_{f} |
| 2228 | E_{1b}EᵢᵢᵢE₄ | AlkLEᵥᵢᵢ | | E_{f} |
| 2229 | E₁ₐEᵢᵥ | AlkLEᵥᵢᵢ | | E_{f} |
| 2230 | E_{1b}Eᵢᵥ | AlkLEᵥᵢᵢ | | E_{f} |
| 2231 | E₁ₐEᵢᵥE₄ | AlkLEᵥᵢᵢ | | E_{f} |
| 2232 | E_{1b}EᵢᵥE₄ | AlkLEᵥᵢᵢ | | E_{f} |
| 2233 | E₁ₐ | AlkLEᵥᵢᵢ | E₄* | E_{f} |
| 2234 | E_{1b} | AlkLEᵥᵢᵢ | E₄* | E_{f} |
| 2235 | E₁ₐEᵢ | AlkLEᵥᵢᵢ | E₄* | E_{f} |
| 2236 | E_{1b}Eᵢ | AlkLEᵥᵢᵢ | E₄* | E_{f} |
| 2237 | E₁ₐEᵢᵢ | AlkLEᵥᵢᵢ | E₄* | E_{f} |
| 2238 | E_{1b}Eᵢᵢ | AlkLEᵥᵢᵢ | E₄* | E_{f} |
| 2239 | E₁ₐEᵢᵢE_{iib} | AlkLEᵥᵢᵢ | E₄* | E_{f} |
| 2240 | E_{1b}EᵢᵢE_{iib} | AlkLEᵥᵢᵢ | E₄* | E_{f} |
| 2241 | E₁ₐE₄ | AlkLEᵥᵢᵢ | E₄* | E_{f} |
| 2242 | E_{1b}E₄ | AlkLEᵥᵢᵢ | E₄* | E_{f} |
| 2243 | E₁ₐEᵢE₄ | AlkLEᵥᵢᵢ | E₄* | E_{f} |
| 2244 | E_{1b}EᵢE₄ | AlkLEᵥᵢᵢ | E₄* | E_{f} |
| 2245 | E₁ₐEᵢᵢE₄ | AlkLEᵥᵢᵢ | E₄* | E_{f} |
| 2246 | E_{1b}EᵢᵢE₄ | AlkLEᵥᵢᵢ | E₄* | E_{f} |
| 2247 | E₁ₐEᵢᵢE_{iib}E₄ | AlkLEᵥᵢᵢ | E₄* | E_{f} |
| 2248 | E_{1b}EᵢᵢE_{iib}E₄ | AlkLEᵥᵢᵢ | E₄* | E_{f} |
| 2249 | E₁ₐEᵢᵢᵢ | AlkLEᵥᵢᵢ | E₄* | E_{f} |
| 2250 | E_{1b}Eᵢᵢᵢ | AlkLEᵥᵢᵢ | E₄* | E_{f} |
| 2251 | E₁ₐEᵢᵢᵢE₄ | AlkLEᵥᵢᵢ | E₄* | E_{f} |
| 2252 | E_{1b}EᵢᵢᵢE₄ | AlkLEᵥᵢᵢ | E₄* | E_{f} |
| 2253 | E₁ₐEᵢᵥ | AlkLEᵥᵢᵢ | E₄* | E_{f} |
| 2254 | E_{1b}Eᵢᵥ | AlkLEᵥᵢᵢ | E₄* | E_{f} |
| 2255 | E₁ₐEᵢᵥE₄ | AlkLEᵥᵢᵢ | E₄* | E_{f} |
| 2256 | E_{1b}EᵢᵥE₄ | AlkLEᵥᵢᵢ | E₄* | E_{f} |
| 2257 | E₁ₐ | AlkLEᵥᵢᵢ | E₅ | E_{f} |
| 2258 | E_{1b} | AlkLEᵥᵢᵢ | E₅ | E_{f} |
| 2259 | E₁ₐEᵢ | AlkLEᵥᵢᵢ | E₅ | E_{f} |
| 2260 | E_{1b}Eᵢ | AlkLEᵥᵢᵢ | E₅ | E_{f} |
| 2261 | E₁ₐEᵢᵢ | AlkLEᵥᵢᵢ | E₅ | E_{f} |
| 2262 | E_{1b}Eᵢᵢ | AlkLEᵥᵢᵢ | E₅ | E_{f} |
| 2263 | E₁ₐEᵢᵢE_{iib} | AlkLEᵥᵢᵢ | E₅ | E_{f} |
| 2264 | E_{1b}EᵢᵢE_{iib} | AlkLEᵥᵢᵢ | E₅ | E_{f} |
| 2265 | E₁ₐE₄ | AlkLEᵥᵢᵢ | E₅ | E_{f} |
| 2266 | E_{1b}E₄ | AlkLEᵥᵢᵢ | E₅ | E_{f} |
| 2267 | E₁ₐEᵢE₄ | AlkLEᵥᵢᵢ | E₅ | E_{f} |
| 2268 | E_{1b}EᵢE₄ | AlkLEᵥᵢᵢ | E₅ | E_{f} |
| 2269 | E₁ₐEᵢᵢE₄ | AlkLEᵥᵢᵢ | E₅ | E_{f} |
| 2270 | E_{1b}EᵢᵢE₄ | AlkLEᵥᵢᵢ | E₅ | E_{f} |
| 2271 | E₁ₐEᵢᵢE_{iib}E₄ | AlkLEᵥᵢᵢ | E₅ | E_{f} |
| 2272 | E_{1b}EᵢᵢE_{iib}E₄ | AlkLEᵥᵢᵢ | E₅ | E_{f} |
| 2273 | E₁ₐEᵢᵢᵢ | AlkLEᵥᵢᵢ | E₅ | E_{f} |
| 2274 | E_{1b}Eᵢᵢᵢ | AlkLEᵥᵢᵢ | E₅ | E_{f} |
| 2275 | E₁ₐEᵢᵢᵢE₄ | AlkLEᵥᵢᵢ | E₅ | E_{f} |
| 2276 | E_{1b}EᵢᵢᵢE₄ | AlkLEᵥᵢᵢ | E₅ | E_{f} |
| 2277 | E₁ₐEᵢᵥ | AlkLEᵥᵢᵢ | E₅ | E_{f} |
| 2278 | E_{1b}Eᵢᵥ | AlkLEᵥᵢᵢ | E₅ | E_{f} |
| 2279 | E₁ₐEᵢᵥE₄ | AlkLEᵥᵢᵢ | E₅ | E_{f} |
| 2280 | E_{1b}EᵢᵥE₄ | AlkLEᵥᵢᵢ | E₅ | E_{f} |
| 2281 | E₁ₐ | AlkLEᵥᵢᵢ | E₄*E₅ | E_{f} |
| 2282 | E_{1b} | AlkLEᵥᵢᵢ | E₄*E₅ | E_{f} |
| 2283 | E₁ₐEᵢ | AlkLEᵥᵢᵢ | E₄*E₅ | E_{f} |
| 2284 | E_{1b}Eᵢ | AlkLEᵥᵢᵢ | E₄*E₅ | E_{f} |
| 2285 | E₁ₐEᵢᵢ | AlkLEᵥᵢᵢ | E₄*E₅ | E_{f} |
| 2286 | E_{1b}Eᵢᵢ | AlkLEᵥᵢᵢ | E₄*E₅ | E_{f} |
| 2287 | E₁ₐEᵢᵢE_{iib} | AlkLEᵥᵢᵢ | E₄*E₅ | E_{f} |
| 2288 | E_{1b}EᵢᵢE_{iib} | AlkLEᵥᵢᵢ | E₄*E₅ | E_{f} |
| 2289 | E₁ₐE₄ | AlkLEᵥᵢᵢ | E₄*E₅ | E_{f} |
| 2290 | E_{1b}E₄ | AlkLEᵥᵢᵢ | E₄*E₅ | E_{f} |
| 2291 | E₁ₐEᵢE₄ | AlkLEᵥᵢᵢ | E₄*E₅ | E_{f} |
| 2292 | E_{1b}EᵢE₄ | AlkLEᵥᵢᵢ | E₄*E₅ | E_{f} |
| 2293 | E₁ₐEᵢᵢE₄ | AlkLEᵥᵢᵢ | E₄*E₅ | E_{f} |
| 2294 | E_{1b}EᵢᵢE₄ | AlkLEᵥᵢᵢ | E₄*E₅ | E_{f} |
| 2295 | E₁ₐEᵢᵢE_{iib}E₄ | AlkLEᵥᵢᵢ | E₄*E₅ | E_{f} |
| 2296 | E_{1b}EᵢᵢE_{iib}E₄ | AlkLEᵥᵢᵢ | E₄*E₅ | E_{f} |
| 2297 | E₁ₐEᵢᵢᵢ | AlkLEᵥᵢᵢ | E₄*E₅ | E_{f} |
| 2298 | E_{1b}Eᵢᵢᵢ | AlkLEᵥᵢᵢ | E₄*E₅ | E_{f} |
| 2299 | E₁ₐEᵢᵢᵢE₄ | AlkLEᵥᵢᵢ | E₄*E₅ | E_{f} |
| 2300 | E_{1b}EᵢᵢᵢE₄ | AlkLEᵥᵢᵢ | E₄*E₅ | E_{f} |
| 2301 | E₁ₐEᵢᵥ | AlkLEᵥᵢᵢ | E₄*E₅ | E_{f} |
| 2302 | E_{1b}Eᵢᵥ | AlkLEᵥᵢᵢ | E₄*E₅ | E_{f} |
| 2303 | E₁ₐEᵢᵥE₄ | AlkLEᵥᵢᵢ | E₄*E₅ | E_{f} |
| 2304 | E_{1b}EᵢᵥE₄ | AlkLEᵥᵢᵢ | E₄*E₅ | E_{f} |

Microorganisms quite especially preferred according to the invention (abbreviated as MO) are outstandingly suitable for the production of ω-carboxycarboxylic acids and have increased or decreased enzyme activities (abbreviated as E) described in the following table, where these can in addition advantageously be combined with an increased enzyme activity compared to the wild type of the microorganism, which is described for the 3-ketoacyl-ACP (Acyl Carrrier Protein) synthase III (EC 2.3.1.41), in particular that from plants, preferably that from plants the seeds whereof contain fatty acids with alkyl residues shorter than 14 C atoms, and particularly preferably that from plants of the genera Cuphea, Elaeis, Cocos, Umbellularia and Cinnamomum and gene products selected from AccA, AccB, AccC, AccD, AceE, AceF, Lpd, AcpP, FabA, FabB, FabD, FabF, FabG, FabH, Fabl, FabZ, PanD, PanK, UdhA, PntA or PntB.

Any combinations of at least two of these enzyme activities can advantageously be increased.

In addition, moreover, it can be advantageous if the microorganism is provided with a lower enzyme activity compared to the wild type of the microorganism, which is described for the gene products selected from TdcE, PflA, PflB, PflC, PflD, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, singly or in any combination.

| | | |
|---|---|---|
| MO | E increased | E decreased |

| | | | | |
|---|---|---|---|---|
| 1 | E₁ₐ | | | |
| 2 | E_{1b} | | | |
| 3 | E₁ₐE₅ | | | |
| 4 | E_{1b}E₅ | | | |
| 5 | E₁ₐEᵢ | | | |
| 6 | E_{1b}Eᵢ | | | |
| 7 | E₁ₐEᵢᵢ | | | |
| 8 | E_{1b}Eᵢᵢ | | | |
| 9 | E₁ₐEᵢᵢE_{iib} | | | |
| 10 | E_{1b}EᵢᵢE_{iib} | | | |
| 11 | E₁ₐE₅Eᵢ | | | |
| 12 | E_{1b}E₅Eᵢ | | | |
| 13 | E₁ₐE₅Eᵢᵢ | | | |
| 14 | E_{1b}E₅Eᵢᵢ | | | |
| 15 | E₁ₐE₅EᵢᵢE_{iib} | | | |
| 16 | E_{1b}E₅EᵢᵢE_{iib} | | | |
| 17 | E₁ₐE₄ | | | |
| 18 | E_{1b}E₄ | | | |
| 19 | E₁ₐE₅E₄ | | | |
| 20 | E_{1b}E₅E₄ | | | |
| 21 | E₁ₐEᵢE₄ | | | |
| 22 | E_{1b}EᵢE₄ | | | |
| 23 | E₁ₐEᵢᵢE₄ | | | |
| 24 | E_{1b}EᵢᵢE₄ | | | |
| 25 | E₁ₐEᵢᵢE_{iib}E₄ | | | |
| 26 | E_{1b}EᵢᵢE_{iib}E₄ | | | |
| 27 | E₁ₐE₅EᵢE₄ | | | |
| 28 | E_{1b}E₅EᵢE₄ | | | |
| 29 | E₁ₐE₅EᵢᵢE₄ | | | |
| 30 | E_{1b}E₅EᵢᵢE₄ | | | |
| 31 | E₁ₐE₅EᵢᵢE_{iib}E₄ | | | |
| 32 | E_{1b}E₅EᵢᵢE_{iib}E₄ | | | |
| 33 | E₁ₐEᵢᵢᵢ | | | |
| 34 | E_{1b}Eᵢᵢᵢ | | | |
| 35 | E₁ₐE₅Eᵢᵢᵢ | | | |
| 36 | E_{1b}E₅Eᵢᵢᵢ | | | |
| 37 | E₁ₐEᵢᵢᵢE₄ | | | |
| 38 | E_{1b}EᵢᵢᵢE₄ | | | |
| 39 | E₁ₐE₅EᵢᵢᵢE₄ | | | |
| 40 | E_{1b}E₅EᵢᵢᵢE₄ | | | |
| 41 | E₁ₐEᵢᵥ | | | |
| 42 | E_{1b}Eᵢᵥ | | | |
| 43 | E₁ₐE₅Eᵢᵥ | | | |
| 44 | E_{1b}E₅Eᵢᵥ | | | |
| 45 | E₁ₐEᵢᵥE₄ | | | |
| 46 | E_{1b}EᵢᵥE₄ | | | |
| 47 | E₁ₐE₅EᵢᵥE₄ | | | |
| 48 | E_{1b}E₅EᵢᵥE₄ | | | |
| 49 | E₁ₐ | | E₄* | |
| 50 | E_{1b} | | E₄* | |
| 51 | E₁ₐE₅ | | E₄* | |
| 52 | E_{1b}E₅ | | E₄* | |
| 53 | E₁ₐEᵢ | | E₄* | |
| 54 | E_{1b}Eᵢ | | E₄* | |
| 55 | E₁ₐEᵢᵢ | | E₄* | |
| 56 | E_{1b}Eᵢᵢ | | E₄* | |
| 57 | E₁ₐEᵢᵢE_{iib} | | E₄* | |
| 58 | E_{1b}EᵢᵢE_{iib} | | E₄* | |
| 59 | E₁ₐE₅Eᵢ | | E₄* | |
| 60 | E_{1b}E₅Eᵢ | | E₄* | |
| 61 | E₁ₐE₅Eᵢᵢ | | E₄* | |
| 62 | E_{1b}E₅Eᵢᵢ | | E₄* | |
| 63 | E₁ₐE₅EᵢᵢE_{iib} | | E₄* | |
| 64 | E_{1b}E₅EᵢᵢE_{iib} | | E₄* | |
| 65 | E₁ₐE₄ | | E₄* | |
| 66 | E_{1b}E₄ | | E₄* | |
| 67 | E₁ₐE₅E₄ | | E₄* | |
| 68 | E_{1b}E₅E₄ | | E₄* | |
| 69 | E₁ₐEᵢE₄ | | E₄* | |
| 70 | E_{1b}EᵢE₄ | | E₄* | |
| 71 | E₁ₐEᵢᵢE₄ | | E₄* | |
| 72 | E_{1b}EᵢᵢE₄ | | E₄* | |
| 73 | E₁ₐEᵢᵢE_{iib}E₄ | | E₄* | |
| 74 | E_{1b}EᵢᵢE_{iib}E₄ | | E₄* | |
| 75 | E₁ₐE₅EᵢE₄ | | E₄* | |
| 76 | E_{1b}E₅EᵢE₄ | | E₄* | |
| 77 | E₁ₐE₅EᵢᵢE₄ | | E₄* | |
| 78 | E_{1b}E₅EᵢᵢE₄ | | E₄* | |
| 79 | E₁ₐE₅EᵢᵢE_{iib}E₄ | | E₄* | |
| 80 | E_{1b}E₅EᵢᵢE_{iib}E₄ | | E₄* | |
| 81 | E₁ₐEᵢᵢᵢ | | E₄* | |
| 82 | E_{1b}Eᵢᵢᵢ | | E₄* | |
| 83 | E₁ₐE₅Eᵢᵢᵢ | | E₄* | |
| 84 | E_{1b}E₅Eᵢᵢᵢ | | E₄* | |
| 85 | E₁ₐEᵢᵢᵢE₄ | | E₄* | |
| 86 | E_{1b}EᵢᵢᵢE₄ | | E₄* | |
| 87 | E₁ₐE₅EᵢᵢᵢE₄ | | E₄* | |
| 88 | E_{1b}E₅EᵢᵢᵢE₄ | | E₄* | |
| 89 | E₁ₐEᵢᵥ | | E₄* | |
| 90 | E_{1b}Eᵢᵥ | | E₄* | |
| 91 | E₁ₐE₅Eᵢᵥ | | E₄* | |
| 92 | E_{1b}E₅Eᵢᵥ | | E₄* | |
| 93 | E₁ₐEᵢᵥE₄ | | E₄* | |
| 94 | E_{1b}EᵢᵥE₄ | | E₄* | |
| 95 | E₁ₐE₅EᵢᵥE₄ | | E₄* | |
| 96 | E_{1b}E₅EᵢᵥE₄ | | E₄* | |
| 97 | E₁ₐ | AlkL | | |
| 98 | E_{1b} | AlkL | | |
| 99 | E₁ₐE₅ | AlkL | | |
| 100 | E_{1b}E₅ | AlkL | | |
| 101 | E₁ₐEᵢ | AlkL | | |
| 102 | E_{1b}Eᵢ | AlkL | | |
| 103 | E₁ₐEᵢᵢ | AlkL | | |
| 104 | E_{1b}Eᵢᵢ | AlkL | | |
| 105 | E₁ₐEᵢᵢE_{iib} | AlkL | | |
| 106 | E_{1b}EᵢᵢE_{iib} | AlkL | | |
| 107 | E₁ₐE₅Eᵢ | AlkL | | |
| 108 | E_{1b}E₅Eᵢ | AlkL | | |
| 109 | E₁ₐE₅Eᵢᵢ | AlkL | | |
| 110 | E_{1b}E₅Eᵢᵢ | AlkL | | |
| 111 | E₁ₐE₅EᵢᵢE_{iib} | AlkL | | |
| 112 | E_{1b}E₅EᵢᵢE_{iib} | AlkL | | |
| 113 | E₁ₐE₄ | AlkL | | |
| 114 | E_{1b}E₄ | AlkL | | |
| 115 | E₁ₐE₅E₄ | AlkL | | |
| 116 | E_{1b}E₅E₄ | AlkL | | |
| 117 | E₁ₐEᵢE₄ | AlkL | | |
| 118 | E_{1b}EᵢE₄ | AlkL | | |
| 119 | E₁ₐEᵢᵢE₄ | AlkL | | |
| 120 | E_{1b}EᵢᵢE₄ | AlkL | | |
| 121 | E₁ₐEᵢᵢE_{iib}E₄ | AlkL | | |
| 122 | E_{1b}EᵢᵢE_{iib}E₄ | AlkL | | |
| 123 | E₁ₐE₅EᵢE₄ | AlkL | | |
| 124 | E_{1b}E₅EᵢE₄ | AlkL | | |
| 125 | E₁ₐE₅EᵢᵢE₄ | AlkL | | |
| 126 | E_{1b}E₅EᵢᵢE₄ | AlkL | | |
| 127 | E₁ₐE₅EᵢᵢE_{iib}E₄ | AlkL | | |
| 128 | E_{1b}E₅EᵢᵢE_{iib}E₄ | AlkL | | |
| 129 | E₁ₐEᵢᵢᵢ | AlkL | | |
| 130 | E_{1b}Eᵢᵢᵢ | AlkL | | |
| 131 | E₁ₐE₅Eᵢᵢᵢ | AlkL | | |
| 132 | E_{1b}E₅Eᵢᵢᵢ | AlkL | | |
| 133 | E₁ₐEᵢᵢᵢE₄ | AlkL | | |
| 134 | E_{1b}EᵢᵢᵢE₄ | AlkL | | |
| 135 | E₁ₐE₅EᵢᵢᵢE₄ | AlkL | | |
| 136 | E_{1b}E₅EᵢᵢᵢE₄ | AlkL | | |
| 137 | E₁ₐEᵢᵥ | AlkL | | |
| 138 | E_{1b}Eᵢᵥ | AlkL | | |
| 139 | E₁ₐE₅Eᵢᵥ | AlkL | | |
| 140 | E_{1b}E₅Eᵢᵥ | AlkL | | |
| 141 | E₁ₐEᵢᵥE₄ | AlkL | | |
| 142 | E_{1b}EᵢᵥE₄ | AlkL | | |
| 143 | E₁ₐE₅EᵢᵥE₄ | AlkL | | |
| 144 | E_{1b}E₅EᵢᵥE₄ | AlkL | | |
| 145 | E₁ₐ | AlkL | E₄* | |
| 146 | E_{1b} | AlkL | E₄* | |
| 147 | E₁ₐE₅ | AlkL | E₄* | |
| 148 | E_{1b}E₅ | AlkL | E₄* | |
| 149 | E₁ₐEᵢ | AlkL | E₄* | |
| 150 | E_{1b}Eᵢ | AlkL | E₄* | |
| 151 | E₁ₐEᵢᵢ | AlkL | E₄* | |
| 152 | E_{1b}Eᵢᵢ | AlkL | E₄* | |
| 153 | E₁ₐEᵢᵢE_{iib} | AlkL | E₄* | |
| 154 | E_{1b}EᵢᵢE_{iib} | AlkL | E₄* | |
| 155 | E₁ₐE₅Eᵢ | AlkL | E₄* | |
| 156 | E_{1b}E₅Eᵢ | AlkL | E₄* | |
| 157 | E₁ₐE₅Eᵢᵢ | AlkL | E₄* | |
| 158 | E_{1b}E₅Eᵢᵢ | AlkL | E₄* | |
| 159 | E₁ₐE₅EᵢᵢE_{iib} | AlkL | E₄* | |
| 160 | E_{1b}E₅EᵢᵢE_{iib} | AlkL | E₄* | |
| 161 | E₁ₐE₄ | AlkL | E₄* | |
| 162 | E_{1b}E₄ | AlkL | E₄* | |
| 163 | E₁ₐE₅E₄ | AlkL | E₄* | |
| 164 | E_{1b}E₅E₄ | AlkL | E₄* | |
| 165 | E₁ₐEᵢE₄ | AlkL | E₄* | |
| 166 | E_{1b}EᵢE₄ | AlkL | E₄* | |
| 167 | E₁ₐEᵢᵢE₄ | AlkL | E₄* | |
| 168 | E_{1b}EᵢᵢE₄ | AlkL | E₄* | |
| 169 | E₁ₐEᵢᵢE_{iib}E₄ | AlkL | E₄* | |
| 170 | E_{1b}EᵢᵢE_{iib}E₄ | AlkL | E₄* | |
| 171 | E₁ₐE₅EᵢE₄ | AlkL | E₄* | |
| 172 | E_{1b}E₅EᵢE₄ | AlkL | E₄* | |
| 173 | E₁ₐE₅EᵢᵢE₄ | AlkL | E₄* | |
| 174 | E_{1b}E₅EᵢᵢE₄ | AlkL | E₄* | |
| 175 | E₁ₐE₅EᵢᵢE_{iib}E₄ | AlkL | E₄* | |
| 176 | E_{1b}E₅EᵢᵢE_{iib}E₄ | AlkL | E₄* | |
| 177 | E₁ₐEᵢᵢᵢ | AlkL | E₄* | |
| 178 | E_{1b}Eᵢᵢᵢ | AlkL | E₄* | |
| 179 | E₁ₐE₅Eᵢᵢᵢ | AlkL | E₄* | |
| 180 | E_{1b}E₅Eᵢᵢᵢ | AlkL | E₄* | |
| 181 | E₁ₐEᵢᵢᵢE₄ | AlkL | E₄* | |
| 182 | E_{1b}EᵢᵢᵢE₄ | AlkL | E₄* | |
| 183 | E₁ₐE₅EᵢᵢᵢE₄ | AlkL | E₄* | |
| 184 | E_{1b}E₅EᵢᵢᵢE₄ | AlkL | E₄* | |
| 185 | E₁ₐEᵢᵥ | AlkL | E₄* | |
| 186 | E_{1b}Eᵢᵥ | AlkL | E₄* | |
| 187 | E₁ₐE₅Eᵢᵥ | AlkL | E₄* | |
| 188 | E_{1b}E₅Eᵢᵥ | AlkL | E₄* | |
| 189 | E₁ₐEᵢᵥE₄ | AlkL | E₄* | |
| 190 | E_{1b}EᵢᵥE₄ | AlkL | E₄* | |
| 191 | E₁ₐE₅EᵢᵥE₄ | AlkL | E₄* | |
| 192 | E_{1b}E₅EᵢᵥE₄ | AlkL | E₄* | |
| 193 | E₁ₐ | | | Eₐ |
| 194 | E_{1b} | | | Eₐ |
| 195 | E₁ₐE₅ | | | Eₐ |
| 196 | E_{1b}E₅ | | | Eₐ |
| 197 | E₁ₐEᵢ | | | Eₐ |
| 198 | E_{1b}Eᵢ | | | Eₐ |
| 199 | E₁ₐEᵢᵢ | | | Eₐ |
| 200 | E_{1b}Eᵢᵢ | | | Eₐ |
| 201 | E₁ₐEᵢᵢE_{iib} | | | Eₐ |
| 202 | E_{1b}EᵢᵢE_{iib} | | | Eₐ |
| 203 | E₁ₐE₅Eᵢ | | | Eₐ |
| 204 | E_{1b}E₅Eᵢ | | | Eₐ |
| 205 | E₁ₐE₅Eᵢᵢ | | | Eₐ |
| 206 | E_{1b}E₅Eᵢᵢ | | | Eₐ |
| 207 | E₁ₐE₅EᵢᵢE_{iib} | | | Eₐ |
| 208 | E_{1b}E₅EᵢᵢE_{iib} | | | Eₐ |
| 209 | E₁ₐE₄ | | | Eₐ |
| 210 | E_{1b}E₄ | | | Eₐ |
| 211 | E₁ₐE₅E₄ | | | Eₐ |
| 212 | E_{1b}E₅E₄ | | | Eₐ |
| 213 | E₁ₐEᵢE₄ | | | Eₐ |
| 214 | E_{1b}EᵢE₄ | | | Eₐ |
| 215 | E₁ₐEᵢᵢE₄ | | | Eₐ |
| 216 | E_{1b}EᵢᵢE₄ | | | Eₐ |
| 217 | E₁ₐEᵢᵢE_{iib}E₄ | | | Eₐ |
| 218 | E_{1b}EᵢᵢE_{iib}E₄ | | | Eₐ |
| 219 | E₁ₐE₅EᵢE₄ | | | Eₐ |
| 220 | E_{1b}E₅EᵢE₄ | | | Eₐ |
| 221 | E₁ₐE₅EᵢᵢE₄ | | | Eₐ |
| 222 | E_{1b}E₅EᵢᵢE₄ | | | Eₐ |
| 223 | E₁ₐE₅EᵢᵢE_{iib}E₄ | | | Eₐ |
| 224 | E_{1b}E₅EᵢᵢE_{iib}E₄ | | | Eₐ |
| 225 | E₁ₐEᵢᵢᵢ | | | Eₐ |
| 226 | E_{1b}Eᵢᵢᵢ | | | Eₐ |
| 227 | E₁ₐE₅Eᵢᵢᵢ | | | Eₐ |
| 228 | E_{1b}E₅Eᵢᵢᵢ | | | Eₐ |
| 229 | E₁ₐEᵢᵢᵢE₄ | | | Eₐ |
| 230 | E_{1b}EᵢᵢᵢE₄ | | | Eₐ |
| 231 | E₁ₐE₅EᵢᵢᵢE₄ | | | Eₐ |
| 232 | E_{1b}E₅EᵢᵢᵢE₄ | | | Eₐ |
| 233 | E₁ₐEᵢᵥ | | | Eₐ |
| 234 | E_{1b}Eᵢᵥ | | | Eₐ |
| 235 | E₁ₐE₅Eᵢᵥ | | | Eₐ |
| 236 | E_{1b}E₅Eᵢᵥ | | | Eₐ |
| 237 | E₁ₐEᵢᵥE₄ | | | Eₐ |
| 238 | E_{1b}EᵢᵥE₄ | | | Eₐ |
| 239 | E₁ₐE₅EᵢᵥE₄ | | | Eₐ |
| 240 | E_{1b}E₅EᵢᵥE₄ | | | Eₐ |
| 241 | E₁ₐ | | E₄* | Eₐ |
| 242 | E_{1b} | | E₄* | Eₐ |
| 243 | E₁ₐE₅ | | E₄* | Eₐ |
| 244 | E_{1b}E₅ | | E₄* | Eₐ |
| 245 | E₁ₐEᵢ | | E₄* | Eₐ |
| 246 | E_{1b}Eᵢ | | E₄* | Eₐ |
| 247 | E₁ₐEᵢᵢ | | E₄* | Eₐ |
| 248 | E_{1b}Eᵢᵢ | | E₄* | Eₐ |
| 249 | E₁ₐEᵢᵢE_{iib} | | E₄* | Eₐ |
| 250 | E_{1b}EᵢᵢE_{iib} | | E₄* | Eₐ |
| 251 | E₁ₐE₅Eᵢ | | E₄* | Eₐ |
| 252 | E_{1b}E₅Eᵢ | | E₄* | Eₐ |
| 253 | E₁ₐE₅Eᵢᵢ | | E₄* | Eₐ |
| 254 | E_{1b}E₅Eᵢᵢ | | E₄* | Eₐ |
| 255 | E₁ₐE₅EᵢᵢE_{iib} | | E₄* | Eₐ |
| 256 | E_{1b}E₅EᵢᵢE_{iib} | | E₄* | Eₐ |
| 257 | E₁ₐE₄ | | E₄* | Eₐ |
| 258 | E_{1b}E₄ | | E₄* | Eₐ |
| 259 | E₁ₐE₅E₄ | | E₄* | Eₐ |
| 260 | E_{1b}E₅E₄ | | E₄* | Eₐ |
| 261 | E₁ₐEᵢE₄ | | E₄* | Eₐ |
| 262 | E_{1b}EᵢE₄ | | E₄* | Eₐ |
| 263 | E₁ₐEᵢᵢE₄ | | E₄* | Eₐ |
| 264 | E_{1b}EᵢᵢE₄ | | E₄* | Eₐ |
| 265 | E₁ₐEᵢᵢE_{iib}E₄ | | E₄* | Eₐ |
| 266 | E_{1b}EᵢᵢE_{iib}E₄ | | E₄* | Eₐ |
| 267 | E₁ₐE₅EᵢE₄ | | E₄* | Eₐ |
| 268 | E_{1b}E₅EᵢE₄ | | E₄* | Eₐ |
| 269 | E₁ₐE₅EᵢᵢE₄ | | E₄* | Eₐ |
| 270 | E_{1b}E₅EᵢᵢE₄ | | E₄* | Eₐ |
| 271 | E₁ₐE₅EᵢᵢE_{iib}E₄ | | E₄* | Eₐ |
| 272 | E_{1b}E₅EᵢᵢE_{iib}E₄ | | E₄* | Eₐ |
| 273 | E₁ₐEᵢᵢᵢ | | E₄* | Eₐ |
| 274 | E_{1b}Eᵢᵢᵢ | | E₄* | Eₐ |
| 275 | E₁ₐE₅Eᵢᵢᵢ | | E₄* | Eₐ |
| 276 | E_{1b}E₅Eᵢᵢᵢ | | E₄* | Eₐ |
| 277 | E₁ₐEᵢᵢᵢE₄ | | E₄* | Eₐ |
| 278 | E_{1b}EᵢᵢᵢE₄ | | E₄* | Eₐ |
| 279 | E₁ₐE₅EᵢᵢᵢE₄ | | E₄* | Eₐ |
| 280 | E_{1b}E₅EᵢᵢᵢE₄ | | E₄* | Eₐ |
| 281 | E₁ₐEᵢᵥ | | E₄* | Eₐ |
| 282 | E_{1b}Eᵢᵥ | | E₄* | Eₐ |
| 283 | E₁ₐE₅Eᵢᵥ | | E₄* | Eₐ |
| 284 | E_{1b}E₅Eᵢᵥ | | E₄* | Eₐ |
| 285 | E₁ₐEᵢᵥE₄ | | E₄* | Eₐ |
| 286 | E_{1b}EᵢᵥE₄ | | E₄* | Eₐ |
| 287 | E₁ₐE₅EᵢᵥE₄ | | E₄* | Eₐ |
| 288 | E_{1b}E₅EᵢᵥE₄ | | E₄* | Eₐ |
| 289 | E₁ₐ | AlkL | | Eₐ |
| 290 | E_{1b} | AlkL | | Eₐ |
| 291 | E₁ₐE₅ | AlkL | | Eₐ |
| 292 | E_{1b}E₅ | AlkL | | Eₐ |
| 293 | E₁ₐEᵢ | AlkL | | Eₐ |
| 294 | E_{1b}Eᵢ | AlkL | | Eₐ |
| 295 | E₁ₐEᵢᵢ | AlkL | | Eₐ |
| 296 | E_{1b}Eᵢᵢ | AlkL | | Eₐ |
| 297 | E₁ₐEᵢᵢE_{iib} | AlkL | | Eₐ |
| 298 | E_{1b}EᵢᵢE_{iib} | AlkL | | Eₐ |
| 299 | E₁ₐE₅Eᵢ | AlkL | | Eₐ |
| 300 | E_{1b}E₅Eᵢ | AlkL | | Eₐ |
| 301 | E₁ₐE₅Eᵢᵢ | AlkL | | Eₐ |
| 302 | E_{1b}E₅Eᵢᵢ | AlkL | | Eₐ |
| 303 | E₁ₐE₅EᵢᵢE_{iib} | AlkL | | Eₐ |
| 304 | E_{1b}E₅EᵢᵢE_{iib} | AlkL | | Eₐ |
| 305 | E₁ₐE₄ | AlkL | | Eₐ |
| 306 | E_{1b}E₄ | AlkL | | Eₐ |
| 307 | E₁ₐE₅E₄ | AlkL | | Eₐ |
| 308 | E_{1b}E₅E₄ | AlkL | | Eₐ |
| 309 | E₁ₐEᵢE₄ | AlkL | | Eₐ |
| 310 | E_{1b}EᵢE₄ | AlkL | | Eₐ |
| 311 | E₁ₐEᵢᵢE₄ | AlkL | | Eₐ |
| 312 | E_{1b}EᵢᵢE₄ | AlkL | | Eₐ |
| 313 | E₁ₐEᵢᵢE_{iib}E₄ | AlkL | | Eₐ |
| 314 | E_{1b}EᵢᵢE_{iib}E₄ | AlkL | | Eₐ |
| 315 | E₁ₐE₅EᵢE₄ | AlkL | | Eₐ |
| 316 | E_{1b}E₅EᵢE₄ | AlkL | | Eₐ |
| 317 | E₁ₐE₅EᵢᵢE₄ | AlkL | | Eₐ |
| 318 | E_{1b}E₅EᵢᵢE₄ | AlkL | | Eₐ |
| 319 | E₁ₐE₅EᵢᵢE_{iib}E₄ | AlkL | | Eₐ |
| 320 | E_{1b}E₅EᵢᵢE_{iib}E₄ | AlkL | | Eₐ |
| 321 | E₁ₐEᵢᵢᵢ | AlkL | | Eₐ |
| 322 | E_{1b}Eᵢᵢᵢ | AlkL | | Eₐ |
| 323 | E₁ₐE₅Eᵢᵢᵢ | AlkL | | Eₐ |
| 324 | E_{1b}E₅Eᵢᵢᵢ | AlkL | | Eₐ |
| 325 | E₁ₐEᵢᵢᵢE₄ | AlkL | | Eₐ |
| 326 | E_{1b}EᵢᵢᵢE₄ | AlkL | | Eₐ |
| 327 | E₁ₐE₅EᵢᵢᵢE₄ | AlkL | | Eₐ |
| 328 | E_{1b}E₅EᵢᵢᵢE₄ | AlkL | | Eₐ |
| 329 | E₁ₐEᵢᵥ | AlkL | | Eₐ |
| 330 | E_{1b}Eᵢᵥ | AlkL | | Eₐ |
| 331 | E₁ₐE₅Eᵢᵥ | AlkL | | Eₐ |
| 332 | E_{1b}E₅Eᵢᵥ | AlkL | | Eₐ |
| 333 | E₁ₐEᵢᵥE₄ | AlkL | | Eₐ |
| 334 | E_{1b}EᵢᵥE₄ | AlkL | | Eₐ |
| 335 | E₁ₐE₅EᵢᵥE₄ | AlkL | | Eₐ |
| 336 | E_{1b}E₅EᵢᵥE₄ | AlkL | | Eₐ |
| 337 | E₁ₐ | AlkL | E₄* | Eₐ |
| 338 | E_{1b} | AlkL | E₄* | Eₐ |
| 339 | E₁ₐE₅ | AlkL | E₄* | Eₐ |
| 340 | E_{1b}E₅ | AlkL | E₄* | Eₐ |
| 341 | E₁ₐEᵢ | AlkL | E₄* | Eₐ |
| 342 | E_{1b}Eᵢ | AlkL | E₄* | Eₐ |
| 343 | E₁ₐEᵢᵢ | AlkL | E₄* | Eₐ |
| 344 | E_{1b}Eᵢᵢ | AlkL | E₄* | Eₐ |
| 345 | E₁ₐEᵢᵢE_{iib} | AlkL | E₄* | Eₐ |
| 346 | E_{1b}EᵢᵢE_{iib} | AlkL | E₄* | Eₐ |
| 347 | E₁ₐE₅Eᵢ | AlkL | E₄* | Eₐ |
| 348 | E_{1b}E₅Eᵢ | AlkL | E₄* | Eₐ |
| 349 | E₁ₐE₅Eᵢᵢ | AlkL | E₄* | Eₐ |
| 350 | E_{1b}E₅Eᵢᵢ | AlkL | E₄* | Eₐ |
| 351 | E₁ₐE₅EᵢᵢE_{iib} | AlkL | E₄* | Eₐ |
| 352 | E_{1b}E₅EᵢᵢE_{iib} | AlkL | E₄* | Eₐ |
| 353 | E₁ₐE₄ | AlkL | E₄* | Eₐ |
| 354 | E_{1b}E₄ | AlkL | E₄* | Eₐ |
| 355 | E₁ₐE₅E₄ | AlkL | E₄* | Eₐ |
| 356 | E_{1b}E₅E₄ | AlkL | E₄* | Eₐ |
| 357 | E₁ₐEᵢE₄ | AlkL | E₄* | Eₐ |
| 358 | E_{1b}EᵢE₄ | AlkL | E₄* | Eₐ |
| 359 | E₁ₐEᵢᵢE₄ | AlkL | E₄* | Eₐ |
| 360 | E_{1b}EᵢᵢE₄ | AlkL | E₄* | Eₐ |
| 361 | E₁ₐEᵢᵢE_{iib}E₄ | AlkL | E₄* | Eₐ |
| 362 | E_{1b}EᵢᵢE_{iib}E₄ | AlkL | E₄* | Eₐ |
| 363 | E₁ₐE₅EᵢE₄ | AlkL | E₄* | Eₐ |
| 364 | E_{1b}E₅EᵢE₄ | AlkL | E₄* | Eₐ |
| 365 | E₁ₐE₅EᵢᵢE₄ | AlkL | E₄* | Eₐ |
| 366 | E_{1b}E₅EᵢᵢE₄ | AlkL | E₄* | Eₐ |
| 367 | E₁ₐE₅EᵢᵢE_{iib}E₄ | AlkL | E₄* | Eₐ |
| 368 | E_{1b}E₅EᵢᵢE_{iib}E₄ | AlkL | E₄* | Eₐ |
| 369 | E₁ₐEᵢᵢᵢ | AlkL | E₄* | Eₐ |
| 370 | E_{1b}Eᵢᵢᵢ | AlkL | E₄* | Eₐ |
| 371 | E₁ₐE₅Eᵢᵢᵢ | AlkL | E₄* | Eₐ |
| 372 | E_{1b}E₅Eᵢᵢᵢ | AlkL | E₄* | Eₐ |
| 373 | E₁ₐEᵢᵢᵢE₄ | AlkL | E₄* | Eₐ |
| 374 | E_{1b}EᵢᵢᵢE₄ | AlkL | E₄* | Eₐ |
| 375 | E₁ₐE₅EᵢᵢᵢE₄ | AlkL | E₄* | Eₐ |
| 376 | E_{1b}E₅EᵢᵢᵢE₄ | AlkL | E₄* | Eₐ |
| 377 | E₁ₐEᵢᵥ | AlkL | E₄* | Eₐ |
| 378 | E_{1b}Eᵢᵥ | AlkL | E₄* | Eₐ |
| 379 | E₁ₐE₅Eᵢᵥ | AlkL | E₄* | Eₐ |
| 380 | E_{1b}E₅Eᵢᵥ | AlkL | E₄* | Eₐ |
| 381 | E₁ₐEᵢᵥE₄ | AlkL | E₄* | Eₐ |
| 382 | E_{1b}EᵢᵥE₄ | AlkL | E₄* | Eₐ |
| 383 | E₁ₐE₅EᵢᵥE₄ | AlkL | E₄* | Eₐ |
| 384 | E_{1b}E₅EᵢᵥE₄ | AlkL | E₄* | Eₐ |
| 385 | E₁ₐ | | | E_{b} |
| 386 | E_{1b} | | | E_{b} |
| 387 | E₁ₐE₅ | | | E_{b} |
| 388 | E_{1b}E₅ | | | E_{b} |
| 389 | E₁ₐEᵢ | | | E_{b} |
| 390 | E_{1b}Eᵢ | | | E_{b} |
| 391 | E₁ₐEᵢᵢ | | | E_{b} |
| 392 | E_{1b}Eᵢᵢ | | | E_{b} |
| 393 | E₁ₐEᵢᵢE_{iib} | | | E_{b} |
| 394 | E_{1b}EᵢᵢE_{iib} | | | E_{b} |
| 395 | E₁ₐE₅Eᵢ | | | E_{b} |
| 396 | E_{1b}E₅Eᵢ | | | E_{b} |
| 397 | E₁ₐE₅Eᵢᵢ | | | E_{b} |
| 398 | E_{1b}E₅Eᵢᵢ | | | E_{b} |
| 399 | E₁ₐE₅EᵢᵢE_{iib} | | | E_{b} |
| 400 | E_{1b}E₅EᵢᵢE_{iib} | | | E_{b} |
| 401 | E₁ₐE₄ | | | E_{b} |
| 402 | E_{1b}E₄ | | | E_{b} |
| 403 | E₁ₐE₅E₄ | | | E_{b} |
| 404 | E_{1b}E₅E₄ | | | E_{b} |
| 405 | E₁ₐEᵢE₄ | | | E_{b} |
| 406 | E_{1b}EᵢE₄ | | | E_{b} |
| 407 | E₁ₐEᵢᵢE₄ | | | E_{b} |
| 408 | E_{1b}EᵢᵢE₄ | | | E_{b} |
| 409 | E₁ₐEᵢᵢE_{iib}E₄ | | | E_{b} |
| 410 | E_{1b}EᵢᵢE_{iib}E₄ | | | E_{b} |
| 411 | E₁ₐE₅EᵢE₄ | | | E_{b} |
| 412 | E_{1b}E₅EᵢE₄ | | | E_{b} |
| 413 | E₁ₐE₅EᵢᵢE₄ | | | E_{b} |
| 414 | E_{1b}E₅EᵢᵢE₄ | | | E_{b} |
| 415 | E₁ₐE₅EᵢᵢE_{iib}E₄ | | | E_{b} |
| 416 | E_{1b}E₅EᵢᵢE_{iib}E₄ | | | E_{b} |
| 417 | E₁ₐEᵢᵢᵢ | | | E_{b} |
| 418 | E_{1b}Eᵢᵢᵢ | | | E_{b} |
| 419 | E₁ₐE₅Eᵢᵢᵢ | | | E_{b} |
| 420 | E_{1b}E₅Eᵢᵢᵢ | | | E_{b} |
| 421 | E₁ₐEᵢᵢᵢE₄ | | | E_{b} |
| 422 | E_{1b}EᵢᵢᵢE₄ | | | E_{b} |
| 423 | E₁ₐE₅EᵢᵢᵢE₄ | | | E_{b} |
| 424 | E_{1b}E₅EᵢᵢᵢE₄ | | | E_{b} |
| 425 | E₁ₐEᵢᵥ | | | E_{b} |
| 426 | E_{1b}Eᵢᵥ | | | E_{b} |
| 427 | E₁ₐE₅Eᵢᵥ | | | E_{b} |
| 428 | E_{1b}E₅Eᵢᵥ | | | E_{b} |
| 429 | E₁ₐEᵢᵥE₄ | | | E_{b} |
| 430 | E_{1b}EᵢᵥE₄ | | | E_{b} |
| 431 | E₁ₐE₅EᵢᵥE₄ | | | E_{b} |
| 432 | E_{1b}E₅EᵢᵥE₄ | | | E_{b} |
| 433 | E₁ₐ | | E₄* | E_{b} |
| 434 | E_{1b} | | E₄* | E_{b} |
| 435 | E₁ₐE₅ | | E₄* | E_{b} |
| 436 | E_{1b}E₅ | | E₄* | E_{b} |
| 437 | E₁ₐEᵢ | | E₄* | E_{b} |
| 438 | E_{1b}Eᵢ | | E₄* | E_{b} |
| 439 | E₁ₐEᵢᵢ | | E₄* | E_{b} |
| 440 | E_{1b}Eᵢᵢ | | E₄* | E_{b} |
| 441 | E₁ₐEᵢᵢE_{iib} | | E₄* | E_{b} |
| 442 | E_{1b}EᵢᵢE_{iib} | | E₄* | E_{b} |
| 443 | E₁ₐE₅Eᵢ | | E₄* | E_{b} |
| 444 | E_{1b}E₅Eᵢ | | E₄* | E_{b} |
| 445 | E₁ₐE₅Eᵢᵢ | | E₄* | E_{b} |
| 446 | E_{1b}E₅Eᵢᵢ | | E₄* | E_{b} |
| 447 | E₁ₐE₅EᵢᵢE_{iib} | | E₄* | E_{b} |
| 448 | E_{1b}E₅EᵢᵢE_{iib} | | E₄* | E_{b} |
| 449 | E₁ₐE₄ | | E₄* | E_{b} |
| 450 | E_{1b}E₄ | | E₄* | E_{b} |
| 451 | E₁ₐE₅E₄ | | E₄* | E_{b} |
| 452 | E_{1b}E₅E₄ | | E₄* | E_{b} |
| 453 | E₁ₐEᵢE₄ | | E₄* | E_{b} |
| 454 | E_{1b}EᵢE₄ | | E₄* | E_{b} |
| 455 | E₁ₐEᵢᵢE₄ | | E₄* | E_{b} |
| 456 | E_{1b}EᵢᵢE₄ | | E₄* | E_{b} |
| 457 | E₁ₐEᵢᵢE_{iib}E₄ | | E₄* | E_{b} |
| 458 | E_{1b}EᵢᵢE_{iib}E₄ | | E₄* | E_{b} |
| 459 | E₁ₐE₅EᵢE₄ | | E₄* | E_{b} |
| 460 | E_{1b}E₅EᵢE₄ | | E₄* | E_{b} |
| 461 | E₁ₐE₅EᵢᵢE₄ | | E₄* | E_{b} |
| 462 | E_{1b}E₅EᵢᵢE₄ | | E₄* | E_{b} |
| 463 | E₁ₐE₅EᵢᵢE_{iib}E₄ | | E₄* | E_{b} |
| 464 | E_{1b}E₅EᵢᵢE_{iib}E₄ | | E₄* | E_{b} |
| 465 | E₁ₐEᵢᵢᵢ | | E₄* | E_{b} |
| 466 | E_{1b}Eᵢᵢᵢ | | E₄* | E_{b} |
| 467 | E₁ₐE₅Eᵢᵢᵢ | | E₄* | E_{b} |
| 468 | E_{1b}E₅Eᵢᵢᵢ | | E₄* | E_{b} |
| 469 | E₁ₐEᵢᵢᵢE₄ | | E₄* | E_{b} |
| 470 | E_{1b}EᵢᵢᵢE₄ | | E₄* | E_{b} |
| 471 | E₁ₐE₅EᵢᵢᵢE₄ | | E₄* | E_{b} |
| 472 | E_{1b}E₅EᵢᵢᵢE₄ | | E₄* | E_{b} |
| 473 | E₁ₐEᵢᵥ | | E₄* | E_{b} |
| 474 | E_{1b}Eᵢᵥ | | E₄* | E_{b} |
| 475 | E₁ₐE₅Eᵢᵥ | | E₄* | E_{b} |
| 476 | E_{1b}E₅Eᵢᵥ | | E₄* | E_{b} |
| 477 | E₁ₐEᵢᵥE₄ | | E₄* | E_{b} |
| 478 | E_{1b}EᵢᵥE₄ | | E₄* | E_{b} |
| 479 | E₁ₐE₅EᵢᵥE₄ | | E₄* | E_{b} |
| 480 | E_{1b}E₅EᵢᵥE₄ | | E₄* | E_{b} |
| 481 | E₁ₐ | AlkL | | E_{b} |
| 482 | E_{1b} | AlkL | | E_{b} |
| 483 | E₁ₐE₅ | AlkL | | E_{b} |
| 484 | E_{1b}E₅ | AlkL | | E_{b} |
| 485 | E₁ₐEᵢ | AlkL | | E_{b} |
| 486 | E_{1b}Eᵢ | AlkL | | E_{b} |
| 487 | E₁ₐEᵢᵢ | AlkL | | E_{b} |
| 488 | E_{1b}Eᵢᵢ | AlkL | | E_{b} |
| 489 | E₁ₐEᵢᵢE_{iib} | AlkL | | E_{b} |
| 490 | E_{1b}EᵢᵢE_{iib} | AlkL | | E_{b} |
| 491 | E₁ₐE₅Eᵢ | AlkL | | E_{b} |
| 492 | E_{1b}E₅Eᵢ | AlkL | | E_{b} |
| 493 | E₁ₐE₅Eᵢᵢ | AlkL | | E_{b} |
| 494 | E_{1b}E₅Eᵢᵢ | AlkL | | E_{b} |
| 495 | E₁ₐE₅EᵢᵢE_{iib} | AlkL | | E_{b} |
| 496 | E_{1b}E₅EᵢᵢE_{iib} | AlkL | | E_{b} |
| 497 | E₁ₐE₄ | AlkL | | E_{b} |
| 498 | E_{1b}E₄ | AlkL | | E_{b} |
| 499 | E₁ₐE₅E₄ | AlkL | | E_{b} |
| 500 | E_{1b}E₅E₄ | AlkL | | E_{b} |
| 501 | E₁ₐEᵢE₄ | AlkL | | E_{b} |
| 502 | E_{1b}EᵢE₄ | AlkL | | E_{b} |
| 503 | E₁ₐEᵢᵢE₄ | AlkL | | E_{b} |
| 504 | E_{1b}EᵢᵢE₄ | AlkL | | E_{b} |
| 505 | E₁ₐEᵢᵢE_{iib}E₄ | AlkL | | E_{b} |
| 506 | E_{1b}EᵢᵢE_{iib}E₄ | AlkL | | E_{b} |
| 507 | E₁ₐE₅EᵢE₄ | AlkL | | E_{b} |
| 508 | E_{1b}E₅EᵢE₄ | AlkL | | E_{b} |
| 509 | E₁ₐE₅EᵢᵢE₄ | AlkL | | E_{b} |
| 510 | E_{1b}E₅EᵢᵢE₄ | AlkL | | E_{b} |
| 511 | E₁ₐE₅EᵢᵢE_{iib}E₄ | AlkL | | E_{b} |
| 512 | E_{1b}E₅EᵢᵢE_{iib}E₄ | AlkL | | E_{b} |
| 513 | E₁ₐEᵢᵢᵢ | AlkL | | E_{b} |
| 514 | E_{1b}Eᵢᵢᵢ | AlkL | | E_{b} |
| 515 | E₁ₐE₅Eᵢᵢᵢ | AlkL | | E_{b} |
| 516 | E_{1b}E₅Eᵢᵢᵢ | AlkL | | E_{b} |
| 517 | E₁ₐEᵢᵢᵢE₄ | AlkL | | E_{b} |
| 518 | E_{1b}EᵢᵢᵢE₄ | AlkL | | E_{b} |
| 519 | E₁ₐE₅EᵢᵢᵢE₄ | AlkL | | E_{b} |
| 520 | E_{1b}E₅EᵢᵢᵢE₄ | AlkL | | E_{b} |
| 521 | E₁ₐEᵢᵥ | AlkL | | E_{b} |
| 522 | E_{1b}Eᵢᵥ | AlkL | | E_{b} |
| 523 | E₁ₐE₅Eᵢᵥ | AlkL | | E_{b} |
| 524 | E_{1b}E₅Eᵢᵥ | AlkL | | E_{b} |
| 525 | E₁ₐEᵢᵥE₄ | AlkL | | E_{b} |
| 526 | E_{1b}EᵢᵥE₄ | AlkL | | E_{b} |
| 527 | E₁ₐE₅EᵢᵥE₄ | AlkL | | E_{b} |
| 528 | E_{1b}E₅EᵢᵥE₄ | AlkL | | E_{b} |
| 529 | E₁ₐ | AlkL | E₄* | E_{b} |
| 530 | E_{1b} | AlkL | E₄* | E_{b} |
| 531 | E₁ₐE₅ | AlkL | E₄* | E_{b} |
| 532 | E_{1b}E₅ | AlkL | E₄* | E_{b} |
| 533 | E₁ₐEᵢ | AlkL | E₄* | E_{b} |
| 534 | E_{1b}Eᵢ | AlkL | E₄* | E_{b} |
| 535 | E₁ₐEᵢᵢ | AlkL | E₄* | E_{b} |
| 536 | E_{1b}Eᵢᵢ | AlkL | E₄* | E_{b} |
| 537 | E₁ₐEᵢᵢE_{iib} | AlkL | E₄* | E_{b} |
| 538 | E_{1b}EᵢᵢE_{iib} | AlkL | E₄* | E_{b} |
| 539 | E₁ₐE₅Eᵢ | AlkL | E₄* | E_{b} |
| 540 | E_{1b}E₅Eᵢ | AlkL | E₄* | E_{b} |
| 541 | E₁ₐE₅Eᵢᵢ | AlkL | E₄* | E_{b} |
| 542 | E_{1b}E₅Eᵢᵢ | AlkL | E₄* | E_{b} |
| 543 | E₁ₐE₅EᵢᵢE_{iib} | AlkL | E₄* | E_{b} |
| 544 | E_{1b}E₅EᵢᵢE_{iib} | AlkL | E₄* | E_{b} |
| 545 | E₁ₐE₄ | AlkL | E₄* | E_{b} |
| 546 | E_{1b}E₄ | AlkL | E₄* | E_{b} |
| 547 | E₁ₐE₅E₄ | AlkL | E₄* | E_{b} |
| 548 | E_{1b}E₅E₄ | AlkL | E₄* | E_{b} |
| 549 | E₁ₐEᵢE₄ | AlkL | E₄* | E_{b} |
| 550 | E_{1b}EᵢE₄ | AlkL | E₄* | E_{b} |
| 551 | E₁ₐEᵢᵢE₄ | AlkL | E₄* | E_{b} |
| 552 | E_{1b}EᵢᵢE₄ | AlkL | E₄* | E_{b} |
| 553 | E₁ₐEᵢᵢE_{iib}E₄ | AlkL | E₄* | E_{b} |
| 554 | E_{1b}EᵢᵢE_{iib}E₄ | AlkL | E₄* | E_{b} |
| 555 | E₁ₐE₅EᵢE₄ | AlkL | E₄* | E_{b} |
| 556 | E_{1b}E₅EᵢE₄ | AlkL | E₄* | E_{b} |
| 557 | E₁ₐE₅EᵢᵢE₄ | AlkL | E₄* | E_{b} |
| 558 | E_{1b}E₅EᵢᵢE₄ | AlkL | E₄* | E_{b} |
| 559 | E₁ₐE₅EᵢᵢE_{iib}E₄ | AlkL | E₄* | E_{b} |
| 560 | E_{1b}E₅EᵢᵢE_{iib}E₄ | AlkL | E₄* | E_{b} |
| 561 | E₁ₐEᵢᵢᵢ | AlkL | E₄* | E_{b} |
| 562 | E_{1b}Eᵢᵢᵢ | AlkL | E₄* | E_{b} |
| 563 | E₁ₐE₅Eᵢᵢᵢ | AlkL | E₄* | E_{b} |
| 564 | E_{1b}E₅Eᵢᵢᵢ | AlkL | E₄* | E_{b} |
| 565 | E₁ₐEᵢᵢᵢE₄ | AlkL | E₄* | E_{b} |
| 566 | E_{1b}EᵢᵢᵢE₄ | AlkL | E₄* | E_{b} |
| 567 | E₁ₐE₅EᵢᵢᵢE₄ | AlkL | E₄* | E_{b} |
| 568 | E_{1b}E₅EᵢᵢᵢE₄ | AlkL | E₄* | E_{b} |
| 569 | E₁ₐEᵢᵥ | AlkL | E₄* | E_{b} |
| 570 | E_{1b}Eᵢᵥ | AlkL | E₄* | E_{b} |
| 571 | E₁ₐE₅Eᵢᵥ | AlkL | E₄* | E_{b} |
| 572 | E_{1b}E₅Eᵢᵥ | AlkL | E₄* | E_{b} |
| 573 | E₁ₐEᵢᵥE₄ | AlkL | E₄* | E_{b} |
| 574 | E_{1b}EᵢᵥE₄ | AlkL | E₄* | E_{b} |
| 575 | E₁ₐE₅EᵢᵥE₄ | AlkL | E₄* | E_{b} |
| 576 | E_{1b}E₅EᵢᵥE₄ | AlkL | E₄* | E_{b} |
| 577 | E₁ₐ | | | E_{d} |
| 578 | E_{1b} | | | E_{d} |
| 579 | E₁ₐE₅ | | | E_{d} |
| 580 | E_{1b}E₅ | | | E_{d} |
| 581 | E₁ₐEᵢ | | | E_{d} |
| 582 | E_{1b}Eᵢ | | | E_{d} |
| 583 | E₁ₐEᵢᵢ | | | E_{d} |
| 584 | E_{1b}Eᵢᵢ | | | E_{d} |
| 585 | E₁ₐEᵢᵢE_{iib} | | | E_{d} |
| 586 | E_{1b}EᵢᵢE_{iib} | | | E_{d} |
| 587 | E₁ₐE₅Eᵢ | | | E_{d} |
| 588 | E_{1b}E₅Eᵢ | | | E_{d} |
| 589 | E₁ₐE₅Eᵢᵢ | | | E_{d} |
| 590 | E_{1b}E₅Eᵢᵢ | | | E_{d} |
| 591 | E₁ₐE₅EᵢᵢE_{iib} | | | E_{d} |
| 592 | E_{1b}E₅EᵢᵢE_{iib} | | | E_{d} |
| 593 | E₁ₐE₄ | | | E_{d} |
| 594 | E_{1b}E₄ | | | E_{d} |
| 595 | E₁ₐE₅E₄ | | | E_{d} |
| 596 | E_{1b}E₅E₄ | | | E_{d} |
| 597 | E₁ₐEᵢE₄ | | | E_{d} |
| 598 | E_{1b}EᵢE₄ | | | E_{d} |
| 599 | E₁ₐEᵢᵢE₄ | | | E_{d} |
| 600 | E_{1b}EᵢᵢE₄ | | | E_{d} |
| 601 | E₁ₐEᵢᵢE_{iib}E₄ | | | E_{d} |
| 602 | E_{1b}EᵢᵢE_{iib}E₄ | | | E_{d} |
| 603 | E₁ₐE₅EᵢE₄ | | | E_{d} |
| 604 | E_{1b}E₅EᵢE₄ | | | E_{d} |
| 605 | E₁ₐE₅EᵢᵢE₄ | | | E_{d} |
| 606 | E_{1b}E₅EᵢᵢE₄ | | | E_{d} |
| 607 | E₁ₐE₅EᵢᵢE_{iib}E₄ | | | E_{d} |
| 608 | E_{1b}E₅EᵢᵢE_{iib}E₄ | | | E_{d} |
| 609 | E₁ₐEᵢᵢᵢ | | | E_{d} |
| 610 | E_{1b}Eᵢᵢᵢ | | | E_{d} |
| 611 | E₁ₐE₅Eᵢᵢᵢ | | | E_{d} |
| 612 | E_{1b}E₅Eᵢᵢᵢ | | | E_{d} |
| 613 | E₁ₐEᵢᵢᵢE₄ | | | E_{d} |
| 614 | E_{1b}EᵢᵢᵢE₄ | | | E_{d} |
| 615 | E₁ₐE₅EᵢᵢᵢE₄ | | | E_{d} |
| 616 | E_{1b}E₅EᵢᵢᵢE₄ | | | E_{d} |
| 617 | E₁ₐEᵢᵥ | | | E_{d} |
| 618 | E_{1b}Eᵢᵥ | | | E_{d} |
| 619 | E₁ₐE₅Eᵢᵥ | | | E_{d} |
| 620 | E_{1b}E₅Eᵢᵥ | | | E_{d} |
| 621 | E₁ₐEᵢᵥE₄ | | | E_{d} |
| 622 | E_{1b}EᵢᵥE₄ | | | E_{d} |
| 623 | E₁ₐE₅EᵢᵥE₄ | | | E_{d} |
| 624 | E_{1b}E₅EᵢᵥE₄ | | | E_{d} |
| 625 | E₁ₐ | | E₄* | E_{d} |
| 626 | E_{1b} | | E₄* | E_{d} |
| 627 | E₁ₐE₅ | | E₄* | E_{d} |
| 628 | E_{1b}E₅ | | E₄* | E_{d} |
| 629 | E₁ₐEᵢ | | E₄* | E_{d} |
| 630 | E_{1b}Eᵢ | | E₄* | E_{d} |
| 631 | E₁ₐEᵢᵢ | | E₄* | E_{d} |
| 632 | E_{1b}Eᵢᵢ | | E₄* | E_{d} |
| 633 | E₁ₐEᵢᵢE_{iib} | | E₄* | E_{d} |
| 634 | E_{1b}EᵢᵢE_{iib} | | E₄* | E_{d} |
| 635 | E₁ₐE₅Eᵢ | | E₄* | E_{d} |
| 636 | E_{1b}E₅Eᵢ | | E₄* | E_{d} |
| 637 | E₁ₐE₅Eᵢᵢ | | E₄* | E_{d} |
| 638 | E_{1b}E₅Eᵢᵢ | | E₄* | E_{d} |
| 639 | E₁ₐE₅EᵢᵢE_{iib} | | E₄* | E_{d} |
| 640 | E_{1b}E₅EᵢᵢE_{iib} | | E₄* | E_{d} |
| 641 | E₁ₐE₄ | | E₄* | E_{d} |
| 642 | E_{1b}E₄ | | E₄* | E_{d} |
| 643 | E₁ₐE₅E₄ | | E₄* | E_{d} |
| 644 | E_{1b}E₅E₄ | | E₄* | E_{d} |
| 645 | E₁ₐEᵢE₄ | | E₄* | E_{d} |
| 646 | E_{1b}EᵢE₄ | | E₄* | E_{d} |
| 647 | E₁ₐEᵢᵢE₄ | | E₄* | E_{d} |
| 648 | E_{1b}EᵢᵢE₄ | | E₄* | E_{d} |
| 649 | E₁ₐEᵢᵢE_{iib}E₄ | | E₄* | E_{d} |
| 650 | E_{1b}EᵢᵢE_{iib}E₄ | | E₄* | E_{d} |
| 651 | E₁ₐE₅EᵢE₄ | | E₄* | E_{d} |
| 652 | E_{1b}E₅EᵢE₄ | | E₄* | E_{d} |
| 653 | E₁ₐE₅EᵢᵢE₄ | | E₄* | E_{d} |
| 654 | E_{1b}E₅EᵢᵢE₄ | | E₄* | E_{d} |
| 655 | E₁ₐE₅EᵢᵢE_{iib}E₄ | | E₄* | E_{d} |
| 656 | E_{1b}E₅EᵢᵢE_{iib}E₄ | | E₄* | E_{d} |
| 657 | E₁ₐEᵢᵢᵢ | | E₄* | E_{d} |
| 658 | E_{1b}Eᵢᵢᵢ | | E₄* | E_{d} |
| 659 | E₁ₐE₅Eᵢᵢᵢ | | E₄* | E_{d} |
| 660 | E_{1b}E₅Eᵢᵢᵢ | | E₄* | E_{d} |
| 661 | E₁ₐEᵢᵢᵢE₄ | | E₄* | E_{d} |
| 662 | E_{1b}EᵢᵢᵢE₄ | | E₄* | E_{d} |
| 663 | E₁ₐE₅EᵢᵢᵢE₄ | | E₄* | E_{d} |
| 664 | E_{1b}E₅EᵢᵢᵢE₄ | | E₄* | E_{d} |
| 665 | E₁ₐEᵢᵥ | | E₄* | E_{d} |
| 666 | E_{1b}Eᵢᵥ | | E₄* | E_{d} |
| 667 | E₁ₐE₅Eᵢᵥ | | E₄* | E_{d} |
| 668 | E_{1b}E₅Eᵢᵥ | | E₄* | E_{d} |
| 669 | E₁ₐEᵢᵥE₄ | | E₄* | E_{d} |
| 670 | E_{1b}EᵢᵥE₄ | | E₄* | E_{d} |
| 671 | E₁ₐE₅EᵢᵥE₄ | | E₄* | E_{d} |
| 672 | E_{1b}E₅EᵢᵥE₄ | | E₄* | E_{d} |
| 673 | E₁ₐ | AlkL | | E_{d} |
| 674 | E_{1b} | AlkL | | E_{d} |
| 675 | E₁ₐE₅ | AlkL | | E_{d} |
| 676 | E_{1b}E₅ | AlkL | | E_{d} |
| 677 | E₁ₐEᵢ | AlkL | | E_{d} |
| 678 | E_{1b}Eᵢ | AlkL | | E_{d} |
| 679 | E₁ₐEᵢᵢ | AlkL | | E_{d} |
| 680 | E_{1b}Eᵢᵢ | AlkL | | E_{d} |
| 681 | E₁ₐEᵢᵢE_{iib} | AlkL | | E_{d} |
| 682 | E_{1b}EᵢᵢE_{iib} | AlkL | | E_{d} |
| 683 | E₁ₐE₅Eᵢ | AlkL | | E_{d} |
| 684 | E_{1b}E₅Eᵢ | AlkL | | E_{d} |
| 685 | E₁ₐE₅Eᵢᵢ | AlkL | | E_{d} |
| 686 | E_{1b}E₅Eᵢᵢ | AlkL | | E_{d} |
| 687 | E₁ₐE₅EᵢᵢE_{iib} | AlkL | | E_{d} |
| 688 | E_{1b}E₅EᵢᵢE_{iib} | AlkL | | E_{d} |
| 689 | E₁ₐE₄ | AlkL | | E_{d} |
| 690 | E_{1b}E₄ | AlkL | | E_{d} |
| 691 | E₁ₐE₅E₄ | AlkL | | E_{d} |
| 692 | E_{1b}E₅E₄ | AlkL | | E_{d} |
| 693 | E₁ₐEᵢE₄ | AlkL | | E_{d} |
| 694 | E_{1b}EᵢE₄ | AlkL | | E_{d} |
| 695 | E₁ₐEᵢᵢE₄ | AlkL | | E_{d} |
| 696 | E_{1b}EᵢᵢE₄ | AlkL | | E_{d} |
| 697 | E₁ₐEᵢᵢE_{iib}E₄ | AlkL | | E_{d} |
| 698 | E_{1b}EᵢᵢE_{iib}E₄ | AlkL | | E_{d} |
| 699 | E₁ₐE₅EᵢE₄ | AlkL | | E_{d} |
| 700 | E_{1b}E₅EᵢE₄ | AlkL | | E_{d} |
| 701 | E₁ₐE₅EᵢᵢE₄ | AlkL | | E_{d} |
| 702 | E_{1b}E₅EᵢᵢE₄ | AlkL | | E_{d} |
| 703 | E₁ₐE₅EᵢᵢE_{iib}E₄ | AlkL | | E_{d} |
| 704 | E_{1b}E₅EᵢᵢE_{iib}E₄ | AlkL | | E_{d} |
| 705 | E₁ₐEᵢᵢᵢ | AlkL | | E_{d} |
| 706 | E_{1b}Eᵢᵢᵢ | AlkL | | E_{d} |
| 707 | E₁ₐE₅Eᵢᵢᵢ | AlkL | | E_{d} |
| 708 | E_{1b}E₅Eᵢᵢᵢ | AlkL | | E_{d} |
| 709 | E₁ₐEᵢᵢᵢE₄ | AlkL | | E_{d} |
| 710 | E_{1b}EᵢᵢᵢE₄ | AlkL | | E_{d} |
| 711 | E₁ₐE₅EᵢᵢᵢE₄ | AlkL | | E_{d} |
| 712 | E_{1b}E₅EᵢᵢᵢE₄ | AlkL | | E_{d} |
| 713 | E₁ₐEᵢᵥ | AlkL | | E_{d} |
| 714 | E_{1b}Eᵢᵥ | AlkL | | E_{d} |
| 715 | E₁ₐE₅Eᵢᵥ | AlkL | | E_{d} |
| 716 | E_{1b}E₅Eᵢᵥ | AlkL | | E_{d} |
| 717 | E₁ₐEᵢᵥE₄ | AlkL | | E_{d} |
| 718 | E_{1b}EᵢᵥE₄ | AlkL | | E_{d} |
| 719 | E₁ₐE₅EᵢᵥE₄ | AlkL | | E_{d} |
| 720 | E_{1b}E₅EᵢᵥE₄ | AlkL | | E_{d} |
| 721 | E₁ₐ | AlkL | E₄* | E_{d} |
| 722 | E_{1b} | AlkL | E₄* | E_{d} |
| 723 | E₁ₐE₅ | AlkL | E₄* | E_{d} |
| 724 | E_{1b}E₅ | AlkL | E₄* | E_{d} |
| 725 | E₁ₐEᵢ | AlkL | E₄* | E_{d} |
| 726 | E_{1b}Eᵢ | AlkL | E₄* | E_{d} |
| 727 | E₁ₐEᵢᵢ | AlkL | E₄* | E_{d} |
| 728 | E_{1b}Eᵢᵢ | AlkL | E₄* | E_{d} |
| 729 | E₁ₐEᵢᵢE_{iib} | AlkL | E₄* | E_{d} |
| 730 | E_{1b}EᵢᵢE_{iib} | AlkL | E₄* | E_{d} |
| 731 | E₁ₐE₅Eᵢ | AlkL | E₄* | E_{d} |
| 732 | E_{1b}E₅Eᵢ | AlkL | E₄* | E_{d} |
| 733 | E₁ₐE₅Eᵢᵢ | AlkL | E₄* | E_{d} |
| 734 | E_{1b}E₅Eᵢᵢ | AlkL | E₄* | E_{d} |
| 735 | E₁ₐE₅EᵢᵢE_{iib} | AlkL | E₄* | E_{d} |
| 736 | E_{1b}E₅EᵢᵢE_{iib} | AlkL | E₄* | E_{d} |
| 737 | E₁ₐE₄ | AlkL | E₄* | E_{d} |
| 738 | E_{1b}E₄ | AlkL | E₄* | E_{d} |
| 739 | E₁ₐE₅E₄ | AlkL | E₄* | E_{d} |
| 740 | E_{1b}E₅E₄ | AlkL | E₄* | E_{d} |
| 741 | E₁ₐEᵢE₄ | AlkL | E₄* | E_{d} |
| 742 | E_{1b}EᵢE₄ | AlkL | E₄* | E_{d} |
| 743 | E₁ₐEᵢᵢE₄ | AlkL | E₄* | E_{d} |
| 744 | E_{1b}EᵢᵢE₄ | AlkL | E₄* | E_{d} |
| 745 | E₁ₐEᵢᵢE_{iib}E₄ | AlkL | E₄* | E_{d} |
| 746 | E_{1b}EᵢᵢE_{iib}E₄ | AlkL | E₄* | E_{d} |
| 747 | E₁ₐE₅EᵢE₄ | AlkL | E₄* | E_{d} |
| 748 | E_{1b}E₅EᵢE₄ | AlkL | E₄* | E_{d} |
| 749 | E₁ₐE₅EᵢᵢE₄ | AlkL | E₄* | E_{d} |
| 750 | E_{1b}E₅EᵢᵢE₄ | AlkL | E₄* | E_{d} |
| 751 | E₁ₐE₅EᵢᵢE_{iib}E₄ | AlkL | E₄* | E_{d} |
| 752 | E_{1b}E₅EᵢᵢE_{iib}E₄ | AlkL | E₄* | E_{d} |
| 753 | E₁ₐEᵢᵢᵢ | AlkL | E₄* | E_{d} |
| 754 | E_{1b}Eᵢᵢᵢ | AlkL | E₄* | E_{d} |
| 755 | E₁ₐE₅Eᵢᵢᵢ | AlkL | E₄* | E_{d} |
| 756 | E_{1b}E₅Eᵢᵢᵢ | AlkL | E₄* | E_{d} |
| 757 | E₁ₐEᵢᵢᵢE₄ | AlkL | E₄* | E_{d} |
| 758 | E_{1b}EᵢᵢᵢE₄ | AlkL | E₄* | E_{d} |
| 759 | E₁ₐE₅EᵢᵢᵢE₄ | AlkL | E₄* | E_{d} |
| 760 | E_{1b}E₅EᵢᵢᵢE₄ | AlkL | E₄* | E_{d} |
| 761 | E₁ₐEᵢᵥ | AlkL | E₄* | E_{d} |
| 762 | E_{1b}Eᵢᵥ | AlkL | E₄* | E_{d} |
| 763 | E₁ₐE₅Eᵢᵥ | AlkL | E₄* | E_{d} |
| 764 | E_{1b}E₅Eᵢᵥ | AlkL | E₄* | E_{d} |
| 765 | E₁ₐEᵢᵥE₄ | AlkL | E₄* | E_{d} |
| 766 | E_{1b}EᵢᵥE₄ | AlkL | E₄* | E_{d} |
| 767 | E₁ₐE₅EᵢᵥE₄ | AlkL | E₄* | E_{d} |
| 768 | E_{1b}E₅EᵢᵥE₄ | AlkL | E₄* | E_{d} |
| 769 | E₁ₐ | | | Eₑ |
| 770 | E_{1b} | | | Eₑ |
| 771 | E₁ₐE₅ | | | Eₑ |
| 772 | E_{1b}E₅ | | | Eₑ |
| 773 | E₁ₐEᵢ | | | Eₑ |
| 774 | E_{1b}Eᵢ | | | Eₑ |
| 775 | E₁ₐEᵢᵢ | | | Eₑ |
| 776 | E_{1b}Eᵢᵢ | | | Eₑ |
| 777 | E₁ₐEᵢᵢE_{iib} | | | Eₑ |
| 778 | E_{1b}EᵢᵢE_{iib} | | | Eₑ |
| 779 | E₁ₐE₅Eᵢ | | | Eₑ |
| 780 | E_{1b}E₅Eᵢ | | | Eₑ |
| 781 | E₁ₐE₅Eᵢᵢ | | | Eₑ |
| 782 | E_{1b}E₅Eᵢᵢ | | | Eₑ |
| 783 | E₁ₐE₅EᵢᵢE_{iib} | | | Eₑ |
| 784 | E_{1b}E₅EᵢᵢE_{iib} | | | Eₑ |
| 785 | E₁ₐE₄ | | | Eₑ |
| 786 | E_{1b}E₄ | | | Eₑ |
| 787 | E₁ₐE₅E₄ | | | Eₑ |
| 788 | E_{1b}E₅E₄ | | | Eₑ |
| 789 | E₁ₐEᵢE₄ | | | Eₑ |
| 790 | E_{1b}EᵢE₄ | | | Eₑ |
| 791 | E₁ₐEᵢᵢE₄ | | | Eₑ |
| 792 | E_{1b}EᵢᵢE₄ | | | Eₑ |
| 793 | E₁ₐEᵢᵢE_{iib}E₄ | | | Eₑ |
| 794 | E_{1b}EᵢᵢE_{iib}E₄ | | | Eₑ |
| 795 | E₁ₐE₅EᵢE₄ | | | Eₑ |
| 796 | E_{1b}E₅EᵢE₄ | | | Eₑ |
| 797 | E₁ₐE₅EᵢᵢE₄ | | | Eₑ |
| 798 | E_{1b}E₅EᵢᵢE₄ | | | Eₑ |
| 799 | E₁ₐE₅EᵢᵢE_{iib}E₄ | | | Eₑ |
| 800 | E_{1b}E₅EᵢᵢE_{iib}E₄ | | | Eₑ |
| 801 | E₁ₐEᵢᵢᵢ | | | Eₑ |
| 802 | E_{1b}Eᵢᵢᵢ | | | Eₑ |
| 803 | E₁ₐE₅Eᵢᵢᵢ | | | Eₑ |
| 804 | E_{1b}E₅Eᵢᵢᵢ | | | Eₑ |
| 805 | E₁ₐEᵢᵢᵢE₄ | | | Eₑ |
| 806 | E_{1b}EᵢᵢᵢE₄ | | | Eₑ |
| 807 | E₁ₐE₅EᵢᵢᵢE₄ | | | Eₑ |
| 808 | E_{1b}E₅EᵢᵢᵢE₄ | | | Eₑ |
| 809 | E₁ₐEᵢᵥ | | | Eₑ |
| 810 | E_{1b}Eᵢᵥ | | | Eₑ |
| 811 | E₁ₐE₅Eᵢᵥ | | | Eₑ |
| 812 | E_{1b}E₅Eᵢᵥ | | | Eₑ |
| 813 | E₁ₐEᵢᵥE₄ | | | Eₑ |
| 814 | E_{1b}EᵢᵥE₄ | | | Eₑ |
| 815 | E₁ₐE₅EᵢᵥE₄ | | | Eₑ |
| 816 | E_{1b}E₅EᵢᵥE₄ | | | Eₑ |
| 817 | E₁ₐ | | E₄* | Eₑ |
| 818 | E_{1b} | | E₄* | Eₑ |
| 819 | E₁ₐE₅ | | E₄* | Eₑ |
| 820 | E_{1b}E₅ | | E₄* | Eₑ |
| 821 | E₁ₐEᵢ | | E₄* | Eₑ |
| 822 | E_{1b}Eᵢ | | E₄* | Eₑ |
| 823 | E₁ₐEᵢᵢ | | E₄* | Eₑ |
| 824 | E_{1b}Eᵢᵢ | | E₄* | Eₑ |
| 825 | E₁ₐEᵢᵢE_{iib} | | E₄* | Eₑ |
| 826 | E_{1b}EᵢᵢE_{iib} | | E₄* | Eₑ |
| 827 | E₁ₐE₅Eᵢ | | E₄* | Eₑ |
| 828 | E_{1b}E₅Eᵢ | | E₄* | Eₑ |
| 829 | E₁ₐE₅Eᵢᵢ | | E₄* | Eₑ |
| 830 | E_{1b}E₅Eᵢᵢ | | E₄* | Eₑ |
| 831 | E₁ₐE₅EᵢᵢE_{iib} | | E₄* | Eₑ |
| 832 | E_{1b}E₅EᵢᵢE_{iib} | | E₄* | Eₑ |
| 833 | E₁ₐE₄ | | E₄* | Eₑ |
| 834 | E_{1b}E₄ | | E₄* | Eₑ |
| 835 | E₁ₐE₅E₄ | | E₄* | Eₑ |
| 836 | E_{1b}E₅E₄ | | E₄* | Eₑ |
| 837 | E₁ₐEᵢE₄ | | E₄* | Eₑ |
| 838 | E_{1b}EᵢE₄ | | E₄* | Eₑ |
| 839 | E₁ₐEᵢᵢE₄ | | E₄* | Eₑ |
| 840 | E_{1b}EᵢᵢE₄ | | E₄* | Eₑ |
| 841 | E₁ₐEᵢᵢE_{iib}E₄ | | E₄* | Eₑ |
| 842 | E_{1b}EᵢᵢE_{iib}E₄ | | E₄* | Eₑ |
| 843 | E₁ₐE₅EᵢE₄ | | E₄* | Eₑ |
| 844 | E_{1b}E₅EᵢE₄ | | E₄* | Eₑ |
| 845 | E₁ₐE₅EᵢᵢE₄ | | E₄* | Eₑ |
| 846 | E_{1b}E₅EᵢᵢE₄ | | E₄* | Eₑ |
| 847 | E₁ₐE₅EᵢᵢE_{iib}E₄ | | E₄* | Eₑ |
| 848 | E_{1b}E₅EᵢᵢE_{iib}E₄ | | E₄* | Eₑ |
| 849 | E₁ₐEᵢᵢᵢ | | E₄* | Eₑ |
| 850 | E_{1b}Eᵢᵢᵢ | | E₄* | Eₑ |
| 851 | E₁ₐE₅Eᵢᵢᵢ | | E₄* | Eₑ |
| 852 | E_{1b}E₅Eᵢᵢᵢ | | E₄* | Eₑ |
| 853 | E₁ₐEᵢᵢᵢE₄ | | E₄* | Eₑ |
| 854 | E_{1b}EᵢᵢᵢE₄ | | E₄* | Eₑ |
| 855 | E₁ₐE₅EᵢᵢᵢE₄ | | E₄* | Eₑ |
| 856 | E_{1b}E₅EᵢᵢᵢE₄ | | E₄* | Eₑ |
| 857 | E₁ₐEᵢᵥ | | E₄* | Eₑ |
| 858 | E_{1b}Eᵢᵥ | | E₄* | Eₑ |
| 859 | E₁ₐE₅Eᵢᵥ | | E₄* | Eₑ |
| 860 | E_{1b}E₅Eᵢᵥ | | E₄* | Eₑ |
| 861 | E₁ₐEᵢᵥE₄ | | E₄* | Eₑ |
| 862 | E_{1b}EᵢᵥE₄ | | E₄* | Eₑ |
| 863 | E₁ₐE₅EᵢᵥE₄ | | E₄* | Eₑ |
| 864 | E_{1b}E₅EᵢᵥE₄ | | E₄* | Eₑ |
| 865 | E₁ₐ | AlkL | | Eₑ |
| 866 | E_{1b} | AlkL | | Eₑ |
| 867 | E₁ₐE₅ | AlkL | | Eₑ |
| 868 | E_{1b}E₅ | AlkL | | Eₑ |
| 869 | E₁ₐEᵢ | AlkL | | Eₑ |
| 870 | E_{1b}Eᵢ | AlkL | | Eₑ |
| 871 | E₁ₐEᵢᵢ | AlkL | | Eₑ |
| 872 | E_{1b}Eᵢᵢ | AlkL | | Eₑ |
| 873 | E₁ₐEᵢᵢE_{iib} | AlkL | | Eₑ |
| 874 | E_{1b}EᵢᵢE_{iib} | AlkL | | Eₑ |
| 875 | E₁ₐE₅Eᵢ | AlkL | | Eₑ |
| 876 | E_{1b}E₅Eᵢ | AlkL | | Eₑ |
| 877 | E₁ₐE₅Eᵢᵢ | AlkL | | Eₑ |
| 878 | E_{1b}E₅Eᵢᵢ | AlkL | | Eₑ |
| 879 | E₁ₐE₅EᵢᵢE_{iib} | AlkL | | Eₑ |
| 880 | E_{1b}E₅EᵢᵢE_{iib} | AlkL | | Eₑ |
| 881 | E₁ₐE₄ | AlkL | | Eₑ |
| 882 | E_{1b}E₄ | AlkL | | Eₑ |
| 883 | E₁ₐE₅E₄ | AlkL | | Eₑ |
| 884 | E_{1b}E₅E₄ | AlkL | | Eₑ |
| 885 | E₁ₐEᵢE₄ | AlkL | | Eₑ |
| 886 | E_{1b}EᵢE₄ | AlkL | | Eₑ |
| 887 | E₁ₐEᵢᵢE₄ | AlkL | | Eₑ |
| 888 | E_{1b}EᵢᵢE₄ | AlkL | | Eₑ |
| 889 | E₁ₐEᵢᵢE_{iib}E₄ | AlkL | | Eₑ |
| 890 | E_{1b}EᵢᵢE_{iib}E₄ | AlkL | | Eₑ |
| 891 | E₁ₐE₅EᵢE₄ | AlkL | | Eₑ |
| 892 | E_{1b}E₅EᵢE₄ | AlkL | | Eₑ |
| 893 | E₁ₐE₅EᵢᵢE₄ | AlkL | | Eₑ |
| 894 | E_{1b}E₅EᵢᵢE₄ | AlkL | | Eₑ |
| 895 | E₁ₐE₅EᵢᵢE_{iib}E₄ | AlkL | | Eₑ |
| 896 | E_{1b}E₅EᵢᵢE_{iib}E₄ | AlkL | | Eₑ |
| 897 | E₁ₐEᵢᵢᵢ | AlkL | | Eₑ |
| 898 | E_{1b}Eᵢᵢᵢ | AlkL | | Eₑ |
| 899 | E₁ₐE₅Eᵢᵢᵢ | AlkL | | Eₑ |
| 900 | E_{1b}E₅Eᵢᵢᵢ | AlkL | | Eₑ |
| 901 | E₁ₐEᵢᵢᵢE₄ | AlkL | | Eₑ |
| 902 | E_{1b}EᵢᵢᵢE₄ | AlkL | | Eₑ |
| 903 | E₁ₐE₅EᵢᵢᵢE₄ | AlkL | | Eₑ |
| 904 | E_{1b}E₅EᵢᵢᵢE₄ | AlkL | | Eₑ |
| 905 | E₁ₐEᵢᵥ | AlkL | | Eₑ |
| 906 | E_{1b}Eᵢᵥ | AlkL | | Eₑ |
| 907 | E₁ₐE₅Eᵢᵥ | AlkL | | Eₑ |
| 908 | E_{1b}E₅Eᵢᵥ | AlkL | | Eₑ |
| 909 | E₁ₐEᵢᵥE₄ | AlkL | | Eₑ |
| 910 | E_{1b}EᵢᵥE₄ | AlkL | | Eₑ |
| 911 | E₁ₐE₅EᵢᵥE₄ | AlkL | | Eₑ |
| 912 | E_{1b}E₅EᵢᵥE₄ | AlkL | | Eₑ |
| 913 | E₁ₐ | AlkL | E₄* | Eₑ |
| 914 | E_{1b} | AlkL | E₄* | Eₑ |
| 915 | E₁ₐE₅ | AlkL | E₄* | Eₑ |
| 916 | E_{1b}E₅ | AlkL | E₄* | Eₑ |
| 917 | E₁ₐEᵢ | AlkL | E₄* | Eₑ |
| 918 | E_{1b}Eᵢ | AlkL | E₄* | Eₑ |
| 919 | E₁ₐEᵢᵢ | AlkL | E₄* | Eₑ |
| 920 | E_{1b}Eᵢᵢ | AlkL | E₄* | Eₑ |
| 921 | E₁ₐEᵢᵢE_{iib} | AlkL | E₄* | Eₑ |
| 922 | E_{1b}EᵢᵢE_{iib} | AlkL | E₄* | Eₑ |
| 923 | E₁ₐE₅Eᵢ | AlkL | E₄* | Eₑ |
| 924 | E_{1b}E₅Eᵢ | AlkL | E₄* | Eₑ |
| 925 | E₁ₐE₅Eᵢᵢ | AlkL | E₄* | Eₑ |
| 926 | E_{1b}E₅Eᵢᵢ | AlkL | E₄* | Eₑ |
| 927 | E₁ₐE₅EᵢᵢE_{iib} | AlkL | E₄* | Eₑ |
| 928 | E_{1b}E₅EᵢᵢE_{iib} | AlkL | E₄* | Eₑ |
| 929 | E₁ₐE₄ | AlkL | E₄* | Eₑ |
| 930 | E_{1b}E₄ | AlkL | E₄* | Eₑ |
| 931 | E₁ₐE₅E₄ | AlkL | E₄* | Eₑ |
| 932 | E_{1b}E₅E₄ | AlkL | E₄* | Eₑ |
| 933 | E₁ₐEᵢE₄ | AlkL | E₄* | Eₑ |
| 934 | E_{1b}EᵢE₄ | AlkL | E₄* | Eₑ |
| 935 | E₁ₐEᵢᵢE₄ | AlkL | E₄* | Eₑ |
| 936 | E_{1b}EᵢᵢE₄ | AlkL | E₄* | Eₑ |
| 937 | E₁ₐEᵢᵢE_{iib}E₄ | AlkL | E₄* | Eₑ |
| 938 | E_{1b}EᵢᵢE_{iib}E₄ | AlkL | E₄* | Eₑ |
| 939 | E₁ₐE₅EᵢE₄ | AlkL | E₄* | Eₑ |
| 940 | E_{1b}E₅EᵢE₄ | AlkL | E₄* | Eₑ |
| 941 | E₁ₐE₅EᵢᵢE₄ | AlkL | E₄* | Eₑ |
| 942 | E_{1b}E₅EᵢᵢE₄ | AlkL | E₄* | Eₑ |
| 943 | E₁ₐE₅EᵢᵢE_{iib}E₄ | AlkL | E₄* | Eₑ |
| 944 | E_{1b}E₅EᵢᵢE_{iib}E₄ | AlkL | E₄* | Eₑ |
| 945 | E₁ₐEᵢᵢᵢ | AlkL | E₄* | Eₑ |
| 946 | E_{1b}Eᵢᵢᵢ | AlkL | E₄* | Eₑ |
| 947 | E₁ₐE₅Eᵢᵢᵢ | AlkL | E₄* | Eₑ |
| 948 | E_{1b}E₅Eᵢᵢᵢ | AlkL | E₄* | Eₑ |
| 949 | E₁ₐEᵢᵢᵢE₄ | AlkL | E₄* | Eₑ |
| 950 | E_{1b}EᵢᵢᵢE₄ | AlkL | E₄* | Eₑ |
| 951 | E₁ₐE₅EᵢᵢᵢE₄ | AlkL | E₄* | Eₑ |
| 952 | E_{1b}E₅EᵢᵢᵢE₄ | AlkL | E₄* | Eₑ |
| 953 | E₁ₐEᵢᵥ | AlkL | E₄* | Eₑ |
| 954 | E_{1b}Eᵢᵥ | AlkL | E₄* | Eₑ |
| 955 | E₁ₐE₅Eᵢᵥ | AlkL | E₄* | Eₑ |
| 956 | E_{1b}E₅Eᵢᵥ | AlkL | E₄* | Eₑ |
| 957 | E₁ₐEᵢᵥE₄ | AlkL | E₄* | Eₑ |
| 958 | E_{1b}EᵢᵥE₄ | AlkL | E₄* | Eₑ |
| 959 | E₁ₐE₅EᵢᵥE₄ | AlkL | E₄* | Eₑ |
| 960 | E_{1b}E₅EᵢᵥE₄ | AlkL | E₄* | Eₑ |
| 961 | E₁ₐ | | | E_{f} |
| 962 | E_{1b} | | | E_{f} |
| 963 | E₁ₐE₅ | | | E_{f} |
| 964 | E_{1b}E₅ | | | E_{f} |
| 965 | E₁ₐEᵢ | | | E_{f} |
| 966 | E_{1b}Eᵢ | | | E_{f} |
| 967 | E₁ₐEᵢᵢ | | | E_{f} |
| 968 | E_{1b}Eᵢᵢ | | | E_{f} |
| 969 | E₁ₐEᵢᵢE_{iib} | | | E_{f} |
| 970 | E_{1b}EᵢᵢE_{iib} | | | E_{f} |
| 971 | E₁ₐE₅Eᵢ | | | E_{f} |
| 972 | E_{1b}E₅Eᵢ | | | E_{f} |
| 973 | E₁ₐE₅Eᵢᵢ | | | E_{f} |
| 974 | E_{1b}E₅Eᵢᵢ | | | E_{f} |
| 975 | E₁ₐE₅EᵢᵢE_{iib} | | | E_{f} |
| 976 | E_{1b}E₅EᵢᵢE_{iib} | | | E_{f} |
| 977 | E₁ₐE₄ | | | E_{f} |
| 978 | E_{1b}E₄ | | | E_{f} |
| 979 | E₁ₐE₅E₄ | | | E_{f} |
| 980 | E_{1b}E₅E₄ | | | E_{f} |
| 981 | E₁ₐEᵢE₄ | | | E_{f} |
| 982 | E_{1b}EᵢE₄ | | | E_{f} |
| 983 | E₁ₐEᵢᵢE₄ | | | E_{f} |
| 984 | E_{1b}EᵢᵢE₄ | | | E_{f} |
| 985 | E₁ₐEᵢᵢE_{iib}E₄ | | | E_{f} |
| 986 | E_{1b}EᵢᵢE_{iib}E₄ | | | E_{f} |
| 987 | E₁ₐE₅EᵢE₄ | | | E_{f} |
| 988 | E_{1b}E₅EᵢE₄ | | | E_{f} |
| 989 | E₁ₐE₅EᵢᵢE₄ | | | E_{f} |
| 990 | E_{1b}E₅EᵢᵢE₄ | | | E_{f} |
| 991 | E₁ₐE₅EᵢᵢE_{iib}E₄ | | | E_{f} |
| 992 | E_{1b}E₅EᵢᵢE_{iib}E₄ | | | E_{f} |
| 993 | E₁ₐEᵢᵢᵢ | | | E_{f} |
| 994 | E_{1b}Eᵢᵢᵢ | | | E_{f} |
| 995 | E₁ₐE₅Eᵢᵢᵢ | | | E_{f} |
| 996 | E_{1b}E₅Eᵢᵢᵢ | | | E_{f} |
| 997 | E₁ₐEᵢᵢᵢE₄ | | | E_{f} |
| 998 | E_{1b}EᵢᵢᵢE₄ | | | E_{f} |
| 999 | E₁ₐE₅EᵢᵢᵢE₄ | | | E_{f} |
| 1000 | E_{1b}E₅EᵢᵢᵢE₄ | | | E_{f} |
| 1001 | E₁ₐEᵢᵥ | | | E_{f} |
| 1002 | E_{1b}Eᵢᵥ | | | E_{f} |
| 1003 | E₁ₐE₅Eᵢᵥ | | | E_{f} |
| 1004 | E_{1b}E₅Eᵢᵥ | | | E_{f} |
| 1005 | E₁ₐEᵢᵥE₄ | | | E_{f} |
| 1006 | E_{1b}EᵢᵥE₄ | | | E_{f} |
| 1007 | E₁ₐE₅EᵢᵥE₄ | | | E_{f} |
| 1008 | E_{1b}E₅EᵢᵥE₄ | | | E_{f} |
| 1009 | E₁ₐ | | E₄* | E_{f} |
| 1010 | E_{1b} | | E₄* | E_{f} |
| 1011 | E₁ₐE₅ | | E₄* | E_{f} |
| 1012 | E_{1b}E₅ | | E₄* | E_{f} |
| 1013 | E₁ₐEᵢ | | E₄* | E_{f} |
| 1014 | E_{1b}Eᵢ | | E₄* | E_{f} |
| 1015 | E₁ₐEᵢᵢ | | E₄* | E_{f} |
| 1016 | E_{1b}Eᵢᵢ | | E₄* | E_{f} |
| 1017 | E₁ₐEᵢᵢE_{iib} | | E₄* | E_{f} |
| 1018 | E_{1b}EᵢᵢE_{iib} | | E₄* | E_{f} |
| 1019 | E₁ₐE₅Eᵢ | | E₄* | E_{f} |
| 1020 | E_{1b}E₅Eᵢ | | E₄* | E_{f} |
| 1021 | E₁ₐE₅Eᵢᵢ | | E₄* | E_{f} |
| 1022 | E_{1b}E₅Eᵢᵢ | | E₄* | E_{f} |
| 1023 | E₁ₐE₅EᵢᵢE_{iib} | | E₄* | E_{f} |
| 1024 | E_{1b}E₅EᵢᵢE_{iib} | | E₄* | E_{f} |
| 1025 | E₁ₐE₄ | | E₄* | E_{f} |
| 1026 | E_{1b}E₄ | | E₄* | E_{f} |
| 1027 | E₁ₐE₅E₄ | | E₄* | E_{f} |
| 1028 | E_{1b}E₅E₄ | | E₄* | E_{f} |
| 1029 | E₁ₐEᵢE₄ | | E₄* | E_{f} |
| 1030 | E_{1b}EᵢE₄ | | E₄* | E_{f} |
| 1031 | E₁ₐEᵢᵢE₄ | | E₄* | E_{f} |
| 1032 | E_{1b}EᵢᵢE₄ | | E₄* | E_{f} |
| 1033 | E₁ₐEᵢᵢE_{iib}E₄ | | E₄* | E_{f} |
| 1034 | E_{1b}EᵢᵢE_{iib}E₄ | | E₄* | E_{f} |
| 1035 | E₁ₐE₅EᵢE₄ | | E₄* | E_{f} |
| 1036 | E_{1b}E₅EᵢE₄ | | E₄* | E_{f} |
| 1037 | E₁ₐE₅EᵢᵢE₄ | | E₄* | E_{f} |
| 1038 | E_{1b}E₅EᵢᵢE₄ | | E₄* | E_{f} |
| 1039 | E₁ₐE₅EᵢᵢE_{iib}E₄ | | E₄* | E_{f} |
| 1040 | E_{1b}E₅EᵢᵢE_{iib}E₄ | | E₄* | E_{f} |
| 1041 | E₁ₐEᵢᵢᵢ | | E₄* | E_{f} |
| 1042 | E_{1b}Eᵢᵢᵢ | | E₄* | E_{f} |
| 1043 | E₁ₐE₅Eᵢᵢᵢ | | E₄* | E_{f} |
| 1044 | E_{1b}E₅Eᵢᵢᵢ | | E₄* | E_{f} |
| 1045 | E₁ₐEᵢᵢᵢE₄ | | E₄* | E_{f} |
| 1046 | E_{1b}EᵢᵢᵢE₄ | | E₄* | E_{f} |
| 1047 | E₁ₐE₅EᵢᵢᵢE₄ | | E₄* | E_{f} |
| 1048 | E_{1b}E₅EᵢᵢᵢE₄ | | E₄* | E_{f} |
| 1049 | E₁ₐEᵢᵥ | | E₄* | E_{f} |
| 1050 | E_{1b}Eᵢᵥ | | E₄* | E_{f} |
| 1051 | E₁ₐE₅Eᵢᵥ | | E₄* | E_{f} |
| 1052 | E_{1b}E₅Eᵢᵥ | | E₄* | E_{f} |
| 1053 | E₁ₐEᵢᵥE₄ | | E₄* | E_{f} |
| 1054 | E_{1b}EᵢᵥE₄ | | E₄* | E_{f} |
| 1055 | E₁ₐE₅EᵢᵥE₄ | | E₄* | E_{f} |
| 1056 | E_{1b}E₅EᵢᵥE₄ | | E₄* | E_{f} |
| 1057 | E₁ₐ | AlkL | | E_{f} |
| 1058 | E_{1b} | AlkL | | E_{f} |
| 1059 | E₁ₐE₅ | AlkL | | E_{f} |
| 1060 | E_{1b}E₅ | AlkL | | E_{f} |
| 1061 | E₁ₐEᵢ | AlkL | | E_{f} |
| 1062 | E_{1b}Eᵢ | AlkL | | E_{f} |
| 1063 | E₁ₐEᵢᵢ | AlkL | | E_{f} |
| 1064 | E_{1b}Eᵢᵢ | AlkL | | E_{f} |
| 1065 | E₁ₐEᵢᵢE_{iib} | AlkL | | E_{f} |
| 1066 | E_{1b}EᵢᵢE_{iib} | AlkL | | E_{f} |
| 1067 | E₁ₐE₅Eᵢ | AlkL | | E_{f} |
| 1068 | E_{1b}E₅Eᵢ | AlkL | | E_{f} |
| 1069 | E₁ₐE₅Eᵢᵢ | AlkL | | E_{f} |
| 1070 | E_{1b}E₅Eᵢᵢ | AlkL | | E_{f} |
| 1071 | E₁ₐE₅EᵢᵢE_{iib} | AlkL | | E_{f} |
| 1072 | E_{1b}E₅EᵢᵢE_{iib} | AlkL | | E_{f} |
| 1073 | E₁ₐE₄ | AlkL | | E_{f} |
| 1074 | E_{1b}E₄ | AlkL | | E_{f} |
| 1075 | E₁ₐE₅E₄ | AlkL | | E_{f} |
| 1076 | E_{1b}E₅E₄ | AlkL | | E_{f} |
| 1077 | E₁ₐEᵢE₄ | AlkL | | E_{f} |
| 1078 | E_{1b}EᵢE₄ | AlkL | | E_{f} |
| 1079 | E₁ₐEᵢᵢE₄ | AlkL | | E_{f} |
| 1080 | E_{1b}EᵢᵢE₄ | AlkL | | E_{f} |
| 1081 | E₁ₐEᵢᵢE_{iib}E₄ | AlkL | | E_{f} |
| 1082 | E_{1b}EᵢᵢE_{iib}E₄ | AlkL | | E_{f} |
| 1083 | E₁ₐE₅EᵢE₄ | AlkL | | E_{f} |
| 1084 | E_{1b}E₅EᵢE₄ | AlkL | | E_{f} |
| 1085 | E₁ₐE₅EᵢᵢE₄ | AlkL | | E_{f} |
| 1086 | E_{1b}E₅EᵢᵢE₄ | AlkL | | E_{f} |
| 1087 | E₁ₐE₅EᵢᵢE_{iib}E₄ | AlkL | | E_{f} |
| 1088 | E_{1b}E₅EᵢᵢE_{iib}E₄ | AlkL | | E_{f} |
| 1089 | E₁ₐEᵢᵢᵢ | AlkL | | E_{f} |
| 1090 | E_{1b}Eᵢᵢᵢ | AlkL | | E_{f} |
| 1091 | E₁ₐE₅Eᵢᵢᵢ | AlkL | | E_{f} |
| 1092 | E_{1b}E₅Eᵢᵢᵢ | AlkL | | E_{f} |
| 1093 | E₁ₐEᵢᵢᵢE₄ | AlkL | | E_{f} |
| 1094 | E_{1b}EᵢᵢᵢE₄ | AlkL | | E_{f} |
| 1095 | E₁ₐE₅EᵢᵢᵢE₄ | AlkL | | E_{f} |
| 1096 | E_{1b}E₅EᵢᵢᵢE₄ | AlkL | | E_{f} |
| 1097 | E₁ₐEᵢᵥ | AlkL | | E_{f} |
| 1098 | E_{1b}Eᵢᵥ | AlkL | | E_{f} |
| 1099 | E₁ₐE₅Eᵢᵥ | AlkL | | E_{f} |
| 1100 | E_{1b}E₅Eᵢᵥ | AlkL | | E_{f} |
| 1101 | E₁ₐEᵢᵥE₄ | AlkL | | E_{f} |
| 1102 | E_{1b}EᵢᵥE₄ | AlkL | | E_{f} |
| 1103 | E₁ₐE₅EᵢᵥE₄ | AlkL | | E_{f} |
| 1104 | E_{1b}E₅EᵢᵥE₄ | AlkL | | E_{f} |
| 1105 | E₁ₐ | AlkL | E₄* | E_{f} |
| 1106 | E_{1b} | AlkL | E₄* | E_{f} |
| 1107 | E₁ₐE₅ | AlkL | E₄* | E_{f} |
| 1108 | E_{1b}E₅ | AlkL | E₄* | E_{f} |
| 1109 | E₁ₐEᵢ | AlkL | E₄* | E_{f} |
| 1110 | E_{1b}Eᵢ | AlkL | E₄* | E_{f} |
| 1111 | E₁ₐEᵢᵢ | AlkL | E₄* | E_{f} |
| 1112 | E_{1b}Eᵢᵢ | AlkL | E₄* | E_{f} |
| 1113 | E₁ₐEᵢᵢE_{iib} | AlkL | E₄* | E_{f} |
| 1114 | E_{1b}EᵢᵢE_{iib} | AlkL | E₄* | E_{f} |
| 1115 | E₁ₐE₅Eᵢ | AlkL | E₄* | E_{f} |
| 1116 | E_{1b}E₅Eᵢ | AlkL | E₄* | E_{f} |
| 1117 | E₁ₐE₅Eᵢᵢ | AlkL | E₄* | E_{f} |
| 1118 | E_{1b}E₅Eᵢᵢ | AlkL | E₄* | E_{f} |
| 1119 | E₁ₐE₅EᵢᵢE_{iib} | AlkL | E₄* | E_{f} |
| 1120 | E_{1b}E₅EᵢᵢE_{iib} | AlkL | E₄* | E_{f} |
| 1121 | E₁ₐE₄ | AlkL | E₄* | E_{f} |
| 1122 | E_{1b}E₄ | AlkL | E₄* | E_{f} |
| 1123 | E₁ₐE₅E₄ | AlkL | E₄* | E_{f} |
| 1124 | E_{1b}E₅E₄ | AlkL | E₄* | E_{f} |
| 1125 | E₁ₐEᵢE₄ | AlkL | E₄* | E_{f} |
| 1126 | E_{1b}EᵢE₄ | AlkL | E₄* | E_{f} |
| 1127 | E₁ₐEᵢᵢE₄ | AlkL | E₄* | E_{f} |
| 1128 | E_{1b}EᵢᵢE₄ | AlkL | E₄* | E_{f} |
| 1129 | E₁ₐEᵢᵢE_{iib}E₄ | AlkL | E₄* | E_{f} |
| 1130 | E_{1b}EᵢᵢE_{iib}E₄ | AlkL | E₄* | E_{f} |
| 1131 | E₁ₐE₅EᵢE₄ | AlkL | E₄* | E_{f} |
| 1132 | E_{1b}E₅EᵢE₄ | AlkL | E₄* | E_{f} |
| 1133 | E₁ₐE₅EᵢᵢE₄ | AlkL | E₄* | E_{f} |
| 1134 | E_{1b}E₅EᵢᵢE₄ | AlkL | E₄* | E_{f} |
| 1135 | E₁ₐE₅EᵢᵢE_{iib}E₄ | AlkL | E₄* | E_{f} |
| 1136 | E_{1b}E₅EᵢᵢE_{iib}E₄ | AlkL | E₄* | E_{f} |
| 1137 | E₁ₐEᵢᵢᵢ | AlkL | E₄* | E_{f} |
| 1138 | E_{1b}Eᵢᵢᵢ | AlkL | E₄* | E_{f} |
| 1139 | E₁ₐE₅Eᵢᵢᵢ | AlkL | E₄* | E_{f} |
| 1140 | E_{1b}E₅Eᵢᵢᵢ | AlkL | E₄* | E_{f} |
| 1141 | E₁ₐEᵢᵢᵢE₄ | AlkL | E₄* | E_{f} |
| 1142 | E_{1b}EᵢᵢᵢE₄ | AlkL | E₄* | E_{f} |
| 1143 | E₁ₐE₅EᵢᵢᵢE₄ | AlkL | E₄* | E_{f} |
| 1144 | E_{1b}E₅EᵢᵢᵢE₄ | AlkL | E₄* | E_{f} |
| 1145 | E₁ₐEᵢᵥ | AlkL | E₄* | E_{f} |
| 1146 | E_{1b}Eᵢᵥ | AlkL | E₄* | E_{f} |
| 1147 | E₁ₐE₅Eᵢᵥ | AlkL | E₄* | E_{f} |
| 1148 | E_{1b}E₅Eᵢᵥ | AlkL | E₄* | E_{f} |
| 1149 | E₁ₐEᵢᵥE₄ | AlkL | E₄* | E_{f} |
| 1150 | E_{1b}EᵢᵥE₄ | AlkL | E₄* | E_{f} |
| 1151 | E₁ₐE₅EᵢᵥE₄ | AlkL | E₄* | E_{f} |
| 1152 | E_{1b}E₅EᵢᵥE₄ | AlkL | E₄* | E_{f} |

Microorganisms quite especially preferred according to the invention (abbreviated as MO) are outstandingly suitable for the production of ω-carboxycarboxylate esters and have increased or decreased enzyme activities (abbreviated as E) described in the following table, where these can in addition advantageously be combined with an increased enzyme activity compared to the wild type of the microorganism, which is described for the 3-ketoacyl-ACP (Acyl Carrrier Protein) synthase III (EC 2.3.1.41), in particular that from plants, preferably that from plants the seeds whereof contain fatty acids with alkyl residues shorter than 14 C atoms, and particularly preferably that from plants of the genera Cuphea, Elaeis, Cocos, Umbellularia and Cinnamomum and gene products selected from AccA, AccB, AccC, AccD, AceE, AceF, Lpd, AcpP, FabA, FabB, FabD, FabF, FabG, FabH, Fabl, FabZ, PanD, PanK, UdhA, PntA or PntB.

Any combinations of at least two of these enzyme activities can advantageously be increased.

In addition, moreover, it can be advantageous if the microorganism is provided with a lower enzyme activity compared to the wild type of the microorganism, which is described for the gene products selected from TdcE, PflA, PflB, PflC, PflD, PoxB, YgfG, AckA, AckB, TdcD, Pta, LdhA, AdhE, MgsA, FdnG, FdnH, FdnI, FdhF, FdoG, FdoH, FdoI, PrpC, PrpD, PrpF, PrpB, TdcD, Pdc, PorA, PorB, PorC, PorD, AlsS, IlvB, IlvM, IlvN, IlvG, IlyI, IlvH, AlsD, ButB, Thl, ThlA, ThlB, PhaA, PhaB, Crt, BdhA, BdhB, Adc, Adh, CtfB, AtoA, AtoD, LdhL, GltA, FabR, FhuA, Dld, LldA or LldP, singly or in any combination.

| | | |
|---|---|---|
| MO | E increased | E decreased |

| | | | | |
|---|---|---|---|---|
| 1 | E₁ₐ | EᵥEᵥᵢ | | |
| 2 | E_{1b} | EᵥEᵥᵢ | | |
| 3 | E₁ₐE₅ | EᵥEᵥᵢ | | |
| 4 | E_{1b}E₅ | EᵥEᵥᵢ | | |
| 5 | E₁ₐEᵢ | EᵥEᵥᵢ | | |
| 6 | E_{1b}Eᵢ | EᵥEᵥᵢ | | |
| 7 | E₁ₐEᵢᵢ | EᵥEᵥᵢ | | |
| 8 | E_{1b}Eᵢᵢ | EᵥEᵥᵢ | | |
| 9 | E₁ₐEᵢᵢE_{iib} | EᵥEᵥᵢ | | |
| 10 | E_{1b}EᵢᵢE_{iib} | EᵥEᵥᵢ | | |
| 11 | E₁ₐE₅Eᵢ | EᵥEᵥᵢ | | |
| 12 | E_{1b}E₅Eᵢ | EᵥEᵥᵢ | | |
| 13 | E₁ₐE₅Eᵢᵢ | EᵥEᵥᵢ | | |
| 14 | E_{1b}E₅Eᵢᵢ | EᵥEᵥᵢ | | |
| 15 | E₁ₐE₅EᵢᵢE_{iib} | EᵥEᵥᵢ | | |
| 16 | E_{1b}E₅EᵢᵢE_{iib} | EᵥEᵥᵢ | | |
| 17 | E₁ₐE₄ | EᵥEᵥᵢ | | |
| 18 | E_{1b}E₄ | EᵥEᵥᵢ | | |
| 19 | E₁ₐE₅E₄ | EᵥEᵥᵢ | | |
| 20 | E_{1b}E₅E₄ | EᵥEᵥᵢ | | |
| 21 | E₁ₐEᵢE₄ | EᵥEᵥᵢ | | |
| 22 | E_{1b}EᵢE₄ | EᵥEᵥᵢ | | |
| 23 | E₁ₐEᵢᵢE₄ | EᵥEᵥᵢ | | |
| 24 | E_{1b}EᵢᵢE₄ | EᵥEᵥᵢ | | |
| 25 | E₁ₐEᵢᵢE_{iib}E₄ | EᵥEᵥᵢ | | |
| 26 | E_{1b}EᵢᵢE_{iib}E₄ | EᵥEᵥᵢ | | |
| 27 | E₁ₐE₅EᵢE₄ | EᵥEᵥᵢ | | |
| 28 | E_{1b}E₅EᵢE₄ | EᵥEᵥᵢ | | |
| 29 | E₁ₐE₅EᵢᵢE₄ | EᵥEᵥᵢ | | |
| 30 | E_{1b}E₅EᵢᵢE₄ | EᵥEᵥᵢ | | |
| 31 | E₁ₐE₅EᵢᵢE_{iib}E₄ | EᵥEᵥᵢ | | |
| 32 | E_{1b}E₅EᵢᵢE_{iib}E₄ | EᵥEᵥᵢ | | |
| 33 | E₁ₐEᵢᵢᵢ | EᵥEᵥᵢ | | |
| 34 | E_{1b}Eᵢᵢᵢ | EᵥEᵥᵢ | | |
| 35 | E₁ₐE₅Eᵢᵢᵢ | EᵥEᵥᵢ | | |
| 36 | E_{1b}E₅Eᵢᵢᵢ | EᵥEᵥᵢ | | |
| 37 | E₁ₐEᵢᵢᵢE₄ | EᵥEᵥᵢ | | |
| 38 | E_{1b}EᵢᵢᵢE₄ | EᵥEᵥᵢ | | |
| 39 | E₁ₐE₅EᵢᵢᵢE₄ | EᵥEᵥᵢ | | |
| 40 | E_{1b}E₅EᵢᵢᵢE₄ | EᵥEᵥᵢ | | |
| 41 | E₁ₐEᵢᵥ | EᵥEᵥᵢ | | |
| 42 | E_{1b}Eᵢᵥ | EᵥEᵥᵢ | | |
| 43 | E₁ₐE₅Eᵢᵥ | EᵥEᵥᵢ | | |
| 44 | E_{1b}E₅Eᵢᵥ | EᵥEᵥᵢ | | |
| 45 | E₁ₐEᵢᵥE₄ | EᵥEᵥᵢ | | |
| 46 | E_{1b}EᵢᵥE₄ | EᵥEᵥᵢ | | |
| 47 | E₁ₐE₅EᵢᵥE₄ | EᵥEᵥᵢ | | |
| 48 | E_{1b}E₅EᵢᵥE₄ | EᵥEᵥᵢ | | |
| 49 | E₁ₐ | EᵥEᵥᵢ | E₄* | |
| 50 | E_{1b} | EᵥEᵥᵢ | E₄* | |
| 51 | E₁ₐE₅ | EᵥEᵥᵢ | E₄* | |
| 52 | E_{1b}E₅ | EᵥEᵥᵢ | E₄* | |
| 53 | E₁ₐEᵢ | EᵥEᵥᵢ | E₄* | |
| 54 | E_{1b}Eᵢ | EᵥEᵥᵢ | E₄* | |
| 55 | E₁ₐEᵢᵢ | EᵥEᵥᵢ | E₄* | |
| 56 | E_{1b}Eᵢᵢ | EᵥEᵥᵢ | E₄* | |
| 57 | E₁ₐEᵢᵢE_{iib} | EᵥEᵥᵢ | E₄* | |
| 58 | E_{1b}EᵢᵢE_{iib} | EᵥEᵥᵢ | E₄* | |
| 59 | E₁ₐE₅Eᵢ | EᵥEᵥᵢ | E₄* | |
| 60 | E_{1b}E₅Eᵢ | EᵥEᵥᵢ | E₄* | |
| 61 | E₁ₐE₅Eᵢᵢ | EᵥEᵥᵢ | E₄* | |
| 62 | E_{1b}E₅Eᵢᵢ | EᵥEᵥᵢ | E₄* | |
| 63 | E₁ₐE₅EᵢᵢE_{iib} | EᵥEᵥᵢ | E₄* | |
| 64 | E_{1b}E₅EᵢᵢE_{iib} | EᵥEᵥᵢ | E₄* | |
| 65 | E₁ₐE₄ | EᵥEᵥᵢ | E₄* | |
| 66 | E_{1b}E₄ | EᵥEᵥᵢ | E₄* | |
| 67 | E₁ₐE₅E₄ | EᵥEᵥᵢ | E₄* | |
| 68 | E_{1b}E₅E₄ | EᵥEᵥᵢ | E₄* | |
| 69 | E₁ₐEᵢE₄ | EᵥEᵥᵢ | E₄* | |
| 70 | E_{1b}EᵢE₄ | EᵥEᵥᵢ | E₄* | |
| 71 | E₁ₐEᵢᵢE₄ | EᵥEᵥᵢ | E₄* | |
| 72 | E_{1b}EᵢᵢE₄ | EᵥEᵥᵢ | E₄* | |
| 73 | E₁ₐEᵢᵢE_{iib}E₄ | EᵥEᵥᵢ | E₄* | |
| 74 | E_{1b}EᵢᵢE_{iib}E₄ | EᵥEᵥᵢ | E₄* | |
| 75 | E₁ₐE₅EᵢE₄ | EᵥEᵥᵢ | E₄* | |
| 76 | E_{1b}E₅EᵢE₄ | EᵥEᵥᵢ | E₄* | |
| 77 | E₁ₐE₅EᵢᵢE₄ | EᵥEᵥᵢ | E₄* | |
| 78 | E_{1b}E₅EᵢᵢE₄ | EᵥEᵥᵢ | E₄* | |
| 79 | E₁ₐE₅EᵢᵢE_{iib}E₄ | EᵥEᵥᵢ | E₄* | |
| 80 | E_{1b}E₅EᵢᵢE_{iib}E₄ | EᵥEᵥᵢ | E₄* | |
| 81 | E₁ₐEᵢᵢᵢ | EᵥEᵥᵢ | E₄* | |
| 82 | E_{1b}Eᵢᵢᵢ | EᵥEᵥᵢ | E₄* | |
| 83 | E₁ₐE₅Eᵢᵢᵢ | EᵥEᵥᵢ | E₄* | |
| 84 | E_{1b}E₅Eᵢᵢᵢ | EᵥEᵥᵢ | E₄* | |
| 85 | E₁ₐEᵢᵢᵢE₄ | EᵥEᵥᵢ | E₄* | |
| 86 | E_{1b}EᵢᵢᵢE₄ | EᵥEᵥᵢ | E₄* | |
| 87 | E₁ₐE₅EᵢᵢᵢE₄ | EᵥEᵥᵢ | E₄* | |
| 88 | E_{1b}E₅EᵢᵢᵢE₄ | EᵥEᵥᵢ | E₄* | |
| 89 | E₁ₐEᵢᵥ | EᵥEᵥᵢ | E₄* | |
| 90 | E_{1b}Eᵢᵥ | EᵥEᵥᵢ | E₄* | |
| 91 | E₁ₐE₅Eᵢᵥ | EᵥEᵥᵢ | E₄* | |
| 92 | E_{1b}E₅Eᵢᵥ | EᵥEᵥᵢ | E₄* | |
| 93 | E₁ₐEᵢᵥE₄ | EᵥEᵥᵢ | E₄* | |
| 94 | E_{1b}EᵢᵥE₄ | EᵥEᵥᵢ | E₄* | |
| 95 | E₁ₐE₅EᵢᵥE₄ | EᵥEᵥᵢ | E₄* | |
| 96 | E_{1b}E₅EᵢᵥE₄ | EᵥEᵥᵢ | E₄* | |
| 97 | E₁ₐ | AlkLEᵥEᵥᵢ | | |
| 98 | E_{1b} | AlkLEᵥEᵥᵢ | | |
| 99 | E₁ₐE₅ | AlkLEᵥEᵥᵢ | | |
| 100 | E_{1b}E₅ | AlkLEᵥEᵥᵢ | | |
| 101 | E₁ₐEᵢ | AlkLEᵥEᵥᵢ | | |
| 102 | E_{1b}Eᵢ | AlkLEᵥEᵥᵢ | | |
| 103 | E₁ₐEᵢᵢ | AlkLEᵥEᵥᵢ | | |
| 104 | E_{1b}Eᵢᵢ | AlkLEᵥEᵥᵢ | | |
| 105 | E₁ₐEᵢᵢE_{iib} | AlkLEᵥEᵥᵢ | | |
| 106 | E_{1b}EᵢᵢE_{iib} | AlkLEᵥEᵥᵢ | | |
| 107 | E₁ₐE₅Eᵢ | AlkLEᵥEᵥᵢ | | |
| 108 | E_{1b}E₅Eᵢ | AlkLEᵥEᵥᵢ | | |
| 109 | E₁ₐE₅Eᵢᵢ | AlkLEᵥEᵥᵢ | | |
| 110 | E_{1b}E₅Eᵢᵢ | AlkLEᵥEᵥᵢ | | |
| 111 | E₁ₐE₅EᵢᵢE_{iib} | AlkLEᵥEᵥᵢ | | |
| 112 | E_{1b}E₅EᵢᵢE_{iib} | AlkLEᵥEᵥᵢ | | |
| 113 | E₁ₐE₄ | AlkLEᵥEᵥᵢ | | |
| 114 | E_{1b}E₄ | AlkLEᵥEᵥᵢ | | |
| 115 | E₁ₐE₅E₄ | AlkLEᵥEᵥᵢ | | |
| 116 | E_{1b}E₅E₄ | AlkLEᵥEᵥᵢ | | |
| 117 | E₁ₐEᵢE₄ | AlkLEᵥEᵥᵢ | | |
| 118 | E_{1b}EᵢE₄ | AlkLEᵥEᵥᵢ | | |
| 119 | E₁ₐEᵢᵢE₄ | AlkLEᵥEᵥᵢ | | |
| 120 | E_{1b}EᵢᵢE₄ | AlkLEᵥEᵥᵢ | | |
| 121 | E₁ₐEᵢᵢE_{iib}E₄ | AlkLEᵥEᵥᵢ | | |
| 122 | E_{1b}EᵢᵢE_{iib}E₄ | AlkLEᵥEᵥᵢ | | |
| 123 | E₁ₐE₅EᵢE₄ | AlkLEᵥEᵥᵢ | | |
| 124 | E_{1b}E₅EᵢE₄ | AlkLEᵥEᵥᵢ | | |
| 125 | E₁ₐE₅EᵢᵢE₄ | AlkLEᵥEᵥᵢ | | |
| 126 | E_{1b}E₅EᵢᵢE₄ | AlkLEᵥEᵥᵢ | | |
| 127 | E₁ₐE₅EᵢᵢE_{iib}E₄ | AlkLEᵥEᵥᵢ | | |
| 128 | E_{1b}E₅EᵢᵢE_{iib}E₄ | AlkLEᵥEᵥᵢ | | |
| 129 | E₁ₐEᵢᵢᵢ | AlkLEᵥEᵥᵢ | | |
| 130 | E_{1b}Eᵢᵢᵢ | AlkLEᵥEᵥᵢ | | |
| 131 | E₁ₐE₅Eᵢᵢᵢ | AlkLEᵥEᵥᵢ | | |
| 132 | E_{1b}E₅Eᵢᵢᵢ | AlkLEᵥEᵥᵢ | | |
| 133 | E₁ₐEᵢᵢᵢE₄ | AlkLEᵥEᵥᵢ | | |
| 134 | E_{1b}EᵢᵢᵢE₄ | AlkLEᵥEᵥᵢ | | |
| 135 | E₁ₐE₅EᵢᵢᵢE₄ | AlkLEᵥEᵥᵢ | | |
| 136 | E_{1b}E₅EᵢᵢᵢE₄ | AlkLEᵥEᵥᵢ | | |
| 137 | E₁ₐEᵢᵥ | AlkLEᵥEᵥᵢ | | |
| 138 | E_{1b}Eᵢᵥ | AlkLEᵥEᵥᵢ | | |
| 139 | E₁ₐE₅Eᵢᵥ | AlkLEᵥEᵥᵢ | | |
| 140 | E_{1b}E₅Eᵢᵥ | AlkLEᵥEᵥᵢ | | |
| 141 | E₁ₐEᵢᵥE₄ | AlkLEᵥEᵥᵢ | | |
| 142 | E_{1b}EᵢᵥE₄ | AlkLEᵥEᵥᵢ | | |
| 143 | E₁ₐE₅EᵢᵥE₄ | AlkLEᵥEᵥᵢ | | |
| 144 | E_{1b}E₅EᵢᵥE₄ | AlkLEᵥEᵥᵢ | | |
| 145 | E₁ₐ | AlkLEᵥEᵥᵢ | E₄* | |
| 146 | E_{1b} | AlkLEᵥEᵥᵢ | E₄* | |
| 147 | E₁ₐE₅ | AlkLEᵥEᵥᵢ | E₄* | |
| 148 | E_{1b}E₅ | AlkLEᵥEᵥᵢ | E₄* | |
| 149 | E₁ₐEᵢ | AlkLEᵥEᵥᵢ | E₄* | |
| 150 | E_{1b}Eᵢ | AlkLEᵥEᵥᵢ | E₄* | |
| 151 | E₁ₐEᵢᵢ | AlkLEᵥEᵥᵢ | E₄* | |
| 152 | E_{1b}Eᵢᵢ | AlkLEᵥEᵥᵢ | E₄* | |
| 153 | E₁ₐEᵢᵢE_{iib} | AlkLEᵥEᵥᵢ | E₄* | |
| 154 | E_{1b}EᵢᵢE_{iib} | AlkLEᵥEᵥᵢ | E₄* | |
| 155 | E₁ₐE₅Eᵢ | AlkLEᵥEᵥᵢ | E₄* | |
| 156 | E_{1b}E₅Eᵢ | AlkLEᵥEᵥᵢ | E₄* | |
| 157 | E₁ₐE₅Eᵢᵢ | AlkLEᵥEᵥᵢ | E₄* | |
| 158 | E_{1b}E₅Eᵢᵢ | AlkLEᵥEᵥᵢ | E₄* | |
| 159 | E₁ₐE₅EᵢᵢE_{iib} | AlkLEᵥEᵥᵢ | E₄* | |
| 160 | E_{1b}E₅EᵢᵢE_{iib} | AlkLEᵥEᵥᵢ | E₄* | |
| 161 | E₁ₐE₄ | AlkLEᵥEᵥᵢ | E₄* | |
| 162 | E_{1b}E₄ | AlkLEᵥEᵥᵢ | E₄* | |
| 163 | E₁ₐE₅E₄ | AlkLEᵥEᵥᵢ | E₄* | |
| 164 | E_{1b}E₅E₄ | AlkLEᵥEᵥᵢ | E₄* | |
| 165 | E₁ₐEᵢE₄ | AlkLEᵥEᵥᵢ | E₄* | |
| 166 | E_{1b}EᵢE₄ | AlkLEᵥEᵥᵢ | E₄* | |
| 167 | E₁ₐEᵢᵢE₄ | AlkLEᵥEᵥᵢ | E₄* | |
| 168 | E_{1b}EᵢᵢE₄ | AlkLEᵥEᵥᵢ | E₄* | |
| 169 | E₁ₐEᵢᵢE_{iib}E₄ | AlkLEᵥEᵥᵢ | E₄* | |
| 170 | E_{1b}EᵢᵢE_{iib}E₄ | AlkLEᵥEᵥᵢ | E₄* | |
| 171 | E₁ₐE₅EᵢE₄ | AlkLEᵥEᵥᵢ | E₄* | |
| 172 | E_{1b}E₅EᵢE₄ | AlkLEᵥEᵥᵢ | E₄* | |
| 173 | E₁ₐE₅EᵢᵢE₄ | AlkLEᵥEᵥᵢ | E₄* | |
| 174 | E_{1b}E₅EᵢᵢE₄ | AlkLEᵥEᵥᵢ | E₄* | |
| 175 | E₁ₐE₅EᵢᵢE_{iib}E₄ | AlkLEᵥEᵥᵢ | E₄* | |
| 176 | E_{1b}E₅EᵢᵢE_{iib}E₄ | AlkLEᵥEᵥᵢ | E₄* | |
| 177 | E₁ₐEᵢᵢᵢ | AlkLEᵥEᵥᵢ | E₄* | |
| 178 | E_{1b}Eᵢᵢᵢ | AlkLEᵥEᵥᵢ | E₄* | |
| 179 | E₁ₐE₅Eᵢᵢᵢ | AlkLEᵥEᵥᵢ | E₄* | |
| 180 | E_{1b}E₅Eᵢᵢᵢ | AlkLEᵥEᵥᵢ | E₄* | |
| 181 | E₁ₐEᵢᵢᵢE₄ | AlkLEᵥEᵥᵢ | E₄* | |
| 182 | E_{1b}EᵢᵢᵢE₄ | AlkLEᵥEᵥᵢ | E₄* | |
| 183 | E₁ₐE₅EᵢᵢᵢE₄ | AlkLEᵥEᵥᵢ | E₄* | |
| 184 | E_{1b}E₅EᵢᵢᵢE₄ | AlkLEᵥEᵥᵢ | E₄* | |
| 185 | E₁ₐEᵢᵥ | AlkLEᵥEᵥᵢ | E₄* | |
| 186 | E_{1b}Eᵢᵥ | AlkLEᵥEᵥᵢ | E₄* | |
| 187 | E₁ₐE₅Eᵢᵥ | AlkLEᵥEᵥᵢ | E₄* | |
| 188 | E_{1b}E₅Eᵢᵥ | AlkLEᵥEᵥᵢ | E₄* | |
| 189 | E₁ₐEᵢᵥE₄ | AlkLEᵥEᵥᵢ | E₄* | |
| 190 | E_{1b}EᵢᵥE₄ | AlkLEᵥEᵥᵢ | E₄* | |
| 191 | E₁ₐE₅EᵢᵥE₄ | AlkLEᵥEᵥᵢ | E₄* | |
| 192 | E_{1b}E₅EᵢᵥE₄ | AlkLEᵥEᵥᵢ | E₄* | |
| 193 | E₁ₐ | EᵥEᵥᵢ | | Eₐ |
| 194 | E_{1b} | EᵥEᵥᵢ | | Eₐ |
| 195 | E₁ₐE₅ | EᵥEᵥᵢ | | Eₐ |
| 196 | E_{1b}E₅ | EᵥEᵥᵢ | | Eₐ |
| 197 | E₁ₐEᵢ | EᵥEᵥᵢ | | Eₐ |
| 198 | E_{1b}Eᵢ | EᵥEᵥᵢ | | Eₐ |
| 199 | E₁ₐEᵢᵢ | EᵥEᵥᵢ | | Eₐ |
| 200 | E_{1b}Eᵢᵢ | EᵥEᵥᵢ | | Eₐ |
| 201 | E₁ₐEᵢᵢE_{iib} | EᵥEᵥᵢ | | Eₐ |
| 202 | E_{1b}EᵢᵢE_{iib} | EᵥEᵥᵢ | | Eₐ |
| 203 | E₁ₐE₅Eᵢ | EᵥEᵥᵢ | | Eₐ |
| 204 | E_{1b}E₅Eᵢ | EᵥEᵥᵢ | | Eₐ |
| 205 | E₁ₐE₅Eᵢᵢ | EᵥEᵥᵢ | | Eₐ |
| 206 | E_{1b}E₅Eᵢᵢ | EᵥEᵥᵢ | | Eₐ |
| 207 | E₁ₐE₅EᵢᵢE_{iib} | EᵥEᵥᵢ | | Eₐ |
| 208 | E_{1b}E₅EᵢᵢE_{iib} | EᵥEᵥᵢ | | Eₐ |
| 209 | E₁ₐE₄ | EᵥEᵥᵢ | | Eₐ |
| 210 | E_{1b}E₄ | EᵥEᵥᵢ | | Eₐ |
| 211 | E₁ₐE₅E₄ | EᵥEᵥᵢ | | Eₐ |
| 212 | E_{1b}E₅E₄ | EᵥEᵥᵢ | | Eₐ |
| 213 | E₁ₐEᵢE₄ | EᵥEᵥᵢ | | Eₐ |
| 214 | E_{1b}EᵢE₄ | EᵥEᵥᵢ | | Eₐ |
| 215 | E₁ₐEᵢᵢE₄ | EᵥEᵥᵢ | | Eₐ |
| 216 | E_{1b}EᵢᵢE₄ | EᵥEᵥᵢ | | Eₐ |
| 217 | E₁ₐEᵢᵢE_{iib}E₄ | EᵥEᵥᵢ | | Eₐ |
| 218 | E_{1b}EᵢᵢE_{iib}E₄ | EᵥEᵥᵢ | | Eₐ |
| 219 | E₁ₐE₅EᵢE₄ | EᵥEᵥᵢ | | Eₐ |
| 220 | E_{1b}E₅EᵢE₄ | EᵥEᵥᵢ | | Eₐ |
| 221 | E₁ₐE₅EᵢᵢE₄ | EᵥEᵥᵢ | | Eₐ |
| 222 | E_{1b}E₅EᵢᵢE₄ | EᵥEᵥᵢ | | Eₐ |
| 223 | E₁ₐE₅EᵢᵢE_{iib}E₄ | EᵥEᵥᵢ | | Eₐ |
| 224 | E_{1b}E₅EᵢᵢE_{iib}E₄ | EᵥEᵥᵢ | | Eₐ |
| 225 | E₁ₐEᵢᵢᵢ | EᵥEᵥᵢ | | Eₐ |
| 226 | E_{1b}Eᵢᵢᵢ | EᵥEᵥᵢ | | Eₐ |
| 227 | E₁ₐE₅Eᵢᵢᵢ | EᵥEᵥᵢ | | Eₐ |
| 228 | E_{1b}E₅Eᵢᵢᵢ | EᵥEᵥᵢ | | Eₐ |
| 229 | E₁ₐEᵢᵢᵢE₄ | EᵥEᵥᵢ | | Eₐ |
| 230 | E_{1b}EᵢᵢᵢE₄ | EᵥEᵥᵢ | | Eₐ |
| 231 | E₁ₐE₅EᵢᵢᵢE₄ | EᵥEᵥᵢ | | Eₐ |
| 232 | E_{1b}E₅EᵢᵢᵢE₄ | EᵥEᵥᵢ | | Eₐ |
| 233 | E₁ₐEᵢᵥ | EᵥEᵥᵢ | | Eₐ |
| 234 | E_{1b}Eᵢᵥ | EᵥEᵥᵢ | | Eₐ |
| 235 | E₁ₐE₅Eᵢᵥ | EᵥEᵥᵢ | | Eₐ |
| 236 | E_{1b}E₅Eᵢᵥ | EᵥEᵥᵢ | | Eₐ |
| 237 | E₁ₐEᵢᵥE₄ | EᵥEᵥᵢ | | Eₐ |
| 238 | E_{1b}EᵢᵥE₄ | EᵥEᵥᵢ | | Eₐ |
| 239 | E₁ₐE₅EᵢᵥE₄ | EᵥEᵥᵢ | | Eₐ |
| 240 | E_{1b}E₅EᵢᵥE₄ | EᵥEᵥᵢ | | Eₐ |
| 241 | E₁ₐ | EᵥEᵥᵢ | E₄* | Eₐ |
| 242 | E_{1b} | EᵥEᵥᵢ | E₄* | Eₐ |
| 243 | E₁ₐE₅ | EᵥEᵥᵢ | E₄* | Eₐ |
| 244 | E_{1b}E₅ | EᵥEᵥᵢ | E₄* | Eₐ |
| 245 | E₁ₐEᵢ | EᵥEᵥᵢ | E₄* | Eₐ |
| 246 | E_{1b}Eᵢ | EᵥEᵥᵢ | E₄* | Eₐ |
| 247 | E₁ₐEᵢᵢ | EᵥEᵥᵢ | E₄* | Eₐ |
| 248 | E_{1b}Eᵢᵢ | EᵥEᵥᵢ | E₄* | Eₐ |
| 249 | E₁ₐEᵢᵢE_{iib} | EᵥEᵥᵢ | E₄* | Eₐ |
| 250 | E_{1b}EᵢᵢE_{iib} | EᵥEᵥᵢ | E₄* | Eₐ |
| 251 | E₁ₐE₅Eᵢ | EᵥEᵥᵢ | E₄* | Eₐ |
| 252 | E_{1b}E₅Eᵢ | EᵥEᵥᵢ | E₄* | Eₐ |
| 253 | E₁ₐE₅Eᵢᵢ | EᵥEᵥᵢ | E₄* | Eₐ |
| 254 | E_{1b}E₅Eᵢᵢ | EᵥEᵥᵢ | E₄* | Eₐ |
| 255 | E₁ₐE₅EᵢᵢE_{iib} | EᵥEᵥᵢ | E₄* | Eₐ |
| 256 | E_{1b}E₅EᵢᵢE_{iib} | EᵥEᵥᵢ | E₄* | Eₐ |
| 257 | E₁ₐE₄ | EᵥEᵥᵢ | E₄* | Eₐ |
| 258 | E_{1b}E₄ | EᵥEᵥᵢ | E₄* | Eₐ |
| 259 | E₁ₐE₅E₄ | EᵥEᵥᵢ | E₄* | Eₐ |
| 260 | E_{1b}E₅E₄ | EᵥEᵥᵢ | E₄* | Eₐ |
| 261 | E₁ₐEᵢE₄ | EᵥEᵥᵢ | E₄* | Eₐ |
| 262 | E_{1b}EᵢE₄ | EᵥEᵥᵢ | E₄* | Eₐ |
| 263 | E₁ₐEᵢᵢE₄ | EᵥEᵥᵢ | E₄* | Eₐ |
| 264 | E_{1b}EᵢᵢE₄ | EᵥEᵥᵢ | E₄* | Eₐ |
| 265 | E₁ₐEᵢᵢE_{iib}E₄ | EᵥEᵥᵢ | E₄* | Eₐ |
| 266 | E_{1b}EᵢᵢE_{iib}E₄ | EᵥEᵥᵢ | E₄* | Eₐ |
| 267 | E₁ₐE₅EᵢE₄ | EᵥEᵥᵢ | E₄* | Eₐ |
| 268 | E_{1b}E₅EᵢE₄ | EᵥEᵥᵢ | E₄* | Eₐ |
| 269 | E₁ₐE₅EᵢᵢE₄ | EᵥEᵥᵢ | E₄* | Eₐ |
| 270 | E_{1b}E₅EᵢᵢE₄ | EᵥEᵥᵢ | E₄* | Eₐ |
| 271 | E₁ₐE₅EᵢᵢE_{iib}E₄ | EᵥEᵥᵢ | E₄* | Eₐ |
| 272 | E_{1b}E₅EᵢᵢE_{iib}E₄ | EᵥEᵥᵢ | E₄* | Eₐ |
| 273 | E₁ₐEᵢᵢᵢ | EᵥEᵥᵢ | E₄* | Eₐ |
| 274 | E_{1b}Eᵢᵢᵢ | EᵥEᵥᵢ | E₄* | Eₐ |
| 275 | E₁ₐE₅Eᵢᵢᵢ | EᵥEᵥᵢ | E₄* | Eₐ |
| 276 | E_{1b}E₅Eᵢᵢᵢ | EᵥEᵥᵢ | E₄* | Eₐ |
| 277 | E₁ₐEᵢᵢᵢE₄ | EᵥEᵥᵢ | E₄* | Eₐ |
| 278 | E_{1b}EᵢᵢᵢE₄ | EᵥEᵥᵢ | E₄* | Eₐ |
| 279 | E₁ₐE₅EᵢᵢᵢE₄ | EᵥEᵥᵢ | E₄* | Eₐ |
| 280 | E_{1b}E₅EᵢᵢᵢE₄ | EᵥEᵥᵢ | E₄* | Eₐ |
| 281 | E₁ₐEᵢᵥ | EᵥEᵥᵢ | E₄* | Eₐ |
| 282 | E_{1b}Eᵢᵥ | EᵥEᵥᵢ | E₄* | Eₐ |
| 283 | E₁ₐE₅Eᵢᵥ | EᵥEᵥᵢ | E₄* | Eₐ |
| 284 | E_{1b}E₅Eᵢᵥ | EᵥEᵥᵢ | E₄* | Eₐ |
| 285 | E₁ₐEᵢᵥE₄ | EᵥEᵥᵢ | E₄* | Eₐ |
| 286 | E_{1b}EᵢᵥE₄ | EᵥEᵥᵢ | E₄* | Eₐ |
| 287 | E₁ₐE₅EᵢᵥE₄ | EᵥEᵥᵢ | E₄* | Eₐ |
| 288 | E_{1b}E₅EᵢᵥE₄ | EᵥEᵥᵢ | E₄* | Eₐ |
| 289 | E₁ₐ | AlkLEᵥEᵥᵢ | | Eₐ |
| 290 | E_{1b} | AlkLEᵥEᵥᵢ | | Eₐ |
| 291 | E₁ₐE₅ | AlkLEᵥEᵥᵢ | | Eₐ |
| 292 | E_{1b}E₅ | AlkLEᵥEᵥᵢ | | Eₐ |
| 293 | E₁ₐEᵢ | AlkLEᵥEᵥᵢ | | Eₐ |
| 294 | E_{1b}Eᵢ | AlkLEᵥEᵥᵢ | | Eₐ |
| 295 | E₁ₐEᵢᵢ | AlkLEᵥEᵥᵢ | | Eₐ |
| 296 | E_{1b}Eᵢᵢ | AlkLEᵥEᵥᵢ | | Eₐ |
| 297 | E₁ₐEᵢᵢE_{iib} | AlkLEᵥEᵥᵢ | | Eₐ |
| 298 | E_{1b}EᵢᵢE_{iib} | AlkLEᵥEᵥᵢ | | Eₐ |
| 299 | E₁ₐE₅Eᵢ | AlkLEᵥEᵥᵢ | | Eₐ |
| 300 | E_{1b}E₅Eᵢ | AlkLEᵥEᵥᵢ | | Eₐ |
| 301 | E₁ₐE₅Eᵢᵢ | AlkLEᵥEᵥᵢ | | Eₐ |
| 302 | E_{1b}E₅Eᵢᵢ | AlkLEᵥEᵥᵢ | | Eₐ |
| 303 | E₁ₐE₅EᵢᵢE_{iib} | AlkLEᵥEᵥᵢ | | Eₐ |
| 304 | E_{1b}E₅EᵢᵢE_{iib} | AlkLEᵥEᵥᵢ | | Eₐ |
| 305 | E₁ₐE₄ | AlkLEᵥEᵥᵢ | | Eₐ |
| 306 | E_{1b}E₄ | AlkLEᵥEᵥᵢ | | Eₐ |
| 307 | E₁ₐE₅E₄ | AlkLEᵥEᵥᵢ | | Eₐ |
| 308 | E_{1b}E₅E₄ | AlkLEᵥEᵥᵢ | | Eₐ |
| 309 | E₁ₐEᵢE₄ | AlkLEᵥEᵥᵢ | | Eₐ |
| 310 | E_{1b}EᵢE₄ | AlkLEᵥEᵥᵢ | | Eₐ |
| 311 | E₁ₐEᵢᵢE₄ | AlkLEᵥEᵥᵢ | | Eₐ |
| 312 | E_{1b}EᵢᵢE₄ | AlkLEᵥEᵥᵢ | | Eₐ |
| 313 | E₁ₐEᵢᵢE_{iib}E₄ | AlkLEᵥEᵥᵢ | | Eₐ |
| 314 | E_{1b}EᵢᵢE_{iib}E₄ | AlkLEᵥEᵥᵢ | | Eₐ |
| 315 | E₁ₐE₅EᵢE₄ | AlkLEᵥEᵥᵢ | | Eₐ |
| 316 | E_{1b}E₅EᵢE₄ | AlkLEᵥEᵥᵢ | | Eₐ |
| 317 | E₁ₐE₅EᵢᵢE₄ | AlkLEᵥEᵥᵢ | | Eₐ |
| 318 | E_{1b}E₅EᵢᵢE₄ | AlkLEᵥEᵥᵢ | | Eₐ |
| 319 | E₁ₐE₅EᵢᵢE_{iib}E₄ | AlkLEᵥEᵥᵢ | | Eₐ |
| 320 | E_{1b}E₅EᵢᵢE_{iib}E₄ | AlkLEᵥEᵥᵢ | | Eₐ |
| 321 | E₁ₐEᵢᵢᵢ | AlkLEᵥEᵥᵢ | | Eₐ |
| 322 | E_{1b}Eᵢᵢᵢ | AlkLEᵥEᵥᵢ | | Eₐ |
| 323 | E₁ₐE₅Eᵢᵢᵢ | AlkLEᵥEᵥᵢ | | Eₐ |
| 324 | E_{1b}E₅Eᵢᵢᵢ | AlkLEᵥEᵥᵢ | | Eₐ |
| 325 | E₁ₐEᵢᵢᵢE₄ | AlkLEᵥEᵥᵢ | | Eₐ |
| 326 | E_{1b}EᵢᵢᵢE₄ | AlkLEᵥEᵥᵢ | | Eₐ |
| 327 | E₁ₐE₅EᵢᵢᵢE₄ | AlkLEᵥEᵥᵢ | | Eₐ |
| 328 | E_{1b}E₅EᵢᵢᵢE₄ | AlkLEᵥEᵥᵢ | | Eₐ |
| 329 | E₁ₐEᵢᵥ | AlkLEᵥEᵥᵢ | | Eₐ |
| 330 | E_{1b}Eᵢᵥ | AlkLEᵥEᵥᵢ | | Eₐ |
| 331 | E₁ₐE₅Eᵢᵥ | AlkLEᵥEᵥᵢ | | Eₐ |
| 332 | E_{1b}E₅Eᵢᵥ | AlkLEᵥEᵥᵢ | | Eₐ |
| 333 | E₁ₐEᵢᵥE₄ | AlkLEᵥEᵥᵢ | | Eₐ |
| 334 | E_{1b}EᵢᵥE₄ | AlkLEᵥEᵥᵢ | | Eₐ |
| 335 | E₁ₐE₅EᵢᵥE₄ | AlkLEᵥEᵥᵢ | | Eₐ |
| 336 | E_{1b}E₅EᵢᵥE₄ | AlkLEᵥEᵥᵢ | | Eₐ |
| 337 | E₁ₐ | AlkLEᵥEᵥᵢ | E₄* | Eₐ |
| 338 | E_{1b} | AlkLEᵥEᵥᵢ | E₄* | Eₐ |
| 339 | E₁ₐE₅ | AlkLEᵥEᵥᵢ | E₄* | Eₐ |
| 340 | E_{1b}E₅ | AlkLEᵥEᵥᵢ | E₄* | Eₐ |
| 341 | E₁ₐEᵢ | AlkLEᵥEᵥᵢ | E₄* | Eₐ |
| 342 | E_{1b}Eᵢ | AlkLEᵥEᵥᵢ | E₄* | Eₐ |
| 343 | E₁ₐEᵢᵢ | AlkLEᵥEᵥᵢ | E₄* | Eₐ |
| 344 | E_{1b}Eᵢᵢ | AlkLEᵥEᵥᵢ | E₄* | Eₐ |
| 345 | E₁ₐEᵢᵢE_{iib} | AlkLEᵥEᵥᵢ | E₄* | Eₐ |
| 346 | E_{1b}EᵢᵢE_{iib} | AlkLEᵥEᵥᵢ | E₄* | Eₐ |
| 347 | E₁ₐE₅Eᵢ | AlkLEᵥEᵥᵢ | E₄* | Eₐ |
| 348 | E_{1b}E₅Eᵢ | AlkLEᵥEᵥᵢ | E₄* | Eₐ |
| 349 | E₁ₐE₅Eᵢᵢ | AlkLEᵥEᵥᵢ | E₄* | Eₐ |
| 350 | E_{1b}E₅Eᵢᵢ | AlkLEᵥEᵥᵢ | E₄* | Eₐ |
| 351 | E₁ₐE₅EᵢᵢE_{iib} | AlkLEᵥEᵥᵢ | E₄* | Eₐ |
| 352 | E_{1b}E₅EᵢᵢE_{iib} | AlkLEᵥEᵥᵢ | E₄* | Eₐ |
| 353 | E₁ₐE₄ | AlkLEᵥEᵥᵢ | E₄* | Eₐ |
| 354 | E_{1b}E₄ | AlkLEᵥEᵥᵢ | E₄* | Eₐ |
| 355 | E₁ₐE₅E₄ | AlkLEᵥEᵥᵢ | E₄* | Eₐ |
| 356 | E_{1b}E₅E₄ | AlkLEᵥEᵥᵢ | E₄* | Eₐ |
| 357 | E₁ₐEᵢE₄ | AlkLEᵥEᵥᵢ | E₄* | Eₐ |
| 358 | E_{1b}EᵢE₄ | AlkLEᵥEᵥᵢ | E₄* | Eₐ |
| 359 | E₁ₐEᵢᵢE₄ | AlkLEᵥEᵥᵢ | E₄* | Eₐ |
| 360 | E_{1b}EᵢᵢE₄ | AlkLEᵥEᵥᵢ | E₄* | Eₐ |
| 361 | E₁ₐEᵢᵢE_{iib}E₄ | AlkLEᵥEᵥᵢ | E₄* | Eₐ |
| 362 | E_{1b}EᵢᵢE_{iib}E₄ | AlkLEᵥEᵥᵢ | E₄* | Eₐ |
| 363 | E₁ₐE₅EᵢE₄ | AlkLEᵥEᵥᵢ | E₄* | Eₐ |
| 364 | E_{1b}E₅EᵢE₄ | AlkLEᵥEᵥᵢ | E₄* | Eₐ |
| 365 | E₁ₐE₅EᵢᵢE₄ | AlkLEᵥEᵥᵢ | E₄* | Eₐ |
| 366 | E_{1b}E₅EᵢᵢE₄ | AlkLEᵥEᵥᵢ | E₄* | Eₐ |
| 367 | E₁ₐE₅EᵢᵢE_{iib}E₄ | AlkLEᵥEᵥᵢ | E₄* | Eₐ |
| 368 | E_{1b}E₅EᵢᵢE_{iib}E₄ | AlkLEᵥEᵥᵢ | E₄* | Eₐ |
| 369 | E₁ₐEᵢᵢᵢ | AlkLEᵥEᵥᵢ | E₄* | Eₐ |
| 370 | E_{1b}Eᵢᵢᵢ | AlkLEᵥEᵥᵢ | E₄* | Eₐ |
| 371 | E₁ₐE₅Eᵢᵢᵢ | AlkLEᵥEᵥᵢ | E₄* | Eₐ |
| 372 | E_{1b}E₅Eᵢᵢᵢ | AlkLEᵥEᵥᵢ | E₄* | Eₐ |
| 373 | E₁ₐEᵢᵢᵢE₄ | AlkLEᵥEᵥᵢ | E₄* | Eₐ |
| 374 | E_{1b}EᵢᵢᵢE₄ | AlkLEᵥEᵥᵢ | E₄* | Eₐ |
| 375 | E₁ₐE₅EᵢᵢᵢE₄ | AlkLEᵥEᵥᵢ | E₄* | Eₐ |
| 376 | E_{1b}E₅EᵢᵢᵢE₄ | AlkLEᵥEᵥᵢ | E₄* | Eₐ |
| 377 | E₁ₐEᵢᵥ | AlkLEᵥEᵥᵢ | E₄* | Eₐ |
| 378 | E_{1b}Eᵢᵥ | AlkLEᵥEᵥᵢ | E₄* | Eₐ |
| 379 | E₁ₐE₅Eᵢᵥ | AlkLEᵥEᵥᵢ | E₄* | Eₐ |
| 380 | E_{1b}E₅Eᵢᵥ | AlkLEᵥEᵥᵢ | E₄* | Eₐ |
| 381 | E₁ₐEᵢᵥE₄ | AlkLEᵥEᵥᵢ | E₄* | Eₐ |
| 382 | E_{1b}EᵢᵥE₄ | AlkLEᵥEᵥᵢ | E₄* | Eₐ |
| 383 | E₁ₐE₅EᵢᵥE₄ | AlkLEᵥEᵥᵢ | E₄* | Eₐ |
| 384 | E_{1b}E₅EᵢᵥE₄ | AlkLEᵥEᵥᵢ | E₄* | Eₐ |
| 385 | E₁ₐ | EᵥEᵥᵢ | | E_{b} |
| 386 | E_{1b} | EᵥEᵥᵢ | | E_{b} |
| 387 | E₁ₐE₅ | EᵥEᵥᵢ | | E_{b} |
| 388 | E_{1b}E₅ | EᵥEᵥᵢ | | E_{b} |
| 389 | E₁ₐEᵢ | EᵥEᵥᵢ | | E_{b} |
| 390 | E_{1b}Eᵢ | EᵥEᵥᵢ | | E_{b} |
| 391 | E₁ₐEᵢᵢ | EᵥEᵥᵢ | | E_{b} |
| 392 | E_{1b}Eᵢᵢ | EᵥEᵥᵢ | | E_{b} |
| 393 | E₁ₐEᵢᵢE_{iib} | EᵥEᵥᵢ | | E_{b} |
| 394 | E_{1b}EᵢᵢE_{iib} | EᵥEᵥᵢ | | E_{b} |
| 395 | E₁ₐE₅Eᵢ | EᵥEᵥᵢ | | E_{b} |
| 396 | E_{1b}E₅Eᵢ | EᵥEᵥᵢ | | E_{b} |
| 397 | E₁ₐE₅Eᵢᵢ | EᵥEᵥᵢ | | E_{b} |
| 398 | E_{1b}E₅Eᵢᵢ | EᵥEᵥᵢ | | E_{b} |
| 399 | E₁ₐE₅EᵢᵢE_{iib} | EᵥEᵥᵢ | | E_{b} |
| 400 | E_{1b}E₅EᵢᵢE_{iib} | EᵥEᵥᵢ | | E_{b} |
| 401 | E₁ₐE₄ | EᵥEᵥᵢ | | E_{b} |
| 402 | E_{1b}E₄ | EᵥEᵥᵢ | | E_{b} |
| 403 | E₁ₐE₅E₄ | EᵥEᵥᵢ | | E_{b} |
| 404 | E_{1b}E₅E₄ | EᵥEᵥᵢ | | E_{b} |
| 405 | E₁ₐEᵢE₄ | EᵥEᵥᵢ | | E_{b} |
| 406 | E_{1b}EᵢE₄ | EᵥEᵥᵢ | | E_{b} |
| 407 | E₁ₐEᵢᵢE₄ | EᵥEᵥᵢ | | E_{b} |
| 408 | E_{1b}EᵢᵢE₄ | EᵥEᵥᵢ | | E_{b} |
| 409 | E₁ₐEᵢᵢE_{iib}E₄ | EᵥEᵥᵢ | | E_{b} |
| 410 | E_{1b}EᵢᵢE_{iib}E₄ | EᵥEᵥᵢ | | E_{b} |
| 411 | E₁ₐE₅EᵢE₄ | EᵥEᵥᵢ | | E_{b} |
| 412 | E_{1b}E₅EᵢE₄ | EᵥEᵥᵢ | | E_{b} |
| 413 | E₁ₐE₅EᵢᵢE₄ | EᵥEᵥᵢ | | E_{b} |
| 414 | E_{1b}E₅EᵢᵢE₄ | EᵥEᵥᵢ | | E_{b} |
| 415 | E₁ₐE₅EᵢᵢE_{iib}E₄ | EᵥEᵥᵢ | | E_{b} |
| 416 | E_{1b}E₅EᵢᵢE_{iib}E₄ | EᵥEᵥᵢ | | E_{b} |
| 417 | E₁ₐEᵢᵢᵢ | EᵥEᵥᵢ | | E_{b} |
| 418 | E_{1b}Eᵢᵢᵢ | EᵥEᵥᵢ | | E_{b} |
| 419 | E₁ₐE₅Eᵢᵢᵢ | EᵥEᵥᵢ | | E_{b} |
| 420 | E_{1b}E₅Eᵢᵢᵢ | EᵥEᵥᵢ | | E_{b} |
| 421 | E₁ₐEᵢᵢᵢE₄ | EᵥEᵥᵢ | | E_{b} |
| 422 | E_{1b}EᵢᵢᵢE₄ | EᵥEᵥᵢ | | E_{b} |
| 423 | E₁ₐE₅EᵢᵢᵢE₄ | EᵥEᵥᵢ | | E_{b} |
| 424 | E_{1b}E₅EᵢᵢᵢE₄ | EᵥEᵥᵢ | | E_{b} |
| 425 | E₁ₐEᵢᵥ | EᵥEᵥᵢ | | E_{b} |
| 426 | E_{1b}Eᵢᵥ | EᵥEᵥᵢ | | E_{b} |
| 427 | E₁ₐE₅Eᵢᵥ | EᵥEᵥᵢ | | E_{b} |
| 428 | E_{1b}E₅Eᵢᵥ | EᵥEᵥᵢ | | E_{b} |
| 429 | E₁ₐEᵢᵥE₄ | EᵥEᵥᵢ | | E_{b} |
| 430 | E_{1b}EᵢᵥE₄ | EᵥEᵥᵢ | | E_{b} |
| 431 | E₁ₐE₅EᵢᵥE₄ | EᵥEᵥᵢ | | E_{b} |
| 432 | E_{1b}E₅EᵢᵥE₄ | EᵥEᵥᵢ | | E_{b} |
| 433 | E₁ₐ | EᵥEᵥᵢ | E₄* | E_{b} |
| 434 | E_{1b} | EᵥEᵥᵢ | E₄* | E_{b} |
| 435 | E₁ₐE₅ | EᵥEᵥᵢ | E₄* | E_{b} |
| 436 | E_{1b}E₅ | EᵥEᵥᵢ | E₄* | E_{b} |
| 437 | E₁ₐEᵢ | EᵥEᵥᵢ | E₄* | E_{b} |
| 438 | E_{1b}Eᵢ | EᵥEᵥᵢ | E₄* | E_{b} |
| 439 | E₁ₐEᵢᵢ | EᵥEᵥᵢ | E₄* | E_{b} |
| 440 | E_{1b}Eᵢᵢ | EᵥEᵥᵢ | E₄* | E_{b} |
| 441 | E₁ₐEᵢᵢE_{iib} | EᵥEᵥᵢ | E₄* | E_{b} |
| 442 | E_{1b}EᵢᵢE_{iib} | EᵥEᵥᵢ | E₄* | E_{b} |
| 443 | E₁ₐE₅Eᵢ | EᵥEᵥᵢ | E₄* | E_{b} |
| 444 | E_{1b}E₅Eᵢ | EᵥEᵥᵢ | E₄* | E_{b} |
| 445 | E₁ₐE₅Eᵢᵢ | EᵥEᵥᵢ | E₄* | E_{b} |
| 446 | E_{1b}E₅Eᵢᵢ | EᵥEᵥᵢ | E₄* | E_{b} |
| 447 | E₁ₐE₅EᵢᵢE_{iib} | EᵥEᵥᵢ | E₄* | E_{b} |
| 448 | E_{1b}E₅EᵢᵢE_{iib} | EᵥEᵥᵢ | E₄* | E_{b} |
| 449 | E₁ₐE₄ | EᵥEᵥᵢ | E₄* | E_{b} |
| 450 | E_{1b}E₄ | EᵥEᵥᵢ | E₄* | E_{b} |
| 451 | E₁ₐE₅E₄ | EᵥEᵥᵢ | E₄* | E_{b} |
| 452 | E_{1b}E₅E₄ | EᵥEᵥᵢ | E₄* | E_{b} |
| 453 | E₁ₐEᵢE₄ | EᵥEᵥᵢ | E₄* | E_{b} |
| 454 | E_{1b}EᵢE₄ | EᵥEᵥᵢ | E₄* | E_{b} |
| 455 | E₁ₐEᵢᵢE₄ | EᵥEᵥᵢ | E₄* | E_{b} |
| 456 | E_{1b}EᵢᵢE₄ | EᵥEᵥᵢ | E₄* | E_{b} |
| 457 | E₁ₐEᵢᵢE_{iib}E₄ | EᵥEᵥᵢ | E₄* | E_{b} |
| 458 | E_{1b}EᵢᵢE_{iib}E₄ | EᵥEᵥᵢ | E₄* | E_{b} |
| 459 | E₁ₐE₅EᵢE₄ | EᵥEᵥᵢ | E₄* | E_{b} |
| 460 | E_{1b}E₅EᵢE₄ | EᵥEᵥᵢ | E₄* | E_{b} |
| 461 | E₁ₐE₅EᵢᵢE₄ | EᵥEᵥᵢ | E₄* | E_{b} |
| 462 | E_{1b}E₅EᵢᵢE₄ | EᵥEᵥᵢ | E₄* | E_{b} |
| 463 | E₁ₐE₅EᵢᵢE_{iib}E₄ | EᵥEᵥᵢ | E₄* | E_{b} |
| 464 | E_{1b}E₅EᵢᵢE_{iib}E₄ | EᵥEᵥᵢ | E₄* | E_{b} |
| 465 | E₁ₐEᵢᵢᵢ | EᵥEᵥᵢ | E₄* | E_{b} |
| 466 | E_{1b}Eᵢᵢᵢ | EᵥEᵥᵢ | E₄* | E_{b} |
| 467 | E₁ₐE₅Eᵢᵢᵢ | EᵥEᵥᵢ | E₄* | E_{b} |
| 468 | E_{1b}E₅Eᵢᵢᵢ | EᵥEᵥᵢ | E₄* | E_{b} |
| 469 | E₁ₐEᵢᵢᵢE₄ | EᵥEᵥᵢ | E₄* | E_{b} |
| 470 | E_{1b}EᵢᵢᵢE₄ | EᵥEᵥᵢ | E₄* | E_{b} |
| 471 | E₁ₐE₅EᵢᵢᵢE₄ | EᵥEᵥᵢ | E₄* | E_{b} |
| 472 | E_{1b}E₅EᵢᵢᵢE₄ | EᵥEᵥᵢ | E₄* | E_{b} |
| 473 | E₁ₐEᵢᵥ | EᵥEᵥᵢ | E₄* | E_{b} |
| 474 | E_{1b}Eᵢᵥ | EᵥEᵥᵢ | E₄* | E_{b} |
| 475 | E₁ₐE₅Eᵢᵥ | EᵥEᵥᵢ | E₄* | E_{b} |
| 476 | E_{1b}E₅Eᵢᵥ | EᵥEᵥᵢ | E₄* | E_{b} |
| 477 | E₁ₐEᵢᵥE₄ | EᵥEᵥᵢ | E₄* | E_{b} |
| 478 | E_{1b}EᵢᵥE₄ | EᵥEᵥᵢ | E₄* | E_{b} |
| 479 | E₁ₐE₅EᵢᵥE₄ | EᵥEᵥᵢ | E₄* | E_{b} |
| 480 | E_{1b}E₅EᵢᵥE₄ | EᵥEᵥᵢ | E₄* | E_{b} |
| 481 | E₁ₐ | AlkLEᵥEᵥᵢ | | E_{b} |
| 482 | E_{1b} | AlkLEᵥEᵥᵢ | | E_{b} |
| 483 | E₁ₐE₅ | AlkLEᵥEᵥᵢ | | E_{b} |
| 484 | E_{1b}E₅ | AlkLEᵥEᵥᵢ | | E_{b} |
| 485 | E₁ₐEᵢ | AlkLEᵥEᵥᵢ | | E_{b} |
| 486 | E_{1b}Eᵢ | AlkLEᵥEᵥᵢ | | E_{b} |
| 487 | E₁ₐEᵢᵢ | AlkLEᵥEᵥᵢ | | E_{b} |
| 488 | E_{1b}Eᵢᵢ | AlkLEᵥEᵥᵢ | | E_{b} |
| 489 | E₁ₐEᵢᵢE_{iib} | AlkLEᵥEᵥᵢ | | E_{b} |
| 490 | E_{1b}EᵢᵢE_{iib} | AlkLEᵥEᵥᵢ | | E_{b} |
| 491 | E₁ₐE₅Eᵢ | AlkLEᵥEᵥᵢ | | E_{b} |
| 492 | E_{1b}E₅Eᵢ | AlkLEᵥEᵥᵢ | | E_{b} |
| 493 | E₁ₐE₅Eᵢᵢ | AlkLEᵥEᵥᵢ | | E_{b} |
| 494 | E_{1b}E₅Eᵢᵢ | AlkLEᵥEᵥᵢ | | E_{b} |
| 495 | E₁ₐE₅EᵢᵢE_{iib} | AlkLEᵥEᵥᵢ | | E_{b} |
| 496 | E_{1b}E₅EᵢᵢE_{iib} | AlkLEᵥEᵥᵢ | | E_{b} |
| 497 | E₁ₐE₄ | AlkLEᵥEᵥᵢ | | E_{b} |
| 498 | E_{1b}E₄ | AlkLEᵥEᵥᵢ | | E_{b} |
| 499 | E₁ₐE₅E₄ | AlkLEᵥEᵥᵢ | | E_{b} |
| 500 | E_{1b}E₅E₄ | AlkLEᵥEᵥᵢ | | E_{b} |
| 501 | E₁ₐEᵢE₄ | AlkLEᵥEᵥᵢ | | E_{b} |
| 502 | E_{1b}EᵢE₄ | AlkLEᵥEᵥᵢ | | E_{b} |
| 503 | E₁ₐEᵢᵢE₄ | AlkLEᵥEᵥᵢ | | E_{b} |
| 504 | E_{1b}EᵢᵢE₄ | AlkLEᵥEᵥᵢ | | E_{b} |
| 505 | E₁ₐEᵢᵢE_{iib}E₄ | AlkLEᵥEᵥᵢ | | E_{b} |
| 506 | E_{1b}EᵢᵢE_{iib}E₄ | AlkLEᵥEᵥᵢ | | E_{b} |
| 507 | E₁ₐE₅EᵢE₄ | AlkLEᵥEᵥᵢ | | E_{b} |
| 508 | E_{1b}E₅EᵢE₄ | AlkLEᵥEᵥᵢ | | E_{b} |
| 509 | E₁ₐE₅EᵢᵢE₄ | AlkLEᵥEᵥᵢ | | E_{b} |
| 510 | E_{1b}E₅EᵢᵢE₄ | AlkLEᵥEᵥᵢ | | E_{b} |
| 511 | E₁ₐE₅EᵢᵢE_{iib}E₄ | AlkLEᵥEᵥᵢ | | E_{b} |
| 512 | E_{1b}E₅EᵢᵢE_{iib}E₄ | AlkLEᵥEᵥᵢ | | E_{b} |
| 513 | E₁ₐEᵢᵢᵢ | AlkLEᵥEᵥᵢ | | E_{b} |
| 514 | E_{1b}Eᵢᵢᵢ | AlkLEᵥEᵥᵢ | | E_{b} |
| 515 | E₁ₐE₅Eᵢᵢᵢ | AlkLEᵥEᵥᵢ | | E_{b} |
| 516 | E_{1b}E₅Eᵢᵢᵢ | AlkLEᵥEᵥᵢ | | E_{b} |
| 517 | E₁ₐEᵢᵢᵢE₄ | AlkLEᵥEᵥᵢ | | E_{b} |
| 518 | E_{1b}EᵢᵢᵢE₄ | AlkLEᵥEᵥᵢ | | E_{b} |
| 519 | E₁ₐE₅EᵢᵢᵢE₄ | AlkLEᵥEᵥᵢ | | E_{b} |
| 520 | E_{1b}E₅EᵢᵢᵢE₄ | AlkLEᵥEᵥᵢ | | E_{b} |
| 521 | E₁ₐEᵢᵥ | AlkLEᵥEᵥᵢ | | E_{b} |
| 522 | E_{1b}Eᵢᵥ | AlkLEᵥEᵥᵢ | | E_{b} |
| 523 | E₁ₐE₅Eᵢᵥ | AlkLEᵥEᵥᵢ | | E_{b} |
| 524 | E_{1b}E₅Eᵢᵥ | AlkLEᵥEᵥᵢ | | E_{b} |
| 525 | E₁ₐEᵢᵥE₄ | AlkLEᵥEᵥᵢ | | E_{b} |
| 526 | E_{1b}EᵢᵥE₄ | AlkLEᵥEᵥᵢ | | E_{b} |
| 527 | E₁ₐE₅EᵢᵥE₄ | AlkLEᵥEᵥᵢ | | E_{b} |
| 528 | E_{1b}E₅EᵢᵥE₄ | AlkLEᵥEᵥᵢ | | E_{b} |
| 529 | E₁ₐ | AlkLEᵥEᵥᵢ | E₄* | E_{b} |
| 530 | E_{1b} | AlkLEᵥEᵥᵢ | E₄* | E_{b} |
| 531 | E₁ₐE₅ | AlkLEᵥEᵥᵢ | E₄* | E_{b} |
| 532 | E_{1b}E₅ | AlkLEᵥEᵥᵢ | E₄* | E_{b} |
| 533 | E₁ₐEᵢ | AlkLEᵥEᵥᵢ | E₄* | E_{b} |
| 534 | E_{1b}Eᵢ | AlkLEᵥEᵥᵢ | E₄* | E_{b} |
| 535 | E₁ₐEᵢᵢ | AlkLEᵥEᵥᵢ | E₄* | E_{b} |
| 536 | E_{1b}Eᵢᵢ | AlkLEᵥEᵥᵢ | E₄* | E_{b} |
| 537 | E₁ₐEᵢᵢE_{iib} | AlkLEᵥEᵥᵢ | E₄* | E_{b} |
| 538 | E_{1b}EᵢᵢE_{iib} | AlkLEᵥEᵥᵢ | E₄* | E_{b} |
| 539 | E₁ₐE₅Eᵢ | AlkLEᵥEᵥᵢ | E₄* | E_{b} |
| 540 | E_{1b}E₅Eᵢ | AlkLEᵥEᵥᵢ | E₄* | E_{b} |
| 541 | E₁ₐE₅Eᵢᵢ | AlkLEᵥEᵥᵢ | E₄* | E_{b} |
| 542 | E_{1b}E₅Eᵢᵢ | AlkLEᵥEᵥᵢ | E₄* | E_{b} |
| 543 | E₁ₐE₅EᵢᵢE_{iib} | AlkLEᵥEᵥᵢ | E₄* | E_{b} |
| 544 | E_{1b}E₅EᵢᵢE_{iib} | AlkLEᵥEᵥᵢ | E₄* | E_{b} |
| 545 | E₁ₐE₄ | AlkLEᵥEᵥᵢ | E₄* | E_{b} |
| 546 | E_{1b}E₄ | AlkLEᵥEᵥᵢ | E₄* | E_{b} |
| 547 | E₁ₐE₅E₄ | AlkLEᵥEᵥᵢ | E₄* | E_{b} |
| 548 | E_{1b}E₅E₄ | AlkLEᵥEᵥᵢ | E₄* | E_{b} |
| 549 | E₁ₐEᵢE₄ | AlkLEᵥEᵥᵢ | E₄* | E_{b} |
| 550 | E_{1b}EᵢE₄ | AlkLEᵥEᵥᵢ | E₄* | E_{b} |
| 551 | E₁ₐEᵢᵢE₄ | AlkLEᵥEᵥᵢ | E₄* | E_{b} |
| 552 | E_{1b}EᵢᵢE₄ | AlkLEᵥEᵥᵢ | E₄* | E_{b} |
| 553 | E₁ₐEᵢᵢE_{iib}E₄ | AlkLEᵥEᵥᵢ | E₄* | E_{b} |
| 554 | E_{1b}EᵢᵢE_{iib}E₄ | AlkLEᵥEᵥᵢ | E₄* | E_{b} |
| 555 | E₁ₐE₅EᵢE₄ | AlkLEᵥEᵥᵢ | E₄* | E_{b} |
| 556 | E_{1b}E₅EᵢE₄ | AlkLEᵥEᵥᵢ | E₄* | E_{b} |
| 557 | E₁ₐE₅EᵢᵢE₄ | AlkLEᵥEᵥᵢ | E₄* | E_{b} |
| 558 | E_{1b}E₅EᵢᵢE₄ | AlkLEᵥEᵥᵢ | E₄* | E_{b} |
| 559 | E₁ₐE₅EᵢᵢE_{iib}E₄ | AlkLEᵥEᵥᵢ | E₄* | E_{b} |
| 560 | E_{1b}E₅EᵢᵢE_{iib}E₄ | AlkLEᵥEᵥᵢ | E₄* | E_{b} |
| 561 | E₁ₐEᵢᵢᵢ | AlkLEᵥEᵥᵢ | E₄* | E_{b} |
| 562 | E_{1b}Eᵢᵢᵢ | AlkLEᵥEᵥᵢ | E₄* | E_{b} |
| 563 | E₁ₐE₅Eᵢᵢᵢ | AlkLEᵥEᵥᵢ | E₄* | E_{b} |
| 564 | E_{1b}E₅Eᵢᵢᵢ | AlkLEᵥEᵥᵢ | E₄* | E_{b} |
| 565 | E₁ₐEᵢᵢᵢE₄ | AlkLEᵥEᵥᵢ | E₄* | E_{b} |
| 566 | E_{1b}EᵢᵢᵢE₄ | AlkLEᵥEᵥᵢ | E₄* | E_{b} |
| 567 | E₁ₐE₅EᵢᵢᵢE₄ | AlkLEᵥEᵥᵢ | E₄* | E_{b} |
| 568 | E_{1b}E₅EᵢᵢᵢE₄ | AlkLEᵥEᵥᵢ | E₄* | E_{b} |
| 569 | E₁ₐEᵢᵥ | AlkLEᵥEᵥᵢ | E₄* | E_{b} |
| 570 | E_{1b}Eᵢᵥ | AlkLEᵥEᵥᵢ | E₄* | E_{b} |
| 571 | E₁ₐE₅Eᵢᵥ | AlkLEᵥEᵥᵢ | E₄* | E_{b} |
| 572 | E_{1b}E₅Eᵢᵥ | AlkLEᵥEᵥᵢ | E₄* | E_{b} |
| 573 | E₁ₐEᵢᵥE₄ | AlkLEᵥEᵥᵢ | E₄* | E_{b} |
| 574 | E_{1b}EᵢᵥE₄ | AlkLEᵥEᵥᵢ | E₄* | E_{b} |
| 575 | E₁ₐE₅EᵢᵥE₄ | AlkLEᵥEᵥᵢ | E₄* | E_{b} |
| 576 | E_{1b}E₅EᵢᵥE₄ | AlkLEᵥEᵥᵢ | E₄* | E_{b} |
| 577 | E₁ₐ | EᵥEᵥᵢ | | E_{d} |
| 578 | E_{1b} | EᵥEᵥᵢ | | E_{d} |
| 579 | E₁ₐE₅ | EᵥEᵥᵢ | | E_{d} |
| 580 | E_{1b}E₅ | EᵥEᵥᵢ | | E_{d} |
| 581 | E₁ₐEᵢ | EᵥEᵥᵢ | | E_{d} |
| 582 | E_{1b}Eᵢ | EᵥEᵥᵢ | | E_{d} |
| 583 | E₁ₐEᵢᵢ | EᵥEᵥᵢ | | E_{d} |
| 584 | E_{1b}Eᵢᵢ | EᵥEᵥᵢ | | E_{d} |
| 585 | E₁ₐEᵢᵢE_{iib} | EᵥEᵥᵢ | | E_{d} |
| 586 | E_{1b}EᵢᵢE_{iib} | EᵥEᵥᵢ | | E_{d} |
| 587 | E₁ₐE₅Eᵢ | EᵥEᵥᵢ | | E_{d} |
| 588 | E_{1b}E₅Eᵢ | EᵥEᵥᵢ | | E_{d} |
| 589 | E₁ₐE₅Eᵢᵢ | EᵥEᵥᵢ | | E_{d} |
| 590 | E_{1b}E₅Eᵢᵢ | EᵥEᵥᵢ | | E_{d} |
| 591 | E₁ₐE₅EᵢᵢE_{iib} | EᵥEᵥᵢ | | E_{d} |
| 592 | E_{1b}E₅EᵢᵢE_{iib} | EᵥEᵥᵢ | | E_{d} |
| 593 | E₁ₐE₄ | EᵥEᵥᵢ | | E_{d} |
| 594 | E_{1b}E₄ | EᵥEᵥᵢ | | E_{d} |
| 595 | E₁ₐE₅E₄ | EᵥEᵥᵢ | | E_{d} |
| 596 | E_{1b}E₅E₄ | EᵥEᵥᵢ | | E_{d} |
| 597 | E₁ₐEᵢE₄ | EᵥEᵥᵢ | | E_{d} |
| 598 | E_{1b}EᵢE₄ | EᵥEᵥᵢ | | E_{d} |
| 599 | E₁ₐEᵢᵢE₄ | EᵥEᵥᵢ | | E_{d} |
| 600 | E_{1b}EᵢᵢE₄ | EᵥEᵥᵢ | | E_{d} |
| 601 | E₁ₐEᵢᵢE_{iib}E₄ | EᵥEᵥᵢ | | E_{d} |
| 602 | E_{1b}EᵢᵢE_{iib}E₄ | EᵥEᵥᵢ | | E_{d} |
| 603 | E₁ₐE₅EᵢE₄ | EᵥEᵥᵢ | | E_{d} |
| 604 | E_{1b}E₅EᵢE₄ | EᵥEᵥᵢ | | E_{d} |
| 605 | E₁ₐE₅EᵢᵢE₄ | EᵥEᵥᵢ | | E_{d} |
| 606 | E_{1b}E₅EᵢᵢE₄ | EᵥEᵥᵢ | | E_{d} |
| 607 | E₁ₐE₅EᵢᵢE_{iib}E₄ | EᵥEᵥᵢ | | E_{d} |
| 608 | E_{1b}E₅EᵢᵢE_{iib}E₄ | EᵥEᵥᵢ | | E_{d} |
| 609 | E₁ₐEᵢᵢᵢ | EᵥEᵥᵢ | | E_{d} |
| 610 | E_{1b}Eᵢᵢᵢ | EᵥEᵥᵢ | | E_{d} |
| 611 | E₁ₐE₅Eᵢᵢᵢ | EᵥEᵥᵢ | | E_{d} |
| 612 | E_{1b}E₅Eᵢᵢᵢ | EᵥEᵥᵢ | | E_{d} |
| 613 | E₁ₐEᵢᵢᵢE₄ | EᵥEᵥᵢ | | E_{d} |
| 614 | E_{1b}EᵢᵢᵢE₄ | EᵥEᵥᵢ | | E_{d} |
| 615 | E₁ₐE₅EᵢᵢᵢE₄ | EᵥEᵥᵢ | | E_{d} |
| 616 | E_{1b}E₅EᵢᵢᵢE₄ | EᵥEᵥᵢ | | E_{d} |
| 617 | E₁ₐEᵢᵥ | EᵥEᵥᵢ | | E_{d} |
| 618 | E_{1b}Eᵢᵥ | EᵥEᵥᵢ | | E_{d} |
| 619 | E₁ₐE₅Eᵢᵥ | EᵥEᵥᵢ | | E_{d} |
| 620 | E_{1b}E₅Eᵢᵥ | EᵥEᵥᵢ | | E_{d} |
| 621 | E₁ₐEᵢᵥE₄ | EᵥEᵥᵢ | | E_{d} |
| 622 | E_{1b}EᵢᵥE₄ | EᵥEᵥᵢ | | E_{d} |
| 623 | E₁ₐE₅EᵢᵥE₄ | EᵥEᵥᵢ | | E_{d} |
| 624 | E_{1b}E₅EᵢᵥE₄ | EᵥEᵥᵢ | | E_{d} |
| 625 | E₁ₐ | EᵥEᵥᵢ | E₄* | E_{d} |
| 626 | E_{1b} | EᵥEᵥᵢ | E₄* | E_{d} |
| 627 | E₁ₐE₅ | EᵥEᵥᵢ | E₄* | E_{d} |
| 628 | E_{1b}E₅ | EᵥEᵥᵢ | E₄* | E_{d} |
| 629 | E₁ₐEᵢ | EᵥEᵥᵢ | E₄* | E_{d} |
| 630 | E_{1b}Eᵢ | EᵥEᵥᵢ | E₄* | E_{d} |
| 631 | E₁ₐEᵢᵢ | EᵥEᵥᵢ | E₄* | E_{d} |
| 632 | E_{1b}Eᵢᵢ | EᵥEᵥᵢ | E₄* | E_{d} |
| 633 | E₁ₐEᵢᵢE_{iib} | EᵥEᵥᵢ | E₄* | E_{d} |
| 634 | E_{1b}EᵢᵢE_{iib} | EᵥEᵥᵢ | E₄* | E_{d} |
| 635 | E₁ₐE₅Eᵢ | EᵥEᵥᵢ | E₄* | E_{d} |
| 636 | E_{1b}E₅Eᵢ | EᵥEᵥᵢ | E₄* | E_{d} |
| 637 | E₁ₐE₅Eᵢᵢ | EᵥEᵥᵢ | E₄* | E_{d} |
| 638 | E_{1b}E₅Eᵢᵢ | EᵥEᵥᵢ | E₄* | E_{d} |
| 639 | E₁ₐE₅EᵢᵢE_{iib} | EᵥEᵥᵢ | E₄* | E_{d} |
| 640 | E_{1b}E₅EᵢᵢE_{iib} | EᵥEᵥᵢ | E₄* | E_{d} |
| 641 | E₁ₐE₄ | EᵥEᵥᵢ | E₄* | E_{d} |
| 642 | E_{1b}E₄ | EᵥEᵥᵢ | E₄* | E_{d} |
| 643 | E₁ₐE₅E₄ | EᵥEᵥᵢ | E₄* | E_{d} |
| 644 | E_{1b}E₅E₄ | EᵥEᵥᵢ | E₄* | E_{d} |
| 645 | E₁ₐEᵢE₄ | EᵥEᵥᵢ | E₄* | E_{d} |
| 646 | E_{1b}EᵢE₄ | EᵥEᵥᵢ | E₄* | E_{d} |
| 647 | E₁ₐEᵢᵢE₄ | EᵥEᵥᵢ | E₄* | E_{d} |
| 648 | E_{1b}EᵢᵢE₄ | EᵥEᵥᵢ | E₄* | E_{d} |
| 649 | E₁ₐEᵢᵢE_{iib}E₄ | EᵥEᵥᵢ | E₄* | E_{d} |
| 650 | E_{1b}EᵢᵢE_{iib}E₄ | EᵥEᵥᵢ | E₄* | E_{d} |
| 651 | E₁ₐE₅EᵢE₄ | EᵥEᵥᵢ | E₄* | E_{d} |
| 652 | E_{1b}E₅EᵢE₄ | EᵥEᵥᵢ | E₄* | E_{d} |
| 653 | E₁ₐE₅EᵢᵢE₄ | EᵥEᵥᵢ | E₄* | E_{d} |
| 654 | E_{1b}E₅EᵢᵢE₄ | EᵥEᵥᵢ | E₄* | E_{d} |
| 655 | E₁ₐE₅EᵢᵢE_{iib}E₄ | EᵥEᵥᵢ | E₄* | E_{d} |
| 656 | E_{1b}E₅EᵢᵢE_{iib}E₄ | EᵥEᵥᵢ | E₄* | E_{d} |
| 657 | E₁ₐEᵢᵢᵢ | EᵥEᵥᵢ | E₄* | E_{d} |
| 658 | E_{1b}Eᵢᵢᵢ | EᵥEᵥᵢ | E₄* | E_{d} |
| 659 | E₁ₐE₅Eᵢᵢᵢ | EᵥEᵥᵢ | E₄* | E_{d} |
| 660 | E_{1b}E₅Eᵢᵢᵢ | EᵥEᵥᵢ | E₄* | E_{d} |
| 661 | E₁ₐEᵢᵢᵢE₄ | EᵥEᵥᵢ | E₄* | E_{d} |
| 662 | E_{1b}EᵢᵢᵢE₄ | EᵥEᵥᵢ | E₄* | E_{d} |
| 663 | E₁ₐE₅EᵢᵢᵢE₄ | EᵥEᵥᵢ | E₄* | E_{d} |
| 664 | E_{1b}E₅EᵢᵢᵢE₄ | EᵥEᵥᵢ | E₄* | E_{d} |
| 665 | E₁ₐEᵢᵥ | EᵥEᵥᵢ | E₄* | E_{d} |
| 666 | E_{1b}Eᵢᵥ | EᵥEᵥᵢ | E₄* | E_{d} |
| 667 | E₁ₐE₅Eᵢᵥ | EᵥEᵥᵢ | E₄* | E_{d} |
| 668 | E_{1b}E₅Eᵢᵥ | EᵥEᵥᵢ | E₄* | E_{d} |
| 669 | E₁ₐEᵢᵥE₄ | EᵥEᵥᵢ | E₄* | E_{d} |
| 670 | E_{1b}EᵢᵥE₄ | EᵥEᵥᵢ | E₄* | E_{d} |
| 671 | E₁ₐE₅EᵢᵥE₄ | EᵥEᵥᵢ | E₄* | E_{d} |
| 672 | E_{1b}E₅EᵢᵥE₄ | EᵥEᵥᵢ | E₄* | E_{d} |
| 673 | E₁ₐ | AlkLEᵥEᵥᵢ | | E_{d} |
| 674 | E_{1b} | AlkLEᵥEᵥᵢ | | E_{d} |
| 675 | E₁ₐE₅ | AlkLEᵥEᵥᵢ | | E_{d} |
| 676 | E_{1b}E₅ | AlkLEᵥEᵥᵢ | | E_{d} |
| 677 | E₁ₐEᵢ | AlkLEᵥEᵥᵢ | | E_{d} |
| 678 | E_{1b}Eᵢ | AlkLEᵥEᵥᵢ | | E_{d} |
| 679 | E₁ₐEᵢᵢ | AlkLEᵥEᵥᵢ | | E_{d} |
| 680 | E_{1b}Eᵢᵢ | AlkLEᵥEᵥᵢ | | E_{d} |
| 681 | E₁ₐEᵢᵢE_{iib} | AlkLEᵥEᵥᵢ | | E_{d} |
| 682 | E_{1b}EᵢᵢE_{iib} | AlkLEᵥEᵥᵢ | | E_{d} |
| 683 | E₁ₐE₅Eᵢ | AlkLEᵥEᵥᵢ | | E_{d} |
| 684 | E_{1b}E₅Eᵢ | AlkLEᵥEᵥᵢ | | E_{d} |
| 685 | E₁ₐE₅Eᵢᵢ | AlkLEᵥEᵥᵢ | | E_{d} |
| 686 | E_{1b}E₅Eᵢᵢ | AlkLEᵥEᵥᵢ | | E_{d} |
| 687 | E₁ₐE₅EᵢᵢE_{iib} | AlkLEᵥEᵥᵢ | | E_{d} |
| 688 | E_{1b}E₅EᵢᵢE_{iib} | AlkLEᵥEᵥᵢ | | E_{d} |
| 689 | E₁ₐE₄ | AlkLEᵥEᵥᵢ | | E_{d} |
| 690 | E_{1b}E₄ | AlkLEᵥEᵥᵢ | | E_{d} |
| 691 | E₁ₐE₅E₄ | AlkLEᵥEᵥᵢ | | E_{d} |
| 692 | E_{1b}E₅E₄ | AlkLEᵥEᵥᵢ | | E_{d} |
| 693 | E₁ₐEᵢE₄ | AlkLEᵥEᵥᵢ | | E_{d} |
| 694 | E_{1b}EᵢE₄ | AlkLEᵥEᵥᵢ | | E_{d} |
| 695 | E₁ₐEᵢᵢE₄ | AlkLEᵥEᵥᵢ | | E_{d} |
| 696 | E_{1b}EᵢᵢE₄ | AlkLEᵥEᵥᵢ | | E_{d} |
| 697 | E₁ₐEᵢᵢE_{iib}E₄ | AlkLEᵥEᵥᵢ | | E_{d} |
| 698 | E_{1b}EᵢᵢE_{iib}E₄ | AlkLEᵥEᵥᵢ | | E_{d} |
| 699 | E₁ₐE₅EᵢE₄ | AlkLEᵥEᵥᵢ | | E_{d} |
| 700 | E_{1b}E₅EᵢE₄ | AlkLEᵥEᵥᵢ | | E_{d} |
| 701 | E₁ₐE₅EᵢᵢE₄ | AlkLEᵥEᵥᵢ | | E_{d} |
| 702 | E_{1b}E₅EᵢᵢE₄ | AlkLEᵥEᵥᵢ | | E_{d} |
| 703 | E₁ₐE₅EᵢᵢE_{iib}E₄ | AlkLEᵥEᵥᵢ | | E_{d} |
| 704 | E_{1b}E₅EᵢᵢE_{iib}E₄ | AlkLEᵥEᵥᵢ | | E_{d} |
| 705 | E₁ₐEᵢᵢᵢ | AlkLEᵥEᵥᵢ | | E_{d} |
| 706 | E_{1b}Eᵢᵢᵢ | AlkLEᵥEᵥᵢ | | E_{d} |
| 707 | E₁ₐE₅Eᵢᵢᵢ | AlkLEᵥEᵥᵢ | | E_{d} |
| 708 | E_{1b}E₅Eᵢᵢᵢ | AlkLEᵥEᵥᵢ | | E_{d} |
| 709 | E₁ₐEᵢᵢᵢE₄ | AlkLEᵥEᵥᵢ | | E_{d} |
| 710 | E_{1b}EᵢᵢᵢE₄ | AlkLEᵥEᵥᵢ | | E_{d} |
| 711 | E₁ₐE₅EᵢᵢᵢE₄ | AlkLEᵥEᵥᵢ | | E_{d} |
| 712 | E_{1b}E₅EᵢᵢᵢE₄ | AlkLEᵥEᵥᵢ | | E_{d} |
| 713 | E₁ₐEᵢᵥ | AlkLEᵥEᵥᵢ | | E_{d} |
| 714 | E_{1b}Eᵢᵥ | AlkLEᵥEᵥᵢ | | E_{d} |
| 715 | E₁ₐE₅Eᵢᵥ | AlkLEᵥEᵥᵢ | | E_{d} |
| 716 | E_{1b}E₅Eᵢᵥ | AlkLEᵥEᵥᵢ | | E_{d} |
| 717 | E₁ₐEᵢᵥE₄ | AlkLEᵥEᵥᵢ | | E_{d} |
| 718 | E_{1b}EᵢᵥE₄ | AlkLEᵥEᵥᵢ | | E_{d} |
| 719 | E₁ₐE₅EᵢᵥE₄ | AlkLEᵥEᵥᵢ | | E_{d} |
| 720 | E_{1b}E₅EᵢᵥE₄ | AlkLEᵥEᵥᵢ | | E_{d} |
| 721 | E₁ₐ | AlkLEᵥEᵥᵢ | E₄* | E_{d} |
| 722 | E_{1b} | AlkLEᵥEᵥᵢ | E₄* | E_{d} |
| 723 | E₁ₐE₅ | AlkLEᵥEᵥᵢ | E₄* | E_{d} |
| 724 | E_{1b}E₅ | AlkLEᵥEᵥᵢ | E₄* | E_{d} |
| 725 | E₁ₐEᵢ | AlkLEᵥEᵥᵢ | E₄* | E_{d} |
| 726 | E_{1b}Eᵢ | AlkLEᵥEᵥᵢ | E₄* | E_{d} |
| 727 | E₁ₐEᵢᵢ | AlkLEᵥEᵥᵢ | E₄* | E_{d} |
| 728 | E_{1b}Eᵢᵢ | AlkLEᵥEᵥᵢ | E₄* | E_{d} |
| 729 | E₁ₐEᵢᵢE_{iib} | AlkLEᵥEᵥᵢ | E₄* | E_{d} |
| 730 | E_{1b}EᵢᵢE_{iib} | AlkLEᵥEᵥᵢ | E₄* | E_{d} |
| 731 | E₁ₐE₅Eᵢ | AlkLEᵥEᵥᵢ | E₄* | E_{d} |
| 732 | E_{1b}E₅Eᵢ | AlkLEᵥEᵥᵢ | E₄* | E_{d} |
| 733 | E₁ₐE₅Eᵢᵢ | AlkLEᵥEᵥᵢ | E₄* | E_{d} |
| 734 | E_{1b}E₅Eᵢᵢ | AlkLEᵥEᵥᵢ | E₄* | E_{d} |
| 735 | E₁ₐE₅EᵢᵢE_{iib} | AlkLEᵥEᵥᵢ | E₄* | E_{d} |
| 736 | E_{1b}E₅EᵢᵢE_{iib} | AlkLEᵥEᵥᵢ | E₄* | E_{d} |
| 737 | E₁ₐE₄ | AlkLEᵥEᵥᵢ | E₄* | E_{d} |
| 738 | E_{1b}E₄ | AlkLEᵥEᵥᵢ | E₄* | E_{d} |
| 739 | E₁ₐE₅E₄ | AlkLEᵥEᵥᵢ | E₄* | E_{d} |
| 740 | E_{1b}E₅E₄ | AlkLEᵥEᵥᵢ | E₄* | E_{d} |
| 741 | E₁ₐEᵢE₄ | AlkLEᵥEᵥᵢ | E₄* | E_{d} |
| 742 | E_{1b}EᵢE₄ | AlkLEᵥEᵥᵢ | E₄* | E_{d} |
| 743 | E₁ₐEᵢᵢE₄ | AlkLEᵥEᵥᵢ | E₄* | E_{d} |
| 744 | E_{1b}EᵢᵢE₄ | AlkLEᵥEᵥᵢ | E₄* | E_{d} |
| 745 | E₁ₐEᵢᵢE_{iib}E₄ | AlkLEᵥEᵥᵢ | E₄* | E_{d} |
| 746 | E_{1b}EᵢᵢE_{iib}E₄ | AlkLEᵥEᵥᵢ | E₄* | E_{d} |
| 747 | E₁ₐE₅EᵢE₄ | AlkLEᵥEᵥᵢ | E₄* | E_{d} |
| 748 | E_{1b}E₅EᵢE₄ | AlkLEᵥEᵥᵢ | E₄* | E_{d} |
| 749 | E₁ₐE₅EᵢᵢE₄ | AlkLEᵥEᵥᵢ | E₄* | E_{d} |
| 750 | E_{1b}E₅EᵢᵢE₄ | AlkLEᵥEᵥᵢ | E₄* | E_{d} |
| 751 | E₁ₐE₅EᵢᵢE_{iib}E₄ | AlkLEᵥEᵥᵢ | E₄* | E_{d} |
| 752 | E_{1b}E₅EᵢᵢE_{iib}E₄ | AlkLEᵥEᵥᵢ | E₄* | E_{d} |
| 753 | E₁ₐEᵢᵢᵢ | AlkLEᵥEᵥᵢ | E₄* | E_{d} |
| 754 | E_{1b}Eᵢᵢᵢ | AlkLEᵥEᵥᵢ | E₄* | E_{d} |
| 755 | E₁ₐE₅Eᵢᵢᵢ | AlkLEᵥEᵥᵢ | E₄* | E_{d} |
| 756 | E_{1b}E₅Eᵢᵢᵢ | AlkLEᵥEᵥᵢ | E₄* | E_{d} |
| 757 | E₁ₐEᵢᵢᵢE₄ | AlkLEᵥEᵥᵢ | E₄* | E_{d} |
| 758 | E_{1b}EᵢᵢᵢE₄ | AlkLEᵥEᵥᵢ | E₄* | E_{d} |
| 759 | E₁ₐE₅EᵢᵢᵢE₄ | AlkLEᵥEᵥᵢ | E₄* | E_{d} |
| 760 | E_{1b}E₅EᵢᵢᵢE₄ | AlkLEᵥEᵥᵢ | E₄* | E_{d} |
| 761 | E₁ₐEᵢᵥ | AlkLEᵥEᵥᵢ | E₄* | E_{d} |
| 762 | E_{1b}Eᵢᵥ | AlkLEᵥEᵥᵢ | E₄* | E_{d} |
| 763 | E₁ₐE₅Eᵢᵥ | AlkLEᵥEᵥᵢ | E₄* | E_{d} |
| 764 | E_{1b}E₅Eᵢᵥ | AlkLEᵥEᵥᵢ | E₄* | E_{d} |
| 765 | E₁ₐEᵢᵥE₄ | AlkLEᵥEᵥᵢ | E₄* | E_{d} |
| 766 | E_{1b}EᵢᵥE₄ | AlkLEᵥEᵥᵢ | E₄* | E_{d} |
| 767 | E₁ₐE₅EᵢᵥE₄ | AlkLEᵥEᵥᵢ | E₄* | E_{d} |
| 768 | E_{1b}E₅EᵢᵥE₄ | AlkLEᵥEᵥᵢ | E₄* | E_{d} |
| 769 | E₁ₐ | EᵥEᵥᵢ | | Eₑ |
| 770 | E_{1b} | EᵥEᵥᵢ | | Eₑ |
| 771 | E₁ₐE₅ | EᵥEᵥᵢ | | Eₑ |
| 772 | E_{1b}E₅ | EᵥEᵥᵢ | | Eₑ |
| 773 | E₁ₐEᵢ | EᵥEᵥᵢ | | Eₑ |
| 774 | E_{1b}Eᵢ | EᵥEᵥᵢ | | Eₑ |
| 775 | E₁ₐEᵢᵢ | EᵥEᵥᵢ | | Eₑ |
| 776 | E_{1b}Eᵢᵢ | EᵥEᵥᵢ | | Eₑ |
| 777 | E₁ₐEᵢᵢE_{iib} | EᵥEᵥᵢ | | Eₑ |
| 778 | E_{1b}EᵢᵢE_{iib} | EᵥEᵥᵢ | | Eₑ |
| 779 | E₁ₐE₅Eᵢ | EᵥEᵥᵢ | | Eₑ |
| 780 | E_{1b}E₅Eᵢ | EᵥEᵥᵢ | | Eₑ |
| 781 | E₁ₐE₅Eᵢᵢ | EᵥEᵥᵢ | | Eₑ |
| 782 | E_{1b}E₅Eᵢᵢ | EᵥEᵥᵢ | | Eₑ |
| 783 | E₁ₐE₅EᵢᵢE_{iib} | EᵥEᵥᵢ | | Eₑ |
| 784 | E_{1b}E₅EᵢᵢE_{iib} | EᵥEᵥᵢ | | Eₑ |
| 785 | E₁ₐE₄ | EᵥEᵥᵢ | | Eₑ |
| 786 | E_{1b}E₄ | EᵥEᵥᵢ | | Eₑ |
| 787 | E₁ₐE₅E₄ | EᵥEᵥᵢ | | Eₑ |
| 788 | E_{1b}E₅E₄ | EᵥEᵥᵢ | | Eₑ |
| 789 | E₁ₐEᵢE₄ | EᵥEᵥᵢ | | Eₑ |
| 790 | E_{1b}EᵢE₄ | EᵥEᵥᵢ | | Eₑ |
| 791 | E₁ₐEᵢᵢE₄ | EᵥEᵥᵢ | | Eₑ |
| 792 | E_{1b}EᵢᵢE₄ | EᵥEᵥᵢ | | Eₑ |
| 793 | E₁ₐEᵢᵢE_{iib}E₄ | EᵥEᵥᵢ | | Eₑ |
| 794 | E_{1b}EᵢᵢE_{iib}E₄ | EᵥEᵥᵢ | | Eₑ |
| 795 | E₁ₐE₅EᵢE₄ | EᵥEᵥᵢ | | Eₑ |
| 796 | E_{1b}E₅EᵢE₄ | EᵥEᵥᵢ | | Eₑ |
| 797 | E₁ₐE₅EᵢᵢE₄ | EᵥEᵥᵢ | | Eₑ |
| 798 | E_{1b}E₅EᵢᵢE₄ | EᵥEᵥᵢ | | Eₑ |
| 799 | E₁ₐE₅EᵢᵢE_{iib}E₄ | EᵥEᵥᵢ | | Eₑ |
| 800 | E_{1b}E₅EᵢᵢE_{iib}E₄ | EᵥEᵥᵢ | | Eₑ |
| 801 | E₁ₐEᵢᵢᵢ | EᵥEᵥᵢ | | Eₑ |
| 802 | E_{1b}Eᵢᵢᵢ | EᵥEᵥᵢ | | Eₑ |
| 803 | E₁ₐE₅Eᵢᵢᵢ | EᵥEᵥᵢ | | Eₑ |
| 804 | E_{1b}E₅Eᵢᵢᵢ | EᵥEᵥᵢ | | Eₑ |
| 805 | E₁ₐEᵢᵢᵢE₄ | EᵥEᵥᵢ | | Eₑ |
| 806 | E_{1b}EᵢᵢᵢE₄ | EᵥEᵥᵢ | | Eₑ |
| 807 | E₁ₐE₅EᵢᵢᵢE₄ | EᵥEᵥᵢ | | Eₑ |
| 808 | E_{1b}E₅EᵢᵢᵢE₄ | EᵥEᵥᵢ | | Eₑ |
| 809 | E₁ₐEᵢᵥ | EᵥEᵥᵢ | | Eₑ |
| 810 | E_{1b}Eᵢᵥ | EᵥEᵥᵢ | | Eₑ |
| 811 | E₁ₐE₅Eᵢᵥ | EᵥEᵥᵢ | | Eₑ |
| 812 | E_{1b}E₅Eᵢᵥ | EᵥEᵥᵢ | | Eₑ |
| 813 | E₁ₐEᵢᵥE₄ | EᵥEᵥᵢ | | Eₑ |
| 814 | E_{1b}EᵢᵥE₄ | EᵥEᵥᵢ | | Eₑ |
| 815 | E₁ₐE₅EᵢᵥE₄ | EᵥEᵥᵢ | | Eₑ |
| 816 | E_{1b}E₅EᵢᵥE₄ | EᵥEᵥᵢ | | Eₑ |
| 817 | E₁ₐ | EᵥEᵥᵢ | E₄* | Eₑ |
| 818 | E_{1b} | EᵥEᵥᵢ | E₄* | Eₑ |
| 819 | E₁ₐE₅ | EᵥEᵥᵢ | E₄* | Eₑ |
| 820 | E_{1b}E₅ | EᵥEᵥᵢ | E₄* | Eₑ |
| 821 | E₁ₐEᵢ | EᵥEᵥᵢ | E₄* | Eₑ |
| 822 | E_{1b}Eᵢ | EᵥEᵥᵢ | E₄* | Eₑ |
| 823 | E₁ₐEᵢᵢ | EᵥEᵥᵢ | E₄* | Eₑ |
| 824 | E_{1b}Eᵢᵢ | EᵥEᵥᵢ | E₄* | Eₑ |
| 825 | E₁ₐEᵢᵢE_{iib} | EᵥEᵥᵢ | E₄* | Eₑ |
| 826 | E_{1b}EᵢᵢE_{iib} | EᵥEᵥᵢ | E₄* | Eₑ |
| 827 | E₁ₐE₅Eᵢ | EᵥEᵥᵢ | E₄* | Eₑ |
| 828 | E_{1b}E₅Eᵢ | EᵥEᵥᵢ | E₄* | Eₑ |
| 829 | E₁ₐE₅Eᵢᵢ | EᵥEᵥᵢ | E₄* | Eₑ |
| 830 | E_{1b}E₅Eᵢᵢ | EᵥEᵥᵢ | E₄* | Eₑ |
| 831 | E₁ₐE₅EᵢᵢE_{iib} | EᵥEᵥᵢ | E₄* | Eₑ |
| 832 | E_{1b}E₅EᵢᵢE_{iib} | EᵥEᵥᵢ | E₄* | Eₑ |
| 833 | E₁ₐE₄ | EᵥEᵥᵢ | E₄* | Eₑ |
| 834 | E_{1b}E₄ | EᵥEᵥᵢ | E₄* | Eₑ |
| 835 | E₁ₐE₅E₄ | EᵥEᵥᵢ | E₄* | Eₑ |
| 836 | E_{1b}E₅E₄ | EᵥEᵥᵢ | E₄* | Eₑ |
| 837 | E₁ₐEᵢE₄ | EᵥEᵥᵢ | E₄* | Eₑ |
| 838 | E_{1b}EᵢE₄ | EᵥEᵥᵢ | E₄* | Eₑ |
| 839 | E₁ₐEᵢᵢE₄ | EᵥEᵥᵢ | E₄* | Eₑ |
| 840 | E_{1b}EᵢᵢE₄ | EᵥEᵥᵢ | E₄* | Eₑ |
| 841 | E₁ₐEᵢᵢE_{iib}E₄ | EᵥEᵥᵢ | E₄* | Eₑ |
| 842 | E_{1b}EᵢᵢE_{iib}E₄ | EᵥEᵥᵢ | E₄* | Eₑ |
| 843 | E₁ₐE₅EᵢE₄ | EᵥEᵥᵢ | E₄* | Eₑ |
| 844 | E_{1b}E₅EᵢE₄ | EᵥEᵥᵢ | E₄* | Eₑ |
| 845 | E₁ₐE₅EᵢᵢE₄ | EᵥEᵥᵢ | E₄* | Eₑ |
| 846 | E_{1b}E₅EᵢᵢE₄ | EᵥEᵥᵢ | E₄* | Eₑ |
| 847 | E₁ₐE₅EᵢᵢE_{iib}E₄ | EᵥEᵥᵢ | E₄* | Eₑ |
| 848 | E_{1b}E₅EᵢᵢE_{iib}E₄ | EᵥEᵥᵢ | E₄* | Eₑ |
| 849 | E₁ₐEᵢᵢᵢ | EᵥEᵥᵢ | E₄* | Eₑ |
| 850 | E_{1b}Eᵢᵢᵢ | EᵥEᵥᵢ | E₄* | Eₑ |
| 851 | E₁ₐE₅Eᵢᵢᵢ | EᵥEᵥᵢ | E₄* | Eₑ |
| 852 | E_{1b}E₅Eᵢᵢᵢ | EᵥEᵥᵢ | E₄* | Eₑ |
| 853 | E₁ₐEᵢᵢᵢE₄ | EᵥEᵥᵢ | E₄* | Eₑ |
| 854 | E_{1b}EᵢᵢᵢE₄ | EᵥEᵥᵢ | E₄* | Eₑ |
| 855 | E₁ₐE₅EᵢᵢᵢE₄ | EᵥEᵥᵢ | E₄* | Eₑ |
| 856 | E_{1b}E₅EᵢᵢᵢE₄ | EᵥEᵥᵢ | E₄* | Eₑ |
| 857 | E₁ₐEᵢᵥ | EᵥEᵥᵢ | E₄* | Eₑ |
| 858 | E_{1b}Eᵢᵥ | EᵥEᵥᵢ | E₄* | Eₑ |
| 859 | E₁ₐE₅Eᵢᵥ | EᵥEᵥᵢ | E₄* | Eₑ |
| 860 | E_{1b}E₅Eᵢᵥ | EᵥEᵥᵢ | E₄* | Eₑ |
| 861 | E₁ₐEᵢᵥE₄ | EᵥEᵥᵢ | E₄* | Eₑ |
| 862 | E_{1b}EᵢᵥE₄ | EᵥEᵥᵢ | E₄* | Eₑ |
| 863 | E₁ₐE₅EᵢᵥE₄ | EᵥEᵥᵢ | E₄* | Eₑ |
| 864 | E_{1b}E₅EᵢᵥE₄ | EᵥEᵥᵢ | E₄* | Eₑ |
| 865 | E₁ₐ | AlkLEᵥEᵥᵢ | | Eₑ |
| 866 | E_{1b} | AlkLEᵥEᵥᵢ | | Eₑ |
| 867 | E₁ₐE₅ | AlkLEᵥEᵥᵢ | | Eₑ |
| 868 | E_{1b}E₅ | AlkLEᵥEᵥᵢ | | Eₑ |
| 869 | E₁ₐEᵢ | AlkLEᵥEᵥᵢ | | Eₑ |
| 870 | E_{1b}Eᵢ | AlkLEᵥEᵥᵢ | | Eₑ |
| 871 | E₁ₐEᵢᵢ | AlkLEᵥEᵥᵢ | | Eₑ |
| 872 | E_{1b}Eᵢᵢ | AlkLEᵥEᵥᵢ | | Eₑ |
| 873 | E₁ₐEᵢᵢE_{iib} | AlkLEᵥEᵥᵢ | | Eₑ |
| 874 | E_{1b}EᵢᵢE_{iib} | AlkLEᵥEᵥᵢ | | Eₑ |
| 875 | E₁ₐE₅Eᵢ | AlkLEᵥEᵥᵢ | | Eₑ |
| 876 | E_{1b}E₅Eᵢ | AlkLEᵥEᵥᵢ | | Eₑ |
| 877 | E₁ₐE₅Eᵢᵢ | AlkLEᵥEᵥᵢ | | Eₑ |
| 878 | E_{1b}E₅Eᵢᵢ | AlkLEᵥEᵥᵢ | | Eₑ |
| 879 | E₁ₐE₅EᵢᵢE_{iib} | AlkLEᵥEᵥᵢ | | Eₑ |
| 880 | E_{1b}E₅EᵢᵢE_{iib} | AlkLEᵥEᵥᵢ | | Eₑ |
| 881 | E₁ₐE₄ | AlkLEᵥEᵥᵢ | | Eₑ |
| 882 | E_{1b}E₄ | AlkLEᵥEᵥᵢ | | Eₑ |
| 883 | E₁ₐE₅E₄ | AlkLEᵥEᵥᵢ | | Eₑ |
| 884 | E_{1b}E₅E₄ | AlkLEᵥEᵥᵢ | | Eₑ |
| 885 | E₁ₐEᵢE₄ | AlkLEᵥEᵥᵢ | | Eₑ |
| 886 | E_{1b}EᵢE₄ | AlkLEᵥEᵥᵢ | | Eₑ |
| 887 | E₁ₐEᵢᵢE₄ | AlkLEᵥEᵥᵢ | | Eₑ |
| 888 | E_{1b}EᵢᵢE₄ | AlkLEᵥEᵥᵢ | | Eₑ |
| 889 | E₁ₐEᵢᵢE_{iib}E₄ | AlkLEᵥEᵥᵢ | | Eₑ |
| 890 | E_{1b}EᵢᵢE_{iib}E₄ | AlkLEᵥEᵥᵢ | | Eₑ |
| 891 | E₁ₐE₅EᵢE₄ | AlkLEᵥEᵥᵢ | | Eₑ |
| 892 | E_{1b}E₅EᵢE₄ | AlkLEᵥEᵥᵢ | | Eₑ |
| 893 | E₁ₐE₅EᵢᵢE₄ | AlkLEᵥEᵥᵢ | | Eₑ |
| 894 | E_{1b}E₅EᵢᵢE₄ | AlkLEᵥEᵥᵢ | | Eₑ |
| 895 | E₁ₐE₅EᵢᵢE_{iib}E₄ | AlkLEᵥEᵥᵢ | | Eₑ |
| 896 | E_{1b}E₅EᵢᵢE_{iib}E₄ | AlkLEᵥEᵥᵢ | | Eₑ |
| 897 | E₁ₐEᵢᵢᵢ | AlkLEᵥEᵥᵢ | | Eₑ |
| 898 | E_{1b}Eᵢᵢᵢ | AlkLEᵥEᵥᵢ | | Eₑ |
| 899 | E₁ₐE₅Eᵢᵢᵢ | AlkLEᵥEᵥᵢ | | Eₑ |
| 900 | E_{1b}E₅Eᵢᵢᵢ | AlkLEᵥEᵥᵢ | | Eₑ |
| 901 | E₁ₐEᵢᵢᵢE₄ | AlkLEᵥEᵥᵢ | | Eₑ |
| 902 | E_{1b}EᵢᵢᵢE₄ | AlkLEᵥEᵥᵢ | | Eₑ |
| 903 | E₁ₐE₅EᵢᵢᵢE₄ | AlkLEᵥEᵥᵢ | | Eₑ |
| 904 | E_{1b}E₅EᵢᵢᵢE₄ | AlkLEᵥEᵥᵢ | | Eₑ |
| 905 | E₁ₐEᵢᵥ | AlkLEᵥEᵥᵢ | | Eₑ |
| 906 | E_{1b}Eᵢᵥ | AlkLEᵥEᵥᵢ | | Eₑ |
| 907 | E₁ₐE₅Eᵢᵥ | AlkLEᵥEᵥᵢ | | Eₑ |
| 908 | E_{1b}E₅Eᵢᵥ | AlkLEᵥEᵥᵢ | | Eₑ |
| 909 | E₁ₐEᵢᵥE₄ | AlkLEᵥEᵥᵢ | | Eₑ |
| 910 | E_{1b}EᵢᵥE₄ | AlkLEᵥEᵥᵢ | | Eₑ |
| 911 | E₁ₐE₅EᵢᵥE₄ | AlkLEᵥEᵥᵢ | | Eₑ |
| 912 | E_{1b}E₅EᵢᵥE₄ | AlkLEᵥEᵥᵢ | | Eₑ |
| 913 | E₁ₐ | AlkLEᵥEᵥᵢ | E₄* | Eₑ |
| 914 | E_{1b} | AlkLEᵥEᵥᵢ | E₄* | Eₑ |
| 915 | E₁ₐE₅ | AlkLEᵥEᵥᵢ | E₄* | Eₑ |
| 916 | E_{1b}E₅ | AlkLEᵥEᵥᵢ | E₄* | Eₑ |
| 917 | E₁ₐEᵢ | AlkLEᵥEᵥᵢ | E₄* | Eₑ |
| 918 | E_{1b}Eᵢ | AlkLEᵥEᵥᵢ | E₄* | Eₑ |
| 919 | E₁ₐEᵢᵢ | AlkLEᵥEᵥᵢ | E₄* | Eₑ |
| 920 | E_{1b}Eᵢᵢ | AlkLEᵥEᵥᵢ | E₄* | Eₑ |
| 921 | E₁ₐEᵢᵢE_{iib} | AlkLEᵥEᵥᵢ | E₄* | Eₑ |
| 922 | E_{1b}EᵢᵢE_{iib} | AlkLEᵥEᵥᵢ | E₄* | Eₑ |
| 923 | E₁ₐE₅Eᵢ | AlkLEᵥEᵥᵢ | E₄* | Eₑ |
| 924 | E_{1b}E₅Eᵢ | AlkLEᵥEᵥᵢ | E₄* | Eₑ |
| 925 | E₁ₐE₅Eᵢᵢ | AlkLEᵥEᵥᵢ | E₄* | Eₑ |
| 926 | E_{1b}E₅Eᵢᵢ | AlkLEᵥEᵥᵢ | E₄* | Eₑ |
| 927 | E₁ₐE₅EᵢᵢE_{iib} | AlkLEᵥEᵥᵢ | E₄* | Eₑ |
| 928 | E_{1b}E₅EᵢᵢE_{iib} | AlkLEᵥEᵥᵢ | E₄* | Eₑ |
| 929 | E₁ₐE₄ | AlkLEᵥEᵥᵢ | E₄* | Eₑ |
| 930 | E_{1b}E₄ | AlkLEᵥEᵥᵢ | E₄* | Eₑ |
| 931 | E₁ₐE₅E₄ | AlkLEᵥEᵥᵢ | E₄* | Eₑ |
| 932 | E_{1b}E₅E₄ | AlkLEᵥEᵥᵢ | E₄* | Eₑ |
| 933 | E₁ₐEᵢE₄ | AlkLEᵥEᵥᵢ | E₄* | Eₑ |
| 934 | E_{1b}EᵢE₄ | AlkLEᵥEᵥᵢ | E₄* | Eₑ |
| 935 | E₁ₐEᵢᵢE₄ | AlkLEᵥEᵥᵢ | E₄* | Eₑ |
| 936 | E_{1b}EᵢᵢE₄ | AlkLEᵥEᵥᵢ | E₄* | Eₑ |
| 937 | E₁ₐEᵢᵢE_{iib}E₄ | AlkLEᵥEᵥᵢ | E₄* | Eₑ |
| 938 | E_{1b}EᵢᵢE_{iib}E₄ | AlkLEᵥEᵥᵢ | E₄* | Eₑ |
| 939 | E₁ₐE₅EᵢE₄ | AlkLEᵥEᵥᵢ | E₄* | Eₑ |
| 940 | E_{1b}E₅EᵢE₄ | AlkLEᵥEᵥᵢ | E₄* | Eₑ |
| 941 | E₁ₐE₅EᵢᵢE₄ | AlkLEᵥEᵥᵢ | E₄* | Eₑ |
| 942 | E_{1b}E₅EᵢᵢE₄ | AlkLEᵥEᵥᵢ | E₄* | Eₑ |
| 943 | E₁ₐE₅EᵢᵢE_{iib}E₄ | AlkLEᵥEᵥᵢ | E₄* | Eₑ |
| 944 | E_{1b}E₅EᵢᵢE_{iib}E₄ | AlkLEᵥEᵥᵢ | E₄* | Eₑ |
| 945 | E₁ₐEᵢᵢᵢ | AlkLEᵥEᵥᵢ | E₄* | Eₑ |
| 946 | E_{1b}Eᵢᵢᵢ | AlkLEᵥEᵥᵢ | E₄* | Eₑ |
| 947 | E₁ₐE₅Eᵢᵢᵢ | AlkLEᵥEᵥᵢ | E₄* | Eₑ |
| 948 | E_{1b}E₅Eᵢᵢᵢ | AlkLEᵥEᵥᵢ | E₄* | Eₑ |
| 949 | E₁ₐEᵢᵢᵢE₄ | AlkLEᵥEᵥᵢ | E₄* | Eₑ |
| 950 | E_{1b}EᵢᵢᵢE₄ | AlkLEᵥEᵥᵢ | E₄* | Eₑ |
| 951 | E₁ₐE₅EᵢᵢᵢE₄ | AlkLEᵥEᵥᵢ | E₄* | Eₑ |
| 952 | E_{1b}E₅EᵢᵢᵢE₄ | AlkLEᵥEᵥᵢ | E₄* | Eₑ |
| 953 | E₁ₐEᵢᵥ | AlkLEᵥEᵥᵢ | E₄* | Eₑ |
| 954 | E_{1b}Eᵢᵥ | AlkLEᵥEᵥᵢ | E₄* | Eₑ |
| 955 | E₁ₐE₅Eᵢᵥ | AlkLEᵥEᵥᵢ | E₄* | Eₑ |
| 956 | E_{1b}E₅Eᵢᵥ | AlkLEᵥEᵥᵢ | E₄* | Eₑ |
| 957 | E₁ₐEᵢᵥE₄ | AlkLEᵥEᵥᵢ | E₄* | Eₑ |
| 958 | E_{1b}EᵢᵥE₄ | AlkLEᵥEᵥᵢ | E₄* | Eₑ |
| 959 | E₁ₐE₅EᵢᵥE₄ | AlkLEᵥEᵥᵢ | E₄* | Eₑ |
| 960 | E_{1b}E₅EᵢᵥE₄ | AlkLEᵥEᵥᵢ | E₄* | Eₑ |
| 961 | E₁ₐ | EᵥEᵥᵢ | | E_{f} |
| 962 | E_{1b} | EᵥEᵥᵢ | | E_{f} |
| 963 | E₁ₐE₅ | EᵥEᵥᵢ | | E_{f} |
| 964 | E_{1b}E₅ | EᵥEᵥᵢ | | E_{f} |
| 965 | E₁ₐEᵢ | EᵥEᵥᵢ | | E_{f} |
| 966 | E_{1b}Eᵢ | EᵥEᵥᵢ | | E_{f} |
| 967 | E₁ₐEᵢᵢ | EᵥEᵥᵢ | | E_{f} |
| 968 | E_{1b}Eᵢᵢ | EᵥEᵥᵢ | | E_{f} |
| 969 | E₁ₐEᵢᵢE_{iib} | EᵥEᵥᵢ | | E_{f} |
| 970 | E_{1b}EᵢᵢE_{iib} | EᵥEᵥᵢ | | E_{f} |
| 971 | E₁ₐE₅Eᵢ | EᵥEᵥᵢ | | E_{f} |
| 972 | E_{1b}E₅Eᵢ | EᵥEᵥᵢ | | E_{f} |
| 973 | E₁ₐE₅Eᵢᵢ | EᵥEᵥᵢ | | E_{f} |
| 974 | E_{1b}E₅Eᵢᵢ | EᵥEᵥᵢ | | E_{f} |
| 975 | E₁ₐE₅EᵢᵢE_{iib} | EᵥEᵥᵢ | | E_{f} |
| 976 | E_{1b}E₅EᵢᵢE_{iib} | EᵥEᵥᵢ | | E_{f} |
| 977 | E₁ₐE₄ | EᵥEᵥᵢ | | E_{f} |
| 978 | E_{1b}E₄ | EᵥEᵥᵢ | | E_{f} |
| 979 | E₁ₐE₅E₄ | EᵥEᵥᵢ | | E_{f} |
| 980 | E_{1b}E₅E₄ | EᵥEᵥᵢ | | E_{f} |
| 981 | E₁ₐEᵢE₄ | EᵥEᵥᵢ | | E_{f} |
| 982 | E_{1b}EᵢE₄ | EᵥEᵥᵢ | | E_{f} |
| 983 | E₁ₐEᵢᵢE₄ | EᵥEᵥᵢ | | E_{f} |
| 984 | E_{1b}EᵢᵢE₄ | EᵥEᵥᵢ | | E_{f} |
| 985 | E₁ₐEᵢᵢE_{iib}E₄ | EᵥEᵥᵢ | | E_{f} |
| 986 | E_{1b}EᵢᵢE_{iib}E₄ | EᵥEᵥᵢ | | E_{f} |
| 987 | E₁ₐE₅EᵢE₄ | EᵥEᵥᵢ | | E_{f} |
| 988 | E_{1b}E₅EᵢE₄ | EᵥEᵥᵢ | | E_{f} |
| 989 | E₁ₐE₅EᵢᵢE₄ | EᵥEᵥᵢ | | E_{f} |
| 990 | E_{1b}E₅EᵢᵢE₄ | EᵥEᵥᵢ | | E_{f} |
| 991 | E₁ₐE₅EᵢᵢE_{iib}E₄ | EᵥEᵥᵢ | | E_{f} |
| 992 | E_{1b}E₅EᵢᵢE_{iib}E₄ | EᵥEᵥᵢ | | E_{f} |
| 993 | E₁ₐEᵢᵢᵢ | EᵥEᵥᵢ | | E_{f} |
| 994 | E_{1b}Eᵢᵢᵢ | EᵥEᵥᵢ | | E_{f} |
| 995 | E₁ₐE₅Eᵢᵢᵢ | EᵥEᵥᵢ | | E_{f} |
| 996 | E_{1b}E₅Eᵢᵢᵢ | EᵥEᵥᵢ | | E_{f} |
| 997 | E₁ₐEᵢᵢᵢE₄ | EᵥEᵥᵢ | | E_{f} |
| 998 | E_{1b}EᵢᵢᵢE₄ | EᵥEᵥᵢ | | E_{f} |
| 999 | E₁ₐE₅EᵢᵢᵢE₄ | EᵥEᵥᵢ | | E_{f} |
| 1000 | E_{1b}E₅EᵢᵢᵢE₄ | EᵥEᵥᵢ | | E_{f} |
| 1001 | E₁ₐEᵢᵥ | EᵥEᵥᵢ | | E_{f} |
| 1002 | E_{1b}Eᵢᵥ | EᵥEᵥᵢ | | E_{f} |
| 1003 | E₁ₐE₅Eᵢᵥ | EᵥEᵥᵢ | | E_{f} |
| 1004 | E_{1b}E₅Eᵢᵥ | EᵥEᵥᵢ | | E_{f} |
| 1005 | E₁ₐEᵢᵥE₄ | EᵥEᵥᵢ | | E_{f} |
| 1006 | E_{1b}EᵢᵥE₄ | EᵥEᵥᵢ | | E_{f} |
| 1007 | E₁ₐE₅EᵢᵥE₄ | EᵥEᵥᵢ | | E_{f} |
| 1008 | E_{1b}E₅EᵢᵥE₄ | EᵥEᵥᵢ | | E_{f} |
| 1009 | E₁ₐ | EᵥEᵥᵢ | E₄* | E_{f} |
| 1010 | E_{1b} | EᵥEᵥᵢ | E₄* | E_{f} |
| 1011 | E₁ₐE₅ | EᵥEᵥᵢ | E₄* | E_{f} |
| 1012 | E_{1b}E₅ | EᵥEᵥᵢ | E₄* | E_{f} |
| 1013 | E₁ₐEᵢ | EᵥEᵥᵢ | E₄* | E_{f} |
| 1014 | E_{1b}Eᵢ | EᵥEᵥᵢ | E₄* | E_{f} |
| 1015 | E₁ₐEᵢᵢ | EᵥEᵥᵢ | E₄* | E_{f} |
| 1016 | E_{1b}Eᵢᵢ | EᵥEᵥᵢ | E₄* | E_{f} |
| 1017 | E₁ₐEᵢᵢE_{iib} | EᵥEᵥᵢ | E₄* | E_{f} |
| 1018 | E_{1b}EᵢᵢE_{iib} | EᵥEᵥᵢ | E₄* | E_{f} |
| 1019 | E₁ₐE₅Eᵢ | EᵥEᵥᵢ | E₄* | E_{f} |
| 1020 | E_{1b}E₅Eᵢ | EᵥEᵥᵢ | E₄* | E_{f} |
| 1021 | E₁ₐE₅Eᵢᵢ | EᵥEᵥᵢ | E₄* | E_{f} |
| 1022 | E_{1b}E₅Eᵢᵢ | EᵥEᵥᵢ | E₄* | E_{f} |
| 1023 | E₁ₐE₅EᵢᵢE_{iib} | EᵥEᵥᵢ | E₄* | E_{f} |
| 1024 | E_{1b}E₅EᵢᵢE_{iib} | EᵥEᵥᵢ | E₄* | E_{f} |
| 1025 | E₁ₐE₄ | EᵥEᵥᵢ | E₄* | E_{f} |
| 1026 | E_{1b}E₄ | EᵥEᵥᵢ | E₄* | E_{f} |
| 1027 | E₁ₐE₅E₄ | EᵥEᵥᵢ | E₄* | E_{f} |
| 1028 | E_{1b}E₅E₄ | EᵥEᵥᵢ | E₄* | E_{f} |
| 1029 | E₁ₐEᵢE₄ | EᵥEᵥᵢ | E₄* | E_{f} |
| 1030 | E_{1b}EᵢE₄ | EᵥEᵥᵢ | E₄* | E_{f} |
| 1031 | E₁ₐEᵢᵢE₄ | EᵥEᵥᵢ | E₄* | E_{f} |
| 1032 | E_{1b}EᵢᵢE₄ | EᵥEᵥᵢ | E₄* | E_{f} |
| 1033 | E₁ₐEᵢᵢE_{iib}E₄ | EᵥEᵥᵢ | E₄* | E_{f} |
| 1034 | E_{1b}EᵢᵢE_{iib}E₄ | EᵥEᵥᵢ | E₄* | E_{f} |
| 1035 | E₁ₐE₅EᵢE₄ | EᵥEᵥᵢ | E₄* | E_{f} |
| 1036 | E_{1b}E₅EᵢE₄ | EᵥEᵥᵢ | E₄* | E_{f} |
| 1037 | E₁ₐE₅EᵢᵢE₄ | EᵥEᵥᵢ | E₄* | E_{f} |
| 1038 | E_{1b}E₅EᵢᵢE₄ | EᵥEᵥᵢ | E₄* | E_{f} |
| 1039 | E₁ₐE₅EᵢᵢE_{iib}E₄ | EᵥEᵥᵢ | E₄* | E_{f} |
| 1040 | E_{1b}E₅EᵢᵢE_{iib}E₄ | EᵥEᵥᵢ | E₄* | E_{f} |
| 1041 | E₁ₐEᵢᵢᵢ | EᵥEᵥᵢ | E₄* | E_{f} |
| 1042 | E_{1b}Eᵢᵢᵢ | EᵥEᵥᵢ | E₄* | E_{f} |
| 1043 | E₁ₐE₅Eᵢᵢᵢ | EᵥEᵥᵢ | E₄* | E_{f} |
| 1044 | E_{1b}E₅Eᵢᵢᵢ | EᵥEᵥᵢ | E₄* | E_{f} |
| 1045 | E₁ₐEᵢᵢᵢE₄ | EᵥEᵥᵢ | E₄* | E_{f} |
| 1046 | E_{1b}EᵢᵢᵢE₄ | EᵥEᵥᵢ | E₄* | E_{f} |
| 1047 | E₁ₐE₅EᵢᵢᵢE₄ | EᵥEᵥᵢ | E₄* | E_{f} |
| 1048 | E_{1b}E₅EᵢᵢᵢE₄ | EᵥEᵥᵢ | E₄* | E_{f} |
| 1049 | E₁ₐEᵢᵥ | EᵥEᵥᵢ | E₄* | E_{f} |
| 1050 | E_{1b}Eᵢᵥ | EᵥEᵥᵢ | E₄* | E_{f} |
| 1051 | E₁ₐE₅Eᵢᵥ | EᵥEᵥᵢ | E₄* | E_{f} |
| 1052 | E_{1b}E₅Eᵢᵥ | EᵥEᵥᵢ | E₄* | E_{f} |
| 1053 | E₁ₐEᵢᵥE₄ | EᵥEᵥᵢ | E₄* | E_{f} |
| 1054 | E_{1b}EᵢᵥE₄ | EᵥEᵥᵢ | E₄* | E_{f} |
| 1055 | E₁ₐE₅EᵢᵥE₄ | EᵥEᵥᵢ | E₄* | E_{f} |
| 1056 | E_{1b}E₅EᵢᵥE₄ | EᵥEᵥᵢ | E₄* | E_{f} |
| 1057 | E₁ₐ | AlkLEᵥEᵥᵢ | | E_{f} |
| 1058 | E_{1b} | AlkLEᵥEᵥᵢ | | E_{f} |
| 1059 | E₁ₐE₅ | AlkLEᵥEᵥᵢ | | E_{f} |
| 1060 | E_{1b}E₅ | AlkLEᵥEᵥᵢ | | E_{f} |
| 1061 | E₁ₐEᵢ | AlkLEᵥEᵥᵢ | | E_{f} |
| 1062 | E_{1b}Eᵢ | AlkLEᵥEᵥᵢ | | E_{f} |
| 1063 | E₁ₐEᵢᵢ | AlkLEᵥEᵥᵢ | | E_{f} |
| 1064 | E_{1b}Eᵢᵢ | AlkLEᵥEᵥᵢ | | E_{f} |
| 1065 | E₁ₐEᵢᵢE_{iib} | AlkLEᵥEᵥᵢ | | E_{f} |
| 1066 | E_{1b}EᵢᵢE_{iib} | AlkLEᵥEᵥᵢ | | E_{f} |
| 1067 | E₁ₐE₅Eᵢ | AlkLEᵥEᵥᵢ | | E_{f} |
| 1068 | E_{1b}E₅Eᵢ | AlkLEᵥEᵥᵢ | | E_{f} |
| 1069 | E₁ₐE₅Eᵢᵢ | AlkLEᵥEᵥᵢ | | E_{f} |
| 1070 | E_{1b}E₅Eᵢᵢ | AlkLEᵥEᵥᵢ | | E_{f} |
| 1071 | E₁ₐE₅EᵢᵢE_{iib} | AlkLEᵥEᵥᵢ | | E_{f} |
| 1072 | E_{1b}E₅EᵢᵢE_{iib} | AlkLEᵥEᵥᵢ | | E_{f} |
| 1073 | E₁ₐE₄ | AlkLEᵥEᵥᵢ | | E_{f} |
| 1074 | E_{1b}E₄ | AlkLEᵥEᵥᵢ | | E_{f} |
| 1075 | E₁ₐE₅E₄ | AlkLEᵥEᵥᵢ | | E_{f} |
| 1076 | E_{1b}E₅E₄ | AlkLEᵥEᵥᵢ | | E_{f} |
| 1077 | E₁ₐEᵢE₄ | AlkLEᵥEᵥᵢ | | E_{f} |
| 1078 | E_{1b}EᵢE₄ | AlkLEᵥEᵥᵢ | | E_{f} |
| 1079 | E₁ₐEᵢᵢE₄ | AlkLEᵥEᵥᵢ | | E_{f} |
| 1080 | E_{1b}EᵢᵢE₄ | AlkLEᵥEᵥᵢ | | E_{f} |
| 1081 | E₁ₐEᵢᵢE_{iib}E₄ | AlkLEᵥEᵥᵢ | | E_{f} |
| 1082 | E_{1b}EᵢᵢE_{iib}E₄ | AlkLEᵥEᵥᵢ | | E_{f} |
| 1083 | E₁ₐE₅EᵢE₄ | AlkLEᵥEᵥᵢ | | E_{f} |
| 1084 | E_{1b}E₅EᵢE₄ | AlkLEᵥEᵥᵢ | | E_{f} |
| 1085 | E₁ₐE₅EᵢᵢE₄ | AlkLEᵥEᵥᵢ | | E_{f} |
| 1086 | E_{1b}E₅EᵢᵢE₄ | AlkLEᵥEᵥᵢ | | E_{f} |
| 1087 | E₁ₐE₅EᵢᵢE_{iib}E₄ | AlkLEᵥEᵥᵢ | | E_{f} |
| 1088 | E_{1b}E₅EᵢᵢE_{iib}E₄ | AlkLEᵥEᵥᵢ | | E_{f} |
| 1089 | E₁ₐEᵢᵢᵢ | AlkLEᵥEᵥᵢ | | E_{f} |
| 1090 | E_{1b}Eᵢᵢᵢ | AlkLEᵥEᵥᵢ | | E_{f} |
| 1091 | E₁ₐE₅Eᵢᵢᵢ | AlkLEᵥEᵥᵢ | | E_{f} |
| 1092 | E_{1b}E₅Eᵢᵢᵢ | AlkLEᵥEᵥᵢ | | E_{f} |
| 1093 | E₁ₐEᵢᵢᵢE₄ | AlkLEᵥEᵥᵢ | | E_{f} |
| 1094 | E_{1b}EᵢᵢᵢE₄ | AlkLEᵥEᵥᵢ | | E_{f} |
| 1095 | E₁ₐE₅EᵢᵢᵢE₄ | AlkLEᵥEᵥᵢ | | E_{f} |
| 1096 | E_{1b}E₅EᵢᵢᵢE₄ | AlkLEᵥEᵥᵢ | | E_{f} |
| 1097 | E₁ₐEᵢᵥ | AlkLEᵥEᵥᵢ | | E_{f} |
| 1098 | E_{1b}Eᵢᵥ | AlkLEᵥEᵥᵢ | | E_{f} |
| 1099 | E₁ₐE₅Eᵢᵥ | AlkLEᵥEᵥᵢ | | E_{f} |
| 1100 | E_{1b}E₅Eᵢᵥ | AlkLEᵥEᵥᵢ | | E_{f} |
| 1101 | E₁ₐEᵢᵥE₄ | AlkLEᵥEᵥᵢ | | E_{f} |
| 1102 | E_{1b}EᵢᵥE₄ | AlkLEᵥEᵥᵢ | | E_{f} |
| 1103 | E₁ₐE₅EᵢᵥE₄ | AlkLEᵥEᵥᵢ | | E_{f} |
| 1104 | E_{1b}E₅EᵢᵥE₄ | AlkLEᵥEᵥᵢ | | E_{f} |
| 1105 | E₁ₐ | AlkLEᵥEᵥᵢ | E₄* | E_{f} |
| 1106 | E_{1b} | AlkLEᵥEᵥᵢ | E₄* | E_{f} |
| 1107 | E₁ₐE₅ | AlkLEᵥEᵥᵢ | E₄* | E_{f} |
| 1108 | E_{1b}E₅ | AlkLEᵥEᵥᵢ | E₄* | E_{f} |
| 1109 | E₁ₐEᵢ | AlkLEᵥEᵥᵢ | E₄* | E_{f} |
| 1110 | E_{1b}Eᵢ | AlkLEᵥEᵥᵢ | E₄* | E_{f} |
| 1111 | E₁ₐEᵢᵢ | AlkLEᵥEᵥᵢ | E₄* | E_{f} |
| 1112 | E_{1b}Eᵢᵢ | AlkLEᵥEᵥᵢ | E₄* | E_{f} |
| 1113 | E₁ₐEᵢᵢE_{iib} | AlkLEᵥEᵥᵢ | E₄* | E_{f} |
| 1114 | E_{1b}EᵢᵢE_{iib} | AlkLEᵥEᵥᵢ | E₄* | E_{f} |
| 1115 | E₁ₐE₅Eᵢ | AlkLEᵥEᵥᵢ | E₄* | E_{f} |
| 1116 | E_{1b}E₅Eᵢ | AlkLEᵥEᵥᵢ | E₄* | E_{f} |
| 1117 | E₁ₐE₅Eᵢᵢ | AlkLEᵥEᵥᵢ | E₄* | E_{f} |
| 1118 | E_{1b}E₅Eᵢᵢ | AlkLEᵥEᵥᵢ | E₄* | E_{f} |
| 1119 | E₁ₐE₅EᵢᵢE_{iib} | AlkLEᵥEᵥᵢ | E₄* | E_{f} |
| 1120 | E_{1b}E₅EᵢᵢE_{iib} | AlkLEᵥEᵥᵢ | E₄* | E_{f} |
| 1121 | E₁ₐE₄ | AlkLEᵥEᵥᵢ | E₄* | E_{f} |
| 1122 | E_{1b}E₄ | AlkLEᵥEᵥᵢ | E₄* | E_{f} |
| 1123 | E₁ₐE₅E₄ | AlkLEᵥEᵥᵢ | E₄* | E_{f} |
| 1124 | E_{1b}E₅E₄ | AlkLEᵥEᵥᵢ | E₄* | E_{f} |
| 1125 | E₁ₐEᵢE₄ | AlkLEᵥEᵥᵢ | E₄* | E_{f} |
| 1126 | E_{1b}EᵢE₄ | AlkLEᵥEᵥᵢ | E₄* | E_{f} |
| 1127 | E₁ₐEᵢᵢE₄ | AlkLEᵥEᵥᵢ | E₄* | E_{f} |
| 1128 | E_{1b}EᵢᵢE₄ | AlkLEᵥEᵥᵢ | E₄* | E_{f} |
| 1129 | E₁ₐEᵢᵢE_{iib}E₄ | AlkLEᵥEᵥᵢ | E₄* | E_{f} |
| 1130 | E_{1b}EᵢᵢE_{iib}E₄ | AlkLEᵥEᵥᵢ | E₄* | E_{f} |
| 1131 | E₁ₐE₅EᵢE₄ | AlkLEᵥEᵥᵢ | E₄* | E_{f} |
| 1132 | E_{1b}E₅EᵢE₄ | AlkLEᵥEᵥᵢ | E₄* | E_{f} |
| 1133 | E₁ₐE₅EᵢᵢE₄ | AlkLEᵥEᵥᵢ | E₄* | E_{f} |
| 1134 | E_{1b}E₅EᵢᵢE₄ | AlkLEᵥEᵥᵢ | E₄* | E_{f} |
| 1135 | E₁ₐE₅EᵢᵢE_{iib}E₄ | AlkLEᵥEᵥᵢ | E₄* | E_{f} |
| 1136 | E_{1b}E₅EᵢᵢE_{iib}E₄ | AlkLEᵥEᵥᵢ | E₄* | E_{f} |
| 1137 | E₁ₐEᵢᵢᵢ | AlkLEᵥEᵥᵢ | E₄* | E_{f} |
| 1138 | E_{1b}Eᵢᵢᵢ | AlkLEᵥEᵥᵢ | E₄* | E_{f} |
| 1139 | E₁ₐE₅Eᵢᵢᵢ | AlkLEᵥEᵥᵢ | E₄* | E_{f} |
| 1140 | E_{1b}E₅Eᵢᵢᵢ | AlkLEᵥEᵥᵢ | E₄* | E_{f} |
| 1141 | E₁ₐEᵢᵢᵢE₄ | AlkLEᵥEᵥᵢ | E₄* | E_{f} |
| 1142 | E_{1b}EᵢᵢᵢE₄ | AlkLEᵥEᵥᵢ | E₄* | E_{f} |
| 1143 | E₁ₐE₅EᵢᵢᵢE₄ | AlkLEᵥEᵥᵢ | E₄* | E_{f} |
| 1144 | E_{1b}E₅EᵢᵢᵢE₄ | AlkLEᵥEᵥᵢ | E₄* | E_{f} |
| 1145 | E₁ₐEᵢᵥ | AlkLEᵥEᵥᵢ | E₄* | E_{f} |
| 1146 | E_{1b}Eᵢᵥ | AlkLEᵥEᵥᵢ | E₄* | E_{f} |
| 1147 | E₁ₐE₅Eᵢᵥ | AlkLEᵥEᵥᵢ | E₄* | E_{f} |
| 1148 | E_{1b}E₅Eᵢᵥ | AlkLEᵥEᵥᵢ | E₄* | E_{f} |
| 1149 | E₁ₐEᵢᵥE₄ | AlkLEᵥEᵥᵢ | E₄* | E_{f} |
| 1150 | E_{1b}EᵢᵥE₄ | AlkLEᵥEᵥᵢ | E₄* | E_{f} |
| 1151 | E₁ₐE₅EᵢᵥE₄ | AlkLEᵥEᵥᵢ | E₄* | E_{f} |
| 1152 | E_{1b}E₅EᵢᵥE₄ | AlkLEᵥEᵥᵢ | E₄* | E_{f} |
| 1153 | E₁ₐ | Eᵥᵢᵢ | | |
| 1154 | E_{1b} | Eᵥᵢᵢ | | |
| 1155 | E₁ₐE₅ | Eᵥᵢᵢ | | |
| 1156 | E_{1b}E₅ | Eᵥᵢᵢ | | |
| 1157 | E₁ₐEᵢ | Eᵥᵢᵢ | | |
| 1158 | E_{1b}Eᵢ | Eᵥᵢᵢ | | |
| 1159 | E₁ₐEᵢᵢ | Eᵥᵢᵢ | | |
| 1160 | E_{1b}Eᵢᵢ | Eᵥᵢᵢ | | |
| 1161 | E₁ₐEᵢᵢE_{iib} | Eᵥᵢᵢ | | |
| 1162 | E_{1b}EᵢᵢE_{iib} | Eᵥᵢᵢ | | |
| 1163 | E₁ₐE₅Eᵢ | Eᵥᵢᵢ | | |
| 1164 | E_{1b}E₅Eᵢ | Eᵥᵢᵢ | | |
| 1165 | E₁ₐE₅Eᵢᵢ | Eᵥᵢᵢ | | |
| 1166 | E_{1b}E₅Eᵢᵢ | Eᵥᵢᵢ | | |
| 1167 | E₁ₐE₅EᵢᵢE_{iib} | Eᵥᵢᵢ | | |
| 1168 | E_{1b}E₅EᵢᵢE_{iib} | Eᵥᵢᵢ | | |
| 1169 | E₁ₐE₄ | Eᵥᵢᵢ | | |
| 1170 | E_{1b}E₄ | Eᵥᵢᵢ | | |
| 1171 | E₁ₐE₅E₄ | Eᵥᵢᵢ | | |
| 1172 | E_{1b}E₅E₄ | Eᵥᵢᵢ | | |
| 1173 | E₁ₐEᵢE₄ | Eᵥᵢᵢ | | |
| 1174 | E_{1b}EᵢE₄ | Eᵥᵢᵢ | | |
| 1175 | E₁ₐEᵢᵢE₄ | Eᵥᵢᵢ | | |
| 1176 | E_{1b}EᵢᵢE₄ | Eᵥᵢᵢ | | |
| 1177 | E₁ₐEᵢᵢE_{iib}E₄ | Eᵥᵢᵢ | | |
| 1178 | E_{1b}EᵢᵢE_{iib}E₄ | Eᵥᵢᵢ | | |
| 1179 | E₁ₐE₅EᵢE₄ | Eᵥᵢᵢ | | |
| 1180 | E_{1b}E₅EᵢE₄ | Eᵥᵢᵢ | | |
| 1181 | E₁ₐE₅EᵢᵢE₄ | Eᵥᵢᵢ | | |
| 1182 | E_{1b}E₅EᵢᵢE₄ | Eᵥᵢᵢ | | |
| 1183 | E₁ₐE₅EᵢᵢE_{iib}E₄ | Eᵥᵢᵢ | | |
| 1184 | E_{1b}E₅EᵢᵢE_{iib}E₄ | Eᵥᵢᵢ | | |
| 1185 | E₁ₐEᵢᵢᵢ | Eᵥᵢᵢ | | |
| 1186 | E_{1b}Eᵢᵢᵢ | Eᵥᵢᵢ | | |
| 1187 | E₁ₐE₅Eᵢᵢᵢ | Eᵥᵢᵢ | | |
| 1188 | E_{1b}E₅Eᵢᵢᵢ | Eᵥᵢᵢ | | |
| 1189 | E₁ₐEᵢᵢᵢE₄ | Eᵥᵢᵢ | | |
| 1190 | E_{1b}EᵢᵢᵢE₄ | Eᵥᵢᵢ | | |
| 1191 | E₁ₐE₅EᵢᵢᵢE₄ | Eᵥᵢᵢ | | |
| 1192 | E_{1b}E₅EᵢᵢᵢE₄ | Eᵥᵢᵢ | | |
| 1193 | E₁ₐEᵢᵥ | Eᵥᵢᵢ | | |
| 1194 | E_{1b}Eᵢᵥ | Eᵥᵢᵢ | | |
| 1195 | E₁ₐE₅Eᵢᵥ | Eᵥᵢᵢ | | |
| 1196 | E_{1b}E₅Eᵢᵥ | Eᵥᵢᵢ | | |
| 1197 | E₁ₐEᵢᵥE₄ | Eᵥᵢᵢ | | |
| 1198 | E_{1b}EᵢᵥE₄ | Eᵥᵢᵢ | | |
| 1199 | E₁ₐE₅EᵢᵥE₄ | Eᵥᵢᵢ | | |
| 1200 | E_{1b}E₅EᵢᵥE₄ | Eᵥᵢᵢ | | |
| 1201 | E₁ₐ | Eᵥᵢᵢ | E₄* | |
| 1202 | E_{1b} | Eᵥᵢᵢ | E₄* | |
| 1203 | E₁ₐE₅ | Eᵥᵢᵢ | E₄* | |
| 1204 | E_{1b}E₅ | Eᵥᵢᵢ | E₄* | |
| 1205 | E₁ₐEᵢ | Eᵥᵢᵢ | E₄* | |
| 1206 | E_{1b}Eᵢ | Eᵥᵢᵢ | E₄* | |
| 1207 | E₁ₐEᵢᵢ | Eᵥᵢᵢ | E₄* | |
| 1208 | E_{1b}Eᵢᵢ | Eᵥᵢᵢ | E₄* | |
| 1209 | E₁ₐEᵢᵢE_{iib} | Eᵥᵢᵢ | E₄* | |
| 1210 | E_{1b}EᵢᵢE_{iib} | Eᵥᵢᵢ | E₄* | |
| 1211 | E₁ₐE₅Eᵢ | Eᵥᵢᵢ | E₄* | |
| 1212 | E_{1b}E₅Eᵢ | Eᵥᵢᵢ | E₄* | |
| 1213 | E₁ₐE₅Eᵢᵢ | Eᵥᵢᵢ | E₄* | |
| 1214 | E_{1b}E₅Eᵢᵢ | Eᵥᵢᵢ | E₄* | |
| 1215 | E₁ₐE₅EᵢᵢE_{iib} | Eᵥᵢᵢ | E₄* | |
| 1216 | E_{1b}E₅EᵢᵢE_{iib} | Eᵥᵢᵢ | E₄* | |
| 1217 | E₁ₐE₄ | Eᵥᵢᵢ | E₄* | |
| 1218 | E_{1b}E₄ | Eᵥᵢᵢ | E₄* | |
| 1219 | E₁ₐE₅E₄ | Eᵥᵢᵢ | E₄* | |
| 1220 | E_{1b}E₅E₄ | Eᵥᵢᵢ | E₄* | |
| 1221 | E₁ₐEᵢE₄ | Eᵥᵢᵢ | E₄* | |
| 1222 | E_{1b}EᵢE₄ | Eᵥᵢᵢ | E₄* | |
| 1223 | E₁ₐEᵢᵢE₄ | Eᵥᵢᵢ | E₄* | |
| 1224 | E_{1b}EᵢᵢE₄ | Eᵥᵢᵢ | E₄* | |
| 1225 | E₁ₐEᵢᵢE_{iib}E₄ | Eᵥᵢᵢ | E₄* | |
| 1226 | E_{1b}EᵢᵢE_{iib}E₄ | Eᵥᵢᵢ | E₄* | |
| 1227 | E₁ₐE₅EᵢE₄ | Eᵥᵢᵢ | E₄* | |
| 1228 | E_{1b}E₅EᵢE₄ | Eᵥᵢᵢ | E₄* | |
| 1229 | E₁ₐE₅EᵢᵢE₄ | Eᵥᵢᵢ | E₄* | |
| 1230 | E_{1b}E₅EᵢᵢE₄ | Eᵥᵢᵢ | E₄* | |
| 1231 | E₁ₐE₅EᵢᵢE_{iib}E₄ | Eᵥᵢᵢ | E₄* | |
| 1232 | E_{1b}E₅EᵢᵢE_{iib}E₄ | Eᵥᵢᵢ | E₄* | |
| 1233 | E₁ₐEᵢᵢᵢ | Eᵥᵢᵢ | E₄* | |
| 1234 | E_{1b}Eᵢᵢᵢ | Eᵥᵢᵢ | E₄* | |
| 1235 | E₁ₐE₅Eᵢᵢᵢ | Eᵥᵢᵢ | E₄* | |
| 1236 | E_{1b}E₅Eᵢᵢᵢ | Eᵥᵢᵢ | E₄* | |
| 1237 | E₁ₐEᵢᵢᵢE₄ | Eᵥᵢᵢ | E₄* | |
| 1238 | E_{1b}EᵢᵢᵢE₄ | Eᵥᵢᵢ | E₄* | |
| 1239 | E₁ₐE₅EᵢᵢᵢE₄ | Eᵥᵢᵢ | E₄* | |
| 1240 | E_{1b}E₅EᵢᵢᵢE₄ | Eᵥᵢᵢ | E₄* | |
| 1241 | E₁ₐEᵢᵥ | Eᵥᵢᵢ | E₄* | |
| 1242 | E_{1b}Eᵢᵥ | Eᵥᵢᵢ | E₄* | |
| 1243 | E₁ₐE₅Eᵢᵥ | Eᵥᵢᵢ | E₄* | |
| 1244 | E_{1b}E₅Eᵢᵥ | Eᵥᵢᵢ | E₄* | |
| 1245 | E₁ₐEᵢᵥE₄ | Eᵥᵢᵢ | E₄* | |
| 1246 | E_{1b}EᵢᵥE₄ | Eᵥᵢᵢ | E₄* | |
| 1247 | E₁ₐE₅EᵢᵥE₄ | Eᵥᵢᵢ | E₄* | |
| 1248 | E_{1b}E₅EᵢᵥE₄ | Eᵥᵢᵢ | E₄* | |
| 1249 | E₁ₐ | AlkLEᵥᵢᵢ | | |
| 1250 | E_{1b} | AlkLEᵥᵢᵢ | | |
| 1251 | E₁ₐE₅ | AlkLEᵥᵢᵢ | | |
| 1252 | E_{1b}E₅ | AlkLEᵥᵢᵢ | | |
| 1253 | E₁ₐEᵢ | AlkLEᵥᵢᵢ | | |
| 1254 | E_{1b}Eᵢ | AlkLEᵥᵢᵢ | | |
| 1255 | E₁ₐEᵢᵢ | AlkLEᵥᵢᵢ | | |
| 1256 | E_{1b}Eᵢᵢ | AlkLEᵥᵢᵢ | | |
| 1257 | E₁ₐEᵢᵢE_{iib} | AlkLEᵥᵢᵢ | | |
| 1258 | E_{1b}EᵢᵢE_{iib} | AlkLEᵥᵢᵢ | | |
| 1259 | E₁ₐE₅Eᵢ | AlkLEᵥᵢᵢ | | |
| 1260 | E_{1b}E₅Eᵢ | AlkLEᵥᵢᵢ | | |
| 1261 | E₁ₐE₅Eᵢᵢ | AlkLEᵥᵢᵢ | | |
| 1262 | E_{1b}E₅Eᵢᵢ | AlkLEᵥᵢᵢ | | |
| 1263 | E₁ₐE₅EᵢᵢE_{iib} | AlkLEᵥᵢᵢ | | |
| 1264 | E_{1b}E₅EᵢᵢE_{iib} | AlkLEᵥᵢᵢ | | |
| 1265 | E₁ₐE₄ | AlkLEᵥᵢᵢ | | |
| 1266 | E_{1b}E₄ | AlkLEᵥᵢᵢ | | |
| 1267 | E₁ₐE₅E₄ | AlkLEᵥᵢᵢ | | |
| 1268 | E_{1b}E₅E₄ | AlkLEᵥᵢᵢ | | |
| 1269 | E₁ₐEᵢE₄ | AlkLEᵥᵢᵢ | | |
| 1270 | E_{1b}EᵢE₄ | AlkLEᵥᵢᵢ | | |
| 1271 | E₁ₐEᵢᵢE₄ | AlkLEᵥᵢᵢ | | |
| 1272 | E_{1b}EᵢᵢE₄ | AlkLEᵥᵢᵢ | | |
| 1273 | E₁ₐEᵢᵢE_{iib}E₄ | AlkLEᵥᵢᵢ | | |
| 1274 | E_{1b}EᵢᵢE_{iib}E₄ | AlkLEᵥᵢᵢ | | |
| 1275 | E₁ₐE₅EᵢE₄ | AlkLEᵥᵢᵢ | | |
| 1276 | E_{1b}E₅EᵢE₄ | AlkLEᵥᵢᵢ | | |
| 1277 | E₁ₐE₅EᵢᵢE₄ | AlkLEᵥᵢᵢ | | |
| 1278 | E_{1b}E₅EᵢᵢE₄ | AlkLEᵥᵢᵢ | | |
| 1279 | E₁ₐE₅EᵢᵢE_{iib}E₄ | AlkLEᵥᵢᵢ | | |
| 1280 | E_{1b}E₅EᵢᵢE_{iib}E₄ | AlkLEᵥᵢᵢ | | |
| 1281 | E₁ₐEᵢᵢᵢ | AlkLEᵥᵢᵢ | | |
| 1282 | E_{1b}Eᵢᵢᵢ | AlkLEᵥᵢᵢ | | |
| 1283 | E₁ₐE₅Eᵢᵢᵢ | AlkLEᵥᵢᵢ | | |
| 1284 | E_{1b}E₅Eᵢᵢᵢ | AlkLEᵥᵢᵢ | | |
| 1285 | E₁ₐEᵢᵢᵢE₄ | AlkLEᵥᵢᵢ | | |
| 1286 | E_{1b}EᵢᵢᵢE₄ | AlkLEᵥᵢᵢ | | |
| 1287 | E₁ₐE₅EᵢᵢᵢE₄ | AlkLEᵥᵢᵢ | | |
| 1288 | E_{1b}E₅EᵢᵢᵢE₄ | AlkLEᵥᵢᵢ | | |
| 1289 | E₁ₐEᵢᵥ | AlkLEᵥᵢᵢ | | |
| 1290 | E_{1b}Eᵢᵥ | AlkLEᵥᵢᵢ | | |
| 1291 | E₁ₐE₅Eᵢᵥ | AlkLEᵥᵢᵢ | | |
| 1292 | E_{1b}E₅Eᵢᵥ | AlkLEᵥᵢᵢ | | |
| 1293 | E₁ₐEᵢᵥE₄ | AlkLEᵥᵢᵢ | | |
| 1294 | E_{1b}EᵢᵥE₄ | AlkLEᵥᵢᵢ | | |
| 1295 | E₁ₐE₅EᵢᵥE₄ | AlkLEᵥᵢᵢ | | |
| 1296 | E_{1b}E₅EᵢᵥE₄ | AlkLEᵥᵢᵢ | | |
| 1297 | E₁ₐ | AlkLEᵥᵢᵢ | E₄* | |
| 1298 | E_{1b} | AlkLEᵥᵢᵢ | E₄* | |
| 1299 | E₁ₐE₅ | AlkLEᵥᵢᵢ | E₄* | |
| 1300 | E_{1b}E₅ | AlkLEᵥᵢᵢ | E₄* | |
| 1301 | E₁ₐEᵢ | AlkLEᵥᵢᵢ | E₄* | |
| 1302 | E_{1b}Eᵢ | AlkLEᵥᵢᵢ | E₄* | |
| 1303 | E₁ₐEᵢᵢ | AlkLEᵥᵢᵢ | E₄* | |
| 1304 | E_{1b}Eᵢᵢ | AlkLEᵥᵢᵢ | E₄* | |
| 1305 | E₁ₐEᵢᵢE_{iib} | AlkLEᵥᵢᵢ | E₄* | |
| 1306 | E_{1b}EᵢᵢE_{iib} | AlkLEᵥᵢᵢ | E₄* | |
| 1307 | E₁ₐE₅Eᵢ | AlkLEᵥᵢᵢ | E₄* | |
| 1308 | E_{1b}E₅Eᵢ | AlkLEᵥᵢᵢ | E₄* | |
| 1309 | E₁ₐE₅Eᵢᵢ | AlkLEᵥᵢᵢ | E₄* | |
| 1310 | E_{1b}E₅Eᵢᵢ | AlkLEᵥᵢᵢ | E₄* | |
| 1311 | E₁ₐE₅EᵢᵢE_{iib} | AlkLEᵥᵢᵢ | E₄* | |
| 1312 | E_{1b}E₅EᵢᵢE_{iib} | AlkLEᵥᵢᵢ | E₄* | |
| 1313 | E₁ₐE₄ | AlkLEᵥᵢᵢ | E₄* | |
| 1314 | E_{1b}E₄ | AlkLEᵥᵢᵢ | E₄* | |
| 1315 | E₁ₐE₅E₄ | AlkLEᵥᵢᵢ | E₄* | |
| 1316 | E_{1b}E₅E₄ | AlkLEᵥᵢᵢ | E₄* | |
| 1317 | E₁ₐEᵢE₄ | AlkLEᵥᵢᵢ | E₄* | |
| 1318 | E_{1b}EᵢE₄ | AlkLEᵥᵢᵢ | E₄* | |
| 1319 | E₁ₐEᵢᵢE₄ | AlkLEᵥᵢᵢ | E₄* | |
| 1320 | E_{1b}EᵢᵢE₄ | AlkLEᵥᵢᵢ | E₄* | |
| 1321 | E₁ₐEᵢᵢE_{iib}E₄ | AlkLEᵥᵢᵢ | E₄* | |
| 1322 | E_{1b}EᵢᵢE_{iib}E₄ | AlkLEᵥᵢᵢ | E₄* | |
| 1323 | E₁ₐE₅EᵢE₄ | AlkLEᵥᵢᵢ | E₄* | |
| 1324 | E_{1b}E₅EᵢE₄ | AlkLEᵥᵢᵢ | E₄* | |
| 1325 | E₁ₐE₅EᵢᵢE₄ | AlkLEᵥᵢᵢ | E₄* | |
| 1326 | E_{1b}E₅EᵢᵢE₄ | AlkLEᵥᵢᵢ | E₄* | |
| 1327 | E₁ₐE₅EᵢᵢE_{iib}E₄ | AlkLEᵥᵢᵢ | E₄* | |
| 1328 | E_{1b}E₅EᵢᵢE_{iib}E₄ | AlkLEᵥᵢᵢ | E₄* | |
| 1329 | E₁ₐEᵢᵢᵢ | AlkLEᵥᵢᵢ | E₄* | |
| 1330 | E_{1b}Eᵢᵢᵢ | AlkLEᵥᵢᵢ | E₄* | |
| 1331 | E₁ₐE₅Eᵢᵢᵢ | AlkLEᵥᵢᵢ | E₄* | |
| 1332 | E_{1b}E₅Eᵢᵢᵢ | AlkLEᵥᵢᵢ | E₄* | |
| 1333 | E₁ₐEᵢᵢᵢE₄ | AlkLEᵥᵢᵢ | E₄* | |
| 1334 | E_{1b}EᵢᵢᵢE₄ | AlkLEᵥᵢᵢ | E₄* | |
| 1335 | E₁ₐE₅EᵢᵢᵢE₄ | AlkLEᵥᵢᵢ | E₄* | |
| 1336 | E_{1b}E₅EᵢᵢᵢE₄ | AlkLEᵥᵢᵢ | E₄* | |
| 1337 | E₁ₐEᵢᵥ | AlkLEᵥᵢᵢ | E₄* | |
| 1338 | E_{1b}Eᵢᵥ | AlkLEᵥᵢᵢ | E₄* | |
| 1339 | E₁ₐE₅Eᵢᵥ | AlkLEᵥᵢᵢ | E₄* | |
| 1340 | E_{1b}E₅Eᵢᵥ | AlkLEᵥᵢᵢ | E₄* | |
| 1341 | E₁ₐEᵢᵥE₄ | AlkLEᵥᵢᵢ | E₄* | |
| 1342 | E_{1b}EᵢᵥE₄ | AlkLEᵥᵢᵢ | E₄* | |
| 1343 | E₁ₐE₅EᵢᵥE₄ | AlkLEᵥᵢᵢ | E₄* | |
| 1344 | E_{1b}E₅EᵢᵥE₄ | AlkLEᵥᵢᵢ | E₄* | |
| 1345 | E₁ₐ | Eᵥᵢᵢ | | Eₐ |
| 1346 | E_{1b} | Eᵥᵢᵢ | | Eₐ |
| 1347 | E₁ₐE₅ | Eᵥᵢᵢ | | Eₐ |
| 1348 | E_{1b}E₅ | Eᵥᵢᵢ | | Eₐ |
| 1349 | E₁ₐEᵢ | Eᵥᵢᵢ | | Eₐ |
| 1350 | E_{1b}Eᵢ | Eᵥᵢᵢ | | Eₐ |
| 1351 | E₁ₐEᵢᵢ | Eᵥᵢᵢ | | Eₐ |
| 1352 | E_{1b}Eᵢᵢ | Eᵥᵢᵢ | | Eₐ |
| 1353 | E₁ₐEᵢᵢE_{iib} | Eᵥᵢᵢ | | Eₐ |
| 1354 | E_{1b}EᵢᵢE_{iib} | Eᵥᵢᵢ | | Eₐ |
| 1355 | E₁ₐE₅Eᵢ | Eᵥᵢᵢ | | Eₐ |
| 1356 | E_{1b}E₅Eᵢ | Eᵥᵢᵢ | | Eₐ |
| 1357 | E₁ₐE₅Eᵢᵢ | Eᵥᵢᵢ | | Eₐ |
| 1358 | E_{1b}E₅Eᵢᵢ | Eᵥᵢᵢ | | Eₐ |
| 1359 | E₁ₐE₅EᵢᵢE_{iib} | Eᵥᵢᵢ | | Eₐ |
| 1360 | E_{1b}E₅EᵢᵢE_{iib} | Eᵥᵢᵢ | | Eₐ |
| 1361 | E₁ₐE₄ | Eᵥᵢᵢ | | Eₐ |
| 1362 | E_{1b}E₄ | Eᵥᵢᵢ | | Eₐ |
| 1363 | E₁ₐE₅E₄ | Eᵥᵢᵢ | | Eₐ |
| 1364 | E_{1b}E₅E₄ | Eᵥᵢᵢ | | Eₐ |
| 1365 | E₁ₐEᵢE₄ | Eᵥᵢᵢ | | Eₐ |
| 1366 | E_{1b}EᵢE₄ | Eᵥᵢᵢ | | Eₐ |
| 1367 | E₁ₐEᵢᵢE₄ | Eᵥᵢᵢ | | Eₐ |
| 1368 | E_{1b}EᵢᵢE₄ | Eᵥᵢᵢ | | Eₐ |
| 1369 | E₁ₐEᵢᵢE_{iib}E₄ | Eᵥᵢᵢ | | Eₐ |
| 1370 | E_{1b}EᵢᵢE_{iib}E₄ | Eᵥᵢᵢ | | Eₐ |
| 1371 | E₁ₐE₅EᵢE₄ | Eᵥᵢᵢ | | Eₐ |
| 1372 | E_{1b}E₅EᵢE₄ | Eᵥᵢᵢ | | Eₐ |
| 1373 | E₁ₐE₅EᵢᵢE₄ | Eᵥᵢᵢ | | Eₐ |
| 1374 | E_{1b}E₅EᵢᵢE₄ | Eᵥᵢᵢ | | Eₐ |
| 1375 | E₁ₐE₅EᵢᵢE_{iib}E₄ | Eᵥᵢᵢ | | Eₐ |
| 1376 | E_{1b}E₅EᵢᵢE_{iib}E₄ | Eᵥᵢᵢ | | Eₐ |
| 1377 | E₁ₐEᵢᵢᵢ | Eᵥᵢᵢ | | Eₐ |
| 1378 | E_{1b}Eᵢᵢᵢ | Eᵥᵢᵢ | | Eₐ |
| 1379 | E₁ₐE₅Eᵢᵢᵢ | Eᵥᵢᵢ | | Eₐ |
| 1380 | E_{1b}E₅Eᵢᵢᵢ | Eᵥᵢᵢ | | Eₐ |
| 1381 | E₁ₐEᵢᵢᵢE₄ | Eᵥᵢᵢ | | Eₐ |
| 1382 | E_{1b}EᵢᵢᵢE₄ | Eᵥᵢᵢ | | Eₐ |
| 1383 | E₁ₐE₅EᵢᵢᵢE₄ | Eᵥᵢᵢ | | Eₐ |
| 1384 | E_{1b}E₅EᵢᵢᵢE₄ | Eᵥᵢᵢ | | Eₐ |
| 1385 | E₁ₐEᵢᵥ | Eᵥᵢᵢ | | Eₐ |
| 1386 | E_{1b}Eᵢᵥ | Eᵥᵢᵢ | | Eₐ |
| 1387 | E₁ₐE₅Eᵢᵥ | Eᵥᵢᵢ | | Eₐ |
| 1388 | E_{1b}E₅Eᵢᵥ | Eᵥᵢᵢ | | Eₐ |
| 1389 | E₁ₐEᵢᵥE₄ | Eᵥᵢᵢ | | Eₐ |
| 1390 | E_{1b}EᵢᵥE₄ | Eᵥᵢᵢ | | Eₐ |
| 1391 | E₁ₐE₅EᵢᵥE₄ | Eᵥᵢᵢ | | Eₐ |
| 1392 | E_{1b}E₅EᵢᵥE₄ | Eᵥᵢᵢ | | Eₐ |
| 1393 | E₁ₐ | Eᵥᵢᵢ | E₄* | Eₐ |
| 1394 | E_{1b} | Eᵥᵢᵢ | E₄* | Eₐ |
| 1395 | E₁ₐE₅ | Eᵥᵢᵢ | E₄* | Eₐ |
| 1396 | E_{1b}E₅ | Eᵥᵢᵢ | E₄* | Eₐ |
| 1397 | E₁ₐEᵢ | Eᵥᵢᵢ | E₄* | Eₐ |
| 1398 | E_{1b}Eᵢ | Eᵥᵢᵢ | E₄* | Eₐ |
| 1399 | E₁ₐEᵢᵢ | Eᵥᵢᵢ | E₄* | Eₐ |
| 1400 | E_{1b}Eᵢᵢ | Eᵥᵢᵢ | E₄* | Eₐ |
| 1401 | E₁ₐEᵢᵢE_{iib} | Eᵥᵢᵢ | E₄* | Eₐ |
| 1402 | E_{1b}EᵢᵢE_{iib} | Eᵥᵢᵢ | E₄* | Eₐ |
| 1403 | E₁ₐE₅Eᵢ | Eᵥᵢᵢ | E₄* | Eₐ |
| 1404 | E_{1b}E₅Eᵢ | Eᵥᵢᵢ | E₄* | Eₐ |
| 1405 | E₁ₐE₅Eᵢᵢ | Eᵥᵢᵢ | E₄* | Eₐ |
| 1406 | E_{1b}E₅Eᵢᵢ | Eᵥᵢᵢ | E₄* | Eₐ |
| 1407 | E₁ₐE₅EᵢᵢE_{iib} | Eᵥᵢᵢ | E₄* | Eₐ |
| 1408 | E_{1b}E₅EᵢᵢE_{iib} | Eᵥᵢᵢ | E₄* | Eₐ |
| 1409 | E₁ₐE₄ | Eᵥᵢᵢ | E₄* | Eₐ |
| 1410 | E_{1b}E₄ | Eᵥᵢᵢ | E₄* | Eₐ |
| 1411 | E₁ₐE₅E₄ | Eᵥᵢᵢ | E₄* | Eₐ |
| 1412 | E_{1b}E₅E₄ | Eᵥᵢᵢ | E₄* | Eₐ |
| 1413 | E₁ₐEᵢE₄ | Eᵥᵢᵢ | E₄* | Eₐ |
| 1414 | E_{1b}EᵢE₄ | Eᵥᵢᵢ | E₄* | Eₐ |
| 1415 | E₁ₐEᵢᵢE₄ | Eᵥᵢᵢ | E₄* | Eₐ |
| 1416 | E_{1b}EᵢᵢE₄ | Eᵥᵢᵢ | E₄* | Eₐ |
| 1417 | E₁ₐEᵢᵢE_{iib}E₄ | Eᵥᵢᵢ | E₄* | Eₐ |
| 1418 | E_{1b}EᵢᵢE_{iib}E₄ | Eᵥᵢᵢ | E₄* | Eₐ |
| 1419 | E₁ₐE₅EᵢE₄ | Eᵥᵢᵢ | E₄* | Eₐ |
| 1420 | E_{1b}E₅EᵢE₄ | Eᵥᵢᵢ | E₄* | Eₐ |
| 1421 | E₁ₐE₅EᵢᵢE₄ | Eᵥᵢᵢ | E₄* | Eₐ |
| 1422 | E_{1b}E₅EᵢᵢE₄ | Eᵥᵢᵢ | E₄* | Eₐ |
| 1423 | E₁ₐE₅EᵢᵢE_{iib}E₄ | Eᵥᵢᵢ | E₄* | Eₐ |
| 1424 | E_{1b}E₅EᵢᵢE_{iib}E₄ | Eᵥᵢᵢ | E₄* | Eₐ |
| 1425 | E₁ₐEᵢᵢᵢ | Eᵥᵢᵢ | E₄* | Eₐ |
| 1426 | E_{1b}Eᵢᵢᵢ | Eᵥᵢᵢ | E₄* | Eₐ |
| 1427 | E₁ₐE₅Eᵢᵢᵢ | Eᵥᵢᵢ | E₄* | Eₐ |
| 1428 | E_{1b}E₅Eᵢᵢᵢ | Eᵥᵢᵢ | E₄* | Eₐ |
| 1429 | E₁ₐEᵢᵢᵢE₄ | Eᵥᵢᵢ | E₄* | Eₐ |
| 1430 | E_{1b}EᵢᵢᵢE₄ | Eᵥᵢᵢ | E₄* | Eₐ |
| 1431 | E₁ₐE₅EᵢᵢᵢE₄ | Eᵥᵢᵢ | E₄* | Eₐ |
| 1432 | E_{1b}E₅EᵢᵢᵢE₄ | Eᵥᵢᵢ | E₄* | Eₐ |
| 1433 | E₁ₐEᵢᵥ | Eᵥᵢᵢ | E₄* | Eₐ |
| 1434 | E_{1b}Eᵢᵥ | Eᵥᵢᵢ | E₄* | Eₐ |
| 1435 | E₁ₐE₅Eᵢᵥ | Eᵥᵢᵢ | E₄* | Eₐ |
| 1436 | E_{1b}E₅Eᵢᵥ | Eᵥᵢᵢ | E₄* | Eₐ |
| 1437 | E₁ₐEᵢᵥE₄ | Eᵥᵢᵢ | E₄* | Eₐ |
| 1438 | E_{1b}EᵢᵥE₄ | Eᵥᵢᵢ | E₄* | Eₐ |
| 1439 | E₁ₐE₅EᵢᵥE₄ | Eᵥᵢᵢ | E₄* | Eₐ |
| 1440 | E_{1b}E₅EᵢᵥE₄ | Eᵥᵢᵢ | E₄* | Eₐ |
| 1441 | E₁ₐ | AlkLEᵥᵢᵢ | | Eₐ |
| 1442 | E_{1b} | AlkLEᵥᵢᵢ | | Eₐ |
| 1443 | E₁ₐE₅ | AlkLEᵥᵢᵢ | | Eₐ |
| 1444 | E_{1b}E₅ | AlkLEᵥᵢᵢ | | Eₐ |
| 1445 | E₁ₐEᵢ | AlkLEᵥᵢᵢ | | Eₐ |
| 1446 | E_{1b}Eᵢ | AlkLEᵥᵢᵢ | | Eₐ |
| 1447 | E₁ₐEᵢᵢ | AlkLEᵥᵢᵢ | | Eₐ |
| 1448 | E_{1b}Eᵢᵢ | AlkLEᵥᵢᵢ | | Eₐ |
| 1449 | E₁ₐEᵢᵢE_{iib} | AlkLEᵥᵢᵢ | | Eₐ |
| 1450 | E_{1b}EᵢᵢE_{iib} | AlkLEᵥᵢᵢ | | Eₐ |
| 1451 | E₁ₐE₅Eᵢ | AlkLEᵥᵢᵢ | | Eₐ |
| 1452 | E_{1b}E₅Eᵢ | AlkLEᵥᵢᵢ | | Eₐ |
| 1453 | E₁ₐE₅Eᵢᵢ | AlkLEᵥᵢᵢ | | Eₐ |
| 1454 | E_{1b}E₅Eᵢᵢ | AlkLEᵥᵢᵢ | | Eₐ |
| 1455 | E₁ₐE₅EᵢᵢE_{iib} | AlkLEᵥᵢᵢ | | Eₐ |
| 1456 | E_{1b}E₅EᵢᵢE_{iib} | AlkLEᵥᵢᵢ | | Eₐ |
| 1457 | E₁ₐE₄ | AlkLEᵥᵢᵢ | | Eₐ |
| 1458 | E_{1b}E₄ | AlkLEᵥᵢᵢ | | Eₐ |
| 1459 | E₁ₐE₅E₄ | AlkLEᵥᵢᵢ | | Eₐ |
| 1460 | E_{1b}E₅E₄ | AlkLEᵥᵢᵢ | | Eₐ |
| 1461 | E₁ₐEᵢE₄ | AlkLEᵥᵢᵢ | | Eₐ |
| 1462 | E_{1b}EᵢE₄ | AlkLEᵥᵢᵢ | | Eₐ |
| 1463 | E₁ₐEᵢᵢE₄ | AlkLEᵥᵢᵢ | | Eₐ |
| 1464 | E_{1b}EᵢᵢE₄ | AlkLEᵥᵢᵢ | | Eₐ |
| 1465 | E₁ₐEᵢᵢE_{iib}E₄ | AlkLEᵥᵢᵢ | | Eₐ |
| 1466 | E_{1b}EᵢᵢE_{iib}E₄ | AlkLEᵥᵢᵢ | | Eₐ |
| 1467 | E₁ₐE₅EᵢE₄ | AlkLEᵥᵢᵢ | | Eₐ |
| 1468 | E_{1b}E₅EᵢE₄ | AlkLEᵥᵢᵢ | | Eₐ |
| 1469 | E₁ₐE₅EᵢᵢE₄ | AlkLEᵥᵢᵢ | | Eₐ |
| 1470 | E_{1b}E₅EᵢᵢE₄ | AlkLEᵥᵢᵢ | | Eₐ |
| 1471 | E₁ₐE₅EᵢᵢE_{iib}E₄ | AlkLEᵥᵢᵢ | | Eₐ |
| 1472 | E_{1b}E₅EᵢᵢE_{iib}E₄ | AlkLEᵥᵢᵢ | | Eₐ |
| 1473 | E₁ₐEᵢᵢᵢ | AlkLEᵥᵢᵢ | | Eₐ |
| 1474 | E_{1b}Eᵢᵢᵢ | AlkLEᵥᵢᵢ | | Eₐ |
| 1475 | E₁ₐE₅Eᵢᵢᵢ | AlkLEᵥᵢᵢ | | Eₐ |
| 1476 | E_{1b}E₅Eᵢᵢᵢ | AlkLEᵥᵢᵢ | | Eₐ |
| 1477 | E₁ₐEᵢᵢᵢE₄ | AlkLEᵥᵢᵢ | | Eₐ |
| 1478 | E_{1b}EᵢᵢᵢE₄ | AlkLEᵥᵢᵢ | | Eₐ |
| 1479 | E₁ₐE₅EᵢᵢᵢE₄ | AlkLEᵥᵢᵢ | | Eₐ |
| 1480 | E_{1b}E₅EᵢᵢᵢE₄ | AlkLEᵥᵢᵢ | | Eₐ |
| 1481 | E₁ₐEᵢᵥ | AlkLEᵥᵢᵢ | | Eₐ |
| 1482 | E_{1b}Eᵢᵥ | AlkLEᵥᵢᵢ | | Eₐ |
| 1483 | E₁ₐE₅Eᵢᵥ | AlkLEᵥᵢᵢ | | Eₐ |
| 1484 | E_{1b}E₅Eᵢᵥ | AlkLEᵥᵢᵢ | | Eₐ |
| 1485 | E₁ₐEᵢᵥE₄ | AlkLEᵥᵢᵢ | | Eₐ |
| 1486 | E_{1b}EᵢᵥE₄ | AlkLEᵥᵢᵢ | | Eₐ |
| 1487 | E₁ₐE₅EᵢᵥE₄ | AlkLEᵥᵢᵢ | | Eₐ |
| 1488 | E_{1b}E₅EᵢᵥE₄ | AlkLEᵥᵢᵢ | | Eₐ |
| 1489 | E₁ₐ | AlkLEᵥᵢᵢ | E₄* | Eₐ |
| 1490 | E_{1b} | AlkLEᵥᵢᵢ | E₄* | Eₐ |
| 1491 | E₁ₐE₅ | AlkLEᵥᵢᵢ | E₄* | Eₐ |
| 1492 | E_{1b}E₅ | AlkLEᵥᵢᵢ | E₄* | Eₐ |
| 1493 | E₁ₐEᵢ | AlkLEᵥᵢᵢ | E₄* | Eₐ |
| 1494 | E_{1b}Eᵢ | AlkLEᵥᵢᵢ | E₄* | Eₐ |
| 1495 | E₁ₐEᵢᵢ | AlkLEᵥᵢᵢ | E₄* | Eₐ |
| 1496 | E_{1b}Eᵢᵢ | AlkLEᵥᵢᵢ | E₄* | Eₐ |
| 1497 | E₁ₐEᵢᵢE_{iib} | AlkLEᵥᵢᵢ | E₄* | Eₐ |
| 1498 | E_{1b}EᵢᵢE_{iib} | AlkLEᵥᵢᵢ | E₄* | Eₐ |
| 1499 | E₁ₐE₅Eᵢ | AlkLEᵥᵢᵢ | E₄* | Eₐ |
| 1500 | E_{1b}E₅Eᵢ | AlkLEᵥᵢᵢ | E₄* | Eₐ |
| 1501 | E₁ₐE₅Eᵢᵢ | AlkLEᵥᵢᵢ | E₄* | Eₐ |
| 1502 | E_{1b}E₅Eᵢᵢ | AlkLEᵥᵢᵢ | E₄* | Eₐ |
| 1503 | E₁ₐE₅EᵢᵢE_{iib} | AlkLEᵥᵢᵢ | E₄* | Eₐ |
| 1504 | E_{1b}E₅EᵢᵢE_{iib} | AlkLEᵥᵢᵢ | E₄* | Eₐ |
| 1505 | E₁ₐE₄ | AlkLEᵥᵢᵢ | E₄* | Eₐ |
| 1506 | E_{1b}E₄ | AlkLEᵥᵢᵢ | E₄* | Eₐ |
| 1507 | E₁ₐE₅E₄ | AlkLEᵥᵢᵢ | E₄* | Eₐ |
| 1508 | E_{1b}E₅E₄ | AlkLEᵥᵢᵢ | E₄* | Eₐ |
| 1509 | E₁ₐEᵢE₄ | AlkLEᵥᵢᵢ | E₄* | Eₐ |
| 1510 | E_{1b}EᵢE₄ | AlkLEᵥᵢᵢ | E₄* | Eₐ |
| 1511 | E₁ₐEᵢᵢE₄ | AlkLEᵥᵢᵢ | E₄* | Eₐ |
| 1512 | E_{1b}EᵢᵢE₄ | AlkLEᵥᵢᵢ | E₄* | Eₐ |
| 1513 | E₁ₐEᵢᵢE_{iib}E₄ | AlkLEᵥᵢᵢ | E₄* | Eₐ |
| 1514 | E_{1b}EᵢᵢE_{iib}E₄ | AlkLEᵥᵢᵢ | E₄* | Eₐ |
| 1515 | E₁ₐE₅EᵢE₄ | AlkLEᵥᵢᵢ | E₄* | Eₐ |
| 1516 | E_{1b}E₅EᵢE₄ | AlkLEᵥᵢᵢ | E₄* | Eₐ |
| 1517 | E₁ₐE₅EᵢᵢE₄ | AlkLEᵥᵢᵢ | E₄* | Eₐ |
| 1518 | E_{1b}E₅EᵢᵢE₄ | AlkLEᵥᵢᵢ | E₄* | Eₐ |
| 1519 | E₁ₐE₅EᵢᵢE_{iib}E₄ | AlkLEᵥᵢᵢ | E₄* | Eₐ |
| 1520 | E_{1b}E₅EᵢᵢE_{iib}E₄ | AlkLEᵥᵢᵢ | E₄* | Eₐ |
| 1521 | E₁ₐEᵢᵢᵢ | AlkLEᵥᵢᵢ | E₄* | Eₐ |
| 1522 | E_{1b}Eᵢᵢᵢ | AlkLEᵥᵢᵢ | E₄* | Eₐ |
| 1523 | E₁ₐE₅Eᵢᵢᵢ | AlkLEᵥᵢᵢ | E₄* | Eₐ |
| 1524 | E_{1b}E₅Eᵢᵢᵢ | AlkLEᵥᵢᵢ | E₄* | Eₐ |
| 1525 | E₁ₐEᵢᵢᵢE₄ | AlkLEᵥᵢᵢ | E₄* | Eₐ |
| 1526 | E_{1b}EᵢᵢᵢE₄ | AlkLEᵥᵢᵢ | E₄* | Eₐ |
| 1527 | E₁ₐE₅EᵢᵢᵢE₄ | AlkLEᵥᵢᵢ | E₄* | Eₐ |
| 1528 | E_{1b}E₅EᵢᵢᵢE₄ | AlkLEᵥᵢᵢ | E₄* | Eₐ |
| 1529 | E₁ₐEᵢᵥ | AlkLEᵥᵢᵢ | E₄* | Eₐ |
| 1530 | E_{1b}Eᵢᵥ | AlkLEᵥᵢᵢ | E₄* | Eₐ |
| 1531 | E₁ₐE₅Eᵢᵥ | AlkLEᵥᵢᵢ | E₄* | Eₐ |
| 1532 | E_{1b}E₅Eᵢᵥ | AlkLEᵥᵢᵢ | E₄* | Eₐ |
| 1533 | E₁ₐEᵢᵥE₄ | AlkLEᵥᵢᵢ | E₄* | Eₐ |
| 1534 | E_{1b}EᵢᵥE₄ | AlkLEᵥᵢᵢ | E₄* | Eₐ |
| 1535 | E₁ₐE₅EᵢᵥE₄ | AlkLEᵥᵢᵢ | E₄* | Eₐ |
| 1536 | E_{1b}E₅EᵢᵥE₄ | AlkLEᵥᵢᵢ | E₄* | Eₐ |
| 1537 | E₁ₐ | Eᵥᵢᵢ | | E_{b} |
| 1538 | E_{1b} | Eᵥᵢᵢ | | E_{b} |
| 1539 | E₁ₐE₅ | Eᵥᵢᵢ | | E_{b} |
| 1540 | E_{1b}E₅ | Eᵥᵢᵢ | | E_{b} |
| 1541 | E₁ₐEᵢ | Eᵥᵢᵢ | | E_{b} |
| 1542 | E_{1b}Eᵢ | Eᵥᵢᵢ | | E_{b} |
| 1543 | E₁ₐEᵢᵢ | Eᵥᵢᵢ | | E_{b} |
| 1544 | E_{1b}Eᵢᵢ | Eᵥᵢᵢ | | E_{b} |
| 1545 | E₁ₐEᵢᵢE_{iib} | Eᵥᵢᵢ | | E_{b} |
| 1546 | E_{1b}EᵢᵢE_{iib} | Eᵥᵢᵢ | | E_{b} |
| 1547 | E₁ₐE₅Eᵢ | Eᵥᵢᵢ | | E_{b} |
| 1548 | E_{1b}E₅Eᵢ | Eᵥᵢᵢ | | E_{b} |
| 1549 | E₁ₐE₅Eᵢᵢ | Eᵥᵢᵢ | | E_{b} |
| 1550 | E_{1b}E₅Eᵢᵢ | Eᵥᵢᵢ | | E_{b} |
| 1551 | E₁ₐE₅EᵢᵢE_{iib} | Eᵥᵢᵢ | | E_{b} |
| 1552 | E_{1b}E₅EᵢᵢE_{iib} | Eᵥᵢᵢ | | E_{b} |
| 1553 | E₁ₐE₄ | Eᵥᵢᵢ | | E_{b} |
| 1554 | E_{1b}E₄ | Eᵥᵢᵢ | | E_{b} |
| 1555 | E₁ₐE₅E₄ | Eᵥᵢᵢ | | E_{b} |
| 1556 | E_{1b}E₅E₄ | Eᵥᵢᵢ | | E_{b} |
| 1557 | E₁ₐEᵢE₄ | Eᵥᵢᵢ | | E_{b} |
| 1558 | E_{1b}EᵢE₄ | Eᵥᵢᵢ | | E_{b} |
| 1559 | E₁ₐEᵢᵢE₄ | Eᵥᵢᵢ | | E_{b} |
| 1560 | E_{1b}EᵢᵢE₄ | Eᵥᵢᵢ | | E_{b} |
| 1561 | E₁ₐEᵢᵢE_{iib}E₄ | Eᵥᵢᵢ | | E_{b} |
| 1562 | E_{1b}EᵢᵢE_{iib}E₄ | Eᵥᵢᵢ | | E_{b} |
| 1563 | E₁ₐE₅EᵢE₄ | Eᵥᵢᵢ | | E_{b} |
| 1564 | E_{1b}E₅EᵢE₄ | Eᵥᵢᵢ | | E_{b} |
| 1565 | E₁ₐE₅EᵢᵢE₄ | Eᵥᵢᵢ | | E_{b} |
| 1566 | E_{1b}E₅EᵢᵢE₄ | Eᵥᵢᵢ | | E_{b} |
| 1567 | E₁ₐE₅EᵢᵢE_{iib}E₄ | Eᵥᵢᵢ | | E_{b} |
| 1568 | E_{1b}E₅EᵢᵢE_{iib}E₄ | Eᵥᵢᵢ | | E_{b} |
| 1569 | E₁ₐEᵢᵢᵢ | Eᵥᵢᵢ | | E_{b} |
| 1570 | E_{1b}Eᵢᵢᵢ | Eᵥᵢᵢ | | E_{b} |
| 1571 | E₁ₐE₅Eᵢᵢᵢ | Eᵥᵢᵢ | | E_{b} |
| 1572 | E_{1b}E₅Eᵢᵢᵢ | Eᵥᵢᵢ | | E_{b} |
| 1573 | E₁ₐEᵢᵢᵢE₄ | Eᵥᵢᵢ | | E_{b} |
| 1574 | E_{1b}EᵢᵢᵢE₄ | Eᵥᵢᵢ | | E_{b} |
| 1575 | E₁ₐE₅EᵢᵢᵢE₄ | Eᵥᵢᵢ | | E_{b} |
| 1576 | E_{1b}E₅EᵢᵢᵢE₄ | Eᵥᵢᵢ | | E_{b} |
| 1577 | E₁ₐEᵢᵥ | Eᵥᵢᵢ | | E_{b} |
| 1578 | E_{1b}Eᵢᵥ | Eᵥᵢᵢ | | E_{b} |
| 1579 | E₁ₐE₅Eᵢᵥ | Eᵥᵢᵢ | | E_{b} |
| 1580 | E_{1b}E₅Eᵢᵥ | Eᵥᵢᵢ | | E_{b} |
| 1581 | E₁ₐEᵢᵥE₄ | Eᵥᵢᵢ | | E_{b} |
| 1582 | E_{1b}EᵢᵥE₄ | Eᵥᵢᵢ | | E_{b} |
| 1583 | E₁ₐE₅EᵢᵥE₄ | Eᵥᵢᵢ | | E_{b} |
| 1584 | E_{1b}E₅EᵢᵥE₄ | Eᵥᵢᵢ | | E_{b} |
| 1585 | E₁ₐ | Eᵥᵢᵢ | E₄* | E_{b} |
| 1586 | E_{1b} | Eᵥᵢᵢ | E₄* | E_{b} |
| 1587 | E₁ₐE₅ | Eᵥᵢᵢ | E₄* | E_{b} |
| 1588 | E_{1b}E₅ | Eᵥᵢᵢ | E₄* | E_{b} |
| 1589 | E₁ₐEᵢ | Eᵥᵢᵢ | E₄* | E_{b} |
| 1590 | E_{1b}Eᵢ | Eᵥᵢᵢ | E₄* | E_{b} |
| 1591 | E₁ₐEᵢᵢ | Eᵥᵢᵢ | E₄* | E_{b} |
| 1592 | E_{1b}Eᵢᵢ | Eᵥᵢᵢ | E₄* | E_{b} |
| 1593 | E₁ₐEᵢᵢE_{iib} | Eᵥᵢᵢ | E₄* | E_{b} |
| 1594 | E_{1b}EᵢᵢE_{iib} | Eᵥᵢᵢ | E₄* | E_{b} |
| 1595 | E₁ₐE₅Eᵢ | Eᵥᵢᵢ | E₄* | E_{b} |
| 1596 | E_{1b}E₅Eᵢ | Eᵥᵢᵢ | E₄* | E_{b} |
| 1597 | E₁ₐE₅Eᵢᵢ | Eᵥᵢᵢ | E₄* | E_{b} |
| 1598 | E_{1b}E₅Eᵢᵢ | Eᵥᵢᵢ | E₄* | E_{b} |
| 1599 | E₁ₐE₅EᵢᵢE_{iib} | Eᵥᵢᵢ | E₄* | E_{b} |
| 1600 | E_{1b}E₅EᵢᵢE_{iib} | Eᵥᵢᵢ | E₄* | E_{b} |
| 1601 | E₁ₐE₄ | Eᵥᵢᵢ | E₄* | E_{b} |
| 1602 | E_{1b}E₄ | Eᵥᵢᵢ | E₄* | E_{b} |
| 1603 | E₁ₐE₅E₄ | Eᵥᵢᵢ | E₄* | E_{b} |
| 1604 | E_{1b}E₅E₄ | Eᵥᵢᵢ | E₄* | E_{b} |
| 1605 | E₁ₐEᵢE₄ | Eᵥᵢᵢ | E₄* | E_{b} |
| 1606 | E_{1b}EᵢE₄ | Eᵥᵢᵢ | E₄* | E_{b} |
| 1607 | E₁ₐEᵢᵢE₄ | Eᵥᵢᵢ | E₄* | E_{b} |
| 1608 | E_{1b}EᵢᵢE₄ | Eᵥᵢᵢ | E₄* | E_{b} |
| 1609 | E₁ₐEᵢᵢE_{iib}E₄ | Eᵥᵢᵢ | E₄* | E_{b} |
| 1610 | E_{1b}EᵢᵢE_{iib}E₄ | Eᵥᵢᵢ | E₄* | E_{b} |
| 1611 | E₁ₐE₅EᵢE₄ | Eᵥᵢᵢ | E₄* | E_{b} |
| 1612 | E_{1b}E₅EᵢE₄ | Eᵥᵢᵢ | E₄* | E_{b} |
| 1613 | E₁ₐE₅EᵢᵢE₄ | Eᵥᵢᵢ | E₄* | E_{b} |
| 1614 | E_{1b}E₅EᵢᵢE₄ | Eᵥᵢᵢ | E₄* | E_{b} |
| 1615 | E₁ₐE₅EᵢᵢE_{iib}E₄ | Eᵥᵢᵢ | E₄* | E_{b} |
| 1616 | E_{1b}E₅EᵢᵢE_{iib}E₄ | Eᵥᵢᵢ | E₄* | E_{b} |
| 1617 | E₁ₐEᵢᵢᵢ | Eᵥᵢᵢ | E₄* | E_{b} |
| 1618 | E_{1b}Eᵢᵢᵢ | Eᵥᵢᵢ | E₄* | E_{b} |
| 1619 | E₁ₐE₅Eᵢᵢᵢ | Eᵥᵢᵢ | E₄* | E_{b} |
| 1620 | E_{1b}E₅Eᵢᵢᵢ | Eᵥᵢᵢ | E₄* | E_{b} |
| 1621 | E₁ₐEᵢᵢᵢE₄ | Eᵥᵢᵢ | E₄* | E_{b} |
| 1622 | E_{1b}EᵢᵢᵢE₄ | Eᵥᵢᵢ | E₄* | E_{b} |
| 1623 | E₁ₐE₅EᵢᵢᵢE₄ | Eᵥᵢᵢ | E₄* | E_{b} |
| 1624 | E_{1b}E₅EᵢᵢᵢE₄ | Eᵥᵢᵢ | E₄* | E_{b} |
| 1625 | E₁ₐEᵢᵥ | Eᵥᵢᵢ | E₄* | E_{b} |
| 1626 | E_{1b}Eᵢᵥ | Eᵥᵢᵢ | E₄* | E_{b} |
| 1627 | E₁ₐE₅Eᵢᵥ | Eᵥᵢᵢ | E₄* | E_{b} |
| 1628 | E_{1b}E₅Eᵢᵥ | Eᵥᵢᵢ | E₄* | E_{b} |
| 1629 | E₁ₐEᵢᵥE₄ | Eᵥᵢᵢ | E₄* | E_{b} |
| 1630 | E_{1b}EᵢᵥE₄ | Eᵥᵢᵢ | E₄* | E_{b} |
| 1631 | E₁ₐE₅EᵢᵥE₄ | Eᵥᵢᵢ | E₄* | E_{b} |
| 1632 | E_{1b}E₅EᵢᵥE₄ | Eᵥᵢᵢ | E₄* | E_{b} |
| 1633 | E₁ₐ | AlkLEᵥᵢᵢ | | E_{b} |
| 1634 | E_{1b} | AlkLEᵥᵢᵢ | | E_{b} |
| 1635 | E₁ₐE₅ | AlkLEᵥᵢᵢ | | E_{b} |
| 1636 | E_{1b}E₅ | AlkLEᵥᵢᵢ | | E_{b} |
| 1637 | E₁ₐEᵢ | AlkLEᵥᵢᵢ | | E_{b} |
| 1638 | E_{1b}Eᵢ | AlkLEᵥᵢᵢ | | E_{b} |
| 1639 | E₁ₐEᵢᵢ | AlkLEᵥᵢᵢ | | E_{b} |
| 1640 | E_{1b}Eᵢᵢ | AlkLEᵥᵢᵢ | | E_{b} |
| 1641 | E₁ₐEᵢᵢE_{iib} | AlkLEᵥᵢᵢ | | E_{b} |
| 1642 | E_{1b}EᵢᵢE_{iib} | AlkLEᵥᵢᵢ | | E_{b} |
| 1643 | E₁ₐE₅Eᵢ | AlkLEᵥᵢᵢ | | E_{b} |
| 1644 | E_{1b}E₅Eᵢ | AlkLEᵥᵢᵢ | | E_{b} |
| 1645 | E₁ₐE₅Eᵢᵢ | AlkLEᵥᵢᵢ | | E_{b} |
| 1646 | E_{1b}E₅Eᵢᵢ | AlkLEᵥᵢᵢ | | E_{b} |
| 1647 | E₁ₐE₅EᵢᵢE_{iib} | AlkLEᵥᵢᵢ | | E_{b} |
| 1648 | E_{1b}E₅EᵢᵢE_{iib} | AlkLEᵥᵢᵢ | | E_{b} |
| 1649 | E₁ₐE₄ | AlkLEᵥᵢᵢ | | E_{b} |
| 1650 | E_{1b}E₄ | AlkLEᵥᵢᵢ | | E_{b} |
| 1651 | E₁ₐE₅E₄ | AlkLEᵥᵢᵢ | | E_{b} |
| 1652 | E_{1b}E₅E₄ | AlkLEᵥᵢᵢ | | E_{b} |
| 1653 | E₁ₐEᵢE₄ | AlkLEᵥᵢᵢ | | E_{b} |
| 1654 | E_{1b}EᵢE₄ | AlkLEᵥᵢᵢ | | E_{b} |
| 1655 | E₁ₐEᵢᵢE₄ | AlkLEᵥᵢᵢ | | E_{b} |
| 1656 | E_{1b}EᵢᵢE₄ | AlkLEᵥᵢᵢ | | E_{b} |
| 1657 | E₁ₐEᵢᵢE_{iib}E₄ | AlkLEᵥᵢᵢ | | E_{b} |
| 1658 | E_{1b}EᵢᵢE_{iib}E₄ | AlkLEᵥᵢᵢ | | E_{b} |
| 1659 | E₁ₐE₅EᵢE₄ | AlkLEᵥᵢᵢ | | E_{b} |
| 1660 | E_{1b}E₅EᵢE₄ | AlkLEᵥᵢᵢ | | E_{b} |
| 1661 | E₁ₐE₅EᵢᵢE₄ | AlkLEᵥᵢᵢ | | E_{b} |
| 1662 | E_{1b}E₅EᵢᵢE₄ | AlkLEᵥᵢᵢ | | E_{b} |
| 1663 | E₁ₐE₅EᵢᵢE_{iib}E₄ | AlkLEᵥᵢᵢ | | E_{b} |
| 1664 | E_{1b}E₅EᵢᵢE_{iib}E₄ | AlkLEᵥᵢᵢ | | E_{b} |
| 1665 | E₁ₐEᵢᵢᵢ | AlkLEᵥᵢᵢ | | E_{b} |
| 1666 | E_{1b}Eᵢᵢᵢ | AlkLEᵥᵢᵢ | | E_{b} |
| 1667 | E₁ₐE₅Eᵢᵢᵢ | AlkLEᵥᵢᵢ | | E_{b} |
| 1668 | E_{1b}E₅Eᵢᵢᵢ | AlkLEᵥᵢᵢ | | E_{b} |
| 1669 | E₁ₐEᵢᵢᵢE₄ | AlkLEᵥᵢᵢ | | E_{b} |
| 1670 | E_{1b}EᵢᵢᵢE₄ | AlkLEᵥᵢᵢ | | E_{b} |
| 1671 | E₁ₐE₅EᵢᵢᵢE₄ | AlkLEᵥᵢᵢ | | E_{b} |
| 1672 | E_{1b}E₅EᵢᵢᵢE₄ | AlkLEᵥᵢᵢ | | E_{b} |
| 1673 | E₁ₐEᵢᵥ | AlkLEᵥᵢᵢ | | E_{b} |
| 1674 | E_{1b}Eᵢᵥ | AlkLEᵥᵢᵢ | | E_{b} |
| 1675 | E₁ₐE₅Eᵢᵥ | AlkLEᵥᵢᵢ | | E_{b} |
| 1676 | E_{1b}E₅Eᵢᵥ | AlkLEᵥᵢᵢ | | E_{b} |
| 1677 | E₁ₐEᵢᵥE₄ | AlkLEᵥᵢᵢ | | E_{b} |
| 1678 | E_{1b}EᵢᵥE₄ | AlkLEᵥᵢᵢ | | E_{b} |
| 1679 | E₁ₐE₅EᵢᵥE₄ | AlkLEᵥᵢᵢ | | E_{b} |
| 1680 | E_{1b}E₅EᵢᵥE₄ | AlkLEᵥᵢᵢ | | E_{b} |
| 1681 | E₁ₐ | AlkLEᵥᵢᵢ | E₄* | E_{b} |
| 1682 | E_{1b} | AlkLEᵥᵢᵢ | E₄* | E_{b} |
| 1683 | E₁ₐE₅ | AlkLEᵥᵢᵢ | E₄* | E_{b} |
| 1684 | E_{1b}E₅ | AlkLEᵥᵢᵢ | E₄* | E_{b} |
| 1685 | E₁ₐEᵢ | AlkLEᵥᵢᵢ | E₄* | E_{b} |
| 1686 | E_{1b}Eᵢ | AlkLEᵥᵢᵢ | E₄* | E_{b} |
| 1687 | E₁ₐEᵢᵢ | AlkLEᵥᵢᵢ | E₄* | E_{b} |
| 1688 | E_{1b}Eᵢᵢ | AlkLEᵥᵢᵢ | E₄* | E_{b} |
| 1689 | E₁ₐEᵢᵢE_{iib} | AlkLEᵥᵢᵢ | E₄* | E_{b} |
| 1690 | E_{1b}EᵢᵢE_{iib} | AlkLEᵥᵢᵢ | E₄* | E_{b} |
| 1691 | E₁ₐE₅Eᵢ | AlkLEᵥᵢᵢ | E₄* | E_{b} |
| 1692 | E_{1b}E₅Eᵢ | AlkLEᵥᵢᵢ | E₄* | E_{b} |
| 1693 | E₁ₐE₅Eᵢᵢ | AlkLEᵥᵢᵢ | E₄* | E_{b} |
| 1694 | E_{1b}E₅Eᵢᵢ | AlkLEᵥᵢᵢ | E₄* | E_{b} |
| 1695 | E₁ₐE₅EᵢᵢE_{iib} | AlkLEᵥᵢᵢ | E₄* | E_{b} |
| 1696 | E_{1b}E₅EᵢᵢE_{iib} | AlkLEᵥᵢᵢ | E₄* | E_{b} |
| 1697 | E₁ₐE₄ | AlkLEᵥᵢᵢ | E₄* | E_{b} |
| 1698 | E_{1b}E₄ | AlkLEᵥᵢᵢ | E₄* | E_{b} |
| 1699 | E₁ₐE₅E₄ | AlkLEᵥᵢᵢ | E₄* | E_{b} |
| 1700 | E_{1b}E₅E₄ | AlkLEᵥᵢᵢ | E₄* | E_{b} |
| 1701 | E₁ₐEᵢE₄ | AlkLEᵥᵢᵢ | E₄* | E_{b} |
| 1702 | E_{1b}EᵢE₄ | AlkLEᵥᵢᵢ | E₄* | E_{b} |
| 1703 | E₁ₐEᵢᵢE₄ | AlkLEᵥᵢᵢ | E₄* | E_{b} |
| 1704 | E_{1b}EᵢᵢE₄ | AlkLEᵥᵢᵢ | E₄* | E_{b} |
| 1705 | E₁ₐEᵢᵢE_{iib}E₄ | AlkLEᵥᵢᵢ | E₄* | E_{b} |
| 1706 | E_{1b}EᵢᵢE_{iib}E₄ | AlkLEᵥᵢᵢ | E₄* | E_{b} |
| 1707 | E₁ₐE₅EᵢE₄ | AlkLEᵥᵢᵢ | E₄* | E_{b} |
| 1708 | E_{1b}E₅EᵢE₄ | AlkLEᵥᵢᵢ | E₄* | E_{b} |
| 1709 | E₁ₐE₅EᵢᵢE₄ | AlkLEᵥᵢᵢ | E₄* | E_{b} |
| 1710 | E_{1b}E₅EᵢᵢE₄ | AlkLEᵥᵢᵢ | E₄* | E_{b} |
| 1711 | E₁ₐE₅EᵢᵢE_{iib}E₄ | AlkLEᵥᵢᵢ | E₄* | E_{b} |
| 1712 | E_{1b}E₅EᵢᵢE_{iib}E₄ | AlkLEᵥᵢᵢ | E₄* | E_{b} |
| 1713 | E₁ₐEᵢᵢᵢ | AlkLEᵥᵢᵢ | E₄* | E_{b} |
| 1714 | E_{1b}Eᵢᵢᵢ | AlkLEᵥᵢᵢ | E₄* | E_{b} |
| 1715 | E₁ₐE₅Eᵢᵢᵢ | AlkLEᵥᵢᵢ | E₄* | E_{b} |
| 1716 | E_{1b}E₅Eᵢᵢᵢ | AlkLEᵥᵢᵢ | E₄* | E_{b} |
| 1717 | E₁ₐEᵢᵢᵢE₄ | AlkLEᵥᵢᵢ | E₄* | E_{b} |
| 1718 | E_{1b}EᵢᵢᵢE₄ | AlkLEᵥᵢᵢ | E₄* | E_{b} |
| 1719 | E₁ₐE₅EᵢᵢᵢE₄ | AlkLEᵥᵢᵢ | E₄* | E_{b} |
| 1720 | E_{1b}E₅EᵢᵢᵢE₄ | AlkLEᵥᵢᵢ | E₄* | E_{b} |
| 1721 | E₁ₐEᵢᵥ | AlkLEᵥᵢᵢ | E₄* | E_{b} |
| 1722 | E_{1b}Eᵢᵥ | AlkLEᵥᵢᵢ | E₄* | E_{b} |
| 1723 | E₁ₐE₅Eᵢᵥ | AlkLEᵥᵢᵢ | E₄* | E_{b} |
| 1724 | E_{1b}E₅Eᵢᵥ | AlkLEᵥᵢᵢ | E₄* | E_{b} |
| 1725 | E₁ₐEᵢᵥE₄ | AlkLEᵥᵢᵢ | E₄* | E_{b} |
| 1726 | E_{1b}EᵢᵥE₄ | AlkLEᵥᵢᵢ | E₄* | E_{b} |
| 1727 | E₁ₐE₅EᵢᵥE₄ | AlkLEᵥᵢᵢ | E₄* | E_{b} |
| 1728 | E_{1b}E₅EᵢᵥE₄ | AlkLEᵥᵢᵢ | E₄* | E_{b} |
| 1729 | E₁ₐ | Eᵥᵢᵢ | | E_{d} |
| 1730 | E_{1b} | Eᵥᵢᵢ | | E_{d} |
| 1731 | E₁ₐE₅ | Eᵥᵢᵢ | | E_{d} |
| 1732 | E_{1b}E₅ | Eᵥᵢᵢ | | E_{d} |
| 1733 | E₁ₐEᵢ | Eᵥᵢᵢ | | E_{d} |
| 1734 | E_{1b}Eᵢ | Eᵥᵢᵢ | | E_{d} |
| 1735 | E₁ₐEᵢᵢ | Eᵥᵢᵢ | | E_{d} |
| 1736 | E_{1b}Eᵢᵢ | Eᵥᵢᵢ | | E_{d} |
| 1737 | E₁ₐEᵢᵢE_{iib} | Eᵥᵢᵢ | | E_{d} |
| 1738 | E_{1b}EᵢᵢE_{iib} | Eᵥᵢᵢ | | E_{d} |
| 1739 | E₁ₐE₅Eᵢ | Eᵥᵢᵢ | | E_{d} |
| 1740 | E_{1b}E₅Eᵢ | Eᵥᵢᵢ | | E_{d} |
| 1741 | E₁ₐE₅Eᵢᵢ | Eᵥᵢᵢ | | E_{d} |
| 1742 | E_{1b}E₅Eᵢᵢ | Eᵥᵢᵢ | | E_{d} |
| 1743 | E₁ₐE₅EᵢᵢE_{iib} | Eᵥᵢᵢ | | E_{d} |
| 1744 | E_{1b}E₅EᵢᵢE_{iib} | Eᵥᵢᵢ | | E_{d} |
| 1745 | E₁ₐE₄ | Eᵥᵢᵢ | | E_{d} |
| 1746 | E_{1b}E₄ | Eᵥᵢᵢ | | E_{d} |
| 1747 | E₁ₐE₅E₄ | Eᵥᵢᵢ | | E_{d} |
| 1748 | E_{1b}E₅E₄ | Eᵥᵢᵢ | | E_{d} |
| 1749 | E₁ₐEᵢE₄ | Eᵥᵢᵢ | | E_{d} |
| 1750 | E_{1b}EᵢE₄ | Eᵥᵢᵢ | | E_{d} |
| 1751 | E₁ₐEᵢᵢE₄ | Eᵥᵢᵢ | | E_{d} |
| 1752 | E_{1b}EᵢᵢE₄ | Eᵥᵢᵢ | | E_{d} |
| 1753 | E₁ₐEᵢᵢE_{iib}E₄ | Eᵥᵢᵢ | | E_{d} |
| 1754 | E_{1b}EᵢᵢE_{iib}E₄ | Eᵥᵢᵢ | | E_{d} |
| 1755 | E₁ₐE₅EᵢE₄ | Eᵥᵢᵢ | | E_{d} |
| 1756 | E_{1b}E₅EᵢE₄ | Eᵥᵢᵢ | | E_{d} |
| 1757 | E₁ₐE₅EᵢᵢE₄ | Eᵥᵢᵢ | | E_{d} |
| 1758 | E_{1b}E₅EᵢᵢE₄ | Eᵥᵢᵢ | | E_{d} |
| 1759 | E₁ₐE₅EᵢᵢE_{iib}E₄ | Eᵥᵢᵢ | | E_{d} |
| 1760 | E_{1b}E₅EᵢᵢE_{iib}E₄ | Eᵥᵢᵢ | | E_{d} |
| 1761 | E₁ₐEᵢᵢᵢ | Eᵥᵢᵢ | | E_{d} |
| 1762 | E_{1b}Eᵢᵢᵢ | Eᵥᵢᵢ | | E_{d} |
| 1763 | E₁ₐE₅Eᵢᵢᵢ | Eᵥᵢᵢ | | E_{d} |
| 1764 | E_{1b}E₅Eᵢᵢᵢ | Eᵥᵢᵢ | | E_{d} |
| 1765 | E₁ₐEᵢᵢᵢE₄ | Eᵥᵢᵢ | | E_{d} |
| 1766 | E_{1b}EᵢᵢᵢE₄ | Eᵥᵢᵢ | | E_{d} |
| 1767 | E₁ₐE₅EᵢᵢᵢE₄ | Eᵥᵢᵢ | | E_{d} |
| 1768 | E_{1b}E₅EᵢᵢᵢE₄ | Eᵥᵢᵢ | | E_{d} |
| 1769 | E₁ₐEᵢᵥ | Eᵥᵢᵢ | | E_{d} |
| 1770 | E_{1b}Eᵢᵥ | Eᵥᵢᵢ | | E_{d} |
| 1771 | E₁ₐE₅Eᵢᵥ | Eᵥᵢᵢ | | E_{d} |
| 1772 | E_{1b}E₅Eᵢᵥ | Eᵥᵢᵢ | | E_{d} |
| 1773 | E₁ₐEᵢᵥE₄ | Eᵥᵢᵢ | | E_{d} |
| 1774 | E_{1b}EᵢᵥE₄ | Eᵥᵢᵢ | | E_{d} |
| 1775 | E₁ₐE₅EᵢᵥE₄ | Eᵥᵢᵢ | | E_{d} |
| 1776 | E_{1b}E₅EᵢᵥE₄ | Eᵥᵢᵢ | | E_{d} |
| 1777 | E₁ₐ | Eᵥᵢᵢ | E₄* | E_{d} |
| 1778 | E_{1b} | Eᵥᵢᵢ | E₄* | E_{d} |
| 1779 | E₁ₐE₅ | Eᵥᵢᵢ | E₄* | E_{d} |
| 1780 | E_{1b}E₅ | Eᵥᵢᵢ | E₄* | E_{d} |
| 1781 | E₁ₐEᵢ | Eᵥᵢᵢ | E₄* | E_{d} |
| 1782 | E_{1b}Eᵢ | Eᵥᵢᵢ | E₄* | E_{d} |
| 1783 | E₁ₐEᵢᵢ | Eᵥᵢᵢ | E₄* | E_{d} |
| 1784 | E_{1b}Eᵢᵢ | Eᵥᵢᵢ | E₄* | E_{d} |
| 1785 | E₁ₐEᵢᵢE_{iib} | Eᵥᵢᵢ | E₄* | E_{d} |
| 1786 | E_{1b}EᵢᵢE_{iib} | Eᵥᵢᵢ | E₄* | E_{d} |
| 1787 | E₁ₐE₅Eᵢ | Eᵥᵢᵢ | E₄* | E_{d} |
| 1788 | E_{1b}E₅Eᵢ | Eᵥᵢᵢ | E₄* | E_{d} |
| 1789 | E₁ₐE₅Eᵢᵢ | Eᵥᵢᵢ | E₄* | E_{d} |
| 1790 | E_{1b}E₅Eᵢᵢ | Eᵥᵢᵢ | E₄* | E_{d} |
| 1791 | E₁ₐE₅EᵢᵢE_{iib} | Eᵥᵢᵢ | E₄* | E_{d} |
| 1792 | E_{1b}E₅EᵢᵢE_{iib} | Eᵥᵢᵢ | E₄* | E_{d} |
| 1793 | E₁ₐE₄ | Eᵥᵢᵢ | E₄* | E_{d} |
| 1794 | E_{1b}E₄ | Eᵥᵢᵢ | E₄* | E_{d} |
| 1795 | E₁ₐE₅E₄ | Eᵥᵢᵢ | E₄* | E_{d} |
| 1796 | E_{1b}E₅E₄ | Eᵥᵢᵢ | E₄* | E_{d} |
| 1797 | E₁ₐEᵢE₄ | Eᵥᵢᵢ | E₄* | E_{d} |
| 1798 | E_{1b}EᵢE₄ | Eᵥᵢᵢ | E₄* | E_{d} |
| 1799 | E₁ₐEᵢᵢE₄ | Eᵥᵢᵢ | E₄* | E_{d} |
| 1800 | E_{1b}EᵢᵢE₄ | Eᵥᵢᵢ | E₄* | E_{d} |
| 1801 | E₁ₐEᵢᵢE_{iib}E₄ | Eᵥᵢᵢ | E₄* | E_{d} |
| 1802 | E_{1b}EᵢᵢE_{iib}E₄ | Eᵥᵢᵢ | E₄* | E_{d} |
| 1803 | E₁ₐE₅EᵢE₄ | Eᵥᵢᵢ | E₄* | E_{d} |
| 1804 | E_{1b}E₅EᵢE₄ | Eᵥᵢᵢ | E₄* | E_{d} |
| 1805 | E₁ₐE₅EᵢᵢE₄ | Eᵥᵢᵢ | E₄* | E_{d} |
| 1806 | E_{1b}E₅EᵢᵢE₄ | Eᵥᵢᵢ | E₄* | E_{d} |
| 1807 | E₁ₐE₅EᵢᵢE_{iib}E₄ | Eᵥᵢᵢ | E₄* | E_{d} |
| 1808 | E_{1b}E₅EᵢᵢE_{iib}E₄ | Eᵥᵢᵢ | E₄* | E_{d} |
| 1809 | E₁ₐEᵢᵢᵢ | Eᵥᵢᵢ | E₄* | E_{d} |
| 1810 | E_{1b}Eᵢᵢᵢ | Eᵥᵢᵢ | E₄* | E_{d} |
| 1811 | E₁ₐE₅Eᵢᵢᵢ | Eᵥᵢᵢ | E₄* | E_{d} |
| 1812 | E_{1b}E₅Eᵢᵢᵢ | Eᵥᵢᵢ | E₄* | E_{d} |
| 1813 | E₁ₐEᵢᵢᵢE₄ | Eᵥᵢᵢ | E₄* | E_{d} |
| 1814 | E_{1b}EᵢᵢᵢE₄ | Eᵥᵢᵢ | E₄* | E_{d} |
| 1815 | E₁ₐE₅EᵢᵢᵢE₄ | Eᵥᵢᵢ | E₄* | E_{d} |
| 1816 | E_{1b}E₅EᵢᵢᵢE₄ | Eᵥᵢᵢ | E₄* | E_{d} |
| 1817 | E₁ₐEᵢᵥ | Eᵥᵢᵢ | E₄* | E_{d} |
| 1818 | E_{1b}Eᵢᵥ | Eᵥᵢᵢ | E₄* | E_{d} |
| 1819 | E₁ₐE₅Eᵢᵥ | Eᵥᵢᵢ | E₄* | E_{d} |
| 1820 | E_{1b}E₅Eᵢᵥ | Eᵥᵢᵢ | E₄* | E_{d} |
| 1821 | E₁ₐEᵢᵥE₄ | Eᵥᵢᵢ | E₄* | E_{d} |
| 1822 | E_{1b}EᵢᵥE₄ | Eᵥᵢᵢ | E₄* | E_{d} |
| 1823 | E₁ₐE₅EᵢᵥE₄ | Eᵥᵢᵢ | E₄* | E_{d} |
| 1824 | E_{1b}E₅EᵢᵥE₄ | Eᵥᵢᵢ | E₄* | E_{d} |
| 1825 | E₁ₐ | AlkLEᵥᵢᵢ | | E_{d} |
| 1826 | E_{1b} | AlkLEᵥᵢᵢ | | E_{d} |
| 1827 | E₁ₐE₅ | AlkLEᵥᵢᵢ | | E_{d} |
| 1828 | E_{1b}E₅ | AlkLEᵥᵢᵢ | | E_{d} |
| 1829 | E₁ₐEᵢ | AlkLEᵥᵢᵢ | | E_{d} |
| 1830 | E_{1b}Eᵢ | AlkLEᵥᵢᵢ | | E_{d} |
| 1831 | E₁ₐEᵢᵢ | AlkLEᵥᵢᵢ | | E_{d} |
| 1832 | E_{1b}Eᵢᵢ | AlkLEᵥᵢᵢ | | E_{d} |
| 1833 | E₁ₐEᵢᵢE_{iib} | AlkLEᵥᵢᵢ | | E_{d} |
| 1834 | E_{1b}EᵢᵢE_{iib} | AlkLEᵥᵢᵢ | | E_{d} |
| 1835 | E₁ₐE₅Eᵢ | AlkLEᵥᵢᵢ | | E_{d} |
| 1836 | E_{1b}E₅Eᵢ | AlkLEᵥᵢᵢ | | E_{d} |
| 1837 | E₁ₐE₅Eᵢᵢ | AlkLEᵥᵢᵢ | | E_{d} |
| 1838 | E_{1b}E₅Eᵢᵢ | AlkLEᵥᵢᵢ | | E_{d} |
| 1839 | E₁ₐE₅EᵢᵢE_{iib} | AlkLEᵥᵢᵢ | | E_{d} |
| 1840 | E_{1b}E₅EᵢᵢE_{iib} | AlkLEᵥᵢᵢ | | E_{d} |
| 1841 | E₁ₐE₄ | AlkLEᵥᵢᵢ | | E_{d} |
| 1842 | E_{1b}E₄ | AlkLEᵥᵢᵢ | | E_{d} |
| 1843 | E₁ₐE₅E₄ | AlkLEᵥᵢᵢ | | E_{d} |
| 1844 | E_{1b}E₅E₄ | AlkLEᵥᵢᵢ | | E_{d} |
| 1845 | E₁ₐEᵢE₄ | AlkLEᵥᵢᵢ | | E_{d} |
| 1846 | E_{1b}EᵢE₄ | AlkLEᵥᵢᵢ | | E_{d} |
| 1847 | E₁ₐEᵢᵢE₄ | AlkLEᵥᵢᵢ | | E_{d} |
| 1848 | E_{1b}EᵢᵢE₄ | AlkLEᵥᵢᵢ | | E_{d} |
| 1849 | E₁ₐEᵢᵢE_{iib}E₄ | AlkLEᵥᵢᵢ | | E_{d} |
| 1850 | E_{1b}EᵢᵢE_{iib}E₄ | AlkLEᵥᵢᵢ | | E_{d} |
| 1851 | E₁ₐE₅EᵢE₄ | AlkLEᵥᵢᵢ | | E_{d} |
| 1852 | E_{1b}E₅EᵢE₄ | AlkLEᵥᵢᵢ | | E_{d} |
| 1853 | E₁ₐE₅EᵢᵢE₄ | AlkLEᵥᵢᵢ | | E_{d} |
| 1854 | E_{1b}E₅EᵢᵢE₄ | AlkLEᵥᵢᵢ | | E_{d} |
| 1855 | E₁ₐE₅EᵢᵢE_{iib}E₄ | AlkLEᵥᵢᵢ | | E_{d} |
| 1856 | E_{1b}E₅EᵢᵢE_{iib}E₄ | AlkLEᵥᵢᵢ | | E_{d} |
| 1857 | E₁ₐEᵢᵢᵢ | AlkLEᵥᵢᵢ | | E_{d} |
| 1858 | E_{1b}Eᵢᵢᵢ | AlkLEᵥᵢᵢ | | E_{d} |
| 1859 | E₁ₐE₅Eᵢᵢᵢ | AlkLEᵥᵢᵢ | | E_{d} |
| 1860 | E_{1b}E₅Eᵢᵢᵢ | AlkLEᵥᵢᵢ | | E_{d} |
| 1861 | E₁ₐEᵢᵢᵢE₄ | AlkLEᵥᵢᵢ | | E_{d} |
| 1862 | E_{1b}EᵢᵢᵢE₄ | AlkLEᵥᵢᵢ | | E_{d} |
| 1863 | E₁ₐE₅EᵢᵢᵢE₄ | AlkLEᵥᵢᵢ | | E_{d} |
| 1864 | E_{1b}E₅EᵢᵢᵢE₄ | AlkLEᵥᵢᵢ | | E_{d} |
| 1865 | E₁ₐEᵢᵥ | AlkLEᵥᵢᵢ | | E_{d} |
| 1866 | E_{1b}Eᵢᵥ | AlkLEᵥᵢᵢ | | E_{d} |
| 1867 | E₁ₐE₅Eᵢᵥ | AlkLEᵥᵢᵢ | | E_{d} |
| 1868 | E_{1b}E₅Eᵢᵥ | AlkLEᵥᵢᵢ | | E_{d} |
| 1869 | E₁ₐEᵢᵥE₄ | AlkLEᵥᵢᵢ | | E_{d} |
| 1870 | E_{1b}EᵢᵥE₄ | AlkLEᵥᵢᵢ | | E_{d} |
| 1871 | E₁ₐE₅EᵢᵥE₄ | AlkLEᵥᵢᵢ | | E_{d} |
| 1872 | E_{1b}E₅EᵢᵥE₄ | AlkLEᵥᵢᵢ | | E_{d} |
| 1873 | E₁ₐ | AlkLEᵥᵢᵢ | E₄* | E_{d} |
| 1874 | E_{1b} | AlkLEᵥᵢᵢ | E₄* | E_{d} |
| 1875 | E₁ₐE₅ | AlkLEᵥᵢᵢ | E₄* | E_{d} |
| 1876 | E_{1b}E₅ | AlkLEᵥᵢᵢ | E₄* | E_{d} |
| 1877 | E₁ₐEᵢ | AlkLEᵥᵢᵢ | E₄* | E_{d} |
| 1878 | E_{1b}Eᵢ | AlkLEᵥᵢᵢ | E₄* | E_{d} |
| 1879 | E₁ₐEᵢᵢ | AlkLEᵥᵢᵢ | E₄* | E_{d} |
| 1880 | E_{1b}Eᵢᵢ | AlkLEᵥᵢᵢ | E₄* | E_{d} |
| 1881 | E₁ₐEᵢᵢE_{iib} | AlkLEᵥᵢᵢ | E₄* | E_{d} |
| 1882 | E_{1b}EᵢᵢE_{iib} | AlkLEᵥᵢᵢ | E₄* | E_{d} |
| 1883 | E₁ₐE₅Eᵢ | AlkLEᵥᵢᵢ | E₄* | E_{d} |
| 1884 | E_{1b}E₅Eᵢ | AlkLEᵥᵢᵢ | E₄* | E_{d} |
| 1885 | E₁ₐE₅Eᵢᵢ | AlkLEᵥᵢᵢ | E₄* | E_{d} |
| 1886 | E_{1b}E₅Eᵢᵢ | AlkLEᵥᵢᵢ | E₄* | E_{d} |
| 1887 | E₁ₐE₅EᵢᵢE_{iib} | AlkLEᵥᵢᵢ | E₄* | E_{d} |
| 1888 | E_{1b}E₅EᵢᵢE_{iib} | AlkLEᵥᵢᵢ | E₄* | E_{d} |
| 1889 | E₁ₐE₄ | AlkLEᵥᵢᵢ | E₄* | E_{d} |
| 1890 | E_{1b}E₄ | AlkLEᵥᵢᵢ | E₄* | E_{d} |
| 1891 | E₁ₐE₅E₄ | AlkLEᵥᵢᵢ | E₄* | E_{d} |
| 1892 | E_{1b}E₅E₄ | AlkLEᵥᵢᵢ | E₄* | E_{d} |
| 1893 | E₁ₐEᵢE₄ | AlkLEᵥᵢᵢ | E₄* | E_{d} |
| 1894 | E_{1b}EᵢE₄ | AlkLEᵥᵢᵢ | E₄* | E_{d} |
| 1895 | E₁ₐEᵢᵢE₄ | AlkLEᵥᵢᵢ | E₄* | E_{d} |
| 1896 | E_{1b}EᵢᵢE₄ | AlkLEᵥᵢᵢ | E₄* | E_{d} |
| 1897 | E₁ₐEᵢᵢE_{iib}E₄ | AlkLEᵥᵢᵢ | E₄* | E_{d} |
| 1898 | E_{1b}EᵢᵢE_{iib}E₄ | AlkLEᵥᵢᵢ | E₄* | E_{d} |
| 1899 | E₁ₐE₅EᵢE₄ | AlkLEᵥᵢᵢ | E₄* | E_{d} |
| 1900 | E_{1b}E₅EᵢE₄ | AlkLEᵥᵢᵢ | E₄* | E_{d} |
| 1901 | E₁ₐE₅EᵢᵢE₄ | AlkLEᵥᵢᵢ | E₄* | E_{d} |
| 1902 | E_{1b}E₅EᵢᵢE₄ | AlkLEᵥᵢᵢ | E₄* | E_{d} |
| 1903 | E₁ₐE₅EᵢᵢE_{iib}E₄ | AlkLEᵥᵢᵢ | E₄* | E_{d} |
| 1904 | E_{1b}E₅EᵢᵢE_{iib}E₄ | AlkLEᵥᵢᵢ | E₄* | E_{d} |
| 1905 | E₁ₐEᵢᵢᵢ | AlkLEᵥᵢᵢ | E₄* | E_{d} |
| 1906 | E_{1b}Eᵢᵢᵢ | AlkLEᵥᵢᵢ | E₄* | E_{d} |
| 1907 | E₁ₐE₅Eᵢᵢᵢ | AlkLEᵥᵢᵢ | E₄* | E_{d} |
| 1908 | E_{1b}E₅Eᵢᵢᵢ | AlkLEᵥᵢᵢ | E₄* | E_{d} |
| 1909 | E₁ₐEᵢᵢᵢE₄ | AlkLEᵥᵢᵢ | E₄* | E_{d} |
| 1910 | E_{1b}EᵢᵢᵢE₄ | AlkLEᵥᵢᵢ | E₄* | E_{d} |
| 1911 | E₁ₐE₅EᵢᵢᵢE₄ | AlkLEᵥᵢᵢ | E₄* | E_{d} |
| 1912 | E_{1b}E₅EᵢᵢᵢE₄ | AlkLEᵥᵢᵢ | E₄* | E_{d} |
| 1913 | E₁ₐEᵢᵥ | AlkLEᵥᵢᵢ | E₄* | E_{d} |
| 1914 | E_{1b}Eᵢᵥ | AlkLEᵥᵢᵢ | E₄* | E_{d} |
| 1915 | E₁ₐE₅Eᵢᵥ | AlkLEᵥᵢᵢ | E₄* | E_{d} |
| 1916 | E_{1b}E₅Eᵢᵥ | AlkLEᵥᵢᵢ | E₄* | E_{d} |
| 1917 | E₁ₐEᵢᵥE₄ | AlkLEᵥᵢᵢ | E₄* | E_{d} |
| 1918 | E_{1b}EᵢᵥE₄ | AlkLEᵥᵢᵢ | E₄* | E_{d} |
| 1919 | E₁ₐE₅EᵢᵥE₄ | AlkLEᵥᵢᵢ | E₄* | E_{d} |
| 1920 | E_{1b}E₅EᵢᵥE₄ | AlkLEᵥᵢᵢ | E₄* | E_{d} |
| 1921 | E₁ₐ | Eᵥᵢᵢ | | Eₑ |
| 1922 | E_{1b} | Eᵥᵢᵢ | | Eₑ |
| 1923 | E₁ₐE₅ | Eᵥᵢᵢ | | Eₑ |
| 1924 | E_{1b}E₅ | Eᵥᵢᵢ | | Eₑ |
| 1925 | E₁ₐEᵢ | Eᵥᵢᵢ | | Eₑ |
| 1926 | E_{1b}Eᵢ | Eᵥᵢᵢ | | Eₑ |
| 1927 | E₁ₐEᵢᵢ | Eᵥᵢᵢ | | Eₑ |
| 1928 | E_{1b}Eᵢᵢ | Eᵥᵢᵢ | | Eₑ |
| 1929 | E₁ₐEᵢᵢE_{iib} | Eᵥᵢᵢ | | Eₑ |
| 1930 | E_{1b}EᵢᵢE_{iib} | Eᵥᵢᵢ | | Eₑ |
| 1931 | E₁ₐE₅Eᵢ | Eᵥᵢᵢ | | Eₑ |
| 1932 | E_{1b}E₅Eᵢ | Eᵥᵢᵢ | | Eₑ |
| 1933 | E₁ₐE₅Eᵢᵢ | Eᵥᵢᵢ | | Eₑ |
| 1934 | E_{1b}E₅Eᵢᵢ | Eᵥᵢᵢ | | Eₑ |
| 1935 | E₁ₐE₅EᵢᵢE_{iib} | Eᵥᵢᵢ | | Eₑ |
| 1936 | E_{1b}E₅EᵢᵢE_{iib} | Eᵥᵢᵢ | | Eₑ |
| 1937 | E₁ₐE₄ | Eᵥᵢᵢ | | Eₑ |
| 1938 | E_{1b}E₄ | Eᵥᵢᵢ | | Eₑ |
| 1939 | E₁ₐE₅E₄ | Eᵥᵢᵢ | | Eₑ |
| 1940 | E_{1b}E₅E₄ | Eᵥᵢᵢ | | Eₑ |
| 1941 | E₁ₐEᵢE₄ | Eᵥᵢᵢ | | Eₑ |
| 1942 | E_{1b}EᵢE₄ | Eᵥᵢᵢ | | Eₑ |
| 1943 | E₁ₐEᵢᵢE₄ | Eᵥᵢᵢ | | Eₑ |
| 1944 | E_{1b}EᵢᵢE₄ | Eᵥᵢᵢ | | Eₑ |
| 1945 | E₁ₐEᵢᵢE_{iib}E₄ | Eᵥᵢᵢ | | Eₑ |
| 1946 | E_{1b}EᵢᵢE_{iib}E₄ | Eᵥᵢᵢ | | Eₑ |
| 1947 | E₁ₐE₅EᵢE₄ | Eᵥᵢᵢ | | Eₑ |
| 1948 | E_{1b}E₅EᵢE₄ | Eᵥᵢᵢ | | Eₑ |
| 1949 | E₁ₐE₅EᵢᵢE₄ | Eᵥᵢᵢ | | Eₑ |
| 1950 | E_{1b}E₅EᵢᵢE₄ | Eᵥᵢᵢ | | Eₑ |
| 1951 | E₁ₐE₅EᵢᵢE_{iib}E₄ | Eᵥᵢᵢ | | Eₑ |
| 1952 | E_{1b}E₅EᵢᵢE_{iib}E₄ | Eᵥᵢᵢ | | Eₑ |
| 1953 | E₁ₐEᵢᵢᵢ | Eᵥᵢᵢ | | Eₑ |
| 1954 | E_{1b}Eᵢᵢᵢ | Eᵥᵢᵢ | | Eₑ |
| 1955 | E₁ₐE₅Eᵢᵢᵢ | Eᵥᵢᵢ | | Eₑ |
| 1956 | E_{1b}E₅Eᵢᵢᵢ | Eᵥᵢᵢ | | Eₑ |
| 1957 | E₁ₐEᵢᵢᵢE₄ | Eᵥᵢᵢ | | Eₑ |
| 1958 | E_{1b}EᵢᵢᵢE₄ | Eᵥᵢᵢ | | Eₑ |
| 1959 | E₁ₐE₅EᵢᵢᵢE₄ | Eᵥᵢᵢ | | Eₑ |
| 1960 | E_{1b}E₅EᵢᵢᵢE₄ | Eᵥᵢᵢ | | Eₑ |
| 1961 | E₁ₐEᵢᵥ | Eᵥᵢᵢ | | Eₑ |
| 1962 | E_{1b}Eᵢᵥ | Eᵥᵢᵢ | | Eₑ |
| 1963 | E₁ₐE₅Eᵢᵥ | Eᵥᵢᵢ | | Eₑ |
| 1964 | E_{1b}E₅Eᵢᵥ | Eᵥᵢᵢ | | Eₑ |
| 1965 | E₁ₐEᵢᵥE₄ | Eᵥᵢᵢ | | Eₑ |
| 1966 | E_{1b}EᵢᵥE₄ | Eᵥᵢᵢ | | Eₑ |
| 1967 | E₁ₐE₅EᵢᵥE₄ | Eᵥᵢᵢ | | Eₑ |
| 1968 | E_{1b}E₅EᵢᵥE₄ | Eᵥᵢᵢ | | Eₑ |
| 1969 | E₁ₐ | Eᵥᵢᵢ | E₄* | Eₑ |
| 1970 | E_{1b} | Eᵥᵢᵢ | E₄* | Eₑ |
| 1971 | E₁ₐE₅ | Eᵥᵢᵢ | E₄* | Eₑ |
| 1972 | E_{1b}E₅ | Eᵥᵢᵢ | E₄* | Eₑ |
| 1973 | E₁ₐEᵢ | Eᵥᵢᵢ | E₄* | Eₑ |
| 1974 | E_{1b}Eᵢ | Eᵥᵢᵢ | E₄* | Eₑ |
| 1975 | E₁ₐEᵢᵢ | Eᵥᵢᵢ | E₄* | Eₑ |
| 1976 | E_{1b}Eᵢᵢ | Eᵥᵢᵢ | E₄* | Eₑ |
| 1977 | E₁ₐEᵢᵢE_{iib} | Eᵥᵢᵢ | E₄* | Eₑ |
| 1978 | E_{1b}EᵢᵢE_{iib} | Eᵥᵢᵢ | E₄* | Eₑ |
| 1979 | E₁ₐE₅Eᵢ | Eᵥᵢᵢ | E₄* | Eₑ |
| 1980 | E_{1b}E₅Eᵢ | Eᵥᵢᵢ | E₄* | Eₑ |
| 1981 | E₁ₐE₅Eᵢᵢ | Eᵥᵢᵢ | E₄* | Eₑ |
| 1982 | E_{1b}E₅Eᵢᵢ | Eᵥᵢᵢ | E₄* | Eₑ |
| 1983 | E₁ₐE₅EᵢᵢE_{iib} | Eᵥᵢᵢ | E₄* | Eₑ |
| 1984 | E_{1b}E₅EᵢᵢE_{iib} | Eᵥᵢᵢ | E₄* | Eₑ |
| 1985 | E₁ₐE₄ | Eᵥᵢᵢ | E₄* | Eₑ |
| 1986 | E_{1b}E₄ | Eᵥᵢᵢ | E₄* | Eₑ |
| 1987 | E₁ₐE₅E₄ | Eᵥᵢᵢ | E₄* | Eₑ |
| 1988 | E_{1b}E₅E₄ | Eᵥᵢᵢ | E₄* | Eₑ |
| 1989 | E₁ₐEᵢE₄ | Eᵥᵢᵢ | E₄* | Eₑ |
| 1990 | E_{1b}EᵢE₄ | Eᵥᵢᵢ | E₄* | Eₑ |
| 1991 | E₁ₐEᵢᵢE₄ | Eᵥᵢᵢ | E₄* | Eₑ |
| 1992 | E_{1b}EᵢᵢE₄ | Eᵥᵢᵢ | E₄* | Eₑ |
| 1993 | E₁ₐEᵢᵢE_{iib}E₄ | Eᵥᵢᵢ | E₄* | Eₑ |
| 1994 | E_{1b}EᵢᵢE_{iib}E₄ | Eᵥᵢᵢ | E₄* | Eₑ |
| 1995 | E₁ₐE₅EᵢE₄ | Eᵥᵢᵢ | E₄* | Eₑ |
| 1996 | E_{1b}E₅EᵢE₄ | Eᵥᵢᵢ | E₄* | Eₑ |
| 1997 | E₁ₐE₅EᵢᵢE₄ | Eᵥᵢᵢ | E₄* | Eₑ |
| 1998 | E_{1b}E₅EᵢᵢE₄ | Eᵥᵢᵢ | E₄* | Eₑ |
| 1999 | E₁ₐE₅EᵢᵢE_{iib}E₄ | Eᵥᵢᵢ | E₄* | Eₑ |
| 2000 | E_{1b}E₅EᵢᵢE_{iib}E₄ | Eᵥᵢᵢ | E₄* | Eₑ |
| 2001 | E₁ₐEᵢᵢᵢ | Eᵥᵢᵢ | E₄* | Eₑ |
| 2002 | E_{1b}Eᵢᵢᵢ | Eᵥᵢᵢ | E₄* | Eₑ |
| 2003 | E₁ₐE₅Eᵢᵢᵢ | Eᵥᵢᵢ | E₄* | Eₑ |
| 2004 | E_{1b}E₅Eᵢᵢᵢ | Eᵥᵢᵢ | E₄* | Eₑ |
| 2005 | E₁ₐEᵢᵢᵢE₄ | Eᵥᵢᵢ | E₄* | Eₑ |
| 2006 | E_{1b}EᵢᵢᵢE₄ | Eᵥᵢᵢ | E₄* | Eₑ |
| 2007 | E₁ₐE₅EᵢᵢᵢE₄ | Eᵥᵢᵢ | E₄* | Eₑ |
| 2008 | E_{1b}E₅EᵢᵢᵢE₄ | Eᵥᵢᵢ | E₄* | Eₑ |
| 2009 | E₁ₐEᵢᵥ | Eᵥᵢᵢ | E₄* | Eₑ |
| 2010 | E_{1b}Eᵢᵥ | Eᵥᵢᵢ | E₄* | Eₑ |
| 2011 | E₁ₐE₅Eᵢᵥ | Eᵥᵢᵢ | E₄* | Eₑ |
| 2012 | E_{1b}E₅Eᵢᵥ | Eᵥᵢᵢ | E₄* | Eₑ |
| 2013 | E₁ₐEᵢᵥE₄ | Eᵥᵢᵢ | E₄* | Eₑ |
| 2014 | E_{1b}EᵢᵥE₄ | Eᵥᵢᵢ | E₄* | Eₑ |
| 2015 | E₁ₐE₅EᵢᵥE₄ | Eᵥᵢᵢ | E₄* | Eₑ |
| 2016 | E_{1b}E₅EᵢᵥE₄ | Eᵥᵢᵢ | E₄* | Eₑ |
| 2017 | E₁ₐ | AlkLEᵥᵢᵢ | | Eₑ |
| 2018 | E_{1b} | AlkLEᵥᵢᵢ | | Eₑ |
| 2019 | E₁ₐE₅ | AlkLEᵥᵢᵢ | | Eₑ |
| 2020 | E_{1b}E₅ | AlkLEᵥᵢᵢ | | Eₑ |
| 2021 | E₁ₐEᵢ | AlkLEᵥᵢᵢ | | Eₑ |
| 2022 | E_{1b}Eᵢ | AlkLEᵥᵢᵢ | | Eₑ |
| 2023 | E₁ₐEᵢᵢ | AlkLEᵥᵢᵢ | | Eₑ |
| 2024 | E_{1b}Eᵢᵢ | AlkLEᵥᵢᵢ | | Eₑ |
| 2025 | E₁ₐEᵢᵢE_{iib} | AlkLEᵥᵢᵢ | | Eₑ |
| 2026 | E_{1b}EᵢᵢE_{iib} | AlkLEᵥᵢᵢ | | Eₑ |
| 2027 | E₁ₐE₅Eᵢ | AlkLEᵥᵢᵢ | | Eₑ |
| 2028 | E_{1b}E₅Eᵢ | AlkLEᵥᵢᵢ | | Eₑ |
| 2029 | E₁ₐE₅Eᵢᵢ | AlkLEᵥᵢᵢ | | Eₑ |
| 2030 | E_{1b}E₅Eᵢᵢ | AlkLEᵥᵢᵢ | | Eₑ |
| 2031 | E₁ₐE₅EᵢᵢE_{iib} | AlkLEᵥᵢᵢ | | Eₑ |
| 2032 | E_{1b}E₅EᵢᵢE_{iib} | AlkLEᵥᵢᵢ | | Eₑ |
| 2033 | E₁ₐE₄ | AlkLEᵥᵢᵢ | | Eₑ |
| 2034 | E_{1b}E₄ | AlkLEᵥᵢᵢ | | Eₑ |
| 2035 | E₁ₐE₅E₄ | AlkLEᵥᵢᵢ | | Eₑ |
| 2036 | E_{1b}E₅E₄ | AlkLEᵥᵢᵢ | | Eₑ |
| 2037 | E₁ₐEᵢE₄ | AlkLEᵥᵢᵢ | | Eₑ |
| 2038 | E_{1b}EᵢE₄ | AlkLEᵥᵢᵢ | | Eₑ |
| 2039 | E₁ₐEᵢᵢE₄ | AlkLEᵥᵢᵢ | | Eₑ |
| 2040 | E_{1b}EᵢᵢE₄ | AlkLEᵥᵢᵢ | | Eₑ |
| 2041 | E₁ₐEᵢᵢE_{iib}E₄ | AlkLEᵥᵢᵢ | | Eₑ |
| 2042 | E_{1b}EᵢᵢE_{iib}E₄ | AlkLEᵥᵢᵢ | | Eₑ |
| 2043 | E₁ₐE₅EᵢE₄ | AlkLEᵥᵢᵢ | | Eₑ |
| 2044 | E_{1b}E₅EᵢE₄ | AlkLEᵥᵢᵢ | | Eₑ |
| 2045 | E₁ₐE₅EᵢᵢE₄ | AlkLEᵥᵢᵢ | | Eₑ |
| 2046 | E_{1b}E₅EᵢᵢE₄ | AlkLEᵥᵢᵢ | | Eₑ |
| 2047 | E₁ₐE₅EᵢᵢE_{iib}E₄ | AlkLEᵥᵢᵢ | | Eₑ |
| 2048 | E_{1b}E₅EᵢᵢE_{iib}E₄ | AlkLEᵥᵢᵢ | | Eₑ |
| 2049 | E₁ₐEᵢᵢᵢ | AlkLEᵥᵢᵢ | | Eₑ |
| 2050 | E_{1b}Eᵢᵢᵢ | AlkLEᵥᵢᵢ | | Eₑ |
| 2051 | E₁ₐE₅Eᵢᵢᵢ | AlkLEᵥᵢᵢ | | Eₑ |
| 2052 | E_{1b}E₅Eᵢᵢᵢ | AlkLEᵥᵢᵢ | | Eₑ |
| 2053 | E₁ₐEᵢᵢᵢE₄ | AlkLEᵥᵢᵢ | | Eₑ |
| 2054 | E_{1b}EᵢᵢᵢE₄ | AlkLEᵥᵢᵢ | | Eₑ |
| 2055 | E₁ₐE₅EᵢᵢᵢE₄ | AlkLEᵥᵢᵢ | | Eₑ |
| 2056 | E_{1b}E₅EᵢᵢᵢE₄ | AlkLEᵥᵢᵢ | | Eₑ |
| 2057 | E₁ₐEᵢᵥ | AlkLEᵥᵢᵢ | | Eₑ |
| 2058 | E_{1b}Eᵢᵥ | AlkLEᵥᵢᵢ | | Eₑ |
| 2059 | E₁ₐE₅Eᵢᵥ | AlkLEᵥᵢᵢ | | Eₑ |
| 2060 | E_{1b}E₅Eᵢᵥ | AlkLEᵥᵢᵢ | | Eₑ |
| 2061 | E₁ₐEᵢᵥE₄ | AlkLEᵥᵢᵢ | | Eₑ |
| 2062 | E_{1b}EᵢᵥE₄ | AlkLEᵥᵢᵢ | | Eₑ |
| 2063 | E₁ₐE₅EᵢᵥE₄ | AlkLEᵥᵢᵢ | | Eₑ |
| 2064 | E_{1b}E₅EᵢᵥE₄ | AlkLEᵥᵢᵢ | | Eₑ |
| 2065 | E₁ₐ | AlkLEᵥᵢᵢ | E₄* | Eₑ |
| 2066 | E_{1b} | AlkLEᵥᵢᵢ | E₄* | Eₑ |
| 2067 | E₁ₐE₅ | AlkLEᵥᵢᵢ | E₄* | Eₑ |
| 2068 | E_{1b}E₅ | AlkLEᵥᵢᵢ | E₄* | Eₑ |
| 2069 | E₁ₐEᵢ | AlkLEᵥᵢᵢ | E₄* | Eₑ |
| 2070 | E_{1b}Eᵢ | AlkLEᵥᵢᵢ | E₄* | Eₑ |
| 2071 | E₁ₐEᵢᵢ | AlkLEᵥᵢᵢ | E₄* | Eₑ |
| 2072 | E_{1b}Eᵢᵢ | AlkLEᵥᵢᵢ | E₄* | Eₑ |
| 2073 | E₁ₐEᵢᵢE_{iib} | AlkLEᵥᵢᵢ | E₄* | Eₑ |
| 2074 | E_{1b}EᵢᵢE_{iib} | AlkLEᵥᵢᵢ | E₄* | Eₑ |
| 2075 | E₁ₐE₅Eᵢ | AlkLEᵥᵢᵢ | E₄* | Eₑ |
| 2076 | E_{1b}E₅Eᵢ | AlkLEᵥᵢᵢ | E₄* | Eₑ |
| 2077 | E₁ₐE₅Eᵢᵢ | AlkLEᵥᵢᵢ | E₄* | Eₑ |
| 2078 | E_{1b}E₅Eᵢᵢ | AlkLEᵥᵢᵢ | E₄* | Eₑ |
| 2079 | E₁ₐE₅EᵢᵢE_{iib} | AlkLEᵥᵢᵢ | E₄* | Eₑ |
| 2080 | E_{1b}E₅EᵢᵢE_{iib} | AlkLEᵥᵢᵢ | E₄* | Eₑ |
| 2081 | E₁ₐE₄ | AlkLEᵥᵢᵢ | E₄* | Eₑ |
| 2082 | E_{1b}E₄ | AlkLEᵥᵢᵢ | E₄* | Eₑ |
| 2083 | E₁ₐE₅E₄ | AlkLEᵥᵢᵢ | E₄* | Eₑ |
| 2084 | E_{1b}E₅E₄ | AlkLEᵥᵢᵢ | E₄* | Eₑ |
| 2085 | E₁ₐEᵢE₄ | AlkLEᵥᵢᵢ | E₄* | Eₑ |
| 2086 | E_{1b}EᵢE₄ | AlkLEᵥᵢᵢ | E₄* | Eₑ |
| 2087 | E₁ₐEᵢᵢE₄ | AlkLEᵥᵢᵢ | E₄* | Eₑ |
| 2088 | E_{1b}EᵢᵢE₄ | AlkLEᵥᵢᵢ | E₄* | Eₑ |
| 2089 | E₁ₐEᵢᵢE_{iib}E₄ | AlkLEᵥᵢᵢ | E₄* | Eₑ |
| 2090 | E_{1b}EᵢᵢE_{iib}E₄ | AlkLEᵥᵢᵢ | E₄* | Eₑ |
| 2091 | E₁ₐE₅EᵢE₄ | AlkLEᵥᵢᵢ | E₄* | Eₑ |
| 2092 | E_{1b}E₅EᵢE₄ | AlkLEᵥᵢᵢ | E₄* | Eₑ |
| 2093 | E₁ₐE₅EᵢᵢE₄ | AlkLEᵥᵢᵢ | E₄* | Eₑ |
| 2094 | E_{1b}E₅EᵢᵢE₄ | AlkLEᵥᵢᵢ | E₄* | Eₑ |
| 2095 | E₁ₐE₅EᵢᵢE_{iib}E₄ | AlkLEᵥᵢᵢ | E₄* | Eₑ |
| 2096 | E_{1b}E₅EᵢᵢE_{iib}E₄ | AlkLEᵥᵢᵢ | E₄* | Eₑ |
| 2097 | E₁ₐEᵢᵢᵢ | AlkLEᵥᵢᵢ | E₄* | Eₑ |
| 2098 | E_{1b}Eᵢᵢᵢ | AlkLEᵥᵢᵢ | E₄* | Eₑ |
| 2099 | E₁ₐE₅Eᵢᵢᵢ | AlkLEᵥᵢᵢ | E₄* | Eₑ |
| 2100 | E_{1b}E₅Eᵢᵢᵢ | AlkLEᵥᵢᵢ | E₄* | Eₑ |
| 2101 | E₁ₐEᵢᵢᵢE₄ | AlkLEᵥᵢᵢ | E₄* | Eₑ |
| 2102 | E_{1b}EᵢᵢᵢE₄ | AlkLEᵥᵢᵢ | E₄* | Eₑ |
| 2103 | E₁ₐE₅EᵢᵢᵢE₄ | AlkLEᵥᵢᵢ | E₄* | Eₑ |
| 2104 | E_{1b}E₅EᵢᵢᵢE₄ | AlkLEᵥᵢᵢ | E₄* | Eₑ |
| 2105 | E₁ₐEᵢᵥ | AlkLEᵥᵢᵢ | E₄* | Eₑ |
| 2106 | E_{1b}Eᵢᵥ | AlkLEᵥᵢᵢ | E₄* | Eₑ |
| 2107 | E₁ₐE₅Eᵢᵥ | AlkLEᵥᵢᵢ | E₄* | Eₑ |
| 2108 | E_{1b}E₅Eᵢᵥ | AlkLEᵥᵢᵢ | E₄* | Eₑ |
| 2109 | E₁ₐEᵢᵥE₄ | AlkLEᵥᵢᵢ | E₄* | Eₑ |
| 2110 | E_{1b}EᵢᵥE₄ | AlkLEᵥᵢᵢ | E₄* | Eₑ |
| 2111 | E₁ₐE₅EᵢᵥE₄ | AlkLEᵥᵢᵢ | E₄* | Eₑ |
| 2112 | E_{1b}E₅EᵢᵥE₄ | AlkLEᵥᵢᵢ | E₄* | Eₑ |
| 2113 | E₁ₐ | Eᵥᵢᵢ | | E_{f} |
| 2114 | E_{1b} | Eᵥᵢᵢ | | E_{f} |
| 2115 | E₁ₐE₅ | Eᵥᵢᵢ | | E_{f} |
| 2116 | E_{1b}E₅ | Eᵥᵢᵢ | | E_{f} |
| 2117 | E₁ₐEᵢ | Eᵥᵢᵢ | | E_{f} |
| 2118 | E_{1b}Eᵢ | Eᵥᵢᵢ | | E_{f} |
| 2119 | E₁ₐEᵢᵢ | Eᵥᵢᵢ | | E_{f} |
| 2120 | E_{1b}Eᵢᵢ | Eᵥᵢᵢ | | E_{f} |
| 2121 | E₁ₐEᵢᵢE_{iib} | Eᵥᵢᵢ | | E_{f} |
| 2122 | E_{1b}EᵢᵢE_{iib} | Eᵥᵢᵢ | | E_{f} |
| 2123 | E₁ₐE₅Eᵢ | Eᵥᵢᵢ | | E_{f} |
| 2124 | E_{1b}E₅Eᵢ | Eᵥᵢᵢ | | E_{f} |
| 2125 | E₁ₐE₅Eᵢᵢ | Eᵥᵢᵢ | | E_{f} |
| 2126 | E_{1b}E₅Eᵢᵢ | Eᵥᵢᵢ | | E_{f} |
| 2127 | E₁ₐE₅EᵢᵢE_{iib} | Eᵥᵢᵢ | | E_{f} |
| 2128 | E_{1b}E₅EᵢᵢE_{iib} | Eᵥᵢᵢ | | E_{f} |
| 2129 | E₁ₐE₄ | Eᵥᵢᵢ | | E_{f} |
| 2130 | E_{1b}E₄ | Eᵥᵢᵢ | | E_{f} |
| 2131 | E₁ₐE₅E₄ | Eᵥᵢᵢ | | E_{f} |
| 2132 | E_{1b}E₅E₄ | Eᵥᵢᵢ | | E_{f} |
| 2133 | E₁ₐEᵢE₄ | Eᵥᵢᵢ | | E_{f} |
| 2134 | E_{1b}EᵢE₄ | Eᵥᵢᵢ | | E_{f} |
| 2135 | E₁ₐEᵢᵢE₄ | Eᵥᵢᵢ | | E_{f} |
| 2136 | E_{1b}EᵢᵢE₄ | Eᵥᵢᵢ | | E_{f} |
| 2137 | E₁ₐEᵢᵢE_{iib}E₄ | Eᵥᵢᵢ | | E_{f} |
| 2138 | E_{1b}EᵢᵢE_{iib}E₄ | Eᵥᵢᵢ | | E_{f} |
| 2139 | E₁ₐE₅EᵢE₄ | Eᵥᵢᵢ | | E_{f} |
| 2140 | E_{1b}E₅EᵢE₄ | Eᵥᵢᵢ | | E_{f} |
| 2141 | E₁ₐE₅EᵢᵢE₄ | Eᵥᵢᵢ | | E_{f} |
| 2142 | E_{1b}E₅EᵢᵢE₄ | Eᵥᵢᵢ | | E_{f} |
| 2143 | E₁ₐE₅EᵢᵢE_{iib}E₄ | Eᵥᵢᵢ | | E_{f} |
| 2144 | E_{1b}E₅EᵢᵢE_{iib}E₄ | Eᵥᵢᵢ | | E_{f} |
| 2145 | E₁ₐEᵢᵢᵢ | Eᵥᵢᵢ | | E_{f} |
| 2146 | E_{1b}Eᵢᵢᵢ | Eᵥᵢᵢ | | E_{f} |
| 2147 | E₁ₐE₅Eᵢᵢᵢ | Eᵥᵢᵢ | | E_{f} |
| 2148 | E_{1b}E₅Eᵢᵢᵢ | Eᵥᵢᵢ | | E_{f} |
| 2149 | E₁ₐEᵢᵢᵢE₄ | Eᵥᵢᵢ | | E_{f} |
| 2150 | E_{1b}EᵢᵢᵢE₄ | Eᵥᵢᵢ | | E_{f} |
| 2151 | E₁ₐE₅EᵢᵢᵢE₄ | Eᵥᵢᵢ | | E_{f} |
| 2152 | E_{1b}E₅EᵢᵢᵢE₄ | Eᵥᵢᵢ | | E_{f} |
| 2153 | E₁ₐEᵢᵥ | Eᵥᵢᵢ | | E_{f} |
| 2154 | E_{1b}Eᵢᵥ | Eᵥᵢᵢ | | E_{f} |
| 2155 | E₁ₐE₅Eᵢᵥ | Eᵥᵢᵢ | | E_{f} |
| 2156 | E_{1b}E₅Eᵢᵥ | Eᵥᵢᵢ | | E_{f} |
| 2157 | E₁ₐEᵢᵥE₄ | Eᵥᵢᵢ | | E_{f} |
| 2158 | E_{1b}EᵢᵥE₄ | Eᵥᵢᵢ | | E_{f} |
| 2159 | E₁ₐE₅EᵢᵥE₄ | Eᵥᵢᵢ | | E_{f} |
| 2160 | E_{1b}E₅EᵢᵥE₄ | Eᵥᵢᵢ | | E_{f} |
| 2161 | E₁ₐ | Eᵥᵢᵢ | E₄* | E_{f} |
| 2162 | E_{1b} | Eᵥᵢᵢ | E₄* | E_{f} |
| 2163 | E₁ₐE₅ | Eᵥᵢᵢ | E₄* | E_{f} |
| 2164 | E_{1b}E₅ | Eᵥᵢᵢ | E₄* | E_{f} |
| 2165 | E₁ₐEᵢ | Eᵥᵢᵢ | E₄* | E_{f} |
| 2166 | E_{1b}Eᵢ | Eᵥᵢᵢ | E₄* | E_{f} |
| 2167 | E₁ₐEᵢᵢ | Eᵥᵢᵢ | E₄* | E_{f} |
| 2168 | E_{1b}Eᵢᵢ | Eᵥᵢᵢ | E₄* | E_{f} |
| 2169 | E₁ₐEᵢᵢE_{iib} | Eᵥᵢᵢ | E₄* | E_{f} |
| 2170 | E_{1b}EᵢᵢE_{iib} | Eᵥᵢᵢ | E₄* | E_{f} |
| 2171 | E₁ₐE₅Eᵢ | Eᵥᵢᵢ | E₄* | E_{f} |
| 2172 | E_{1b}E₅Eᵢ | Eᵥᵢᵢ | E₄* | E_{f} |
| 2173 | E₁ₐE₅Eᵢᵢ | Eᵥᵢᵢ | E₄* | E_{f} |
| 2174 | E_{1b}E₅Eᵢᵢ | Eᵥᵢᵢ | E₄* | E_{f} |
| 2175 | E₁ₐE₅EᵢᵢE_{iib} | Eᵥᵢᵢ | E₄* | E_{f} |
| 2176 | E_{1b}E₅EᵢᵢE_{iib} | Eᵥᵢᵢ | E₄* | E_{f} |
| 2177 | E₁ₐE₄ | Eᵥᵢᵢ | E₄* | E_{f} |
| 2178 | E_{1b}E₄ | Eᵥᵢᵢ | E₄* | E_{f} |
| 2179 | E₁ₐE₅E₄ | Eᵥᵢᵢ | E₄* | E_{f} |
| 2180 | E_{1b}E₅E₄ | Eᵥᵢᵢ | E₄* | E_{f} |
| 2181 | E₁ₐEᵢE₄ | Eᵥᵢᵢ | E₄* | E_{f} |
| 2182 | E_{1b}EᵢE₄ | Eᵥᵢᵢ | E₄* | E_{f} |
| 2183 | E₁ₐEᵢᵢE₄ | Eᵥᵢᵢ | E₄* | E_{f} |
| 2184 | E_{1b}EᵢᵢE₄ | Eᵥᵢᵢ | E₄* | E_{f} |
| 2185 | E₁ₐEᵢᵢE_{iib}E₄ | Eᵥᵢᵢ | E₄* | E_{f} |
| 2186 | E_{1b}EᵢᵢE_{iib}E₄ | Eᵥᵢᵢ | E₄* | E_{f} |
| 2187 | E₁ₐE₅EᵢE₄ | Eᵥᵢᵢ | E₄* | E_{f} |
| 2188 | E_{1b}E₅EᵢE₄ | Eᵥᵢᵢ | E₄* | E_{f} |
| 2189 | E₁ₐE₅EᵢᵢE₄ | Eᵥᵢᵢ | E₄* | E_{f} |
| 2190 | E_{1b}E₅EᵢᵢE₄ | Eᵥᵢᵢ | E₄* | E_{f} |
| 2191 | E₁ₐE₅EᵢᵢE_{iib}E₄ | Eᵥᵢᵢ | E₄* | E_{f} |
| 2192 | E_{1b}E₅EᵢᵢE_{iib}E₄ | Eᵥᵢᵢ | E₄* | E_{f} |
| 2193 | E₁ₐEᵢᵢᵢ | Eᵥᵢᵢ | E₄* | E_{f} |
| 2194 | E_{1b}Eᵢᵢᵢ | Eᵥᵢᵢ | E₄* | E_{f} |
| 2195 | E₁ₐE₅Eᵢᵢᵢ | Eᵥᵢᵢ | E₄* | E_{f} |
| 2196 | E_{1b}E₅Eᵢᵢᵢ | Eᵥᵢᵢ | E₄* | E_{f} |
| 2197 | E₁ₐEᵢᵢᵢE₄ | Eᵥᵢᵢ | E₄* | E_{f} |
| 2198 | E_{1b}EᵢᵢᵢE₄ | Eᵥᵢᵢ | E₄* | E_{f} |
| 2199 | E₁ₐE₅EᵢᵢᵢE₄ | Eᵥᵢᵢ | E₄* | E_{f} |
| 2200 | E_{1b}E₅EᵢᵢᵢE₄ | Eᵥᵢᵢ | E₄* | E_{f} |
| 2201 | E₁ₐEᵢᵥ | Eᵥᵢᵢ | E₄* | E_{f} |
| 2202 | E_{1b}Eᵢᵥ | Eᵥᵢᵢ | E₄* | E_{f} |
| 2203 | E₁ₐE₅Eᵢᵥ | Eᵥᵢᵢ | E₄* | E_{f} |
| 2204 | E_{1b}E₅Eᵢᵥ | Eᵥᵢᵢ | E₄* | E_{f} |
| 2205 | E₁ₐEᵢᵥE₄ | Eᵥᵢᵢ | E₄* | E_{f} |
| 2206 | E_{1b}EᵢᵥE₄ | Eᵥᵢᵢ | E₄* | E_{f} |
| 2207 | E₁ₐE₅EᵢᵥE₄ | Eᵥᵢᵢ | E₄* | E_{f} |
| 2208 | E_{1b}E₅EᵢᵥE₄ | Eᵥᵢᵢ | E₄* | E_{f} |
| 2209 | E₁ₐ | AlkLEᵥᵢᵢ | | E_{f} |
| 2210 | E_{1b} | AlkLEᵥᵢᵢ | | E_{f} |
| 2211 | E₁ₐE₅ | AlkLEᵥᵢᵢ | | E_{f} |
| 2212 | E_{1b}E₅ | AlkLEᵥᵢᵢ | | E_{f} |
| 2213 | E₁ₐEᵢ | AlkLEᵥᵢᵢ | | E_{f} |
| 2214 | E_{1b}Eᵢ | AlkLEᵥᵢᵢ | | E_{f} |
| 2215 | E₁ₐEᵢᵢ | AlkLEᵥᵢᵢ | | E_{f} |
| 2216 | E_{1b}Eᵢᵢ | AlkLEᵥᵢᵢ | | E_{f} |
| 2217 | E₁ₐEᵢᵢE_{iib} | AlkLEᵥᵢᵢ | | E_{f} |
| 2218 | E_{1b}EᵢᵢE_{iib} | AlkLEᵥᵢᵢ | | E_{f} |
| 2219 | E₁ₐE₅Eᵢ | AlkLEᵥᵢᵢ | | E_{f} |
| 2220 | E_{1b}E₅Eᵢ | AlkLEᵥᵢᵢ | | E_{f} |
| 2221 | E₁ₐE₅Eᵢᵢ | AlkLEᵥᵢᵢ | | E_{f} |
| 2222 | E_{1b}E₅Eᵢᵢ | AlkLEᵥᵢᵢ | | E_{f} |
| 2223 | E₁ₐE₅EᵢᵢE_{iib} | AlkLEᵥᵢᵢ | | E_{f} |
| 2224 | E_{1b}E₅EᵢᵢE_{iib} | AlkLEᵥᵢᵢ | | E_{f} |
| 2225 | E₁ₐE₄ | AlkLEᵥᵢᵢ | | E_{f} |
| 2226 | E_{1b}E₄ | AlkLEᵥᵢᵢ | | E_{f} |
| 2227 | E₁ₐE₅E₄ | AlkLEᵥᵢᵢ | | E_{f} |
| 2228 | E_{1b}E₅E₄ | AlkLEᵥᵢᵢ | | E_{f} |
| 2229 | E₁ₐEᵢE₄ | AlkLEᵥᵢᵢ | | E_{f} |
| 2230 | E_{1b}EᵢE₄ | AlkLEᵥᵢᵢ | | E_{f} |
| 2231 | E₁ₐEᵢᵢE₄ | AlkLEᵥᵢᵢ | | E_{f} |
| 2232 | E_{1b}EᵢᵢE₄ | AlkLEᵥᵢᵢ | | E_{f} |
| 2233 | E₁ₐEᵢᵢE_{iib}E₄ | AlkLEᵥᵢᵢ | | E_{f} |
| 2234 | E_{1b}EᵢᵢE_{iib}E₄ | AlkLEᵥᵢᵢ | | E_{f} |
| 2235 | E₁ₐE₅EᵢE₄ | AlkLEᵥᵢᵢ | | E_{f} |
| 2236 | E_{1b}E₅EᵢE₄ | AlkLEᵥᵢᵢ | | E_{f} |
| 2237 | E₁ₐE₅EᵢᵢE₄ | AlkLEᵥᵢᵢ | | E_{f} |
| 2238 | E_{1b}E₅EᵢᵢE₄ | AlkLEᵥᵢᵢ | | E_{f} |
| 2239 | E₁ₐE₅EᵢᵢE_{iib}E₄ | AlkLEᵥᵢᵢ | | E_{f} |
| 2240 | E_{1b}E₅EᵢᵢE_{iib}E₄ | AlkLEᵥᵢᵢ | | E_{f} |
| 2241 | E₁ₐEᵢᵢᵢ | AlkLEᵥᵢᵢ | | E_{f} |
| 2242 | E_{1b}Eᵢᵢᵢ | AlkLEᵥᵢᵢ | | E_{f} |
| 2243 | E₁ₐE₅Eᵢᵢᵢ | AlkLEᵥᵢᵢ | | E_{f} |
| 2244 | E_{1b}E₅Eᵢᵢᵢ | AlkLEᵥᵢᵢ | | E_{f} |
| 2245 | E₁ₐEᵢᵢᵢE₄ | AlkLEᵥᵢᵢ | | E_{f} |
| 2246 | E_{1b}EᵢᵢᵢE₄ | AlkLEᵥᵢᵢ | | E_{f} |
| 2247 | E₁ₐE₅EᵢᵢᵢE₄ | AlkLEᵥᵢᵢ | | E_{f} |
| 2248 | E_{1b}E₅EᵢᵢᵢE₄ | AlkLEᵥᵢᵢ | | E_{f} |
| 2249 | E₁ₐEᵢᵥ | AlkLEᵥᵢᵢ | | E_{f} |
| 2250 | E_{1b}Eᵢᵥ | AlkLEᵥᵢᵢ | | E_{f} |
| 2251 | E₁ₐE₅Eᵢᵥ | AlkLEᵥᵢᵢ | | E_{f} |
| 2252 | E_{1b}E₅Eᵢᵥ | AlkLEᵥᵢᵢ | | E_{f} |
| 2253 | E₁ₐEᵢᵥE₄ | AlkLEᵥᵢᵢ | | E_{f} |
| 2254 | E_{1b}EᵢᵥE₄ | AlkLEᵥᵢᵢ | | E_{f} |
| 2255 | E₁ₐE₅EᵢᵥE₄ | AlkLEᵥᵢᵢ | | E_{f} |
| 2256 | E_{1b}E₅EᵢᵥE₄ | AlkLEᵥᵢᵢ | | E_{f} |
| 2257 | E₁ₐ | AlkLEᵥᵢᵢ | E₄* | E_{f} |
| 2258 | E_{1b} | AlkLEᵥᵢᵢ | E₄* | E_{f} |
| 2259 | E₁ₐE₅ | AlkLEᵥᵢᵢ | E₄* | E_{f} |
| 2260 | E_{1b}E₅ | AlkLEᵥᵢᵢ | E₄* | E_{f} |
| 2261 | E₁ₐEᵢ | AlkLEᵥᵢᵢ | E₄* | E_{f} |
| 2262 | E_{1b}Eᵢ | AlkLEᵥᵢᵢ | E₄* | E_{f} |
| 2263 | E₁ₐEᵢᵢ | AlkLEᵥᵢᵢ | E₄* | E_{f} |
| 2264 | E_{1b}Eᵢᵢ | AlkLEᵥᵢᵢ | E₄* | E_{f} |
| 2265 | E₁ₐEᵢᵢE_{iib} | AlkLEᵥᵢᵢ | E₄* | E_{f} |
| 2266 | E_{1b}EᵢᵢE_{iib} | AlkLEᵥᵢᵢ | E₄* | E_{f} |
| 2267 | E₁ₐE₅Eᵢ | AlkLEᵥᵢᵢ | E₄* | E_{f} |
| 2268 | E_{1b}E₅Eᵢ | AlkLEᵥᵢᵢ | E₄* | E_{f} |
| 2269 | E₁ₐE₅Eᵢᵢ | AlkLEᵥᵢᵢ | E₄* | E_{f} |
| 2270 | E_{1b}E₅Eᵢᵢ | AlkLEᵥᵢᵢ | E₄* | E_{f} |
| 2271 | E₁ₐE₅EᵢᵢE_{iib} | AlkLEᵥᵢᵢ | E₄* | E_{f} |
| 2272 | E_{1b}E₅EᵢᵢE_{iib} | AlkLEᵥᵢᵢ | E₄* | E_{f} |
| 2273 | E₁ₐE₄ | AlkLEᵥᵢᵢ | E₄* | E_{f} |
| 2274 | E_{1b}E₄ | AlkLEᵥᵢᵢ | E₄* | E_{f} |
| 2275 | E₁ₐE₅E₄ | AlkLEᵥᵢᵢ | E₄* | E_{f} |
| 2276 | E_{1b}E₅E₄ | AlkLEᵥᵢᵢ | E₄* | E_{f} |
| 2277 | E₁ₐEᵢE₄ | AlkLEᵥᵢᵢ | E₄* | E_{f} |
| 2278 | E_{1b}EᵢE₄ | AlkLEᵥᵢᵢ | E₄* | E_{f} |
| 2279 | E₁ₐEᵢᵢE₄ | AlkLEᵥᵢᵢ | E₄* | E_{f} |
| 2280 | E_{1b}EᵢᵢE₄ | AlkLEᵥᵢᵢ | E₄* | E_{f} |
| 2281 | E₁ₐEᵢᵢE_{iib}E₄ | AlkLEᵥᵢᵢ | E₄* | E_{f} |
| 2282 | E_{1b}EᵢᵢE_{iib}E₄ | AlkLEᵥᵢᵢ | E₄* | E_{f} |
| 2283 | E₁ₐE₅EᵢE₄ | AlkLEᵥᵢᵢ | E₄* | E_{f} |
| 2284 | E_{1b}E₅EᵢE₄ | AlkLEᵥᵢᵢ | E₄* | E_{f} |
| 2285 | E₁ₐE₅EᵢᵢE₄ | AlkLEᵥᵢᵢ | E₄* | E_{f} |
| 2286 | E_{1b}E₅EᵢᵢE₄ | AlkLEᵥᵢᵢ | E₄* | E_{f} |
| 2287 | E₁ₐE₅EᵢᵢE_{iib}E₄ | AlkLEᵥᵢᵢ | E₄* | E_{f} |
| 2288 | E_{1b}E₅EᵢᵢE_{iib}E₄ | AlkLEᵥᵢᵢ | E₄* | E_{f} |
| 2289 | E₁ₐEᵢᵢᵢ | AlkLEᵥᵢᵢ | E₄* | E_{f} |
| 2290 | E_{1b}Eᵢᵢᵢ | AlkLEᵥᵢᵢ | E₄* | E_{f} |
| 2291 | E₁ₐE₅Eᵢᵢᵢ | AlkLEᵥᵢᵢ | E₄* | E_{f} |
| 2292 | E_{1b}E₅Eᵢᵢᵢ | AlkLEᵥᵢᵢ | E₄* | E_{f} |
| 2293 | E₁ₐEᵢᵢᵢE₄ | AlkLEᵥᵢᵢ | E₄* | E_{f} |
| 2294 | E_{1b}EᵢᵢᵢE₄ | AlkLEᵥᵢᵢ | E₄* | E_{f} |
| 2295 | E₁ₐE₅EᵢᵢᵢE₄ | AlkLEᵥᵢᵢ | E₄* | E_{f} |
| 2296 | E_{1b}E₅EᵢᵢᵢE₄ | AlkLEᵥᵢᵢ | E₄* | E_{f} |
| 2297 | E₁ₐEᵢᵥ | AlkLEᵥᵢᵢ | E₄* | E_{f} |
| 2298 | E_{1b}Eᵢᵥ | AlkLEᵥᵢᵢ | E₄* | E_{f} |
| 2299 | E₁ₐE₅Eᵢᵥ | AlkLEᵥᵢᵢ | E₄* | E_{f} |
| 2300 | E_{1b}E₅Eᵢᵥ | AlkLEᵥᵢᵢ | E₄* | E_{f} |
| 2301 | E₁ₐEᵢᵥE₄ | AlkLEᵥᵢᵢ | E₄* | E_{f} |
| 2302 | E_{1b}EᵢᵥE₄ | AlkLEᵥᵢᵢ | E₄* | E_{f} |
| 2303 | E₁ₐE₅EᵢᵥE₄ | AlkLEᵥᵢᵢ | E₄* | E_{f} |
| 2304 | E_{1b}E₅EᵢᵥE₄ | AlkLEᵥᵢᵢ | E₄* | E_{f} |

### Use of the Microorganisms According to the Invention

A further subject of the present invention relates to the use of the aforesaid microorganisms for the production of ω-functionalized carboxylic acids and ω-functionalized carboxylate esters, in particular of those carboxylic acids and carboxylate esters, which were emphasized above as preferable in connection with the microorganisms according to the invention, wherein as the ω-functionalization, ω-amination in particular is to be emphasized. The use of the aforesaid microorganisms for the production of ω-aminocarboxylic acids and ω-aminocarboxylate esters, in particular ω-aminolauric acid and methyl and ethyl ω-aminolaurates and ω-aminocaproic acid and methyl and ethyl ω-aminocaproates is particularly preferable.

Microorganisms emphasized as preferable in connection with the microorganisms according to the invention are also preferable in connection with the use according to the invention. Which organisms according to the invention are preferable for specific ω-functionalized carboxylic acids or ω-functionalized carboxylate esters has already been emphasized in connection with the microorganisms according to the invention.

Process for the Production of ω-Functionalized Carboxylic Acids and of ω-Functionalized carboxylate esters

A further subject of the present invention relates to a process for the production of ω-functionalized carboxylic acids and ω-functionalized carboxylate esters from a simple carbon source comprising the process steps

I) contacting a microorganism according to the invention with a medium containing the simple carbon source,
II) culturing the microorganism under conditions which enable the microorganism to form the ω-functionalized carboxylic acids or ω-functionalized carboxylate esters from the simple carbon source and
III) optionally isolation of the ω-functionalized carboxylic acids or ω-functionalized carboxylate esters formed.

In the process according to the invention, the microorganisms according to the invention can be contacted with the culture medium and cultured continuously or discontinuously in the batch process (batch culturing) or the fed-batch process or the repeated fed-batch process for the purpose of producing the ω-functionalized carboxylic acids or ω-functionalized carboxylate esters. Also possible is a semicontinuous process as described in GB-A-1009370. A summary of known culturing methods is described in the textbook by Chmiel (“Bioprozesstechnik 1. Einführung in die Bioverfahrenstechnik”, Gustav Fischer Verlag, Stuttgart, 1991) or in the textbook by Storhas (“Bioreaktoren and periphere Einrichtungen”, Vieweg Verlag, Braunschweig/Wiesbaden, 1994).

The culture medium to be used must appropriately meet the requirements of the respective strains. Descriptions of culture media for various microorganisms are contained in the American Society for Bacteriology manual “Manual of Methods for General Bacteriology” (Washington D.C., USA, 1981).

In the process according to the invention, microorganisms preferred according to the invention are preferably used.

As the simple carbon source in the process according to the invention, those mentioned above as preferable are used.

As the nitrogen source, organic nitrogen-containing compounds such as peptone, yeast extract, meat extract, malt extract, corn steep liquor, soya bean meal and urea or inorganic compounds such as ammonium sulphate, ammonium chloride, ammonium phosphate, ammonium carbonate and ammonium nitrate, ammonia, ammonium hydroxide or aqueous ammonia can be used. The nitrogen sources can be used singly or as a mixture.

As the phosphorus source, phosphoric acid, potassium dihydrogen phosphate or dipotassium hydrogen phosphate or the corresponding sodium-containing salts can be used. The culture medium must further contain salts of metals, such as for example magnesium sulphate or iron sulphate, which are necessary for growth. Finally, essential nutrients such as amino acids and vitamins can be used in addition to the abovementioned substances. Apart from this, suitable precursors can be added to the culture medium. The said additives can be added to the culture in the form of a single preparation or fed in during the culturing in a suitable manner.

For pH control of the culture, basic compounds such as sodium hydroxide, potassium hydroxide, ammonia or aqueous ammonia or acidic compounds such as phosphoric acid or sulphuric acid can be used in a suitable manner. To control foam development, antifoaming agents such as for example fatty acid polyglycol esters can be used. For maintenance of the stability of plasmids, suitable selectively acting substances such as for example antibiotics can be added to the media. In order to maintain aerobic conditions, oxygen or oxygen-containing gas mixtures such as for example air are introduced into the culture.

According to one embodiment of the process according to the invention, this is performed in a two-phase system, comprising

A) an aqueous phase, and
B) an organic phase,
wherein the formation of the ω-functionalized carboxylic acids or ω-functionalized carboxylate esters by the microorganism in process step II) takes place in the aqueous phase and the ω-functionalized carboxylic acids or ω-functionalized carboxylate esters formed accumulate in the organic phase. In this manner, it is possible to extract the ω-functionalized carboxylic acids or ω-functionalized carboxylate esters formed in situ.

Preferred ω-functionalized carboxylic acids or ω-functionalized carboxylate esters which are produced with the process according to the invention are those mentioned above as preferred in connection with the microorganisms according to the invention and the use according to the invention.

Especially preferred ω-functionalized carboxylic acids or ω-functionalized carboxylate esters are the

ω-aminocarboxylic acids and ω-aminocarboxylate esters, in particular ω-aminolauric acid and methyl and ethyl ω-aminolaurates and ω-aminocaproic acid and methyl and ethyl ω-aminocaproates, and
ω-hydroxycarboxylic acids and ω-hydroxycarboxylate esters, in particular ω-hydroxylauric acid and methyl and ethyl ω-hydroxylaurates and ω-hydroxycaproic acid and methyl and ethyl ω-hydroxycaproates, and
ω-carboxycarboxylic acids and ω-carboxycarboxylate esters, in particular ω-carboxylauric acid and methyl and ethyl ω-carboxylaurates, and ω-carboxycaproic acid and methyl and ethyl ω-carboxycaproates.

Which organisms according to the invention are preferably used in preferred processes according to the invention for specific ω-functionalized carboxylic acids or ω-functionalized carboxylate esters has already been emphasized in connection with the microorganisms according to the invention.

A further subject of the present invention is a process for the production of polyamides based on ω-aminocarboxylic acids, comprising the process steps:

(a1) Production of ω-aminocarboxylic acids or ω-aminocarboxylate esters by one of the processes described above for the production of ω-aminocarboxylic acids, in particular by the process described above for the production of ω-aminolauric acid, methyl ω-aminolaurate, ω-aminocaproic acid or methyl ω-aminocaproate and optionally conversion of the ω-aminocarboxylate esters into ω-aminocarboxylic acids;
(a2) Polymerization of the ω-aminocarboxylic acid with obtention of a polyamide.

In process step (a2) of the process according to the invention for the production of polyamides based on ω-aminocarboxylic acids, the ω-aminocarboxylate esters can be converted into the ω-aminocarboxylic acids by any processes such as for example acid- or base-catalysed hydrolysis.

In process step (a2) of the process according to the invention for the production of polyamides based on ω-aminocarboxylic acids, the ω-aminocarboxylic acids obtained in process step (a1), in particular the ω-aminolauric acid obtained in process step (a1), is converted to a polyamide in a polymerization wherein optionally mixtures of various ω-aminocarboxylic acids can also be used, whereof at least a part of the ω-aminocarboxylic acids, preferably at least 50 wt. % based on all ω-aminocarboxylic acids used in the process, but optionally also all ω-aminocarboxylic acids were produced by the process according to the invention for the production of ω-aminocarboxylic acids.

The production of the polyamides from the ω-aminocarboxylic acids can be effected in processes known per se, as for example described in L. Notarbartolo, Ind. Plast. Mod. 10 (1958) 2, p. 44, JP 01-074224, JP 01-051433, JP63286428, JP58080324 or JP60179425.

In the examples presented below, the present invention is described by way of example, without it being intended that the invention, the scope of whose application appears from the whole description and claims, be limited to the embodiments mentioned in the examples.

### EXAMPLES

### Example 1

### Production of Expression Vectors for the Genes fatB2 from Cuphea palustris, Ald from Bacillus subtilis and Cv_{—}2025 from Chromobacterium violaceum

For the production of an E. coli expression vector for the genes fatB2 (SEQ ID No. 10) from Cuphea palustris (enzyme Eᵢ), aid (SEQ ID No. 11) from Bacillus subtilis (enzyme E₃) and Cv_{—}2025 (SEQ ID No. 12) from Chromobacterium violaceum (enzyme E₂), these genes were successively cloned into the vector pJ294 (DNA2.0 Inc., Menlo Park, Calif., USA). The gene Cv_{—}2025 was synthesized together with a lacUV5 promoter and the gene ald from Bacillus sphaericus and simultaneously a cleavage site upstream of the promoter and a cleavage site downstream of the terminator were introduced. The synthesized DNA fragment P_{lacUV5}-. ald_Bsp_TA_C.v.(Ct) (SEQ ID No. 13) was digested with the restriction endonucleases PstI and XbaI and ligated into the correspondingly cleaved vector pJ294. The finished E. coli expression vector was designated as pJ294_alaD_Bsp_TA_C.v.(Ct).(SEQ ID No. 14). In this vector, the Bacillus sphaericus ald gene was replaced by the gene ald from Bacillus subtilis. The gene ald was amplified by PCR from chromosomal DNA of the strain Bacillus subtilis str. 168. The following oligonucleotides were used in this:

| | |
|---|---|
| alaDH_pCR22_fw: | 5′-ATGATCATAGGGGTTCCTAAAGAG-3′ |
| | (SEQ ID No. 15) |
| alaDH_pCR22_rev: | 5′-TTAAGCACCCGCCACAGATG-3′ |
| | (SEQ ID No. 16) |

The following parameters were used for the PCR: 1×: initial denaturation, 98° C., 0:30 mins; 35×: denaturation, 98° C., 0:10 min, annealing, 65° C., 0:30 mins; elongation, 72° C., 0:20 mins; 1×: terminal elongation, 72° C., 10 min. For the amplification, the Phusion™ High-Fidelity Master Mix from New England Biolabs (Frankfurt) was used according to the manufacturer's recommendations. 50 μl of each PCR reaction were then separated on a 1% TAE agarose gel.

The PCR, agarose gel electrophoresis, ethidium bromide staining of the DNA and determination of the PCR fragment sizes were performed in the manner known to those skilled in the art. The PCR fragment exhibited the expected size of 1137 base-pairs and was purified from the PCR preparation with the Quick PCR Purification Kit from Qiagen (Hilden) according to the manufacturer's instructions. For the ligation of the PCR product with the vector, 5′-phosphates were attached to the PCR product by means of polynucleotide kinase (New England Biolabs, Frankfurt). For this, the manufacturer's recommendation was followed.

The vector was digested with the restriction endonucleases HindIII and NdeI, whereby the contained gene Bacillus sphaericus ald was removed. The restriction digestion mixture was separated on a 1% TAE agarose gel. Two bands, of sizes 5696 bp and 1124 bp could be identified. For the isolation of the vector-DNA from the agarose gel, the DNA band of 5696 bp was isolated from the gel with a scalpel and purified with the Quick Gel Extraction Kit from Qiagen (Hilden) according to the manufacturer's instructions. To create blunt ends, the 5′ overhangs of the purified vector DNA were filled in by means of the Klenow fragment of DNA polymerase I (New England Biolabs, Frankfurt). For this, the manufacturer's instructions were followed. The DNA fragment Bacillus subtilis ald with 5′ phosphate residues was ligated into the vector with blunt ends. The finished E. coli expression vector was designated as pJ294_alaDH_B.s._TA_C.v.(Ct) (SEQ ID No. 17).

For the production of the complete expression vector, the gene fatB2 from Cuphea palustris was codon-optimized for expression in Escherichia coli. The gene was synthesized together with a tac promoter (DNA 2.0; Menlo Park, Calif., USA) and simultaneously a cleavage site upstream of the promoter and a cleavage site downstream of the terminator were introduced.

The synthesized DNA fragment P_{tac}-CpFatB2 (SEQ ID No. 18) was digested with the restriction endonucleases BamHI and NotI and ligated into the correspondingly cleaved vector pJ294_alaDH_B.s._TA_C.v.(Ct) and the vector pJ294. The finished vectors were designated as pJ294[alaDH_B.s._TA_C.v.(Ct)_Ptac-CpFATB2] (SEQ ID No. 19) and pJ294[Ptac-CpFATB2_optEc] (SEQ ID No. 20).

### Example 2

### LC-ESI/MS²-Based Quantification of Products

The quantification of methyl laurate LSME, methyl ω-hydroxylaurate HLSME, methyl ω-oxolaurate OLSME, methyl ω-aminolaurate ALSME and methyl ω-carboxylaurate DDSME, ω-aminolauric acid ALS, ω-carboxylauric acid DDS, lauric acid LS, ω-hydroxylauric acid HLS and ω-oxolauric acid OLS in fermentation samples was effected by means of LC-ESI/MS² on the basis of an external calibration for all analytes and with use of the internal standard aminoundecanoic acid (AUD).

For this, the following instruments were used:
HPLC unit 1260 (Agilent; Böblingen) with autosampler (G1367E), binary pump (G1312B) and column oven (G1316A)
Mass spectrometer TripelQuad 6410 (Agilent; Böblingen) with ESI source
HPLC column: Kinetex C18, 100×2.1 mm, particle size: 2.6 μm, pore size 100 Å (Phenomenex; Aschaffenburg)
Precolumn: KrudKatcher Ultra HPLC in-line filter; 0.5 μm filter depth and 0.004 mm internal diameter (Phenomenex; Aschaffenburg)

The samples were prepared by pipetting 1900 μL of solvent (acetone/0.1 N HCl mixture=1:1) and 100 μL of sample into a 2 mL reaction vessel. The mixture was vortexed for ca. 10 seconds and then centrifuged at ca. 13,000 rpm for 5 mins. The clear supernatant was withdrawn with a pipette and analysed after appropriate dilution with diluent (80% (v/v) ACN, 20% bidest. H₂O (v/v), +0.1% formic acid). 100 μL of ISTD were pipetted into each 900 μL sample (10 μL with a sample volume of 90 μL).

The HPLC separation was effected with the aforementioned column and precolumn. The injection volume was 0.7 μL, the column temperature 50° C. and the flow rate 0.6 ml/min. The mobile phase consisted of eluent A (0.1% (v/v) aqueous formic acid) and eluent B (acetonitrile with 0.1% (v/v) formic acid). The following gradient profile was used

| Time [mins] | Eluent A [%] | Eluent B [%] |
|---|---|---|
| 0 | 77 | 23 |
| 0.3 | 77 | 23 |
| 0.4 | 40 | 60 |
| 2.5 | 40 | 60 |
| 2.6 | 2 | 98 |
| 5.5 | 2 | 98 |
| 5.6 | 77 | 23 |
| 9 | 77 | 23 |

The ESI-MS² analysis was effected in positive mode with the following ESI source parameters:
Gas temperature 280° C.
Gas flow rate 11 l/min
Nebulizer pressure 50 psi
Capillary voltage 4000 V

The detection and quantification of the individual compounds was effected with the following parameters, wherein in each case one product ion was used as Qualifier and one as Quantifier

| | Precursor | Product | Dwell | Collision |
|---|---|---|---|---|
| Analyte | Ion | Ion | time | energy |
| DDSME | 245.2 | 167.1 | 25 | 6 |
| DDSME | 245.2 | 149.1 | 50 | 8 |
| HLSME | 231.3 | 181.2 | 15 | 2 |
| HLSME | 231.3 | 163.2 | 25 | 5 |
| DDS | 231.2 | 213.2 | 50 | 0 |
| DDS | 231.2 | 149.1 | 25 | 9 |
| ALSME | 230.3 | 198.1 | 25 | 10 |
| ALSME | 230.3 | 163.2 | 15 | 10 |
| OLSME | 229.2 | 197.2 | 50 | 0 |
| OLSME | 229.2 | 161.1 | 25 | 5 |
| HLS | 217.2 | 181.2 | 35 | 0 |
| HLS | 217.2 | 163.1 | 20 | 4 |
| OLS | 215.2 | 161.2 | 25 | 0 |
| OLS | 215.2 | 95.2 | 60 | 13 |

### Example 3

### Production of Aminolauric Acid by an E. coli Strain with Deletion of the Gene fadE and Expression Vectors for the Genes fatB2 from Cuphea Palustris, Ald from Bacillus subtilis, and Cv_{—}2025 from Chromobacterium violaceum in Combination with an Expression Vector for the Genes alkB, alkG, alkT and alkL from the alk Operon of Pseudomonas putida

The host strain used E. coli JW5020-1 (CGSC, The coli genetic stock center, Yale University, New Haven, USA) is an E. coli BW25113 derivative which carries a deletion of the gene fadE (coding for enzyme E_{b}). The gene fadE was replaced by a kanamycin cassette. Before the provision of the strain with the expression vectors by means of a helper plasmid which codes for the Flp recombinase, this was removed in a manner known to those skilled in the art (see Datsenko K. A. and Wanner B. L. (2000) PNAS 97(12):6640-6645), resulting in the strain E. coli JW5020-1 Kan^{S}. To create an E. coli strain with expression vectors for the genes fatB2 from Cuphea palustris (enzyme Eᵢ), a/d from Bacillus subtilis (enzyme E₃), and Cv_{—}2025 from Chromobacterium violaceum (enzyme E₂) in combination with the expression vector pBT10_alkL (sequence and production: compare Example 1 of PCT/EP2011/053834 and the Seq ID No. 8 listed there) for the genes alkB (enzyme E₁ₐ), alkG, alkT (auxiliary enzymes to enzyme E_{1b}) and alkL (coding for alkL gene product) from the alk operon of Pseudomonas putida GPo1, electrocompetent cells of E. coli JW5020-1 Kan^{S} were produced. This took place in a manner known to those skilled in the art. This was transformed with the plasmids pJ294[alaDH_B.s._TA_C.v.(Ct)_Ptac-CpFATB2] and pBT10_alkL and plated out onto LB agar plates containing ampicillin (100 μg/ml) and kanamycin (50 μg/ml). Transformants were checked for the presence of the correct plasmids by plasmid preparation and analytical restriction analysis. In this manner, the following strain was constructed: E. coli JW5020-1 Kan^{S} pBT10_alkL/pJ294[alaDH_B.s._TA_C.v.(Ct)_Ptac-CpFATB2].

The strain was subjected to a fed-batch fermentation in order to analyse its capacity for the production of aminolauric acid from glucose. The strain to be tested was firstly grown from a glycerine culture as a preculture in M9 medium containing 100 μg/ml ampicillin and 50 μg/ml kanamycin at 37° C. overnight. The medium, consisting of 38 mM disodium hydrogen phosphate dihydrate, 22 mM potassium dihydrogen phosphate, 8.6 mM sodium chloride, 37 mM ammonium chloride, 1.5% (w/v) glucose, 2 mM magnesium sulphate heptahydrate (all substances from Merck, Darmstadt) and 0.5% (v/v) trace element solution, was adjusted to a pH of 7.4 with 25% ammonium hydroxide solution. The trace element solution added, consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 37% hydrochloric acid (all substances from Merck, Darmstadt) was sterile-filtered before addition to the M9 medium. The fermenter was inoculated with the preculture such that an optical density of 0.06 was reached. The culturing was effected at a pH of 6.8, regulated with 25% aqueous ammonia and 0.5 M sulphuric acid, an oxygen partial pressure of 20%, regulated via a stirrer speed of 800 rpm/min and air feed of 0.4 vvm/min at the start of the fermentation, and a temperature of 37° C. The glucose feed took place after consumption of the glucose present in the medium, at a feed rate of 5 g/l/hr based on the initial volume. At the start of the glucose feed after 9 hours fermentation, the temperature was adjusted to 30° C. The gene expression was induced 2 hours after the start of the glucose feed by addition of 1 mM isopropyl-β-D-thiogalactopyranoside and 0.025% dicyclopropyl ketone. The strain was cultured for a further 49 hours under constant conditions. During the culturing, 1 ml samples were withdrawn and the concentration of fatty acids and ω-functionalized fatty acids quantified by the method described in Example 2. The results are shown in the following table.

| C_{lauric acid} | C_{carboxylauric acid} | C_{hydroxylauric acid} | C_{aminolauric acid} |
|---|---|---|---|
| [mg/l] | [mg/l] | [mg/l] | [mg/l] |
| 56.5 | 16.0 | 54.4 | 22.8 |

Production of ω-functionalized fatty acids with E. coli JW5020-1 Kan^{S} pBT10_alkL/pJ294[alaDH_Bs_TAcv(ct_Ptac-CpFATB2]. The concentrations of lauric acid and ω-carboxylauric acid, ω-hydroxylauric acid and ω-aminolauric acid after 60 hours fermentation are stated:

### Example 4

### Production of an E. coli Expression Vector for the Genes fadD from Escherichia coli and atfA from Acinetobacter sp. ADP1

For the production of the E. coli expression vector for the genes fadD (SEQ ID No. 21) from Escherichia coli (coding for enzyme Eᵥ) and atfA with terminator (SEQ ID No. 22) from Acinetobacter sp. ADP1 (coding for enzyme Eᵥ) under control of a tac promoter, these genes were amplified from chromosomal DNA of E. coli W3110 and Acinetobacter calcoaceticus ADP1 respectively by PCR with incorporation of homologous regions for the recombination cloning.

The synthetic tac promoter (SEQ ID No. 23) was amplified with ribosome binding site from a pJ294 derivative (DNA 2.0; Menlo Park, Calif., USA) with incorporation of homologous regions.

The preparation of the chromosomal DNA from E. coli W3110 and Acinetobacter calcoaceticus ADP1 was effected by means of DNeasy Blood & Tissue Kit (Qiagen, Hilden) according to the manufacturer's instructions. In the amplification of the genes fadD from E. coli and atfA from Acinetobacter sp. ADP1 with chromosomal DNA of E. coli W3110 and Acinetobacter calcoaceticus ADP1 respectively as matrix, and the amplification of the synthetic promoter P_{tac} from a pJ294 derivative the following oligonucleotides were used:

Ptac:

| | |
|---|---|
| 11- | 5′-TTATGCGACTCCTGCGTTTAGGGAAAGAGCATTTG-3′ |
| 001_fw: | (SEQ ID No. 24) |
| Ptac-rv: | 5′-GTTAACATATGTTTTACCTCCTGTTAAACAAA-3′ |
| | (SEQ ID No. 25) |

fadD [E. coli]:

| | |
|---|---|
| fad D- | 5′-TAAAACATATGTTAACGGCATGTATATCATTT-3′ |
| fw: | (SEQ ID No. 26) |
| fadD-rv: | 5′-TCTCCTCAGACTTAACGCTCAGGCTTTATTGT-3′ |
| | (SEQ ID No. 27) |

atfA [Acinetobacter sp. ADP1]:

| | |
|---|---|
| atfA-fw: | 5′-GTTAAGTCTGAGGAGATCCACGCTATGCGCCC-3′ |
| | (SEQ ID No. 28) |
| 11- | 5′-CAATTGAGATCTGCCACGACTGCAATGGTTCATC-3′ |
| 002_rv: | (SEQ ID No. 29) |

The following parameters were used for the PCR: 1×: initial denaturation, 103° C., 3:00 mins; 35×: denaturation, 98° C., 0:10 mins, annealing, 65° C., 0:15 min; elongation, 72° C., 0:45 mins; 1×: terminal elongation, 72° C., 10 mins. For the amplification, the Phusion™ High-Fidelity Master Mix from New England Biolabs (Frankfurt) was used according to the manufacturer's recommendations. 50 μl of each PCR reaction were then separated on a 1% TAE agarose gel. The PCR, agarose gel electrophoresis, ethidium bromide staining of the DNA and determination of the PCR fragment sizes were performed in the manner known to those skilled in the art.

In all cases, PCR fragments of the expected size could be amplified. These were: for the promoter region P_{tac} 607 bp, for fadD 1778 bp and for atfA 1540 bp.

For the isolation of the DNA from an agarose gel, the target DNA was isolated from the gel with a scalpel and purified with the Quick Gel Extraction Kit from Qiagen (Hilden) according to the manufacturer's instructions. The purified PCR products were recombined with the EcoNI/NdeI-cleaved vector pCDFDuet™-1 (71340-3, Merck, Darmstadt) by means of in vitro cloning with use of the Geneart Seamless Cloning and Assembly Kit from Invitrogen (Darmstadt). The use corresponded to the manufacturer's recommendations. pCDFDuet-1 is an E. coli vector which mediates a spectinomycin/streptomycin resistance in the organism and carries a CoIDF13 replication origin. The transformation of chemically competent E. coli DH5α cells (New England Biolabs, Frankfurt) was effected in a manner known to those skilled in the art.

The correctness of the plasmid was checked by a restriction analysis with XbaI. The authenticity of the inserted fragments was checked by DNA sequencing. The finished E. coli expression vector was designated as pCDF[fadD-atfA] (SEQ ID No. 30).

### Example 5

### Production of Methyl Aminolaurate and Ethyl Aminolaurate by an E. coli Strain with Deletion of the Gene fadE and Expression Vectors for the Genes fatB2 from Cuphea palustris, Ald from Bacillus subtilis, and Cv_{—}2025 from Chromobacterium violaceum in Combination with an Expression Vector for the Genes alkB, alkG, alkT and alkL from the Alk Operon of Pseudomonas putida and an Expression Vector for the Genes fadD from Escherichia coli and atfA from Acinetobacter sp. ADP1

To create an E. coli strain with expression vectors for the genes fatB2 from Cuphea palustris (coding for enzyme Eᵢ), a/d from Bacillus subtilis (coding for enzyme E₃) and Cv_{—}2025 from Chromobacterium violaceum (coding for enzyme E₂) in combination with an expression vector for the genes alkB (coding for enzyme E_{1b}), alkG, alkT (coding for auxiliary enzymes to enzyme E_{1b}) and alkL (coding for alkL gene product) from the alk operon of Pseudomonas putida GPo1 and an expression vector for the genes fadD from Escherichia coli (coding for enzyme Eᵥᵢ) and atfA from Acinetobacter sp. ADP1 (coding for enzyme Eᵥ), electrocompetent cells of E. coli JW5020-1 Kan^{S} are produced. This takes place in a manner known to those skilled in the art. E. coli JW5020-1 Kan^{S} is a derivative of E. coli JW5020-1 (CGSC, The coli genetic stock center, Yale University, New Haven, USA), and this in turn is an E. coli BW25113 derivative which carries a deletion of the gene fadE (coding for enzyme E_{b}). The gene fadE was replaced by a kanamycin cassette. Before the provision of the strain with the expression vectors by means of a helper plasmid, which codes for the Flp recombinase, this was removed in a manner known to those skilled in the art (see Datsenko K. A. and Wanner B. L. (2000) PNAS 97(12):6640-6645), resulting in the strain E. coli JW5020-1 Kan^{S}. E. coli JW5020-1 Kan^{S} and E. coli W3110 ΔfadE (construction described in Example 8) are sequentially transformed with the plasmids pBT10_alkL, pCDF[fadD-atfA] and pJ294[alaDH_B.s._TA_C.v.(Ct)_Ptac-CpFATB2] and plated out onto LB agar plates containing kanamycin (50 μg/ml), spectinomycin (100 μg/ml) and ampicillin (100 μg/ml). Transformants are checked for the presence of the correct plasmids by plasmid preparation and analytical restriction analysis. In this manner, the following strains are constructed: E. coli JW5020-1 Kan^{S} pBT10_alkL/pCDF[fadD-atfA]/pJ294[alaDH_B.s._TA_C.v.(Ct)_Ptac-CpFATB2] and E. coli W3110 ΔfadE pBT10_alkL/pCDF[fadD-affA]/pJ294[alaDH_B.s._TA_C.v.(Ct)_Ptac-CpFATB2].

The strain is subjected to fed-batch fermentation in order to analyse its capacity for the production of methyl aminolaurate and ethyl aminolaurate from glucose. The strain to be tested is firstly grown from a glycerine culture as a preculture in M9 medium containing kanamycin (50 μg/ml), spectinomycin (100 μg/ml) and ampicillin (100 μg/ml) at 37° C. overnight. The medium, consisting of 38 mM disodium hydrogen phosphate dihydrate, 22 mM potassium dihydrogen phosphate, 8.6 mM sodium chloride, 37 mM ammonium chloride, 1.5% (w/v) glucose, 2 mM magnesium sulphate heptahydrate (all substances from Merck, Darmstadt) and 0.5% (v/v) trace element solution, is adjusted to a pH of 7.4 with 25% ammonium hydroxide solution. The trace element solution added, consisting of 9.7 mM manganese(II) chloride tetrahydrate, 6.5 mM zinc sulphate heptahydrate, 2.5 mM sodium EDTA (Titriplex III), 4.9 mM boric acid, 1 mM sodium molybdate dihydrate, 32 mM calcium chloride dihydrate, 64 mM iron(II) sulphate heptahydrate and 0.9 mM copper(II) chloride dihydrate dissolved in 37% hydrochloric acid (all substances from Merck, Darmstadt), is sterile-filtered before addition to the M9 medium. The fermenter is inoculated with the preculture such that an optical density of 0.2 is reached. The culturing is effected at a pH of 6.8, regulated with 25% aqueous ammonia and 0.5 M sulphuric acid, an oxygen partial pressure of 20%, regulated via the stirrer speed and the air feed, and a temperature of 37° C. The glucose feed is effected after consumption of the glucose present in the medium, at a feed rate of 5 g/l/hr based on the initial volume. At the start of the glucose feed, the temperature is adjusted to 30° C. Gene expression is induced 2 hours after the start of the glucose feed by addition of 1 mM isopropyl-β-D-thiogalactopyranoside and 0.025% dicyclopropyl ketone. Simultaneously with the induction, 2% (v/v) methanol or 2% (v/v) ethanol are added as methyl group donor or ethyl group donor for the fatty acid esterification.

The strain is cultured for at least a further 48 hours under constant conditions. During the culturing, 1 ml samples are withdrawn and the concentration of fatty acid methyl esters, fatty acid ethyl esters, ω-functionalized fatty acid methyl esters and ω-functionalized fatty acid ethyl esters quantified by the method described in Example 2. It is shown that the strains E. coli JW5020-1 Kan^{S} pBT10_alkL/pCDF[fadD-atfA]/pJ294[alaDH_B.s._TA_C.v.(Ct)_Ptac-CpFATB2] and E. coli W3110 ΔfadE pBT10_alkL/pCDF[fadD-atfA]/pJ294[alaDH_B.s._TA_C.v.(Ct)_Ptac-CpFATB2] are capable of forming methyl laurate, methyl ω-hydroxylaurate, methyl ω-oxolaurate, methyl ω-aminolaurate and methyl ω-carboxylaurate with addition of 2% (v/v) methanol and ethyl laurate, ethyl ω-hydroxylaurate, ethyl ω-oxolaurate, ethyl ω-aminolaurate and ethyl ω-carboxylaurate with addition of 2% (v/v) ethanol respectively.

### Example 6

### Production of Expression Vectors for the Genes synUcTE from Umbellularia californica, Ald from Bacillus subtilis and Cv_{—}2025 from Chromobacterium violaceum

For the production of an E. coli expression vector for the genes synUcTE (SEQ ID No. 31) from Umbellularia californica (coding for an enzyme Eᵢ), aid (SEQ ID No. 33) from Bacillus subtilis (coding for an enzyme E₃) and Cv_{—}2025 (SEQ ID No. 35) from Chromobacterium violaceum (coding for an enzyme E₂), the gene synUcTE was codon-optimized for expression in Escherichia coli and synthesized together with a tac promoter (SEQ ID No. 37). During the synthesis, a cleavage site upstream of the promoter and a cleavage site downstream of the terminator were introduced. The synthesized DNA fragment P_{tac} synUcTE was digested with the restriction endonucleases BamHI and NotI and ligated into the correspondingly cleaved vector pJ294_alaDH_B.s._TA_C.v.(Ct) (SEQ ID No. 17) and the vector pJ294 (DNA2.0 Inc., Menlo Park, Calif., USA). The finished vectors were designated as pJ294[alaDH_B.s._TA_C.v.(Ct)_Ptac-synUcTE] (SEQ ID No. 38) and pJ294[Ptac-synUcTE] (SEQ ID No. 39).

### Example 7

### Production of Expression Vectors for the Genes fadD from Escherichia coli and Wax-dgaT from Acinetobacter sp. ADP1 and atfA1 from Alcanivorax borkumensis

For the production of expression vectors for the genes fadD (SEQ ID No. 21) from Escherichia coli and wax-dgaT (SEQ ID No. 42) (coding for an enzyme Eᵥ) from Acinetobacter sp. ADP1 and atfA1 (SEQ ID No. 44) (coding for an enzyme Eᵥ) from Alcanivorax borkumensis SK2 respectively, the genes wax-dgaT and atfA1 were codon-optimized for expression in Escherichia coli and synthesized in combination with the Gen fadD from E. coli (coding for an enzyme Eᵥᵢ). The synthesized DNA fragments wax-dgaT_AsADP1-fadD_Ec (SEQ ID No. 46) and atfA1_Ab-fadD_Ec (SEQ ID No. 47) were amplified with incorporation of homologous regions for recombination cloning.

For the amplification of the fragment wax-dgaT_AsADP1-fadD_Ec the following oligonucleotides were used:

| wax-dgaT_H1_fw: | 5′-ACAGGAGGTAAAACATATGCGTCCTCTGCACCCG-3′ |
|---|---|
| | (SEQ ID No. 51) |
| fadD_H2_rv: | 5′-GTTTCTTTACCAGACTCGAGATTGTTTTCTCTTTAGTGGGCGTC-3′ |
| | (SEQ ID No. 52) |
| For the amplification of the fragment atfALAb-fadD Ec the following | |
| oligonucleotides were used: | |
| affA_Abfw_kurz: | 5′-ACAGGAGGTAAAACATATGAAAGCGCTGTCCC-3′ |
| | (SEQ ID No. 53) |
| fadD_H2_rv_N: | 5′-GTTTCTTTACCAGACTCGAGATIGTTTTCTCTTTAGTGGGC-3′ |
| | (SEQ ID No. 54) |

The following parameters were used for the PCR: 1×: initial denaturation, 98° C., 0:30 mins; 35×: denaturation, 98° C., 0:10 min, annealing, 70° C., 0:20 mins; elongation, 72° C., 1 min; 1×: terminal elongation, 72° C., 10 min. For the amplification, the Phusion™ High-Fidelity Master Mix from New England Biolabs (Frankfurt) was used according to the manufacturer's recommendations. 50 μl of each PCR reaction were then separated on a 1% TAE agarose gel. The PCR, agarose gel electrophoresis, ethidium bromide staining of the DNA and determination of the PCR fragment sizes were performed in the manner known to those skilled in the art. In both cases, PCR fragments of the expected size could be amplified. These were 3192 base pairs for wax-dgaT_AsADP1-fadD_Ec and 3189 base pairs for atfA1_Ab-fadD_Ec. For the isolation of the DNA from the agarose gel, the target DNA was cut out from the gel with a scalpel and purified with the QiaQuick Gel extraction Kit according to the manufacturer's instructions (Qiagen, Hilden). The purified PCR products were cloned into a NdeI- and XhoI-cleaved pCDF derivative, which already contains a synthetic tac promoter (SEQ ID No. 50), by means of recombination with use of the Geneart® Seamless Cloning and Assembly Kit according to the manufacturer's instructions (Life Technologies, Carlsbad, Calif., USA). The transformation of chemically competent E. coli DH5α (New England Biolabs, Frankfurt) was effected in a manner known to those skilled in the art. The correct insertion of the target genes was checked by restriction analysis and the authenticity of the incorporated genes confirmed by DNA sequencing. The resulting expression vectors were designated as pCDF[wax-dgaT_AsADP1(co_Ec)-fadD_Ec] (SEQ ID No. 48) and pCDF[atfA1_Ab(co_Ec)-fadD_Ec] (SEQ ID No. 49).

### Example 8

### Production of Methyl Aminolaurate by an E. coli Strain with Deletion in the Gene fadE and Expression Vectors for the Genes synUcTE from Umbellularia californica, Ald from Bacillus subtilis, and Cv_{—}2025 from Chromobacterium violaceum in Combination with an Expression Vector for the Genes alkB, alkG, alkT and alkL from the alk operon of Pseudomonas putida and an Expression Vector for the Genes fadD from Escherichia coli and Wax-dgaT from Acinetobacter sp. ADP1 or atfA1 from Alcanivorax borkumensis

Firstly, an E. coli strain with deletion in the gene fadE (SEQ ID No. 40) was constructed. For the production of the gene deletion, a plasmid was constructed which carries the DNA sequence ΔfadE (SEQ ID No. 55). This sequence was synthesized and consists of homologous regions 500 base pairs upstream and downstream from the fadE gene and the recognition sequence for the restriction endonuclease NotI at the 5′ and 3′ end. The sequence ΔfadE was digested with the restriction endonuclease NotI and ligated into the correspondingly cleaved vector pKO3. The strain E. coli W3110 ΔfadE was constructed by means of the pKO3-ΔfadE construct (SEQ ID No. 56) by methods known to those skilled in the art (see Link A J, Phillips D, Church G M. J. Bacteriol. 1997. 179(20)). The DNA sequence of fadE after deletion is reproduced in SEQ ID No. 57.

To create an E. coli strain with expression vectors for the genes synUcTE from Umbellularia californica, ald from Bacillus subtilis, and Cv_{—}2025 from Chromobacterium violaceum in combination with an expression vector for the genes alkB (enzyme E₁ₐ), alkG, alkT (auxiliary enzymes to enzyme E_{1b}) and alkL (coding for alkL gene product) from the alk operon of Pseudomonas putida GPo1 and an expression vector for the genes fadD from Escherichia coli and wax-dgaT from Acinetobacter sp. ADP1 or atfA1 from Alcanivorax borkumensis SK2, electrocompetent cells of E. coli W3110 ΔfadE were produced. This took place in a manner known to those skilled in the art. E. coli W3110 ΔfadE was sequentially transformed with the plasmids pBT10_alkL (sequence and production: compare Example 1 of PCT/EP2011/053834 and the Seq ID No. 8 listed there), pJ294[alaDH_B.s._TA_C.v.(Ct)_Ptac-synUcTE] (SEQ ID No. 38) and pCDF[wax-dgaT_AsADP1(co_Ec)-fadD_Ec] (SEQ ID No. 48) and pCDF[atfA1_Ab(co_Ec)-fadD_Ec] (SEQ ID No. 49) respectively and plated out onto LB agar plates containing kanamycin (50 μg/ml), ampicillin (100 μg/ml) and spectinomycin (100 μg/ml). Transformants were checked for the presence of the correct plasmids by plasmid preparation and analytical restriction analysis. In this manner the following strains were constructed:

E. coli W3110 ΔfadE pBT10_alkL/pJ294[alaDH_B.s._TA_C.v.(Ct)_Ptac-synUcTE]/pCDF[wax-dgaT_AsADP1(co_Ec)-fadD_Ec]
E. coli W3110 ΔfadE pBT10_alkL/pJ294[alaDH_B.s._TA_C.v.(Ct)_Ptac-synUcTE]/pCDF[atfA1_Ab(co_Ec)-fadD_Ec]

The strains were subjected to a fed-batch fermentation in order to analyse their capacity for the production of methyl hydroxylaurate, methyl oxolaurate, methyl carboxylaurate and methyl aminolaurate from glucose. This was performed with an 8-fold parallel fermentation system from DASGIP.

For the fermentation, 1 L reactors were used, which were equipped with overhead stirrers and impeller turbines. For process monitoring, pH and pO₂ were measured online. OTR/CTR measurements were used inter alia for estimation of the metabolic selectivity and fitness of the cells.

The pH probes were calibrated by means of a two-point calibration with standard solutions of pH 4.0 and pH 7.0 according to the technical manual of DASGIP. The reactors were equipped with the necessary sensors and connections according to the technical manual and the stirrer shaft mounted. They were filled with 300 mL water and autoclaved for 20 mins at 121° C. to ensure sterility. Next, the pO2 probes were polarized overnight after connection to the measurement amplifier. Next, the water was removed under the clean bench, and replaced by high cell density medium consisting of (NH₄)₂SO4 1.76 g/L, K₂HPO₄ 19.08 g/L, KH₂PO₄ 12.5 g/L, yeast extract 6.66 g/L, trisodium citrate dihydrate 11.2 g, 17mL/L of a separately autoclaved 1% ammonium iron citrate solution, and 5 mL/L of separately autoclaved trace element stock solution (consisting of HCl (37%) 36.50 g/L, MnCl₂*4H₂O 1.91 g/L, ZnSO₄*7H₂O 1.87 g/L, ethylenediaminetetraacetic acid dihydrate 0.84 g/L, H₃BO₃ 0.30 g/L. Na₂MoO₄*2H₂O 0.25 g/L, CaCl₂*2H₂O 4.70 g/L, FeSO₄*7H₂O 17.80 g/L, CuCl₂*2H₂O 0.15 g/L) with 15 g/L glucose as carbon source (added by metering in 30 mL/L of a separately autoclaved feed solution consisting of 500 g/L glucose, 1% (w/v) MgSO₄*7H₂O and 2.2% (w/v) NH₄Cl) containing 100 mg/L ampicillin, 50 mg/L kanamycin and 100 mg/L spectinomycin.

Below, the pO2 probes were calibrated with a one-point calibration (stirrer: 600 rpm/gassing: 10 sL/hr air) and the feed-, correction agent and induction agent lines cleaned by means of Cleaning-in-Place according to the technical manual. For this, the tubes were flushed with 70% ethanol, then with 1 M NaOH, then with sterile demineralized water and finally filled with the respective media.

The strains W3110 ΔfadE pJ294[alaDH_Bs/TAcv(ct)]{Ptac}[synUcTE] pBT10_alkL pCDF[atfA_AsADP1(co_Ec)/fadD] and W3110 ΔfadE

pJ294[alaDH_Bs/TAcv(ct)]{Ptac}[synUcTE] pBT10_alkL pCDF[atfA1_Ab(co_Ec)/fadD] were firstly grown from a cryoculture in LB medium (25 mL in a 100 mL baffle flask) containing 100 mg/L ampicillin, 50 mg/L kanamycin and 100 mg/L spectinomycin overnight at 37° C. and 200 rpm for ca. 18 hrs. Next, 2 mL of this culture were transferred for a second preculture stage into 25 mL of high cell density medium consisting of (NH₄)₂SO₄ 1.76 g/L, K₂HPO₄ 19.08 g/L, KH₂PO₄ 12.5 g/L, yeast extract 6.66 g/L, trisodium citrate dihydrate 11.2 g, 17 mL/L of a separately autoclaved 1% ammonium iron citrate solution, and 5 mL/L of separately autoclaved trace element stock solution (consisting of HCl (37%) 36.50 g/L, MnCl₂*4H₂O 1.91 g/L, ZnSO₄*7H₂O 1.87 g/L, ethylenediaminetetraacetic acid dihydrate 0.84 g/L, H₃BO₃ 0.30 g/L. Na₂MoO₄*2H₂O 0.25 g/L, CaCl₂*2H₂O 4.70 g/L, FeSO₄*7H₂O 17.80 g/L, CuCl₂*2H₂O 0.15 g/L) with 15 g/L glucose as carbon source (added by metering in 30 mL/L of a separately autoclaved feed solution consisting of 500 g/L glucose, 1% (w/v) MgSO₄*7H₂O and 2.2% (w/v) NH₄Cl) containing the antibiotics already described in a 100 mL shaker flask and again incubated at 37° C./200 rpm for a further 6 hrs.

In order to inoculate the reactors with an optical density of 0.1, the OD of the second preculture stage was measured and the quantity of culture required for the inoculation calculated. The required quantity of culture was added to the thermostatted and aerated reactor through a septum by means of a 5 mL syringe.

The following standard programme was used:

| | |
|---|---|
| DO regulator | pH regulator |

| | | | |
|---|---|---|---|
| Preset | 0% | Preset | 0 mL/hr |
| P | 0.1 | P | 5 |
| Ti | 300 secs | Ti | 200 secs |
| Min | 0% | Min | 0 mL/hr |
| Max | 100% | Max | 40 mL/hr |

| N | | | XO2 (gas | | | F (gas | | |
|---|---|---|---|---|---|---|---|---|
| (Rotation) | from | to | mixture) | from | to | flow) | from | to |
| Growth | 0% | 30% | Growth | 0% | 100% | Growth | 15% | 80% |
| and | 400 rpm | 1500 rpm | and | 21% | 21% | and | 6 sL/hr | 72 sL/hr |
| biotransformation | | | biotransformation | | | biotransformation | | |

| Script | |
|---|---|
| Trigger | 31% DO (1/60 hr) |
| exactly | |
| Induction | 2 hrs after feed start |
| IPTG | |
| Feed trigger | 50% DO |
| Feed rate | 3 [mL/hr] |

The pH was regulated at pH 6.8 with 12.5% ammonia solution. During growth and biotransformation, the dissolved oxygen (pO₂ or DO) in the culture was regulated to at least 30% via stirrer revolution rate and gassing rate. After inoculation, the DO fell from 100% to this 30%, where it was maintained stable for the remainder of the fermentation.

The fermentation was performed as fed-batch, wherein as entry to the fed phase, with 5 g/Lhr glucose feed consisting of 500 g/L glucose, 1% (w/v) MgSO₄*7H₂O and 2.2% (w/v) NH₄Cl, the feed start was triggered via the DO peak indicating the end of the batch phase. At feed start, the temperature was also lowered from 37° C. to 30° C.

At this time, 24 mL of oleic acid, an oleic acid/hexadecane (1:1 (w/w)) or 2-hexyldecanoic acid/hexadecane mixture (1:1 (w/w)) were added to the reactors. 2 hrs after feed start, the expression of the genes fadD from Escherichia coli and atfA from Acinetobacter sp. ADP1 or atfA1 from Alcanivorax borkumensis SK2 was induced by the automatic addition of a prepared IPTG solution for 1 mM final concentration in the reactor. This IPTG solution also contained 3 mL methanol, which is required for the esterification of the fatty acids produced. The induction of the alkBGT genes was effected by the manual addition of 0.025% (v/v) DCPK 10 hrs after feed start.

During the culturing, samples were withdrawn and the concentration of fatty acids and ω-functionalized fatty acids quantified with the method described below.

For the sampling, 2 mL of fermentation broth were withdrawn from the vessel and a portion thereof diluted 1/10 in an acetonitrile-formic acid mixture (80% (v/v) acetonitrile in water; 0.1% (v/v) formic acid).

The quantification of methyl laurate LSME, methyl ω-hydroxylaurate HLSME, methyl ω-oxolaurate OLSME, methyl ω-aminolaurate ALSME and methyl ω-carboxylaurate DDSME, ω-aminolauric acid ALS, ω-carboxylauric acid DDS, lauric acid LS, ω-hydroxylauric acid HLS and ω-oxolauric acid OLS in the fermentation samples was effected by means of LC-ESI/MS² on the basis of an external calibration for all analytes (0.1-50 mg/L) and with use of the internal standards aminoundecanoic acid (AUD), d4-ALSME, 13C-DDSME and d3-LSME.

For this, the following instruments were used:
HPLC unit 1260 (Agilent; Böblingen) with autosampler (G1367E), binary pump (G1312B) and column oven (G1316A)
Mass spectrometer TripelQuad 6410 (Agilent; Boblingen) with ESI source
HPLC column: Kinetex C18, 100×2.1 mm, particle size: 2.6 μm, pore size 100 Å (Phenomenex; Aschaffenburg)
Precolumn: KrudKatcher Ultra HPLC in-line filter; 0.5 μm filter depth and 0.004 mm internal diameter (Phenomenex; Aschaffenburg)

The samples were prepared by pipetting 1900 μL of solvent (80% (v/v) ACN, 20% bidest. H₂O (v/v), +0.1% formic acid) and 100 μL of sample into a 2 mL reaction vessel. The mixture was vortexed for ca. 10 seconds and then centrifuged at ca. 13,000 rpm for 5 mins. The clear supernatant was withdrawn with a pipette and analysed after appropriate dilution with diluent (80% (v/v) ACN, 20% bidest. H₂O (v/v), +0.1% formic acid). 100 μL of ISTD were pipetted into each 900 μL sample (10 μL with a sample volume of 90 μL).

The HPLC separation was effected with the aforementioned column and precolumn. The injection volume was 0.7 μL, the column temperature 50° C. and the flow rate 0.6 mL/min. The mobile phase consisted of eluent A (0.1% (v/v) aqueous formic acid) and eluent B (acetonitrile with 0.1% (v/v) formic acid). The following gradient profile was used

| Time [mins] | Eluent A [%] | Eluent B [%] |
|---|---|---|
| 0 | 77 | 23 |
| 0.3 | 77 | 23 |
| 0.4 | 40 | 60 |
| 2.5 | 40 | 60 |
| 2.6 | 2 | 98 |
| 5.5 | 2 | 98 |
| 5.6 | 77 | 23 |
| 9 | 77 | 23 |

The ESI-MS² analysis was effected in positive mode with the following ESI source parameters:
Gas temperature 280° C.
Gas flow rate 11 L/min
Nebulizer pressure 50 psi
Capillary voltage 4000 V

The detection and quantification of the individual compounds was effected with the following parameters, wherein in each case one product ion was used as Qualifier and one as Quantifier.

| | Precursor Ion | Product Ion | Dwell | Collision |
|---|---|---|---|---|
| Analyte | [m/z] | [m/z] | time [ms] | energy [eV] |
| DDSME | 245.2 | 167.1 | 25 | 6 |
| DDSME | 245.2 | 149.1 | 50 | 8 |
| HLSME | 231.3 | 181.2 | 15 | 2 |
| HLSME | 231.3 | 163.2 | 25 | 5 |
| DDS | 231.2 | 213.2 | 50 | 0 |
| DDS | 231.2 | 149.1 | 25 | 9 |
| ALSME | 230.3 | 198.1 | 25 | 10 |
| ALSME | 230.3 | 163.2 | 15 | 10 |
| OLSME | 229.2 | 197.2 | 50 | 0 |
| OLSME | 229.2 | 161.1 | 25 | 5 |
| HLS | 217.2 | 181.2 | 35 | 0 |
| HLS | 217.2 | 163.1 | 20 | 4 |
| OLS | 215.2 | 161.2 | 25 | 0 |
| OLS | 215.2 | 95.2 | 60 | 13 |

It was shown that the strains E. coli W3110 ΔfadE pBT10_alkL/pJ294[alaDH_B.s._TA_C.v.(Ct)_Ptac-synUcTE]/pCDF[wax-dgaT_AsADP1(co_Ec)-fadD_Ec] and E. coli W3110 ΔfadE pBT10_alkL/pJ294[alaDH_B.s._TA_C.v.(Ct)_Ptac-synUcTE]/pCDF[atfA1_Ab(co_Ec)-fadD_Ec] with addition of 1% (v/v) methanol are capable of forming methyl laurate, methyl ω-hydroxylaurate, methyl ω-oxolaurate, methyl ω-aminolaurate and methyl ω-carboxylaurate. In addition it could be shown that under these conditions both strains also form lauric acid, ω-hydroxylauric acid, ω-oxolauric acid, ω-aminolauric acid and ω-carboxylauric acid.

Production of ω-functionalized fatty acid methyl esters. The concentrations of methyl laurate, methyl ω-carboxylaurate, methyl ω-hydroxylaurate, methyl ω-oxolaurate and methyl ω-aminolaurate after 38.33 hours fermentation time are stated.

| | | C_{methyl laurate} | C_{methyl ω-carboxylaurate} |
|---|---|---|---|
| Strain | Added organic phase | [mg/L] | [mg/L] |
| E. coli W3110 ΔfadE pBT10_alkL/ | Oleic acid | 28.4 | 1.8 |
| pJ294[alaDH_B.s._TA_C.v.(ct)_Ptac- | | | |
| synUcTE]/pCDF[wax- | | | |
| dgaT_AsADP1(co_Ec)-fadD_Ec] | | | |
| E. coli W3110 ΔfadE pBT10_alkL/ | Oleic acid | 20.2 | 0.4 |
| pJ294[alaDH_B.s._TA_C.v.(ct)_Ptac- | | | |
| synUcTE]/pCDF[atfA1_Ab(co_Ec)-fadD_Ec] | | | |
| E. coli W3110 ΔfadE pBT10_alkL/ | Oleic acid/ | 13.2 | 1.4 |
| pJ294[alaDH_B.s._TA_C.v.(ct)_Ptac- | hexadecane | | |
| synUcTE]/pCDF[wax- | | | |
| dgaT_AsADP1(co_Ec)-fadD_Ec] | | | |
| E. coli W3110 ΔfadE pBT10_alkL/ | 2-hexyl- | 7.5 | 0.9 |
| pJ294[alaDH_B.s._TA_C.v.(ct)_Ptac- | decanoic | | |
| synUcTE]/pCDF[wax- | acid/ | | |
| dgaT_AsADP1(co_Ec)-fadD_Ec] | hexadecane | | |

| | C_{methyl ω-hydroxylaurate} | C_{methyl ω-oxolaurate} | C_{methyl ω-aminolaurate} |
|---|---|---|---|
| Strain | [mg/L] | [mg/L] | [mg/L] |
| E. coli W3110 ΔfadE pBT10_alkL/ | 4.6 | 2.0 | 4.0 |
| pJ294[alaDH_B.s._TA_C.v.(ct)_Ptac- | | | |
| synUcTE]/pCDF[wax- | | | |
| dgaT_AsADP1(co_Ec)-fadD_Ec] | | | |
| E. coli W3110 ΔfadE pBT10_alkL/ | 0.8 | 1.2 | 2.2 |
| pJ294[alaDH_B.s._TA_C.v.(ct)_Ptac- | | | |
| synUcTE]/pCDF[atfA1_Ab(co_Ec)-fadD_Ec] | | | |
| E. coli W3110 ΔfadE pBT10_alkL/ | 2.1 | 1.3 | 2.6 |
| pJ294[alaDH_B.s._TA_C.v.(ct)_Ptac- | | | |
| synUcTE]/pCDF[wax- | | | |
| dgaT_AsADP1(co_Ec)-fadD_Ec] | | | |
| E. coli W3110 ΔfadE pBT10_alkL/ | 1.7 | 0.1 | 40.6 |
| pJ294[alaDH_B.s._TA_C.v.(ct)_Ptac- | | | |
| synUcTE]/pCDF[wax- | | | |
| dgaT_AsADP1(co_Ec)-fadD_Ec] | | | |

### Example 9

### Production of Methyl Aminolaurate by an E. coli Strain with Deletion of the Gene fadE and Expression Vectors for the Genes synUcTE from Umbellularia californica, Ald from Bacillus subtilis, and Cv_{—}2025 from Chromobacterium violaceum in Combination with an Expression Vector for the Genes alkB, alkG and alkT from the alk Operon of Pseudomonas putida and an Expression Vector for the Genes fadD from Escherichia coli and Wax-dgaT from Acinetobacter sp. ADP1 or atfA1 from Alcanivorax borkumensis

To create an E. coli strain with expression vectors for the genes synUcTE from Umbellularia californica, ald from Bacillus subtilis, and Cv_{—}2025 from Chromobacterium violaceum in combination with an expression vector for the genes alkB, alkG and alkT from the alk operon of Pseudomonas putida GPo1 and an expression vector for the genes fadD from Escherichia coli and wax-dgaT from Acinetobacter sp. ADP1 or atfA1 from Alcanivorax borkumensis SK2, electrocompetent cells of E. coli W3110 ΔfadE (construction described in Example 8) are produced. This takes place in a manner known to those skilled in the art. E. coli W3110 ΔfadE is sequentially transformed with the plasmids pBT10 (construction described in practical example B.2 of PCT/EP2008/067447), pJ294[alaDH_B.s._TA_C.v.(Ct)_Ptac-synUcTE] (SEQ ID No. 5) and pCDF[wax-dgaT_AsADP1(co_Ec)-fadD_Ec] (SEQ ID No. 16) and pCDF[atfA1_Ab(co_Ec)-fadD_Ec] (SEQ ID No. 17) respectively and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis. In this manner, the following strains are constructed:
E. coli W3110 ΔfadE pBT10/pJ294[alaDH_B.s._TA_C.v.(Ct)_Ptac-synUcTE]/pCDF[wax-dgaT_AsADP1(co_Ec)-fadD_Ec]
E. coli W3110 ΔfadE pBT10/pJ294[alaDH_B.s._TA_C.v.(Ct)_Ptac-synUcTE]/pCDF[atfA1_Ab(co_Ec)-fadD_Ec]

The strains are subjected to a fed-batch fermentation in order to analyse their capacity for the production of methyl hydroxylaurate, methyl oxolaurate, methyl carboxylaurate and methyl aminolaurate from glucose. This is performed with an 8-fold parallel fermentation system from DASGIP, as described in Example 8.

It is shown that the strains E. coli W3110 ΔfadE pBT10/pJ294[alaDH_B.s._TA_C.v.(Ct)_Ptac-synUcTE]/pCDF[wax-dgaT_AsADP1(co_Ec)-fadD_Ec] and E. coli W3110 ΔfadE pBT10/pJ294[alaDH_B.s._TA_C.v.(Ct)_Ptac-synUcTE]/pCDF[atfA1_Ab(co_Ec)-fadD_Ec] with addition of 1% (v/v) methanol are capable of forming lauric acid, methyl laurate, methyl ω-hydroxylaurate, methyl ω-oxolaurate, methyl ω-aminolaurate and methyl ω-carboxylaurate.

### Example 10

### Production of Methyl Aminohexanoate, Methyl Aminooctanoate, Methyl Aminodecanoate, Methyl Aminotetradecanoate and Methyl Amino-9-Hexadecenoate by E. coli Strains without Deletion of an fad Gene and with Deletion of One of the Genes fadA, fadB, fadD, fadE or fadL and Expression Vectors for the Genes synUcTE from Umbellularia californica, ChFATB2 from Cuphea hookeriana, CnFATB3 from Cocos nucifera or CPF 2954 from Clostridium perfringens and Ald from Bacillus subtilis, Cv_{—}2025 from Chromobacterium violaceum in Combination with an Expression Vector for the Genes alkB, alkG and alkT from the alk Operon of Pseudomonas putida and an Expression Vector for the Genes fadD from Escherichia coli and Wax-dgaT from Acinetobacter sp. ADP1 or atfA1 from Alcanivorax borkumensis

To create E. coli strains with expression vectors for the genes synUcTE from Umbellularia californica, ChFATB2 from Cuphea hookeriana (SEQ ID No. 58), CnFATB3 from Cocos nucifera (SEQ ID No. 60) or CPF_{—}2954 from Clostridium perfringens (SEQ ID No. 62) and ald from Bacillus subtilis, and Cv_{—}2025 from Chromobacterium violaceum in combination with an expression vector for the genes alkB, alkG and alkT from the alk operon of Pseudomonas putida GPo1 and an expression vector for the gene fadD from Escherichia coli and wax-dgaT from Acinetobacter sp. ADP1 or atfA1 from Alcanivorax borkumensis SK2, electrocompetent cells of E. coli BW25113, JW5578-1 Kan^{S}, JW3822-1 Kan^{S}, JW1794-1 Kan^{S}, JW5020-1 Kan^{S} and JW2341-1 Kan^{S} are produced. This takes place in a manner known to those skilled in the art.

E. coli JW5578-1 Kan^{S}, JW3822-1 Kan^{S}, JW1794-1 Kan^{S}, JW5020-1 Kan^{S} and JW2341-1 Kan^{S} are derivatives of E. coli E. coli JW5578-1, JW3822-1, JW1794-1, JW5020-1 and JW2341-1 (CGSC, The coli genetic stock center, Yale University, New Haven, USA), and these in turn are E. coli BW25113 derivatives which carry a deletion of the genes fadA (SEQ ID No. 64; coding for enzyme E_{f}), fadB (SEQ ID No. 66; coding for enzyme E_{d} and an enzyme Eₑ), fadD (SEQ ID No. 21; coding for enzyme Eₐ), fadE (SEQ ID No. 40; coding for enzyme E_{b}) and fadL (SEQ ID No. 68; coding for an enzyme which catalyses the transport of fatty acid methyl esters across the outer membrane). The genes fadA, fadB, fadD, fadE and fadL are replaced by a kanamycin cassette. Before the provision of the strain with the expression vectors by means of a helper plasmid, which codes for the Flp recombinase, this is removed in a manner known to those skilled in the art (see Datsenko K. A. and Wanner B. L. (2000) PNAS 97(12):6640-6645), resulting in strains JW5578-1 Kan^{S}, JW3822-1 Kan^{S}, JW1794-1 Kan^{S}, JW5020-1 Kan^{S} and JW2341-1 Kan^{S}.

BW25113, JW5578-1 Kan^{S}, JW3822-1 Kan^{S}, JW1794-1 Kan^{S}, JW5020-1 Kan^{S} and JW2341-1 Kan^{S} are sequentially transformed with the plasmids
1. pBT10 (construction described in practical example B.2 of PCT/EP2008/067447), and
2. pJ294[alaDH_B.s._TA_C.v.(Ct)_Ptac-synUcTE] (SEQ ID No. 38), pJ294[alaDH_B.s._TA_C.v.(Ct)_Ptac-ChFATB2] (SEQ ID No. 70), pJ294[alaDH_B.s._TA_C.v.(Ct)_Ptac-CnFATB3] (SEQ ID No. 71), or pJ294[alaDH_B.s._TA_C.v.(Ct)_Ptac-CPF_{—}2954] (SEQ ID No. 72), and
3. pCDF[wax-dgaT_AsADP1(co_Ec)-fadD_Ec] (SEQ ID No. 16) or pCDF[atfA1_Ab(co_Ec)-fadD_Ec] (SEQ ID No. 17)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmids pJ294[alaDH_B.s._TA_C.v.(Ct)_Ptac-ChFATB2] (SEQ ID No. 38), pJ294[alaDH_B.s._TA_C.v.(Ct)_Ptac-CnFATB3] (SEQ ID No. 70) and pJ294[alaDH_B.s._TA_C.v.(Ct)_Ptac-CPF_{—}2954] (SEQ ID No. 71) are created starting from the plasmid pJ294[alaDH_B.s._TA_C.v.(Ct)_Ptac-synUcTE] (SEQ ID No. 72), in that the gene coding for the thioesterase synUcTE together with P_{tac} and a 3′ flanking region is cut out from the vector via BamHI/NotI and replaced by the genes coding for the thioesterases ChFATB2 (SEQ ID No. 59) CnFATB3 (SEQ ID No. 61) or CPF_{—}2954 (SEQ ID No. 63) (incl. P_{tac} and identical 3′ flanking region). These fragments are created by gene synthesis, wherein the regions coding for ChFATB2 and CnFATB3 are codon-optimized for translation in E. coli, but the region coding for CPF_{—}2954 is not codon-optimized, but instead the wild type sequence is used.

In this manner, inter alia the following strains are constructed:
E. coli BW25113 pBT10/pJ294[alaDH_B.s._TA_C.v.(Ct)_Ptac-synUcTE]/pCDF[wax-dgaT_AsADP1(co_Ec)-fadD_Ec]
E. coli BW25113 pBT10/pJ294[alaDH_B.s._TA_C.v.(Ct)_Ptac-synUcTE]/pCDF[atfA1_Ab(co_Ec)-fadD_Ec]
E. coli JW5578-1 Kan^{S} pBT10/pJ294[alaDH_B.s._TA_C.v.(Ct)_Ptac-synUcTE]/pCDF[wax-dgaT_AsADP1(co_Ec)-fadD_Ec]
E. coli JW5578-1 Kan^{S} pBT10/pJ294[alaDH_B.s._TA_C.v.(Ct)_Ptac-synUcTE]/pCDF[atfA1_Ab(co_Ec)-fadD_Ec]
E. coli JW3822-1 Kan^{S} pBT10/pJ294[alaDH_B.s._TA_C.v.(Ct)_Ptac-synUcTE]/pCDF[wax-dgaT_AsADP1(co_Ec)-fadD_Ec]
E. coli JW3822-1 Kan^{S} pBT10/pJ294[alaDH_B.s._TA_C.v.(Ct)_Ptac-synUcTE]/pCDF[atfA1_Ab(co_Ec)-fadD_Ec]
E. coli JW1794-1 Kan^{S} pBT10/pJ294[alaDH_B.s._TA_C.v.(Ct)_Ptac-synUcTE]/pCDF[wax-dgaT_AsADP1(co_Ec)-fadD_Ec]
E. coli JW1794-1 Kan^{S} pBT10/pJ294[alaDH_B.s._TA_C.v.(Ct)_Ptac-synUcTE]/pCDF[atfA1_Ab(co_Ec)-fadD_Ec]
E. coli JW5020-1 Kan^{S} pBT10/pJ294[alaDH_B.s._TA_C.v.(Ct)_Ptac-synUcTE]/pCDF[wax-dgaT_AsADP1(co_Ec)-fadD_Ec]
E. coli JW5020-1 Kan^{S} pBT10/pJ294[alaDH_B.s._TA_C.v.(Ct)_Ptac-synUcTE]/pCDF[atfA1_Ab(co_Ec)-fadD_Ec]
E. coli JW2341-1 Kan^{S} pBT10/pJ294[alaDH_B.s._TA_C.v.(Ct)_Ptac-synUcTE]/pCDF[wax-dgaT_AsADP1(co_Ec)-fadD_Ec]
E. coli JW2341-1 Kan^{S} pBT10/pJ294[alaDH_B.s._TA_C.v.(Ct)_Ptac-synUcTE]/pCDF[atfA1_Ab(co_Ec)-fadD_Ec]

These strains are subjected to a fed-batch fermentation in order to analyse their capacity for production of methyl hydroxylaurate, methyl oxolaurate, methyl carboxylaurate and methyl aminolaurate and methyl hydroxytetradecanoate, methyl oxotetradecanoate, methyl carboxytetradecanoate and methyl aminotetradecanoate from glucose. This is performed with an 8-fold parallel fermentation system from DASGIP, as described in Example 8.

It is shown that these strains, with addition of 1% (v/v) methanol, are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate from glucose.

Further, the following strains are constructed:
E. coli BW25113 pBT10/pJ294[alaDH_B.s._TA_C.v.(Ct)_Ptac-ChFATB2]/pCDF[wax-dgaT_AsADP1(co_Ec)-fadD_Ec]
E. coli BW25113 pBT10/pJ294[alaDH_B.s._TA_C.v.(Ct)_Ptac-ChFATB2]/pCDF[atfA1_Ab(co_Ec)-fadD_Ec]
E. coli JW5578-1 Kan^{S} pBT10/pJ294[alaDH_B.s._TA_C.v.(Ct)_Ptac-ChFATB2]/pCDF[wax-dgaT_AsADP1(co_Ec)-fadD_Ec]
E. coli JW5578-1 Kan^{S} pBT10/pJ294[alaDH_B.s._TA_C.v.(Ct)_Ptac-ChFATB2]/pCDF[atfA1_Ab(co_Ec)-fadD_Ec]
E. coli JW3822-1 Kan^{S} pBT10/pJ294[alaDH_B.s._TA_C.v.(Ct)_Ptac-ChFATB2]/pCDF[wax-dgaT_AsADP1(co_Ec)-fadD_Ec]
E. coli JW3822-1 Kan^{S} pBT10/pJ294[alaDH_B.s._TA_C.v.(Ct)_Ptac-ChFATB2]/pCDF[atfA1_Ab(co_Ec)-fadD_Ec]
E. coli JW1794-1 Kan^{S} pBT10/pJ294[alaDH_B.s._TA_C.v.(Ct)_Ptac-ChFATB2]/pCDF[wax-dgaT_AsADP1(co_Ec)-fadD_Ec]
E. coli JW1794-1 Kan^{S} pBT10/pJ294[alaDH_B.s._TA_C.v.(Ct)_Ptac-ChFATB2]/pCDF[atfA1_Ab(co_Ec)-fadD_Ec]
E. coli JW5020-1 Kan^{S} pBT10/pJ294[alaDH_B.s._TA_C.v.(Ct)_Ptac-ChFATB2]/pCDF[wax-dgaT_AsADP1(co_Ec)-fadD_Ec]
E. coli JW5020-1 Kan^{S} pBT10/pJ294[alaDH_B.s._TA_C.v.(Ct)_Ptac-ChFATB2]/pCDF[atfA1_Ab(co_Ec)-fadD_Ec]
E. coli JW2341-1 Kan^{S} pBT10/pJ294[alaDH_B.s._TA_C.v.(Ct)_Ptac-ChFATB2]/pCDF[wax-dgaT_AsADP1(co_Ec)-fadD_Ec]
E. coli JW2341-1 Kan^{S} pBT10/pJ294[alaDH_B.s._TA_C.v.(Ct)_Ptac-ChFATB2]/pCDF[atfA1_Ab(co_Ec)-fadD_Ec]

These strains are subjected to a fed-batch fermentation in order to analyse their capacity for production of methyl hydroxyoctanoate, methyl oxooctanoate, methyl carboxyoctanoate and methyl aminooctanoate and methyl hydroxydecanoate, methyl oxodecanoate, methyl carboxydecanoate and methyl aminodecanoate from glucose. This is performed with an 8-fold parallel fermentation system from DASGIP, as described in Example 8.

It is shown that these strains, with addition of 1% (v/v) methanol, are capable of forming methyl octanoate, methyl hydroxyoctanoate, methyl oxooctanoate, methyl carboxyoctanoate and methyl aminooctanoate and methyl decanoate, methyl hydroxydecanoate, methyl oxodecanoate, methyl carboxydecanoate and methyl aminodecanoate from glucose.

Further, the following strains are constructed:
E. coli BW25113 pBT10/pJ294[alaDH_B.s._TA_C.v.(Ct)_Ptac-CnFATB3]/pCDF[wax-dgaT_AsADP1(co_Ec)-fadD_Ec]
E. coli BW25113 pBT10/pJ294[alaDH_B.s._TA_C.v.(Ct)_Ptac-CnFATB3]/pCDF[atfA1_Ab(co_Ec)-fadD_Ec]
E. coli JW5578-1 Kan^{S} pBT10/pJ294[alaDH_B.s._TA_C.v.(Ct)_Ptac-CnFATB3]/pCDF[wax-dgaT_AsADP1(co_Ec)-fadD_Ec]
E. coli JW5578-1 Kan^{S} pBT10/pJ294[alaDH_B.s._TA_C.v.(Ct)_Ptac-CnFATB3]/pCDF[atfA1_Ab(co_Ec)-fadD_Ec]
E. coli JW3822-1 Kan^{S} pBT10/pJ294[alaDH_B.s._TA_C.v.(Ct)_Ptac-CnFATB3]/pCDF[wax-dgaT_AsADP1(co_Ec)-fadD_Ec]
E. coli JW3822-1 Kan^{S} pBT10/pJ294[alaDH_B.s._TA_C.v.(Ct)_Ptac-CnFATB3]/pCDF[atfA1_Ab(co_Ec)-fadD_Ec]
E. coli JW1794-1 Kan^{S} pBT10/pJ294[alaDH_B.s._TA_C.v.(Ct)_Ptac-CnFATB3]/pCDF[wax-dgaT_AsADP1(co_Ec)-fadD_Ec]
E. coli JW1794-1 Kan^{S} pBT10/pJ294[alaDH_B.s._TA_C.v.(Ct)_Ptac-CnFATB3]/pCDF[atfA1_Ab(co_Ec)-fadD_Ec]
E. coli JW5020-1 Kan^{S} pBT10/pJ294[alaDH_B.s._TA_C.v.(Ct)_Ptac-CnFATB3]/pCDF[wax-dgaT_AsADP1(co_Ec)-fadD_Ec]
E. coli JW5020-1 Kan^{S} pBT10/pJ294[alaDH_B.s._TA_C.v.(Ct)_Ptac-CnFATB3]/pCDF[atfA1_Ab(co_Ec)-fadD_Ec]
E. coli JW2341-1 Kan^{S} pBT10/pJ294[alaDH_B.s._TA_C.v.(Ct)_Ptac-CnFATB3]/pCDF[wax-dgaT_AsADP1(co_Ec)-fadD_Ec]
E. coli JW2341-1 Kan^{S} pBT10/pJ294[alaDH_B.s._TA_C.v.(Ct)_Ptac-CnFATB3]/pCDF[atfA1_Ab(co_Ec)-fadD_Ec]

These strains are subjected to a fed-batch fermentation in order to analyse their capacity for production of methyl hydroxylaurate, methyl oxolaurate, methyl carboxylaurate and methyl aminolaurate and methyl hydroxytetradecanoate, methyl oxotetradecanoate, methyl carboxytetradecanoate and methyl aminotetradecanoate and methyl hydroxy-9-hexadecenoate, methyl oxo-9-hexadecenoate, methyl carboxy-9-hexadecenoate and methyl amino-9-hexadecenoate from glucose. This is performed with an 8-fold parallel fermentation system from DASGIP, as described in Example 8.

It is shown that these strains, with addition of 1% (v/v) methanol, are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate and methyl 9-hexadecenoate, methyl hydroxy-9-hexadecenoate, methyl oxo-9-hexadecenoate, methyl carboxy-9-hexadecenoate and methyl amino-9-hexadecenoate from glucose.

Finally, the following strains are constructed:
E. coli BW25113 pBT10/pJ294[alaDH_B.s._TA_C.v.(Ct)_Ptac-CPF_{—}2954]/pCDF[wax-dgaT_AsADP1(co_Ec)-fadD_Ec]
E. coli BW25113 pBT10/pJ294[alaDH_B.s._TA_C.v.(Ct)_Ptac-CPF_{—}2954]/pCDF[atfA1_Ab(co_Ec)-fadD_Ec]
E. coli JW5578-1 Kan^{S} pBT10/pJ294[alaDH_B.s._TA_C.v.(Ct)_Ptac-CPF_{—}2954]/pCDF[wax-dgaT_AsADP1(co_Ec)-fadD_Ec]
E. coli JW5578-1 Kan^{S} pBT10/pJ294[alaDH_B.s._TA_C.v.(Ct)_Ptac-CPF_{—}2954]/pCDF[atfA1_Ab(co_Ec)-fadD_Ec]
E. coli JW3822-1 Kan^{S} pBT10/pJ294[alaDH_B.s._TA_C.v.(Ct)_Ptac-CPF_{—}2954]/pCDF[wax-dgaT_AsADP1(co_Ec)-fadD_Ec]
E. coli JW3822-1 Kan^{S} pBT10/pJ294[alaDH_B.s._TA_C.v.(Ct)_Ptac-CPF_{—}2954]/pCDF[atfA1_Ab(co_Ec)-fadD_Ec]
E. coli JW1794-1 Kan^{S} pBT10/pJ294[alaDH_B.s._TA_C.v.(Ct)_Ptac-CPF_{—}2954]/pCDF[wax-dgaT_AsADP1(co_Ec)-fadD_Ec]
E. coli JW1794-1 Kan^{S} pBT10/pJ294[alaDH_B.s._TA_C.v.(Ct)_Ptac-CPF_{—}2954]/pCDF[atfA1_Ab(co_Ec)-fadD_Ec]
E. coli JW5020-1 Kan^{S} pBT10/pJ294[alaDH_B.s._TA_C.v.(Ct)_Ptac-CPF_{—}2954]/pCDF[wax-dgaT_AsADP1(co_Ec)-fadD_Ec]
E. coli JW5020-1 Kan^{S} pBT10/pJ294[alaDH_B.s._TA_C.v.(Ct)_Ptac-CPF_{—}2954]/pCDF[atfA1_Ab(co_Ec)-fadD_Ec]
E. coli JW2341-1 Kan^{S} pBT10/pJ294[alaDH_B.s._TA_C.v.(Ct)_Ptac-CPF_{—}2954]/pCDF[wax-dgaT_AsADP1(co_Ec)-fadD_Ec]
E. coli JW2341-1 Kan^{S} pBT10/pJ294[alaDH_B.s._TA_C.v.(Ct)_Ptac-CPF_{—}2954]/pCDF[atfA1_Ab(co_Ec)-fadD_Ec]

These strains are subjected to a fed-batch fermentation in order to analyse their capacity for production of methyl hydroxyoctanoate, methyl oxooctanoate, methyl carboxyoctanoate and methyl aminooctanoate and methyl hydroxyhexanoate, methyl oxohexanoate, methyl carboxyhexanoate and methyl aminohexanoate from glucose. This is performed with an 8-fold parallel fermentation system from DASGIP, as described in Example 8.

It is shown that these strains, with addition of 1% (v/v) methanol, are capable of forming methyl octanoate, methyl hydroxyoctanoate, methyl oxooctanoate, methyl carboxyoctanoate and methyl aminooctanoate and methyl hexanoate, methyl hydroxyhexanoate, methyl oxohexanoate, methyl carboxyhexanoate and methyl aminohexanoate from glucose.

### Example 11

### Production of Aminohexanoic Acid, Aminooctanoic Acid, Aminodecanoic Acid, Aminotetradecanoic Acid and Amino-9-Hexadecenoic Acid by E. coli Strains with Deletion of One of the Genes fadA, fadB, fadD, fadE or fadL and Expression Vectors for the Genes synUcTE from Umbellularia californica, ChFATB2 from Cuphea hookeriana, CnFATB3 from Cocos nucifera or CPF 2954 from Clostridium perfringens (Genbank Acc. # ABG82470 SEQ-ID XY) and Ald from Bacillus subtilis, Cv_{—}2025 from Chromobacterium violaceum in Combination with an Expression Vector for the Genes alkB, alkG and alkT from the Alk Operon of Pseudomonas putida

To create E. coli strains with expression vectors for the genes synUcTE from Umbellularia californica, ChFATB2 from Cuphea hookeriana (SEQ ID No. 58), CnFATB3 from Cocos nucifera (SEQ ID No. 60) or CPF_{—}2954 from Clostridium perfringens (SEQ ID No. 62) and ald from Bacillus subtilis, and Cv_{—}2025 from Chromobacterium violaceum in combination with an expression vector for the genes alkB, alkG and alkT from the alk operon of Pseudomonas putida GPo1, electrocompetent cells of E. coli JW5578-1 Kan^{S}, JW3822-1 Kan^{S}, JW1794-1 Kan^{S}, JW5020-1 Kan^{S} and JW2341-1 Kan^{S} are produced. This takes place in a manner known to those skilled in the art.

JW5578-1 Kan^{S}, JW3822-1 Kan^{S}, JW1794-1 Kan^{S}, JW5020-1 Kan^{S} and JW2341-1 Kan^{S} are sequentially transformed with the plasmids
1. pBT10 (construction described in practical example B.2 of PCT/EP2008/067447), and
2. pJ294[alaDH_B.s._TA_C.v.(Ct)_Ptac-synUcTE] (SEQ ID No. 38), pJ294[alaDH_B.s._TA_C.v.(Ct)_Ptac-ChFATB2] (SEQ ID No. 70), pJ294[alaDH_B.s._TA_C.v.(Ct)_Ptac-CnFATB3] (SEQ ID No. 71), or pJ294[alaDH_B.s._TA_C.v.(Ct)_Ptac-CPF_{—}2954] (SEQ ID No. 72)
   and plated out onto LB agar plates containing kanamycin (50 μg/ml) and ampicillin (100 μg/ml). Transformants are checked for the presence of the correct plasmids by plasmid preparation and analytical restriction analysis.

In this manner, inter alia the following strains are constructed:
E. coli BW25113 pBT10/pJ294[alaDH_B.s._TA_C.v.(Ct)_Ptac-synUcTE]
E. coli JW5578-1 Kan^{S} pBT10/pJ294[alaDH_B.s._TA_C.v.(Ct)_Ptac-synUcTE]
E. coli JW3822-1 Kan^{S} pBT10/pJ294[alaDH_B.s._TA_C.v.(Ct)_Ptac-synUcTE]
E. coli JW1794-1 Kan^{S} pBT10/pJ294[alaDH_B.s._TA_C.v.(Ct)_Ptac-synUcTE]
E. coli JW5020-1 Kan^{S} pBT10/pJ294[alaDH_B.s._TA_C.v.(Ct)_Ptac-synUcTE]
E. coli JW2341-1 Kan^{S} pBT10/pJ294[alaDH_B.s._TA_C.v.(Ct)_Ptac-synUcTE]

These strains are subjected to a fed-batch fermentation in order to analyse their capacity for production of hydroxylauric acid, oxolauric acid, carboxylauric acid and aminolauric acid and hydroxytetradecanoic acid, oxotetradecanoic acid, carboxytetradecanoic acid and aminotetradecanoic acid from glucose. This is performed with an 8-fold parallel fermentation system from DASGIP, as described in Example 8.

It is shown that these strains are capable of forming lauric acid, hydroxylauric acid, oxolauric acid, carboxylauric acid and aminolauric acid and tetradecanoic acid, hydroxytetradecanoic acid, oxotetradecanoic acid, carboxytetradecanoic acid and aminotetradecanoic acid from glucose.

Further, the following strains are constructed:
E. coli BW25113 pBT10/pJ294[alaDH_B.s._TA_C.v.(Ct)_Ptac-ChFATB2]
E. coli JW5578-1 Kan^{S} pBT10/pJ294[alaDH_B.s._TA_C.v.(Ct)_Ptac-ChFATB2]
E. coli JW3822-1 Kan^{S} pBT10/pJ294[alaDH_B.s._TA_C.v.(Ct)_Ptac-ChFATB2]
E. coli JW1794-1 Kan^{S} pBT10/pJ294[alaDH_B.s._TA_C.v.(Ct)_Ptac-ChFATB2]
E. coli JW5020-1 Kan^{S} pBT10/pJ294[alaDH_B.s._TA_C.v.(Ct)_Ptac-ChFATB2]/
E. coli JW2341-1 Kan^{S} pBT10/pJ294[alaDH_B.s._TA_C.v.(Ct)_Ptac-ChFATB2]

These strains are subjected to a fed-batch fermentation in order to analyse their capacity for production of hydroxyoctanoic acid, oxooctanoic acid, carboxyoctanoic acid and aminooctanoic acid and hydroxydecanoic acid, oxodecanoic acid, carboxydecanoic acid and aminodecanoic acid from glucose. This is performed with an 8-fold parallel fermentation system from DASGIP, as described in Example 8.

It is shown that these strains are capable of forming octanoic acid, hydroxyoctanoic acid, oxooctanoic acid, carboxyoctanoic acid and aminooctanoic acid and decanoic acid, hydroxydecanoic acid, oxodecanoic acid, carboxydecanoic acid and aminodecanoic acid from glucose.

Further, the following strains are constructed:
E. coli BW25113 pBT10/pJ294[alaDH_B.s._TA_C.v.(Ct)_Ptac-CnFATB3]
E. coli JW5578-1 Kan^{S} pBT10/pJ294[alaDH_B.s._TA_C.v.(Ct)_Ptac-CnFATB3]
E. coli JW3822-1 Kan^{S} pBT10/pJ294[alaDH_B.s._TA_C.v.(Ct)_Ptac-CnFATB3]
E. coli JW1794-1 Kan^{S} pBT10/pJ294[alaDH_B.s._TA_C.v.(Ct)_Ptac-CnFATB3]
E. coli JW5020-1 Kan^{S} pBT10/pJ294[alaDH_B.s._TA_C.v.(Ct)_Ptac-CnFATB3]
E. coli JW2341-1 Kan^{S} pBT10/pJ294[alaDH_B.s._TA_C.v.(Ct)_Ptac-CnFATB3]

These strains are subjected to a fed-batch fermentation in order to analyse their capacity for production of hydroxylauric acid, oxolauric acid, carboxylauric acid and aminolauric acid and hydroxytetradecanoic acid, oxotetradecanoic acid, carboxytetradecanoic acid and aminotetradecanoic acid and hydroxy-9-hexadecenoic acid, oxo-9-hexadecenoic acid, carboxy-9-hexadecenoic acid and amino-9-hexadecenoic acid from glucose. This is performed with an 8-fold parallel fermentation system from DASGIP, as described in Example 8.

It is shown that these strains are capable of forming lauric acid, hydroxylauric acid, oxolauric acid, carboxylauric acid and aminolauric acid and tetradecanoic acid, hydroxytetradecanoic acid, oxotetradecanoic acid, carboxytetradecanoic acid and aminotetradecanoic acid and 9-hexadecenoic acid, hydroxy-9-hexadecenoic acid, oxo-9-hexadecenoic acid, carboxy-9-hexadecenoic acid and amino-9-hexadecenoic acid from glucose.

Finally, the following strains are constructed:
E. coli BW25113 pBT10/pJ294[alaDH_B.s._TA_C.v.(Ct)_Ptac-CPF_{—}2954]
E. coli JW5578-1 Kan^{S} pBT10/pJ294[alaDH_B.s._TA_C.v.(Ct)_Ptac-CPF_{—}2954]
E. coli JW3822-1 Kan^{S} pBT10/pJ294[alaDH_B.s._TA_C.v.(Ct)_Ptac-CPF_{—}2954]
E. coli JW1794-1 Kan^{S} pBT10/pJ294[alaDH_B.s._TA_C.v.(Ct)_Ptac-CPF_{—}2954]
E. coli JW5020-1 Kan^{S} pBT10/pJ294[alaDH_B.s._TA_C.v.(Ct)_Ptac-CPF_{—}2954]
E. coli JW2341-1 Kan^{S} pBT10/pJ294[alaDH_B.s._TA_C.v.(Ct)_Ptac-CPF_{—}2954]

These strains are subjected to a fed-batch fermentation in order to analyse their capacity for production of hydroxyoctanoic acid, oxooctanoic acid, carboxyoctanoic acid and aminooctanoic acid and hydroxyhexanoic acid, oxohexanoic acid, carboxyhexanoic acid and aminohexanoic acid from glucose. This is performed with an 8-fold parallel fermentation system from DASGIP, as described in Example 8.

It is shown that these strains are capable of forming octanoic acid, hydroxyoctanoic acid, oxooctanoic acid, carboxyoctanoic acid and aminooctanoic acid and hexanoic acid, hydroxyhexanoic acid, oxohexanoic acid, carboxyhexanoic acid and aminohexanoic acid from glucose.

### Example 12

### Production of Methyl Aminolaurate by an E. coli Strain with Deletion in the Gene fadE and Expression Vectors for the Genes synUcTE from Umbellularia Californica, Ald from Bacillus subtilis, Psyr_{—}4866 from Pseudomonas syringae pv. syringae B728a, PFL_{—}5927 from Pseudomonas protegens Pf-5, PSPPH 4896 from Pseudomonas syringae pv. phaseolicola 1448A or PSPTOT1_{—}2473 from Pseudomonas syringae pv. tomato T1 in Combination with an Expression Vector for the Genes alkB, alkG, alkT and alkL from the Alk Operon of Pseudomonas putida and an Expression Vector for the Genes fadD from Escherichia coli and Wax-dgaT from Acinetobacter sp. ADP1 or atfA1 from Alcanivorax borkumensis

To create E. coli strains with expression vectors for the genes synUcTE from Umbellularia californica, ald from Bacillus subtilis, Psyr_{—}4866 from Pseudomonas syringae pv. syringae B728a (SEQ ID No. 73), PFL_{—}5927 from Pseudomonas protegens Pf-5 (SEQ ID No. 75), PSPPH_{—}4896 from Pseudomonas syringae pv. phaseolicola 1448A (SEQ ID No. 77) or PSPTOT1_{—}2473 from Pseudomonas syringae pv. tomato T1 (SEQ ID No. 79) in combination with an expression vector for the genes alkB, alkG and alkT from the alk operon of Pseudomonas putida GPo1 and an expression vector for the genes fadD from Escherichia coli and wax-dgaT from Acinetobacter sp. ADP1 or atfA1 from Alcanivorax borkumensis, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[alaDH_B.s._TA_Psyr_{—}4866_Ptac-synUcTE] (SEQ ID No. 81), pJ294[alaDH_B.s._TA_PFL_{—}5927_Ptac-synUcTE] (SEQ ID No. 82), pJ294[alaDH_B.s._TA_PSPPH_{—}4896_Ptac-synUcTE] (SEQ ID No. 83), or pJ294[alaDH_B.s._TA_PSPTOT1_{—}2473_Ptac-synUcTE] (SEQ ID No. 84), and
3. pCDF[wax-dgaT_AsADP1(co_Ec)-fadD_Ec] (SEQ ID No. 16) or pCDF[affA1_Ab(co_Ec)-fadD_Ec] (SEQ ID No. 17)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmids pJ294[alaDH_B.s._Psyr_{—}4866_Ptac-synUcTE] (SEQ ID No. 81), pJ294[alaDH_B.s._TA_PFL_{—}5927_Ptac-synUcTE] (SEQ ID No. 82), pJ294[alaDH_B.s._TA_PSPPH_{—}4896_Ptac-synUcTE] (SEQ ID No. 83) and pJ294[alaDH_B.s._TA_PSPTOT1_{—}2473_Ptac-synUcTE] (SEQ ID No. 84) are created starting from the plasmid pJ294[alaDH_B.s._TA_C.v.(Ct)_Ptac-synUcTE] (SEQ ID No. 38), in that the gene coding for the transaminase Cv_{—}2025 with the 3′ end of the ald gene from Bacillus subtilis and a 3′ flanking region is cut out from the vector via BglII FseI and replaced by the genes coding for the transaminases Psyr_{—}4866 (SEQ ID No. 74), PFL_{—}5927 (SEQ ID No. 76), PSPPH_{—}4896 (SEQ ID No. 78) or PSPTOT1_{—}2473 (SEQ ID No. 80) (incl. the 3′ end of the ald gene from Bacillus subtilis and identical 3′ flanking region). These fragments are created by gene synthesis, wherein the regions coding for Psyr_{—}4866, PFL_{—}5927, PSPPH_{—}4896 and PSPTOT1_{—}2473 are codon-optimized for translation in E. coli.

In this manner, the following strains are constructed:
E. coli JW5020-1 Kan^{S} pBT10/pJ294[alaDH_B.s._Psyr_{—}4866_Ptac-synUcTE]/pCDF[wax-dgaT_AsADP1(co_Ec)-fadD_Ec]
E. coli JW5020-1 Kan^{S} pBT10/pJ294[alaDH_B.s._Psyr_{—}4866_Ptac-synUcTE]/pCDF[atfA1_Ab(co_Ec)-fadD_Ec]
E. coli JW5020-1 Kan^{S} pBT10/pJ294[alaDH_B.s._TA_PFL_{—}5927_Ptac-synUcTE]/pCDF[wax-dgaT_AsADP1(co_Ec)-fadD_Ec]
E. coli JW5020-1 Kan^{S} pBT10/pJ294[alaDH_B.s._TA_PFL_{—}5927_Ptac-synUcTE]/pCDF[atfA1_Ab(co_Ec)-fadD_Ec]
E. coli JW5020-1 Kan^{S} pBT10/pJ294[alaDH_B.s._TA_PSPPH_{—}4896_Ptac-synUcTE]/pCDF[wax-dgaT_AsADP1(co_Ec)-fadD_Ec]
E. coli JW5020-1 Kan^{S} pBT10/pJ294[alaDH_B.s._TA_PSPPH_{—}4896_Ptac-synUcTE]/pCDF[atfA1_Ab(co_Ec)-fadD_Ec]
E. coli JW5020-1 Kan^{S} pBT10/pJ294[alaDH_B.s._TA_PSPTOT1_{—}2473_Ptac-synUcTE]/pCDF[wax-dgaT_AsADP1(co_Ec)-fadD_Ec]
E. coli JW5020-1 Kan^{S} pBT10/pJ294[alaDH_B.s._TA_PSPTOT1_{—}2473_Ptac-synUcTE]/pCDF[atfA1_Ab(co_Ec)-fadD_Ec]

These strains are subjected to a fed-batch fermentation in order to analyse their capacity for production of methyl hydroxylaurate, methyl oxolaurate, methyl carboxylaurate and methyl aminolaurate and methyl hydroxytetradecanoate, methyl oxotetradecanoate, methyl carboxytetradecanoate and methyl aminotetradecanoate from glucose. This is performed with an 8-fold parallel fermentation system from DASGIP, as described in Example 8.

It is shown that these strains, with addition of 1% (v/v) methanol, are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate from glucose.

### Example 13

### Production of aminolauric acid by an E. coli strain with deletion in the gene fadE and expression vectors for the genes synUcTE from Umbellularia californica, ald from Bacillus subtilis, Psyr_{—}4866 from Pseudomonas syringae pv. syringae 8728a, PFL_{—}5927 from Pseudomonas protegens Pf-5, PSPPH 4896 from Pseudomonas syringae pv. phaseolicola 1448A or PSPTOT1_{—}2473 from Pseudomonas syringae pv. tomato T1 in Combination with an Expression Vector for the Genes alkB, alkG, alkT and alkL from the Alk Operon of Pseudomonas putida

To create E. coli strains with expression vectors for the genes synUcTE from Umbellularia californica, ald from Bacillus subtilis, Psyr_{—}4866 from Pseudomonas syringae pv. syringae B728a (SEQ ID No. 73), PFL_{—}5927 from Pseudomonas protegens Pf-5 (SEQ ID No. 75), PSPPH_{—}4896 from Pseudomonas syringae pv. phaseolicola 1448A (SEQ ID No. 77) or PSPTOT1_{—}2473 from Pseudomonas syringae pv. tomato T1 (SEQ ID No. 79) in combination with an expression vector for the genes alkB, alkG and alkT from the alk operon of Pseudomonas putida GPo1, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[alaDH_B.s._TA_Psyr_{—}4866_Ptac-synUcTE] (SEQ ID No. 81), pJ294[alaDH_B.s._TA_PFL_{—}5927_Ptac-synUcTE] (SEQ ID No. 82), pJ294[alaDH_B.s._TA_PSPPH_{—}4896_Ptac-synUcTE] (SEQ ID No. 83), or pJ294[alaDH_B.s._TA_PSPTOT1_{—}2473_Ptac-synUcTE] (SEQ ID No. 84)
   and plated out onto LB agar plates containing kanamycin (50 μg/ml) and ampicillin (100 μg/ml) t. Transformants are checked for the presence of the correct plasmids by plasmid preparation and analytical restriction analysis.

In this manner, the following strains are constructed:
E. coli JW5020-1 Kan^{S} pBT10/pJ294[alaDH_B.s._Psyr_{—}4866_Ptac-synUcTE]
E. coli JW5020-1 Kan^{S} pBT10/pJ294[alaDH_B.s._TA_PFL_{—}5927_Ptac-synUcTE]
E. coli JW5020-1 Kan^{S} pBT10pJ294[alaDH_B.s._TA_PSPPH_{—}4896_Ptac-synUcTE]
E. coli JW5020-1 Kan^{S} pBT10pJ294[alaDH_B.s._TA_PSPTOT1_{—}2473_Ptac-synUcTE]

These strains are subjected to fed-batch fermentation in order to analyse their capacity for production of hydroxylauric acid, oxolauric acid, carboxylauric acid and aminolauric acid and hydroxytetradecanoic acid, oxotetradecanoic acid, carboxytetradecanoic acid and aminotetradecanoic acid from glucose. This is performed with an 8-fold parallel fermentation system from DASGIP, as described in Example 8.

It is shown that these strains are capable of forming lauric acid, hydroxylauric acid, oxolauric acid, carboxylauric acid and aminolauric acid and tetradecanoic acid, hydroxytetradecanoic acid, oxotetradecanoic acid, carboxytetradecanoic acid and aminotetradecanoic acid from glucose.

### Example 14

### Production of Methyl Aminolaurate by an E. coli Strain with Deletion in the Gene fadE and Expression Vectors for the Genes synUcTE from Umbellularia californica, alr2355 from Nostoc sp. PCC 7120, Rleg_{—}1610 from Rhizobium leguminasorum bv. trifolii WSM1325, blr1738 (aldA) from Bradyrhizobium japonicum USDA 110 or BMD 5199 from Bacillus megaterium DSM 319 and Cv_{—}2025 from Chromobacterium violaceum in Combination with an Expression Vector for the Genes alkB, alkG, alkT and alkL from the alk Operon of Pseudomonas putida and an Expression Vector for the Genes fadD from Escherichia coli and Wax-dgaT from Acinetobacter sp. ADP1 or atfA1 from Alcanivorax borkumensis

To create E. coli strains with expression vectors for the genes synUcTE from Umbellularia californica, alr2355 from Nostoc sp. PCC 7120 (SEQ ID No. 85), Rleg 1610 from Rhizobium leguminasorum bv. trifolii WSM1325 (SEQ ID No. 87), blr1738 (aldA) from Bradyrhizobium japonicum USDA 110 (SEQ ID No. 89) or BMD 5199 from Bacillus megaterium DSM 319 (SEQ ID No. 91) and Cv_{—}2025 from Chromobacterium violaceum in combination with an expression vector for the genes alkB, alkG and alkT from the alk operon of Pseudomonas putida GPo1 and an expression vector for the genes fadD from Escherichia coli and wax-dgaT from Acinetobacter sp. ADP1 or atfA1 from Alcanivorax borkumensis, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[alr2355_TA_C.v.(Ct)_Ptac-synUcTE] (SEQ ID No. 93), pJ294[Rleg_{—}1610_TA_C.v.(Ct)_Ptac-synUcTE] (SEQ ID No. 94), pJ294[blr1738TA_C.v.(Ct)_Ptac-synUcTE] (SEQ ID No. 95), or pJ294[BMD_{—}5199_TA_C.v.(Ct)_Ptac-synUcTE] (SEQ ID No. 96),
   and
3. pCDF[wax-dgaT_AsADP1(co_Ec)-fadD_Ec] (SEQ ID No. 16) bzw. pCDF[atfA1_Ab(co_Ec)-fadD_Ec] (SEQ ID No. 17)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmids pJ294[alr2355_TA_C.v.(Ct)_Ptac-synUcTE] (SEQ ID No. 93), pJ294[Rleg_{—}1610_TA_C.v.(Ct)_Ptac-synUcTE] (SEQ ID No. 94), pJ294[blr1738_TA_C.v.(Ct)_Ptac-synUcTE] (SEQ ID No. 95) and pJ294[BMD_{—}5199_TA_C.v.(Ct)_Ptac-synUcTE] (SEQ ID No. 96) are created starting from the plasmid pJ294[alaDH_B.s._TA_C.v.(Ct)_Ptac-synUcTE] (SEQ ID No. 38, in that the gene coding for the alanine dehydrogenase from Bacillus subtilis together with the P_{lacUV5} and the 5′ end of the Cv_{—}2025 gene from Chromobacterium violaceum is cut out of the vector via PstI/EcoNI and replaced by the genes coding for the alanine dehydrogenases alr2355 (SEQ ID No. 86), Rleg_{—}1610 (SEQ ID No. 88), blr1738 (SEQ ID No. 90) or BMD_{—}5199 (SEQ ID No. 92) (incl. P_{lacUV5} and the 5′ end of the Cv_{—}2025 gene from Chromobacterium violaceum). These fragments are created by gene synthesis, wherein the regions coding for alr2355, Rleg_{—}1610, blr1738 and BMD_{—}5199 are not codon-optimized for translation in E. coli, but instead the wild type sequences are used.

In this manner, the following strains are constructed:
E. coli JW5020-1 Kan^{S} pBT10/pJ294[alr2355_TA_C.v.(Ct)_Ptac-synUcTE]/pCDF[wax-dgaT_AsADP1(co_Ec)-fadD_Ec]
E. coli JW5020-1 Kan^{S} pBT10/pJ294[alr2355_TA_C.v.(Ct)_Ptac-synUcTE]/pCDF[affA1_Ab(co_Ec)-fadD_Ec]
E. coli JW5020-1 Kan^{S} pBT10/pJ294[Rleg_{—}1610_TA_C.v.(Ct)_Ptac-synUcTE]/pCDF[wax-dgaT_AsADP1(co_Ec)-fadD_Ec]
E. coli JW5020-1 Kan^{S} pBT10/pJ294[Rleg_{—}1610_TA_C.v.(Ct)_Ptac-synUcTE]/pCDF[affA1_Ab(co_Ec)-fadD_Ec]
E. coli JW5020-1 Kan^{S} pBT10/pJ294[blr1738_TA_C.v.(Ct)_Ptac-synUcTE]/pCDF[wax-dgaT_AsADP1(co_Ec)-fadD_Ec]
E. coli JW5020-1 Kan^{S} pBT10/pJ294[blr1738_TA_C.v.(Ct)_Ptac-synUcTE]/pCDF[atfA1_Ab(co_Ec)-fadD_Ec]
E. coli JW5020-1 Kan^{S} pBT10/pJ294[BMD_{—}5199_TA_C.v.(Ct)_Ptac-synUcTE]/pCDF[wax-dgaT_AsADP1(co_Ec)-fadD_Ec]
E. coli JW5020-1 Kan^{S} pBT10/pJ294[BMD_{—}5199_TA_C.v.(Ct)_Ptac-synUcTE]/pCDF[atfA1_Ab(co_Ec)-fadD_Ec]

These strains are subjected to a fed-batch fermentation in order to analyse their capacity for production of methyl hydroxylaurate, methyl oxolaurate, methyl carboxylaurate and methyl aminolaurate and methyl hydroxytetradecanoate, methyl oxotetradecanoate, methyl carboxytetradecanoate and methyl aminotetradecanoate from glucose. This is performed with an 8-fold parallel fermentation system from DASGIP, as described in Example 8.

It is shown that these strains, with addition of 1% (v/v) methanol, are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate from glucose.

### Example 15

### Production of Aminolauric Acid by an E. coli Strain with Deletion in the Gene fadE and Expression Vectors for the Genes synUcTE from Umbellularia californica, Alr2355 from Nostoc sp. PCC 7120, Rleg_{—}1610 from Rhizobium leguminasorum bv. trifolii WSM1325, blr1738 (aldA) from Bradyrhizobium japonicum USDA 110 or BMD 5199 from Bacillus megaterium DSM 319 and Cv_{—}2025 from Chromobacterium violaceum in Combination with an Expression Vector for the Genes alkB, alkG, alkT and alkL from the Alk Operon of Pseudomonas putida

To create E. coli strains with expression vectors for the genes synUcTE from Umbellularia californica, alr2355 from Nostoc sp. PCC 7120 (SEQ ID No. 85), Rleg_{—}1610 from Rhizobium leguminasorum bv. trifolii WSM1325 (SEQ ID No. 87), blr1738 (aldA) from Bradyrhizobium japonicum USDA 110 (SEQ ID No. 89) or BMD 5199 from Bacillus megaterium DSM 319 (SEQ ID No. 91) and Cv_{—}2025 from Chromobacterium violaceum in combination with an expression vector for the genes alkB, alkG and alkT from the alk operon of Pseudomonas putida GPo1, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[alr2355_TA_C.v.(Ct)_Ptac-synUcTE] (SEQ ID No. 93), pJ294[Rleg_{—}1610_TA_C.v.(Ct)_Ptac-synUcTE] (SEQ ID No. 94), pJ294[blr1738_TA_C.v.(Ct)_Ptac-synUcTE] (SEQ ID No. 95), or pJ294[BMD_{—}5199_TA_C.v.(Ct)_Ptac-synUcTE] (SEQ ID No. 96)
   and plated out onto LB agar plates containing kanamycin (50 μg/ml) and ampicillin (100 μg/ml). Transformants are checked for the presence of the correct plasmids by plasmid preparation and analytical restriction analysis.

In this manner, the following strains are constructed:
E. coli JW5020-1 Kan^{S} pBT10/pJ294[alr2355_TA_C.v.(Ct)_Ptac-synUcTE]
E. coli JW5020-1 Kan^{S} pBT10/pJ294[Rleg_{—}1610_TA_C.v.(Ct)_Ptac-synUcTE]
E. coli JW5020-1 Kan^{S} pBT10/pJ294[blr1738_TA_C.v.(Ct)_Ptac-synUcTE]
E. coli JW5020-1 Kan^{S} pBT10/pJ294[BMD_{—}5199_TA_C.v.(Ct)_Ptac-synUcTE]

These strains are subjected to a fed-batch fermentation in order to analyse their capacity for production of hydroxylauric acid, oxolauric acid, carboxylauric acid and aminolauric acid and hydroxytetradecanoic acid, oxotetradecanoic acid, carboxytetradecanoic acid and aminotetradecanoic acid from glucose. This is performed with an 8-fold parallel fermentation system from DASGIP, as described in Example 8.

It is shown that these strains are capable of forming lauric acid, hydroxylauric acid, oxolauric acid, carboxylauric acid and aminolauric acid and tetradecanoic acid, hydroxytetradecanoic acid, oxotetradecanoic acid, carboxytetradecanoic acid and aminotetradecanoic acid from glucose.

### Example 16

### Production of Methyl Aminolaurate by an E. coli Strain with Deletion in the Gene fadE and Expression Vectors for the Genes synUcTE from Umbellularia californica and Cv_{—}2025 from Chromobacterium violaceum in Combination with an Expression Vector for the Genes alkB, alkG, alkT and alkL from the Alk Operon of Pseudomonas putida and an Expression Vector for the Genes fadD from Escherichia coli and Wax-dgaT from Acinetobacter sp. ADP1 or atfA 1 from Alcanivorax borkumensis

To create E. coli strains with expression vectors for the genes synUcTE from Umbellularia californica and Cv_{—}2025 from Chromobacterium violaceum in combination with an expression vector for the genes alkB, alkG and alkT from the alk operon of Pseudomonas putida GPo1 and an expression vector for the genes fadD from Escherichia coli and wax-dgaT from Acinetobacter sp. ADP1 or atfA1 from Alcanivorax borkumensis, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[TA_C.v.(Ct)_Ptac-synUcTE] (SEQ ID No. 97), and
3. pCDF[wax-dgaT_AsADP1(co_Ec)-fadD_Ec] (SEQ ID No. 16), or pCDF[atfA1_Ab(co_Ec)-fadD_Ec] (SEQ ID No. 17)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmid pJ294[TA_C.v.(Ct)_Ptac-synUcTE] (SEQ ID No. 97) is created starting from the plasmid pJ294[alaDH_B.s._TA_C.v.(Ct)_Ptac-synUcTE] (SEQ ID No. 38), in that parts of the gene coding for the alanine dehydrogenase from Bacillus subtilis essential for functional expression are cut out from the vector with PmeI/SnaBI and this is then religated.

In this manner, the following strains are constructed:
E. coli JW5020-1 Kan^{S} pBT10/pJ294[TA_C.v.(Ct)_Ptac-synUcTE]/pCDF[wax-dgaT_AsADP1(co_Ec)-fadD_Ec]
E. coli JW5020-1 Kan^{S} pBT10/pJ294[TA_C.v.(Ct)_Ptac-synUcTE]/pCDF[atfA1_Ab(co_Ec)-fadD_Ec]

These strains are subjected to a fed-batch fermentation in order to analyse their capacity for production of methyl hydroxylaurate, methyl oxolaurate, methyl carboxylaurate and methyl aminolaurate and methyl hydroxytetradecanoate, methyl oxotetradecanoate, methyl carboxytetradecanoate and methyl aminotetradecanoate from glucose. This is performed with an 8-fold parallel fermentation system from DASGIP, as described in Example 8.

It is shown that these strains, with addition of 1% (v/v) methanol, are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate from glucose.

### Example 17

### Production of Aminolauric Acid by an E. coli Strain with Deletion in the Gene fadE and Expression Vectors for the Genes synUcTE from Umbellularia californica and Cv_{—}2025 from Chromobacterium violaceum in Combination with an Expression Vector for the Genes alkB, alkG, alkT and alkL from the Alk Operon of Pseudomonas putida

To create E. coli strains with expression vectors for the genes synUcTE from Umbellularia californica and Cv_{—}2025 from Chromobacterium violaceum in combination with an expression vector for the genes alkB, alkG and alkT from the alk operon of Pseudomonas putida GPo1, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[TA_C.v.(Ct)_Ptac-synUcTE] (SEQ ID No. 97)
   and plated out onto LB agar plates containing kanamycin (50 μg/ml) and ampicillin (100 μg/ml). Transformants are checked for the presence of the correct plasmids by plasmid preparation and analytical restriction analysis.

In this manner, the following strain is constructed:
E. coli JW5020-1 Kan^{S} pBT10/pJ294[TA_C.v.(Ct)_Ptac-synUcTE]

This strain is subjected to a fed-batch fermentation in order to analyse its capacity for the production of hydroxylauric acid, oxolauric acid, carboxylauric acid and aminolauric acid and hydroxytetradecanoic acid, oxotetradecanoic acid, carboxytetradecanoic acid and aminotetradecanoic acid from glucose. This is performed with an 8-fold parallel fermentation system from DASGIP, as described in Example 8.

It is shown that this strain is capable of producing lauric acid, hydroxylauric acid, oxolauric acid, carboxylauric acid and aminolauric acid and tetradecanoic acid, hydroxytetradecanoic acid, oxotetradecanoic acid, carboxytetradecanoic acid and aminotetradecanoic acid from glucose.

### Example 18

### Production of methyl aminolaurate by an E. coli strain with deletion in the gene fadE and an expression vector for the gene synUcTE from Umbellularia californica in combination with an expression vector for the genes alkB, alkG, alkT and alkL from the alk operon of Pseudomonas putida and an expression vector for the gene fadD from Escherichia coli and wax-dgaT from Acinetobacter sp. ADP1 or atfA 1 from Alcanivorax borkumensis

To create E. coli strains with an expression vector for the gene synUcTE from Umbellularia californica in combination with an expression vector for the genes alkB, alkG and alkT from the alk operon of Pseudomonas putida GPo1 and an expression vector for the genes fadD from Escherichia coli and wax-dgaT from Acinetobacter sp. ADP1 or atfA1 from Alcanivorax borkumensis, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[Ptac-synUcTE] (SEQ ID No. 98), and
3. pCDF[wax-dgaT_AsADP1(co_Ec)-fadD_Ec] (SEQ ID No. 16), or pCDF[affA1_Ab(co_Ec)-fadD_Ec] (SEQ ID No. 17)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmid pJ294[Ptac-synUcTE] (SEQ ID No. 98) is created starting from the plasmid pJ294[alaDH_B.s._TA_C.v.(Ct)_Ptac-synUcTE] (SEQ ID No. 38), in that parts of the genes coding for the alanine dehydrogenase from Bacillus subtilis and the transaminase Cv_{—}2025 from Chromobacterium violaceum essential for functional expression are cut out from the vector with SrfI/SnaBI and this is then religated.

In this manner, the following strains are constructed:
E. coli JW5020-1 Kan^{S} pBT10/pJ294[Ptac-synUcTE]/pCDF[wax-dgaT_AsADP1(co_Ec)-fadD_Ec]
E. coli JW5020-1 Kan^{S} pBT10/pJ294[Ptac-synUcTE]/pCDF[atfA1_Ab(co_Ec)-fadD_Ec]

These strains are subjected to a fed-batch fermentation in order to analyse their capacity for production of methyl hydroxylaurate, methyl oxolaurate and methyl carboxylaurate and methyl hydroxytetradecanoate, methyl oxotetradecanoate and methyl carboxytetradecanoate from glucose. This is performed with an 8-fold parallel fermentation system from DASGIP, as described in Example 8.

It is shown that these strains, with addition of 1% (v/v) methanol, are capable of forming methyl laurate, methyl hydroxylaurate, methyl oxolaurate and methyl carboxylaurate and methyl tetradecanoate, methyl hydroxytetradecanoate, methyl oxotetradecanoate and methyl carboxytetradecanoate from glucose.

### Example 19

### Production of Aminolauric Acid by an E. coli Strain with Deletion in the Gene fadE and an Expression Vector for the Gene synUcTE from Umbellularia californica in Combination with an Expression Vector for the Genes alkB, alkG, alkT and alkL from the Alk Operon of Pseudomonas putida

To create E. coli strains with an expression vector for the gene synUcTE from Umbellularia californica in combination with an expression vector for the genes alkB, alkG and alkT from the alk operon of Pseudomonas putida GPo1, electrocompetent cells of E. coli JW5020-1 Kan^{S} are produced. This takes place in a manner known to those skilled in the art.

JW5020-1 Kan^{S} is sequentially transformed with the plasmids
1. pBT10 (construction described in practical example B.2 of PCT/EP2008/067447), and
2. pJ294[Ptac-synUcTE] (SEQ ID No. 96)
   and plated out onto LB agar plates containing kanamycin (50 μg/ml) and ampicillin (100 μg/ml). Transformants are checked for the presence of the correct plasmids by plasmid preparation and analytical restriction analysis.

In this manner, the following strain is constructed:
E. coli JW5020-1 Kan^{S} pBT10/pJ294[Ptac-synUcTE]

This strain is subjected to a fed-batch fermentation in order to analyse its capacity for the production of hydroxylauric acid, oxolauric acid and carboxylauric acid and hydroxytetradecanoic acid, oxotetradecanoic acid and carboxytetradecanoic acid from glucose. This is performed with an 8-fold parallel fermentation system from DASGIP, as described in Example 8. It is shown that this strain is capable of producing lauric acid, hydroxylauric acid, oxolauric acid and carboxylauric acid and tetradecanoic acid, hydroxytetradecanoic acid, oxotetradecanoic acid and carboxytetradecanoic acid from glucose.

### Example 20

### Production of Methyl Aminohexanoate, Methyl Aminooctanoate, Methyl Aminodecanoate, Methyl Aminotetradecanoate and Methyl Amino-9-Hexadecenoate by E. coli Strains with Deletion of the fadE Gene and Expression Vectors for the Genes synUcTE from Umbellularia californica, ChFATB2 from Cuphea hookeriana, CnFATB3 from Cocos nucifera or CPF 2954 from Clostridium perfringens and Ald from Bacillus subtilis, Cv_{—}2025 from Chromobacterium violaceum in Combination with an Expression Vector for the Genes alkB, alkG and alkT from the Alk Operon of Pseudomonas putida and an Expression Vector for the Gene Mmar_{—}3356 from Mycobacterium marinum or the Gene ′tesA* from E. coli

To create E. coli strains with expression vectors for the genes synUcTE from Umbellularia californica, ChFATB2 from Cuphea hookeriana (SEQ ID No. 58), CnFATB3 from Cocos nucifera (SEQ ID No. 60) or CPF 2954 from Clostridium perfringens (SEQ ID No. 62) and ald from Bacillus subtilis, and Cv_{—}2025 from Chromobacterium violaceum in combination with an expression vector for the genes alkB, alkG and alkT from the alk operon of Pseudomonas putida GPo1 and an expression vector for the gene Mmar_{—}3356 from Mycobacterium marinum (SEQ ID No. 99) or the gene ′tesA* from E. coli (SEQ ID No. 101), electrocompetent cells of E. coli JW5020-1 Kan^{S} are produced. This takes place in a manner known to those skilled in the art.

JW5020-1 Kan^{S} is sequentially transformed with the plasmids
pBT10 (construction described in practical example B.2 of PCT/EP2008/067447), and
pJ294[alaDH_B.s._TA_C.v.(Ct)_Ptac-synUcTE] (SEQ ID No. 38), pJ294[alaDH_B.s._TA_C.v.(Ct)_Ptac-ChFATB2] (SEQ ID No. 70), pJ294[alaDH_B.s._TA_C.v.(Ct)_Ptac-CnFATB3] (SEQ ID No. 71), or pJ294[alaDH_B.s._TA_C.v.(Ct)_Ptac-CPF_{—}2954] (SEQ ID No. 72), and
pCDF[Mmar_{—}3356] (SEQ ID No. 103), or pCDF[Ec′tesA*] (SEQ ID No. 104)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmids pCDF[Mmar_{—}3356] (SEQ ID No. 103) and pCDF[Ec′tesA*] (SEQ ID No. 104) are created starting from the plasmid pCDF[wax-dgaT_AsADP1(co_Ec)-fadD_Ec] (SEQ ID No. 16), in that the wax-dgaT gene from Acinetobacter sp. ADP1 and the fadD gene from E. coli are cut out from the vector with BamHI/XhoI incl. P_{tac} and the 3′-flanking region of fadD and replaced by the genes coding for the fatty acid O-methyltransferase Mmar_{—}3356 (SEQ ID No. 100) or the acyl-ACP: methanol O-methyltransferase ′TesA* (SEQ ID No. 102) (incl. P_{tac} and the region identical to the 3′-flanking region of fadD). These fragments are created by gene synthesis, wherein the region coding for the fatty acid O-methyltransferase is codon-optimized for translation in E. coli, while the region coding for the thioester transesterase ′TesA* is not codon-optimized for translation in E. coli, but instead the wild type sequence is used.

In this manner, the following strains are constructed:
E. coli JW5020-1 Kan^{S} pBT10/pJ294[alaDH_B.s._TA_C.v.(Ct)_Ptac-synUcTE]/pCDF[Mmar_{—}3356]
E. coli JW5020-1 Kan^{S} pBT10/pJ294[alaDH_B.s._TA_C.v.(Ct)_Ptac-ChFATB2]/pCDF[Mmar_{—}3356]
E. coli JW5020-1 Kan^{S} pBT10/pJ294[alaDH_B.s._TA_C.v.(Ct)_Ptac-CnFATB3]/pCDF[Mmar_{—}3356]
E. coli JW5020-1 Kan^{S} pBT10/pJ294[alaDH_B.s._TA_C.v.(Ct)_Ptac-CPF_{—}2954]/pCDF[Mmar_{—}3356]
E. coli JW5020-1 Kan^{S} pBT10/pJ294[alaDH_B.s._TA_C.v.(Ct)_Ptac-synUcTE]/pCDF[Ec′tesA*]
E. coli JW5020-1 Kan^{S} pBT10/pJ294[alaDH_B.s._TA_C.v.(Ct)_Ptac-ChFATB2]/pCDF[Ec′tesA*]
E. coli JW5020-1 Kan^{S} pBT10/pJ294[alaDH_B.s._TA_C.v.(Ct)_Ptac-CnFATB3]/pCDF[Ec′tesA*]
E. coli JW5020-1 Kan^{S} pBT10/pJ294[alaDH_B.s._TA_C.v.(Ct)_Ptac-CPF_{—}2954]/pCDF[Ec′tesA*]

These strains are subjected to a fed-batch fermentation in order to analyse their capacity for production of hydroxy fatty acid methyl esters, oxo fatty acid methyl esters, carboxy fatty acid methyl esters and amino fatty acid methyl esters from glucose. This is performed with an 8-fold parallel fermentation system from DASGIP, as described in Example 8.

It is shown that the strains E. coli JW5020-1 Kan^{S} pBT10/pJ294[alaDH_B.s._TA_C.v.(Ct)_Ptac-synUcTE]/pCDF[Mmar_{—}3356] and E. coli JW5020-1 Kan^{S} pBT10/pJ294[alaDH_B.s._TA_C.v.(Ct)_Ptac-synUcTE]/pCDF[Ec′tesA*], with addition of 1% (v/v) methanol, are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate from glucose.

It is shown that the strains E. coli JW5020-1 Kan^{S} pBT10/pJ294[alaDH_B.s._TA_C.v.(Ct)_Ptac-ChFATB2]/pCDF[Mmar_{—}3356] and E. coli JW5020-1 Kan^{S} pBT10/pJ294[alaDH_B.s._TA_C.v.(Ct)_Ptac-ChFATB2]/pCDF[Ec′tesA*], with addition of 1% (v/v) methanol, are capable of forming methyl octanoate, methyl hydroxyoctanoate, methyl oxooctanoate, methyl carboxyoctanoate and methyl aminooctanoate and methyl decanoate, methyl hydroxydecanoate, methyl oxodecanoate, methyl carboxydecanoate and methyl aminodecanoate from glucose.

It is shown that the strains E. coli JW5020-1 Kan^{S} pBT10/pJ294[alaDH_B.s._TA_C.v.(Ct)_Ptac-CnFATB3]/pCDF[Mmar_{—}3356] and E. coli JW5020-1 Kan^{S} pBT10/pJ294[alaDH_B.s._TA_C.v.(Ct)_Ptac-CnFATB3]/pCDF[Ec′tesA*], with addition of 1% (v/v) methanol, are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate and methyl 9-hexadecenoate, methyl hydroxy-9-hexadecenoate, methyl oxo-9-hexadecenoate, methyl carboxy-9-hexadecenoate and methyl amino-9-hexadecenoate from glucose.

It is shown that the strains E. coli JW5020-1 Kan^{S} pBT10/pJ294[alaDH_B.s._TA_C.v.(Ct)_Ptac-CnFATB3]/pCDF[Mmar_{—}3356] and E. coli JW5020-1 Kan^{S} pBT10/pJ294[alaDH_B.s._TA_C.v.(Ct)_Ptac-CnFATB3]/pCDF[Ec′tesA*], with addition of 1% (v/v) methanol, are capable of forming methyl octanoate, methyl hydroxyoctanoate, methyl oxooctanoate, methyl carboxyoctanoate and methyl aminooctanoate and methyl hexanoate, methyl hydroxyhexanoate, methyl oxohexanoate, methyl carboxyhexanoate and methyl aminohexanoate from glucose.

### Example 21

### Production of Methyl Aminolaurate and Methyl Aminotetradecanoate by E. coli Strains with Deletion of the Gene fadE and Expression Vectors for the Genes synUcTE from Umbellularia californica, and Ald from Bacillus subtilis, and Cv_{—}2025 from Chromobacterium violaceum in Combination with an Expression Vector for the Genes alkM, alkG and alkT from the Alk Operon of Acinetobacter sp. ADP1 or alkS, alkT, alkB1, alkG and alkT from the Alk Operon of Marinobacter aquaeoli VT8 and an Expression Vector for the Genes fadD from Escherichia coli and Wax-dgaT from Acinetobacter sp. ADP1 or affA1 from Alcanivorax borkumensis

To create E. coli strains with expression vectors for the genes synUcTE from Umbefiularia californica and ald from Bacillus subtilis and Cv_{—}2025 from Chromobacterium violaceum in combination with expression vectors for the genes alkM, alkG and alkT from the alk operon of Acinetobacter sp. ADP1 (SEQ ID No. 105) or alkS, alkT, alkB1, alkG and alkT from the alk operon of Marinobacter aquaeoli VT8 (SEQ ID No. 106) and an expression vector for the genes fadD from Escherichia coli and wax-dgaT from Acinetobacter sp. ADP1 or atfA1 from Alcanivorax borkumensis SK2, electrocompetent cells of E. coli JW5020-1 Kan^{S} are produced. This takes place in a manner known to those skilled in the art.

JW5020-1 Kan^{S} is sequentially transformed with the plasmids
4. pJ294[alaDH_B.s._TA_C.v.(Ct)_Ptac-synUcTE] (SEQ ID No. 38),
5. pCDF[wax-dgaT_AsADP1(co_Ec)-fadD_Ec] (SEQ ID No. 16) bzw. pCDF[atfA1_Ab(co_Ec)-fadD_Ec] (SEQ ID No. 17)
6. pCOM10-AcalkMGT (SEQ ID No. 107) pCOM10-MaalkST-B1G (SEQ ID No. 108)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmids pCOM10-AcalkMGT (SEQ ID No. 107) and pCOM10-MaalkST-B1G (SEQ ID No. 108) are created starting from the plasmid pCOM10 (SEQ ID No. 109; Smits T H, Seeger M A, Witholt B, van Beilen J B. New alkane-responsive expression vectors for Escherichia coli and Pseudomonas. Plasmid. 2001. 46(1):16-24.). For this, pCOM10 is cleaved with XhoI/BamHI (pCOM10-MaalkST-B1G) or EcoRI (pCOM10-AcalkMGT) and the loci Acinetobacter sp. ADP1 alkMGT or Marinobacter aquaeoli VT8 alkST-alkB1G respectively inserted into pCOM10. These loci are created by gene synthesis, wherein the regions coding for Acinetobacter sp. ADP1 AlkM (SEQ ID No. 110), AlkG (SEQ ID No. 111) and AlkT (SEQ ID No. 112) and those coding for Marinobacter aquaeoli VT8 AlkS (SEQ ID No. 113), AlkT (SEQ ID No. 114), AlkB1 (SEQ ID No. 115) and AlkG (SEQ ID No. 116) are not codon-optimized for translation in E. coli, but instead the wild type sequence is used.

In this manner, the following strains are constructed:
E. coli JW5020-1 Kan^{S} pCOM10-AcalkMGT/pJ294[alaDH_B.s._TA_C.v.(Ct)_Ptac-synUcTE]/pCDF[wax-dgaT_AsADP1(co_Ec)-fadD_Ec]
E. coli JW5020-1 Kan^{S} pCOM10-AcalkMGT/pJ294[alaDH_B.s._TA_C.v.(Ct)_Ptac-synUcTE]/pCDF[atfA1_Ab(co_Ec)-fadD_Ec]
E. coli JW5020-1 Kan^{S} pCOM10-MaalkST-B1G/pJ294[alaDH_B.s._TA_C.v.(Ct)_Ptac-synUcTE]/pCDF[wax-dgaT_AsADP1(co_Ec)-fadD_Ec]
E. coli JW5020-1 Kan^{S} pCOM10-MaalkST-B1G/pJ294[alaDH_B.s._TA_C.v.(Ct)_Ptac-synUcTE]/pCDF[atfA1_Ab(co_Ec)-fadD_Ec]

These strains are subjected to a fed-batch fermentation in order to analyse their capacity for production of hydroxy fatty acid methyl esters, oxo fatty acid methyl esters, carboxy fatty acid methyl esters and amino fatty acid methyl esters from glucose. This is performed with an 8-fold parallel fermentation system from DASGIP, as described in Example 8.

It is shown that, with addition of 1% (v/v) methanol, these strains are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate from glucose.

### Example 22

### Production of Aminolauric Acid and Aminotetradecanoic Acid by E. coli Strains with Deletion of the Gene fadE and Expression Vectors for the Genes synUcTE from Umbellularia californica, and ald from Bacillus subtilis, and Cv_{—}2025 from Chromobacterium violaceum in Combination with an Expression Vector for the Genes alkM, alkG and alkT from the Alk Operon of Acinetobacter sp. ADP1 or alkS, alkT, alkB1, alkG and alkT from the Alk Operon of Marinobacter aquaeoli VT8

To create E. coli strains with expression vectors for the genes synUcTE from Umbellularia californica and ald from Bacillus subtilis and Cv_{—}2025 from Chromobacterium violaceum in combination with expression vectors for the genes alkM, alkG and alkT from the alk operon of Acinetobacter sp. ADP1 (SEQ ID No. 105) or alkS, alkT, alkB1, alkG and alkT from the alk operon of Marinobacter aquaeoli VT8 (SEQ ID No. 106), electrocompetent cells of E. coli JW5020-1 Kan^{S} are produced. This takes place in a manner known to those skilled in the art.

JW5020-1 Kan^{S} is sequentially transformed with the plasmids
1. pJ294[alaDH_B.s._TA_C.v.(Ct)_Ptac-synUcTE] (SEQ ID No. 38),
2. pCOM10-AcalkMGT (SEQ ID No. 107) pCOM10-MaalkST-B1G (SEQ ID No. 108)
   and plated out onto LB agar plates containing kanamycin (50 μg/ml) and ampicillin (100 μg/ml). Transformants are checked for the presence of the correct plasmids by plasmid preparation and analytical restriction analysis.

In this manner, the following strains are constructed:
E. coli JW5020-1 Kan^{S} pCOM10-AcalkMGT/pJ294[alaDH_B.s._TA_C.v.(Ct)_Ptac-synUcTE]
E. coli JW5020-1 Kan^{S} pCOM10-AcalkMGT/pJ294[alaDH_B.s._TA_C.v.(Ct)_Ptac-synUcTE]
E. coli JW5020-1 Kan^{S} pCOM10-MaalkST-B1G/pJ294[alaDH_B.s._TA_C.v.(Ct)_Ptac-synUcTE]
E. coli JW5020-1 Kan^{S} pCOM10-MaalkST-B1G/pJ294[alaDH_B.s._TA_C.v.(Ct)_Ptac-synUcTE]

These strains are subjected to a fed-batch fermentation in order to analyse their capacity for production of hydroxy fatty acids, oxo fatty acids, carboxy fatty acids and amino fatty acids from glucose. This is performed with an 8-fold parallel fermentation system from DASGIP, as described in Example 8.

It is shown that these strains are capable of forming lauric acid, hydroxylauric acid, oxolauric acid, carboxylauric acid and aminolauric acid and tetradecanoic acid, hydroxytetradecanoic acid, oxotetradecanoic acid, carboxytetradecanoic acid and aminotetradecanoic acid from glucose.

### Example 23

### Production of Methyl Aminolaurate and Methyl Aminotetradecanoate by E. coli Strains with Deletion of the Gene fadE and Expression Vectors for the Genes synUcTE from Umbellularia californica, and Ald from Bacillus subtilis, and Cv_{—}2025 from Chromobacterium violaceum in Combination with an Expression Vector for the Genes ABO_{—}0200, ABO_{—}0201 and ABO_{—}0203 from Alcanivorax borkumensis SK2 and an Expression Vector for the Genes fadD from Escherichia coli and Wax-dgaT from Acinetobacter sp. ADP1 or atfA1 from Alcanivorax borkumensis

To create E. coli strains with expression vectors for the genes synUcTE from Umbellularia californica and ald from Bacillus subtilis and Cv_{—}2025 from Chromobacterium violaceum in combination with an expression vector for the genes ABO_{—}0200, ABO_{—}0201 and ABO_{—}0203 from Alcanivorax borkumensis SK2 (SEQ ID No. 117), coding for a ferredoxin (ABO_{—}0200; SEQ ID No. 118), a CYP_{—}153 monooxygenase (ABO_{—}0201; SEQ ID No. 119) and a ferredoxin oxidoreductase (ABO_{—}0203; SEQ ID No. 120) and an expression vector for the genes fadD from Escherichia coli and wax-dgaT from Acinetobacter sp. ADP1 or atfA1 from Alcanivorax borkumensis SK2, electrocompetent cells of E. coli JW5020-1 Kan^{S} are produced. This takes place in a manner known to those skilled in the art.

JW5020-1 Kan^{S} is sequentially transformed with the plasmids
1. pJ294[alaDH_B.s._TA_C.v.(Ct)_Ptac-synUcTE] (SEQ ID No. 38),
2. pCDF[wax-dgaT_AsADP1(co_Ec)-fadD_Ec] (SEQ ID No. 16) bzw. pCDF[atfA1_Ab(co_Ec)-fadD_Ec] (SEQ ID No. 17)
3. pCOM10-AbCYP_{—}153 (SEQ ID No. 121)
   and plated out onto LB agar plates containing kanamycin (50 μg/ml), ampicillin (100 μg/ml) and spectinomycin (100 μg/ml). Transformants are checked for the presence of the correct plasmids by plasmid preparation and analytical restriction analysis.

The plasmid pCOM10-AbCYP_{—}153 (SEQ ID No. 121) is created starting from the plasmid pCOM10 (SEQ ID No. 97; Smits T H, Seeger M A, Witholt B, van Beilen J B. New alkane-responsive expression vectors for Escherichia coli and Pseudomonas. Plasmid. 2001. 46(1):16-24.). For this, pCOM10 is cleaved with EcoRI/SalI and the fragment containing the genes ABO_{—}0200, ABO_{—}0201 and ABO_{—}0203 from Alcanivorax borkumensis SK2 inserted.

This fragment is created by gene synthesis, wherein the sections from Alcanivorax borkumensis SK2 coding for the ferredoxin (ABO_{—}0200; SEQ ID No. 106), the CYP_{—}153 monooxygenase ABO_{—}0201; SEQ ID No. 107) and the ferredoxin oxidoreductase (ABO_{—}0203; SEQ ID No. 108) are not codon-optimized for translation in E. coli, but instead the wild type sequence is used.

In this manner, the following strains are constructed:
E. coli JW5020-1 Kan^{S} pCOM10-AbCYP_{—}153/pJ294[alaDH_B.s._TA_C.v.(Ct)_Ptac-synUcTE]/pCDF[wax-dgaT_AsADP1(co_Ec)-fadD_Ec]
E. coli JW5020-1 Kan^{S} pCOM10-AbCYP_{—}153/pJ294[alaDH_B.s._TA_C.v.(Ct)_Ptac-synUcTE]/pCDF[atfA1_Ab(co_Ec)-fadD_Ec]

These strains are subjected to a fed-batch fermentation in order to analyse their capacity for production of hydroxy fatty acid methyl esters, oxo fatty acid methyl esters, carboxy fatty acid methyl esters and amino fatty acid methyl esters from glucose. This is performed with an 8-fold parallel fermentation system from DASGIP, as described in Example 8.

It is shown that, with addition of 1% (v/v) methanol, these strains are capable of forming methyl laurate, methyl hydroxylaurate, methyl oxolaurate, methyl carboxylaurate and methyl aminolaurate and methyl tetradecanoate, methyl hydroxytetradecanoate, methyl oxotetradecanoate, methyl carboxytetradecanoate and methyl aminotetradecanoate from glucose.

### Example 24

### Production of Aminolauric Acid and Aminotetradecanoic Acid by E. coli Strains with Deletion of the Gene fadE and Expression Vectors for the Genes synUcTE from Umbellularia californica, and ald from Bacillus subtilis, and Cv_{—}2025 from Chromobacterium violaceum in Combination with an Expression Vector for the Genes ABO_{—}0200, ABO_{—}0201 and ABO_{—}0203 from Alcanivorax borkumensis SK2

To create E. coli strains with expression vectors for the genes synUcTE from Umbellularia californica and ald from Bacillus subtilis and Cv_{—}2025 from Chromobacterium violaceum in combination with an expression vector for the genes ABO_{—}0200, ABO_{—}0201 and ABO_{—}0203 from Alcanivorax borkumensis SK2 (SEQ ID No. 117), coding for a ferredoxin (ABO_{—}0200; SEQ ID No. 118), a CYP_{—}153 monooxygenase (ABO_{—}0201; SEQ ID No. 119) and a ferredoxin oxidoreductase (ABO_{—}0203; SEQ ID No. 120), electrocompetent cells of E. coli JW5020-1 Kan^{S} are produced. This takes place in a manner known to those skilled in the art.

JW5020-1 Kan^{S} is sequentially transformed with the plasmids
1. pJ294[alaDH_B.s._TA_C.v.(Ct)_Ptac-synUcTE] (SEQ ID No. 38) and
2. pCOM10-AbCYP_{—}153 (SEQ ID No. 121) and plated out onto LB agar plates containing kanamycin (50 μg/ml) and ampicillin (100 μg/ml). Transformants are checked for the presence of the correct plasmids by plasmid preparation and analytical restriction analysis.

In this manner, the following strain is constructed:
E. coli JW5020-1 Kan^{S} pCOM10-AbCYP_{—}153/pJ294[alaDH_B.s._TA_C.v.(Ct)_Ptac-synUcTE]

This strain is subjected to a fed-batch fermentation in order to analyse its capacity for the production of hydroxy fatty acids, oxo fatty acids, carboxy fatty acids and amino fatty acids from glucose. This is performed with an 8-fold parallel fermentation system from DASGIP, as described in Example 8.

It is shown that this strain is capable of forming lauric acid, hydroxylauric acid, oxolauric acid, carboxylauric acid and aminolauric acid and tetradecanoic acid, hydroxytetradecanoic acid, oxotetradecanoic acid, carboxytetradecanoic acid and aminotetradecanoic acid from glucose.

1. A microorganism comprising:
a first genetic engineering modification, so that compared to its wild type it is capable of forming more carboxylic acid or carboxylate ester from at least one simple carbon source, and
a second genetic engineering modification, such that the microorganism in comparison to its wild type displays increased activity of at least one enzyme E₁ that catalyzes the conversion of a carboxylic acid or a carboxylate ester to a corresponding ω-hydroxycarboxylic acid or ω-hydroxy-carboxylate ester.

2. The microorganism of claim 1, wherein the enzyme E₁ is an E₁ₐ P450 alkane hydroxylase or an E_{1b} AlkB alkane hydroxylase of EC 1.14.15.3.

3. The microorganism of claim 1, wherein the second genetic modification is such that the microorganism in comparison to its wild type displays increased activity of at least one enzyme selected from the group consisting of:
an enzyme E₂ that catalyzes the conversion of an ω-oxocarboxylic acid or an ω-oxocarboxylate ester to the corresponding ω-aminocarboxylic acid or ω-aminocarboxylate ester,
an enzyme E₃ that catalyzes the conversion of an α-ketocarboxylic acid to an amino acid,
an enzyme E₄ that catalyzes the conversion of an ω-hydroxycarboxylic or an ω-hydroxycarboxylate ester to the corresponding ω-oxocarboxylic acid or ω-oxocarboxylate ester, and
an enzyme E₅ that catalyzes the conversion of an ω-oxocarboxylic acid or an ω-oxocarboxylate ester to the corresponding ω-carboxycarboxylic acid or ω-carboxycarboxylate ester.

4. The microorganism of claim 3, wherein the second genetic modification comprises a combination of increased activities of the enzymes selected from the group consisting of E₁E₂E₃, E₁E₂E₃E₄, E₁E₅ and E₁E₄E₅.

5. The microorganism of claim 3, wherein:
the enzyme E₂ is an ω-transaminase,
the enzyme E₃ is an alanine dehydrogenase of EC 1.4.1.1,
the enzyme E₄ is a fatty alcohol oxidase of EC 1.1.3.20, an AlkJ alcohol dehydrogenase of EC 1.1.99.- or an alcohol dehydrogenase of EC 1.1.1.1 or EC 1.1.1.2, and
the enzyme E₅ is an aldehyde dehydrogenase of EC 1.2.1.3, EC 1.2.1.4 or EC 1.2.1.5.

6. The microorganism of claim 1, wherein the enzyme E₁ is an alkane monooxygenase from Pseudomonas putida GPo1 encoded by alkBGT or a protein having a polypeptide sequence wherein up to 60% of the amino acid residues compared to the alkane monooxygenase sequence are modified by deletion, insertion, substitution or a combination thereof and which still possesses at least 50% of the activity of the alkane monooxygenase.

7. The microorganism of claim 1, wherein the second genetic modification is such that compared to its wild type the microorganism forms more alkL gene product.

8. The microorganism of claim 7, wherein the alkL gene product is encoded by an alkL gene of gram-negative bacteria.

9. The microorganism of claim 7, wherein the alkL gene product is selected from the group consisting of:
a protein encoded by SEQ ID NO:1,
a protein encoded by SEQ ID NO:3,
a protein comprising a polypeptide sequence selected from the group consisting of SEQ ID NOs: 2, 4, 5, 6 and 7, and
a protein having a polypeptide sequence wherein up to 60% of the amino acid residues compared to SEQ ID NO: 2, 4, 5, 6 or 7 are modified by deletion, insertion, substitution or a combination thereof and which still possesses at least 50% of the activity of the protein with the respective reference sequence SEQ ID NO: 2, 4, 5, 6 or 7.

10. The microorganism of claim 1, which is a species selected from the group consisting of:
E. coli, Pseudomonas sp., Pseudomonas fluorescens, Pseudomonas putida, Pseudomonas stutzeri, Acinetobacter sp., Burkholderia sp., Burkholderia thailandensis, Cyanobakterien, Klebsiella sp., Klebsiella oxytoca, Salmonella sp., Rhizobium sp. and Rhizobium meliloti, Bacillus sp., Bacillus subtilis, Clostridium sp., Corynebacterium sp., Corynebacterium glutamicum, Brevibacterium sp., Chlorella sp. and Nostoc sp.

11. The microorganism of claim 1, wherein the first genetic modification is, compared to the enzymatic activity of the wild type of the microorganism, increased activity of at least one of the enzymes selected from the group consisting of:
E₁ Acyl-ACP thioesterase,
Eᵢᵢ Acyl-CoA thioesterase,
E_{iib} Acyl-CoA:ACP transacylase,
Eᵢᵢᵢ Polyketide synthase, which catalyses a reaction which is involved in the synthesis of a carboxylic acid or a carboxylate ester, and
Eᵢᵥ Hexanoic acid synthase.

12. The microorganism of claim 1, further comprising a third genetic modification which comprises, compared with the enzymatic activity of the wild type of the microorganism, increased activity of at least one of the enzymes E_{iib}, Eᵥ, Eᵥᵢ, or Eᵥᵢᵢ which is involved in the conversion of carboxylic acids or ω-functionalized carboxylic acids to carboxylate esters or ω-functionalized carboxylate esters, selected from the group consisting of:
E_{iib} Acyl-CoA:ACP transacylase, which converts an ACP thioester into a CoA thioester or a CoA thioester into an ACP thioester,
Eᵥ Wax ester synthase or alcohol O-acyltransferase, which catalyses the synthesis of an ester from an acyl-coenzyme A thioester or an acyl-ACP thioester and an alcohol,
Eᵥᵢ Acyl-CoA (coenzyme A) synthetase, which catalyses the synthesis of an acyl-coenzyme A thioester, and
Eᵥᵢᵢ Acyl thioesterase, which catalyses the conversion of an acyl thioester with an alcohol to a carboxylate ester.

13. The microorganism of claim 12, wherein the third genetic modification comprises a combination of increased activities of the enzymes selected from the group consisting of Eᵥ, Eᵥᵢᵢ, EᵥEᵥᵢ, EᵥᵢEᵥᵢᵢ and Eᵥᵢ, EᵥᵢᵢE_{iib}.

14. The microorganism of claim 1, further comprising a fourth genetic modification which comprises, compared with the enzymatic activity of the wild type of the microorganism, increased activity of at least one of the enzymes selected from the group consisting of:
E_{iib} Acyl-CoA (coenzyme A):ACP (acyl carrier protein) transacylase, which converts an ACP thioester into a CoA thioester or a CoA thioester into an ACP thioester,
Eᵥᵢ Acyl-CoA (coenzyme A) synthetase, which preferably catalyses the synthesis of an acyl-coenzyme A thioester,
Eᵥᵢᵢᵢ Acyl-CoA (coenzyme A) reductase, which preferentially catalyses the reduction of an acyl-coenzyme A thioester to the corresponding alkan-1-al or alkan-1-ol
Eᵢₓ Fatty acid reductase, also fatty aldehyde dehydrogenase or arylaldehyde oxidoreductase, which preferentially catalyses the reduction of an alkanoic acid to the corresponding alkan-1-al, and
Eₓ Acyl-ACP (acyl carrier protein) reductase, which preferentially catalyses the reduction of an acyl-ACP thioester to the corresponding alkan-1-al or alkan-1-ol.

15. The microorganism of claim 14, wherein the second genetic modification comprises a combination of increased activities of the enzymes selected from the group consisting of Eᵥᵢᵢᵢ, Eᵢₓ, Eₓ, EᵥᵢEᵥᵢᵢᵢ, and EᵥᵢEₓE_{iib}.

16. The microorganism of claim 1, further comprising a fifth genetic modification which comprises, compared with the enzymatic activity of the wild type of the microorganism, decreased activity of at least one of the enzymes selected from the group consisting of:
Eₐ Acyl-CoA synthetase, which catalyses the synthesis of an acyl-coenzyme A thioester,
E_{b} Acyl-CoA dehydrogenase, which catalyses the oxidation of an acyl-coenzyme A thioester to the corresponding enoyl-coenzyme A thioester,
E_{c} Acyl-CoA-oxidase, which catalyses the oxidation of an acyl-coenzyme A thioester to the corresponding enoyl-coenzyme A thioester,
E_{d} Enoyl-CoA hydratase, which catalyses the hydration of an enoyl-coenzyme A thioester to the corresponding 3-hydroxyacyl-coenzyme A thioester,
Eₑ 3-hydroxyacyl-CoA dehydrogenase, which catalyses the oxidation of a 3-hydroxyacyl-coenzyme A thioester to the corresponding 3-oxoacyl-coenzyme A thioester and
E_{f} Acetyl-CoA acyltransferase, which catalyses the transfer of an acyl residue from a 3-oxoacyl-coenzyme A thioester to Coenzyme A and thereby creates an acyl-coenzyme A thioester shortened by two carbon atoms.

17. (canceled)

18. A process for producing an ω-functionalized carboxylic acid or an ω-functionalized carboxylate ester from a simple carbon source, the process comprising:
I) contacting the microorganism of claim 1 with a medium comprising a simple carbon source,
II) culturing the microorganism under conditions that enable the microorganism to form the ω-functionalized carboxylic acid or ω-functionalized carboxylate ester from the simple carbon source and
III) optionally isolating the ω-functionalized carboxylic acid or ω-functionalized carboxylate ester formed.

19. The process of claim 18, wherein the ω-functionalized carboxylic acid is an ω-aminocarboxylic acid, or wherein the ω-functionalized carboxylate ester is an ω-aminocarboxylate ester.

20. A process for producing a polyamide based on an ω-aminocarboxylic acid, the process comprising:
(a1) producing an ω-aminocarboxylic acid by the process of claim, and
(a2) polymerizing the ω-aminocarboxylic acid produced in (a1), optionally with an ω-aminocarboxylic not produced in (a1), to obtain a polyamide.

21. A polyamide obtained by the process of claim 20.

22. The process of claim 18, wherein the ω-functionalized carboxylic acid is an ω-hydroxycarboxylic acid, or wherein the ω-functionalized carboxylate ester is an ω-hydroxycarboxylate ester.

23. The process of claim 18, wherein the ω-functionalized carboxylic acid is an ω-carboxycarboxylic acid, or wherein the ω-functionalized carboxylate ester is an ω-carboxycarboxylate ester.
